(19) United States
(12) Patent Application Publication
(10) Pub. No.: US 2026/0275352 A1
(43) Pub. Date: Sep. 17, 2026

(54) COMPOSITIONS AND METHODS FOR TREATING AMYOTROPHIC LATERAL SCLEROSIS

(71) Applicant: VOYAGER THERAPEUTICS, INC., Lexington, MA (US)

(72) Inventors: Mathieu Emmanuel Nonnenmacher, Boston, MA (US); Tyler Christopher Moyer, Boston, MA (US); Jiangyu Li, Watertown, MA (US); Dan Richard Laks, Cambridge, MA (US); Cansu Colpan, North Billerica, MA (US); Michael D. Grannan, Medford, MA (US)

(21) Appl. No.: 19/472,268

(22) PCT Filed: Apr. 25, 2024
(86) PCT No.: PCT/US2024/026219

§ 371 Date: Oct. 3, 2025

Related U.S. Application Data
(60) Provisional application No. 63/462,127, filed on Apr. 26, 2023.
(60) Provisional application No. 63/545,873, filed on Oct. 26, 2023.
(60) Provisional application No. 63/564,293, filed on Mar. 12, 2024.

(51) Int. Cl. C12N 15/113; A61K 31/7105; A61P 25/28; C07K 14/005; C12N 15/86

(52) U.S. Cl. CPC C12N 15/1137; A61K 31/7105; A61P 25/28; C07K 14/005; C12N 15/86; C12N 2310/11; C12N 2310/14; C12N 2750/14122; C12N 2750/14143; C12N 2750/14145; C12N 2830/15; C12N 2830/42; C12N 2830/50; C12Y 115/01001

(57) ABSTRACT

The present disclosure relates to AAVs encoding a SOD1 targeting polynucleotide which may be used to treat amyotrophic lateral sclerosis (ALS) and delivery methods for the treatment of spinal cord related disorders including ALS.

### RELATED APPLICATIONS

This application claims priority to U.S. Provisional Application No. 63/462,127 filed on Apr. 26, 2023, U.S. Provisional Application No. 63/545,873 filed on Oct. 26, 2023, and U.S. Provisional Application No. 63/564,293 filed on Mar. 12, 2024; the entire contents of each of which are hereby incorporated by reference in their entirety.

### SEQUENCE LISTING

The present application is being filed along with a Sequence Listing in electronic format. The Sequence Listing file, entitled V2071-3013PCT_SL.xml, was created on Apr. 10, 2024, and is 4,955,197 bytes in size. The information in electronic format of the Sequence Listing is incorporated herein by reference in its entirety.

### FIELD OF THE DISCLOSURE

The present disclosure relates to compositions, preparation, use, and/or formulation of adeno-associated virus (AAV) capsid proteins and variants thereof, for the delivery of a SOD1 targeting polynucleotide, e.g., small interfering RNA (siRNA) duplexes, shRNA, microRNA (miRNA), or precursors thereof which target or encode molecules which target the superoxide dismutase 1 (SOD1) gene to interfere with SOD1 gene expression and/or SOD1 enzyme production.

### BACKGROUND

Amyotrophic lateral sclerosis (ALS), also known as Lou Gehrig's disease, is a fatal progressive neurodegenerative disease, characterized by the predominant loss of upper and lower motor neurons (MNs) in primary motor cortex, the brainstem, and the spinal cord. Upper (e.g., cortical) and lower motor neurons (e.g., spinal cord) normally communicate messages from the brain to the muscles to generate voluntary movement. When these neurons degenerate and/or die, the loss of the message to the muscles results in a gradual weakening and/or atrophy of the muscle and inability to initiate or control voluntary movements, until ultimately, an individual suffering from ALS loses muscle strength and the ability to move, speak, cat and even breathe. Most patients will require some form of breathing aid for survival, and even then, most ALS patients die as a result of respiratory failure within 2-5 years of diagnosis. During disease progression, some patients (e.g., FTD-ALS) may also develop frontotemporal dementia.

Two forms of ALS have been described: one is sporadic ALS (sALS), which is the most common form of ALS in the United States of America and accounts for 90 to 95% of all cases diagnosed; the other is familial ALS (fALS), which occurs in a family lineage mainly with a dominant inheritance and only accounts for about 5 to 10% of all cases in the United States of America. sALS and fALS are clinically indistinguishable. Pathological studies have linked numerous cellular processes with disease pathogenesis such as increased ER stress, generation of free radicals (i.e., reactive oxygen species (ROS)), mitochondrial dysfunction, protein aggregation, apoptosis, inflammation and glutamate excitotoxicity, specifically in the motor neurons (MNs).

ALS is a complex genetic disorder in which multiple genes in combination with environmental exposures combine to render a person susceptible. More than a dozen genes associated with ALS have been discovered, including, SOD1 (Cu²⁺/Zn²⁺ superoxide dismutase), TDP-43 (TARDBP, TAR DNA binding protein-43), FUS (Fused in Sarcoma/Translocated in Sarcoma), ANG (Angiogenin), ATXN2 (Ataxin-2), valosin containing protein (VCP), OPTN (Optineurin) and an expansion of the noncoding GGGGCC hexanucleotide repeat in the chromosome 9, open reading frame 72 (C9ORF72). However, the exact mechanisms of motor neuron degeneration are still elusive.

Currently, there is no curative treatment for ALS. As such, there is a medical need for improved compositions and methods of prevention, treatment, and diagnosis for diseases associated with aberrant SOD1 expression, including ALS.

### SUMMARY

The present disclosure provides AAV particles encoding a SOD1 targeting polynucleotide to modulate, e.g., interfere with, SOD1 gene expression and/or SOD1 protein production and methods of use thereof. Methods for treating diseases associated with motor neuron degeneration such as amyotrophic lateral sclerosis (ALS) are also included in the present disclosure.

Accordingly, in one aspect, the present disclosure provides an AAV particle comprising an AAV capsid variant and a nucleic acid encoding a SOD1 targeting polynucleotide (e.g., a SOD1 targeting RNA agent, e.g., a siRNA duplex), wherein the AAV capsid variant comprises an amino acid sequence having the following formula: [N1]-[N2]-[N3], wherein: (i) optionally [N1] comprises X1, X2, and X3, wherein at least one of X1, X2, or X3 is G; (ii) [N2] comprises the amino acid sequence of SPH; and (iii) [N3] comprises X4, X5, and X6, wherein at least one of X4, X5, or X6 is a basic amino acid, e.g., a K or R. In some embodiments, position X4 of [N3] is K. In some embodiments, position X5 of [N3] is K. In some embodiments, [N3] is or comprises SKA. In some embodiments [N3] is or comprises KSG. In some embodiments, [N2]-[N3] is present immediately subsequent to position 455, numbered according to SEQ ID NO: 138, 981 or 982. In some embodiments, [N1] is present immediately subsequent to position 452, relative to a reference sequence numbered according to the amino acid sequence of SEQ ID NO: 138, 981, or 982. In some embodiments, [N1] replaces positions 453-455 (e.g., G453, S454, and G455), relative to a reference sequence numbered according to SEQ ID NO: 138. In some embodiments, the AAV capsid variant comprises H at position 454 and D at position 455, numbered according to SEQ ID NO: 138 or 982. In some embodiments, the AAV capsid variant comprises S at position 454 and G at position 455, numbered according to SEQ ID NO: 138 or 981. In some embodiments, an insert of 8 amino acids replaces the SG at positions 454-455, numbered according to SEQ ID NO: 138. In some embodiments, an insert of 6 amino acids is present immediately subsequent to position 455, numbered according to SEQ ID NO: 138, 981, or 982.

In yet another aspect, the present disclosure provides an AAV particle comprising an AAV capsid variant and a nucleic acid encoding a SOD1 targeting polynucleotide (e.g., a SOD1 targeting RNA agent, e.g., a siRNA duplex), wherein the AAV capsid variant comprises an amino acid sequence having the following formula: [N1]-[N2]-[N3] (SEQ ID NO: 3963), wherein: (i) [N1] comprises positions X1, X2, and X3, wherein position X2 is S and position X3 is G; (ii) [N2] comprises the amino acid sequence SPH; and (iii) [N3] comprises positions X4, X5, and X6, wherein position X5 is K. In some embodiments, [N1]-[N2]-[N3] is present immediately subsequent to position 452 and replaces positions 453-455, numbered according to SEQ ID NO: 138 or 982. In some embodiments, [N1]-[N2]-[N3] is or comprises GSGSPHSKA (SEQ ID NO: 1369).

In another aspect, the present disclosure provides an AAV particle comprising an AAV particle comprising an AAV capsid variant and a nucleic acid encoding a SOD1 targeting polynucleotide (e.g., a SOD1 targeting RNA agent, e.g., a siRNA duplex), wherein the AAV capsid variant comprises an amino acid sequence having the following formula: [N1]-[N2]-[N3] (SEQ ID NO: 4647), wherein: (i) [N1] comprises positions X1, X2, and X3, wherein position X2 is an amino acid other than S and position X3 is an amino acid other than G; (ii) [N2] comprises the amino acid sequence SPH; and (iii) [N3] comprises positions X4, X5, and X6, wherein position X4 is K. In some embodiments, [N1]-[N2]-[N3] is present immediately subsequent to position 452 and replaces positions 453-455, numbered according to SEQ ID NO: 138 or 982. In some embodiments, [N1]-[N2]-[N3] is or comprises GHDSPHKSG (SEQ ID NO: 1370).

In another aspect, the present disclosure provides an AAV particle comprising an AAV capsid variant and a nucleic acid encoding a SOD1 targeting polynucleotide (e.g., a SOD1 targeting RNA agent, e.g., a siRNA duplex), wherein the AAV capsid variant comprises (a) the amino acid sequence of any of the sequences provided in Tables 1A, 2A, 2B, 21-23, 25, 26, or 30; (b) an amino acid sequence comprising at least 3, 4, 5, 6, 7, 8, 9, 10, 11, 12, 13, 14, 15, 16, or 17, consecutive amino acids from any one of the sequences provided in Tables 1A, 2A, 2B, 21-23, 25, 26, or 30; (c) an amino acid sequence comprising at least one, two, or three, but no more than four different amino acids, relative to any one of the sequences provided in Tables 1A, 2A, 2B, 21-23, 25, 26, or 30; or (d) an amino acid sequence comprising at least one, two, or three but no more than four modifications, e.g., substitutions (e.g., conservative substitutions), relative to the amino acid sequence of any one of the sequences provided in Tables 1A, 2A, 2B, 21-23, 25, 26, or 30. In some embodiments, the amino acid sequence is present in loop IV. In some embodiments, the amino acid sequence is present immediately subsequent to position 448, 452, 453, 455, relative to a reference sequence numbered according to the amino acid sequence of SEQ ID NO: 138.

In yet another aspect, the present disclosure provides an AAV particle comprising an AAV capsid variant and a nucleic acid encoding a SOD1 targeting polynucleotide (e.g., a SOD1 targeting RNA agent, e.g., a siRNA duplex), wherein the AAV capsid variant comprises (a) the amino acid sequence of any of SEQ ID NOs: 945-980 or 985-986; (b) an amino acid sequence comprising at least 3, 4, or 5 consecutive amino acids from any one of SEQ ID NOs: 945-980 or 985-986; (c) an amino acid sequence comprising at least one, two, or three but no more than four different amino acids, relative to the amino acid sequence of any one of SEQ ID NOs: 945-980 or 985-986; (d) an amino acid sequence comprising at least one, two, or three but no more than four modifications, e.g., substitutions (e.g., conservative substitutions), relative to the amino acid sequence of any one of SEQ ID NOs: 945-980 or 985-986. In some embodiments, the amino acid sequence is present in loop IV. In some embodiments, the amino acid sequence is present immediately subsequent to position 448, 452, 453, 455, relative to a reference sequence numbered according to the amino acid sequence of SEQ ID NO: 138.

In yet another aspect, the present disclosure provides an AAV particle comprising an AAV capsid variant and a nucleic acid encoding a SOD1 targeting polynucleotide (e.g., a SOD1 targeting RNA agent, e.g., a siRNA duplex), wherein the AAV capsid variant comprises (a) the amino acid sequence of any of SEQ ID NOs: 2, 200, 201, 941, 943, 204, 208, 404, or 903-909; (b) an amino acid sequence comprising at least 3, 4, 5, 6, 7, 8, 9, 10, 11, 12, or 13 consecutive amino acids from any one of SEQ ID NOs: 2, 200, 201, 941, 943, 204, 208, 404, or 903-909; (c) an amino acid sequence comprising at least one, two, or three, but no more than four different amino acids, relative to the amino acid sequence of any one of SEQ ID NOs: 2, 200, 201, 941, 943, 204, 208, 404, or 903-909; or (d) an amino acid sequence comprising at least one, two, or three but no more than four modifications, e.g., substitutions (e.g., conservative substitutions), relative to the amino acid sequence of any one of SEQ ID NOs: 2, 200, 201, 941, 943, 204, 208, 404, or 903-909. In some embodiments, the amino acid sequence is present in loop IV. In some embodiments, the amino acid sequence is present immediately subsequent to position 448, 452, 453, 455, relative to a reference sequence numbered according to the amino acid sequence of SEQ ID NO: 138.

In yet another aspect, the present disclosure provides an AAV particle comprising an AAV capsid variant and a nucleic acid encoding a SOD1 targeting polynucleotide (e.g., a SOD1 targeting RNA agent, e.g., a siRNA duplex), wherein the AAV capsid variant comprises the amino acid sequence of SPH, wherein the amino acid sequence is present immediately subsequent to position 455, numbered according to the amino acid sequence of any one of SEQ ID NOs: 69, 70, 71, 4, 5, 6, 7, 8, 36-59, 138, 981, or 982.

In yet another aspect, present disclosure provides an AAV particle comprising an AAV capsid variant and a nucleic acid encoding a SOD1 targeting polynucleotide (e.g., a SOD1 targeting RNA agent, e.g., a siRNA duplex), wherein the AAV capsid variant comprises the amino acid sequence of SPHSKA (SEQ ID NO: 941), wherein the amino acid sequence is present immediately subsequent to position 455, numbered according to the amino acid sequence of SEQ ID NO: 138.

In yet another aspect, present disclosure provides an AAV particle comprising an AAV capsid agent, e.g., a siRNA duplex), wherein the AAV capsid variant comprises the amino acid sequence of SPHSKA (SEQ ID NO: 941), wherein the amino acid sequence is present immediately subsequent to position 455, numbered according to the amino acid sequence of SEQ ID NO: 981.

In yet another aspect, present disclosure provides an AAV particle comprising an AAV capsid variant and a nucleic acid encoding a SOD1 targeting polynucleotide (e.g., a SOD1 targeting RNA agent, e.g., a siRNA duplex), wherein the AAV capsid variant comprises the amino acid sequence of HDSPHK (SEQ ID NO: 2), wherein the amino acid sequence is present immediately subsequent to position 453, numbered according to the amino acid sequence of SEQ ID NO: 138.

In yet another aspect, present disclosure provides an AAV particle comprising an AAV capsid variant and a nucleic acid encoding a SOD1 targeting polynucleotide (e.g., a SOD1 targeting RNA agent, e.g., a siRNA duplex), wherein the AAV capsid variant comprises the amino acid sequence of HDSPHK (SEQ ID NO: 2), wherein the amino acid sequence is present immediately subsequent to position 453, numbered according to the amino acid sequence of SEQ ID NO: 982.

In yet another aspect, the present disclosure provides an AAV particle comprising an AAV capsid variant and a nucleic acid encoding a polynucleotide that targets human SOD1, wherein the AAV capsid variant comprises an amino acid sequence having the following formula: [N1]-[N2]-[N3], wherein: (i) [N1] comprises X1, X2, and X3, wherein at least one of X1, X2, or X3 is G; (ii) [N2] comprises the amino acid sequence of SPH; and (iii) [N3] comprises X4, X5, and X6, wherein at least one of X4, X5, or X6 is a basic amino acid, e.g., a K or R; wherein [N1]-[N2]-[N3] is present in hypervariable loop IV; and wherein the AAV capsid variant comprises an amino acid sequence at least 95% identical to the amino acid sequence of positions 203-736 of SEQ ID NO: 138.

In yet another aspect, the present disclosure provides an AAV particle comprising an AAV capsid variant and a nucleic acid encoding a polynucleotide that targets human SOD1, wherein the AAV capsid variant comprises the amino acid sequence of SPHSKA (SEQ ID NO: 941) in hypervariable loop IV. In some embodiments, hypervariable loop IV comprises amino acids 449-460, numbered according to SEQ ID NO: 138. In some embodiments, the amino acid sequence of SEQ ID NO: 941 is present immediately subsequent to position 455, numbered according to SEQ ID NO: 981. In some embodiments, the AAV capsid variant comprises the amino acid E at position 451, numbered according to SEQ ID NO: 981. In some embodiments, the AAV capsid variant comprises the amino acid V at position 453, numbered according to SEQ ID NO: 981. In some embodiments, the AAV capsid variant comprises the amino acid sequence of SPHSKA (SEQ ID NO: 941); the amino acid E at position 451, numbered according to SEQ ID NO: 981; and the amino acid V at position 453, numbered according to SEQ ID NO: 69 or 981.

In yet another aspect, the present disclosure provides an AAV particle comprising an AAV capsid variant and a nucleic acid encoding a polynucleotide that targets human SOD1, wherein the AAV capsid variant comprises the amino acid sequence of SPHSKA (SEQ ID NO: 941) in hypervariable loop IV. In some embodiments, hypervariable loop IV comprises amino acids 449-460, numbered according to SEQ ID NO: 138. In some embodiments, the amino acid sequence of SEQ ID NO: 941 is present immediately subsequent to position 455, numbered according to SEQ ID NO: 981. In some embodiments, the AAV capsid variant comprises the amino acid E at position 451, numbered according to SEQ ID NO: 981. In some embodiments, the AAV capsid variant comprises the amino acid R at position 452, numbered according to SEQ ID NO: 981. In some embodiments, the AAV capsid variant comprises the amino acid V at position 453, numbered according to SEQ ID NO: 981. In some embodiments, the AAV capsid variant comprises the amino acid sequence of SPHSKA (SEQ ID NO: 941); the amino acid E at position 451, numbered according to SEQ ID NO: 981; the amino acid R at position 452, numbered according to SEQ ID NO: 981; and the amino acid V at position 453, numbered according to SEQ ID NO: 36 or 981.

In yet another aspect, the present disclosure provides an AAV particle comprising: (i) an AAV capsid variant comprising the amino acid sequence of SEQ ID NO: 69; and (ii) a nucleic acid encoding a polynucleotide targeting human SOD1, wherein the encoded polynucleotide comprises a siRNA comprising a sense strand sequence and an antisense strand sequence, wherein the encoded sense strand sequence comprises SEQ ID NO: 2525, and the encoded antisense strand sequence comprises SEQ ID NO: 2507.

In yet another aspect, the present disclosure provides an AAV particle comprising: (i) an AAV capsid variant comprising the amino acid sequence of SEQ ID NO: 36; and (ii) a nucleic acid encoding a polynucleotide targeting human SOD1, wherein the encoded polynucleotide comprises a siRNA comprising a sense strand sequence and an antisense strand sequence, wherein the encoded sense strand sequence comprises SEQ ID NO: 2525, and the encoded antisense strand sequence comprises SEQ ID NO: 2507.

In yet another aspect, the present disclosure provides an AAV particle comprising: (i) an AAV capsid variant comprising the amino acid sequence of SEQ ID NO: 982; and (ii) a nucleic acid encoding a polynucleotide targeting human SOD1, wherein the encoded polynucleotide comprises a siRNA comprising a sense strand sequence and an antisense strand sequence, wherein the encoded sense strand sequence comprises SEQ ID NO: 2525, and the encoded antisense strand sequence comprises SEQ ID NO: 2507.

In yet another aspect, the present disclosure provides an AAV particle comprising: (i) an AAV capsid variant comprising the amino acid sequence of SEQ ID NO: 981; and (ii) a nucleic acid encoding a polynucleotide targeting human SOD1, wherein the encoded polynucleotide comprises a siRNA comprising a sense strand sequence and an antisense strand sequence, wherein the encoded sense strand sequence comprises SEQ ID NO: 2525, and the encoded antisense strand sequence comprises SEQ ID NO: 2507.

In yet another aspect, the present disclosure provides an AAV particle comprising: (i) an AAV capsid variant comprising the amino acid sequence of SEQ ID NO: 69; and (ii) a nucleic acid encoding a polynucleotide targeting human SOD1, wherein the encoded polynucleotide comprises the nucleotide sequence of SEQ ID NO: 5013.

In yet another aspect, the present disclosure provides an AAV particle comprising: (i) an AAV capsid variant comprising the amino acid sequence of SEQ ID NO: 36; and (ii) a nucleic acid encoding a polynucleotide targeting human SOD1, wherein the encoded polynucleotide comprises the nucleotide sequence of SEQ ID NO: 5013.

In yet another aspect, the present disclosure provides an AAV particle comprising: (i) an AAV capsid variant comprising the amino acid sequence of SEQ ID NO: 982; and (ii) a nucleic acid encoding a polynucleotide targeting human SOD1, wherein the encoded polynucleotide comprises the nucleotide sequence of SEQ ID NO: 5013.

In yet another aspect, the present disclosure provides an AAV particle comprising: (i) an AAV capsid variant comprising the amino acid sequence of SEQ ID NO: 981; and (ii) a nucleic acid encoding a polynucleotide targeting human SOD1, wherein the encoded polynucleotide comprises the nucleotide sequence of SEQ ID NO: 5013.

In yet another aspect, the present disclosure provides an AAV particle comprising: (i) an AAV capsid variant comprising the amino acid sequence of SEQ ID NO: 69; and (ii) a nucleic acid encoding a polynucleotide targeting human SOD1, wherein the polynucleotide comprises the nucleotide sequence of SEQ ID NO: 2562.

In yet another aspect, the present disclosure provides an AAV particle comprising: (i) an AAV capsid variant comprising the amino acid sequence of SEQ ID NO: 36; and (ii) a nucleic acid encoding a polynucleotide targeting human SOD1, wherein the polynucleotide comprises the nucleotide sequence of SEQ ID NO: 2562.

In yet another aspect, the present disclosure provides an AAV particle comprising: (i) an AAV capsid variant comprising the amino acid sequence of SEQ ID NO: 982; and (ii) a nucleic acid encoding a polynucleotide targeting human SOD1, wherein the polynucleotide comprises the nucleotide sequence of SEQ ID NO: 2562.

In yet another aspect, the present disclosure provides an AAV particle comprising: (i) an AAV capsid variant comprising the amino acid sequence of SEQ ID NO: 981; and (ii) a nucleic acid encoding a polynucleotide targeting human SOD1, wherein the polynucleotide comprises the nucleotide sequence of SEQ ID NO: 2562.

In yet another aspect, the present disclosure provides an AAV particle comprising: (i) an AAV capsid variant comprising the amino acid sequence of SEQ ID NO: 69; and (ii) a viral genome comprising the nucleotide sequence of SEQ ID NO: 109.

In yet another aspect, the present disclosure provides an AAV particle comprising: (i) an AAV capsid variant comprising the amino acid sequence of SEQ ID NO: 36; and (ii) a viral genome comprising the nucleotide sequence of SEQ ID NO: 109.

In yet another aspect, the present disclosure provides an AAV particle comprising: (i) an AAV capsid variant comprising the amino acid sequence of SEQ ID NO: 982; and (ii) a viral genome comprising the nucleotide sequence of SEQ ID NO: 109.

In yet another aspect, the present disclosure provides an AAV particle comprising: (i) an AAV capsid variant comprising the amino acid sequence of SEQ ID NO: 981; and (ii) a nucleic acid encoding a polynucleotide targeting human SOD1, wherein the encoded polynucleotide comprises the nucleotide sequence of SEQ ID NO: 5013.

In yet another aspect, present disclosure provides an AAV particle comprising an AAV capsid variant and a nucleic acid encoding a SOD1 targeting polynucleotide (e.g., a SOD1 targeting RNA agent, e.g., a siRNA duplex), wherein the AAV capsid variant comprises an amino acid sequence comprising at least 3, 4, 5, or 6 consecutive amino acids from the amino acid sequence of SPHSKA (SEQ ID NO: 941), wherein: (i) the at least 3 consecutive amino acids comprise SPH; (ii) the at least 4 consecutive amino acids comprise SPHS (SEQ ID NO: 1371); (iii) the at least 5 consecutive amino acids comprise SPHSK (SEQ ID NO: 1372); or (iv) the at least 6 consecutive amino acids comprise SPHSKA (SEQ ID NO: 941); wherein the AAV capsid variant comprises: (a) a VP1 protein comprising the amino acid sequence of SEQ ID NO: 138 or SEQ ID NO: 981; (b) a VP2 protein comprising the amino acid sequence of positions 138-736 of SEQ ID NO: 138 or positions 138-742 of SEQ ID NO: 981; (c) a VP3 protein comprising the amino acid sequence of positions 203-736 of SEQ ID NO: 138 or positions 203-742 of SEQ ID NO: 981; or (d) an amino acid sequence with at least 90% (e.g., at least about 95, 96, 97, 98, or 99%) sequence identity to any of the amino acid sequences in (a)-(c). In some embodiments, the amino acid sequence is present immediately subsequent to positions 455, numbered according to SEQ ID NO: 138 or 981.

In yet another aspect, the present disclosure provides an AAV particle comprising an AAV capsid variant and a nucleic acid encoding a SOD1 targeting polynucleotide (e.g., a SOD1 targeting RNA agent, e.g., a siRNA duplex), wherein the AAV capsid variant comprises one or two, but no more than three substitutions relative to the amino acid sequence of SPHSKA (SEQ ID NO: 941), wherein the AAV capsid variant comprises: (a) a VP1 protein comprising the amino acid sequence of SEQ ID NO: 138 or SEQ ID NO: 981; (b) a VP2 protein comprising the amino acid sequence of positions 138-736 of SEQ ID NO: 138 or positions 138-742 of SEQ ID NO: 981; (c) a VP3 protein comprising the amino acid sequence of positions 203-736 of SEQ ID NO: 138 or positions 203-742 of SEQ ID NO: 981; or (d) an amino acid sequence with at least 90% (e.g., at least about 95, 96, 97, 98, or 99%) sequence identity to any of the amino acid sequences in (a)-(c). In some embodiments, the amino acid sequence is present immediately subsequent to positions 455, numbered according to SEQ ID NO: 138 or 981.

In another aspect, the present disclosure provides an AAV particle comprising an AAV capsid agent, e.g., a siRNA duplex), wherein the AAV capsid variant comprises at least 3, 4, 5, or 6 consecutive amino acids from the amino acid sequence of HDSPHK (SEQ ID NO: 2), wherein: (i) the at least 3 consecutive amino acids comprise HDS; (ii) the at least 4 consecutive amino acids comprise HDSP (SEQ ID NO: 1373); (iii) the at least 5 consecutive amino acids comprise HDSPH (SEQ ID NO: 1374); and/or (iv) the at least 6 consecutive amino acids comprise HDSPHK (SEQ ID NO: 2); wherein the AAV capsid variant comprises: (a) a VP1 protein comprising the amino acid sequence of SEQ ID NO: 138 or SEQ ID NO: 982; (b) a VP2 protein comprising the amino acid sequence of positions 138-736 of SEQ ID NO: 138 or positions 138-742 of SEQ ID NO: 982; (c) a VP3 protein comprising the amino acid sequence of positions 203-736 of SEQ ID NO: 138 or positions 203-742 of SEQ ID NO: 982; or (d) an amino acid sequence with at least 90% (e.g., at least about 95, 96, 97, 98, or 99%) sequence identity to any of the amino acid sequences in (a)-(c). In some embodiments, the amino acid sequence is present immediately subsequent to positions 453, numbered according to SEQ ID NO: 138 or 982.

In another aspect, the present disclosure provides an AAV particle comprising an AAV capsid variant and a nucleic acid encoding a SOD1 targeting polynucleotide (e.g., a SOD1 targeting RNA agent, e.g., a siRNA duplex), wherein the AAV capsid variant comprises one or two, but no more than three substitutions relative to the amino acid sequence of HDSPHK (SEQ ID NO: 2), wherein the AAV capsid variant comprises: (a) a VP1 protein comprising the amino acid sequence of SEQ ID NO: 138 or SEQ ID NO: 982; (b) a VP2 protein comprising the amino acid sequence of positions 138-736 of SEQ ID NO: 138 or positions 138-742 of SEQ ID NO: 982; (c) a VP3 protein comprising the amino acid sequence of positions 203-736 of SEQ ID NO: 138 or positions 203-742 of SEQ ID NO: 982; or (d) an amino acid sequence with at least 90% (e.g., at least about 95, 96, 97, 98, or 99%) sequence identity to any of the amino acid sequences in (a)-(c). In some embodiments, the amino acid sequence is present immediately subsequent to positions 453, numbered according to SEQ ID NO: 138 or 982.

In accordance with some aspects of the present disclosure the SOD1 targeting polynucleotide, e.g., the SOD1 targeting RNA agent, is a single-stranded antisense RNA molecule, a single-stranded siRNA, or a double-stranded RNA (e.g., a siRNA duplex) that inhibits expression of SOD1. For example, the SOD1 targeting polynucleotide may be a siRNA duplex comprising: (i) a sense strand sequence comprising at least 15 (e.g., at least 16, 17, 18, 19, 20, 21, 22, or 23) contiguous nucleotides differing by no more than 3 (e.g., by no more than 0, 1 or 2) nucleotides from a sense sequence listed in Table 10 or 14; and (ii) an antisense strand sequence comprising at least 15 (e.g., at least 16, 17, 18, 19, 20, 21, 22, or 23) contiguous nucleotides differing by no more than 3 (e.g., by no more than 0, 1 or 2) nucleotides from an antisense sequence in Table 10 or 14.

In yet another aspect, the present disclosure provides a cell, e.g., a host cell, comprising the AAV particles described herein. The method of making the AAV particle may comprise: (i) providing a host cell comprising a viral genome; and (ii) incubating the host cell under conditions suitable to enclose the viral genome in an AAV capsid variant, e.g., an AAV capsid variant described herein; thereby making the AAV particle.

Further disclosed are pharmaceutical compositions including the AAV particles as described herein, and a pharmaceutically acceptable excipient. The administration of the pharmaceutical compositions may be used in the methods of treating a subject having or diagnosed with having a neurological disorder, e.g., a neurodegenerative disorder. In some embodiments the neurological disorder, neurodegenerative disorder, or disease associated with SOD1 expression is amyotrophic lateral sclerosis (ALS). In some embodiments, the AAV particles are administered intravenously or by ICM, or a combination thereof.

In some aspects, ALS is familial ALS linked to SOD1 mutations. In other aspects, ALS is sporadic ALS which is characterized by abnormal aggregation of SOD1 protein or disruption of SOD1 protein function or localization, though not necessarily as a result of genetic mutation. The symptoms of ALS ameliorated by the present method may include motor neuron degeneration, muscle weakness, stiffness of muscles, slurred speech and/or difficulty in breathing. The ALS may be early stage ALS, middle stage ALS; and/or late stage ALS.

Those skilled in the art will recognize or be able to ascertain using no more than routine experimentation, many equivalents to the specific embodiments of the invention described herein. Such equivalents are intended to be encompassed by the following enumerated embodiments.

### Enumerated Embodiments

1. An AAV particle comprising an AAV capsid variant and a nucleic acid encoding a SOD1 (e.g., human SOD1) targeting polynucleotide (e.g., a SOD1 targeting RNA agent, e.g., a siRNA), wherein the AAV capsid variant comprises an amino acid sequence having the following formula: [N1]-[N2]-[N3], wherein:
   (i) optionally [N1] comprises X1, X2, and X3, wherein at least one of X1, X2, or X3 is G;
   (ii) [N2] comprises the amino acid sequence of SPH; and
   (iii) [N3] comprises X4, X5, and X6, wherein at least one of X4, X5, or X6 is a basic amino acid, e.g., a K or R.
2. The AAV particle of embodiment 1, wherein X4, X5, or both of [N3] is a K.
3. The AAV particle of embodiment 1 or 2, wherein X4, X5, or X6 of [N3] is an R.
4. The AAV particle of any one of embodiments 1-3, wherein:
   (a) position X4 of [N3] is: K, S, A, V, T, G, F, W, V, N, or R;
   (b) position X5 of [N3] is: S, K, T, F, I, L, Y, H, M, or R; and/or
   (c) position X6 of [N3] is: G, A, R, M, I, N, T, Y, D, P, V, L, E, W, N, Q, K, or S;
   optionally wherein the AAV capsid variant comprises an amino acid modification, e.g., a conservative substitution, of any of the aforesaid amino acids in (a)-(c).
5. The AAV particle of any one of embodiments 1-4, wherein [N3] comprises SK, KA, KS, AR, RM, VK, AS, SR, VK, KR, KK, KN, VR, RS, RK, KT, TS, KF, FG, KI, IG, KL, LG, TT, TY, KY, YG, KD, KP, TR, RG, VR, GA, SL, SS, FL, WK, SA, RA, LR, KW, RR, GK, TK, NK, AK, KV, KG, KH, KM, TG, SE, SV, SW, SN, HG, SQ, LW, MG, MA, or SG.
6. The AAV particle of any one of embodiments 1-5, wherein [N3] is or comprises SKA, KSG, ARM, VKS, ASR, VKI, KKN, VRM, RKA, KTS, KFG, KIG, KLG, KTT, KTY, KYG, SKD, SKP, TRG, VRG, KRG, GAR, KSA, KSR, SKL, SRA, SKR, SLR, SRG, SSR, FLR, SKW, SKS, WKA, VRR, SKV, SKT, SKG, GKA, TKA, NKA, SKL, SKN, AKA, KTG, KSL, KSE, KSV, KSW, KSN, KHG, KSQ, KSK, KLW, WKG, KMG, KMA, or RSG.
7. The AAV particle of any one of embodiments 1-6, wherein [N2]-[N3] comprises SPHSK (SEQ ID NO: 1372), SPHKS (SEQ ID NO: 1375), SPHAR (SEQ ID NO: 1376), SPHVK (SEQ ID NO: 1377), SPHAS (SEQ ID NO: 1378), SPHKK (SEQ ID NO: 1379), SPHVR (SEQ ID NO: 1380), SPHRK (SEQ ID NO: 1381), SPHKT (SEQ ID NO: 1382), SPHKF (SEQ ID NO: 1383), SPHKI (SEQ ID NO: 1384), SPHKL (SEQ ID NO: 1385), SPHKY (SEQ ID NO: 1386), SPHTR (SEQ ID NO: 1387), SPHKR (SEQ ID NO: 1388), SPHGA (SEQ ID NO: 1389), SPHSR (SEQ ID NO: 1390), SPHSL (SEQ ID NO: 1391), SPHSS (SEQ ID NO: 1392), SPHFL (SEQ ID NO: 1393), SPHWK (SEQ ID NO: 1394), SPHGK (SEQ ID NO: 1395), SPHTK (SEQ ID NO: 1396), SPHNK (SEQ ID NO: 1397), SPHAK (SEQ ID NO: 1398), SPHKH (SEQ ID NO: 1399), SPHKM (SEQ ID NO: 1400), or SPHRS (SEQ ID NO: 1401).
8. The AAV particle of any one of embodiments 1-7, wherein [N2]-[N3] is or comprises:

(i)
(SEQ ID NO: 941)
SPHSKA,
(SEQ ID NO: 946)
SPHKSG,
(SEQ ID NO: 947)
SPHARM,
(SEQ ID NO: 948)
SPHVKS,
(SEQ ID NO: 949)
SPHASR,
(SEQ ID NO: 950)
SPHVKI,
(SEQ ID NO: 954)
SPHKKN,
(SEQ ID NO: 955)
SPHVRM,
(SEQ ID NO: 956)
SPHRKA,
(SEQ ID NO: 957)
SPHKFG,
(SEQ ID NO: 958)
SPHKIG,
(SEQ ID NO: 959)
SPHKLG,
(SEQ ID NO: 963)
SPHKTS,
(SEQ ID NO: 964)
SPHKTT,
(SEQ ID NO: 965)
SPHKTY,
(SEQ ID NO: 966)
SPHKYG,
(SEQ ID NO: 967)
SPHSKD,
(SEQ ID NO: 968)
SPHSKP,
(SEQ ID NO: 972)
SPHTRG,
(SEQ ID NO: 973)
SPHVRG,
(SEQ ID NO: 974)
SPHKRG,
(SEQ ID NO: 975)
SPHGAR,
(SEQ ID NO: 977)
SPHKSA,
(SEQ ID NO: 951)
SPHKSR,
(SEQ ID NO: 960)
SPHSKL,
(SEQ ID NO: 969)
SPHSRA,
(SEQ ID NO: 978)
SPHSKR,
(SEQ ID NO: 952)
SPHSLR,
(SEQ ID NO: 961)
SPHSRG,
(SEQ ID NO: 970)
SPHSSR,
(SEQ ID NO: 979)
SPHFLR,
(SEQ ID NO: 953)
SPHSKW,
(SEQ ID NO: 1402)
SPHSKS,
(SEQ ID NO: 971)
SPHWKA,
(SEQ ID NO: 980)
SPHVRR,
(SEQ ID NO: 1403)
SPHSKT,
(SEQ ID NO: 1404)
SPHSKG,
(SEQ ID NO: 1405)
SPHGKA,
(SEQ ID NO: 1406)
SPHNKA,
(SEQ ID NO: 1407)
SPHSKN,
(SEQ ID NO: 1408)
SPHAKA,
(SEQ ID NO: 1409)
SPHSKV,
(SEQ ID NO: 1410)
SPHKTG,
(SEQ ID NO: 1411)
SPHTKA,
(SEQ ID NO: 1412)
SPHKSL,
(SEQ ID NO: 1413)
SPHKSE,
(SEQ ID NO: 1414)
SPHKSV,
(SEQ ID NO: 1415)
SPHKSW,
(SEQ ID NO: 1416)
SPHKSN,
(SEQ ID NO: 1417)
SPHKHG,
(SEQ ID NO: 1418)
SPHKSQ,
(SEQ ID NO: 1419)
SPHKSK,
(SEQ ID NO: 1420)
SPHKLW,
(SEQ ID NO: 1421)
SPHWKG,
(SEQ ID NO: 1422)
SPHKMG,
(SEQ ID NO: 1423)
SPHKMA,
or
(SEQ ID NO: 976)
SPHRSG;

(ii) an amino acid sequence comprising any portion of an amino acid sequence in (i), e.g., any 2, 3, 4, or 5 amino acids, e.g., consecutive amino acids, thereof;
(iii) an amino acid sequence comprising one, two, or three but no more than four modifications, e.g., substitutions (e.g., conservative substitutions), insertions, or deletions, relative to any of the amino acid sequences in (i); or
(iv) an amino acid sequence comprising one, two, or three but no more than four different amino acids, relative to any one of the amino acid sequences in (i).
9. The AAV particle of any one of embodiments 1-8, wherein the AAV capsid variant comprises an amino acid other than G at position 453 (e.g., V, R, D, E, M, T, I, S, A, N, L, K, H, P, W, or C), an amino acid other than S at position 454 (V, L, N, D, H, R, P, G, T, I, A, E, Y, M, or Q), and/or a G at position 455 (e.g., C, L, D, E, Y, H, V, A, N, P, or S), numbered according to any one of SEQ ID NOs: 69, 70, 71, 4, 5, 6, 7, 8, 36-59, 138, 981 or 982.
10. The AAV particle of any one of embodiments 1-8, wherein the AAV capsid variant comprises the amino acid G at position 453, the amino acid S at position 454, and the amino acid G at position 455, numbered according to SEQ ID NO: 138 or 981.
11. The AAV particle of any one of embodiments 1-9, wherein the AAV capsid variant comprises the amino acid G at position 453, the amino acid H at position 454, and the amino acid D at position 455, numbered according to SEQ ID NO: 138 or 982.
12. The AAV particle of any one of embodiments 1-11, wherein [N1] comprises X1, X2, and X3, wherein at least one of X1, X2, or X3 is G.
13. The AAV particle of any one of embodiments 1-12, wherein:
   (a) position X1 of [N1] is: G, V, R, D, E, M, T, I, S, A, N, L, K, H, P, W, or C;
   (b) position X2 of [N1] is: S, V, L, N, D, H, R, P, G, T, I, A, E, Y, M, or Q; and/or
   (c) position X3 of [N1] is: G, C, L, D, E, Y, H, V, A, N, P, or S;
   optionally wherein the AAV capsid variant comprises an amino acid modification, e.g., a conservative substitution, of any of the aforesaid amino acids in (a)-(c).
14. The AAV particle of any one of embodiments 1-13, wherein [N1] comprises GS, SG, GH, HD, GQ, QD, VS, CS, GR, RG, QS, SH, MS, RN, TS, IS, GP, ES, SS, GN, AS, NS, LS, GG, KS, GT, PS, RS, GI, WS, DS, ID, GL, DA, DG, ME, EN, KN, KE, AI, NG, PG, TG, SV, IG, LG, AG, EG, SA, YD, HE, HG, RD, ND, PD, MG, QV, DD, HN, HP, GY, GM, GD, or HS.
15. The AAV particle of any one of embodiments 1-14, wherein [N1] is or comprises GSG, GHD, GQD, VSG, CSG, GRG, CSH, GQS, GSH, RVG, GSC, GLL, GDD, GHE, GNY, MSG, RNG, TSG, ISG, GPG, ESG, SSG, GNG, ASG, NSG, LSG, GGG, KSG, HSG, GTG, PSG, GSV, RSG, GIG, WSG, DSG, IDG, GLG, DAG, DGG, MEG, ENG, GSA, KNG, KEG, AIG, GYD, GHG, GRD, GND, GPD, GMG, GQV, GHN, GHP, or GHS.
16. The AAV particle of any one of embodiments 1-15, wherein [N1]-[N2] comprises

(i)
(SEQ ID NO: 1424)
SGSPH,
(SEQ ID NO: 1374)
HDSPH,
(SEQ ID NO: 1432)
VGSPH,
(SEQ ID NO: 1425)
QDSPH,
(SEQ ID NO: 1426)
RGSPH,
(SEQ ID NO: 1427)
SHSPH,
(SEQ ID NO: 1428)
QSSPH,
(SEQ ID NO: 1429)
DDSPH,
(SEQ ID NO: 1430)
HESPH,
(SEQ ID NO: 1431)
NYSPH,
(SEQ ID NO: 1433)
SCSPH,
(SEQ ID NO: 1434)
LLSPH,
(SEQ ID NO: 1435)
NGSPH,
(SEQ ID NO: 1436)
PGSPH,
(SEQ ID NO: 1437)
GGSPH,
(SEQ ID NO: 1438)
TGSPH,
(SEQ ID NO: 1439)
SVSPH,
(SEQ ID NO: 1440)
IGSPH,
(SEQ ID NO: 1441)
DGSPH,
(SEQ ID NO: 1442)
LGSPH,
(SEQ ID NO: 1443)
AGSPH,
(SEQ ID NO: 1444)
EGSPH,
(SEQ ID NO: 1445)
SASPH,
(SEQ ID NO: 1446)
YDSPH,
(SEQ ID NO: 1447)
HGSPH,
(SEQ ID NO: 1448)
RDSPH,
(SEQ ID NO: 1449)
NDSPH,
(SEQ ID NO: 1450)
PDSPH,
(SEQ ID NO: 1451)
MGSPH,
(SEQ ID NO: 1452)
QVSPH,
(SEQ ID NO: 1453)
HNSPH,
(SEQ ID NO: 1454)
HPSPH,
or
(SEQ ID NO: 1455)
HSSPH;

(ii) an amino acid sequence comprising any portion of an amino acid sequence in (i), e.g., any 2, 3, or 4 amino acids, e.g., consecutive amino acids, thereof;
(iii) an amino acid sequence comprising one, two, or three but no more than four modifications, e.g., substitutions (e.g., conservative substitutions), insertions, or deletions, relative to any of the amino acid sequences in (i); or
(iv) an amino acid sequence comprising one, two, or three but no more than four different amino acids, relative to any one of the amino acid sequences in (i).
17. The AAV particle of any one of embodiments 1-16, wherein [N1]-[N2] is or comprises:

(i)
(SEQ ID NO: 1456)
GSGSPH,
(SEQ ID NO: 1457)
GHDSPH,
(SEQ ID NO: 1459)
VSGSPH,
(SEQ ID NO: 1458)
GQDSPH,
(SEQ ID NO: 1460)
CSGSPH,
(SEQ ID NO: 1461)
GRGSPH,
(SEQ ID NO: 1462)
CSHSPH,
(SEQ ID NO: 1463)
GQSSPH,
(SEQ ID NO: 1464)
GSHSPH,
(SEQ ID NO: 1465)
GDDSPH,
(SEQ ID NO: 1466)
GHESPH,
(SEQ ID NO: 1467)
GNYSPH,
(SEQ ID NO: 1468)
RVGSPH,
(SEQ ID NO: 1469)
GSCSPH,
(SEQ ID NO: 1470)
GLLSPH,
(SEQ ID NO: 1471)
MSGSPH,
(SEQ ID NO: 1472)
RNGSPH,
(SEQ ID NO: 1473)
TSGSPH,
(SEQ ID NO: 1474)
ISGSPH,
(SEQ ID NO: 1475)
GPGSPH,
(SEQ ID NO: 1476)
ESGSPH,
(SEQ ID NO: 1477)
SSGSPH,
(SEQ ID NO: 1478)
GNGSPH,
(SEQ ID NO: 1479)
ASGSPH,
(SEQ ID NO: 1480)
NSGSPH,
(SEQ ID NO: 1481)
LSGSPH,
(SEQ ID NO: 1482)
GGGSPH,
(SEQ ID NO: 1483)
KSGSPH,
(SEQ ID NO: 1484)
HSGSPH,
(SEQ ID NO: 1485)
GTGSPH,
(SEQ ID NO: 1486)
PSGSPH,
(SEQ ID NO: 1487)
GSVSPH,
(SEQ ID NO: 1488)
RSGSPH,
(SEQ ID NO: 1489)
GIGSPH,
(SEQ ID NO: 1490)
WSGSPH,
(SEQ ID NO: 1491)
DSGSPH,
(SEQ ID NO: 1492)
IDGSPH,
(SEQ ID NO: 1493)
GLGSPH,
(SEQ ID NO: 1494)
DAGSPH,
(SEQ ID NO: 1495)
DGGSPH,
(SEQ ID NO: 1496)
MEGSPH,
(SEQ ID NO: 1497)
ENGSPH,
(SEQ ID NO: 1498)
GSASPH,
(SEQ ID NO: 1499)
KNGSPH,
(SEQ ID NO: 1500)
KEGSPH,
(SEQ ID NO: 1501)
AIGSPH,
(SEQ ID NO: 1502)
GYDSPH,
(SEQ ID NO: 1503)
GHGSPH,
(SEQ ID NO: 1504)
GRDSPH,
(SEQ ID NO: 1505)
GNDSPH,
(SEQ ID NO: 1506)
GPDSPH,
(SEQ ID NO: 1507)
GMGSPH,
(SEQ ID NO: 1508)
GQVSPH,
(SEQ ID NO: 1509)
GHNSPH,
(SEQ ID NO: 1510)
GHPSPH,
or
(SEQ ID NO: 1511)
GHSSPH;

(ii) an amino acid sequence comprising any portion of an amino acid sequence in (i), e.g., any 2, 3, 4, or 5 amino acids, e.g., consecutive amino acids, thereof;
(iii) an amino acid sequence comprising one, two, or three but no more than four modifications, e.g., substitutions (e.g., conservative substitutions), insertions, or deletions, relative to any of the amino acid sequences in (i); or
(iv) an amino acid sequence comprising one, two, or three but no more than four different amino acids, relative to any one of the amino acid sequences in (i).
18. The AAV particle of any one of embodiments 1-17, wherein [N1]-[N2]-[N3] comprises:

(i)
(SEQ ID NO: 1512)
SGSPHSK,
(SEQ ID NO: 1513)
HDSPHKS,
(SEQ ID NO: 1540)
VGSPHSK,
(SEQ ID NO: 1514)
SGSPHAR,
(SEQ ID NO: 1515)
SGSPHVK,
(SEQ ID NO: 1516)
QDSPHKS,
(SEQ ID NO: 1517)
SGSPHKK,
(SEQ ID NO: 1518)
SGSPHVR,
(SEQ ID NO: 1519)
SGSPHAS,
(SEQ ID NO: 1520)
SGSPHRK,
(SEQ ID NO: 1521)
SGSPHKT,
(SEQ ID NO: 1522)
SHSPHKS,
(SEQ ID NO: 1523)
QSSPHRS,
(SEQ ID NO: 1524)
RGSPHAS,
(SEQ ID NO: 1525)
RGSPHSK,
(SEQ ID NO: 1526)
SGSPHKF,
(SEQ ID NO: 1527)
SGSPHKI,
(SEQ ID NO: 1528)
SGSPHKL,
(SEQ ID NO: 1529)
SGSPHKY,
(SEQ ID NO: 1530)
SGSPHTR,
(SEQ ID NO: 1531)
SHSPHKR,
(SEQ ID NO: 1532)
SGSPHGA,
(SEQ ID NO: 1533)
HDSPHKR,
(SEQ ID NO: 1534)
DDSPHKS,
(SEQ ID NO: 1535)
HESPHKS,
(SEQ ID NO: 1536)
NYSPHKI,
(SEQ ID NO: 1537)
SGSPHSR,
(SEQ ID NO: 1538)
SGSPHSL,
(SEQ ID NO: 1539)
SGSPHSS,
(SEQ ID NO: 1541)
SCSPHRK,
(SEQ ID NO: 1542)
SGSPHFL,
(SEQ ID NO: 1543)
LLSPHWK,
(SEQ ID NO: 1544)
NGSPHSK,
(SEQ ID NO: 1545)
PGSPHSK,
(SEQ ID NO: 1546)
GGSPHSK,
(SEQ ID NO: 1547)
TGSPHSK,
(SEQ ID NO: 1548)
SVSPHGK,
(SEQ ID NO: 1549)
SGSPHTK,
(SEQ ID NO: 1550)
IGSPHSK,
(SEQ ID NO: 1551)
DGSPHSK,
(SEQ ID NO: 1552)
SGSPHNK,
(SEQ ID NO: 1553)
LGSPHSK,
(SEQ ID NO: 1554)
AGSPHSK,
(SEQ ID NO: 1555)
EGSPHSK,
(SEQ ID NO: 1556)
SASPHSK,
(SEQ ID NO: 1557)
SGSPHAK,
(SEQ ID NO: 1558)
HDSPHKI,
(SEQ ID NO: 1559)
YDSPHKS,
(SEQ ID NO: 1560)
HDSPHKT,
(SEQ ID NO: 1561)
RGSPHKR,
(SEQ ID NO: 1562)
HGSPHSK,
(SEQ ID NO: 1563)
RDSPHKS,
(SEQ ID NO: 1564)
NDSPHKS,
(SEQ ID NO: 1565)
QDSPHKI,
(SEQ ID NO: 1566)
PDSPHKI,
(SEQ ID NO: 1567)
PDSPHKS,
(SEQ ID NO: 1568)
MGSPHSK,
(SEQ ID NO: 1569)
HDSPHKH,
(SEQ ID NO: 1570)
QVSPHKS,
(SEQ ID NO: 1571)
HNSPHKS,
(SEQ ID NO: 1572)
NGSPHKR,
(SEQ ID NO: 1573)
HDSPHKY,
(SEQ ID NO: 1574)
NDSPHKI,
(SEQ ID NO: 1575)
HDSPHKL,
(SEQ ID NO: 1576)
HPSPHWK,
(SEQ ID NO: 1577)
HDSPHKM,
or
(SEQ ID NO: 1578)
HSSPHRS;

(ii) an amino acid sequence comprising any portion of an amino acid sequence in (i), e.g., any 2, 3, 4, 5, or 6 amino acids, e.g., consecutive amino acids, thereof;
(iii) an amino acid sequence comprising one, two, or three but no more than four modifications, e.g., substitutions (e.g., conservative substitutions), insertions, or deletions, relative to any of the amino acid sequences in (i); or
(iv) an amino acid sequence comprising one, two, or three but no more than four different amino acids, relative to any one of the amino acid sequences in (i).
19. The AAV particle of any one of embodiments 1-18, wherein [N1]-[N2]-[N3] is or comprises:

(i)
(SEQ ID NO: 1369)
GSGSPHSKA,
(SEQ ID NO: 1370)
GHDSPHKSG,
(SEQ ID NO: 1586)
VSGSPHSKA,
(SEQ ID NO: 1579)
GSGSPHARM,
(SEQ ID NO: 1580)
GSGSPHVKS,
(SEQ ID NO: 1581)
GQDSPHKSG,
(SEQ ID NO: 1582)
GSGSPHASR,
(SEQ ID NO: 1583)
GSGSPHVKI,
(SEQ ID NO: 1584)
GSGSPHKKN,
(SEQ ID NO: 1585)
GSGSPHVRM,
(SEQ ID NO: 1587)
CSGSPHSKA,
(SEQ ID NO: 1588)
GSGSPHRKA,
(SEQ ID NO: 1589)
CSGSPHKTS,
(SEQ ID NO: 1590)
CSHSPHKSG,
(SEQ ID NO: 1591)
GQSSPHRSG,
(SEQ ID NO: 1592)
GRGSPHASR,
(SEQ ID NO: 1593)
GRGSPHSKA,
(SEQ ID NO: 1594)
GSGSPHKFG,
(SEQ ID NO: 1595)
GSGSPHKIG,
(SEQ ID NO: 1596)
GSGSPHKLG,
(SEQ ID NO: 1597)
GSGSPHKTS,
(SEQ ID NO: 1598)
GSGSPHKTT,
(SEQ ID NO: 1599)
GSGSPHKTY,
(SEQ ID NO: 1600)
GSGSPHKYG,
(SEQ ID NO: 1601)
GSGSPHSKD,
(SEQ ID NO: 1602)
GSGSPHSKP,
(SEQ ID NO: 1603)
GSGSPHTRG,
(SEQ ID NO: 1604)
GSGSPHVRG,
(SEQ ID NO: 1605)
GSHSPHKRG,
(SEQ ID NO: 1606)
GSHSPHKSG,
(SEQ ID NO: 1607)
VSGSPHASR,
(SEQ ID NO: 1608)
VSGSPHGAR,
(SEQ ID NO: 1609)
VSGSPHKFG,
(SEQ ID NO: 1610)
GHDSPHKRG,
(SEQ ID NO: 1611)
GDDSPHKSG,
(SEQ ID NO: 1612)
GHESPHKSA,
(SEQ ID NO: 1613)
GHDSPHKSA,
(SEQ ID NO: 1614)
GNYSPHKIG,
(SEQ ID NO: 1615)
GHDSPHKSR,
(SEQ ID NO: 1616)
GSGSPHSKL,
(SEQ ID NO: 1617)
GSGSPHSRA,
(SEQ ID NO: 1618)
GSGSPHSKR,
(SEQ ID NO: 1619)
GSGSPHSLR,
(SEQ ID NO: 1620)
GSGSPHSRG,
(SEQ ID NO: 1621)
GSGSPHSSR,
(SEQ ID NO: 1622)
RVGSPHSKA,
(SEQ ID NO: 1623)
GSCSPHRKA,
(SEQ ID NO: 1624)
GSGSPHFLR,
(SEQ ID NO: 1625)
GSGSPHSKW,
(SEQ ID NO: 1626)
GSGSPHSKS,
(SEQ ID NO: 1627)
GLLSPHWKA,
(SEQ ID NO: 1628)
GSGSPHVRR,
(SEQ ID NO: 1629)
GSGSPHSKV,
(SEQ ID NO: 1630)
MSGSPHSKA,
(SEQ ID NO: 1631)
RNGSPHSKA,
(SEQ ID NO: 1632)
TSGSPHSKA,
(SEQ ID NO: 1633)
ISGSPHSKA,
(SEQ ID NO: 1634)
GPGSPHSKA,
(SEQ ID NO: 1635)
GSGSPHSKT,
(SEQ ID NO: 1636)
ESGSPHSKA,
(SEQ ID NO: 1637)
SSGSPHSKA,
(SEQ ID NO: 1638)
GNGSPHSKA,
(SEQ ID NO: 1639)
ASGSPHSKA,
(SEQ ID NO: 1640)
NSGSPHSKA,
(SEQ ID NO: 1641)
LSGSPHSKA,
(SEQ ID NO: 1642)
GGGSPHSKA,
(SEQ ID NO: 1643)
KSGSPHSKA,
(SEQ ID NO: 1644)
GGGSPHSKS,
(SEQ ID NO: 1645)
GSGSPHSKG,
(SEQ ID NO: 1646)
HSGSPHSKA,
(SEQ ID NO: 1647)
GTGSPHSKA,
(SEQ ID NO: 1648)
PSGSPHSKA,
(SEQ ID NO: 1649)
GSVSPHGKA,
(SEQ ID NO: 1650)
RSGSPHSKA,
(SEQ ID NO: 1651)
GSGSPHTKA,
(SEQ ID NO: 1652)
GIGSPHSKA,
(SEQ ID NO: 1653)
WSGSPHSKA,
(SEQ ID NO: 1654)
DSGSPHSKA,
(SEQ ID NO: 1655)
IDGSPHSKA,
(SEQ ID NO: 1656)
GSGSPHNKA,
(SEQ ID NO: 1657)
GLGSPHSKS,
(SEQ ID NO: 1658)
DAGSPHSKA,
(SEQ ID NO: 1659)
DGGSPHSKA,
(SEQ ID NO: 1660)
MEGSPHSKA,
(SEQ ID NO: 1661)
ENGSPHSKA,
(SEQ ID NO: 1662)
GSASPHSKA,
(SEQ ID NO: 1663)
GNGSPHSKS,
(SEQ ID NO: 1664)
KNGSPHSKA,
(SEQ ID NO: 1665)
KEGSPHSKA,
(SEQ ID NO: 1666)
AIGSPHSKA,
(SEQ ID NO: 1667)
GSGSPHSKN,
(SEQ ID NO: 1668)
GSGSPHAKA,
(SEQ ID NO: 1669)
GHDSPHKIG,
(SEQ ID NO: 1670)
GYDSPHKSG,
(SEQ ID NO: 1671)
GHESPHKSG,
(SEQ ID NO: 1672)
GHDSPHKTG,
(SEQ ID NO: 1673)
GRGSPHKRG,
(SEQ ID NO: 1581)
GQDSPHKSG,
(SEQ ID NO: 1674)
GHDSPHKSL,
(SEQ ID NO: 1675)
GHGSPHSKA,
(SEQ ID NO: 1676)
GHDSPHKSE,
(SEQ ID NO: 1586)
VSGSPHSKA,
(SEQ ID NO: 1677)
GRDSPHKSG,
(SEQ ID NO: 1678)
GNDSPHKSV,
(SEQ ID NO: 1679)
GQDSPHKIG,
(SEQ ID NO: 1680)
GHDSPHKSV,
(SEQ ID NO: 1681)
GPDSPHKIG,
(SEQ ID NO: 1682)
GPDSPHKSG,
(SEQ ID NO: 1683)
GHDSPHKSW,
(SEQ ID NO: 1684)
GHDSPHKSN,
(SEQ ID NO: 1685)
GMGSPHSKT,
(SEQ ID NO: 1686)
GHDSPHKHG,
(SEQ ID NO: 1687)
GQVSPHKSG,
(SEQ ID NO: 1688)
GDDSPHKSV,
(SEQ ID NO: 1689)
GHNSPHKSG,
(SEQ ID NO: 1690)
GNGSPHKRG,
(SEQ ID NO: 1691)
GHDSPHKYG,
(SEQ ID NO: 1692)
GHDSPHKSQ,
(SEQ ID NO: 1693)
GNDSPHKIG,
(SEQ ID NO: 1694)
GHDSPHKSK,
(SEQ ID NO: 1695)
GHDSPHKLW,
(SEQ ID NO: 1696)
GHPSPHWKG,
(SEQ ID NO: 1697)
GHDSPHKMG,
(SEQ ID NO: 1698)
GHDSPHKMA,
or
(SEQ ID NO: 1699)
GHSSPHRSG;

(ii) an amino acid sequence comprising any portion of an amino acid sequence in (i), e.g., any 2, 3, 4, 5, 6, 7, or 8 amino acids, e.g., consecutive amino acids, thereof;
(iii) an amino acid sequence comprising one, two, or three but no more than four modifications, e.g., substitutions (e.g., conservative substitutions), insertions, or deletions, relative to any of the amino acid sequences in (i); or
(iv) an amino acid sequence comprising one, two, or three but no more than four different amino acids, relative to any one of the amino acid sequences in (i).
20. The AAV particle of any one of embodiments 1-19, wherein [N3] comprises SK, KA, KS, or SG.
21. The AAV particle of any one of embodiments 1-20, wherein [N3] is or comprises SKA, KSG, or KYG.
22. The AAV particle of any one of embodiments 1-21, wherein [N2]-[N3] comprises SPHSK (SEQ ID NO: 1372), SPHKS (SEQ ID NO: 1375), or SPHKY (SEQ ID NO: 1386).
23. The AAV particle of any one of embodiments 1-22, wherein [N2]-[N3] is or comprises SPHSKA (SEQ ID NO: 941).
24. The AAV particle of any one of embodiments 1-22, wherein [N2]-[N3] is or comprises SPHKSG (SEQ ID NO: 946).
25. The AAV particle of any one of embodiments 1-22, wherein [N2]-[N3] is or comprises SPHKYG (SEQ ID NO: 966) 26. The AAV particle of any one of embodiments 1-25, wherein [N1] comprises GS, SG, GH, or HD.
27. The AAV particle of any one of embodiments 1-26, wherein [N1] is or comprises GSG.
28. The AAV particle of any one of embodiments 1-26, wherein [N1] is or comprises GHD.
29. The AAV particle of any one of embodiments 1-23 or 26-27, wherein [N1]-[N2]-[N3] comprises SGSPHSK (SEQ ID NO: 1512).
30. The AAV particle of any one of embodiments 1-22, 24, 26, or 28, wherein [N1]-[N2]-[N3] comprises HDSPHKS (SEQ ID NO: 1513).
31. The AAV particle of any one of embodiments 1-22 or 25-27, wherein [N1]-[N2]-[N3] comprises SGSPHKYG (SEQ ID NO: 1700).
32. The AAV particle of any one of embodiments 1-8, 10, 12-23, 26-27, or 29, wherein [N1]-[N2]-[N3] is or comprises GSGSPHSKA (SEQ ID NO: 1369).
33. The AAV particle of any one of embodiments 1-9, 11-22, 24, 26, 28, or 30, wherein [N1]-[N2]-[N3] is or comprises GHDSPHKSG (SEQ ID NO: 1370).
34. The AAV particle of any one of embodiments 1-8, 10, 12-22, 25-27, or 31, wherein [N1]-[N2]-[N3] is or comprises GSGSPHKYG (SEQ ID NO: 1600).
35. The AAV particle of any one of embodiments 1-34, wherein [N1]-[N2]-[N3] replaces positions 453-455, numbered according to the amino acid sequence of SEQ ID NO: 138.
36. The AAV particle of any one of embodiments 1-35, wherein the AAV capsid variant comprises an amino acid other than Q at position 456 (e.g., W, K, R, G, L, V, S, P, H, K, I, M, A, E, or F), an amino acid other than N at position 457 (e.g., Y, C, K, T, H, R, D, V, S, P, G, W, E, F, A, I, M, Q, or L), an amino acid other than Q at position 458 (e.g., G, K, H, R, T, L, D, A, P, I, F, V, M, W, Y, S, E, N, or Y), and/or an amino acid other than Q at position 459 (e.g., H, L, R, W, K, A, P, E, M, I, S, G, N, Y, C, V, T, D, or V), relative to a reference sequence numbered according to SEQ ID NO: 138.
37. The AAV particle of any one of embodiments 1-36, wherein the AAV capsid variant comprises an amino acid other than Q at position 462 (e.g., W, K, R, G, L, V, S, P, H, K, I, M, A, E, or F), an amino acid other than N at position 463 (e.g., Y, C, K, T, H, R, D, V, S, P, G, W, E, F, A, I, M, Q, or L), an amino acid other than Q at position 464 (e.g., G, K, H, R, T, L, D, A, P, I, F, V, M, W, Y, S, E, N, or Y), and/or an amino acid other than Q at position 465 (e.g., H, L, R, W, K, A, P, E, M, I, S, G, N, Y, C, V, T, D, or V), relative to a reference sequence numbered according to SEQ ID NO: 981, 982, 36, 37, 39, 40, 42-46, 48, 49, 50, 52, 53, 56, or 57.
38. The AAV particle of any one of embodiments 1-37, wherein the AAV capsid variant comprises:
   (a) the amino acid Q at position 456, the amino acid N at position 457, the amino acid Q at position 458, and/or the amino acid Q at position 459, relative to a reference sequence numbered according to SEQ ID NO: 138; or
   (b) the amino acid Q at position 462, the amino acid N at position 463, the amino acid Q at position 464, and/or the amino acid Q at position 465, relative to a reference sequence numbered according to SEQ ID NO: 981, 982, 36, 37, 39, 40, 42-46, 48, 49, 50, 52, 53, 56, or 57.
39. The AAV particle of any one of embodiments 1-38, wherein the AAV capsid variant further comprises [N4], wherein [N4] comprises X7 X8 X9 X10, and wherein:
   (a) position X7 is: Q, W, K, R, G, L, V, S, P, H, K, I, M, A, E, or F;
   (b) position X8 is: N, Y, C, K, T, H, R, D, V, S, P, G, W, E, F, A, I, M, Q, or L;
   (c) position X9 is: Q, G, K, H, R, T, L, D, A, P, I, F, V, M, W, Y, S, E, N, or Y; and
   (d) position X10 is: Q, H, L, R, W, K, A, P, E, M, I, S, G, N, Y, C, V, T, D, or V;
   optionally wherein the AAV capsid variant comprises an amino acid modification, e.g., a conservative substitution, of any of the aforesaid amino acids in (a)-(d).
40. The AAV particle of embodiment 39, wherein:
   (a) position X7 of [N4] is Q or R;
   (b) position X8 of [N4] is N or R;
   (c) position X9 of [N4] is Q or R; and
   (d) position X10 of [N4] is Q, L, or R.
41. The AAV particle of embodiment 39 or 40, wherein [N4] is or comprises:

(i)
(SEQ ID NO: 1701)
QNQQ,
(SEQ ID NO: 1702)
WNQQ,
(SEQ ID NO: 1703)
QYYV,
(SEQ ID NO: 1704)
RRQQ,
(SEQ ID NO: 1705)
GCGQ,
(SEQ ID NO: 1706)
LRQQ,
(SEQ ID NO: 1707)
RNQQ,
(SEQ ID NO: 1708)
VNQQ,
(SEQ ID NO: 1709)
FRLQ,
(SEQ ID NO: 1710)
FNQQ,
(SEQ ID NO: 1711)
LLQQ,
(SEQ ID NO: 1712)
SNQQ,
(SEQ ID NO: 1713)
RLQQ,
(SEQ ID NO: 1714)
LNQQ,
(SEQ ID NO: 1715)
QRKL,
(SEQ ID NO: 1716)
LRRQ,
(SEQ ID NO: 1717)
QRLR,
(SEQ ID NO: 1718)
QRRL,
(SEQ ID NO: 1719)
RRLQ,
(SEQ ID NO: 1720)
RLRQ,
(SEQ ID NO: 1721)
SKRQ,
(SEQ ID NO: 1722)
QLYR,
(SEQ ID NO: 1723)
QLTV,
(SEQ ID NO: 1724)
QNKQ,
(SEQ ID NO: 1725)
KNQQ,
(SEQ ID NO: 1726)
QKQQ,
(SEQ ID NO: 1727)
QTQQ,
(SEQ ID NO: 1728)
QNHQ,
(SEQ ID NO: 1729)
QHQQ,
(SEQ ID NO: 1730)
QNQH,
(SEQ ID NO: 1731)
QHRQ,
(SEQ ID NO: 1732)
LTQQ,
(SEQ ID NO: 1733)
QNQW,
(SEQ ID NO: 1734)
QNTH,
(SEQ ID NO: 1735)
RRRQ,
(SEQ ID NO: 1736)
QYQQ,
(SEQ ID NO: 1737)
QNDQ,
(SEQ ID NO: 1738)
QNRH,
(SEQ ID NO: 1739)
RDQQ,
(SEQ ID NO: 1740)
PNLQ,
(SEQ ID NO: 1741)
HVRQ,
(SEQ ID NO: 1742)
PNQH,
(SEQ ID NO: 1743)
HNQQ,
(SEQ ID NO: 1744)
QSQQ,
(SEQ ID NO: 1745)
QPAK,
(SEQ ID NO: 1746)
QNLA,
(SEQ ID NO: 1747)
QNQL,
(SEQ ID NO: 1748)
QGQQ,
(SEQ ID NO: 1749)
LNRQ,
(SEQ ID NO: 1750)
QNPP,
(SEQ ID NO: 1751)
QNLQ,
(SEQ ID NO: 1752)
QDQE,
(SEQ ID NO: 1753)
QDQQ,
(SEQ ID NO: 1755)
HWQQ,
(SEQ ID NO: 1756)
PNQQ,
(SEQ ID NO: 1757)
PEQQ,
(SEQ ID NO: 1758)
QRTM,
(SEQ ID NO: 1759)
LHQH,
(SEQ ID NO: 1760)
QHRI,
(SEQ ID NO: 1761)
QYIH,
(SEQ ID NO: 1762)
QKFE,
(SEQ ID NO: 1763)
QFPS,
(SEQ ID NO: 1764)
QNPL,
(SEQ ID NO: 1765)
QAIK,
(SEQ ID NO: 1766)
QNRQ,
(SEQ ID NO: 1767)
QYQH,
(SEQ ID NO: 1768)
QNPQ,
(SEQ ID NO: 1769)
QHQL,
(SEQ ID NO: 1770)
QSPP,
(SEQ ID NO: 1771)
QAKL,
(SEQ ID NO: 1772)
KSQQ,
(SEQ ID NO: 1773)
QDRP,
(SEQ ID NO: 1774)
QNLG,
(SEQ ID NO: 1775)
QAFH,
(SEQ ID NO: 1776)
QNAQ,
(SEQ ID NO: 1777)
HNQL,
(SEQ ID NO: 1778)
QKLN,
(SEQ ID NO: 1779)
QNVQ,
(SEQ ID NO: 1780)
QAQQ,
(SEQ ID NO: 1781)
QTPP,
(SEQ ID NO: 1782)
QPPA,
(SEQ ID NO: 1783)
QERP,
(SEQ ID NO: 1784)
QDLQ,
(SEQ ID NO: 1785)
QAMH,
(SEQ ID NO: 1786)
QHPS,
(SEQ ID NO: 1787)
PGLQ,
(SEQ ID NO: 1788)
QGIR,
(SEQ ID NO: 1789)
QAPA,
(SEQ ID NO: 1790)
QIPP,
(SEQ ID NO: 1791)
QTQL,
(SEQ ID NO: 1792)
QAPS,
(SEQ ID NO: 1793)
QNTY,
(SEQ ID NO: 1794)
QDKQ,
(SEQ ID NO: 1795)
QNHL,
(SEQ ID NO: 1796)
QIGM,
(SEQ ID NO: 1797)
LNKQ,
(SEQ ID NO: 1798)
PNQL,
(SEQ ID NO: 1799)
QLQQ,
(SEQ ID NO: 2897)
QRMS,
(SEQ ID NO: 2907)
QGIL,
(SEQ ID NO: 2917)
QDRQ,
(SEQ ID NO: 3081)
RDWQ,
(SEQ ID NO: 3238)
QERS,
(SEQ ID NO: 3590)
QNYQ,
(SEQ ID NO: 3849)
QRTC,
(SEQ ID NO: 3850)
QIGH,
(SEQ ID NO: 3851)
QGAI,
(SEQ ID NO: 3852)
QVPP,
(SEQ ID NO: 3853)
QVQQ,
(SEQ ID NO: 3854)
LMRQ,
(SEQ ID NO: 3855)
QYSV,
(SEQ ID NO: 3856)
QAIT,
(SEQ ID NO: 3857)
QKTL,
(SEQ ID NO: 3858)
QLHH,
(SEQ ID NO: 3859)
QNII,
(SEQ ID NO: 3860)
QGHH,
(SEQ ID NO: 3861)
QSKV,
(SEQ ID NO: 3862)
QLPS,
(SEQ ID NO: 3863)
IGKQ,
(SEQ ID NO: 3864)
QAIH,
(SEQ ID NO: 3865)
QHGL,
(SEQ ID NO: 3866)
QFMC,
(SEQ ID NO: 3867)
QNQM,
(SEQ ID NO: 3868)
QHLQ,
(SEQ ID NO: 3869)
QPAR,
(SEQ ID NO: 3870)
QSLQ,
(SEQ ID NO: 3871)
QSQL,
(SEQ ID NO: 3872)
HSQQ,
(SEQ ID NO: 3873)
QMPS,
(SEQ ID NO: 3874)
QGSL,
(SEQ ID NO: 3875)
QVPA,
(SEQ ID NO: 3876)
HYQQ,
(SEQ ID NO: 3877)
QVPS,
(SEQ ID NO: 3878)
RGEQ,
(SEQ ID NO: 3879)
PGQQ,
(SEQ ID NO: 3880)
LEQQ,
(SEQ ID NO: 3881)
QNQS,
(SEQ ID NO: 3882)
QKVI,
(SEQ ID NO: 3883)
QNND,
(SEQ ID NO: 3884)
QSVH,
(SEQ ID NO: 3885)
QPLG,
(SEQ ID NO: 3886)
HNQE,
(SEQ ID NO: 3887)
QIQQ,
(SEQ ID NO: 3888)
QVRN,
(SEQ ID NO: 3889)
PSNQ,
(SEQ ID NO: 3890)
QVGH,
(SEQ ID NO: 3891)
QRDI,
(SEQ ID NO: 3892)
QMPN,
(SEQ ID NO: 3893)
RGLQ,
(SEQ ID NO: 3894)
PSLQ,
(SEQ ID NO: 3895)
QRDQ,
(SEQ ID NO: 3896)
QAKG,
(SEQ ID NO: 3897)
QSAH,
(SEQ ID NO: 3898)
QSTM,
(SEQ ID NO: 3899)
QREM,
(SEQ ID NO: 3900)
QYRA,
(SEQ ID NO: 3901)
QRQQ,
(SEQ ID NO: 3902)
QWQQ,
(SEQ ID NO: 3903)
QRMN,
(SEQ ID NO: 3904)
GDSQ,
(SEQ ID NO: 3905)
QKIS,
(SEQ ID NO: 3906)
PSMQ,
(SEQ ID NO: 3907)
SPRQ,
(SEQ ID NO: 3908)
MEQQ,
(SEQ ID NO: 3909)
QYQN,
(SEQ ID NO: 3910)
QIRQ,
(SEQ ID NO: 3911)
QSVQ,
(SEQ ID NO: 3912)
RSQQ,
(SEQ ID NO: 3913)
QNKL,
(SEQ ID NO: 3914)
QIQH,
(SEQ ID NO: 3915)
PRQQ,
(SEQ ID NO: 3916)
HTQQ,
(SEQ ID NO: 3917)
QRQH,
(SEQ ID NO: 3918)
RNQE,
(SEQ ID NO: 3919)
QSKQ,
(SEQ ID NO: 3920)
QNQP,
(SEQ ID NO: 3921)
QSPQ,
(SEQ ID NO: 3922)
QTRQ,
(SEQ ID NO: 3923)
QNLH,
(SEQ ID NO: 3924)
QNQE,
(SEQ ID NO: 3925)
LNQP,
(SEQ ID NO: 3926)
QNQD,
(SEQ ID NO: 3927)
QNLL,
(SEQ ID NO: 3928)
QLVI,
(SEQ ID NO: 3929)
RTQE,
(SEQ ID NO: 3930)
QTHQ,
(SEQ ID NO: 3931)
QDQH,
(SEQ ID NO: 3932)
QSQH,
(SEQ ID NO: 3933)
VRQQ,
(SEQ ID NO: 3934)
AWQQ,
(SEQ ID NO: 3935)
QSVP,
(SEQ ID NO: 3936)
QNIQ,
(SEQ ID NO: 3937)
LDQQ,
(SEQ ID NO: 3938)
PDQQ,
(SEQ ID NO: 3939)
ESQQ,
(SEQ ID NO: 3940)
QRQL,
(SEQ ID NO: 3941)
QIIV,
(SEQ ID NO: 3942)
QKQS,
(SEQ ID NO: 3943)
QSHQ,
(SEQ ID NO: 3944)
QFVV,
(SEQ ID NO: 3945)
QSQP,
(SEQ ID NO: 3946)
QNEQ,
(SEQ ID NO: 3947)
INQQ,
(SEQ ID NO: 3948)
RNRQ,
(SEQ ID NO: 3949)
RDQK,
(SEQ ID NO: 3950)
QWKR,
(SEQ ID NO: 3951)
ENRQ,
(SEQ ID NO: 3952)
QTQP,
(SEQ ID NO: 3953)
QKQL,
(SEQ ID NO: 3954)
RNQL,
(SEQ ID NO: 3955)
ISIQ,
(SEQ ID NO: 3956)
QTVC,
(SEQ ID NO: 3957)
QQIM,
(SEQ ID NO: 3958)
LNHQ,
(SEQ ID NO: 3959)
QNQA,
(SEQ ID NO: 3960)
QMIH,
(SEQ ID NO: 3961)
RNHQ,
or
(SEQ ID NO: 3962)
QKMN;

(ii) an amino acid sequence comprising any portion of an amino acid sequence in (i), e.g., any 2, or 3 amino acids, e.g., consecutive amino acids, thereof;
(iii) an amino acid sequence comprising one, two, or three but no more than four modifications, e.g., substitutions (e.g., conservative substitutions), insertions, or deletions, relative to any of the amino acid sequences in (i); or
(iv) an amino acid sequence comprising one, two, or three but no more than four different amino acids, relative to any one of the amino acid sequences in (i).
42. The AAV particle of any one of embodiments 39-41, wherein [N1]-[N2]-[N3]-[N4] is or comprises: (i) the amino acid sequence of any of SEQ ID NOs: 1800-2241;
   (ii) an amino acid sequence comprising any portion of an amino acid sequence in (i), e.g., any 2, 3, 4, 5, 6, 7, 8, 9, 10, 11, or 12 amino acids, e.g., consecutive amino acids, thereof;
   (iii) an amino acid sequence comprising one, two, or three but no more than four modifications, e.g., substitutions (e.g., conservative substitutions), insertions, or deletions, relative to any of the amino acid sequences in (i); or
   (iv) an amino acid sequence comprising one, two, or three but no more than four different amino acids, relative to any one of the amino acid sequences in (i).
43. The AAV particle of any one of embodiments 37-42, wherein [N1]-[N2]-[N3]-[N4] is or comprises GSGSPHSKAQNQQ (SEQ ID NO: 1801).
44. The AAV particle of any one of embodiments 37-42, wherein [N1]-[N2]-[N3]-[N4] is or comprises GHDSPHKSGQNQQ (SEQ ID NO: 1800).
45. The AAV particle of any one of embodiments 37-42, wherein [N1]-[N2]-[N3]-[N4] is or comprises GSGSPHKYGQNQQT (SEQ ID NO: 910).
46. The AAV particle of any one of embodiments 1-45, wherein the AAV capsid variant comprises an amino acid other than T at position 450 (e.g., S, Y, M, A, C, I, R, L, D, F, V, Q, N, H, E, or G), an amino acid other than I at position 451 (e.g., M, P, E, N, D, S, A, T, G, Q, F, V, L, C, H, R, W, or L), and/or an amino acid other than N at position 452 (e.g., M, E, G, Y, W, T, I, Q, F, V, A, L, I, P, K, R, H, S, D, or S), relative to a reference sequence numbered according to any one of SEQ ID NOs: 69, 70, 71, 4, 5, 6, 7, 8, 36-59, 138, 981 or 982.
47. The AAV particle of any one of embodiments 1-46, wherein the AAV capsid variant comprises:
   (i) the amino acid T at position 450, the amino acid I at position 451, and/or the amino acid N at position 452, relative to a reference sequence numbered according to any one of SEQ ID NOs: 138, 981, or 982;
   (ii) the amino acid T at position 450, the amino acid E at position 451, and/or the amino acid R at position 452, numbered according to SEQ ID NO: 36; or
   (iii) the amino acid T at position 450, the amino acid E at position 451, and/or the amino acid N at position 452, numbered according to SEQ ID NO: 69.
48. The AAV particle of any one of embodiments 1-47, wherein the AAV capsid variant further comprises [N0], wherein [N0] comprises X_{A} XB and X_{C}, and wherein:
   (a) position X_{A} is: T, S, Y, M, A, C, I, R, L, D, F, V, Q, N, H, E, or G;
   (b) position XB is: I, M, P, E, N, D, S, A, T, G, Q, F, V, L, C, H, R, W, or L; and
   (c) position X_{C} is: N, M, E, G, Y, W, T, I, Q, F, V, A, L, I, P, K, R, H, S, D, or S; and optionally wherein the AAV capsid variant comprises an amino acid modification, e.g., a conservative substitution, of any of the aforesaid amino acids in (a)-(c).
49. The AAV particle of embodiment 48, wherein [N0] is or comprises TIN, TEN, TER, SMN, TIM, YLS, GLS, MPE, MEG, MEY, AEW, CEW, ANN, IPE, ADM, IEY, ADY, IET, MEW, CEY, RIN, MEI, LEY, ADW, IEI, DIM, FEQ, MEF, CDQ, LPE, IEN, MES, AEI, VEY, IIN, TSN, IEV, MEM, AEV, MDA, VEW, AEQ, LEW, MEL, MET, MEA, IES, MEV, CEI, ATN, MDG, QEV, ADQ, NMN, IEM, ISN, TGN, QQQ, HDW, IEG, TII, TFP, TEK, EIN, TVN, TFN, SIN, TSY, ELH, AlN, SVN, TDN, TFH, TVH, TSS, TID, TCN, NIN, TEH, AEM, AIK, TDK, TFK, SDQ, TEI, NTN, TET, SIK, TEL, TEA, TAN, TIY, TFS, TES, TTN, TED, TNN, EVH, TIS, TVR, TDR, TIK, NHI, TIP, ESD, TDL, TVP, TVI, AEH, NCL, TVK, NAD, TIT, NCV, TIR, NAL, VIN, TIQ, TEF, TRE, QGE, SEK, NVN, GGE, EFV, SDK, TEQ, EVQ, TEY, NCW, TDV, SDI, NSI, NSL, EVV, TEP, SEL, TWQ, TEV, AVN, GVL, TLN, TEG, TRD, NAI, AEN, AET, ETA, NNL, or any dipeptide thereof.
50. The AAV particle of embodiment 48 or 49, wherein [N0]-[N1]-[N2]-[N3]-[N4] is or comprises:
   (i) the amino acid sequence of any one of SEQ ID NOs: 2242-2886;
   (ii) an amino acid sequence comprising any portion of an amino acid sequence in (i), e.g., any 2, 3, 4, 5, 6, 7, 8, 9, 10, 11, 12, 13, 14, or 15 amino acids, e.g., consecutive amino acids, thereof;
   (iii) an amino acid sequence comprising one, two, or three but no more than four modifications, e.g., substitutions (e.g., conservative substitutions), insertions, or deletions, relative to any of the amino acid sequences in (i); or
   (iv) an amino acid sequence comprising one, two, or three but no more than four different amino acids, relative to any one of the amino acid sequences in (i).
51. The AAV particle of any one of embodiments 48-50, wherein [N0]-[N1]-[N2]-[N3]-[N4] is or comprises TINGSGSPHSKAQNQQ (SEQ ID NO: 2242).
52. The AAV particle of any one of embodiments 48-50, wherein [N0]-[N1]-[N2]-[N3]-[N4] is or comprises TINGHDSPHKSGQNQQ (SEQ ID NO: 2243).
53. The AAV particle of any one of embodiments 48-50, wherein [N0]-[N1]-[N2]-[N3]-[N4] is or comprises TINGSGSPHKYGQNQQT (SEQ ID NO: 5246).
54. The AAV particle of any one of embodiments 1-53, wherein [N1]-[N2]-[N3] is present in loop IV of the AAV capsid variant.
55. The AAV particle of any one of embodiments 48-54, wherein [N0] and [N4] are present in loop IV of the AAV capsid variant.
56. The AAV particle of any one of embodiments 48-55, wherein [N0] is present immediately subsequent to position 449, relative to a reference sequence numbered according to the amino acid sequence of SEQ ID NO: 138.
57. The AAV particle of any one of embodiments 48-55, wherein [N0] is present immediately subsequent to position 449, relative to a reference sequence numbered according to the amino acid sequence of any one of SEQ ID NO: 69, 70, 71, 4, 5, 6, 7, 8, 36-59, 981, or 982.
58. The AAV particle of any one of embodiments 48-57, wherein [N0] replaces positions 450, 451, and 452 (e.g., T450, 1451, and N452), relative to a reference sequence numbered according to SEQ ID NO: 138.
59. The AAV particle of any one of embodiments 48-58, wherein [N0] replaces positions 450-452 (e.g., T450, 1451, and N452), relative to a reference sequence numbered according to any one of SEQ ID NOs: 69, 70, 71, 4, 5, 6, 7, 8, 36-59, 981, or 982.
60. The AAV particle of any one of embodiments 48-59, wherein [N0] corresponds to positions 450-452 of any one of SEQ ID NOs: 69, 70, 71, 4, 5, 6, 7, 8, 36-59, 138, 981 or 982.
61. The AAV particle of any one of embodiments 48-60, wherein [N0] is present immediately subsequent to position 449 and wherein [N0] replaces positions 450-452 (e.g., T450, 1451, and N452), relative to a reference sequence numbered according to SEQ ID NO: 138.
62. The AAV particle of any one of embodiments 48-61, wherein [N0] is present immediately subsequent to position 449 and wherein [N0] replaces positions 450-452 (e.g., T450, I451, and N452), relative to a reference sequence numbered according to any one of SEQ ID NOs: 69, 70, 71, 4, 5, 6, 7, 8, 36-59, 981 or 982.
63. The AAV particle of any one of embodiments 1-62, wherein [N1] is present immediately subsequent to position 452, relative to a reference sequence numbered according to the amino acid sequence of SEQ ID NO: 138.
64. The AAV particle of any one of embodiments 1-63, wherein [N1] is present immediately subsequent to position 452, relative to a reference sequence numbered according to the amino acid sequence of SEQ ID NO: 981 or 982.
65. The AAV particle of any one of embodiments 1-64, wherein [N1] replaces positions 453-455 (e.g., G453, S454, and G455), relative to a reference sequence numbered according to SEQ ID NO: 138.
66. The AAV particle of any one of embodiments 1-65, wherein [N1] replaces positions 453 (e.g., G453), relative to a reference sequence numbered according to SEQ ID NO: 138.
67. The AAV particle of any one of embodiments 1-66, wherein [N1] replaces positions 453-455 (e.g., G453, S454, and G455), relative to a reference sequence numbered according to SEQ ID NO: 981.
68. The AAV particle of any one of embodiments 1-66 or 67, wherein [N1] replaces positions 453-455, relative to a reference sequence numbered according to SEQ ID NO: 982.
69. The AAV particle of any one of embodiments 1-68, wherein [N1] is present immediately subsequent to position 452 and wherein [N1] replaces positions 453-455 (e.g., G453, S454, and G455), relative to a reference sequence numbered according to SEQ ID NO: 138.
70. The AAV particle of any one of embodiments 1-64, or 66, wherein [N1] is present immediately subsequent to position 452 and wherein [N1] replaces positions 453 (e.g., G453), relative to a reference sequence numbered according to SEQ ID NO: 138.
71. The AAV particle of any one of embodiments 1-64, 66, or 70, wherein [N1] is present immediately subsequent to position 452 and wherein [N1] replaces positions 453-455, relative to a reference sequence numbered according to SEQ ID NO: 981 or 982.
72. The AAV particle of any one of embodiments 1-71, wherein [N1] corresponds to positions 453-455 of any one of SEQ ID NOs: 69, 70, 71, 4, 5, 6, 7, 8, 36-59, 981 or 982.
73. The AAV particle of any one of embodiment 1-72, wherein the AAV capsid variant comprises an amino acid other than S at position 454 and/or an amino acid other than G at position 455, numbered according to SEQ ID NO: 138, 981, or 982.
74. The AAV particle of any one of embodiments 1-73, wherein the AAV capsid variant comprises the amino acid H at position 454 and the amino acid D at position 455, numbered according to SEQ ID NO: 138.
75. The AAV particle of any one of embodiments 1-74, wherein the AAV capsid variant comprises a substitution at position 454 (e.g., S454H) and/or a substitution at position 455 (e.g., G455D), numbered according to SEQ ID NO: 138 or 982.
76. The AAV particle of any one of embodiments 1-75, wherein the AAV capsid variant comprises the amino acid H at position 454 and the amino acid D at position 455, and further comprises the amino acid sequence SPHSKA (SEQ ID NO: 941) immediately subsequent to position 455, relative to a reference sequence numbered according to SEQ ID NO: 138.
77. The AAV particle of any one of embodiments 1-71, wherein the AAV capsid variant comprises the amino acid H at position 454 and the amino acid D at position 455, relative to a reference sequence numbered according to SEQ ID NO: 982.
78. The AAV particle of any one of embodiments 1-77, wherein the AAV capsid variant comprises the amino acid H at position 454 and the amino acid D at position 455, and further comprises the amino acid sequence SPHSKA (SEQ ID NO: 941) immediately subsequent to position 455, relative to a reference sequence numbered according to SEQ ID NO: 982.
79. The AAV particle of any one of embodiments 1-71, wherein the AAV capsid variant comprises the amino acid S at position 454 and the amino acid G at position 455, relative to a reference sequence numbered according to SEQ ID NO: 138.
80. The AAV particle of any one of embodiments 1-71 or 79, wherein the AAV capsid variant comprises the amino acid S at position 454 and the amino acid G at position 455, and further comprises the amino acid sequence SPHSKA (SEQ ID NO: 941) immediately subsequent to position 455, relative to a reference sequence numbered according to SEQ ID NO: 138.
81. The AAV particle of any one of embodiments 1-71, 79, or 80, wherein the AAV capsid variant comprises the amino acid S at position 454 and the amino acid G at position 455, relative to a reference sequence numbered according to SEQ ID NO: 981.
82. The AAV particle of any one of embodiments 1-71 or 79-81, wherein the AAV capsid variant comprises the amino acid S at position 454 and the amino acid G at position 455, and further comprises the amino acid sequence SPHSKA (SEQ ID NO: 941) immediately subsequent to position 455, relative to a reference sequence numbered according to SEQ ID NO: 981.
83. The AAV particle of any one of embodiments 1-82, wherein [N2] is present immediately subsequent to position 455, relative to a reference sequence numbered according to the amino acid sequence of SEQ ID NO: 138.
84. The AAV particle of any one of embodiments 1-82, wherein [N2] corresponds to positions 456-458 (e.g., S456, P457, H458) of SEQ ID NO: 981 or 982.
85. The AAV particle of any one of embodiments 1-84, wherein [N2] corresponds to positions 456-458 (e.g., S456, P457, H458) of any one of SEQ ID NOs: 69, 70, 71, 4, 5, 6, 7, 8, or 36-59.
86. The AAV particle of any one of embodiments 1-85, wherein [N2]-[N3] is present immediately subsequent to position 455, relative to a reference sequence numbered according to the amino acid sequence of SEQ ID NO: 138.
87. The AAV particle of any one of embodiments 1-86, wherein [N2] is present immediately subsequent to position 455, relative to a reference sequence numbered according to the amino acid sequence of SEQ ID NO: 981 or 982.
88. The AAV particle of any one of embodiments 1-87, wherein [N2]-[N3] is present immediately subsequent to position 455, relative to a reference sequence numbered according to the amino acid sequence of SEQ ID NO: 981 or 982.
89. The AAV particle of any one of embodiments 1-88, wherein [N2]-[N3] corresponds to positions 456-461 (e.g., S456, P457, H458, S459, K460, A461) of SEQ ID NO: 981.
90. The AAV particle of any one of embodiments 1-88, wherein [N2]-[N3] corresponds to positions 456-461 (e.g., S456, P457, H458, K459, S460, G461) of SEQ ID NO: 982.
91. The AAV particle of any one of embodiments 1-88, wherein [N2] is present immediately subsequent to [N1].
92. The AAV particle of any one of embodiments 1-64, 66, 70, or 71, wherein [N3] is present immediately subsequent to [N2] and replaces positions 454 and 455 (e.g., S454 and G455), numbered according to SEQ ID NO: 138.
93. The AAV particle of any one of embodiments 1-64, 66, 70, 71, or 92, wherein [N3] is present immediately subsequent to [N1]-[N2] and replaces positions 454 and 455 (e.g., S454 and G455), numbered according to SEQ ID NO: 138.
94. The AAV particle of any one of embodiments 39-93, wherein [N4] is present immediately subsequent to position 455, relative to a reference sequence numbered according to the amino acid sequence of SEQ ID NO: 138.
95. The AAV particle of any one of embodiments 39-94, wherein [N4] replaces positions 456-459 (e.g., Q456, N457, Q458, and Q459), relative to a reference sequence numbered according to the amino acid sequence of SEQ ID NO: 138.
96. The AAV particle of any one of embodiments 39-95, wherein [N4] corresponds to positions 462-465 (e.g., Q462, N463, Q464, Q465) of SEQ ID NO: 981 or 982.
97. The AAV particle of any one of embodiments 39-96, wherein [N2]-[N3]-[N4] replaces positions 456-459 (e.g., Q456, N457, Q458, and Q459), relative to a reference sequence numbered according to the amino acid sequence of SEQ ID NO: 138.
98. The AAV particle of any one of embodiments 39-97, wherein [N2]-[N3]-[N4] is present immediately subsequent to position 455, and wherein [N2]-[N3]-[N4] replaces positions 456-459 (e.g., Q456, N457, Q458, and Q459), relative to a reference sequence numbered according to the amino acid sequence of SEQ ID NO: 138.
99. The AAV particle of any one of embodiments 39-98, wherein [N2]-[N3]-[N4] corresponds to positions 456-465 (e.g., S456, P457, H458, S459, K460, A461, Q462, N463, Q464, Q465) of SEQ ID NO: 981.
100. The AAV particle of any one of embodiments 39-99, wherein [N2]-[N3]-[N4] corresponds to positions 456-465 (e.g., S456, P457, H458, K459, S460, G461, Q462, N463, Q464, Q465) of SEQ ID NO: 982.
101. The AAV particle of any one of embodiments 39-100, wherein [N2]-[N3]-[N4] corresponds to positions 456-465 of any one of SEQ ID NOs: 69, 70, 71, 4, 5, 6, 7, 8, or 36-59.
102. The AAV particle of any one of embodiments 39-101, wherein [N1]-[N2]-[N3]-[N4] replaces positions 453-459 (e.g., G453, S454, G455, Q456, N457, Q458, and Q459), relative to a reference sequence numbered according to the amino acid sequence of SEQ ID NO: 138.
103. The AAV particle of any one of embodiments 39-102, wherein [N1]-[N2]-[N3]-[N4] is present immediately subsequent to position 452, and wherein [N1]-[N2]-[N3]-[N4] replaces positions 453-459 (e.g., G453, S454, G455, Q456, N457, Q458, and Q459), relative to a reference sequence numbered according to the amino acid sequence of SEQ ID NO: 138.
104. The AAV particle of any one of embodiments 39-99, 102, or 103, wherein [N1]-[N2]-[N3]-[N4] corresponds to positions 453-465 (e.g., G453, S454, G455, S456, P457, H458, S459, K460, A461, Q462, N463, Q464, Q465) of SEQ ID NO: 981.
105. The AAV particle of any one of embodiments 39-98 or 100-103, wherein [N1]-[N2]-[N3]-[N4] corresponds to positions 453-465 (e.g., G453, H454, D455, S456, P457, H458, K459, S460, G461, Q462, N463, Q464, Q465) of SEQ ID NO: 982.
106. The AAV particle of any one of embodiments 39-98, or 101-103, wherein [N1]-[N2]-[N3]-[N4] corresponds to positions 453-465 of any one of SEQ ID NOs: 69, 70, 71, 4, 5, 6, 7, 8, or 36-59.
107. The AAV particle of any one of embodiments 1-98 or 102-104 wherein [N1]-[N2]-[N3] corresponds to positions 453-461 (e.g., G453, S454, G455, S456, P457, H458, S459, K460, A461) of SEQ ID NO: 981.
108. The AAV particle of any one of embodiments 1-98, 100, 102, 103, or 105, wherein [N1]-[N2]-[N3] corresponds to positions 453-461 (e.g., G453, H454, D455, S456, P457, H458, K459, S460, G461) of SEQ ID NO: 982.
109. The AAV particle of any one of embodiments 40-98, 101-103, or 106, wherein [N1]-[N2]-[N3] corresponds to positions 453-461 of any one of SEQ ID NOs: 69, 70, 71, 4, 5, 6, 7, 8, or 36-59.
110. The AAV particle of any one of embodiments 39-109, wherein [N0]-[N1]-[N2]-[N3]-[N4] replaces positions 450-459 (e.g., T450, 1451, N452, G453, S454, G455, Q456, N457, Q458, and Q459), relative to a reference sequence numbered according to the amino acid sequence of SEQ ID NO: 138.
111. The AAV particle of any one of embodiments 39-110, wherein [N0]-[N1]-[N2]-[N3]-[N4] is present immediately subsequent to position 449, and wherein [N0]-[N1]-[N2]-[N3]-[N4] replaces positions 450-459 (e.g., T450, 1451, N452, G453, S454, G455, Q456, N457, Q458, and Q459), relative to a reference sequence numbered according to the amino acid sequence of SEQ ID NO: 138.
112. The AAV particle of any one of embodiments 39-99, 102-104, 110 or 111, wherein [N0]-[N1]-[N2]-[N3]-[N4] corresponds to positions 450-465 (e.g., T450, I451, N452, G453, S454, G455, S456, P457, H458, S459, K460, A461, Q462, N463, Q464, Q465) of SEQ ID NO: 981.
113. The AAV particle of any one of embodiments 39-98, 100-103, or 105-111, wherein [N0]-[N1]-[N2]-[N3]-[N4] corresponds to positions 450-465 (e.g., T450, 1451, N452, G453, H454, D455, S456, P457, H458, K459, S460, G461, Q462, N463, Q464, Q465) of SEQ ID NO: 982.
114. The AAV particle of any one of embodiments 48-98, 101-103, 106, or 109, wherein [N0]-[N1]-[N2]-[N3]-[N4] corresponds to positions 450-465 of any one of SEQ ID NOs: 69, 70, 71, 4, 5, 6, 7, 8, or 36-59.
115. The AAV particle of any one of embodiments 39-113, wherein [N4] replaces positions 462-465 (e.g., Q462, N463, Q464, and Q465), relative to a reference sequence numbered according to the amino acid sequence of SEQ ID NO: 981 or 982.
116. The AAV particle of any one of embodiments 39-115, wherein [N2]-[N3]-[N4] replaces positions 462-465 (e.g., Q462, N463, Q464, and Q465), relative to a reference sequence numbered according to the amino acid sequence of SEQ ID NO: 981 or 982.
117. The AAV particle of any one of embodiments 39-116, wherein [N2]-[N3]-[N4] is present immediately subsequent to position 455, and wherein [N2]-[N3]-[N4] replaces positions 462-465 (e.g., Q462, N463, Q464, and Q465), relative to a reference sequence numbered according to the amino acid sequence of SEQ ID NO: 981 or 982.
118. The AAV particle of any one of embodiments 1-117, wherein [N3] is present immediately subsequent to [N2].
119. The AAV particle of any one of embodiments 1-118, wherein the AAV capsid variant comprises from N-terminus to C-terminus, [N2]-[N3].
120. The AAV particle of any one of embodiments 1-119, wherein the AAV capsid variant comprises from N-terminus to C-terminus, [N1]-[N2]-[N3].
121. The AAV particle of any one of embodiments 28-120, wherein the AAV capsid variant comprises from N-terminus to C-terminus, [N0]-[N1]-[N2]-[N3].
122. The AAV particle of any one of embodiments 25-121, wherein the AAV capsid variant comprises from N-terminus to C-terminus, [N1]-[N2]-[N3]-[N4].
123. The AAV particle of any one of embodiments 25-122, wherein the AAV capsid variant comprises from N-terminus to C-terminus, [N0]-[N1]-[N2]-[N3]-[N4].
124. The AAV particle of any one of the preceding embodiments, wherein the AAV capsid variant comprises an amino acid other T at position 460 (e.g., N, I, C, H, R, L, D, Y, A, M, Q, I, E, K, P, G or S), numbered according to the amino acid sequence of SEQ ID NO: 138.
125. The AAV particle of any one of the preceding embodiments, wherein the AAV capsid variant comprises the amino acid N, I, C, H, R, L, D, Y, A, M, Q, I, E, K, P, G or S at position 460, numbered according to the amino acid sequence of SEQ ID NO: 138.
126. The AAV particle of any one of the preceding embodiments, wherein the AAV capsid variant comprises an amino acid other T at position 466 (e.g., N, I, C, H, R, L, D, Y, A, M, Q, I, E, K, P, G or S), numbered according to the amino acid sequence of any one of SEQ ID NOs: 69, 70, 71, 4, 5, 6, 7, 8, 36-59, 981 or 982.
127. The AAV particle of any one of the preceding embodiments, wherein the AAV capsid variant comprises the amino acid N, I, C, H, R, L, D, Y, A, M, Q, I, E, K, P, G or S at position 466, numbered according to the amino acid sequence of any one of SEQ ID NOs: 69, 70, 71, 4, 5, 6, 7, 8, 36-59, 981 or 982.
128. The AAV particle of any one of the preceding embodiments, wherein the AAV capsid variant comprises an amino acid other K at position 449 (e.g., an E, an N, or a T), numbered according to the amino acid sequence of any one of SEQ ID NOs: 69, 70, 71, 4, 5, 6, 7, 8, 36-59, 138, 981, or 982.
129. The AAV particle of any one of the preceding embodiments, wherein the AAV capsid variant comprises the amino E, N, or T at position 449, numbered according to the amino acid sequence of any one of SEQ ID NOs: 69, 70, 71, 4, 5, 6, 7, 8, 36-59, 138, 981 or 982.
130. An AAV particle comprising an AAV capsid variant and a nucleic acid encoding a SOD1 targeting polynucleotide (e.g., a SOD1 targeting RNA agent, e.g., a siRNA), wherein the AAV capsid variant comprises an amino acid sequence having the following formula: [N1]-[N2]-[N3] (SEQ ID NO: 3963), wherein:
   (i) [N1] comprises positions X1, X2, and X3, wherein position X2 is S and position X3 is G;
   (ii) [N2] comprises the amino acid sequence SPH; and
   (iii) [N3] comprises positions X4, X5, and X6, wherein position X5 is K.
131. The AAV particle of embodiment 130, wherein:
   (i) X4 of [N3] is S, T, N, or A; and
   (ii) X5 of [N3] is A, V, T, S, G, R, L, or N;
   optionally wherein the AAV capsid variant comprises an amino acid modification, e.g., a conservative substitution, of any of the aforesaid amino acids in (i) or (ii).
132. The AAV particle of embodiment 130 or 131, wherein X4 is S and/or X5 is A.
133. The AAV particle of any one of embodiments 130-132, wherein [N3] comprises SK, TK, NK, AK, KA, KV, KT, KS, KG, KR, KL, or KN.
134. The AAV particle of any one of embodiments 130-133, wherein [N3] is or comprises SKA, SKV, SKT, SKS, SKG, SKR, TKA, NKA, SKL, SKN, or AKA.
135. The AAV particle of any one of embodiments 130-134, wherein [N3] is or comprises SKA.
136. The AAV particle of any one of embodiments 130-135, wherein [N2]-[N3] comprises SPHSK (SEQ ID NO: 1372), SPHTK (SEQ ID NO: 1396), SPHNK (SEQ ID NO: 1397), or SPHAK (SEQ ID NO: 1398).
137. The AAV particle of any one of embodiments 130-136, wherein [N2]-[N3] is or comprises:

(i)
(SEQ ID NO: 941)
SPHSKA,
(SEQ ID NO: 1409)
SPHSKV,
(SEQ ID NO: 1403)
SPHSKT,
(SEQ ID NO: 962)
SPHSKS,
(SEQ ID NO: 1404)
SPHSKG,
(SEQ ID NO: 978)
SPHSKR,
(SEQ ID NO: 1411)
SPHTKA,
(SEQ ID NO: 1406)
SPHNKA,
(SEQ ID NO: 960)
SPHSKL,
(SEQ ID NO: 1407)
SPHSKN,
or
(SEQ ID NO: 1408)
SPHAKA;

(ii) an amino acid sequence comprising any portion of an amino acid sequence in (i), e.g., any 2, 3, 4, or 5 amino acids, e.g., consecutive amino acids, thereof;
(iii) an amino acid sequence comprising one, two, or three but no more than four modifications, e.g., substitutions (e.g., conservative substitutions), insertions, or deletions, relative to any of the amino acid sequences in (i); or
(iv) an amino acid sequence comprising one, two, or three but no more than four different amino acids, relative to any one of the amino acid sequences in (i).
138. The AAV particle of any one of embodiments 130-137, wherein [N2]-[N3] is or comprises SPHSKA (SEQ ID NO: 941).
139. The AAV particle of any one of embodiments 130-138, which comprises an amino acid other than G at position 453 (e.g., M, T, I, E, S, A, N, V, L, K, H, P, R, W, or D), numbered according to SEQ ID NO: 138 or 981.
140. The AAV particle of any one of embodiments 130-139, which comprises the amino acid G at position 453, numbered according to SEQ ID NO: 138 or 981.
141. The AAV particle of any one of embodiments 130-140, wherein X1 of [N1] is chosen from: G, M, T, I, E, S, A, N, V, L, K, H, P, R, W, or D; optionally wherein the AAV capsid variant comprises an amino acid modification, e.g., a conservative substitution, of any of the aforesaid amino acids.
142. The AAV particle of any one of embodiments 130-141, wherein [N1] comprises SG, GS, MS, TS, IS, ES, SS, AS, NS, VS, LS, KS, HS, PS, RS, WS, or DS.
143. The AAV particle of any one of embodiments 130-142, wherein [N1] is or comprises: GSG, MSG, TSG, ISG, ESG, SSG, ASG, NSG, VSG, LSG, KSG, HSG, PSG, RSG, WSG, or DSG.
144. The AAV particle of any one of embodiments 130-143, wherein [N1] is or comprises GSG or VSG.
145. The AAV particle of any one of embodiments 130-144, wherein [N1]-[N2] comprises SGSPH (SEQ ID NO: 1424).
146. The AAV particle of any one of embodiments 130-145, wherein [N1]-[N2] is or comprises:

(i)
(SEQ ID NO: 1456)
GSGSPH,
(SEQ ID NO: 1459)
VSGSPH,
(SEQ ID NO: 1471)
MSGSPH,
(SEQ ID NO: 1473)
TSGSPH,
(SEQ ID NO: 1474)
ISGSPH,
(SEQ ID NO: 1476)
ESGSPH,
(SEQ ID NO: 1477)
SSGSPH,
(SEQ ID NO: 1479)
ASGSPH,
(SEQ ID NO: 1480)
NSGSPH,
(SEQ ID NO: 1481)
LSGSPH,
(SEQ ID NO: 1483)
KSGSPH,
(SEQ ID NO: 1484)
HSGSPH,
(SEQ ID NO: 1486)
PSGSPH,
(SEQ ID NO: 1488)
RSGSPH,
(SEQ ID NO: 1490)
WSGSPH,
(SEQ ID NO: 1491)
DSGSPH;

(ii) an amino acid sequence comprising any portion of an amino acid sequence in (i), e.g., any 2, 3, 4, or 5 amino acids, e.g., consecutive amino acids, thereof;
(iii) an amino acid sequence comprising one, two, or three but no more than four modifications, e.g., substitutions (e.g., conservative substitutions), insertions, or deletions, relative to any of the amino acid sequences in (i); or
(iv) an amino acid sequence comprising one, two, or three but no more than four different amino acids, relative to any one of the amino acid sequences in (i).
147. The AAV particle of any one of embodiments 130-146, wherein [N1]-[N2]-[N3] is or comprises:

(i)
(SEQ ID NO: 1369)
GSGSPHSKA,
(SEQ ID NO: 1586)
VSGSPHSKA,
(SEQ ID NO: 1629)
GSGSPHSKV,
(SEQ ID NO: 1630)
MSGSPHSKA,
(SEQ ID NO: 1632)
TSGSPHSKA,
(SEQ ID NO: 1633)
ISGSPHSKA,
(SEQ ID NO: 1635)
GSGSPHSKT,
(SEQ ID NO: 1636)
ESGSPHSKA,
(SEQ ID NO: 1637)
SSGSPHSKA,
(SEQ ID NO: 1626)
GSGSPHSKS,
(SEQ ID NO: 1639)
ASGSPHSKA,
(SEQ ID NO: 1640)
NSGSPHSKA,
(SEQ ID NO: 1641)
LSGSPHSKA,
(SEQ ID NO: 1643)
KSGSPHSKA,
(SEQ ID NO: 1645)
GSGSPHSKG,
(SEQ ID NO: 1618)
GSGSPHSKR,
(SEQ ID NO: 1646)
HSGSPHSKA,
(SEQ ID NO: 1648)
PSGSPHSKA,
(SEQ ID NO: 1650)
RSGSPHSKA,
(SEQ ID NO: 1651)
GSGSPHTKA,
(SEQ ID NO: 1653)
WSGSPHSKA,
(SEQ ID NO: 1654)
DSGSPHSKA,
(SEQ ID NO: 1656)
GSGSPHNKA,
(SEQ ID NO: 1616)
GSGSPHSKL,
(SEQ ID NO: 1667)
GSGSPHSKN,
or
(SEQ ID NO: 1668)
GSGSPHAKA;

(ii) an amino acid sequence comprising any portion of an amino acid sequence in (i), e.g., any 2, 3, 4, 5, 6, 7, 8, or 9 amino acids, e.g., consecutive amino acids, thereof;
(iii) an amino acid sequence comprising one, two, or three but no more than four modifications, e.g., substitutions (e.g., conservative substitutions), insertions, or deletions, relative to any of the amino acid sequences in (i); or
(iv) an amino acid sequence comprising one, two, or three but no more than four different amino acids, relative to any one of the amino acid sequences in (i).
148. The AAV particle of any one of embodiments 130-147, wherein [N1]-[N2]-[N3] is or comprises GSGSPHSKA (SEQ ID NO: 1369) or VSGSPHSKA (SEQ ID NO: 1586).
149. The AAV particle of any one of embodiments 130-148, wherein the AAV capsid variant comprises an amino acid other than Q at position 456 (e.g., R, P, H, L, K, I, G, S, M, or E), an amino acid other than N at position 457 (e.g., D, V, S, P, T, G, Y, W, E, R, H, K, F, A, I, L, or M), an amino acid other than Q at position 458 (e.g., R, L, A, P, H, T, I, F, K, V, M, G, W, Y, S, E, N, or D), an amino acid other than Q at position 459 (e.g., H, K, A, L, P, E, M, I, S, N, R, Y, C, V, T, W, D, G), and/or an amino acid other than T at position 460 (e.g., I, N, S, H, R, L, D, Y, A, or Q), relative to a reference sequence numbered according to SEQ ID NO: 138.
150. The AAV particle of any one of embodiments 130-149, wherein the AAV capsid variant comprises an amino acid other than Q at position 462 (e.g., R, P, H, L, K, I, G, S, M, or E), an amino acid other than N at position 463 (e.g., D, V, S, P, T, G, Y, W, E, R, H, K, F, A, I, L, or M), an amino acid other than Q at position 464 (e.g., R, L, A, P, H, T, I, F, K, V, M, G, W, Y, S, E, N, or D), an amino acid other than Q at position 465 (e.g., H, K, A, L, P, E, M, I, S, N, R, Y, C, V, T, W, D, G), and/or an amino acid other than T at position 466 (e.g., I, N, S, H, R, L, D, Y, A, or Q), relative to a reference sequence numbered according to SEQ ID NO: 981.
151. The AAV particle of any one of embodiments 130-150, wherein the AAV capsid variant comprises the amino acid Q at position 456, the amino acid N at position 457, the amino acid Q at position 458, the amino acid Q at position 459, and/or the amino acid T at position 460, relative to a reference sequence numbered according to SEQ ID NO: 138.
152. The AAV particle of any one of embodiments 130-151, wherein the AAV capsid variant comprises the amino acid Q at position 462, the amino acid N at position 463, the amino acid Q at position 464, the amino acid Q at position 465, and/or the amino acid T at position 466, numbered according to SEQ ID NO: 981 153. The AAV particle of any one of embodiments 130-152, wherein the AAV capsid variant further comprises [N4] wherein [N4] comprises X7, X8, X9, X10, and X11, wherein:
   (a) X7 is Q, R, P, H, L, K, I, G, S, M, or E;
   (b) X8 is N, D, V, S, P, T, G, Y, W, E, R, H, K, F, A, I, L, or M;
   (c) X9 is Q, R, L, A, P, H, T, I, F, K, V, M, G, W, Y, S, E, N, D;
   (d) X10 is Q, H, K, A, L, P, E, M, I, S, N, R, Y, C, V, T, W, D, G; and
   (e) X11 is T, I, N, S, H, R, L, D, Y, A, Q;
   optionally wherein the AAV capsid variant comprises an amino acid modification, e.g., a conservative substitution, of any of the aforesaid amino acids in (a)-(e).
154. The AAV particle of embodiment 153, wherein [N4] is or comprises:

(i)
(SEQ ID NO: 3964)
QNQQT,
(SEQ ID NO: 3965)
QNRHT,
(SEQ ID NO: 3966)
RDQQT,
(SEQ ID NO: 3967)
PNLQT,
(SEQ ID NO: 3968)
HVRQT,
(SEQ ID NO: 3969)
PNQHT,
(SEQ ID NO: 3970)
QSQQT,
(SEQ ID NO: 3971)
QNQQI,
(SEQ ID NO: 3972)
QPAKT,
(SEQ ID NO: 3973)
QTQQN,
(SEQ ID NO: 3974)
QNLAT,
(SEQ ID NO: 3975)
QNQLT,
(SEQ ID NO: 3976)
QGQQT,
(SEQ ID NO: 3977)
LNRQS,
(SEQ ID NO: 3978)
HNQQT,
(SEQ ID NO: 3979)
QNPPT,
(SEQ ID NO: 3980)
QNLQT,
(SEQ ID NO: 3981)
QYQQT,
(SEQ ID NO: 3982)
QDQET,
(SEQ ID NO: 3983)
QNHQT,
(SEQ ID NO: 3984)
QDQQT,
(SEQ ID NO: 3985)
HWQQT,
(SEQ ID NO: 3986)
PNQQT,
(SEQ ID NO: 3987)
QNQLI,
(SEQ ID NO: 3988)
PEQQT,
(SEQ ID NO: 3989)
QRTMT,
(SEQ ID NO: 3990)
QNQQH,
(SEQ ID NO: 3991)
LHQHT,
(SEQ ID NO: 3992)
QHRIT,
(SEQ ID NO: 3993)
QYIHT,
(SEQ ID NO: 3994)
QKFET,
(SEQ ID NO: 3995)
QFPST,
(SEQ ID NO: 3996)
HNQQR,
(SEQ ID NO: 3997)
QAIKT,
(SEQ ID NO: 3998)
QNRQT,
(SEQ ID NO: 3999)
QYQHT,
(SEQ ID NO: 4002)
QNPQS,
(SEQ ID NO: 4005)
QHQLT,
(SEQ ID NO: 4006)
QSPPT,
(SEQ ID NO: 4007)
QAKLT,
(SEQ ID NO: 4010)
KSQQT,
(SEQ ID NO: 4049)
QDRPT,
(SEQ ID NO: 4050)
QSQQL,
(SEQ ID NO: 4051)
QAFHT,
(SEQ ID NO: 4052)
QKQQD,
(SEQ ID NO: 4053)
QNAQT,
(SEQ ID NO: 4054)
HNQLT,
(SEQ ID NO: 4055)
QNQQY,
(SEQ ID NO: 4056)
QKLNT,
(SEQ ID NO: 4057)
QNVQT,
(SEQ ID NO: 4058)
QAQQT,
(SEQ ID NO: 4059)
QNLQA,
(SEQ ID NO: 4060)
QTPPT,
(SEQ ID NO: 4061)
QYQHA,
(SEQ ID NO: 4063)
QGQQA,
(SEQ ID NO: 4064)
QPPAT,
(SEQ ID NO: 4065)
QERPT,
(SEQ ID NO: 4066)
QDLQT,
(SEQ ID NO: 4067)
QAMHT,
(SEQ ID NO: 4068)
LNQQT,
(SEQ ID NO: 4069)
QHPST,
(SEQ ID NO: 4070)
PGLQT,
(SEQ ID NO: 4071)
QGIRT,
(SEQ ID NO: 4072)
QAPAT,
(SEQ ID NO: 4073)
QSQQI,
(SEQ ID NO: 4074)
QIPPT,
(SEQ ID NO: 4075)
QTQLT,
(SEQ ID NO: 4076)
QAPST,
(SEQ ID NO: 4077)
QNTYA,
(SEQ ID NO: 4078)
QNQHI,
(SEQ ID NO: 4079)
QNHLT,
(SEQ ID NO: 4080)
QIGMT,
(SEQ ID NO: 4082)
LNKQT,
(SEQ ID NO: 4083)
QLQQT,
(SEQ ID NO: 4084)
QRMST,
(SEQ ID NO: 4085)
QGILT,
(SEQ ID NO: 4086)
QDRQT,
(SEQ ID NO: 4087)
RDWQT,
(SEQ ID NO: 4088)
QNTHD,
(SEQ ID NO: 4089)
PNLQI,
(SEQ ID NO: 4090)
QERST,
(SEQ ID NO: 4091)
QNYQT,
(SEQ ID NO: 4092)
QRTCT,
(SEQ ID NO: 4093)
QIGHT,
(SEQ ID NO: 4094)
QGAIT,
(SEQ ID NO: 4095)
QVPPT,
(SEQ ID NO: 4096)
QVQQI,
(SEQ ID NO: 4097)
LMRQT,
(SEQ ID NO: 4487)
QYSVT,
(SEQ ID NO: 4488)
QAITT,
(SEQ ID NO: 4489)
QKTLT,
(SEQ ID NO: 4490)
QNQWT,
(SEQ ID NO: 4491)
QLHHT,
(SEQ ID NO: 4492)
QNIII,
(SEQ ID NO: 4493)
QGHHT,
(SEQ ID NO: 4494)
QSKVT,
(SEQ ID NO: 4495)
QLPST,
(SEQ ID NO: 4496)
IGKQT,
(SEQ ID NO: 4497)
QAIHT,
(SEQ ID NO: 4498)
QHGLT,
(SEQ ID NO: 4499)
QFMCT,
(SEQ ID NO: 4500)
QHLQT,
(SEQ ID NO: 4501)
QNHQN,
(SEQ ID NO: 4502)
QPART,
(SEQ ID NO: 4503)
QSLQT,
(SEQ ID NO: 4504)
QSQLT,
(SEQ ID NO: 4505)
QDRQS,
(SEQ ID NO: 4506)
QMPST,
(SEQ ID NO: 4507)
QGSLT,
(SEQ ID NO: 4508)
QVPAT,
(SEQ ID NO: 4509)
QDKQT,
(SEQ ID NO: 4510)
HYQQT,
(SEQ ID NO: 4511)
QVPST,
(SEQ ID NO: 4512)
RGEQT,
(SEQ ID NO: 4513)
PGQQT,
(SEQ ID NO: 4514)
QSLQI,
(SEQ ID NO: 4515)
LEQQT,
(SEQ ID NO: 4516)
QNQST,
(SEQ ID NO: 4517)
QKVIT,
(SEQ ID NO: 4518)
QNNDQ,
(SEQ ID NO: 4519)
QSVHT,
(SEQ ID NO: 4520)
QPLGT,
(SEQ ID NO: 4521)
HNQET,
(SEQ ID NO: 4522)
QNLQI,
(SEQ ID NO: 4523)
QIQQT,
(SEQ ID NO: 4524)
QVRNT,
(SEQ ID NO: 4525)
PSNQT,
(SEQ ID NO: 4526)
QVGHT,
(SEQ ID NO: 4527)
QRDIT,
(SEQ ID NO: 4528)
QMPNT,
(SEQ ID NO: 4529)
RGLQT,
(SEQ ID NO: 4530)
QKQQT,
(SEQ ID NO: 4531)
PSLQT,
(SEQ ID NO: 4532)
QRDQT,
(SEQ ID NO: 4533)
QAKGT,
(SEQ ID NO: 4534)
QSAHT,
(SEQ ID NO: 4535)
QSTMT,
(SEQ ID NO: 4536)
QREMT,
(SEQ ID NO: 4537)
QYRAT,
(SEQ ID NO: 4538)
QWQQT,
(SEQ ID NO: 4539)
QRMNT,
(SEQ ID NO: 4540)
GDSQT,
(SEQ ID NO: 4541)
QKIST,
(SEQ ID NO: 4542)
PSMQT,
(SEQ ID NO: 4543)
SPRQT,
(SEQ ID NO: 4544)
MEQQT,
(SEQ ID NO: 4545)
QYQNT,
(SEQ ID NO: 4546)
QHQQT,
(SEQ ID NO: 4547)
INQQT,
(SEQ ID NO: 4548)
PNQQH,
(SEQ ID NO: 4549)
ENRQT,
(SEQ ID NO: 4550)
QTQQA,
or
(SEQ ID NO: 4551)
QNQAT;

(ii) an amino acid sequence comprising any portion of an amino acid sequence in (i), e.g., any 2, 3, or 4 amino acids, e.g., consecutive amino acids, thereof;
(iii) an amino acid sequence comprising one, two, or three but no more than four modifications, e.g., substitutions (e.g., conservative substitutions), insertions, or deletions, relative to any of the amino acid sequences in (i); or
(iv) an amino acid sequence comprising one, two, or three but no more than four different amino acids, relative to any one of the amino acid sequences in (i).
155. The AAV particle of embodiment 153 or 154, wherein [N1]-[N2]-[N3]-[N4] is or comprises: (i) the amino acid sequence of any of SEQ ID NOs: 200 or 2887-3076;
   (ii) an amino acid sequence comprising any portion of an amino acid sequence in (i), e.g., any 2, 3, 4, 5, 6, 7, 8, 9, 10, 11, 12, or 13 amino acids, e.g., consecutive amino acids, thereof;
   (iii) an amino acid sequence comprising one, two, or three but no more than four modifications, e.g., substitutions (e.g., conservative substitutions), insertions, or deletions, relative to any of the amino acid sequences in (i); or
   (iv) an amino acid sequence comprising one, two, or three but no more than four different amino acids, relative to any one of the amino acid sequences in (i).
156. The AAV particle of any one of embodiments 153-155, wherein [N1]-[N2]-[N3]-[N4] is or comprises GSGSPHSKAQNQQT (SEQ ID NO: 200).
157. The AAV particle of any one of embodiments 153-155, wherein [N1]-[N2]-[N3]-[N4] is or comprises VSGSPHSKAQNQQT (SEQ ID NO: 903).
158. The AAV particle of any one of embodiments 130-157, wherein the AAV capsid variant comprises an amino acid other than K at position 449 (e.g., T, E, or N), T at position 450 (e.g., S, E, A, N, V, Q, or G), an amino acid other than I at position 451 (e.g., F, E, V, L, D, S, C, T, A, N, H, R, G, or W), and/or an amino acid other than N at position 452 (e.g., I, P, K, R, H, S, M, Q, D, T, L, A, Y, V, F, E, W, or G), relative to a reference sequence numbered according to SEQ ID NO: 138.
159. The AAV particle of any one of embodiments 130-157, wherein the AAV capsid variant comprises:
   (i) the amino acid K at position 449, the amino acid T at position 450, the amino acid I at position 451, and/or the amino acid N at position 452, relative to a reference sequence numbered according to SEQ ID NO: 138 or 981;
   (ii) the amino acid K at position 449, the amino acid T at position 450, the amino acid E at position 451, and/or the amino acid N at position 452, relative to a reference sequence numbered according to SEQ ID NO: 69; or
   (iii) the amino acid K at position 449, the amino acid T at position 450, the amino acid E at position 451, and/or the amino acid R at position 452, relative to a reference sequence numbered according to SEQ ID NO: 36.
160. The AAV particle of any one of embodiments 130-159, wherein the AAV capsid variant further comprises [N0], wherein [N0] comprises X_{A}, X_{B}, X_{C}, and X_{D}, wherein:
   (a) X_{A} is K, T, E, or N;
   (b) X_{b} is T, S, E, A, N, V, Q, or G;
   (c) X_{C} is I, F, E, V, L, D, S, C, T, A, N, H, R, G, or W; and
   (d) X_{D} is N, I, P, K, R, H, S, M, Q, D, T, L, A, Y, V, F, E, W, or G;
   optionally wherein the AAV capsid variant comprises an amino acid modification, e.g., a conservative substitution, of any of the aforesaid amino acids in (a)-(d).
161. The AAV particle of embodiment 160, wherein [N0] is or comprises:

(i)
(SEQ ID NO: 4557)
KTIN,
(SEQ ID NO: 4568)
KTEN,
(SEQ ID NO: 4560)
KTER,
(SEQ ID NO: 4552)
KTII,
(SEQ ID NO: 4553)
KTFP,
(SEQ ID NO: 4554)
KTEK,
(SEQ ID NO: 4555)
KTVN,
(SEQ ID NO: 4556)
KTFN,
(SEQ ID NO: 4558)
TTIN,
(SEQ ID NO: 4559)
KSIN,
(SEQ ID NO: 4561)
KELH,
(SEQ ID NO: 4562)
KAIN,
(SEQ ID NO: 4563)
KTDN,
(SEQ ID NO: 4564)
KTFH,
(SEQ ID NO: 4565)
KTSN,
(SEQ ID NO: 4566)
ETIN,
(SEQ ID NO: 4567)
NTIN,
(SEQ ID NO: 4569)
KTSS,
(SEQ ID NO: 4570)
KTCN,
(SEQ ID NO: 4571)
KTEH,
(SEQ ID NO: 4572)
KAEM,
(SEQ ID NO: 4573)
KATN,
(SEQ ID NO: 4574)
KAIK,
(SEQ ID NO: 4575)
KTDK,
(SEQ ID NO: 4576)
KTFK,
(SEQ ID NO: 4577)
KSDQ,
(SEQ ID NO: 4578)
KTEI,
(SEQ ID NO: 4579)
KTID,
(SEQ ID NO: 4580)
KNTN,
(SEQ ID NO: 4581)
KTET,
(SEQ ID NO: 4582)
KTEL,
(SEQ ID NO: 4583)
KNIN,
(SEQ ID NO: 4584)
KTEA,
(SEQ ID NO: 4585)
KTAN,
(SEQ ID NO: 4586)
NTIY,
(SEQ ID NO: 4587)
KTFS,
(SEQ ID NO: 4588)
KTES,
(SEQ ID NO: 4589)
KTTN,
(SEQ ID NO: 4590)
KTED,
(SEQ ID NO: 4591)
KTNN,
(SEQ ID NO: 4592)
KEVH,
(SEQ ID NO: 4593)
KTIS,
(SEQ ID NO: 4594)
KTVR,
(SEQ ID NO: 4595)
KTDR,
(SEQ ID NO: 4596)
ETIK,
(SEQ ID NO: 4597)
KNHI,
(SEQ ID NO: 4598)
KESD,
(SEQ ID NO: 4599)
KTIK,
(SEQ ID NO: 4600)
KTDL,
(SEQ ID NO: 4601)
KTVP,
(SEQ ID NO: 4602)
KTVI,
(SEQ ID NO: 4603)
KAEH,
(SEQ ID NO: 4604)
KNCL,
(SEQ ID NO: 4605)
KTVK,
(SEQ ID NO: 4606)
KNAD,
(SEQ ID NO: 4607)
KTIT,
(SEQ ID NO: 4608)
KNCV,
(SEQ ID NO: 4609)
KNAL,
(SEQ ID NO: 4610)
KVIN,
(SEQ ID NO: 4611)
KTEF,
(SEQ ID NO: 4612)
KTRE,
(SEQ ID NO: 4613)
KQGE,
(SEQ ID NO: 4614)
KSEK,
(SEQ ID NO: 4615)
KNVN,
(SEQ ID NO: 4616)
KGGE,
(SEQ ID NO: 4617)
KEFV,
(SEQ ID NO: 4618)
KSDK,
(SEQ ID NO: 4619)
KTEQ,
(SEQ ID NO: 4620)
KEVQ,
(SEQ ID NO: 4621)
KTEY,
(SEQ ID NO: 4622)
KNCW,
(SEQ ID NO: 4623)
KTDV,
(SEQ ID NO: 4624)
KSDI,
(SEQ ID NO: 4625)
KNSI,
(SEQ ID NO: 4626)
KNSL,
(SEQ ID NO: 4627)
KEVV,
(SEQ ID NO: 4628)
KTEP,
(SEQ ID NO: 4629)
KSEL,
(SEQ ID NO: 4630)
KTWQ,
(SEQ ID NO: 4631)
KTEV,
(SEQ ID NO: 4632)
KAVN,
(SEQ ID NO: 4633)
KGVL,
(SEQ ID NO: 4634)
KTEG,
(SEQ ID NO: 4635)
KTRD,
(SEQ ID NO: 4636)
KTGN,
(SEQ ID NO: 4637)
KNAI,
(SEQ ID NO: 4638)
KAEN,
(SEQ ID NO: 4639)
KAET,
(SEQ ID NO: 4640)
KTVH,
(SEQ ID NO: 4641)
KETA,
(SEQ ID NO: 4642)
KNNL,
(SEQ ID NO: 4643)
EAIN,
(SEQ ID NO: 4644)
KSLN,
(SEQ ID NO: 4645)
KTIP,
or
(SEQ ID NO: 4646)
KTIH;

(ii) an amino acid sequence comprising any portion of an amino acid sequence in (i), e.g., any 2, or 3 amino acids, e.g., consecutive amino acids, thereof;
(iii) an amino acid sequence comprising one, two, or three but no more than four modifications, e.g., substitutions (e.g., conservative substitutions), insertions, or deletions, relative to any of the amino acid sequences in (i); or
(iv) an amino acid sequence comprising one, two, or three but no more than four different amino acids, relative to any one of the amino acid sequences in (i).
162. The AAV particle of embodiment 160 or 161, wherein [N0]-[N1]-[N2]-[N3]-[N4] is or comprises:
   (i) the amino acid sequence of any one of SEQ ID NOs: 3239-3526 or 3591-3605;
   (ii) an amino acid sequence comprising any portion of an amino acid sequence in (i), e.g., any 2, 3, 4, 5, 6, 7, 8, 9, 10, 11, 12, 13, 14, 15, 16, or 17 amino acids, e.g., consecutive amino acids, thereof;
   (iii) an amino acid sequence comprising one, two, or three but no more than four modifications, e.g., substitutions (e.g., conservative substitutions), insertions, or deletions, relative to any of the amino acid sequences in (i); or
   (iv) an amino acid sequence comprising one, two, or three but no more than four different amino acids, relative to any one of the amino acid sequences in (i).
163. The AAV particle of any one of embodiments 160-162, wherein [N0]-[N1]-[N2]-[N3]-[N4] is or comprises KTINGSGSPHSKAQNQQT (SEQ ID NO: 5660).
164. The AAV particle of any one of embodiments 160-162, wherein [N0]-[N1]-[N2]-[N3]-[N4] is or comprises KTERVSGSPHSKAQNQQT (SEQ ID NO: 3589) or KTENVSGSPHSKAQNQQT (SEQ ID NO: 3272) . . .
165. An AAV particle comprising an AAV capsid variant and a nucleic acid encoding a SOD1 targeting polynucleotide (e.g., a SOD1 targeting RNA agent, e.g., a siRNA), wherein the AAV capsid variant comprises an amino acid sequence having the following formula: [N1]-[N2]-[N3] (SEQ ID NO: 4647),
wherein:
   (i) [N1] comprises positions X1, X2, and X3, wherein position X2 is an amino acid other than S and position X3 is an amino acid other than G;
   (ii) [N2] comprises the amino acid sequence SPH; and
   (iii) [N3] comprises positions X4, X5, and X6, wherein position X4 is K.
166. The AAV particle of embodiment 165, wherein:
   (i) X5 of [N3] is S, I, T, R, H, Y, L, or M; and
   (ii) X6 of [N3] is G, A, L, E, V, R, W, N, Q, or K;
   optionally wherein the AAV capsid variant comprises an amino acid modification, e.g., a conservative substitution, of any of the aforesaid amino acids in (i) or (ii).
167. The AAV particle of embodiment 165 or 166, wherein X5 is S and/or X6 is G.
168. The AAV particle of any one of embodiments 165-167, wherein [N3] comprises KS, KI, KT, KR, KH, KY, KL, KM, SG, IG, TG, RG, SA, SL, SE, SV, SR, SW, SN, HG, YG, SQ, IV, SK, LW, MG, or MA.
169. The AAV particle of any one of embodiments 165-168, wherein [N3] is or comprises KSG, KIG, KTG, KRG, KSA, KSL, KSE, KSV, KSR, KSW, KSN, KHG, KYG, KSQ, KIV, KSK, KLW, KMG, or KMA.
170. The AAV particle of any one of embodiments 165-169, wherein [N3] is or comprises KSG.
171. The AAV particle of any one of embodiments 165-170, wherein [N2]-[N3] comprises SPHKS (SEQ ID NO: 1375), SPHKI (SEQ ID NO: 1384), SPHKT (SEQ ID NO: 1382), SPHKR (SEQ ID NO: 1388), NPHKS (SEQ ID NO: 4648), SPHKH (SEQ ID NO: 1399), SPHKY (SEQ ID NO: 1386), SPHKL (SEQ ID NO: 1385), or SPHKM (SEQ ID NO: 1400).
172. The AAV particle of any one of embodiments 165-171, wherein [N2]-[N3] is or comprises:

(i)
(SEQ ID NO: 946)
SPHKSG,
(SEQ ID NO: 958)
SPHKIG,
(SEQ ID NO: 1410)
SPHKTG,
(SEQ ID NO: 974)
SPHKRG,
(SEQ ID NO: 4649)
NPHKSG,
(SEQ ID NO: 977)
SPHKSA,
(SEQ ID NO: 1412)
SPHKSL,
(SEQ ID NO: 1413)
SPHKSE,
(SEQ ID NO: 1414)
SPHKSV,
(SEQ ID NO: 951)
SPHKSR,
(SEQ ID NO: 1415)
SPHKSW,
(SEQ ID NO: 1416)
SPHKSN,
(SEQ ID NO: 1417)
SPHKHG,
(SEQ ID NO: 966)
SPHKYG,
(SEQ ID NO: 1418)
SPHKSQ,
(SEQ ID NO: 4650)
SPHKIV,
(SEQ ID NO: 1419)
SPHKSK,
(SEQ ID NO: 1420)
SPHKLW,
(SEQ ID NO: 1422)
SPHKMG,
or
(SEQ ID NO: 1423)
SPHKMA;

(ii) an amino acid sequence comprising any portion of an amino acid sequence in (i), e.g., any 2, 3, 4, or 5 amino acids, e.g., consecutive amino acids, thereof;
(iii) an amino acid sequence comprising one, two, or three but no more than four modifications, e.g., substitutions (e.g., conservative substitutions), insertions, or deletions, relative to any of the amino acid sequences in (i); or
(iv) an amino acid sequence comprising one, two, or three but no more than four different amino acids, relative to any one of the amino acid sequences in (i).
173. The AAV particle of any one of embodiments 165-172, wherein [N2]-[N3] is or comprises SPHKSG (SEQ ID NO: 946).
174. The AAV particle of any one of embodiments 165-173, wherein the AAV capsid variant comprises an amino acid other than G at position 453 (e.g., A, K, W, R, L, I, M, N, T, E, Q, Y, H, F, or V), numbered according to SEQ ID NO: 138 or 981.
175. The AAV particle of any one of embodiments 165-173, wherein the AAV capsid variant comprises the amino acid G at position 453, numbered according to SEQ ID NO: 138 or 981.
176. The AAV particle of any one of embodiments 165-175, wherein:
   (i) position X1 of [N1] is G, A, K, W, R, L, I, M, N, T, E, Q, Y, H, F, or V;
   (ii) position X2 of [N1] is H, Y, R, Q, N, P, or D;
   (iii) position X3 of [N1] is D, E, G, V, or N;
   optionally wherein the AAV capsid variant comprises an amino acid modification, e.g., a conservative substitution, of any of the aforesaid amino acids in (i), (ii), or (iii).
177. The AAV particle of any one of embodiments 165-176, wherein position X2 of [N1] is H and position X3 of [N1] is D.
178. The AAV particle of any one of embodiments 165-177, wherein position X1 of [N1] is G, position X2 of [N1] is H and position X3 of [N1] is D.
179. The AAV particle of any one of embodiments 165-178, wherein [N1] comprises GH, HD, GY, GR, GQ, AH, GN, KH, GP, WH, RH, LH, IH, MH, GD, NH, TH, EH, QH, YH, HH, FH, VH, YD, HE, RG, QD, RD, ND, PD, QV, DD, HN, or NG
180. The AAV particle of any one of embodiments 165-179, wherein [N1] is or comprises GHD, GYD, GHE, GRG, GOD, GRD, AHD, GND, KHD, GPD, WHD, RHD, LHD, GQV, IHD, MHD, GDD, GHN, NHD, THD, GNG, EHD, QHD, YHD, HHD, FHD, or VHD.
181. The AAV particle of any one of embodiments 165-180, wherein [N1] is or comprises GHD.
182. The AAV particle of any one of embodiments 165-181, wherein [N1]-[N2] comprises HDSPH (SEQ ID NO: 1374).
183. The AAV particle of any one of embodiments 165-182, wherein [N1]-[N2] is or comprises:

(i)
(SEQ ID NO: 1457)
GHDSPH,
(SEQ ID NO: 1502)
GYDSPH,
(SEQ ID NO: 1466)
GHESPH,
(SEQ ID NO: 1461)
GRGSPH,
(SEQ ID NO: 4651)
GHDNPH,
(SEQ ID NO: 1458)
GODSPH,
(SEQ ID NO: 1504)
GRDSPH,
(SEQ ID NO: 4652)
AHDSPH,
(SEQ ID NO: 1505)
GNDSPH,
(SEQ ID NO: 4653)
KHDSPH,
(SEQ ID NO: 1506)
GPDSPH,
(SEQ ID NO: 4654)
WHDSPH,
(SEQ ID NO: 4655)
RHDSPH,
(SEQ ID NO: 4656)
LHDSPH,
(SEQ ID NO: 1508)
GQVSPH,
(SEQ ID NO: 4657)
IHDSPH,
(SEQ ID NO: 4658)
MHDSPH,
(SEQ ID NO: 1465)
GDDSPH,
(SEQ ID NO: 1509)
GHNSPH,
(SEQ ID NO: 4659)
NHDSPH,
(SEQ ID NO: 4660)
THDSPH,
(SEQ ID NO: 1478)
GNGSPH,
(SEQ ID NO: 4661)
EHDSPH,
(SEQ ID NO: 4662)
QHDSPH,
(SEQ ID NO: 4663)
YHDSPH,
(SEQ ID NO: 4664)
HHDSPH,
(SEQ ID NO: 4665)
FHDSPH,
or
(SEQ ID NO: 4666)
VHDSPH;

(ii) an amino acid sequence comprising any portion of an amino acid sequence in (i), e.g., any 2, 3, 4, or 5 amino acids, e.g., consecutive amino acids, thereof;
(iii) an amino acid sequence comprising one, two, or three but no more than four modifications, e.g., substitutions (e.g., conservative substitutions), insertions, or deletions, relative to any of the amino acid sequences in (i); or
(iv) an amino acid sequence comprising one, two, or three but no more than four different amino acids, relative to any one of the amino acid sequences in (i).
184. The AAV particle of any one of embodiments 165-183, wherein [N1]-[N2]-[N3] is or comprises:

(i)
(SEQ ID NO: 1370)
GHDSPHKSG,
(SEQ ID NO: 1669)
GHDSPHKIG,
(SEQ ID NO: 1670)
GYDSPHKSG,
(SEQ ID NO: 1671)
GHESPHKSG,
(SEQ ID NO: 1672)
GHDSPHKTG,
(SEQ ID NO: 1673)
GRGSPHKRG,
(SEQ ID NO: 4667)
GHDNPHKSG,
(SEQ ID NO: 1581)
GQDSPHKSG,
(SEQ ID NO: 1613)
GHDSPHKSA,
(SEQ ID NO: 1674)
GHDSPHKSL,
(SEQ ID NO: 1676)
GHDSPHKSE,
(SEQ ID NO: 1677)
GRDSPHKSG,
(SEQ ID NO: 4668)
AHDSPHKSG,
(SEQ ID NO: 1678)
GNDSPHKSV,
(SEQ ID NO: 4669)
AHDSPHKIG,
(SEQ ID NO: 1612)
GHESPHKSA,
(SEQ ID NO: 1679)
GQDSPHKIG,
(SEQ ID NO: 1680)
GHDSPHKSV,
(SEQ ID NO: 1615)
GHDSPHKSR,
(SEQ ID NO: 4670)
KHDSPHKSG,
(SEQ ID NO: 1681)
GPDSPHKIG,
(SEQ ID NO: 1682)
GPDSPHKSG,
(SEQ ID NO: 1683)
GHDSPHKSW,
(SEQ ID NO: 4671)
WHDSPHKSG,
(SEQ ID NO: 4672)
RHDSPHKSG,
(SEQ ID NO: 1684)
GHDSPHKSN,
(SEQ ID NO: 1610)
GHDSPHKRG,
(SEQ ID NO: 1686)
GHDSPHKHG,
(SEQ ID NO: 4673)
LHDSPHKSG,
(SEQ ID NO: 1687)
GQVSPHKSG,
(SEQ ID NO: 4674)
IHDSPHKSG,
(SEQ ID NO: 4675)
MHDSPHKSG,
(SEQ ID NO: 1688)
GDDSPHKSV,
(SEQ ID NO: 1689)
GHNSPHKSG,
(SEQ ID NO: 4676)
NHDSPHKSG,
(SEQ ID NO: 4677)
THDSPHKSG,
(SEQ ID NO: 1690)
GNGSPHKRG,
(SEQ ID NO: 4678)
EHDSPHKSG,
(SEQ ID NO: 1691)
GHDSPHKYG,
(SEQ ID NO: 1692)
GHDSPHKSQ,
(SEQ ID NO: 4680)
QHDSPHKSG,
(SEQ ID NO: 4681)
RHDSPHKIV,
(SEQ ID NO: 4682)
YHDSPHKSG,
(SEQ ID NO: 1693)
GNDSPHKIG,
(SEQ ID NO: 4683)
HHDSPHKSG,
(SEQ ID NO: 1694)
GHDSPHKSK,
(SEQ ID NO: 4684)
FHDSPHKSG,
(SEQ ID NO: 1695)
GHDSPHKLW,
(SEQ ID NO: 4685)
VHDSPHKSG,
(SEQ ID NO: 1697)
GHDSPHKMG,
(SEQ ID NO: 1698)
GHDSPHKMA,
or
(SEQ ID NO: 1611)
GDDSPHKSG;

(ii) an amino acid sequence comprising any portion of an amino acid sequence in (i), e.g., any 2, 3, 4, 5, 6, 7, 8, or 9 amino acids, e.g., consecutive amino acids, thereof;
(iii) an amino acid sequence comprising one, two, or three but no more than four modifications, e.g., substitutions (e.g., conservative substitutions), insertions, or deletions, relative to any of the amino acid sequences in (i); or
(iv) an amino acid sequence comprising one, two, or three but no more than four different amino acids, relative to any one of the amino acid sequences in (i).
185. The AAV particle of any one of embodiments 165-184, wherein [N1]-[N2]-[N3] is or comprises GHDSPHKSG (SEQ ID NO: 1370).
186. The AAV particle of any one of embodiments 165-185, wherein the AAV capsid variant comprises an amino acid other than Q at position 456 (e.g., R, P, H, K, L, V, A, E, or I), an amino acid other than N at position 457 (e.g., I, K, S, H, R, T, D, Y, L, W, F, A, Q, or M), an amino acid other than Q at position 458 (e.g., R, V, K, P, Y, H, L, I, E, or M), an amino acid other than Q at position 459 (e.g., H, L, E, P, W, D, I, V, S, K, R, C, M, or N), and/or an amino acid other than T at position 460 (e.g., A, E, K, S, I, P, G, or N), relative to a reference sequence numbered according to SEQ ID NO: 138.
187. The AAV particle of any one of embodiments 165-186, wherein the AAV capsid variant comprises an amino acid other than Q at position 462 (e.g., R, P, H, K, L, V, A, E, or I), an amino acid other than N at position 463 (e.g., I, K, S, H, R, T, D, Y, L, W, F, A, Q, or M), an amino acid other than Q at position 464 (e.g., R, V, K, P, Y, H, L, I, E, or M), an amino acid other than Q at position 465 (e.g., H, L, E, P, W, D, I, V, S, K, R, C, M, or N), and/or an amino acid other than T at position 466 (e.g., A, E, K, S, I, P, G, or N), relative to a reference sequence numbered according to SEQ ID NO: 982.
188. The AAV particle of any one of embodiments 165-187, wherein the AAV capsid variant comprises the amino acid Q at position 456, the amino acid N at position 457, the amino acid Q at position 458, the amino acid Q at position 459, and/or the amino acid T at position 460, relative to a reference sequence numbered according to SEQ ID NO: 138.
189. The AAV particle of any one of embodiments 165-188, wherein the AAV capsid variant comprises the amino acid Q at position 462, the amino acid N at position 463, the amino acid Q at position 464, the amino acid Q at position 465, and/or the amino acid T at position 466, relative to a reference sequence numbered according to SEQ ID NO: 982.
190. The AAV particle of any one of embodiments 165-189, wherein the AAV capsid variant further comprises [N4] wherein [N4] comprises X7, X8, X9, X10, and X11, wherein:
   (a) X7 is Q, R, P, H, L, K, I, G, S, M, or E;
   (b) X8 is N, D, V, S, P, T, G, Y, W, E, R, H, K, F, A, I, L, or M;
   (c) X9 is Q, R, L, A, P, H, T, I, F, K, V, M, G, W, Y, S, E, N, D;
   (d) X10 is Q, H, K, A, L, P, E, M, I, S, N, R, Y, C, V, T, W, D, G; and
   (e) X11 is T, I, N, S, H, R, L, D, Y, A, Q;
   optionally wherein the AAV capsid variant comprises an amino acid modification, e.g., a conservative substitution, of any of the aforesaid amino acids in (a)-(e).
191. The AAV particle of embodiment 190, wherein [N4] is or comprises:

(i)
(SEQ ID NO: 3964)
QNQQT,
(SEQ ID NO: 4686)
QIRQT,
(SEQ ID NO: 4687)
QNQHA,
(SEQ ID NO: 4530)
QKQQT,
(SEQ ID NO: 4688)
QSVQT,
(SEQ ID NO: 4689)
RSQQT,
(SEQ ID NO: 4690)
QNKLE,
(SEQ ID NO: 4691)
QNQQK,
(SEQ ID NO: 4692)
QHQQA,
(SEQ ID NO: 4693)
QIQHT,
(SEQ ID NO: 4694)
PRQQT,
(SEQ ID NO: 4695)
HTQQT,
(SEQ ID NO: 4696)
QRQHT,
(SEQ ID NO: 3970)
QSQQT,
(SEQ ID NO: 4697)
QNQQS,
(SEQ ID NO: 4698)
RNQET,
(SEQ ID NO: 4075)
QTQLT,
(SEQ ID NO: 4699)
KNQQT,
(SEQ ID NO: 3984)
QDQQT,
(SEQ ID NO: 3978)
HNQQT,
(SEQ ID NO: 3975)
QNQLT,
(SEQ ID NO: 4700)
QTQQT,
(SEQ ID NO: 4701)
QTQQI,
(SEQ ID NO: 4702)
QSKQA,
(SEQ ID NO: 4703)
QNQPP,
(SEQ ID NO: 4704)
QSPQT,
(SEQ ID NO: 4091)
QNYQT,
(SEQ ID NO: 3983)
QNHQT,
(SEQ ID NO: 3998)
QNRQT,
(SEQ ID NO: 4705)
QNQQG,
(SEQ ID NO: 4079)
QNHLT,
(SEQ ID NO: 3999)
QYQHT,
(SEQ ID NO: 4490)
QNQWT,
(SEQ ID NO: 4706)
QNQHT,
(SEQ ID NO: 4707)
QTRQT,
(SEQ ID NO: 4708)
QNLHT,
(SEQ ID NO: 4068)
LNQQT,
(SEQ ID NO: 4709)
QNQET,
(SEQ ID NO: 4500)
QHLQT,
(SEQ ID NO: 4710)
LNQPT,
(SEQ ID NO: 4711)
QNQDT,
(SEQ ID NO: 4712)
RNQQT,
(SEQ ID NO: 4713)
QNLLT,
(SEQ ID NO: 4714)
QLVIT,
(SEQ ID NO: 4715)
RTQET,
(SEQ ID NO: 4716)
QTHQT,
(SEQ ID NO: 4717)
QNQPA,
(SEQ ID NO: 4718)
QDQHT,
(SEQ ID NO: 4719)
QSQHT,
(SEQ ID NO: 4720)
RNQQI,
(SEQ ID NO: 4721)
VRQQT,
(SEQ ID NO: 4722)
QNQHS,
(SEQ ID NO: 4723)
AWQQT,
(SEQ ID NO: 4724)
QSVPT,
(SEQ ID NO: 4725)
QNIQP,
(SEQ ID NO: 4726)
QNHLN,
(SEQ ID NO: 4727)
LDQQT,
(SEQ ID NO: 4728)
PDQQS,
(SEQ ID NO: 4729)
ESQQT,
(SEQ ID NO: 4730)
QNKQT,
(SEQ ID NO: 4731)
QRQLT,
(SEQ ID NO: 4732)
QIIVT,
(SEQ ID NO: 4733)
QKQST,
(SEQ ID NO: 4734)
QSHQT,
(SEQ ID NO: 4735)
QFVVT,
(SEQ ID NO: 3980)
QNLQT,
(SEQ ID NO: 3971)
QNQQI,
(SEQ ID NO: 4736)
QSQPT,
(SEQ ID NO: 4737)
QNEQT,
(SEQ ID NO: 4503)
QSLQT,
(SEQ ID NO: 4738)
RNRQT,
(SEQ ID NO: 4739)
QSKQT,
(SEQ ID NO: 4740)
QNPLT,
(SEQ ID NO: 4741)
RDQKT,
(SEQ ID NO: 4742)
HNQQN,
(SEQ ID NO: 4743)
QWKRT,
(SEQ ID NO: 4073)
QSQQI,
(SEQ ID NO: 4058)
QAQQT,
(SEQ ID NO: 4744)
QNHQI,
(SEQ ID NO: 4745)
QNQQA,
(SEQ ID NO: 4746)
QNQLN,
(SEQ ID NO: 4747)
QTQPT,
(SEQ ID NO: 4547)
INQQT,
(SEQ ID NO: 4748)
QKQLT,
(SEQ ID NO: 4749)
RNQLA,
(SEQ ID NO: 4750)
RNQQS,
(SEQ ID NO: 4751)
ISIQT,
(SEQ ID NO: 4752)
QNQQN,
(SEQ ID NO: 4753)
QSQQS,
(SEQ ID NO: 4754)
QTVCT,
(SEQ ID NO: 4755)
QYQQI,
(SEQ ID NO: 4756)
QQIMT,
(SEQ ID NO: 4757)
QNEQS,
(SEQ ID NO: 4758)
LNHQT,
(SEQ ID NO: 4759)
QMIHT,
(SEQ ID NO: 4760)
RNHQS,
(SEQ ID NO: 4761)
QKMNT,
(SEQ ID NO: 4762)
QSQQN,
(SEQ ID NO: 4061)
QYQHA;

(ii) an amino acid sequence comprising any portion of an amino acid sequence in (i), e.g., any 2, 3, or 4 amino acids, e.g., consecutive amino acids, thereof;
(iii) an amino acid sequence comprising one, two, or three but no more than four modifications, e.g., substitutions (e.g., conservative substitutions), insertions, or deletions, relative to any of the amino acid sequences in (i); or
(iv) an amino acid sequence comprising one, two, or three but no more than four different amino acids, relative to any one of the amino acid sequences in (i).
192. The AAV particle of embodiment 190 or 191, wherein [N1]-[N2]-[N3]-[N4] is or comprises:
   (i) the amino acid sequence of any of SEQ ID NOs: 201 or 3160-3237;
   (ii) an amino acid sequence comprising any portion of an amino acid sequence in (i), e.g., any 2, 3, 4, 5, 6, 7, 8, 9, 10, 11, 12, or 13 amino acids, e.g., consecutive amino acids, thereof;
   (iii) an amino acid sequence comprising one, two, or three but no more than four modifications, e.g., substitutions (e.g., conservative substitutions), insertions, or deletions, relative to any of the amino acid sequences in (i); or
   (iv) an amino acid sequence comprising one, two, or three but no more than four different amino acids, relative to any one of the amino acid sequences in (i).
193. The AAV particle of any one of embodiments 190-192, wherein [N1]-[N2]-[N3]-[N4] is or comprises GHDSPHKSGQNQQT (SEQ ID NO: 201).
194. The AAV particle of any one of embodiments 165-193, wherein the AAV capsid variant comprises an amino acid other than K at position 449 (e.g., T), T at position 450 (e.g., A, S, I, V, N, E, Y, C, G, W, or Q), an amino acid other than I at position 451 (e.g., E, V, S, T, N, D, C, G, Q, L, P, A), and/or an amino acid other than N at position 452 (e.g., S, Y, I, K, F, T, D, E, G, V, L, A, M, Q, H, P, or R), relative to a reference sequence numbered according to SEQ ID NO: 138 or 982.
195. The AAV particle of any one of embodiments 165-193, wherein the AAV capsid variant comprises the amino acid K at position 449, the amino acid T at position 450, the amino acid I at position 451, and/or the amino acid N at position 452, relative to a reference sequence numbered according to SEQ ID NO: 138.
196. The AAV particle of any one of embodiments 165-195, wherein the AAV capsid variant further comprises [N0], wherein [N0] comprises X_{A}, X_{B}, X_{C}, and X_{D}, wherein:
   (a) X_{A} is K or T;
   (b) X_{b} is T, A, S, I, V, N, E, Y, C, G, W, or Q;
   (c) X_{C} is I, E, V, S, T, N, D, C, G, Q, L, P, A; and
   (d) X_{D} is N, S, Y, I, K, F, T, D, E, G, V, L, A, M, Q, H, P, or R;
   optionally wherein the AAV capsid variant comprises an amino acid modification, e.g., a conservative substitution, of any of the aforesaid amino acids in (a)-(d).
197. The AAV particle of embodiment 196, wherein [N0] is or comprises:

(i)
(SEQ ID NO: 4562)
KAIN,
(SEQ ID NO: 4557)
KTIN,
(SEQ ID NO: 4588)
KTES,
(SEQ ID NO: 4558)
TTIN,
(SEQ ID NO: 4559)
KSIN,
(SEQ ID NO: 4555)
KTVN,
(SEQ ID NO: 4763)
KSIY,
(SEQ ID NO: 4565)
KTSN,
(SEQ ID NO: 4589)
KTTN,
(SEQ ID NO: 4764)
KIIN,
(SEQ ID NO: 4593)
KTIS,
(SEQ ID NO: 4765)
KAII,
(SEQ ID NO: 4599)
KTIK,
(SEQ ID NO: 4611)
KTEF,
(SEQ ID NO: 4607)
KTIT,
(SEQ ID NO: 4591)
KTNN,
(SEQ ID NO: 4579)
KTID,
(SEQ ID NO: 4766)
KAIS,
(SEQ ID NO: 4767)
KTVD,
(SEQ ID NO: 4768)
KTIE,
(SEQ ID NO: 4634)
KTEG,
(SEQ ID NO: 4610)
KVIN,
(SEQ ID NO: 4632)
KAVN,
(SEQ ID NO: 4769)
KTIY,
(SEQ ID NO: 4563)
KTDN,
(SEQ ID NO: 4570)
KTCN,
(SEQ ID NO: 4770)
KNVV,
(SEQ ID NO: 4582)
KTEL,
(SEQ ID NO: 4771)
KTDA,
(SEQ ID NO: 4631)
KTEV,
(SEQ ID NO: 4629)
KSEL,
(SEQ ID NO: 4772)
KTEM,
(SEQ ID NO: 4619)
KTEQ,
(SEQ ID NO: 4552)
KTII,
(SEQ ID NO: 4773)
KIVN,
(SEQ ID NO: 4554)
KTEK,
(SEQ ID NO: 4568)
KTEN,
(SEQ ID NO: 4774)
KIGN,
(SEQ ID NO: 4775)
KEVM,
(SEQ ID NO: 4776)
KYQV,
(SEQ ID NO: 4584)
KTEA,
(SEQ ID NO: 4573)
KATN,
(SEQ ID NO: 4571)
KTEH,
(SEQ ID NO: 4777)
KTVE,
(SEQ ID NO: 4778)
KAID,
(SEQ ID NO: 4779)
KTIM,
(SEQ ID NO: 4780)
KEVG,
(SEQ ID NO: 4781)
KSEM,
(SEQ ID NO: 4782)
KAQQ,
(SEQ ID NO: 4783)
KCGE,
(SEQ ID NO: 4784)
KASN,
(SEQ ID NO: 4581)
KTET,
(SEQ ID NO: 4785)
KTIG,
(SEQ ID NO: 4786)
KTDP,
(SEQ ID NO: 4787)
KELV,
(SEQ ID NO: 4788)
KELM,
(SEQ ID NO: 4789)
KNEI,
(SEQ ID NO: 4790)
KTPN,
(SEQ ID NO: 4791)
KITN,
(SEQ ID NO: 4792)
KTDI,
(SEQ ID NO: 4793)
KTDQ,
(SEQ ID NO: 4794)
KGIN,
(SEQ ID NO: 4795)
KSEI,
(SEQ ID NO: 4614)
KSEK,
(SEQ ID NO: 4796)
KWSA,
(SEQ ID NO: 4797)
KELA,
(SEQ ID NO: 4798)
KQTQ,
(SEQ ID NO: 4799)
KGAD,
(SEQ ID NO: 4800)
KVGE,
(SEQ ID NO: 4801)
KANE,
(SEQ ID NO: 4802)
KTDT,
(SEQ ID NO: 4803)
KTCI,
(SEQ ID NO: 4804)
KELR,
(SEQ ID NO: 4805)
KCQI,
(SEQ ID NO: 4806)
KGVM,
(SEQ ID NO: 4807)
KACD,
(SEQ ID NO: 4808)
KNEL,
(SEQ ID NO: 4809)
KAAE,
(SEQ ID NO: 4810)
KGQN,
(SEQ ID NO: 4811)
KNEF,
(SEQ ID NO: 4812)
KTSI,
(SEQ ID NO: 4603)
KAEH,
(SEQ ID NO: 4813)
KCDQ,
(SEQ ID NO: 4814)
KEIL,
(SEQ ID NO: 4560)
KTER,
(SEQ ID NO: 4637)
KNAI,
(SEQ ID NO: 4575)
KTDK,
(SEQ ID NO: 4815)
KTPD,
(SEQ ID NO: 4646)
KTIH,
or
(SEQ ID NO: 4578)
KTEI

(ii) an amino acid sequence comprising any portion of an amino acid sequence in (i), e.g., any 2, or 3 amino acids, e.g., consecutive amino acids, thereof;
(iii) an amino acid sequence comprising one, two, or three but no more than four modifications, e.g., substitutions (e.g., conservative substitutions), insertions, or deletions, relative to any of the amino acid sequences in (i); or
(iv) an amino acid sequence comprising one, two, or three but no more than four different amino acids, relative to any one of the amino acid sequences in (i).
198. The AAV particle of embodiment 196 or 197, wherein [N0]-[N1]-[N2]-[N3]-[N4] is or comprises:
   (i) the amino acid sequence of any one of SEQ ID NOs: 3606-3836;
   (ii) an amino acid sequence comprising any portion of an amino acid sequence in (i), e.g., any 2, 3, 4, 5, 6, 7, 8, 9, 10, 11, 12, 13, 14, 15, 16, or 17 amino acids, e.g., consecutive amino acids, thereof;
   (iii) an amino acid sequence comprising one, two, or three but no more than four modifications, e.g., substitutions (e.g., conservative substitutions), insertions, or deletions, relative to any of the amino acid sequences in (i); or
   (iv) an amino acid sequence comprising one, two, or three but no more than four different amino acids, relative to any one of the amino acid sequences in (i).
199. The AAV particle of any one of embodiments 196-198, wherein [N0]-[N1]-[N2]-[N3]-[N4] is or comprises KTINGHDSPHKSGQNQQT (SEQ ID NO: 5828).
200. The AAV particle of any one of embodiments 196-198, wherein [N0]-[N1]-[N2]-[N3]-[N4] is or comprises KAEIGHDSPHKSGQNQQT (SEQ ID NO: 1754).
201. The AAV particle of any one of embodiments 196-198, wherein [N0]-[N1]-[N2]-[N3]-[N4] is or comprises KTEKMSGSPHSKAQNQQT (SEQ ID NO: 3241).
202. The AAV particle of any one of embodiments 130-200, wherein [N1]-[N2]-[N3] is present in loop IV of the AAV capsid variant.
203. The AAV particle of any one of embodiments 160-164 or 196-202, wherein [N0] and [N4] are present in loop IV of the AAV capsid variant.
204. The AAV particle of any one of embodiments 160-164 or 196-203, wherein [N0] is present immediately subsequent to position 448, relative to a reference sequence numbered according to the amino acid sequence of SEQ ID NO: 138, 981, or 982.
205. The AAV particle of any one of embodiments 160-164 or 196-204, wherein [N0] replaces positions 449-452 (e.g., K449, T450, 1451, and N452), relative to a reference sequence numbered according to SEQ ID NO: 138, 981, or 982.
206. The AAV particle of any one of embodiments 160-164 or 196-205, wherein [N0] is present immediately subsequent to position 448 and wherein [N0] replaces positions 449-452 (e.g., K449, T450, I451, and N452), relative to a reference sequence numbered according to SEQ ID NO: 138, 981, or 982.
207. The AAV particle of any one of embodiments 160-164 or 196-206, wherein [N0] corresponds to positions 449-452 (e.g., K449, T450, 1451, and N452) of SEQ ID NO: 981 or 982.
208. The AAV particle of any one of embodiments 130-207, wherein [N1] is present immediately subsequent to position 452, relative to a reference sequence numbered according to the amino acid sequence of SEQ ID NO: 138, 981, or 982.
209. The AAV particle of any one of embodiments 130-208, wherein [N1] replaces positions 453-455 (e.g., G453, S454, and G455), relative to a reference sequence numbered according to SEQ ID NO: 138, 981, or 982.
210. The AAV particle of any one of embodiments 130-208, wherein [N1] replaces position 453 (e.g., G453), relative to a reference sequence numbered according to SEQ ID NO: 138, 981, or 982.
211. The AAV particle of any one of embodiments 130-164 or 200-209, wherein:
   (i) position X1 of [N1] replaces position 453 (e.g., G453);
   (ii) position X2 of [N1] corresponds to position 454 (e.g., S454); and
   (iii) position X3 of [N1] corresponds to position 455 (e.g., G455), wherein (i), (ii), and (iii) are numbered according to SEQ ID NO: 138 or SEQ ID NO: 981.
212. The AAV particle of any one of embodiments 130-164 or 200-209, wherein:
   (i) position X1 of [N1] corresponds to position 453 (e.g., G453);
   (ii) position X2 of [N1] corresponds to position 454 (e.g., S454); and
   (iii) position X3 of [N1] corresponds to position 455 (e.g., G455), wherein (i), (ii), and (iii) are numbered according to SEQ ID NO: 138 or SEQ ID NO: 981.
213. The AAV particle of any one of embodiments 165-209, wherein:
   (i) position X1 of [N1] corresponds to position 453 (e.g., G453);
   (ii) position X2 of [N1] replaces position 454 (e.g., S454); and
   (iii) position X3 of [N1] replaces position 455 (e.g., G455);
   wherein (i), (ii), and (iii) are numbered according to SEQ ID NO: 138 or SEQ ID NO: 982.
214. The AAV particle of any one of embodiments 165-209, wherein [N1] corresponds to positions 453-455 (e.g., G453, H454, D455) of SEQ ID NO 982.
215. The AAV particle of any one of embodiments 165-209, wherein [N1] corresponds to positions 453-455 (e.g., G453, S454, G455) of SEQ ID NO: 138 or 981.
216. The AAV particle of any one of embodiments 130-215, wherein [N2] is present immediately subsequent to position 455, relative to a reference sequence numbered according to the amino acid sequence of SEQ ID NO: 138, 981, or 982.
217. The AAV particle of any one of embodiments 130-216, wherein [N2] corresponds to positions 456-458 (e.g., S456, P457, and H458) of SEQ ID NO: 981 or 982.
218. The AAV particle of any one of embodiments 130-216, wherein [N2] corresponds to positions 456-458 (e.g., S456, P457, and H458) of any one of SEQ ID NOs: 69, 70, 71, 4, 5, 6, 7, 8, or 36-59.
219. The AAV particle of any one of embodiments 130-218, wherein [N2] is present immediately subsequent to [N1].
220. The AAV particle of any one of embodiments 130-219, wherein [N3] corresponds to positions 459-460 (e.g., S459, K460, A461) of SEQ ID NO 981.
221. The AAV particle of any one of embodiments 130-220, wherein [N3] corresponds to positions 459-460 (e.g., S459, K460, A461) of SEQ ID NO: 69, 36, 38, 39, 40, 41, 42, 43, 44, 45, 46, 47, 48, 49, 50, 51, 52, 53, 54, 55, 57, or 59
222. The AAV particle of any one of embodiments 130-220, wherein [N2]-[N3] is present immediately subsequent to position 455, relative to a reference sequence numbered according to the amino acid sequence of SEQ ID NO: 138, 981, or 982.
223. The AAV particle of any one of embodiments 130-164 or 200-222, wherein [N2]-[N3] is present immediately subsequent to position 455, relative to a reference sequence numbered according to the amino acid sequence of SEQ ID NO: 981.
224. The AAV particle of any one of embodiments 130-164 or 200-223, wherein [N2]-[N3] corresponds to positions 456-461 (e.g., S456, P457, H458, S459, K460, A461) of SEQ ID NO: 981.
225. The AAV particle of any one of embodiments 130-164 or 200-223, wherein [N2]-[N3] corresponds to positions 456-461 (e.g., S456, P457, H458, S459, K460, A461) of any one of SEQ ID NOs: 69, 36, 38, 39, 40, 41, 42, 43, 44, 45, 46, 47, 48, 49, 50, 51, 52, 53, 54, 55, 57, or 59.
226. The AAV particle of any one of embodiments 165-222, wherein [N3] corresponds to positions 459-460 (e.g., K459, S460, G461) of SEQ ID NO: 982.
227. The AAV particle of any one of embodiments 165-219 or 222-226, wherein [N3] corresponds to positions 459-460 (e.g., K459, S460, G461) of SEQ ID NO: 37.
228. The AAV particle of any one of embodiments 165-222 or 227, wherein [N2]-[N3] is present immediately subsequent to position 455, relative to a reference sequence numbered according to the amino acid sequence of SEQ ID NO: 982.
229. The AAV particle of any one of embodiments 165-228, wherein [N3] replaces positions 454 and 455 (e.g., S454 and G455), numbered according to SEQ ID NO: 138.
230. The AAV particle of any one of embodiments 165-229, wherein [N3] is present immediately subsequent to [N2] and replaces positions 454 and 455 (e.g., S454 and G455), numbered according to SEQ ID NO: 138.
231. The AAV particle of any one of embodiments 165-230, wherein [N3] is present immediately subsequent to [N1]-[N2] and replaces positions 454 and 455 (e.g., S454 and G455), numbered according to SEQ ID NO: 138.
232. The AAV particle of any one of embodiments 165-222, 226, or 228, wherein [N2]-[N3] corresponds to positions 456-461 (e.g., S456, P457, H458, K459, S460, G461) of SEQ ID NO: 982.
233. The AAV particle of any one of embodiments 165-222, 226, or 228, wherein [N2]-[N3] corresponds to positions 456-461 (e.g., S456, P457, H458, K459, S460, G461) of SEQ ID NO: 37.
234. The AAV particle of any one of embodiments 153-164 or 190-233, wherein [N4] is present immediately subsequent to position 455, relative to a reference sequence numbered according to the amino acid sequence of SEQ ID NO: 138.
235. The AAV particle of any one of embodiments 153-164 or 190-234, wherein [N4] replaces positions 456-460 (e.g., Q456, N457, Q458, Q459, and T460), relative to a reference sequence numbered according to the amino acid sequence of SEQ ID NO: 138.
236. The AAV particle of any one of embodiments 153-164 or 190-235, wherein [N4] corresponds to positions 462-466 (e.g., Q462, N463, Q464, Q465, and T466) of SEQ ID NO: 981 or 982.
237. The AAV particle of any one of embodiments 153-164 or 190-235, wherein [N4] corresponds to positions 462-466 of any one of SEQ ID NOs: 69, 70, 71, 4, 5, 6, 7, 8, or 36-59.
238. The AAV particle of any one of embodiments 153-164 or 190-235, wherein [N4] corresponds to positions 456-460 (e.g., Q456, N457, Q458, Q459, and T460) of SEQ ID NO: 138.
239. The AAV particle of any one of embodiments 153-164 or 190-236, wherein [N2]-[N3]-[N4] replaces positions 456-460 (e.g., Q456, N457, Q458, Q459, and T460), relative to a reference sequence numbered according to the amino acid sequence of SEQ ID NO: 138.
240. The AAV particle of any one of embodiments 153-164 or 190-239, wherein [N2]-[N3]-[N4] is present immediately subsequent to position 455, and wherein [N2]-[N3]-[N4] replaces positions 456-460 (e.g., Q456, N457, Q458, Q459, and T460), relative to a reference sequence numbered according to the amino acid sequence of SEQ ID NO: 138.
241. The AAV particle of any one of embodiments 153-164 or 190-240, wherein [N1]-[N2]-[N3]-[N4] replaces positions 453-460 (e.g., G453, S454, G455, Q456, N457, Q458, Q459, and T460), relative to a reference sequence numbered according to the amino acid sequence of SEQ ID NO: 138.
242. The AAV particle of any one of embodiments 153-164 or 190-241, wherein [N1]-[N2]-[N3]-[N4] is present immediately subsequent to position 452, and wherein [N1]-[N2]-[N3]-[N4] replaces positions 453-460 (e.g., G453, S454, G455, Q456, N457, Q458, Q459, and T460), relative to a reference sequence numbered according to the amino acid sequence of SEQ ID NO: 138.
243. The AAV particle of any one of embodiments 153-164 or 190-242, wherein [N1]-[N2]-[N3]-[N4] corresponds to positions 453-466 (e.g., G453, S454, G455, S456, P457, H458, S459, K460, A461, Q462, N463, Q464, Q465, T466) of SEQ ID NO: 981.
244. The AAV particle of any one of embodiments 153-164 or 190-243, wherein [N1]-[N2]-[N3] corresponds to positions 453-461 (e.g., G453, S454, G455, S456, P457, H458, S459, K460, A461) of SEQ ID NO: 981.
245. The AAV particle of any one of embodiments 153-164 or 190-242, wherein [N1]-[N2]-[N3]-[N4] corresponds to positions 453-466 (e.g., G453, H454, D455, S456, P457, H458, K459, S460, G461, Q462, N463, Q464, Q465, T466) of SEQ ID NO: 982.
246. The AAV particle of any one of embodiments 153-164, 190-242, or 245, wherein [N1]-[N2]-[N3] corresponds to positions 453-461 (e.g., G453, H454, D455, S456, P457, H458, K459, S460, G461) of SEQ ID NO: 982.
247. The AAV particle of any one of embodiments 153-164, 190-242, or 245, wherein [N1]-[N2]-[N3]-[N4] corresponds to positions 453-466 of any one of SEQ ID NOs: 69, 70, 71, 4, 5, 6, 7, 8, or 36-59.
248. The AAV particle of any one of embodiments 160-164 or 196-245, wherein [N0]-[N1]-[N2]-[N3]-[N4] replaces positions 449-460 (e.g., K449, T450, I451, N452, G453, S454, G455, Q456, N457, Q458, Q459, T460), relative to a reference sequence numbered according to the amino acid sequence of SEQ ID NO: 138.
249. The AAV particle of any one of embodiments 160-164 or 196-248, wherein [N0]-[N1]-[N2]-[N3]-[N4] is present immediately subsequent to position 448, and wherein [N0]-[N1]-[N2]-[N3]-[N4] replaces positions 449-460 (e.g., K449, T450, 1451, N452, G453, S454, G455, Q456, N457, Q458, Q459, T460), relative to a reference sequence numbered according to the amino acid sequence of SEQ ID NO: 138.
250. The AAV particle of any one of embodiments 160-164 or 202-249, wherein [N0]-[N1]-[N2]-[N3]-[N4] corresponds to positions 449-466 (e.g., K449, T450, 1451, N452, G453, S454, G455, S456, P457, H458, S459, K460, A461, Q462, N463, Q464, Q465, T466) of SEQ ID NO: 981.
251. The AAV particle of any one of embodiments 202-249, wherein [N0]-[N1]-[N2]-[N3]-[N4] corresponds to positions 449-466 (e.g., K449, T450, 1451, N452, G453, H454, D455, S456, P457, H458, K459, S460, G461, Q462, N463, Q464, Q465, T466) of SEQ ID NO: 982.
252. The AAV particle of any one of embodiments 202-249, wherein [N0]-[N1]-[N2]-[N3]-[N4] corresponds to positions 449-466 of any one of SEQ ID NOs: 69, 70, 71, 4, 5, 6, 7, 8, or 36-59.
253. The AAV particle of any one of embodiments 153-164 or 190-251, wherein [N4] is present immediately subsequent to position 461, relative to a reference sequence numbered according to the amino acid sequence of SEQ ID NO: 981 or 982.
254. The AAV particle of any one of embodiments 153-164 or 190-253, wherein [N4] replaces positions 462-466 (e.g., Q462, N463, Q464, Q465, and T466), relative to a reference sequence numbered according to the amino acid sequence of SEQ ID NO: 981 or 982.
255. The AAV particle of any one of embodiments 153-164 or 190-254, wherein [N2]-[N3]-[N4] replaces positions 462-466 (e.g., Q462, N463, Q464, Q465, and T466), relative to a reference sequence numbered according to the amino acid sequence of SEQ ID NO: 981 or 982.
256. The AAV particle of any one of embodiments 153-164 or 190-255, wherein [N2]-[N3]-[N4] is present immediately subsequent to position 455, and wherein [N2]-[N3]-[N4] replaces positions 462-466 (e.g., Q462, N463, Q464, Q465, and T466), relative to a reference sequence numbered according to the amino acid sequence of SEQ ID NO: 981 or 982.
257. The AAV particle of any one of embodiments 130-256, wherein the AAV capsid variant comprises from N-terminus to C-terminus, [N2]-[N3].
258. The AAV particle of any one of embodiments 130-257, wherein the AAV capsid variant comprises from N-terminus to C-terminus, [N1]-[N2]-[N3].
259. The AAV particle of any one of embodiments 130-258, wherein the AAV capsid variant comprises from N-terminus to C-terminus, [N0]-[N1]-[N2]-[N3].
260. The AAV particle of any one of embodiments 130-259, wherein the AAV capsid variant comprises from N-terminus to C-terminus, [N1]-[N2]-[N3]-[N4].
261. The AAV particle of any one of embodiments 130-260, wherein the AAV capsid variant comprises from N-terminus to C-terminus, [N0]-[N1]-[N2]-[N3]-[N4].
262. An AAV particle comprising an AAV capsid variant and a nucleic acid encoding a SOD1 targeting polynucleotide (e.g., a SOD1 targeting RNA agent, e.g., a siRNA), wherein the AAV capsid variant:
   (i) is enriched, e.g., at least about 10, 15, 20, 25, 30, 35, 40, 45, 50, 55, 60, 65, 70, 75, 80, 85, 90, 95, 100, 105, 115, 120, 125, 130, 135, 140, 145, 150, 155, 160, 165, 170, 175, 180, 190, 200, 205, or 210-fold, in the brain of at least two to three species, e.g., a non-human primate and rodent (e.g., mouse), e.g., as compared to a reference sequence of SEQ ID NO: 138, optionally wherein the at least two to three species are Macaca fascicularis, Chlorocebus sabaeus, Callithrix jacchus, and/or mouse (e.g., BALB/c mice, C57BI/6 mice, and/or CD-1 outbred mice); and/or
   (ii) is capable of transducing neuronal cells (e.g., NeuN+neurons or dopaminergic neurons (e.g., tyrosine hydroxylase (TH)+neurons) and non-neuronal cells, e.g., glial cells, oligodendrocytes (e.g., Olig2 positive oligodendrocytes), and/or astrocytes (e.g., Sox9+astrocytes or Olig2 positive astrocytes).
263. An AAV particle comprising an AAV capsid variant and a nucleic acid encoding a SOD1 targeting polynucleotide (e.g., a SOD1 targeting RNA agent, e.g., a siRNA), wherein the AAV capsid variant comprises:
   (a) the amino acid sequence of any of the sequences provided in Tables 1A, 2A, 2B, 21-23, 25, 26, or 30;
   (b) an amino acid sequence comprising at least 3, 4, 5, 6, 7, 8, 9, 10, 11, 12, 13, 14, 15, 16, or 17 consecutive amino acids from any one of the sequences provided in Tables 1A, 2A, 2B, 21-23, 25, 26, or 30;
   (c) an amino acid sequence comprising at least one, two, or three but no more than four different amino acids, relative to any one of the sequences provided in Tables 1A, 2A, 2B, 21-23, 25, 26, or 30; or
   (d) an amino acid sequence comprising at least one, two, or three but no more than four modifications, e.g., substitutions (e.g., conservative substitutions), relative to the amino acid sequence of any one of the sequences provided in Tables 1A, 2A, 2B, 21-23, 25, 26, or 30.
264. An AAV particle comprising an AAV capsid variant and a nucleic acid encoding a SOD1 targeting polynucleotide (e.g., a SOD1 targeting RNA agent, e.g., a siRNA), wherein the AAV capsid variant comprises:
   (a) the amino acid sequence of any of SEQ ID NOs: 945-980 or 985-986;
   (b) an amino acid sequence comprising at least 3, 4, or 5 consecutive amino acids from any one of SEQ ID NOs: 945-980 or 985-986;
   (c) an amino acid sequence comprising at least one, two, or three but no more than four different amino acids, relative to the amino acid sequence of any one of SEQ ID NOs: 945-980 or 985-986; or
   (d) an amino sequence comprising at least one, two, or three but no more than four modifications, e.g., substitutions (e.g., conservative substitutions), relative to the amino acid sequence of any one of SEQ ID NOs: 945-980 or 985-986.
265. An AAV particle comprising an AAV capsid variant and a nucleic acid encoding a SOD1 targeting polynucleotide (e.g., a SOD1 targeting RNA agent, e.g., a siRNA), wherein the AAV capsid variant comprises:
   (a) the amino acid sequence of any of SEQ ID NOs: 2, 200, 201, 941, 943, 204, 208, 404, or 903-909;
   (b) an amino acid sequence comprising at least 3, 4, 5, 6, 7, 8, 9, 10, 11, 12, or 13 consecutive amino acids from any one of SEQ ID NOs: 2, 200, 201, 941, 943, 204, 208, 404, or 903-909;
   (c) an amino acid sequence comprising at least one, two, or three but no more than four different amino acids, relative to the amino acid sequence of any one of SEQ ID NOs: 2, 200, 201, 941, 943, 204, 208, 404, or 903-909; or
   (d) an amino acid sequence comprising at least one, two, or three but no more than four modifications, e.g., substitutions (e.g., conservative substitutions), relative to the amino acid sequence of any one of SEQ ID NOs: 2, 200, 201, 941, 943, 204, 208, 404, or 903-909.
266. The AAV particle of any one of embodiments 262, 263 or 265, wherein the AAV capsid variant comprises an amino acid sequence comprising at least 4, 5, 6, 7, 8, 9, 10, 11, 12, or 13 consecutive amino acids from any one of SEQ ID NOs: 2, 200, 201, 941, 943, 204, 208, 404, or 903-909.
267. The AAV particle of any one of embodiments 262-266, wherein the at least 3 consecutive amino acids comprise SPH.
268. The AAV particle of any one of embodiments 262-267, wherein the at least 4 consecutive amino acids comprise SPHS (SEQ ID NO: 1371).
269. The AAV particle of any one of embodiments 262-268, wherein the at least 5 consecutive amino acids comprise SPHSK (SEQ ID NO: 1372).
270. The AAV particle of any one of embodiments 262-269, wherein the at least 6 consecutive amino acids comprise SPHSKA (SEQ ID NO: 941).
271. The AAV particle of embodiment 262-266, wherein the at least 3 consecutive amino acids comprise HDS.
272. The AAV particle of any one of embodiments 262-266 or 271, wherein at least the 4 consecutive amino acids comprise HDSP (SEQ ID NO: 1373).
273. The AAV particle of any one of embodiments 262-266, 271, or 272, wherein the at least 5 consecutive amino acids comprise HDSPH (SEQ ID NO: 1374).
274. The AAV particle of any one of embodiments 262-266 or 271-273, wherein the at least 6 consecutive amino acids comprise HDSPHK (SEQ ID NO: 2).
275. The AAV particle of any one of embodiments 262-267, wherein:
   (i) the at least 3 consecutive amino acids comprise SPH;
   (ii) the at least 4 consecutive amino acids comprise SPHK (SEQ ID NO: 4816);
   (iii) the at least 5 consecutive amino acids comprise SPHKY (SEQ ID NO: 1386); and/or
   (iv) the at least 6 consecutive amino acids comprise SPHKYG (SEQ ID NO: 966).
276. The AAV particle of embodiment 262, 263, 265, or 266, wherein the AAV capsid variant comprises an amino acid sequence comprising at least one, two, or three but no more than four modifications, e.g., substitutions (e.g., conservative substitutions), relative to the amino acid sequence of any one of SEQ ID NOs: 2, 200, 201, 941, 943, 204, 208, 404, or 903-909.
277. The AAV particle of any one of embodiments 262-270 or 276, wherein the AAV capsid variant comprises an amino acid sequence comprising at least one, two, or three but no more than four modifications, e.g., substitutions (e.g., conservative substitutions), relative to the amino acid sequence of SPHSKA (SEQ ID NO: 941).
278. The AAV particle of any one of embodiments 262-266, 271-274, or 276, wherein the AAV capsid variant comprises an amino acid sequence comprising at least one, two, or three but no more than four modifications, e.g., substitutions (e.g., conservative substitutions), relative to the amino acid sequence of HDSPHK (SEQ ID NO: 2).
279. The AAV particle of any one of embodiments 262-267, 275, or 276, wherein the AAV capsid variant comprises an amino acid sequence comprising at least one, two, or three but no more than four modifications, e.g., substitutions (e.g., conservative substitutions), relative to the amino acid sequence of SPHKYG (SEQ ID NO: 966).
280. The AAV particle of embodiment 262 or 263, wherein the AAV capsid variant comprises:
   (i) an amino acid sequence comprising at least one, two, or three but no more than four modifications, e.g., substitutions (e.g., conservative substitutions), relative to the amino acid sequence of KTENVSGSPHSKAQNQQT (SEQ ID NO: 3272);
   (ii) an amino acid sequence comprising at least one, two, or three but no more than four modifications, e.g., substitutions (e.g., conservative substitutions), relative to the amino acid sequence of KTERVSGSPHSKAQNQQT (SEQ ID NO: 3589);
   (iii) an amino acid sequence comprising at least one, two, or three but no more than four modifications, e.g., substitutions (e.g., conservative substitutions), relative to the amino acid sequence of KAEIGHDSPHKSGQNQQT (SEQ ID NO: 1754);
   (iv) an amino acid sequence comprising at least one, two, or three but no more than four modifications, e.g., substitutions (e.g., conservative substitutions), relative to the amino acid sequence of KTEKMSGSPHSKAQNQQT (SEQ ID NO: 3241);
   (v) an amino acid sequence comprising at least one, two, or three but no more than four modifications, e.g., substitutions (e.g., conservative substitutions), relative to the amino acid sequence of KTINGHDSPHSKAQNLQT (SEQ ID NO: 4100); or
   (vi) an amino acid sequence comprising at least one, two, or three but no more than four modifications, e.g., substitutions (e.g., conservative substitutions), relative to the amino acid sequence of KTVNGHDSPHSKAQNQQT (SEQ ID NO: 4062).
281. The AAV particle of any one of embodiments 262, 263, 265, 266, or 276, wherein the AAV capsid variant comprises an amino acid sequence comprising at least one, two, or three but no more than four different amino acids relative to the amino acid sequence of any one of SEQ ID NOs: 200, 201, 941, 943, 204, 208, 404, or 903-909.
282. The AAV particle of any one of embodiments 262-270, 276, 277, or 281, wherein the AAV capsid variant comprises an amino acid sequence comprising at least one, two, or three but no more than four different amino acids relative to the amino acid sequence of SPHSKA (SEQ ID NO: 941).
283. The AAV particle of any one of embodiments 262-266, 271-275, 276, 278, or 281, wherein the AAV capsid variant comprises an amino acid sequence comprising at least one, two, or three but no more than four different amino acids relative to the amino acid sequence of HDSPHK (SEQ ID NO: 2).
284. The AAV particle of any one of embodiments 262-267, 275, 276, or 281, wherein the AAV capsid variant comprises an amino acid sequence comprising at least one, two, or three but no more than four different amino acids relative to the amino acid sequence of SPHKYG (SEQ ID NO: 966).
285. The AAV particle of any one of embodiments 262, 263 or 280, wherein the AAV capsid variant comprises:
   (i) an amino acid sequence comprising at least one, two, or three but no more than four different amino acids relative to the amino acid sequence of KTERVSGSPHSKAQNQQT (SEQ ID NO: 3589);
   (ii) an amino acid sequence comprising at least one, two, or three but no more than four different amino acids relative to the amino acid sequence of KAEIGHDSPHKSGQNQQT (SEQ ID NO: 1754);
   (iii) an amino acid sequence comprising at least one, two, or three but no more than four different amino acids relative to the amino acid sequence of KTEKMSGSPHSKAQNQQT (SEQ ID NO: 3241);
   (iv) an amino acid sequence comprising at least one, two, or three but no more than four different amino acids relative to the amino acid sequence of KTINGHDSPHSKAQNLQT (SEQ ID NO: 4100);
   (v) an amino acid sequence comprising at least one, two, or three but no more than four different amino acids relative to the amino acid sequence of KTVNGHDSPHSKAQNQQT (SEQ ID NO: 4062);
   (vi) an amino acid sequence comprising at least one, two, or three but no more than four different amino acids relative to the amino acid sequence of KTENVSGSPHSKAQNQQT (SEQ ID NO: 3272);
   (vii) an amino acid sequence comprising at least one, two, or three but no more than four different amino acids relative to the amino acid sequence of KTEKVSGSPHSKAQNQQT (SEQ ID NO: 3274);
   (viii) an amino acid sequence comprising at least one, two, or three but no more than four different amino acids relative to the amino acid sequence of KTINGSGSPHKSGQNKTS (SEQ ID NO: 4406);
   (ix) an amino acid sequence comprising at least one, two, or three but no more than four different amino acids relative to the amino acid sequence of KTSNASGSPHSKAHNQQT (SEQ ID NO: 3382);
   (x) an amino acid sequence comprising at least one, two, or three but no more than four different amino acids relative to the amino acid sequence of KNSIGSGSPHSKAQNQQT (SEQ ID NO: 3421);
   (xi) an amino acid sequence comprising at least one, two, or three but no more than four different amino acids relative to the amino acid sequence of KTVNGHDSPHKSGQRPST (SEQ ID NO: 4478);
   (xii) an amino acid sequence comprising at least one, two, or three but no more than four different amino acids relative to the amino acid sequence of KAENGSGSPHSKAQNQQT (SEQ ID NO: 3487); or
   (xiii) an amino acid sequence comprising at least one, two, or three but no more than four different amino acids relative to the amino acid sequence of KTINGSGSPHKSGQNQQP (SEQ ID NO: 4081).
286. The AAV particle of any one of embodiments 1-129, 262, 263, 265-279, or 281-284, wherein the AAV capsid variant comprises the amino acid sequence of any of SEQ ID NOs: 200, 201, 941, 943, 204, 208, 404, or 903-909.
287. The AAV particle of any one of embodiments 262, 263, 267-270, 276, 277, 280-282, 285, or 286, wherein the AAV capsid variant comprises the amino acid sequence of ERVSGSPHSKA (SEQ ID NO: 4817), optionally wherein the amino acid sequence is present immediately subsequent to position 450 and replaces positions 451-455, numbered according to SEQ ID NO: 138.
288. The AAV particle of any one of embodiments 262, 263, 267-270, 276, 277, 280-282, or 285-287, wherein the AAV capsid variant comprises the amino acid sequence of KTERVSGSPHSKAQNQQT (SEQ ID NO: 3589), optionally wherein the amino acid sequence is present immediately subsequent to position 448 and replaces positions 449-460, numbered according to SEQ ID NO: 138.
289. The AAV particle of any one of embodiments 262, 263, 271-274, 276, 278, 280, 281, 283, 285, or 286, wherein the AAV capsid variant comprises the amino acid sequence of AEIGHDSPHKSG (SEQ ID NO: 4818), optionally wherein the amino acid sequence is present immediately subsequent to position 449 and replaces positions 450-455, numbered according to SEQ ID NO: 138.
290. The AAV particle of any one of embodiments 262, 263, 271-274, 276, 278, 280, 281, 283, 285, 286, or 289, wherein the AAV capsid variant comprises the amino acid sequence of KAEIGHDSPHKSGQNQQT (SEQ ID NO: 1754), optionally wherein the amino acid sequence is present immediately subsequent to position 448 and replaces positions 449-460, (e.g., K449, T450, I451, N452, G453, S454, G455, Q456, N457, Q458, Q459, T460), numbered according to SEQ ID NO: 138.
291. The AAV particle of any one of embodiments 262, 263, 271-274, 276, 278, 280, 281, 283, 285, or 286, which comprises the amino acid sequence of EKMSGSPHSKA (SEQ ID NO: 6401), optionally wherein the amino acid sequence is present immediately subsequent to position 450 and replaces positions 451-455 (e.g., 1451, N452, G453, S454, G455), numbered according to SEQ ID NO: 138.
292. The AAV particle of any one of embodiments 262, 263, 271-274, 276, 278, 280, 281, 283, 285, 286, or 291, wherein the AAV capsid variant comprises the amino acid sequence of KTEKMSGSPHSKAQNQQT (SEQ ID NO: 3241), optionally wherein the amino acid sequence is present immediately subsequent to position 448 and replaces positions 449-460 (e.g., K449, T450, 1451, N452, G453, S454, G455, Q456, N457, Q458, Q459, T460), numbered according to SEQ ID NO: 138.
293. The AAV particle of any one of embodiments 262, 263, 271-274, 276, 278, 280, 281, 283, 285, or 286, wherein the AAV capsid variant comprises the amino acid sequence of HDSPHSKAQNL (SEQ ID NO: 6402), optionally wherein the amino acid sequence is present immediately subsequent to position 453 and replaces positions 456-458 (e.g., Q456, N457, Q458), numbered according to SEQ ID NO: 138.
294. The AAV particle of any one of embodiments 262, 263, 271-274, 276, 278, 280, 281, 283, 285, 286, or 293, wherein the AAV capsid variant comprises the amino acid sequence of KTINGHDSPHSKAQNLQT (SEQ ID NO: 4100), optionally wherein the amino acid sequence is present immediately subsequent to position 448 and replaces positions 449-460 (e.g., K449, T450, 1451, N452, G453, S454, G455, Q456, N457, Q458, Q459, T460), numbered according to SEQ ID NO: 138.
295. The AAV particle of any one of embodiments 262, 263, 271-274, 276, 278, 280, 281, 283, 285, or 286, wherein the AAV capsid variant comprises the amino acid sequence of VNGHDSPHSKA (SEQ ID NO: 6403), optionally wherein the amino acid sequence is present immediately subsequent to position 450 and replaces positions 451-455 (e.g., 1451, N452, G453, S454, G455), numbered according to SEQ ID NO: 138.
296. The AAV particle of any one of embodiments 262, 263, 271-274, 276, 278, 280, 281, 283, 285, 286, or 295, wherein the AAV capsid variant comprises the amino acid sequence of KTVNGHDSPHSKAQNQQT (SEQ ID NO: 4062), optionally wherein the amino acid sequence is present immediately subsequent to position 448 and replaces positions 449-460 (e.g., K449, T450, 1451, N452, G453, S454, G455, Q456, N457, Q458, Q459, T460), numbered according to SEQ ID NO: 138.
297. The AAV particle of any one of embodiments 262, 263, 271-274, 276, 278, 280, 281, 283, 285, or 286, wherein the AAV capsid variant comprises the amino acid sequence of ENVSGSPHSKA (SEQ ID NO: 4819), optionally wherein the amino acid sequence is present immediately subsequent to position 450 and replaces positions 451-455 (e.g., 1451, N452, G453, S454, G455), numbered according to SEQ ID NO: 138; corresponds to positions 451 to 461 of SEQ ID NO: 69; or is present at positions 451 to 461 numbered according to SEQ ID NO: 69 or 981.
298. The AAV particle of any one of embodiments 262, 263, 271-274, 276, 278, 280, 281, 283, 285, 286, or 295, wherein the AAV capsid variant comprises the amino acid sequence of KTENVSGSPHSKAQNQQT (SEQ ID NO: 3272), optionally wherein the amino acid sequence is present immediately subsequent to position 448 and replaces positions 449-460 (e.g., K449, T450, 1451, N452, G453, S454, G455, Q456, N457, Q458, Q459, T460), numbered according to SEQ ID NO: 138; corresponds to positions 449 to 466 of SEQ ID NO: 69; or is present at positions 449 to 466 numbered according to SEQ ID NO: 69 or 981.
299. The AAV particle of any one of embodiments 1-23, 26-29, 32, 35-43, 46-51, 54-71, 79-89, 94-99, 102-104, 106-112, 115-164, 203-209, 212, 216-224, 234-243, 248-250, 253-270, 276, 277, 281, 282, or 286-288, wherein the AAV capsid variant comprises an amino acid sequence encoded by: the nucleotide sequence of SEQ ID NO: 942; a nucleotide sequence comprising at least one, two, three, four, five, six, or seven modifications, e.g., substitutions, but no more than ten modifications, e.g., substitutions, relative to the nucleotide sequence of SEQ ID NO: 942; or a nucleotide sequence comprising at least one, two, three, four, five, six, or seven, but no more than ten different nucleotides relative to the nucleotide sequence of SEQ ID NO: 942.
300. The AAV particle of any one of embodiments 1-9, 11, 12-22, 24, 26, 28, 30, 33, 35-42, 44, 46-50, 52, 54-78, 83-88, 90-98, 100-103, 105-111, 113-129, 165-211, 213-222, 226-242, 245-249, 251-266, 271-274, 276, 278, 280, 281, 283, 285, 286, 289, or 290, wherein the AAV capsid variant comprises an amino acid sequence encoded by: the nucleotide sequence of SEQ ID NO: 3; a nucleotide sequence comprising at least one, two, three, four, five, six, or seven modifications, e.g., substitutions, but no more than ten modifications, e.g., substitutions, relative to the nucleotide sequence of SEQ ID NO: 3; or a nucleotide sequence comprising at least one, two, three, four, five, six, or seven, but no more than ten different nucleotides relative to the nucleotide sequence of SEQ ID NO: 3.
301. The AAV particle of any one of embodiments 1-23, 26-29, 32, 35-43, 46-51, 54-71, 79-89, 94-99, 102-104, 106-112, 115-164, 203-209, 212, 216-224, 234-243, 248-250, 253-270, 276, 277, 281, 282, 286-288, or 299, wherein the nucleotide sequence encoding the AAV capsid variant comprises the nucleotide sequence of SEQ ID NO: 942; a nucleotide sequence comprising at least one, two, three, four, five, six, or seven modifications, e.g., substitutions, but no more than ten modifications, e.g., substitutions, relative to the nucleotide sequence of SEQ ID NO: 942; or a nucleotide sequence comprising at least one, two, three, four, five, six, or seven, but no more than ten different nucleotides relative to the nucleotide sequence of SEQ ID NO: 942.
302. The AAV particle of any one of embodiments 1-9, 11, 12-22, 24, 26, 28, 30, 33, 35-42, 44, 46-50, 52, 54-78, 83-88, 90-98, 100-103, 105-111, 113-129, 165-211, 213-222, 226-242, 245-249, 251-266, 271-274, 276, 278, 280, 281, 283, 285, 286, 289, 290, or 300, wherein the nucleotide sequence encoding the AAV capsid variant comprises the nucleotide sequence of SEQ ID NO: 3; a nucleotide sequence comprising at least one, two, three, four, five, six, or seven modifications, e.g., substitutions, but no more than ten modifications, e.g., substitutions, relative to the nucleotide sequence of SEQ ID NO: 3; or a nucleotide sequence comprising at least one, two, three, four, five, six, or seven, but no more than ten different nucleotides relative to the nucleotide sequence of SEQ ID NO: 3.
303. The AAV particle of any one of embodiments 262-302, wherein the amino acid sequence is present in loop IV of the AAV capsid variant.
304. The AAV particle of any one of embodiments 262-303, wherein the amino acid sequence is present immediately subsequent to position 448, 449, 450, 451, 452, 453, 454, or 455, relative to a reference sequence numbered according to the amino acid sequence of SEQ ID NO: 138.
305. The AAV particle of any one of embodiments 262-304, wherein the amino acid sequence replaces amino acids 449, 450, 451, 452, 453, 454, 455, 456, 457, 458, 459, and/or 460 (e.g., K449, T450, I451, N452, G453, S454, G455, Q456, N457, Q458, Q459, and/or T460), relative to a reference sequence numbered according to the amino acid sequence of SEQ ID NO: 138.
306. The AAV particle of any one of embodiments 262-305, wherein the amino acid sequence is present immediately subsequent to position 455, relative to a reference sequence numbered according to the amino acid sequence of SEQ ID NO: 138.
307. The AAV particle of any one of embodiments 262-306, wherein the amino acid sequence is present immediately subsequent to position 453, relative to a reference sequence numbered according to the amino acid sequence of SEQ ID NO: 138.
308. The AAV particle of any one of embodiments 1-23, 26-29, 32, 35-43, 46-51, 54-71, 79-89, 94-99, 102-104, 106-112, 115-164, 203-209, 212, 216-224, 234-243, 248-250, 253-270, 276, 277, 281, 282, 286-288, 299, 301, or 303-307, wherein the AAV capsid variant comprises the amino acid sequence of SPHSKA (SEQ ID NO: 941), wherein the amino acid sequence is present immediately subsequent to position 455, relative to a reference sequence numbered according to the amino acid sequence of SEQ ID NO: 138.
309. The AAV particle of any one of embodiments 1-23, 26-29, 32, 35-43, 46-51, 54-71, 79-89, 94-99, 102-104, 106-112, 115-164, 203-209, 212, 216-224, 234-243, 248-250, 253-270, 276, 277, 281, 282, 286-288, 299, 301, or 303-308, wherein the AAV capsid variant comprises the amino acid sequence of SPHSKA (SEQ ID NO: 941), wherein the amino acid sequence is present immediately subsequent to position 455 (e.g., present at positions 456-461), numbered according to the amino acid sequence of SEQ ID NO: 981.
310. The AAV particle of embodiment 308 or 309, wherein the AAV capsid variant further comprises an amino acid other than I at position 451, an amino acid other than N at position 452, and an amino acid other than G at position 453, numbered according to any one of SEQ ID NOs: 36, 138, or 981.
311. The AAV particle of any one of embodiments 308-310, wherein the AAV capsid variant further comprises E at position 451, R at position 452, and V at position 453, numbered according to any one of SEQ ID NOs: 36, 138, or 981.
312. The AAV particle of any one of embodiments 308-311, wherein the AAV capsid variant further comprises the substitutions I451E, N452R, and G453V, numbered according to any one of SEQ ID NOs: 36, 138, or 981.
313. The AAV particle of any one of embodiments 308-312, wherein the AAV capsid variant comprises:
   (i) E at position 451, R at position 452, and V at position 453, numbered according to any one of SEQ ID NOs: 36, 138, or 981; and
   (ii) the amino acid sequence of SPHSKA (SEQ ID NO: 941), wherein the amino acid sequence is present immediately subsequent to position 455, numbered according to any one of SEQ ID NOs: 36, 138, or 981 (e.g., at amino acids 456-461, numbered according to SEQ ID NO: 36 or 981).
314. The AAV particle of embodiment 308 or 309, which further comprises an amino acid other than I at position 451, an amino acid other than N at position 452, and/or G at position 453, numbered according to SEQ ID NO: 39 or 138.
315. The AAV particle of any one of embodiments 308, 309, or 314, which further comprises E at position 451, K at position 452, and/or M at position 453, numbered according to SEQ ID NO: 138 or 39.
316. The AAV particle of any one of embodiments 308, 309, 314, or 315, which further comprises the substitutions I451E, N452K, and G453M, numbered according to SEQ ID NO: 39 or 138.
317. The AAV particle of any one of embodiments 308, 309, or 314-316, which comprises:
   (i) E at position 451, K at position 452, and M at position 453, numbered according to SEQ ID NO: 39 or 138; and
   (ii) the amino acid sequence of SPHSKA (SEQ ID NO: 941), wherein the amino acid sequence is present immediately subsequent to position 455, numbered according to SEQ ID NO: 39 or 138.
318. The AAV particle of embodiment 308 or 309, which further comprises an amino acid other than S at position 454, an amino acid other than G at position 455, and/or Q at position 458, numbered according to SEQ ID NO: 138.
319. The AAV particle of any one of embodiments 308, 309, or 318, which further comprises H at position 454, D at position 455, and/or L at position 458, numbered according to SEQ ID NO: 138.
320. The AAV particle of any one of embodiments 308, 309, 318, or 319, which further comprises the substitutions S454H, G455D, and Q458L, numbered according to SEQ ID NO: 138.
321. The AAV particle of any one of embodiments 308, 309, or 318-320, which comprises:
   (i) H at position 454, D at position 455, and/or L at position 458, numbered according to SEQ ID NO: 138; and
   (ii) the amino acid sequence of SPHSKA (SEQ ID NO: 941), wherein the amino acid sequence is present immediately subsequent to position 455, numbered according to SEQ ID NO: 138.
322. The AAV particle of embodiment 308 or 309, which further comprises an amino acid other than I at position 451, an amino acid other than S at position 454, and/or an amino acid other than G at position 455, numbered according to SEQ ID NO: 52 or 138.
323. The AAV particle of any one of embodiments 308, 309, or 322, which further comprises V at position 451, H at position 454, and/or D at position 455, numbered according to SEQ ID NO: 52 or 138.
324. The AAV particle of any one of embodiments 308, 309, 322, or 323, wherein the AAV capsid variant further comprises the substitutions I451V, S454H, and/or G455D, numbered according to SEQ ID NO: 52 or 138.
325. The AAV particle of any one of embodiments 308, 309, or 322-324, wherein the AAV capsid variant comprises:
   (i) V at position 451, H at position 454, and/or D at position 455, numbered according to SEQ ID NO: 52 or 138; and
   (ii) the amino acid sequence of SPHSKA (SEQ ID NO: 941), wherein the amino acid sequence is present immediately subsequent to position 455, numbered according to SEQ ID NO: 52 or 138.
326. The AAV particle of embodiment 308 or 309, wherein the AAV capsid variant comprises an amino acid other than I at position 451 and/or G at position 453, numbered according to SEQ ID NO: 138.
327. The AAV particle of any one of embodiments 308, 309, or 326, wherein the AAV capsid variant comprises E at position 451 and/or V at position 453, numbered according to SEQ ID NO: 138 or 69.
328. The AAV particle of any one of embodiments 308, 309, 326, or 327, wherein the AAV capsid variant comprises:
   (i) E at position 451 and V at position 453, numbered according to SEQ ID NO: 69 or 138; and
   (ii) the amino acid sequence of SPHSKA (SEQ ID NO: 941), wherein the amino acid sequence is present immediately subsequent to position 455, at positions 456-461, numbered according to SEQ ID NO: 69 or 138.
329. The AAV particle of embodiment 308 or 309, wherein the AAV capsid variant comprises:
   (i) E at position 451, K at position 452, and V at position 453, numbered according to SEQ ID NO: 70 or 138; and the amino acid sequence of SPHSKA (SEQ ID NO: 941), wherein the amino acid sequence is present immediately subsequent to position 455, at positions 456-461, numbered according to SEQ ID NO: 70 or 138;
   (ii) S at position 451, A at position 453, and H at position 462, numbered according to SEQ ID NO: 4; and the amino acid sequence of SPHSKA (SEQ ID NO: 941), wherein the amino acid sequence is present immediately subsequent to position 455, at positions 456-461, numbered according to SEQ ID NO: 4 or 138;
   (iii) N at position 450, S at position 451, and I at position 452, numbered according to SEQ ID NO: 5 or 138; and the amino acid sequence of SPHSKA (SEQ ID NO: 941), wherein the amino acid sequence is present immediately subsequent to position 455, at positions 456-461, numbered according to SEQ ID NO: 5 or 138; or
   (iv) A at position 450 and E at position 451, numbered according to SEQ ID NO: 7 or 138; and the amino acid sequence of SPHSKA (SEQ ID NO: 941), wherein the amino acid sequence is present immediately subsequent to position 455, at positions 456-461, numbered according to SEQ ID NO: 7 or 138.
330. The AAV particle of any one of embodiments 308, 309, or 329, wherein the AAV capsid variant comprises the amino acid sequence of any one of SEQ ID NOs: 3241, 3272, 3274, 3382, 3421, 3487, 3589, 4100, optionally wherein:
   (i) the amino acid sequence is present immediately subsequent to position 448 and replaces positions 449-460 (e.g., K449, T450, I451, N452, G453, S454, G455, Q456, N457, Q458, Q459, and T460), numbered according to SEQ ID NO: 138; or
   (ii) the amino acid sequence is present at positions 449-466, numbered according to any one of SEQ ID NOs: 69, 70, 71, 4, 5, 6, 7, 8, 36-59, 981, or 982.
331. The AAV particle of any one of embodiments 1-9, 11, 12-22, 24, 26, 28, 30, 33, 35-42, 44, 46-50, 52, 54-78, 83-88, 90-98, 100-103, 105-111, 113-129, 165-211, 213-222, 226-242, 245-249, 251-266, 271-274, 276, 278, 280, 281, 283, 285, 286, 289, 290, 300, or 302-307, wherein the AAV capsid variant comprises the amino acid sequence of HDSPHK (SEQ ID NO: 2), wherein the amino acid sequence is present immediately subsequent to position 453 (e.g., at positions 454-459), numbered relative to a reference sequence numbered according to the amino acid sequence of SEQ ID NO: 138.
332. The AAV particle of any one of embodiments 1-9, 11, 12-22, 24, 26, 28, 30, 33, 35-42, 44, 46-50, 52, 54-78, 83-88, 90-98, 100-103, 105-111, 113-129, 165-211, 213-222, 226-242, 245-249, 251-266, 271-274, 276, 278, 280, 281, 283, 285, 286, 289, 290, 300, 302-307, or 331, wherein the AAV capsid variant comprises the amino acid sequence of HDSPHK (SEQ ID NO: 2), wherein the amino acid sequence is present immediately subsequent to position 453, numbered relative to a reference sequence numbered according to the amino acid sequence of SEQ ID NO: 982.
333. The AAV particle of embodiment 332, wherein the AAV capsid variant comprises:
   (i) V at position 451, R at position 463, P at position 464, and S at position 465, numbered according to SEQ ID NO: 6 or 982;
   (ii) the amino acid sequence of HDSPHK (SEQ ID NO: 2), wherein the amino acid sequence is present immediately subsequent to position 453, at positions 454-459, numbered relative to a reference sequence numbered according to the amino acid sequence of SEQ ID NO: 6 or 982.
334. The AAV particle of embodiment 332 or 333, wherein the AAV capsid variant comprises the amino acid sequence of SEQ ID NO: 1754, 4062, 4100, or 4478, optionally wherein:
   (i) the amino acid sequence is present immediately subsequent to position 448 and replaces positions 449-460 (e.g., K449, T450, 1451, N452, G453, S454, G455, Q456, N457, Q458, Q459, and T460), numbered according to SEQ ID NO: 138; or
   (ii) the amino acid sequence is present at positions 449-466, numbered according to any one of SEQ ID NOs: 69, 70, 71, 4, 5, 6, 7, 8, 36-59, 981 or 982.
335. The AAV particle of any one of embodiments 1-9, 11, 12-22, 24, 26, 28, 30, 33, 35-42, 44, 46-50, 52, 54-78, 83-88, 90-98, 100-103, 105-111, 113-129, 165-211, 213-222, 226-242, 245-249, 251-266, 271-274, 276, 278, 280, 281, 283, 285, 286, 289, 290, 300, 302-307, 331, or 332, wherein the AAV capsid variant comprises the amino acid sequence of SPHKSG (SEQ ID NO: 946), wherein the amino acid sequence is present immediately subsequent to position 455 (e.g., at positions 456-461), numbered relative to a reference sequence numbered according to the amino acid sequence of SEQ ID NO: 982.
336. The AAV particle of embodiment 335, wherein the AAV capsid variant comprises:
   (i) K at position 464, T at position 465, and S at position 466, numbered according SEQ ID NO: 71; and the amino acid sequence of SPHKSG (SEQ ID NO: 946), wherein the amino acid sequence is present immediately subsequent to position 455, at positions 456-461, numbered relative to a reference sequence numbered according to the amino acid sequence of SEQ ID NO: 71; or
   (ii) P at position 466, numbered according SEQ ID NO: 8; and the amino acid sequence of SPHKSG (SEQ ID NO: 946), wherein the amino acid sequence is present immediately subsequent to position 455, at positions 456-461, numbered relative to a reference sequence numbered according to the amino acid sequence of SEQ ID NO: 8.
337. The AAV particle of embodiment 335 or 336, wherein the AAV capsid variant comprises the amino acid sequence of SEQ ID NO: 4081 or 4406, optionally wherein:
   (i) the amino acid sequence is present immediately subsequent to position 448 and replaces positions 449-460 (e.g., K449, T450, I451, N452, G453, S454, G455, Q456, N457, Q458, Q459, and T460), numbered according to SEQ ID NO: 138; or
   (ii) the amino acid sequence is present at positions 449-466, numbered according to any one of SEQ ID NOs: 69, 70, 71, 4, 5, 6, 7, 8, 36-59, 981 or 982.
338. The AAV particle of any one of embodiments 1-9, 11, 12-22, 24, 26, 28, 30, 33, 35-42, 44, 46-50, 52, 54-78, 83-88, 90-98, 100-103, 105-111, 113-129, 165-211, 213-222, 226-242, 245-249, 251-266, 271-274, 276, 278, 280, 281, 283, 285, 286, 289, 290, 300, 302-307, or 331-337, wherein the AAV capsid variant comprises:
   (i) the amino acid sequence of HDSPHSKA (SEQ ID NO: 4486), which is present immediately subsequent to position 453; and
   (ii) a deletion of amino acids SG at position 454 and 455;
   wherein (i) and (ii) are numbered according to SEQ ID NO: 138.
339. The AAV particle of any one of embodiments 1-9, 11, 12-22, 24, 26, 28, 30, 33, 35-42, 44, 46-50, 52, 54-78, 83-88, 90-98, 100-103, 105-111, 113-129, 165-211, 213-222, 226-242, 245-249, 251-266, 271-274, 276, 278, 280, 281, 283, 285, 286, 289, 290, 300, 302-307, or 331-338, wherein the AAV capsid variant comprises the amino acids HD at position 454 and 455, and further comprises the amino acid sequence of SPHSKA (SEQ ID NO: 941), which is present immediately subsequent to position 455, numbered relative to SEQ ID NO: 138.
340. The AAV particle of any one of embodiments 331-339, wherein the AAV capsid variant further comprises an amino acid other than T at position 450, an amino acid other than I at position 451, and an amino acid other than N at position 452, numbered according to SEQ ID NO: 138 or 982.
341. The AAV particle of any one of embodiments 331-340, wherein the AAV capsid variant further comprises A at position 450, E at position 451, and I at position 452, numbered according to SEQ ID NO: 138 or 982.
342. The AAV particle of any one of embodiments 331-341, wherein the AAV capsid variant further comprises the substitutions T450A, I451E, and N452I, numbered according to SEQ ID NO: 138 or 982.
343. The AAV particle of any one of embodiments 331-342, wherein the AAV capsid variant comprises:
   (i) A at position 450, E at position 451, and I at position 452, numbered according to SEQ ID NO: 138 or 982; and
   (ii) the amino acid sequence of HDSPHK (SEQ ID NO: 2), which is present immediately subsequent to positions 453, numbered according to SEQ ID NO: 138 or 982.
344. The AAV particle of any one of embodiments 1-22, 25-27, 31, 34-42, 45-50, 51-63, 69, 79, 83-86, 94-98, 102, 103, 110, 111, 118-129, 262-267, 275, 276, 281, 284, 286, or 303-307, wherein the AAV capsid variant comprises the amino acid sequence of SPHKYG (SEQ ID NO: 966), wherein the amino acid sequence is present immediately subsequent to position 455, relative to a reference sequence numbered according to the amino acid sequence of SEQ ID NO: 138.
345. An AAV particle comprising an AAV capsid variant and a nucleic acid encoding a SOD1 targeting polynucleotide (e.g., a SOD1 targeting RNA agent, e.g., a siRNA), wherein the AAV capsid variant comprises the amino acid sequence of HDSPHK (SEQ ID NO: 2), wherein the amino acid sequence is present immediately subsequent to position 453, numbered according to the amino acid sequence of SEQ ID NO: 982.
346. An AAV particle comprising an AAV capsid variant and a nucleic acid encoding a SOD1 targeting polynucleotide (e.g., a SOD1 targeting RNA agent, e.g., a siRNA), wherein the AAV capsid variant comprises the amino acid sequence of SPHSKA (SEQ ID NO: 941), wherein the amino acid sequence is present immediately subsequent to position 455, numbered according to the amino acid sequence of SEQ ID NO: 981.
347. An AAV particle comprising an AAV capsid variant and a nucleic acid encoding a SOD1 targeting polynucleotide (e.g., a SOD1 targeting RNA agent, e.g., a siRNA), wherein the AAV capsid variant comprises the amino acid sequence of HDSPHK (SEQ ID NO: 2), wherein the amino acid sequence is present immediately subsequent to position 453, numbered according to the amino acid sequence of SEQ ID NO: 37, and optionally further comprising:
   (i) one, two, or all of an amino acid other than T at position 450, an amino acid other than I at position 541, and/or an amino acid other than N at position 452, numbered according to SEQ ID NO: 138 or 37;
   (ii) one, two, or all of A at position 450, E at position 451, and/or I at position 452, numbered according to SEQ ID NO: 138 or 37.
348. An AAV particle comprising an AAV capsid variant and a nucleic acid encoding a SOD1 targeting polynucleotide (e.g., a SOD1 targeting RNA agent, e.g., a siRNA), wherein the AAV capsid variant comprises the amino acid sequence of SPHSKA (SEQ ID NO: 941), wherein the amino acid sequence is present immediately subsequent to position 455, numbered according to the amino acid sequence of any one of SEQ ID NO: 69, 36, 38-55, 57, or 59.
349. The AAV particle of embodiment 348, which comprises the amino acid E at position 451, numbered according to SEQ ID NO: 69, 981, or 138.
350. The AAV particle of embodiment 348 or 349, which comprises the amino acid V at position 453, numbered according to SEQ ID NO: 69, 981, or 138.
351. The AAV particle of any one of embodiments 348-350, which comprises the amino acid E at position 451 and the amino acid V at position 453, numbered according to SEQ ID NO: 69, 981, or 138.
352. The AAV particle of any one of embodiments 348-351, which comprises the amino acid R at position 452, numbered according to SEQ ID NO: 36, 981, or 138.
353. The AAV particle of any one of embodiments 348-352, which comprises the amino acid E at position 451, the amino acid R at position 452, and the amino acid V at position 453, numbered according to SEQ ID NO: 36, 981, or 138.
354. The AAV particle, of any one of the preceding embodiments, wherein the AAV capsid variant further comprises:
   (i) a modification, e.g., an insertion, substitution (e.g., conservative substitution), and/or deletion, in loop I, II, VI and/or VIII; and/or
   (ii) a substitution at position K449, e.g., a K449R substitution, numbered according to SEQ ID NO: 138.
355. The AAV particle of any one of the preceding embodiments, wherein the AAV capsid variant comprises:
   (i) an amino acid sequence comprising at least one, two or three modifications, e.g., substitutions (e.g., conservative substitutions), but not more than 30, 20 or 10 modifications, e.g., substitutions (e.g., conservative substitutions), relative to the amino acid sequence of SEQ ID NO: 138; or
   (ii) an amino acid sequence comprising at least one, two or three, but no more than 30, 20 or 10 different amino acids relative to the amino acid sequence of SEQ ID NO: 138.
356. The AAV particle of any one of the preceding embodiments, wherein the AAV capsid variant comprises the amino acid sequence of SEQ ID NO: 138, or an amino acid sequence with at least 80% (e.g., at least about 85, 90, 95, 96, 97, 98, or 99%) sequence identity thereto.
357. The AAV particle of any one of the preceding embodiments, wherein the AAV capsid variant comprises the amino acid sequence of SEQ ID NO: 138.
358. The AAV particle of any one of the preceding embodiments, wherein the AAV capsid variant comprises an amino acid sequence encoded by the nucleotide sequence of SEQ ID NO: 137, or a sequence with at least 80% (e.g., at least about 85, 90, 95, 96, 97, 98, or 99%) sequence identity thereto.
359. The AAV particle of any one of the preceding embodiments, wherein the nucleotide sequence encoding the AAV capsid variant comprises the nucleotide sequence of SEQ ID NO: 137, or a sequence with at least 80% (e.g., at least about 85, 90, 95, 96, 97, 98, or 99%) sequence identity thereto.
360. The AAV particle of any one of the preceding embodiments, wherein the AAV capsid variant comprises a VP1 protein, a VP2 protein, a VP3 protein, or a combination thereof.
361. The AAV particle of any one of embodiments 1-360, wherein the AAV capsid variant comprises the amino acid sequence corresponding to positions 138-742, e.g., a VP2, of SEQ ID NO: 981, 982, 69, 70, 71, 4, 5, 6, 7, 8, or 36-59, or a sequence with at least 80% (e.g., at least about 85, 90, 95, 96, 97, 98, or 99%) sequence identity thereto.
362. The AAV particle of any one of embodiments 1-360, wherein the AAV capsid variant comprises the amino acid sequence corresponding to positions 203-742, e.g., a VP3, of SEQ ID NO: 981, 982, 69, 70, 71, 4, 5, 6, 7, 8, or 36-59, or a sequence with at least 80% (e.g., at least about 85, 90, 95, 96, 97, 98, or 99%) sequence identity thereto.
363. The AAV particle of any one of embodiments 1-360, wherein the AAV capsid variant comprises the amino acid sequence corresponding to positions 138-736, e.g., a VP2, of SEQ ID NO: 138, or a sequence with at least 80% (e.g., at least about 85, 90, 95, 96, 97, 98, or 99%) sequence identity thereto.
364. The AAV particle of any one of embodiments 1-360, wherein the AAV capsid variant comprises the amino acid sequence corresponding to positions 203-736, e.g., a VP3, of SEQ ID NO: 138, or a sequence with at least 80% (e.g., at least about 85, 90, 95, 96, 97, 98, or 99%) sequence identity thereto.
365. The AAV particle of any one of embodiments 1-23, 26-29, 32, 35-43, 46-51, 54-71, 79-89, 94-99, 102-104, 106-112, 115-164, 203-209, 212, 216-224, 234-243, 248-250, 253-270, 276, 277, 281, 282, 286-288, 299, 301, 303-309, or 354, wherein the AAV capsid variant comprises an amino acid sequence comprising at least 3, 4, 5, or 6 consecutive amino acids from the amino acid sequence of SPHSKA (SEQ ID NO: 941), wherein:
   (i) the at least 3 consecutive amino acids comprise SPH;
   (ii) the at least 4 consecutive amino acids comprise SPHS (SEQ ID NO: 1371);
   (iii) the at least 5 consecutive amino acids comprise SPHSK (SEQ ID NO: 1372); or
   (iv) the at least 6 consecutive amino acids comprise SPHSKA (SEQ ID NO: 941);
   wherein the AAV capsid variant comprises: (a) a VP1 protein comprising the amino acid sequence of SEQ ID NO: 138 or SEQ ID NO: 981; (b) a VP2 protein comprising the amino acid sequence of positions 138-736 of SEQ ID NO: 138 or positions 138-742 of SEQ ID NO: 981; (c) a VP3 protein comprising the amino acid sequence of positions 203-736 of SEQ ID NO: 138 or positions 203-742 of SEQ ID NO: 981; or (d) an amino acid sequence with at least 90% (e.g., at least about 95, 96, 97, 98, or 99%) sequence identity to any of the amino acid sequences in (a)-(c).
366. The AAV particle of any one of embodiments 1-23, 26-29, 32, 35-43, 46-51, 54-71, 79-89, 94-99, 102-104, 106-112, 115-164, 203-209, 212, 216-224, 234-243, 248-250, 253-270, 276, 277, 281, 282, 286-288, 299, 301, 303-309, 354, or 365, wherein the AAV capsid variant comprises an amino acid sequence comprising at least 3, 4, 5, or 6 consecutive amino acids from the amino acid sequence of SPHSKA (SEQ ID NO: 941), wherein:
   (i) the at least 3 consecutive amino acids comprise SPH;
   (ii) the at least 4 consecutive amino acids comprise SPHS (SEQ ID NO: 1371);
   (iii) the at least 5 consecutive amino acids comprise SPHSK (SEQ ID NO: 1372); or
   (iv) the at least 6 consecutive amino acids comprise SPHSKA (SEQ ID NO: 941);
   wherein the AAV capsid variant comprises an amino acid sequence at least 90% (e.g., at least about 95, 96, 97, 98, or 99%) identical to the amino acid sequence of SEQ ID NO: 981.
367. The AAV particle of any one of embodiments 1-23, 26-29, 32, 35-43, 46-51, 54-71, 79-89, 94-99, 102-104, 106-112, 115-164, 203-209, 212, 216-224, 234-243, 248-250, 253-270, 276, 277, 281, 282, 286-288, 299, 301, 303-309, 354, 365, or 366, wherein the AAV capsid variant comprises one or two, but no more than three substitutions relative to the amino acid sequence of SPHSKA (SEQ ID NO: 941), wherein the AAV capsid variant comprises:
   (a) a VP1 protein comprising the amino acid sequence of SEQ ID NO: 138 or SEQ ID NO: 981;
   (b) a VP2 protein comprising the amino acid sequence of positions 138-736 of SEQ ID NO: 138 or positions 138-742 of SEQ ID NO: 981;
   (c) a VP3 protein comprising the amino acid sequence of positions 203-736 of SEQ ID NO: 138 or positions 203-742 of SEQ ID NO: 981; or
   (d) an amino acid sequence with at least 90% (e.g., at least about 95, 96, 97, 98, or 99%) sequence identity to any of the amino acid sequences in (a)-(c).
368. The AAV particle of any one of embodiments 1-23, 26-29, 32, 35-43, 46-51, 54-71, 79-89, 94-99, 102-104, 106-112, 115-164, 203-209, 212, 216-224, 234-243, 248-250, 253-270, 276, 277, 281, 282, 286-288, 299, 301, 303-309, 354, or 365-367, wherein the AAV capsid variant comprises one or two, but no more than three substitutions relative to the amino acid sequence of SPHSKA (SEQ ID NO: 941), wherein the AAV capsid variant comprises an amino acid sequence at least 90% (e.g., at least about 95, 96, 97, 98, or 99%) identical to the amino acid sequence of SEQ ID NO: 138 or SEQ ID NO: 981.
369. The AAV particle of any one of embodiments 365-368, wherein the amino acid sequence is present immediately subsequent to position 455, numbered according to SEQ ID NO: 138 or 981.
370. The AAV particle of any one of embodiments 1-9, 11, 12-22, 24, 26, 28, 30, 33, 35-42, 44, 46-50, 52, 54-78, 83-88, 90-98, 100-103, 105-111, 113-129, 165-211, 213-222, 226-242, 245-249, 251-266, 271-274, 276, 278, 280, 281, 283, 285, 286, 289, 290, 300, 302-307, 331-339, or 354, wherein the AAV capsid variant comprises an amino acid sequence comprising at least 3, 4, 5, or 6 consecutive amino acids from the amino acid sequence of HDSPHK (SEQ ID NO: 2), wherein:
   (i) the at least 3 consecutive amino acids comprise HDS;
   (ii) the at least 4 consecutive amino acids comprise HDSP (SEQ ID NO: 1373);
   (iii) the at least 5 consecutive amino acids comprise HDSPH (SEQ ID NO: 1374); or
   (iv) the at least 6 consecutive amino acids comprise HDSPHK (SEQ ID NO: 2);
   wherein the AAV capsid variant comprises: (a) a VP1 protein comprising the amino acid sequence of SEQ ID NO: 138 or SEQ ID NO: 982; (b) a VP2 protein comprising the amino acid sequence of positions 138-736 of SEQ ID NO: 138 or positions 138-742 of SEQ ID NO: 982; (c) a VP3 protein comprising the amino acid sequence of positions 203-736 of SEQ ID NO: 138 or positions 203-742 of SEQ ID NO: 982; or (d) an amino acid sequence with at least 90% (e.g., at least about 95, 96, 97, 98, or 99%) sequence identity to any of the amino acid sequences in (a)-(c).
371. The AAV particle of any one of embodiments 1-9, 11, 12-22, 24, 26, 28, 30, 33, 35-42, 44, 46-50, 52, 54-78, 83-88, 90-98, 100-103, 105-111, 113-129, 165-211, 213-222, 226-242, 245-249, 251-266, 271-274, 276, 278, 280, 281, 283, 285, 286, 289, 290, 300, 302-307, 331-339, 354, or 370, wherein the AAV capsid variant comprises an amino acid sequence comprising at least 3, 4, 5, or 6 consecutive amino acids from the amino acid sequence of HDSPHK (SEQ ID NO: 2), wherein:
   (i) the at least 3 consecutive amino acids comprise HDS;
   (ii) the at least 4 consecutive amino acids comprise HDSP (SEQ ID NO: 1373);
   (iii) the at least 5 consecutive amino acids comprise HDSPH (SEQ ID NO: 1374); or
   (iv) the at least 6 consecutive amino acids comprise HDSPHK (SEQ ID NO: 2);
   wherein the AAV capsid variant comprises an amino acid sequence at least 90% (e.g., at least about 95, 96, 97, 98, or 99%) identical to the amino acid sequence of SEQ ID NO: 982.
372. The AAV particle of any one of embodiments 1-9, 11, 12-22, 24, 26, 28, 30, 33, 35-42, 44, 46-50, 52, 54-78, 83-88, 90-98, 100-103, 105-111, 113-129, 165-211, 213-222, 226-242, 245-249, 251-266, 271-274, 276, 278, 280, 281, 283, 285, 286, 289, 290, 300, 302-307, 331-339, 354, 370, or 371, wherein the AAV capsid variant comprises one or two, but no more than three substitutions relative to the amino acid sequence of HDSPHK (SEQ ID NO: 2), wherein the AAV capsid variant comprises:
   (a) a VP1 protein comprising the amino acid sequence of SEQ ID NO: 138 or SEQ ID NO: 982;
   (b) a VP2 protein comprising the amino acid sequence of positions 138-736 of SEQ ID NO: 138 or positions 138-742 of SEQ ID NO: 982;
   (c) a VP3 protein comprising the amino acid sequence of positions 203-736 of SEQ ID NO: 138 or positions 203-742 of SEQ ID NO: 982; or
   (d) an amino acid sequence with at least 90% (e.g., at least about 95, 96, 97, 98, or 99%) sequence identity to any of the amino acid sequences in (a)-(c).
373. The AAV particle of any one of embodiments 1-9, 11, 12-22, 24, 26, 28, 30, 33, 35-42, 44, 46-50, 52, 54-78, 83-88, 90-98, 100-103, 105-111, 113-129, 165-211, 213-222, 226-242, 245-249, 251-266, 271-274, 276, 278, 280, 281, 283, 285, 286, 289, 290, 300, 302-307, 331-339, 354, 370-372, wherein the AAV capsid variant comprises one or two, but no more than three substitutions relative to the amino acid sequence of HDSPHK (SEQ ID NO: 2), wherein the AAV capsid variant comprises an amino acid sequence at least 90% (e.g., at least about 95, 96, 97, 98, or 99%) identical to the amino acid sequence of SEQ ID NO: 982.
374. The AAV particle of any one of embodiments 370-373, wherein the amino acid sequence is present immediately subsequent to position 453, numbered according to SEQ ID NO: 138 or 982.
375. The AAV particle of any one of embodiments 1-373, wherein the AAV capsid variant comprises the amino acid sequence of any one of SEQ ID NO: 981 or 982, or an amino acid sequence with at least 80% (e.g., at least about 85, 90, 95, 96, 97, 98, or 99%) sequence identity thereto.
376. The AAV particle of any one of embodiments 1-375, wherein the AAV capsid variant comprises an amino acid sequence comprising at least one, two or three modifications, e.g., substitutions (e.g., conservative substitutions), but not more than 30, 20 or 10 modifications, e.g., substitutions (e.g., conservative substitutions), relative to the amino acid sequence of SEQ ID NO: 981 or 982.
377. The AAV particle of any one of embodiments, 1-376, wherein the AAV capsid variant comprises an amino acid sequence comprising at least one, two or three, but not more than 30, 20 or 10 different amino acids, relative to the amino acid sequence of SEQ ID NO: 981 or 982.
378. The AAV particle of any one of embodiments 1-23, 26-29, 32, 35-43, 46-51, 54-71, 79-89, 94-99, 102-104, 106-112, 115-164, 203-209, 212, 216-224, 234-243, 248-250, 253-270, 276, 277, 281, 282, 286-288, 299, 301, 303-309, 354, 365-368, or 375-377, wherein the AAV capsid variant comprises the amino acid sequence of SEQ ID NO: 981, or an amino acid sequence with at least 80% (e.g., at least about 85, 90, 95, 96, 97, 98, or 99%) sequence identity thereto.
379. The AAV particle of any one of embodiments 1-23, 26-29, 32, 35-43, 46-51, 54-71, 79-89, 94-99, 102-104, 106-112, 115-164, 203-209, 212, 216-224, 234-243, 248-250, 253-270, 276, 277, 281, 282, 286-288, 299, 301, 303-309, 354, 365-368, or 375-377, wherein the AAV capsid variant comprises an amino acid sequence comprising at least one, two or three modifications, e.g., substitutions (e.g., conservative substitutions), but not more than 30, 20 or 10 modifications, e.g., substitutions (e.g., conservative substitutions), relative to the amino acid sequence of SEQ ID NO: 981.
380. The AAV particle of any one of embodiments 1-23, 26-29, 32, 35-43, 46-51, 54-71, 79-89, 94-99, 102-104, 106-112, 115-164, 203-209, 212, 216-224, 234-243, 248-250, 253-270, 276, 277, 281, 282, 286-288, 299, 301, 303-309, 354, 365-368, or 375-379, wherein the AAV capsid variant comprises an amino acid sequence comprising at least one, two or three, but not more than 30, 20 or 10 different amino acids, relative to the amino acid sequence of SEQ ID NO: 981.
381. The AAV particle of any one of embodiments 1-9, 11, 12-22, 24, 26, 28, 30, 33, 35-42, 44, 46-50, 52, 54-78, 83-88, 90-98, 100-103, 105-111, 113-129, 165-211, 213-222, 226-242, 245-249, 251-266, 271-274, 276, 278, 280, 281, 283, 285, 286, 289, 290, 300, 302-307, 331-339, 354, or 370-377, wherein the AAV capsid variant comprises the amino acid sequence of SEQ ID NO: 982, or an amino acid sequence with at least 80% (e.g., at least about 85, 90, 95, 96, 97, 98, or 99%) sequence identity thereto.
382. The AAV particle of any one of embodiments 1-9, 11, 12-22, 24, 26, 28, 30, 33, 35-42, 44, 46-50, 52, 54-78, 83-88, 90-98, 100-103, 105-111, 113-129, 165-211, 213-222, 226-242, 245-249, 251-266, 271-274, 276, 278, 280, 281, 283, 285, 286, 289, 290, 300, 302-307, 331-339, 354, 370-377, or 381, wherein the AAV capsid variant comprises an amino acid sequence comprising at least one, two or three modifications, e.g., substitutions (e.g., conservative substitutions), but not more than 30, 20 or 10 modifications, e.g., substitutions (e.g., conservative substitutions), relative to the amino acid sequence of SEQ ID NO: 982.
383. The AAV particle of any one of embodiments 1-9, 11, 12-22, 24, 26, 28, 30, 33, 35-42, 44, 46-50, 52, 54-78, 83-88, 90-98, 100-103, 105-111, 113-129, 165-211, 213-222, 226-242, 245-249, 251-266, 271-274, 276, 278, 280, 281, 283, 285, 286, 289, 290, 300, 302-307, 331-339, 354, 370-377, 381, or 382, wherein the AAV capsid variant comprises an amino acid sequence comprising at least one, two or three, but not more than 30, 20 or 10 different amino acids, relative to the amino acid sequence of SEQ ID NO: 982.
384. The AAV particle of any one of embodiments 1-383, wherein the AAV capsid variant comprises an amino acid sequence encoded by the nucleotide sequence of SEQ ID NO: 983 or 984, or a nucleotide sequence with at least 80% (e.g., at least about 85, 90, 95, 96, 97, 98, or 99%) sequence identity thereto.
385. The AAV particle of any one of the preceding embodiments 1-384, wherein the nucleotide sequence encoding the AAV capsid variant comprises the nucleotide sequence of SEQ ID NOs: 983 or 984, or a nucleotide sequence with at least 80% (e.g., at least about 85, 90, 95, 96, 97, 98, or 99%) sequence identity thereto.
386. The AAV particle of any one of the preceding embodiments 1-23, 26-29, 32, 35-43, 46-51, 54-71, 79-89, 94-99, 102-104, 106-112, 115-164, 203-209, 212, 216-224, 234-243, 248-250, 253-270, 276, 277, 281, 282, 286-288, 299, 301, 303-309, 354, 365-368, 375-380, 384, or 385, wherein the nucleotide sequence encoding the AAV capsid variant comprises the nucleotide sequence of SEQ ID NO: 983, or a nucleotide sequence with at least 80% (e.g., at least about 85, 90, 95, 96, 97, 98, or 99%) sequence identity thereto.
387. The AAV particle of any one of the preceding embodiments 1-9, 11, 12-22, 24, 26, 28, 30, 33, 35-42, 44, 46-50, 52, 54-78, 83-88, 90-98, 100-103, 105-111, 113-129, 165-211, 213-222, 226-242, 245-249, 251-266, 271-274, 276, 278, 280, 281, 283, 285, 286, 289, 290, 300, 302-307, 331-339, 354, 370-377, 381, or 382-385, wherein the nucleotide sequence encoding the AAV capsid variant comprises the nucleotide sequence of SEQ ID NO: 984, or a nucleotide sequence with at least 80% (e.g., at least about 85, 90, 95, 96, 97, 98, or 99%) sequence identity thereto.
388. The AAV particle of any one of the preceding embodiments, wherein the nucleotide sequence encoding the AAV capsid variant is codon optimized.
389. An AAV particle comprising an AAV capsid variant and a nucleic acid encoding a SOD1 targeting polynucleotide (e.g., a SOD1 targeting RNA agent, e.g., a siRNA), wherein AAV capsid variant comprising the amino acid sequence of SEQ ID NO: 981.
390. The AAV particle of embodiment 389, wherein the nucleotide sequence encoding the AAV capsid variant comprises the nucleotide sequence of SEQ ID NO: 983, or a nucleotide sequence at least 90%, 95%, or 99% identical thereto.
391. An AAV particle comprising an AAV capsid variant and a nucleic acid encoding a SOD1 targeting polynucleotide (e.g., a SOD1 targeting RNA agent, e.g., a siRNA), wherein AAV capsid variant comprising the amino acid sequence of SEQ ID NO: 982.
392. The AAV particle of embodiment 391, wherein the nucleotide sequence encoding the AAV capsid variant comprises the nucleotide sequence of SEQ ID NO: 984, or a nucleotide sequence at least 90%, 95%, or 99% identical thereto.
393. An AAV particle comprising an AAV capsid variant and a nucleic acid encoding a SOD1 targeting polynucleotide (e.g., a SOD1 targeting RNA agent, e.g., a siRNA), wherein the AAV capsid variant comprises the amino acid sequence of any one of SEQ ID NOs: 69, 70, 71, 4, 5, 6, 7, 8, or 36-59.
394. An AAV particle comprising an AAV capsid variant and a nucleic acid encoding a SOD1 targeting polynucleotide (e.g., a SOD1 targeting RNA agent, e.g., a siRNA), comprising an amino acid sequence encoded by the nucleotide sequence of any one of SEQ ID NOs: 9, 72, 73, 74, 75, 76, 77, 78, 79, 80, 81, or 22-35, or a nucleotide sequence at least 95% identical thereto.
395. The AAV particle of embodiment 393 or 394, wherein the nucleotide sequence encoding the AAV capsid variant comprises the nucleotide sequence of any one of SEQ ID NOs: 9, 72, 73, 74, 75, 76, 77, 78, 79, 80, 81, or 22-35, or a nucleotide sequence at least 95% identical thereto.
396. The AAV particle of any one of the preceding embodiments, wherein the AAV capsid variant has an increased tropism for a CNS cell or tissue, e.g., a brain cell, brain tissue, spinal cord cell, or spinal cord tissue, relative to the tropism of a reference sequence comprising the amino acid sequence of SEQ ID NO: 138.
397. The AAV particle of any one of embodiments 1-396, wherein the AAV capsid variant transduces a brain region, e.g., a putamen, caudate, entorhinal cortex, hippocampus, thalamus, substantia nigra, motor cortex, frontal cortex, temporal cortex, cerebral cortex, dentate nucleus, and/or Lateral Geniculate Nucleus (LGN), optionally wherein the level of transduction is at least 5, 10, 15, 20, 25, 30, 35, 40, 45, 50, 55, 60, or 65-fold greater as compared to a reference sequence of SEQ ID NO: 138, e.g., when measured by an assay, e.g., an immunohistochemistry assay or a qPCR assay, e.g., as described in Example 3.
398. The AAV particle of any one of embodiments 1-397, wherein the AAV capsid variant transduces a brain region, e.g., a putamen, caudate, entorhinal cortex, hippocampus, thalamus, substantia nigra, motor cortex, frontal cortex, temporal cortex, cerebral cortex, dentate nucleus, and/or Lateral Geniculate Nucleus (LGN), optionally wherein the level of transduction is at least 30, 35, 40, 45, 50, 55, 60, or 65-fold greater as compared to a reference sequence of SEQ ID NO: 138, e.g., when measured by an assay, e.g., an immunohistochemistry assay or a qPCR assay, e.g., as described in Example 3.
399. The AAV particle of any one of embodiments 1-398, wherein the AAV capsid variant is enriched at least about 3, 4, 5, 6, 7, 8, 9, or 10-fold, in the brain compared to a reference sequence of SEQ ID NO: 138, e.g., when measured by an assay as described in Example 2.
400. The AAV particle of any one of embodiments 1-399, wherein the AAV capsid variant is enriched at least about 20, 25, 30, 35, 40, 45, 50, 55, 60, 65, 70, 75, 80 or 85-fold, in the brain compared to a reference sequence of SEQ ID NO: 138, e.g., when measured by an assay as described in Example 2.
401. The AAV particle of any one of embodiments 1-400, wherein the AAV capsid variant is enriched in the brain of at least two to three species, e.g., a non-human primate and rodent (e.g., mouse), e.g., as compared to a reference sequence of SEQ ID NO: 138.
402. The AAV particle of any one of embodiments 1-401, wherein the AAV capsid variant is enriched at least about 10, 15, 20, 25, 30, 35, 40, 45, 50, 55, 60, 65, 70, 75, 80, 85, 90, 95, 100, 105, 115, 120, 125, 130, 135, 140, 145, 150, 155, 160, 165, 170, 175, 180, 190, 200, 205, or 210-fold, in the brain of at least two to three species, e.g., a non-human primate and rodent (e.g., mouse), compared to a reference sequence of SEQ ID NO: 138, e.g., when measured by an assay as described in Example 2 or 5.
403. The AAV particle of embodiment 401 or 402, wherein the at least two to three species are Macaca fascicularis, Chlorocebus sabaeus, Callithrix jacchus, and/or mouse (e.g., BALB/c mice, C57B1/6 mice, and/or CD-1 outbred mice).
404. The AAV particle of any one of embodiments 1-403, wherein the AAV capsid variant delivers an increased level of a payload to a brain region, optionally wherein the level of the payload is increased by at least 5, 10, 12, 15, 17, 18, 19, 20, 25, 30, 35, 40, 45, 50, 55, 60, 65, or 70-fold, as compared to a reference sequence of SEQ ID NO: 138, e.g., when measured by an assay, e.g., a qRT-PCR or a qPCR assay (e.g., as described in Example 3 or 7).
405. The AAV particle of any one of embodiments 1-404, wherein the AAV capsid variant delivers an increased level of viral genomes to a brain region, optionally wherein the level of viral genomes is increased by at least 5, 10, 15, 17, 18, 19, 20, 25, 30, 35, 40, 45, or 50-fold, as compared to a reference sequence of SEQ ID NO: 138, e.g., when measured by an assay, e.g., a qRT-PCR or a qPCR assay (e.g., as described in Example 3 or 7).
406. The AAV particle of any one of embodiments 1-405, wherein the AAV capsid variant is capable of transducing:
   (i) at least 20%, 25%, 30%, 40%, 50%, 55%, 60%, 65%, 70%, 75%, 80%, 85, 90%, or 95% of cells in a brain region (e.g., a putamen, caudate, entorhinal cortex, hippocampus, thalamus, substantia nigra, motor cortex, frontal cortex, temporal cortex, cerebral cortex, cerebellar cortex, cerebellum, dentate nucleus, and/or Lateral Geniculate Nucleus (LGN)), e.g., when measured by an assay as described in Example 7;
   (ii) at least 45%, 50%, 55%, 60%, 65%, 70%, 75%, 80%, 85%, 90%, 95%, 97%, or 99% of astrocytes (e.g., Sox9+astrocytes) in a brain region (e.g., a putamen, caudate, entorhinal cortex, hippocampus, thalamus, substantia nigra, motor cortex, frontal cortex, or temporal cortex), e.g., when measured by an assay as described in Example 7; and/or
   (iii) at least 25%, 30%, 35%, 40%, 45% 50%, 55%, 60%, 65%, or 70% of neurons (e.g., NeuN+neurons) in a brain region (e.g., a putamen, caudate, entorhinal cortex, hippocampus, thalamus, substantia nigra, motor cortex, frontal cortex, or temporal cortex), e.g., when measured by an assay as described in Example 7.
407. The AAV particle of any one of embodiments 404-406, wherein the brain region is a putamen, caudate, entorhinal cortex, hippocampus, thalamus, substantia nigra, motor cortex, frontal cortex, temporal cortex, cerebral cortex, dentate nucleus, and/or Lateral Geniculate Nucleus (LGN).
408. The AAV particle of any one of embodiments 1-407, wherein the AAV capsid variant is enriched at least about 5, 10, 15, 20, 25, 30, or 35-fold, in the spinal cord compared to a reference sequence of SEQ ID NO: 138, e.g., when measured by an assay as described in Example 2 or 7, optionally wherein the region of the spinal cord is a thoracic spinal cord region, cervical spinal cord region, C5 ventral horn region, lumbar spinal cord region, or L5 ventral horn region.
409. The AAV particle of any one of claims 1-408, wherein the AAV capsid variant is capable of transducing at least 70%, 75%, 80%, 85%, 90%, 91%, 92%, 93%, 94%, or 95%, 96%, or 97% of astrocytes (e.g., Sox9+astrocytes) in the spinal cord (e.g., the cervical spinal cord, the thoracic spinal cord, or the lumbar spinal cord), e.g., when measured by an assay as described in Example 7.
410. The AAV particle of any one of embodiments 1-409, wherein the AAV capsid variant shows preferential transduction in a brain region relative to the transduction in the dorsal root ganglia (DRG).
411. The AAV particle of any one of embodiments 1-410, wherein the AAV capsid variant shows preferential transduction in a brain region relative to the liver.
412. The AAV particle of any one of embodiments 1-411, wherein the AAV capsid variant shows preferential transduction in a brain region relative to the transduction in the heart.
413. The AAV particle of any one of embodiments 1-412, wherein the AAV capsid variant shows preferential transduction in a brain region relative to the transduction in the dorsal root ganglia (DRG) and the heart.
414. The AAV particle of any one of the preceding embodiments, wherein the AAV capsid variant is capable of transducing neuronal cells, e.g., NeuN+neurons or dopaminergic neurons (e.g., dopaminergic neurons in the substantia nigra), e.g., tyrosine hydroxylase (TH)+neurons.
415. The AAV particle of any one of the preceding embodiments, wherein the AAV capsid variant is capable of transducing NeuN+ cells (e.g., NeuN+neurons) and/or TH+ cells (e.g., TH+neurons, e.g., dopaminergic neurons).
416. The AAV particle of any one of the preceding embodiments, wherein the AAV capsid variant is capable of transducing non-neuronal cells, e.g., glial cells (e.g., oligodendrocytes or astrocytes).
417. The AAV particle of embodiment 416, wherein the non-neuronal cells comprise glial cells, oligodendrocytes (e.g., Olig2 positive oligodendrocytes), or astrocytes (e.g., Olig2 positive astrocytes or Sox9+astrocytes).
418. The AAV particle of any one of the preceding embodiments, wherein the AAV capsid variant is capable of transducing Olig2 positive cells, e.g., Olig2 positive astrocytes or Olig2 positive oligodendrocytes.
419. The AAV particle of any one of the preceding embodiments, wherein the AAV capsid variant is capable of transducing Sox9+ cells, e.g., Sox9+astrocytes.
420. The AAV particle of any one of the preceding embodiments, wherein the encoded SOD1 targeting polynucleotide comprises a siRNA, a shRNA, a pre-miRNA, a pri-miRNA, a miRNA, a stRNA, a lncRNA, a piRNA, an antisense oligonucleotide agent (ASO), or a snoRNA.
421. The AAV particle of embodiment 420, wherein the encoded SOD1 targeting polynucleotide comprises a siRNA comprising a sense strand and an antisense strand.
422. The AAV particle of any one of the preceding embodiments, wherein SOD1 comprises a wild type SOD1 gene, mRNA, and/or protein; a mutated SOD1 gene, mRNA, and/or protein comprising at least one mutation; or a combination thereof.
423. The AAV particle of any one of the preceding embodiments, wherein SOD1 comprises a human SOD1 gene, mRNA, and/or protein.
424. The AAV particle of any one of the preceding embodiments, wherein SOD1 comprises a primate (e.g., cynomolgus) or canine SOD1 gene, mRNA, and/or protein.
425. The AAV particle of any one of embodiments 421-424, wherein the encoded siRNA binds to a coding region of SOD1 (e.g., human SOD1, primate SOD1, or canine SOD1).
426. The AAV particle of any one of embodiments 421-425, wherein the encoded antisense strand comprises a region that is substantially complementary (e.g., at least 70%, 75%, 80%, 85%, 90%, 95%, 99% or 100% complementary) to at least part of an mRNA transcript of a SOD1 gene (e.g., a human SOD1 gene).
427. The AAV particle of any one of embodiments 421-426, wherein the encoded antisense strand comprises a region that is substantially complementary (e.g., at least 70%, 75%, 80%, 85%, 90%, 95%, 99% or 100% complementary) to at least part of a SOD1 gene (e.g., a human SOD1 gene).
428. The AAV particle of any one of embodiments 421-427, wherein the encoded antisense strand comprises a region that is substantially complementary (e.g., at least 70%, 75%, 80%, 85%, 90%, 95%, 99% or 100% complementary) to a mRNA coding region of a SOD1 gene, or a non-coding region of a SOD1 gene, e.g., a SOD1 gene comprising the nucleotide sequence of SEQ ID NO: 110 or a nucleotide sequence provided in Table 19.
429. The AAV particle of any one of embodiments 421-428, wherein the encoded antisense strand is complementary to at least 15, 16, 17, 18, 19, 20, or 21, contiguous nucleotides with 0, 1, 2, or 3 mismatches to a SOD1 sequence comprising the nucleotide sequence of SEQ ID NO: 110 or a nucleotide sequence provided in Table 19.
430. The AAV particle of any one of embodiments 421-429, wherein the encoded sense strand comprises at least 15, 16, 17, 18, 19, 20, or 21, contiguous nucleotides with 0, 1, 2, or 3 mismatches to a SOD1 sequence comprising the nucleotide sequence of SEQ ID NO: 110 or a nucleotide sequence provided in Table 19.
431. The AAV particle of any one embodiments 421-430, wherein the antisense strand comprises at least one mismatch with the target mRNA.
432. The AAV particle of any one of embodiments 426-431, wherein the region that is substantially complementary is 30 nucleotides or less (e.g., 19-24 nucleotides in length).
433. The AAV particle of any one of embodiments 421-432, wherein the encoded sense strand sequence, antisense strand sequence or both, each independently comprise 9 to 36 nucleotides in length, 15 to 30 nucleotides in length, from 15 to 25 nucleotides in length, or from 17 to 22 nucleotides in length.
434. The AAV particle of any one of embodiments 420-432, wherein the encoded siRNA comprises an antisense strand sequence comprising at least 15, 16, 17, 18, 19, 20, or 21 contiguous nucleotides differing by no more than 3, 2, 1, or 0 nucleotides from an antisense sequence in Table 10 or 14.
435. The AAV particle of any one of embodiments 420-434, wherein the encoded siRNA comprises a sense strand sequence comprising at least 15, 16, 17, 18, 19, 20, or 21 contiguous nucleotides differing by no more than 3, 2, 1, or 0 nucleotides from a sense sequence in Table 10 or 14.
436. The AAV particle of any one of embodiments 420-435, wherein the encoded siRNA comprises:
   (i) a sense strand sequence comprising at least 15, 16, 17, 18, 19, 20, or 21 contiguous nucleotides differing by no more than 3, 2, 1, or 0 nucleotides from a sense sequence in Table 10 or 14; and
   (ii) an antisense strand sequence comprising at least 15, 16, 17, 18, 19, 20, or 21 contiguous nucleotides differing by no more than 3, 2, 1, or 0 nucleotides from an antisense sequence in Table 10 or 14;
wherein the sense strand sequence and the antisense strand sequence comprise a region of complementarity of at least 15 nucleotides.
437. The AAV particle of any one of embodiments 420-433, wherein the encoded siRNA comprises:
   (i) a sense strand sequence from Table 10 or 14, or a nucleotide at least 80%, 81%, 82%, 83%, 84%, 85%, 86%, 87%, 88%, 89%, 90%, 91%, 92%, 93%, 94%, 95%, 96%, 97%, 98% or 99%, identical to a sense strand sequence in Table 10; and/or
   (ii) an antisense strand sequence from Table 10 or 14, or a nucleotide at least 80%, 81%, 82%, 83%, 84%, 85%, 86%, 87%, 88%, 89%, 90%, 91%, 92%, 93%, 94%, 95%, 96%, 97%, 98% or 99%, identical to an antisense strand sequence in Table 10 or 14;
wherein the sense strand sequence and the antisense strand sequence comprise a region of complementarity of at least 15 nucleotides.
438. The AAV particle of any one of embodiments 420-437, wherein the encoded siRNA comprises an antisense strand sequence comprising at least 15, 16, 17, 18, 19, 20, or 21 contiguous nucleotides differing by no more than 3, 2, 1, or 0 nucleotides from SEQ ID NO: 2507, 2365, or 2363.
439. The AAV particle of any one of embodiments 420-438, wherein the encoded siRNA comprises a sense strand sequence comprising at least 15, 16, 17, 18, 19, 20, or 21 contiguous nucleotides differing by no more than 3, 2, 1, or 0 nucleotides from any one of SEQ ID NOs: 2525, 2376, or 2381.
440. The AAV particle of any one of embodiments 420-439, wherein the encoded siRNA comprises:
   (i) a sense strand sequence comprising at least 15, 16, 17, 18, 19, 20, or 21 contiguous nucleotides differing by no more than 3, 2, 1, or 0 nucleotides from SEQ ID NO: 2525, 2376, or 2381; and
   (ii) an antisense strand sequence comprising at least 15, 16, 17, 18, 19, 20, or 21 contiguous nucleotides differing by no more than 3, 2, 1, or 0 nucleotides from SEQ ID NO: 2507, 2365, or 2363; wherein the sense strand sequence and the antisense strand sequence comprise a region of complementarity of at least 15 nucleotides.
441. The AAV particle of any one of embodiments 420-440, wherein the encoded sense strand sequence comprises at least 18, 19, 20, or 21 contiguous nucleotides differing by no more than 3, 2, 1 or 0 nucleotides from SEQ ID NO: 2525 or 2381.
442. The AAV particle of any one of embodiments 420-441, wherein the encoded antisense strand sequence comprises at least 18, 19, 20, or 21 contiguous nucleotides differing by no more than 3, 2, 1 or 0 nucleotides from SEQ ID NO: 2507 or 2363.
443. The AAV particle of any one of embodiments 420-442, wherein:
   (i) the encoded sense strand sequence comprises SEQ ID NO: 2525, or at least 18, 19, 20, or 21 contiguous nucleotides differing by no more than 3, 2, 1 or 0 nucleotides from SEQ ID NO: 2525, and the encoded antisense strand sequence comprises at least 18, 19, 20, or 21 contiguous nucleotides differing by no more than 3, 2, 1 or 0 nucleotides SEQ ID NO: 2507; or
   (iv) the encoded sense strand sequence comprises at least 18, 19, 20, or 21 contiguous nucleotides differing by no more than 3, 2, 1 or 0 nucleotides SEQ ID NO: 2381, and the encoded antisense strand sequence comprises at least 18, 19, 20, or 21 contiguous nucleotides differing by no more than 3, 2, 1 or 0 nucleotides SEQ ID NO: 2363.
444. The AAV particle of any one of embodiments 420-443, wherein the encoded siRNA comprises:
   (i) a sense strand sequence comprising SEQ ID NO: 2525, or a nucleotide differing by no more than 3, 1, or 0 nucleotides from SEQ ID NO: 2525, and an antisense strand sequence comprising SEQ ID NO: 2507, or a nucleotide differing by no more than 3 (e.g., by no more than 0, 1 or 2) nucleotides from SEQ ID NO: 2507;
   (ii) a sense strand sequence comprising SEQ ID NO: 2376, or a nucleotide differing by no more than 3, 2, 1, or 0 nucleotides from SEQ ID NO: 2376, and an antisense strand sequence comprising SEQ ID NO: 2365, or a nucleotide differing by no more than 3 (e.g., by no more than 0, 1 or 2) nucleotides from SEQ ID NO: 2365; and/or
   (iii) a sense strand sequence comprising SEQ ID NO: 2381, or a nucleotide differing by no more than 3, 2, 1, or 0 nucleotides from SEQ ID NO: 2381, and an antisense strand sequence comprising SEQ ID NO: 2363, or a nucleotide differing by no more than 3 (e.g., by no more than 0, 1 or 2) nucleotides from SEQ ID NO: 2363.
445. The AAV particle of any one of embodiments 420-444, wherein the encoded siRNA comprises:
   (i) a sense strand sequence comprising SEQ ID NO: 2525, and an antisense strand sequence comprising SEQ ID NO: 2507;
   (ii) a sense strand sequence comprising SEQ ID NO: 2376, and an antisense strand sequence comprising SEQ ID NO: 2365; and/or
   (iii) a sense strand sequence comprising SEQ ID NO: 2381, and an antisense strand sequence comprising SEQ ID NO: 2363.
446. The AAV particle of any one of embodiments 420-445, wherein the encoded siRNA comprises a sense strand sequence comprising SEQ ID NO: 2525, and an antisense strand sequence comprising SEQ ID NO: 2507.
447. The AAV particle of any one of embodiments 421-446, wherein:
   (i) the nucleotide sequence encoding the sense strand sequence comprises SEQ ID NO: 66; and
   (ii) the nucleotide sequence encoding the sense strand sequence comprises SEQ ID NO: 1351.
448. An AAV particle comprising:
   (i) an AAV capsid variant comprising the amino acid sequence of SEQ ID NO: 69; and
   (ii) a nucleic acid encoding a polynucleotide targeting human SOD1, wherein the encoded polynucleotide comprises a siRNA comprising a sense strand sequence and an antisense strand sequence, wherein the encoded sense strand sequence comprises SEQ ID NO: 2525, and the encoded antisense strand sequence comprises SEQ ID NO: 2507.
449. An AAV particle comprising:
   (i) an AAV capsid variant comprising the amino acid sequence of SEQ ID NO: 36; and
   (ii) a nucleic acid encoding a polynucleotide targeting human SOD1, wherein the encoded polynucleotide comprises a siRNA comprising a sense strand sequence and an antisense strand sequence, wherein the encoded sense strand sequence comprises SEQ ID NO: 2525, and the encoded antisense strand sequence comprises SEQ ID NO: 2507.
450. An AAV particle comprising:
   (i) an AAV capsid variant comprising the amino acid sequence of SEQ ID NO: 982; and
   (ii) a nucleic acid encoding a polynucleotide targeting human SOD1, wherein the encoded polynucleotide comprises a siRNA comprising a sense strand sequence and an antisense strand sequence, wherein the encoded sense strand sequence comprises SEQ ID NO: 2525, and the encoded antisense strand sequence comprises SEQ ID NO: 2507.
451. The AAV particle of any one of embodiments 421-450, wherein the sense strand sequence and the antisense strand sequence comprise a region of complementarity, of at least 17-20 nucleotides in length.
452. The AAV particle of any one of embodiments 421-451, wherein at least one of the sense strand sequence and the antisense strand sequence comprise a 3′ overhang of at least 1, or 2 nucleotides.
453. The AAV particle of any one of embodiments 421-452, wherein sense strand and/or antisense strand are at least 20, 21, or 22 nucleotides in length.
454. The AAV particle of any one of embodiments 420-453, wherein the encoded SOD1 targeting polynucleotide further comprises modulatory polynucleotide comprising a siRNA.
455. The AAV particle of embodiment 454, wherein the encoded modulatory polynucleotide comprises:
   (i) a 5′ flanking region;
   (ii) a loop region; and/or
   (iii) a 3′ flanking region.
456. The AAV particle of embodiment 455, wherein:
   (i) the encoded 5′ flanking region comprises the nucleotide sequence of any one of SEQ ID NOs: 5000-5003; a nucleotide sequence at least 70%, 75%, 80%, 85%, 90%, 92%, 95%, 97%, 98%, or 99% identical to the nucleotide sequence of any one of SEQ ID NOs: 5000-5003; or a nucleotide sequence having at least one, two, three, four, five, six, or seven modifications, but no more than ten modifications relative to the nucleotide sequence of any one of SEQ ID NO: 5000-5003;
   (ii) the encoded loop region comprises the nucleotide sequence of any one of SEQ ID NOS: 5004-5007; a nucleotide sequence at least 70%, 75%, 80%, 85%, 90%, 92%, 95%, 97%, 98%, or 99% identical to the nucleotide sequence of any one of SEQ ID NOs: 5004-5007; or a nucleotide sequence having at least one, two, three, or four, modifications, but no more than six modifications relative to the nucleotide sequence of any one of SEQ ID NOs: 5004-5007; and/or
   (iii) the encoded 3′ flanking region comprises the nucleotide sequence any one of SEQ ID NOs: 5008-5012; a nucleotide sequence at least 70%, 75%, 80%, 85%, 90%, 92%, 95%, 97%, 98%, or 99% identical to the nucleotide sequence of any one of SEQ ID NOs: 5008-5012; or a nucleotide sequence having at least one, two, three, four, five, six, or seven modifications, but no more than ten modifications relative to the nucleotide sequence of any one of SEQ ID NOs: 5008-5012.
457. The AAV particle of embodiment 455 or 456, wherein:
   (i) the nucleotide sequence encoding the 5′ flanking region comprises the nucleotide sequence of any one of SEQ ID NOs: 2547-2549 or 5014; a nucleotide sequence at least 70%, 75%, 80%, 85%, 90%, 92%, 95%, 97%, 98%, or 99% identical to the nucleotide sequence of any one of SEQ ID NOs: 2547-2549 or 5014; or a nucleotide sequence having at least one, two, three, four, five, six, or seven modifications, but no more than ten modifications relative to the nucleotide sequence of any one of SEQ ID NO: 2547, 2548, 2549, or 5014;
   (ii) the nucleotide sequence encoding the loop region comprises the nucleotide sequence of any one of SEQ ID NOs: 2550-2553; a nucleotide sequence at least 70%, 75%, 80%, 85%, 90%, 92%, 95%, 97%, 98%, or 99% identical the nucleotide sequence of any one of SEQ ID NOs: 2550-2553; or a nucleotide sequence having at least one, two, three, or four, modifications, but no more than six modifications relative to the nucleotide sequence of any one of SEQ ID NOs: 2550-2553; and/or
   (iii) the nucleotide sequence encoding the 3′ flanking region comprises the nucleotide sequence of any one of SEQ ID NOs: 2554-2558; a nucleotide sequence at least 70%, 75%, 80%, 85%, 90%, 92%, 95%, 97%, 98%, or 99% identical to the nucleotide sequence of any one of SEQ ID NOs: 2554-2558; or a nucleotide sequence having at least one, two, three, four, five, six, or seven modifications, but no more than ten modifications relative to the nucleotide sequence of any one of SEQ ID NOs: 2554-2558.
458. The AAV particle of any one of embodiments 455-457, wherein:
   (i) the encoded 5′ flanking region comprises a nucleotide sequence of SEQ ID NO: 5000, 5001, or 5002; or a nucleotide sequence having at least one, two, three, four, five, six, or seven modifications, but no more than ten modifications relative to the nucleotide sequence of SEQ ID NO: 5000, 5001, or 5002;
   (ii) the encoded loop region comprises a nucleotide sequence of any one of SEQ ID NOs: 5004, 5005, or 5006, or a nucleotide sequence having at least one, two, three, or four, modifications, but no more than six modifications relative to the nucleotide sequence of any one of SEQ ID NOs: 5004, 5005, or 5006; and/or
   (iii) 3′ flanking region comprises a nucleotide sequence of any one of SEQ ID NOs: 5008, 5009, or 5012, or a nucleotide sequence having at least one, two, three, four, five, six, or seven modifications, but no more than ten modifications relative to the nucleotide sequence of any one of SEQ ID NO: 5008, 5009, or 5012.
459. The AAV particle of any one of embodiments 455-458, wherein:
   (i) the nucleotide sequence encoding the 5′ flanking region comprises the nucleotide sequence of SEQ ID NO: 2547, 2548, or 2549; a nucleotide sequence having at least one, two, three, four, five, six, or seven modifications, but no more than ten modifications relative to the nucleotide sequence of SEQ ID NO: 2547, 2548, or 2549;
   (ii) the nucleotide sequence encoding the loop region comprises the nucleotide sequence of any one of SEQ ID NOs: 2550, 2551, or 2552; or a nucleotide sequence having at least one, two, three, or four, modifications, but no more than six modifications relative to the nucleotide sequence of any one of SEQ ID NOs: 2550, 2551, or 2552; and/or
   (iii) the nucleotide sequence encoding the 3′ flanking region comprises the nucleotide sequence of any one of SEQ ID NO: 2554, 2555, or 2558; or a nucleotide sequence having at least one, two, three, four, five, six, or seven modifications, but no more than ten modifications relative to the nucleotide sequence of any one of SEQ ID NOs: 2554, 2555, or 2558.
460. The AAV particle of any one of embodiments 455-459, wherein:
   (i) the encoded 5′ flanking region comprises the nucleotide sequence of SEQ ID NO: 5000, or a nucleotide sequence at least 80%, 85%, 90%, 95%, 97%, 98%, or 99% identical thereto;
   (ii) the encoded loop region comprises the nucleotide sequence of SEQ ID NO: 5004, or a nucleotide sequence at least 80%, 85%, 90%, 95%, 97%, 98%, or 99% identical thereto; and/or
   (iii) the encoded 3′ flanking region comprises the nucleotide sequence of SEQ ID NO: 5008, or a nucleotide sequence at least 80%, 85%, 90%, 95%, 97%, 98%, or 99% identical thereto.
461. The AAV particle of any one of embodiments 455-459, wherein:
   (i) the nucleotide sequence encoding the 5′ flanking region comprises SEQ ID NO: 2547, or a nucleotide sequence at least 80%, 85%, 90%, 95%, 97%, 98%, or 99% identical thereto;
   (ii) the nucleotide sequence encoding the loop region comprises SEQ ID NO: 2550, or a nucleotide sequence at least 80%, 85%, 90%, 95%, 97%, 98%, or 99% identical thereto; and/or
   (iii) the nucleotide sequence encoding the 3′ flanking region comprises SEQ ID NO: 2554, or a nucleotide sequence at least 80%, 85%, 90%, 95%, 97%, 98%, or 99% identical thereto.
462. The AAV particle of any one of embodiments 454-461, wherein the encoded modulatory polynucleotide comprises the nucleotide sequence of SEQ ID NO: 5013, or a nucleotide sequence at least 70%, 75%, 80%, 85%, 90%, 92%, 95%, 97%, 98%, or 99% identical thereto.
463. The AAV particle of any one of embodiments 454-462, wherein:
   (i) the encoded modulatory polynucleotide comprises the nucleotide sequence of SEQ ID NO: 4821, or a nucleotide sequence at least 70%, 75%, 80%, 85%, 90%, 92%, 95%, 97%, 98%, or 99% identical thereto; or
   (ii) the nucleotide sequence encoding the modulatory polynucleotide comprises any one of SEQ ID NOs: 2562, 2579, 5022, 2559, 2560, 2561, 2563, 2564, 2565, 2566, 2567, 2568, 2569, 2570, 2571, 2572, 2573, 2574, 2575, 2576, 2577, 2578 or a nucleotide sequence at least 70%, 75%, 80%, 85%, 90%, 92%, 95%, 97%, 98%, or 99% identical thereto.
464. The AAV particle of any one of embodiments 454-463, wherein the nucleotide sequence encoding the modulatory polynucleotide comprises any one of SEQ ID NOs: 2562, 2579, or 5022, or a nucleotide sequence at least 70%, 75%, 80%, 85%, 90%, 92%, 95%, 97%, 98%, or 99% identical thereto.
465. The AAV particle of any one of embodiments 454-464, wherein the nucleotide sequence encoding the modulatory polynucleotide comprises the nucleotide sequence of SEQ ID NO: 2562 or a nucleotide sequence at least 70%, 75%, 80%, 85%, 90%, 92%, 95%, 97%, 98%, or 99% identical thereto.
466. An AAV particle comprising:
   (i) an AAV capsid variant comprising the amino acid sequence of SEQ ID NO: 69; and
   (ii) a nucleic acid encoding a polynucleotide targeting human SOD1, wherein the encoded polynucleotide comprises the nucleotide sequence of SEQ ID NO: 5013.
467. An AAV particle comprising:
   (i) an AAV capsid variant comprising the amino acid sequence of SEQ ID NO: 36; and
   (ii) a nucleic acid encoding a polynucleotide targeting human SOD1, wherein the encoded polynucleotide comprises the nucleotide sequence of SEQ ID NO: 5013.
468. An AAV particle comprising:
   (i) an AAV capsid variant comprising the amino acid sequence of SEQ ID NO: 982; and
   (ii) a nucleic acid encoding a polynucleotide targeting human SOD1, wherein the encoded polynucleotide comprises the nucleotide sequence of SEQ ID NO: 5013.
469. The AAV particle of any one of the preceding embodiments, which comprises a viral genome comprising a promoter operably linked to the nucleic acid sequence encoding the SOD1 targeting polynucleotide (e.g., a SOD1 targeting RNA agent, e.g., a siRNA).
470. The AAV particle of claim 469, wherein the promoter is a ubiquitous promoter, 471. The AAV particle of claim 469, wherein the promoter is a tissue specific promoter.
472. The AAV particle of any one of embodiments 469-471, wherein the promoter is chosen from an H1 promoter, human elongation factor 1α-subunit (EF1α), cytomegalovirus (CMV) immediate-early enhancer and/or promoter, chicken β-actin (CBA) and its derivative CAG, β glucuronidase (GUSB), or ubiquitin C (UBC), neuron-specific enolase (NSE), platelet-derived growth factor (PDGF), platelet-derived growth factor B-chain (PDGF-β), intercellular adhesion molecule 2 (ICAM-2), synapsin (Syn), methyl-CpG binding protein 2 (MeCP2), Ca2+/calmodulin-dependent protein kinase II (CaMKII), metabotropic glutamate receptor 2 (mGluR2), neurofilament light (NFL) or heavy (NFH), β-globin minigene nβ2, preproenkephalin (PPE), enkephalin (Enk) and excitatory amino acid transporter 2 (EAAT2), glial fibrillary acidic protein (GFAP), myelin basic protein (MBP), phosphoglycerate kinase (PGK), insulin, a cardiovascular promoter (e.g., αMHC, cTnT, and CMV-MLC2k), a liver promoter (e.g., hAAT, TBG), a skeletal muscle promoter (e.g., desmin, MCK, C512) or a fragment, e.g., a truncation, or a functional variant thereof.
473. The AAV particle of any one of embodiments 469-472, wherein the promoter is an H1 promoter, EF-1a promoter variant, e.g., a truncated EF-1a promoter, a CBA promoter variant, e.g., a truncated CBA promoter, an insulin promoter variant, e.g., a truncated insulin promoter, or a SYN promoter variant, e.g., a truncated SYN promoter.
474. The AAV particle of any one of embodiments 469, 470, 472, or 473, wherein the promoter is an H1 promoter.
475. The AAV particle of any one of embodiments 469, 470, 472, or 474, wherein the promoter comprises the nucleotide sequence of any one of SEQ ID NOs: 4000-4026, a nucleotide sequence comprising at least one, two, or three but no more than four modifications, e.g., substitutions (e.g., conservative substitutions), insertions, or deletions, relative to the nucleotide sequence of SEQ ID NOs: 4000-4026, or a nucleotide sequence with at least 80% (e.g., 85%, 90%, 95%, 96%, 97%, 98%, or 99%) sequence identity to any one of SEQ ID NOs: 4000-4026.
476. The AAV particle of any one of embodiments 469, 470, 472, or 474, wherein the promoter is a H1 promoter comprising SEQ ID NO: 128 or SEQ ID NO: 83, or a nucleotide sequence with at least 80% (e.g., 85%, 90%, 95%, 96%, 97%, 98%, or 99%) sequence identity to SEQ ID NO: 128 or SEQ ID NO: 83.
477. The AAV particle of any one of embodiments 469-476, wherein the viral genome further comprises a first exon, a second exon, or both a first exon and second exon.
478. The AAV particle of any one of embodiments 477, wherein the first or second exon comprises the nucleotide sequence of any one of SEQ ID NO: 4045-4048, a nucleotide sequence comprising at least one, two, or three but no more than four modifications, e.g., substitutions, insertions, or deletions, relative to the nucleotide sequence of any one of SEQ ID NO: 4045-4048, or a nucleotide sequence with at least 80% (e.g., 85%, 90%, 95%, 96%, 97%, 98%, or 99%) sequence identity to any one of SEQ ID NO: 4045-4048.
479. The AAV particle of any one of embodiments 477 or 478, wherein the viral genome comprises both a first exon and a second exon, wherein:
   (a) the first exon comprises the nucleotide sequence of SEQ ID NO: 4045 or 4046, a nucleotide sequence comprising at least one, two, or three but no more than four modifications, e.g., substitutions, insertions, or deletions, relative to the nucleotide sequence of SEQ ID NO: 4045 or 4046, or a nucleotide sequence with at least 80% (e.g., 85%, 90%, 95%, 96%, 97%, 98%, or 99%) sequence identity to SEQ ID NO: 4045 or 4046; and
   (b) the second exon comprises the nucleotide sequence of SEQ ID NO: 4047 or 4048, a nucleotide sequence comprising at least one, two, or three but no more than four modifications, e.g., substitutions, insertions, or deletions, relative to the nucleotide sequence of SEQ ID NO: 4047 or 4048, or a nucleotide sequence with at least 80% (e.g., 85%, 90%, 95%, 96%, 97%, 98%, or 99%) sequence identity to SEQ ID NO: 4047 or 4048.
480. The AAV particle of any one of embodiments 477-479, wherein the first exon, second exon, or both are codon optimized.
481. The AAV particle of any one of embodiments 477-480, wherein one or more or all of the CpG sequences in the first exon, second exon, or both are depleted.
482. The AAV particle of any one of embodiments 477-481, wherein the viral genome further comprises an intron.
483. The AAV particle of embodiment 482, wherein the intron is located 3′ relative to (a) a 5′ ITR, (b) the promoter, and/or (c) the first exon.
484. The AAV particle of embodiment 482 or 483, wherein the intron is located 5′ relative to (a) the second exon, (b) the modulatory polynucleotide, (c) a poly A signal sequence, and/or (d) a 3′ITR.
485. The AAV particle of any one of embodiments 469-484, wherein the viral genome comprises a first exon, an intron, and a second exon, wherein the intron is located 3′ relative to the first exon and 5′ relative to the second exon.
486. The AAV particle of any one of embodiments 482-485, wherein the intron comprises the nucleotide sequence of SEQ ID NO: 4044, a nucleotide sequence comprising at least one, two, or three but no more than four modifications, e.g., substitutions, insertions, or deletions, relative to the nucleotide sequence of SEQ ID NO: 4044, or a nucleotide sequence with at least 80% (e.g., 85%, 90%, 95%, 96%, 97%, 98%, or 99%) sequence identity to SEQ ID NO: 4044.
487. The AAV particle of any one of embodiments 469-486, wherein the viral genome comprises a first exon, an intron, and second exon (“exon-intron-exon cassette”).
488. The AAV particle of embodiment 487, wherein the exon-intron-exon cassette comprises the nucleotide sequence of SEQ ID NO: 4042 or 4043, a nucleotide sequence comprising at least one, two, or three but no more than four modifications, e.g., substitutions, insertions, or deletions, relative to the nucleotide sequence of SEQ ID NO: 4042 or 4043, or a nucleotide sequence with at least 80%, 85%, 90%, 95%, 96%, 97%, 98%, or 99% sequence identity to SEQ ID NO: 4042 or 4043.
489. The AAV particle of embodiment 487 or 488, wherein the exon-intron-exon cassette is positioned 3′ relative to the promoter, and 5′ relative to the nucleotide sequence encoding the modulatory polynucleotide (e.g., a SOD1 targeting RNA agent, e.g., a siRNA).
490. The AAV particle of any one of embodiments 487-489, wherein the nucleotide sequence encoding the modulatory polynucleotide (e.g., a SOD1 targeting RNA agent, e.g., a siRNA) is present in (e.g., inserted into) the intron of the exon-intron-exon cassette.
491. The AAV particle of any one of embodiments 487-490, wherein the nucleotide sequence encoding the modulatory polynucleotide (e.g., a SOD1 targeting RNA agent, e.g., a siRNA) replaces a portion or all of the intron in the exon-intron-exon cassette.
492. The AAV particle of any one of embodiments 469-491, wherein the viral genome further comprises a polyA signal sequence.
493. The AAV particle of embodiment 492, wherein the polyA signal sequence comprises SEQ ID NO: 129 or 4027, or a nucleotide sequence with at least 80%, 85%, 90%, 95%, 96%, 97%, 98%, or 99% sequence identity to SEQ ID NO: 129 or 4027.
494. The AAV particle of any one of embodiments 469-493, wherein the viral genome further comprises an inverted terminal repeat (ITR) sequence.
495. The AAV particle of any one of embodiments 469-494, wherein the viral genome comprises an ITR sequence positioned 5′ relative to the encoded SOD1 targeting polynucleotide.
496. The AAV particle of any one of embodiments 469-495, wherein the viral genome comprises an ITR sequence positioned 3′ relative to the encoded SOD1 targeting polynucleotide.
497. The AAV particle of any one of embodiments 469-496, wherein the viral genome comprises an ITR sequence positioned 5′ relative to the encoded SOD1 targeting polynucleotide and an ITR sequence positioned 3′ relative to the encoded SOD1 targeting polynucleotide.
498. The AAV particle of embodiment 497, wherein the ITR sequence positioned 5′ relative to the encoded SOD1 targeting polynucleotide and an ITR sequence positioned 3′ relative to the SOD1 targeting polynucleotide, comprise SEQ ID NOs: 126 or 130, or a nucleotide sequence with at least 80%, 85%, 90%, 95%, 96%, 97%, 98%, or 99% sequence identity thereto.
499. The AAV particle of embodiment 497 or 498, wherein:
   (i) the ITR sequence positioned 5′ relative to the encoded SOD1 targeting polynucleotide comprises the nucleotide sequence of SEQ ID NO: 126, or a nucleotide sequence at least 80%, 85%, 90%, 95%, 96%, 97%, 98%, or 99% identical thereto; and/or
   (ii) the ITR sequence positioned 3′ relative to the encoded SOD1 targeting polynucleotide comprises the nucleotide sequence of SEQ ID NO: 130, or a nucleotide sequence at least 80%, 85%, 90%, 95%, 96%, 97%, 98%, or 99% identical thereto.
500. The AAV particle of any one of embodiments 469-499, wherein the viral genome further comprises a filler sequence.
501. The AAV particle of embodiment 500, wherein the filler sequence comprises the nucleotide sequence of SEQ ID NO: 82, or a nucleotide sequence at least 70%, 75%, 80%, 85%, 90%, 95%, 96%, 97%, 98%, or 99% identical thereto.
502. The AAV particle of any one of embodiments 469-501, wherein the viral genome further comprises an enhancer, a Kozak sequence, an intron region, and/or an exon region.
503. The AAV particle of any one of embodiments 469-502, wherein the viral genome comprises, from 5′ to 3′:
   (a) a 5′ ITR, e.g., a 5′ITR comprising the nucleotide sequence of SEQ ID NO: 126 or 130 or variant thereof;
   (b) a promoter, e.g., a promoter comprising the nucleotide sequence of any one of SEQ ID NOs: 4000-4026 or variant thereof;
   (c) an exon, e.g., an exon comprising the nucleotide sequence of any one of SEQ ID NOs: 4045-4048 or variant thereof;
   (d) a nucleotide sequence encoding a modulatory polynucleotide, e.g., a nucleotide sequence encoding the modulatory polynucleotide of any one of SEQ ID NOs: 2559-2579 and 5022 or variant thereof;
   (e) a poly A signal sequence, e.g., a poly A signal sequence comprising the nucleotide sequence of SEQ ID NO: 129 or 4027 or variant thereof, and
   (f) a 3′ITR, e.g., a 3′ITR comprising the nucleotide sequence of SEQ ID NO: 126 or 130 or variant thereof;
   wherein the variant of any of (a)-(f) comprises a sequence differing by no more than 15, 14, 13, 12, 11, 10, 9, 8, 7, 6, 5, 4, 3, 2 or 1 nucleotides, e.g., substitutions, insertions, or deletions, relative to the reference sequence, or a sequence with at least 70%, 75%, 80%, 85%, 90%, 95%, 99% or 100% identity, relative to the reference sequence, e.g., wherein the reference sequence is a wild-type sequence.
504. The AAV particle of any one of embodiments 469-503, wherein the viral genome comprises, from 5′ to 3′:
   (a) a 5′ ITR, e.g., a 5′ITR comprising the nucleotide sequence of SEQ ID NO: 126 or 130 or variant thereof,
   (b) a promoter, e.g., a promoter comprising the nucleotide sequence of any one of SEQ ID NOs: 4000-4026 or variant thereof,
   (c) a first exon, e.g., a first exon comprising the nucleotide sequence of any one of SEQ ID NOS: 4045-4048 or variant thereof,
   (d) an intron, e.g., an intron comprising the nucleotide sequence of SEQ ID NO: 4044 or variant thereof,
   (e) a nucleotide sequence encoding a modulatory polynucleotide, e.g., a nucleotide sequence encoding the modulatory polynucleotide of any one of SEQ ID NOs: 2559-2579 and 5022 or variant thereof,
   (f) a poly A signal sequence, e.g., a poly A signal sequence comprising the nucleotide sequence of SEQ ID NO: 129 or 4027 or variant thereof, and
   (g) a 3′ITR, e.g., a 3′ITR comprising the nucleotide sequence of SEQ ID NO: 126 or 130 or variant thereof,
   wherein the variant comprises a sequence differing by no more than 15, 14, 13, 12, 11, 10, 9, 8, 7, 6, 5, 4, 3, 2 or 1 nucleotides, e.g., substitutions, insertions, or deletions, relative to the reference sequence, or a sequence with at least 70%, 75%, 80%, 85%, 90%, 95%, 99% or 100% identity relative to the reference sequence, e.g., wherein the reference sequence is a wild-type sequence.
505. The AAV particle of any one of embodiments 469-504, wherein the viral genome comprises, from 5′ to 3′:
   (a) a 5′ ITR, e.g., a 5′ITR comprising the nucleotide sequence of SEQ ID NO: 126 or 130 or variant thereof,
   (b) a promoter, e.g., a promoter comprising the nucleotide sequence of any one of SEQ ID NOs: 4000-4026 or variant thereof,
   (c) a first exon, e.g., a first exon comprising the nucleotide sequence of any one of SEQ ID NOs: 4045-4048 or variant thereof,
   (d) an intron, e.g., an intron comprising the nucleotide sequence of SEQ ID NO: 4044 or variant thereof,
   (e) a second exon, e.g., a second exon comprising the nucleotide sequence of any one of SEQ ID NOs: 4045-4048 or variant thereof,
   (f) a nucleotide sequence encoding a modulatory polynucleotide, e.g., a nucleotide sequence encoding the modulatory polynucleotide of any one of SEQ ID NOs: 2559-2579 and 5022 or variant thereof,
   (g) a poly A signal sequence, e.g., a poly A signal sequence comprising the nucleotide sequence of SEQ ID NO: 129 or 4027 or variant thereof, and
   (h) a 3′ITR, e.g., a 3′ITR comprising the nucleotide sequence of SEQ ID NO: 126 or 130 or variant thereof,
   wherein the variant comprises a sequence differing by no more than 15, 14, 13, 12, 11, 10, 9, 8, 7, 6, 5, 4, 3, 2 or 1 nucleotides, e.g., substitutions, insertions, or deletions, relative to the reference sequence, or a sequence with at least 70%, 75%, 80%, 85%, 90%, 95%, 99% or 100% identity relative to the reference sequence, e.g., wherein the reference sequence is a wild-type sequence.
506. The AAV particle of any one of embodiments 469-505, wherein the viral genome further comprises a nucleotide sequence encoding a miR binding site, e.g., a miR binding site that modulates, e.g., reduces, expression of the SOD1 targeting polynucleotide encoded by the viral genome in a cell or tissue where the corresponding miRNA is expressed.
507. The AAV particle of embodiment 506, wherein the encoded miRNA binding site is complementary, e.g., fully complementary or partially complementary, to a miRNA expressed in a cell or tissue of the DRG, liver, heart, hematopoietic, or a combination thereof.
508. The AAV particle of embodiment 506 507, wherein the encoded miR binding site modulates, e.g., reduces, expression of the encoded SOD1 targeting polynucleotide in a cell or tissue of the DRG, liver, heart, hematopoietic lineage, or a combination thereof.
509. The AAV particle of any one of embodiments 506-508, wherein the viral genome comprises at least 1-5 copies of the encoded miR binding site, e.g., at least 1, 2, 3, 4, or 5 copies.
510. The AAV particle of any one of embodiments 506-509, wherein the viral genome comprises at least 3 copies of an encoded miR binding sites, optionally wherein all three copies comprise the same miR binding site, or at least one, two, three, or all of the copies comprise a different miR binding site.
511. The AAV particle of embodiment 510, wherein the at least 3 copies of the encoded miR binding sites are continuous (e.g., not separated by a spacer), or are separated by a spacer, optionally wherein the spacer comprises the nucleotide sequence GATAGTTA, or a nucleotide sequence having at least one, two, or three modifications, e.g., substitutions, but no more than four modifications, e.g., substitutions, relative to GATAGTTA.
512. The AAV particle of any one of embodiments 407-415, wherein the viral genome comprises at least 4 copies of an encoded miR binding site, optionally wherein all four copies comprise the same miR binding site, or at least one, two, three, or all of the copies comprise a different miR binding site.
513. The AAV particle of embodiment 512, wherein the at least 4 copies of the encoded miR binding sites are continuous (e.g., not separated by a spacer), or are separated by a spacer, optionally wherein the spacer comprises the nucleotide sequence GATAGTTA, or a nucleotide sequence having at least one, two, or three modifications, e.g., substitutions, but no more than four modifications, e.g., substitutions, relative to GATAGTTA.
514. The AAV particle of any one of embodiments 506-513, wherein the encoded miR binding site comprises a miR 122 binding site, a miR183 binding site, a miR-1 binding site, a miR-142-3p, or a combination thereof, optionally wherein:
   (i) the encoded miR122 binding site comprises the nucleotide sequence of SEQ ID NO: 61, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions, but no more than ten modifications, e.g., substitutions, relative to SEQ ID NO: 61;
   (ii) the encoded miR183 binding site comprises the nucleotide sequence of SEQ ID NO: 60, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions, but no more than ten modifications, e.g., substitutions, relative to SEQ ID NO: 60;
   (iii) the encoded miR-1 binding site comprises the nucleotide sequence of SEQ ID NO: 4679, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions, but no more than ten modifications, e.g., substitutions, relative to SEQ ID NO: 4679; and/or
   (iv) the encoded miR-142-3p binding site comprises the nucleotide sequence of SEQ ID NO: 65, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions, but no more than ten modifications, e.g., substitutions, relative to SEQ ID NO: 65.
515. The AAV particle of any one of embodiments 469-514, wherein the viral genome comprises an encoded miR 122 binding site.
516. The AAV particle of any one of embodiments 469-515, wherein the viral genome comprises at least 1-5 copies, e.g., 1, 2, or 3 copies of a miR122 binding site, optionally wherein each copy is continuous (e.g., not separated by a spacer), or each copy is separated by a spacer, optionally wherein the spacer comprises the nucleotide sequence GATAGTTA, or a nucleotide sequence having at least one, two, or three modifications, e.g., substitutions, but no more than four modifications, e.g., substitutions, relative to GATAGTTA.
517. The AAV particle of any one of embodiments 514-516, wherein the encoded miR122 binding site comprises the nucleotide sequence of SEQ ID NO: 61, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions, but no more than ten modifications, e.g., substitutions, relative to SEQ ID NO: 61.
518. The AAV particle of any one of embodiments 469-517, wherein the viral genome comprises: (A) (i) a first encoded miR122 binding site comprising the nucleotide sequence of SEQ ID NO: 61, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions, but no more than ten modifications, e.g., substitutions, relative to SEQ ID NO: 61;
   (ii) a first spacer comprising the nucleotide sequence SEQ ID NO: 1848, or a nucleotide sequence having at least one, two, or three modifications, e.g., substitutions, but no more than four modifications, e.g., substitutions, relative to GATAGTTA; and
   (iii) a second encoded miR122 binding site comprising the nucleotide sequence of SEQ ID NO: 61, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions, but no more than ten modifications, e.g., substitutions, relative to SEQ ID NO: 61; or
   (B) (i) a first encoded miR122 binding site comprising the nucleotide sequence of SEQ ID NO: 61, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions, but no more than ten modifications, e.g., substitutions, relative to SEQ ID NO: 61;
   (ii) a first spacer comprising the nucleotide sequence GATAGTTA, or a nucleotide sequence having at least one, two, or three modifications, e.g., substitutions, but no more than four modifications, e.g., substitutions, relative to GATAGTTA;
   (iii) a second encoded miR122 binding site comprising the nucleotide sequence of SEQ ID NO: 61, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions, but no more than ten modifications, e.g., substitutions, relative to SEQ ID NO: 61;
   (iv) a second spacer comprising the nucleotide sequence of GATAGTTA, or a nucleotide sequence having at least one, two, or three modifications, e.g., substitutions, but no more than four modifications, e.g., substitutions, relative to GATAGTTA; and
   (v) a third encoded miR122 binding site comprising the nucleotide sequence of SEQ ID NO: 61, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions, but no more than ten modifications, e.g., substitutions, relative to SEQ ID NO: 61.
519. The AAV particle of any one of embodiments 469-518, wherein the viral genome comprises an encoded miR183 binding site.
520. The AAV particle of any one of embodiments 469-519, wherein the viral genome comprises at least 1-5 copies, e.g., 1, 2, or 3 copies of a miR183 binding site, optionally wherein each copy is continuous (e.g., not separated by a spacer), or each copy is separated by a spacer, optionally wherein the spacer comprises the nucleotide sequence GATAGTTA, or a nucleotide sequence having at least one, two, or three modifications, e.g., substitutions, but no more than four modifications, e.g., substitutions, relative to GATAGTTA.
521. The AAV particle of embodiment 520, wherein the encoded miR183 binding site comprises the nucleotide sequence of SEQ ID NO: 60, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions, but no more than ten modifications, e.g., substitutions, relative to SEQ ID NO: 60.
522. The AAV particle of any one of embodiments 469-521, wherein the viral genome comprises:
(A) (i) a first encoded miR183 binding site comprising the nucleotide sequence of SEQ ID NO: 60, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions, but no more than ten modifications, e.g., substitutions, relative to SEQ ID NO: 60;
   (ii) a first spacer comprising the nucleotide sequence GATAGTTA, or a nucleotide sequence having at least one, two, or three modifications, e.g., substitutions, but no more than four modifications, e.g., substitutions, relative to GATAGTTA; and
   (iii) a second encoded miR183 binding site comprising the nucleotide sequence of SEQ ID NO: 60, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions, but no more than ten modifications, e.g., substitutions, relative to SEQ ID NO: 60; or
(B) (i) a first encoded miR183 binding site comprising the nucleotide sequence of SEQ ID NO: 60, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions, but no more than ten modifications, e.g., substitutions, relative to SEQ ID NO: 60;
   (ii) a first spacer comprising the nucleotide sequence GATAGTTA, or a nucleotide sequence having at least one, two, or three modifications, e.g., substitutions, but no more than four modifications, e.g., substitutions, relative to GATAGTTA;
   (iii) a second encoded miR183 binding site comprising the nucleotide sequence of SEQ ID NO: 60, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions, but no more than ten modifications, e.g., substitutions, relative to SEQ ID NO: 60;
   (iv) a second spacer comprising the nucleotide sequence of GATAGTTA, or a nucleotide sequence having at least one, two, or three modifications, e.g., substitutions, but no more than four modifications, e.g., substitutions, relative to GATAGTTA; and
   (v) a third encoded miR183 binding site comprising the nucleotide sequence of SEQ ID NO: 60, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions, but no more than ten modifications, e.g., substitutions, relative to SEQ ID NO: 60.
523. The AAV particle of any one of embodiments 469-522, wherein the viral genome comprises an encoded miR 122 binding site and a miR-1 binding site.
524. The AAV particle of any one of embodiments 469-523, wherein the viral genome comprises:
   (i) the nucleotide sequence of any one of SEQ ID NOs: 125, 109, 4820, or 4028-4041; a nucleotide sequence comprising at least 1, 2, 3, 4, 5 but no more than 30, 20, or 10 different nucleotides, relative to any one of SEQ ID NOs: 125, 109, 4820, or 4028-4041; a nucleotide sequence comprising at least 1, 2, 3, 4, 5 but no more than 30, 20, or 10 substitutions relative to the nucleotide sequence of any one of SEQ ID NOs: 125, 109, 4820, or 4028-4041; or a nucleotide sequence with at least 70%, 75%, 80%, 85%, 90%, 95%, 99% or 100% sequence identity to any one of SEQ ID NOs: 125, 109, 4820, or 4028-4041;
   (ii) the nucleotide sequence of SEQ ID NO: 109; a nucleotide sequence comprising at least 1, 2, 3, 4, 5 but no more than 30, 20, or 10 different nucleotides, relative to SEQ ID NO: 109; a nucleotide sequence comprising at least 1, 2, 3, 4, 5 but no more than 30, 20, or 10 substitutions relative to the nucleotide sequence of SEQ ID NO: 109; or a nucleotide sequence with at least 70%, 75%, 80%, 85%, 90%, 95%, 99% or 100% sequence identity to SEQ ID NO: 109;
   (iii) the nucleotide sequence of SEQ ID NO: 125; a nucleotide sequence comprising at least 1, 2, 3, 4, 5 but no more than 30, 20, or 10 different nucleotides, relative to SEQ ID NO: 125; a nucleotide sequence comprising at least 1, 2, 3, 4, 5 but no more than 30, 20, or 10 substitutions relative to the nucleotide sequence of SEQ ID NO: 125; or a nucleotide sequence with at least 70%, 75%, 80%, 85%, 90%, 95%, 99% or 100% sequence identity to SEQ ID NO: 125;
   (iv) the nucleotide sequence of SEQ ID NO: 4820; a nucleotide sequence comprising at least 1, 2, 3, 4, 5 but no more than 30, 20, or 10 different nucleotides, relative to SEQ ID NO: 4820; a nucleotide sequence comprising at least 1, 2, 3, 4, 5 but no more than 30, 20, or 10 substitutions relative to the nucleotide sequence of SEQ ID NO: 4820; or a nucleotide sequence with at least 70%, 75%, 80%, 85%, 90%, 95%, 99% or 100% sequence identity to SEQ ID NO: 4820.
525. The AAV particle of any one of embodiments 469-524, wherein the viral genome comprises, from 5′ to 3′:
   (a) an ITR;
   (b) a filler sequence;
   (b) the promoter;
   (d) the nucleic acid encoding the polynucleotide targeting human SOD1;
   (e) a polyA signal region; and
   (f) an ITR.
526. The AAV particle of any one of embodiments 469-525, wherein the viral genome comprises, from 5′ to 3′:
   (a) an ITR comprising the nucleotide sequence of SEQ ID NO: 126, or a nucleotide sequence at least 90%, 95%, 96%, 97%, 98%, or 99% identical thereto;
   (b) a filler sequence comprising the nucleotide sequence of SEQ ID NO: 82, or a nucleotide sequence at least 90%, 95%, 96%, 97%, 98%, or 99% identical thereto;
   (b) the promoter comprising the nucleotide sequence of SEQ ID NO: 83, or a nucleotide sequence at least 90%, 95%, 96%, 97%, 98%, or 99% identical thereto;
   (d) the nucleic acid encoding the polynucleotide targeting human SOD1 comprising the nucleotide sequence of SEQ ID NO: 2562, or a nucleotide sequence at least 90%, 95%, 96%, 97%, 98%, or 99% identical thereto;
   (e) a polyA signal region comprising the nucleotide sequence of SEQ ID NO: 129, or a nucleotide sequence at least 90%, 95%, 96%, 97%, 98%, or 99% identical thereto; and
   (f) an ITR comprising the nucleotide sequence of SEQ ID NO: 130, or a nucleotide sequence at least 90%, 95%, 96%, 97%, 98%, or 99% identical thereto.
527. The AAV particle of any one of embodiments 469-526, wherein the viral genome comprises:
   (i) the nucleotide sequence of any one of SEQ ID NOs: 109, 125, 4820, or 4028-4041; a nucleotide sequence comprising at least 1, 2, 3, 4, or 5 but no more than 30, 20, or 10 different nucleotides, relative to the nucleotide sequence of any one of SEQ ID NOs: 109, 125, or 4028-4041; or a nucleotide sequence at least 90%, 95%, 99% or 100% identical to any one of SEQ ID NOs: 109, 125, 4820, or 4028-4041;
   (ii) the nucleotide sequence of SEQ ID NO: 109; a nucleotide sequence comprising at least 1, 2, 3, 4, or 5 but no more than 30, 20, or 10 different nucleotides, relative to SEQ ID NO: 109; or a nucleotide sequence at least 90%, 95%, 99% or 100% sequence identity to SEQ ID NO: 109;
   (iii) the nucleotide sequence of SEQ ID NO: 125; a nucleotide sequence comprising at least 1, 2, 3, 4, or 5 but no more than 30, 20, or 10 different nucleotides, relative to SEQ ID NO: 125; or a nucleotide sequence at least 90%, 95%, 99% or 100% sequence identity to SEQ ID NO: 125; or
   (iv) the nucleotide sequence of SEQ ID NO: 4820; a nucleotide sequence comprising at least 1, 2, 3, 4, or 5 but no more than 30, 20, or 10 different nucleotides, relative to SEQ ID NO: 4820; or a nucleotide sequence at least 90%, 95%, 99% or 100% sequence identity to SEQ ID NO: 4820.
528. The AAV particle of any one of embodiments 469-527, wherein the viral genome comprises the nucleotide sequence of SEQ ID NO: 109; a nucleotide sequence comprising at least 1, 2, 3, 4, or 5 but no more than 30, 20, or 10 different nucleotides, relative to SEQ ID NO: 109; or a nucleotide sequence at least 90%, 95%, 99% or 100% sequence identity to SEQ ID NO: 109.
529. An AAV particle comprising:
   (i) an AAV capsid variant comprising the amino acid sequence of SEQ ID NO: 69; and
   (ii) a viral genome comprising the nucleotide sequence of SEQ ID NO: 109.
530. An AAV particle comprising:
   (i) an AAV capsid variant comprising the amino acid sequence of SEQ ID NO: 36; and
   (ii) a viral genome comprising the nucleotide sequence of SEQ ID NO: 109.
531. An AAV particle comprising:
   (i) an AAV capsid variant comprising the amino acid sequence of SEQ ID NO: 982; and
   (ii) a viral genome comprising the nucleotide sequence of SEQ ID NO: 109.
532. The AAV particle of any one of embodiments 469-531, wherein the viral genome is single stranded or self-complementary.
533. The AAV particle of any one of embodiments 469-532, wherein the viral genome further comprises a nucleotide sequence encoding a Rep protein, e.g., a non-structural protein, wherein the Rep protein comprises a Rep78 protein, a Rep68, Rep52 protein, and/or a Rep40 protein.
534. The AAV particle of embodiment 533, wherein the Rep78 protein, the Rep68 protein, the Rep52 protein, and/or the Rep40 protein are encoded by at least one Rep gene.
535. A cell, e.g., a host cell, comprising the AAV particle of any one of the preceding embodiments.
536. The cell of embodiment 535, wherein the cell is a mammalian cell or an insect cell.
537. The cell of embodiment 535 or 536, wherein the cell is a cell of a brain region or a spinal cord region, optionally a cell of the frontal cortex, sensory cortex, motor cortex, caudate, dentate nucleus, cerebellar cortex, cerebral cortex, brain stem, hippocampus, thalamus, putamen, cervical spinal cord region, thoracic spinal cord region, and/or lumbar spinal cord region.
538. The cell of any one of embodiments 535-537, wherein the cell is a neuron (e.g., a NeuN+neuron), a sensory neuron, a motor neuron, a dopaminergic neuron (e.g., a TH+neuron), an astrocyte (e.g., a Sox9+astrocyte), or a glial cell.
539. A method of making the AAV particle of any one of embodiments 1-534, comprising: (i) providing a host cell comprising a viral genome; and
   (ii) incubating the host cell under conditions suitable to enclose the viral genome in an AAV capsid variant, e.g., an AAV capsid variant described herein;
thereby making the AAV particle.
540. The method of embodiment 539, further comprising, prior to step (i), introducing a first nucleic acid molecule comprising the viral genome into the host cell.
541. The method of embodiment 539 or 540, wherein the host cell comprises a second nucleic acid encoding the capsid variant.
542. The method of embodiment 541, wherein the second nucleic acid molecule is introduced into the host cell prior to, concurrently with, or after the first nucleic acid molecule.
543. A pharmaceutical composition comprising the AAV particle of any one of embodiments 1-534, and a pharmaceutically acceptable excipient.
544. A method of delivering a payload to a cell or tissue (e.g., a CNS cell or a CNS tissue), comprising administering an effective amount of the pharmaceutical composition of embodiment 543, or the AAV particle of any one of embodiments 1-534.
545. The method of embodiment 544, wherein the cell is a cell a cell of a brain region or a spinal cord region, optionally a cell of the frontal cortex, sensory cortex, motor cortex, caudate, dentate nucleus, cerebellar cortex, cerebral cortex, brain stem, hippocampus, thalamus, putamen, cervical spinal cord region, thoracic spinal cord region, and/or lumbar spinal cord region.
546. The method of embodiment 544 or 545, wherein the cell is a neuron (e.g., a NeuN+neuron or dopaminergic neuron (e.g., a TH+positive neuron)), a sensory neuron, a motor neuron, or an astrocyte (e.g., a Sox9+astrocyte).
547. The method of any one of embodiments 544-546, wherein the cell or tissue is within a subject.
548. The method of embodiment 547, wherein the subject has, has been diagnosed with having, or is at risk of having a neurological, e.g., a neurodegenerative disorder.
549. The method of embodiment 547 or 548, wherein the subject has, has been diagnosed with having, or is at risk of having a disease associated with SOD1 expression or activity.
550. The method of any one of embodiments 547-549, wherein the subject has, has been diagnosed with having, or is at risk of having a disease associated with SOD1 expression or activity.
551. The method of any one of embodiments 547-550, wherein the subject has, has been diagnosed with having, or is at risk of having amyotrophic lateral sclerosis.
552. A method of treating a subject having or diagnosed with having a neurological disorder, e.g., a neurodegenerative disorder, comprising administering to the subject an effective amount of the pharmaceutical composition of embodiment 543, or the AAV particle of any one of embodiments 1-534.
553. A method of treating a subject having or diagnosed with having a disease related to expression of SOD1, comprising administering to the subject an effective amount of the pharmaceutical composition of embodiment 543, or the AAV particle of any one of embodiments 1-534.
554. A method of treating a subject having or diagnosed with a disease associated with SOD1 expression or activity, comprising administering to the subject an effective amount of the pharmaceutical composition of embodiment 543, or the AAV particle of any one of embodiments 1-534.
555. The method of any one of embodiments 548-554, wherein the neurological disorder, neurodegenerative disorder, or disease associated with SOD1 expression comprises amyotrophic lateral sclerosis (ALS).
556. The method embodiment 555, wherein the ALS is:
   (i) familial ALS;
   (ii) sporadic ALS;
   (iii) early stage ALS;
   (iv) middle stage ALS; and/or
   (v) late stage ALS.
557. The method of any one of embodiments 552-556, where treating comprises prevention of progression of the disease or disorder in the subject.
558. The method of any one of embodiments 547-557, wherein the subject is a human.
559. The method of any one of embodiments 555-558, wherein treatment comprises amelioration of a symptom of ALS in the subject.
560. The method of embodiment 559, wherein the symptom comprises motor neuron degeneration, muscle weakness, stiffness of muscles, muscle atrophy, muscle stiffness, fasciculation development, frontotemporal dementia, slurred speech, difficulty breathing, or a combination thereof.
561. The method of any one of embodiments 547-560, wherein the AAV particle or pharmaceutical composition is administered to the subject intravenously, via intra-cisterna magna injection (ICM), intracerebrally, intrathecally, intracerebroventricularly, via intraparenchymal administration, or intramuscularly.
562. The method of any one of embodiments 547-561, wherein the AAV particle or pharmaceutical composition is administered to the subject via focused ultrasound (FUS), e.g., coupled with the intravenous administration of microbubbles (FUS-MB), or MRI-guided FUS coupled with intravenous administration.
563. The method of any one of embodiments 547-561, wherein the AAV particle or pharmaceutical composition is administered to the subject intravenously.
564. The method of any one of embodiments 544-563, wherein administration of the AAV particle or pharmaceutical composition results in a decreased presence, level, and/or activity of a gene, mRNA, protein, or combination thereof.
565. The method of any one of embodiments 544-564, wherein administration of the AAV particle or pharmaceutical composition results in at least a 30-60% reduction of SOD1 mRNA expression, e.g., in the spinal cord of the subject, relative to a subject that has not received the AAV particle or pharmaceutical composition 566. The method of any one of embodiments 544-563, wherein administration of the AAV particle or pharmaceutical composition results in an increased presence, level, and/or activity of a gene, mRNA, protein, or a combination thereof.
567. The pharmaceutical composition of embodiment 543, or the AAV particle of any one of embodiments 1-534, for use in a method of delivering a payload to a cell or tissue.
568. The pharmaceutical composition of embodiment 543, or the AAV particle of any one of embodiments 1-534, for use in a method of treating a neurological disorder, a neurodegenerative disorder, a disease associated with SOD1 expression or activity.
569. The pharmaceutical composition of embodiment 543, or the AAV particle of any one of embodiments 1-534, for use in the manufacture of a medicament.
570. Use of the pharmaceutical composition of embodiment 543, or the AAV particle of any one of embodiments 1-534, in the manufacture of a medicament.
571. Use of the pharmaceutical composition of embodiment 543, or the AAV particle of any one of embodiments 1-534, in the manufacture of a medicament for treating a neurological disorder, a neurodegenerative disorder, a disease associated with SOD1 expression or activity.

The details of various aspects or embodiments of the present disclosure are set forth below. Other features, objects, and advantages of the disclosure will be apparent from the description and the claims. In the description, the singular forms also include the plural unless the context clearly dictates otherwise. Unless defined otherwise, all technical and scientific terms used herein have the same meaning as commonly understood by one of ordinary skill in the art in the field of this disclosure. In the case of conflict, the present description will control.

### BRIEF DESCRIPTION OF THE DRA WINGS

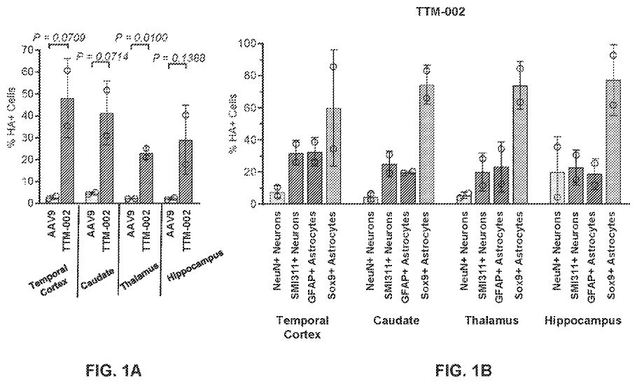

FIG. 1A is a graph showing quantification of the percentage of transduced cells having HA+nuclei as measured by co-localization of nuclear H2B-HA staining and hematoxylin (% HA+ cells) in the indicated brain regions (temporal cortex, caudate, thalamus, or hippocampus) of African green monkeys at day 28 post-intravenous injection of AAV particles comprising the TTM-002 capsid variant or the AAV9 capsid control and a self-complementary genome encoding a histone 2B protein with an HA-tag at a dose of 1e13 VG/kg. FIG. 1B is a graph showing the percentage of HA+ cells among cells positive for the indicated marker (NeuN+neurons, SMI311+Neurons, GFAP+astrocytes, or Sox9+astrocytes) in the indicated brain regions (temporal cortex, caudate, thalamus, or hippocampus) of African green monkeys at day 28 post-intravenous injection of AAV particles comprising the TTM-002 capsid variant and a self-complementary genome encoding a histone 2B protein with an HA-tag at a dose of 1e13 VG/kg. Plotted data in FIGS. 1A-1B represent one slice per monkey (n=2). Quantitative image analysis was performed on 1e3 to 1e5 cells according to region size. All P values are derived from an unpaired two-tailed t-test.

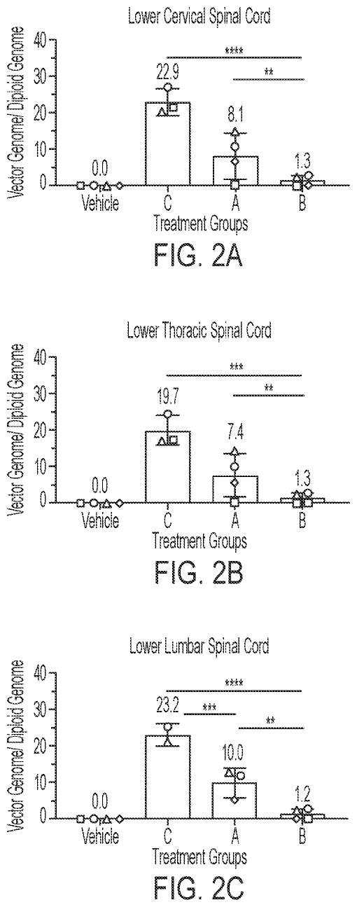

FIGS. 2A-2C are bar graphs depicting the distribution of an AAV particle with a VOY101 capsid and a viral genome (SEQ ID NO: 109, Table 15) encoding the SOD1 targeting modulatory polynucleotide, miR104-788.2 (Table 14), under the control of an H1 promoter (AAV_VOY101.SOD1) in the lower cervical spinal cord (FIG. 2A), lower thoracic spinal cord (FIG. 2B), and lower lumbar spinal cord (FIG. 2C) of SOD1^{G93A} transgenic mice. Groupings were as follows: Vehicle (Group V), 2E13 vg/kg (Group C), 6.3E12 (Group A), and 2E12 (Group B). ** p<0.01; *** p<0.001; **** p<0.0001 (1-way ANOVA and Tukey's multiple comparisons).

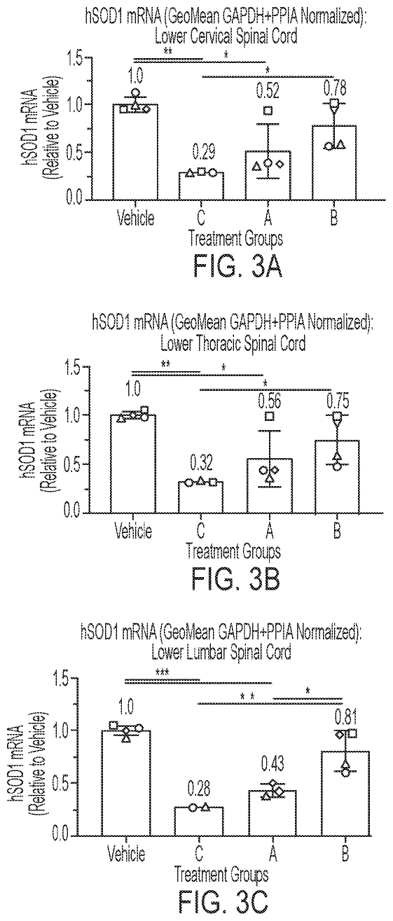

FIGS. 3A-3C are bar graphs depicting the reduction in human SOD1 (hSOD1) mRNA after intravenous administration of an SOD1 miRNA-encoding AAV particle with a VOY101 capsid and a viral genome (SEQ ID NO: 109, Table 15) encoding the SOD1 targeting modulatory polynucleotide miR104-788.2 (SEQ ID NO: 2562, Table 14) under the control of an H1 promoter (AAV_VOY101.SOD1) in the lower cervical spinal cord (FIG. 3A), lower thoracic spinal cord (FIG. 3B), and lower lumbar spinal cord (FIG. 3C) in SOD1^{G93A} transgenic mice. Groupings were as follows: Vehicle (Group B), 2E13 vg/kg (Group C), 6.3E12 (Group D), and 2E12 (Group E). *p<0.05; ** p<0.01; *** p<0.001 (1-way ANOVA and Tukey's multiple comparisons). hSOD1 mRNA levels are normalized to GAPDH+PPIA, and results are presented as values relative to the vehicle group (Group B).

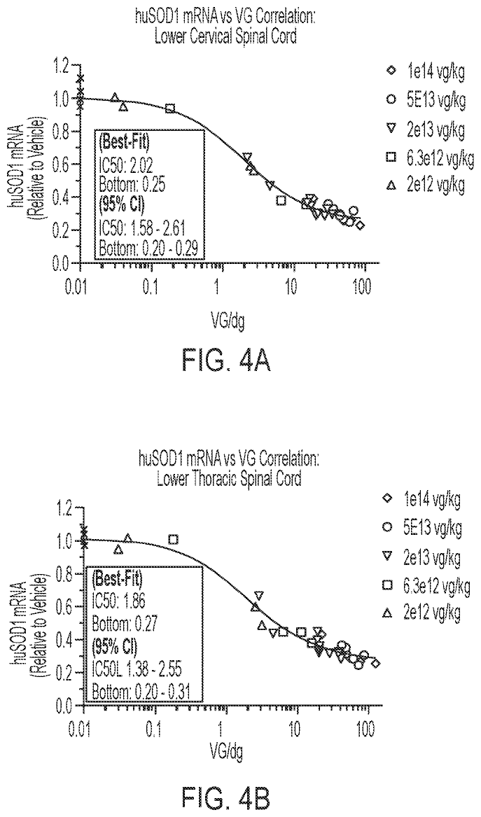

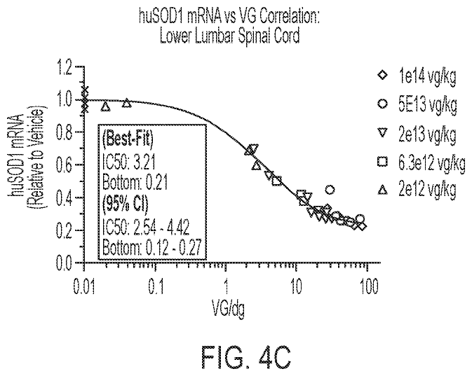

FIGS. 4A-4C are graphs depicting correlations between hSOD1 mRNA levels and AAV particle distribution (VG/dg) in the lower cervical spinal cord (FIG. 4A), lower thoracic spinal cord (FIG. 4B), and lower lumbar spinal cord (FIG. 4C) of SOD 1^{G93A} transgenic mice.

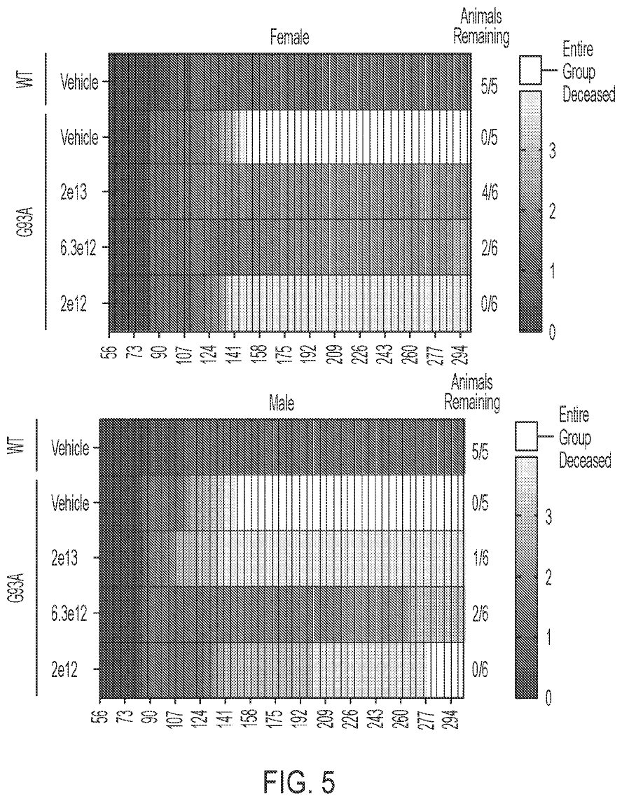

FIG. 5 shows the Neuroscore composite ranking of female and male wild-type mice administered vehicle and SOD1^{G93A} mice administered vehicle, 2E13 vg/kg, 6.3E12, or 2E12 vg/kg SOD1 miRNA AAV particle. The number of animals remaining in each group is also presented.

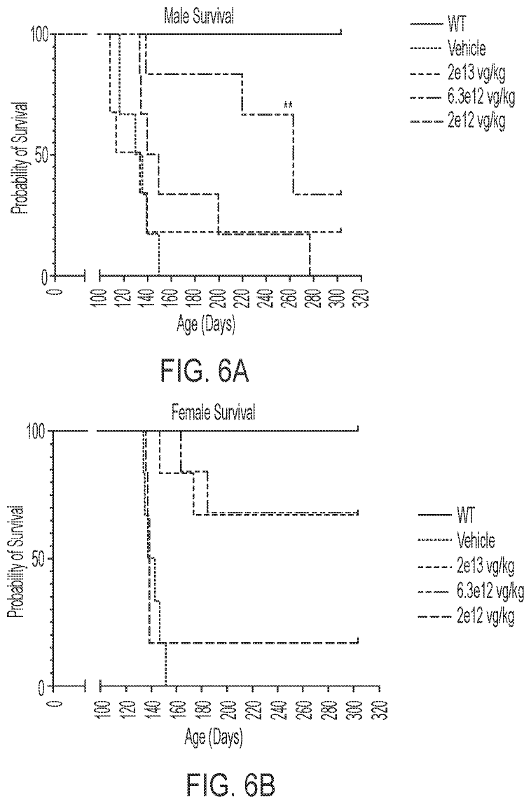

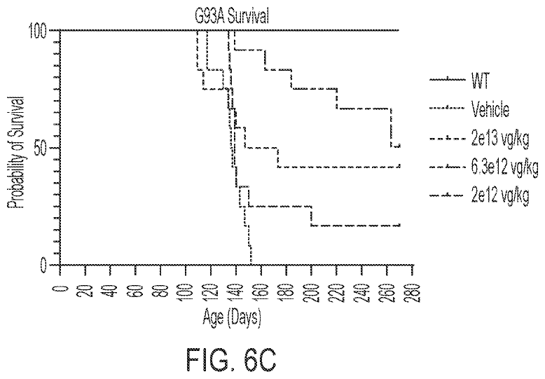

FIGS. 6A-6C are graphs depicting Kaplan-Meier survival curves for female (FIG. 6A), male (FIG. 6B), and all (FIG. 6C) wild-type mice administered vehicle and SOD1^{G93A} mice administered vehicle, 2E13 vg/kg, 6.3E12, or 2E12 vg/kg SOD1 miRNA AAV particle. ** p=0.003; log-rank (Mantel-Cox) test.

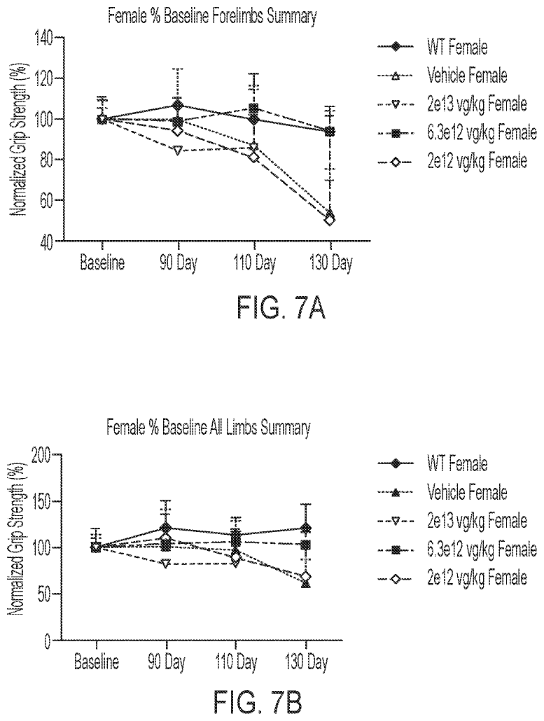

FIGS. 7A and 7B are graphs depicting grip strength normalized to baseline for forelimbs (FIG. 7A) and all limbs combined (FIG. 7B) in female wild-type mice administered vehicle and SOD1^{G93A} mice administered vehicle, 2E13 vg/kg, 6.3E12, or 2E12 vg/kg SOD1 miRNA AAV particle. Baseline corresponds to the week prior to intravenous SOD1 miRNA AAV particle administration.

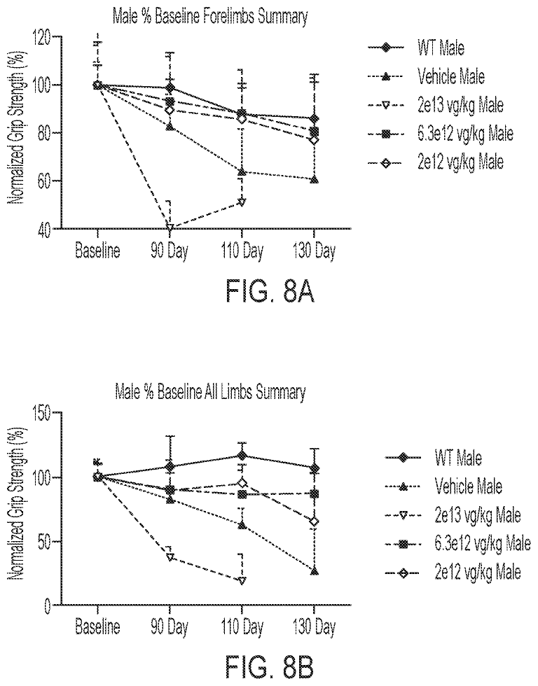

FIGS. 8A and 8B are graphs depicting grip strength normalized to baseline for forelimbs (FIG. 8A) and all limbs combined (FIG. 8B) in male wild-type mice administered vehicle and SOD1^{G93A} mice administered vehicle, 2E13 vg/kg, 6.3E12, or 2E12 vg/kg SOD1 miRNA AAV particle. Baseline corresponds to the week prior to intravenous SOD1 miRNA AAV particle administration.

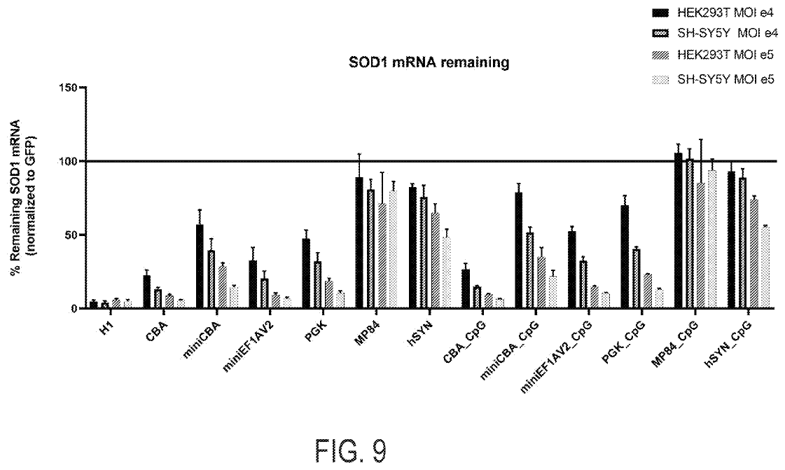

FIG. 9 is a graph depicting the percentage of remaining SOD1 mRNA in cell lines after treatment with SOD1 targeting modulatory polynucleotide miR104-788.2 under the control of various promoters.

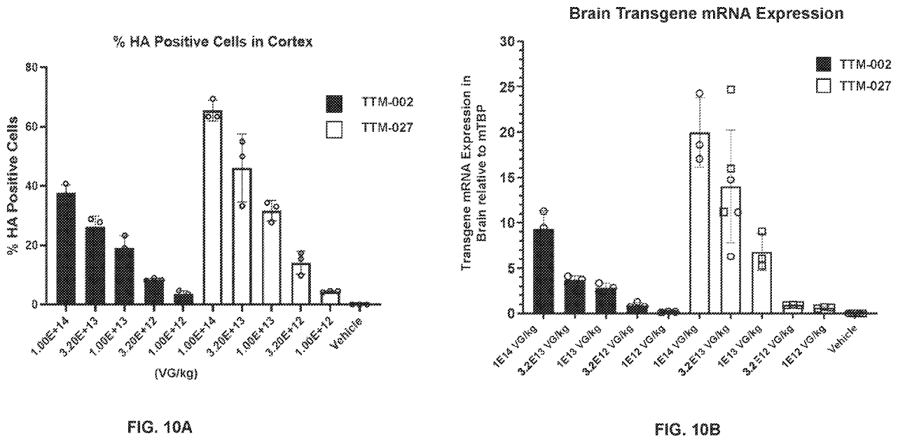

FIG. 10A is a graph showing the percentage of HA positive cells (percent of cells transduced by the indicated capsid variant) in the cortex in mice on the Y axis at the indicated doses on the X-axis (from highest to lowest dose: 1e14 vg/kg, 3.2e13 vg/kg, 1e13 vg/kg, 3.2e12 vg/kg, or 1e12 vg/kg) at 28 days post-intravenous administration of AAV particles comprising the TTM-002 or TTM-027 AAV capsid variant. FIG. 10B is a graph showing the mRNA transgene expression relative to the housekeeping gene in the brain of the mice on the Y axis at the indicated doses on the X-axis (from highest to lowest dose: 1e14 vg/kg, 3.2e13 vg/kg, 1e13 vg/kg, 3.2e12 vg/kg, or 1e12 vg/kg) at 28 days post-intravenous administration of AAV particles comprising the TTM-002 or TTM-027 AAV capsid variant.

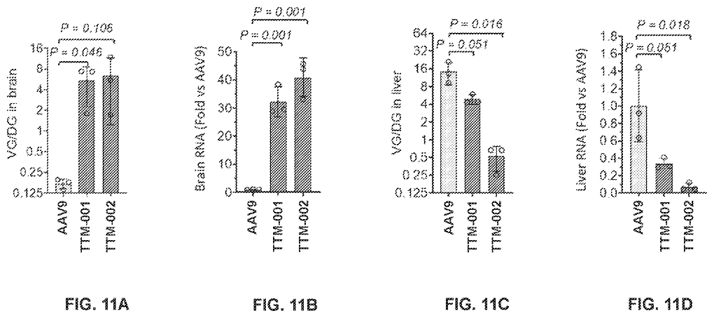

FIGS. 11A-11D are a series of graphs demonstrating tropism of TTM-001 and TTM-002 relative to the AAV9 control in the brain and liver at 28 days post-intravenous administration in mice at a dose of 1e13 VG/kg. FIG. 11A shows the viral genomes (VG)/diploid genomes (DG) in the brain for the AAV9 control, TTM-001, or TTM-002; FIG. 11B shows the brain RNA (fold vs AAV9) for the AAV9 control, TTM-001, or TTM-002; FIG. 11C shows the VG/DG in the liver for the AAV9 control, TTM-001, or TTM-002; and FIG. 11D shows the liver RNA (fold vs AAV9) for the AAV9 control, TTM-001, or TTM-002. Each data point represents an individual mouse and all plotted values represent mean±SD (n=3). P values are derived from an unpaired two-tailed t-test.

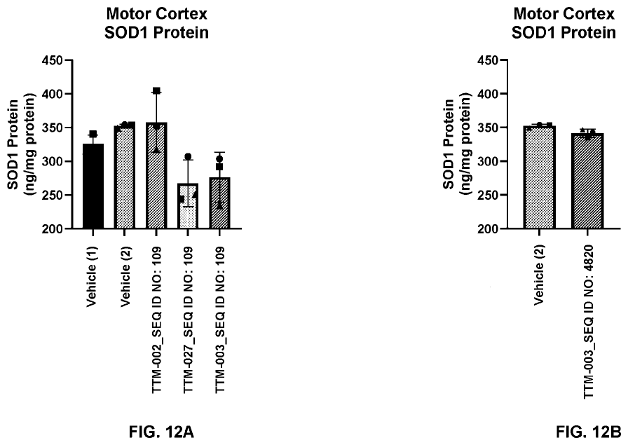

FIG. 12A is a graph showing the SOD1 protein measured in the motor cortex on day 56 post-intravenous administration of the vehicle control AAV particles comprising the capsid and viral genome combination indicated on the X-axis, from left to right: vehicle (1); vehicle (2); AAV particles comprising the TTM-002 capsid variant encapsulating the viral genome comprising SEQ ID NO: 109 (TTM-002_SEQ ID NO: 109); AAV particles comprising the TTM-027 capsid variant encapsulating the viral genome comprising SEQ ID NO: 109 (TTM-027_SEQ ID NO: 109); or AAV particles comprising the TTM-003 capsid variant encapsulating the viral genome comprising SEQ ID NO: 109 (TTM-003_SEQ ID NO: 109). FIG. 12B is a graph showing the SOD1 protein measured in the motor cortex on day 56 post-intravenous administration of the vehicle control AAV particles comprising the capsid and viral genome combination indicated on the X-axis, from left to right: vehicle (2) and AAV particles comprising the TTM-003 capsid variant encapsulating the viral genome comprising SEQ ID NO: 4820 (TTM-003_SEQ ID NO: 4820).

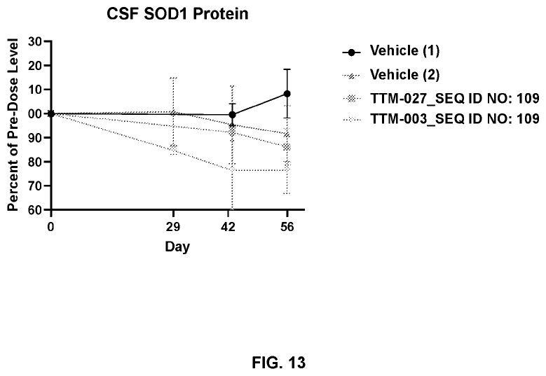

FIG. 13 is a graph showing SOD1 protein levels as a percent of pre-dose SOD1 protein levels (percent of pre-dose level) in the CSF on days 29, 42, and 56 post-intravenous injection of vehicle (1); vehicle (2); AAV particles comprising the TTM-027 capsid variant encapsulating the viral genome comprising SEQ ID NO: 109 (TTM-027_SEQ ID NO: 109); or AAV particles comprising the TTM-003 capsid variant encapsulating the viral genome comprising SEQ ID NO: 109 (TTM-003_SEQ ID NO: 109).

### DETAILED DESCRIPTION

### I. Overview

Disorders Associated with the Spinal Cord

The spinal cord is one of two components that together characterize the central nervous system (CNS; brain and spinal cord). The spinal cord connects the body to the brain, serving as a conduit for the messages and communications necessary for movement and sensation. The spinal cord is a fragile, thin, tubular bundle made up of nerve fibers and cell bodies, as well as support cells, housed within the vertebral column.

The motor neurons and pathways of the spinal cord are important for the initiation, execution, modification, and precision of movement. When these neurons and/or pathways are damaged in some manner, such as, but not limited to, trauma, tumorous growth, cardiovascular defects, inflammation, de-myelination, neuropathy, degeneration and/or cell death, the consequence is typically a defect in some form of movement. Similarly, sensory neurons and pathways of the spinal cord are critical for proprioception and sensation, and when damaged, can result in an inability to sense certain stimuli and/or pain syndromes.

Non-limiting examples of disorders such as those described above, which are associated with the spinal cord include, but are not limited to, motor neuron disease, amyotrophic lateral sclerosis (ALS; Lou Gehrig's disease), progressive bulbar palsy, pseudobulbar palsy, primary lateral sclerosis, progressive muscular atrophy, spinal muscular atrophy, post-polio syndrome, bulbar palsy, Kennedy's disease, hereditary spastic paraplegia, Friedreich's ataxia, Charcot-Marie-Tooth disease, hereditary motor and sensory neuropathy, peroneal muscular atrophy, neuropathies, de-myelinating diseases, viral de-myelination, metabolic de-myelination, multiple sclerosis, neuromyelitis optica (Devic's disease), concentric sclerosis (Baló's sclerosis), ataxias, paraplegia, spinocerebellar ataxia, acute-disseminated encephalomyelitis, complex regional pain syndrome (CPRS I and CPRS II), ataxia telangiectasia, episodic ataxia, multiple system atrophy, sporadic ataxia, lipid storage diseases, Niemann-Pick disease, Fabry disease, Faber's disease, GM1 or GM2 gangliosidoses, Tay-Sachs disease, Sandhoff disease, Krabbe disease, metachromatic leukodystrophy, Machado-Joseph disease (spinocerebellar ataxia type 3), meningitis, myelitis, myopathy, mitochondrial myopathy, encephalomyopathy, Barth syndrome, Chronic progressive external ophtalmoplegia, Kearns-Sayre syndrome, Leigh syndrome, mitochondrial DNA depletion syndromes, myoclonus epilepsy with ragged red fibers, NARP (neuropathy, ataxia and retinitis pigmentosa, diseases of the neuromuscular junction, myasthenia gravis, myoclonus, neuropathic pain, neurodegenerative diseases, Parkinson's disease, Alzheimer's disease, Huntington's disease, Lewy body disease, Vitamin B12 deficiency, subacute combined degeneration of the spinal cord (Lichtheim's disease), tropical spastic paraparesis, distal hereditary motor neuronopathies, Morvan's syndrome, leukodystrophies, and/or Rett syndrome.

In some embodiments, the compositions and methods of the present disclosure may be used to treat any disease of the central nervous system.

In some embodiments, the compositions and methods of the present disclosure may be used to treat a disease associated with the spinal cord.

In some embodiments, the compositions and methods of the present disclosure may be used for the treatment of a neurodegenerative disease.

In some embodiments, the compositions and methods of the present disclosure may be used for the treatment of a motor neuron disease.

In some embodiments, the compositions and methods of the present disclosure may be used for the treatment of amyotrophic lateral sclerosis (ALS).

Amyotrophic lateral sclerosis (ALS) and SOD1

Amyotrophic lateral sclerosis (ALS), an adult-onset neurodegenerative disorder, is a progressive and fatal disease characterized by the selective death of motor neurons in the motor cortex, brainstem and spinal cord. Patients diagnosed with ALS develop a progressive muscle phenotype characterized by spasticity, hyperreflexia or hyporeflexia, fasciculations, muscle atrophy and paralysis. These motor impairments are caused by the de-innervation of muscles due to the loss of motor neurons. The major pathological features of ALS include degeneration of the corticospinal tracts and extensive loss of lower motor neurons (LMNs) or anterior horn cells (Ghatak et al., J Neuropathol Exp Neurol., 1986, 45, 385-395), degeneration and loss of Betz cells and other pyramidal cells in the primary motor cortex (Udaka et al., Acta Neuropathol, 1986, 70, 289-295; Maekawa et al., Brain, 2004, 127, 1237-1251) and reactive gliosis in the motor cortex and spinal cord (Kawamata et al., Am J Pathol., 1992, 140,691-707; and Schiffer et al., J Neurol Sci., 1996, 139, 27-33). ALS is usually fatal within 3 to 5 years after the diagnosis due to respiratory defects and/or inflammation (Rowland L P and Shneibder N A, N Engl. J. Med., 2001, 344, 1688-1700).

A cellular hallmark of ALS is the presence of proteinaceous, ubiquitinated, cytoplasmic inclusions in degenerating motor neurons and surrounding cells (e.g., astrocytes). Ubiquitinated inclusions (i.e., Lewy body-like inclusions or Skein-like inclusions) are the most common and specific type of inclusion in ALS and are found in LMNs of the spinal cord and brainstem, and in corticospinal upper motor neurons (UMNs) (Matsumoto et al., J Neurol Sci., 1993, 115, 208-213; and Sasak and Maruyama, Acta Neuropathol., 1994, 87, 578-585). A few proteins have been identified to be components of the inclusions, including ubiquitin, Cu/Zn superoxide dismutase 1 (SOD1), peripherin and Dorfin. Neurofilamentous inclusions are often found in hyaline conglomerate inclusions (HCIs) and axonal ‘spheroids’ in spinal cord motor neurons in ALS. Other types and less specific inclusions include Bunina bodies (cystatin C-containing inclusions) and Crescent shaped inclusions (SCIs) in upper layers of the cortex. Other neuropathological features seen in ALS include fragmentation of the Golgi apparatus, mitochondrial vacuolization and ultrastructural abnormalities of synaptic terminals (Fujita et al., Acta Neuropathol. 2002, 103, 243-247).

In addition, in frontotemporal dementia ALS (FTD-ALS), cortical atrophy (including the frontal and temporal lobes) is also observed, which may cause cognitive impairment in FTD-ALS patients.

ALS is a complex and multifactorial disease and multiple mechanisms hypothesized as responsible for ALS pathogenesis include dysfunction of protein degradation, glutamate excitotoxicity, mitochondrial dysfunction, apoptosis, oxidative stress, inflammation, protein misfolding and aggregation, aberrant RNA metabolism, and altered gene expression.

About 10% of ALS cases have family history of the disease, and these patients are referred to as familial ALS (fALS) or inherited patients, commonly with a Mendelian dominant mode of inheritance and high penetrance. The remainder (approximately 90%-95%) is classified as sporadic ALS (sALS), as they are not associated with a documented family history, which is thought to be due to other risk factors, including environmental factors, genetic polymorphisms, somatic mutations, and possibly gene-environmental interactions. In most cases, familial (or inherited) ALS is inherited as autosomal dominant disease, but pedigrees with autosomal recessive and X-linked inheritance and incomplete penetrance exist. Sporadic and familial forms are clinically indistinguishable, suggesting a common pathogenesis. The precise cause of the selective death of motor neurons in ALS remains elusive. Progress in understanding the genetic factors in fALS may shed light on both forms of the disease.

Recently, an explosion in research and understanding of genetic causes of ALS has led to the discovery of mutations in more than 10 different genes now known to cause fALS. The most common ones are found in the genes encoding Cu/Zn superoxide dismutase 1 (SOD1; ~ 20%) (Rosen D R et al., Nature, 1993, 362, 59-62), fused in sarcoma/translated in liposarcoma (FUS/TLS; 1-5%) and TDP-43 (TARDBP; 1-5%). Recently, a hexanucleotide repeat expansion (GGGGCC) n in the C9orf72 gene was identified as the most frequent cause of fALS (~ 40%) in the Western population (reviewed by Renton et al., Nat. Neurosci., 2014, 17, 17-23). Other genes mutated in ALS include alsin (ALS2), senataxin (SETX), vesicle-associated membrane protein (VAPB), angiogenin (ANG). fALS genes control different cellular mechanisms, suggesting that the pathogenesis of ALS is complicated and may be related to several different processes finally leading to motor neuron degeneration.

SOD1 is one of the three human superoxide dismutases identified and characterized in mammals: copper-zinc superoxide dismutase (Cu/ZnSOD or SOD1), manganese superoxide dismutase (MnSOD or SOD2), and extracellular superoxide dismutase (ECSOD or SOD3). SOD1 is a 32 kDa homodimer of a 153-residue polypeptide with one copper- and one zinc-binding site per subunit, which is encoded by SOD1 gene (GeneBank access No.: NM_000454.4) on human chromosome 21 (see Table 19). SOD1 catalyzes the reaction of superoxide anion (02-) into molecular oxygen (O₂) and hydrogen peroxide (H₂O₂) at a bound copper ion. The intracellular concentration of SOD1 is high (ranging from 10 to 100 μM), accounting for 1% of the total protein content in the central nervous system (CNS). The protein is localized not only in the cytoplasm but also in the nucleus, lysosomes, peroxisomes, and mitochondrial intermembrane spaces in eukaryotic cells (Lindenau J et al., Glia, 2000, 29, 25-34). Without wishing to be bound by theory, it is believed in some that in SOD1-ALS, mutation of superoxide dismutase 1 (SOD1) leads to the formation of insoluble SOD1 aggregates in motor neurons and to neurodegeneration.

Mutations in SOD1 gene are carried by 15-20% of fALS patients and by 1-2% of all ALS cases. Currently, at least 170 different mutations distributed throughout the 153-amino acid SOD1 polypeptide have been found to cause ALS, and an updated list can be found at the ALS online Genetic Database (ALSOD) (Wroe R et al., Amyotroph Lateral Scler., 2008, 9, 249-250). Table 53 lists some examples of mutations in SOD1 in ALS. These mutations are predominantly single amino acid substitutions (i.e. missense mutations) although deletions, insertions, and C-terminal truncations also occur. Different SOD1 mutations display different geographic distribution patterns. For instance, about half of all Americans with ALS caused by SOD1 gene mutations have a particular mutation Ala4Val (or A4V). The A4V mutation is typically associated with more severe signs and symptoms. The I113T mutation is by far the most common mutation in the United Kingdom. The most prevalent mutation in Europe is D90A substitution.

TABLE 53
Examples of SOD1 mutations in ALS

| | Mutations |
|---|---|
| Exon1 (220 bp) | Q22L; E21K, G; F20C; N19S; G16A, S; V14M, S; G12R; |
| | G10G, V, R; L8Q, V; V7E; C6G, F; V5L; A4T, V, S |
| Exon2 (97 bp) | T54R; E49K; H48R, Q; V47F, A; H46R; F45C; H43R; G41S, |
| | D; G37R; V29, insA |
| Exon3 (70 bp) | D76Y, V; G72S, C; L67R; P66A; N65S; S59I, S |
| Exon4 (118 bp) | D124G, V; V118L, InsAAAAC; L117V; T116T; R115G; |
| | G114A; I113T, F; I112M, T; G108V; L106V, F; S106L, |
| | delTCACTC; I104F; D101G, Y, H, N; E100G, K; I99V; |
| | V97L, M; D96N, V; A95T, V; G93S, V, A, C, R, D; D90V, |
| | A; A89T, V; T88delACTGCTGAC; V87A, M; N86I, S, D, |
| | K; G85R, S; L84V, F; H80R |
| Exon5 (461 bp) | I151T, S; I149T; V148I, G; G147D, R; C146R, stop; A145T, |
| | G; L144F, S; G141E, stop; A140A, G; N139D, K, H, N; |
| | G138E; T137R; S134N; E133V, delGAA, insTT; E132insTT; |
| | G127R, InsTGGG; L126S, delITT, stop; D126, delTT |

To investigate the mechanism of neuronal death associated with SOD1 gene defects, several rodent models of SOD1-linked ALS were developed in the art, which express the human SOD1 gene with different mutations, including missense mutations, small deletions or insertions. Some examples of ALS mouse models include SOD1G93A, SOD1A4V, SOD1G37R, SOD1G85R, SOD 1D90A, SOD1L84V, SOD11113T, SOD1H36R/H48Q, SOD1G127X, SOD1L126X and SOD1L126deITT There are two transgene rat models carrying two different human SOD1 mutations: SOD1H46R and SOD1G93R. These rodent ALS models can develop muscle weakness similar to human ALS patients and other pathogenic features that reflect several characteristics of the human disease, in particular, the selective death of spinal motor neurons, aggregation of protein inclusions in motor neurons and microglial activation. It is well known in the art that the transgenic rodents are good models of human SOD1-assocaited ALS disease and provide models for studying disease pathogenesis and developing disease treatment.

Studies in animal and cellular models showed that SOD1 pathogenic variants cause ALS by gain of function. That is to say, the superoxide dismutase enzyme gains new but harmful properties when altered by SOD1 mutations. For example, some SOD1 mutated variants in ALS increase oxidative stress (e.g., increased accumulation of toxic superoxide radicals) by disrupting redox cycle. Other studies also indicate that some SOD1 mutated variants in ALS might acquire toxic properties that are independent of its normal physiological function (such as abnormal aggregation of misfolded SOD1 variants). In the aberrant redox chemistry model, mutant SOD1 is unstable and through aberrant chemistry interacts with nonconventional substrates causing reactive oxygen species (ROS) overproduction. In the protein toxicity model, unstable, misfolded SOD1 aggregates into cytoplasmic inclusion bodies, sequestering proteins crucial for cellular processes. These two hypotheses are not mutually exclusive. It has been shown that oxidation of selected histidine residues that bind metals in the active site mediates SOD1 aggregation.

The aggregated mutant SOD1 protein may also induce mitochondrial dysfunction (Vehvilainen P et al., Front Cell Neurosci., 2014, 8, 126), impairment of axonal transport, aberrant RNA metabolism, glial cell pathology and glutamate excitotoxicity. In some sporadic ALS cases, misfolded wild-type SOD1 protein is found in diseased motor neurons which forms “toxic conformation” that is similar to familial ALS-linked SOD1 variants (Rotunno M S and Bosco D A, Front Cell Neurosci., 2013, 16, 7, 253). Such evidence suggests that ALS is a protein misfolding disease analogous to other neurodegenerative diseases such as Alzheimer's disease and Parkinson's disease.

Currently, no curative treatments are available for patients suffering from ALS. Until recently, the only FDA approved drug was Riluzole (also called Rilutek), an inhibitor of glutamate release, with a moderate effect on ALS, only extending survival by 2-3 months if it is taken for 18 months. Unfortunately, patients taking riluzole do not experience any slowing in disease progression or improvement in muscle function. Therefore, riluzole does not present a cure, or even an effective treatment. In 2017, the FDA approved Radicava (edaravone) for the treatment of ALS, the first such approval in 22 years. Administered intravenously and serving as a free-radical scavenger and anti-oxidant, Radicava has been shown to slow disease progression. In a clinical Phase 3 trial (NCT01492686) of 137 patients, Radicava slowed the decline in physical function as compared to those patients taking placebo and as determined by score on the ALS Functional Rating Scale-Revised (ALSFRS-R) (Writing group; Edaravone (MCI-186) ALS 19 Study Group Lancet Neurol. 2017 July;16 (7): 505-512). The approval of Radicava is considered an advance in terms of treatment of ALS, however it is still not a cure. Researchers continue to search for better therapeutic agents.

One approach to inhibit abnormal SOD1 protein aggregation is to silence/inhibit SOD1 gene expression in ALS. It has been reported that small interfering RNAs for specific gene silencing of the mutated allele is therapeutically beneficial for the treatment of fALS (e.g., Ralgh G S et al., Nat. Medicine, 2005, 11 (4), 429-433; and Raoul C et al., Nat. Medicine, 2005, 11 (4), 423-428; and Maxwell M M et al., PNAS, 2004, 101 (9), 3178-3183; and Ding H et al., Chinese Medical J., 2011, 124 (1), 106-110; and Scharz D S et al., Plos Genet., 2006, 2 (9), e140; the content of each of which is incorporated herein by reference in their entirety).

Many other RNA therapeutic agents that target SOD1 gene and modulate SOD1 expression in ALS are taught in the art, such RNA based agents include antisense oligonucleotides and double stranded small interfering RNAs. See, e.g., Wang H et al., J Biol. Chem., 2008, 283 (23), 15845-15852); U.S. Pat. Nos. 7,498,316; 7,632,938; 7,678,895; 7,951,784; 7,977,314; 8,183,219; 8,309,533 and 8, 586, 554; and U.S. Patent publication Nos. 2006/0229268 and 2011/0263680; the content of each of which is herein incorporated by reference in their entirety.

The present disclosure employs viral vectors such as adeno-associated viral (AAV) vectors to deliver siRNAs or SOD1 targeting polynucleotides into cells with high efficiency. The AAV vectors comprising RNAi molecules, e.g., siRNA molecules of the present disclosure may increase the delivery of active agents into motor neurons. SOD1 targeting polynucleotides may be able to inhibit SOD1 gene expression (e.g., mRNA level) significantly inside cells; therefore, ameliorating SOD1 expression induced stress inside the cells such as aggregation of protein and formation of inclusions, increased free radicals, mitochondrial dysfunction and RNA metabolism.

Such SOD1 targeting polynucleotides may be used for treating ALS. According to the present disclosure, methods for treating and/or ameliorating ALS in a patient comprises administering to the patient an effective amount of at least one SOD1 targeting polynucleotide encoding one or more siRNAs into cells and allowing the inhibition/silence of SOD1 gene expression, are provided.

### Adeno-Associated Viral (AAV) Particles

Described herein, inter alia, are compositions comprising isolated, e.g., recombinant, viral particles, e.g., AAV particles, for delivery, e.g., vectorized delivery, of a polynucleotide, e.g., a SOD1 targeting siRNA, and methods of making and using the same. Adeno-associated viruses (AAV) are small non-enveloped icosahedral capsid viruses of the Parvoviridae family characterized by a single stranded DNA viral genome. Parvoviridae family viruses consist of two subfamilies: Parvovirinae, which infect vertebrates, and Densovirinae, which infect invertebrates. The Parvoviridae family includes the Dependovirus genus which includes AAV, capable of replication in vertebrate hosts including, but not limited to, human, primate, bovine, canine, equine, and ovine species.

The parvoviruses and other members of the Parvoviridae family are generally described in Kenneth I. Berns, “Parvoviridae: The Viruses and Their Replication,” Chapter 69 in Fields Virology (3d Ed. 1996), the contents of which are incorporated by reference in their entirety.

AAV have proven to be useful as a biological tool due to their relatively simple structure, their ability to infect a wide range of cells (including quiescent and dividing cells) without integration into the host genome and without replicating, and their relatively benign immunogenic profile. The genome of the virus may be manipulated to contain a minimum of components for the assembly of a functional recombinant virus, or viral particle, which is loaded with or engineered to target a particular tissue and express or deliver a desired payload. The genome of the virus may be modified to contain a minimum of components for the assembly of a functional recombinant virus, or viral particle, which is loaded with or engineered to express or deliver a desired nucleic acid construct or payload, e.g., a polynucleotide encoding a siRNA, e.g., a SOD1 targeting siRNA which may be delivered to a target cell, tissue, or organism. In some embodiments, the target cell is a CNS cell. In some embodiments, the target tissue is a CNS tissue. The target CNS tissue may be brain tissue. In some embodiments, region of the CNS is a region of the brain or spinal cord, e.g., the parenchyma, the cortex, substantia nigra, caudate cerebellum, striatum, corpus callosum, cerebellum, brain stem caudate-putamen, thalamus, superior colliculus, the spinal cord, or a combination thereof.

Gene therapy presents an alternative approach for ALS, and diseases sharing single-gene etiology, such as Gaucher disease and Dementia with Lewy Bodies and related disorders. AAVs are commonly used in gene therapy approaches as a result of a number of advantageous features. Without wishing to be bound by theory, it is believed in some embodiments, that expression vectors, e.g., an adeno-associated viral vector (AAVs) or AAV particle, e.g., an AAV particle described herein, can be used to administer and/or deliver a siRNA (e.g., a SOD1 targeting siRNA), in order to achieve sustained, high concentrations, allowing for longer lasting efficacy, fewer dose treatments, broad biodistribution, and/or more consistent levels of the siRNA, relative to a non-AAV therapy.

As demonstrated in some of the Examples herein below, certain AAV capsid variants described herein show multiple advantages over wild-type AAV9, including (i) increased penetrance through the blood brain barrier following intravenous administration, (ii) wider distribution throughout the multiple brain regions, e.g., frontal cortex, sensory cortex, motor cortex, putamen, thalamus, cerebellar cortex, dentate nucleus, caudate, and/or hippocampus, and/or (iii) elevated payload expression in multiple brain regions. Without wishing to be being bound by theory, it is believed that these advantages may be due, in part, to the dissemination of the AAV capsid variants through the brain vasculature. In some embodiments, the AAV capsids described herein enhance the delivery of a payload, e.g., a polynucleotide encoding a siRNA, e.g., a SOD1 targeting siRNA as described herein, to multiple regions of the brain including for example, the frontal cortex, sensory cortex, motor cortex, putamen, thalamus, cerebellar cortex, dentate nucleus, caudate, and/or hippocampus. In some embodiments, enhance the expression of a payload, e.g., a SOD1 targeting siRNA described herein, to multiple cell types in the CNS, e.g., neurons, oligodendrocytes, and/or glial cells. Without wishing to be bound by theory, an AAV particle comprising an AAV capsid polypeptide, e.g., an AAV capsid variant described herein, for the vectorized delivery of a SOD1 targeting siRNA described here can result in increased penetrance through the blood brain barrier, e.g., following intravenous administration, and/or increased biodistribution of the SOD1 targeting siRNA in the central nervous system, e.g., the brain and the spinal cord.

### II. Compositions

### Vectors

In some embodiments, the siRNA molecules described herein can be inserted into, or encoded by, vectors such as plasmids or viral vectors. Preferably, the siRNA molecules are inserted into, or encoded by, viral vectors.

Viral vectors may be Herpesvirus (HSV) vectors, retroviral vectors, adenoviral vectors, adeno-associated viral vectors, lentiviral vectors, and the like. In some specific embodiments, the viral vectors are AAV vectors.

Adeno-associated viral (AAV) Particles

AAV has a genome of about 5,000 nucleotides in length which contains two open reading frames encoding the proteins responsible for replication (Rep) and the structural protein of the capsid (Cap). The open reading frames are flanked by two Inverted Terminal Repeat (ITR) sequences, which serve as the origin of replication of the viral genome. The wild-type AAV viral genome comprises nucleotide sequences for two open reading frames, one for the four non-structural Rep proteins (Rep78, Rep68, Rep52, Rep40, encoded by Rep genes) and one for the three capsid, or structural, proteins (VP1, VP2, VP3, encoded by capsid genes or Cap genes). The Rep proteins are important for replication and packaging, while the capsid proteins are assembled to create the protein shell of the AAV, or AAV capsid. Alternative splicing and alternate initiation codons and promoters result in the generation of four different Rep proteins from a single open reading frame and the generation of three capsid proteins from a single open reading frame. Though it varies by AAV serotype, as a non-limiting example, for AAV9/hu.14 (SEQ ID NO: 123 of U.S. Pat. No. 7,906,111, the contents of which are herein incorporated by reference in their entirety; SEQ ID NO: 138, Table 2, herein) VP1 refers to amino acids 1-736, VP2 refers to amino acids 138-736, and VP3 refers to amino acids 203-736. As another non-limiting example, VP1 refers to amino acids 1-743 numbered according to SEQ ID NO: 1, VP2 refers to amino acids 138-743 numbered according to SEQ ID NO: 1, and VP3 refers to amino acids 203-743 numbered according to SEQ ID NO: 1. As another non-limiting example, VP1 refers to amino acids 1-742 numbered according to SEQ ID NO: 981 or 982, VP2 refers to amino acids 138-742 numbered according to SEQ ID NO: 981 or 982, and VP3 refers to amino acids 203-742 numbered according to SEQ ID NO: 981 or 982. In other words, VP1 is the full-length capsid sequence, while VP2 and VP3 are shorter components of the whole. As a result, changes in the sequence in the VP3 region, are also changes to VP1 and VP2, however, the percent difference as compared to the parent sequence will be greatest for VP3 since it is the shortest sequence of the three. Though described here in relation to the amino acid sequence, the nucleic acid sequence encoding these proteins can be similarly described. Together, the three capsid proteins assemble to create the AAV capsid protein. While not wishing to be bound by theory, the AAV capsid protein typically comprises a molar ratio of 1:1:10 of VP1:VP2:VP3. As used herein, an “AAV serotype” is defined primarily by the AAV capsid. In some instances, the ITRs are also specifically described by the AAV serotype (e.g., AAV2/9).

The AAV vector typically requires a co-helper (e.g., adenovirus) to undergo productive infection in cells. In the absence of such helper functions, the AAV virions essentially enter host cells but do not integrate into the cells' genome.

AAV vectors have been investigated for delivery of gene therapeutics because of several unique features. Non-limiting examples of the features include (i) the ability to infect both dividing and non-dividing cells; (ii) a broad host range for infectivity, including human cells; (iii) wild-type AAV has not been associated with any disease and has not been shown to replicate in infected cells; (iv) the lack of cell-mediated immune response against the vector, and (v) the non-integrative nature in a host chromosome thereby reducing potential for long-term genetic alterations. Moreover, infection with AAV vectors has minimal influence on changing the pattern of cellular gene expression (Stilwell and Samulski et al., Biotechniques, 2003, 34, 148, the contents of which are herein incorporated by reference in their entirety).

Typically, AAV particle for delivery of an siRNA, e.g., an siRNA for targeting SOD1 as described herein, may be recombinant viral vectors which are replication defective as they lack sequences encoding functional Rep and Cap proteins within the viral genome. In some cases, the defective AAV genome may lack most or all coding sequences and essentially only contain one or two AAV ITR sequences and a payload sequence. In certain embodiments, the viral genome encodes a siRNA for targeting SOD1.

In some embodiments, the AAV particles of the present disclosure may be introduced into mammalian cells.

AAV particles may be modified to enhance the efficiency of delivery. Such modified AAV particles of the present disclosure can be packaged efficiently and can be used to successfully infect the target cells at high frequency and with minimal toxicity.

In other embodiments, AAV particles of the present disclosure may be used to deliver a siRNA (e.g., a siRNA targeting SOD1 as described herein) to the central nervous system (see, e.g., U.S. Pat. No. 6,180,613; the contents of which are herein incorporated by reference in their entirety) or to specific tissues of the CNS.

It is understood that the compositions described herein may have additional conservative or non-essential amino acid substitutions, which do not have a substantial effect on their functions.

AAV Capsids and Variants thereof.

In some embodiments, an AAV particle, e.g., an AAV particle for the vectorized delivery of an siRNA described herein (e.g., an siRNA for targeting SOD1), may comprise an AAV capsid polypeptide, e.g., an AAV capsid variant.

In some embodiments, the AAV capsid polypeptide, e.g., AAV capsid variant, allows for blood brain barrier penetration following intravenous administration. In some embodiments, the AAV capsid, e.g., AAV capsid variant, allows for blood brain barrier penetration following focused ultrasound (FUS), e.g., coupled with the intravenous administration of microbubbles (FUS-MB), or MRI-guided FUS coupled with intravenous administration. In some embodiments the AAV capsid, e.g., AAV capsid variant allows for increased distribution to a brain region. In some embodiments, the brain region comprises a frontal cortex, sensory cortex, motor cortex, caudate, dentate nucleus, cerebellar cortex, cerebral cortex, brain stem, hippocampus, thalamus, putamen, or a combination thereof. In some embodiments, the AAV capsid, e.g., AAV capsid variant allows for preferential transduction in a brain region relative to the transduction in the dorsal root ganglia (DRG).

In some embodiments the AAV capsid polypeptide, e.g., AAV capsid variant allows for increased distribution to a spinal cord region. In some embodiments, the spinal region comprises a cervical spinal cord region, thoracic spinal cord region, and/or lumbar spinal cord region.

In some embodiments, the AAV capsid polypeptide, e.g., an AAV capsid variant comprises a VOY101 capsid polypeptide, a PHPeB capsid polypeptide, an AAVPHP.B (PHP.B) capsid polypeptide, a AAVPHP.N (PHP.N) capsid polypeptide, an AAV1 capsid polypeptide, an AAV2 capsid polypeptide, an AAV5 capsid polypeptide, an AAV9 capsid polypeptide, an AAV9 K449R capsid polypeptide, an AAVrh10 capsid polypeptide, or a functional variant thereof. In some embodiments, the AAV capsid polypeptide, e.g., AAV capsid variant, comprises an amino acid sequence of any of the AAV capsid polypeptides in Table 1, or an amino acid sequence substantially identical (e.g., having at least 70%, 75%, 80%, 85%, 90%, 92%, 95%, 97%, 98%, or 99% sequence identity) thereto. In some embodiments, the nucleotide sequence encoding the AAV capsid polypeptide comprises any one of the nucleotide sequences in Table 1, or a nucleotide sequence substantially identical (e.g., having at least 70%, 75%, 80%, 85%, 90%, 92%, 95%, 97%, 98%, or 99% sequence identity) thereto.

TABLE 1
Exemplary full length capsid sequences

| | SEQ | |
|---|---|---|
| | ID | |
| Description | NO: | Sequence Information |
| VOY101 | 1 | MAADGYLPDWLEDNLSEGIREWWALKPGAPQPKANQQHQDNARGLVLPGYKYLGPGNGL |
| | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSDGTL |
| | | AVPFKAQAQTGWVQNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKENSKRWNPEIQY |
| | | TSNYYKSNNVEFAVNTEGVYSEPRPIGTRYLTRNL |
| AAV9/hu.14 | 11 | MAADGYLPDWLEDNLSEGIREWWALKPGAPQPKANQQHQDNARGLVLPGYKYLGPGNGL |
| K449R | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRINFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSRTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSAQAQ |
| | | AQTGWVQNQGILPGMVWQDRDVYLQGPIWAKIPHTDGNFHPSPLMGGFGMKHPPPQILI |
| | | KNTPVPADPPTAFNKDKLNSFITQYSTGQVSVEIEWELQKENSKRWNPEIQYTSNYYKS |
| | | NNVEFAVNTEGVYSEPRPIGTRYLTRNL |
| AAV9/hu.14 | 138 | MAADGYLPDWLEDNLSEGIREWWALKPGAPQPKANQQHQDNARGLVLPGYKYLGPGNGL |
| WT | | DKGEPVNAADAAALEHDKAYDQQLKAGDNPYLKYNHADAEFQERLKEDTSFGGNLGRAV |
| (amino acid) | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSAQAQ |
| | | AQTGWVQNQGILPGMVWQDRDVYLQGPIWAKIPHTDGNFHPSPLMGGFGMKHPPPQILI |
| | | KNTPVPADPPTAFNKDKLNSFITQYSTGQVSVEIEWELQKENSKRWNPEIQYTSNYYKS |
| | | NNVEFAVNTEGVYSEPRPIGTRYLTRNL |
| AAV9/hu.14 | 137 | ATGGCTGCCGATGGTTATCTTCCAGATTGGCTCGAGGACAACCTTAGTGAAGGAATTCG |
| WT | | CGAGTGGTGGGCTTTGAAACCTGGAGCCCCTCAACCCAAGGCAAATCAACAACATCAAG |
| (DNA) | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCITAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGACCAATACTTGTACTATCTCTCAAAGACTATTAACG |
| | | GTTCTGGACAGAATCAACAAACGCTAAAATTCAGTGTGGCCGGACCCAGCAACATGGCT |
| | | GTCCAGGGAAGAAACTACATACCTGGACCCAGCTACCGACAACAACGTGTCTCAACCAC |
| | | TGTGACTCAAAACAACAACAGCGAATTTGCTTGGCCTGGAGCTTCTTCTTGGGCTCTCA |
| | | ATGGACGTAATAGCTTGATGAATCCTGGACCTGCTATGGCCAGCCACAAAGAAGGAGAG |
| | | GACCGTTTCTTTCCTTTGTCTGGATCTTTAATTTTTGGCAAACAAGGAACTGGAAGAGA |
| | | CAACGTGGATGCGGACAAAGTCATGATAACCAACGAAGAAGAAATTAAAACTACTAACC |
| | | CGGTAGCAACGGAGTCCTATGGACAAGTGGCCACAAACCACCAGAGTGCCCAAGCACAG |
| | | GCGCAGACCGGCTGGGTTCAAAACCAAGGAATACTTCCGGGTATGGTTTGGCAGGACAG |
| | | AGATGTGTACCTGCAAGGACCCATTTGGGCCAAAATTCCTCACACGGACGGCAACTTTC |
| | | ACCCTTCTCCGCTGATGGGAGGGTTTGGAATGAAGCACCCGCCTCCTCAGATCCTCATC |
| | | AAAAACACACCTGTACCTGCGGATCCTCCAACGGCCTTCAACAAGGACAAGCTGAACTC |
| | | TTTCATCACCCAGTATTCTACTGGCCAAGTCAGCGTGGAGATCGAGTGGGAGCTGCAGA |
| | | AGGAAAACAGCAAGCGCTGGAACCCGGAGATCCAGTACACTTCCAACTATTACAAGTCT |
| | | AATAATGTTGAATTTGCTGTTAATACTGAAGGTGTATATAGTGAACCCCGCCCCATTGG |
| | | CACCAGATACCTGACTCGTAATCTGTAA |
| PHP.eB | 10 | MAADGYLPDWLEDNLSEGIREWWALKPGAPQPKANQQHQDNARGLVLPGYKYLGPGNGL |
| (amino acid) | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSRTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSDGTL |
| | | AVPFKAQAQTGWVQNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKENSKRWNPEIQY |
| | | TSNYYKSNNVEFAVNTEGVYSEPRPIGTRYLTRNL |
| PHP.B | 12 | MAADGYLPDWLEDNLSEGIREWWALKPGAPQPKANQQHQDNARGLVLPGYKYLGPGNGL |
| (amino acid) | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSRTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSAQTL |
| | | AVPFKAQAQTGWVQNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKENSKRWNPEIQY |
| | | TSNYYKSNNVEFAVNTEGVYSEPRPIGTRYLTRNL |
| PHP.N | 13 | MAADGYLPDWLEDNLSEGIREWWALKPGAPQPKANQQHQDNARGLVLPGYKYLGPGNGL |
| (amino acid) | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRINFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSRTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSAQTL |
| | | AVPFSNPAQTGWVQNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKENSKRWNPEIQY |
| | | TSNYYKSNNVEFAVNTEGVYSEPRPIGTRYLTRNL |
| AAVrh10 | 14 | MAADGYLPDWLEDNLSEGIREWWDLKPGAPKPKANQQKQDDGRGLVLPGYKYLGPENGL |
| (amino acid) | | DKGEPVNAADAAALEHDKAYDQQLKAGDNPYLRYNHADAEFQERLQEDTSFGGNLGRAV |
| | | FQAKKRVLEPLGLVEEGAKTAPGKKRPVEPSPQRSPDSSTGIGKKGQQPAKKRLNFGQT |
| | | GDSESVPDPQPIGEPPAGPSGLGSGTMAAGGGAPMADNNEGADGVGSSSGNWHCDSTWL |
| | | GDRVITTSTRTWALPTYNNHLYKQISNGTSGGSTNDNTYFGYSTPWGYFDFNRFHCHFS |
| | | PRDWQRLINNNWGFRPKRLNFKLFNIQVKEVTQNEGTKTIANNLTSTIQVFTDSEYQLP |
| | | YVLGSAHQGCLPPFPADVFMIPQYGYLTLNNGSQAVGRSSFYCLEYFPSQMLRTGNNFE |
| | | FSYQFEDVPFHSSYAHSQSLDRLMNPLIDQYLYYLSRTQSTGGTAGTQQLLFSQAGPNN |
| | | MSAQAKNWLPGPCYRQQRVSTTLSQNNNSNFAWTGATKYHLNGRDSLVNPGVAMATHKD |
| | | DEERFFPSSGVLMFGKQGAGKDNVDYSSVMLTSEEEIKTTNPVATEQYGVVADNLQQQN |
| | | AAPIVGAVNSQGALPGMVWQNRDVYLQGPIWAKIPHTDGNFHPSPLMGGFGLKHPPPQI |
| | | LIKNTPVPADPPTTFSQAKLASFITQYSTGQVSVEIEWELQKENSKRWNPEIQYTSNYY |
| | | KSTNVDFAVNTDGTYSEPRPIGTRYLTRNL |
| AAVrh10 | 15 | ATGGCTGCCGATGGTTATCTTCCAGATTGGCTCGAGGACAACCTCTCTGAGGGCATTCG |
| (DNA) | | CGAGTGGTGGGACTTGAAACCTGGAGCCCCGAAACCCAAAGCCAACCAGCAAAAGCAGG |
| | | ACGACGGCCGGGGTCTGGTGCTTCCTGGCTACAAGTACCTCGGACCCTTCAACGGACTC |
| | | GACAAGGGGGAGCCCGTCAACGCGGCGGACGCAGCGGCCCTCGAGCACGACAAGGCCTA |
| | | CGACCAGCAGCTCAAAGCGGGTGACAATCCGTACCTGCGGTATAACCACGCCGACGCCG |
| | | AGTTTCAGGAGCGTCTGCAAGAAGATACGTCTTTTGGGGGCAACCTCGGGCGAGCAGTC |
| | | TTCCAGGCCAAGAAGCGGGTTCTCGAACCTCTCGGTCTGGTTGAGGAAGGCGCTAAGAC |
| | | GGCTCCTGGAAAGAAGAGACCGGTAGAGCCATCACCCCAGCGTTCTCCAGACTCCTCTA |
| | | CGGGCATCGGCAAGAAAGGCCAGCAGCCCGCGAAAAAGAGACTCAACTTTGGGCAGACT |
| | | GGCGACTCAGAGTCAGTGCCCGACCCTCAACCAATCGGAGAACCCCCCGCAGGCCCCTC |
| | | TGGTCTGGGATCTGGTACAATGGCTGCAGGCGGTGGCGCTCCAATGGCAGACAATAACG |
| | | AAGGCGCCGACGGAGTGGGTAGTTCCTCAGGAAATTGGCATTGCGATTCCACATGGCTG |
| | | GGCGACAGAGTCATCACCACCAGCACCCGAACCTGGGCCCTCCCCACCTACAACAACCA |
| | | CCTCTACAAGCAAATCTCCAACGGGACTTCGGGAGGAAGCACCAACGACAACACCTACT |
| | | TCGGCTACAGCACCCCCTGGGGGTATTTTGACTTTAACAGATTCCACTGCCACTTCTCA |
| | | CCACGTGACTGGCAGCGACTCATCAACAACAACTGGGGATTCCGGCCCAAGAGACTCAA |
| | | CTTCAAGCTCTTCAACATCCAGGTCAAGGAGGTCACGCAGAATGAAGGCACCAAGACCA |
| | | TCGCCAATAACCTTACCAGCACGATTCAGGTCTTTACGGACTCGGAATACCAGCTCCCG |
| | | TACGTCCTCGGCTCTGCGCACCAGGGCTGCCTGCCTCCGTTCCCGGCGGACGTCTTCAT |
| | | GATTCCTCAGTACGGGTACCTGACTCTGAACAATGGCAGTCAGGCCGTGGGCCGTTCCT |
| | | CCTTCTACTGCCTGGAGTACTTTCCTTCTCAAATGCTGAGAACGGGCAACAACTTTGAG |
| | | TTCAGCTACCAGTTTGAGGACGTGCCTTTTCACAGCAGCTACGCGCACAGCCAAAGCCT |
| | | GGACCGGCTGATGAACCCCCTCATCGACCAGTACCTGTACTACCTGTCTCGGACTCAGT |
| | | CCACGGGAGGTACCGCAGGAACTCAGCAGTTGCTATTTTCTCAGGCCGGGCCTAATAAC |
| | | ATGTCGGCTCAGGCCAAAAACTGGCTACCCGGGCCCTGCTACCGGCAGCAACGCGTCTC |
| | | CACGACACTGTCGCAAAATAACAACAGCAACTTTGCCTGGACCGGTGCCACCAAGTATC |
| | | ATCTGAATGGCAGAGACTCTCTGGTAAATCCCGGTGTCGCTATGGCAACCCACAAGGAC |
| | | GACGAAGAGCGATTTTTTCCGTCCAGCGGAGTCTTAATGTTTGGGAAACAGGGAGCTGG |
| | | AAAAGACAACGTGGACTATAGCAGCGTTATGCTAACCAGTGAGGAAGAAATTAAAACCA |
| | | CCAACCCAGTGGCCACAGAACAGTACGGCGTGGTGGCCGATAACCTGCAACAGCAAAAC |
| | | GCCGCTCCTATTGTAGGGGCCGTCAACAGTCAAGGAGCCTTACCTGGCATGGTCTGGCA |
| | | GAACCGGGACGTGTACCTGCAGGGTCCTATCTGGGCCAAGATTCCTCACACGGACGGAA |
| | | ACTTTCATCCCTCGCCGCTGATGGGAGGCTTTGGACTGAAACACCCGCCTCCTCAGATC |
| | | CTGATTAAGAATACACCTGTTCCCGCGGATCCTCCAACTACCTTCAGTCAAGCTAAGCT |
| | | GGCGTCGTTCATCACGCAGTACAGCACCGGACAGGTCAGCGTGGAAATTGAATGGGAGC |
| | | TGCAGAAAGAAAACAGCAAACGCTGGAACCCAGAGATTCAATACACTTCCAACTACTAC |
| | | AAATCTACAAATGTGGACTTTGCTGTTAACACAGATGGCACTTATTCTGAGCCTCGCCC |
| | | CATCGGCACCCGTTACCTCACCCGTAATCTGTAA |
| AAV2 | 16 | MAADGYLPDWLEDTLSEGIRQWWKLKPGPPPPKPAERHKDDSRGLVLPGYKYLGPENGL |
| (amino acid) | | DKGEPVNEADAAALEHDKAYDRQLDSGDNPYLKYNHADAEFQERLKEDTSFGGNLGRAV |
| | | FQAKKRVLEPLGLVEEPVKTAPGKKRPVEHSPVEPDSSSGTGKAGQQPARKRLNFGQTG |
| | | DADSVPDPQPLGQPPAAPSGLGTNTMATGSGAPMADNNEGADGVGNSSGNWHCDSTWMG |
| | | DRVITTSTRTWALPTYNNHLYKQISSQSGASNDNHYFGYSTPWGYFDFNRFHCHFSPRD |
| | | WQRLINNNWGFRPKRLNFKLFNIQVKEVTQNDGITTIANNLTSTVQVFTDSEYQLPYVL |
| | | GSAHQGCLPPFPADVFMVPQYGYLTLNNGSQAVGRSSFYCLEYFPSQMLRTGNNFTESY |
| | | TFEDVPFHSSYAHSQSLDRLMNPLIDQYLYYLSRTNTPSGTTTQSRLQFSQAGASDIRD |
| | | QSRNWLPGPCYRQQRVSKTSADNNNSEYSWTGATKYHLNGRDSLVNPGPAMASHKDDEE |
| | | KFFPQSGVLIFGKQGSEKTNVDIEKVMITDEEEIRTTNPVATEQYGSVSTNLQRGNRQA |
| | | ATADVNTQGVLPGMVWQDRDVYLQGPIWAKIPHTDGHFHPSPLMGGFGLKHPPPQILIK |
| | | NTPVPANPSTTFSAAKFASFITQYSTGQVSVEIEWELQKENSKRWNPEIQYTSNYNKSV |
| | | NVDFTVDINGVYSEPRPIGTRYLTRNL |
| AAV2 | 17 | ATGGCTGCCGATGGTTATCTTCCAGATTGGCTCGAGGACACTCTCTCTGAAGGAATAAG |
| (DNA) | | ACAGTGGTGGAAGCTCAAACCTGGCCCACCACCACCAAAGCCCGCAGAGCGGCATAAGG |
| | | ACGACAGCAGGGGTCTTGTGCTTCCTGGGTACAAGTACCTCGGACCCTTCAACGGACTC |
| | | GACAAGGGAGAGCCGGTCAACGAGGCAGACGCCGCGGCCCTCGAGCACGACAAAGCCTA |
| | | CGACCGGCAGCTCGACAGCGGAGACAACCCGTACCTCAAGTACAACCACGCCGACGCGG |
| | | AGTTTCAGGAGCGCCTTAAAGAAGATACGTCTTTTGGGGGCAACCTCGGACGAGCAGTC |
| | | TTCCAGGCGAAAAAGAGGGTTCTTGAACCTCTGGGCCTGGTTGAGGAACCTGTTAAGAC |
| | | GGCTCCGGGAAAAAAGAGGCCGGTAGAGCACTCTCCTGTGGAGCCAGACTCCTCCTCGG |
| | | GAACCGGAAAGGCGGGCCAGCAGCCTGCAAGAAAAAGATTGAATTTTGGTCAGACTGGA |
| | | GACGCAGACTCAGTACCTGACCCCCAGCCTCTCGGACAGCCACCAGCAGCCCCCTCTGG |
| | | TCTGGGAACTAATACGATGGCTACAGGCAGTGGCGCACCAATGGCAGACAATAACGAGG |
| | | GCGCCGACGGAGTGGGTAATTCCTCGGGAAATTGGCATTGCGATTCCACATGGATGGGC |
| | | GACAGAGTCATCACCACCAGCACCCGAACCTGGGCCCTGCCCACCTACAACAACCACCT |
| | | CTACAAACAAATTTCCAGCCAATCAGGAGCCTCGAACGACAATCACTACTTTGGCTACA |
| | | GCACCCCTTGGGGGTATTTTGACTTCAACAGATTCCACTGCCACTTTTCACCACGTGAC |
| | | TGGCAAAGACTCATCAACAACAACTGGGGATTCCGACCCAAGAGACTCAACTTCAAGCT |
| | | CTTTAACATTCAAGTCAAAGAGGTCACGCAGAATGACGGTACGACGACGATTGCCAATA |
| | | ACCTTACCAGCACGGTTCAGGTGTTTACTGACTCGGAGTACCAGCTCCCGTACGTCCTC |
| | | GGCTCGGCGCATCAAGGATGCCTCCCGCCGTTCCCAGCAGACGTCTTCATGGTGCCACA |
| | | GTATGGATACCTCACCCTGAACAACGGGAGTCAGGCAGTAGGACGCTCTTCATTTTACT |
| | | GCCTGGAGTACTTTCCTTCTCAGATGCTGCGTACCGGAAACAACTTTACCTTCAGCTAC |
| | | ACTTTTGAGGACGTTCCTTTCCACAGCAGCTACGCTCACAGCCAGAGTCTGGACCGTCT |
| | | CATGAATCCTCTCATCGACCAGTACCTGTATTACTTGAGCAGAACAAACACTCCAAGTG |
| | | GAACCACCACGCAGTCAAGGCTTCAGTTTTCTCAGGCCGGAGCGAGTGACATTCGGGAC |
| | | CAGTCTAGGAACTGGCTTCCTGGACCCTGTTACCGCCAGCAGCGAGTATCAAAGACATC |
| | | TGCGGATAACAACAACAGTGAATACTCGTGGACTGGAGCTACCAAGTACCACCTCAATG |
| | | GCAGAGACTCTCTGGTGAATCCGGGCCCGGCCATGGCAAGCCACAAGGACGATGAAGAA |
| | | AAGTTTTTTCCTCAGAGCGGGGTTCTCATCTTTGGGAAGCAAGGCTCAGAGAAAACAAA |
| | | TGTGGACATTGAAAAGGTCATGATTACAGACGAAGAGGAAATCAGGACAACCAATCCCG |
| | | TGGCTACGGAGCAGTATGGTTCTGTATCTACCAACCTCCAGAGAGGCAACAGACAAGCA |
| | | GCTACCGCAGATGTCAACACACAAGGCGTTCTTCCAGGCATGGTCTGGCAGGACAGAGA |
| | | TGTGTACCTTCAGGGGCCCATCTGGGCAAAGATTCCACACACGGACGGACATTTTCACC |
| | | CCTCTCCCCTCATGGGTGGATTCGGACTTAAACACCCTCCTCCACAGATTCTCATCAAG |
| | | AACACCCCGGTACCTGCGAATCCTTCGACCACCTTCAGTGCGGCAAAGTTTGCTTCCTT |
| | | CATCACACAGTACTCCACGGGACAGGTCAGCGTGGAGATCGAGTGGGAGCTGCAGAAGG |
| | | AAAACAGCAAACGCTGGAATCCCGAAATTCAGTACACTTCCAACTACAACAAGTCTGTT |
| | | AATGTGGACTTTACTGTGGACACTAATGGCGTGTATTCAGAGCCTCGCCCCATTGGCAC |
| | | CAGATACCTGACTCGTAATCTGTAA |
| AAV1 | 18 | MAADGYLPDWLEDNLSEGIREWWDLKPGAPKPKANQQKQDDGRGLVLPGYKYLGPFNGL |
| (amino acid) | | DKGEPVNAADAAALEHDKAYDQQLKAGDNPYLRYNHADAEFQERLQEDTSFGGNLGRAV |
| | | FQAKKRVLEPLGLVEEGAKTAPGKKRPVEQSPQEPDSSSGIGKTGQQPAKKRINFGQTG |
| | | DSESVPDPQPLGEPPATPAAVGPTTMASGGGAPMADNNEGADGVGNASGNWHCDSTWLG |
| | | DRVITTSTRTWALPTYNNHLYKQISSASTGASNDNHYFGYSTPWGYFDFNRFHCHFSPR |
| | | DWQRLINNNWGFRPKRLNFKLFNIQVKEVTTNDGVTTIANNLTSTVQVFSDSEYQLPYV |
| | | LGSAHQGCLPPFPADVFMIPQYGYLTLNNGSQAVGRSSFYCLEYFPSQMLRTGNNFTFS |
| | | YTFEEVPFHSSYAHSQSLDRLMNPLIDQYLYYLNRTQNQSGSAQNKDLLFSRGSPAGMS |
| | | VQPKNWLPGPCYRQQRVSKTKTDNNNSNFTWTGASKYNLNGRESIINPGTAMASHKDDE |
| | | DKFFPMSGVMIFGKESAGASNTALDNVMITDEEEIKATNPVATERFGTVAVNFQSSSTD |
| | | PATGDVHAMGALPGMVWQDRDVYLQGPIWAKIPHTDGHFHPSPLMGGFGLKNPPPQILI |
| | | KNTPVPANPPAEFSATKFASFITQYSTGQVSVEIEWELQKENSKRWNPEVQYTSNYAKS |
| | | ANVDFTVDNNGLYTEPRPIGTRYLTRPL |
| AAV1 | 19 | ATGGCTGCCGATGGTTATCTTCCAGATTGGCTCGAGGACAACCTCTCTGAGGGCATTCG |
| (DNA) | | CGAGTGGTGGGACTTGAAACCTGGAGCCCCGAAGCCCAAAGCCAACCAGCAAAAGCAGG |
| | | ACGACGGCCGGGGTCTGGTGCTTCCTGGCTACAAGTACCTCGGACCCTTCAACGGACTC |
| | | GACAAGGGGGAGCCCGTCAACGCGGCGGACGCAGCGGCCCTCGAGCACGACAAGGCCTA |
| | | CGACCAGCAGCTCAAAGCGGGTGACAATCCGTACCTGCGGTATAACCACGCCGACGCCG |
| | | AGTTTCAGGAGCGTCTGCAAGAAGATACGTCTTTTGGGGGCAACCTCGGGCGAGCAGTC |
| | | TTCCAGGCCAAGAAGCGGGTTCTCGAACCTCTCGGTCTGGTTGAGGAAGGCGCTAAGAC |
| | | GGCTCCTGGAAAGAAACGTCCGGTAGAGCAGTCGCCACAAGAGCCAGACTCCTCCTCGG |
| | | GCATCGGCAAGACAGGCCAGCAGCCCGCTAAAAAGAGACTCAATTTTGGTCAGACTGGC |
| | | GACTCAGAGTCAGTCCCCGATCCACAACCTCTCGGAGAACCTCCAGCAACCCCCGCTGC |
| | | TGTGGGACCTACTACAATGGCTTCAGGCGGTGGCGCACCAATGGCAGACAATAACGAAG |
| | | GCGCCGACGGAGTGGGTAATGCCTCAGGAAATTGGCATTGCGATTCCACATGGCTGGGC |
| | | GACAGAGTCATCACCACCAGCACCCGCACCTGGGCCTTGCCCACCTACAATAACCACCT |
| | | CTACAAGCAAATCTCCAGTGCTTCAACGGGGGCCAGCAACGACAACCACTACTTCGGCT |
| | | ACAGCACCCCCTGGGGGTATTTTGATTTCAACAGATTCCACTGCCACTTTTCACCACGT |
| | | GACTGGCAGCGACTCATCAACAACAATTGGGGATTCCGGCCCAAGAGACTCAACTTCAA |
| | | ACTCTTCAACATCCAAGTCAAGGAGGTCACGACGAATGATGGCGTCACAACCATCGCTA |
| | | ATAACCTTACCAGCACGGTTCAAGTCTTCTCGGACTCGGAGTACCAGCTTCCGTACGTC |
| | | CTCGGCTCTGCGCACCAGGGCTGCCTCCCTCCGTTCCCGGCGGACGTGTTCATGATTCC |
| | | GCAATACGGCTACCTGACGCTCAACAATGGCAGCCAAGCCGTGGGACGTTCATCCTTTT |
| | | ACTGCCTGGAATATTTCCCTTCTCAGATGCTGAGAACGGGCAACAACTTTACCTTCAGC |
| | | TACACCTTTGAGGAAGTGCCTTTCCACAGCAGCTACGCGCACAGCCAGAGCCTGGACCG |
| | | GCTGATGAATCCTCTCATCGACCAATACCTGTATTACCTGAACAGAACTCAAAATCAGT |
| | | CCGGAAGTGCCCAAAACAAGGACTTGCTGTTTAGCCGTGGGTCTCCAGCIGGCATGTCT |
| | | GTTCAGCCCAAAAACTGGCTACCTGGACCCTGTTATCGGCAGCAGCGCGTTTCTAAAAC |
| | | AAAAACAGACAACAACAACAGCAATTTTACCTGGACTGGTGCTTCAAAATATAACCTCA |
| | | ATGGGCGTGAATCCATCATCAACCCTGGCACTGCTATGGCCTCACACAAAGACGACGAA |
| | | GACAAGTTCTTTCCCATGAGCGGTGTCATGATTTTTGGAAAAGAGAGCGCCGGAGCTTC |
| | | AAACACTGCATTGGACAATGTCATGATTACAGACGAAGAGGAAATTAAAGCCACTAACC |
| | | CTGTGGCCACCGAAAGATTTGGGACCGTGGCAGTCAATTTCCAGAGCAGCAGCACAGAC |
| | | CCTGCGACCGGAGATGTGCATGCTATGGGAGCATTACCTGGCATGGTGTGGCAAGATAG |
| | | AGACGTGTACCTGCAGGGTCCCATTTGGGCCAAAATTCCTCACACAGATGGACACTTTC |
| | | ACCCGTCTCCTCTTATGGGCGGCTTTGGACTCAAGAACCCGCCTCCTCAGATCCTCATC |
| | | AAAAACACGCCTGTTCCTGCGAATCCTCCGGCGGAGTTTTCAGCTACAAAGTTTGCTTC |
| | | ATTCATCACCCAATACTCCACAGGACAAGTGAGTGTGGAAATTGAATGGGAGCTGCAGA |
| | | AAGAAAACAGCAAGCGCTGGAATCCCGAAGTGCAGTACACATCCAATTATGCAAAATCT |
| | | GCCAACGTTGATTTTACTGTGGACAACAATGGACTTTATACTGAGCCTCGCCCCATTGG |
| | | CACCCGTTACCTTACCCGTCCCCTGTAA |
| AAV5 | 20 | MSFVDHPPDWLEEVGEGLREFLGLEAGPPKPKPNQQHQDQARGLVLPGYNYLGPGNGLD |
| (amino acid) | | RGEPVNRADEVAREHDISYNEQLEAGDNPYLKYNHADAEFQEKLADDTSFGGNLGKAVF |
| | | QAKKRVLEPFGLVEEGAKTAPTGKRIDDHFPKRKKARTEEDSKPSTSSDAEAGPSGSQQ |
| | | LQIPAQPASSLGADTMSAGGGGPLGDNNQGADGVGNASGDWHCDSTWMGDRVVTKSTRT |
| | | WVLPSYNNHQYREIKSGSVDGSNANAYFGYSTPWGYFDFNRFHSHWSPRDWQRLINNYW |
| | | GFRPRSLRVKIFNIQVKEVTVQDSTTTIANNLTSTVQVFTDDDYQLPYVVGNGTEGCLP |
| | | AFPPQVFTLPQYGYATLNRDNTENPTERSSFFCLEYFPSKMLRTGNNFEFTYNFEEVPF |
| | | HSSFAPSQNLFKLANPLVDQYLYRFVSTNNTGGVQFNKNLAGRYANTYKNWFPGPMGRT |
| | | QGWNLGSGVNRASVSAFATTNRMELEGASYQVPPQPNGMTNNLQGSNTYALENTMIENS |
| | | QPANPGTTATYLEGNMLITSESETQPVNRVAYNVGGQMATNNQSSTTAPATGTYNLQEI |
| | | VPGSVWMERDVYLQGPIWAKIPETGAHFHPSPAMGGFGLKHPPPMMLIKNTPVPGNITS |
| | | FSDVPVSSFITQYSTGQVTVEMEWELKKENSKRWNPEIQYTNNYNDPQFVDFAPDSTGE |
| | | YRTTRPIGTRYLTRPL |
| AAV5 | 21 | ATGTCTTTTGTTGATCACCCTCCAGATTGGTTGGAAGAAGTTGGTGAAGGTCTTCGCGA |
| (DNA) | | GTTTTTGGGCCTTGAAGCGGGCCCACCGAAACCAAAACCCAATCAGCAGCATCAAGATC |
| | | AAGCCCGTGGTCTTGTGCTGCCTGGTTATAACTATCTCGGACCCGGAAACGGTCTCGAT |
| | | CGAGGAGAGCCTGTCAACAGGGCAGACGAGGTCGCGCGAGAGCACGACATCTCGTACAA |
| | | CGAGCAGCTTGAGGCGGGAGACAACCCCTACCTCAAGTACAACCACGCGGACGCCGAGT |
| | | TTCAGGAGAAGCTCGCCGACGACACATCCTTCGGGGGAAACCTCGGAAAGGCAGTCTTT |
| | | CAGGCCAAGAAAAGGGTTCTCGAACCTTTTGGCCTGGTTGAAGAGGGTGCTAAGACGGC |
| | | CCCTACCGGAAAGCGGATAGACGACCACTTTCCAAAAAGAAAGAAGGCCCGGACCGAAG |
| | | AGGACTCCAAGCCTTCCACCTCGTCAGACGCCGAAGCTGGACCCAGCGGATCCCAGCAG |
| | | CTGCAAATCCCAGCCCAACCAGCCTCAAGTTTGGGAGCTGATACAATGTCTGCGGGAGG |
| | | TGGCGGCCCATTGGGCGACAATAACCAAGGTGCCGATGGAGTGGGCAATGCCTCGGGAG |
| | | ATTGGCATTGCGATTCCACGTGGATGGGGGACAGAGTCGTCACCAAGTCCACCCGAACC |
| | | TGGGTGCTGCCCAGCTACAACAACCACCAGTACCGAGAGATCAAAAGCGGCTCCGTCGA |
| | | CGGAAGCAACGCCAACGCCTACTTTGGATACAGCACCCCCTGGGGGTACTTTGACTTTA |
| | | ACCGCTTCCACAGCCACTGGAGCCCCCGAGACTGGCAAAGACTCATCAACAACTACTGG |
| | | GGCTTCAGACCCCGGTCCCTCAGAGTCAAAATCTTCAACATTCAAGTCAAAGAGGTCAC |
| | | GGTGCAGGACTCCACCACCACCATCGCCAACAACCTCACCTCCACCGTCCAAGTGTTTA |
| | | CGGACGACGACTACCAGCTGCCCTACGTCGTCGGCAACGGGACCGAGGGATGCCTGCCG |
| | | GCCTTCCCTCCGCAGGTCTTTACGCTGCCGCAGTACGGTTACGCGACGCTGAACCGCGA |
| | | CAACACAGAAAATCCCACCGAGAGGAGCAGCTTCTTCTGCCTAGAGTACTTTCCCAGCA |
| | | AGATGCTGAGAACGGGCAACAACTTTGAGTTTACCTACAACTTTGAGGAGGTGCCCTTC |
| | | CACTCCAGCTTCGCTCCCAGTCAGAACCTCTTCAAGCTGGCCAACCCGCTGGTGGACCA |
| | | GTACTTGTACCGCTTCGTGAGCACAAATAACACTGGCGGAGTCCAGTTCAACAAGAACC |
| | | TGGCCGGGAGATACGCCAACACCTACAAAAACTGGTTCCCGGGGCCCATGGGCCGAACC |
| | | CAGGGCTGGAACCTGGGCTCCGGGGTCAACCGCGCCAGTGTCAGCGCCTTCGCCACGAC |
| | | CAATAGGATGGAGCTCGAGGGCGCGAGTTACCAGGTGCCCCCGCAGCCGAACGGCATGA |
| | | CCAACAACCTCCAGGGCAGCAACACCTATGCCCTGGAGAACACTATGATCTTCAACAGC |
| | | CAGCCGGCGAACCCGGGCACCACCGCCACGTACCTCGAGGGCAACATGCTCATCACCAG |
| | | CGAGAGCGAGACGCAGCCGGTGAACCGCGTGGCGTACAACGTCGGCGGGCAGATGGCCA |
| | | CCAACAACCAGAGCTCCACCACTGCCCCCGCGACCGGCACGTACAACCTCCAGGAAATC |
| | | GTGCCCGGCAGCGTGTGGATGGAGAGGGACGTGTACCTCCAAGGACCCATCTGGGCCAA |
| | | GATCCCAGAGACGGGGGCGCACTTTCACCCCTCTCCGGCCATGGGCGGATTCGGACTCA |
| | | AACACCCACCGCCCATGATGCTCATCAAGAACACGCCTGTGCCCGGAAATATCACCAGC |
| | | TTCTCGGACGTGCCCGTCAGCAGCTTCATCACCCAGTACAGCACCGGGCAGGTCACCGT |
| | | GGAGATGGAGTGGGAGCTCAAGAAGGAAAACTCCAAGAGGTGGAACCCAGAGATCCAGT |
| | | ACACAAACAACTACAACGACCCCCAGTTTGTGGACTTTGCCCCGGACAGCACCGGGGAA |
| | | TACAGAACCACCAGACCTATCGGAACCCGATACCTTACCCGACCCCTTTAA |
| TTD-001 | 3636 | MAADGYLPDWLEDNLSEGIREWWALKPGAPQPKANQQHQDNARGLVLPGYKYLGPGNGL |
| 9mer peptide | | DKGEPVNAADAAALEHDKAYDQQLKAGDNPYLKYNHADAEFQERLKEDTSFGGNLGRAV |
| underlined, | | FQAKKRLLEPLGLVEEAAKTAPGKKRPVEQSPQEPDSSAGIGKSGAQPAKKRLNFGQTG |
| starts at | | DTESVPDPQPIGEPPAAPSGVGSLTMASGGGAPVADNNEGADGVGSSSGNWHCDSQWLG |
| position 587 | | DRVITTSTRTWALPTYNNHLYKQISNSTSGGSSNDNAYFGYSTPWGYFDFNRFHCHFSP |
| (immediately | | RDWQRLINNNWGFRPKRLNFKLFNIQVKEVTDNNGVKTIANNLTSTVQVFTDSDYQLPY |
| subsequent to | | VLGSAHEGCLPPFPADVFMIPQYGYLTLNDGSQAVGRSSFYCLEYFPSQMLRTGNNFQF |
| position 586); | | SYEFENVPFHSSYAHSQSLDRLMNPLIDQYLYYLSKTINGSGQNQQTLKFSVAGPSNMA |
| 743 aa | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSPLNG |
| | | AVHLYAQAQTGWVQNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKENSKRWNPEIQY |
| | | TSNYYKSNNVEFAVNTEGVYSEPRPIGTRYLTRNL |
| TTD-001 | 3623 | ATGGCTGCCGATGGTTATCTTCCAGATTGGCTCGAGGACAACCTTAGTGAAGGAATTCG |
| 9mer peptide | | CGAGTGGTGGGCTTTGAAACCTGGAGCCCCTCAACCCAAGGCAAATCAACAACATCAAG |
| underlined | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCITAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGACCAATACTTGTACTATCTCTCAAAGACTATTAACG |
| | | GTTCTGGACAGAATCAACAAACGCTAAAATTCAGTGTGGCCGGACCCAGCAACATGGCT |
| | | GTCCAGGGAAGAAACTACATACCTGGACCCAGCTACCGACAACAACGTGTCTCAACCAC |
| | | TGTGACTCAAAACAACAACAGCGAATTTGCTTGGCCTGGAGCTTCTTCTTGGGCTCTCA |
| | | ATGGACGTAATAGCTTGATGAATCCTGGACCTGCTATGGCCAGCCACAAAGAAGGAGAG |
| | | GACCGTTTCTTTCCTTTGTCTGGATCTTTAATTTTTGGCAAACAAGGAACTGGAAGAGA |
| | | CAACGTGGATGCGGACAAAGTCATGATAACCAACGAAGAAGAAATTAAAACTACTAACC |
| | | CGGTAGCAACGGAGTCCTATGGACAAGTGGCCACAAACCACCAGAGTCCGCTTAATGGT |
| | | GCCGTCCATCTTTATGCTCAGGCGCAGACCGGCTGGGTTCAAAACCAAGGAATACTTCC |
| | | GGGTATGGTTTGGCAGGACAGAGATGTGTACCTGCAAGGACCCATTTGGGCCAAAATTC |
| | | CTCACACGGACGGCAACTTTCACCCTTCTCCGCTGATGGGAGGGTTTGGAATGAAGCAC |
| | | CCGCCTCCTCAGATCCTCATCAAAAACACACCTGTACCTGCCGATCCTCCAACGGCCTT |
| | | CAACAAGGACAAGCTGAACTCTTTCATCACCCAGTATTCTACTGGCCAAGTCAGCGTGG |
| | | AGATCGAGTGGGAGCTGCAGAAGGAAAACAGCAAGCGGTGGAACCCGGAGATCCAGTAC |
| | | ACTTCCAACTATTACAAGTCTAATAATGTTGAATTTGCTGTTAATACTGAAGGTGTATA |
| | | TAGTGAACCCCGCCCCATTGGCACCAGATACCTGACTCGTAATCTGTAA |

In some embodiments, the AAV capsid polypeptide, e.g., the AAV capsid variant, comprises the amino acid sequence of any of SEQ ID NOs: 1, 10, 11, 138, 12, 13, 14, 16, 18, 20, 3636, or an amino acid sequence substantially identical (e.g., having at least 70%, 75%, 80%, 85%, 90%, 92%, 95%, 97%, 98%, or 99% sequence identity) thereto. In some embodiments the AAV capsid polypeptide, e.g., the AAV capsid variant, comprises an amino acid sequence comprising at least one, two, or three modifications, e.g., substitutions (e.g., conservative substitutions), insertions, or deletions, but no more than 30, 20, or 10 modifications, e.g., substitutions (e.g., conservative substitutions), insertions, or deletions, relative to the amino acid sequence of any of SEQ ID NOs: 1, 10, 11, 138, 12, 13, 14, 16, 18, 20, or 3636. In some embodiments, the AAV capsid polypeptide, e.g., the AAV capsid variant, comprises an amino acid sequence encoded by the nucleotide sequence of any of SEQ ID NOs: 137, 15, 17, 19, 21, 3623, or a nucleotide sequence substantially identical (e.g., having at least 70%, 75%, 80%, 85%, 90%, 92%, 95%, 97%, 98%, or 99% sequence identity) thereto.

In some embodiments, the nucleotide sequence encoding the AAV capsid polypeptide, e.g., the AAV capsid variant, comprises the nucleotide sequence of any of SEQ ID NOs: 137, 15, 17, 19, 21, or 3623, or a nucleotide sequence substantially identical (e.g., having at least 70%, 75%, 80%, 85%, 90%, 92%, 95%, 97%, 98%, or 99% sequence identity) thereto. In some embodiments, the nucleotide sequence encoding the AAV capsid polypeptide, e.g., the AAV capsid variant, comprises a nucleotide sequence comprising at least one, two, or three modifications, e.g., substitutions, insertions, or deletions, but no more than 30, 20, or 10 modifications, e.g., substitutions, insertions, or deletions, relative to the nucleotide sequence of any of SEQ ID NOs: 137, 15, 17, 19, 21, or 3623.

In some embodiments, the AAV capsid polypeptide, e.g., the AAV capsid variant, comprises the amino acid sequence of SEQ ID NO: 138 or an amino acid sequence substantially identical (e.g., having at least 70%, 75%, 80%, 85%, 90%, 92%, 95%, 97%, 98%, or 99% sequence identity) thereto. In some embodiments the AAV capsid polypeptide, e.g., the AAV capsid variant, comprises an amino acid sequence comprising at least one, two, or three modifications, e.g., substitutions (e.g., conservative substitutions), but no more than 30, 20, or 10 modifications, e.g., substitutions (e.g., conservative substitutions), relative to the amino acid sequence of SEQ ID NO: 138. In some embodiments, the AAV capsid polypeptide, e.g., the AAV capsid variant, comprises an amino acid sequence encoded by the nucleotide sequence of SEQ ID NO: 137 or a nucleotide sequence substantially identical (e.g., having at least 70%, 75%, 80%, 85%, 90%, 92%, 95%, 97%, 98%, or 99% sequence identity) thereto. In some embodiments, the nucleotide sequence encoding the AAV capsid polypeptide, e.g., the AAV capsid variant, comprises the nucleotide sequence of SEQ ID NO: 137 or a nucleotide sequence substantially identical (e.g., having at least 70%, 75%, 80%, 85%, 90%, 92%, 95%, 97%, 98%, or 99% sequence identity) thereto. In some embodiments, the AAV capsid polypeptide, e.g., the AAV capsid variant, comprises substitution at position K449, e.g., a K449R substitution, numbered according to SEQ ID NO: 138.

In some embodiments, the AAV capsid polypeptide, e.g., the AAV capsid variant, comprises the amino acid sequence of SEQ ID NO: 11 or an amino acid sequence substantially identical (e.g., having at least 70%, 75%, 80%, 85%, 90%, 92%, 95%, 97%, 98%, or 99% sequence identity) thereto. In some embodiments the AAV capsid polypeptide, e.g., the AAV capsid variant, comprises an amino acid sequence comprising at least one, two, or three modifications, e.g., substitutions (e.g., conservative substitutions), but no more than 30, 20, or 10 modifications, e.g., substitutions (conservative substitutions), relative to the amino acid sequence of SEQ ID NO: 11, optionally wherein position 449 is not R.

In some embodiments, the capsid polypeptide, comprises the amino acid sequence of SEQ ID NO: 1 or an amino acid sequence substantially identical (e.g., having at least 70%, 75%, 80%, 85%, 90%, 92%, 95%, 97%, 98%, or 99% sequence identity) thereto. In some embodiments the AAV capsid polypeptide, e.g., the AAV capsid variant, comprises an amino acid sequence comprising at least one, two, or three modifications, e.g., substitutions (e.g., conservative substitutions), but no more than 30, 20, or 10 modifications, e.g., substitutions (e.g., conservative substitutions), relative to the amino acid sequence of SEQ ID NO: 1.

In some embodiments, the capsid polypeptide, comprises the amino acid sequence of SEQ ID NO: 10 or an amino acid sequence substantially identical (e.g., having at least 70%, 75%, 80%, 85%, 90%, 92%, 95%, 97%, 98%, or 99% sequence identity) thereto. In some embodiments the AAV capsid polypeptide, e.g., the AAV capsid variant, comprises an amino acid sequence comprising at least one, two, or three modifications, e.g., substitutions (e.g., conservative substitutions), but no more than 30, 20, or 10 modifications, e.g., substitutions (e.g., conservative substitutions), relative to the amino acid sequence of SEQ ID NO: 10. In some embodiments, the AAV capsid polypeptide, e.g., the AAV capsid variant, comprises a peptide comprising the amino acid sequence of TLA VPFK (SEQ ID NO: 1262). In some embodiments, the peptide is present immediately subsequent to position 588, relative to a reference sequence numbered according to SEQ ID NO: 138. In some embodiments, the capsid polypeptide comprises the amino acid substitutions of A587D and Q588G, numbered according to SEQ ID NO: 138.

In some embodiments, the AAV capsid polypeptide, e.g., the AAV capsid variant, comprises the amino acid substitution of K449R, numbered according to SEQ ID NO: 138; and a peptide comprising the amino acid sequence of TLAVPFK (SEQ ID NO: 1262), wherein the peptide is present immediately subsequent to position 588, relative to a reference sequence numbered according to SEQ ID NO: 138.

In some embodiments, the AAV capsid polypeptide, e.g., the AAV capsid variant, comprises the amino acid substitution of K449R, numbered according to SEQ ID NO: 138; an peptide comprising the amino acid sequence of TLAVPFK (SEQ ID NO: 1262), wherein the insert is present immediately subsequent to position 588, relative to a reference sequence numbered according to SEQ ID NO: 138; and the amino acid substitutions of A587D and Q588G, numbered according to SEQ ID NO: 138.

In some embodiments, the AAV capsid polypeptide, e.g., the AAV capsid variant, comprises a peptide comprising the amino acid sequence of TLAVPFK (SEQ ID NO: 1262), wherein the insert is present immediately subsequent to position 588, relative to a reference sequence numbered according to SEQ ID NO: 138; and the amino acid substitutions of A587D and Q588G, numbered according to SEQ ID NO: 138.

In some embodiments, the AAV capsid variant, comprises immediately subsequent to position 448, 449, 452, 453, 455, numbered relative to SEQ ID NO: 138 or corresponding to equivalent positions in any other AAV serotype (e.g., AAV1; AAV2; AAV3; AAV3b; AAV4; AAV5; AAV6; AAV7; AAV8; AAVrh8; AAVrh10; AAVrh32.33; AAVrh74; SEQ ID NOs: 1, 10, 11-14, 16, 18, 20, or 3636; PHP.N; PHP.B; or an AAV serotype as provided in Table 6 of WO 2021/230987 (the contents of which are hereby incorporated by reference in their entirety)), at least 3, 4, 5, 6, 7, 8, 9, 10, 11, 12, or 13 consecutive amino acids of any of amino acid sequence provided in Tables 1A, 2A, 2B, 21-23, 25, 26, or 30. In some embodiments, the at least 3, 4, 5, 6, 7, 8, 9, 10, 11, 12, or 13 consecutive amino acids of any of amino acid sequence provided in Tables 1A, 2A, 2B, 21-23, 25, 26, or 30 replaces at least one, two, three, four, five, six, seven, eight, nine, ten, or all of positions K449, T450, 1451, N452, G453, S454, G455, Q456, N457, Q458, and/or Q459, numbered according to SEQ ID NO: 138 or corresponding to equivalent positions in any other AAV serotype (e.g., AAV1; AAV2; AAV3; AAV3b; AAV4; AAV5; AAV6; AAV7; AAV8; AAVrh8; AAVrh10; AAVrh32.33; AAVrh74; SEQ ID NOs: 1, 10, 11-14, 16, 18, 20, or 3636; PHP.N; PHP.B; or an AAV serotype as provided in Table 6 of WO 2021/230987 (the contents of which are hereby incorporated by reference in their entirety). In some embodiments, the at least 3, 4, 5, 6, 7, 8, 9, 10, 11, 12, or 13 consecutive amino acids of any of amino acid sequence provided in Tables 1A, 2A, 2B, 21-23, 25, 26, or 30 replaces positions S454, G455, or both positions S454 and G455, numbered according to SEQ ID NO: 138 or corresponding to equivalent positions in any other AAV serotype (e.g., AAV1; AAV2; AAV3; AAV3b; AAV4; AAV5; AAV6; AAV7; AAV8; AAVrh8; AAVrh10; AAVrh32.33; AAVrh74; SEQ ID NOs: 1, 10, 11-14, 16, 18, 20, or 3636; PHP.N; PHP.B; or an AAV serotype as provided in Table 6 of WO 2021/230987 (the contents of which are hereby incorporated by reference in their entirety). In some embodiments, the AAV capsid variant comprises an amino acid other than the wild-type, e.g., native, amino acid, at one, two, three, four, five, six, seven, eight, nine or all of positions T450, I451, N452, G453, S454, G455, Q456, N457, Q458, and/or Q459, numbered according to SEQ ID NO: 138 or corresponding to equivalent positions in any other AAV serotype (e.g., AAV1; AAV2; AAV3; AAV3b; AAV4; AAV5; AAV6; AAV7; AAV8; AAVrh8; AAVrh10; AAVrh32.33; AAVrh74; SEQ ID NOs: 1, 10, 11-14, 16, 18, 20, or 3636; PHP.N; PHP.B; or an AAV serotype as provided in Table 6 of WO 2021/230987 (the contents of which are hereby incorporated by reference in their entirety). In some embodiments, the AAV capsid variant comprises an amino acid other than the wild-type, e.g., native, amino acid, at position S454, G455, or both positions S454 and G455, numbered according to SEQ ID NO: 138 or corresponding to equivalent positions in any other AAV serotype (e.g., AAV1; AAV2; AAV3; AAV3b; AAV4; AAV5; AAV6; AAV7; AAV8; AAVrh8; AAVrh10; AAVrh32.33; AAVrh74; SEQ ID NOs: 1, 10, 11-14, 16, 18, 20, or 3636; PHP.N; PHP.B; or an AAV serotype as provided in Table 6 of WO 2021/230987 (the contents of which are hereby incorporated by reference in their entirety)). In some embodiments, the AAV capsid variant comprises a modification, e.g., substitution, at one, two, three, four, five, six, seven, eight, nine, ten or all of positions K449, T450, 1451, N452, G453, S454, G455, Q456, N457, Q458, and/or Q459, numbered according to SEQ ID NO: 138 or corresponding to equivalent positions in any other AAV serotype (e.g., AAV1; AAV2; AAV3; AAV3b; AAV4; AAV5; AAV6; AAV7; AAV8; AAVrh8; AAVrh10; AAVrh32.33; AAVrh74; SEQ ID NOs: 1, 10, 11-14, 16, 18, 20, or 3636; PHP.N; PHP.B; or an AAV scrotype as provided in Table 6 of WO 2021/230987 (the contents of which are hereby incorporated by reference in their entirety). In some embodiments, the AAV capsid variant comprises a modification, e.g., substitution, at position S454, G455, or both positions S454 and G455, numbered according to SEQ ID NO: 138 or corresponding to equivalent positions in any other AAV serotype (e.g., AAV1; AAV2; AAV3; AAV3b; AAV4; AAV5; AAV6; AAV7; AAV8; AAVrh8; AAVrh10; AAVrh32.33; AAVrh74; SEQ ID NOs: 1, 10, 11-14, 16, 18, 20, or 3636; PHP.N; PHP.B; or an AAV serotype as provided in Table 6 of WO 2021/230987 (the contents of which are hereby incorporated by reference in their entirety).

In some embodiments, an AAV capsid variant, e.g., an AAV capsid variant described herein (e.g., an AAV capsid variant comprising a peptide described herein). In some embodiments, an AAV capsid variant comprises a peptide as set forth in any of Tables 1A, 2A, 2B, 21-23, 25, 26, or 30.

TABLE 1A
Exemplary Peptide Sequences

| | SEQ | | SEQ | | SEQ | | SEQ |
|---|---|---|---|---|---|---|---|
| Peptide | ID | Peptide | ID | Peptide | ID | Peptide | ID |
| Sequence | NO: | Sequence | NO: | Sequence | NO: | Sequence | NO: |
| GSGSPHSKAQN | 200 | GSLHHDNHGQN | 385 | GSVFGVPSG | 570 | GSIAMTSHG | 755 |
| QQT | | QQT | | QNQQT | | QNQQT | |
| GHDSPHKSGQN | 201 | GIMARDSSGQN | 386 | GSGLPDRNL | 571 | GSPGVSPSG | 756 |
| QQT | | QQT | | QNQQT | | QNQQT | |
| GSGSPHARMQN | 202 | GVVHITNSGQN | 387 | GSGTHNSAIQ | 572 | GSGQNQQTG | 757 |
| QQT | | QQT | | NQQT | | SSSRV | |
| GSGSPHVKSQN | 203 | GSGQNQHSAPF | 388 | GSGMIIASMQ | 573 | GSGQHLPLL | 758 |
| QQT | | NQT | | NQQT | | GNQQT | |
| GQDSPHKSGQN | 204 | GSGQTSGLKQN | 389 | GGITWTDSG | 574 | GSDHSHRGG | 759 |
| QQT | | QQT | | QNQQT | | QNQQT | |
| GSGSPHASRQN | 205 | GSGQNQQTSLS | 390 | GSGQNQQAS | 575 | GSGIVTKLG | 760 |
| QQT | | NTA | | GRQQT | | QNQQT | |
| GSGSPHASRQN | 206 | GSGQNQAVHNK | 391 | GSGQNQQPH | 576 | GSGQDVTKT | 761 |
| KQT | | SQT | | LKSLT | | GNQQT | |
| GSGSPHVKIQNQ | 207 | GVHTHLPSGQN | 392 | GPPQHMTSG | 577 | GSGQNQQSH | 762 |
| QT | | QQT | | QNQQT | | GRIGT | |
| GSGSPHSKAKN | 208 | GHLTMHNSGQN | 393 | GSGQNQQAS | 578 | GSGQNQQIN | 763 |
| QQT | | HQT | | LPSRT | | HRSPT | |
| GSGSPHKKNQN | 209 | GSGSSSRPYQNQ | 394 | GSGQIVSTQT | 579 | GSGDDSRVG | 764 |
| QQT | | QT | | NQQT | | QNQQT | |
| GSGSPHVRMQN | 210 | GILLATPSGQNQ | 395 | GSGKGHSAG | 580 | GSGQSTLKRI | 765 |
| QQT | | QT | | QNQQT | | NQQT | |
| GSGSPHASRQK | 211 | GSGQNAGSFPN | 396 | GSGQNTRLQ | 581 | GSGSQHSKA | 766 |
| QQT | | QQT | | LGQQT | | QNQQT | |
| GHSSPHRSGQN | 212 | GSRDGHTVGQN | 397 | GSVGSRPVG | 582 | GSGQNQQHA | 767 |
| QQT | | QQT | | QNQQT | | SSNNT | |
| GMRTYHLSGQN | 213 | GSLLISTSGQNQ | 398 | GSSHTLALG | 583 | GSRTYQVSG | 768 |
| QQT | | QT | | QNQQT | | QNQQT | |
| GSGSPHTRGQN | 214 | GSGAMPSHGQN | 399 | GMYEYSQSG | 584 | GSGQNQGLL | 769 |
| QQT | | QQT | | QNQQT | | SSPQT | |
| GSGIIPVSSQNQ | 215 | GALVSPISGQNQ | 400 | GNGQNQQHS | 585 | GSGGGLQHN | 770 |
| QT | | QT | | ILHGT | | QNQQT | |
| GSEYGHKSGQN | 216 | GSLSSHGVGQN | 401 | GSGYNQPHL | 586 | GSGQNQQTT | 771 |
| QQT | | QQT | | QNQQT | | AATRM | |
| GRGQNVSSVHR | 217 | GSGQNQQASLA | 402 | GPLVNASSG | 587 | GSGQNQRAS | 772 |
| QQT | | MRT | | QNQQT | | ILVQT | |
| GSSHRFYGGQN | 218 | GPGLGSHSGQN | 403 | GSGQNQQVL | 588 | GSGQNLGLL | 773 |
| QQT | | QQT | | TTART | | GAQQT | |
| GYFVAAWSGQN | 219 | GHDSQHKSGQN | 404 | GSGQNQHSV | 589 | GSLDLGRSG | 774 |
| QQT | | QQT | | HNDQT | | QNQQT | |
| GSVLHSHAAQN | 220 | GSGLTLSATQN | 405 | GAGLIMHSG | 590 | GNSQVKVSG | 775 |
| QQT | | QQT | | QNQQT | | QNQQT | |
| GSGDLVVSTQN | 221 | GSGQVVAHVGN | 406 | GMGRHSASG | 591 | GSSGSHQYG | 776 |
| QQT | | QQT | | QNQQT | | QNQQT | |
| GSYGMAASGQN | 222 | GSGLRTMTTQN | 407 | GSHSQSGHG | 592 | GSGQNQQQR | 777 |
| QQT | | QQT | | QNQQT | | DGTLT | |
| GLNHFGASGQN | 223 | GSGQVGRLLQN | 408 | GSSTTIVSGQ | 593 | GRGQHVSVA | 778 |
| QQT | | QQT | | NHQT | | NNQQT | |
| GSTGSHSAGQN | 224 | GSGQLSHQSVN | 409 | GRHLVTASG | 594 | GDSSSRISGQ | 779 |
| QHT | | QQT | | QNQQT | | NQQT | |
| GLAGHTVSGQN | 225 | GSGDRYQTLQN | 410 | GSGQNQQHA | 595 | GSGQNQQHS | 780 |
| QQT | | QQT | | NLNQT | | LSSQT | |
| GIILGASSGQNQ | 226 | GSGQNQQLKSS | 411 | GSGSTHSKA | 596 | GSLMDVHRG | 781 |
| QT | | AQT | | QNQQT | | QNQQT | |
| GSGVSTYNIQNQ | 227 | GSGQNQYSIPVA | 412 | GSGQNKQML | 597 | GSIQYQSSGQ | 782 |
| QT | | QT | | SGNTT | | NQQT | |
| GSLVSVQTGQN | 228 | GSGERLHLTQN | 413 | GSGQVHNPT | 598 | GLGSKNPSG | 783 |
| QQT | | QQT | | QNQQT | | QNQQT | |
| GQSSPHRSGQN | 229 | GSGHNQQVRTA | 414 | GSGQNQQIP | 599 | GSGQLVLTL | 784 |
| QQT | | PNT | | HVHQT | | QNQQT | |
| GREYGHKSGQN | 230 | GGLSHVMSGQN | 415 | GSLHAGLSG | 600 | GSGQNQQTS | 785 |
| QQT | | QQT | | QNQQT | | QPLPG | |
| GHTLTLSSGQN | 231 | GSGQSHRDVLN | 416 | GPAQHGTSG | 601 | GSGQNQQNL | 786 |
| QQT | | QQT | | QNQQT | | GKLNT | |
| GSITLIPSGQNQ | 232 | GSGQNLAGRMD | 417 | GEKAVTSSG | 602 | GTTAHQPSG | 787 |
| QT | | QQT | | QNQQT | | QNQQT | |
| GSNGFTALGQN | 233 | GSGQNQQTNRG | 418 | GSGQNQQTM | 603 | GSGQNRAQI | 788 |
| QQT | | NPM | | ANGQR | | GTQQT | |
| GSGHSSHSVQN | 234 | GSGQSYQRDHN | 419 | GSGSPHSKD | 604 | GSGQYVHVS | 789 |
| QQT | | QQT | | QNQQT | | SNQQT | |
| GSGIPQRSGKNQ | 235 | GSLLSAGMGQN | 420 | GSFSMGYGG | 605 | GSGQNQQTA | 790 |
| QT | | QHT | | QNQQT | | HAFNI | |
| GSGDTLHMLQN | 236 | GSGQNQQTAIY | 421 | GSGTHLVSL | 606 | GSGQNQRTM | 791 |
| QQT | | RNI | | QNQQT | | VATQT | |
| GERHTVLSGQN | 237 | GSGQNQQTSGT | 422 | GSGQMQPHV | 607 | GSGQNPIRG | 792 |
| QQT | | TNC | | QNQQT | | AMQQT | |
| GSGMPQSHIQN | 238 | GMTSHSVSGQN | 423 | GSGQNQQVA | 608 | GSGYVITGS | 793 |
| QQT | | QQT | | GLNNT | | QNQQT | |
| GSGQLSGIGGNQ | 239 | GSSQSTGYQPN | 424 | GSSQNQQHD | 609 | GRGPKQSNI | 794 |
| QT | | QQT | | MRLRT | | QNQQT | |
| GSGQNRKPASF | 240 | GSLKPTTLGQN | 425 | GPASLPISGQ | 610 | GSGQNQQT | 795 |
| AQT | | QQT | | NQQT | | MLGKPC | |
| GSGSVSQLGQN | 241 | GRMFSLGSGQN | 426 | GSGQNQQPP | 611 | GSGQNQQVG | 796 |
| QQT | | QQT | | LATRT | | STVRT | |
| GSDFLGTHGQN | 242 | GSGQNQQTALG | 427 | GSSRVPVSG | 612 | GNVTTQKSG | 797 |
| QQT | | VKC | | QNQQT | | QNQQT | |
| GQIVQNPSGQN | 243 | GAMVSHSSGQN | 428 | GSGQNQQTN | 613 | GSGNPVSHL | 798 |
| QQT | | QQT | | LGHTT | | QNQQT | |
| GSGTQIPSQQNQ | 244 | GSGQNQQRNSD | 429 | GSGQNQQLV | 614 | GSLSHMESG | 799 |
| QT | | SVT | | SRVQT | | QNQQT | |
| GSGQNQQSARE | 245 | GSGQSMTLHLN | 430 | GPNSYPVSG | 615 | GRAPTNLSG | 800 |
| GLT | | QQT | | QKQQT | | QNQQT | |
| GSGLGMSTGQN | 246 | GSGQVHQAEVN | 431 | GHAHYQASG | 616 | GSGQNQQTV | 801 |
| QQT | | QQT | | QNQQT | | MTARA | |
| GSGLPVLSGQN | 247 | GSGQNQSQNHL | 432 | GSGQALLST | 617 | GSGMPASRL | 802 |
| QQT | | QQT | | GNQQT | | QNQQT | |
| GSGHSIRTDQNQ | 248 | GSLLTTASGQN | 433 | GSGQLPRQM | 618 | GVVRNHQSG | 803 |
| QT | | QQT | | TNQQT | | QNQQT | |
| GSGQSVQTVVN | 249 | GSGLIRTAAQNQ | 434 | GSGFPKSTEQ | 619 | GSGQNQHSV | 804 |
| QQT | | QT | | NQQT | | QVRQT | |
| GSGQNRAQSRF | 250 | GSGQNQQTVSR | 435 | GSRETSLSGQ | 620 | GSGQNTGHL | 805 |
| QQT | | QST | | NQQT | | TMQQT | |
| GGGDLGRSSQN | 251 | GSGQYANHGIN | 436 | GSGQNQQGT | 621 | GSGQNQQYA | 806 |
| QQT | | QQT | | GVSHT | | GKILT | |
| GGGTKMDSGQN | 252 | GSRSTGPSGQN | 437 | GSRTVPVYG | 622 | GSGNPHVRN | 807 |
| QQT | | QQT | | QNQQT | | QNQQT | |
| GSGSPHPSRQNQ | 253 | GRGVQQKLQQN | 438 | GSNAQSAHG | 623 | GSGQNGGSS | 808 |
| QT | | QQT | | QNQQT | | NRQQT | |
| GSGQFTNAGMN | 254 | GSGQNQQVHLS | 439 | GAFHLAASG | 624 | GSGQRLSQG | 809 |
| QQT | | TGT | | QNQQT | | VNHQT | |
| GGRNGHTVGQN | 255 | GSGQNQQLSAK | 440 | GSGQYRSSS | 625 | GSGQNAHAK | 810 |
| QQT | | SST | | DNQQT | | EGQQT | |
| GSGFGPQTGQN | 256 | GSGYKAARPQN | 441 | GSGQVYISTP | 626 | GSSPAPNSG | 811 |
| QQT | | QQT | | NQQT | | QNQQT | |
| GRTDSHTSGQN | 257 | GSAGISPSGQNQ | 442 | GSGVSTQLL | 627 | GLAHKTSSG | 812 |
| QQT | | QT | | QNQQT | | QNQQT | |
| GYEVLGSSGQN | 258 | GSGQNRAHAFL | 443 | GSGQLGLSV | 628 | GSGQNQQTP | 813 |
| QQT | | QQT | | TNQQT | | GAHKT | |
| GSVHLSVTGQN | 259 | GSGLSGITMQN | 444 | GSGSNMRLS | 629 | GSGQNQQSL | 814 |
| QQT | | QQT | | QNQQT | | SGSFT | |
| GFMSYKGSGQN | 260 | GPGSAHSSGQN | 445 | GSGQNLHSG | 630 | GSGQNQQST | 815 |
| QQT | | QQT | | LPQQT | | GTSRT | |
| GNIAGSVSGQN | 261 | GSSHTQALGQN | 446 | GSSHTLALG | 631 | GSGQNQQTV | 816 |
| QQT | | QQT | | QNKQT | | QSNLV | |
| GSGSHRDVSQN | 262 | GSGVHGVSSQN | 447 | GSGQNQHSL | 632 | GSGQNQQLG | 817 |
| QQT | | QQT | | PAHRT | | SRQCT | |
| GGLGSMSSGQN | 263 | GSSGRDMGGQN | 448 | GSGQNQGTV | 633 | GSGQNQYLR | 818 |
| QQT | | QQT | | YPNQT | | LELQT | |
| GSGHLPQSAQN | 264 | GERAFPTSGQN | 449 | GSGQNQQPS | 634 | GSGQNQQTS | 819 |
| QQT | | QQT | | LRQST | | PRLQT | |
| GGVLVGGSGQN | 265 | GGRIVSLSGQNQ | 450 | GSGQNARLK | 635 | GSGQNQQTT | 820 |
| QQT | | QT | | DNQQT | | SSNMT | |
| GTHPYTSSGQN | 266 | GSGQNSYSHTS | 451 | GHAGSTGSG | 636 | GTASTYNSG | 821 |
| QQT | | QQT | | QNQQT | | QNQQT | |
| GSGQNQQLKEN | 267 | GLGYPGSSGQN | 452 | GSGQALSSSG | 637 | GSGQNQQT | 822 |
| RST | | QQT | | NQQT | | MPQHKI | |
| GSGQNQQTSPH | 268 | GSGPQSHTGQN | 453 | GSGASESHR | 638 | GSGQSHLHT | 823 |
| NHT | | QQT | | QNQQT | | GNQQT | |
| GSGTLYPQSQN | 269 | GSGQNQQLSRD | 454 | GVGVITSSGQ | 639 | GVKGVGHSG | 824 |
| QQT | | AST | | NQQT | | QNQQT | |
| GSGQNQQSNWI | 270 | GSGQILHSVPNQ | 455 | GSLYGQSLG | 640 | GSGKVTKQS | 825 |
| TKT | | QT | | QNQQT | | QNQQT | |
| GSGYTSLFLQNQ | 271 | GSGFHTDSRQN | 456 | GSGQMSDVH | 641 | GSGQNQQTA | 826 |
| QT | | QQT | | GNQQT | | LEKSL | |
| GSGVMTHVLQN | 272 | GSGQSHSLATN | 457 | GSGQNQQHS | 642 | GSGYKDTYG | 827 |
| QQT | | QQT | | SKATT | | QNQQT | |
| GSVSDVRAGQN | 273 | GSGQNQQTLSK | 458 | GSGQNQQTS | 643 | GSGQNQQSG | 828 |
| QQT | | PWT | | VSQQT | | TFLST | |
| GSGQSHMATLN | 274 | GSGHAAISQQN | 459 | GSGQKMWK | 644 | GSGQNTGQH | 829 |
| QQT | | QQT | | LDNQQT | | MMQQT | |
| GSGLSVHLAQN | 275 | GSGQNQQQIGG | 460 | GSGQNVSMQ | 645 | GSGKNQQRP | 830 |
| QQT | | NST | | VNQQT | | GLDQT | |
| GSGLSHATQQN | 276 | GGGPMAGSGQN | 461 | GSGQNQRAT | 646 | GSGQSREISL | 831 |
| QQT | | QQT | | LSNQT | | NQQT | |
| GSGLSVQSGQN | 277 | GMRMEYQSGQ | 462 | GSGQASSKS | 647 | GTPTSPSSGQ | 832 |
| QQT | | NQQT | | ANQQT | | NQQT | |
| GSGHMTYREKN | 278 | GSGQNQQGTLL | 463 | GSGKNQTPIP | 648 | GKPAGGLSG | 833 |
| QQT | | HQT | | KGQT | | QNQQT | |
| GSKGVPTPGQN | 279 | GSGQNQRSSGG | 464 | GSGQNQQTR | 649 | GSGQNHRSA | 834 |
| QQT | | VQT | | QEGST | | DMQQT | |
| GSGLLPLSSQNQ | 280 | GSGQNQRGALA | 465 | GASSLATSG | 650 | GSGQNQQTL | 835 |
| QT | | TQT | | QNQQT | | PSLSL | |
| GNGLYAVSGQN | 281 | GSGTVHAATQN | 466 | GSGQRGSLT | 651 | GSPYMGATG | 836 |
| QQT | | QQT | | ENQQT | | QNQQT | |
| GFNGSPSSGQNQ | 282 | GSRMTQQFGQN | 467 | GSEQTRQRG | 652 | GSGHAKAVG | 837 |
| QT | | QQT | | QNQQT | | QNQQT | |
| GSGQIRHSDQN | 283 | GSSSPGASGQN | 468 | GSGQNQQTL | 653 | GHMKGVTS | 838 |
| QQT | | QQT | | TASKE | | GQNQQT | |
| GGQVAPSSGQN | 284 | GHPSPHVSGQN | 469 | GSGTSGKTG | 654 | GSGQNQKIL | 839 |
| QQT | | QQT | | KNQQT | | TLDQT | |
| GSFSMHTHGQN | 285 | GSGSHHASRQN | 470 | GQLVTFTSG | 655 | GSGQNQQTK | 840 |
| QQT | | QQT | | QNQQT | | VGHSA | |
| GSGQNQQVIQG | 286 | GAVGHSYSGQN | 471 | GSGQNQQSA | 656 | GIARTTISGQ | 841 |
| SNT | | QQT | | NKILT | | NQQT | |
| GRVLHSHAGQN | 287 | GSRSQYDIGQN | 472 | GSGQNQQHH | 657 | GSGQNQQTS | 842 |
| QQT | | QQT | | SSHTT | | VGFRT | |
| GSGQNQQTSLQ | 288 | GSGQGPQERGN | 473 | GSGQNQKG | 658 | GSGQNQQT | 843 |
| DQT | | QQT | | MQPNQT | | MIANIR | |
| GSGLGRAPVQN | 289 | GSIAHVGTGQN | 474 | GSGQLVSGL | 659 | GDMTRSSSG | 844 |
| QQT | | QQT | | YNQQT | | QNQQT | |
| GNGFSSASGQN | 290 | GSGQNQQKQNH | 475 | GSSVGVPSG | 660 | GSGHMSDLR | 845 |
| QQT | | GNT | | QNQQT | | QNQQT | |
| GSGQMASRESN | 291 | GSGQNQQALGS | 476 | GSGQNQQW | 661 | GRGAVMAS | 846 |
| QQT | | QRT | | DSRRQT | | GQNQQT | |
| GPGLPNHSGQN | 292 | GSGAITHMPQN | 477 | GSEQTRQSG | 662 | GSGQNQQLS | 847 |
| QQT | | QQT | | QNQQT | | GKSVT | |
| GNIQWQGSGQN | 293 | GSGQRNPLLLN | 478 | GSGIGSHIPQ | 663 | GSHTLVVSG | 848 |
| QQT | | QQT | | NQQT | | QNQQT | |
| GMSAHMSSGQN | 294 | GSSGIPVSHQNQ | 479 | GSGQNQRLH | 664 | GSGPWSAGL | 849 |
| QQT | | QT | | GVDQT | | QNQQT | |
| GHSFVNRSGQN | 295 | GVHSLTPSGQN | 480 | GEVSRVLSG | 665 | GSGQHSPHA | 850 |
| QQT | | QQT | | QNQQT | | LNQQT | |
| GRAVMDHSGQ | 296 | GVIVLHGSGQN | 481 | GSGQNQQKV | 666 | GSGQNQQPN | 851 |
| NQQT | | QQT | | SPLLT | | SGSMT | |
| GALTVMQSGQN | 297 | GGTRVVDSGQN | 482 | GSGLALERS | 667 | GSGLAHLGG | 852 |
| QQT | | QQT | | QNQQT | | QNQQT | |
| GSGQRSPVLPNQ | 298 | GSGGVTYQSQN | 483 | GPDRIGSSGQ | 668 | GSSVRYEPK | 853 |
| QT | | QQT | | NQQT | | QNQQT | |
| GSGQNGHLSLK | 299 | GSGQNQAGHGP | 484 | GSGQNQDHQ | 669 | GSGQNQQAR | 854 |
| QQT | | GQT | | NKQQT | | PLELT | |
| GSLPRGTSDQN | 300 | GSGQLVTSGPN | 485 | GSGQNQQTA | 670 | GSGQPRSTGI | 855 |
| QQT | | QQT | | LYNNT | | NQQT | |
| GVAGSLVSGQN | 301 | GSGIAAQRTQN | 486 | GSGAVHLTA | 671 | GSGQNQAN | 856 |
| QQT | | QQT | | QNQQT | | WVKVQT | |
| GRGGIPQSGQN | 302 | GSTPAGVGGQN | 487 | GSLVSTQSG | 672 | GSGHLFQSG | 857 |
| QQT | | QQT | | QNQQT | | QNQQT | |
| GSGQYASSIPNQ | 303 | GSGQNQQTSTG | 488 | GSGVSARMV | 673 | GSGQNRGISI | 858 |
| QT | | VHS | | QNQQT | | SQQT | |
| GTDFGRQSSQN | 304 | GSGQIRQLVDN | 489 | GSGQTRMPL | 674 | GSGTHYDNR | 859 |
| QQT | | QQT | | ANQQT | | QNQQT | |
| GIFMQTPSGQNQ | 305 | GSLIGMQSGQN | 490 | GSGISSRNMQ | 675 | GSGQNQQTS | 860 |
| QT | | QQT | | NQQT | | TTPLP | |
| GSGQNQQTRLV | 306 | GSGQIKGKMDN | 491 | GSGEKVHSG | 676 | GSGQVHASQ | 861 |
| DLT | | QQT | | QNQQT | | VNQKT | |
| GTREMPLSGQN | 307 | GSGSDMSSWQN | 492 | GSGQNQQKL | 677 | GSSGHRESG | 862 |
| QQT | | QQT | | SSMST | | QNQQT | |
| GSRLVHVHGQN | 308 | GRGQNQQHTGL | 493 | GSGQNQQTG | 678 | GLSAEKSSG | 863 |
| QQT | | ATT | | QHMRV | | QNQQT | |
| GSGRLVPNGPN | 309 | GSGQNQQTLYS | 494 | GSGMIHTTA | 679 | GSGQEHRSL | 864 |
| QQT | | SNT | | QNQQT | | ANQQT | |
| GSGYLRESPQN | 310 | GSGQTQVLKSN | 495 | GSGQNWPAL | 680 | GSGQTVVRI | 865 |
| QQT | | QQT | | KGQQT | | ANQQT | |
| GARIQNASGQK | 311 | GSRTLSNVGQN | 496 | GASHMSISG | 681 | GSGQNVSSV | 866 |
| QQT | | QQT | | QNQQT | | HRQQT | |
| GLSNPMPSGQN | 312 | GSGVQHSLPQN | 497 | GSDQNQQLG | 682 | GSGASRMSI | 867 |
| QQT | | QQT | | YSKQT | | QNQQT | |
| GSTVQDTRGQN | 313 | GNYLHQASGQN | 498 | GIPSIRESGQ | 683 | GVAFIGSSGQ | 868 |
| QQT | | QQT | | NQQT | | NQQT | |
| GPFGMPSSGQN | 314 | GSGGTSVHQQN | 499 | GSGIPSVKFQ | 684 | GSGQNQQTV | 869 |
| QQT | | QQT | | NQQT | | PTRQT | |
| GSGQNHGVLSN | 315 | GMDHSRPSGQN | 500 | GSGQNQQTS | 685 | GSGQAAKSS | 870 |
| QQT | | QQT | | VSQNV | | QNQQT | |
| GSGYSMSQAQN | 316 | GSGQNQQSMGT | 501 | GSGQNQQIG | 686 | GSGQNQQVA | 871 |
| QQT | | FTT | | ESRMT | | IRTST | |
| GSGMLTHTLQN | 317 | GSGQNQQTPLR | 502 | GSGSSSMSFQ | 687 | GSVHMQNA | 872 |
| QQT | | PPT | | NQQT | | GQNQQT | |
| GRGSPHASRQN | 318 | GSGQNQHHSVS | 503 | GSGQKQERA | 688 | GSGMRQAG | 873 |
| QQT | | QQT | | VSKQT | | VQNQQT | |
| GLSWPSTSGQN | 319 | GSGQLRSLSTNQ | 504 | GCTTRLNSG | 689 | GSGQNQQVG | 874 |
| QQT | | QT | | QNQQT | | GKTVT | |
| GNSMERTSGQN | 320 | GSGSPRQLSQN | 505 | GSGQNQQIIS | 690 | GVHDMRVS | 875 |
| QQT | | QQT | | TKIT | | GQNQQT | |
| GSGMSPSTLQN | 321 | GSGQNQQTTAS | 506 | GSGQNQQKS | 691 | GSGQHVSVA | 876 |
| QQT | | SHT | | LNGNT | | NNQQT | |
| GSGHGQVLSQN | 322 | GRGQVVSTHQN | 507 | GSGIPAPRLQ | 692 | GSAAMSVRG | 877 |
| QQT | | QQT | | NQQT | | QNQQT | |
| GRGQIYSTGGN | 323 | GSAQVSMVGQN | 508 | GSGQIRESM | 693 | GVSRGGPSG | 878 |
| QQT | | QQT | | GNQQT | | QNQQT | |
| GVVAAHNSGQN | 324 | GSSTLVTIGKNQ | 509 | GSGQNSGVH | 694 | GSGQMVHTI | 879 |
| QQT | | QT | | FNQQT | | GNQQT | |
| GDSSLRHSGQN | 325 | GFAHQASSGQN | 510 | GSGQNIGHSL | 695 | GRGGSMAET | 880 |
| QQT | | QQT | | PQQT | | QNQQT | |
| GSLVSQGAGQN | 326 | GSGQPVLSISNQ | 511 | GSGERSISVQ | 696 | GSGHTNPTR | 881 |
| QQT | | QT | | NQQT | | QNQQT | |
| GSLLQAHSGQN | 327 | GSGQSHRSELN | 512 | GSGLKPNVL | 697 | GSGEAARYE | 882 |
| QQT | | QQT | | QNQQT | | QNQQT | |
| GSGHIYVGIQNQ | 328 | GSSVGSPIGQNQ | 513 | GSGQVAYAQ | 698 | GSGQNERHL | 883 |
| QT | | QT | | GNQQT | | VLQQT | |
| GHHTTVQSGQN | 329 | GSGMPIRNVQN | 514 | GSGQSSYGS | 699 | GSGQNQQSK | 884 |
| QQT | | QQT | | GNQQT | | QQVLT | |
| GSRQSKRNELN | 330 | GSSTRVDSGQN | 515 | GSGQNQAMT | 700 | GSGQARAHR | 885 |
| QQT | | QQT | | HGDQT | | GNQQT | |
| GSGQNQQHVSS | 331 | GSGQNQQTAMR | 516 | GSGQNQALV | 701 | GSGQNQQPL | 886 |
| PRT | | STT | | SMGQT | | DTSRT | |
| GSSKELLWGQN | 332 | GSGQNQQHSSS | 517 | GSGQNPSFM | 702 | GSGQNQQLA | 887 |
| QQT | | HLT | | RGQQT | | NMVTT | |
| GSLSTPSSGQNQ | 333 | GSRNGHAVGQN | 518 | GSGQNQQSH | 703 | GSGQMKDL | 888 |
| QT | | QQT | | LRTNT | | HRNQQT | |
| GSIGYAGQGQN | 334 | GLGAYQSSGQN | 519 | GYTRLETSG | 704 | GSGQNQHLS | 889 |
| QQT | | QQT | | QNQQT | | SFVQT | |
| GSGQNQRVSNS | 335 | GPGLSGHSGQN | 520 | GSGQSYDMR | 705 | GSGQNQQPS | 890 |
| QQT | | QQT | | GNQQT | | SRVTT | |
| GSGYASHVQQN | 336 | GSTGIVSSGQNQ | 521 | GSRTTQDIGQ | 706 | GSGQNQQLA | 891 |
| QQT | | QT | | NQQT | | ITLGT | |
| GSGEYSRSGQN | 337 | GSRTTQVIGQNQ | 522 | GSGHPYKAA | 707 | GSGQNQQTV | 892 |
| QQT | | QT | | QNQQT | | GNPAT | |
| GSVSTHSSGQN | 338 | GSGLLHRAQQN | 523 | GRLSNAHGG | 708 | GSGQNQGRA | 893 |
| QQT | | QQT | | QNQQT | | HPMQT | |
| GSGQNQHSLGN | 339 | GSGQNAQQAAA | 524 | GSGQNQRAV | 709 | GSGQLIASV | 894 |
| YQT | | QQT | | LNDQT | | VNQQT | |
| GSGGLDTRGQN | 340 | GSGQNQQSALR | 525 | GGSHTYGGG | 710 | GSSVRSLVG | 895 |
| QQT | | TQT | | QNQQT | | QNQQT | |
| GNILHATSGQN | 341 | GSGFLSDTRQN | 526 | GSSVNSMIG | 711 | GGAGSAHSG | 896 |
| QQT | | QQT | | QNQQT | | QNQQT | |
| GSGQSYTMTQN | 342 | GSGLLYHDQQN | 527 | GNSSMMGSG | 712 | GSDQNQQT | 897 |
| QQT | | QQT | | QNQQT | | MSSTRT | |
| GSGQNQHSAPN | 343 | GSGQNQHYSLH | 528 | GNRDRPSSG | 713 | GSGQNQQM | 898 |
| SQT | | KQT | | QNQQT | | AGAFRT | |
| GSGQNQQTMDH | 344 | GSGHSPLPQQN | 529 | GSGNMHASR | 714 | GSLGNLQRG | 899 |
| NRT | | QQT | | QNQQT | | QNQQT | |
| GSNGGVGTGQN | 345 | GNGHSMRPNQN | 530 | GFIFPKVSGQ | 715 | GSGPSISHGQ | 900 |
| QQT | | QQT | | NQQT | | NQQT | |
| GAGSIIPSGQNQ | 346 | GSGLKWSTLQN | 531 | GSGQNQQLK | 716 | GSGQNQQSS | 901 |
| QT | | QQT | | NSTST | | FNVQT | |
| GSGQTHGGQHN | 347 | GSGQMGRQAV | 532 | GSGQNQQSQ | 717 | GSGQNQQTG | 902 |
| QQT | | NQQT | | YMPRT | | QATHN | |
| GSNLSFQSGQN | 348 | GSGQNQQTSGV | 533 | GSGQRMADI | 718 | VSGSPHSKA | 903 |
| QQT | | LTL | | GNQQT | | QNQQT | |
| GATLQVHSGQN | 349 | GSGQNQQALHN | 534 | GSGQNQSHY | 719 | CSGSPHSKA | 904 |
| QQT | | PHT | | PSQQT | | QNQQT | |
| GSGFNQRSEQN | 350 | GSGQNQQVIPNS | 535 | GSDGKMHRG | 720 | GSGSPHRKA | 905 |
| QQT | | KT | | QNQQT | | QNQQT | |
| GSGSLRDFDQN | 351 | GSPLQDRVGQN | 536 | GSGSVGFIGQ | 721 | GRGSPHSKA | 906 |
| QQT | | QQT | | NQQT | | QNQQT | |
| GSGDSITGKQNQ | 352 | GSGQNQYSSTN | 537 | GLHGMTLSG | 722 | GSGSPHSKA | 907 |
| QT | | PQT | | QNQQT | | QNKQT | |
| GSGQDRNIVQN | 353 | GAMTVTISGQN | 538 | GSDQSKRGD | 723 | GSGSPHSKA | 908 |
| QQT | | QQT | | SNQQT | | QTQQT | |
| GSGLSHSHQQN | 354 | GSGQNQQLQTLI | 539 | GSLFLATGG | 724 | GSGSTHASR | 909 |
| QQT | | RT | | QNQQT | | QNQQT | |
| GSGQNQQTGMS | 355 | GSGLRQTSQQN | 540 | GSGQNQQPS | 725 | GSGSPHKYG | 910 |
| SVK | | QQT | | AFSKT | | QNQQT | |
| GSVTHGISGQNQ | 356 | GSGQNQQTGLR | 541 | GSGQLPQSG | 726 | GSGSPHKFG | 911 |
| QT | | QQT | | LNQQT | | QNQQT | |
| GVVAHQPSGQN | 357 | GSGQTRQMKDN | 542 | GSGSKQNAL | 727 | VSGSPHKFG | 912 |
| QQT | | QQT | | QNQQT | | QNQQT | |
| GSGPILGQLQNQ | 358 | GSGQNHGLQSG | 543 | GSGQRRELS | 728 | GSGSPHSKA | 913 |
| QT | | QQT | | QNQQT | | QNHQT | |
| GSGHVPNSGLN | 359 | GSGQSHRQPEN | 544 | GSGQREPKA | 729 | GSGSPHSKA | 914 |
| QQT | | QQT | | SNQQT | | QHQQT | |
| GDAGVRSSGQN | 360 | GSGQDRHIVQN | 545 | GSGQNQQHP | 730 | GSGSPHKTY | 915 |
| QQT | | QQT | | STQQT | | QNQQT | |
| GSGSQLMSLQN | 361 | GSGQNQQLPHS | 546 | GSQSTLGLG | 731 | VSGSPHASR | 916 |
| QQT | | NLT | | QNQQT | | QNQQT | |
| GSGLDYSQRQN | 362 | GSGQLSVPYDN | 547 | GSGQNQQMP | 732 | GSGSPHKFG | 917 |
| QQT | | QQT | | GLSST | | KNQQT | |
| GSGQSSGRLINK | 363 | GSGRNPQTQPL | 548 | GSGQNQQTV | 733 | GSGSPHASR | 918 |
| QT | | QQT | | GGKNL | | QNQHT | |
| GSSVSPSSGQNQ | 364 | GSGQPYSTGLN | 549 | GSSREFHSGQ | 734 | GSHSPHKSG | 919 |
| QT | | QQT | | NQQT | | QNQQT | |
| GSGQVVGLSGN | 365 | GSGQNQQTHGG | 550 | GSGQNQQTV | 735 | GSGQNQQRR | 920 |
| QQT | | LRD | | PSNLV | | MSPST | |
| GSNMGVPLGQN | 366 | GAYGMVSSGQN | 551 | GSGQNAYSS | 736 | GSGSPHSKP | 921 |
| QQT | | QQT | | QAQQT | | QNQQT | |
| GSFYPSSTGQNQ | 367 | GSGIQSSYSQNQ | 552 | GSGQNKDHS | 737 | GSGSPHKFG | 922 |
| QT | | QT | | TRRQT | | QKQQT | |
| GSGQNQQTRLT | 368 | GPRLSDQSGQN | 553 | GQLGSVGSG | 738 | VSGSPHGAR | 923 |
| DLT | | QQT | | QDQQT | | QNQQT | |
| GPTNGRSSGQN | 369 | GSGQNQQTHPS | 554 | GSGQHAAPG | 739 | GSGSPHSKA | 924 |
| QQT | | PCT | | HNQQT | | QKQQT | |
| GSGLLHGKLQN | 370 | GSGQSFQMHTN | 555 | GSGQNQQTS | 740 | GSHSPHKRG | 925 |
| QQT | | QQT | | QSPPT | | QNQQT | |
| GANMGHVSGQ | 371 | GSGQNQQTGNP | 556 | GSGNYRDHE | 741 | GSGQNRQRL | 926 |
| NQQT | | KHT | | QNQQT | | KGLET | |
| GSGQNQQSGRG | 372 | GFSSAVHSGQN | 557 | GSGQHSNQH | 742 | GSGSPHKLG | 927 |
| DLT | | QQT | | VNQQT | | QNQQT | |
| GSHGHYASGQK | 373 | GSGQNQQTSMS | 558 | GSGQTARNG | 743 | GSGSPHKTS | 928 |
| QQT | | NAT | | INQQT | | KNQQT | |
| GSGDLRISPQNQ | 374 | GSGQDMKQHH | 559 | GSGQNQQHY | 744 | GSGSPHKIGQ | 929 |
| QT | | NQQT | | GSQGT | | NQQT | |
| GSGMPVILGQN | 375 | GLRLSTPSGQNQ | 560 | GSGSPQASR | 745 | GSGQDSPHV | 930 |
| QQT | | QT | | QNQQT | | RNQQT | |
| GRGVITSSGQNH | 376 | GSGQNQQTSVY | 561 | GSGFSHSMG | 746 | GSGSPHKTS | 931 |
| QT | | MNT | | KNQQT | | QNQQT | |
| GSGHSVSGPQN | 377 | GSGQNQYSQSS | 562 | GSGQSHSLET | 747 | GSGSPHASR | 932 |
| QQT | | MQT | | NQQT | | KNQQT | |
| GSRNGHTVGRN | 378 | GSGQNQQSMAD | 563 | GTEQTRQSG | 748 | GSHSPHKSG | 933 |
| QQT | | HTT | | QNQQT | | QKQQT | |
| GAGVHMVSGQ | 379 | GWERSFVSGQN | 564 | GSGRHLASV | 749 | GSGSPHKTS | 934 |
| NQQT | | QQT | | QNQQT | | QKQQT | |
| GSGQNHRPSVL | 380 | GLLAGKSSGQN | 565 | GLGSKNHSG | 750 | GSGSPHVRG | 935 |
| QQT | | QQT | | QNQQT | | QNKQT | |
| GSGSPRDSIQNQ | 381 | GKSFVPQSGQN | 566 | GSGQNQQTS | 751 | GSGSPHKTT | 936 |
| QT | | QQT | | HFPSA | | QNQQT | |
| GSGQGIHSSVNQ | 382 | GSGQMQSAGSN | 567 | GSGQLSGTP | 752 | GSGPVRALR | 937 |
| QT | | QQT | | QNQQT | | QNQQT | |
| GSGQQLSITPNQ | 383 | GSDQNQRLTSS | 568 | GSGQNQQAP | 753 | GSGSPHVRG | 938 |
| QT | | MQT | | HKKET | | QKQQT | |
| GGYHSQTSGQN | 384 | GESRAVLSGQN | 569 | GSGQNQQTL | 754 | CSGSPHKTS | 939 |
| QQT | | QQT | | RGSLE | | QNQQT | |
| CSHSPHKSGQN | 940 | DAGSPHSKAQN | 1909 | GSGSPHASR | 2019 | GNDSPHKSV | 2129 |
| QQT | | QQ | | QNQQ | | QNQQ | |
| GHDSPHKSGQN | 1800 | GSGSPHSKGQN | 1910 | GSGSPHASR | 2020 | GHDSPHKSA | 2130 |
| QQ | | QQ | | QNKQ | | QNYQ | |
| GSGSPHSKAQN | 1801 | DGGSPHSKAQN | 1911 | GSGSPHVKIQ | 2021 | GSASPHSKA | 2131 |
| QQ | | QQ | | NQQ | | LNQQ | |
| GSGSPHSKAQN | 1802 | ASGSPHSKAHN | 1912 | GSGSPHSKA | 2022 | GHESPHKSA | 2132 |
| RH | | QQ | | KNQQ | | QNRQ | |
| GSGSPHSKVQN | 1803 | GSGSPHSKAQN | 1913 | GSGSPHKKN | 2023 | GQDSPHKIG | 2133 |
| QQ | | TY | | QNQQ | | QNQQ | |
| MSGSPHSKAQN | 180 | GSGSPHSKSQN | 1914 | GSGSPHVRM | 2024 | GHDSPHKSG | 2134 |
| QQ | | QH | | QNQQ | | QNHL | |
| GRGSPHSKAQN | 1805 | GGGSPHSKAQD | 1915 | GSGSPHASR | 2025 | GHDSPHKSG | 2135 |
| QQ | | KQ | | QKQQ | | QYQH | |
| RNGSPHSKAQN | 1806 | GSGSPHSKAQN | 1916 | GHSSPHRSG | 2026 | GNDSPHKSV | 2136 |
| QQ | | HL | | QNQQ | | QNHQ | |
| GSGSPHSKARD | 1807 | GSGSPHSKAQIG | 1917 | GSGSPHTRG | 2027 | GHDSPHKSG | 2137 |
| QQ | | M | | QNQQ | | QNQW | |
| GSGSPHSKAPNL | 1808 | GSGSPHSKALN | 1918 | CSGSPHSKA | 2028 | GHDSPHKSV | 2138 |
| Q | | KQ | | QNQQ | | QNQH | |
| TSGSPHSKAQN | 1809 | GGGSPHSKAQN | 1919 | GSGSPHRKA | 2029 | GHDSPHKSG | 2139 |
| QQ | | PQ | | QNQQ | | QNQH | |
| GSGSPHSKAHV | 1810 | GTGSPHSKAPN | 1920 | GSGSPHSKA | 2030 | GHDSPHKSG | 2140 |
| RQ | | QL | | QNKQ | | QTRQ | |
| GSGSPHSKAPNQ | 1811 | GSGSPHSKAQL | 1921 | GSGSPHSKA | 2031 | GHDSPHKSG | 2141 |
| H | | QQ | | QTQQ | | QNLH | |
| ISGSPHSKAQNQ | 1812 | GGGSPHSKAQY | 1922 | CSGSPHKTSQ | 2032 | GHDSPHKSA | 2142 |
| Q | | QQ | | NQQ | | QNQE | |
| GPGSPHSKAHN | 1813 | GGGSPHSKAQH | 1923 | CSHSPHKSG | 2033 | GHDSPHKSG | 2143 |
| QQ | | QQ | | QNQQ | | QHLQ | |
| GSGSPHSKTQSQ | 1814 | GSGSPHSKAQR | 1924 | GQSSPHRSG | 2034 | GHDSPHKSR | 2144 |
| Q | | MS | | QNQQ | | LNQP | |
| ESGSPHSKAQN | 1815 | GSGSPHSKAQGI | 1925 | GRGSPHASR | 2035 | GQDSPHKSG | 2145 |
| QQ | | L | | QNQQ | | QNQD | |
| GSGSPHSKAQPA | 1816 | GSGSPHSKAQD | 1926 | GSGSPHASR | 2036 | GHDSPHKSG | 2146 |
| K | | RQ | | KNQQ | | RNQQ | |
| SSGSPHSKAQNQ | 1817 | GSGSPHSKARD | 1927 | GSGSPHASR | 2037 | GHDSPHKSG | 2147 |
| Q | | WQ | | QNQH | | QNLL | |
| GNGSPHSKAQN | 1818 | GSGSPHSKAQN | 1928 | GSGSPHKFG | 2038 | GHDSPHKSG | 2148 |
| QQ | | TH | | KNQQ | | QLVI | |
| GSGSPHSKSQTQ | 1819 | GSGSPHSKAQE | 1929 | GSGSPHKFG | 2039 | GHDSPHKSR | 2149 |
| Q | | RS | | QKQQ | | QSQQ | |
| ASGSPHSKAQN | 1820 | GSGSPHSKAQN | 1930 | GSGSPHKFG | 2040 | GHDSPHKSG | 2150 |
| QQ | | YQ | | QNQQ | | RTQE | |
| GSGSPHSKAQN | 1821 | GSGSPHSKAQR | 1931 | GSGSPHKIGQ | 2041 | GHDSPHKSV | 2151 |
| LA | | TC | | NQQ | | QTHQ | |
| GSGSPHSKSQNQ | 1822 | GSGSPHSKAQIG | 1932 | GSGSPHKLG | 2042 | GHDSPHKSG | 2152 |
| L | | H | | QNQQ | | QNQP | |
| NSGSPHSKAQN | 1823 | GSGSPHSKAQG | 1933 | GSGSPHKTSK | 2043 | GHDSPHKSG | 2153 |
| QQ | | AI | | NQQ | | QTQQ | |
| GSGSPHSKAQG | 1824 | GSGSPHSKAQV | 1934 | GSGSPHKTSQ | 2044 | GPDSPHKIGQ | 2154 |
| QQ | | PP | | KQQ | | NQQ | |
| VSGSPHSKAQN | 1825 | GSGSPHSKAQV | 1935 | GSGSPHKTSQ | 2045 | GHDSPHKSV | 2155 |
| QQ | | QQ | | NQQ | | QNQQ | |
| GSGSPHSKALNR | 1826 | GSGSPHSKALM | 1936 | GSGSPHKTT | 2046 | GHDSPHKSR | 2156 |
| Q | | RQ | | QNQQ | | QDQH | |
| LSGSPHSKAQN | 1827 | GSGSPHSKAQY | 1937 | GSGSPHKTY | 2047 | GPDSPHKSG | 2157 |
| QQ | | SV | | QNQQ | | QKQQ | |
| GSGSPHSKAHN | 1828 | GSGSPHSKVPNL | 1938 | GSGSPHKYG | 2048 | GHDSPHKSR | 2158 |
| QQ | | Q | | QNQQ | | QSQH | |
| GSGSPHSKTQN | 1829 | GSGSPHSKAQAI | 1939 | GSGSPHSKA | 2049 | GHDSPHKSV | 2159 |
| QQ | | T | | QHQQ | | QNQL | |
| GGGSPHSKAQT | 1830 | GSGSPHSKAQK | 1940 | GSGSPHSKD | 2050 | GYDSPHKSG | 2160 |
| QQ | | TL | | QNQQ | | QYQH | |
| GSGSPHSKAQNP | 1831 | GSGSPHSKAQN | 1941 | GSGSPHSKPQ | 2051 | GHDSPHKSR | 2161 |
| P | | QW | | NQQ | | QNQQ | |
| GSGSPHSKAQN | 1832 | GSGSPHSKAQL | 1942 | GSGSPHVRG | 2052 | GHDSPHKSW | 2162 |
| LQ | | HH | | QKQQ | | VRQQ | |
| GGGSPHSKAQN | 1833 | GSGSPHSKAQNI | 1943 | GSGSPHVRG | 2053 | GHESPHKSG | 2163 |
| QQ | | I | | QNKQ | | QNQH | |
| GSGSPHSKAQY | 1834 | MEGSPHSKAQN | 1944 | GSHSPHKRG | 2054 | GHDSPHKIG | 2164 |
| QQ | | QQ | | QNQQ | | HNQQ | |
| GGGSPHSKAQN | 1835 | GSGSPHSKAQG | 1945 | GSHSPHKSG | 2055 | GHDSPHKSN | 2165 |
| KQ | | HH | | QKQQ | | AWQQ | |
| GSGSPHSKAQD | 1836 | GSGSPHSKAQS | 1946 | GSHSPHKSG | 2056 | GHDSPHKSG | 2166 |
| QE | | KV | | QNQQ | | QSVP | |
| KSGSPHSKAQN | 1837 | GSGSPHSKAQLP | 1947 | VSGSPHASR | 2057 | GHESPHKSG | 2167 |
| QQ | | S | | QNQQ | | QNIQ | |
| GGGSPHSKAQN | 1838 | GSGSPHSKAIGK | 1948 | VSGSPHGAR | 2058 | GHDSPHKSV | 2168 |
| QL | | Q | | QNQQ | | QNHL | |
| GSGSPHSKAQN | 1839 | GGGSPHSKSQN | 1949 | VSGSPHKFG | 2059 | GHDSPHKIG | 2169 |
| HQ | | QQ | | QNQQ | | LDQQ | |
| GSGSPHSKAQD | 1840 | GSGSPHSKAQAI | 1950 | GSGSPHSKA | 2060 | ASGSPHSKA | 2170 |
| QQ | | H | | QYYV | | QHQQ | |
| GGGSPHSKSQN | 1841 | GSGSPHSKAQH | 1951 | GSGSPHSKLR | 2061 | GHDSPHKRG | 2171 |
| QL | | GL | | RQQ | | PDQQ | |
| GNGSPHSKAQN | 1842 | GSGSPHSKAQF | 1952 | GSGSPHSKA | 2062 | GMGSPHSKT | 2172 |
| KQ | | MC | | GCGQ | | QNQQ | |
| GSGSPHSKGHW | 1843 | VSGSPHSKAQG | 1953 | GSGSPHSRA | 2063 | GHDSPHKSG | 2173 |
| QQ | | QQ | | QNQQ | | ESQQ | |
| GSGSPHSKAPNQ | 1844 | GGGSPHSKAQN | 1954 | GSGSPHSKRL | 2064 | GHDSPHKHG | 2174 |
| Q | | QM | | RQQ | | QNHQ | |
| GSGSPHSKAQN | 1845 | GSGSPHSKAQH | 1955 | GSGSPHSLRR | 2065 | GTGSPHSKA | 2175 |
| QL | | LQ | | NQQ | | QNQL | |
| GSGSPHSKRPEQ | 1846 | ENGSPHSKAQN | 1956 | GSGSPHSRGR | 2066 | GHDSPHKSV | 2176 |
| Q | | QQ | | NQQ | | QNKQ | |
| GSGSPHSKAQRT | 1847 | GSGSPHSKTQN | 1957 | GSGSPHSSRR | 2067 | GQVSPHKSG | 2177 |
| M | | HQ | | NQQ | | QNQQ | |
| GNGSPHSKAQN | 1848 | GSGSPHSKAQP | 1958 | GSGSPHSKAF | 2068 | GHDSPHKSG | 2178 |
| QH | | AR | | RLQ | | QRQL | |
| HSGSPHSKAQN | 1849 | GSGSPHSKAQSL | 1959 | GSCSPHRKA | 2069 | GHDSPHKIG | 2179 |
| QQ | | Q | | QNQQ | | QNQL | |
| GGGSPHSKALN | 1850 | GSGSPHSKSQSQ | 1960 | GSGSPHFLRQ | 2070 | GHDSPHKSG | 2180 |
| QQ | | L | | NQQ | | QIIV | |
| GSGSPHSKALH | 1851 | GSASPHSKAHS | 1961 | GSGSPHSLRF | 2071 | GYDSPHKSG | 2181 |
| QH | | QQ | | NQQ | | QKQS | |
| GTGSPHSKAQN | 1852 | GSGSPHSKAQM | 1962 | GSGSPHSKW | 2072 | GNGSPHSKA | 2182 |
| HQ | | PS | | LLQQ | | QNQE | |
| GSGSPHSKAQH | 1853 | GSGSPHSKAQG | 1963 | GSGSPHSKRR | 2073 | GDDSPHKSV | 2183 |
| RI | | SL | | LQQ | | QNQQ | |
| GSGSPHSKAQYI | 1854 | GSGSPHSKSQN | 1964 | GSGSPHSKA | 2074 | GHDSPHKSV | 2184 |
| H | | QQ | | QRKL | | QSHQ | |
| GGGSPHSKAHN | 1855 | GNGSPHSKSQN | 1965 | GSGSPHSKAL | 2075 | GHDSPHKSG | 2185 |
| QQ | | QQ | | RRQ | | QFVV | |
| GSGSPHSKAQKF | 1856 | GSGSPHSKAQV | 1966 | GSGSPHSKA | 2076 | GHDSPHKSR | 2186 |
| E | | PA | | QRLR | | QNLQ | |
| ESGSPHSKAQN | 1857 | GNGSPHSKAQN | 1967 | GSGSPHSKA | 2077 | GHNSPHKSG | 2187 |
| HQ | | LQ | | QRRL | | QNQE | |
| GSGSPHSKAQFP | 1858 | GSGSPHSKAQD | 1968 | GSGSPHSKA | 2078 | GHDSPHKSG | 2188 |
| S | | KQ | | RRQQ | | QSQP | |
| PSGSPHSKAQNQ | 1859 | GSGSPHSKAHY | 1969 | GSGSPHSKA | 2079 | GHESPHKSG | 2189 |
| Q | | QQ | | RRLQ | | QNEQ | |
| GNGSPHSKAQN | 1860 | GSGSPHSKAQV | 1970 | GSGSPHSKSR | 2080 | GHDSPHKSG | 2190 |
| PL | | PS | | RQQ | | QNQL | |
| GGGSPHSKAQS | 1861 | GGGSPHSKAQN | 1971 | GLLSPHWKA | 2081 | GHDSPHKSA | 2191 |
| QQ | | HQ | | QNQQ | | QNLL | |
| GSGSPHSKAQAI | 1862 | GSGSPHSKARG | 1972 | GSGSPHSKA | 2082 | ASGSPHSKAI | 2192 |
| K | | EQ | | RLRQ | | NQQ | |
| GSGSPHSKGQN | 1863 | GGGSPHSKAQY | 1973 | GSGSPHSKAS | 2083 | GNGSPHKRG | 2193 |
| RQ | | QH | | KRQ | | QNQQ | |
| GSGSPHSKAQSQ | 1864 | GSGSPHSKAPG | 1974 | GSGSPHVRR | 2084 | GHDSPHKSG | 2194 |
| Q | | QQ | | QNQQ | | QSLQ | |
| GSVSPHGKAQN | 1865 | KNGSPHSKAQN | 1975 | GSGSPHSKA | 2085 | GHDSPHKSA | 2195 |
| QL | | QQ | | QLYR | | QNHQ | |
| ASGSPHSKAQN | 1866 | GSGSPHSKRLEQ | 1976 | GSGSPHSKA | 2086 | GHDSPHKSG | 2196 |
| QL | | Q | | QLTV | | RNRQ | |
| RSGSPHSKAQN | 1867 | GSGSPHSKAQN | 1977 | GHDSPHKRG | 2087 | GHDSPHKYG | 2197 |
| QQ | | QS | | QHRQ | | QNEQ | |
| GSGSPHSKAQY | 1868 | GSGSPHSKAQK | 1978 | GHDSPHKSG | 2088 | GNGSPHSKA | 2198 |
| QH | | VI | | QKQQ | | PNLQ | |
| GSGSPHTKAQN | 1869 | GSGSPHSKAQN | 1979 | GHDSPHKSG | 2089 | GHDSPHKSQ | 2199 |
| PQ | | ND | | LTQQ | | QNQQ | |
| GSGSPHSKGQNP | 1870 | GSGSPHSKAQS | 1980 | GDDSPHKSG | 2090 | GHDSPHKSV | 2200 |
| P | | VH | | RNQQ | | QSKQ | |
| GSGSPHSKAQH | 1871 | GSGSPHSKAQPL | 1981 | GHDSPHKSG | 2091 | GNDSPHKIG | 2201 |
| QL | | G | | LNQQ | | HNQQ | |
| GSGSPHSKAQSP | 1872 | KEGSPHSKAQN | 1982 | GHESPHKSA | 2092 | GGGSPHSKA | 2202 |
| P | | QQ | | QNHQ | | QDQQ | |
| GSGSPHSKAQA | 1873 | GSGSPHSKAHN | 1983 | GHDSPHKSA | 2093 | GQDSPHKSG | 2203 |
| KL | | QE | | QNQW | | QNPL | |
| GSGSPHSKTKSQ | 1874 | GSGSPHSKAQIQ | 1984 | GHDSPHKSG | 2094 | ASGSPHSKA | 2204 |
| Q | | Q | | QNTH | | QNHQ | |
| GSGSPHSKAQD | 1875 | GSGSPHSKAQV | 1985 | GHDSPHKSG | 2095 | GHDSPHKSG | 2205 |
| RP | | RN | | RRRQ | | RDQK | |
| GIGSPHSKAQNL | 1876 | GSGSPHSKAPSN | 1986 | GHDSPHKSA | 2096 | GHDSPHKSV | 2206 |
| G | | Q | | QNQQ | | HNQQ | |
| GSGSPHSKAQAF | 1877 | GSGSPHSKAQV | 1987 | GHDSPHKSG | 2097 | GHDSPHKSG | 2207 |
| H | | GH | | QYQQ | | QWKR | |
| GSGSPHSKAQK | 1878 | GSGSPHSKAQR | 1988 | GNYSPHKIG | 2098 | GSGSPHSKA | 2208 |
| QQ | | DI | | QNQQ | | ENRQ | |
| GSGSPHSKAQN | 1879 | GSGSPHSKAQM | 1989 | GHDSPHKSR | 2099 | GHDSPHKSG | 2209 |
| AQ | | PN | | QNDQ | | QSQQ | |
| WSGSPHSKAQN | 1880 | AIGSPHSKAQNQ | 1990 | GHDSPHKSG | 2100 | GHDSPHKSR | 2210 |
| QQ | | Q | | QIRQ | | QAQQ | |
| GSGSPHSKAHN | 1881 | GSGSPHSKARG | 1991 | GHDSPHKIG | 2101 | GHDSPHKSV | 2211 |
| QL | | LQ | | QNQH | | QNHQ | |
| GNGSPHSKAQN | 1882 | GSGSPHSKLQK | 1992 | GYDSPHKSG | 2102 | GHDSPHKSK | 2212 |
| HQ | | QQ | | QKQQ | | QNQQ | |
| GGGSPHSKAQN | 1883 | GSGSPHSKAPSL | 1993 | GHDSPHKSG | 2103 | GHDSPHKSA | 2213 |
| LQ | | Q | | QSVQ | | QNQL | |
| GSGSPHSKAQK | 1884 | GSGSPHSKAQR | 1994 | GHESPHKSG | 2104 | GHDSPHKSG | 2214 |
| LN | | DQ | | RSQQ | | QTQP | |
| GGGSPHSKSQN | 1885 | GSGSPHSKNRD | 1995 | GHDSPHKSG | 2105 | GHDSPHKLW | 2215 |
| QH | | QQ | | QNKL | | INQQ | |
| GSGSPHSKSQNV | 1886 | GSGSPHSKAQA | 1996 | GHDSPHKTG | 2106 | GPDSPHKSG | 2216 |
| Q | | KG | | QNQQ | | QNQQ | |
| GSGSPHSKAQA | 1887 | GSGSPHSKAQS | 1997 | GRGSPHKRG | 2107 | GHDSPHKSV | 2217 |
| QQ | | AH | | QNQQ | | QKQL | |
| DSGSPHSKAQN | 1888 | GNGSPHSKSQN | 1998 | GSGSPHTKA | 2108 | GHPSPHWKG | 2218 |
| QQ | | QH | | QNPP | | QNQQ | |
| ASGSPHSKAPNQ | 1889 | GSGSPHSKSQN | 1999 | GQDSPHKSG | 2109 | GHDSPHKSG | 2219 |
| Q | | HQ | | QHQQ | | RNQL | |
| GSGSPHSKAQTP | 1890 | RSGSPHSKAQD | 2000 | GHDSPHKSG | 2110 | GSGSPHSKV | 2220 |
| P | | QQ | | QIQH | | QDQQ | |
| IDGSPHSKAQNQ | 1891 | GSGSPHSKAQST | 2001 | GHDSPHKSG | 2111 | GHDSPHKMG | 2221 |
| Q | | M | | PRQQ | | RNQQ | |
| GSGSPHNKAQN | 1892 | GSGSPHSKAQR | 2002 | GHDSPHKSG | 2112 | GHDSPHKSGI | 2222 |
| HQ | | EM | | HTQQ | | SIQ | |
| GSGSPHSKAQPP | 1893 | GGGSPHSKSQN | 2003 | GHDSPHKSG | 2113 | GHDSPHKSV | 2223 |
| A | | RQ | | QRQH | | QNLQ | |
| GSGSPHSKAQER | 1894 | GSGSPHSKAQY | 2004 | GSGSPHTKA | 2114 | GHDSPHKMA | 2224 |
| P | | RA | | QNQQ | | HNQQ | |
| GSGSPHSKAQD | 1895 | GGGSPHSKAQR | 2005 | GHDSPHKSA | 2115 | GHDSPHKHG | 2225 |
| LQ | | QQ | | QSQQ | | QNQQ | |
| GGGSPHSKAQN | 1896 | GSGSPHSKNQW | 2006 | GHESPHKSG | 2116 | GHDSPHKSV | 2226 |
| PP | | QQ | | QNQQ | | QSQQ | |
| GSGSPHSKAQA | 1897 | GSGSPHSKAQR | 2007 | GHDSPHKSL | 2117 | GHDSPHKSG | 2227 |
| MH | | MN | | QNQQ | | QTVC | |
| GSGSPHSKALN | 1898 | GSGSPHAKAQN | 2008 | GHGSPHSKA | 2118 | GQDSPHKSG | 2228 |
| QQ | | HQ | | QNPQ | | QYQQ | |
| GSGSPHSKAQHP | 1899 | GSGSPHSKAGD | 2009 | GHDSPHKSG | 2119 | GHDSPHKSG | 2229 |
| S | | SQ | | RNQE | | QQIM | |
| GLGSPHSKSQN | 1900 | GSGSPHSKLKSQ | 2010 | GHDSPHKSG | 2120 | GHDSPHKSR | 2230 |
| QQ | | Q | | QTQL | | QNEQ | |
| GTGSPHSKAQN | 1901 | GSGSPHSKAQKI | 2011 | GHDSPHKSE | 2121 | GHDSPHKSG | 2231 |
| QQ | | S | | KNQQ | | LNHQ | |
| GSGSPHSKAPGL | 1902 | GSGSPHSKAPS | 2012 | GRDSPHKSG | 2122 | GYDSPHKSG | 2232 |
| Q | | MQ | | QDQQ | | QNQQ | |
| GSGSPHSKAQGI | 1903 | GSGSPHSKASPR | 2013 | GHDSPHKTG | 2123 | GHDSPHKSG | 2233 |
| R | | 0 | | HNQQ | | QNLQ | |
| GSGSPHSKAQAP | 1904 | GSGSPHSKRME | 2014 | GYDSPHKSG | 2124 | GHDSPHKSR | 2234 |
| A | | QQ | | QTQQ | | QDQQ | |
| GSGSPHSKSQSQ | 1905 | GSGSPHSKAQY | 2015 | GHESPHKSG | 2125 | GDDSPHKSG | 2235 |
| Q | | QN | | QTQQ | | QKQL | |
| GSGSPHSKAQIP | 1906 | GSGSPHARMQN | 2016 | GHDSPHKSG | 2126 | GSGSPHSKA | 2236 |
| P | | QQ | | QSKQ | | QNQA | |
| GSGSPHSKAQT | 1907 | GSGSPHVKSQN | 2017 | GHDSPHKTG | 2127 | GDDSPHKSG | 2237 |
| QL | | QQ | | QNQP | | HNQQ | |
| GSGSPHSKAQAP | 1908 | GQDSPHKSGQN | 2018 | GHDSPHKSG | 2128 | GHDSPHKSG | 2238 |
| S | | QQ | | QSPQ | | QMIH | |
| GHDSPHKSGRN | 2239 | GHDSPHKSVQN | 2240 | GHDSPHKSG | 2241 | TINGHDSPH | 2728 |
| HQ | | RQ | | QKMN | | KSRLNQP | |
| TINGSGSPHSKA | 2242 | TDRGSGSPHSK | 2404 | TINGSGSPHS | 1314 | TVDGHDSPH | 2729 |
| QNQQ | | AQNQQ | | KAQSTM | | KSGQKQQ | |
| TINGHDSPHKSG | 2243 | TINGSGSPHSKA | 2405 | TVNASGSPHS | 1315 | TINGQDSPH | 2730 |
| QNQQ | | QIPP | | KAQNQL | | KSGQNQD | |
| TIIGSGSPHSKAQ | 2244 | TVKGSGSPHSK | 2406 | TINGSGSPHS | 1316 | TIEGHDSPHK | 2731 |
| NRH | | AQDQQ | | KAQREM | | SGRNQQ | |
| TFPGSGSPHSKV | 2245 | NADGSGSPHSK | 2407 | TVHGSGSPHS | 1317 | TTNGHDSPH | 2732 |
| QNQQ | | AQNQQ | | KAQSQQ | | KSGQNLL | |
| TEKMSGSPHSK | 2246 | TDKVSGSPHSK | 2408 | TINGGGSPHS | 1318 | TINGHDSPH | 2733 |
| AQNQQ | | AQNQQ | | KSQNRQ | | KSGQLVI | |
| EINGRGSPHSKA | 2247 | TITGSGSPHSKA | 2409 | TINGSGSPHS | 1319 | TVNGHDSPH | 2734 |
| QNQQ | | QTQL | | KAQYRA | | KSRQSQQ | |
| TVNRNGSPHSK | 2248 | TINGSGSPHSKA | 2410 | TINGGGSPHS | 1320 | TINGHDSPH | 2735 |
| AQNQQ | | QAPS | | KAQRQQ | | KSGRTQE | |
| TVNGSGSPHSK | 2249 | NCVGSGSPHSK | 2411 | TEPMSGSPHS | 1321 | TINGHDSPH | 2736 |
| ARDQQ | | AQNQQ | | KAQNQQ | | KSVQTHQ | |
| TFNGSGSPHSKA | 2250 | TIRDAGSPHSKA | 2412 | TINGSGSPHS | 1322 | TSNGHDSPH | 2737 |
| PNLQ | | QNQQ | | KNQWQQ | | KSGQNQP | |
| TEKTSGSPHSKA | 2251 | TVKDSGSPHSK | 2413 | ETAGSGSPHS | 1323 | VINGHDSPH | 2738 |
| QNQQ | | AQNQQ | | KAQNQQ | | KSGQTQQ | |
| TINGSGSPHSKA | 2252 | NALGSGSPHSK | 2414 | TINGSGSPHS | 1324 | TINGPDSPHK | 2739 |
| HVRQ | | AQNQQ | | KAQRMN | | IGQNQQ | |
| TVNGSGSPHSK | 2253 | VINGSGSPHSKG | 2415 | NNLGSGSPHS | 1325 | AVNGHDSPH | 2740 |
| APNQH | | QNQQ | | KAQNQQ | | KSVQNQQ | |
| TEKISGSPHSKA | 2254 | TVNGGGSPHSK | 2416 | TINGSGSPHA | 1326 | TINGHDSPH | 2741 |
| QNQQ | | AQNQQ | | KAQNHQ | | KSRQDQH | |
| TINGPGSPHSKA | 2255 | TIQDGGSPHSKA | 2417 | TIIKNGSPHS | 1327 | AINGPDSPHK | 2742 |
| HNQQ | | QNQQ | | KAQNQQ | | SGQKQQ | |
| TVNGSGSPHSKT | 2256 | TISGGGSPHSKA | 2418 | TINGSGSPHS | 2580 | TINGHDSPH | 2743 |
| QSQQ | | QNQQ | | KAGDSQ | | KSRQSQH | |
| SINESGSPHSKA | 2257 | TSNASGSPHSKA | 2419 | TINGSGSPHS | 2581 | TIYGHDSPH | 2744 |
| QNQQ | | HNQQ | | KLKSQQ | | KSVQNQL | |
| TERTSGSPHSKA | 2258 | TINGSGSPHSKA | 2420 | TINGSGSPHS | 2582 | TVNGHDSPH | 2745 |
| QNQQ | | QNTY | | KAQKIS | | KSGQNLL | |
| TINGSGSPHSKA | 2259 | TINGSGSPHSKS | 2421 | TEYNSGSPHS | 2583 | TENKSGSPHS | 2746 |
| QPAK | | QNQH | | KAQNQQ | | KAQNQQ | |
| TEKSSGSPHSKA | 2260 | TINGGGSPHSKA | 2422 | TINGSGSPHS | 2584 | TTNGQDSPH | 2747 |
| QNQQ | | QDKQ | | KAPSMQ | | KSGQNQQ | |
| TSYGNGSPHSK | 2261 | TEFVSGSPHSKA | 2423 | AINGSGSPHS | 2585 | TDKGSGSPH | 2748 |
| AQNQQ | | QNQQ | | KAQNQQ | | SKAQNQQ | |
| TEKGSGSPHSKA | 2262 | TVNGSGSPHSK | 2424 | TINGSGSPHS | 2586 | TIDGHDSPH | 2749 |
| QNQQ | | AQNHL | | KASPRQ | | KSGRNQQ | |
| TINGSGSPHSKS | 2263 | TREISGSPHSKA | 2425 | TINGSGSPHS | 2587 | TINGYDSPH | 2750 |
| QTQQ | | QNQQ | | KRMEQQ | | KSGQYQH | |
| TERISGSPHSKA | 2264 | TINGSGSPHSKA | 2426 | TINGSGSPHS | 2588 | TDNGHDSPH | 2751 |
| QNQQ | | QIGM | | KAQYQN | | KSRQNQQ | |
| TERASGSPHSKA | 2265 | TIDGSGSPHSKA | 2427 | TINGSGSPHS | 2589 | TINGHDSPH | 2752 |
| QNQQ | | LNKQ | | KAQYYV | | KSWVRQQ | |
| ELHGSGSPHSKA | 2266 | TIIGGGSPHSKA | 2428 | TINGSGSPHS | 2590 | TINGHESPHK | 2753 |
| QNQQ | | QNPQ | | KLRRQQ | | SGQNQH | |
| AINGSGSPHSKA | 2267 | QGEGSGSPHSK | 2429 | TINGSGSPHS | 2591 | TVNGHDSPH | 2754 |
| QNLA | | AQNQQ | | KAGCGQ | | KIGHNQQ | |
| TVNGSGSPHSKS | 2268 | TINGTGSPHSKA | 2430 | SMNGSGSPH | 2592 | TCNGHDSPH | 2755 |
| QNQL | | PNQL | | SRAQNQQ | | KSGRNQQ | |
| TERNSGSPHSKA | 2269 | TVNGSGSPHSK | 2431 | TINGSGSPHS | 2593 | TINGNGSPHS | 2756 |
| QNQQ | | AQLQQ | | KRLRQQ | | KAQNHQ | |
| SVNGNGSPHSK | 2270 | TFNGGGSPHSK | 2432 | TINGSGSPHS | 2594 | NVVGHDSPH | 2757 |
| AQNQQ | | AQYQQ | | LRRNQQ | | KSGQNQQ | |
| TFNGSGSPHSKA | 2271 | SINGSGSPHSKT | 2433 | TINGSGSPHS | 2595 | TINGHDSPH | 2758 |
| QGQQ | | QSQQ | | RGRNQQ | | KSNAWQQ | |
| TERVSGSPHSKA | 2272 | TVNGGGSPHSK | 2434 | TINGSGSPHS | 2596 | TDAGHDSPH | 2759 |
| QNQQ | | AQHQQ | | SRRNQQ | | KSGQNQQ | |
| TINGSGSPHSKA | 2273 | SEKGSGSPHSKA | 2435 | TINGSGSPHS | 2597 | TEVGHDSPH | 2760 |
| LNRQ | | QNQQ | | KAFRLQ | | KSGQNQQ | |
| TERLSGSPHSKA | 2274 | NVNGSGSPHSK | 2436 | TINGSCSPHR | 2598 | SELGHDSPH | 2761 |
| QNQQ | | AQNQQ | | KAQNQQ | | KSGQNQQ | |
| TDNGSGSPHSK | 2275 | GGEGSGSPHSK | 2437 | TINGSGSPHF | 2599 | TINGHDSPH | 2762 |
| AHNQQ | | AQNQQ | | LRQNQQ | | KSGQSVP | |
| TFHGSGSPHSKT | 2276 | TINGSGSPHSKA | 2438 | TINGSGSPHS | 2600 | TINGHESPHK | 2763 |
| QNQQ | | QRMS | | LRFNQQ | | SGQNIQ | |
| TINGGGSPHSKA | 2277 | TINGSGSPHSKA | 2439 | TINGSGSPHS | 2601 | TINGHDSPH | 2764 |
| QTQQ | | QGIL | | KWLLQQ | | KSVQNHL | |
| TSNGSGSPHSKA | 2278 | EFVGSGSPHSKA | 2440 | TINGSGSPHS | 2602 | TINGHDSPH | 2765 |
| QNPP | | QNQQ | | KRRLQQ | | KIGLDQQ | |
| TINGSGSPHSKA | 2279 | TIIGSGSPHSKAQ | 2441 | TINGSGSPHS | 2603 | TSNASGSPHS | 2766 |
| QNLQ | | DRQ | | KAQRKL | | KAQHQQ | |
| TVHGNGSPHSK | 2280 | SDKGSGSPHSK | 2442 | TINGSGSPHS | 2604 | TINGHDSPH | 2767 |
| AQNQQ | | AQNQQ | | KALRRQ | | KRGPDQQ | |
| TINGGGSPHSKA | 2281 | TEQVSGSPHSKA | 2443 | TINGSGSPHS | 2605 | TINGMGSPH | 2768 |
| QNQQ | | QNQQ | | KAQRLR | | SKTQNQQ | |
| TENMSGSPHSK | 2282 | TEHVSGSPHSKA | 2444 | YLSGSGSPHS | 2606 | TIKGHDSPH | 2769 |
| AQNQQ | | QNQQ | | KAQNQQ | | KSGESQQ | |
| TENVSGSPHSKA | 2283 | TINGSGSPHSKA | 2445 | TINGSGSPHS | 2607 | TINGHDSPH | 2770 |
| QNQQ | | RDWQ | | KAQRRL | | KHGQNHQ | |
| TSSGSGSPHSKA | 2284 | TENASGSPHSKA | 2446 | TINGSGSPHS | 2608 | TVNGTGSPH | 2771 |
| QYQQ | | QNQQ | | KARRQQ | | SKAQNQL | |
| TIDGGGSPHSKA | 2285 | EVQGSGSPHSK | 2447 | TINGSGSPHS | 2609 | TIIGHDSPHK | 2772 |
| QNKQ | | AQNQQ | | KARRLQ | | SGQYQH | |
| TEKVSGSPHSKA | 2286 | TINGSGSPHSKA | 2448 | TINGSGSPHS | 2610 | TSNGHDSPH | 2773 |
| QNQQ | | QNTH | | KSRRQQ | | KSVQNKQ | |
| AINGSGSPHSKA | 2287 | TINGSGSPHSKA | 2449 | TINGLLSPHW | 2611 | IVNGQVSPH | 2774 |
| QDQE | | PNLQ | | KAQNQQ | | KSGQNQQ | |
| TCNKSGSPHSKA | 2288 | TINGSGSPHSKA | 2450 | TINGSGSPHS | 2612 | TVNGHDSPH | 2775 |
| QNQQ | | QERS | | KARLRQ | | KSGQRQL | |
| TINGGGSPHSKA | 2289 | TSNGSGSPHSKA | 2451 | TINGSGSPHS | 2613 | TVNGHDSPH | 2776 |
| QNQL | | QNYQ | | KASKRQ | | KIGQNQL | |
| NINGGGSPHSKA | 2290 | TEYISGSPHSKA | 2452 | TINGSGSPHV | 2614 | TINGHDSPH | 2777 |
| QNQQ | | QNQQ | | RRQNQQ | | KSGQIIV | |
| TEHLSGSPHSKA | 2291 | TINGSGSPHSKA | 2453 | TINGSGSPHS | 2615 | IGNGHESPH | 2778 |
| QNQQ | | QRTC | | KAQLYR | | KSGQNQQ | |
| AEMGSGSPHSK | 2292 | TINGSGSPHSKA | 2454 | GLSGSGSPHS | 2616 | EVMGHDSPH | 2779 |
| AQNQQ | | QIGH | | KAQNQQ | | KSGQNQQ | |
| ATNGSGSPHSK | 2293 | NCWGSGSPHSK | 2455 | TINGSGSPHS | 2617 | TINGYDSPH | 2780 |
| AQNHQ | | AQNQQ | | KAQLTV | | KSGQKQS | |
| AIKGSGSPHSKA | 2294 | TINGSGSPHSKA | 2456 | TINGHDSPHK | 2618 | TIHGNGSPHS | 2781 |
| QDQQ | | QGAI | | RGQHRQ | | KAQNQE | |
| TINGGGSPHSKS | 2295 | TDVNSGSPHSK | 2457 | MPEGHDSPH | 2619 | YQVGHDSPH | 2782 |
| QNQL | | AQNQQ | | KSGQNQQ | | KSGQNQQ | |
| TVNGNGSPHSK | 2296 | SDIGSGSPHSKA | 2458 | MEGGHDSPH | 2620 | TIKGDDSPH | 2783 |
| AQNKQ | | QNQQ | | KSGQNQQ | | KSVQNQQ | |
| TINGSGSPHSKG | 2297 | TINGSGSPHSKA | 2459 | MEYGHDSPH | 2621 | TINGHDSPH | 2784 |
| HWQQ | | QVPP | | KSGQNQQ | | KSVQSHQ | |
| TDKTSGSPHSKA | 2298 | TINGSGSPHSKA | 2460 | AEWGHDSPH | 2622 | TINGHDSPH | 2785 |
| QNQQ | | QVQQ | | KSGQNQQ | | KSGQFVV | |
| TFKGSGSPHSKA | 2299 | TINGSGSPHSKA | 2461 | CEWGHDSPH | 2623 | TVNGHDSPH | 2786 |
| PNQQ | | LMRQ | | KSGQNQQ | | KSRQNLQ | |
| TVNGSGSPHSK | 2300 | TINGSGSPHSKA | 2462 | ANNGQDSPH | 2624 | ATNGHNSPH | 2787 |
| AQNQL | | QYSV | | KSGQNQQ | | KSGQNQE | |
| TINGSGSPHSKR | 2301 | NSIGSGSPHSKA | 2463 | IPEGHDSPHK | 2625 | AINGHDSPH | 2788 |
| PEQQ | | QNQQ | | SGQNQQ | | KSAQNQQ | |
| TINGSGSPHSKA | 2302 | TINGSGSPHSKV | 2464 | ADMGHDSPH | 2626 | TEHGHDSPH | 2789 |
| QRTM | | PNLQ | | KSGQNQQ | | KSGQNQQ | |
| TEKASGSPHSKA | 2303 | AINGSGSPHSKA | 2465 | IEYGHDSPHK | 2627 | TIYGHDSPH | 2790 |
| QNQQ | | QSQQ | | SGQNQQ | | KSGQSQP | |
| SDQGSGSPHSKA | 2304 | TINGSGSPHSKA | 2466 | ADYGHDSPH | 2628 | TISGHESPHK | 2791 |
| QNQQ | | QAIT | | KSGQNQQ | | SGQNEQ | |
| TEITSGSPHSKA | 2305 | TINGSGSPHSKA | 2467 | IETGHDSPHK | 2629 | AIIGHDSPHK | 2792 |
| QNQQ | | QKTL | | SGQNQQ | | SAQNQQ | |
| TDKSSGSPHSKA | 2306 | TVNGSGSPHSK | 2468 | MEWGHDSPH | 2630 | AIDGHDSPH | 2793 |
| QNQQ | | AQNQW | | KSGQNQQ | | KSGQNQL | |
| TIDGSGSPHSKA | 2307 | TINGSGSPHSKA | 2469 | CEYGHDSPH | 2631 | TIMGHDSPH | 2794 |
| QNQQ | | QLHH | | KSGQNQQ | | KSVQNQQ | |
| TVNGNGSPHSK | 2308 | TEQTSGSPHSKA | 2470 | RINGHDSPHK | 2632 | EVGGHDSPH | 2795 |
| AQNQH | | QNQQ | | SGQKQQ | | KSGQNQQ | |
| NTNGSGSPHSK | 2309 | TINGSGSPHSKA | 2471 | MEIGHDSPH | 2633 | TINGHDSPH | 2796 |
| AQNQQ | | QNII | | KSGQNQQ | | KSAQNLL | |
| TETHSGSPHSKA | 2310 | NSLGSGSPHSKA | 2472 | LEYGHDSPH | 2634 | TINASGSPHS | 2797 |
| QNQQ | | QNQQ | | KSGQNQQ | | KAINQQ | |
| TINGGGSPHSKA | 2311 | TIPMEGSPHSKA | 2473 | ADWGHDSPH | 2635 | AINGNGSPH | 2798 |
| LNQQ | | QNQQ | | KSGQNQQ | | KRGQNQQ | |
| TINGSGSPHSKA | 2312 | TINGSGSPHSKA | 2474 | WEIGHDSPHK | 2636 | SEMGHDSPH | 2799 |
| LHQH | | QGHH | | SGQNQQ | | KSGQNQQ | |
| TINGTGSPHSKA | 2313 | TDRTSGSPHSKA | 2475 | DIMGHDSPH | 2637 | AQQGHDSPH | 2800 |
| QNHQ | | QNQQ | | KSGQNQQ | | KSGQNQQ | |
| TINGSGSPHSKA | 2314 | TINGSGSPHSKA | 2476 | FEQGHDSPH | 2638 | AINGHDSPH | 2801 |
| QHRI | | QSKV | | KSGQNQQ | | KSGQSLQ | |
| TINGSGSPHSKA | 2315 | EVVGSGSPHSK | 2477 | MEFGHDSPH | 2639 | TINGSGSPHS | 2802 |
| QYIH | | AQNQQ | | KSGQNQQ | | KAPNQQ | |
| TENISGSPHSKA | 2316 | TINGSGSPHSKA | 2478 | CDQGHDSPH | 2640 | CGEGHDSPH | 2803 |
| QNQQ | | QLPS | | KSGQNQQ | | KSGQNQQ | |
| TIIGGGSPHSKA | 2317 | TINGSGSPHSKAI | 2479 | LPEGHDSPH | 2641 | TVNGHDSPH | 2804 |
| HNQQ | | GKQ | | KSGQNQQ | | KSAQNHQ | |
| TINGSGSPHSKA | 2318 | TEPTSGSPHSKA | 2480 | IENGHDSPHK | 2642 | TVNGHDSPH | 2805 |
| QKFE | | QNQQ | | SGQNQQ | | KSGQTQL | |
| TSNESGSPHSKA | 2319 | TVNGGGSPHSK | 2481 | MESGHDSPH | 2643 | TNNGHDSPH | 2806 |
| QNHQ | | SQNQQ | | KSGQNQQ | | KSGRNRQ | |
| TINGSGSPHSKA | 2320 | TINGSGSPHSKA | 2482 | AEIGHDSPHK | 2644 | TINGHDSPH | 2807 |
| QFPS | | QAIH | | SGQNQQ | | KYGQNEQ | |
| TERPSGSPHSKA | 2321 | TINGSGSPHSKA | 2483 | VEYGHDSPH | 2645 | TINGNGSPHS | 2808 |
| QNQQ | | QHGL | | KSGQNQQ | | KAPNLQ | |
| TINGNGSPHSKA | 2322 | SELGSGSPHSKA | 2484 | IINGHDSPHK | 2646 | SINGHDSPHK | 2809 |
| QNPL | | QNQQ | | SGLTQQ | | SQQNQQ | |
| SIKGNGSPHSKA | 2323 | TINGSGSPHSKA | 2485 | TSNGDDSPH | 2647 | TIGGHDSPH | 2810 |
| QNQQ | | QFMC | | KSGRNQQ | | KSGQNQQ | |
| TERMSGSPHSK | 2324 | TINVSGSPHSKA | 2486 | IEVGHDSPHK | 2648 | TINGHDSPH | 2811 |
| AQNQQ | | QGQQ | | SGQNQQ | | KSVQSKQ | |
| TERSSGSPHSKA | 2325 | TINGGGSPHSKA | 2487 | MEMGHDSPH | 2649 | ELVGHDSPH | 2812 |
| QNQQ | | QNQM | | KSGQNQQ | | KSGQNQQ | |
| TELHSGSPHSKA | 2326 | TVNGSGSPHSK | 2488 | AEVGHDSPH | 2650 | ELMGHDSPH | 2813 |
| QNQQ | | AQHLQ | | KSGQNQQ | | KSGQNQQ | |
| TELTSGSPHSKA | 2327 | TIRENGSPHSKA | 2489 | MDAGHDSPH | 2651 | TINGNDSPH | 2814 |
| QNQQ | | QNQQ | | KSGQNQQ | | KIGHNQQ | |
| TINGSGSPHSKA | 2328 | TINGSGSPHSKT | 2490 | VEWGHDSPH | 2652 | TIKGGGSPHS | 2815 |
| HNQQ | | QNHQ | | KSGQNQQ | | KAQDQQ | |
| TINGGGSPHSKA | 2329 | TINGSGSPHSKA | 2491 | AEQGHDSPH | 2653 | TVNGHDSPH | 2816 |
| QSQQ | | QPAR | | KSGQNQQ | | KSGQTQQ | |
| TINGSGSPHSKA | 2330 | TVNGSGSPHSK | 2492 | LEWGHDSPH | 2654 | TINGQDSPH | 2817 |
| QAIK | | AQSLQ | | KSGQNQQ | | KSGQNPL | |
| TENTSGSPHSKA | 2331 | TINGSGSPHSKS | 2493 | MELGHDSPH | 2655 | TVNASGSPH | 2818 |
| QNQQ | | QSQL | | KSGQNQQ | | SKAQNHQ | |
| TIDGSGSPHSKG | 2332 | TINGSASPHSKA | 2494 | METGHDSPH | 2656 | TINGHDSPH | 2819 |
| QNRQ | | HSQQ | | KSGQNQQ | | KSGRDQK | |
| NINGSGSPHSKA | 2333 | TWQNSGSPHSK | 2495 | MEAGHDSPH | 2657 | TINGHDSPH | 2820 |
| QSQQ | | AQNQQ | | KSGQNQQ | | KSVHNQQ | |
| TINGSVSPHGKA | 2334 | TINGSGSPHSKA | 2496 | IESGHDSPHK | 2658 | TINGHDSPH | 2821 |
| QNQL | | QDRQ | | SGQNQQ | | KSGQWKR | |
| TSNASGSPHSKA | 2335 | TINGSGSPHSKA | 2497 | MEVGHDSPH | 2659 | TIDGSGSPHS | 2822 |
| QNQL | | QMPS | | KSGQNQQ | | KAENRQ | |
| TEARSGSPHSKA | 2336 | TNNGGGSPHSK | 2498 | CEIGHDSPHK | 2660 | NEIGHDSPH | 2823 |
| QNQQ | | AQNLQ | | SGQNQQ | | KSGQNQQ | |
| TEKNSGSPHSKA | 2337 | TINGSGSPHSKA | 2499 | ATNGHDSPH | 2661 | AINGHDSPH | 2824 |
| QNQQ | | QGSL | | KSGLNQQ | | KSGQSQQ | |
| TANGSGSPHSK | 2338 | TEVTSGSPHSKA | 2500 | MDGGHDSPH | 2662 | IINGHDSPHK | 2825 |
| AQYQQ | | QNQQ | | KSGQNQQ | | SRQAQQ | |
| TVNGSGSPHSK | 2339 | SINGGGSPHSKA | 2501 | QEVGHDSPH | 2663 | TPNGHDSPH | 2826 |
| AQYQH | | QYQQ | | KSGQNQQ | | KSGQNQQ | |
| TINGSGSPHTKA | 2340 | TVIGSGSPHSKS | 2502 | ADQGHDSPH | 2664 | ITNGHDSPH | 2827 |
| QNPQ | | QNQQ | | KSGQNQQ | | KSGQTQQ | |
| TINGSGSPHSKG | 2341 | AVNVSGSPHSK | 2503 | TINGHESPHK | 2665 | TINGHDSPH | 2828 |
| QNPP | | AQNQQ | | SAQNHQ | | KSVQNHQ | |
| TIIGSGSPHSKAQ | 2342 | TVNGNGSPHSK | 2504 | TINGHDSPHK | 2666 | TINGHDSPH | 2829 |
| HQL | | SQNQQ | | SAQNQW | | KSKQNQQ | |
| TINGSGSPHSKA | 2343 | TDRNSGSPHSK | 2505 | NMNGHDSPH | 2667 | TINGHDSPH | 2830 |
| QSPP | | AQNQQ | | KSGQNTH | | KSAQNQL | |
| TIYGSGSPHSKA | 2344 | TINGSGSPHSKA | 2506 | EMGHDSPH | 2668 | TVNGHDSPH | 2831 |
| QNQQ | | QVPA | | KSGQNQQ | | KSGQTQP | |
| TINGSGSPHSKA | 2345 | GVLGSGSPHSK | 1279 | TINGHDSPHK | 2669 | TDQGHDSPH | 2832 |
| QAKL | | AQNQQ | | SGRRRQ | | KSGQNQQ | |
| TDKNSGSPHSK | 2346 | TLNGNGSPHSK | 2508 | ISNGHDSPHK | 2670 | TINGHDSPH | 2833 |
| AQNQQ | | AQNLQ | | SAQNQQ | | KLWINQQ | |
| TINGSGSPHSKT | 2347 | AINGSGSPHSKA | 2509 | TGNGHDSPH | 2671 | GINGPDSPHK | 2834 |
| KSQQ | | QDKQ | | KSGQYQQ | | SGQNQQ | |
| TINGSGSPHSKA | 2348 | TSNGSGSPHSKA | 2510 | TINGNYSPHK | 2672 | SEIGHDSPHK | 2835 |
| QDRP | | HYQQ | | IGQNQQ | | SGQNQQ | |
| TINGIGSPHSKA | 2349 | TINGSGSPHSKA | 2511 | TINGHDSPHK | 2673 | TINGHDSPH | 2836 |
| QNLG | | QVPS | | SRQNDQ | | KSVQKQL | |
| TINGSGSPHSKA | 2350 | TELRSGSPHSKA | 2512 | QQQGHDSPH | 2674 | TINGHPSPH | 2837 |
| QSQQ | | QNQQ | | KSGQNQQ | | WKGQNQQ | |
| TENLSGSPHSKA | 2351 | NINGSGSPHSKA | 2513 | HDWGHDSPH | 2675 | TVNGHDSPH | 2838 |
| QNQQ | | QNHQ | | KSGQNQQ | | KSGRNQL | |
| TINGSGSPHSKA | 2352 | TVNGGGSPHSK | 2514 | IEGGHDSPHK | 2676 | TIKGSGSPHS | 2839 |
| QAFH | | AQNHQ | | SGQNQQ | | KVQDQQ | |
| TINGSGSPHSKA | 2353 | TINGSGSPHSKA | 2515 | TFNRSGSPHS | 2677 | SEKGHDSPH | 2840 |
| QKQQ | | RGEQ | | KAQNQQ | | KSGQNQQ | |
| TFSGSGSPHSKA | 2354 | TINGGGSPHSKA | 2516 | AINGHDSPH | 2678 | WSAGHDSPH | 2841 |
| QNLQ | | QYQH | | KSGQIRQ | | KSGQNQQ | |
| AINGSGSPHSKA | 2355 | TEDLSGSPHSKA | 2517 | TINGHDSPHK | 2679 | ELAGHDSPH | 2842 |
| QNAQ | | QNQQ | | IGQNQH | | KSGQNQQ | |
| TESWSGSPHSKA | 2356 | TINGSGSPHSKA | 2518 | AINGYDSPH | 2680 | TINGHDSPH | 2843 |
| QNQQ | | PGQQ | | KSGQKQQ | | KMGRNQQ | |
| TTNGSGSPHSKA | 2357 | TIPKNGSPHSKA | 2519 | TESGHDSPH | 2681 | TINGHDSPH | 2844 |
| HNQL | | QNQQ | | KSGQNQQ | | KSGISIQ | |
| TVNGNGSPHSK | 2358 | TINGSGSPHSKA | 2520 | TINGHDSPHK | 2682 | TSNGHDSPH | 2845 |
| AQNHQ | | QSLQ | | SGQSVQ | | KSVQNLQ | |
| TEDKSGSPHSKA | 2359 | TINGSGSPHSKR | 2521 | TINGHESPHK | 2683 | QTQGHDSPH | 2846 |
| QNQQ | | LEQQ | | SGRSQQ | | KSGQNQQ | |
| TESASGSPHSKA | 2360 | TERGSGSPHSKA | 2522 | TINGHDSPHK | 2684 | TINGHDSPH | 2847 |
| QNQQ | | QNQQ | | SGQNKL | | KMAHNQQ | |
| TNNGSGSPHSK | 2361 | TVNGSGSPHSK | 2523 | TINGHDSPHK | 2685 | AINGSGSPHS | 2848 |
| AQNQQ | | APNQQ | | TGQNQQ | | KAQTQQ | |
| TSNGGGSPHSK | 2362 | TSNGSGSPHSKA | 2524 | TINGRGSPHK | 2686 | TINGHDSPH | 2849 |
| AQNLQ | | QNQS | | RGQNQQ | | KHGQNQQ | |
| TDKMSGSPHSK | 1242 | TINGSGSPHSKA | 1286 | TINGSGSPHT | 2687 | GADGHDSPH | 2850 |
| AQNQQ | | QKVI | | KAQNPP | | KSGQNQQ | |
| EVHGSGSPHSK | 2364 | TEGISGSPHSKA | 2526 | TINGQDSPHK | 2688 | VGEGHDSPH | 2851 |
| AQNQQ | | QNQQ | | SGQHQQ | | KSGQNQQ | |
| TINGSGSPHSKA | 1244 | TINGSGSPHSKA | 2527 | SINGHDSPHK | 2689 | ANEGHDSPH | 2852 |
| QKLN | | QNND | | SGQIQH | | KSGQNQQ | |
| TINGGGSPHSKS | 2366 | TINGSGSPHSKA | 2528 | AINGHDSPH | 2690 | TEAKSGSPHS | 2853 |
| QNQH | | QSVH | | KSGPRQQ | | KAQNQQ | |
| TVNGGGSPHSK | 2367 | TINGSGSPHSKA | 2529 | TVNGHDSPH | 2691 | TINGHDSPH | 2854 |
| AQSQQ | | QPLG | | KSGHTQQ | | KSVQSQQ | |
| TTNGSGSPHSKA | 2368 | TINKEGSPHSKA | 2530 | SINGHDSPHK | 2692 | TIPGSGSPHS | 2855 |
| QYQH | | QNQQ | | SGQRQH | | KAQNLQ | |
| TISGSGSPHSKA | 2369 | TCNASGSPHSK | 2531 | SLNGSGSPHT | 2693 | TINGHDSPH | 2856 |
| QYQH | | AQNQQ | | KAQNQQ | | KSGQTVC | |
| TESTSGSPHSKA | 2370 | AINGSGSPHSKA | 2532 | AINGHDSPH | 2694 | ELRGHDSPH | 2857 |
| QNQQ | | HNQE | | KSAQSQQ | | KSGQNQQ | |
| TINGSGSPHSKS | 2371 | TEGLSGSPHSKA | 2533 | SIYGHESPHK | 2695 | CQIGHDSPH | 2858 |
| QNVQ | | QNQQ | | SGQNQQ | | KSGQNQQ | |
| SINGSGSPHSKA | 2372 | TRDASGSPHSK | 2534 | TVNGHDSPH | 2696 | GVMGHDSPH | 2859 |
| QAQQ | | AQNQQ | | KSLQNQQ | | KSGQNQQ | |
| TVNGSGSPHSK | 2373 | TSNGSGSPHSKA | 2535 | TINGHGSPHS | 2697 | ACDGHDSPH | 2860 |
| AQNLQ | | QNLQ | | KAQNPQ | | KSGQNQQ | |
| TVRDSGSPHSKA | 2374 | TGNGSGSPHSK | 2536 | TSNGYDSPH | 2698 | TINGQDSPH | 2861 |
| QNQQ | | AQIQQ | | KSGQKQQ | | KSGQYQQ | |
| TFNASGSPHSKA | 2375 | TVNGGGSPHSK | 2537 | TVNGHDSPH | 2699 | TINGHDSPH | 2862 |
| PNQQ | | AQNLQ | | KSGRNQE | | KSGQQIM | |
| TDRMSGSPHSK | 1255 | TDRSSGSPHSKA | 2538 | TTNGHDSPH | 2700 | TINGHDSPH | 2863 |
| AQNQQ | | QNQQ | | KSGQTQL | | KSRQNEQ | |
| TINGSGSPHSKA | 2377 | TINGSGSPHSKA | 2539 | AINGHDSPH | 2701 | ASNGHDSPH | 2864 |
| QTPP | | QVRN | | KSEKNQQ | | KSGLNHQ | |
| TIKGSGSPHSKA | 2378 | TINGSGSPHSKA | 2540 | IINGRDSPHK | 2702 | TVNGHDSPH | 2865 |
| QNQQ | | PSNQ | | SGQDQQ | | KSGQSQP | |
| NHIGSGSPHSKA | 2379 | TINGSGSPHSKA | 2541 | TISGHDSPHK | 2703 | NELGHDSPH | 2866 |
| QNQQ | | QVGH | | TGHNQQ | | KSGQNQQ | |
| TINGSGSPHSKA | 2380 | NAIGSGSPHSKA | 2542 | SINGYDSPHK | 2704 | AAEGHDSPH | 2867 |
| QYQH | | QNQQ | | SGQTQQ | | KSGQNQQ | |
| TIPIDGSPHSKAQ | 1260 | AENGSGSPHSK | 2543 | TINGHESPHK | 2705 | GQNGHDSPH | 2868 |
| NQQ | | AQNQQ | | SGQTQQ | | KSGQNQQ | |
| TINGSGSPHSKA | 2382 | TINGSGSPHSKA | 2544 | TINGHDSPHK | 2706 | NEFGHDSPH | 2869 |
| QGQQ | | QRDI | | SGQSKQ | | KSGQNQQ | |
| TFNGSGSPHNK | 2383 | TINGSGSPHSKA | 2545 | AIIGHESPHK | 2707 | TSIGYDSPHK | 2870 |
| AQNHQ | | QMPN | | SGQNQQ | | SGQNQQ | |
| ESDGSGSPHSKA | 2384 | TVNGSGSPHSKS | 2546 | TINGHDSPHK | 2708 | TDNGHDSPH | 2871 |
| QNQQ | | QNQQ | | TGQNQP | | KSGQNLQ | |
| TINGSGSPHSKA | 2385 | TIPAIGSPHSKAQ | 1295 | AINGHDSPH | 2709 | TITGHDSPHK | 2872 |
| QPPA | | NQQ | | KSGQSPQ | | SRQDQQ | |
| TINGSGSPHSKA | 2386 | TINGSGSPHSKA | 1296 | TIKGNDSPHK | 2710 | AEHGHDSPH | 2873 |
| QERP | | RGLQ | | SVQNQQ | | KSGQNQQ | |
| TIKGSGSPHSKA | 2387 | TELGSGSPHSKA | 1297 | TEFGHDSPH | 2711 | TINGDDSPH | 2874 |
| QDLQ | | QNQQ | | KSGQNQQ | | KSGQKQL | |
| TDLKSGSPHSKA | 2388 | AETGSGSPHSKA | 1298 | TINGHDSPHK | 2712 | EILGHDSPHK | 2875 |
| QNQQ | | QNQQ | | SAQNYQ | | SGQNQQ | |
| TINGGGSPHSKA | 2389 | TINGSGSPHSKL | 1299 | TFNGSASPHS | 2713 | TIHGSGSPHS | 2876 |
| QNPP | | QKQQ | | KALNQQ | | KAQNQA | |
| TINGSGSPHSKA | 2390 | TINGSGSPHSKA | 1300 | TINGHESPHK | 2714 | AINGDDSPH | 2877 |
| QAMH | | PSLQ | | SAQNRQ | | KSGHNQQ | |
| TVPNSGSPHSKA | 2391 | TINGSGSPHSKA | 1301 | TTNGHDSPH | 2715 | TSNGHNSPH | 2878 |
| QNQQ | | QRDQ | | KSGQNQQ | | KSGQNQE | |
| TVIGSGSPHSKA | 2392 | TDVGSGSPHSK | 1302 | TIKGQDSPHK | 2716 | TINGHDSPH | 2879 |
| LNQQ | | AQNQQ | | IGQNQQ | | KSGQMIH | |
| TINGSGSPHSKA | 2393 | TINGSGSPHSKN | 1303 | TVNGHDSPH | 2717 | NAIGHDSPH | 2880 |
| QHPS | | RDQQ | | KSGQNHL | | KSGQNQQ | |
| TINGLGSPHSKS | 2394 | SINGSGSPHSKA | 1304 | SINGHDSPHK | 2718 | VINGHDSPH | 2881 |
| QNQQ | | PNLQ | | SGQYQH | | KSGRNHQ | |
| TINGTGSPHSKA | 2395 | TINGSGSPHSKA | 1305 | TINGNDSPHK | 2719 | TITGHDSPHK | 2882 |
| QNQQ | | QAKG | | SVQNHQ | | SVQNRQ | |
| TINGSGSPHSKA | 2396 | TVNGSGSPHSK | 1306 | TITGHDSPHK | 2720 | TINGHDSPH | 2883 |
| PGLQ | | AQDKQ | | SGQNQW | | KSGQKMN | |
| TINGSGSPHSKA | 2397 | TINGGGSPHSKA | 1307 | TNNGHDSPH | 2721 | TIHGHDSPH | 2884 |
| QGIR | | QNPQ | | KSVQNQH | | KSGQSQQ | |
| TESHSGSPHSKA | 2398 | TINGSGSPHSKA | 1308 | TIDGHDSPHK | 2722 | TEIGHDSPHK | 2885 |
| QNQQ | | QSAH | | SGQNQH | | SGQNQQ | |
| TINGSGSPHSKA | 2399 | TINGNGSPHSKS | 1309 | TVNGHDSPH | 2723 | TINGHDSPH | 2886 |
| QAPA | | QNQH | | KSGQTRQ | | KSGQYQH | |
| TINGSGSPHSKS | 2400 | TVPTSGSPHSKA | 1310 | TVNGHDSPH | 2724 | NCLGSGSPH | 2403 |
| QSQQ | | QNQQ | | KSGQNLH | | SKAQNQQ | |
| AEHGSGSPHSK | 2401 | TIDGSGSPHSKS | 1311 | AISGHDSPHK | 2725 | AINRSGSPHS | 1313 |
| AQNQQ | | QNHQ | | SGLNQQ | | KAQDQQ | |
| TEDRSGSPHSKA | 2402 | TDVKSGSPHSK | 1312 | AINGHDSPH | 2726 | TITGHDSPHK | 2727 |
| QNQQ | | AQNQQ | | KSAQNQE | | SGQHLQ | |
| GSGSPHSKAQN | 2887 | GSGSPHSKAQS | 2936 | GSGSPHSKA | 2985 | GSGSPHSKA | 3034 |
| RHT | | QQT | | QRMST | | QNNDQ | |
| GSGSPHSKVQN | 2888 | ASGSPHSKAQN | 2937 | GSGSPHSKA | 2986 | GSGSPHSKA | 3035 |
| QQT | | QLT | | QGILT | | QSVHT | |
| MSGSPHSKAQN | 2889 | RSGSPHSKAQN | 2938 | GSGSPHSKA | 2987 | GSGSPHSKA | 3036 |
| QQT | | QQT | | QDRQT | | QPLGT | |
| GSGSPHSKARD | 2890 | GSGSPHSKAQY | 2939 | GSGSPHSKA | 2988 | GSGSPHSKA | 3037 |
| QQT | | QHT | | RDWQT | | HNQET | |
| GSGSPHSKAPNL | 2891 | GSGSPHTKAQN | 2940 | GSGSPHSKA | 2989 | GSGSPHSKA | 3038 |
| QT | | PQS | | QNTHD | | QNLQI | |
| TSGSPHSKAQN | 2892 | GSGSPHSKGQN | 2941 | GSGSPHSKAP | 2990 | GSGSPHSKA | 3039 |
| QQT | | PPT | | NLQI | | QIQQT | |
| GSGSPHSKAHV | 2893 | GSGSPHSKAQH | 2942 | GSGSPHSKA | 2991 | GSGSPHSKA | 3040 |
| RQT | | QLT | | QERST | | QVRNT | |
| GSGSPHSKAPNQ | 2894 | GSGSPHSKAQSP | 2943 | GSGSPHSKA | 2992 | GSGSPHSKA | 3041 |
| HT | | PT | | QNYQT | | PSNQT | |
| ISGSPHSKAQNQ | 2895 | GSGSPHSKAQA | 2944 | GSGSPHSKA | 2993 | GSGSPHSKA | 3042 |
| QT | | KLT | | QRTCT | | QVGHT | |
| GSGSPHSKTQSQ | 2896 | GSGSPHSKTKSQ | 2945 | GSGSPHSKA | 2994 | GSGSPHSKA | 3043 |
| QT | | QT | | QIGHT | | QRDIT | |
| GSGSPHSKAQN | 3032 | GSGSPHSKAQD | 2946 | GSGSPHSKA | 2995 | GSGSPHSKA | 3044 |
| QST | | RPT | | QGAIT | | QMPNT | |
| ESGSPHSKAQN | 2898 | GSGSPHSKAQS | 2947 | GSGSPHSKA | 2996 | GSGSPHSKA | 3045 |
| QQI | | QQL | | QVPPT | | RGLQT | |
| GSGSPHSKAQPA | 2899 | GSGSPHSKAQA | 2948 | GSGSPHSKA | 2997 | GSGSPHSKL | 3046 |
| KT | | FHT | | QVQQI | | QKQQT | |
| SSGSPHSKAQNQ | 2900 | GSGSPHSKAQK | 2949 | GSGSPHSKAL | 2998 | GSGSPHSKA | 3047 |
| QT | | QQD | | MRQT | | PSLQT | |
| GSGSPHSKSQTQ | 2901 | GSGSPHSKAQN | 2950 | GSGSPHSKA | 2999 | GSGSPHSKA | 3048 |
| QN | | AQT | | QYSVT | | QRDQT | |
| ASGSPHSKAQN | 2902 | WSGSPHSKAQN | 2951 | GSGSPHSKVP | 3000 | GSGSPHSKN | 3049 |
| QQT | | QQT | | NLQT | | RDQQT | |
| GSGSPHSKAQN | 2903 | GSGSPHSKAHN | 2952 | GSGSPHSKA | 3001 | GSGSPHSKA | 3050 |
| LAT | | QLT | | QSQQI | | QAKGT | |
| GSGSPHSKSQNQ | 2904 | GSGSPHSKAQN | 2953 | GSGSPHSKA | 3002 | GSGSPHSKA | 3051 |
| LT | | QQY | | QAITT | | QSAHT | |
| NSGSPHSKAQN | 2905 | GSGSPHSKAQK | 2954 | GSGSPHSKA | 3003 | GSGSPHSKS | 3052 |
| QQT | | LNT | | QKTLT | | QNHQT | |
| GSGSPHSKAQG | 2906 | GSGSPHSKSQN | 2955 | GSGSPHSKA | 3004 | RSGSPHSKA | 3053 |
| QQT | | VQT | | QNQWT | | QDQQT | |
| VSGSPHSKAQN | 903 | GSGSPHSKAQA | 2956 | GSGSPHSKA | 3005 | GSGSPHSKA | 3054 |
| QQT | | QQT | | QLHHT | | QSTMT | |
| GSGSPHSKALNR | 2908 | GSGSPHSKAQN | 2957 | GSGSPHSKA | 3006 | GSGSPHSKA | 3055 |
| QS | | LQA | | QNIII | | QREMT | |
| LSGSPHSKAQN | 2909 | DSGSPHSKAQN | 2958 | GSGSPHSKA | 3007 | GSGSPHSKA | 3056 |
| QQT | | QQT | | QGHHT | | QYRAT | |
| GSGSPHSKAHN | 2910 | ASGSPHSKAPN | 2959 | GSGSPHSKA | 3008 | GSGSPHSKN | 3057 |
| QQT | | QQT | | QSKVT | | QWQQT | |
| GSGSPHSKTQN | 2911 | GSGSPHSKAQTP | 2960 | GSGSPHSKA | 3009 | GSGSPHSKA | 3058 |
| QQT | | PT | | QLPST | | QRMNT | |
| GSGSPHSKAQNP | 2912 | GSGSPHSKAQY | 2961 | GSGSPHSKAI | 3010 | GSGSPHAKA | 3059 |
| PT | | QHA | | GKQT | | QNHQT | |
| GSGSPHSKAQN | 2913 | GSGSPHSKAQG | 2962 | GSGSPHSKA | 3011 | GSGSPHSKA | 3060 |
| LQT | | QQA | | QAIHT | | GDSQT | |
| GSGSPHSKAQY | 2914 | GSGSPHNKAQN | 2963 | GSGSPHSKA | 3012 | GSGSPHSKL | 3061 |
| QQT | | HQT | | QHGLT | | KSQQT | |
| GSGSPHSKAQD | 2915 | GSGSPHSKAQPP | 2964 | GSGSPHSKA | 3013 | GSGSPHSKA | 3062 |
| QET | | AT | | QFMCT | | QKIST | |
| KSGSPHSKAQN | 2916 | GSGSPHSKAQE | 2965 | VSGSPHSKA | 3014 | GSGSPHSKA | 3063 |
| QQT | | RPT | | QGQQT | | PSMQT | |
| GSGSPHSKAQN | 913 | GSGSPHSKAQD | 2966 | GSGSPHSKA | 3015 | GSGSPHSKA | 3064 |
| HQT | | LQT | | QHLQT | | SPRQT | |
| GSGSPHSKAQD | 2918 | GSGSPHSKAQA | 2967 | GSGSPHSKTQ | 3016 | GSGSPHSKR | 3065 |
| QQT | | MHT | | NH?N | | MEQQT | |
| GSGSPHSKGHW | 2919 | GSGSPHSKALN | 2968 | GSGSPHSKA | 3017 | GSGSPHSKA | 3066 |
| QQT | | QQT | | QPART | | QYQNT | |
| GSGSPHSKAPNQ | 2920 | GSGSPHSKAQH | 2969 | GSGSPHSKA | 3018 | RSGSPHSKA | 3067 |
| QT | | PST | | QSLQT | | QNQQI | |
| GSGSPHSKAQN | 2921 | GSGSPHSKAPGL | 2970 | GSGSPHSKSQ | 3019 | GSGSPHTKA | 3068 |
| QLI | | QT | | SQLT | | QNPPT | |
| GSGSPHSKRPEQ | 2922 | GSGSPHSKAQGI | 2971 | GSGSPHSKA | 3020 | GSGSPHTKA | 3069 |
| QT | | RT | | QDRQS | | QNQQT | |
| GSGSPHSKAQRT | 2923 | GSGSPHSKAQA | 2972 | GSGSPHSKA | 3021 | ASGSPHSKA | 3070 |
| MT | | PAT | | QMPST | | QHQQT | |
| GSGSPHSKAQN | 2924 | GSGSPHSKSQSQ | 2973 | GSGSPHSKA | 3022 | ASGSPHSKAI | 3071 |
| QQH | | QI | | QGSLT | | NQQT | |
| HSGSPHSKAQN | 2925 | GSGSPHSKAQIP | 2974 | GSGSPHSKSQ | 3023 | GSGSPHSKA | 3072 |
| QQT | | PT | | NQQT | | PNQQH | |
| GSGSPHSKALH | 2926 | GSGSPHSKAQT | 2975 | GSGSPHSKA | 3024 | ASGSPHSKA | 3073 |
| QHT | | QLT | | QVPAT | | QNHQT | |
| GSGSPHSKAQH | 2927 | GSGSPHSKAQA | 2976 | GSGSPHSKA | 3025 | GSGSPHSKA | 3074 |
| RIT | | PST | | QDKQT | | ENRQT | |
| GSGSPHSKAQYI | 2928 | GSGSPHSKGQN | 2977 | GSGSPHSKA | 3026 | GSGSPHSKV | 3075 |
| HT | | QQT | | HYQQT | | QDQQT | |
| GSGSPHSKAQKF | 2929 | ASGSPHSKAHN | 2978 | GSGSPHSKA | 3027 | GSGSPHSKA | 3076 |
| ET | | QQT | | QVPST | | QTQQA | |
| ESGSPHSKAQN | 2930 | GSGSPHSKAQN | 2979 | GSGSPHSKA | 3028 | GSGSPHSKA | 3077 |
| HQT | | TYA | | RGEQT | | QNQAT | |
| GSGSPHSKAQFP | 2931 | GSGSPHSKSQN | 2980 | GSGSPHSKAP | 3029 | GSGSPHSKG | 2935 |
| ST | | QHI | | GQQT | | QNRQT | |
| PSGSPHSKAQNQ | 2932 | GSGSPHSKAQN | 2981 | GSGSPHSKA | 3030 | GSGSPHSKA | 2984 |
| QT | | HLT | | QSLQI | | QLQQT | |
| GSGSPHSKAHN | 2933 | GSGSPHSKAQIG | 2982 | GSGSPHSKRL | 3031 | GSGSPHSKA | 3033 |
| QQR | | MT | | EQQT | | QKVIT | |
| GSGSPHSKAQAI | 2934 | GSGSPHSKALN | 2983 | GHDSPHKHG | 3160 | GHDSPHKSG | 3202 |
| KT | | KQT | | QNHQT | | QWKRT | |
| GHDSPHKSGQIR | 3078 | GHDSPHKSGQN | 3119 | LHDSPHKSG | 3161 | GHDSPHKSG | 3203 |
| QT | | QHA | | QNQQT | | QSQQI | |
| GHDSPHKIGQN | 3079 | GHDSPHKSGQT | 3120 | GHDSPHKSV | 3162 | GHDSPHKSR | 3204 |
| QHA | | RQT | | QNKQT | | QAQQT | |
| GYDSPHKSGQK | 3080 | GHDSPHKSGQN | 3121 | GQVSPHKSG | 3163 | GHDSPHKSV | 3205 |
| QQT | | LHT | | QNQQT | | QNHQI | |
| GHDSPHKSGQN | 201 | GHDSPHKSGLN | 3122 | GHDSPHKSG | 3164 | GHDSPHKSK | 3206 |
| QQT | | QQT | | QRQLT | | QNQQA | |
| GHDSPHKSGQS | 3082 | GHDSPHKSAQN | 3123 | GHDSPHKIG | 3165 | GHDSPHKSA | 3207 |
| VQT | | QET | | QNQLT | | QNQLN | |
| GHESPHKSGRSQ | 3083 | GHDSPHKSGQH | 3124 | GHDSPHKSG | 3166 | GHDSPHKSG | 3208 |
| QT | | LQT | | QIIVT | | QTQPT | |
| GHDSPHKSGQN | 3084 | GHDSPHKSRLN | 3125 | IHDSPHKSGQ | 3167 | FHDSPHKSG | 3209 |
| KLE | | QPT | | NQQT | | QNQQT | |
| GHDSPHKTGQN | 3085 | GHDSPHKSGQK | 3126 | GYDSPHKSG | 3168 | GHDSPHKLW | 3210 |
| QQK | | QQT | | QKQST | | INQQT | |
| GRGSPHKRGQN | 3086 | GQDSPHKSGQN | 3127 | MHDSPHKSG | 3169 | GPDSPHKSG | 3211 |
| QQT | | QDT | | QNQQT | | QNQQT | |
| GHDNPHKSGQN | 3087 | GHDSPHKSGRN | 3128 | GDDSPHKSV | 3170 | GHDSPHKSV | 3212 |
| QQT | | QQT | | QNQQT | | QKQLT | |
| GQDSPHKSGQH | 3088 | GHDSPHKSGQN | 3129 | GHDSPHKSV | 3171 | GHDSPHKSG | 3213 |
| QQA | | LLT | | QSHQT | | RNQLA | |
| GHDSPHKSGQIQ | 3089 | GHDSPHKSGQL | 3130 | GHDSPHKSG | 3172 | VHDSPHKSG | 3214 |
| HT | | VIT | | QFVVT | | QNQQS | |
| GHDSPHKSGPR | 3090 | GHDSPHKSRQS | 3131 | GHDSPHKSR | 3173 | GHDSPHKMG | 3215 |
| QQT | | QQT | | QNLQT | | RNQQS | |
| GHDSPHKSGHT | 3091 | GHDSPHKSGRT | 3132 | GHNSPHKSG | 3174 | GHDSPHKSGI | 3216 |
| QQT | | QET | | QNQET | | SIQT | |
| GHDSPHKSGQR | 3092 | GHDSPHKSVQT | 3133 | GHDSPHKSA | 3175 | VHDSPHKSG | 3217 |
| QHT | | HQT | | QNQQI | | QNQQT | |
| GHDSPHKSAQS | 3093 | GHDSPHKSGQN | 3134 | NHDSPHKSG | 3176 | GHDSPHKSV | 3218 |
| QQT | | QPA | | QNQQT | | QNLQT | |
| GHESPHKSGQN | 3094 | KHDSPHKSGQN | 3135 | GHDSPHKSG | 3177 | GHDSPHKMA | 3219 |
| QQS | | QQT | | QSQPT | | HNQQT | |
| GHDSPHKSLQN | 3095 | GHDSPHKSGQT | 3136 | GHESPHKSG | 3178 | GHDSPHKHG | 3220 |
| QQT | | QQT | | QNEQT | | QNQQN | |
| GHDSPHKSGRN | 3096 | GPDSPHKIGQNQ | 3137 | GHDSPHKSA | 3179 | GHDSPHKSV | 3221 |
| QET | | QS | | QNQQT | | QSQQS | |
| GHDSPHKSGQT | 3097 | GHDSPHKSVQN | 3138 | GHDSPHKSG | 3180 | GHDSPHKSG | 3222 |
| QLT | | QQT | | QNQLT | | QTVCT | |
| GHDSPHKSEKN | 3098 | GHDSPHKSRQD | 3139 | GHDSPHKSA | 3181 | GQDSPHKSG | 3223 |
| QQT | | QHT | | QNLLT | | QYQQI | |
| GRDSPHKSGQD | 3099 | GPDSPHKSGQK | 3140 | THDSPHKSG | 3182 | GHDSPHKSG | 3224 |
| QQT | | QQT | | QNQQT | | QQIMT | |
| GHDSPHKTGHN | 3100 | GHDSPHKSRQS | 3141 | GNGSPHKRG | 3183 | GHDSPHKSR | 3225 |
| QQT | | QHT | | QNQQT | | QNEQS | |
| AHDSPHKSGQN | 3101 | GHDSPHKSVQN | 3142 | GHDSPHKSG | 3184 | GHDSPHKSG | 3226 |
| QLT | | QLT | | QSLQT | | LNHQT | |
| GYDSPHKSGQT | 3102 | AHDSPHKSGQN | 3143 | GHDSPHKSA | 3185 | GYDSPHKSG | 3227 |
| QQT | | QQT | | QNHQT | | QNQQT | |
| GHESPHKSGQT | 3103 | GQDSPHKSGQN | 3144 | GHDSPHKSG | 3186 | GHDSPHKSG | 3228 |
| QQI | | QQS | | RNRQT | | QNLQT | |
| GHDSPHKSGQS | 3104 | GHDSPHKSGRN | 3145 | EHDSPHKSG | 3187 | GHDSPHKSR | 3229 |
| KQA | | QQI | | QNQQT | | QDQQT | |
| GHESPHKSGQN | 3105 | GYDSPHKSGQY | 3146 | GHDSPHKYG | 3188 | GDDSPHKSG | 3230 |
| QQT | | QHT | | QNEQT | | QKQLT | |
| GHDSPHKTGQN | 3106 | GHDSPHKSRQN | 3147 | RHDSPHKSG | 3189 | GDDSPHKSG | 3231 |
| QPP | | QQT | | HNQQT | | HNQQT | |
| GHDSPHKSGQSP | 3107 | GHDSPHKSWVR | 3148 | GHDSPHKSQ | 3190 | GHDSPHKSG | 3232 |
| QT | | QQT | | QNQQT | | QMIHT | |
| GNDSPHKSVQN | 3108 | GHESPHKSGQN | 3149 | GHDSPHKSG | 3191 | GHDSPHKSG | 3233 |
| QQT | | QHS | | QNQQI | | RNHQS | |
| GHDSPHKSAQN | 3109 | GHDSPHKIGHN | 3150 | QHDSPHKSG | 3192 | GHDSPHKSV | 3234 |
| YQT | | QQT | | QNQQT | | QNRQT | |
| AHDSPHKIGQN | 3110 | WHDSPHKSGQN | 3151 | RHDSPHKIVQ | 3193 | GHDSPHKSG | 3235 |
| HQT | | QQT | | NQQT | | QKMNT | |
| GHESPHKSAQN | 3111 | RHDSPHKSGQN | 3152 | YHDSPHKSG | 3194 | GHDSPHKSG | 3236 |
| RQT | | QQT | | QNQQT | | QSQQN | |
| GHDSPHKSGQN | 3112 | GHDSPHKSNAW | 3153 | GHDSPHKSV | 3195 | GHDSPHKSG | 3237 |
| QQG | | QQT | | QSKQT | | QYQHA | |
| GQDSPHKIGQN | 3113 | GHDSPHKSGQS | 3154 | GNDSPHKIG | 3196 | GHDSPHKSG | 3117 |
| QQT | | VPT | | HNQQT | | QNQWT | |
| GHDSPHKSGQN | 3114 | GHESPHKSGQNI | 3155 | HHDSPHKSG | 3197 | GHDSPHKRG | 3158 |
| HLT | | QP | | QNQQT | | PDQQS | |
| GHDSPHKSGQY | 3115 | GHDSPHKSVQN | 3156 | GHDSPHKSG | 3198 | GHDSPHKSG | 3200 |
| QHT | | HLN | | QTQQI | | RDQKT | |
| GNDSPHKSVQN | 3116 | GHDSPHKIGLDQ | 3157 | GQDSPHKSG | 3199 | GHDSPHKSV | 3201 |
| HQT | | QT | | QNPLT | | HNQQN | |
| GHDSPHKSVQN | 3118 | GHDSPHKSGES | 3159 | KTISKRGSPH | 4098 | KGLGGSGSP | 4099 |
| QHT | | QQT | | SKAQNQQT | | HSKAQNQQT | |
| KTINGHDSPHSK | 4100 | KTINGSGSPHSK | 4196 | KEIYGSGSPH | 4292 | KTINGSGSPH | 4388 |
| AQNLQT | | TCIQQT | | SKAQNQQT | | KRGQKQQT | |
| KTINGHDSPHSK | 4101 | KTINGSGSPHSK | 4197 | KELSGSGSPH | 4293 | KTINGSGSPH | 4389 |
| AQNQQI | | WLTQQT | | SKAQNQQT | | KRGQNQET | |
| KTINGSGSPHFT | 4102 | KTINGSGSPHSK | 4198 | KETIGSGSPH | 4294 | KTINGSGSPH | 4390 |
| RQNQQT | | WVVQQT | | SKAQNQQT | | KRGQNQLT | |
| KTINGSGSPHSL | 4103 | KTINGSGSPHSK | 4199 | KEVLGSGSP | 4295 | KTINGSGSPH | 4391 |
| PWNQQT | | YRLQQT | | HSKAQNQQT | | KRGRNQQT | |
| KTINGHDSPHSK | 4104 | KTINGSGSPHSK | 4200 | KFALGHDSP | 4296 | KTINGSGSPH | 4392 |
| AQNHQT | | YSKQQT | | HKSGQKQQT | | KSGGNQQT | |
| KTMNGHDSPHS | 4105 | KTINGSGSPHSK | 4201 | KIINGHDSPH | 4297 | KTINGSGSPH | 4393 |
| KAQNQQT | | YSRQQT | | KSGQNLVL | | KSGHNQET | |
| KPYKGSGSPHSK | 4106 | KTINGSGSPHSL | 4202 | KIINGHDSPH | 4298 | KTINGSGSPH | 4394 |
| AQNQQT | | KRNQQT | | KSGQRNYT | | KSGHNQLT | |
| KRLWGSGSPHS | 4107 | KTINGSGSPHSL | 4203 | KIINGHDSPH | 4299 | KTINGSGSPH | 4395 |
| KAQNQQT | | WFNQQT | | SKAQNQQT | | KSGHNQQN | |
| KRMRGSGSPHS | 4108 | KTINGSGSPHSL | 4204 | KLNPGHDSP | 4300 | KTINGSGSPH | 4396 |
| KAQNQQT | | WPNQQT | | HKSGQTQQT | | KSGLNQLT | |
| KRTYGSGSPHSK | 4109 | KTINGSGSPHSL | 4205 | KLNRGHDSP | 4301 | KTINGSGSPH | 4397 |
| AQNQQT | | WTNQQT | | HKSGQNQQS | | KSGPNQQT | |
| KTINCLRSPHSK | 4110 | KTINGSGSPHSM | 4206 | KLSSGHDSPH | 4302 | KTINGSGSPH | 4398 |
| AQNQQT | | RRNQQT | | KSGQNQQN | | KSGQGQQT | |
| KTINFSRSPHSK | 4111 | KTINGSGSPHSP | 4207 | KNINGHDSP | 4303 | KTINGSGSPH | 4399 |
| AQNQQT | | CLNQQT | | HSKAQNQQT | | KSGQHLQT | |
| KTINGLRSPHFK | 4112 | KTINGSGSPHSQ | 4208 | KNNDGSGSP | 4304 | KTINGSGSPH | 4400 |
| AQNQQT | | WQNQQT | | HSKAQNQQT | | KSGQHQQT | |
| KTINGNRSPHNK | 4113 | KTINGSGSPHSR | 4209 | KNVMGSGSP | 4305 | KTINGSGSPH | 4401 |
| AQNQQT | | CANQQT | | HSKAQNQQT | | KSGQKHQT | |
| KTINGPRSPHYK | 4114 | KTINGSGSPHSRI | 4210 | KPINGHDSPH | 4306 | KTINGSGSPH | 4402 |
| AQNQQT | | RNQQT | | KSGQNKLS | | KSGQKQQS | |
| KTINGQASPHW | 4115 | KTINGSGSPHSR | 4211 | KPINGHDSPH | 4307 | KTINGSGSPH | 4403 |
| KAQNQQT | | KSNQQT | | KSGQNLSS | | KSGQNEQT | |
| KTINGRCSPHSK | 4116 | KTINGSGSPHSR | 4212 | KPINGHDSPH | 4308 | KTINGSGSPH | 4404 |
| AQNQQT | | LWNQQT | | SKAQNQQT | | KSGQNHQT | |
| KTINGRHSPHSK | 4117 | KTINGSGSPHSR | 4213 | KRINGHDSPH | 4309 | KTINGSGSPH | 4405 |
| AQNQQT | | RFNQQT | | SKAQNQQT | | KSGQNKQT | |
| KTINGRKSPHRK | 4118 | KTINGSGSPHSR | 4214 | KSCSGHDSP | 4310 | KTINGSGSPH | 4406 |
| AQNQQT | | RPNQQT | | HKSGQNQQS | | KSGQNKTS | |
| KTINGRLSPHW | 4119 | KTINGSGSPHSR | 4215 | KSINGHDSPH | 4311 | KTINGSGSPH | 4407 |
| KAQNQQT | | SCNQQT | | KSGQNLAS | | KSGQNQEA | |
| KTINGRLSPHYK | 4120 | KTINGSGSPHSR | 4216 | KSINGHDSPH | 4312 | KTINGSGSPH | 4408 |
| AQNQQT | | SKNQQT | | KSGQNLFL | | KSGQNQET | |
| KTINGRPSPHMK | 4121 | KTINGSGSPHSR | 4217 | KSINGHDSPH | 4313 | KTINGSGSPH | 4409 |
| AQNQQT | | TKNQQT | | KSGQNLLM | | KSGQNQKT | |
| KTINGRSSPHWK | 4122 | KTINGSGSPHSR | 4218 | KSINGHDSPH | 4314 | KTINGSGSPH | 4410 |
| AQNQQT | | WLNQQT | | KSGQNLLQ | | KSGQNQQI | |
| KTINGRWSPHSK | 4123 | KTINGSGSPHSS | 4219 | KSINGHDSPH | 4315 | KTINGSGSPH | 4411 |
| AQNQQT | | VCNQQT | | KSGQNSLG | | KSGQNQQR | |
| KTINGSGSPHAP | 4124 | KTINGSGSPHSS | 4220 | KSINGHDSPH | 4316 | KTINGSGSPH | 4412 |
| CQNQQT | | WRNQQT | | KSGQNTLQ | | KSGQNQRT | |
| KTINGSGSPHA | 4125 | KTINGSGSPHSV | 4221 | KSINGHDSPH | 4317 | KTINGSGSPH | 4413 |
| WAQNQQT | | CQNQQT | | KSSSNQQT | | KSGQNQYT | |
| KTINGSGSPHCM | 4126 | KTINGSGSPHSV | 4222 | KSINGHDSPF | 4318 | KTINGSGSPH | 4414 |
| RQNQQT | | LCNQQT | | KYKLNQQT | | KSGQRQQT | |
| KTINGSGSPHFC | 4127 | KTINGSGSPHSV | 4223 | KSINGSGSPH | 4319 | KTINGSGSPH | 4415 |
| SQNQQT | | RRNQQT | | KSGQKQQT | | KSGQSQQT | |
| KTINGSGSPHFL | 4128 | KTINGSGSPHSV | 4224 | KSINGSGSPH | 4320 | KTINGSGSPH | 4416 |
| FQNQQT | | SCNQQT | | KSGQNQQT | | KSGQYQRT | |
| KTINGSGSPHFW | 4129 | KTINGSGSPHSW | 4225 | KSINGSGSPH | 4321 | KTINGSGSPH | 4417 |
| AQNQQT | | ALNQQT | | SKAQGLST | | KSGRNQQA | |
| KTINGSGSPHLC | 4130 | KTINGSGSPHSW | 4226 | KSINGSGSPH | 4322 | KTINGSGSPH | 4418 |
| AQNQQT | | ITNQQT | | SKAQLLGT | | KSRHNQQT | |
| KTINGSGSPHLR | 4131 | KTINGSGSPHSW | 4227 | KSINGSGSPH | 4323 | KTINGSGSPH | 4419 |
| YQNQQT | | PMNQQT | | SKTSWQQT | | KSRQYQQT | |
| KTINGSGSPHLY | 4132 | KTINGSGSPHSW | 4228 | KSMNGHDSP | 4324 | KTINGSGSPH | 4420 |
| YQNQQT | | RSNQQT | | HSKAQNQQT | | RKAQAPGT | |
| KTINGSGSPHPL | 4133 | KTINGSGSPHSY | 4229 | KSTLGSGSPH | 4325 | KTINGSGSPH | 4421 |
| CQNQQT | | FLNQQT | | SKAQNQHT | | SKAAMKQT | |
| KTINGSGSPHRI | 4134 | KTINGSGSPHSY | 4230 | KSTLGSGSPH | 4326 | KTINGSGSPH | 4422 |
| RQNQQT | | TYNQQT | | SKAQNQQN | | SKAGRQQT | |
| KTINGSGSPHRL | 4135 | KTINGSGSPHSY | 4231 | KSTVGSGSPH | 4327 | KTINGSGSPH | 4423 |
| FQNQQT | | WQNQQT | | SKAQTQQT | | SKAGRTQT | |
| KTINGSGSPHSC | 4136 | KTINGSGSPHTL | 4232 | KTCKESGSPH | 4328 | KTINGSGSPH | 4424 |
| GQNQQT | | CQNQQT | | SKAQNQQT | | SKAKSNQT | |
| KTINGSGSPHSC | 4137 | KTINGSGSPHWL | 4233 | KTCKGSGSP | 4329 | KTINGSGSPH | 4425 |
| LRNQQT | | RQNQQT | | HSKAQNQQT | | SKALKTQT | |
| KTINGSGSPHSC | 4138 | KTINGSGSPHWP | 4234 | KTCKSSGSPH | 4330 | KTINGSGSPH | 4426 |
| LSNQQT | | SQNQQT | | SKAQNQQT | | SKAPRTQT | |
| KTINGSGSPHSC | 4139 | KTINGSGSPHYL | 4235 | KTDMGSGSP | 4331 | KTINGSGSPH | 4427 |
| RLNQQT | | RQNQQT | | HSKAQNQQT | | SKAQAART | |
| KTINGSGSPHSC | 4140 | KTINGSGSPHYT | 4236 | KTDNGIGSPH | 4332 | KTINGSGSPH | 4428 |
| SLNQQT | | RQNQQT | | SKAQNQQT | | SKAQAILT | |
| KTINGSGSPHSK | 4141 | KTINGSLSPHLW | 4237 | KTEGGSGSP | 4333 | KTINGSGSPH | 4429 |
| ACTLQT | | AQNQQT | | HSKAQNQQT | | SKAQCRGT | |
| KTINGSGSPHSK | 4142 | KTINGSPSPHCQ | 4238 | KTEHHSGSP | 4334 | KTINGSGSPH | 4430 |
| AFRAQT | | AQNQQT | | HSKAQNQQT | | SKAQGLRT | |
| KTINGSGSPHSK | 4143 | KTINGSRSPHLC | 4239 | KTEKDSGSP | 4335 | KTINGSGSPH | 4431 |
| AIRKQT | | AQNQQT | | HSKAQNQQT | | SKAQKGVL | |
| KTINGSGSPHSK | 4144 | KTINGSRSPHWR | 4240 | KTELGHDSP | 4336 | KTINGSGSPH | 4432 |
| AQASRT | | AQNQQT | | HKRGQNQQT | | SKAQKSNT | |
| KTINGSGSPHSK | 4145 | KTINGSVSPHWL | 4241 | KTESVSGSPH | 4337 | KTINGSGSPH | 4433 |
| AQFELT | | AQNQQT | | SKAQNQQT | | SKAQNNKF | |
| KTINGSGSPHSK | 4146 | KTINGTFSPHRK | 4242 | KTETNSGSPH | 4338 | KTINGSGSPH | 4434 |
| AQIVIT | | AQNQQT | | SKAQNQQT | | SKAQNRRT | |
| KTINGSGSPHSK | 4147 | KTINGWTSPHR | 4243 | KTETYSGSPH | 4339 | KTINGSGSPH | 4435 |
| AQLART | | KAQNQQT | | SKAQNQQT | | SKAQPKQT | |
| KTINGSGSPHSK | 4148 | KTINRGISPHSK | 4244 | KTEWLSGSP | 4340 | KTINGSGSPH | 4436 |
| AQLQRT | | AQNQQT | | HSKAQNQQT | | SKAQRAPT | |
| KTINGSGSPHSK | 4149 | KTINTVRSPHSK | 4245 | KTFNGSGSPH | 4341 | KTINGSGSPH | 4437 |
| AQNARR | | AQNQQT | | KSGQNQQT | | SKAQREHT | |
| KTINGSGSPHSK | 4150 | KTKLRSGSPHSK | 4246 | KTGLRHDSP | 4342 | KTINGSGSPH | 4438 |
| AQNCPR | | AQNQQT | | HKSGQKQQT | | SKAQRFGT | |
| KTINGSGSPHSK | 4151 | KTRLRSGSPHSK | 4247 | KTGLRHDSP | 4343 | KTINGSGSPH | 4439 |
| AQNMRR | | AQNQQT | | HKSGQNQQS | | SKAQRPCT | |
| KTINGSGSPHSK | 4152 | KWLLGSGSPHS | 4248 | KTGVTHDSP | 4344 | KTINGSGSPH | 4440 |
| AQNRRV | | KAQNQQT | | HKSGQKQQT | | SKAQRQAT | |
| KTINGSGSPHSK | 4153 | KWSQGSGSPHS | 4249 | KTIDGHESPH | 4345 | KTINGSGSPH | 4441 |
| AQPSRT | | KAQNQQT | | SKAQNQQT | | SKAQRQPT | |
| KTINGSGSPHSK | 4154 | KWYLGSGSPHS | 4250 | KTIEGHDSPH | 4346 | KTINGSGSPH | 4442 |
| AQQVKT | | KAQNQQT | | KSGQTQQT | | SKAQTKLT | |
| KTINGSGSPHSK | 4155 | KYHSGSGSPHS | 4251 | KTIHGHDSPH | 4347 | KTINGSGSPH | 4443 |
| AQQVRT | | KAQNQQT | | SKAQNQQT | | SKAQTTHT | |
| KTINGSGSPHSK | 4156 | KYLPGSGSPHSK | 4252 | KTIHGHESPH | 4348 | KTINGSGSPH | 4444 |
| AQRLKT | | AQNQQT | | SKAQNQQT | | SKAQVQRT | |
| KTINGSGSPHSK | 4157 | KAINGGGSPHSK | 4253 | KTIIGHDSPH | 4349 | KTINGSGSPH | 4445 |
| AQRRAT | | TQNQQT | | KSGQNRSS | | SKAQVVRT | |
| KTINGSGSPHSK | 4158 | KAINGHDSPHK | 4254 | KTIIGHDSPH | 4350 | KTINGSGSPH | 4446 |
| AQRRGT | | RSPNQQT | | KSGQRLGT | | SKAQWPNT | |
| KTINGSGSPHSK | 4159 | KAINGHDSPHKS | 4255 | KTIIGSGSPH | 4351 | KTINGSGSPH | 4447 |
| AQRRRT | | FSPQQT | | KSGQNQQT | | SKAQYPST | |
| KTINGSGSPHSK | 4160 | KAINGHDSPHKS | 4256 | KTIKGHDSPH | 4352 | KTINGSGSPH | 4448 |
| AQRTRT | | GENQQP | | KSGQNMLF | | SKARALQT | |
| KTINGSGSPHSK | 4161 | KAINGHDSPHKS | 4257 | KTILGSGSPH | 4353 | KTINGSGSPH | 4449 |
| AQRVHT | | GQLART | | SKAQNLQT | | SKARDQHT | |
| KTINGSGSPHSK | 4162 | KAINGHDSPHKS | 4258 | KTINGCSSPH | 4354 | KTINGSGSPH | 4450 |
| AQTYRT | | GQNAFL | | WKAQNQQT | | SKARFQQT | |
| KTINGSGSPHSK | 4163 | KAINGHDSPHKS | 4259 | KTINGGGSTH | 4355 | KTINGSGSPH | 4451 |
| AQVRKT | | GQNAYT | | SKAQNQQT | | SKARRTQT | |
| KTINGSGSPHSK | 4164 | KAINGHDSPHKS | 4260 | KTINGHDSPH | 4356 | KTINGSGSPH | 4452 |
| ARGRQT | | GQNFAS | | KAGQSQQT | | SKARSLQT | |
| KTINGSGSPHSK | 4165 | KAINGHDSPHKS | 4261 | KTINGHDSPH | 4357 | KTINGSGSPH | 4453 |
| ARLCQT | | GQNLAS | | KRGQNVPS | | SKARVIQT | |
| KTINGSGSPHSK | 4166 | KAINGHDSPHKS | 4262 | KTINGHDSPH | 4358 | KTINGSGSPH | 4454 |
| ARLKQT | | GQNLGS | | KRGRSYQT | | SKAWYLQT | |
| KTINGSGSPHSK | 4167 | KAINGHDSPHKS | 4263 | KTINGHDSPH | 4359 | KTINGSGSPH | 4455 |
| ARNSQT | | GQNLKF | | KTGQNPPT | | SKGGGQQT | |
| KTINGSGSPHSK | 4168 | KAINGHDSPHKS | 4264 | KTINGHDSPH | 4360 | KTINGSGSPH | 4456 |
| ARWVQT | | GQNLLK | | SKAENQQT | | SKGSRQQT | |
| KTINGSGSPHSK | 4169 | KAINGHDSPHKS | 4265 | KTINGHDSPH | 4361 | KTINGSGSPH | 4457 |
| AVRWQT | | GQNLSR | | SKALSLQT | | SKLQRQQT | |
| KTINGSGSPHSK | 4170 | KAINGHDSPHKS | 4266 | KTINGHDSPH | 4362 | KTINGSGSPH | 4458 |
| AYTRQT | | GQNLSS | | SKAQGQQT | | SKMLRQQT | |
| KTINGSGSPHSK | 4171 | KAINGHDSPHKS | 4267 | KTINGHDSPH | 4363 | KTINGSGSPH | 4459 |
| CQSQQT | | GQNSLG | | SKAQHQQT | | SKSSIKQT | |
| KTINGSGSPHSK | 4172 | KAINGHDSPHKS | 4268 | KTINGHDSPH | 4364 | KTINGSGSPH | 4460 |
| FLRQQT | | GQNTLQ | | SKAQIQQT | | SKVRFQQT | |
| KTINGSGSPHSK | 4173 | KAINGHDSPHKS | 4269 | KTINGHDSPH | 4365 | KTINGSGSPH | 4461 |
| FRFQQT | | GQNTSL | | SKAQKQQT | | SVVWNQQT | |
| KTINGSGSPHSK | 4174 | KAINGHDSPHKS | 4270 | KTINGHDSPH | 4366 | KTINGSTSPH | 4462 |
| FRLQQT | | GQRLGT | | SKAQNLSS | | KLAQNQQP | |
| KTINGSGSPHSK | 4175 | KAINGHDSPHKS | 4271 | KTINGHDSPH | 4367 | KTINRHDSPH | 4463 |
| FRRQQT | | GQRNYT | | SKAQNPQT | | KSGQRPST | |
| KTINGSGSPHSK | 4176 | KAINGHDSPHKS | 4272 | KTINGHDSPH | 4368 | KTINRIMSPH | 4464 |
| GMKQQT | | GQRPST | | SKAQNQET | | SKAQNQQT | |
| KTINGSGSPHSK | 4177 | KAINGHDSPHKS | 4273 | KTINGHDSPH | 4369 | KTINTARSPH | 4465 |
| KLRQQT | | GQRPVT | | SKAQNQHT | | SKAQNQQT | |
| KTINGSGSPHSK | 4178 | KAINGHDSPHKS | 4274 | KTINGHDSPH | 4370 | KTISGHDSPH | 4466 |
| KRPQQT | | GQVPST | | SKAQNQLT | | SKAQNQQT | |
| KTINGSGSPHSK | 4179 | KAINGHDSPHKS | 4275 | KTINGHDSPH | 4371 | KTISGSGSPH | 4467 |
| KSRQQT | | LSNQQT | | SKAQNQPT | | KSGQNQQT | |
| KTINGSGSPHSK | 4180 | KAINGHDSPHKS | 4276 | KTINGHDSPH | 4372 | KTITGHDSPH | 4468 |
| LYRQQT | | VLSQQT | | SKAQNQQA | | KSGQRLGT | |
| KTINGSGSPHSK | 4181 | KAINGHDSPHKT | 4277 | KTINGHDSPH | 4373 | KTITGSGSPH | 4469 |
| LYWQQT | | LQNQQT | | SKAQNTGS | | KSGQNQQT | |
| KTINGSGSPHSK | 4182 | KAINGHNSPHSK | 4278 | KTINGHDSPH | 4374 | KTIYGHDSP | 4470 |
| PRMQQT | | AQNQQT | | SKAQSQQT | | HKSGQRLGT | |
| KTINGSGSPHSK | 4183 | KAINGLDSPHSK | 4279 | KTINGHDSPH | 4375 | KTLNGHDSP | 4471 |
| RFPQQT | | AQNQQT | | SKAQTQQT | | HKSGQNLFL | |
| KTINGSGSPHSK | 4184 | KAINGSGSPHKS | 4280 | KTINGHDSPH | 4376 | KTLNGHDSP | 4472 |
| RFRQQT | | GQNQQT | | SKAQYQQT | | HKSGQNLSS | |
| KTINGSGSPHSK | 4185 | KAINGSGSPHSK | 4281 | KTINGHDSPH | 4377 | KTLSFHDSPH | 4473 |
| RPYQQT | | AQGQQT | | SKARNQQT | | KSGQNQQS | |
| KTINGSGSPHSK | 4186 | KAINGSGSPHSK | 4282 | KTINGHDSPH | 4378 | KTSNGSGSP | 4474 |
| RRMQQT | | AQLSGT | | SKLPGQQT | | HSKAQNTMT | |
| KTINGSGSPHSK | 4187 | KAINGSGSPHSK | 4283 | KTINGHDSPH | 4379 | KTTNGHDSP | 4475 |
| RSKQQT | | AQNGSL | | SKSPNQQT | | HSKAQNQQT | |
| KTINGSGSPHSK | 4188 | KAINGSGSPHSK | 4284 | KTINGHESPH | 4380 | KTVNGGGSP | 4476 |
| RSRQQT | | AQNSLL | | KSGQNAFL | | HSKAQNQQT | |
| KTINGSGSPHSK | 4189 | KAINGSGSPHSK | 4285 | KTINGIGSPH | 4381 | KTVNGHDSP | 4477 |
| RTMQQT | | AVGLQT | | SKAPNEQT | | HKSGQNVSL | |
| KTINGSGSPHSK | 4190 | KAINGSGSPHSK | 4286 | KTINGQDSPH | 4382 | KTVNGHDSP | 4478 |
| RTRQQT | | SLLQQT | | KSGQNLHM | | HKSGQRPST | |
| KTINGSGSPHSK | 4191 | KAINGSGSPHSK | 4287 | KTINGRGSPH | 4383 | KTVNGHDSP | 4479 |
| RVRQQT | | SLPQQT | | SKAQIGMT | | HKSGQTQQA | |
| KTINGSGSPHSK | 4192 | KAINGSGSPHSK | 4288 | KTINGRGSPH | 4384 | KTVNGHESP | 4480 |
| RYIQQT | | STFQQT | | SKAQNQVL | | HSKAQNQQT | |
| KTINGSGSPHSK | 4193 | KAITGHDSPHSK | 4289 | KTINGRGSPH | 4385 | KTVNGSGSP | 4481 |
| RYNQQT | | AQNQQT | | SKAQSPTT | | HSKAQGLST | |
| KTINGSGSPHSK | 4194 | KDVMGSGSPHS | 4290 | KTINGRGSPH | 4386 | KTVNGSGSP | 4482 |
| RYPQQT | | KAQNQQT | | SKATSFQT | | HSKAQNVTS | |
| KTINGSGSPHSK | 4195 | KEIVGSGSPHSK | 4291 | KTINGSGSPH | 4387 | KTVPASGSP | 4483 |
| RYSQQT | | AQNQQT | | FVVQNQQT | | HSKAQNQQT | |
| NTINGSGSPHSK | 4484 | TTINGGGSPHSK | 4485 | KTINGSGSPH | 4081 | | |
| AHNQQT | | AQNQQT | | KSGQNQQP | | | |

TABLE 2A
Exemplary Peptide Sequences

| SEQ ID | Amino Acid | SEQ ID | |
|---|---|---|---|
| NO: | Sequence | NO: | Nucleotide Sequence |
| 941 | SPHSKA | 942 | AGCCCACACAGCAAAGCA |
| 943 | HDSPHKSG | 944 | CACGACAGCCCACACAAAA |
| | | | GCGGA |
| 2 | HDSPHK | 3 | CACGACAGCCCACACAAA |

TABLE 2B
Exemplary Peptide Sequences

| Amino Acid | SEQ ID | Amino Acid | SEQ ID | Amino Acid | SEQ ID | Amino Acid | SEQ ID |
|---|---|---|---|---|---|---|---|
| Sequence | NO: | Sequence | NO: | Sequence | NO: | Sequence | NO: |
| SPHSKA | 945 | SPHKKN | 954 | SPHKTS | 963 | SPHTRG | 972 |
| SPHKSG | 946 | SPHVRM | 955 | SPHKTT | 964 | SPHVRG | 973 |
| SPHARM | 947 | SPHRKA | 956 | SPHKTY | 965 | SPHKRG | 974 |
| SPHVKS | 948 | SPHKFG | 957 | SPHKYG | 966 | SPHGAR | 975 |
| SPHASR | 949 | SPHKIG | 958 | SPHSKD | 967 | SPHRSG | 976 |
| SPHVKI | 950 | SPHKLG | 959 | SPHSKP | 968 | SPHKSA | 977 |
| SPHKSR | 951 | SPHSKL | 960 | SPHSRA | 969 | SPHSKR | 978 |
| SPHSLR | 952 | SPHSRG | 961 | SPHSSR | 970 | SPHFLR | 979 |
| SPHSKW | 953 | SPHSKS | 962 | SPHWKA | 971 | SPHVRR | 980 |
| STHASR | 985 | SQHKSG | 986 | HDSPHK | 2 | HDSPHSKA | 4486 |

In some embodiments, an AAV capsid variant described herein comprises an amino acid sequence having the formula [N1]-[N2]-[N3], wherein [N2] comprises the amino acid sequence of SPH and [N3] comprises X4, X5, and X6, wherein at least one of X4, X5, or X6 is a basic amino acid, e.g., a K or R. In some embodiments, position X4 of [N2] is K. In some embodiments, position X5 of [N2] is K.

In some embodiments, [N1] comprises X1, X2, and X3, wherein at least one of X1, X2, or X3 is G. In some embodiments, position X1 of [N1] is: G, V, R, D, E, M, T, I, S, A, N, L, K, H, P, W, or C. In some embodiments, position X2 of [N1] is: S, V, L, N, D, H, R, P, G, T, I, A, E, Y, M, or Q. In some embodiments, position X3 of [N1] is: G, C, L, D, E, Y, H, V, A, N, P, or S. In some embodiments, [N1] comprises GS, SG, GH, HD, GQ, QD, VS, CS, GR, RG, QS, SH, MS, RN, TS, IS, GP, ES, SS, GN, AS, NS, LS, GG, KS, GT, PS, RS, GI, WS, DS, ID, GL, DA, DG, ME, EN, KN, KE, AI, NG, PG, TG, SV, IG, LG, AG, EG, SA, YD, HE, HG, RD, ND, PD, MG, QV, DD, HN, HP, GY, GM, GD, or HS. In some embodiments, [N1] comprises GS, SG, GH, or HD. In some embodiments [N1] is or comprises GSG, GHD, GQD, VSG, CSG, CSH, GQS, GRG, GSH, RVG, GSC, GLL, GDD, GHE, GNY, MSG, RNG, TSG, ISG, GPG, ESG, SSG, GNG, ASG, NSG, LSG, GGG, KSG, HSG, GTG, PSG, GSV, RSG, GIG, WSG, DSG, IDG, GLG, DAG, DGG, MEG, ENG, GSA, KNG, KEG, AIG, GYD, GHG, GRD, GND, GPD, GMG, GQV, GHN, GHP, or GHS. In some embodiments, [N1] is or comprises GSG. In some embodiments, [N1] is or comprises GHD. In some embodiments, [N1]-[N2] comprises SGSPH (SEQ ID NO: 1424), HDSPH (SEQ ID NO: 1374), QDSPH (SEQ ID NO: 1425), RGSPH (SEQ ID NO: 1426), SHSPH (SEQ ID NO: 1427), QSSPH (SEQ ID NO: 1428), DDSPH (SEQ ID NO: 1429), HESPH (SEQ ID NO: 1430), NYSPH (SEQ ID NO: 1431), VGSPH (SEQ ID NO: 1432), SCSPH (SEQ ID NO: 1433), LLSPH (SEQ ID NO: 1434), NGSPH (SEQ ID NO: 1435), PGSPH (SEQ ID NO: 1436), GGSPH (SEQ ID NO: 1437), TGSPH (SEQ ID NO: 1438), SVSPH (SEQ ID NO: 1439), IGSPH (SEQ ID NO: 1440), DGSPH (SEQ ID NO: 1441), LGSPH (SEQ ID NO: 1442), AGSPH (SEQ ID NO: 1443), EGSPH (SEQ ID NO: 1444), SASPH (SEQ ID NO: 1445), YDSPH (SEQ ID NO: 1446), HGSPH (SEQ ID NO: 1447), RDSPH (SEQ ID NO: 1448), NDSPH (SEQ ID NO: 1449), PDSPH (SEQ ID NO: 1450), MGSPH (SEQ ID NO: 1451), QVSPH (SEQ ID NO: 1452), HNSPH (SEQ ID NO: 1453), HPSPH (SEQ ID NO: 1454), or HSSPH (SEQ ID NO: 1455); an amino acid sequence comprising any portion of any of the aforesaid amino acid sequences (e.g., any 2, 3, or 4 amino acids, e.g., consecutive amino acids) thereof; an amino acid sequence comprising one, two, or three but no more than four modifications, e.g., substitutions (e.g., conservative substitutions), insertions, or deletions, relative to any of the aforesaid amino acid sequences; or an amino acid sequence comprising one, two, or three but no more than four different amino acids, relative to any one of the aforesaid amino acid sequences. In some embodiments, [N1]-[N2] is or comprises GSGSPH (SEQ ID NO: 1456), GHDSPH (SEQ ID NO: 1457), GQDSPH (SEQ ID NO: 1458), VSGSPH (SEQ ID NO: 1459), CSGSPH (SEQ ID NO: 1460), GRGSPH (SEQ ID NO: 1461), CSHSPH (SEQ ID NO: 1462), GQSSPH (SEQ ID NO: 1463), GSHSPH (SEQ ID NO: 1464), GDDSPH (SEQ ID NO: 1465), GHESPH (SEQ ID NO: 1466), GNYSPH (SEQ ID NO: 1467), RVGSPH (SEQ ID NO: 1468), GSCSPH (SEQ ID NO: 1469), GLLSPH (SEQ ID NO: 1470), MSGSPH (SEQ ID NO: 1471), RNGSPH (SEQ ID NO: 1472), TSGSPH (SEQ ID NO: 1473), ISGSPH (SEQ ID NO: 1474), GPGSPH (SEQ ID NO: 1475), ESGSPH (SEQ ID NO: 1476), SSGSPH (SEQ ID NO: 1477), GNGSPH (SEQ ID NO: 1478), ASGSPH (SEQ ID NO: 1479), NSGSPH (SEQ ID NO: 1480), LSGSPH (SEQ ID NO: 1481), GGGSPH (SEQ ID NO: 1482), KSGSPH (SEQ ID NO: 1483), HSGSPH (SEQ ID NO: 1484), GTGSPH (SEQ ID NO: 1485), PSGSPH (SEQ ID NO: 1486), GSVSPH (SEQ ID NO: 1487), RSGSPH (SEQ ID NO: 1488), GIGSPH (SEQ ID NO: 1489), WSGSPH (SEQ ID NO: 1490), DSGSPH (SEQ ID NO: 1491), IDGSPH (SEQ ID NO: 1492), GLGSPH (SEQ ID NO: 1493), DAGSPH (SEQ ID NO: 1494), DGGSPH (SEQ ID NO: 1495), MEGSPH (SEQ ID NO: 1496), ENGSPH (SEQ ID NO: 1497), GSASPH (SEQ ID NO: 1498), KNGSPH (SEQ ID NO: 1499), KEGSPH (SEQ ID NO: 1500), AIGSPH (SEQ ID NO: 1501), GYDSPH (SEQ ID NO: 1502), GHGSPH (SEQ ID NO: 1503), GRDSPH (SEQ ID NO: 1504), GNDSPH (SEQ ID NO: 1505), GPDSPH (SEQ ID NO: 1506), GMGSPH (SEQ ID NO: 1507), GQVSPH (SEQ ID NO: 1508), GHNSPH (SEQ ID NO: 1509), GHPSPH (SEQ ID NO: 1510), or GHSSPH (SEQ ID NO: 1511); an amino acid sequence comprising any portion of any of the aforesaid amino acid sequences (e.g., any 2, 3, 4, or 5 amino acids, e.g., consecutive amino acids) thereof; an amino acid sequence comprising one, two, or three but no more than four modifications, e.g., substitutions (e.g., conservative substitutions), insertions, or deletions, relative to any of the aforesaid amino acid sequences; or an amino acid sequence comprising one, two, or three but no more than four different amino acids, relative to any one of the aforesaid amino acid sequences. In some embodiments, [N1]-[N2] is or comprises GSGSPH (SEQ ID NO: 1456). In some embodiments, [N1]-[N2] is or comprises GHDSPH (SEQ ID NO: 1457).

In some embodiments, X4, X5, or both of [N3] are K. In some embodiments, X4, X5, or X6 of [N3] is R. In some embodiments, position X4 of [N3] is: A, K, V, S, T, G, F, W, V, N, or R. In some embodiments, position X5 of [N3] is: S, K, T, F, I, L, Y, H, M, or R. In some embodiments, position X6 of [N3] is: G, R, A, M, I, N, T, Y, D, P, V, L, E, W, N, Q, K, or S. In some embodiments, [N3] comprises SK, KA, KS, AR, RM, VK, AS, SR, VK, KR, KK, KN, VR, RS, RK, KT, TS, KF, FG, KI, IG, KL, LG, TT, TY, KY, YG, KD, KP, TR, RG, VR, GA, SL, SS, FL, WK, SA, RA, LR, KW, RR, GK, TK, NK, AK, KV, KG, KH, KM, TG, SE, SV, SW, SN, HG, SQ, LW, MG, MA, or SG. In some embodiments, [N3] comprises SK, KA, KS, or SG. In some embodiments, [N3] is or comprises SKA, KSG, ARM, VKS, ASR, VKI, KKN, VRM, RKA, KTS, KFG, KIG, KLG, KTT, KTY, KYG, SKD, SKP, TRG, VRG, KRG, GAR, KSA, KSR, SKL, SRA, SKR, SLR, SRG, SSR, FLR, SKW, SKS, WKA, VRR, SKV, SKT, SKG, GKA, TKA, NKA, SKL, SKN, AKA, KTG, KSL, KSE, KSV, KSW, KSN, KHG, KSQ, KSK, KLW, WKG, KMG, KMA, or RSG. In some embodiments, [N3] is or comprises SKA. In some embodiments, [N3] is or comprises KSG. In some embodiments, [N2]-[N3] comprises SPHSK (SEQ ID NO: 1372), SPHKS (SEQ ID NO: 1375), SPHAR (SEQ ID NO: 1376), SPHVK (SEQ ID NO: 1377), SPHAS (SEQ ID NO: 1378), SPHKK (SEQ ID NO: 1379), SPHVR (SEQ ID NO: 1380), SPHRK (SEQ ID NO: 1381), SPHKT (SEQ ID NO: 1382), SPHKF (SEQ ID NO: 1383), SPHKI (SEQ ID NO: 1384), SPHKL (SEQ ID NO: 1385), SPHKY (SEQ ID NO: 1386), SPHTR (SEQ ID NO: 1387), SPHKR (SEQ ID NO: 1388), SPHGA (SEQ ID NO: 1389), SPHSR (SEQ ID NO: 1390), SPHSL (SEQ ID NO: 1391), SPHSS (SEQ ID NO: 1392), SPHFL (SEQ ID NO: 1393), SPHWK (SEQ ID NO: 1394), SPHGK (SEQ ID NO: 1395), SPHTK (SEQ ID NO: 1396), SPHNK (SEQ ID NO: 1397), SPHAK (SEQ ID NO: 1398), SPHKH (SEQ ID NO: 1399), SPHKM (SEQ ID NO: 1400), or SPHRS (SEQ ID NO: 1401). In some embodiments [N2]-[N3] comprises SPHSK (SEQ ID NO: 1372) or SPHKS (SEQ ID NO: 1375). In some embodiments, [N2]-[N3] is or comprises SPHSKA (SEQ ID NO: 941), SPHKSG (SEQ ID NO: 946), SPHARM (SEQ ID NO: 947), SPHVKS (SEQ ID NO: 948), SPHASR (SEQ ID NO: 949), SPHVKI (SEQ ID NO: 950), SPHKKN (SEQ ID NO: 954), SPHVRM (SEQ ID NO: 955), SPHRKA (SEQ ID NO: 956), SPHKFG (SEQ ID NO: 957), SPHKIG (SEQ ID NO: 958), SPHKLG (SEQ ID NO: 959), SPHKTS (SEQ ID NO: 963), SPHKTT (SEQ ID NO: 964), SPHKTY (SEQ ID NO: 965), SPHKYG (SEQ ID NO: 966), SPHSKD (SEQ ID NO: 967), SPHSKP (SEQ ID NO: 968), SPHTRG (SEQ ID NO: 972), SPHVRG (SEQ ID NO: 973), SPHKRG (SEQ ID NO: 974), SPHGAR (SEQ ID NO: 975), SPHKSA (SEQ ID NO: 977), SPHKSR (SEQ ID NO: 951), SPHSKL (SEQ ID NO: 960), SPHSRA (SEQ ID NO: 969), SPHSKR (SEQ ID NO: 978), SPHSLR (SEQ ID NO: 952), SPHSRG (SEQ ID NO: 961), SPHSSR (SEQ ID NO: 970), SPHFLR (SEQ ID NO: 979), SPHSKW (SEQ ID NO: 953), SPHSKS (SEQ ID NO: 962), SPHWKA (SEQ ID NO: 971), SPHVRR (SEQ ID NO: 980), SPHSKT (SEQ ID NO: 1403), SPHSKG (SEQ ID NO: 1404), SPHGKA (SEQ ID NO: 1405), SPHNKA (SEQ ID NO: 1406), SPHSKN (SEQ ID NO: 1407), SPHAKA (SEQ ID NO: 1408), SPHSKV (SEQ ID NO: 1409), SPHKTG (SEQ ID NO: 1410), SPHTKA (SEQ ID NO: 1411), SPHKSL (SEQ ID NO: 1412), SPHKSE (SEQ ID NO: 1413), SPHKSV (SEQ ID NO: 1414), SPHKSW (SEQ ID NO: 1415), SPHKSN (SEQ ID NO: 1416), SPHKHG (SEQ ID NO: 1417), SPHKSQ (SEQ ID NO: 1418), SPHKSK (SEQ ID NO: 1419), SPHKLW (SEQ ID NO: 1420), SPHWKG (SEQ ID NO: 1421), SPHKMG (SEQ ID NO: 1422), SPHKMA (SEQ ID NO: 1423), or SPHRSG (SEQ ID NO: 976). In some embodiments, [N2]-[N3] is SPHSKA (SEQ ID NO: 941). In some embodiments, [N2]-[N3] is or comprises SPHKSG (SEQ ID NO: 946).

In some embodiments, [N1]-[N2]-[N3] comprises SGSPHSK (SEQ ID NO: 1512), HDSPHKS (SEQ ID NO: 1513), SGSPHAR (SEQ ID NO: 1514), SGSPHVK (SEQ ID NO: 1515), QDSPHKS (SEQ ID NO: 1516), SGSPHKK (SEQ ID NO: 1517), SGSPHVR (SEQ ID NO: 1518), SGSPHAS (SEQ ID NO: 1519), SGSPHRK (SEQ ID NO: 1520), SGSPHKT (SEQ ID NO: 1521), SHSPHKS (SEQ ID NO: 1522), QSSPHRS (SEQ ID NO: 1523), RGSPHAS (SEQ ID NO: 1524), RGSPHSK (SEQ ID NO: 1525), SGSPHKF (SEQ ID NO: 1526), SGSPHKI (SEQ ID NO: 1527), SGSPHKL (SEQ ID NO: 1528), SGSPHKY (SEQ ID NO: 1529), SGSPHTR (SEQ ID NO: 1530), SHSPHKR (SEQ ID NO: 1531), SGSPHGA (SEQ ID NO: 1532), HDSPHKR (SEQ ID NO: 1533), DDSPHKS (SEQ ID NO: 1534), HESPHKS (SEQ ID NO: 1535), NYSPHKI (SEQ ID NO: 1536), SGSPHSR (SEQ ID NO: 1537), SGSPHSL (SEQ ID NO: 1538), SGSPHSS (SEQ ID NO: 1539), VGSPHSK (SEQ ID NO: 1540), SCSPHRK (SEQ ID NO: 1541), SGSPHFL (SEQ ID NO: 1542), LLSPHWK (SEQ ID NO: 1543), NGSPHSK (SEQ ID NO: 1544), PGSPHSK (SEQ ID NO: 1545), GGSPHSK (SEQ ID NO: 1546), TGSPHSK (SEQ ID NO: 1547), SVSPHGK (SEQ ID NO: 1548), SGSPHTK (SEQ ID NO: 1549), IGSPHSK (SEQ ID NO: 1550), DGSPHSK (SEQ ID NO: 1551), SGSPHNK (SEQ ID NO: 1552), LGSPHSK (SEQ ID NO: 1553), AGSPHSK (SEQ ID NO: 1554), EGSPHSK (SEQ ID NO: 1555), SASPHSK (SEQ ID NO: 1556), SGSPHAK (SEQ ID NO: 1557), HDSPHKI (SEQ ID NO: 1558), YDSPHKS (SEQ ID NO: 1559), HDSPHKT (SEQ ID NO: 1560), RGSPHKR (SEQ ID NO: 1561), HGSPHSK (SEQ ID NO: 1562), RDSPHKS (SEQ ID NO: 1563), NDSPHKS (SEQ ID NO: 1564), QDSPHKI (SEQ ID NO: 1565), PDSPHKI (SEQ ID NO: 1566), PDSPHKS (SEQ ID NO: 1567), MGSPHSK (SEQ ID NO: 1568), HDSPHKH (SEQ ID NO: 1569), QVSPHKS (SEQ ID NO: 1570), HNSPHKS (SEQ ID NO: 1571), NGSPHKR (SEQ ID NO: 1572), HDSPHKY (SEQ ID NO: 1573), NDSPHKI (SEQ ID NO: 1574), HDSPHKL (SEQ ID NO: 1575), HPSPHWK (SEQ ID NO: 1576), HDSPHKM (SEQ ID NO: 1577), or HSSPHRS (SEQ ID NO: 1578). In some embodiments, [N1]-[N2]-[N3] is or comprises GSGSPHSKA (SEQ ID NO: 1369), GHDSPHKSG (SEQ ID NO: 1370), GSGSPHARM (SEQ ID NO: 1579), GSGSPHVKS (SEQ ID NO: 1580), GQDSPHKSG (SEQ ID NO: 1581), GSGSPHASR (SEQ ID NO: 1582), GSGSPHVKI (SEQ ID NO: 1583), GSGSPHKKN (SEQ ID NO: 1584), GSGSPHVRM (SEQ ID NO: 1585), VSGSPHSKA (SEQ ID NO: 1586), CSGSPHSKA (SEQ ID NO: 1587), GSGSPHRKA (SEQ ID NO: 1588), CSGSPHKTS (SEQ ID NO: 1589), CSHSPHKSG (SEQ ID NO: 1590), GQSSPHRSG (SEQ ID NO: 1591), GRGSPHASR (SEQ ID NO: 1592), GRGSPHSKA (SEQ ID NO: 1593), GSGSPHKFG (SEQ ID NO: 1594), GSGSPHKIG (SEQ ID NO: 1595), GSGSPHKLG (SEQ ID NO: 1596), GSGSPHKTS (SEQ ID NO: 1597), GSGSPHKTT (SEQ ID NO: 1598), GSGSPHKTY (SEQ ID NO: 1599), GSGSPHKYG (SEQ ID NO: 1600), GSGSPHSKD (SEQ ID NO: 1601), GSGSPHSKP (SEQ ID NO: 1602), GSGSPHTRG (SEQ ID NO: 1603), GSGSPHVRG (SEQ ID NO: 1604), GSHSPHKRG (SEQ ID NO: 1605), GSHSPHKSG (SEQ ID NO: 1606), VSGSPHASR (SEQ ID NO: 1607), VSGSPHGAR (SEQ ID NO: 1608), VSGSPHKFG (SEQ ID NO: 1609), GHDSPHKRG (SEQ ID NO: 1610), GDDSPHKSG (SEQ ID NO: 1611), GHESPHKSA (SEQ ID NO: 1612), GHDSPHKSA (SEQ ID NO: 1613), GNYSPHKIG (SEQ ID NO: 1614), GHDSPHKSR (SEQ ID NO: 1615), GSGSPHSKL (SEQ ID NO: 1616), GSGSPHSRA (SEQ ID NO: 1617), GSGSPHSKR (SEQ ID NO: 1618), GSGSPHSLR (SEQ ID NO: 1619), GSGSPHSRG (SEQ ID NO: 1620), GSGSPHSSR (SEQ ID NO: 1621), RVGSPHSKA (SEQ ID NO: 1622), GSCSPHRKA (SEQ ID NO: 1623), GSGSPHFLR (SEQ ID NO: 1624), GSGSPHSKW (SEQ ID NO: 1625), GSGSPHSKS (SEQ ID NO: 1626), GLLSPHWKA (SEQ ID NO: 1627), GSGSPHVRR (SEQ ID NO: 1628), GSGSPHSKV (SEQ ID NO: 1629), MSGSPHSKA (SEQ ID NO: 1630), RNGSPHSKA (SEQ ID NO: 1631), TSGSPHSKA (SEQ ID NO: 1632), ISGSPHSKA (SEQ ID NO: 1633), GPGSPHSKA (SEQ ID NO: 1634), GSGSPHSKT (SEQ ID NO: 1635), ESGSPHSKA (SEQ ID NO: 1636), SSGSPHSKA (SEQ ID NO: 1637), GNGSPHSKA (SEQ ID NO: 1638), ASGSPHSKA (SEQ ID NO: 1639), NSGSPHSKA (SEQ ID NO: 1640), LSGSPHSKA (SEQ ID NO: 1641), GGGSPHSKA (SEQ ID NO: 1642), KSGSPHSKA (SEQ ID NO: 1643), GGGSPHSKS (SEQ ID NO: 1644), GSGSPHSKG (SEQ ID NO: 1645), HSGSPHSKA (SEQ ID NO: 1646), GTGSPHSKA (SEQ ID NO: 1647), PSGSPHSKA (SEQ ID NO: 1648), GSVSPHGKA (SEQ ID NO: 1649), RSGSPHSKA (SEQ ID NO: 1650), GSGSPHTKA (SEQ ID NO: 1651), GIGSPHSKA (SEQ ID NO: 1652), WSGSPHSKA (SEQ ID NO: 1653), DSGSPHSKA (SEQ ID NO: 1654), IDGSPHSKA (SEQ ID NO: 1655), GSGSPHNKA (SEQ ID NO: 1656), GLGSPHSKS (SEQ ID NO: 1657), DAGSPHSKA (SEQ ID NO: 1658), DGGSPHSKA (SEQ ID NO: 1659), MEGSPHSKA (SEQ ID NO: 1660), ENGSPHSKA (SEQ ID NO: 1661), GSASPHSKA (SEQ ID NO: 1662), GNGSPHSKS (SEQ ID NO: 1663), KNGSPHSKA (SEQ ID NO: 1664), KEGSPHSKA (SEQ ID NO: 1665), AIGSPHSKA (SEQ ID NO: 1666), GSGSPHSKN (SEQ ID NO: 1667), GSGSPHAKA (SEQ ID NO: 1668), GHDSPHKIG (SEQ ID NO: 1669), GYDSPHKSG (SEQ ID NO: 1670), GHESPHKSG (SEQ ID NO: 1671), GHDSPHKTG (SEQ ID NO: 1672), GRGSPHKRG (SEQ ID NO: 1673), GQDSPHKSG (SEQ ID NO: 1581), GHDSPHKSL (SEQ ID NO: 1674), GHGSPHSKA (SEQ ID NO: 1675), GHDSPHKSE (SEQ ID NO: 1676), VSGSPHSKA (SEQ ID NO: 1586), GRDSPHKSG (SEQ ID NO: 1677), GNDSPHKSV (SEQ ID NO: 1678), GQDSPHKIG (SEQ ID NO: 1679), GHDSPHKSV (SEQ ID NO: 1680), GPDSPHKIG (SEQ ID NO: 1681), GPDSPHKSG (SEQ ID NO: 1682), GHDSPHKSW (SEQ ID NO: 1683), GHDSPHKSN (SEQ ID NO: 1684), GMGSPHSKT (SEQ ID NO: 1685), GHDSPHKHG (SEQ ID NO: 1686), GQVSPHKSG (SEQ ID NO: 1687), GDDSPHKSV (SEQ ID NO: 1688), GHNSPHKSG (SEQ ID NO: 1689), GNGSPHKRG (SEQ ID NO: 1690), GHDSPHKYG (SEQ ID NO: 1691), GHDSPHKSQ (SEQ ID NO: 1692), GNDSPHKIG (SEQ ID NO: 1693), GHDSPHKSK (SEQ ID NO: 1694), GHDSPHKLW (SEQ ID NO: 1695), GHPSPHWKG (SEQ ID NO: 1696), GHDSPHKMG (SEQ ID NO: 1697), GHDSPHKMA (SEQ ID NO: 1698), or GHSSPHRSG (SEQ ID NO: 1699); an amino acid sequence comprising any portion of any of the aforesaid amino acid sequences (e.g., any 2, 3, 4, 5, 6, 7, or 8 amino acids, e.g., consecutive amino acids) thereof; an amino acid sequence comprising one, two, or three but no more than four modifications, e.g., substitutions (e.g., conservative substitutions), insertions, or deletions, relative to any of the aforesaid amino acid sequences; or an amino acid sequence comprising one, two, or three but no more than four different amino acids, relative to any one of the aforesaid amino acid sequences. In some embodiments, [N1]-[N2]-[N3] is or comprises GSGSPHSKA (SEQ ID NO: 1369). In some embodiments, [N1]-[N2]-[N3] is or comprises GHDSPHKSG (SEQ ID NO: 1370).

In some embodiments, the AAV capsid variant comprising an amino acid sequence having the formula [N1]-[N2]-[N3], further comprises [N4], wherein [N4] comprises X7 X8 X9 X10. In some embodiments, position X7 of [N4] is: W, Q, K, R, G, L, V, S, P, H, K, I, M, A, E, or F. In some embodiments, position X8 of [N4] is: N, Y, C, K, T, H, R, D, V, S, P, G, W, E, F, A, I, M, Q, or L. In some embodiments, position X9 of [N4] is: Q, G, K, H, R, T, L, D, A, P, I, F, V, M, W, Y, S, E, N, or Y. In some embodiments, position X10 of [N4] is: Q, H, L, R, W, K, A, P, E, M, I, S, G, N, Y, C, V, T, D, or V. In some embodiments [N4] comprises QNQQ (SEQ ID NO: 1701), WNQQ (SEQ ID NO: 1702), QYYV (SEQ ID NO: 1703), RRQQ (SEQ ID NO: 1704), GCGQ (SEQ ID NO: 1705), LRQQ (SEQ ID NO: 1706), RNQQ (SEQ ID NO: 1707), VNQQ (SEQ ID NO: 1708), FRLQ (SEQ ID NO: 1709), FNQQ (SEQ ID NO: 1710), LLQQ (SEQ ID NO: 1711), SNQQ (SEQ ID NO: 1712), RLQQ (SEQ ID NO: 1713), LNQQ (SEQ ID NO: 1714), QRKL (SEQ ID NO: 1715), LRRQ (SEQ ID NO: 1716), QRLR (SEQ ID NO: 1717), QRRL (SEQ ID NO: 1718), RRLQ (SEQ ID NO: 1719), RLRQ (SEQ ID NO: 1720), SKRQ (SEQ ID NO: 1721), QLYR (SEQ ID NO: 1722), QLTV (SEQ ID NO: 1723), QNKQ (SEQ ID NO: 1724), KNQQ (SEQ ID NO: 1725), QKQQ (SEQ ID NO: 1726), QTQQ (SEQ ID NO: 1727), QNHQ (SEQ ID NO: 1728), QHQQ (SEQ ID NO: 1729), QNQH (SEQ ID NO: 1730), QHRQ (SEQ ID NO: 1731), LTQQ (SEQ ID NO: 1732), QNQW (SEQ ID NO: 1733), QNTH (SEQ ID NO: 1734), RRRQ (SEQ ID NO: 1735), QYQQ (SEQ ID NO: 1736), QNDQ (SEQ ID NO: 1737), QNRH (SEQ ID NO: 1738), RDQQ (SEQ ID NO: 1739), PNLQ (SEQ ID NO: 1740), HVRQ (SEQ ID NO: 1741), PNQH (SEQ ID NO: 1742), HNQQ (SEQ ID NO: 1743), QSQQ (SEQ ID NO: 1744), QPAK (SEQ ID NO: 1745), QNLA (SEQ ID NO: 1746), QNQL (SEQ ID NO: 1747), QGQQ (SEQ ID NO: 1748), LNRQ (SEQ ID NO: 1749), QNPP (SEQ ID NO: 1750), QNLQ (SEQ ID NO: 1751), QDQE (SEQ ID NO: 1752), QDQQ (SEQ ID NO: 1753), HWQQ (SEQ ID NO: 1755), PNQQ (SEQ ID NO: 1756), PEQQ (SEQ ID NO: 1757), QRTM (SEQ ID NO: 1758), LHQH (SEQ ID NO: 1759), QHRI (SEQ ID NO: 1760), QYIH (SEQ ID NO: 1761), QKFE (SEQ ID NO: 1762), QFPS (SEQ ID NO: 1763), QNPL (SEQ ID NO: 1764), QAIK (SEQ ID NO: 1765), QNRQ (SEQ ID NO: 1766), QYQH (SEQ ID NO: 1767), QNPQ (SEQ ID NO: 1768), QHQL (SEQ ID NO: 1769), QSPP (SEQ ID NO: 1770), QAKL (SEQ ID NO: 1771), KSQQ (SEQ ID NO: 1772), QDRP (SEQ ID NO: 1773), QNLG (SEQ ID NO: 1774), QAFH (SEQ ID NO: 1775), QNAQ (SEQ ID NO: 1776), HNQL (SEQ ID NO: 1777), QKLN (SEQ ID NO: 1778), QNVQ (SEQ ID NO: 1779), QAQQ (SEQ ID NO: 1780), QTPP (SEQ ID NO: 1781), QPPA (SEQ ID NO: 1782), QERP (SEQ ID NO: 1783), QDLQ (SEQ ID NO: 1784), QAMH (SEQ ID NO: 1785), QHPS (SEQ ID NO: 1786), PGLQ (SEQ ID NO: 1787), QGIR (SEQ ID NO: 1788), QAPA (SEQ ID NO: 1789), QIPP (SEQ ID NO: 1790), QTQL (SEQ ID NO: 1791), QAPS (SEQ ID NO: 1792), QNTY (SEQ ID NO: 1793), QDKQ (SEQ ID NO: 1794), QNHL (SEQ ID NO: 1795), QIGM (SEQ ID NO: 1796), LNKQ (SEQ ID NO: 1797), PNQL (SEQ ID NO: 1798), QLQQ (SEQ ID NO: 1799), QRMS (SEQ ID NO: 2897), QGIL (SEQ ID NO: 2907), QDRQ (SEQ ID NO: 2917), RDWQ (SEQ ID NO: 3081), QERS (SEQ ID NO: 3238), QNYQ (SEQ ID NO: 3590), QRTC (SEQ ID NO: 3849), QIGH (SEQ ID NO: 3850), QGAI (SEQ ID NO: 3851), QVPP (SEQ ID NO: 3852), QVQQ (SEQ ID NO: 3853), LMRQ (SEQ ID NO: 3854), QYSV (SEQ ID NO: 3855), QAIT (SEQ ID NO: 3856), QKTL (SEQ ID NO: 3857), QLHH (SEQ ID NO: 3858), QNII (SEQ ID NO: 3859), QGHH (SEQ ID NO: 3860), QSKV (SEQ ID NO: 3861), QLPS (SEQ ID NO: 3862), IGKQ (SEQ ID NO: 3863), QAIH (SEQ ID NO: 3864), QHGL (SEQ ID NO: 3865), QFMC (SEQ ID NO: 3866), QNQM (SEQ ID NO: 3867), QHLQ (SEQ ID NO: 3868), QPAR (SEQ ID NO: 3869), QSLQ (SEQ ID NO: 3870), QSQL (SEQ ID NO: 3871), HSQQ (SEQ ID NO: 3872), QMPS (SEQ ID NO: 3873), QGSL (SEQ ID NO: 3874), QVPA (SEQ ID NO: 3875), HYQQ (SEQ ID NO: 3876), QVPS (SEQ ID NO: 3877), RGEQ (SEQ ID NO: 3878), PGQQ (SEQ ID NO: 3879), LEQQ (SEQ ID NO: 3880), QNQS (SEQ ID NO: 3881), QKVI (SEQ ID NO: 3882), QNND (SEQ ID NO: 3883), QSVH (SEQ ID NO: 3884), QPLG (SEQ ID NO: 3885), HNQE (SEQ ID NO: 3886), QIQQ (SEQ ID NO: 3887), QVRN (SEQ ID NO: 3888), PSNQ (SEQ ID NO: 3889), QVGH (SEQ ID NO: 3890), QRDI (SEQ ID NO: 3891), QMPN (SEQ ID NO: 3892), RGLQ (SEQ ID NO: 3893), PSLQ (SEQ ID NO: 3894), QRDQ (SEQ ID NO: 3895), QAKG (SEQ ID NO: 3896), QSAH (SEQ ID NO: 3897), QSTM (SEQ ID NO: 3898), QREM (SEQ ID NO: 3899), QYRA (SEQ ID NO: 3900), QRQQ (SEQ ID NO: 3901), QWQQ (SEQ ID NO: 3902), QRMN (SEQ ID NO: 3903), GDSQ (SEQ ID NO: 3904), QKIS (SEQ ID NO: 3905), PSMQ (SEQ ID NO: 3906), SPRQ (SEQ ID NO: 3907), MEQQ (SEQ ID NO: 3908), QYQN (SEQ ID NO: 3909), QIRQ (SEQ ID NO: 3910), QSVQ (SEQ ID NO: 3911), RSQQ (SEQ ID NO: 3912), QNKL (SEQ ID NO: 3913), QIQH (SEQ ID NO: 3914), PRQQ (SEQ ID NO: 3915), HTQQ (SEQ ID NO: 3916), QRQH (SEQ ID NO: 3917), RNQE (SEQ ID NO: 3918), QSKQ (SEQ ID NO: 3919), QNQP (SEQ ID NO: 3920), QSPQ (SEQ ID NO: 3921), QTRQ (SEQ ID NO: 3922), QNLH (SEQ ID NO: 3923), QNQE (SEQ ID NO: 3924), LNQP (SEQ ID NO: 3925), QNQD (SEQ ID NO: 3926), QNLL (SEQ ID NO: 3927), QLVI (SEQ ID NO: 3928), RTQE (SEQ ID NO: 3929), QTHQ (SEQ ID NO: 3930), QDQH (SEQ ID NO: 3931), QSQH (SEQ ID NO: 3932), VRQQ (SEQ ID NO: 3933), AWQQ (SEQ ID NO: 3934), QSVP (SEQ ID NO: 3935), QNIQ (SEQ ID NO: 3936), LDQQ (SEQ ID NO: 3937), PDQQ (SEQ ID NO: 3938), ESQQ (SEQ ID NO: 3939), QRQL (SEQ ID NO: 3940), QIIV (SEQ ID NO: 3941), QKQS (SEQ ID NO: 3942), QSHQ (SEQ ID NO: 3943), QFVV (SEQ ID NO: 3944), QSQP (SEQ ID NO: 3945), QNEQ (SEQ ID NO: 3946), INQQ (SEQ ID NO: 3947), RNRQ (SEQ ID NO: 3948), RDQK (SEQ ID NO: 3949), QWKR (SEQ ID NO: 3950), ENRQ (SEQ ID NO: 3951), QTQP (SEQ ID NO: 3952), QKQL (SEQ ID NO: 3953), RNQL (SEQ ID NO: 3954), ISIQ (SEQ ID NO: 3955), QTVC (SEQ ID NO: 3956), QQIM (SEQ ID NO: 3957), LNHQ (SEQ ID NO: 3958), QNQA (SEQ ID NO: 3959), QMIH (SEQ ID NO: 3960), RNHQ (SEQ ID NO: 3961), or QKMN (SEQ ID NO: 3962), or any dipeptide or tripeptide thereof. In some embodiments, [N1]-[N2]-[N3]-[N4] is or comprises: the amino acid sequence of any of SEQ ID NOs: 1800-2241; an amino acid sequence comprising any portion of any of the aforesaid amino acid sequences (e.g., any 2, 3, 4, 5, 6, 7, 8, 9, 10, 11, or 12 amino acids, e.g., consecutive amino acids) thereof; an amino acid sequence comprising one, two, or three but no more than four modifications, e.g., substitutions (e.g., conservative substitutions), insertions, or deletions, relative to any of the aforesaid amino acid sequences; or an amino acid sequence comprising one, two, or three but no more than four different amino acids, relative to any one of the aforesaid amino acid sequences. In some embodiments, [N1]-[N2]-[N3]-[N4] is or comprises GSGSPHSKAQNQQ (SEQ ID NO: 1801). In some embodiments, [N1]-[N2]-[N3]-[N4] is or comprises GHDSPHKSGQNQQ (SEQ ID NO: 1800).

In some embodiments, the AAV capsid variant comprising an amino acid sequence having the formula [N1]-[N2]-[N3], further comprises [N0], wherein [N0] comprises X_{A} XB and XC. In some embodiments, X_{A} of [N0] is: T, S, Y, M, A, C, I, R, L, D, F, V, Q, N, H, E, or G. In some embodiments, XB of [N0] is: I, M, P, E, N, D, S, A, T, G, Q, F, V, L, C, H, R, W, or L. In some embodiments, X_{C} of [N0] is: N, M, E, G, Y, W, T, I, Q, F, V, A, L, I, P, K, R, H, S, D, or S. In some embodiments, [N0] comprises TIN, SMN, TIM, YLS, GLS, MPE, MEG, MEY, AEW, CEW, ANN, IPE, ADM, IEY, ADY, IET, MEW, CEY, RIN, MEI, LEY, ADW, IEI, DIM, FEQ, MEF, CDQ, LPE, IEN, MES, AEI, VEY, IIN, TSN, IEV, MEM, AEV, MDA, VEW, AEQ, LEW, MEL, MET, MEA, IES, MEV, CEI, ATN, MDG, QEV, ADQ, NMN, IEM, ISN, TGN, QQQ, HDW, IEG, TII, TFP, TEK, EIN, TVN, TFN, SIN, TER, TSY, ELH, AlN, SVN, TDN, TFH, TVH, TEN, TSS, TID, TCN, NIN, TEH, AEM, AIK, TDK, TFK, SDQ, TEI, NTN, TET, SIK, TEL, TEA, TAN, TIY, TFS, TES, TTN, TED, TNN, EVH, TIS, TVR, TDR, TIK, NHI, TIP, ESD, TDL, TVP, TVI, AEH, NCL, TVK, NAD, TIT, NCV, TIR, NAL, VIN, TIQ, TEF, TRE, QGE, SEK, NVN, GGE, EFV, SDK, TEQ, EVQ, TEY, NCW, TDV, SDI, NSI, NSL, EVV, TEP, SEL, TWQ, TEV, AVN, GVL, TLN, TEG, TRD, NAI, AEN, AET, ETA, NNL, or any dipeptide thereof. In some embodiments, [N0]-[N1]-[N2]-[N3]-[N4] is or comprises the amino acid sequence of any one of SEQ ID NOs: 2242-2886; an amino acid sequence comprising any portion of any of the aforesaid amino acid sequences (e.g., any 2, 3, 4, 5, 6, 7, 8, 9, 10, 11, 12, 13, 14, or 15 amino acids, e.g., consecutive amino acids) thereof; an amino acid sequence comprising one, two, or three but no more than four modifications, e.g., substitutions (e.g., conservative substitutions), insertions, or deletions, relative to any of the aforesaid amino acid sequences; or an amino acid sequence comprising one, two, or three but no more than four different amino acids, relative to any one of the aforesaid amino acid sequences. In some embodiments, [N0]-[N1]-[N2]-[N3]-[N4] is or comprises TINGSGSPHSKAQNQQ (SEQ ID NO: 2242). In some embodiments, [N0]-[N1]-[N2]-[N3]-[N4] is or comprises TINGHDSPHKSGQNQQ (SEQ ID NO: 2243).

In some embodiments, [N1]-[N2]-[N3] is present in loop IV of the AAV capsid variant. In some embodiments [N0] and [N4] are present in loop IV of the AAV capsid variant. In some embodiments, [N0]-[N1]-[N2]-[N3]-[N4] is present in loop IV of the AAV capsid variant.

In some embodiments, [N0] is present immediately subsequent to position 449, relative to a reference sequence numbered according to the amino acid sequence of SEQ ID NO: 138. In some embodiments, wherein [N0] is present immediately subsequent to position 449, relative to a reference sequence numbered according to the amino acid sequence of SEQ ID NO: 981 or 982. In some embodiments, [N0] replaces positions 450, 451, and 452 (e.g., amino acids T450, 1451, and N452), relative to a reference sequence numbered according to SEQ ID NO: 138, 981, or 982, wherein [N0] is present immediately subsequent to position 449 and wherein [N0] replaces positions 450-452 (e.g., T450, 1451, and N452), relative to a reference sequence numbered according to SEQ ID NO: 138, 981, or 982. In some embodiments, [N1] is present immediately subsequent to position 452, relative to a reference sequence numbered according to the amino acid sequence of SEQ ID NO: 138, 981 or 982. In some embodiments, wherein [N1] replaces positions 453-455 (e.g., G453, S454, and G455), relative to a reference sequence numbered according to SEQ ID NO: 138, 981, or 982. In some embodiments, [N1] is present immediately subsequent to position 452 and wherein [N1] replaces positions 453-455 (e.g., G453, S454, and G455), relative to a reference sequence numbered according to SEQ ID NO: 138, 981, or 982. In some embodiments, [N2] is present immediately subsequent to position 455, relative to a reference sequence numbered according to the amino acid sequence of SEQ ID NO: 138, 981, or 982. In some embodiments, [N2]-[N3] is present immediately subsequent to position 455, relative to a reference sequence numbered according to the amino acid sequence of SEQ ID NO: 138, 981, or 982. In some embodiments [N1]-[N2]-[N3] is present immediately subsequent to position 452, numbered relative to SEQ ID NO: 138, 981, or 982. In some embodiments, [N1]-[N2]-[N3] replaces positions 453-455 (e.g., G453, S454, and G455), relative to a reference sequence numbered according to SEQ ID NO: 138, 981, or 982. In some embodiments, [N1] is present immediately subsequent to position 452 and wherein [N1]-[N2]-[N3] replaces positions 453-455 (e.g., G453, S454, and G455), relative to a reference sequence numbered according to SEQ ID NO: 138, 981, or 982. In some embodiments, [N4] is present immediately subsequent to position 455, relative to a reference sequence numbered according to the amino acid sequence of SEQ ID NO: 138. In some embodiments, [N4] replaces positions 456-459 (e.g., Q456, N457, Q458, and Q459), relative to a reference sequence numbered according to the amino acid sequence of SEQ ID NO: 138. In some embodiments, [N4] is present immediately subsequent to position 455, and [N4] replaces positions 456-459 (e.g., Q456, N457, Q458, and Q459), relative to a reference sequence numbered according to the amino acid sequence of SEQ ID NO: 138. In some embodiments, [N2]-[N3]-[N4] replaces positions 456-459 (e.g., Q456, N457, Q458, and Q459), relative to a reference sequence numbered according to the amino acid sequence of SEQ ID NO: 138. In some embodiments, [N2]-[N3]-[N4] is present immediately subsequent to position 455, and wherein [N2]-[N3]-[N4] replaces positions 456-459 (e.g., Q456, N457, Q458, and Q459), relative to a reference sequence numbered according to the amino acid sequence of SEQ ID NO: 138. In some embodiments, [N1]-[N2]-[N3]-[N4] replaces positions 453-459 (e.g., G453, S454, G455, Q456, N457, Q458, and Q459), relative to a reference sequence numbered according to the amino acid sequence of SEQ ID NO: 138. In some embodiments, [N1]-[N2]-[N3]-[N4] is present immediately subsequent to position 452, and wherein [N1]-[N2]-[N3]-[N4] replaces positions 453-459 (e.g., G453, S454, G455, Q456, N457, Q458, and Q459), relative to a reference sequence numbered according to the amino acid sequence of SEQ ID NO: 138. In some embodiments, [N0]-[N1]-[N2]-[N3]-[N4] replaces positions 450-459 (e.g., T450, I451, N452, G453, S454, G455, Q456, N457, Q458, and Q459), relative to a reference sequence numbered according to the amino acid sequence of SEQ ID NO: 138. In some embodiments, [N0]-[N1]-[N2]-[N3]-[N4] is present immediately subsequent to position 449, and wherein [N0]-[N1]-[N2]-[N3]-[N4] replaces positions 450-459 (e.g., T450, 1451, N452, G453, S454, G455, Q456, N457, Q458, and Q459), relative to a reference sequence numbered according to the amino acid sequence of SEQ ID NO: 138.

In some embodiments, [N3] is present immediately subsequent to [N2].

In some embodiments, the AAV capsid variant comprises from N-terminus to C-terminus, [N2]-[N3]. In some embodiments, the AAV capsid variant comprises from N-terminus to C-terminus, [N1]-[N2]-[N3]. In some embodiments, the AAV capsid variant comprises from N-terminus to C-terminus, [N1]-[N2]-[N3]-[N4]. In some embodiments, the AAV capsid variant comprises from N-terminus to C-terminus, [N0]-[N1]-[N2]-[N3]. In some embodiments, the AAV capsid variant comprises from N-terminus to C-terminus, [N0]-[N1]-[N2]-[N3]-[N4].

In some embodiments, an AAV capsid variant described herein comprises an amino acid sequence comprising at least 3, 4, 5, 6, 7, 8, 9, 10, 11, 12, 13, 16, or 17 consecutive amino acids from any one of the sequences provided in Tables 1A, 2A, 2B, 21-23, 25, 26, or 30. In some embodiments, the AAV capsid variant comprises an amino acid sequence comprising at least 3, 4, or 5 consecutive amino acids from any one of SEQ ID NOs: 945-980 or 985-986. In some embodiments, the AAV capsid variant comprises an amino acid sequence comprising at least 3, 4, 5, 6, 7, 8, 9, 10, 11, 12, or 13 consecutive amino acids from any one of SEQ ID NOs: 200, 201, 941, 943, 204, 208, 404, or 903-909. In some embodiments, the amino acid sequence is present in loop IV. In some embodiments, the amino acid sequence is present immediately subsequent to position 448, 452, 453, 455, relative to a reference sequence numbered according to the amino acid sequence of SEQ ID NO: 138, 981, or 982. In some embodiments, the amino acid sequence is present immediately subsequent to position 455, numbered according to SEQ ID NO: 982. In some embodiments, the amino acid sequence is present immediately subsequent to position 455, numbered according to SEQ ID NO: 138. In some embodiments, the amino acid sequence is present immediately subsequent to position 453, numbered according to SEQ ID NO: 981. In some embodiments, the amino acid sequence is present immediately subsequent to position 453, numbered according to SEQ ID NO: 138. In some embodiments, the amino acid sequence replaces 1, 2, 3, 4, 5, 6, 7, 8, 9, 10, 11, or all of positions 499 (e.g., K499), 450 (e.g., T450), 451 (e.g., 1451), 452 (e.g., N452), 453 (e.g., G453), 454 (e.g., S454), 455 (e.g., G455), 456 (e.g., Q456), 457 (e.g., N457), 458 (e.g., Q458), 459 (e.g., Q459), and 460 (e.g., T460), numbered according to SEQ ID NO: 138. In some embodiments, the AAV capsid variant comprises one or more amino acid substitutions at positions 499 (e.g., K499), 450 (e.g., T450), 451 (e.g., 1451), 452 (e.g., N452), 453 (e.g., G453), 454 (e.g., S454), 455 (e.g., G455), 456 (e.g., Q456), 457 (e.g., N457), 458 (e.g., Q458), 459 (e.g., Q459), and 460 (e.g., T460), numbered according to SEQ ID NO: 138.

In some embodiments, the at least 3 consecutive amino acids comprise SPH. In some embodiments, the at least 4 consecutive amino acids comprise SPHS (SEQ ID NO: 1371). In some embodiments, the at least 5 consecutive amino acids comprise SPHSK (SEQ ID NO: 1372). In some embodiments, the at least 6 consecutive amino acids comprise SPHSKA (SEQ ID NO: 941).

In some embodiments, the amino acid sequence of SPHSKA (SEQ ID NO: 941) is present at amino acids 456-461, numbered according to SEQ ID NO: 981. In some embodiments, the amino acid sequence of SPHSKA (SEQ ID NO: 941) is present at amino acids 456-461, numbered according to SEQ ID NO: 69. In some embodiments, the amino acid sequence of SPHSKA (SEQ ID NO: 941) is present at amino acids 456-461, numbered according to SEQ ID NO: 36.

In some embodiments, the at least 3 consecutive amino acids comprise HDS. In some embodiments, the at least 4 consecutive amino acids comprise HDSP (SEQ ID NO: 1373). In some embodiments, the at least 5 consecutive amino acids comprise HDSPH (SEQ ID NO: 1374). In some embodiments, the at least 6 consecutive amino acids comprise HDSPHK (SEQ ID NO: 2).

In some embodiments, the amino acid sequence of HDSPHK (SEQ ID NO: 2) is present at amino acids 454-459, numbered according to SEQ ID NO: 982.

In some embodiments, an AAV capsid variant described herein comprises an amino acid sequence comprising at least one, two, or three but no more than four modifications, e.g., substitutions (e.g., conservative substitutions), insertions, or deletions, relative to the amino acid sequence of any one of the sequences provided in Tables 1A, 2A, 2B, 21-23, 25, 26, or 30. In some embodiments, the AAV capsid variant comprises an amino acid sequence comprising at least one, two, or three but no more than four different amino acids, relative to the amino acid sequence of any one of the sequences provided in Tables 1A, 2A, 2B, 21-23, 25, 26, or 30. In some embodiments, the AAV capsid variant comprises an amino acid sequence comprising at least one, two, or three but no more than four modifications, e.g., substitutions (e.g., conservative substitutions), insertions, or deletions, relative to the amino acid sequence of any one of SEQ ID NOs: 945-980 or 985-986. In some embodiments, the AAV capsid variant comprises an amino acid sequence comprising at least one, two, or three but no more than four different amino acids, relative to the amino acid sequence of any one of SEQ ID NOS: 945-980 or 985-986. In some embodiments, the AAV capsid variant comprises an amino acid sequence comprising at least one, two, or three but no more than four modifications, e.g., substitutions (e.g., conservative substitutions), insertions, or deletions, relative to the amino acid sequence of any one of SEQ ID NOs: 2, 200, 201, 941, 943, 204, 208, 404, or 903-909. In some embodiments, the AAV capsid variant comprises an amino acid sequence comprising at least one, two, or three but no more than four different amino acids, from the amino acid sequence of any one of SEQ ID NOs: 2, 200, 201, 941, 943, 204, 208, 404, or 903-909. In some embodiments, the amino acid sequence is present in loop IV. In some embodiments, the amino acid sequence is present immediately subsequent to position 448, 452, 453, 455, relative to a reference sequence numbered according to the amino acid sequence of SEQ ID NO: 138, 981, or 982. In some embodiments, the amino acid sequence is present immediately subsequent to position 455, numbered according to SEQ ID NO: 982. In some embodiments, the amino acid sequence is present immediately subsequent to position 455, numbered according to SEQ ID NO: 138. In some embodiments, the amino acid sequence is present immediately subsequent to position 453, numbered according to SEQ ID NO: 981. In some embodiments, the amino acid sequence is present immediately subsequent to position 453, numbered according to SEQ ID NO: 138. In some embodiments, the amino acid sequence replaces 1, 2, 3, 4, 5, 6, 7, 8, 9, 10, 11, or all of positions 499 (e.g., K499), 450 (e.g., T450), 451 (e.g., 1451), 452 (e.g., N452), 453 (e.g., G453), 454 (e.g., S454), 455 (e.g., G455), 456 (e.g., Q456), 457 (e.g., N457), 458 (e.g., Q458), 459 (e.g., Q459), and 460 (e.g., T460), numbered according to SEQ ID NO: 138.

In some embodiments, the AAV capsid variant comprises an amino acid sequence comprising at least one, two, or three but no more than four modifications, e.g., substitutions (e.g., conservative substitutions), insertions, or deletions, relative to the amino acid sequence of SPHSKA (SEQ ID NO: 941). In some embodiments, the AAV capsid variant comprises an amino acid sequence comprising at least one, two, or three, but no more than four different amino acids from the amino acid sequence of SPHSKA (SEQ ID NO: 941).

In some embodiments, the AAV capsid variant comprises an amino acid sequence comprising at least one, two, or three but no more than four modifications, e.g., substitutions (e.g., conservative substitutions), insertions, or deletions, relative to the amino acid sequence of HDSPHK (SEQ ID NO: 2). In some embodiments, the AAV capsid variant comprises an amino acid sequence comprising at least one, two, or three, but no more than four different amino acids that relative to the amino acid sequence of HDSPHK (SEQ ID NO: 2).

In some embodiments, the AAV capsid variant, comprises the amino acid sequence of any of the sequences provided in Tables 1A, 2A, 2B, 21-23, 25, 26, or 30. In some embodiments, the peptide comprises the amino acid sequence of any of SEQ ID NOs: 945-980 or 985-986. In some embodiments, the AAV capsid variant comprises the amino acid sequence of any of SEQ ID NOs: 2, 200, 201, 941, 943, 204, 208, 404, or 903-909. In some embodiments, the AAV capsid variant comprises the amino acid sequence of SEQ ID NO: 2. In some embodiments, the AAV capsid variant comprises the amino acid sequence of SEQ ID NO: 941. In some embodiments, the AAV capsid variant comprises the amino acid sequence of SEQ ID NO: 943. In some embodiments, the AAV capsid variant comprises the amino acid sequence of SEQ ID NO: 3589. In some embodiments, the AAV capsid variant comprises the amino acid sequence of SEQ ID NO: 1754. In some embodiments, the AAV capsid variant comprises the amino acid sequence of SEQ ID NO: 3241. In some embodiments, the AAV capsid variant comprises the amino acid sequence of SEQ ID NO: 4100. In some embodiments, the AAV capsid variant comprises the amino acid sequence of SEQ ID NO: 4062. In some embodiments, the AAV capsid variant comprises the amino acid sequence of SEQ ID NO: 4486. In some embodiments, the amino acid sequence is present in loop IV. In some embodiments, the amino acid sequence is present immediately subsequent to position 448, relative to a reference sequence numbered according to the amino acid sequence of SEQ ID NO: 138. In some embodiments, the amino acid sequence replaces positions 449-460 (e.g., K449, T450, 1451, N452, G453, S454, G455, Q456, N457, Q458, Q459, and T460), numbered relative to SEQ ID NO: 138. In some embodiments, the amino acid sequence is present immediately subsequent to position 448 and replaces positions 449-460 (e.g., K449, T450, 1451, N452, G453, S454, G455, Q456, N457, Q458, Q459, and T460), numbered relative to SEQ ID NO: 138. In some embodiments, the amino acid sequence is present immediately subsequent to position 449, relative to a reference sequence numbered according to the amino acid sequence of SEQ ID NO: 138. In some embodiments, the amino acid sequence replaces positions 450-460 (e.g., T450, 1451, N452, G453, S454, G455, Q456, N457, Q458, Q459, and T460), numbered relative to SEQ ID NO: 138. In some embodiments, the amino acid sequence is present immediately subsequent to position 449, and replaces positions 450-460 (e.g., T450, 1451, N452, G453, S454, G455, Q456, N457, Q458, Q459, and T460), numbered relative to SEQ ID NO: 138. In some embodiments, the amino acid sequence is present immediately subsequent to position 450, relative to a reference sequence numbered according to the amino acid sequence of SEQ ID NO: 138. In some embodiments, the amino acid sequence replaces positions 451-460 (e.g., 1451, N452, G453, S454, G455, Q456, N457, Q458, Q459, and T460), numbered relative to SEQ ID NO: 138. In some embodiments, the amino acid sequence is present immediately subsequent to position 450 and replaces positions 451-460 (e.g., 1451, N452, G453, S454, G455, Q456, N457, Q458, Q459, and T460), numbered relative to SEQ ID NO: 138. In some embodiments, the amino acid sequence is present immediately subsequent to position 451, relative to a reference sequence numbered according to the amino acid sequence of SEQ ID NO: 138. In some embodiments, the amino acid sequence replaces positions 452-460 (e.g., N452, G453, S454, G455, Q456, N457, Q458, Q459, and T460), numbered relative to SEQ ID NO: 138. In some embodiments, the amino acid sequence is present immediately subsequent to position 451 and replaces positions 452-460 (e.g., N452, G453, S454, G455, Q456, N457, Q458, Q459, and T460), numbered relative to SEQ ID NO: 138. In some embodiments, the amino acid sequence is present immediately subsequent to position 452, relative to a reference sequence numbered according to the amino acid sequence of SEQ ID NO: 138. In some embodiments, the amino acid sequence replaces positions 453-460 (e.g., G453, S454, G455, Q456, N457, Q458, Q459, and T460), numbered relative to SEQ ID NO: 138. In some embodiments, the amino acid sequence is present immediately subsequent to position 452, and replaces positions 453-460 (e.g., G453, S454, G455, Q456, N457, Q458, Q459, and T460), numbered relative to SEQ ID NO: 138. In some embodiments, the amino acid sequence is present immediately subsequent to position 453, relative to a reference sequence numbered according to the amino acid sequence of SEQ ID NO: 138. In some embodiments, the amino acid sequence replaces positions 454 and 455 (e.g., S454 and G455), numbered according to SEQ ID NO: 138. In some embodiments, the amino acid sequence is present immediately subsequent to position 453, and replaces positions 454 and 455 (e.g., S454 and G455), numbered according to SEQ ID NO: 138. In some embodiments, the amino acid sequence replaces positions 454-460 (e.g., S454, G455, Q456, N457, Q458, Q459, and T460), numbered relative to SEQ ID NO: 138. In some embodiments, the amino acid sequence is present immediately subsequent to position 453, and replaces positions 454-460 (e.g., S454, G455, Q456, N457, Q458, Q459, and T460), numbered relative to SEQ ID NO: 138. In some embodiments, the amino acid sequence is present immediately subsequent to position 454, relative to a reference sequence numbered according to the amino acid sequence of SEQ ID NO: 138. In some embodiments, the amino acid sequence is present immediately subsequent to position 454, relative to a reference sequence numbered according to the amino acid sequence of SEQ ID NO: 981. In some embodiments, the amino acid sequence replaces positions 455-460 (e.g., positions G455, Q456, N457, Q458, Q459, and T460), numbered relative to SEQ ID NO: 138. In some embodiments, the amino acid sequence is present immediately subsequent to positions 454, and replaces positions 455-460 (e.g., positions G455, Q456, N457, Q458, Q459, and T460), numbered relative to SEQ ID NO: 138. In some embodiments, the amino acid sequence is present immediately subsequent to position 455, relative to a reference sequence numbered according to the amino acid sequence of SEQ ID NO: 138. In some embodiments, the amino acid sequence is present immediately subsequent to position 455, relative to a reference sequence numbered according to the amino acid sequence of SEQ ID NO: 982. In some embodiments, the amino acid sequence replaces positions 456-460 (e.g., Q456, N457, Q458, Q459, and T460), numbered relative to SEQ ID NO: 138. In some embodiments, the amino acid sequence is present immediately subsequent to position 455, and replaces positions 456-460 (e.g., Q456, N457, Q458, Q459, and T460), numbered relative to SEQ ID NO: 138.

In some embodiments, the AAV capsid variant (e.g., an AAV capsid variant described herein), comprises an amino acid sequence encoded by the nucleotide sequence of SEQ ID NO: 3 or 942, or a nucleotide sequence substantially identical (e.g., having at least 70%, 75%, 80%, 85%, 90%, 92%, 95%, 97%, 98%, or 99% sequence identity) thereto. In some embodiments, the AAV capsid variant described herein, comprises an amino acid sequence encoded by the nucleotide sequence of SEQ ID NO: 3 or 942, or a nucleotide sequence comprising at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, relative to the nucleotide sequence of SEQ ID NO: 3 or 942. In some embodiments, the AAV capsid variant comprises an amino acid sequence encoded by a nucleotide sequence comprising at least one, two, three, four, five, six, or seven, but no more than ten different nucleotides relative to the nucleotide sequence of SEQ ID NO: 3 or 942.

In some embodiments, the nucleotide sequence encoding the AAV capsid variant (e.g., an AAV capsid variant described herein), comprises the nucleotide sequence of SEQ ID NO: 942, or a nucleotide sequence substantially identical (e.g., having at least 70%, 75%, 80%, 85%, 90%, 92%, 95%, 97%, 98%, or 99% sequence identity) thereto. In some embodiments, the nucleic acid sequence encoding the AAV capsid variant comprises a nucleotide sequence comprising at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, relative to the nucleotide sequences of SEQ ID NO: 942. In some embodiments, the nucleotide sequence encoding an AAV capsid variant described herein comprises a nucleotide sequence comprising at least one, two, three, four, five, six, or seven, but no more than ten different nucleotides, relative to the nucleotide sequence of SEQ ID NO: 942.

In some embodiments, the nucleotide sequence encoding the AAV capsid variant (e.g., an AAV capsid variant described herein), comprises the nucleotide sequence of SEQ ID NO: 3, or a nucleotide sequence substantially identical (e.g., having at least 70%, 75%, 80%, 85%, 90%, 92%, 95%, 97%, 98%, or 99% sequence identity) thereto. In some embodiments, the nucleic acid sequence encoding the AAV capsid variant comprises a nucleotide sequence comprising at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, relative to the nucleotide sequences of SEQ ID NO: 3. In some embodiments, the nucleotide sequence encoding an AAV capsid variant described herein comprises a nucleotide sequence comprising at least one, two, three, four, five, six, or seven, but no more than ten different nucleotides relative to the nucleotide sequence of SEQ ID NO: 3.

In some embodiments, an AAV capsid variant described herein comprises the amino acid sequence of SPHSKA (SEQ ID NO: 941), wherein the amino acid sequence is present immediately subsequent to position 455, relative to a reference sequence numbered according to the amino acid sequence of SEQ ID NO: 138. In some embodiments, an AAV capsid variant described herein comprises the amino acid sequence of SPHSKA (SEQ ID NO: 941), wherein the amino acid sequence is present immediately subsequent to position 455, relative to a reference sequence numbered according to the amino acid sequence of SEQ ID NO: 981.

In some embodiments, an AAV capsid variant described herein comprises the amino acid sequence of HDSPHK (SEQ ID NO: 2), wherein the amino acid sequence is present immediately subsequent to position 453, relative to a reference sequence numbered according to the amino acid sequence of SEQ ID NO: 138. In some embodiments, an AAV capsid variant described herein comprises the amino acid sequence of HDSPHK (SEQ ID NO: 2), wherein the amino acid sequence is present immediately subsequent to position 453, relative to a reference sequence numbered according to the amino acid sequence of SEQ ID NO: 982.

In some embodiments, an AAV capsid variant described herein comprises (i) the amino acid sequence of HDSPHK (SEQ ID NO: 2), which is present immediately subsequent to position 453; and (ii) a deletion of amino acids SG at position 454 and 455; wherein (i) and (ii) are numbered according to SEQ ID NO: 138.

In some embodiments, an AAV capsid variant described herein comprises an amino acid other than S at position 454 and/or an amino acid other than G at position 455, numbered according to SEQ ID NO: 138. In some embodiments, the AAV capsid variant comprises the amino acid H at position 454 and the amino acid D at position 455, numbered according to SEQ ID NO: 138. In some embodiments, the AAV capsid variant further comprises the amino acid sequence of SPHSKA (SEQ ID NO: 941). In some embodiments, the AAV capsid variant comprises: (i) the amino acid H at position 454 and the amino acid D at position 455, and (ii) the amino acid sequence SPHSKA (SEQ ID NO: 941), wherein the amino acid sequence of SPHSKA (SEQ ID NO: 941) is present immediately subsequent to position 455, wherein (i) and (ii) are numbered according to SEQ ID NO: 138.

In some embodiments, an AAV capsid variant described herein comprises a modification, e.g., substitution, relative to SEQ ID NO: 138. In some embodiments, the AAV capsid variant comprises a modification, e.g., substitution, at position S454 and/or G455, numbered relative to SEQ ID NO: 138. In some embodiments, the AAV capsid variant comprises a S454H substitution and/or G455D substitution, numbered relative to SEQ ID NO: 138. In some embodiments, the AAV capsid variant comprises a S454H substitution and a G455D substitution, numbered relative to SEQ ID NO: 138. In some embodiments, the AAV capsid variant further comprises the amino acid sequence of SPHSKA (SEQ ID NO: 941). In some embodiments, the AAV capsid variant comprises: (i) a S454H substitution and a G455D substitution, and (ii) the amino acid sequence SPHSKA (SEQ ID NO: 941), wherein the amino acid sequence of SPHSKA (SEQ ID NO: 941) is present immediately subsequent to position 455, wherein (i) and (ii) are numbered according to SEQ ID NO: 138.

In some embodiments, the AAV capsid variant further comprises one, two, or all of an amino acid other than T at position 450 (e.g., S, Y, or G), an amino acid other than I at position 451 (e.g., M or L), and/or an amino acid other than N at position 452 (e.g., S), relative to a reference sequence numbered according to the amino acid sequence of SEQ ID NO: 138. In some embodiments, the AAV capsid variant further comprises an S at position 450 and an M at position 451, relative to a reference sequence numbered according to the amino acid sequence of SEQ ID NO: 138. In some embodiments, the AAV capsid variant further comprises a Y at position 450, an L at position 451, and an S at position 452, relative to a reference sequence numbered according to the amino acid sequence of SEQ ID NO: 138. In some embodiments, the AAV capsid variant further comprises a G at position 450, an L at position 451, and an S at position 452, relative to a reference sequence numbered according to the amino acid sequence of SEQ ID NO: 138.

In some embodiments, the AAV capsid variant further comprises one, two, three, four, or all of an amino acid other than Q at position 456 (e.g., R or L), N at position 457 (e.g., H, K, or R), Q at position 458 (e.g., R or T), Q at position 459 (H), and/or T at position 460 (N or S), relative to a reference sequence numbered according to the amino acid sequence of SEQ ID NO: 138. In some embodiments, the AAV capsid variant further comprises an R at position 456, relative to a reference sequence numbered according to the amino acid sequence of SEQ ID NO: 138. In some embodiments, the AAV capsid variant further comprises an L at position 456, relative to a reference sequence numbered according to the amino acid sequence of SEQ ID NO: 138. In some embodiments, the AAV capsid variant further comprises an H at position 457 and an R at position 458, relative to a reference sequence numbered according to the amino acid sequence of SEQ ID NO: 138. In some embodiments, the AAV capsid variant further comprises a K at position 457 and an N at position 460, relative to a reference sequence numbered according to the amino acid sequence of SEQ ID NO: 138. In some embodiments, the AAV capsid variant further comprises a T at position 458, an H at position 459, and an S at position 460, relative to a reference sequence numbered according to the amino acid sequence of SEQ ID NO: 138. In some embodiments, the AAV capsid variant further comprises an R at position 456, an R at position 457, and an R at position 458, relative to a reference sequence numbered according to the amino acid sequence of SEQ ID NO: 138.

In some embodiments, an AAV capsid variant described herein comprises an amino acid other than I at position 451, an amino acid other than N at position 452, and an amino acid other than G at position 453, numbered according to SEQ ID NO: 138 or 981. In some embodiments, the AAV capsid variant comprises E at position 451, R at position 452, and V at position 453, numbered according to SEQ ID NO: 138 or 981. In some embodiments, the AAV capsid variant comprises the substitutions I451E, N452R, and G453V, numbered according to SEQ ID NO: 138 or 981.

In some embodiments, the AAV capsid variant comprises the amino acid sequence of SPHSKA (SEQ ID NO: 941), wherein the amino acid sequence is present immediately subsequent to position 455 and wherein the AAV capsid variant comprises the E at position 451, R at position 452, and V at position 453, numbered according to the amino acid sequence of SEQ ID NO: 138 or 981. In some embodiments, the AAV capsid variant comprises the substitutions I451E, N452R, and G453V, and further comprises the amino acid sequence of SPHSKA (SEQ ID NO: 941), wherein the amino acid sequence is present immediately subsequent to position 455, all numbered according to SEQ ID NO: 138 or 981. In some embodiments, the AAV capsid variant comprises the amino acid sequence of ERVSGSPHSKA (SEQ ID NO: 4817), and wherein the amino acid sequence is present immediately subsequent to position 450 and replaces positions 451-455, numbered according to SEQ ID NO: 138. In some embodiments, the AAV capsid variant comprises the amino acid sequence of KTERVSGSPHSKAQNQQT (SEQ ID NO: 3589), wherein the amino acid sequence is present immediately subsequent to position 448 and replaces positions 449-460, numbered according to SEQ ID NO: 138.

In some embodiments, the AAV capsid variant comprises: (i) the amino acid sequence of SPHSKA (SEQ ID NO: 941), wherein the amino acid sequence is present immediately subsequent to position 455, numbered according to SEQ ID NO: 138, 981, or 69; and (ii) one or both of E at position 451 and/or V at position 453, numbered according to the amino acid sequence of SEQ ID NO: 69, 138, or 981.

In some embodiments, the AAV capsid variant comprises: (i) the amino acid sequence of SPHSKA (SEQ ID NO: 941), wherein the amino acid sequence is present immediately subsequent to position 455, numbered according to SEQ ID NO: 138, 981, or 69; and (ii) E at position 451 and V at position 453, numbered according to the amino acid sequence of SEQ ID NO: 69, 138, or 981.

In some embodiments, the AAV capsid variant comprises: (i) the amino acid sequence of SPHSKA (SEQ ID NO: 941), wherein the amino acid sequence is present at amino acids 456-461, numbered according to SEQ ID NO: 981 or 69; and (ii) one or both of E at position 451 and/or V at position 453, numbered according to the amino acid sequence of SEQ ID NO: 69, 138, or 981.

In some embodiments, the AAV capsid variant comprises: (i) the amino acid sequence of SPHSKA (SEQ ID NO: 941), wherein the amino acid sequence is present at amino acids 456-461, numbered according to SEQ ID NO: 981 or 69; and (ii) E at position 451 and V at position 453, numbered according to the amino acid sequence of SEQ ID NO: 69, 138, or 981.

In some embodiments, the AAV capsid variant comprises: (i) the amino acid sequence of SPHSKA (SEQ ID NO: 941), wherein the amino acid sequence is present at amino acids 456-461, numbered according to SEQ ID NO: 981 or 36; and (ii) one, two, or all of E at position 451, R at position 452, and/or V at position 453, numbered according to the amino acid sequence of SEQ ID NO: 36, 138, or 981.

In some embodiments, the AAV capsid variant comprises: (i) the amino acid sequence of SPHSKA (SEQ ID NO: 941), wherein the amino acid sequence is present at amino acids 456-461, numbered according to SEQ ID NO: 981 or 36; and (ii) E at position 451, R at position 452, and V at position 453, numbered according to the amino acid sequence of SEQ ID NO: 36, 138, or 981.

In some embodiments, an AAV capsid variant described herein comprises an amino acid other than T at position 450, an amino acid other than I at position 451, and an amino acid other than N at position 452, numbered according to SEQ ID NO: 138 or 982. In some embodiments, the AAV capsid variant comprises A at position 450, E at position 451, and I at position 452, numbered according to SEQ ID NO: 138 or 982. In some embodiments, the AAV capsid variant comprises the substitutions T450A, I451E, and N452I, numbered according to SEQ ID NO: 138 or 982.

In some embodiments, the AAV capsid variant comprises the amino acid sequence of SPHKSG (SEQ ID NO: 946), which is present immediately subsequent to positions 455, and further comprises A at position 450, E at position 451, I at position 452, H at position 454, and D at position 455, all numbered according to SEQ ID NO: 138 or 982. In some embodiments, the AAV capsid variant comprises the substitutions T450A, I451E, or N452I, and further comprises the amino acid sequence HDSPHK (SEQ ID NO: 2) present immediately subsequent to position 453, all numbered according to SEQ ID NO: 138 or 982. In some embodiments, the AAV capsid variant comprises the amino acid sequence of AEIGHDSPHKSG (SEQ ID NO: 4818), wherein the amino acid sequence is present immediately subsequent to position 449 and replaces positions 450-455, numbered according to SEQ ID NO: 138. In some embodiments, the AAV capsid variant comprises the amino acid sequence of KAEIGHDSPHKSGQNQQT (SEQ ID NO: 1754), wherein the amino acid sequence is present immediately subsequent to position 448 and replaces positions 449-460, numbered according to SEQ ID NO: 138.

In some embodiments, the AAV capsid variant, further comprises a substitution at position K449, e.g., a K449R substitution, numbered according to SEQ ID NO: 138. In some embodiments, the AAV capsid variant, further comprises an amino acid other than K at position 449 (e.g., R), relative to a reference sequence numbered according to the amino acid sequence of SEQ ID NO: 138. In some embodiments, the AAV capsid variant comprises an R at position 449, relative to a reference sequence numbered according to the amino acid sequence of SEQ ID NO: 138. In some embodiments, the AAV capsid variant further comprises a modification, e.g., an insertion, substitution, and/or deletion in loop I, II, VI, and/or VIII.

In some embodiments, the AAV capsid variant, further comprises an amino acid sequence comprising at least one, two or three modifications, e.g., substitutions (e.g., conservative substitutions), insertions, or deletions, but not more than 30, 20 or 10 modifications, e.g., substitutions (e.g., conservative substitutions), insertions, or deletions, of the amino acid sequence of SEQ ID NO: 138. In some embodiments, the AAV capsid variant, further comprises an amino acid sequence comprising at least one, two or three, but not more than 30, 20 or 10 amino acids that differ from the amino acid sequence of SEQ ID NO: 138. In some embodiments, the AAV capsid variant further comprises the amino acid sequence of SEQ ID NO: 138, or an amino acid sequence with at least 70% (e.g., at least about 80, 85, 90, 95, 96, 97, 98, or 99%) sequence identity thereto.

In some embodiments, the AAV capsid variant further comprises (a) a VP1 protein comprising the amino acid sequence of SEQ ID NO: 138, 981, or 982; (b) a VP2 protein comprising the amino acid sequence of positions 138-736 of SEQ ID NO: 138 or positions 138-742 of SEQ ID NO: 981 or 982; (c) a VP3 protein comprising the amino acid sequence of positions 203-736 of SEQ ID NO: 138 or positions 203-742 of SEQ ID NO: 981 or 982; or (d) an amino acid sequence with at least 70% (e.g., at least about 80, 85, 90, 95, 96, 97, 98, or 99%) sequence identity to any of the amino acid sequences in (a)-(c), an amino acid sequence comprising at least one, two or three, but not more than 30, 20 or 10 different amino acids relative to any of the amino acid sequences in (a)-(c), or an amino acid sequence comprising at least one, two or three modifications, e.g., substitutions (e.g., conservative substitutions), insertions, or deletions, but not more than 30, 20 or 10 modifications, e.g., substitutions (e.g., conservative substitutions), insertions, or deletions, relative to any of the amino acid sequences in (a)-(c).

In some embodiments, the AAV capsid variant further comprises an amino acid sequence encoded by the nucleotide sequence of SEQ ID NO: 137, or a sequence with at least 70% (e.g., at least about 80, 85, 90, 95, 96, 97, 98, or 99%) sequence identity thereto. In some embodiments, the AAV capsid variant further comprises an amino acid sequence encoded by a nucleotide sequence comprising at least one, two or three modifications, e.g., substitutions (e.g., conservative substitutions), insertions, or deletions, but not more than 30, 20 or 10 modifications, e.g., substitutions (e.g., conservative substitutions), insertions, or deletions, relative to the nucleotide sequence of SEQ ID NO: 137. In some embodiments, the AAV capsid variant further comprises an amino acid sequence encoded by a nucleotide sequence comprising at least one, two or three, but not more than 30, 20 or 10 different nucleotides, relative to the amino acid sequence of SEQ ID NO: 137.

In some embodiments, the nucleotide sequence encoding the AAV capsid variant further comprises the nucleotide sequence of SEQ ID NO: 137, or a sequence with at least 70% (e.g., at least about 80, 85, 90, 95, 96, 97, 98, or 99%) sequence identity thereto. In some embodiments, the nucleotide sequence encoding the AAV capsid variant further comprises a nucleotide sequence comprising at least one, two or three modifications, e.g., substitutions (e.g., conservative substitutions), insertions, or deletions, but not more than 30, 20 or 10 modifications, e.g., substitutions (e.g., conservative substitutions), insertions, or deletions, relative to the nucleotide sequence of SEQ ID NO: 137. In some embodiments, the nucleotide sequence encoding the AAV capsid variant further comprises a nucleotide sequence comprising at least one, two or three, but not more than 30, 20 or 10 different nucleotides, relative to the amino acid sequence of SEQ ID NO: 137.

In some embodiments, an AAV capsid variant of the present disclosure comprises an amino acid sequence as described herein, e.g., an amino acid sequence of an AAV capsid variant of TTM-001 or TTM-002, e.g., as described in Tables 3 and 4. In some embodiments, an AAV capsid variant of the present disclosure comprises an amino acid sequence as described herein, e.g., an amino acid sequence of an AAV capsid variant of TTM-003, TTM-004, TTM-005, TTM-006, TTM-007, TTM-008, TTM-009, TTM-010, TTM-011, TTM-012, TTM-013, TTM-014, TTM-015, TTM-016, TTM-017, TTM-018, TTM-019, TTM-020, TTM-021, TTM-022, TTM-023, TTM-024, TTM-025, TTM-026, TTM-027, TTM-028, TTM-029, TTM-030, TTM-031, TTM-032, TTM-033, or TTM-034, e.g., as described in Table 4.

In some embodiments, an AAV capsid variant described herein comprises a VP1, VP2, and/or VP3 protein comprising an amino acid sequence described herein, e.g., an amino acid sequence of an AAV capsid variant of TTM-001 or TTM-002, e.g., as described in Tables 3 and 4. In some embodiments, an AAV capsid variant described herein comprises a VP1, VP2, and/or VP3 protein comprising an amino acid sequence described herein, e.g., an amino acid sequence of an AAV capsid variant of TTM-003, TTM-004, TTM-005, TTM-006, TTM-007, TTM-008, TTM-009, TTM-010, TTM-011, TTM-012, TTM-013, TTM-014, TTM-015, TTM-016, TTM-017, TTM-018, TTM-019, TTM-020, TTM-021, TTM-022, TTM-023, TTM-024, TTM-025, TTM-026, TTM-027, TTM-028, TTM-029, TTM-030, TTM-031, TTM-032, TTM-033, or TTM-034, e.g., as described in Table 4.

In some embodiments, an AAV capsid variant described herein comprises an amino acid sequence encoded by a nucleotide sequence as described herein, e.g., a nucleotide sequence of an AAV capsid variant of TTM-001 or TTM-002, e.g., as described in Tables 3 and 5. In some embodiments, an AAV capsid variant described herein comprises an amino acid sequence encoded by a nucleotide sequence as described herein, e.g., a nucleotide sequence of an AAV capsid variant of TTM-003, TTM-004, TTM-005, TTM-006, TTM-007, TTM-008, TTM-009, TTM-010, TTM-011, TTM-012, TTM-013, TTM-014, TTM-015, TTM-016, TTM-017, TTM-018, TTM-019, TTM-020, TTM-021, TTM-022, TTM-023, TTM-024, TTM-025, TTM-026, or TTM-027, e.g., as described in Table 5.

In some embodiments, a polynucleotide or nucleic acid encoding an AAV capsid variant, of the present disclosure comprises a nucleotide sequence described herein, e.g., a nucleotide sequence of an AAV capsid variant of TTM-001 or TTM-002, e.g., as described in Tables 3 and 5. In some embodiments, a polynucleotide or nucleic acid encoding an AAV capsid variant, of the present disclosure comprises a nucleotide sequence described herein, e.g., a nucleotide sequence of an AAV capsid variant of TTM-003, TTM-004, TTM-005, TTM-006, TTM-007, TTM-008, TTM-009, TTM-010, TTM-011, TTM-012, TTM-013, TTM-014, TTM-015, TTM-016, TTM-017, TTM-018, TTM-019, TTM-020, TTM-021, TTM-022, TTM-023, TTM-024, TTM-025, TTM-026, or TTM-027, e.g., as described in Table 5.

TABLE 3
Exemplary full length capsid sequences

| | VP1 DNA | VP1 | Peptide | Peptide DNA |
|---|---|---|---|---|
| | SEQ | (amino acid) | (amino acid) | SEQ ID |
| Name | ID NO: | SEQ ID NO: | SEQ ID NO: | NO: |
| TTM-001 | 983 | 981 | 941 | 942 |
| TTM-002 | 984 | 982 | 2 | 3 |

TABLE 4
Exemplary full length capsid amino acid sequences

| Name and | SEQ ID | |
|---|---|---|
| Annotation | NO: | Amino Acid Sequence |
| TTM-001 | 981 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, starts | | NLGRAVFQAKKRLLEPLGLVEEAAKTAPGKKRPVEQSPQEPDSSAGIGKSGAQPAK |
| at position 456 | | KRLNFGQTGDTESVPDPQPIGEPPAAPSGVGSLTMASGGGAPVADNNEGADGVGSS |
| (immediately | | SGNWHCDSQWLGDRVITTSTRTWALPTYNNHLYKQISNSTSGGSSNDNAYFGYSTP |
| subsequent to | | WGYFDFNRFHCHFSPRDWQRLINNNWGFRPKRLNFKLFNIQVKEVTDNNGVKTIAN |
| position 455); | | NLTSTVQVFTDSDYQLPYVLGSAHEGCLPPFPADVFMIPQYGYLTLNDGSQAVGRS |
| 742 aa | | SFYCLEYFPSQMLRTGNNFQFSYEFENVPFHSSYAHSQSLDRLMNPLIDQYLYYLS |
| | | KTINGSGSPHSKAQNQQTLKFSVAGPSNMAVQGRNYIPGPSYRQQRVSTTVTQNNN |
| | | SEFAWPGASSWALNGRNSLMNPGPAMASHKEGEDRFFPLSGSLIFGKQGTGRDNVD |
| | | ADKVMITNEEEIKTTNPVATESYGQVATNHQSAQAQAQTGWVQNQGILPGMVWQDR |
| | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-002 | 982 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, starts | | NLGRAVFQAKKRLLEPLGLVEEAAKTAPGKKRPVEQSPQEPDSSAGIGKSGAQPAK |
| at position 454 | | KRLNFGQTGDTESVPDPQPIGEPPAAPSGVGSLTMASGGGAPVADNNEGADGVGSS |
| (immediately | | SGNWHCDSQWLGDRVITTSTRTWALPTYNNHLYKQISNSTSGGSSNDNAYFGYSTP |
| subsequent to | | WGYFDFNRFHCHFSPRDWQRLINNNWGFRPKRLNFKLFNIQVKEVTDNNGVKTIAN |
| position 453); | | NLTSTVQVFTDSDYQLPYVLGSAHEGCLPPFPADVFMIPQYGYLTLNDGSQAVGRS |
| 742 aa | | SFYCLEYFPSQMLRTGNNFQFSYEFENVPFHSSYAHSQSLDRLMNPLIDQYLYYLS |
| | | KTINGHDSPHKSGQNQQTLKFSVAGPSNMAVQGRNYIPGPSYRQQRVSTTVTQNNN |
| | | SEFAWPGASSWALNGRNSLMNPGPAMASHKEGEDRFFPLSGSLIFGKQGTGRDNVD |
| | | ADKVMITNEEEIKTTNPVATESYGQVATNHQSAQAQAQTGWVQNQGILPGMVWQDR |
| | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-003 | 36 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, starts | | NLGRAVFQAKKRLLEPLGLVEEAAKTAPGKKRPVEQSPQEPDSSAGIGKSGAQPAK |
| at position 456 | | KRLNFGQTGDTESVPDPQPIGEPPAAPSGVGSLTMASGGGAPVADNNEGADGVGSS |
| (immediately | | SGNWHCDSQWLGDRVITTSTRTWALPTYNNHLYKQISNSTSGGSSNDNAYFGYSTP |
| subsequent to | | WGYFDFNRFHCHFSPRDWQRLINNNWGFRPKRLNFKLFNIQVKEVTDNNGVKTIAN |
| position 455); | | NLTSTVQVFTDSDYQLPYVLGSAHEGCLPPFPADVFMIPQYGYLTLNDGSQAVGRS |
| modifications at | | SFYCLEYFPSQMLRTGNNFQFSYEFENVPFHSSYAHSQSLDRLMNPLIDQYLYYLS |
| positions 451, | | KTERVSGSPHSKAQNQQTLKFSVAGPSNMAVQGRNYIPGPSYRQQRVSTTVTQNNN |
| 452, and 453 | | SEFAWPGASSWALNGRNSLMNPGPAMASHKEGEDRFFPLSGSLIFGKQGTGRDNVD |
| underlined; | | ADKVMITNEEEIKTTNPVATESYGQVATNHQSAQAQAQTGWVQNQGILPGMVWQDR |
| 742 aa | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-004 | 37 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, starts | | NLGRAVFQAKKRLLEPLGLVEEAAKTAPGKKRPVEQSPQEPDSSAGIGKSGAQPAK |
| at position 454 | | KRLNFGQTGDTESVPDPQPIGEPPAAPSGVGSLTMASGGGAPVADNNEGADGVGSS |
| (immediately | | SGNWHCDSQWLGDRVITTSTRTWALPTYNNHLYKQISNSTSGGSSNDNAYFGYSTP |
| subsequent to | | WGYFDFNRFHCHFSPRDWQRLINNNWGFRPKRLNFKLFNIQVKEVTDNNGVKTIAN |
| position 453); | | NLTSTVQVFTDSDYQLPYVLGSAHEGCLPPFPADVFMIPQYGYLTLNDGSQAVGRS |
| modifications at | | SFYCLEYFPSQMLRTGNNFQFSYEFENVPFHSSYAHSQSLDRLMNPLIDQYLYYLS |
| positions 450, | | KAEIGHDSPHKSGQNQQTLKFSVAGPSNMAVQGRNYIPGPSYRQQRVSTTVTQNNN |
| 451, and 452 | | SEFAWPGASSWALNGRNSLMNPGPAMASHKEGEDRFFPLSGSLIFGKQGTGRDNVD |
| underlined; | | ADKVMITNEEEIKTTNPVATESYGQVATNHQSAQAQAQTGWVQNQGILPGMVWQDR |
| 742 aa | | DVYLQGPIWAKIPHTDGNFHPSPLMGGFGMKHPPPQILIKNTPVPADPPTAFNKDK |
| | | LNSLITQYSTGQVSVEIEWELQKENSKRWNPEIQYTSNYYKSNNVEFAVNTEGVYS |
| | | EPRPIGTRYLTRNL |
| TTM-005 | 38 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, starts | | NLGRAVFQAKKRLLEPLGLVEEAAKTAPGKKRPVEQSPQEPDSSAGIGKSGAQPAK |
| at position 456 | | KRLNFGQTGDTESVPDPQPIGEPPAAPSGVGSLTMASGGGAPVADNNEGADGVGSS |
| (immediately | | SGNWHCDSQWLGDRVITTSTRTWALPTYNNHLYKQISNSTSGGSSNDNAYFGYSTP |
| subsequent to | | WGYFDFNRFHCHFSPRDWQRLINNNWGFRPKRLNFKLFNIQVKEVTDNNGVKTIAN |
| position 455); | | NLTSTVQVFTDSDYQLPYVLGSAHEGCLPPFPADVFMIPQYGYLTLNDGSQAVGRS |
| modifications at | | SFYCLEYFPSQMLRTGNNFQFSYEFENVPFHSSYAHSQSLDRLMNPLIDQYLYYLS |
| positions 452, | | KTIIGSGSPHSKAQNRHTLKFSVAGPSNMAVQGRNYIPGPSYRQQRVSTTVTQNNN |
| 464, and 465 | | SEFAWPGASSWALNGRNSLMNPGPAMASHKEGEDRFFPLSGSLIFGKQGTGRDNVD |
| underlined; | | ADKVMITNEEEIKTTNPVATESYGQVATNHQSAQAQAQTGWVQNQGILPGMVWQDR |
| 742 aa | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-006 | 39 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, starts | | NLGRAVFQAKKRLLEPLGLVEEAAKTAPGKKRPVEQSPQEPDSSAGIGKSGAQPAK |
| at position 456 | | KRLNFGQTGDTESVPDPQPIGEPPAAPSGVGSLTMASGGGAPVADNNEGADGVGSS |
| (immediately | | SGNWHCDSQWLGDRVITTSTRTWALPTYNNHLYKQISNSTSGGSSNDNAYFGYSTP |
| subsequent to | | WGYFDFNRFHCHFSPRDWQRLINNNWGFRPKRLNFKLFNIQVKEVTDNNGVKTIAN |
| position 455); | | NLTSTVQVFTDSDYQLPYVLGSAHEGCLPPFPADVFMIPQYGYLTLNDGSQAVGRS |
| modifications at | | SFYCLEYFPSQMLRTGNNFQFSYEFENVPFHSSYAHSQSLDRLMNPLIDQYLYYLS |
| positions 451, | | KTEKMSGSPHSKAQNQQTLKFSVAGPSNMAVQGRNYIPGPSYRQQRVSTTVTQNNN |
| 452, and 453 | | SEFAWPGASSWALNGRNSLMNPGPAMASHKEGEDRFFPLSGSLIFGKQGTGRDNVD |
| underlined; | | ADKVMITNEEEIKTTNPVATESYGQVATNHQSAQAQAQTGWVQNQGILPGMVWQDR |
| 742 aa | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-007 | 40 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, starts | | NLGRAVFQAKKRLLEPLGLVEEAAKTAPGKKRPVEQSPQEPDSSAGIGKSGAQPAK |
| at position 456 | | KRLNFGQTGDTESVPDPQPIGEPPAAPSGVGSLTMASGGGAPVADNNEGADGVGSS |
| (immediately | | SGNWHCDSQWLGDRVITTSTRTWALPTYNNHLYKQISNSTSGGSSNDNAYFGYSTP |
| subsequent to | | WGYFDFNRFHCHFSPRDWQRLINNNWGFRPKRLNFKLFNIQVKEVTDNNGVKTIAN |
| position 455); | | NLTSTVQVFTDSDYQLPYVLGSAHEGCLPPFPADVFMIPQYGYLTLNDGSQAVGRS |
| modifications at | | SFYCLEYFPSQMLRTGNNFQFSYEFENVPFHSSYAHSQSLDRLMNPLIDQYLYYLS |
| positions 450 | | KEINGRGSPHSKAQNQQTLKFSVAGPSNMAVQGRNYIPGPSYRQQRVSTTVTQNNN |
| and 454 | | SEFAWPGASSWALNGRNSLMNPGPAMASHKEGEDRFFPLSGSLIFGKQGTGRDNVD |
| underlined; | | ADKVMITNEEEIKTTNPVATESYGQVATNHQSAQAQAQTGWVQNQGILPGMVWQDR |
| 742 aa | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-008 | 41 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, starts | | NLGRAVFQAKKRLLEPLGLVEEAAKTAPGKKRPVEQSPQEPDSSAGIGKSGAQPAK |
| at position 456 | | KRLNFGQTGDTESVPDPQPIGEPPAAPSGVGSLTMASGGGAPVADNNEGADGVGSS |
| (immediately | | SGNWHCDSQWLGDRVITTSTRTWALPTYNNHLYKQISNSTSGGSSNDNAYFGYSTP |
| subsequent to | | WGYFDFNRFHCHFSPRDWQRLINNNWGFRPKRLNFKLFNIQVKEVTDNNGVKTIAN |
| position 455); | | NLTSTVQVFTDSDYQLPYVLGSAHEGCLPPFPADVFMIPQYGYLTLNDGSQAVGRS |
| modifications at | | SFYCLEYFPSQMLRTGNNFQFSYEFENVPFHSSYAHSQSLDRLMNPLIDQYLYYLS |
| positions 451, | | KTFNGSGSPHSKAPNLQTLKFSVAGPSNMAVQGRNYIPGPSYRQQRVSTTVTQNNN |
| 462, and 464 | | SEFAWPGASSWALNGRNSLMNPGPAMASHKEGEDRFFPLSGSLIFGKQGTGRDNVD |
| underlined; | | ADKVMITNEEEIKTTNPVATESYGQVATNHQSAQAQAQTGWVQNQGILPGMVWQDR |
| 742 aa | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-009 | 42 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, starts | | NLGRAVFQAKKRLLEPLGLVEEAAKTAPGKKRPVEQSPQEPDSSAGIGKSGAQPAK |
| at position 456 | | KRLNFGQTGDTESVPDPQPIGEPPAAPSGVGSLTMASGGGAPVADNNEGADGVGSS |
| (immediately | | SGNWHCDSQWLGDRVITTSTRTWALPTYNNHLYKQISNSTSGGSSNDNAYFGYSTP |
| subsequent to | | WGYFDFNRFHCHFSPRDWQRLINNNWGFRPKRLNFKLFNIQVKEVTDNNGVKTIAN |
| position 455); | | NLTSTVQVFTDSDYQLPYVLGSAHEGCLPPFPADVFMIPQYGYLTLNDGSQAVGRS |
| modifications at | | SFYCLEYFPSQMLRTGNNFQFSYEFENVPFHSSYAHSQSLDRLMNPLIDQYLYYLS |
| positions 451, | | KTEKTSGSPHSKAQNQQTLKFSVAGPSNMAVQGRNYIPGPSYRQQRVSTTVTQNNN |
| 452, and 453 | | SEFAWPGASSWALNGRNSLMNPGPAMASHKEGEDRFFPLSGSLIFGKQGTGRDNVD |
| underlined; | | ADKVMITNEEEIKTTNPVATESYGQVATNHQSAQAQAQTGWVQNQGILPGMVWQDR |
| 742 aa | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-010 | 43 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, starts | | NLGRAVFQAKKRLLEPLGLVEEAAKTAPGKKRPVEQSPQEPDSSAGIGKSGAQPAK |
| at position 456 | | KRLNFGQTGDTESVPDPQPIGEPPAAPSGVGSLTMASGGGAPVADNNEGADGVGSS |
| (immediately | | SGNWHCDSQWLGDRVITTSTRTWALPTYNNHLYKQISNSTSGGSSNDNAYFGYSTP |
| subsequent to | | WGYFDFNRFHCHFSPRDWQRLINNNWGFRPKRLNFKLFNIQVKEVTDNNGVKTIAN |
| position 455); | | NLTSTVQVFTDSDYQLPYVLGSAHEGCLPPFPADVFMIPQYGYLTLNDGSQAVGRS |
| modifications at | | SFYCLEYFPSQMLRTGNNFQFSYEFENVPFHSSYAHSQSLDRLMNPLIDQYLYYLS |
| positions 451, | | KTMNGHDSPHSKAQNQQTLKFSVAGPSNMAVQGRNYIPGPSYRQQRVSTTVTQNNN |
| 454, and 455 | | SEFAWPGASSWALNGRNSLMNPGPAMASHKEGEDRFFPLSGSLIFGKQGTGRDNVD |
| underlined; | | ADKVMITNEEEIKTTNPVATESYGQVATNHQSAQAQAQTGWVQNQGILPGMVWQDR |
| 742 aa | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-011 | 44 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, starts | | NLGRAVFQAKKRLLEPLGLVEEAAKTAPGKKRPVEQSPQEPDSSAGIGKSGAQPAK |
| at position 456 | | KRLNFGQTGDTESVPDPQPIGEPPAAPSGVGSLTMASGGGAPVADNNEGADGVGSS |
| (immediately | | SGNWHCDSQWLGDRVITTSTRTWALPTYNNHLYKQISNSTSGGSSNDNAYFGYSTP |
| subsequent to | | WGYFDFNRFHCHFSPRDWQRLINNNWGFRPKRLNFKLFNIQVKEVTDNNGVKTIAN |
| position 455); | | NLTSTVQVFTDSDYQLPYVLGSAHEGCLPPFPADVFMIPQYGYLTLNDGSQAVGRS |
| modifications at | | SFYCLEYFPSQMLRTGNNFQFSYEFENVPFHSSYAHSQSLDRLMNPLIDQYLYYLS |
| positions 452, | | KTIDGHDSPHSKAQNQQTLKFSVAGPSNMAVQGRNYIPGPSYRQQRVSTTVTQNNN |
| 454, and 455 | | SEFAWPGASSWALNGRNSLMNPGPAMASHKEGEDRFFPLSGSLIFGKQGTGRDNVD |
| underlined; | | ADKVMITNEEEIKTTNPVATESYGQVATNHQSAQAQAQTGWVQNQGILPGMVWQDR |
| 742 aa | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-012 | 45 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, starts | | NLGRAVFQAKKRLLEPLGLVEEAAKTAPGKKRPVEQSPQEPDSSAGIGKSGAQPAK |
| at position 456 | | KRLNFGQTGDTESVPDPQPIGEPPAAPSGVGSLTMASGGGAPVADNNEGADGVGSS |
| (immediately | | SGNWHCDSQWLGDRVITTSTRTWALPTYNNHLYKQISNSTSGGSSNDNAYFGYSTP |
| subsequent to | | WGYFDFNRFHCHFSPRDWQRLINNNWGFRPKRLNFKLFNIQVKEVTDNNGVKTIAN |
| position 455); | | NLTSTVQVFTDSDYQLPYVLGSAHEGCLPPFPADVFMIPQYGYLTLNDGSQAVGRS |
| modifications at | | SFYCLEYFPSQMLRTGNNFQFSYEFENVPFHSSYAHSQSLDRLMNPLIDQYLYYLS |
| positions 452, | | KTNNGHDSPHSKAQNQQTLKFSVAGPSNMAVQGRNYIPGPSYRQQRVSTTVTQNNN |
| 454, and 455 | | SEFAWPGASSWALNGRNSLMNPGPAMASHKEGEDRFFPLSGSLIFGKQGTGRDNVD |
| underlined; | | ADKVMITNEEEIKTTNPVATESYGQVATNHQSAQAQAQTGWVQNQGILPGMVWQDR |
| 742 aa | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-013 | 46 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, starts | | NLGRAVFQAKKRLLEPLGLVEEAAKTAPGKKRPVEQSPQEPDSSAGIGKSGAQPAK |
| at position 456 | | KRLNFGQTGDTESVPDPQPIGEPPAAPSGVGSLTMASGGGAPVADNNEGADGVGSS |
| (immediately | | SGNWHCDSQWLGDRVITTSTRTWALPTYNNHLYKQISNSTSGGSSNDNAYFGYSTP |
| subsequent to | | WGYFDFNRFHCHFSPRDWQRLINNNWGFRPKRLNFKLFNIQVKEVTDNNGVKTIAN |
| position 455); | | NLTSTVQVFTDSDYQLPYVLGSAHEGCLPPFPADVFMIPQYGYLTLNDGSQAVGRS |
| modifications at | | SFYCLEYFPSQMLRTGNNFQFSYEFENVPFHSSYAHSQSLDRLMNPLIDQYLYYLS |
| positions 451, | | KTQRKSGSPHSKAQNQQTLKFSVAGPSNMAVQGRNYIPGPSYRQQRVSTTVTQNNN |
| 452, and 453 | | SEFAWPGASSWALNGRNSLMNPGPAMASHKEGEDRFFPLSGSLIFGKQGTGRDNVD |
| underlined; | | ADKVMITNEEEIKTTNPVATESYGQVATNHQSAQAQAQTGWVQNQGILPGMVWQDR |
| 742 aa | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-014 | 47 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, starts | | NLGRAVFQAKKRLLEPLGLVEEAAKTAPGKKRPVEQSPQEPDSSAGIGKSGAQPAK |
| at position 456 | | KRLNFGQTGDTESVPDPQPIGEPPAAPSGVGSLTMASGGGAPVADNNEGADGVGSS |
| (immediately | | SGNWHCDSQWLGDRVITTSTRTWALPTYNNHLYKQISNSTSGGSSNDNAYFGYSTP |
| subsequent to | | WGYFDFNRFHCHFSPRDWQRLINNNWGFRPKRLNFKLFNIQVKEVTDNNGVKTIAN |
| position 455); | | NLTSTVQVFTDSDYQLPYVLGSAHEGCLPPFPADVFMIPQYGYLTLNDGSQAVGRS |
| modifications at | | SFYCLEYFPSQMLRTGNNFQFSYEFENVPFHSSYAHSQSLDRLMNPLIDQYLYYLS |
| positions 463, | | KTINGSGSPHSKAQARKTLKFSVAGPSNMAVQGRNYIPGPSYRQQRVSTTVTQNNN |
| 464, and 465 | | SEFAWPGASSWALNGRNSLMNPGPAMASHKEGEDRFFPLSGSLIFGKQGTGRDNVD |
| underlined; | | ADKVMITNEEEIKTTNPVATESYGQVATNHQSAQAQAQTGWVQNQGILPGMVWQDR |
| 742 aa | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-015 | 48 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, starts | | NLGRAVFQAKKRLLEPLGLVEEAAKTAPGKKRPVEQSPQEPDSSAGIGKSGAQPAK |
| at position 456 | | KRLNFGQTGDTESVPDPQPIGEPPAAPSGVGSLTMASGGGAPVADNNEGADGVGSS |
| (immediately | | SGNWHCDSQWLGDRVITTSTRTWALPTYNNHLYKQISNSTSGGSSNDNAYFGYSTP |
| subsequent to | | WGYFDFNRFHCHFSPRDWQRLINNNWGFRPKRLNFKLFNIQVKEVTDNNGVKTIAN |
| position 455); | | NLTSTVQVFTDSDYQLPYVLGSAHEGCLPPFPADVFMIPQYGYLTLNDGSQAVGRS |
| modifications at | | SFYCLEYFPSQMLRTGNNFQFSYEFENVPFHSSYAHSQSLDRLMNPLIDQYLYYLS |
| positions 450 | | KYIVGSGSPHSKAQNQQTLKFSVAGPSNMAVQGRNYIPGPSYRQQRVSTTVTQNNN |
| and 452 | | SEFAWPGASSWALNGRNSLMNPGPAMASHKEGEDRFFPLSGSLIFGKQGTGRDNVD |
| underlined; | | ADKVMITNEEEIKTTNPVATESYGQVATNHQSAQAQAQTGWVQNQGILPGMVWQDR |
| 742 aa | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-016 | 49 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, starts | | NLGRAVFQAKKRLLEPLGLVEEAAKTAPGKKRPVEQSPQEPDSSAGIGKSGAQPAK |
| at position 456 | | KRLNFGQTGDTESVPDPQPIGEPPAAPSGVGSLTMASGGGAPVADNNEGADGVGSS |
| (immediately | | SGNWHCDSQWLGDRVITTSTRTWALPTYNNHLYKQISNSTSGGSSNDNAYFGYSTP |
| subsequent to | | WGYFDFNRFHCHFSPRDWQRLINNNWGFRPKRLNFKLFNIQVKEVTDNNGVKTIAN |
| position 455); | | NLTSTVQVFTDSDYQLPYVLGSAHEGCLPPFPADVFMIPQYGYLTLNDGSQAVGRS |
| modifications at | | SFYCLEYFPSQMLRTGNNFQFSYEFENVPFHSSYAHSQSLDRLMNPLIDQYLYYLS |
| positions 452, | | KTISKRGSPHSKAQNQQTLKFSVAGPSNMAVQGRNYIPGPSYRQQRVSTTVTQNNN |
| 453, and 454 | | SEFAWPGASSWALNGRNSLMNPGPAMASHKEGEDRFFPLSGSLIFGKQGTGRDNVD |
| underlined; | | ADKVMITNEEEIKTTNPVATESYGQVATNHQSAQAQAQTGWVQNQGILPGMVWQDR |
| 742 aa | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-017 | 50 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, starts | | NLGRAVFQAKKRLLEPLGLVEEAAKTAPGKKRPVEQSPQEPDSSAGIGKSGAQPAK |
| at position 456 | | KRLNFGQTGDTESVPDPQPIGEPPAAPSGVGSLTMASGGGAPVADNNEGADGVGSS |
| (immediately | | SGNWHCDSQWLGDRVITTSTRTWALPTYNNHLYKQISNSTSGGSSNDNAYFGYSTP |
| subsequent to | | WGYFDFNRFHCHFSPRDWQRLINNNWGFRPKRLNFKLFNIQVKEVTDNNGVKTIAN |
| position 455); | | NLTSTVQVFTDSDYQLPYVLGSAHEGCLPPFPADVFMIPQYGYLTLNDGSQAVGRS |
| modifications at | | SFYCLEYFPSQMLRTGNNFQFSYEFENVPFHSSYAHSQSLDRLMNPLIDQYLYYLS |
| positions 450, | | KGLGGSGSPHSKAQNQQTLKFSVAGPSNMAVQGRNYIPGPSYRQQRVSTTVTQNNN |
| 451, and 452 | | SEFAWPGASSWALNGRNSLMNPGPAMASHKEGEDRFFPLSGSLIFGKQGTGRDNVD |
| underlined; | | ADKVMITNEEEIKTTNPVATESYGQVATNHQSAQAQAQTGWVQNQGILPGMVWQDR |
| 742 aa | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-018 | 51 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, starts | | NLGRAVFQAKKRLLEPLGLVEEAAKTAPGKKRPVEQSPQEPDSSAGIGKSGAQPAK |
| at position 456 | | KRLNFGQTGDTESVPDPQPIGEPPAAPSGVGSLTMASGGGAPVADNNEGADGVGSS |
| (immediately | | SGNWHCDSQWLGDRVITTSTRTWALPTYNNHLYKQISNSTSGGSSNDNAYFGYSTP |
| subsequent to | | WGYFDFNRFHCHFSPRDWQRLINNNWGFRPKRLNFKLFNIQVKEVTDNNGVKTIAN |
| position 455); | | NLTSTVQVFTDSDYQLPYVLGSAHEGCLPPFPADVFMIPQYGYLTLNDGSQAVGRS |
| modifications at | | SFYCLEYFPSQMLRTGNNFQFSYEFENVPFHSSYAHSQSLDRLMNPLIDQYLYYLS |
| positions 454, | | KTINGHDSPHSKAQNLQTLKFSVAGPSNMAVQGRNYIPGPSYRQQRVSTTVTQNNN |
| 455, and 464 | | SEFAWPGASSWALNGRNSLMNPGPAMASHKEGEDRFFPLSGSLIFGKQGTGRDNVD |
| underlined; | | ADKVMITNEEEIKTTNPVATESYGQVATNHQSAQAQAQTGWVQNQGILPGMVWQDR |
| 742 aa | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-019 | 52 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, starts | | NLGRAVFQAKKRLLEPLGLVEEAAKTAPGKKRPVEQSPQEPDSSAGIGKSGAQPAK |
| at position 456 | | KRLNFGQTGDTESVPDPQPIGEPPAAPSGVGSLTMASGGGAPVADNNEGADGVGSS |
| (immediately | | SGNWHCDSQWLGDRVITTSTRTWALPTYNNHLYKQISNSTSGGSSNDNAYFGYSTP |
| subsequent to | | WGYFDFNRFHCHFSPRDWQRLINNNWGFRPKRLNFKLFNIQVKEVTDNNGVKTIAN |
| position 455); | | NLTSTVQVFTDSDYQLPYVLGSAHEGCLPPFPADVFMIPQYGYLTLNDGSQAVGRS |
| modifications at | | SFYCLEYFPSQMLRTGNNFQFSYEFENVPFHSSYAHSQSLDRLMNPLIDQYLYYLS |
| positions 451, | | KTVNGHDSPHSKAQNQQTLKFSVAGPSNMAVQGRNYIPGPSYRQQRVSTTVTQNNN |
| 454, and 455 | | SEFAWPGASSWALNGRNSLMNPGPAMASHKEGEDRFFPLSGSLIFGKQGTGRDNVD |
| underlined; | | ADKVMITNEEEIKTTNPVATESYGQVATNHQSAQAQAQTGWVQNQGILPGMVWQDR |
| 742 aa | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-020 | 53 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, starts | | NLGRAVFQAKKRLLEPLGLVEEAAKTAPGKKRPVEQSPQEPDSSAGIGKSGAQPAK |
| at position 456 | | KRLNFGQTGDTESVPDPQPIGEPPAAPSGVGSLTMASGGGAPVADNNEGADGVGSS |
| (immediately | | SGNWHCDSQWLGDRVITTSTRTWALPTYNNHLYKQISNSTSGGSSNDNAYFGYSTP |
| subsequent to | | WGYFDFNRFHCHFSPRDWQRLINNNWGFRPKRLNFKLFNIQVKEVTDNNGVKTIAN |
| position 455); | | NLTSTVQVFTDSDYQLPYVLGSAHEGCLPPFPADVFMIPQYGYLTLNDGSQAVGRS |
| modifications at | | SFYCLEYFPSQMLRTGNNFQFSYEFENVPFHSSYAHSQSLDRLMNPLIDQYLYYLS |
| positions 454, | | KTINGHDSPHSKALNQQTLKFSVAGPSNMAVQGRNYIPGPSYRQQRVSTTVTQNNN |
| 455, and 462 | | SEFAWPGASSWALNGRNSLMNPGPAMASHKEGEDRFFPLSGSLIFGKQGTGRDNVD |
| underlined; | | ADKVMITNEEEIKTTNPVATESYGQVATNHQSAQAQAQTGWVQNQGILPGMVWQDR |
| 742 aa | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-021 | 54 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, starts | | NLGRAVFQAKKRLLEPLGLVEEAAKTAPGKKRPVEQSPQEPDSSAGIGKSGAQPAK |
| at position 456 | | KRLNFGQTGDTESVPDPQPIGEPPAAPSGVGSLTMASGGGAPVADNNEGADGVGSS |
| (immediately | | SGNWHCDSQWLGDRVITTSTRTWALPTYNNHLYKQISNSTSGGSSNDNAYFGYSTP |
| subsequent to | | WGYFDFNRFHCHFSPRDWQRLINNNWGFRPKRLNFKLFNIQVKEVTDNNGVKTIAN |
| position 455); | | NLTSTVQVFTDSDYQLPYVLGSAHEGCLPPFPADVFMIPQYGYLTLNDGSQAVGRS |
| modifications at | | SFYCLEYFPSQMLRTGNNFQFSYEFENVPFHSSYAHSQSLDRLMNPLIDQYLYYLS |
| positions 454, | | KTINGHDSPHSKAQNQQSLKFSVAGPSNMAVQGRNYIPGPSYRQQRVSTTVTQNNN |
| 455, and 466 | | SEFAWPGASSWALNGRNSLMNPGPAMASHKEGEDRFFPLSGSLIFGKQGTGRDNVD |
| underlined; | | ADKVMITNEEEIKTTNPVATESYGQVATNHQSAQAQAQTGWVQNQGILPGMVWQDR |
| 742 aa | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-022 | 55 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, starts | | NLGRAVFQAKKRLLEPLGLVEEAAKTAPGKKRPVEQSPQEPDSSAGIGKSGAQPAK |
| at position 456 | | KRLNFGQTGDTESVPDPQPIGEPPAAPSGVGSLTMASGGGAPVADNNEGADGVGSS |
| (immediately | | SGNWHCDSQWLGDRVITTSTRTWALPTYNNHLYKQISNSTSGGSSNDNAYFGYSTP |
| subsequent to | | WGYFDFNRFHCHFSPRDWQRLINNNWGFRPKRLNFKLFNIQVKEVTDNNGVKTIAN |
| position 455); | | NLTSTVQVFTDSDYQLPYVLGSAHEGCLPPFPADVFMIPQYGYLTLNDGSQAVGRS |
| modifications at | | SFYCLEYFPSQMLRTGNNFQFSYEFENVPFHSSYAHSQSLDRLMNPLIDQYLYYLS |
| positions 454, | | KTINGHDSPHSKAQNQQILKFSVAGPSNMAVQGRNYIPGPSYRQQRVSTTVTQNNN |
| 455, and 466 | | SEFAWPGASSWALNGRNSLMNPGPAMASHKEGEDRFFPLSGSLIFGKQGTGRDNVD |
| underlined; | | ADKVMITNEEEIKTTNPVATESYGQVATNHQSAQAQAQTGWVQNQGILPGMVWQDR |
| 742 aa | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-023 | 56 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, starts | | NLGRAVFQAKKRLLEPLGLVEEAAKTAPGKKRPVEQSPQEPDSSAGIGKSGAQPAK |
| at position 456 | | KRLNFGQTGDTESVPDPQPIGEPPAAPSGVGSLTMASGGGAPVADNNEGADGVGSS |
| (immediately | | SGNWHCDSQWLGDRVITTSTRTWALPTYNNHLYKQISNSTSGGSSNDNAYFGYSTP |
| subsequent to | | WGYFDFNRFHCHFSPRDWQRLINNNWGFRPKRLNFKLFNIQVKEVTDNNGVKTIAN |
| position 455); | | NLTSTVQVFTDSDYQLPYVLGSAHEGCLPPFPADVFMIPQYGYLTLNDGSQAVGRS |
| 742 aa | | SFYCLEYFPSQMLRTGNNFQFSYEFENVPFHSSYAHSQSLDRLMNPLIDQYLYYLS |
| | | KTINGSGSPHFTRQNQQTLKFSVAGPSNMAVQGRNYIPGPSYRQQRVSTTVTQNNN |
| | | SEFAWPGASSWALNGRNSLMNPGPAMASHKEGEDRFFPLSGSLIFGKQGTGRDNVD |
| | | ADKVMITNEEEIKTTNPVATESYGQVATNHQSAQAQAQTGWVQNQGILPGMVWQDR |
| | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-024 | 57 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, starts | | NLGRAVFQAKKRLLEPLGLVEEAAKTAPGKKRPVEQSPQEPDSSAGIGKSGAQPAK |
| at position 456 | | KRLNFGQTGDTESVPDPQPIGEPPAAPSGVGSLTMASGGGAPVADNNEGADGVGSS |
| (immediately | | SGNWHCDSQWLGDRVITTSTRTWALPTYNNHLYKQISNSTSGGSSNDNAYFGYSTP |
| subsequent to | | WGYFDFNRFHCHFSPRDWQRLINNNWGFRPKRLNFKLFNIQVKEVTDNNGVKTIAN |
| position 455); | | NLTSTVQVFTDSDYQLPYVLGSAHEGCLPPFPADVFMIPQYGYLTLNDGSQAVGRS |
| modifications at | | SFYCLEYFPSQMLRTGNNFQFSYEFENVPFHSSYAHSQSLDRLMNPLIDQYLYYLS |
| positions 451, | | KTSNGHDSPHSKAQNQQTLKFSVAGPSNMAVQGRNYIPGPSYRQQRVSTTVTQNNN |
| 454, and 455 | | SEFAWPGASSWALNGRNSLMNPGPAMASHKEGEDRFFPLSGSLIFGKQGTGRDNVD |
| underlined; | | ADKVMITNEEEIKTTNPVATESYGQVATNHQSAQAQAQTGWVQNQGILPGMVWQDR |
| 742 aa | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-025 | 58 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, starts | | NLGRAVFQAKKRLLEPLGLVEEAAKTAPGKKRPVEQSPQEPDSSAGIGKSGAQPAK |
| at position 456 | | KRLNFGQTGDTESVPDPQPIGEPPAAPSGVGSLTMASGGGAPVADNNEGADGVGSS |
| (immediately | | SGNWHCDSQWLGDRVITTSTRTWALPTYNNHLYKQISNSTSGGSSNDNAYFGYSTP |
| subsequent to | | WGYFDFNRFHCHFSPRDWQRLINNNWGFRPKRLNFKLFNIQVKEVTDNNGVKTIAN |
| position 455); | | NLTSTVQVFTDSDYQLPYVLGSAHEGCLPPFPADVFMIPQYGYLTLNDGSQAVGRS |
| modification at | | SFYCLEYFPSQMLRTGNNFQFSYEFENVPFHSSYAHSQSLDRLMNPLIDQYLYYLS |
| position 462 | | KTINGSGSPHSLPWNQQTLKFSVAGPSNMAVQGRNYIPGPSYRQQRVSTTVTQNNN |
| underlined; | | SEFAWPGASSWALNGRNSLMNPGPAMASHKEGEDRFFPLSGSLIFGKQGTGRDNVD |
| 742 aa | | ADKVMITNEEEIKTTNPVATESYGQVATNHQSAQAQAQTGWVQNQGILPGMVWQDR |
| | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-026 | 59 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, starts | | NLGRAVFQAKKRLLEPLGLVEEAAKTAPGKKRPVEQSPQEPDSSAGIGKSGAQPAK |
| at position 456 | | KRLNFGQTGDTESVPDPQPIGEPPAAPSGVGSLTMASGGGAPVADNNEGADGVGSS |
| (immediately | | SGNWHCDSQWLGDRVITTSTRTWALPTYNNHLYKQISNSTSGGSSNDNAYFGYSTP |
| subsequent to | | WGYFDFNRFHCHFSPRDWQRLINNNWGFRPKRLNFKLFNIQVKEVTDNNGVKTIAN |
| position 455); | | NLTSTVQVFTDSDYQLPYVLGSAHEGCLPPFPADVFMIPQYGYLTLNDGSQAVGRS |
| modification at | | SFYCLEYFPSQMLRTGNNFQFSYEFENVPFHSSYAHSQSLDRLMNPLIDQYLYYLS |
| positions 454, | | KTINGHDSPHSKAQNHQTLKFSVAGPSNMAVQGRNYIPGPSYRQQRVSTTVTQNNN |
| 455, and 464 | | SEFAWPGASSWALNGRNSLMNPGPAMASHKEGEDRFFPLSGSLIFGKQGTGRDNVD |
| underlined; | | ADKVMITNEEEIKTTNPVATESYGQVATNHQSAQAQAQTGWVQNQGILPGMVWQDR |
| 742 aa | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-027 | 69 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, starts | | NLGRAVFQAKKRLLEPLGLVEEAAKTAPGKKRPVEQSPQEPDSSAGIGKSGAQPAK |
| at position 456 | | KRLNFGQTGDTESVPDPQPIGEPPAAPSGVGSLTMASGGGAPVADNNEGADGVGSS |
| (immediately | | SGNWHCDSQWLGDRVITTSTRTWALPTYNNHLYKQISNSTSGGSSNDNAYFGYSTP |
| subsequent to | | WGYFDFNRFHCHFSPRDWQRLINNNWGFRPKRLNFKLFNIQVKEVTDNNGVKTIAN |
| position 455); | | NLTSTVQVFTDSDYQLPYVLGSAHEGCLPPFPADVFMIPQYGYLTLNDGSQAVGRS |
| modification at | | SFYCLEYFPSQMLRTGNNFQFSYEFENVPFHSSYAHSQSLDRLMNPLIDQYLYYLS |
| positions 451 | | KTENVSGSPHSKAQNQQTLKFSVAGPSNMAVQGRNYIPGPSYRQQRVSTTVTQNNN |
| and 453 | | SEFAWPGASSWALNGRNSLMNPGPAMASHKEGEDRFFPLSGSLIFGKQGTGRDNVD |
| underlined; | | ADKVMITNEEEIKTTNPVATESYGQVATNHQSAQAQAQTGWVQNQGILPGMVWQDR |
| 742 aa | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-028 | 70 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, starts | | NLGRAVFQAKKRLLEPLGLVEEAAKTAPGKKRPVEQSPQEPDSSAGIGKSGAQPAK |
| at position 456 | | KRLNFGQTGDTESVPDPQPIGEPPAAPSGVGSLTMASGGGAPVADNNEGADGVGSS |
| (immediately | | SGNWHCDSQWLGDRVITTSTRTWALPTYNNHLYKQISNSTSGGSSNDNAYFGYSTP |
| subsequent to | | WGYFDFNRFHCHFSPRDWQRLINNNWGFRPKRLNFKLFNIQVKEVTDNNGVKTIAN |
| position 455); | | NLTSTVQVFTDSDYQLPYVLGSAHEGCLPPFPADVFMIPQYGYLTLNDGSQAVGRS |
| modification at | | SFYCLEYFPSQMLRTGNNFQFSYEFENVPFHSSYAHSQSLDRLMNPLIDQYLYYLS |
| positions 451, | | KTEKVSGSPHSKAQNQQTLKFSVAGPSNMAVQGRNYIPGPSYRQQRVSTTVTQNNN |
| 452, and 453 | | SEFAWPGASSWALNGRNSLMNPGPAMASHKEGEDRFFPLSGSLIFGKQGTGRDNVD |
| underlined; | | ADKVMITNEEEIKTTNPVATESYGQVATNHQSAQAQAQTGWVQNQGILPGMVWQDR |
| 742 aa | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-029 | 71 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, starts | | NLGRAVFQAKKRLLEPLGLVEEAAKTAPGKKRPVEQSPQEPDSSAGIGKSGAQPAK |
| at position 456 | | KRLNFGQTGDTESVPDPQPIGEPPAAPSGVGSLTMASGGGAPVADNNEGADGVGSS |
| (immediately | | SGNWHCDSQWLGDRVITTSTRTWALPTYNNHLYKQISNSTSGGSSNDNAYFGYSTP |
| subsequent to | | WGYFDFNRFHCHFSPRDWQRLINNNWGFRPKRLNFKLFNIQVKEVTDNNGVKTIAN |
| position 455); | | NLTSTVQVFTDSDYQLPYVLGSAHEGCLPPFPADVFMIPQYGYLTLNDGSQAVGRS |
| modification at | | SFYCLEYFPSQMLRTGNNFQFSYEFENVPFHSSYAHSQSLDRLMNPLIDQYLYYLS |
| positions 464, | | KTINGSGSPHKSGQNKTSLKFSVAGPSNMAVQGRNYIPGPSYRQQRVSTTVTQNNN |
| 465, and 466 | | SEFAWPGASSWALNGRNSLMNPGPAMASHKEGEDRFFPLSGSLIFGKQGTGRDNVD |
| underlined; | | ADKVMITNEEEIKTTNPVATESYGQVATNHQSAQAQAQTGWVQNQGILPGMVWQDR |
| 742 aa | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-030 | 4 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, starts | | NLGRAVFQAKKRLLEPLGLVEEAAKTAPGKKRPVEQSPQEPDSSAGIGKSGAQPAK |
| at position 456 | | KRLNFGQTGDTESVPDPQPIGEPPAAPSGVGSLTMASGGGAPVADNNEGADGVGSS |
| (immediately | | SGNWHCDSQWLGDRVITTSTRTWALPTYNNHLYKQISNSTSGGSSNDNAYFGYSTP |
| subsequent to | | WGYFDFNRFHCHFSPRDWQRLINNNWGFRPKRLNFKLFNIQVKEVTDNNGVKTIAN |
| position 455); | | NLTSTVQVFTDSDYQLPYVLGSAHEGCLPPFPADVFMIPQYGYLTLNDGSQAVGRS |
| modification at | | SFYCLEYFPSQMLRTGNNFQFSYEFENVPFHSSYAHSQSLDRLMNPLIDQYLYYLS |
| positions 451, | | KTSNASGSPHSKAHNQQTLKFSVAGPSNMAVQGRNYIPGPSYRQQRVSTTVTQNNN |
| 453, and 462 | | SEFAWPGASSWALNGRNSLMNPGPAMASHKEGEDRFFPLSGSLIFGKQGTGRDNVD |
| underlined; | | ADKVMITNEEEIKTTNPVATESYGQVATNHQSAQAQAQTGWVQNQGILPGMVWQDR |
| 742 aa | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-031 | 5 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, starts | | NLGRAVFQAKKRLLEPLGLVEEAAKTAPGKKRPVEQSPQEPDSSAGIGKSGAQPAK |
| at position 456 | | KRLNFGQTGDTESVPDPQPIGEPPAAPSGVGSLTMASGGGAPVADNNEGADGVGSS |
| (immediately | | SGNWHCDSQWLGDRVITTSTRTWALPTYNNHLYKQISNSTSGGSSNDNAYFGYSTP |
| subsequent to | | WGYFDFNRFHCHFSPRDWQRLINNNWGFRPKRLNFKLFNIQVKEVTDNNGVKTIAN |
| position 455); | | NLTSTVQVFTDSDYQLPYVLGSAHEGCLPPFPADVFMIPQYGYLTLNDGSQAVGRS |
| modification at | | SFYCLEYFPSQMLRTGNNFQFSYEFENVPFHSSYAHSQSLDRLMNPLIDQYLYYLS |
| positions 450, | | KNSIGSGSPHSKAQNQQTLKFSVAGPSNMAVQGRNYIPGPSYRQQRVSTTVTQNNN |
| 451, and 452 | | SEFAWPGASSWALNGRNSLMNPGPAMASHKEGEDRFFPLSGSLIFGKQGTGRDNVD |
| underlined; | | ADKVMITNEEEIKTTNPVATESYGQVATNHQSAQAQAQTGWVQNQGILPGMVWQDR |
| 742 aa | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-032 | 6 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, starts | | NLGRAVFQAKKRLLEPLGLVEEAAKTAPGKKRPVEQSPQEPDSSAGIGKSGAQPAK |
| at position 454 | | KRLNFGQTGDTESVPDPQPIGEPPAAPSGVGSLTMASGGGAPVADNNEGADGVGSS |
| (immediately | | SGNWHCDSQWLGDRVITTSTRTWALPTYNNHLYKQISNSTSGGSSNDNAYFGYSTP |
| subsequent to | | WGYFDFNRFHCHFSPRDWQRLINNNWGFRPKRLNFKLFNIQVKEVTDNNGVKTIAN |
| position 453); | | NLTSTVQVFTDSDYQLPYVLGSAHEGCLPPFPADVFMIPQYGYLTLNDGSQAVGRS |
| modification at | | SFYCLEYFPSQMLRTGNNFQFSYEFENVPFHSSYAHSQSLDRLMNPLIDQYLYYLS |
| positions 451, | | KTVNGHDSPHKSGQRPSTLKFSVAGPSNMAVQGRNYIPGPSYRQQRVSTTVTQNNN |
| 463, 464, and | | SEFAWPGASSWALNGRNSLMNPGPAMASHKEGEDRFFPLSGSLIFGKQGTGRDNVD |
| 465 underlined; | | ADKVMITNEEEIKTTNPVATESYGQVATNHQSAQAQAQTGWVQNQGILPGMVWQDR |
| 742 aa | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-033 | 7 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, starts | | NLGRAVFQAKKRLLEPLGLVEEAAKTAPGKKRPVEQSPQEPDSSAGIGKSGAQPAK |
| at position 456 | | KRLNFGQTGDTESVPDPQPIGEPPAAPSGVGSLTMASGGGAPVADNNEGADGVGSS |
| (immediately | | SGNWHCDSQWLGDRVITTSTRTWALPTYNNHLYKQISNSTSGGSSNDNAYFGYSTP |
| subsequent to | | WGYFDFNRFHCHFSPRDWQRLINNNWGFRPKRLNFKLFNIQVKEVTDNNGVKTIAN |
| position 455); | | NLTSTVQVFTDSDYQLPYVLGSAHEGCLPPFPADVFMIPQYGYLTLNDGSQAVGRS |
| modification at | | SFYCLEYFPSQMLRTGNNFQFSYEFENVPFHSSYAHSQSLDRLMNPLIDQYLYYLS |
| positions 450 | | KAENGSGSPHSKAQNQQTLKFSVAGPSNMAVQGRNYIPGPSYRQQRVSTTVTQNNN |
| and 451 | | SEFAWPGASSWALNGRNSLMNPGPAMASHKEGEDRFFPLSGSLIFGKQGTGRDNVD |
| underlined; | | ADKVMITNEEEIKTTNPVATESYGQVATNHQSAQAQAQTGWVQNQGILPGMVWQDR |
| 742 aa | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-034 | 8 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, starts | | NLGRAVFQAKKRLLEPLGLVEEAAKTAPGKKRPVEQSPQEPDSSAGIGKSGAQPAK |
| at position 456 | | KRLNFGQTGDTESVPDPQPIGEPPAAPSGVGSLTMASGGGAPVADNNEGADGVGSS |
| (immediately | | SGNWHCDSQWLGDRVITTSTRTWALPTYNNHLYKQISNSTSGGSSNDNAYFGYSTP |
| subsequent to | | WGYFDFNRFHCHFSPRDWQRLINNNWGFRPKRLNFKLFNIQVKEVTDNNGVKTIAN |
| position 455); | | NLTSTVQVFTDSDYQLPYVLGSAHEGCLPPFPADVFMIPQYGYLTLNDGSQAVGRS |
| modification at | | SFYCLEYFPSQMLRTGNNFQFSYEFENVPFHSSYAHSQSLDRLMNPLIDQYLYYLS |
| position 466 | | KTINGSGSPHKSGQNQQPLKFSVAGPSNMAVQGRNYIPGPSYRQQRVSTTVTQNNN |
| underlined; | | SEFAWPGASSWALNGRNSLMNPGPAMASHKEGEDRFFPLSGSLIFGKQGTGRDNVD |
| 742 aa | | ADKVMITNEEEIKTTNPVATESYGQVATNHQSAQAQAQTGWVQNQGILPGMVWQDR |
| | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |

TABLE 5
Exemplary full length capsid nucleic acid sequences

| Name and | SEQ ID | |
|---|---|---|
| Annotation | NO: | NT Sequence |
| TTM-001 | 983 | ATGGCTGCCGATGGTTATCTTCCAGATTGGCTCGAGGACAACCTTAGTGAAGGAATTCG |
| 9mer | | CGAGTGGTGGGCTTTGAAACCTGGAGCCCCTCAACCCAAGGCAAATCAACAACATCAAG |
| peptide | | ACAACGCTCGAGGTCTTGTGCTTCCGGGTTACAAATACCTTGGACCCGGCAACGGACTC |
| underlined | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGIGGTGGCGCACCAGIGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGACCAATACTTGTATTACTTGAGTAAAACAATTAACG |
| | | GAAGCGGAAGCCCACACAGCAAAGCACAAAACCAACAGACCTTGAAGTTTTCGGTAGCT |
| | | GGTCCTAGCAACATGGCTGTCCAGGGAAGAAACTACATACCTGGACCCAGCTACCGACA |
| | | ACAACGTGTCTCAACCACTGTGACTCAAAACAACAACAGCGAATTTGCTTGGCCTGGAG |
| | | CTTCTTCTTGGGCTCTCAATGGACGTAATAGCTTGATGAATCCTGGACCTGCTATGGCC |
| | | AGCCACAAAGAAGGAGAGGACCGTTTCTTTCCTTTGTCTGGATCTTTAATTTTTGGCAA |
| | | ACAAGGAACTGGAAGAGACAACGTGGATGCGGACAAAGTCATGATAACCAACGAAGAAG |
| | | AAATTAAAACTACTAACCCGGTAGCAACGGAGTCCTATGGACAAGTGGCCACAAACCAC |
| | | CAGAGTGCCCAAGCACAGGCGCAGACCGGCTGGGTTCAAAACCAAGGAATACTTCCGGG |
| | | TATGGTTTGGCAGGACAGAGATGTGTACCTGCAAGGACCCATTTGGGCCAAAATTCCTC |
| | | ACACGGACGGCAACTTTCACCCTTCTCCGCTGATGGGAGGGTTTGGAATGAAGCACCCG |
| | | CCTCCTCAGATCCTCATCAAAAACACACCTGTACCTGCGGATCCTCCAACGGCCTTCAA |
| | | CAAGGACAAGCTGAACTCTTTCATCACCCAGTATTCTACTGGCCAAGTCAGCGTGGAGA |
| | | TCGAGTGGGAGCTGCAGAAGGAAAACAGCAAGCGCTGGAACCCGGAGATCCAGTACACT |
| | | TCCAACTATTACAAGTCTAATAATGTTGAATTTGCTGTTAATACTGAAGGTGTATATAG |
| | | TGAACCCCGCCCCATTGGCACGCGGTATTTAACGAGGAACTTATAA |
| TTM-002 | 984 | ATGGCTGCCGATGGTTATCTTCCAGATTGGCTCGAGGACAACCTTAGTGAAGGAATTCG |
| 7mer | | CGAGTGGTGGGCTTTGAAACCTGGAGCCCCTCAACCCAAGGCAAATCAACAACATCAAG |
| peptide | | ACAACGCTCGAGGTCTTGTGCTTCCGGGTTACAAATACCTTGGACCCGGCAACGGACTC |
| underlined | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCITAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGACCAATACTTGTATTACTTGAGTAAAACAATTAACG |
| | | GACACGACAGCCCACACAAAAGCGGACAAAACCAACAGACCTTGAAGTTTTCGGTAGCT |
| | | GGTCCTAGCAACATGGCTGTCCAGGGAAGAAACTACATACCTGGACCCAGCTACCGACA |
| | | ACAACGTGTCTCAACCACTGTGACTCAAAACAACAACAGCGAATTTGCTTGGCCTGGAG |
| | | CTTCTTCTTGGGCTCTCAATGGACGTAATAGCTTGATGAATCCTGGACCTGCTATGGCC |
| | | AGCCACAAAGAAGGAGAGGACCGTTTCTTTCCTTTGTCTGGATCTTTAATTTTTGGCAA |
| | | ACAAGGAACTGGAAGAGACAACGTGGATGCGGACAAAGTCATGATAACCAACGAAGAAG |
| | | AAATTAAAACTACTAACCCGGTAGCAACGGAGTCCTATGGACAAGTGGCCACAAACCAC |
| | | CAGAGTGCCCAAGCACAGGCGCAGACCGGCTGGGTTCAAAACCAAGGAATACTTCCGGG |
| | | TATGGTTTGGCAGGACAGAGATGTGTACCTGCAAGGACCCATTTGGGCCAAAATTCCTC |
| | | ACACGGACGGCAACTTTCACCCTTCTCCGCTGATGGGAGGGTTTGGAATGAAGCACCCG |
| | | CCTCCTCAGATCCTCATCAAAAACACACCTGTACCTGCGGATCCTCCAACGGCCTTCAA |
| | | CAAGGACAAGCTGAACTCTTTCATCACCCAGTATTCTACTGGCCAAGTCAGCGTGGAGA |
| | | TCGAGTGGGAGCTGCAGAAGGAAAACAGCAAGCGCTGGAACCCGGAGATCCAGTACACT |
| | | TCCAACTATTACAAGTCTAATAATGTTGAATTTGCTGTTAATACTGAAGGTGTATATAG |
| | | TGAACCCCGCCCCATTGGCACGCGGTATTTAACGAGGAACTTATAA |
| TTM-003 | 72 | ATGGCTGCCGATGGTTATCTTCCAGATTGGCTCGAGGACAACCTTAGTGAAGGAATTCG |
| | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCIGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGATCAGTATCTGTATTACTTGAGTAAGACGGAGCGTG |
| | | TGTCTGGTTCTCCGCATTCTAAGGCGCAGAATCAGCAGACGTTGAAGTTTTCGGTAGCT |
| | | GGTCCTAGCAACATGGCTGTCCAGGGAAGAAACTACATACCTGGACCCAGCTACCGACA |
| | | ACAACGTGTCTCAACCACTGTGACTCAAAACAACAACAGCGAATTTGCTTGGCCTGGAG |
| | | CTTCTTCTTGGGCTCTCAATGGACGTAATAGCTTGATGAATCCTGGACCTGCTATGGCC |
| | | AGCCACAAAGAAGGAGAGGACCGTTTCTTTCCTTTGTCTGGATCTTTAATTTTTGGCAA |
| | | ACAAGGAACTGGAAGAGACAACGTGGATGCGGACAAAGTCATGATAACCAACGAAGAAG |
| | | AAATTAAAACTACTAACCCGGTAGCAACGGAGTCCTATGGACAAGTGGCCACAAACCAC |
| | | CAGAGTGCCCAAGCACAGGCGCAGACCGGCTGGGTTCAAAACCAAGGAATACTTCCGGG |
| | | TATGGTTTGGCAGGACAGAGATGTGTACCTGCAAGGACCCATTTGGGCCAAAATTCCTC |
| | | ACACGGACGGCAACTTTCACCCTTCTCCGCTGATGGGAGGGTTTGGAATGAAGCACCCG |
| | | CCTCCTCAGATCCTCATCAAAAACACACCTGTACCTGCGGATCCTCCAACGGCCTTCAA |
| | | CAAGGACAAGCTGAACTCTTTCATCACCCAGTATTCTACTGGCCAAGTCAGCGTGGAGA |
| | | TCGAGTGGGAGCTGCAGAAGGAAAACAGCAAGCGCTGGAACCCGGAGATCCAGTACACT |
| | | TCCAACTATTACAAGTCTAATAATGTTGAATTTGCTGTTAATACTGAAGGTGTATATAG |
| | | TGAACCCCGCCCCATTGGCACGCGGTATTTAACGAGGAACTTA |
| TTM-004 | 73 | ATGGCTGCCGATGGTTATCTTCCAGATTGGCTCGAGGACAACCTTAGTGAAGGAATTCG |
| | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGATCAGTATCTGTATTACTTGAGTAAGGCGGAGATTG |
| | | GTCATGATTCTCCGCATAAGTCTGGTCAGAATCAGCAGACGTTGAAGTTTTCGGTAGCT |
| | | GGTCCTAGCAACATGGCTGTCCAGGGAAGAAACTACATACCTGGACCCAGCTACCGACA |
| | | ACAACGTGTCTCAACCACTGTGACTCAAAACAACAACAGCGAATTTGCTTGGCCTGGAG |
| | | CTTCTTCTTGGGCTCTCAATGGACGTAATAGCTTGATGAATCCTGGACCTGCTATGGCC |
| | | AGCCACAAAGAAGGAGAGGACCGTTTCTTTCCTTTGTCTGGATCTTTAATTTTTGGCAA |
| | | ACAAGGAACTGGAAGAGACAACGTGGATGCGGACAAAGTCATGATAACCAACGAAGAAG |
| | | AAATTAAAACTACTAACCCGGTAGCAACGGAGTCCTATGGACAAGTGGCCACAAACCAC |
| | | CAGAGTGCCCAAGCACAGGCGCAGACCGGCTGGGTTCAAAACCAAGGAATACTTCCGGG |
| | | TATGGTTTGGCAGGACAGAGATGTGTACCTGCAAGGACCCATTTGGGCCAAAATTCCTC |
| | | ACACGGACGGCAACTTTCACCCTTCTCCGCTGATGGGAGGGTTTGGAATGAAGCACCCG |
| | | CCTCCTCAGATCCTCATCAAAAACACACCTGTACCTGCGGATCCTCCAACGGCCTTCAA |
| | | CAAGGACAAGCTGAACTCTTTAATCACCCAGTATTCTACTGGCCAAGTCAGCGTGGAGA |
| | | TCGAGTGGGAGCTGCAGAAGGAAAACAGCAAGCGCTGGAACCCGGAGATCCAGTACACT |
| | | TCCAACTATTACAAGTCTAATAATGTTGAATTTGCTGTTAATACTGAAGGTGTATATAG |
| | | TGAACCCCGCCCCATTGGCACGCGGTATTTAACGAGGAACTTA |
| TTM-005 | 74 | ATGGCTGCCGATGGTTATCTTCCAGATTGGCTCGAGGACAACCTTAGTGAAGGAATTCG |
| | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGATCAGTATCTGTATTACTTGAGTAAGACGATTATTG |
| | | GTTCTGGTTCTCCGCATTCTAAGGCGCAGAATCGTCATACGTTGAAGTTTTCGGTAGCT |
| | | GGTCCTAGCAACATGGCTGTCCAGGGAAGAAACTACATACCTGGACCCAGCTACCGACA |
| | | ACAACGTGTCTCAACCACTGTGACTCAAAACAACAACAGCGAATTTGCTTGGCCTGGAG |
| | | CTTCTTCTTGGGCTCTCAATGGACGTAATAGCTTGATGAATCCTGGACCTGCTATGGCC |
| | | AGCCACAAAGAAGGAGAGGACCGTTTCTTTCCTTTGTCTGGATCTTTAATTTTTGGCAA |
| | | ACAAGGAACTGGAAGAGACAACGTGGATGCGGACAAAGTCATGATAACCAACGAAGAAG |
| | | AAATTAAAACTACTAACCCGGTAGCAACGGAGTCCTATGGACAAGTGGCCACAAACCAC |
| | | CAGAGTGCCCAAGCACAGGCGCAGACCGGCTGGGTTCAAAACCAAGGAATACTTCCGGG |
| | | TATGGTTTGGCAGGACAGAGATGTGTACCTGCAAGGACCCATTTGGGCCAAAATTCCTC |
| | | ACACGGACGGCAACTTTCACCCTTCTCCGCTGATGGGAGGGTTTGGAATGAAGCACCCG |
| | | CCTCCTCAGATCCTCATCAAAAACACACCTGTACCTGCGGATCCTCCAACGGCCTTCAA |
| | | CAAGGACAAGCTGAACTCTTTCATCACCCAGTATTCTACTGGCCAAGTCAGCGTGGAGA |
| | | TCGAGTGGGAGCTGCAGAAGGAAAACAGCAAGCGCTGGAACCCGGAGATCCAGTACACT |
| | | TCCAACTATTACAAGTCTAATAATGTTGAATTTGCTGTTAATACTGAAGGTGTATATAG |
| | | TGAACCCCGCCCCATTGGCACGCGGTATTTAACGAGGAACTTA |
| TTM-006 | 75 | ATGGCTGCCGATGGTTATCTTCCAGATTGGCTCGAGGACAACCTTAGTGAAGGAATTCG |
| | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGATCAGTATCTGTATTACTTGAGTAAGACGGAGAAGA |
| | | TGTCTGGTTCTCCGCATTCTAAGGCGCAGAATCAGCAGACGTTGAAGTTTTCGGTAGCT |
| | | GGTCCTAGCAACATGGCTGTCCAGGGAAGAAACTACATACCTGGACCCAGCTACCGACA |
| | | ACAACGTGTCTCAACCACTGTGACTCAAAACAACAACAGCGAATTTGCTTGGCCTGGAG |
| | | CTTCTTCTTGGGCTCTCAATGGACGTAATAGCTTGATGAATCCTGGACCTGCTATGGCC |
| | | AGCCACAAAGAAGGAGAGGACCGTTTCTTTCCTTTGTCTGGATCTTTAATTTTTGGCAA |
| | | ACAAGGAACTGGAAGAGACAACGTGGATGCGGACAAAGTCATGATAACCAACGAAGAAG |
| | | AAATTAAAACTACTAACCCGGTAGCAACGGAGTCCTATGGACAAGTGGCCACAAACCAC |
| | | CAGAGTGCCCAAGCACAGGCGCAGACCGGCTGGGTTCAAAACCAAGGAATACTTCCGGG |
| | | TATGGTTTGGCAGGACAGAGATGTGTACCTGCAAGGACCCATTTGGGCCAAAATTCCTC |
| | | ACACGGACGGCAACTTTCACCCTTCTCCGCTGATGGGAGGGTTTGGAATGAAGCACCCG |
| | | CCTCCTCAGATCCTCATCAAAAACACACCTGTACCTGCGGATCCTCCAACGGCCTTCAA |
| | | CAAGGACAAGCTGAACTCTTTCATCACCCAGTATTCTACTGGCCAAGTCAGCGTGGAGA |
| | | TCGAGTGGGAGCTGCAGAAGGAAAACAGCAAGCGCTGGAACCCGGAGATCCAGTACACT |
| | | TCCAACTATTACAAGTCTAATAATGTTGAATTTGCTGTTAATACTGAAGGTGTATATAG |
| | | TGAACCCCGCCCCATTGGCACGCGGTATTTAACGAGGAACTTA |
| TTM-007 | 76 | ATGGCTGCCGATGGTTATCTTCCAGATTGGCTCGAGGACAACCTTAGTGAAGGAATTCG |
| | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGATCAGTATCTGTATTACTTGAGTAAGGAGATTAATG |
| | | GTCGTGGTTCTCCGCATTCTAAGGCGCAGAATCAGCAGACGTTGAAGTTTTCGGTAGCT |
| | | GGTCCTAGCAACATGGCTGTCCAGGGAAGAAACTACATACCTGGACCCAGCTACCGACA |
| | | ACAACGTGTCTCAACCACTGTGACTCAAAACAACAACAGCGAATTTGCTTGGCCTGGAG |
| | | CTTCTTCTTGGGCTCTCAATGGACGTAATAGCTTGATGAATCCTGGACCTGCTATGGCC |
| | | AGCCACAAAGAAGGAGAGGACCGTTTCTTTCCTTTGTCTGGATCTTTAATTTTTGGCAA |
| | | ACAAGGAACTGGAAGAGACAACGTGGATGCGGACAAAGTCATGATAACCAACGAAGAAG |
| | | AAATTAAAACTACTAACCCGGTAGCAACGGAGTCCTATGGACAAGTGGCCACAAACCAC |
| | | CAGAGTGCCCAAGCACAGGCGCAGACCGGCTGGGTTCAAAACCAAGGAATACTTCCGGG |
| | | TATGGTTTGGCAGGACAGAGATGTGTACCTGCAAGGACCCATTTGGGCCAAAATTCCTC |
| | | ACACGGACGGCAACTTTCACCCTTCTCCGCTGATGGGAGGGTTTGGAATGAAGCACCCG |
| | | CCTCCTCAGATCCTCATCAAAAACACACCTGTACCTGCGGATCCTCCAACGGCCTTCAA |
| | | CAAGGACAAGCTGAACTCTTTCATCACCCAGTATTCTACTGGCCAAGTCAGCGTGGAGA |
| | | TCGAGTGGGAGCTGCAGAAGGAAAACAGCAAGCGCTGGAACCCGGAGATCCAGTACACT |
| | | TCCAACTATTACAAGTCTAATAATGTTGAATTTGCTGTTAATACTGAAGGTGTATATAG |
| | | TGAACCCCGCCCCATTGGCACGCGGTATTTAACGAGGAACTTA |
| TTM-008 | 77 | ATGGCTGCCGATGGTTATCTTCCAGATTGGCTCGAGGACAACCTTAGTGAAGGAATTCG |
| | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGATCAGTATCTGTATTACTTGAGTAAGACGTTTAATG |
| | | GTTCTGGTTCTCCGCATTCTAAGGCGCCGAATCTGCAGACGTTGAAGTTTTCGGTAGCT |
| | | GGTCCTAGCAACATGGCTGTCCAGGGAAGAAACTACATACCTGGACCCAGCTACCGACA |
| | | ACAACGTGTCTCAACCACTGTGACTCAAAACAACAACAGCGAATTTGCTTGGCCTGGAG |
| | | CTTCTTCTTGGGCTCTCAATGGACGTAATAGCTTGATGAATCCTGGACCTGCTATGGCC |
| | | AGCCACAAAGAAGGAGAGGACCGTTTCTTTCCTTTGTCTGGATCTTTAATTTTTGGCAA |
| | | ACAAGGAACTGGAAGAGACAACGTGGATGCGGACAAAGTCATGATAACCAACGAAGAAG |
| | | AAATTAAAACTACTAACCCGGTAGCAACGGAGTCCTATGGACAAGTGGCCACAAACCAC |
| | | CAGAGTGCCCAAGCACAGGCGCAGACCGGCTGGGTTCAAAACCAAGGAATACTTCCGGG |
| | | TATGGTTTGGCAGGACAGAGATGTGTACCTGCAAGGACCCATTTGGGCCAAAATTCCTC |
| | | ACACGGACGGCAACTTTCACCCTTCTCCGCTGATGGGAGGGTTTGGAATGAAGCACCCG |
| | | CCTCCTCAGATCCTCATCAAAAACACACCTGTACCTGCGGATCCTCCAACGGCCTTCAA |
| | | CAAGGACAAGCTGAACTCTTTCATCACCCAGTATTCTACTGGCCAAGTCAGCGTGGAGA |
| | | TCGAGTGGGAGCTGCAGAAGGAAAACAGCAAGCGCTGGAACCCGGAGATCCAGTACACT |
| | | TCCAACTATTACAAGTCTAATAATGTTGAATTTGCTGTTAATACTGAAGGTGTATATAG |
| | | TGAACCCCGCCCCATTGGCACGCGGTATTTAACGAGGAACTTA |
| TTM-009 | 78 | ATGGCTGCCGATGGTTATCTTCCAGATTGGCTCGAGGACAACCTTAGTGAAGGAATTCG |
| | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGATCAGTATCTGTATTACTTGAGTAAGACGGAGAAGA |
| | | CGTCTGGTTCTCCGCATTCTAAGGCGCAGAATCAGCAGACGTTGAAGTTTTCGGTAGCT |
| | | GGTCCTAGCAACATGGCTGTCCAGGGAAGAAACTACATACCTGGACCCAGCTACCGACA |
| | | ACAACGTGTCTCAACCACTGTGACTCAAAACAACAACAGCGAATTTGCTTGGCCTGGAG |
| | | CTTCTTCTTGGGCTCTCAATGGACGTAATAGCTTGATGAATCCTGGACCTGCTATGGCC |
| | | AGCCACAAAGAAGGAGAGGACCGTTTCTTTCCTTTGTCTGGATCTTTAATTTTTGGCAA |
| | | ACAAGGAACTGGAAGAGACAACGTGGATGCGGACAAAGTCATGATAACCAACGAAGAAG |
| | | AAATTAAAACTACTAACCCGGTAGCAACGGAGTCCTATGGACAAGTGGCCACAAACCAC |
| | | CAGAGTGCCCAAGCACAGGCGCAGACCGGCTGGGTTCAAAACCAAGGAATACTTCCGGG |
| | | TATGGTTTGGCAGGACAGAGATGTGTACCTGCAAGGACCCATTTGGGCCAAAATTCCTC |
| | | ACACGGACGGCAACTTTCACCCTTCTCCGCTGATGGGAGGGTTTGGAATGAAGCACCCG |
| | | CCTCCTCAGATCCTCATCAAAAACACACCTGTACCTGCGGATCCTCCAACGGCCTTCAA |
| | | CAAGGACAAGCTGAACTCTTTCATCACCCAGTATTCTACTGGCCAAGTCAGCGTGGAGA |
| | | TCGAGTGGGAGCTGCAGAAGGAAAACAGCAAGCGCTGGAACCCGGAGATCCAGTACACT |
| | | TCCAACTATTACAAGTCTAATAATGTTGAATTTGCTGTTAATACTGAAGGTGTATATAG |
| | | TGAACCCCGCCCCATTGGCACGCGGTATTTAACGAGGAACTTA |
| TTM-010 | 79 | ATGGCTGCCGATGGTTATCTTCCAGATTGGCTCGAGGACAACCTTAGTGAAGGAATTCG |
| | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGATCAGTATCTGTATTACTTGAGTAAGACGATGAATG |
| | | GTCATGATTCTCCGCATTCTAAGGCGCAGAATCAGCAGACGTTGAAGTTTTCGGTAGCT |
| | | GGTCCTAGCAACATGGCTGTCCAGGGAAGAAACTACATACCTGGACCCAGCTACCGACA |
| | | ACAACGTGTCTCAACCACTGTGACTCAAAACAACAACAGCGAATTTGCTTGGCCTGGAG |
| | | CTTCTTCTTGGGCTCTCAATGGACGTAATAGCTTGATGAATCCTGGACCTGCTATGGCC |
| | | AGCCACAAAGAAGGAGAGGACCGTTTCTTTCCTTTGTCTGGATCTTTAATTTTTGGCAA |
| | | ACAAGGAACTGGAAGAGACAACGTGGATGCGGACAAAGTCATGATAACCAACGAAGAAG |
| | | AAATTAAAACTACTAACCCGGTAGCAACGGAGTCCTATGGACAAGTGGCCACAAACCAC |
| | | CAGAGTGCCCAAGCACAGGCGCAGACCGGCTGGGTTCAAAACCAAGGAATACTTCCGGG |
| | | TATGGTTTGGCAGGACAGAGATGTGTACCTGCAAGGACCCATTTGGGCCAAAATTCCTC |
| | | ACACGGACGGCAACTTTCACCCTTCTCCGCTGATGGGAGGGTTTGGAATGAAGCACCCG |
| | | CCTCCTCAGATCCTCATCAAAAACACACCTGTACCTGCGGATCCTCCAACGGCCTTCAA |
| | | CAAGGACAAGCTGAACTCTTTCATCACCCAGTATTCTACTGGCCAAGTCAGCGTGGAGA |
| | | TCGAGTGGGAGCTGCAGAAGGAAAACAGCAAGCGCTGGAACCCGGAGATCCAGTACACT |
| | | TCCAACTATTACAAGTCTAATAATGTTGAATTTGCTGTTAATACTGAAGGTGTATATAG |
| | | TGAACCCCGCCCCATTGGCACGCGGTATTTAACGAGGAACTTA |
| TTM-011 | 80 | ATGGCTGCCGATGGTTATCTTCCAGATTGGCTCGAGGACAACCTTAGTGAAGGAATTCG |
| | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGATCAGTATCTGTATTACTTGAGTAAGACGATTGATG |
| | | GTCATGATTCTCCGCATTCTAAGGCGCAGAATCAGCAGACGTTGAAGTTTTCGGTAGCT |
| | | GGTCCTAGCAACATGGCTGTCCAGGGAAGAAACTACATACCTGGACCCAGCTACCGACA |
| | | ACAACGTGTCTCAACCACTGIGACTCAAAACAACAACAGCGAATTTGCTTGGCCTGGAG |
| | | CTTCTTCTTGGGCTCTCAATGGACGTAATAGCTTGATGAATCCTGGACCTGCTATGGCC |
| | | AGCCACAAAGAAGGAGAGGACCGTTTCTTTCCTTTGTCTGGATCTTTAATTTTTGGCAA |
| | | ACAAGGAACTGGAAGAGACAACGTGGATGCGGACAAAGTCATGATAACCAACGAAGAAG |
| | | AAATTAAAACTACTAACCCGGTAGCAACGGAGTCCTATGGACAAGTGGCCACAAACCAC |
| | | CAGAGTGCCCAAGCACAGGCGCAGACCGGCTGGGTTCAAAACCAAGGAATACTTCCGGG |
| | | TATGGTTTGGCAGGACAGAGATGTGTACCTGCAAGGACCCATTTGGGCCAAAATTCCTC |
| | | ACACGGACGGCAACTTTCACCCTTCTCCGCTGATGGGAGGGTTTGGAATGAAGCACCCG |
| | | CCTCCTCAGATCCTCATCAAAAACACACCTGTACCTGCGGATCCTCCAACGGCCTTCAA |
| | | CAAGGACAAGCTGAACTCTTTCATCACCCAGTATTCTACTGGCCAAGTCAGCGTGGAGA |
| | | TCGAGTGGGAGCTGCAGAAGGAAAACAGCAAGCGCTGGAACCCGGAGATCCAGTACACT |
| | | TCCAACTATTACAAGTCTAATAATGTTGAATTTGCTGTTAATACTGAAGGTGTATATAG |
| | | TGAACCCCGCCCCATTGGCACGCGGTATTTAACGAGGAACTTA |
| TTM-012 | 81 | ATGGCTGCCGATGGTTATCTTCCAGATTGGCTCGAGGACAACCTTAGTGAAGGAATTCG |
| | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGIGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGATCAGTATCTGTATTACTTGAGTAAGACGAATAATG |
| | | GTCATGATTCTCCGCATTCTAAGGCGCAGAATCAGCAGACGTTGAAGTTTTCGGTAGCT |
| | | GGTCCTAGCAACATGGCTGTCCAGGGAAGAAACTACATACCTGGACCCAGCTACCGACA |
| | | ACAACGTGTCTCAACCACTGTGACTCAAAACAACAACAGCGAATTTGCTTGGCCTGGAG |
| | | CTTCTTCTTGGGCTCTCAATGGACGTAATAGCTTGATGAATCCTGGACCTGCTATGGCC |
| | | AGCCACAAAGAAGGAGAGGACCGTTTCTTTCCTTTGTCTGGATCTTTAATTTTTGGCAA |
| | | ACAAGGAACTGGAAGAGACAACGTGGATGCGGACAAAGTCATGATAACCAACGAAGAAG |
| | | AAATTAAAACTACTAACCCGGTAGCAACGGAGTCCTATGGACAAGTGGCCACAAACCAC |
| | | CAGAGTGCCCAAGCACAGGCGCAGACCGGCTGGGTTCAAAACCAAGGAATACTTCCGGG |
| | | TATGGTTTGGCAGGACAGAGATGTGTACCTGCAAGGACCCATTTGGGCCAAAATTCCTC |
| | | ACACGGACGGCAACTTTCACCCTTCTCCGCTGATGGGAGGGTTTGGAATGAAGCACCCG |
| | | CCTCCTCAGATCCTCATCAAAAACACACCTGTACCTGCGGATCCTCCAACGGCCTTCAA |
| | | CAAGGACAAGCTGAACTCTTTCATCACCCAGTATTCTACTGGCCAAGTCAGCGTGGAGA |
| | | TCGAGTGGGAGCTGCAGAAGGAAAACAGCAAGCGCTGGAACCCGGAGATCCAGTACACT |
| | | TCCAACTATTACAAGTCTAATAATGTTGAATTTGCTGTTAATACTGAAGGTGTATATAG |
| | | TGAACCCCGCCCCATTGGCACGCGGTATTTAACGAGGAACTTA |
| TTM-013 | 22 | ATGGCTGCCGATGGTTATCTTCCAGATTGGCTCGAGGACAACCTTAGTGAAGGAATTCG |
| | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGIGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGATCAGTATCTGTATTACTTGAGTAAGACGCAGCGTA |
| | | AGTCTGGTTCTCCGCATTCTAAGGCGCAGAATCAGCAGACGTTGAAGTTTTCGGTAGCT |
| | | GGTCCTAGCAACATGGCTGTCCAGGGAAGAAACTACATACCTGGACCCAGCTACCGACA |
| | | ACAACGTGTCTCAACCACTGTGACTCAAAACAACAACAGCGAATTTGCTTGGCCTGGAG |
| | | CTTCTTCTTGGGCTCTCAATGGACGTAATAGCTTGATGAATCCTGGACCTGCTATGGCC |
| | | AGCCACAAAGAAGGAGAGGACCGTTTCTTTCCTTTGTCTGGATCTTTAATTTTTGGCAA |
| | | ACAAGGAACTGGAAGAGACAACGTGGATGCGGACAAAGTCATGATAACCAACGAAGAAG |
| | | AAATTAAAACTACTAACCCGGTAGCAACGGAGTCCTATGGACAAGTGGCCACAAACCAC |
| | | CAGAGTGCCCAAGCACAGGCGCAGACCGGCTGGGTTCAAAACCAAGGAATACTTCCGGG |
| | | TATGGTTTGGCAGGACAGAGATGTGTACCTGCAAGGACCCATTTGGGCCAAAATTCCTC |
| | | ACACGGACGGCAACTTTCACCCTTCTCCGCTGATGGGAGGGTTTGGAATGAAGCACCCG |
| | | CCTCCTCAGATCCTCATCAAAAACACACCTGTACCTGCGGATCCTCCAACGGCCTTCAA |
| | | CAAGGACAAGCTGAACTCTTTCATCACCCAGTATTCTACTGGCCAAGTCAGCGTGGAGA |
| | | TCGAGTGGGAGCTGCAGAAGGAAAACAGCAAGCGCTGGAACCCGGAGATCCAGTACACT |
| | | TCCAACTATTACAAGTCTAATAATGTTGAATTTGCTGTTAATACTGAAGGTGTATATAG |
| | | TGAACCCCGCCCCATTGGCACGCGGTATTTAACGAGGAACTTA |
| TTM-014 | 23 | ATGGCTGCCGATGGTTATCTTCCAGATTGGCTCGAGGACAACCTTAGTGAAGGAATTCG |
| | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCIGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGATCAGTATCTGTATTACTTGAGTAAGACGATTAATG |
| | | GTTCTGGTTCTCCGCATTCTAAGGCGCAGGCGCGTAAGACGTTGAAGTTTTCGGTAGCT |
| | | ACAACGTGTCTCAACCACTGTGACTCAAAACAACAACAGCGAATTTGCTTGGCCTGGAG |
| | | GGTCCTAGCAACATGGCTGTCCAGGGAAGAAACTACATACCTGGACCCAGCTACCGACA |
| | | ACAACGTGTCTCAACCACTGTGACTCAAAACAACAACAGCGAATTTGCTTGGCCTGGAG |
| | | CTTCTTCTTGGGCTCTCAATGGACGTAATAGCTTGATGAATCCTGGACCTGCTATGGCC |
| | | AGCCACAAAGAAGGAGAGGACCGTTTCTTTCCTTTGTCTGGATCTTTAATTTTTGGCAA |
| | | ACAAGGAACTGGAAGAGACAACGTGGATGCGGACAAAGTCATGATAACCAACGAAGAAG |
| | | AAATTAAAACTACTAACCCGGTAGCAACGGAGTCCTATGGACAAGTGGCCACAAACCAC |
| | | CAGAGTGCCCAAGCACAGGCGCAGACCGGCTGGGTTCAAAACCAAGGAATACTTCCGGG |
| | | TATGGTTTGGCAGGACAGAGATGTGTACCTGCAAGGACCCATTTGGGCCAAAATTCCTC |
| | | ACACGGACGGCAACTTTCACCCTTCTCCGCTGATGGGAGGGTTTGGAATGAAGCACCCG |
| | | CCTCCTCAGATCCTCATCAAAAACACACCTGTACCTGCGGATCCTCCAACGGCCTTCAA |
| | | CAAGGACAAGCTGAACTCTTTCATCACCCAGTATTCTACTGGCCAAGTCAGCGTGGAGA |
| | | TCGAGTGGGAGCTGCAGAAGGAAAACAGCAAGCGCTGGAACCCGGAGATCCAGTACACT |
| | | TCCAACTATTACAAGTCTAATAATGTTGAATTTGCTGTTAATACTGAAGGTGTATATAG |
| | | TGAACCCCGCCCCATTGGCACGCGGTATTTAACGAGGAACTTA |
| TTM-015 | 24 | ATGGCTGCCGATGGTTATCTTCCAGATTGGCTCGAGGACAACCTTAGTGAAGGAATTCG |
| | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGATCAGTATCTGTATTACTTGAGTAAGTATATTGTGG |
| | | GTTCTGGTTCTCCGCATTCTAAGGCGCAGAATCAGCAGACGTTGAAGTTTTCGGTAGCT |
| | | GGTCCTAGCAACATGGCTGTCCAGGGAAGAAACTACATACCTGGACCCAGCTACCGACA |
| | | ACAACGTGTCTCAACCACTGTGACTCAAAACAACAACAGCGAATTTGCTTGGCCTGGAG |
| | | CTTCTTCTTGGGCTCTCAATGGACGTAATAGCTTGATGAATCCTGGACCTGCTATGGCC |
| | | AGCCACAAAGAAGGAGAGGACCGTTTCTTTCCTTTGTCTGGATCTTTAATTTTTGGCAA |
| | | ACAAGGAACTGGAAGAGACAACGTGGATGCGGACAAAGTCATGATAACCAACGAAGAAG |
| | | AAATTAAAACTACTAACCCGGTAGCAACGGAGTCCTATGGACAAGTGGCCACAAACCAC |
| | | CAGAGTGCCCAAGCACAGGCGCAGACCGGCTGGGTTCAAAACCAAGGAATACTTCCGGG |
| | | TATGGTTTGGCAGGACAGAGATGTGTACCTGCAAGGACCCATTTGGGCCAAAATTCCTC |
| | | ACACGGACGGCAACTTTCACCCTTCTCCGCTGATGGGAGGGTTTGGAATGAAGCACCCG |
| | | CCTCCTCAGATCCTCATCAAAAACACACCTGTACCTGCGGATCCTCCAACGGCCTTCAA |
| | | CAAGGACAAGCTGAACTCTTTCATCACCCAGTATTCTACTGGCCAAGTCAGCGTGGAGA |
| | | TCGAGTGGGAGCTGCAGAAGGAAAACAGCAAGCGCTGGAACCCGGAGATCCAGTACACT |
| | | TCCAACTATTACAAGTCTAATAATGTTGAATTTGCTGTTAATACTGAAGGTGTATATAG |
| | | TGAACCCCGCCCCATTGGCACGCGGTATTTAACGAGGAACTTA |
| TTM-016 | 25 | ATGGCTGCCGATGGTTATCTTCCAGATTGGCTCGAGGACAACCTTAGTGAAGGAATTCG |
| | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGATCAGTATCTGTATTACTTGAGTAAGACGATTTCTA |
| | | AGCGTGGTTCTCCGCATTCTAAGGCGCAGAATCAGCAGACGTTGAAGTTTTCGGTAGCT |
| | | GGTCCTAGCAACATGGCTGTCCAGGGAAGAAACTACATACCTGGACCCAGCTACCGACA |
| | | ACAACGTGTCTCAACCACTGTGACTCAAAACAACAACAGCGAATTTGCTTGGCCTGGAG |
| | | CTTCTTCTTGGGCTCTCAATGGACGTAATAGCTTGATGAATCCTGGACCTGCTATGGCC |
| | | AGCCACAAAGAAGGAGAGGACCGTTTCTTTCCTTTGTCTGGATCTTTAATTTTTGGCAA |
| | | ACAAGGAACTGGAAGAGACAACGTGGATGCGGACAAAGTCATGATAACCAACGAAGAAG |
| | | AAATTAAAACTACTAACCCGGTAGCAACGGAGTCCTATGGACAAGTGGCCACAAACCAC |
| | | CAGAGTGCCCAAGCACAGGCGCAGACCGGCTGGGTTCAAAACCAAGGAATACTTCCGGG |
| | | TATGGTTTGGCAGGACAGAGATGTGTACCTGCAAGGACCCATTTGGGCCAAAATTCCTC |
| | | ACACGGACGGCAACTTTCACCCTTCTCCGCTGATGGGAGGGTTTGGAATGAAGCACCCG |
| | | CCTCCTCAGATCCTCATCAAAAACACACCTGTACCTGCGGATCCTCCAACGGCCTTCAA |
| | | CAAGGACAAGCTGAACTCTTTCATCACCCAGTATTCTACTGGCCAAGTCAGCGTGGAGA |
| | | TCGAGTGGGAGCTGCAGAAGGAAAACAGCAAGCGCTGGAACCCGGAGATCCAGTACACT |
| | | TCCAACTATTACAAGTCTAATAATGTTGAATTTGCTGTTAATACTGAAGGTGTATATAG |
| | | TGAACCCCGCCCCATTGGCACGCGGTATTTAACGAGGAACTTA |
| TTM-017 | 26 | ATGGCTGCCGATGGTTATCTTCCAGATTGGCTCGAGGACAACCTTAGTGAAGGAATTCG |
| | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGATCAGTATCTGTATTACTTGAGTAAGGGTCTGGGTG |
| | | GTTCTGGTTCTCCGCATTCTAAGGCGCAGAATCAGCAGACGTTGAAGTTTTCGGTAGCT |
| | | GGTCCTAGCAACATGGCTGTCCAGGGAAGAAACTACATACCTGGACCCAGCTACCGACA |
| | | ACAACGTGTCTCAACCACTGTGACTCAAAACAACAACAGCGAATTTGCTTGGCCTGGAG |
| | | CTTCTTCTTGGGCTCTCAATGGACGTAATAGCTTGATGAATCCTGGACCTGCTATGGCC |
| | | AGCCACAAAGAAGGAGAGGACCGTTTCTTTCCTTTGTCTGGATCTTTAATTTTTGGCAA |
| | | ACAAGGAACTGGAAGAGACAACGTGGATGCGGACAAAGTCATGATAACCAACGAAGAAG |
| | | AAATTAAAACTACTAACCCGGTAGCAACGGAGTCCTATGGACAAGTGGCCACAAACCAC |
| | | CAGAGTGCCCAAGCACAGGCGCAGACCGGCTGGGTTCAAAACCAAGGAATACTTCCGGG |
| | | TATGGTTTGGCAGGACAGAGATGTGTACCTGCAAGGACCCATTTGGGCCAAAATTCCTC |
| | | ACACGGACGGCAACTTTCACCCTTCTCCGCTGATGGGAGGGTTTGGAATGAAGCACCCG |
| | | CCTCCTCAGATCCTCATCAAAAACACACCTGTACCTGCGGATCCTCCAACGGCCTTCAA |
| | | CAAGGACAAGCTGAACTCTTTCATCACCCAGTATTCTACTGGCCAAGTCAGCGTGGAGA |
| | | TCGAGTGGGAGCTGCAGAAGGAAAACAGCAAGCGCTGGAACCCGGAGATCCAGTACACT |
| | | TCCAACTATTACAAGTCTAATAATGTTGAATTTGCTGTTAATACTGAAGGTGTATATAG |
| | | TGAACCCCGCCCCATTGGCACGCGGTATTTAACGAGGAACTTA |
| TTM-018 | 27 | ATGGCTGCCGATGGTTATCTTCCAGATTGGCTCGAGGACAACCTTAGTGAAGGAATTCG |
| | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTITGAGAACGTACCTTTCCATAGCAGCTACGCTCACAGCCAAAGCCTGGA |
| | | CCGACTAATGAATCCACTCATCGATCAGTATCTGTATTACTTGAGTAAGACGATTAATG |
| | | GTCATGATTCTCCGCATTCTAAGGCGCAGAATCTGCAGACGTTGAAGTTTTCGGTAGCT |
| | | GGTCCTAGCAACATGGCTGTCCAGGGAAGAAACTACATACCTGGACCCAGCTACCGACA |
| | | ACAACGTGTCTCAACCACTGTGACTCAAAACAACAACAGCGAATTTGCTTGGCCTGGAG |
| | | CTTCTTCTTGGGCTCTCAATGGACGTAATAGCTTGATGAATCCTGGACCTGCTATGGCC |
| | | AGCCACAAAGAAGGAGAGGACCGTTTCTTTCCTTTGTCTGGATCTTTAATTTTTGGCAA |
| | | ACAAGGAACTGGAAGAGACAACGTGGATGCGGACAAAGTCATGATAACCAACGAAGAAG |
| | | AAATTAAAACTACTAACCCGGTAGCAACGGAGTCCTATGGACAAGTGGCCACAAACCAC |
| | | CAGAGTGCCCAAGCACAGGCGCAGACCGGCTGGGTTCAAAACCAAGGAATACTTCCGGG |
| | | TATGGTTTGGCAGGACAGAGATGTGTACCTGCAAGGACCCATTTGGGCCAAAATTCCTC |
| | | ACACGGACGGCAACTTTCACCCTTCTCCGCTGATGGGAGGGTTTGGAATGAAGCACCCG |
| | | CCTCCTCAGATCCTCATCAAAAACACACCTGTACCTGCGGATCCTCCAACGGCCTTCAA |
| | | CAAGGACAAGCTGAACTCTTTCATCACCCAGTATTCTACTGGCCAAGTCAGCGTGGAGA |
| | | TCGAGTGGGAGCTGCAGAAGGAAAACAGCAAGCGCTGGAACCCGGAGATCCAGTACACT |
| | | TCCAACTATTACAAGTCTAATAATGTTGAATTTGCTGTTAATACTGAAGGTGTATATAG |
| | | TGAACCCCGCCCCATTGGCACGCGGTATTTAACGAGGAACTTA |
| TTM-019 | 28 | ATGGCTGCCGATGGTTATCTTCCAGATTGGCTCGAGGACAACCTTAGTGAAGGAATTCG |
| | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGIGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGATCAGTATCTGTATTACTTGAGTAAGACGGTGAATG |
| | | GTCATGATTCTCCGCATTCTAAGGCGCAGAATCAGCAGACGTTGAAGTTTTCGGTAGCT |
| | | GGTCCTAGCAACATGGCTGTCCAGGGAAGAAACTACATACCTGGACCCAGCTACCGACA |
| | | ACAACGTGTCTCAACCACTGTGACTCAAAACAACAACAGCGAATTTGCTTGGCCTGGAG |
| | | CTTCTTCTTGGGCTCTCAATGGACGTAATAGCTTGATGAATCCTGGACCTGCTATGGCC |
| | | AGCCACAAAGAAGGAGAGGACCGTTTCTTTCCTTTGTCTGGATCTTTAATTTTTGGCAA |
| | | ACAAGGAACTGGAAGAGACAACGTGGATGCGGACAAAGTCATGATAACCAACGAAGAAG |
| | | AAATTAAAACTACTAACCCGGTAGCAACGGAGTCCTATGGACAAGTGGCCACAAACCAC |
| | | CAGAGTGCCCAAGCACAGGCGCAGACCGGCTGGGTTCAAAACCAAGGAATACTTCCGGG |
| | | TATGGTTTGGCAGGACAGAGATGTGTACCTGCAAGGACCCATTTGGGCCAAAATTCCTC |
| | | ACACGGACGGCAACTTTCACCCTTCTCCGCTGATGGGAGGGTTTGGAATGAAGCACCCG |
| | | CCTCCTCAGATCCTCATCAAAAACACACCTGTACCTGCGGATCCTCCAACGGCCTTCAA |
| | | CAAGGACAAGCTGAACTCTTTCATCACCCAGTATTCTACTGGCCAAGTCAGCGTGGAGA |
| | | TCGAGTGGGAGCTGCAGAAGGAAAACAGCAAGCGCTGGAACCCGGAGATCCAGTACACT |
| | | TCCAACTATTACAAGTCTAATAATGTTGAATTTGCTGTTAATACTGAAGGTGTATATAG |
| | | TGAACCCCGCCCCATTGGCACGCGGTATTTAACGAGGAACTTA |
| TTM-020 | 29 | ATGGCTGCCGATGGTTATCTTCCAGATTGGCTCGAGGACAACCTTAGTGAAGGAATTCG |
| | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGATCAGTATCTGTATTACTTGAGTAAGACGATTAATG |
| | | GTCATGATTCTCCGCATTCTAAGGCGCTGAATCAGCAGACGTTGAAGTTTTCGGTAGCT |
| | | GGTCCTAGCAACATGGCTGTCCAGGGAAGAAACTACATACCTGGACCCAGCTACCGACA |
| | | ACAACGTGTCTCAACCACTGTGACTCAAAACAACAACAGCGAATTTGCTTGGCCTGGAG |
| | | CTTCTTCTTGGGCTCTCAATGGACGTAATAGCTTGATGAATCCTGGACCTGCTATGGCC |
| | | AGCCACAAAGAAGGAGAGGACCGTTTCTTTCCTTTGTCTGGATCTTTAATTTTTGGCAA |
| | | ACAAGGAACTGGAAGAGACAACGTGGATGCGGACAAAGTCATGATAACCAACGAAGAAG |
| | | AAATTAAAACTACTAACCCGGTAGCAACGGAGTCCTATGGACAAGTGGCCACAAACCAC |
| | | CAGAGTGCCCAAGCACAGGCGCAGACCGGCTGGGTTCAAAACCAAGGAATACTTCCGGG |
| | | TATGGTTTGGCAGGACAGAGATGTGTACCTGCAAGGACCCATTTGGGCCAAAATTCCTC |
| | | ACACGGACGGCAACTTTCACCCTTCTCCGCTGATGGGAGGGTTTGGAATGAAGCACCCG |
| | | CCTCCTCAGATCCTCATCAAAAACACACCTGTACCTGCGGATCCTCCAACGGCCTTCAA |
| | | CAAGGACAAGCTGAACTCTTTCATCACCCAGTATTCTACTGGCCAAGTCAGCGTGGAGA |
| | | TCGAGTGGGAGCTGCAGAAGGAAAACAGCAAGCGCTGGAACCCGGAGATCCAGTACACT |
| | | TCCAACTATTACAAGTCTAATAATGTTGAATTTGCTGTTAATACTGAAGGTGTATATAG |
| | | TGAACCCCGCCCCATTGGCACGCGGTATTTAACGAGGAACTTA |
| TTM-021 | 30 | ATGGCTGCCGATGGTTATCTTCCAGATTGGCTCGAGGACAACCTTAGTGAAGGAATTCG |
| | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGATCAGTATCTGTATTACTTGAGTAAGACGATTAATG |
| | | GTCATGATTCTCCGCATTCTAAGGCGCAGAATCAGCAGTCTTTGAAGTTTTCGGTAGCT |
| | | GGTCCTAGCAACATGGCTGTCCAGGGAAGAAACTACATACCTGGACCCAGCTACCGACA |
| | | ACAACGTGTCTCAACCACTGTGACTCAAAACAACAACAGCGAATTTGCTTGGCCTGGAG |
| | | CTTCTTCTTGGGCTCTCAATGGACGTAATAGCTTGATGAATCCTGGACCTGCTATGGCC |
| | | AGCCACAAAGAAGGAGAGGACCGTTTCTTTCCTTTGTCTGGATCTTTAATTTTTGGCAA |
| | | ACAAGGAACTGGAAGAGACAACGTGGATGCGGACAAAGTCATGATAACCAACGAAGAAG |
| | | AAATTAAAACTACTAACCCGGTAGCAACGGAGTCCTATGGACAAGTGGCCACAAACCAC |
| | | CAGAGTGCCCAAGCACAGGCGCAGACCGGCTGGGTTCAAAACCAAGGAATACTTCCGGG |
| | | TATGGTTTGGCAGGACAGAGATGTGTACCTGCAAGGACCCATTTGGGCCAAAATTCCTC |
| | | ACACGGACGGCAACTTTCACCCTTCTCCGCTGATGGGAGGGTTTGGAATGAAGCACCCG |
| | | CCTCCTCAGATCCTCATCAAAAACACACCTGTACCTGCGGATCCTCCAACGGCCTTCAA |
| | | CAAGGACAAGCTGAACTCTTTCATCACCCAGTATTCTACTGGCCAAGTCAGCGTGGAGA |
| | | TCGAGTGGGAGCTGCAGAAGGAAAACAGCAAGCGCTGGAACCCGGAGATCCAGTACACT |
| | | TCCAACTATTACAAGTCTAATAATGTTGAATTTGCTGTTAATACTGAAGGTGTATATAG |
| | | TGAACCCCGCCCCATTGGCACGCGGTATTTAACGAGGAACTTA |
| TTM-022 | 31 | ATGGCTGCCGATGGTTATCTTCCAGATTGGCTCGAGGACAACCTTAGTGAAGGAATTCG |
| | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGATCAGTATCTGTATTACTTGAGTAAGACGATTAATG |
| | | GTCATGATTCTCCGCATTCTAAGGCGCAGAATCAGCAGATTTTGAAGTTTTCGGTAGCT |
| | | GGTCCTAGCAACATGGCTGTCCAGGGAAGAAACTACATACCTGGACCCAGCTACCGACA |
| | | ACAACGTGTCTCAACCACTGTGACTCAAAACAACAACAGCGAATTTGCTTGGCCTGGAG |
| | | CTTCTTCTTGGGCTCTCAATGGACGTAATAGCTTGATGAATCCTGGACCTGCTATGGCC |
| | | AGCCACAAAGAAGGAGAGGACCGTTTCTTTCCTTTGTCTGGATCTTTAATTTTTGGCAA |
| | | ACAAGGAACTGGAAGAGACAACGTGGATGCGGACAAAGTCATGATAACCAACGAAGAAG |
| | | AAATTAAAACTACTAACCCGGTAGCAACGGAGTCCTATGGACAAGTGGCCACAAACCAC |
| | | CAGAGTGCCCAAGCACAGGCGCAGACCGGCTGGGTTCAAAACCAAGGAATACTTCCGGG |
| | | TATGGTTTGGCAGGACAGAGATGTGTACCTGCAAGGACCCATTTGGGCCAAAATTCCTC |
| | | ACACGGACGGCAACTTTCACCCTTCTCCGCTGATGGGAGGGTTTGGAATGAAGCACCCG |
| | | CCTCCTCAGATCCTCATCAAAAACACACCTGTACCTGCGGATCCTCCAACGGCCTTCAA |
| | | CAAGGACAAGCTGAACTCTTTCATCACCCAGTATTCTACTGGCCAAGTCAGCGTGGAGA |
| | | TCGAGTGGGAGCTGCAGAAGGAAAACAGCAAGCGCTGGAACCCGGAGATCCAGTACACT |
| | | TCCAACTATTACAAGTCTAATAATGTTGAATTTGCTGTTAATACTGAAGGTGTATATAG |
| | | TGAACCCCGCCCCATTGGCACGCGGTATTTAACGAGGAACTTA |
| TTM-023 | 32 | ATGGCTGCCGATGGTTATCTTCCAGATTGGCTCGAGGACAACCTTAGTGAAGGAATTCG |
| | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGATCAGTATCTGTATTACTTGAGTAAGACGATTAATG |
| | | GTTCTGGTTCTCCGCATTTTACGCGTCAGAATCAGCAGACGITGAAGTTTTCGGTAGCT |
| | | GGTCCTAGCAACATGGCTGTCCAGGGAAGAAACTACATACCTGGACCCAGCTACCGACA |
| | | ACAACGTGTCTCAACCACTGTGACTCAAAACAACAACAGCGAATTTGCTTGGCCTGGAG |
| | | CTTCTTCTTGGGCTCTCAATGGACGTAATAGCTTGATGAATCCTGGACCTGCTATGGCC |
| | | AGCCACAAAGAAGGAGAGGACCGTTTCTTTCCTTTGTCTGGATCTTTAATTTTTGGCAA |
| | | ACAAGGAACTGGAAGAGACAACGTGGATGCGGACAAAGTCATGATAACCAACGAAGAAG |
| | | AAATTAAAACTACTAACCCGGTAGCAACGGAGTCCTATGGACAAGTGGCCACAAACCAC |
| | | CAGAGTGCCCAAGCACAGGCGCAGACCGGCTGGGTTCAAAACCAAGGAATACTTCCGGG |
| | | TATGGTTTGGCAGGACAGAGATGTGTACCTGCAAGGACCCATTTGGGCCAAAATTCCTC |
| | | ACACGGACGGCAACTTTCACCCTTCTCCGCTGATGGGAGGGTTTGGAATGAAGCACCCG |
| | | CCTCCTCAGATCCTCATCAAAAACACACCTGTACCTGCGGATCCTCCAACGGCCTTCAA |
| | | CAAGGACAAGCTGAACTCTTTCATCACCCAGTATTCTACTGGCCAAGTCAGCGTGGAGA |
| | | TCGAGTGGGAGCTGCAGAAGGAAAACAGCAAGCGCTGGAACCCGGAGATCCAGTACACT |
| | | TCCAACTATTACAAGTCTAATAATGTTGAATTTGCTGTTAATACTGAAGGTGTATATAG |
| | | TGAACCCCGCCCCATTGGCACGCGGTATTTAACGAGGAACTTA |
| TTM-024 | 33 | ATGGCTGCCGATGGTTATCTTCCAGATTGGCTCGAGGACAACCTTAGTGAAGGAATTCG |
| | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGATCAGTATCTGTATTACTTGAGTAAGACGTCTAATG |
| | | GTCATGATTCTCCGCATTCTAAGGCGCAGAATCAGCAGACGTTGAAGTTTTCGGTAGCT |
| | | GGTCCTAGCAACATGGCTGTCCAGGGAAGAAACTACATACCTGGACCCAGCTACCGACA |
| | | ACAACGTGTCTCAACCACTGTGACTCAAAACAACAACAGCGAATTTGCTTGGCCTGGAG |
| | | CTTCTTCTTGGGCTCTCAATGGACGTAATAGCTTGATGAATCCTGGACCTGCTATGGCC |
| | | AGCCACAAAGAAGGAGAGGACCGTTTCTTTCCTTTGTCTGGATCTTTAATTTTTGGCAA |
| | | ACAAGGAACTGGAAGAGACAACGTGGATGCGGACAAAGTCATGATAACCAACGAAGAAG |
| | | AAATTAAAACTACTAACCCGGTAGCAACGGAGTCCTATGGACAAGTGGCCACAAACCAC |
| | | CAGAGTGCCCAAGCACAGGCGCAGACCGGCTGGGTTCAAAACCAAGGAATACTTCCGGG |
| | | TATGGTTTGGCAGGACAGAGATGTGTACCTGCAAGGACCCATTTGGGCCAAAATTCCTC |
| | | ACACGGACGGCAACTTTCACCCTTCTCCGCTGATGGGAGGGTTTGGAATGAAGCACCCG |
| | | CCTCCTCAGATCCTCATCAAAAACACACCTGTACCTGCGGATCCTCCAACGGCCTTCAA |
| | | CAAGGACAAGCTGAACTCTTTCATCACCCAGTATTCTACTGGCCAAGTCAGCGTGGAGA |
| | | TCGAGTGGGAGCTGCAGAAGGAAAACAGCAAGCGCTGGAACCCGGAGATCCAGTACACT |
| | | TCCAACTATTACAAGTCTAATAATGTTGAATTTGCTGTTAATACTGAAGGTGTATATAG |
| | | TGAACCCCGCCCCATTGGCACGCGGTATTTAACGAGGAACTTA |
| TTM-025 | 34 | ATGGCTGCCGATGGTTATCTTCCAGATTGGCTCGAGGACAACCTTAGTGAAGGAATTCG |
| | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGATCAGTATCTGTATTACTTGAGTAAGACGATTAATG |
| | | GTTCTGGTTCTCCGCATTCTCTGCCGTGGAATCAGCAGACGTTGAAGTTTTCGGTAGCT |
| | | GGTCCTAGCAACATGGCTGTCCAGGGAAGAAACTACATACCTGGACCCAGCTACCGACA |
| | | ACAACGTGTCTCAACCACTGTGACTCAAAACAACAACAGCGAATTTGCTTGGCCTGGAG |
| | | CTTCTTCTTGGGCTCTCAATGGACGTAATAGCTTGATGAATCCTGGACCTGCTATGGCC |
| | | AGCCACAAAGAAGGAGAGGACCGTTTCTTTCCTTTGTCTGGATCTTTAATTTTTGGCAA |
| | | ACAAGGAACTGGAAGAGACAACGTGGATGCGGACAAAGTCATGATAACCAACGAAGAAG |
| | | AAATTAAAACTACTAACCCGGTAGCAACGGAGTCCTATGGACAAGTGGCCACAAACCAC |
| | | CAGAGTGCCCAAGCACAGGCGCAGACCGGCTGGGTTCAAAACCAAGGAATACTTCCGGG |
| | | TATGGTTTGGCAGGACAGAGATGTGTACCTGCAAGGACCCATTTGGGCCAAAATTCCTC |
| | | ACACGGACGGCAACTTTCACCCTTCTCCGCTGATGGGAGGGTTTGGAATGAAGCACCCG |
| | | CCTCCTCAGATCCTCATCAAAAACACACCTGTACCTGCGGATCCTCCAACGGCCTTCAA |
| | | CAAGGACAAGCTGAACTCTTTCATCACCCAGTATTCTACTGGCCAAGTCAGCGTGGAGA |
| | | TCGAGTGGGAGCTGCAGAAGGAAAACAGCAAGCGCTGGAACCCGGAGATCCAGTACACT |
| | | TCCAACTATTACAAGTCTAATAATGTTGAATTTGCTGTTAATACTGAAGGTGTATATAG |
| | | TGAACCCCGCCCCATTGGCACGCGGTATTTAACGAGGAACTTA |
| TTM-026 | 35 | ATGGCTGCCGATGGTTATCTTCCAGATTGGCTCGAGGACAACCTTAGTGAAGGAATTCG |
| | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGATCAGTATCTGTATTACTTGAGTAAGACGATTAATG |
| | | GTCATGATTCTCCGCATTCTAAGGCGCAGAATCATCAGACGTTGAAGTTTTCGGTAGCT |
| | | GGTCCTAGCAACATGGCTGTCCAGGGAAGAAACTACATACCTGGACCCAGCTACCGACA |
| | | ACAACGTGTCTCAACCACTGTGACTCAAAACAACAACAGCGAATTTGCTTGGCCTGGAG |
| | | CTTCTTCTTGGGCTCTCAATGGACGTAATAGCTTGATGAATCCTGGACCTGCTATGGCC |
| | | AGCCACAAAGAAGGAGAGGACCGTTTCTTTCCTTTGTCTGGATCTTTAATTTTTGGCAA |
| | | ACAAGGAACTGGAAGAGACAACGTGGATGCGGACAAAGTCATGATAACCAACGAAGAAG |
| | | AAATTAAAACTACTAACCCGGTAGCAACGGAGTCCTATGGACAAGTGGCCACAAACCAC |
| | | CAGAGTGCCCAAGCACAGGCGCAGACCGGCTGGGTTCAAAACCAAGGAATACTTCCGGG |
| | | TATGGTTTGGCAGGACAGAGATGTGTACCTGCAAGGACCCATTTGGGCCAAAATTCCTC |
| | | ACACGGACGGCAACTTTCACCCTTCTCCGCTGATGGGAGGGTTTGGAATGAAGCACCCG |
| | | CCTCCTCAGATCCTCATCAAAAACACACCTGTACCTGCGGATCCTCCAACGGCCTTCAA |
| | | CAAGGACAAGCTGAACTCTTTCATCACCCAGTATTCTACTGGCCAAGTCAGCGTGGAGA |
| | | TCGAGTGGGAGCTGCAGAAGGAAAACAGCAAGCGCTGGAACCCGGAGATCCAGTACACT |
| | | TCCAACTATTACAAGTCTAATAATGTTGAATTTGCTGTTAATACTGAAGGTGTATATAG |
| | | TGAACCCCGCCCCATTGGCACGCGGTATTTAACGAGGAACTTA |
| TTM-027 | 9 | ATGGCTGCCGATGGTTATCTTCCAGATTGGCTCGAGGACAACCTTAGTGAAGGAATTCG |
| | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGACCAATACTTGTACTATCTCTCTAAGACTGAGAATG |
| | | TGAGCGGGAGCCCTCATAGCAAGGCTCAGAATCAGCAGACTCTAAAATTCAGTGTGGCC |
| | | GGACCCAGCAACATGGCTGTCCAGGGAAGAAACTACATACCTGGACCCAGCTACCGACA |
| | | ACAACGTGTCTCAACCACTGTGACTCAAAACAACAACAGCGAATTTGCTTGGCCTGGAG |
| | | CTTCTTCTTGGGCTCTCAATGGACGTAATAGCTTGATGAATCCTGGACCTGCTATGGCC |
| | | AGCCACAAAGAAGGAGAGGACCGTTTCTTTCCTTTGTCTGGATCTTTAATTTTTGGCAA |
| | | ACAAGGAACTGGAAGAGACAACGTGGATGCGGACAAAGTCATGATAACCAACGAAGAAG |
| | | AAATTAAAACTACTAACCCGGTAGCAACGGAGTCCTATGGACAAGTGGCCACAAACCAC |
| | | CAGAGTGCCCAAGCACAGGCGCAGACCGGCTGGGTTCAAAACCAAGGAATACTTCCGGG |
| | | TATGGTTTGGCAGGACAGAGATGTGTACCTGCAAGGACCCATTTGGGCCAAAATTCCTC |
| | | ACACGGACGGCAACTTTCACCCTTCTCCGCTGATGGGAGGGTTTGGAATGAAGCACCCG |
| | | CCTCCTCAGATCCTCATCAAAAACACACCTGTACCTGCGGATCCTCCAACGGCCTTCAA |
| | | CAAGGACAAGCTGAACTCTTTCATCACCCAGTATTCTACTGGCCAAGTCAGCGTGGAGA |
| | | TCGAGTGGGAGCTGCAGAAGGAAAACAGCAAGCGCTGGAACCCGGAGATCCAGTACACT |
| | | TCCAACTATTACAAGTCTAATAATGTTGAATTTGCTGTTAATACTGAAGGTGTATATAG |
| | | TGAACCCCGCCCCATTGGCACCAGATACCTGACTCGTAATCTG |

In some embodiments, the polynucleotide encoding an AAV capsid variant, described herein comprises the nucleotide sequence of SEQ ID NO: 983 or 984, or a nucleotide sequence with at least 70% (e.g., at least about 80, 85, 90, 95, 96, 97, 98, or 99%) sequence identity thereto.

In some embodiments, the polynucleotide encoding an AAV capsid variant, described herein comprises the nucleotide sequence of any one of SEQ ID NOs: 9, 72, 73, 74, 75, 76, 77, 78, 79, 80, 81, or 22-35, or a nucleotide sequence with at least 70% (e.g., at least about 80, 85, 90, 95, 96, 97, 98, or 99%) sequence identity thereto.

In some embodiments, the polynucleotide encoding an AAV capsid variant described herein comprises the nucleotide sequence of SEQ ID NO: 983, or a nucleotide sequence with at least 70% (e.g., at least about 80, 85, 90, 95, 96, 97, 98, or 99%) sequence identity thereto. In some embodiments, the nucleotide sequence encoding an AAV capsid variant described herein, comprises a nucleotide sequence comprising at least one, two or three modifications, e.g., substitutions (e.g., conservative substitutions), insertions, or deletions, but not more than 30, 20 or 10 modifications, e.g., substitutions (e.g., conservative substitutions), insertions, or deletions, relative to the nucleotide sequence of SEQ ID NO: 983. In some embodiments, the nucleotide sequence encoding an AAV capsid variant described herein, comprises a nucleotide sequence comprising at least one, two or three, but not more than 30, 20 or 10 different nucleotides relative to the amino acid sequence of SEQ ID NO: 983. In some embodiments, the nucleic acid sequence encoding an AAV capsid variant described herein is codon optimized.

In some embodiments, the polynucleotide encoding an AAV capsid variant described herein comprises the nucleotide sequence of SEQ ID NO: 984, or a nucleotide sequence with at least 70% (e.g., at least about 80, 85, 90, 95, 96, 97, 98, or 99%) sequence identity thereto. In some embodiments, the nucleotide sequence encoding an AAV capsid variant described herein, comprises a nucleotide sequence comprising at least one, two or three modifications, e.g., substitutions (e.g., conservative substitutions), insertions, or deletions, but not more than 30, 20 or 10 modifications, e.g., substitutions (e.g., conservative substitutions), insertions, or deletions, relative to the nucleotide sequence of SEQ ID NO: 984. In some embodiments, the nucleotide sequence encoding an AAV capsid variant described herein, comprises a nucleotide sequence comprising at least one, two or three, but not more than 30, 20 or 10 different nucleotides, relative to the amino acid sequence of SEQ ID NO: 984. In some embodiments, the nucleic acid sequence encoding an AAV capsid variant described herein is codon optimized.

In some embodiments, an AAV capsid variant described herein comprises the amino acid sequence of SEQ ID NO: 69, 70, 71, 4, 5, 6, 7, 8, 36-59, 981, or 982, or an amino acid sequence with at least 70% (e.g., at least about 80, 85, 90, 95, 96, 97, 98, or 99%) sequence identity thereto. In some embodiments, an AAV capsid variant described herein comprises an amino acid sequence comprising at least one, two, or three modifications, e.g., substitutions (e.g., conservative substitutions), insertions, or deletions, but not more than 30, 20 or 10 modifications, e.g., substitutions (e.g., conservative substitutions), insertions, or deletions, relative to the amino acid sequence of SEQ ID NO: 69, 70, 71, 4, 5, 6, 7, 8, 36-59, 981, or 982. In some embodiments, the AAV capsid variant, comprises an amino acid sequence comprising at least one, two or three, but not more than 30, 20 or 10 different amino acids, relative to the amino acid sequence of SEQ ID NO: 69, 70, 71, 4, 5, 6, 7, 8, 36-59, 981, or 982.

In some embodiments, an AAV capsid variant described herein comprises the amino acid sequence of SEQ ID NO: 36, or an amino acid sequence with at least 70% (e.g., at least about 80, 85, 90, 95, 96, 97, 98, or 99%) sequence identity thereto. In some embodiments, an AAV capsid variant described herein comprises an amino acid sequence comprising at least one, two, or three modifications, e.g., substitutions (e.g., conservative substitutions), insertions, or deletions, but not more than 30, 20 or 10 modifications, e.g., substitutions (e.g., conservative substitutions), insertions, or deletions, relative to the amino acid sequence of SEQ ID NO: 36. In some embodiments, the AAV capsid variant, comprises an amino acid sequence comprising at least one, two or three, but not more than 30, 20 or 10 different amino acids, relative to the amino acid sequence of SEQ ID NO: 36.

In some embodiments, an AAV capsid variant described herein comprises the amino acid sequence of SEQ ID NO: 39, or an amino acid sequence with at least 70% (e.g., at least about 80, 85, 90, 95, 96, 97, 98, or 99%) sequence identity thereto. In some embodiments, an AAV capsid variant described herein comprises an amino acid sequence comprising at least one, two, or three modifications, e.g., substitutions (e.g., conservative substitutions), insertions, or deletions, but not more than 30, 20 or 10 modifications, e.g., substitutions (e.g., conservative substitutions), insertions, or deletions, relative to the amino acid sequence of SEQ ID NO: 39. In some embodiments, the AAV capsid variant, comprises an amino acid sequence comprising at least one, two or three, but not more than 30, 20 or 10 different amino acids, relative to the amino acid sequence of SEQ ID NO: 39.

In some embodiments, an AAV capsid variant described herein comprises the amino acid sequence of SEQ ID NO: 51, or an amino acid sequence with at least 70% (e.g., at least about 80, 85, 90, 95, 96, 97, 98, or 99%) sequence identity thereto. In some embodiments, an AAV capsid variant described herein comprises an amino acid sequence comprising at least one, two, or three modifications, e.g., substitutions (e.g., conservative substitutions), insertions, or deletions, but not more than 30, 20 or 10 modifications, e.g., substitutions (e.g., conservative substitutions), insertions, or deletions, relative to the amino acid sequence of SEQ ID NO: 51. In some embodiments, the AAV capsid variant, comprises an amino acid sequence comprising at least one, two or three, but not more than 30, 20 or 10 different amino acids, relative to the amino acid sequence of SEQ ID NO: 51.

In some embodiments, an AAV capsid variant described herein comprises the amino acid sequence of SEQ ID NO: 52, or an amino acid sequence with at least 70% (e.g., at least about 80, 85, 90, 95, 96, 97, 98, or 99%) sequence identity thereto. In some embodiments, an AAV capsid variant described herein comprises an amino acid sequence comprising at least one, two, or three modifications, e.g., substitutions (e.g., conservative substitutions), insertions, or deletions, but not more than 30, 20 or 10 modifications, e.g., substitutions (e.g., conservative substitutions), insertions, or deletions, relative to the amino acid sequence of SEQ ID NO: 52. In some embodiments, the AAV capsid variant, comprises an amino acid sequence comprising at least one, two or three, but not more than 30, 20 or 10 different amino acids, relative to the amino acid sequence of SEQ ID NO: 52.

In some embodiments, an AAV capsid variant described herein comprises the amino acid sequence of SEQ ID NO: 69, or an amino acid sequence with at least 70% (e.g., at least about 80, 85, 90, 95, 96, 97, 98, or 99%) sequence identity thereto. In some embodiments, an AAV capsid variant described herein comprises an amino acid sequence comprising at least one, two, or three modifications, e.g., substitutions (e.g., conservative substitutions), insertions, or deletions, but not more than 30, 20 or 10 modifications, e.g., substitutions (e.g., conservative substitutions), insertions, or deletions, relative to the amino acid sequence of SEQ ID NO: 69. In some embodiments, the AAV capsid variant, comprises an amino acid sequence comprising at least one, two or three, but not more than 30, 20 or 10 different amino acids, relative to the amino acid sequence of SEQ ID NO: 69. In some embodiments, the AAV capsid variant comprises an amino acid sequence at least 90% identical to SEQ ID NO: 69. In some embodiments, the AAV capsid variant comprises an amino acid sequence at least 95% identical to SEQ ID NO: 69. In some embodiments, the AAV capsid variant comprises an amino acid sequence at least 97% identical to SEQ ID NO: 69. In some embodiments, the AAV capsid variant comprises an amino acid sequence at least 98% identical to SEQ ID NO: 69. In some embodiments, the AAV capsid variant comprises an amino acid sequence at least 99% identical to SEQ ID NO: 69. In some embodiments, the AAV capsid variant comprises the amino acid sequence of SEQ ID NO: 69.

In some embodiments, an AAV capsid variant described herein, comprises the amino acid sequence of SEQ ID NO: 981, or an amino acid sequence with at least 70% (e.g., at least about 80, 85, 90, 95, 96, 97, 98, or 99%) sequence identity thereto. In some embodiments, an AAV capsid variant described herein comprises an amino acid sequence comprising at least one, two, or three modifications, e.g., substitutions (e.g., conservative substitutions), insertions, or deletions, but not more than 30, 20 or 10 modifications, e.g., substitutions (e.g., conservative substitutions), insertions, or deletions, relative to the amino acid sequence of SEQ ID NO: 981. In some embodiments, an AAV capsid variant described herein comprises an amino acid sequence comprising at least one, two or three, but not more than 30, 20 or 10 different amino acids, relative to the amino acid sequence of SEQ ID NO: 981.

In some embodiments, an AAV capsid variant described herein comprises the amino acid sequence of SEQ ID NO: 982, or an amino acid sequence with at least 70% (e.g., at least about 80, 85, 90, 95, 96, 97, 98, or 99%) sequence identity thereto. In some embodiments, an AAV capsid variant described herein comprises an amino acid sequence comprising at least one, two, or three modifications, e.g., substitutions (e.g., conservative substitutions), insertions, or deletions, but not more than 30, 20 or 10 modifications, e.g., substitutions (e.g., conservative substitutions), insertions, or deletions, relative to the amino acid sequence of SEQ ID NO: 982. In some embodiments, the AAV capsid variant, comprises an amino acid sequence comprising at least one, two or three, but not more than 30, 20 or 10 different amino acids, relative to the amino acid sequence of SEQ ID NO: 982.

In some embodiments, an AAV capsid variant described herein comprises an amino acid sequence encoded by the nucleotide sequence of SEQ ID NO: 983 or 984, or a nucleotide sequence with at least 70% (e.g., at least about 80, 85, 90, 95, 96, 97, 98, or 99%) sequence identity thereto. In some embodiments, an AAV capsid variant described herein comprises an amino acid sequence encoded by a nucleotide sequence comprising at least one, two or three, but not more than 30, 20 or 10 different nucleotides, relative to the amino acid sequence of SEQ ID NO: 983 or 984. In some embodiments, an AAV capsid variant described herein comprises an amino acid sequence encoded by a nucleotide sequence comprising at least one, two or three modifications, e.g., substitutions, insertions, or deletions, but not more than 30, 20 or 10 modifications, e.g., substitutions, insertions, or deletions, relative to the nucleotide sequence of SEQ ID NO: 983 or 984.

In some embodiments, an AAV capsid variant described herein comprises an amino acid sequence encoded by the nucleotide sequence of any one of SEQ ID NOs: 9, 72, 73, 74, 75, 76, 77, 78, 79, 80, 81, or 22-35, or a nucleotide sequence with at least 70% (e.g., at least about 80, 85, 90, 95, 96, 97, 98, or 99%) sequence identity thereto. In some embodiments, an AAV capsid variant described herein comprises an amino acid sequence encoded by a nucleotide sequence comprising at least one, two or three, but not more than 30, 20 or 10 different nucleotides, relative to the amino acid sequence of any one of SEQ ID NOs: 9, 72, 73, 74, 75, 76, 77, 78, 79, 80, 81, or 22-35. In some embodiments, an AAV capsid variant described herein comprises an amino acid sequence encoded by a nucleotide sequence comprising at least one, two or three modifications, e.g., substitutions, insertions, or deletions, but not more than 30, 20 or 10 modifications, e.g., substitutions, insertions, or deletions, relative to the nucleotide sequence of any one of SEQ ID NOs: 9, 72, 73, 74, 75, 76, 77, 78, 79, 80, 81, or 22-35.

In some embodiments, an AAV capsid variant described herein comprises a VP1, VP2, VP3 protein, or a combination thereof. In some embodiments, an AAV capsid variant comprises the amino acid sequence corresponding to positions 138-742, e.g., a VP2, of SEQ ID NO: 981 or 982, or a sequence with at least 70% (e.g., at least about 80, 85, 90, 95, 96, 97, 98, or 99%) sequence identity thereto. In some embodiments, the AAV capsid protein comprises the amino acid sequence corresponding to positions 203-742, e.g., a VP3, of SEQ ID NO: 981 or 982, or a sequence with at least 70% (e.g., at least about 80, 85, 90, 95, 96, 97, 98, or 99%) sequence identity thereto. In some embodiments, the AAV capsid variant comprises the amino acid sequence corresponding to positions 1-742, e.g., a VP1, of SEQ ID NO: 981 or 982, or an amino acid sequence with at least 70% (e.g., at least about 80, 85, 90, 95, 96, 97, 98, or 99%) sequence identity thereto.

In some embodiments, an AAV capsid variant described herein comprises a VP1, VP2, VP3 protein, or a combination thereof. In some embodiments, an AAV capsid variant comprises the amino acid sequence corresponding to positions 138-742, e.g., a VP2, of any one of SEQ ID NOs: 36-59, 69, 70, 71, 4, 5, 6, 7, or 8, or a sequence with at least 70% (e.g., at least about 80, 85, 90, 95, 96, 97, 98, or 99%) sequence identity thereto. In some embodiments, the AAV capsid protein comprises the amino acid sequence corresponding to positions 203-742, e.g., a VP3, of any one of SEQ ID NOs: 36-59, 69, 70, 71, 4, 5, 6, 7, or 8, or a sequence with at least 70% (e.g., at least about 80, 85, 90, 95, 96, 97, 98, or 99%) sequence identity thereto. In some embodiments, the AAV capsid variant comprises the amino acid sequence corresponding to positions 1-742, e.g., a VP1, of any one of SEQ ID NOs: 36-59, 69, 70, 71, 4, 5, 6, 7, or 8, or an amino acid sequence with at least 70% (e.g., at least about 80, 85, 90, 95, 96, 97, 98, or 99%) sequence identity thereto.

In some embodiments, an AAV capsid variant described herein comprises a VP1, VP2, VP3 protein, or a combination thereof. In some embodiments, an AAV capsid variant comprises the amino acid sequence corresponding to positions 138-742, e.g., a VP2, of SEQ ID NO: 36, or a sequence with at least 70% (e.g., at least about 80, 85, 90, 95, 96, 97, 98, or 99%) sequence identity thereto. In some embodiments, the AAV capsid protein comprises the amino acid sequence corresponding to positions 203-742, e.g., a VP3, of SEQ ID NO: 36, or a sequence with at least 70% (e.g., at least about 80, 85, 90, 95, 96, 97, 98, or 99%) sequence identity thereto. In some embodiments, the AAV capsid variant comprises the amino acid sequence corresponding to positions 1-742, e.g., a VP1, of SEQ ID NO: 36, or an amino acid sequence with at least 70% (e.g., at least about 80, 85, 90, 95, 96, 97, 98, or 99%) sequence identity thereto.

In some embodiments, an AAV capsid variant described herein comprises a VP1, VP2, VP3 protein, or a combination thereof. In some embodiments, an AAV capsid variant comprises the amino acid sequence corresponding to positions 138-742, e.g., a VP2, of SEQ ID NO: 69, or a sequence with at least 70% (e.g., at least about 80, 85, 90, 95, 96, 97, 98, or 99%) sequence identity thereto. In some embodiments, the AAV capsid protein comprises the amino acid sequence corresponding to positions 203-742, e.g., a VP3, of SEQ ID NO: 69, or a sequence with at least 70% (e.g., at least about 80, 85, 90, 95, 96, 97, 98, or 99%) sequence identity thereto. In some embodiments, the AAV capsid variant comprises the amino acid sequence corresponding to positions 1-742, e.g., a VP1, of SEQ ID NO: 69, or an amino acid sequence with at least 70% (e.g., at least about 80, 85, 90, 95, 96, 97, 98, or 99%) sequence identity thereto.

In some embodiments, the AAV capsid variant comprises amino acids 203-742 of SEQ ID NO: 36, or an amino acid sequence at least 70%, 75%, 80%, 85%, 90%, 92%, 95%, 96%, 97%, 98%, or 99% identical thereto. In some embodiments, the AAV capsid variant comprises amino acids 138-742 of SEQ ID NO: 36, or an amino acid sequence at least 70%, 75%, 80%, 85%, 90%, 92%, 95%, 96%, 97%, 98%, or 99% identical thereto. In some embodiments, the AAV capsid variant comprises the amino acid sequence of SEQ ID NO: 36, or an amino acid sequence at least 70%, 75%, 80%, 85%, 90%, 92%, 95%, 96%, 97%, 98%, or 99% identical thereto.

In some embodiments, the AAV capsid variant comprises amino acids 203-742 of SEQ ID NO: 69, or an amino acid sequence at least 70%, 75%, 80%, 85%, 90%, 92%, 95%, 96%, 97%, 98%, or 99% identical thereto. In some embodiments, the AAV capsid variant comprises amino acids 138-742 of SEQ ID NO: 69, or an amino acid sequence at least 70%, 75%, 80%, 85%, 90%, 92%, 95%, 96%, 97%, 98%, or 99% identical thereto. In some embodiments, the AAV capsid variant comprises the amino acid sequence of SEQ ID NO: 69, or an amino acid sequence at least 70%, 75%, 80%, 85%, 90%, 92%, 95%, 96%, 97%, 98%, or 99% identical thereto.

In some embodiments, an AAV capsid variant, described herein has an increased tropism for a CNS cell or tissue, e.g., a brain cell, brain tissue, spinal cord cell, or spinal cord tissue, relative to the tropism of a reference sequence comprising the amino acid sequence of SEQ ID NO: 138.

In some embodiments, an AAV capsid variant described herein transduces a brain region, e.g., a midbrain region (e.g., the hippocampus, or thalamus) or the brain stem. In some embodiments, the level of transduction is at least 5, 10, 15, 20, 25, 30, 35, 40, 45, 50, 55, 60, or 65-fold greater as compared to a reference sequence of SEQ ID NO: 138. In some embodiments, the level of transduction is at least 30, 35, 40, 45, 50, 55, 60, or 65-fold greater as compared to a reference sequence of SEQ ID NO: 138.

In some embodiments, an AAV capsid variant described herein is enriched at least about 3, 4, 5, 6, 7, 8, 9, or 10-fold in the brain compared to a reference sequence of SEQ ID NO: 138. In some embodiments, an AAV capsid variant described herein is enriched at least about 20, 25, 30, 35, 40, 45, 50, 55, 60, 65, 70, 75, 80, or 85-fold in the brain compared to a reference sequence of SEQ ID NO: 138.

In some embodiments, an AAV capsid variant described herein is enriched in the brain of at least two to three species, e.g., a non-human primate and rodent (e.g., mouse) species, compared to a reference sequence of SEQ ID NO: 138. In some embodiments, an AAV capsid variant described herein is enriched at least about 10, 15, 20, 25, 30, 35, 40, 45, 50, 55, 60, 65, 70, 75, 80, 85, 90, 95, or 100-fold in the brain of at least two to three species, e.g., a non-human primate and rodent (e.g., mouse) species, compared to a reference sequence of SEQ ID NO: 138. In some embodiments, the at least two to three species are Macaca fascicularis, Chlorocebus sabaeus, Callithrix jacchus, and/or mouse (e.g., BALB/c mice, C57BI/6 mice, and/or CD-1 outbred mice).

In some embodiments, an AAV capsid variant described herein is enriched at least about 2, 2.5, 3, 3.5, 4, 4.5, 5, 5.5, 6, 6.5, 7, 7.5, or 8-fold, in the brain compared to a reference sequence of SEQ ID NO: 981. In some embodiments, an AAV capsid variant described herein is enriched about 2, 2.5, 3, 3.5, 4, 4.5, 5, or 5.5-fold, in the brain compared to a reference sequence of SEQ ID NO: 982.

In some embodiments, an AAV capsid variant described herein delivers an increased level of viral genomes to a brain region. In some embodiments, the level of viral genomes is increased by at least 20, 25, 30, 35, 40, 45, or 50-fold, as compared to a reference sequence of SEQ ID NO: 138. In some embodiments, the brain region comprises a midbrain region (e.g., the hippocampus or thalamus) and/or the brainstem.

In some embodiments, an AAV capsid variant described herein delivers an increased level of a payload to a brain region. In some embodiments, the level of the payload is increased by at least 20, 25, 30, 35, 40, 45, 50, 55, 60, 65, or 70-fold, as compared to a reference sequence of SEQ ID NO: 138. In some embodiments, the brain region comprises a midbrain region (e.g., the hippocampus or thalamus) and/or the brainstem.

In some embodiments, an AAV capsid variant described herein is enriched at least about 5, 10, 15, 20, 25, 30, or 35-fold, in the spinal cord compared to a reference sequence of SEQ ID NO: 138.

In some embodiments, an AAV capsid variant described herein shows preferential transduction in a brain region relative to the transduction in the dorsal root ganglia (DRG). In some embodiments, the AAV capsid variant shows preferential transduction in a brain region relative to the transduction in the liver. In some embodiments, the AAV capsid variant shows preferential transduction in a brain region relative to the transduction in the liver and the DRG. In some embodiments, the AAV capsid variant shows preferential transduction in a brain region relative to the transduction in the heart. In some embodiments, the AAV capsid variant shows preferential transduction in a brain region relative to the transduction in the heart, and DRG. In some embodiments, the AAV capsid variant shows preferential transduction in a brain region relative to the transduction in the heart, DRG, and liver.

In some embodiments, an AAV capsid variant described herein is capable of transducing non-neuronal cells, e.g., glial cells (e.g., oligodendrocytes or astrocytes). In some embodiments, the AAV capsid variant described herein is capable of transducing neuronal cells and non-neuronal cells, e.g., glial cells (e.g., oligodendrocytes or astrocytes). In some embodiments, the non-neuronal cells are glial cells, oligodendrocytes (e.g., Olig2 positive oligodendrocytes), or astrocytes (e.g., Olig2 positive astrocytes). In some embodiments, the AAV capsid variant is capable of transducing Olig2 positive cells, e.g., Olig2 positive astrocytes or Olig2 positive oligodendrocytes.

In some embodiments, an AAV capsid polypeptide, e.g., an AAV capsid variant, of the present disclosure has decreased tropism for the liver. In some embodiments, an AAV capsid variant comprises a modification, e.g., substitution (e.g., conservative substitution), insertion, or deletion, that results in reduced tropism (e.g., de-targeting) and/or activity in the liver. In some embodiments, the reduced tropism in the liver is compared to an otherwise similar capsid that does not comprise the modification, e.g., a wild-type capsid polypeptide. In some embodiments, an AAV capsid variant described comprises a modification, e.g., substitution (e.g., conservative substitution), insertion, or deletion, that results in one or more of the following properties: (1) reduced tropism in the liver; (2) de-targeted expression in the liver; (3) reduced activity in the liver; and/or (4) reduced binding to galactose. In some embodiments, the reduction in any one, or all of properties (1)-(3) is compared to an otherwise similar AAV capsid variant that does not comprise the modification. Exemplary modifications are provided in WO 2018/119330; Pulicherla et al. (2011) Mol. Ther. 19 (6): 1070-1078; Adachi et al. (2014) Nature Communications 5 (3075), DOI: 10.1038/ncomms4075; and Bell et al. (2012) J. Virol. 86 (13): 7326-33; the contents of which are hereby incorporated by reference in their entirety. In some embodiments, the AAV capsid variant comprises a modification e.g., substitution (e.g., conservative substitution), insertion, or deletion, at position N470 (e.g., N470A), D271 (e.g., D271A), N272 (e.g., N272A), Y446 (e.g., Y446A), N498 (e.g., N498Y or N498I), W503 (e.g., W503R or W503A), L620 (e.g., L620F), or a combination thereof, relative to a reference sequence numbered according to SEQ ID NO: 138. In some embodiments, the AAV capsid variant comprises one, two, three, four, five or all of an amino acid other than N at position 470 (e.g., A), an amino acid other than D at position 271 (e.g., A), an amino acid other than N at position 272 (e.g., A), an amino acid other than Y at position 446 (e.g., A), and amino acid other than N at position 498 (e.g., Y or I), and amino acid other than W at position 503 (e.g., R or A), and amino acid other than L at position 620 (e.g., F), relative to a reference sequence numbered according to SEQ ID NO: 138. In some embodiments, the AAV capsid variant comprises a modification e.g., substitution (e.g., conservative substitution), insertion, or deletion, at position N470 (e.g., N470A), D271 (e.g., D271A), N272 (e.g., N272A), Y446 (e.g., Y446A), and W503 (e.g., W503R or W503A), relative to a reference sequence numbered according to SEQ ID NO: 138. In some embodiments, the AAV capsid variant comprises a modification e.g., substitution (e.g., conservative substitution), insertion, or deletion, at N498 (e.g., N498Y) and L620 (e.g., L620F).

In some embodiments, an AAV capsid variant comprised herein comprises a modification as described in Adachi et al. (2014) Nature Communications 5 (3075), DOI: 10.1038/ncomms4075, the contents of which are hereby incorporated by reference in its entirety. Exemplary modifications that alter or do not alter tissue transduction in at least the brain, liver, heart, lung, and/or kidney can be found in Supplementary Data 2 showing the AAV Barcode-Seq data obtained with AAV9-AA−VBCLib of Adachi et al. (supra), the contents of which are hereby incorporated by reference in its entirety.

In some embodiments, an, AAV capsid polypeptide, e.g., an AAV capsid variant, of the present disclosure is isolated, e.g., recombinant. In some embodiments, a polynucleotide encoding an AAV capsid polypeptide, e.g., an AAV capsid variant, of the present disclosure is isolated, e.g., recombinant.

The present disclosure refers to structural capsid proteins (including VP1, VP2 and VP3) which are encoded by capsid (Cap) genes. These capsid proteins form an outer protein structural shell (i.e. capsid) of a viral vector such as AAV. VP capsid proteins synthesized from Cap polynucleotides generally include a methionine as the first amino acid in the peptide sequence (Met1), which is associated with the start codon (AUG or ATG) in the corresponding Cap nucleotide sequence. However, it is common for a first-methionine (Met1) residue or generally any first amino acid (AA1) to be cleaved off after or during polypeptide synthesis by protein processing enzymes such as Met-aminopeptidases. This “Met−/AA−clipping” process often correlates with a corresponding acetylation of the second amino acid in the polypeptide sequence (e.g., alanine, valine, serine, threonine, etc.). Met-clipping commonly occurs with VP1 and VP3 capsid proteins but can also occur with VP2 capsid proteins.

Where the Met−/AA−clipping is incomplete, a mixture of one or more (one, two or three) VP capsid proteins comprising the viral capsid may be produced, some of which may include a Met1/AA1 amino acid (Met+/AA+) and some of which may lack a Met1/AA1 amino acid as a result of Met−/AA−clipping (Met-/AA−). For further discussion regarding Met−/AA−clipping in capsid proteins, see Jin, et al. Direct Liquid Chromatography/Mass Spectrometry Analysis for Complete Characterization of Recombinant Adeno-Associated Virus Capsid Proteins. Hum Gene Ther Methods. 2017 Oct. 28 (5): 255-267; Hwang, et al. N-Terminal Acetylation of Cellular Proteins Creates Specific Degradation Signals. Science. 2010 Feb. 19. 327 (5968): 973-977; the contents of which are each incorporated herein by reference in its entirety.

According to the present disclosure, references to capsid proteins is not limited to either clipped (Met-/AA−) or unclipped (Met+/AA+) and may, in context, refer to independent capsid proteins, viral capsids comprised of a mixture of capsid proteins, and/or polynucleotide sequences (or fragments thereof) which encode, describe, produce or result in capsid proteins of the present disclosure. A direct reference to a capsid protein or capsid polypeptide (such as VP1, VP2 or VP2) may also comprise VP capsid proteins which include a Met1/AA1 amino acid (Met+/AA+) as well as corresponding VP capsid proteins which lack the Met1/AA1 amino acid as a result of Met−/AA−clipping (Met−/AA−).

Further according to the present disclosure, a reference to a specific SEQ ID NO: (whether a protein or nucleic acid) which comprises or encodes, respectively, one or more capsid proteins which include a Met1/AA1 amino acid (Met+/AA+) should be understood to teach the VP capsid proteins which lack the Met1/AA1 amino acid as upon review of the sequence, it is readily apparent any sequence which merely lacks the first listed amino acid (whether or not Met1/AA1).

As a non-limiting example, reference to a VP1 polypeptide sequence which is 736 amino acids in length and which includes a “Met1” amino acid (Met+) encoded by the AUG/ATG start codon may also be understood to teach a VP1 polypeptide sequence which is 735 amino acids in length and which does not include the “Met1” amino acid (Met−) of the 736 amino acid Met+sequence. As a second non-limiting example, reference to a VP1 polypeptide sequence which is 736 amino acids in length and which includes an “AA1” amino acid (AA1+) encoded by any NNN initiator codon may also be understood to teach a VP1 polypeptide sequence which is 735 amino acids in length and which does not include the “AA1” amino acid (AA1-) of the 736 amino acid AA1+sequence.

References to viral capsids formed from VP capsid proteins (such as reference to specific AAV capsid serotypes), can incorporate VP capsid proteins which include a Met1/AA1 amino acid (Met+/AA1+), corresponding VP capsid proteins which lack the Met1/AA1 amino acid as a result of Met−/AA1-clipping (Met−/AA1−), and combinations thereof (Met+/AA1+ and Met−/AA1−).

As a non-limiting example, an AAV capsid serotype can include VP1 (Met+/AA1+), VP1 (Met−/AA1−), or a combination of VP1 (Met+/AA1+) and VP1 (Met−/AA1−). An AAV capsid serotype can also include VP3 (Met+/AA1+), VP3 (Met−/AA1−), or a combination of VP3 (Met+/AA1+) and VP3 (Met−/AA1−); and can also include similar optional combinations of VP2 (Met+/AA1) and VP2 (Met−/AA1−).

Also provided herein are polynucleotide sequences encoding any of the AAV capsid variants described above and AAV particles, vectors, and cells comprising the same

### Viral Genome

In some aspects, the AAV particle of the present disclosure serves as an expression vector comprising a viral genome which encodes a SOD1 targeting siRNA. In some embodiments, expression vectors are not limited to AAV and may be adenovirus, retrovirus, lentivirus, plasmid, vector, or any variant thereof.

In some embodiments, an AAV particle, e.g., an AAV particle for the vectorized delivery of a siRNA described herein (e.g., a SOD1 targeting siRNA), comprises a viral genome, e.g., an AAV viral genome (e.g., a vector genome or AAV vector genome). In some embodiments, the viral genome, e.g., the AAV viral genome, further comprises an inverted terminal repeat (ITR) region, an enhancer, a promoter, an intron region, a Kozak sequence, an exon region, a nucleic acid encoding a payload (e.g., a SOD1 targeting siRNA described herein), a nucleotide sequence encoding a miR binding site, a poly A signal region, or a combination thereof.

In some embodiments, an AAV particle as described herein comprising an AAV capsid polypeptide, e.g., AAV capsid variant, described herein, may be used for the delivery of a viral genome to a tissue (e.g., CNS, DRG, and/or muscle). In some embodiments, an AAV particle comprising an AAV capsid polypeptide, e.g., an AAV capsid variant, described herein can be used for delivery of a viral genome to a tissue or cell, e.g., CNS, DRG, or muscle cell or tissue. In some embodiments, an AAV particle of the present disclosure is a recombinant AAV particle. In some embodiments, an AAV particle of the present disclosure is an isolated AAV particle.

The viral genome may encode any payload, such as but not limited to a polypeptide (e.g., a therapeutic polypeptide), an antibody, an enzyme, an RNAi agent and/or components of a gene editing system (e.g., a polynucleotide, e.g., a SOD1 targeting siRNA). In one embodiment, the AAV particles described herein are used to deliver a payload to cells of the CNS, after intravenous delivery. In another embodiment, the AAV particles described herein are used to deliver a payload to cells of the DRG, after intravenous delivery. In some embodiments, the AAV particles described herein are used to deliver a payload to cells of a muscle, e.g., a heart muscle, after intravenous delivery.

In some embodiments, a viral genome of an AAV particle comprising an AAV capsid polypeptide, e.g., an AAV capsid variant, as described herein, comprises a nucleic acid encoding a payload (e.g., a SOD1 targeting siRNA). In some embodiments, the viral genome comprises an inverted terminal repeat (ITR) sequence. In some embodiments, the viral genome comprises two ITR sequences, e.g., one at the 5′ end of the viral genome (e.g., 5′ relative to the encoded payload) and one at the 3′ end of the viral genome (e.g., 3′ relative to the encoded payload). In some embodiments, a viral genome of the AAV particles described herein (e.g., comprising an AAV capsid variant described herein) may comprise a regulatory element (e.g., promoter), untranslated regions (UTR), a miR binding site a polyadenylation sequence (polyA), a filler or stuffer sequence, an intron, and/or a linker sequence, e.g., for enhancing payload expression.

In some embodiments, the viral genome components are selected and/or engineered for expression of a payload in a target tissue (e.g., a CNS tissue, a muscle tissue (e.g., heart), or DRG).

### Viral Genome Component: Inverted Terminal Repeats (ITRs)

In some embodiments, the AAV particle described herein (e.g., an AAV particle comprising an AAV capsid polypeptide, e.g., an AAV capsid variant described herein), comprises a viral genome (e.g., a viral genome encoding a SOD1 siRNA described herein), with at least one ITR and a payload region. In one embodiment, the viral genome has two ITRs. These two ITRs flank the payload region at the 5′ and 3′ ends. The ITRs function as origins of replication comprising recognition sites for replication. ITRs comprise sequence regions which can be complementary and symmetrically arranged. ITRs incorporated into viral genomes as described herein may be comprised of naturally occurring polynucleotide sequences or recombinantly derived polynucleotide sequences.

The ITRs may be derived from the same serotype as the capsid polypeptide, e.g., capsid variant, selected from any of the known serotypes, or a derivative thereof. The ITR may be of a different serotype than the capsid. In one embodiment, the AAV particle has more than one ITR. In a non-limiting example, the AAV particle has a viral genome comprising two ITRs. In one embodiment, the ITRs are of the same serotype as one another. In another embodiment, the ITRs are of different serotypes. Non-limiting examples include zero, one or both of the ITRs having the same serotype as the capsid. In one embodiment both ITRs of the viral genome of the AAV particle are AAV2 ITRs.

In some embodiments, the ITR sequence (e.g., 5′ ITR sequence) comprises the nucleotide sequence of SEQ ID NO: 126. In some embodiments, the ITR sequence (e.g., 5′ ITR sequence) comprises a nucleotide sequence having at least 90% identity, at least 95% identity, at least 98% identity, or at least 99% identity to SEQ ID NO: 126. In some embodiments, the ITR sequence (e.g., 5′ ITR sequence) has a nucleotide sequence with at 1, 2, 3, 4, 5, 6, 7, 8, 9, 10, 1-5, 1-10, 1-20, 10-20, 1-30, 10-30, or 20-30 nucleotide modifications (e.g., substitutions, insertions, and/or deletions) relative to SEQ ID NO: 126.

In some embodiments, the ITR sequence (e.g., 3′ ITR sequence) comprises the nucleotide sequence of SEQ ID NO: 130. In some embodiments, the ITR sequence (e.g., 3′ ITR sequence) comprises a nucleotide sequence having at least 90% identity, at least 95% identity, at least 98% identity, or at least 99% identity to SEQ ID NO: 130. In some embodiments, the ITR sequence (e.g., 3′ ITR sequence) has a nucleotide sequence with at 1, 2, 3, 4, 5, 6, 7, 8, 9, 10, 1-5, 1-10, 1-20, 10-20, 1-30, 10-30, or 20-30 nucleotide modifications (e.g., substitutions, insertions, and/or deletions) relative to SEQ ID NO: 130.

### Viral Genome Component: Promoters

In one embodiments, the payload region of the viral genome comprises at least one element to enhance the payload target specificity and expression (See e.g., Powell et al. Viral Expression Cassette Elements to Enhance Transgene Target Specificity and Expression in Gene Therapy, 2015; the contents of which are herein incorporated by reference in their entirety). Non-limiting examples of elements to enhance payload target specificity and expression include promoters, endogenous miRNAs, post-transcriptional regulatory elements (PREs), polyadenylation (PolyA) signal sequences and upstream enhancers (USEs), CMV enhancers and introns.

In some embodiments, an AAV particle comprising an AAV capsid variant described herein comprises a viral genome comprising a nucleic acid encoding a payload, wherein the nucleic acid is operably linked to a promoter. In some embodiments, the promoter is a species' specific promoter, an inducible promoter, tissue-specific, or cell cycle-specific (Parr et al., Nat. Med.3:1145-9 (1997); the contents of which are herein incorporated by reference in their entirety).

In some embodiments the promoter may be naturally occurring or non-naturally occurring. Non-limiting examples of promoters include those from viruses, plants, mammals, or humans. In some embodiments, the promoters may be those from human cells or systems. In some embodiments, the promoter may be truncated or mutated, e.g., a promoter variant.

In some embodiments, the promoter is deemed to be efficient when it drives expression of the payload encoded by the viral genome of the AAV particle.

In some embodiments, the promoter is a promoter deemed to be efficient when it drives expression in a cell being targeted.

In some embodiments, the promoter is a promoter having a tropism for a cell being targeted.

In some embodiments, the promoter drives expression of the payload (e.g., the SOD1 targeting siRNA described herein) for a period of time in targeted tissues. As a non-limiting example, the promoter is a selected for sustained expression of a payload in tissues and/or cells of the central or peripheral nervous system.

In some embodiments, the promoter is a ubiquitous promoter, e.g., capable of expression in multiple tissues. In some embodiments the promoter is a human elongation factor 1α-subunit (EF1α) promoter or a variant or fragment thereof, the cytomegalovirus (CMV) immediate-early enhancer and/or promoter, the chicken β-actin (CBA) promoter or a variant or fragment thereof and its derivative CAG, β glucuronidase (GUSB) promoter, phosphoglycerate kinase (PGK) promoter, or ubiquitin C (UBC) promoter. In some embodiments, the promoter is a cell or tissue specific promoter, e.g., capable of expression in tissues or cells of the central or peripheral nervous systems, regions within (e.g., frontal cortex), and/or sub-sets of cells therein (e.g., excitatory neurons). In some embodiments, the promoter is a cell-type specific promoter capable of expression a payload in excitatory neurons (e.g., glutamatergic), inhibitory neurons (e.g., GABA-ergic), neurons of the sympathetic or parasympathetic nervous system, sensory neurons, neurons of the dorsal root ganglia, motor neurons, or supportive cells of the nervous systems such as microglia, astrocytes, oligodendrocytes, and/or Schwann cells.

In some embodiments, the promoter is a liver promoter (e.g., hAAT, TBG), skeletal muscle specific promoter (e.g., desmin, MCK, C512), B cell promoter, monocyte promoter, leukocyte promoter, macrophage promoter, pancreatic acinar cell promoter, endothelial cell promoter, lung tissue promoter, and/or cardiac or cardiovascular promoter (e.g., αMHC, cTnT, and CMV-MLC2k).

In some embodiments, the promoter is a tissue-specific promoter for payload expression in a cell or tissue of the central nervous system. In some embodiments, the promoter is synapsin (Syn) promoter or a variant or fragment thereof, glutamate vesicular transporter (VGLUT) promoter, vesicular GABA transporter (VGAT) promoter, parvalbumin (PV) promoter, sodium channel Nav 1.8 promoter, tyrosine hydroxylase (TH) promoter, choline acetyltransferase (ChaT) promoter, methyl-CpG binding protein 2 (MeCP2) promoter, Ca²⁺/calmodulin-dependent protein kinase II (CaMKII) promoter, metabotropic glutamate receptor 2 (mGluR2) promoter, neurofilament light (NFL) or heavy (NFH) promoter, neuron-specific enolase (NSE) promoter, β-globin minigene nβ2 promoter, preproenkephalin (PPE) promoter, enkephalin (Enk) promoter, and excitatory amino acid transporter 2 (EAAT2) promoter. In some embodiments, the promoter is a cell-type specific promoter capable of expression in an astrocyte, e.g., a glial fibrillary acidic protein (GFAP) promoter and a EAAT2 promoter. In some embodiments, the promoter is a cell-type specific promoter capable of expression in an oligodendrocyte, e.g., a myelin basic protein (MBP) promoter.

In some embodiments, the promoter is a GFAP promoter. In some embodiments, the promoter is a synapsin (syn or syn1) promoter, or a fragment thereof.

In some embodiments, the promoter comprises an insulin promoter or a fragment thereof.

In certain embodiments, the promoter is a variant or fragment of any of the promoters described herein.

In some embodiments, the promoter is an H1 promoter. In some embodiments, the H1 promoter comprises the nucleotide sequence of SEQ ID NO: 83, or a nucleotide sequence at least 90%, at least 92%, at least 95%, at least 96%, at least 97%, at least 98%, or at least 99% identical thereto.

In some embodiments, the promoter comprises the nucleotide sequence of SEQ ID NO: 83, or a nucleotide sequence at least 90%, at least 92%, at least 95%, at least 96%, at least 97%, at least 98%, or at least 99% identical thereto.

In some embodiments, the promoter of the viral genome described herein (e.g., comprised within an AAV particle comprising an AAV capsid variant described herein) comprises an EF-1a promoter or a fragment or variant thereof, a chicken beta-actin (CBA) promoter or a fragment or variant thereof, a PGK promoter or a fragment or variant thereof, an insulin promoter or a fragment or variant thereof, a synapsin (SYN) promoter or a fragment or variant thereof, e.g., as provided in Table 7. In some embodiments, the promoter comprises the nucleotide sequence of any one of SEQ ID NOs: 4000-4026, a nucleotide sequence comprising at least one, two, or three but no more than four modifications (e.g., substitutions, insertions, and/or deletions) relative to the nucleotide sequence of SEQ ID NOs: 4000-4026, or a nucleotide sequence with at least 70% (e.g., 80, 85%, 90%, 95%, 96%, 97%, 98%, or 99%) sequence identity to any one of SEQ ID NOs: 4000-4026.

TABLE 7
Exemplary Promoter Sequences and Variants

| | | SEQ ID |
|---|---|---|
| Description | Sequences | NO: |
| EF1a | CGTGAGGCTCCGGTGCCCGTCAGTGGGCAGAGCGCACATCGCCCACAGTCCCCGAGA | 4000 |
| Promoter | AGTTGGGGGGAGGGGTCGGCAATTGAACCGGTGCCTAGAGAAGGTGGCGCGGGGTAA | |
| (intron | ACTGGGAAAGTGATGTCGTGTACTGGCTCCGCCTTTTTCCCGAGGGTGGGGGAGAAC | |
| underlined) | CGTATATAAGTGCAGTAGTCGCCGTGAACGTTCTTTTTCGCAACGGGTTTGCCGCCA | |
| | GAACACAGGTAAGTGCCGTGTGTGGTTCCCGCGGGCCTGGCCTCTTTACGGGTTATG | |
| | GCCCTTGCGTGCCTTGAATTACTTCCACCTGGCTGCAGTACGTGATTCTTGATCCCG | |
| | AGCTTCGGGTTGGAAGTGGGTGGGAGAGTTCGAGGCCTTGCGCTTAAGGAGCCCCTT | |
| | CGCCTCGTGCTTGAGTTGAGGCCTGGCCTGGGCGCTGGGGCCGCCGCGTGCGAATCT | |
| | GGTGGCACCTTCGCGCCTGTCTCGCTGCTTTCGATAAGTCTCTAGCCATTTAAAATT | |
| | TTTGATGACCTGCTGCGACGCTTTTTTTCTGGCAAGATAGTCTTGTAAATGCGGGCC | |
| | AAGATCTGCACACTGGTATTTCGGTTTTTGGGGCCGCGGGCGGCGACGGGGCCCGTG | |
| | CGTCCCAGCGCACATGTTCGGCGAGGCGGGGCCTGCGAGCGCGGCCACCGAGAATCG | |
| | GACGGGGGTAGTCTCAAGCTGGCCGGCCTGCTCTGGTGCCTGGCCTCGCGCCGCCGT | |
| | GTATCGCCCCGCCCTGGGCGGCAAGGCTGGCCCGGTCGGCACCAGTTGCGTGAGCGG | |
| | AAAGATGGCCGCTTCCCGGCCCTGCTGCAGGGAGCTCAAAATGGAGGACGCGGCGCT | |
| | CGGGAGAGCGGGCGGGTGAGTCACCCACACAAAGGAAAAGGGCCTTTCCGTCCTCAG | |
| | CCGTCGCTTCATGTGACTCCACGGAGTACCGGGCGCCGTCCAGGCACCTCGATTAGT | |
| | TCTCGAGCTTTTGGAGTACGTCGTCTTTAGGTTGGGGGGAGGGGTTTTATGCGATGG | |
| | AGTTTCCCCACACTGAGTGGGTGGAGACTGAAGTTAGGCCAGCTTGGCACTTGATGT | |
| | AATTCTCCTTGGAATTTGCCCTTTTTGAGTTTGGATCTTGGTTCATTCTCAAGCCTC | |
| | AGACAGTGGTTCAAAGTTTTTTTCTTCCATTTCAGGTGTCGTGA | |
| miniEF1a | GCCCGTCAGTGGGCAGAGCGCACATCGCCCACAGTCCCCGAGAAGTTGGGGGGAGGG | 4001 |
| | GTCGGCAATTGAACCGGTGCCTAGAGAAGGTGGCGCGGGGTAAACTGGGAAAGTGAT | |
| | GTCGTGTACTGGCTCCGCCTTTTTCCCGAGGGTGGGGGAGAACCGTATATAAGTGCA | |
| | GTAGTCGCCGTGAACGTTCTTTTTCGCAACGGGTTTGCCGCCAGAACACGCGTAAG | |
| Promoter | GCATG | |
| Variant 1 | | |
| Promoter | GGTGGAGAAGAGCATG | 4003 |
| Variant 2 | | |
| Promoter | GTCATCACTGAGGTGGAGAAGAGCATG | 4004 |
| Variant 3 | | |
| Promoter | CGTGAG | |
| Variant 4 | | |
| Promoter | GT | |
| Variant 5 | | |
| Promoter | GCTCCGGT | |
| Variant 6 | | |
| Promoter | GCCCGTCAGTGGGCAGAGCGCACATCGCCCACAGTCCCCGAGAAGTTGGGGGGAGGG | 4008 |
| Variant 19 | GTCGGCAATTGAACCGGTGCCTAGAGAAGGTGGCGCGGGGTAAACTGGGAAAGTGAT | |
| | GTCGTGTACTGGCTCCGCCTTTTTCCCGAGGGTGGGGGAGAACCGTATATAAGTGCA | |
| | GTAGTCGCCGTGAACGTTCTTTTTCGCAACGGGTTTGCCGCCAGAACACAG | |
| Promoter | GCCCGTCAGTGGGCAGAGCGCACATCGCCCACAGTCCCCGAGAAGTTGGGGGGAGGG | 4009 |
| Variant 20 | GTCGGCAATTGAACCGGTGCCTAGAGAAGGTGGCGCGGGGTAAACTGGGAAAGTGAT | |
| | GTCGTGTACTGGCTCCGCCTTTTTCCCGAGGGTGGGGGAGAACCGTATATAAGTGCA | |
| | GTAGTCGCCGTGAACGTTCTTTTTCGCAACGGGTTTGCCGCCAGAACACGC | |
| Promoter | GTAAG | |
| Variant 7 | | |
| Promoter | GTGCCCGTCAGTGGGCAGAGCGCACATCGCCCACAGTCCCCGAGAAGTTGGGGGGAG | 4011 |
| Variant 8 | GGGTCGGCAATTGAACCGGTGCCTAGAGAAGGTGGCGCGGGGTAAACTGGGAAAGTG | |
| | ATGTCGTGTACTGGCTCCGCCTTTTTCCCGAGGGTGGGGGAGAACCGTATATAAGTG | |
| | CAGTAGTCGCCGTGAACGTTCTTTTTCGCAACGGGTTTGCCGCCAGAACACGCGTAA | |
| | G | |
| Promoter | GCTCCGGTGCCCGTCAGTGGGCAGAGCGCACATCGCCCACAGTCCCCGAGAAGTTGG | 4012 |
| Variant 9 | GGGGAGGGGTCGGCAATTGAACCGGTGCCTAGAGAAGGTGGCGCGGGGTAAACTGGG | |
| | AAAGTGATGTCGTGTACTGGCTCCGCCTTTTTCCCGAGGGTGGGGGAGAACCGTATA | |
| | TAAGTGCAGTAGTCGCCGTGAACGTTCTTTTTCGCAACGGGTTTGCCGCCAGAACAC | |
| | GCGTAAG | |
| Promoter | CGTGAGGCTCCGGTGCCCGTCAGTGGGCAGAGCGCACATCGCCCACAGTCCCCGAGA | 4013 |
| Variant 10 | AGTTGGGGGGAGGGGTCGGCAATTGAACCGGTGCCTAGAGAAGGTGGCGCGGGGTAA | |
| | ACTGGGAAAGTGATGTCGTGTACTGGCTCCGCCTTTTTCCCGAGGGTGGGGGAGAAC | |
| | CGTATATAAGTGCAGTAGTCGCCGTGAACGTTCTTTTTCGCAACGGGTTTGCCGCCA | |
| | GAACACGCGTAAG | |
| Promoter | CGTGAGGCTCCGGTGCCCGTCAGTGGGCAGAGCGCACATCGCCCACAGTCCCCGAGA | 4014 |
| Variant 11 | AGTTGGGGGGAGGGGTCGGCAATTGAACCGGTGCCTAGAGAAGGTGGCGCGGGGTAA | |
| | ACTGGGAAAGTGATGTCGTGTACTGGCTCCGCCTTTTTCCCGAGGGTGGGGGAGAAC | |
| | CGTATATAAGTGCAGTAGTCGCCGTGAACGTTCTTTTTCGCAACGGGTTTGCCGCCA | |
| | GAACACAG | |
| Promoter | GCATGCGTGAGGCTCCGGTGCCCGTCAGTGGGCAGAGCGCACATCGCCCACAGTCCC | 4015 |
| Variant 12 | CGAGAAGTTGGGGGGAGGGGTCGGCAATTGAACCGGTGCCTAGAGAAGGTGGCGCGG | |
| | GGTAAACTGGGAAAGTGATGTCGTGTACTGGCTCCGCCTTTTTCCCGAGGGTGGGGG | |
| | AGAACCGTATATAAGTGCAGTAGTCGCCGTGAACGTTCTTTTTCGCAACGGGTTTGC | |
| | CGCCAGAACACGCGTAAG | |
| Promoter | GCATGCGTGAGGCTCCGGTGCCCGTCAGTGGGCAGAGCGCACATCGCCCACAGTCCC | 4016 |
| Variant 13 | CGAGAAGTTGGGGGGAGGGGTCGGCAATTGAACCGGTGCCTAGAGAAGGTGGCGCGG | |
| | GGTAAACTGGGAAAGTGATGTCGTGTACTGGCTCCGCCTTTTTCCCGAGGGTGGGGG | |
| | AGAACCGTATATAAGTGCAGTAGTCGCCGTGAACGTTCTTTTTCGCAACGGGTTTGC | |
| | CGCCAGAACACAG | |
| Promoter | GGTGGAGAAGAGCATGCGTGAGGCTCCGGTGCCCGTCAGTGGGCAGAGCGCACATCG | 4017 |
| Variant 14 | CCCACAGTCCCCGAGAAGTTGGGGGGAGGGGTCGGCAATTGAACCGGTGCCTAGAGA | |
| | AGGTGGCGCGGGGTAAACTGGGAAAGTGATGTCGTGTACTGGCTCCGCCTTTTTCCC | |
| | GAGGGTGGGGGAGAACCGTATATAAGTGCAGTAGTCGCCGTGAACGTTCTTTTTCGC | |
| | AACGGGTTTGCCGCCAGAACACGCGTAAG | |
| Promoter | GGTGGAGAAGAGCATGCGTGAGGCTCCGGTGCCCGTCAGTGGGCAGAGCGCACATCG | 4018 |
| Variant 15 | CCCACAGTCCCCGAGAAGTTGGGGGGAGGGGTCGGCAATTGAACCGGTGCCTAGAGA | |
| | AGGTGGCGCGGGGTAAACTGGGAAAGTGATGTCGTGTACTGGCTCCGCCTTTTTCCC | |
| | GAGGGTGGGGGAGAACCGTATATAAGTGCAGTAGTCGCCGTGAACGTTCTTTTTCGC | |
| | AACGGGTTTGCCGCCAGAACACAG | |
| Promoter | GTCATCACTGAGGTGGAGAAGAGCATGCGTGAGGCTCCGGTGCCCGTCAGTGGGCAG | 4019 |
| Variant 16 | AGCGCACATCGCCCACAGTCCCCGAGAAGTTGGGGGGAGGGGTCGGCAATTGAACCG | |
| | GTGCCTAGAGAAGGTGGCGCGGGGTAAACTGGGAAAGTGATGTCGTGTACTGGCTCC | |
| | GCCTTTTTCCCGAGGGTGGGGGAGAACCGTATATAAGTGCAGTAGTCGCCGTGAACG | |
| | TTCTTTTTCGCAACGGGTTTGCCGCCAGAACACGCGTAAG | |
| Promoter | GTCATCACTGAGGTGGAGAAGAGCATGCGTGAGGCTCCGGTGCCCGTCAGTGGGCAG | 4020 |
| Variant 18 | AGCGCACATCGCCCACAGTCCCCGAGAAGTTGGGGGGAGGGGTCGGCAATTGAACCG | |
| | GTGCCTAGAGAAGGTGGCGCGGGGTAAACTGGGAAAGTGATGTCGTGTACTGGCTCC | |
| | GCCTTTTTCCCGAGGGTGGGGGAGAACCGTATATAAGTGCAGTAGTCGCCGTGAACG | |
| | TTCTTTTTCGCAACGGGTTTGCCGCCAGAACACAG | |
| Chicken beta- | CGACATTGATTATTGACTAGTTATTAATAGTAATCAATTACGGGGTCATTAGTTCAT | 4021 |
| actin (CBA) | AGCCCATATATGGAGTTCCGCGTTACATAACTTACGGTAAATGGCCCGCCTGGCTGA | |
| promoter | CCGCCCAACGACCCCCGCCCATTGACGTCAATAATGACGTATGTTCCCATAGTAACG | |
| | CCAATAGGGACTTTCCATTGACGTCAATGGGTGGAGTATTTACGGTAAACTGCCCAC | |
| | TTGGCAGTACATCAAGTGTATCATATGCCAAGTACGCCCCCTATTGACGTCAATGAC | |
| | GGTAAATGGCCCGCCTGGCATTATGCCCAGTACATGACCTTATGGGACTTTCCTACT | |
| | TGGCAGTACATCTACGTATTAGTCATCGCTATTACCATGTCGAGGCCACGTTCTGCT | |
| | TCACTCTCCCCATCTCCCCCCCCTCCCCACCCCCAATTTTGTATTTATTTATTTTTT | |
| | AATTATTTTGTGCAGCGATGGGGGCGGGGGGGGGGGGCGCGCGCCAGGCGGGGGGGG | |
| | GCGGGGCGAGGGGGGGGGCGGGGCGAGGCGGAGAGGTGCGGCGGCAGCCAATCAGAG | |
| | CGGCGCGCTCCGAAAGTTTCCTTTTATGGCGAGGCGGCGGCGGCGGCGGCCCTATAA | |
| | AAAGCGAAGCGCGCGGCGGGCGGGAGC | |
| Mini-CBA | CCACGTTCTGCTTCACTCTCCCCATCTCCCCCCCCTCCCCACCCCCAATTTTGTATT | 4022 |
| promoter | TATTTATTTTTTAATTATTTTGTGCAGCGATGGGGGCGGGGGGGGGGGGCGCGCGCC | |
| | AGGCGGGGCGGGGGGGGCGAGGGGCGGGGCGGGGCGAGGCGGAGAGGTGCGGCGGC | |
| | AGCCAATCAGAGCGGCGCGCTCCGAAAGTTTCCTTTTATGGCGAGGCGGCGGCGGCG | |
| | GCGGCCCTATAAAAAGCGAAGCGCGCGGCGGG | |
| PGK promoter | GGGTAGGGGAGGCGCTTTTCCCAAGGCAGTCTGGAGCATGCGCTTTAGCAGCCCCGC | 4023 |
| | TGGGCACTTGGCGCTACACAAGTGGCCTCTGGCCTCGCACACATTCCACATCCACCG | |
| | GTAGGCGCCAACCGGCTCCGTTCTTTGGTGGCCCCTTCGCGCCACCTTCTACTCCTC | |
| | CCCTAGTCAGGAAGTTCCCCCCCGCCCCGCAGCTCGCGTCGTGCAGGACGTGACAAA | |
| | TGGAAGTAGCACGTCTCACTAGTCTCGTGCAGATGGACAGCACCGCTGAGCAATGGA | |
| | AGCGGGTAGGCCTTTGGGGCAGCGGCCAATAGCAGCTTTGCTCCTTCGCTTTCTGGG | |
| | CTCAGAGGCTGGGAAGGGGTGGGTCCGGGGGCGGGCTCAGGGGGGGGCTCAGGGGCG | |
| | GGGGGGCGCCCGAAGGTCCTCCGGAGGCCCGGCATTCTGCACGCTTCAAAAGCGCA | |
| | CGTCTGCCGCGCTGTTCTCCTCTTCCTCATCTCCGGGCCTTTCG | |
| Insulin | CCCTAATGGGCCAGGCGGCAGGGGTTGAGAGGTAGGGGAGATGGGCTCTGAGACTAT | 4024 |
| promoter | AAAGCCAGCGGGGGCCCAGCAGCCCTC | |
| fragment | | |
| Human | CGCGTGGGGTTATTTCTCTACTTTCGTGTCTCTGAGTGTGCTTCCAGTGCCCCCCTC | 4025 |
| synapsin | CCCCCAAAAAATGCCTTCTGAGTTGAATATCAACACTACAAACCTAGTATCTGCAGA | |
| (SYN) | GGGCCCTGCGTATGAGTGCAAGTGGGTTTTAGGACCAGGATGAGGCGGGGTGGGGGT | |
| promoter | GCCTACCTGACGACCGACCCCGACCCACTGGACAAGCACCCAACCCCCATTCCCCAA | |
| (long) | ATTGCGCATCCCCTATCAGAGAGGGGGAGGGGAAACAGGATGCGGCGAGGCGCGTGC | |
| | GCACTGCCAGCTTCAGCACCGCGGACAGTGCCTTCGCCCCCGCCTGGCGGCGCGCGC | |
| | CACCGCCGCCTCAGCACTGAAGGCGCGCTGACGTCACTCGCCGGTCCCCCGCAAACT | |
| | CCCCTTCCCGGCCACCTTGGTCGCGTCCGCGCCGCCGCCGGCCCAGCCGGACCGCAC | |
| | CACGCGAGGCGCGAGATAGGGGGGCACGGGCGCGACCATCTGCGCTGCGGCGCCGGC | |
| | GACTCAGCGCTGCCTCAGTCTGCGGTGGGCAGCGGAGGAGTCGTGTCGTGCCTGAGA | |
| | GCGCAGCTGTGCTCCTGGGCACCGCGCAGTCCGCCCCCGCGGCTCCTGGCCAGACCA | |
| | CCCCTAGGACCCCCTGCCCCAAGTCGCAGCC | |
| Human SYN | TAGTATCTGCAGAGGGCCCTGCGTATGAGTGCAAGTGGGTTTTAGGACCAGGATGAG | 4026 |
| promoter | GCGGGGTGGGGGTGCCTACCTGACGACCGACCCCGACCCACTGGACAAGCACCCAAC | |
| (short) | CCCCATTCCCCAAATTGCGCATCCCCTATCAGAGAGGGGGAGGGGAAACAGGATGCG | |
| | GCGAGGCGCGTGCGCACTGCCAGCTTCAGCACCGCGGACAGTGCCTTCGCCCCCGCC | |
| | TGGCGGCGCGCGCCACCGCCGCCTCAGCACTGAAGGCGCGCTGACGTCACTCGCCGG | |
| | TCCCCCGCAAACTCCCCTTCCCGGCCACCTTGGTCGCGTCCGCGCCGCCGCCGGCCC | |
| | AGCCGGACCGCACCACGCGAGGCGCGAGATAGGGGGGCACGGGCGCGACCATCTGCG | |
| | CTGCGGCGCCGGCGACTCAGCGCTGCCTCAGTCTGCGGTGGGCAGCGGAGGAGTCGT | |
| | GTCGTGCCTGAGAGCGCAGCTGTGCTCCTGGGCACCGCGCAGTCCGCCCCCGCGGCT | |
| | CCTGGCCAGACCACCCCTAGGACCCCCTGCCCCAAGTCGCAGCC | |

### Viral Genome Component: Untranslated Regions (UTRs)

In some embodiments, wild type untranslated regions (UTRs) of a gene are transcribed but not translated. Generally, the 5′ UTR starts at the transcription start site and ends at the start codon and the 3′ UTR starts immediately following the stop codon and continues until the termination signal for transcription.

Features typically found in abundantly expressed genes of specific target organs (e.g., CNS tissue, muscle, or DRG) may be engineered into UTRs to enhance stability and protein production. As a non-limiting example, a 5′ UTR from mRNA normally expressed in the brain (e.g., huntingtin) may be used in the viral genomes of the AAV particles described herein to enhance expression in neuronal cells or other cells of the central nervous system.

While not wishing to be bound by theory, wild-type 5′ untranslated regions (UTRs) include features which play roles in translation initiation. Kozak sequences, which are commonly known to be involved in the process by which the ribosome initiates translation of many genes, are usually included in 5′ UTRs. Kozak sequences have the consensus CCR (A/G) CCAUGG, where R is a purine (adenine or guanine) three bases upstream of the start codon (ATG), which is followed by another ‘G’.

In one embodiment, the 5′UTR in the viral genome includes a Kozak sequence.

In one embodiment, the 5′UTR in the viral genome does not include a Kozak sequence.

While not wishing to be bound by theory, wild-type 3′ UTRs are known to have stretches of Adenosines and Uridines embedded therein. These AU rich signatures are particularly prevalent in genes with high rates of turnover. Based on their sequence features and functional properties, the AU rich elements (AREs) can be separated into three classes (Chen et al, 1995, the contents of which are herein incorporated by reference in its entirety): Class I AREs, such as, but not limited to, c-Myc and MyoD, contain several dispersed copies of an AUUUA motif within U-rich regions. Class II AREs, such as, but not limited to, GM-CSF and TNF-α, possess two or more overlapping UUAUUUA (U/A) (U/A) nonamers. Class III ARES, such as, but not limited to, c-Jun and Myogenin, are less well defined. These U rich regions do not contain an AUUUA motif. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of a polynucleotide. When engineering specific polynucleotides, e.g., payload regions of viral genomes, one or more copies of an ARE can be introduced to make polynucleotides less stable and thereby curtail translation and decrease production of the resultant protein. Likewise, AREs can be identified and removed or mutated to increase the intracellular stability and thus increase translation and production of the resultant protein.

In some embodiments, the 3′ UTR of the viral genome may include an oligo (dT) sequence for templated addition of a poly-A tail.

In some embodiments, the viral genome may include at least one miRNA secd, binding site or full sequence. microRNAs (or miRNA or miR) are 19-25 nucleotide noncoding RNAs that bind to the sites of nucleic acid targets and down-regulate gene expression either by reducing nucleic acid molecule stability or by inhibiting translation. In some embodiments, a microRNA sequence comprises a seed region, e.g., a sequence in the region of positions 2-8 of the mature microRNA, which has Watson-Crick sequence fully or partially complementarity to the miRNA target sequence of the nucleic acid.

In some embodiments, the viral genome may be engineered to include, alter or remove at least one miRNA binding site, full sequence or seed region.

Any UTR from any gene known in the art may be incorporated into the viral genome of the AAV particle. These UTRs, or portions thereof, may be placed in the same orientation as in the gene from which they were selected or they may be altered in orientation or location. In one embodiment, the UTR used in the viral genome of the AAV particle may be inverted, shortened, lengthened, made with one or more other 5′ UTRs or 3′ UTRs known in the art. As used herein, the term “altered” as it relates to a UTR, means that the UTR has been changed in some way in relation to a reference sequence. For example, a 3′ or 5′ UTR may be altered relative to a wild type or native UTR by the change in orientation or location as taught above or may be altered by the inclusion of additional nucleotides, deletion of nucleotides, swapping or transposition of nucleotides.

In some embodiments, the viral genome of the AAV particle comprises at least one artificial UTR which is not a variant of a wild type UTR.

In some embodiments, the viral genome of the AAV particle comprises UTRs which have been selected from a family of transcripts whose proteins share a common function, structure, feature or property.

### Viral Genome Component: Polyadenylation Sequence

The viral genome encoding a SOD1 siRNA described herein may comprise at least one polyadenylation sequence. In some embodiments, the viral genome of the AAV particle (e.g., an AAV particle comprising an AAV capsid polypeptide, e.g., an AAV capsid variant, described herein) comprises a polyadenylation sequence between the 3′ end of the payload encoding region and the 5′ end of the 3′ITR.

In some embodiments, the AAV particle comprises a nucleic acid sequence encoding an siRNA molecule may be located upstream of the polyadenylation sequence in an expression vector. Further, the AAV particle comprises a nucleic acid sequence encoding an siRNA molecule may be located downstream of a promoter such as, but not limited to, CMV, U6, CAG, CBA or a CBA promoter with a SV40 intron or a human beta globin intron in an expression vector.

In some embodiments, the AAV particle comprises a rabbit globin polyadenylation (polyA) signal sequence (rBGpA).

In some embodiments, the AAV particle comprises a human growth hormone (hGH) polyadenylation (polyA) signal sequence.

In some embodiments, the polyadenylation signal sequence comprises the nucleotide sequence of SEQ ID NO: 129 or 4027, a nucleotide sequence comprising at least one, two, or three but no more than four modifications (e.g., substitutions, insertions, and/or deletions) relative to the nucleotide sequence of SEQ ID NO: 129 or 4027, or a nucleotide sequence with at least 70% (e.g., 80%, 85%, 90%, 95%, 96%, 97%, 98%, or 99%) sequence identity to SEQ ID NO: 129 or 4027.

In some embodiments, the polyadenylation signal sequence comprises the nucleotide sequence of SEQ ID NO: 129, a nucleotide sequence comprising at least one, two, or three but no more than four modifications (e.g., substitutions, insertions, and/or deletions) relative to the nucleotide sequence of SEQ ID NO: 129, or a nucleotide sequence with at least 70%, at least 80%, at least 85%, at least 90%, at least 95%, at least 96%, at least 97%, at least 98%, or at least 99% sequence identity to SEQ ID NO: 129.

TABLE 8
Exemplary Poly A sequences

| | | SEQ ID |
|---|---|---|
| Description | Sequence | NO: |
| rBGpA polyA | GATCTTTTTCCCTCTGCCAAAAATTATGGGGACATCATGAAGCCCCTTGAGCATC | 129 |
| signal | TGACTTCTGGCTAATAAAGGAAATTTATTTTCATTGCAATAGTGTGTTGGAATTT | |
| sequence | TTTGTGTCTCTCACTCG | |
| Human growth | GGGTGGCATCCCTGTGACCCCTCCCCAGTGCCTCTCCTGGCCCTGGAAGTTGCCA | 4027 |
| hormone | CTCCAGTGCCCACCAGCCTTGTCCTAATAAAATTAAGTTGCATCATTTTGTCTGA | |
| (hGH) polyA | CTAGGTGTCCTTCTATAATATTATGGGGTGGAGGGGGGTGGTATGGAGCAAGGGG | |
| signal | CAAGTTGGGAAGACAACCTGTAGGGCCTGCGGGGTCTATTGGGAACCAAGCTGGA | |
| sequence | GTGCAGTGGCACAATCTTGGCTCACTGCAATCTCCGCCTCCTGGGTTCAAGCGAT | |
| | TCTCCTGCCTCAGCCTCCCGAGTTGTTGGGATTCCAGGCATGCATGACCAGGCTC | |
| | AGCTAATTTTTGTTTTTTTGGTAGAGACGGGGTTTCACCATATTGGCCAGGCTGG | |
| | TCTCCAACTCCTAATCTCAGGTGATCTACCCACCTTGGCCTCCCAAATTGCTGGG | |
| | ATTACAGGCGTGAACCACTGCTCCCTTCCCTGTCCTT | |

### Viral Genome Component: Exons and Introns

In some embodiments, the viral genome encoding a SOD1 targeting siRNA described herein, comprises at least one element to enhance the payload target specificity and expression (Sec e.g., Powell et al. Viral Expression Cassette Elements to Enhance Transgene Target Specificity and Expression in Gene Therapy, Discov. Med, 2015, 19 (102): 49-57; the contents of which are herein incorporated by reference in their entirety) such as an exon and/or intron. Non-limiting examples of introns include, MVM (67-97 bps), F.IX truncated intron 1 (300 bps), β-globin SD/immunoglobulin heavy chain splice acceptor (250 bps), adenovirus splice donor/immunoglobin splice acceptor (500 bps), SV40 late splice donor/splice acceptor (19S/16S) (180 bps) and hybrid adenovirus splice donor/IgG splice acceptor (230 bps). In one embodiment, the intron is a beta globulin intron.

In one embodiment, the intron or intron portion may be 100-500 nucleotides in length. The intron may have a length of 80, 90, 100, 110, 120, 130, 140, 150, 160, 170, 171, 172, 173, 174, 175, 176, 177, 178, 179, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or 500 nucleotides. The intron may have a length between 80-100, 80-120, 80-140, 80-160, 80-180, 80-200, 80-250, 80-300, 80-350, 80-400, 80-450, 80-500, 200-300, 200-400, 200-500, 300-400, 300-500, or 400-500 nucleotides.

In some embodiments, the viral genome encoding a SOD1 targeting siRNA described herein comprises an intron comprising the nucleotide sequence of SEQ ID NO: 4044, a nucleotide sequence comprising at least one, two, or three but no more than four modifications (e.g., substitutions, insertions, and/or deletions), relative to the nucleotide sequence of SEQ ID NO: 4044, or a nucleotide sequence with at least 70% (e.g., 80, 85%, 90%, 95%, 96%, 97%, 98%, or 99%) sequence identity to SEQ ID NO: 4044.

In some embodiments, the intron is positioned 3′ relative to a 5′ITR and/or promoter, and 5′ relative to a nucleotide sequence encoding a modulatory polynucleotide (e.g., a SOD1 targeting RNA agent, e.g., a siRNA), a poly A signal sequence, and/or a 3′ITR.

In some embodiments, the viral genome encoding a SOD1 targeting siRNA described herein comprises one or more exons (e.g., 1, 2, 3, 4, 5, 6, 7, or 8 or more exons). In some embodiments, the viral genome comprises a first exon, a second exon, or both a first and a second exon.

In some embodiments, the first or second exon comprises the nucleotide sequence of any one of SEQ ID NO: 4045-4048, a nucleotide sequence comprising at least one, two, or three but no more than four modifications, e.g., substitutions, insertions, or deletions, relative to the nucleotide sequence of any one of SEQ ID NO: 4045-4048, or a nucleotide sequence with at least 80% (e.g., 85%, 90%, 95%, 96%, 97%, 98%, or 99%) sequence identity to any one of SEQ ID NO: 4045-4048. In some embodiments, the viral genome comprises both a first exon and a second exon, wherein: (a) the first exon comprises the nucleotide sequence of SEQ ID NO: 4045 or 4046, a nucleotide sequence comprising at least one, two, or three but no more than four modifications, e.g., substitutions, insertions, or deletions, relative to the nucleotide sequence of SEQ ID NO: 4045 or 4046, or a nucleotide sequence with at least 80% (e.g., 85%, 90%, 95%, 96%, 97%, 98%, or 99%) sequence identity to SEQ ID NO: 4045 or 4046; and (b) the second exon comprises the nucleotide sequence of SEQ ID NO: 4047 or 4048, a nucleotide sequence comprising at least one, two, or three but no more than four modifications, e.g., substitutions, insertions, or deletions, relative to the nucleotide sequence of SEQ ID NO: 4047 or 4048, or a nucleotide sequence with at least 80% (e.g., 85%, 90%, 95%, 96%, 97%, 98%, or 99%) sequence identity to SEQ ID NO: 4047 or 4048.

In some embodiments, one or more or all exons are codon optimized. In some embodiments, one or more or all of the CpG sequences in the one or more or all exons are depleted (e.g., altered to remove CpG sequences, such as by substitution, insertion, or deletion).

In some embodiments, the viral genome comprises a first exon, an intron (e.g., an intron comprising the sequence of SEQ ID NO: 4044 or a variant thereof), and a second exon (referred to herein as an “exon-intron-exon cassette”).

In some embodiments, the viral genome comprises a first exon, an intron (e.g., an intron comprising the sequence of SEQ ID NO: 4044 or a variant thereof), and a second exon, wherein the intron is located 3′ relative to the first exon and 5′ relative to the second exon.

In some embodiments, the exon-intron-exon cassette comprises the nucleotide sequence of SEQ ID NO: 4042 or 4043, a nucleotide sequence comprising at least one, two, or three but no more than four modifications, e.g., substitutions, insertions, or deletions, relative to the nucleotide sequence of SEQ ID NO: 4042 or 4043, or a nucleotide sequence with at least 80% (e.g., 85%, 90%, 95%, 96%, 97%, 98%, or 99%) sequence identity to SEQ ID NO: 4042 or 4043.

In some embodiments, the exon-intron-exon cassette is positioned 3′ relative to the 5′ITR and/or promoter, and 5′ relative to the nucleotide sequence encoding the modulatory polynucleotide (e.g., a SOD1 targeting RNA agent, e.g., a siRNA), poly A signal sequence, and/or 3′ITR.

In some embodiments, the nucleotide sequence encoding the modulatory polynucleotide (e.g., a SOD1 targeting RNA agent, e.g., a siRNA) is located within (e.g., inserted into) the intron of the exon-intron-exon cassette.

In some embodiments, the nucleotide sequence encoding the modulatory polynucleotide (e.g., a SOD1 targeting RNA agent, e.g., a siRNA) replaces all or a portion of the intron in the exon-intron-exon cassette.

TABLE 9
Exemplary exon, intron, and exon-intron-exon cassette sequences

| | | SEQ ID |
|---|---|---|
| Description | Sequence | NO: |
| Human beta | TCAGATCGCCTGGAGACGCCATCCACGCTGTTTTGACCTCCATAGAAGACACCGG | 4042 |
| globulin (hβG) | GACCGATCCAGCCTCCGCGGATTCGAATCCCGGCCGGGAACGGTGCATTGGAACG | |
| exon-intron- | CGGATTCCCCGTGCCAAGAGTGACGTAAGTACCGCCTATAGAGTCTATAGGCCCA | |
| exon cassette | CAAAAAATGCTTTCTTCTTTTAATATACTTTTTTGTTTATCTTATTTCTAATACT | |
| [CpG | TTCCCTAATCTCTTTCTTTCAGGGCAATAATGATACAATGTATCATGCCTCTTTG | |
| underlined] | CACCATTCTAAAGAATAACAGTGATAATTTCTGGGTTAAGGCAATAGCAATATTT | |
| | CTGCATATAAATATTTCTGCATATAAATTGTAACTGATGTAAGAGGTTTCATATT | |
| | GCTAATAGCAGCTACAATCCAGCTACCATTCTGCTTTTATTTTATGGTTGGGATA | |
| | AGGCTGGATTATTCTGAGTCCAAGCTAGGCCCTTTTGCTAATCATGTTCATACCT | |
| | CTTATCTTCCTCCCACAGCTCCTGGGCAACGTGCTGGTCTGTGTGCTGGCCCATC | |
| | ACTTTGGCAAAGAATT | |
| hβG exon- | TCAGATCACCTGGAGACACCATCCACACTGTTTTGACCTCCATAGAAGACACCAG | 4043 |
| intron-exon | GACCAATCCAGCCTCCACAGATTCCAATCCCAGCCAGGAACAGTGCATTGGAACA | |
| cassette [CpG | CAGATTCCCCTTGCCAAGAGTGACGTAAGTACCGCCTATAGAGTCTATAGGCCCA | |
| depleted] | CAAAAAATGCTTTCTTCTTTTAATATACTTTTTTGTTTATCTTATTTCTAATACT | |
| | TTCCCTAATCTCTTTCTTTCAGGGCAATAATGATACAATGTATCATGCCTCTTTG | |
| | CACCATTCTAAAGAATAACAGTGATAATTTCTGGGTTAAGGCAATAGCAATATTT | |
| | CTGCATATAAATATTTCTGCATATAAATTGTAACTGATGTAAGAGGTTTCATATT | |
| | GCTAATAGCAGCTACAATCCAGCTACCATTCTGCTTTTATTTTATGGTTGGGATA | |
| | AGGCTGGATTATTCTGAGTCCAAGCTAGGCCCTTTTGCTAATCATGTTCATACCT | |
| | CTTATCTTCCTCCCACAGCTCCTGGGCAACCTGCTGGTCTGTGTGCTGGCCCATC | |
| | ACTTTGGCAAAGAATT | |
| hβG intron | GTAAGTACCGCCTATAGAGTCTATAGGCCCACAAAAAATGCTTTCTTCTTTTAAT | 4044 |
| | ATACTTTTTTGTTTATCTTATTTCTAATACTTTCCCTAATCTCTTTCTTTCAGGG | |
| | CAATAATGATACAATGTATCATGCCTCTTTGCACCATTCTAAAGAATAACAGTGA | |
| | TAATTTCTGGGTTAAGGCAATAGCAATATTTCTGCATATAAATATTTCTGCATAT | |
| | AAATTGTAACTGATGTAAGAGGTTTCATATTGCTAATAGCAGCTACAATCCAGCT | |
| | ACCATTCTGCTTTTATTTTATGGTTGGGATAAGGCTGGATTATTCTGAGTCCAAG | |
| | CTAGGCCCTTTTGCTAATCATGTTCATACCTCTTATCTTCCTCCCACAG | |
| hβG exon (first | TCAGATCGCCTGGAGACGCCATCCACGCTGTTTTGACCTCCATAGAAGACACCGG | 4045 |
| exon) [CpG | GACCGATCCAGCCTCCGCGGATTCGAATCCCGGCCGGGAACGGTGCATTGGAACG | |
| underlined] | CGGATTCCCCGTGCCAAGAGTGAC | |
| hβG exon (first | TCAGATCACCTGGAGACACCATCCACACTGTTTTGACCTCCATAGAAGACACCAG | 4046 |
| exon) [CpG | GACCAATCCAGCCTCCACAGATTCCAATCCCAGCCAGGAACAGTGCATTGGAACA | |
| depleted] | CAGATTCCCCTTGCCAAGAGTGAC | |
| hβG exon | CTCCTGGGCAACGTGCTGGTCTGTGTGCTGGCCCATCACTTTGGCAAAGAATT | 4047 |
| (second exon) | | |
| [CpG | | |
| underlined] | | |
| hβG exon | CTCCTGGGCAACCTGCTGGTCTGTGTGCTGGCCCATCACTTTGGCAAAGAATT | 4048 |
| (second exon) | | |
| [CpG depleted] | | |

In some embodiments, the encoded siRNA molecule may be located downstream of a promoter in an expression vector such as, but not limited to, CMV, U6, H1, CBA, CAG, or a CBA promoter with an intron such as SV40 or others known in the art. Further, the encoded siRNA molecule may also be located upstream of the polyadenylation sequence in an expression vector.

In some embodiments, the nucleotide sequence encoding the siRNA may be located within an intron (e.g., inserted into an intron), or replace all or a portion of an intron sequence.

In some embodiments, the nucleotide sequence encoding the siRNA may be located within an intron (e.g., inserted into an intron), or replace all or a portion of an intron sequence, of an exon-intron-exon cassette (e.g., the exon-intron-exon cassette of SEQ ID NO: 4042 or 4043).

### Viral Genome Component: Stuffer or Filler Sequence

In some embodiments, the viral genome encoding a SOD1 targeting siRNA described herein, comprises at least one element to improve packaging efficiency and expression, such as a stuffer or filler sequence. Non-limiting examples of a stuffer sequence includes albumin and/or alpha-1 antitrypsin. Any known viral, mammalian, or plant sequence may be manipulated for use as a stuffer sequence.

In one embodiment, the stuffer or filler sequence may be from about 100-3500 nucleotides in length. The stuffer sequence may have a length of about 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900 or 3000 nucleotides.

In some embodiments, the filler sequence may comprise a region or a portion of a lentivirus. In some embodiments, the filler sequence comprises the nucleotide sequence of SEQ ID NO: 82, or a nucleotide sequence at least 70%, 75%, 80%, 85%, 90%, 92%, 95%, 96%, 97%, 98%, or 99% identical thereto. In some embodiments, the filler sequence comprises the nucleotide sequence of SEQ ID NO: 82, or a nucleotide sequence at least 90%, 92%, 95%, 96%, 97%, 98%, or 99% identical thereto. In some embodiments, the filler sequence comprises the nucleotide sequence of SEQ ID NO: 82, or a nucleotide sequence at least 95%, 96%, 97%, 98%, or 99% identical thereto.

In some embodiments, the filler sequence may comprise a region or a portion of the albumin gene. In certain embodiments, the filler sequence may comprise a region or a portion of the human albumin gene (NCBI Reference Sequence: NG_009291.1).

### Viral Genome Component: MiRNA

In some embodiments, the viral genome comprises a sequence encoding a miRNA to reduce the expression of the payload in a tissue or cell, e.g., the DRG (dorsal root ganglion), or neurons of other ganglia, such as those of the sympathetic or parasympathetic nervous system. In some embodiments, a miRNA, e.g., a miR183, a miR182, and/or miR96, may be encoded in the viral genome to modulate, e.g., reduce the expression, of the viral genome in a DRG neuron. As another non-limiting example, a miR-122 miRNA may be encoded in the viral genome to modulate, e.g., reduce, the expression of the viral genome in the liver. In some embodiments, a miRNA, e.g., a miR-142-3p, may be encoded in the viral genome to modulate, e.g., reduce, the expression, of the viral genome in a cell or tissue of the hematopoietic lineage, including for example immune cells (e.g., antigen presenting cells or APC, including dendritic cells (DCs), macrophages, and B-lymphocytes). In some embodiments, a miRNA, e.g., a miR-1, may be encoded in the viral genome to modulate, e.g., reduce, the expression, of the viral genome in a cell or tissue of the heart.

### Viral Genome Component: miR Binding Site

Tissue- or cell-specific expression of the AAV viral particles of the invention can be enhanced by introducing tissue- or cell-specific regulatory sequences, e.g., promoters, enhancers, microRNA binding sites, e.g., a detargeting site. Without wishing to be bound by theory, it is believed that an encoded miR binding site can modulate, e.g., prevent, suppress, or otherwise inhibit, the expression of a gene of interest on the viral genome of the invention, based on the expression of the corresponding endogenous microRNA (miRNA) or a corresponding controlled exogenous miRNA in a tissue or cell, e.g., a non-targeting cell or tissue. In some embodiments, a miR binding site modulates, e.g., reduces, expression of the payload encoded by a viral genome of an AAV particle described herein in a cell or tissue where the corresponding mRNA is expressed. In some embodiments, the miR binding site modulates, e.g., reduces, expression of the encoded siRNA for SOD1 in a cell or tissue of the DRG, liver, hematopoietic lineage, or a combination thereof.

In some embodiments, the viral genome of an AAV particle described herein comprises a nucleotide sequence encoding a microRNA binding site, e.g., a detargeting site. In some embodiments, the viral genome of an AAV particle described herein comprises a nucleotide sequence encoding a miR binding site, a microRNA binding site series (miR BSs), or a reverse complement thereof.

In some embodiments, the nucleotide sequence encoding the miR binding site series or the miR binding site is located in the 3′-UTR region of the viral genome (e.g., 3′ relative to the nucleic acid sequence encoding a payload), e.g., before the polyA sequence, 5′-UTR region of the viral genome (e.g., 5′ relative to the nucleic acid sequence encoding a payload), or both.

In some embodiments, the encoded miR binding site series comprise at least 1-5 copies, e.g., at least 1-3, 2-4, 3-5, 1, 2, 3, 4, 5 or more copies of a miR binding site (miR BS). In some embodiments, the encoded miR binding site series comprises 4 copies of a miR binding site. In some embodiments, all copies are identical, e.g., comprise the same miR binding site. In some embodiments, the miR binding sites within the encoded miR binding site series are continuous and not separated by a spacer. In some embodiments, the miR binding sites within an encoded miR binding site series are separated by a spacer, e.g., a non-coding sequence. In some embodiments, the spacer is about 1 to 6 nucleotides or about 5 to 10 nucleotides, e.g., about 7-8 nucleotides, nucleotides in length. In some embodiments, the spacer is about 8 nucleotides in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence GATAGTTA, or a nucleotide sequence having at least one, two, or three modifications, e.g., substitutions, insertions, or deletions, but no more than four modifications, e.g., substitutions, insertions, or deletions, of GATAGTTA.

In some embodiments, the encoded miR binding site series comprise at least 1-5 copies, e.g., at least 1-3, 2-4, 3-5, 1, 2, 3, 4, 5 or more copies of a miR binding site (miR BS). In some embodiments, at least 1, 2, 3, 4, 5, or all of the copies are different, e.g., comprise a different miR binding site. In some embodiments, the miR binding sites within the encoded miR binding site series are continuous and not separated by a spacer. In some embodiments, the miR binding sites within an encoded miR binding site series are separated by a spacer, e.g., a non-coding sequence. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, e.g., substitutions, insertions, or deletions, but no more than four modifications, e.g., substitutions, insertions, or deletions, of GATAGTTA.

In some embodiments, the encoded miR binding site is substantially identical (e.g., at least 70%, 75%, 80%, 85%, 90%, 95%, 99% or 100% identical), to the miR in the host cell. In some embodiments, the encoded miR binding site comprises at least 1, 2, 3, 4, or 5 mismatches or no more than 6, 7, 8, 9, or 10 mismatches to a miR in the host cell. In some embodiments, the mismatched nucleotides are contiguous. In some embodiments, the mismatched nucleotides are non-contiguous. In some embodiments, the mismatched nucleotides occur outside the seed region-binding sequence of the miR binding site, such as at one or both ends of the miR binding site. In some embodiments, the encoded miR binding site is 100% identical to the miR in the host cell.

In some embodiments, the nucleotide sequence encoding the miR binding site is substantially complimentary (e.g., at least 70%, 75%, 80%, 85%, 90%, 95%, 99% or 100% complementary), to the miR in the host cell. In some embodiments, the sequence complementary to the nucleotide sequence encoding the miR binding site comprises at least 1, 2, 3, 4, or 5 mismatches or no more than 6, 7, 8, 9, or 10 mismatches relative to the corresponding miR in the host cell. In some embodiments, the mismatched nucleotides are contiguous. In some embodiments, the mismatched nucleotides are non-contiguous. In some embodiments, the mismatched nucleotides occur outside the seed region-binding sequence of the miR binding site, such as at one or both ends of the miR binding site. In some embodiments, the encoded miR binding site is 100% complementary to the miR in the host cell.

In some embodiments, the encoded miR binding site or the encoded miR binding site series is about 10 to about 125 nucleotides in length, e.g., about 10 to 50 nucleotides, 10 to 100 nucleotides, 50 to 100 nucleotides, 50 to 125 nucleotides, or 100 to 125 nucleotides in length. In some embodiments, an encoded miR binding site or the encoded miR binding site series is about 7 to about 28 nucleotides in length, e.g., about 8-28 nucleotides, 7-28 nucleotides, 8-18 nucleotides, 12-28 nucleotides, 20-26 nucleotides, 22 nucleotides, 24 nucleotides, or 26 nucleotides in length, and optionally comprises at least one consecutive region (e.g., 7 or 8 nucleotides) complementary (e.g., full complementary or partially complementary) to the seed sequence of a miRNA (e.g., a miR122, a miR142, a miR-1, a miR183).

In some embodiments, the encoded miR binding site is complementary (e.g., fully complementary or partially complementary) to a miR expressed in liver or hepatocytes, such as miR122. In some embodiments, the encoded miR binding site or encoded miR binding site series comprises a miR122 binding site sequence. In some embodiments, the encoded miR 122 binding site comprises the nucleotide sequence of ACAAACACCATTGTCACACTCCA (SEQ ID NO: 61), or a nucleotide sequence comprising at least 50%, 55%, 60%, 65%, 70%, 75%, 80%, 85%, 90%, at least 95%, at least 99%, or 100% sequence identity, or having at least one, two, three, four, five, six, or seven modifications, e.g., substitutions, insertions, or deletions, but no more than ten modifications, e.g., substitutions, insertions, or deletions, to SEQ ID NO: 61, e.g., wherein the modification can result in a mismatch between the encoded miR binding site and the corresponding miRNA. In some embodiments, the viral genome comprises at least 3, 4, or 5 copies of the encoded miR122 binding site, e.g., an encoded miR 122 binding site series, optionally wherein the encoded miR122 binding site series comprises the nucleotide sequence of: ACAAACACCATTGTCACACTCCACACAAACACCATTGTCACACTCCACACAAACACCATT GTCACACTCCA (SEQ ID NO: 62), or a nucleotide sequence comprising at least 50%, 55%, 60%, 65%, 70%, 75%, 80%, 85%, 90%, at least 95%, at least 99%, or 100% sequence identity, or having at least one, two, three, four, five, six, or seven modifications, e.g., substitutions, insertions, or deletions, but no more than ten modifications, e.g., substitutions, insertions, or deletions, to SEQ ID NO: 62, e.g., wherein the modification can result in a mismatch between the encoded miR binding site and the corresponding miRNA. In some embodiments, at least two of the encoded miR122 binding sites are connected directly, e.g., without a spacer. In other embodiments, at least two of the encoded miR122 binding sites are separated by a spacer, e.g., 1, 2, 3, 4, 5, 6, 7, 8, 9, or 10 nucleotides in length, which is located between two or more consecutive encoded miR 122 binding site sequences. In embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence comprising at least one, two, or three modifications, e.g., substitutions, insertions, or deletions, but no more than four modifications, e.g., substitutions, insertions, or deletions, of GATAGTTA.

In some embodiments, the encoded miR binding site is complementary (e.g., fully or partially complementary) to a miR expressed in the heart. In embodiments, the encoded miR binding site or encoded miR binding site series comprises a miR-1 binding site. In some embodiments, the encoded miR-1 binding site comprises the nucleotide sequence of ATACATACTTCTTTACATTCCA (SEQ ID NO: 4679), a nucleotide sequence comprising at least 50%, 55%, 60%, 65%, 70%, 75%, 80%, 85%, 90%, at least 95%, at least 99%, or 100% sequence identity, or having at least one, two, three, four, five, six, or seven modifications e.g., substitutions, insertions, or deletions, but no more than ten modifications, e.g., substitutions, insertions, or deletions, to SEQ ID NO: 4679, e.g., wherein the modification can result in a mismatch between the encoded miR binding site and the corresponding miRNA. In some embodiments, the viral genome comprises at least 2, 3, 4, or 5 copies of the encoded miR-1 binding site, e.g., an encoded miR-1 binding site series. In some embodiments, the at least 2, 3, 4, or 5 copies (e.g., 2 or 3 copies) of the encoded miR-1 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, e.g., substitutions, insertions, or deletions, but no more than four modifications, e.g., substitutions, insertions, or deletions, of GATAGTTA.

In some embodiments, the encoded miR binding site is complementary (e.g., fully complementary or partially complementary) to a miR expressed in hematopoietic lineage, including immune cells (e.g., antigen presenting cells or APC, including dendritic cells (DCs), macrophages, and B-lymphocytes). In some embodiments, the encoded miR binding site is complementary (e.g., fully complementary or partially complementary) to a miR expressed in hematopoietic lineage comprises a nucleotide sequence disclosed, e.g., in US 2018/0066279, the contents of which are incorporated by reference herein in its entirety.

In some embodiments, the encoded miR binding site or encoded miR binding site series comprises a miR-142-3p binding site sequence. In some embodiments, the encoded miR-142-3p binding site comprises the nucleotide sequence of TCCATAAAGTAGGAAACACTACA (SEQ ID NO: 65), a nucleotide sequence comprising at least 50%, 55%, 60%, 65%, 70%, 75%, 80%, 85%, 90%, at least 95%, at least 99%, or 100% sequence identity, or having at least one, two, three, four, five, six, or seven modifications e.g., substitutions, insertions, or deletions, but no more than ten modifications, e.g., substitutions, insertions, or deletions, to SEQ ID NO: 65, e.g., wherein the modification can result in a mismatch between the encoded miR binding site and the corresponding miRNA. In some embodiments, the viral genome comprises at least 3, 4, or 5 copies of an encoded miR-142-3p binding site, e.g., an encoded miR-142-3p binding site series. In some embodiments, the at least 3, 4, or 5 copies (e.g., 4 copies) of the encoded miR-142-3p binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, e.g., substitutions, insertions, or deletions, but no more than four modifications, e.g., substitutions, insertions, or deletions, of GATAGTTA.

In some embodiments, the encoded miR binding site is complementary (e.g., fully complementary or partially complementary) to a miR expressed in a DRG (dorsal root ganglion) neuron, e.g., a miR183, a miR182, and/or miR96 binding site. In some embodiments, the encoded miR binding site is complementary (e.g., fully complementary or partially complementary) to a miR expressed in expressed in a DRG neuron. In some embodiments, the encoded miR binding site comprises a nucleotide sequence disclosed, e.g., in WO2020/132455, the contents of which are incorporated by reference herein in its entirety.

In some embodiments, the encoded miR binding site or encoded miR binding site series comprises a miR183 binding site sequence. In some embodiments, the encoded miR 183 binding site comprises the nucleotide sequence of AGTGAATTCTACCAGTGCCATA (SEQ ID NO: 60), or a nucleotide sequence comprising at least 50%, 55%, 60%, 65%, 70%, 75%, 80%, 85%, 90%, at least 95%, at least 99%, or 100% sequence identity, or having at least one, two, three, four, five, six, or seven modifications, e.g., substitutions, insertions, or deletions, but no more than ten modifications, e.g., substitutions, insertions, or deletions, to SEQ ID NO: 60, e.g., wherein the modification can result in a mismatch between the encoded miR binding site and the corresponding miRNA. In some embodiments, the sequence complementary (e.g., fully complementary or partially complementary) to the seed sequence corresponds to the double underlined of the encoded miR-183 binding site sequence. In some embodiments, the viral genome comprises at least comprises at least 3, 4, or 5 copies (e.g., 4 copies) of the encoded miR183 binding site, e.g. an encoded miR183 binding site. In some embodiments, the viral genome comprises at least comprises 4 copies of the encoded miR183 binding site, e.g. an encoded miR183 binding site comprising 4 copies of a miR183 binding site. In some embodiments, the at least 3, 4, or 5 copies (e.g., 4 copies) of the encoded miR183 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA, or a nucleotide sequence having at least one, two, or three modifications, e.g., substitutions, insertions, or deletions, but no more than four modifications, e.g., substitutions, insertions, or deletions, of GATAGTTA. In some embodiments, the spacer sequence comprises one or more of (i) GGAT; (ii) CACGTG; (iii) GCATGC, or a repeat of one or more of (i)-(iii).

In some embodiments, the encoded miR binding site or encoded miR binding site series comprises a miR182 binding site sequence. In some embodiments, the encoded miR 182 binding site comprises, the nucleotide sequence of AGTGTGAGTTCTACCATTGCCAAA (SEQ ID NO: 63), a nucleotide sequence comprising at least 50%, 55%, 60%, 65%, 70%, 75%, 80%, 85%, 90%, at least 95%, at least 99%, or 100% sequence identity, or having at least one, two, three, four, five, six, or seven modifications, e.g., substitutions, insertions, or deletions, but no more than ten modifications, e.g., substitutions, insertions, or deletions, to SEQ ID NO: 63, e.g., wherein the modification can result in a mismatch between the encoded miR binding site and the corresponding miRNA. In some embodiments, the viral genome comprises at least 2, 3, 4, or 5 copies of the encoded miR182 binding site, e.g., an encoded miR182 binding site series. In some embodiments, the at least 2, 3, 4, or 5 copies (e.g., 2 or 3 copies) of the encoded miR182 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA, or a nucleotide sequence having at least one, two, or three modifications, e.g., substitutions, insertions, or deletions, but no more than four modifications, e.g., substitutions, insertions, or deletions, of GATAGTTA. In some embodiments, the spacer sequence comprises one or more of (i) GGAT; (ii) CACGTG; (iii) GCATGC, or a repeat of one or more of (i)-(iii).

In some embodiments, the encoded miR binding site or encoded miR binding site series comprises a miR96 binding site sequence. In some embodiments, the encoded miR96 binding site comprises the nucleotide sequence of AGCAAAAATGTGCTAGTGCCAAA (SEQ ID NO: 64), a sequence comprising at least 50%, 55%, 60%, 65%, 70%, 75%, 80%, 85%, 90%, at least 95%, at least 99%, or 100% sequence identity, or having at least one, two, three, four, five, six, or seven modifications, e.g., substitutions, insertions, or deletions, but no more than ten modifications, e.g., substitutions, insertions, or deletions, to SEQ ID NO: 64, e.g., wherein the modification can result in a mismatch between the encoded miR binding site and the corresponding miRNA. In some embodiments, the viral genome comprises at least 2, 3, 4, or 5 copies of the encoded miR96 binding site, e.g., an encoded miR96 binding site series. In some embodiments, the at least 2, 3, 4, or 5 copies (e.g., 2 or 3 copies) of the encoded miR96 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA, or a nucleotide sequence having at least one, two, or three modifications, e.g., substitutions, insertions, or deletions, but no more than four modifications, e.g., substitutions, insertions, or deletions, of GATAGTTA. In some embodiments, the spacer sequence comprises one or more of (i) GGAT; (ii) CACGTG; (iii) GCATGC, or a repeat of one or more of (i)-(iii).

In some embodiments, the encoded miR binding site series comprises a miR122 binding site, a miR-1, a miR142 binding site, a miR183 binding site, a miR182 binding site, a miR 96 binding site, or a combination thereof. In some embodiments, the encoded miR binding site series comprises at least 2, 3, 4, or 5 copies of a miR122 binding site, a miR142 binding site, a miR183 binding site, a miR182 binding site, a miR 96 binding site, or a combination thereof. In some embodiments, at least two of the encoded miR binding sites are connected directly, e.g., without a spacer. In other embodiments, at least two of the encoded miR binding sites are separated by a spacer, e.g., 1, 2, 3, 4, 5, 6, 7, 8, 9, or 10 nucleotides in length, which is located between two or more consecutive encoded miR binding site sequences. In embodiments, the spacer is at least about 5 to 10 nucleotides, e.g., about 7-8 nucleotides or about 8 nucleotides, in length. In some embodiments, the spacer coding sequence or reverse complement thereof comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, e.g., substitutions, insertions, or deletions, but no more than four modifications, e.g., substitutions, insertions, or deletions, of GATAGTTA.

In some embodiments, an encoded miR binding site series comprises at least 2-5 copies (e.g., 2 or 3 copies) of a combination of at least two, three, four, five, or all of a miR-1, miR122 binding site, a miR142 binding site, a miR183 binding site, a miR182 binding site, a miR96 binding site, wherein each of the miR binding sites within the series are continuous (e.g., not separated by a spacer) or are separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, e.g., substitutions, insertions, or deletions, but no more than four modifications, e.g., substitutions, insertions, or deletions, of GATAGTTA.

In some embodiments, an encoded miR binding site series comprises at least 2-5 copies (e.g., 2 or 3 copies) of a combination of a miR-122 binding site and a miR-1 binding site, wherein each of the miR binding sites within the series are continuous (e.g., not separated by a spacer) or are separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, e.g., substitutions, insertions, or deletions, but no more than four modifications, e.g., substitutions, insertions, or deletions, of GATAGTTA.

### Payloads

In some embodiments, an AAV particle, e.g., an AAV particle for the vectorized delivery of an RNA agent, e.g., a siRNA, e.g., a SOD1 targeting siRNA described herein, comprises a payload. In some embodiments, an AAV particle, e.g., an AAV particle for the vectorized delivery of a siRNA described herein (e.g., a SOD1 targeting siRNA), comprises a viral genome encoding a payload. In some embodiments, the viral genome comprises a promoter operably linked to a nucleic acid encoding a payload. In some embodiments, the payload comprises a siRNA.

In some embodiments, the disclosure herein provides constructs that allow for improved expression of a SOD1 targeting siRNA delivered by gene therapy vectors.

In some embodiments, the disclosure provides constructs that allow for improved biodistribution of a SOD1 targeting siRNA delivered by gene therapy vectors.

In some embodiments, the disclosure provides constructs that allow for improved sub-cellular distribution or trafficking of a SOD1 targeting siRNA delivered by gene therapy vectors.

In some embodiments, the disclosure provides constructs that allow for improved trafficking of a SOD1 targeting siRNA to cells in the central nervous system, e.g., the brain and the spinal cord, delivered by gene therapy vectors.

In some aspects, the present disclosure relates to a composition containing or comprising a nucleic acid sequence encoding a SOD1 targeting siRNA or functional fragment or variants thereof and methods of administering the composition in vitro or in vivo in a subject, e.g., a humans and/or an animal model of disease, e.g., a disease related to expression of SOD1.

AAV particles of the present disclosure may comprise a nucleic acid sequence encoding at least one payload. In some embodiments, a payload or payload region comprises a polynucleotide or polynucleotide region encoded by or within a viral genome or an expression product of such polynucleotide or polynucleotide region, e.g., a polynucleotide encoding a SOD1 targeting polynucleotide (e.g., a SOD1 targeting RNA agent, e.g., a siRNA, e.g., a siRNA for inhibiting expression of SOD1) or fragment or variant thereof. The payload may comprise any nucleic acid known in the art that is useful for the expression (by supplementation of the SOD1 siRNA or gene replacement using a modulatory nucleic acid) of the SOD1 targeting siRNA in a target cell transduced or contacted with the AAV particle carrying the payload.

The payload construct may comprise a combination of coding and non-coding nucleic acid sequences.

Any segment, fragment, or the entirety of the viral genome and therein, the payload region, may be codon optimized.

In some embodiments, the viral genome encodes more than one payload. As a non-limiting example, a viral genome encoding more than one payload may be replicated and packaged into a viral particle. A target cell transduced with a viral particle comprising more than one payload may express each of the payloads in a single cell.

In some embodiments, the viral genome may encode a coding or non-coding RNA. In certain embodiments, the adeno-associated viral vector particle further comprises at least one cis-element selected from the group consisting of a Kozak sequence, a backbone sequence, and an intron sequence.

In some embodiments, the payload is a polypeptide which may be a peptide or protein. A protein encoded by the payload construct may comprise a secreted protein, an intracellular protein, an extracellular protein, and/or a membrane protein. The encoded proteins may be structural or functional. Proteins encoded by the viral genome include, but are not limited to, mammalian proteins. In certain embodiments, the AAV particle contains a viral genome that encodes a SOD1 targeting polynucleotide (e.g., a SOD1 targeting RNAi, e.g., a siRNA for inhibiting expression of SOD1), or a fragment or variant thereof. The AAV particles described herein may be useful in the fields of human disease, veterinary applications, and a variety of in vivo and in vitro settings.

In some embodiments, a payload may comprise polypeptides that serve as marker proteins to assess cell transformation and expression, fusion proteins, polypeptides having a desired biological activity, gene products that can complement a genetic defect, RNA molecules, transcription factors, and other gene products that are of interest in regulation and/or expression. In some embodiments, a payload may comprise nucleotide sequences that provide a desired effect or regulatory function (e.g., transposons, transcription factors).

The encoded payload may comprise a gene therapy product. A gene therapy product may include, but is not limited to, a polypeptide, RNA molecule, or other gene product that, when expressed in a target cell, provides a desired therapeutic effect. In some embodiments, a gene therapy product may comprise a substitute for a non-functional gene or a gene that is absent, expressed in insufficient amounts, or mutated. In some embodiments, a gene therapy product may comprise a substitute for a non-functional protein or polypeptide or a protein or polypeptide that is absent, expressed in insufficient amounts, misfolded, degraded too rapidly, or mutated. For example, a gene therapy product may comprise a SOD1 siRNA or a polynucleotide encoding a SOD1 siRNA to treat SOD1-related disorders.

In some embodiments, the payload encodes a messenger RNA (mRNA). As used herein, the term “messenger RNA” (mRNA) refers to any polynucleotide that encodes a polypeptide of interest and that is capable of being translated to produce the encoded polypeptide of interest in vitro, in vivo, in situ, or ex vivo.

The components of an mRNA include, but are not limited to, a coding region, a 5′-UTR (untranslated region), a 3′-UTR, a 5′-cap and a poly-A tail. In some embodiments, the encoded mRNA or any portion of the AAV genome may be codon optimized.

A payload construct encoding a payload may comprise or encode a selectable marker. A selectable marker may comprise a gene sequence or a protein or polypeptide encoded by a gene sequence expressed in a host cell that allows for the identification, selection, and/or purification of the host cell from a population of cells that may or may not express the selectable marker. In some embodiments, the selectable marker provides resistance to survive a selection process that would otherwise kill the host cell, such as treatment with an antibiotic. In some embodiments, an antibiotic selectable marker may comprise one or more antibiotic resistance factors, including but not limited to neomycin resistance (e.g., neo), hygromycin resistance, kanamycin resistance, and/or puromycin resistance.

In some embodiments, a payload construct encoding a payload may comprise a selectable marker including, but not limited to, β-lactamase, luciferase, β-galactosidase, or any other reporter gene as that term is understood in the art, including cell-surface markers, such as CD4 or the truncated nerve growth factor (NGFR) (for GFP, see WO 96/23810; Heim et al., Current Biology 2:178-182 (1996); Heim et al., Proc. Natl. Acad. Sci. USA (1995); or Heim et al., Science 373:663-664 (1995); for β-lactamase, see WO 96/30540); the contents of each of which are herein incorporated by reference in their entirety.

In some embodiments, a payload construct encoding a selectable marker may comprise a fluorescent protein. A fluorescent protein as herein described may comprise any fluorescent marker including but not limited to green, yellow, and/or red fluorescent protein (GFP, YFP, and/or RFP). In some embodiments, a payload construct encoding a selectable marker may comprise a human influenza hemagglutinin (HA) tag.

In certain embodiments, a nucleic acid for expression of a payload in a target cell will be incorporated into the viral genome and located between two ITR sequences.

In some embodiments, a payload construct further comprises a nucleic acid sequence encoding a peptide that binds to the cation-independent mannose 6-phosphate (M6P) receptor (CI-MPR) with high affinity, as described in Int'l Pat. App. Pub. No. WO2019213180A1, the disclosure of which is incorporated herein by reference in its entirety. The peptide that binds CI-MPR can be, e.g., an IGF2 peptide or variant thereof. Binding of CI-MPR can facilitate cellular uptake or delivery and intracellular or sub-cellular targeting of therapeutic proteins provided by gene therapy vectors.

The payload region may be constructed in such a way as to reflect a region similar to or mirroring the natural organization of an mRNA, e.g., the mRNA encoded by SOD1.

### Modulatory Polynucleotides

In some embodiments, modulatory polynucleotides, e.g., RNA or DNA molecules, may be used to treat neurodegenerative disease, e.g., amyotrophic lateral sclerosis (ALS).

In some embodiments, the modulatory polynucleotide comprises a nucleotide sequence encoding a pri-miRNA, a pre-miRNA, miRNA, or a siRNA. In some embodiments, the pri-miRNA or pre-miRNA is cleaved by cellular enzymes (e.g., Dicer) to produce a miRNA or siRNA. In some embodiments, the modulatory polynucleotide comprises a nucleotide sequence encoding a siRNA within a pri-miRNA or pre-miRNA scaffold sequence. In such cases, cleavage of the pre-miRNA or pri-miRNA by cellular enzymes (e.g., Dicer) produces a siRNA.

In some embodiments, the modulatory polynucleotides may comprise at least one nucleic acid sequence encoding at least one siRNA molecule. The nucleic acids may, independently if there is more than one, encode 1, 2, 3, 4, 5, 6, 7, 8, 9, or more than 9 siRNA molecules.

The present disclosure relates, in part, to RNA interfering (RNAi) induced inhibition of gene expression for treating neurodegenerative disorders. Provided are siRNAs or dsRNA that target SOD1 gene. Such siRNAs or dsRNA can silence SOD1 gene expression in cells, for example, motor neurons, therefore, ameliorating symptoms of ALS such as motor death and muscle atrophy. The SOD1 siRNA may be encoded in polynucleotides of a recombinant AAV genome.

siRNA duplexes or dsRNA targeting a specific mRNA may be designed and synthesized as part of a target SOD1 targeting polynucleotide in vitro and introduced into cells for activating RNAi process.

siRNA Molecules

The present disclosure relates to RNA interference (RNAi) induced inhibition of gene expression for treating neurodegenerative disorders. Provided herein are RNA agents, e.g., siRNA duplexes or encoded dsRNA that target the gene of interest. Such siRNA duplexes or encoded dsRNA can reduce or silence gene expression in cells, such as, but not limited to, medium spiny neurons, cortical neurons and/or astrocytes.

RNAi (also known as post-transcriptional gene silencing (PTGS), quelling, or co-suppression) is a post-transcriptional gene silencing process in which RNA molecules, in a sequence-specific manner, inhibit gene expression, typically by causing the destruction of specific mRNA molecules. The active components of RNAi are short/small double stranded RNAs (dsRNAs), called small interfering RNAs (siRNAs), that typically contain 15-30 nucleotides (e.g., 19 to 25, 19 to 24 or 19-21 nucleotides) and 2 nucleotide 3′ overhangs, and that match the nucleic acid sequence of the target gene. These short RNA species may be naturally produced in vivo by Dicer-mediated cleavage of larger dsRNAs and they are functional in mammalian cells.

Naturally expressed small RNA molecules, named microRNAs (miRNAs), elicit gene silencing by regulating the expression of mRNAs. The miRNAs-containing RNA Induced Silencing Complex (RISC) targets mRNAs presenting a perfect sequence complementarity with nucleotides 2-7 in the 5′ region of the miRNA which is called the seed region, and other base pairs with its 3′ region. miRNA-mediated downregulation of gene expression may be caused by cleavage of the target mRNAs, translational inhibition of the target mRNAs, or mRNA decay. miRNA targeting sequences are usually located in the 3′-UTR of the target mRNAs. A single miRNA may target more than 100 transcripts from various genes, and one mRNA may be targeted by different miRNAs.

siRNA duplexes or dsRNA targeting a specific mRNA may be designed and synthesized in vitro and introduced into cells for activating RNAi processes (e.g., as described in Elbashir S M et al., Nature, 2001, 411, 494-498, the contents of which are hereby incorporated by reference in their entirety).

In vitro synthetized siRNA molecules may be introduced into cells to activate RNAi. An exogenous siRNA duplex, when it is introduced into cells, similar to the endogenous dsRNAs, can be assembled to form the RNA Induced Silencing Complex (RISC), a multiunit complex that interacts with RNA sequences that are complementary to one of the two strands of the siRNA duplex (i.e., the antisense strand). During the process, the sense strand (or passenger strand) of the siRNA is lost from the complex, while the antisense strand (or guide strand) of the siRNA is matched with its complementary RNA. In particular, the targets of siRNA containing RISC complexes are mRNAs presenting a perfect sequence complementarity. Then, siRNA mediated gene silencing occurs by cleaving, releasing and degrading the target.

The siRNA duplex comprised of a sense strand homologous to the target RNA transcript and an antisense strand that is complementary to the target RNA transcript offers much more advantage in terms of efficiency for target RNA destruction compared to the use of the single strand (ss)-siRNAs (e.g., antisense strand RNA or antisense oligonucleotides). In many cases, it requires higher concentration of the ss-siRNA to achieve the effective gene silencing potency of the corresponding duplex.

While not wishing to be bound by theory, a vector genome may encode a pri-miRNA or pre-miRNA, which when expressed in the target cells is processed via the cell's endogenous pathway to a miRNA duplex. Once the miRNA duplex unwinds, the guide strand may bind to its complementary mRNA substrate and recruit the RNA-induced silencing complex, which leads to the cleavage of the substrate mRNA. Hence, delivery of an AAV particle comprising a vector genome encoding an RNAi precursor (pre- or pri-miR), enters the processing pathway at a different step than would occur with direct administration of an shRNA or siRNA, for example.

Any of the foregoing molecules may be encoded by an AAV viral genome. In some embodiments, a nucleic acid sequence encoding such siRNA molecules, or a single strand of the siRNA molecules, is inserted into the vector genome of an AAV particle and introduced into cells, specifically neurons, including interneurons, and/or other surrounding cells in the central nervous system.

In some embodiments, the SOD1 targeting polynucleotide is a single-stranded antisense RNAi molecule, a single-stranded siRNA, or a double-stranded RNAi (e.g., a siRNA duplex) that inhibits expression of SOD1. In some embodiments, the SOD1 targeting polynucleotide is a siRNA duplex comprising a sense strand and an antisense strand.

The present disclosure provides a small interfering RNA (siRNA) for targetting SOD1 and AAV viral genomes AAV particles, and/or modulatory polynucleotides encoding said siRNA.

In some embodiments, the siRNA can comprise an overhang of at least one nucleotide; alternatively the overhang can comprise at least two nucleotides, at least three nucleotides, at least four nucleotides, at least five nucleotides or more. In some embodiments, the overhang is or comprises a nucleotide/nucleoside analog, including a deoxynucleotide/nucleoside. The overhang(s) may be on the sense strand, the antisense strand or any combination thereof. Furthermore, the nucleotide(s) of an overhang can be present on the 5′ end, 3′ end or both ends of either an antisense or sense strand of a siRNA.

The encoded siRNA of the present disclosure contains an antisense strand and a sense strand hybridized together forming a duplex structure. In some embodiments, the antisense strand is complementary to the nucleic acid sequence of the targeted SOD1 gene, and the sense strand is homologous to the nucleic acid sequence of the targeted SOD1 gene.

In some embodiments, the SOD1 targeting polynucleotide is a siRNA comprising a sense strand and an antisense strand.

In some embodiments, an siRNA molecule of the present disclosure comprises a sense strand and a corresponding antisense strand. In some embodiments, the antisense strand is complementary, e.g., substantially complementary or fully complementary to at least a portion of a SOD1 gene, e.g., a SOD1 gene as described in Table 19, or a variant thereof (e.g., a human SOD1). In some embodiments, the antisense strand is complementary, e.g., partially complementary or fully complementary, to a region of the SOD1 transcript, as described in Table 19.

In some embodiments, the antisense strand sequence comprises a region of complementarity to the target sequence (e.g., a SOD1 target), which is 15-30, 19-21, or 25-30 nucleotides in length, e.g., 15, 16, 17, 18, 19, 20, 21, 22, 23, 24, 25, 26, 27, 28, 29, or 30 nucleotides in length. In some embodiments, the antisense strand sequence is fully complementary to the target sequence (e.g., 100% complementary). In some embodiments, the antisense strand sequence is substantially complementary to the target sequence. In some embodiments, the antisense strand sequence comprises a region of complementarity to a portion of a the target sequence (e.g., a SOD1 sequence provided in Table 19 or a variant thereof), e.g., a portion of at least 15, 16, 17, 18, 19, 20, or 21 contiguous nucleotides that comprises one, two, or three mismatches relative to the target sequence.

In some embodiments, the sense strand sequence comprises at least 15, 16, 17, 18, 19, 20, or 21 contiguous nucleotides with 0, 1, 2, or 3 mismatches of the corresponding portion of a SOD1 sequence, e.g., a SOD1 sequence provided in Table 19, or a variant thereof.

In some embodiments, an siRNA comprises a region, e.g., a region that is 30 nucleotides or less, generally 19-24, nucleotides in length, that is substantially complementary (e.g., at least 70%, 75%, 80%, 85%, 90%, 95%, 99% or 100% complementary) to at least part of an mRNA transcript of the SOD1 gene (e.g., a human variant of the SOD1 gene). In some embodiments, the antisense sequence comprises or consists of a nucleotide sequence that is fully complementary or substantially complementary (e.g., at least 70%, 75%, 80%, 85%, 90%, 95%, 99% or 100% complementary) to the nucleotides of the SOD1 transcript.

In embodiments, SOD1 gene targeted by an siRNA described herein comprises a wild type SOD1 gene, mRNA, and/or protein (e.g., a SOD1 sequence provided in Table 19); a mutated SOD1 gene, mRNA, and/or protein comprising at least one mutation; or a combination thereof (e.g., a mutation as described in Table 1).

In some embodiments, the region that is substantially complementary may be 30 nucleotides or less (e.g., 29, 28, 27, 26, 25, 24, 23, 22, 21, 20, 19, 18, 17, 16, or 15 nucleotides in length). In some embodiments, the region of complementarity may range from 15, 16, 17, 18, 19, 20, 21, 22, 23, 24, 25, 26, 27, 28, or 29 nucleotides up to 16, 17, 18, 19, 20, 21, 22, 23, 24, 25, 26, 27, 28, 29, or 30 nucleotides in length.

In some embodiments, an siRNA molecule of the present disclosure comprises a sense strand and a complementary antisense strand in which both strands are hybridized together to form a duplex structure. The antisense strand has sufficient complementarity to the SOD1 mRNA sequence to direct target-specific RNAi, e.g., , the siRNA molecule has a sequence sufficient to trigger the destruction of the target mRNA by the RNAi machinery or process.

In certain embodiments, an siRNA molecule of the present disclosure comprises a sense strand and a complementary antisense strand in which both strands are hybridized together to form a duplex structure and where the start site of the hybridization to the SOD1 mRNA is between nucleotide 15 and 1000 on the SOD1 mRNA sequence. As a non-limiting example, the start site may be between nucleotide 15-25, 15-50, 15-75, 15-100, 100-150, 150-200, 200-250, 250-300, 300-350, 350-400, 400-450, 450-500, 500-550, 550-600, 600-650, 650-700, 700-70, 750-800, 800-850, 850-900, 900-950, and 950-1000 on the SOD1 mRNA sequence.

In some embodiments, the antisense strand and target SOD1 mRNA sequences are fully complementary, e.g., are 100% complementary. The antisense strand may be complementary to any part of the target SOD1 mRNA sequence (e.g., a SOD1 sequence of Table 19). Alternatively, the antisense strand may have 0, 1, 2, or 3 mismatches or no more than 4, 5, or 6 mismatches to the target SOD1 mRNA sequence (e.g., a SOD1 sequence of Table 19). In some embodiments, the mismatched nucleotides are contiguous. In some embodiments, the mismatched nucleotides are non-contiguous. In some embodiments, the mismatched nucleotides occur outside the SOD1 target mRNA binding site, such as at one or both ends of the antisense strand. As a non-limiting example, the antisense strand and the SOD1 mRNA sequence (e.g., a SOD1 sequence of Table 19) have at least 80%, 81%, 82%, 83%, 84%, 85%, 86%, 87%, 88%, 89%, 90%, 91%, 92%, 93%, 94%, 95%, 96%, 97%, 98% or 99% or at least 80-90%, 80-95%, 80-99%, 90-95%, 90-99% or 95-99% complementarity.

In some embodiments, the sense strand sequence and the antisense strand sequence of an siRNA described herein comprise a region of complementarity, which is 15-30, 19-21, or 25-30 nucleotides in length, e.g., 17, 18, 20, 21, 22, 25, or 30 nucleotides in length. In some embodiments, the sense strand, antisense strand, or both comprise at least 15-30, 19-21, or 25-30 nucleotides in length, e.g., 17, 18, 20, 21, 22, 25, or 30 nucleotides in length. In some embodiments, the sense strand, antisense strand, or both the sense strand comprise one or two nucleotide overhangs, e.g., at the 5′ end of the sense strand and the 3′ end of the antisense strand or at the 5′ end of the antisense strand and the 3′ end of the sense strand. In some embodiments, the sense strand and the antisense strand comprise one mismatch. In some embodiments, the antisense strand sequence comprises one mismatch with the target sequence. In some embodiments, the sense strand and the antisense strand are fully complementary.

According to the present disclosure, the siRNA molecule targeting SOD1 has a length from about 10-50 or more nucleotides, e.g., each strand comprising 10-50 nucleotides (or nucleotide analogs). In some embodiments, the siRNA molecule has a length from about 15-30, e.g., 15, 16, 17, 18, 19, 20, 21, 22, 23, 24, 25, 26, 27, 28, 29, or 30 nucleotides in each strand. In certain embodiments, each strand of the siRNA molecule has a length from about 19 to 25, 19 to 24 or 19 to 21 nucleotides. In some embodiments, each strand of the siRNA molecule has a length ranging from 15, 16, 17, 18, 19, 20, 21, 22, 23, 24, 25, 26, 27, 28, 29, or 30 nucleotides up to 16, 17, 18, 19, 20, 21, 22, 23, 24, 25, 26, 27, 28, 29, 30 or 31 nucleotides. In some embodiments, at least one strand of the siRNA molecule (e.g., the sense strand and/or the antisense strand) is 19, 20, 21, 22, 23, 24, or 25 nucleotides in length. In some embodiments, the siRNA molecule comprises an antisense (e.g., guide) strand

sequence. In some embodiments, the antisense strand sequence comprises at least 15, 16, 17, 18, 19, 20, 21 contiguous nucleotides differing by 3, 2, 1, or 0 nucleotides from any one of the antisense strand sequences provided in Table 10. In some embodiments, the antisense sequence comprises the nucleotide sequence of any one of the antisense strand sequences provided in Table 10 or 14, or a nucleotide sequence 70%, 80%, 85%, 90%, 95% identical thereto. In some embodiments, the antisense strand sequence comprises at least 15, 16, 17, 18, 19, or more than 19 consecutive nucleotides of a nucleotide sequence of any one of the antisense strand sequences provided in Table 10 or 14. In some embodiments, the antisense sequence comprises nucleotides 1 to 19, 1 to 18, 1 to 17, 1 to 16, 2 to 19, or 2 to 18, 2 to 17 of any one of the antisense strand sequences provided in Table 10 or 14. In some embodiments, the antisense sequence may comprise a dTdT overhang, e.g., at the 3′ end of the antisense strand sequence. In some embodiments, the antisense sequence does not comprise a dTdT overhang, e.g., does not comprise a dTdT overhang at the 3′ end of the sequence. In some embodiments, the antisense strand sequence is a DNA sequence (e.g., comprises T in place of U in any one of the sequences provided in Tables 10). In some embodiments, the antisense strand sequence is an RNA sequence (e.g., comprises U in place of T in any one of the sequences provided in Table 14).

In some embodiments, the siRNA molecule comprises a sense (e.g., passenger) strand sequence. In some embodiments, the sense strand sequence comprises at least 15, 16, 17, 18, 19, 20, 21 contiguous nucleotides differing by 3, 2, 1, or 0 nucleotides from any one of the sense strand sequences provided in Table 10 or 14. In some embodiments, the sense sequence comprises the nucleotide sequence of any one of the sense strand sequences provided in Table 10 or 14, or a nucleotide sequence 70%, 80%, 85%, 90%, 95% identical thereto. In some embodiments, the sense strand sequence comprises at least 15, 16, 17, 18, 19, or more than 19 consecutive nucleotides of a nucleotide sequence of any one of the sense strand sequences provided in Table 10 or 14. In some embodiments, the sense sequence comprises nucleotides 1 to 19, 1 to 18, 1 to 17, 1 to 162 to 19, or 2 to 18, 2 to 17 of any one of the sense strand sequences provided in Table 10 or 14. In some embodiments, the sense sequence may comprise a dTdT overhang, e.g. at the 3′ end of the sense strand sequence. In some embodiments, the sense sequence does not comprise a dTdT overhang, e.g., does not comprise the dTdT overhang at the 3′ end of the sequence. In some embodiments, the sense strand sequence is a DNA sequence (e.g., comprises T in place of U in any one of the sequences provided in Table 10). In some embodiments, the sense strand sequence is an RNA sequence (e.g., comprises U in place of T in any one of the sequences provided in Table 14).

In some embodiments, the siRNA comprises an antisense strand sequence comprising a nucleotide sequence comprising at least 15, 16, 17, 18, 19, 20, or 21 contiguous nucleotides differing by no more than 3, 2, 1, or 0 nucleotides from the antisense strand sequences provided in Table 10 or 14; and a sense strand sequence comprising a nucleotide sequence comprising at least 15, 16, 17, 18, 19, 20, or 21 contiguous nucleotides differing by no more than 3, 2, 1, or 0 nucleotides from the sense strand sequences provided in Table 10 or 14, optionally wherein the sense strand sequence and the antisense strand sequence comprise a region of complementarity of at least 15 nucleotides. In some embodiments, the siRNA molecules of the present disclosure may comprise a siRNA duplex as described in Table 10 or 14, or a variant, e.g., functional variant, thereof.

It is understood that in some embodiments, any of the sequences in Table 10 herein, can comprise a U nucleotide (uridine) in place of any of or all T nucleotides (thymine) within the sequence (e.g., an RNA molecule). It is also understood that in some embodiments, that any sequence provided herein can comprise a T nucleotide (thymine) in place of any of or all U nucleotides (uracil) within the sequence (e.g., a DNA molecule).

In some embodiments, the siRNA molecules may be unmodified RNA molecules. In other aspects, the siRNA molecules may contain at least one modified nucleotide, such as base, sugar or backbone modifications. In some embodiments, the overhanging nucleotides at the 3′-end of each of the sense and the antisense strand is a dTdT overhang. In some embodiments, the sense strand, antisense strand, or both the sense strand and the antisense strand does not comprise a dTdT overhang.

TABLE 10
Sense and antisense strand sequences of SOD1 dsRNA

| | SIRNA | Sense | Sense Strand | | | Antisense Strand | AS |
|---|---|---|---|---|---|---|---|
| | Duplex | Strand | Sequence (5′-3′) | SS SEQ | AS | Sequence (5′-3′) | SEQ |
| Name | ID | ID | (Passenger) | ID | ID | (Guide) | ID |
| | D-2741 | 7414 | CGGAGGUCUGG | 84 | 7415 | UUUAUAGGCCA | 1052 |
| | | | CCUAUAACdTdT | | | GACCUCCGdTdT | |
| | D-2742 | 7416 | GGAGGUCUGGC | 85 | 7417 | UUUUAUAGGCC | 1053 |
| | | | CUAUAAACdTdT | | | AGACCUCCdTdT | |
| | D-2743 | 7418 | GAGGUCUGGCC | 86 | 7419 | UCUUUAUAGGC | 1054 |
| | | | UAUAAAGCdTdT | | | CAGACCUCdTdT | |
| | D-2744 | 7420 | AGGUCUGGCCU | 87 | 7421 | UACUUUAUAGG | 1055 |
| | | | AUAAAGUCdTdT | | | CCAGACCUdTdT | |
| | D-2745 | 7422 | GGUCUGGCCUA | 88 | 7423 | UUACUUUAUAG | 1056 |
| | | | UAAAGUACdTdT | | | GCCAGACCdTdT | |
| | D-2746 | 7424 | UCUGGCCUAUA | 89 | 7425 | UACUACUUUAU | 1057 |
| | | | AAGUAGUCdTdT | | | AGGCCAGAdTdT | |
| | D-2747 | 7426 | CUGGCCUAUAA | 90 | 7427 | UGACUACUUUA | 1058 |
| | | | AGUAGUCCdTdT | | | UAGGCCAGdTdT | |
| | D-2748 | 7428 | UGGCCUAUAAA | 91 | 7429 | UCGACUACUUU | 1059 |
| | | | GUAGUCGCdTdT | | | AUAGGCCAdTdT | |
| | D-2749 | 7430 | GGCCUAUAAAG | 92 | 7431 | UGCGACUACUU | 1060 |
| | | | UAGUCGCCdTdT | | | UAUAGGCCdTdT | |
| | D-2750 | 7432 | GCCUAUAAAGU | 93 | 7433 | UCGCGACUACU | 1061 |
| | | | AGUCGCGCdTdT | | | UUAUAGGCdTdT | |
| | D-2751 | 7434 | CCUAUAAAGUA | 94 | 7435 | UCCGCGACUAC | 1062 |
| | | | GUCGCGGCdTdT | | | UUUAUAGGdTdT | |
| | D-2752 | 7436 | GUCGUAGUCUC | 95 | 7437 | UGCUGCAGGAG | 1063 |
| | | | CUGCAGCCdTdT | | | ACUACGACdTdT | |
| | D-2753 | 7438 | CGUAGUCUCCU | 96 | 7439 | UACGCUGCAGG | 1064 |
| | | | GCAGCGUCdTdT | | | AGACUACGdTdT | |
| | D-2754 | 7440 | GUAGUCUCCUG | 97 | 7441 | UGACGCUGCAG | 1065 |
| | | | CAGCGUCCdTdT | | | GAGACUACdTdT | |
| | D-2755 | 7442 | UAGUCUCCUGC | 98 | 7443 | UAGACGCUGCA | 1066 |
| | | | AGCGUCUCdTdT | | | GGAGACUAdTdT | |
| | D-2756 | 7444 | AUGGCGACGAA | 99 | 7445 | UCACGGCCUUC | 1067 |
| | | | GGCCGUGCdTdT | | | GUCGCCAUdTdT | |
| | D-2757 | 7446 | CGACGAAGGCC | 100 | 7447 | UCGCACACGGCC | 1068 |
| | | | GUGUGCGCdTdT | | | UUCGUCGdTdT | |
| | D-2758 | 7448 | GAAGGCCGUGU | 101 | 7449 | UAGCACGCACA | 1069 |
| | | | GCGUGCUCdTdT | | | CGGCCUUCdTdT | |
| | D-2759 | 7450 | GGCCGUGUGCG | 102 | 7451 | UUUCAGCACGC | 1070 |
| | | | UGCUGAACdTdT | | | ACACGGCCdTdT | |
| | D-2760 | 7452 | AGGGCGACGGC | 103 | 7453 | UGCACUGGGCC | 1071 |
| | | | CCAGUGCCdTdT | | | GUCGCCCUdTdT | |
| | D-2761 | 7454 | UGCAGGGCAUC | 104 | 7455 | UAAUUGAUGAU | 1072 |
| | | | AUCAAUUCdTdT | | | GCCCUGCAdTdT | |
| | D-2762 | 7456 | GCAGGGCAUCA | 105 | 7457 | UAAAUUGAUGA | 1073 |
| | | | UCAAUUUCdTdT | | | UGCCCUGCdTdT | |
| | D-2763 | 7458 | AGGGCAUCAUC | 106 | 7459 | UCGAAAUUGAU | 1074 |
| | | | AAUUUCGCdTdT | | | GAUGCCCUdTdT | |
| | D-2764 | 7460 | GGGCAUCAUCA | 107 | 7461 | UUCGAAAUUGA | 1075 |
| | | | AUUUCGACdTdT | | | UGAUGCCCdTdT | |
| | D-2765 | 7462 | GGCAUCAUCAA | 108 | 7463 | UCUCGAAAUUG | 1076 |
| | | | UUUCGAGCdTdT | | | AUGAUGCCdTdT | |
| | D-2766 | 7464 | GCAUCAUCAAU | 113 | 7465 | UGCUCGAAAUU | 1077 |
| | | | UUCGAGCCdTdT | | | GAUGAUGCdTdT | |
| | D-2767 | 7466 | CAUCAUCAAUU | 114 | 7467 | UUGCUCGAAAU | 1078 |
| | | | UCGAGCACdTdT | | | UGAUGAUGdTdT | |
| | D-2768 | 7468 | AAUUUCGAGCA | 115 | 7469 | UUUCCUUCUGC | 1079 |
| | | | GAAGGAACdTdT | | | UCGAAAUUdTdT | |
| | D-2769 | 7470 | UUCGAGCAGAA | 116 | 7471 | UACUUUCCUUC | 1080 |
| | | | GGAAAGUCdTdT | | | UGCUCGAAdTdT | |
| | D-2770 | 7472 | UCGAGCAGAAG | 117 | 7473 | UUACUUUCCUU | 1081 |
| | | | GAAAGUACdTdT | | | CUGCUCGAdTdT | |
| | D-2771 | 7474 | AAGGUGUGGGG | 118 | 7475 | UAAUGCUUCCC | 1082 |
| | | | AAGCAUUCdTdT | | | CACACCUUdTdT | |
| | D-2772 | 7476 | GGUGUGGGGAA | 119 | 7477 | UUUAAUGCUUC | 1083 |
| | | | GCAUUAACdTdT | | | CCCACACCdTdT | |
| | D-2773 | 7478 | GACUGACUGAA | 120 | 7479 | UGCAGGCCUUC | 1084 |
| | | | GGCCUGCCdTdT | | | AGUCAGUCdTdT | |
| | D-2774 | 7480 | CUGACUGAAGG | 121 | 7481 | UAUGCAGGCCU | 1085 |
| | | | CCUGCAUCdTdT | | | UCAGUCAGdTdT | |
| | D-2775 | 7482 | UGACUGAAGGC | 122 | 7483 | UCAUGCAGGCC | 1086 |
| | | | CUGCAUGCdTdT | | | UUCAGUCAdTdT | |
| | D-2776 | 7484 | UGAAGGCCUGC | 123 | 7485 | UAAUCCAUGCA | 1087 |
| | | | AUGGAUUCdTdT | | | GGCCUUCAdTdT | |
| | D-2777 | 7486 | GAAGGCCUGCA | 124 | 7487 | UGAAUCCAUGC | 1088 |
| | | | UGGAUUCCdTdT | | | AGGCCUUCdTdT | |
| | D-2778 | 7488 | UGCAUGGAUUC | 131 | 7489 | UGAACAUGGAA | 1089 |
| | | | CAUGUUCCdTdT | | | UCCAUGCAdTdT | |
| | D-2779 | 7490 | CAUGGAUUCCA | 132 | 7491 | UAUGAACAUGG | 1090 |
| | | | UGUUCAUCdTdT | | | AAUCCAUGdTdT | |
| | D-2780 | 7492 | GGAUUCCAUGU | 133 | 7493 | UCUCAUGAACA | 1091 |
| | | | UCAUGAGCdTdT | | | UGGAAUCCdTdT | |
| | D-2781 | 7494 | UUCCAUGUUCA | 134 | 7495 | UAAACUCAUGA | 1092 |
| | | | UGAGUUUCdTdT | | | ACAUGGAAdTdT | |
| | D-2782 | 7496 | GUUCAUGAGUU | 135 | 7497 | UAUCUCCAAAC | 1093 |
| | | | UGGAGAUCdTdT | | | UCAUGAACdTdT | |
| | D-2783 | 7498 | UUCAUGAGUUU | 136 | 7499 | UUAUCUCCAAA | 1094 |
| | | | GGAGAUACdTdT | | | CUCAUGAAdTdT | |
| | D-2784 | 7500 | UGAGUUUGGAG | 139 | 7501 | UGUAUUAUCUC | 1095 |
| | | | AUAAUACCdTdT | | | CAAACUCAdTdT | |
| | D-2785 | 7502 | GAGUUUGGAGA | 140 | 7503 | UUGUAUUAUCU | 1096 |
| | | | UAAUACACdTdT | | | CCAAACUCdTdT | |
| | D-2786 | 7504 | AGGCUGUACCA | 141 | 7505 | UCCUGCACUGG | 1097 |
| | | | GUGCAGGCdTdT | | | UACAGCCUdTdT | |
| | D-2787 | 7506 | GGCUGUACCAG | 142 | 7507 | UACCUGCACUG | 1098 |
| | | | UGCAGGUCdTdT | | | GUACAGCCdTdT | |
| | D-2788 | 7508 | GCAGGUCCUCA | 143 | 7509 | UAUUAAAGUGA | 1099 |
| | | | CUUUAAUCdTdT | | | GGACCUGCdTdT | |
| | D-2789 | 7510 | CAGGUCCUCAC | 144 | 7511 | UGAUUAAAGUG | 1100 |
| | | | UUUAAUCCdTdT | | | AGGACCUGdTdT | |
| | D-2790 | 7512 | UCACUUUAAUC | 145 | 7513 | UGAUAGAGGAU | 1101 |
| | | | CUCUAUCCdTdT | | | UAAAGUGAdTdT | |
| | D-2791 | 7514 | CUAUCCAGAAA | 146 | 7515 | UACCGUGUUUU | 1102 |
| | | | ACACGGUCdTdT | | | CUGGAUAGdTdT | |
| | D-2792 | 7516 | UAUCCAGAAAA | 147 | 7517 | UCACCGUGUUU | 1103 |
| | | | CACGGUGCdTdT | | | UCUGGAUAdTdT | |
| | D-2793 | 7518 | AUCCAGAAAAC | 148 | 7519 | UCCACCGUGUU | 1104 |
| | | | ACGGUGGCdTdT | | | UUCUGGAUdTdT | |
| | D-2794 | 7520 | CCAGAAAACAC | 149 | 7521 | UGCCCACCGUG | 1105 |
| | | | GGUGGGCCdTdT | | | UUUUCUGGdTdT | |
| | D-2795 | 7522 | GAAAACACGGU | 150 | 7523 | UUUGGCCCACC | 1106 |
| | | | GGGCCAACdTdT | | | GUGUUUUCdTdT | |
| | D-2796 | 7524 | AAAACACGGUG | 151 | 7525 | UUUUGGCCCAC | 1107 |
| | | | GGCCAAACdTdT | | | CGUGUUUUdTdT | |
| | D-2797 | 7526 | CGGUGGGCCAA | 152 | 7527 | UUCAUCCUUUG | 1108 |
| | | | AGGAUGACdTdT | | | GCCCACCGdTdT | |
| | D-2798 | 7528 | AGGAUGAAGAG | 153 | 7529 | UCAUGCCUCUC | 1109 |
| | | | AGGCAUGCdTdT | | | UUCAUCCUdTdT | |
| | D-2799 | 7530 | AUGAAGAGAGG | 154 | 7531 | UCAACAUGCCU | 1110 |
| | | | CAUGUUGCdTdT | | | CUCUUCAUdTdT | |
| | D-2800 | 7532 | GAGAGGCAUGU | 155 | 7533 | UGUCUCCAACA | 1111 |
| | | | UGGAGACCdTdT | | | UGCCUCUCdTdT | |
| | D-2801 | 7534 | AGAGGCAUGUU | 156 | 7535 | UAGUCUCCAAC | 1112 |
| | | | GGAGACUCdTdT | | | AUGCCUCUdTdT | |
| | D-2802 | 7536 | AUGUUGGAGAC | 157 | 7537 | UUGCCCAAGUC | 1113 |
| | | | UUGGGCACdTdT | | | UCCAACAUdTdT | |
| | D-2803 | 7538 | GUUGGAGACUU | 158 | 7539 | UAUUGCCCAAG | 1114 |
| | | | GGGCAAUCdTdT | | | UCUCCAACdTdT | |
| | D-2804 | 7540 | GGAGACUUGGG | 159 | 7541 | UCACAUUGCCC | 1115 |
| | | | CAAUGUGCdTdT | | | AAGUCUCCdTdT | |
| | D-2805 | 7542 | GGCAAUGUGAC | 160 | 7543 | UGUCAGCAGUC | 1116 |
| | | | UGCUGACCdTdT | | | ACAUUGCCdTdT | |
| | D-2806 | 7544 | CAAUGUGACUG | 161 | 7545 | UUUGUCAGCAG | 1117 |
| | | | CUGACAACdTdT | | | UCACAUUGdTdT | |
| | D-2807 | 7546 | CUGACAAAGAU | 162 | 7547 | UCCACACCAUCU | 1118 |
| | | | GGUGUGGCdTdT | | | UUGUCAGdTdT | |
| | D-2808 | 7548 | UGACAAAGAUG | 163 | 7549 | UGCCACACCAUC | 1119 |
| | | | GUGUGGCCdTdT | | | UUUGUCAdTdT | |
| | D-2809 | 7550 | CUCAGGAGACC | 164 | 7551 | UAUGCAAUGGU | 1120 |
| | | | AUUGCAUCdTdT | | | CUCCUGAGdTdT | |
| | D-2810 | 7552 | UCAGGAGACCA | 165 | 7553 | UGAUGCAAUGG | 1121 |
| | | | UUGCAUCCdTdT | | | UCUCCUGAdTdT | |
| | D-2811 | 7554 | AGACCAUUGCA | 166 | 7555 | UCCAAUGAUGC | 1122 |
| | | | UCAUUGGCdTdT | | | AAUGGUCUdTdT | |
| | D-2812 | 7556 | GACCAUUGCAU | 167 | 7557 | UGCCAAUGAUG | 1123 |
| | | | CAUUGGCCdTdT | | | CAAUGGUCdTdT | |
| | D-2813 | 7558 | AUUGCAUCAUU | 168 | 7559 | UUGCGGCCAAU | 1124 |
| | | | GGCCGCACdTdT | | | GAUGCAAUdTdT | |
| | D-2814 | 7560 | CAUUGGCCGCA | 169 | 7561 | UACCAGUGUGC | 1125 |
| | | | CACUGGUCdTdT | | | GGCCAAUGdTdT | |
| | D-2815 | 7562 | CGCACACUGGU | 170 | 7563 | UAUGGACCACC | 1126 |
| | | | GGUCCAUCdTdT | | | AGUGUGCGdTdT | |
| | D-2816 | 7564 | CACACUGGUGG | 171 | 7565 | UUCAUGGACCA | 1127 |
| | | | UCCAUGACdTdT | | | CCAGUGUGdTdT | |
| | D-2817 | 7566 | ACACUGGUGGU | 172 | 7567 | UUUCAUGGACC | 1128 |
| | | | CCAUGAACdTdT | | | ACCAGUGUdTdT | |
| | D-2818 | 7568 | UGGUGGUCCAU | 173 | 7569 | UCUUUUUCAUG | 1129 |
| | | | GAAAAAGCdTdT | | | GACCACCAdTdT | |
| | D-2819 | 7570 | UGGUCCAUGAA | 174 | 7571 | UCUGCUUUUUC | 1130 |
| | | | AAAGCAGCdTdT | | | AUGGACCAdTdT | |
| | D-2820 | 7572 | AAAGCAGAUGA | 175 | 7573 | UGCCCAAGUCA | 1131 |
| | | | CUUGGGCCdTdT | | | UCUGCUUUdTdT | |
| | D-2821 | 7574 | GCAGAUGACUU | 176 | 7575 | UUUUGCCCAAG | 1132 |
| | | | GGGCAAACdTdT | | | UCAUCUGCdTdT | |
| | D-2822 | 7576 | AUGACUUGGGC | 177 | 7577 | UCACCUUUGCCC | 1133 |
| | | | AAAGGUGCdTdT | | | AAGUCAUdTdT | |
| | D-2823 | 7578 | UGACUUGGGCA | 178 | 7579 | UCCACCUUUGCC | 1134 |
| | | | AAGGUGGCdTdT | | | CAAGUCAdTdT | |
| | D-2824 | 7580 | GACUUGGGCAA | 179 | 7581 | UUCCACCUUUG | 1135 |
| | | | AGGUGGACdTdT | | | CCCAAGUCdTdT | |
| | D-2825 | 7582 | GUACAAAGACA | 180 | 7583 | UCGUUUCCUGU | 1136 |
| | | | GGAAACGCdTdT | | | CUUUGUACdTdT | |
| | D-2826 | 7584 | ACAAAGACAGG | 181 | 7585 | UAGCGUUUCCU | 1137 |
| | | | AAACGCUCdTdT | | | GUCUUUGUdTdT | |
| | D-2827 | 7586 | CAAAGACAGGA | 182 | 7587 | UCAGCGUUUCC | 1138 |
| | | | AACGCUGCdTdT | | | UGUCUUUGdTdT | |
| | D-2828 | 7588 | AGGAAACGCUG | 183 | 7589 | UCGACUUCCAG | 1139 |
| | | | GAAGUCGCdTdT | | | CGUUUCCUdTdT | |
| | D-2829 | 7590 | GUCGUUUGGCU | 184 | 7591 | UCACCACAAGCC | 1140 |
| | | | UGUGGUGCdTdT | | | AAACGACdTdT | |
| | D-2830 | 7592 | UCGUUUGGCUU | 185 | 7593 | UACACCACAAG | 1141 |
| | | | GUGGUGUCdTdT | | | CCAAACGAdTdT | |
| | D-2831 | 7594 | CGUUUGGCUUG | 186 | 7595 | UUACACCACAA | 1142 |
| | | | UGGUGUACdTdT | | | GCCAAACGdTdT | |
| | D-2832 | 7596 | GUUUGGCUUGU | 187 | 7597 | UUUACACCACA | 1143 |
| | | | GGUGUAACdTdT | | | AGCCAAACdTdT | |
| | D-2833 | 7598 | UUGGCUUGUGG | 188 | 7599 | UAAUUACACCA | 1144 |
| | | | UGUAAUUCdTdT | | | CAAGCCAAdTdT | |
| | D-2834 | 7600 | GGCUUGUGGUG | 189 | 7601 | UCCAAUUACAC | 1145 |
| | | | UAAUUGGCdTdT | | | CACAAGCCdTdT | |
| | D-2835 | 7602 | GCUUGUGGUGU | 190 | 7603 | UCCCAAUUACA | 1146 |
| | | | AAUUGGGCdTdT | | | CCACAAGCdTdT | |
| | D-2836 | 7604 | CUUGUGGUGUA | 191 | 7605 | UUCCCAAUUAC | 1147 |
| | | | AUUGGGACdTdT | | | ACCACAAGdTdT | |
| | D-2837 | 7606 | UGUGGUGUAAU | 192 | 7607 | UGAUCCCAAUU | 1148 |
| | | | UGGGAUCCdTdT | | | ACACCACAdTdT | |
| | D-2838 | 7608 | GUGGUGUAAUU | 193 | 7609 | UCGAUCCCAAU | 1149 |
| | | | GGGAUCGCdTdT | | | UACACCACdTdT | |
| | D-2839 | 7610 | UGGUGUAAUUG | 194 | 7611 | UGCGAUCCCAA | 1150 |
| | | | GGAUCGCCdTdT | | | UUACACCAdTdT | |
| | D-2840 | 7612 | GUAAUUGGGAU | 195 | 7613 | UUUGGGCGAUC | 1151 |
| | | | CGCCCAACdTdT | | | CCAAUUACdTdT | |
| | D-2841 | 7614 | UAAUUGGGAUC | 196 | 7615 | UAUUGGGCGAU | 1152 |
| | | | GCCCAAUCdTdT | | | CCCAAUUAdTdT | |
| | D-2842 | 7616 | AAUUGGGAUCG | 197 | 7617 | UUAUUGGGCGA | 1153 |
| | | | CCCAAUACdTdT | | | UCCCAAUUdTdT | |
| | D-2843 | 7618 | AUUGGGAUCGC | 198 | 7619 | UUUAUUGGGCG | 1154 |
| | | | CCAAUAACdTdT | | | AUCCCAAUdTdT | |
| | D-2844 | 7620 | UUGGGAUCGCC | 199 | 7621 | UUUUAUUGGGC | 1155 |
| | | | CAAUAAACdTdT | | | GAUCCCAAdTdT | |
| | D-2845 | 7622 | UGGGAUCGCCC | 987 | 7623 | UGUUUAUUGGG | 1156 |
| | | | AAUAAACCdTdT | | | CGAUCCCAdTdT | |
| | D-2846 | 7624 | GGGAUCGCCCA | 988 | 7625 | UUGUUUAUUGG | 1157 |
| | | | AUAAACACdTdT | | | GCGAUCCCdTdT | |
| | D-2847 | 7626 | AUCGCCCAAUA | 989 | 7627 | UGAAUGUUUAU | 1158 |
| | | | AACAUUCCdTdT | | | UGGGCGAUdTdT | |
| | D-2848 | 7628 | CCAAUAAACAU | 990 | 7629 | UCAAGGGAAUG | 1159 |
| | | | UCCCUUGCdTdT | | | UUUAUUGGdTdT | |
| | D-2849 | 7630 | CAAUAAACAUU | 991 | 7631 | UCCAAGGGAAU | 1160 |
| | | | CCCUUGGCdTdT | | | GUUUAUUGdTdT | |
| | D-2850 | 7632 | AAUAAACAUUC | 992 | 7633 | UUCCAAGGGAA | 1161 |
| | | | CCUUGGACdTdT | | | UGUUUAUUdTdT | |
| | D-2851 | 7634 | AUAAACAUUCC | 993 | 7635 | UAUCCAAGGGA | 1162 |
| | | | CUUGGAUCdTdT | | | AUGUUUAUdTdT | |
| | D-2852 | 7636 | UAAACAUUCCC | 994 | 7637 | UCAUCCAAGGG | 1163 |
| | | | UUGGAUGCdTdT | | | AAUGUUUAdTdT | |
| | D-2853 | 7638 | AAACAUUCCCU | 995 | 7639 | UACAUCCAAGG | 1164 |
| | | | UGGAUGUCdTdT | | | GAAUGUUUdTdT | |
| | D-2854 | 7640 | AACAUUCCCUU | 996 | 7641 | UUACAUCCAAG | 1165 |
| | | | GGAUGUACdTdT | | | GGAAUGUUdTdT | |
| | D-2855 | 7642 | AUUCCCUUGGA | 997 | 7643 | UGACUACAUCC | 1166 |
| | | | UGUAGUCCdTdT | | | AAGGGAAUdTdT | |
| | D-2856 | 7644 | CUUGGAUGUAG | 998 | 7645 | UCCUCAGACUA | 1167 |
| | | | UCUGAGGCdTdT | | | CAUCCAAGdTdT | |
| | D-2857 | 7646 | CUGAGGCCCCU | 999 | 7647 | UUGAGUUAAGG | 1168 |
| | | | UAACUCACdTdT | | | GGCCUCAGdTdT | |
| | D-2858 | 7648 | GAGGCCCCUUA | 1000 | 7649 | UGAUGAGUUAA | 1169 |
| | | | ACUCAUCCdTdT | | | GGGGCCUCdTdT | |
| | D-2859 | 7650 | AGGCCCCUUAA | 1001 | 7651 | UAGAUGAGUUA | 1170 |
| | | | CUCAUCUCdTdT | | | AGGGGCCUdTdT | |
| | D-2860 | 7652 | CCCCUUAACUCA | 1002 | 7653 | UAACAGAUGAG | 1171 |
| | | | UCUGUUCdTdT | | | UUAAGGGGdTdT | |
| | D-2861 | 7654 | CCCUUAACUCA | 1003 | 7655 | UUAACAGAUGA | 1172 |
| | | | UCUGUUACdTdT | | | GUUAAGGGdTdT | |
| | D-2862 | 7656 | CCUUAACUCAU | 1004 | 7657 | UAUAACAGAUG | 1173 |
| | | | CUGUUAUCdTdT | | | AGUUAAGGdTdT | |
| | D-2863 | 7658 | CUUAACUCAUC | 1005 | 7659 | UGAUAACAGAU | 1174 |
| | | | UGUUAUCCdTdT | | | GAGUUAAGdTdT | |
| | D-2864 | 7660 | UUAACUCAUCU | 1006 | 7661 | UGGAUAACAGA | 1175 |
| | | | GUUAUCCCdTdT | | | UGAGUUAAdTdT | |
| | D-2865 | 7662 | UAACUCAUCUG | 1007 | 7663 | UAGGAUAACAG | 1176 |
| | | | UUAUCCUCdTdT | | | AUGAGUUAdTdT | |
| | D-2866 | 7664 | AACUCAUCUGU | 1008 | 7665 | UCAGGAUAACA | 1177 |
| | | | UAUCCUGCdTdT | | | GAUGAGUUdTdT | |
| | D-2867 | 7666 | GUUAUCCUGCU | 1009 | 7667 | UUACAGCUAGC | 1178 |
| | | | AGCUGUACdTdT | | | AGGAUAACdTdT | |
| | D-2868 | 7668 | CUGCUAGCUGU | 1010 | 7669 | UCAUUUCUACA | 1179 |
| | | | AGAAAUGCdTdT | | | GCUAGCAGdTdT | |
| | D-2869 | 7670 | UGCUAGCUGUA | 1011 | 7671 | UACAUUUCUAC | 1180 |
| | | | GAAAUGUCdTdT | | | AGCUAGCAdTdT | |
| | D-2870 | 7672 | GCUGUAGAAAU | 1012 | 7673 | UAGGAUACAUU | 1181 |
| | | | GUAUCCUCdTdT | | | UCUACAGCdTdT | |
| | D-2871 | 7674 | CUGUAGAAAUG | 1013 | 7675 | UCAGGAUACAU | 1182 |
| | | | UAUCCUGCdTdT | | | UUCUACAGdTdT | |
| | D-2872 | 7676 | UGUAGAAAUGU | 1014 | 7677 | UUCAGGAUACA | 1183 |
| | | | AUCCUGACdTdT | | | UUUCUACAdTdT | |
| | D-2873 | 7678 | GUAGAAAUGUA | 1015 | 7679 | UAUCAGGAUAC | 1184 |
| | | | UCCUGAUCdTdT | | | AUUUCUACdTdT | |
| | D-2874 | 7680 | AAAUGUAUCCU | 1016 | 7681 | UGUUUAUCAGG | 1185 |
| | | | GAUAAACCdTdT | | | AUACAUUUdTdT | |
| | D-2875 | 7682 | GUAUCCUGAUA | 1017 | 7683 | UUAAUGUUUAU | 1186 |
| | | | AACAUUACdTdT | | | CAGGAUACdTdT | |
| | D-2876 | 7684 | UUAAACACUGU | 1018 | 7685 | UUAAGAUUACA | 1187 |
| | | | AAUCUUACdTdT | | | GUGUUUAAdTdT | |
| | D-2877 | 7686 | ACUGUAAUCUU | 1019 | 7687 | UCACUUUUAAG | 1188 |
| | | | AAAAGUGCdTdT | | | AUUACAGUdTdT | |
| | D-2878 | 7688 | CUGUAAUCUUA | 1020 | 7689 | UACACUUUUAA | 1189 |
| | | | AAAGUGUCdTdT | | | GAUUACAGdTdT | |
| | D-2879 | 7690 | UGUAAUCUUAA | 1021 | 7691 | UUACACUUUUA | 1190 |
| | | | AAGUGUACdTdT | | | AGAUUACAdTdT | |
| | D-2880 | 7692 | GUAAUCUUAAA | 1022 | 7693 | UUUACACUUUU | 1191 |
| | | | AGUGUAACdTdT | | | AAGAUUACdTdT | |
| | D-2881 | 7694 | CUUAAAAGUGU | 1023 | 7695 | UCACAAUUACA | 1192 |
| | | | AAUUGUGCdTdT | | | CUUUUAAGdTdT | |
| | D-2882 | 7696 | UACCUGUAGUG | 1024 | 7697 | UAGUUUCUCAC | 1193 |
| | | | AGAAACUCdTdT | | | UACAGGUAdTdT | |
| | D-2883 | 7698 | UUAUGAUCACU | 1025 | 7699 | UUCUUCCAAGU | 1194 |
| | | | UGGAAGACdTdT | | | GAUCAUAAdTdT | |
| | D-2884 | 7700 | AUGAUCACUUG | 1026 | 7701 | UAAUCUUCCAA | 1195 |
| | | | GAAGAUUCdTdT | | | GUGAUCAUdTdT | |
| | D-2885 | 7702 | AUCACUUGGAA | 1027 | 7703 | UACAAAUCUUC | 1196 |
| | | | GAUUUGUCdTdT | | | CAAGUGAUdTdT | |
| | D-2886 | 7704 | UGGAAGAUUUG | 1028 | 7705 | UAACUAUACAA | 1197 |
| | | | UAUAGUUCdTdT | | | AUCUUCCAdTdT | |
| | D-2887 | 7706 | UAUAAAACUCA | 1029 | 7707 | UUUUUAACUGA | 1198 |
| | | | GUUAAAACdTdT | | | GUUUUAUAdTdT | |
| | D-2888 | 7708 | AAACUCAGUUA | 1030 | 7709 | UGACAUUUUAA | 1199 |
| | | | AAAUGUCCdTdT | | | CUGAGUUUdTdT | |
| | D-2889 | 7710 | GUCUGUUUCAA | 1031 | 7711 | UCAGGUCAUUG | 1200 |
| | | | UGACCUGCdTdT | | | AAACAGACdTdT | |
| | D-2890 | 7712 | AUGACCUGUAU | 1032 | 7713 | UUGGCAAAAUA | 1201 |
| | | | UUUGCCACdTdT | | | CAGGUCAUdTdT | |
| | D-2891 | 7714 | ACCUGUAUUUU | 1033 | 7715 | UGUCUGGCAAA | 1202 |
| | | | GCCAGACCdTdT | | | AUACAGGUdTdT | |
| | D-2892 | 7716 | CCUGUAUUUUG | 1034 | 7717 | UAGUCUGGCAA | 1203 |
| | | | CCAGACUCdTdT | | | AAUACAGGdTdT | |
| | D-2893 | 7718 | UAAAUCACAGA | 1035 | 7719 | UAUACCCAUCU | 1204 |
| | | | UGGGUAUCdTdT | | | GUGAUUUAdTdT | |
| | D-2894 | 7720 | AUCACAGAUGG | 1036 | 7721 | UUUAAUACCCA | 1205 |
| | | | GUAUUAACdTdT | | | UCUGUGAUdTdT | |
| | D-2895 | 7722 | UCACAGAUGGG | 1037 | 7723 | UUUUAAUACCC | 1206 |
| | | | UAUUAAACdTdT | | | AUCUGUGAdTdT | |
| | D-2896 | 7724 | ACAGAUGGGUA | 1038 | 7725 | UAGUUUAAUAC | 1207 |
| | | | UUAAACUCdTdT | | | CCAUCUGUdTdT | |
| | D-2897 | 7726 | CAGAUGGGUAU | 1039 | 7727 | UAAGUUUAAUA | 1208 |
| | | | UAAACUUCdTdT | | | CCCAUCUGdTdT | |
| | D-2898 | 7728 | AGAUGGGUAUU | 1040 | 7729 | UCAAGUUUAAU | 1209 |
| | | | AAACUUGCdTdT | | | ACCCAUCUdTdT | |
| | D-2899 | 7730 | AUGGGUAUUAA | 1041 | 7731 | UGACAAGUUUA | 1210 |
| | | | ACUUGUCCdTdT | | | AUACCCAUdTdT | |
| | D-2900 | 7732 | UAAACUUGUCA | 1042 | 7733 | UGAAAUUCUGA | 1211 |
| | | | GAAUUUCCdTdT | | | CAAGUUUAdTdT | |
| | D-2901 | 7734 | UCAUUCAAGCC | 1043 | 7735 | UAUUCACAGGC | 1212 |
| | | | UGUGAAUCdTdT | | | UUGAAUGAdTdT | |
| | D-2902 | 7736 | CAUUCAAGCCU | 1044 | 7737 | UUAUUCACAGG | 1213 |
| | | | GUGAAUACdTdT | | | CUUGAAUGdTdT | |
| | D-2903 | 7738 | AAUAAAAACCC | 1045 | 7739 | UCCAUACAGGG | 1214 |
| | | | UGUAUGGCdTdT | | | UUUUUAUUdTdT | |
| | D-2904 | 7740 | AUAAAAACCCU | 1046 | 7741 | UGCCAUACAGG | 1215 |
| | | | GUAUGGCCdTdT | | | GUUUUUAUdTdT | |
| | D-2905 | 7742 | AACCCUGUAUG | 1047 | 7743 | UUAAGUGCCAU | 1216 |
| | | | GCACUUACdTdT | | | ACAGGGUUdTdT | |
| | D-2906 | 7744 | ACCCUGUAUGG | 1048 | 7745 | UAUAAGUGCCA | 1217 |
| | | | CACUUAUCdTdT | | | UACAGGGUdTdT | |
| | D-2907 | 7746 | GAGGCUAUUAA | 1049 | 7747 | UGAUUCUUUUA | 1218 |
| | | | AAGAAUCCdTdT | | | AUAGCCUCdTdT | |
| | D-2908 | 7748 | AAAGAAUCCAA | 1050 | 7749 | UUUUGAAUUUG | 1219 |
| | | | AUUCAAACdTdT | | | GAUUCUUUdTdT | |
| | D-2909 | 7750 | GAAUCCAAAUU | 1051 | 7751 | UUAGUUUGAAU | 1220 |
| | | | CAAACUACdTdT | | | UUGGAUUCdTdT | |
| VOYpre- | D-2910 | 7752 | CAAUGUGACUG | 1221 | 7753 | UUUGUCAGCAG | 1222 |
| 001_D- | | | CUGACAACCC | | | UCACAUUGUU | |
| 2806_Starting | | | | | | | |
| construct (18 | | | | | | | |
| native | | | | | | | |
| nucleotides and | | | | | | | |
| position 19 is | | | | | | | |
| C; 3′ terminal | | | | | | | |
| CC | | | | | | | |
| dinucleotide) | | | | | | | |
| VOYpre- | D-2911 | 7754 | CAAUGUGACUG | 1223 | 7753 | UUUGUCAGCAG | 1222 |
| 002_D- | | | CUGACAAUCC | | | UCACAUUGUU | |
| 2806_p19MMU | | | | | | | |
| (position 19 U | | | | | | | |
| to form | | | | | | | |
| mismatch) | | | | | | | |
| VOYpre- | D-2912 | 7755 | CAAUGUGACUG | 1224 | 7753 | UUUGUCAGCAG | 1222 |
| 003_D- | | | CUGACAAGCC | | | UCACAUUGUU | |
| 2806_p19GUpa | | | | | | | |
| ir (position 19 | | | | | | | |
| is G to form GU | | | | | | | |
| pair) | | | | | | | |
| VOYpre- | D-2913 | 7756 | CAAUGUGACUG | 1225 | 7753 | UUUGUCAGCAG | 1222 |
| 004_D- | | | CUGACAAACC | | | UCACAUUGUU | |
| 2806_p19AUpa | | | | | | | |
| ir (position 19 | | | | | | | |
| is A to form AU | | | | | | | |
| pair) | | | | | | | |
| VOYpre- | D-2914 | 7757 | CAAUGUGACAG | 1226 | 7753 | UUUGUCAGCAG | 1222 |
| 005_D- | | | CUGACAAACC | | | UCACAUUGUU | |
| 2806_CMM | | | | | | | |
| (central | | | | | | | |
| mismatch) | | | | | | | |
| VOYpre- | D-2915 | 7758 | CAAUGUGACUG | 1227 | 7753 | UUUGUCAGCAG | 1222 |
| 006_D- | | | CUGACAACC | | | UCACAUUGUU | |
| 2806_p19DEL | | | | | | | |
| (position 19 | | | | | | | |
| deleted) | | | | | | | |
| VOYpre- | D-2916 | 7759 | CAAUGUGACUG | 1228 | 7753 | UUUGUCAGCAG | 1222 |
| 007_D- | | | CUGACAAUCCC | | | UCACAUUGUU | |
| 2806_p19ADD | | | | | | | |
| (nucleotide | | | | | | | |
| added at | | | | | | | |
| position 19; | | | | | | | |
| addition is U; | | | | | | | |
| keep C and | | | | | | | |
| terminal CC | | | | | | | |
| dinucleotide) | | | | | | | |
| VOYpre- | D-2917 | 7752 | CAAUGUGACUG | 1221 | 7753 | UUUGUCAGCAG | 1222 |
| 008_D- | | | CUGACAACCC | | | UCACAUUGUU | |
| 2806_Uloop | | | | | | | |
| VOYpre- | D-2918 | 7752 | CAAUGUGACUG | 1221 | 7753 | UUUGUCAGCAG | 1222 |
| 009_D- | | | CUGACAACCC | | | UCACAUUGUU | |
| 2806_AUloop | | | | | | | |
| VOYpre- | D-2919 | 7760 | CAAUGUGACUG | 1229 | 7753 | UUUGUCAGCAG | 1222 |
| 010_D- | | | CUGACAACAC | | | UCACAUUGUU | |
| 2806_mir-22- | | | | | | | |
| loop | | | | | | | |
| VOYmiR- | D-2923 | 7752 | CAAUGUGACUG | 1221 | 7753 | UUUGUCAGCAG | 1222 |
| 101_pre-001 | | | CUGACAACCC | | | UCACAUUGUU | |
| hsa-mir-155; D- | | | | | | | |
| 2806 | | | | | | | |
| VOYmiR- | D-2924 | 7752 | CAAUGUGACUG | 1221 | 7753 | UUUGUCAGCAG | 1222 |
| 102_pre-001 | | | CUGACAACCC | | | UCACAUUGUU | |
| Engineered; D- | | | | | | | |
| 2806; let-7b | | | | | | | |
| stem | | | | | | | |
| VOYmiR- | D-2925 | 7754 | CAAUGUGACUG | 1223 | 7753 | UUUGUCAGCAG | 1222 |
| 103_pre-002 | | | CUGACAAUCC | | | UCACAUUGUU | |
| Engineered; D- | | | | | | | |
| 2806_p19MMU; | | | | | | | |
| let-7b stem | | | | | | | |
| VOYmiR- | D-2926 | 7755 | CAAUGUGACUG | 1224 | 7753 | UUUGUCAGCAG | 1222 |
| 104_pre-003 | | | CUGACAAGCC | | | UCACAUUGUU | |
| Engineered; D- | | | | | | | |
| 2806_p19GUpair; | | | | | | | |
| let-7b stem | | | | | | | |
| VOYmiR- | D-2927 | 7756 | CAAUGUGACUG | 1225 | 7753 | UUUGUCAGCAG | 1222 |
| 105_pre-004 | | | CUGACAAACC | | | UCACAUUGUU | |
| Engineered; D- | | | | | | | |
| 2806_p19AUpair; | | | | | | | |
| let-7b stem | | | | | | | |
| VOYmiR- | D-2928 | 7757 | CAAUGUGACAG | 1226 | 7753 | UUUGUCAGCAG | 1222 |
| 106_pre-005 | | | CUGACAAACC | | | UCACAUUGUU | |
| Engineered; D- | | | | | | | |
| 2806_CMM; | | | | | | | |
| let-7b stem | | | | | | | |
| VOYmiR- | D-2929 | 7758 | CAAUGUGACUG | 1227 | 7753 | UUUGUCAGCAG | 1222 |
| 107_pre-006 | | | CUGACAACC | | | UCACAUUGUU | |
| Engineered; D- | | | | | | | |
| 2806_p19DEL; | | | | | | | |
| let-7b stem | | | | | | | |
| VOYmiR- | D-2930 | 7765 | CAAUGUGACUG | 1234 | 7753 | UUUGUCAGCAG | 1222 |
| 108_pre-007 | | | CUGACAAUCCC | | | UCACAUUGUU | |
| Engineered; D- | | | | | | | |
| 2806_p19ADD; | | | | | | | |
| let-7b stem | | | | | | | |
| VOYmiR- | D-2931 | 7752 | CAAUGUGACUG | 1221 | 7753 | UUUGUCAGCAG | 1222 |
| 109_pre-008 | | | CUGACAACCC | | | UCACAUUGUU | |
| Engineered; D- | | | | | | | |
| 2806_Uloop; | | | | | | | |
| let-7b stem | | | | | | | |
| VOYmiR- | D-2932 | 7752 | CAAUGUGACUG | 1221 | 7753 | UUUGUCAGCAG | 1222 |
| 110_pre-009 | | | CUGACAACCC | | | UCACAUUGUU | |
| Engineered; D- | | | | | | | |
| 2806_AUloop; | | | | | | | |
| let-7b stem | | | | | | | |
| VOYmiR- | D-2933 | 7760 | CAAUGUGACUG | 1229 | 7753 | UUUGUCAGCAG | 1222 |
| 111_pre-010 | | | CUGACAACAC | | | UCACAUUGUU | |
| Engineered; D- | | | | | | | |
| 2806_mir-22- | | | | | | | |
| loop; let-7b | | | | | | | |
| stem | | | | | | | |
| VOYmiR- | D-2934 | 7752 | CAAUGUGACUG | 1221 | 7753 | UUUGUCAGCAG | 1222 |
| 112_pre-001 | | | CUGACAACCC | | | UCACAUUGUU | |
| Engineered; | | | | | | | |
| PD; D-2806; | | | | | | | |
| let-7b basal- | | | | | | | |
| stem instability | | | | | | | |
| VOYmiR- | D-2935 | 7754 | CAAUGUGACUG | 1223 | 7753 | UUUGUCAGCAG | 1222 |
| 113_pre-002 | | | CUGACAAUCC | | | UCACAUUGUU | |
| Engineered; D- | | | | | | | |
| 2806_p19MMU; | | | | | | | |
| let-7b basal- | | | | | | | |
| stem instability | | | | | | | |
| VOYmiR- | D-2936 | 7757 | CAAUGUGACAG | 1226 | 7753 | UUUGUCAGCAG | 1222 |
| 114_pre-005 | | | CUGACAAACC | | | UCACAUUGUU | |
| Engineered; D- | | | | | | | |
| 2806_CMM; | | | | | | | |
| let-7b basal- | | | | | | | |
| stem instability | | | | | | | |
| VOYmiR- | D-2937 | 7760 | CAAUGUGACUG | 1229 | 7753 | UUUGUCAGCAG | 1222 |
| 115_pre-010 | | | CUGACAACAC | | | UCACAUUGUU | |
| Engineered; D- | | | | | | | |
| 2806_mir-22- | | | | | | | |
| loop; let-7b | | | | | | | |
| basal-stem | | | | | | | |
| instability | | | | | | | |
| VOYmiR- | D-2938 | 7755 | CAAUGUGACUG | 1224 | 7753 | UUUGUCAGCAG | 1222 |
| 116_pre-003 | | | CUGACAAGCC | | | UCACAUUGUU | |
| Engineered; D- | | | | | | | |
| 2806_p19GUpair; | | | | | | | |
| let-7b basal- | | | | | | | |
| stem instability | | | | | | | |
| VOYmiR- | D-2939 | 7766 | CGACGAAGGCC | 1235 | 7767 | UCGCACACGGCC | 1236 |
| 117_pre-001 | | | GUGUGCGCCC | | | UUCGUCGUU | |
| Engineered; D- | | | | | | | |
| 2757; let-7b | | | | | | | |
| stem | | | | | | | |
| VOYmiR- | D-2940 | 7768 | UGACUUGGGCA | 1237 | 7769 | UCCACCUUUGCC | 1238 |
| 118_pre-001 | | | AAGGUGGCCC | | | CAAGUCAUU | |
| Engineered; D- | | | | | | | |
| 2823; let-7b | | | | | | | |
| stem | | | | | | | |
| VOYmiR- | D-2941 | 7770 | AACUCAUCUGU | 1239 | 7771 | UCAGGAUAACA | 1240 |
| 119_pre-001 | | | UAUCCUGCCC | | | GAUGAGUUUU | |
| Engineered; D- | | | | | | | |
| 2866; let-7b | | | | | | | |
| stem | | | | | | | |
| VOYpre- | D-2920 | 7761 | UUUGUCAGCAG | 1230 | 7762 | CAAUGUGACUG | 1231 |
| 011_D- | | | UCACAUUGUC | | | CUGACAAAUC | |
| 2806_passenger- | | | | | | | |
| guide strand | | | | | | | |
| swap with | | | | | | | |
| terminal 3′ C | | | | | | | |
| on passenger | | | | | | | |
| strand | | | | | | | |
| VOYpre- | D-2921 | 7761 | UUUGUCAGCAG | 1230 | 7763 | CAAUGUGACUG | 1232 |
| 012_D-2806 | | | UCACAUUGUC | | | CUGACAAUUC | |
| passenger-guide | | | | | | | |
| strand swap | | | | | | | |
| with terminal 3′ | | | | | | | |
| C on passenger | | | | | | | |
| strand | | | | | | | |
| VOYpre- | D-2922 | 7764 | UUUGUCAGCAG | 1233 | 7762 | CAAUGUGACUG | 1231 |
| 013_D-2806 | | | UCACAUUGAC | | | CUGACAAAUC | |
| passenger-guide | | | | | | | |
| strand swap | | | | | | | |
| with terminal 3″ | | | | | | | |
| C on passenger | | | | | | | |
| strand | | | | | | | |
| VOYmiR-127 | D-2942 | 7752 | CAAUGUGACUG | 1221 | 7753 | UUUGUCAGCAG | 1222 |
| | | | CUGACAACCC | | | UCACAUUGUU | |
| VOYmiR- | D-2943 | 7772 | CCCCUUAACUCA | 1241 | 7773 | UAACAGAUGAG | 2363 |
| 102.860 | | | UCUGUUCCC | | | UUAAGGGGUU | |
| VOYmiR102.861 | D-2944 | 7774 | CCCUUAACUCA | 1243 | 7775 | UUAACAGAUGA | 2365 |
| | | | UCUGUUACCC | | | GUUAAGGGUU | |
| VOYmiR- | D-2945 | 7776 | AACUCAUCUGU | 1245 | 7771 | UCAGGAUAACA | 1240 |
| 102.866 | | | UAUCUUGCCC | | | GAUGAGUUUU | |
| VOYmiR- | D-2946 | 7777 | GCUGUGGAAAU | 1246 | 7778 | UAGGAUACAUU | 1247 |
| 102.870 | | | GUAUCUUCCC | | | UCUACAGCUU | |
| VOYmiR- | D-2947 | 7779 | UGACUUGGGCA | 1248 | 7769 | UCCACCUUUGCC | 1238 |
| 102.823 | | | AAGGUGAGCC | | | CAAGUCAUU | |
| VOYmiR- | D-2948 | 7780 | CCCCUUAACUCA | 1249 | 7773 | UAACAGAUGAG | 2363 |
| 104.860 | | | UCUGUUGCC | | | UUAAGGGGUU | |
| VOYmiR- | D-2949 | 7781 | CCCUUAACUCA | 1250 | 7775 | UUAACAGAUGA | 2365 |
| 104.861 | | | UCUGUUAGCC | | | GUUAAGGGUU | |
| VOYmiR- | D-2950 | 7782 | AACUCAUCUGU | 1251 | 7771 | UCAGGAUAACA | 1240 |
| 104.866 | | | UAUCUUAGCC | | | GAUGAGUUUU | |
| VOYmiR- | D-2951 | 7783 | GCUGUGGAAAU | 1252 | 7778 | UAGGAUACAUU | 1247 |
| 104.870 | | | GUAUCUUGCC | | | UCUACAGCUU | |
| VOYmiR- | D-2952 | 7784 | UGACUUGGGCA | 1253 | 7769 | UCCACCUUUGCC | 1238 |
| 104.823 | | | AAGGUAGGCC | | | CAAGUCAUU | |
| VOYmiR- | D-2953 | 7772 | CCCCUUAACUCA | 1241 | 7773 | UAACAGAUGAG | 2363 |
| 109.860 | | | UCUGUUCCC | | | UUAAGGGGUU | |
| VOYmiR- | D-2954 | 7774 | CCCUUAACUCA | 1243 | 7775 | UUAACAGAUGA | 2365 |
| 104.861 | | | UCUGUUACCC | | | GUUAAGGGUU | |
| VOYmiR- | D-2955 | 7776 | AACUCAUCUGU | 1245 | 7771 | UCAGGAUAACA | 1240 |
| 104.866 | | | UAUCUUGCCC | | | GAUGAGUUUU | |
| VOYmiR- | D-2956 | 7777 | GCUGUGGAAAU | 1246 | 7778 | UAGGAUACAUU | 1247 |
| 109.870 | | | GUAUCUUCCC | | | UCUACAGCUU | |
| VOYmiR- | D-2957 | 7779 | UGACUUGGGCA | 1248 | 7769 | UCCACCUUUGCC | 1238 |
| 109.823 | | | AAGGUGAGCC | | | CAAGUCAUU | |
| VOYmiR- | D-2958 | 7785 | CCCCUUAACACA | 1254 | 7773 | UAACAGAUGAG | 2363 |
| 114.860 | | | UCUGUUACC | | | UUAAGGGGUU | |
| VOYmiR- | D-2959 | 7786 | CCCUUAACUGA | 2376 | 7775 | UUAACAGAUGA | 2365 |
| 114.861 | | | UCUGUUAACC | | | GUUAAGGGUU | |
| VOYmiR- | D-2960 | 7787 | AACUCAUCUCU | 1256 | 7771 | UCAGGAUAACA | 1240 |
| 114.866 | | | UAUCUUGCCC | | | GAUGAGUUUU | |
| VOYmiR- | D-2961 | 7788 | GCUGUGGAAUU | 1257 | 7778 | UAGGAUACAUU | 1247 |
| 114.870 | | | GUAUCUUGCC | | | UCUACAGCUU | |
| VOYmiR- | D-2962 | 7789 | UGACUUGGGGA | 1258 | 7769 | UCCACCUUUGCC | 1238 |
| 114.823 | | | AAGGUGAGCC | | | CAAGUCAUU | |
| VOYmiR- | D-2963 | 7780 | CCCCUUAACUCA | 1249 | 7773 | UAACAGAUGAG | 2363 |
| 116.860 | | | UCUGUUGCC | | | UUAAGGGGUU | |
| VOYmiR- | D-2964 | 7781 | CCCUUAACUCA | 1250 | 7775 | UUAACAGAUGA | 2365 |
| 116.861 | | | UCUGUUAGCC | | | GUUAAGGGUU | |
| VOYmiR- | D-2965 | 7790 | AACUCAUCUGU | 1259 | 7771 | UCAGGAUAACA | 1240 |
| 116.866 | | | UAUCUUGGCC | | | GAUGAGUUUU | |
| VOYmiR- | D-2966 | 7783 | GCUGUGGAAAU | 1252 | 7778 | UAGGAUACAUU | 1247 |
| 116.870 | | | GUAUCUUGCC | | | UCUACAGCUU | |
| VOYmiR- | D-2967 | 7784 | UGACUUGGGCA | 1253 | 7769 | UCCACCUUUGCC | 1238 |
| 116.823 | | | AAGGUAGGCC | | | CAAGUCAUU | |
| VoymiR- | D-2968 | 7791 | CCCCUUAACUCA | 2381 | 7773 | UAACAGAUGAG | 2363 |
| 127.860 | | | UUUGUUCCC | | | UUAAGGGGUU | |
| VoymiR- | D-2969 | 7774 | CCCUUAACUCA | 1243 | 7775 | UUAACAGAUGA | 2365 |
| 127.861 | | | UCUGUUACCC | | | GUUAAGGGUU | |
| VoymiR- | D-2970 | 7776 | AACUCAUCUGU | 1245 | 7771 | UCAGGAUAACA | 1240 |
| 127.866 | | | UAUCUUGCCC | | | GAUGAGUUUU | |
| VoymiR- | D-2971 | 7777 | GCUGUGGAAAU | 1246 | 7778 | UAGGAUACAUU | 1247 |
| 127.870 | | | GUAUCUUCCC | | | UCUACAGCUU | |
| VoymiR- | D-2972 | 7792 | UGACUUGGGCA | 1261 | 7769 | UCCACCUUUGCC | 1238 |
| 127.823 | | | AAGGUAGCCC | | | CAAGUCAUU | |
| VOYmiR-120 | D-2973 | 7793 | CAAUGUGACUG | 1263 | 7794 | UUUGUCAGCAG | 1264 |
| | | | CUGACAAA | | | UCACAUUGUC | |
| dVOYmiR- | D-2974 | 7795 | GCAGGUCCUCA | 1265 | 7796 | GAUUAAAGUGA | 1266 |
| 102.788 | | | CUUUAAUGCC | | | GGACCUGCUU | |
| dVOYmiR- | D-2975 | 7797 | GGCAAUGUGAC | 1267 | 7798 | UGUCAGCAGUC | 1268 |
| 102.805 | | | UGCUGACCCC | | | ACAUUGCCUU | |
| dVOYmiR- | D-2976 | 7799 | GCAGGUCCUCA | 1269 | 7796 | GAUUAAAGUGA | 1266 |
| 104.788 | | | CUUUAAUUCC | | | GGACCUGCUU | |
| dVOYmiR- | D-2977 | 7800 | GGCAAUGUGAC | 1270 | 7798 | UGUCAGCAGUC | 1268 |
| 104.805 | | | UGCUGAUGCC | | | ACAUUGCCUU | |
| dVOYmiR- | D-2978 | 7801 | GCAGGUCCUCA | 1271 | 7796 | GAUUAAAGUGA | 1266 |
| 109.788 | | | CUUUAAUCCC | | | GGACCUGCUU | |
| dVOYmiR- | D-2979 | 7802 | GGCAAUGUGAC | 1272 | 7798 | UGUCAGCAGUC | 1268 |
| 109.805 | | | UGCUGAUACC | | | ACAUUGCCUU | |
| dVOYmiR- | D-2980 | 7803 | GCAGGUCCUGA | 1273 | 7796 | GAUUAAAGUGA | 1266 |
| 114.788 | | | CUUUAAUCCC | | | GGACCUGCUU | |
| dVOYmiR- | D-2981 | 7804 | GGCAAUGUGUC | 1274 | 7798 | UGUCAGCAGUC | 1268 |
| 114.805 | | | UGCUGAUACC | | | ACAUUGCCUU | |
| dVOYmiR- | D-2982 | 7801 | GCAGGUCCUCA | 1271 | 7796 | GAUUAAAGUGA | 1266 |
| 116.788 | | | CUUUAAUCCC | | | GGACCUGCUU | |
| dVOYmiR- | D-2983 | 7802 | GGCAAUGUGAC | 1272 | 7798 | UGUCAGCAGUC | 1268 |
| 116.805 | | | UGCUGAUACC | | | ACAUUGCCUU | |
| dVoymiR- | D-2984 | 7801 | GCAGGUCCUCA | 1271 | 7805 | GAUUAAAGUGA | 1275 |
| 127.788 | | | CUUUAAUCCC | | | GGACCUGCUUU | |
| dVoymiR- | D-2985 | 7802 | GGCAAUGUGAC | 1272 | 7806 | UGUCAGCAGUC | 1276 |
| 127.805 | | | UGCUGAUACC | | | ACAUUGCCUUU | |
| | D-4009 | S-4000 | GUCGUUUGGCU | 1283 | A-4000 | UCACCACAAGCC | 1277 |
| | | | UGUGGUGGCU | | | AAACGACUU | |
| | D-4010 | S-4001 | CAGGUCCUCAC | 1284 | A-4001 | UGAUUAAAGUG | 1278 |
| | | | UUUAAUCGCU | | | AGGACCUGUU | |
| | D-4011 | S-4002 | GCAGGUCCUCA | 1285 | A-4002 | UAUUAAAGUGA | 2507 |
| | | | CUUUAAUGCC | | | GGACCUGCUU | |
| | D-4012 | S-4003 | GCAGGUCCUCA | 2525 | A-4002 | UAUUAAAGUGA | 2507 |
| | | | CUUUAAUGCU | | | GGACCUGCUU | |
| | D-4013 | S-4004 | UCGUUUGGCUU | 1287 | A-4003 | UACACCACAAG | 1280 |
| | | | GUGGUGUGCU | | | CCAAACGAUU | |
| | D-4015 | S-4006 | GUCGUUUGGCU | 1288 | A-4005 | UCACCACAAGCC | 1281 |
| | | | UGUGGUGGCC | | | AAACGACUUU | |
| | D-4016 | S-4007 | CAGGUCCUCAC | 1289 | A-4001 | UGAUUAAAGUG | 1278 |
| | | | UUUAAUCGCC | | | AGGACCUGUU | |
| | D-4017 | S-4008 | UCGUUUGGCUU | 1290 | A-4003 | UACACCACAAG | 1280 |
| | | | GUGGUGUGCC | | | CCAAACGAUU | |
| | D-4018 | S-4009 | GGCAAUGUGAC | 1291 | A-4006 | UGCCAGCAGUC | 1282 |
| | | | UGCUGGUGCC | | | ACAUUGCCUU | |
| | D-4019 | S-4010 | GGCAAUGUGAC | 1292 | A-4006 | UGCCAGCAGUC | 1282 |
| | | | UGCUGGUACC | | | ACAUUGCCUU | |
| | D-4020 | S-4011 | GGCAAUGUGUC | 1293 | A-4006 | UGCCAGCAGUC | 1282 |
| | | | UGCUGGUACC | | | ACAUUGCCUU | |
| | D-4021 | S-4012 | GGCAAUGUGAC | 1294 | A-4006 | UGCCAGCAGUC | 1281 |
| | | | UGCUGGCCCC | | | ACAUUGCCUU | |

In some embodiments, the dTdT nucleotides of the sense strand and/or the antisense strand of the siRNA may be replaced with any two nucleotides (e.g., UU, UC, AA, AG, AC, etc.). Alternatively, the dTdT nucleotides of the sense strand and/or the antisense strand may be replaced with a single nucleotide (e.g., U, A, C, or G).

In some embodiments, the siRNA molecules of the present disclosure can be encoded by a vector, e.g., plasmid vector, a viral genome (e.g., an AAV viral genome), or another nucleic acid expression vector for delivery to a cell. In some embodiments, a siRNA molecule is encoded by a viral genome of an AAV particle.

DNA expression plasmids can be used to stably express the siRNA duplexes or dsRNA of the present disclosure in cells and achieve long-term inhibition of the target gene expression. In one aspect, the sense and antisense strands of a siRNA duplex are typically linked by a short spacer sequence leading to the expression of a stem-loop structure termed short hairpin RNA (shRNA). The hairpin is recognized and cleaved by Dicer, thus generating mature siRNA molecules.

In some embodiments, the sense and antisense strands of a siRNA duplex may be linked by a short spacer sequence, which may optionally be linked to additional flanking sequence, leading to the expression of a flanking arm-stem-loop structure termed primary microRNA (pri-miRNA). The pri-miRNA may be recognized and cleaved by Drosha and Dicer, and thus generate mature siRNA molecules.

In other embodiments, the siRNA molecules of the present disclosure can be encoded in plasmid vectors, AAV particles, viral genome or other nucleic acid expression vectors for delivery to a cell.

In some embodiments, a siRNA for inhibiting expression of SOD1 is provided, wherein said siRNA comprises a sense strand and an antisense strand, the antisense strand comprising a region of complementarity to a SOD1 RNA transcript, which antisense strand comprises at least 15 (e.g., at least 16, 17, 18, 19, 20, 21, 22, or 23) contiguous nucleotides differing by no more than 3 (e.g., by no more than 0, 1 or 2) nucleotides from an antisense sequence listed in any one of the duplexes in Table 10 or 14. In one embodiment, the antisense sequence comprises at least 15 (e.g., at least 16, 17, 18, 19, 20, 21, 22, or 23) contiguous nucleotides differing by no more than 3 (e.g., by no more than 0, 1 or 2) nucleotides from the antisense sequence of SEQ ID NO: 2507, 2363 or 2365.

In some embodiments, the antisense strand comprises at least 15, 16, 17, 18, 19, 20, or 21 contiguous nucleotides differing by no more than 3, 2, 1, or 0 nucleotides from SEQ ID NO: 2507. In some embodiments, the antisense strand comprises at least 18, 19, 20, or 21 contiguous nucleotides differing by no more than 3, 2, 1, or 0 nucleotides from SEQ ID NO: 2507. In some embodiments, the antisense strand comprises at least 18 contiguous nucleotides of SEQ ID NO: 2507. In some embodiments, the antisense strand comprises at least 19 contiguous nucleotides of SEQ ID NO: 2507. In some embodiments, the antisense strand comprises at least 20 contiguous nucleotides of SEQ ID NO: 2507. In some embodiments, the antisense strand sequence comprises nucleotides 1-15, 1-16, 1-17, 1-18, 1-19, or 1-20 from the nucleotide sequence of SEQ ID NO: 2507. In some embodiments, the antisense strand sequence comprises the nucleotide sequence of SEQ ID NO: 2507. In some embodiments, the sense strand comprises at least 15, 16, 17, 18, 19, 20, or 21 contiguous nucleotides differing by no more than 3, 2, 1, or 0 nucleotides from SEQ ID NO: 2525. In some embodiments, the sense strand comprises at least 18, 19, 20, or 21 contiguous nucleotides differing by no more than 3, 2, 1, or 0 nucleotides from SEQ ID NO: 2525. In some embodiments, the sense strand comprises at least 18 contiguous nucleotides of SEQ ID NO: 2525. In some embodiments, the sense strand comprises at least 19 contiguous nucleotides of SEQ ID NO: 2525. In some embodiments, the sense strand comprises at least 20 contiguous nucleotides of SEQ ID NO: 2525. In some embodiments, the sense strand sequence comprises nucleotides 1-15, 1-16, 1-17, 1-18, 1-19, or 1-20 from the nucleotide sequence of SEQ ID NO: 2525. In some embodiments, the sense strand sequence comprises the nucleotide sequence of SEQ ID NO: 2525.

In some embodiments, the antisense strand comprises at least 15, 16, 17, 18, 19, 20, or 21 contiguous nucleotides differing by no more than 3, 2, 1, or 0 nucleotides from SEQ ID NO: 2507; and the sense strand comprises at least 15, 16, 17, 18, 19, 20, or 21 contiguous nucleotides differing by no more than 3, 2, 1, or 0 nucleotides from SEQ ID NO: 2525. In some embodiments, the antisense strand comprises at least 18, 19, 20, or 21 contiguous nucleotides differing by no more than 3, 2, 1, or 0 nucleotides from SEQ ID NO: 2507; and the sense strand comprises at least 18, 19, 20, or 21 contiguous nucleotides differing by no more than 3, 2, 1, or 0 nucleotides from SEQ ID NO: 2525. In some embodiments, the antisense strand comprises at least 18 contiguous nucleotides of SEQ ID NO: 2507; and the sense strand comprises at least 18 contiguous nucleotides of SEQ ID NO: 2525. In some embodiments, the antisense strand comprises at least 19 contiguous nucleotides of SEQ ID NO: 2507; and the sense strand comprises at least 19 contiguous nucleotides of SEQ ID NO: 2525. In some embodiments, the antisense strand comprises at least 20 contiguous nucleotides of SEQ ID NO: 2507; and the sense strand comprises at least 20 contiguous nucleotides of SEQ ID NO: 2525. In some embodiments, the antisense strand sequence comprises nucleotides 1-15, 1-16, 1-17, 1-18, 1-19, or 1-20 from the nucleotide sequence of SEQ ID NO: 2507; and the sense strand sequence comprises nucleotides 1-15, 1-16, 1-17, 1-18, 1-19, or 1-20 from the nucleotide sequence of SEQ ID NO: 2525. In some embodiments, the antisense strand sequence comprises the nucleotide sequence of SEQ ID NO: 2507; and the sense strand sequence comprises the nucleotide sequence of SEQ ID NO: 2525.

In some embodiments, the antisense strand comprises at least 15, 16, 17, 18, 19, 20, or 21 contiguous nucleotides differing by no more than 3, 2, 1, or 0 nucleotides from SEQ ID NO: 2363. In some embodiments, the antisense strand comprises at least 18, 19, 20, or 21 contiguous nucleotides differing by no more than 3, 2, 1, or 0 nucleotides from SEQ ID NO: 2363. In some embodiments, the antisense strand comprises at least 18 contiguous nucleotides of SEQ ID NO: 2363. In some embodiments, the antisense strand comprises at least 19 contiguous nucleotides of SEQ ID NO: 2363. In some embodiments, the antisense strand comprises at least 20 contiguous nucleotides of SEQ ID NO: 2363. In some embodiments, the antisense strand sequence comprises nucleotides 1-15, 1-16, 1-17, 1-18, 1-19, or 1-20 from the nucleotide sequence of SEQ ID NO: 2363. In some embodiments, the antisense strand sequence comprises the nucleotide sequence of SEQ ID NO: 2363. In some embodiments, the sense strand comprises at least 15, 16, 17, 18, 19, 20, or 21 contiguous nucleotides differing by no more than 3, 2, 1, or 0 nucleotides from SEQ ID NO: 2381. In some embodiments, the sense strand comprises at least 18, 19, 20, or 21 contiguous nucleotides differing by no more than 3, 2, 1, or 0 nucleotides from SEQ ID NO: 2381. In some embodiments, the sense strand comprises at least 18 contiguous nucleotides of SEQ ID NO: 2381. In some embodiments, the sense strand comprises at least 19 contiguous nucleotides of SEQ ID NO: 2381. In some embodiments, the sense strand comprises at least 20 contiguous nucleotides of SEQ ID NO: 2381. In some embodiments, the sense strand sequence comprises nucleotides 1-15, 1-16, 1-17, 1-18, 1-19, or 1-20 from the nucleotide sequence of SEQ ID NO: 2381. In some embodiments, the sense strand sequence comprises the nucleotide sequence of SEQ ID NO: 2381.

In some embodiments, the antisense strand comprises at least 15, 16, 17, 18, 19, 20, or 21 contiguous nucleotides differing by no more than 3, 2, 1, or 0 nucleotides from SEQ ID NO: 2363; and the sense strand comprises at least 15, 16, 17, 18, 19, 20, or 21 contiguous nucleotides differing by no more than 3, 2, 1, or 0 nucleotides from SEQ ID NO: 2381. In some embodiments, the antisense strand comprises at least 18, 19, 20, or 21 contiguous nucleotides differing by no more than 3, 2, 1, or 0 nucleotides from SEQ ID NO: 2363; and the sense strand comprises at least 18, 19, 20, or 21 contiguous nucleotides differing by no more than 3, 2, 1, or 0 nucleotides from SEQ ID NO: 2381. In some embodiments, the antisense strand comprises at least 18 contiguous nucleotides of SEQ ID NO: 2363; and the sense strand comprises at least 18 contiguous nucleotides of SEQ ID NO: 2381. In some embodiments, the antisense strand comprises at least 19 contiguous nucleotides of SEQ ID NO: 2363; and the sense strand comprises at least 19 contiguous nucleotides of SEQ ID NO: 2381. In some embodiments, the antisense strand comprises at least 20 contiguous nucleotides of SEQ ID NO: 2363; and the sense strand comprises at least 20 contiguous nucleotides of SEQ ID NO: 2381. In some embodiments, the antisense strand sequence comprises nucleotides 1-15, 1-16, 1-17, 1-18, 1-19, or 1-20 from the nucleotide sequence of SEQ ID NO: 2363; and the sense strand sequence comprises nucleotides 1-15, 1-16, 1-17, 1-18, 1-19, or 1-20 from the nucleotide sequence of SEQ ID NO: 2381. In some embodiments, the antisense strand sequence comprises the nucleotide sequence of SEQ ID NO: 2363; and the sense strand sequence comprises the nucleotide sequence of SEQ ID NO: 2381.

Additional sense and antisense strand sequences useful for the siRNA duplexes of the present application are disclosed in PCT Patent Application Publications WO2016077687, WO2019079240, WO2018204786, WO2019079242, WO 2020010042 and WO2020223296, which are hereby incorporated by reference in their entirety.

In some embodiments, the siRNA duplexes or encoded dsRNA of the present disclosure suppress (or degrade) the SOD1 mRNA. Accordingly, the siRNA duplexes or encoded dsRNA can be used to substantially inhibit SOD1 in a cell, for example a neuron.

The expression of SOD1 may be assessed based on the level of expression of SOD1 mRNA, SOD1 protein, or the level of another parameter functionally linked to the level of expression of SOD1. In some embodiments, the expression of SOD1 is inhibited by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, the siRNA has an IC50 in the range of 0.001-0.01 nM, 0.001-0.10 nM, 0.001-1.0 nM, 0.001-10 nM, 0.01-0.05 nM, 0.01-0.50 nM, 0.02-0.60 nM, 0.01-1.0 nM, 0.01-1.5 nM, 0.01-10 nM. The IC₅₀ value may be normalized relative to an appropriate control value, e.g., the IC₅₀ of a non-targeting siRNA. Accordingly, the protein product of SOD1 may be inhibited. In some embodiments, the siRNA duplexes or encoded dsRNA of the present disclosure suppress (or degrade) the target mRNA in spinal cord motor neurons (e.g., RNA in vg+ cells of motor neuron morphology).

According to the present disclosure, the siRNA molecules are designed and tested for their ability in reducing SOD1 mRNA levels in cultured cells. Such siRNA molecules may form a duplex such as, but not limited to, include those listed in Table 10 or 14. As a non-limiting example, the siRNA duplexes may be siRNA duplex ID D-4012, D-2968, or D-2959.

In some embodiments, the siRNA duplex comprises: (i) a sense strand sequence comprising SEQ ID NO: 2525, and an antisense strand sequence comprising SEQ ID NO: 2507; (ii) a sense strand sequence comprising SEQ ID NO: 2376, and an antisense strand sequence comprising SEQ ID NO: 2365; or (iii) a sense strand sequence comprising SEQ ID NO: 2381, and an antisense strand sequence comprising SEQ ID NO: 2363.

In some embodiments, the sense strand sequence and the antisense strand sequence comprise a region of complementarity, wherein the region of complementarity is between 17-20 nucleotides in length.

In certain embodiments, the siRNA molecules comprise a miRNA seed match for SOD1 located in the guide strand. In another embodiment, the siRNA molecules comprise a miRNA seed match for SOD1 located in the passenger strand. In yet another embodiment, the siRNA duplexes or encoded dsRNA targeting SOD1 gene do not comprise a seed match for SOD1 located in the guide or passenger strand.

In certain embodiments, the siRNA duplex target SOD1 is designed so there is no miRNA seed match for the sense or antisense sequence to the non-SOD1 sequence.

In some embodiments, the siRNA duplexes or encoded dsRNA targeting SOD1 may have almost no significant full-length off target effects for the guide strand, and/or for the passenger strand.

In certain embodiments, the siRNA molecules have a high guide strand activity and low passenger strand activity in vitro. The target knock-down (KD) by the guide strand may be at least 40%, 50%, 60%, 65%, 70%, 75%, 80%, 85%, 90%, 95%, 99%, 99.5% or 100%. As a non-limiting example, the target knock-down may persist for a period of time of at least 4 weeks, at least 8 weeks, at least 12 weeks, or more. In some embodiments, the highest knock-down from delivery of the siRNA molecules is seen around the injection site(s). In some embodiments, knock-down is seen in the ventral horn and around the injection site(s) after delivery of the siRNA molecules.

In some embodiments, the guide to passenger (G: P) (also referred to as the antisense to sense) strand ratio is 1:10, 1:9, 1:8, 1:7, 1:6, 1:5, 1:4, 1:3, 1:2, 1;1, 2:10, 2:9, 2:8, 2:7, 2:6, 2:5, 2:4, 2:3, 2:2, 2:1, 3:10, 3:9, 3:8, 3:7, 3:6, 3:5, 3:4, 3:3, 3:2, 3:1, 4:10, 4:9, 4:8, 4:7, 4:6, 4:5, 4:4, 4:3, 4:2, 4:1, 5:10, 5:9, 5:8, 5:7, 5:6, 5:5, 5:4, 5:3, 5:2, 5:1, 6:10, 6:9, 6:8, 6:7, 6:6, 6:5, 6:4, 6:3, 6:2, 6:1, 7:10, 7:9, 7:8, 7:7, 7:6, 7:5, 7:4, 7:3, 7:2, 7:1, 8:10, 8:9, 8:8, 8:7, 8:6, 8:5, 8:4, 8:3, 8:2, 8:1, 9:10, 9:9, 9:8, 9:7, 9:6, 9:5, 9:4, 9:3, 9:2, 9:1, 10:10, 10:9, 10:8, 10:7, 10:6, 10:5, 10:4, 10:3, 10:2, 10:1, 1:99, 5:95, 10:90, 15:85, 20:80, 25:75, 30:70, 35:65, 40:60, 45:55, 50:50, 55:45, 60:40, 65:35, 70:30, 75:25, 80:20, 85:15, 90:10, 95:5, or 99:1 in vitro or in vivo. In some embodiments, the guide to passenger ratio refers to the ratio of the guide strands to the passenger strands after the intracellular processing of the pri-microRNA. For example, a 80:20 guide-to-passenger ratio would have 8 guide strands to every 2 passenger strands processed from the precursor.

In some embodiments, the passenger to guide (P: G) (also referred to as the sense to antisense) strand ratio is 1:10, 1:9, 1:8, 1:7, 1:6, 1:5, 1:4, 1:3, 1:2, 1;1, 2:10, 2:9, 2:8, 2:7, 2:6, 2:5, 2:4, 2:3, 2:2, 2:1, 3:10, 3:9, 3:8, 3:7, 3:6, 3:5, 3:4, 3:3, 3:2, 3:1, 4:10, 4:9, 4:8, 4:7, 4:6, 4:5, 4:4, 4:3, 4:2, 4:1, 5:10, 5:9, 5:8, 5:7, 5:6, 5:5, 5:4, 5:3, 5:2, 5:1, 6:10, 6:9, 6:8, 6:7, 6:6, 6:5, 6:4, 6:3, 6:2, 6:1, 7:10, 7:9, 7:8, 7:7, 7:6, 7:5, 7:4, 7:3, 7:2, 7:1, 8:10, 8:9, 8:8, 8:7, 8:6, 8:5, 8:4, 8:3, 8:2, 8:1, 9:10, 9:9, 9:8, 9:7, 9:6, 9:5, 9:4, 9:3, 9:2, 9:1, 10:10, 10:9, 10:8, 10:7, 10:6, 10:5, 10:4, 10:3, 10:2, 10:1, 1:99, 5:95, 10:90, 15:85, 20:80, 25:75, 30:70, 35:65, 40:60, 45:55, 50:50, 55:45, 60:40, 65:35, 70:30, 75:25, 80:20, 85:15, 90:10, 95:5, or 99:1 in vitro or in vivo. In some embodiments, the passenger to guide ratio refers to the ratio of the passenger strands to the guide strands after the excision of the guide strand. For example, an 80:20 passenger to guide ratio would have 8 passenger strands to every 2 guide strands processed from the precursor.

In certain embodiments, the siRNA molecules may be used to silence wild type or mutant version of the gene of interest by targeting at least one exon on the gene of interest sequence. The exon may be exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, exon 29, exon 30, exon 31, exon 32, exon 33, exon 34, exon 35, exon 36, exon 37, exon 38, exon 39, exon 40, exon 41, exon 42, exon 43, exon 44, exon 45, exon 46, exon 47, exon 48, exon 49, exon 50, exon 51, exon 52, exon 53, exon 54, exon 55, exon 56, exon 57, exon 58, exon 59, exon 60, exon 61, exon 62, exon 63, exon 64, exon 65, exon 66, and/or exon 67.

### Molecular Scaffold

In some embodiments, a viral genome described herein encodes a modulatory polynucleotide comprising a siRNA, e.g., an siRNA described herein for targeting SOD1. In some embodiments, a viral genome described herein comprises a modulatory polynucleotide encoding a siRNA, e.g., an siRNA described herein for targeting SOD1. In some embodiments, the modulatory polynucleotide comprises a molecular scaffold. In some embodiments, a molecular scaffold comprises a framework or starting molecule that forms the sequence or structural basis against which to design or make a subsequent molecule. In some embodiments, the modulatory polynucleotide comprises a 5′ flanking region, a loop region, and a 3′ flanking region. In some embodiments, the modulatory polynucleotide further comprises a passenger strand (e.g., a passenger strand or guide strand sequence of a siRNA for targeting SOD1 described herein) and/or a guide strand (e.g., a guide strand or antisense strand sequence of a siRNA for targeting SOD1 described herein). In some embodiments, the modulatory polynucleotide is an RNA molecule, e.g., an encoded modulatory polynucleotide. In some embodiments, the modulatory polynucleotide is a DNA molecule, e.g., a DNA molecule encoding the RNA modulatory polynucleotide.

In some embodiments, the encoded modulatory polynucleotide comprises in 5′ to 3′ order: a 5′ flanking region, a passenger strand (e.g., a passenger strand or guide strand sequence of a siRNA for targeting SOD1 described herein), a loop region, a guide strand (e.g., a guide strand or antisense strand sequence of a siRNA for targeting SOD1 described herein), and a 3′ flanking region. In some embodiments, the encoded modulatory polynucleotide is an RNA molecule.

In some embodiments, the modulatory polynucleotide comprises in 5′ to 3′ order: a 5′ flanking region, a guide strand (e.g., a guide strand or antisense strand sequence of a siRNA for targeting SOD1 described herein), a loop region, a passenger strand (e.g., a passenger strand or sense strand sequence of a siRNA for targeting SOD1 described herein), and a 3′ flanking region. In some embodiments, the modulatory polynucleotide is a DNA molecule.

In some embodiments, the modulatory polynucleotide comprises a stem-loop structure. In some embodiments, the passenger strand and the guide strand are located, respectively, on a 5′ arm and a 3′ arm of a stem loop structure, wherein the passenger strand is located between the 5′ flanking region and the loop region and the guide strand is located between the loop region and the 3′ flanking region. In some embodiments, the guide strand and the passenger strand are located, respectively, on a 5′ arm and a 3′ arm of a stem loop structure, wherein the guide strand is located between the 5′ flanking region and the loop region and the passenger strand is located between the loop region and the 3′ flanking region.

In some embodiments the 5′ and 3′ flanking sequences comprise the same nuncleotide sequence. In some embodiments, the 5′ and 3′ flanking regions comprise different nucleotide sequences. In some embodiments, the loop sequence comprises a nucleic acid sequence encoding at least one UGUG motif. In some embodiments, the nucleic acid sequence encoding the UGUG motif is located at the 5′ terminus of the loop sequence.

In some embodiments, a molecular scaffold described herein comprises the nucleotide sequence of a 5′ flanking region, a loop region and a 3′ flanking region, provided in Tables 11-13, or a fragment thereof. In some embodiments, the 5′ flanking region, loop region, and 3′ flanking region comprises or is encoded by a nucleotide sequence provided in Tables 11-13, or a fragment thereof.

TABLE 11
Exemplary 5′ Flanking Regions
for Molecular Scaffold

| | | SEQ |
|---|---|---|
| Name | Sequence | ID NO |
| 5F1 (DNA) | CTCCCGCAGAACACCATGCGCTCCACGGAA | 2547 |
| 5F1 (RNA) | CUCCCGCAGAACACCAUGCGCUCCACGGAA | 5000 |
| 5F2 (DNA) | GAAGCAAAGAAGGGGCAGAGGGAGCCCGTG | 2548 |
| | AGCTGAGTGGGCCAGGGACTGGGAGAAGGA | |
| | GTGAGGAGGCAGGGCCGGCATGCCTCTGCT | |
| | GCTGGCCAGA | |
| 5F2 (RNA) | GAAGCAAAGAAGGGGCAGAGGGAGCCCGUG | 5001 |
| | AGCUGAGUGGGCCAGGGACUGGGAGAAGGA | |
| | GUGAGGAGGCAGGGCCGGCAUGCCUCUGCU | |
| | GCUGGCCAGA | |
| 5F3 (DNA) | GTGCTGGGCGGGGGGCGGCGGGCCCTCCCG | 2549 |
| | CAGAACACCATGCGCTCTTCGGAA | |
| 5F3 (RNA) | GUGCUGGGCGGGGGGCGGCGGGCCCUCCCG | 5002 |
| | CAGAACACCAUGCGCUCUUCGGAA | |
| 5F4 (DNA) | GTGCTGGGCGGGGGGCGGCGGGCCCTCCCG | 5014 |
| | CAGAACACCATGCGCTCCACGGAA | |
| 5F4 (RNA) | GUGCUGGGCGGGGGGCGGCGGGCCCUCCCG | 5003 |
| | CAGAACACCAUGCGCUCCACGGAA | |

TABLE 12
Exemplary Loop Regions for Molecular Scaffold

| Name | Sequence | SEQ ID NO |
|---|---|---|
| L1 (DNA) | GTGGCCACTGAGAAG | 2550 |
| L1 (RNA) | GUGGCCACUGAGAAG | 5004 |
| L2 (DNA) | GTCTGCACCTGTCACTAG | 2551 |
| L2 (RNA) | GUCUGCACCUGUCACUAG | 5005 |
| L3 (DNA) | TGTGACCTGG | 2552 |
| L3 (RNA) | UGUGACCUGG | 5006 |
| L4 (DNA) | TGTGATTTGG | 2553 |
| LA (RNA) | UGUGAUUUGG | 5007 |

TABLE 13
Exemplary 3′ Flanking Regions
for Molecular Scaffold

| | | SEQ |
|---|---|---|
| Name | Sequence | ID NO |
| 3F1 (DNA) | CTGAGGAGCGCCTTGACAGCAGCCATGGGAGG | 2554 |
| | GCC | |
| 3F1 (RNA) | CUGAGGAGCGCCUUGACAGCAGCCAUGGGAGG | 5008 |
| | GCC | |
| 3F2 (DNA) | TGGCCGTGTAGTGCTACCCAGCGCTGGCTGCC | 2555 |
| | TCCTCAGCATTGCAATTCCTCTCCCATCTGGG | |
| | CACCAGTCAGCTACCCTGGTGGGAATCTGGGT | |
| | AGCC | |
| 3F2(RNA) | UGGCCGUGUAGUGCUACCCAGCGCUGGCUGCC | 5009 |
| | UCCUCAGCAUUGCAAUUCCUCUCCCAUCUGGG | |
| | CACCAGUCAGCUACCCUGGUGGGAAUCUGGGU | |
| | AGCC | |
| 3F3 (DNA) | GGCCGTGTAGTGCTACCCAGCGCTGGCTGCCT | 2556 |
| | CCTCAGCATTGCAATTCCTCTCCCATCTGGGC | |
| | ACCAGTCAGCTACCCTGGTGGGAATCTGGGTA | |
| | GCC | |
| 3F3 (RNA) | GGCCGUGUAGUGCUACCCAGCGCUGGCUGCCU | 5010 |
| | CCUCAGCAUUGCAAUUCCUCUCCCAUCUGGGC | |
| | ACCAGUCAGCUACCCUGGUGGGAAUCUGGGUA | |
| | GCC | |
| 3F4 (DNA) | CTGAGGAGCGCCTTGACAGCAGCCATGGGAGG | 2557 |
| | GCCGCCCCCTACCTCAGTGA | |
| 3F4 (RNA) | CUGAGGAGCGCCUUGACAGCAGCCAUGGGAGG | 5011 |
| | GCCGCCCCCUACCUCAGUGA | |
| 3F5 (DNA) | CTGTGGAGCGCCTTGACAGCAGCCATGGGAGG | 2558 |
| | GCCGCCCCCTACCTCAGTGA | |
| 3F5 (RNA) | CUGUGGAGCGCCUUGACAGCAGCCAUGGGAGG | 5012 |
| | GCCGCCCCCUACCUCAGUGA | |

Any of the regions described in Table 11, Table 12, and Table 13, or a fragment of variant thereof may be used in the molecular scaffolds described herein, e.g., a molecular scaffold encoding or comprising a siRNA for targeting SOD1. It is understood that in some embodiments, any of the sequences in Tables 11-13 herein, can comprise a U nucleotide (uridine) in place of any of or all T nucleotides (thymine) within the sequence (e.g., an RNA molecule). It is understood that in some embodiments, any of the sequences in Tables 11-13 herein, can comprise a T nucleotide (thymine) in place of any of or all U nucleotides (uridine) within the sequence (e.g., an DNA molecule).

In some embodiments, the modulatory polynucleotide comprises a 5′ flanking region, fragment or variant thereof, e.g., as listed in Table 11 or as provided in WO2016077689, WO2017201248, WO2018204797, WO2017201258, WO2018204803, or WO2021016505, the contents of which are hereby incorporated by reference in their entirety.

In some embodiments, the 5′ flanking region comprises the nucleotide sequence of any of SEQ ID NOs: 2547-2549, or 5014, a nucleotide sequence, substantially identical (e.g., having at least 70%, 75%, 80%, 85%, 90%, 95% 96%, 97%, 98%, 99% sequence identity) thereto; a nucleotide sequence comprising one, two, three, four, five, six, or seven, but no more than ten different nucleotides relative to the nucleotide sequence of any of SEQ ID NOs: 2547-2549, or 5014; or a nucleotide sequence comprising one, two, three, four, five, six, or seven, but no more than ten modifications relative to the nucleotide sequence of any of SEQ ID NOs: 2547-2549, or 5014.

In some embodiments, the encoded 5′ flanking region comprises the nucleotide sequence of any one of SEQ ID NOs: 5000-5003; a nucleotide sequence substantially identical (e.g., having at least 70%, 75%, 80%, 85%, 90%, 95% 96%, 97%, 98%, 99% sequence identity) thereto; a nucleotide sequence comprising one, two, three, four, five, six, or seven, but no more than ten different nucleotides relative to the nucleotide sequence of any of SEQ ID NOs: 5000-5003; or a nucleotide sequence comprising one, two, three, four, five, six, or seven, but no more than ten modifications relative to the nucleotide sequence of any of SEQ ID NOs: 5000-5003.

In some embodiments, the nucleotide sequence encoding the 5′ flanking region comprises the nucleotide sequence of any of SEQ ID NOs: 2547-2549, or 5014, a nucleotide sequence, substantially identical (e.g., having at least 70%, 75%, 80%, 85%, 90%, 95% 96%, 97%, 98%, 99% sequence identity) thereto; a nucleotide sequence comprising one, two, three, four, five, six, or seven, but no more than ten different nucleotides relative to the nucleotide sequence of any of SEQ ID NOs: 2547-2549, or 5014; or a nucleotide sequence comprising one, two, three, four, five, six, or seven, but no more than ten modifications relative to the nucleotide sequence of any of SEQ ID NOs: 2547-2549, or 5014.

In some embodiments, the 5′ flanking region comprises the nucleotide sequence of SEQ ID NO: 2547; a nucleotide sequence, substantially identical (e.g., having at least 70%, 75%, 80%, 85%, 90%, 95% 96%, 97%, 98%, 99% sequence identity) to SEQ ID NO: 2547; a nucleotide sequence comprising one, two, three, four, five, six, or seven, but no more than ten different nucleotides relative to the nucleotide sequence of SEQ ID NO: 2547; or a nucleotide sequence comprising one, two, three, four, five, six, or seven, but no more than ten modifications relative to the nucleotide sequence of SEQ ID NO: 2547.

In some embodiments, the encoded 5′ flanking region comprises the nucleotide sequence of SEQ ID NO: 5000; a nucleotide sequence, substantially identical (e.g., having at least 70%, 75%, 80%, 85%, 90%, 95% 96%, 97%, 98%, 99% sequence identity) to SEQ ID NO: 5000; a nucleotide sequence comprising one, two, three, four, five, six, or seven, but no more than ten different nucleotides relative to the nucleotide sequence of SEQ ID NO: 5000; or a nucleotide sequence comprising one, two, three, four, five, six, or seven, but no more than ten modifications relative to the nucleotide sequence of SEQ ID NO: 5000. In some embodiments, the nucleotide sequence encoding the 5′ flanking region comprises the nucleotide sequence of SEQ ID NO: 2547; a nucleotide sequence, substantially identical (e.g., having at least 70%, 75%, 80%, 85%, 90%, 95% 96%, 97%, 98%, 99% sequence identity) to SEQ ID NO: 2547; a nucleotide sequence comprising one, two, three, four, five, six, or seven, but no more than ten different nucleotides relative to the nucleotide sequence of SEQ ID NO: 2547; or a nucleotide sequence comprising one, two, three, four, five, six, or seven, but no more than ten modifications relative to the nucleotide sequence of SEQ ID NO: 2547.

In some embodiments, the encoded 5′ flanking region comprises the nucleotide sequence of SEQ ID NO: 5001; a nucleotide sequence, substantially identical (e.g., having at least 70%, 75%, 80%, 85%, 90%, 95% 96%, 97%, 98%, 99% sequence identity) to SEQ ID NO: 5001; a nucleotide sequence comprising one, two, three, four, five, six, or seven, but no more than ten different nucleotides relative to the nucleotide sequence of SEQ ID NO: 5001; or a nucleotide sequence comprising one, two, three, four, five, six, or seven, but no more than ten modifications relative to the nucleotide sequence of SEQ ID NO: 5001. In some embodiments, the nucleotide sequence encoding the 5′ flanking region comprises the nucleotide sequence of SEQ ID NO: 2548; a nucleotide sequence, substantially identical (e.g., having at least 70%, 75%, 80%, 85%, 90%, 95% 96%, 97%, 98%, 99% sequence identity) to SEQ ID NO: 2548; a nucleotide sequence comprising one, two, three, four, five, six, or seven, but no more than ten different nucleotides relative to the nucleotide sequence of SEQ ID NO: 2548; or a nucleotide sequence comprising one, two, three, four, five, six, or seven, but no more than ten modifications relative to the nucleotide sequence of SEQ ID NO: 2548.

In some embodiments, the modulatory polynucleotide comprises a loop region, fragment or variant thereof, e.g., as provided in Table 12 or as provided in WO2016077689, WO2017201248, WO2018204797, WO2017201258, WO2018204803, or WO2021016505, the contents of which are hereby incorporated by reference in their entirety.

In some embodiments, the loop region comprises the nucleotide sequence of any of SEQ ID NOs: 2550-2553; a nucleotide sequence substantially identical (e.g., having at least 70%, 75%, 80%, 85%, 90%, 95% 96%, 97%, 98%, 99% sequence identity) thereto; a nucleotide sequence comprising one, two, three, or four, but no more than five different nucleotides relative to the nucleotide sequence of any of SEQ ID NOs: 2550-2553; or a nucleotide sequence comprising one, two, three, or four, but no more than five modifications relative to the nucleotide sequence of any of SEQ ID NOs: 2550-2553.

In some embodiments, the encoded loop region comprises the nucleotide sequence of any of SEQ ID NOs: 5004-5007; a nucleotide sequence substantially identical (e.g., having at least 70%, 75%, 80%, 85%, 90%, 95% 96%, 97%, 98%, 99% sequence identity) thereto; a nucleotide sequence comprising one, two, three, or four, but no more than five different nucleotides relative to the nucleotide sequence of any of SEQ ID NOs: 5004-5007; or a nucleotide sequence comprising one, two, three, or four, but no more than five modifications relative to the nucleotide sequence of any of SEQ ID NOs: 5004-5007.

In some embodiments, the nucleotide sequence encoding the loop region comprises the nucleotide sequence of any of SEQ ID NOs: 2550-2553; a nucleotide sequence substantially identical (e.g., having at least 70%, 75%, 80%, 85%, 90%, 95% 96%, 97%, 98%, 99% sequence identity) thereto; a nucleotide sequence comprising one, two, three, or four, but no more than five different nucleotides relative to the nucleotide sequence of any of SEQ ID NOs: 2550-2553; or a nucleotide sequence comprising one, two, three, or four, but no more than five modifications relative to the nucleotide sequence of any of SEQ ID NOs: 2550-2553.

In some embodiments, the loop region comprises the nucleotide sequence of SEQ ID NO: 2550; a nucleotide sequence substantially identical (e.g., having at least 70%, 75%, 80%, 85%, 90%, 95% 96%, 97%, 98%, 99% sequence identity) to SEQ ID NO: 2550; a nucleotide sequence comprising one, two, three, or four, but no more than five different nucleotides relative to the nucleotide sequence of 2550; or a nucleotide sequence comprising one, two, three, or four, but no more than five modifications relative to the nucleotide sequence of SEQ ID NO: 2550.

In some embodiments, the encoded loop region comprises the nucleotide sequence of 5004; a nucleotide sequence substantially identical (e.g., having at least 70%, 75%, 80%, 85%, 90%, 95% 96%, 97%, 98%, 99% sequence identity) to SEQ ID NO: 5004; a nucleotide sequence comprising one, two, three, or four, but no more than five different nucleotides relative to the nucleotide sequence of 4887; or a nucleotide sequence comprising one, two, three, or four, but no more than five modifications relative to the nucleotide sequence of SEQ ID NO: 5004. In some embodiments, the nucleotide sequence encoding the loop region comprises the nucleotide sequence of SEQ ID NO: 2550; a nucleotide sequence substantially identical (e.g., having at least 70%, 75%, 80%, 85%, 90%, 95% 96%, 97%, 98%, 99% sequence identity) to SEQ ID NO: 2550; a nucleotide sequence comprising one, two, three, or four, but no more than five different nucleotides relative to the nucleotide sequence of 4363; or a nucleotide sequence comprising one, two, three, or four, but no more than five modifications relative to the nucleotide sequence of SEQ ID NO: 2550.

In some embodiments, the encoded loop region comprises the nucleotide sequence of 5005; a nucleotide sequence substantially identical (e.g., having at least 70%, 75%, 80%, 85%, 90%, 95% 96%, 97%, 98%, 99% sequence identity) to SEQ ID NO: 5005; a nucleotide sequence comprising one, two, three, or four, but no more than five different nucleotides relative to the nucleotide sequence of 4887; or a nucleotide sequence comprising one, two, three, or four, but no more than five modifications relative to the nucleotide sequence of SEQ ID NO: 5005. In some embodiments, the nucleotide sequence encoding the loop region comprises the nucleotide sequence of SEQ ID NO: 2551; a nucleotide sequence substantially identical (e.g., having at least 70%, 75%, 80%, 85%, 90%, 95% 96%, 97%, 98%, 99% sequence identity) to SEQ ID NO: 2551; a nucleotide sequence comprising one, two, three, or four, but no more than five different nucleotides relative to the nucleotide sequence of 4363; or a nucleotide sequence comprising one, two, three, or four, but no more than five modifications relative to the nucleotide sequence of SEQ ID NO: 2551.

In some embodiments, the modulatory polynucleotide comprises a 3′ flanking region, fragment or variant thereof, e.g., as listed in Table 13 or as provided in WO2016077689, WO2017201248, WO2018204797, WO2017201258, WO2018204803, or WO2021016505, the contents of which are hereby incorporated by reference in their entirety.

In some embodiments, the 3′ flanking region comprises the nucleotide sequence of any of SEQ ID NOs: 2554-2558; a nucleotide sequence, substantially identical (e.g., having at least 70%, 75%, 80%, 85%, 90%, 95% 96%, 97%, 98%, 99% sequence identity) thereto; a nucleotide sequence comprising one, two, three, four, five, six, or seven, but no more than ten different nucleotides relative to the nucleotide sequence of any of SEQ ID NOs: 2554-2558; or a nucleotide sequence comprising one, two, three, four, five, six, or seven, but no more than ten modifications relative to the nucleotide sequence of any of SEQ ID NOs: 2554-2558.

In some embodiments, the encoded 3′ flanking region comprises the nucleotide sequence of any one of SEQ ID NOs: 5008-5012; a nucleotide sequence substantially identical (e.g., having at least 70%, 75%, 80%, 85%, 90%, 95% 96%, 97%, 98%, 99% sequence identity) thereto; a nucleotide sequence comprising one, two, three, four, five, six, or seven, but no more than ten modifications relative to the nucleotide sequence of any of SEQ ID NOs: 5008-5012; or a nucleotide sequence comprising one, two, three, four, five, six, or seven, but no more than ten modifications relative to the nucleotide sequence of any of SEQ ID NOs: 5008-5012.

In some embodiments, the nucleotide sequence encoding the 3′ flanking region comprises the nucleotide sequence of any of SEQ ID NOs: 2554-2558; a nucleotide sequence, substantially identical (e.g., having at least 70%, 75%, 80%, 85%, 90%, 95% 96%, 97%, 98%, 99% sequence identity) thereto; a nucleotide sequence comprising one, two, three, four, five, six, or seven, but no more than ten different nucleotides relative to the nucleotide sequence of any of SEQ ID NOs: 2554-2558; or a nucleotide sequence comprising one, two, three, four, five, six, or seven, but no more than ten modifications relative to the nucleotide sequence of any of SEQ ID NOs: 2554-2558.

In some embodiments, the 3′ flanking region comprises the nucleotide sequence of SEQ ID NO: 2554; a nucleotide sequence, substantially identical (e.g., having at least 70%, 75%, 80%, 85%, 90%, 95% 96%, 97%, 98%, 99% sequence identity) to SEQ ID NO: 2554; a nucleotide sequence comprising one, two, three, four, five, six, or seven, but no more than ten modifications relative to the nucleotide sequence of SEQ ID NO: 2554; or a nucleotide sequence comprising one, two, three, four, five, six, or seven, but no more than ten modifications relative to the nucleotide sequence of SEQ ID NO: 2554.

In some embodiments, the encoded 3′ flanking region comprises the nucleotide sequence of SEQ ID NO: 5008; a nucleotide sequence substantially identical (e.g., having at least 70%, 75%, 80%, 85%, 90%, 95% 96%, 97%, 98%, 99% sequence identity) to SEQ ID NO: 5008; a nucleotide sequence comprising one, two, three, four, five, six, or seven, but no more than ten different nucleotides relative to the nucleotide sequence of 5008; or a nucleotide sequence comprising one, two, three, four, five, six, or seven, but no more than ten modifications relative to the nucleotide sequence of SEQ ID NO: 5008. In some embodiments, the nucleotide sequence encoding the 3′ flanking region comprises the nucleotide sequence of 2554; a nucleotide sequence, substantially identical (e.g., having at least 70%, 75%, 80%, 85%, 90%, 95% 96%, 97%, 98%, 99% sequence identity) to SEQ ID NO: 2554; a nucleotide sequence comprising one, two, three, four, five, six, or seven, but no more than ten different nucleotides relative to the nucleotide sequence of SEQ ID NO: 2554; or a nucleotide sequence comprising one, two, three, four, five, six, or seven, but no more than ten modifications relative to the nucleotide sequence of SEQ ID NO: 2554.

In some embodiments, the encoded 3′ flanking region comprises the nucleotide sequence of SEQ ID NO: 5009; a nucleotide sequence substantially identical (e.g., having at least 70%, 75%, 80%, 85%, 90%, 95% 96%, 97%, 98%, 99% sequence identity) to SEQ ID NO: 5009; a nucleotide sequence comprising one, two, three, four, five, six, or seven, but no more than ten different nucleotides relative to the nucleotide sequence of 5009; or a nucleotide sequence comprising one, two, three, four, five, six, or seven, but no more than ten modifications relative to the nucleotide sequence of SEQ ID NO: 5009. In some embodiments, the nucleotide sequence encoding the 3′ flanking region comprises the nucleotide sequence of 2555; a nucleotide sequence, substantially identical (e.g., having at least 70%, 75%, 80%, 85%, 90%, 95% 96%, 97%, 98%, 99% sequence identity) to SEQ ID NO: 2555; a nucleotide sequence comprising one, two, three, four, five, six, or seven, but no more than ten different nucleotides relative to the nucleotide sequence of SEQ ID NO: 2555; or a nucleotide sequence comprising one, two, three, four, five, six, or seven, but no more than ten modifications relative to the nucleotide sequence of SEQ ID NO: 2555.

In some embodiments, the encoded modulatory polynucleotide comprises: (i) a 5′ flanking region comprising the nucleotide sequence of any one of SEQ ID NOs: 5000-5003, a nucleotide sequence substantially identical (e.g., having at least 70%, 75%, 80%, 85%, 90%, 95% 96%, 97%, 98%, 99% sequence identity) thereto, a nucleotide sequence comprising one, two, three, four, five, six, or seven, but no more than ten different nucleotides relative to the nucleotide sequence of any one of SEQ ID NOs: 5000-5003; or a nucleotide sequence comprising one, two, three, four, five, six, or seven, but no more than ten different nucleotides relative to the nucleotide sequence of any one of SEQ ID NOs: 5000-5003; (ii) a loop region comprising the nucleotide sequence of any one of SEQ ID NOs: 5004-5007, a nucleotide sequence substantially identical (e.g., having at least 70%, 75%, 80%, 85%, 90%, 95% 96%, 97%, 98%, 99% sequence identity) thereto, a nucleotide sequence comprising one, two, three, or four, but no more than five different nucleotides relative to the nucleotide sequence of any one of SEQ ID NOs: 5004-5007; or a nucleotide sequence comprising one, two, three, or four, but no more than five different nucleotides relative to the nucleotide sequence of any one of SEQ ID NOs: 5004-5007; and (iii) a 3′ flanking region comprising the nucleotide sequence of any one of SEQ ID NOs: 5008-5012, a nucleotide sequence substantially identical (e.g., having at least 70%, 75%, 80%, 85%, 90%, 95% 96%, 97%, 98%, 99% sequence identity) thereto, a nucleotide sequence comprising one, two, three, four, five, six, or seven, but no more than ten different nucleotides relative to the nucleotide sequence of any one of SEQ ID NOs: 5008-5012, a nucleotide sequence comprising one, two, three, four, five, six, or seven, but no more than ten modifications relative to the nucleotide sequence of any one of SEQ ID NOs: 5008-5012.

In some embodiments, the modulatory polynucleotide comprises: (i) a 5′ flanking region comprising the nucleotide sequence of any one of SEQ ID NOs: 2547-2548 or 5014, a nucleotide sequence substantially identical (e.g., having at least 70%, 75%, 80%, 85%, 90%, 95% 96%, 97%, 98%, 99% sequence identity) thereto, a nucleotide sequence comprising one, two, three, four, five, six, or seven, but no more than ten different nucleotides relative to the nucleotide sequence of any one of SEQ ID NOs: 2547-2548 or 5014; or a nucleotide sequence comprising one, two, three, four, five, six, or seven, but no more than ten different nucleotides relative to the nucleotide sequence of any one of SEQ ID NOs: 2547-2548 or 5014; (ii) a loop region comprising the nucleotide sequence of any one of SEQ ID NOs: 2550-2553, a nucleotide sequence substantially identical (e.g., having at least 70%, 75%, 80%, 85%, 90%, 95% 96%, 97%, 98%, 99% sequence identity) thereto, a nucleotide sequence comprising one, two, three, or four, but no more than five different nucleotides relative to the nucleotide sequence of any one of SEQ ID NOs: 2550-2553; or a nucleotide sequence comprising one, two, three, or four, but no more than five different nucleotides relative to the nucleotide sequence of any one of SEQ ID NOs: 2550-2553; and (iii) a 3′ flanking region comprising the nucleotide sequence of any one of SEQ ID NOs: 2554-2558, a nucleotide sequence substantially identical (e.g., having at least 70%, 75%, 80%, 85%, 90%, 95% 96%, 97%, 98%, 99% sequence identity) thereto, a nucleotide sequence comprising one, two, three, four, five, six, or seven, but no more than ten different nucleotides relative to the nucleotide sequence of any one of SEQ ID NOs: 2554-2558, a nucleotide sequence comprising one, two, three, four, five, six, or seven, but no more than ten modifications relative to the nucleotide sequence of any one of SEQ ID NOs: 2554-2558.

In some embodiments, the encoded modulatory polynucleotide comprises: (i) a 5′ flanking region comprising the nucleotide sequence of SEQ ID NO: 5000, a nucleotide sequence substantially identical (e.g., having at least 70%, 75%, 80%, 85%, 90%, 95% 96%, 97%, 98%, 99% sequence identity) to SEQ ID NO: 5000, a nucleotide sequence comprising one, two, three, four, five, six, or seven, but no more than ten different nucleotides relative to the nucleotide sequence of SEQ ID NO: 5000; or a nucleotide sequence comprising one, two, three, four, five, six, or seven, but no more than ten different nucleotides relative to the nucleotide sequence of SEQ ID NO: 5000; (ii) a loop region comprising the nucleotide sequence of SEQ ID NO: 5004, a nucleotide sequence substantially identical (e.g., having at least 70%, 75%, 80%, 85%, 90%, 95% 96%, 97%, 98%, 99% sequence identity) to SEQ ID NO: 5004, a nucleotide sequence comprising one, two, three, or four, but no more than five different nucleotides relative to the nucleotide sequence of SEQ ID NO: 5004; or a nucleotide sequence comprising one, two, three, or four, but no more than five different nucleotides relative to the nucleotide sequence of SEQ ID NO: 5004; and (iii) a 3′ flanking region comprising the nucleotide sequence of SEQ ID NO: 5008, a nucleotide sequence substantially identical (e.g., having at least 70%, 75%, 80%, 85%, 90%, 95% 96%, 97%, 98%, 99% sequence identity) to SEQ ID NO: 5008, a nucleotide sequence comprising one, two, three, four, five, six, or seven, but no more than ten different nucleotides relative to the nucleotide sequence of SEQ ID NO: 5008, a nucleotide sequence comprising one, two, three, four, five, six, or seven, but no more than ten modifications relative to the nucleotide sequence of SEQ ID NO: 5008.

In some embodiments, the modulatory polynucleotide comprises: (i) a 5′ flanking region comprising the nucleotide sequence of SEQ ID NO: 2547, a nucleotide sequence substantially identical (e.g., having at least 70%, 75%, 80%, 85%, 90%, 95% 96%, 97%, 98%, 99% sequence identity) to SEQ ID NO: 2547, a nucleotide sequence comprising one, two, three, four, five, six, or seven, but no more than ten different nucleotides relative to the nucleotide sequence of SEQ ID NO: 2547; or a nucleotide sequence comprising one, two, three, four, five, six, or seven, but no more than ten different nucleotides relative to the nucleotide sequence of SEQ ID NO: 2547; (ii) a loop region comprising the nucleotide sequence of SEQ ID NO: 2550, a nucleotide sequence substantially identical (e.g., having at least 70%, 75%, 80%, 85%, 90%, 95% 96%, 97%, 98%, 99% sequence identity) to SEQ ID NO: 2550, a nucleotide sequence comprising one, two, three, or four, but no more than five different nucleotides relative to the nucleotide sequence of SEQ ID NO: 2550; or a nucleotide sequence comprising one, two, three, or four, but no more than five different nucleotides relative to the nucleotide sequence of SEQ ID NO: 2550; and (iii) a 3′ flanking region comprising the nucleotide sequence of SEQ ID NO: 2554, a nucleotide sequence substantially identical (e.g., having at least 70%, 75%, 80%, 85%, 90%, 95% 96%, 97%, 98%, 99% sequence identity) to SEQ ID NO: 2554, a nucleotide sequence comprising one, two, three, four, five, six, or seven, but no more than ten different nucleotides relative to the nucleotide sequence of SEQ ID NO: 2554, a nucleotide sequence comprising one, two, three, four, five, six, or seven, but no more than ten modifications relative to the nucleotide sequence of SEQ ID NO: 2554.

In some embodiments, the encoded modulatory polynucleotide comprises: (i) a 5′ flanking region comprising the nucleotide sequence of SEQ ID NO: 5001, a nucleotide sequence substantially identical (e.g., having at least 70%, 75%, 80%, 85%, 90%, 95% 96%, 97%, 98%, 99% sequence identity) to SEQ ID NO: 5001, a nucleotide sequence comprising one, two, three, four, five, six, or seven, but no more than ten different nucleotides relative to the nucleotide sequence of SEQ ID NO: 5001; or a nucleotide sequence comprising one, two, three, four, five, six, or seven, but no more than ten different nucleotides relative to the nucleotide sequence of SEQ ID NO: 5001; (ii) a loop region comprising the nucleotide sequence of SEQ ID NO: 5005, a nucleotide sequence substantially identical (e.g., having at least 70%, 75%, 80%, 85%, 90%, 95% 96%, 97%, 98%, 99% sequence identity) to SEQ ID NO: 5005, a nucleotide sequence comprising one, two, three, or four, but no more than five different nucleotides relative to the nucleotide sequence of SEQ ID NO: 5005; or a nucleotide sequence comprising one, two, three, or four, but no more than five different nucleotides relative to the nucleotide sequence of SEQ ID NO: 5005; and (iii) a 3′ flanking region comprising the nucleotide sequence of SEQ ID NO: 5009, a nucleotide sequence substantially identical (e.g., having at least 70%, 75%, 80%, 85%, 90%, 95% 96%, 97%, 98%, 99% sequence identity) to SEQ ID NO: 5009, a nucleotide sequence comprising one, two, three, four, five, six, or seven, but no more than ten different nucleotides relative to the nucleotide sequence of SEQ ID NO: 5009, a nucleotide sequence comprising one, two, three, four, five, six, or seven, but no more than ten modifications relative to the nucleotide sequence of SEQ ID NO: 5009.

In some embodiments, the modulatory polynucleotide comprises: (i) a 5′ flanking region comprising the nucleotide sequence of SEQ ID NO: 2548, a nucleotide sequence substantially identical (e.g., having at least 70%, 75%, 80%, 85%, 90%, 95% 96%, 97%, 98%, 99% sequence identity) to SEQ ID NO: 2548, a nucleotide sequence comprising one, two, three, four, five, six, or seven, but no more than ten different nucleotides relative to the nucleotide sequence of SEQ ID NO: 2548; or a nucleotide sequence comprising one, two, three, four, five, six, or seven, but no more than ten different nucleotides relative to the nucleotide sequence of SEQ ID NO: 2548; (ii) a loop region comprising the nucleotide sequence of SEQ ID NO: 2551, a nucleotide sequence substantially identical (e.g., having at least 70%, 75%, 80%, 85%, 90%, 95% 96%, 97%, 98%, 99% sequence identity) to SEQ ID NO: 2551, a nucleotide sequence comprising one, two, three, or four, but no more than five different nucleotides relative to the nucleotide sequence of SEQ ID NO: 2551; or a nucleotide sequence comprising one, two, three, or four, but no more than five different nucleotides relative to the nucleotide sequence of SEQ ID NO: 2551; and (iii) a 3′ flanking region comprising the nucleotide sequence of SEQ ID NO: 2555, a nucleotide sequence substantially identical (e.g., having at least 70%, 75%, 80%, 85%, 90%, 95% 96%, 97%, 98%, 99% sequence identity) to SEQ ID NO: 2555, a nucleotide sequence comprising one, two, three, four, five, six, or seven, but no more than ten different nucleotides relative to the nucleotide sequence of SEQ ID NO: 2555, a nucleotide sequence comprising one, two, three, four, five, six, or seven, but no more than ten modifications relative to the nucleotide sequence of SEQ ID NO: 2555.

In certain embodiments, the molecular scaffold may be a natural pri-miRNA scaffold. As a non-limiting example, the molecular scaffold may be a scaffold derived from the human miR155 scaffold.

In certain embodiments, the molecular scaffold may comprise one or more linkers known in the art. The linkers may separate regions or one molecular scaffold from another. As a non-limiting example, the molecular scaffold may be polycistronic.

Modulatory Polynucleotide Comprising Molecular Scaffold and siRNA Molecules Targeting SOD1

In some embodiments, the modulatory polynucleotide comprises 5′ and 3′ flanking regions, loop region, and nucleic acid sequences encoding sense sequence and antisense sequence as described in Table 14.

In some embodiments, the modulatory polynucleotide comprises the nucleotide sequence of any one of SEQ ID NOs: 2562, 2579, 5022, 2559, 2560, 2561, 2563-2578, or nucleotide sequence at least 70%, 75%, 80%, 85%, 90%, 95%, or 99% identical thereto. In some embodiments, the modulatory polynucleotide comprises a nucleotide sequence comprising at least one, two, three, four, five, six, or seven, but no more than ten different nucleotides relative to the nucleotide sequence of any one of SEQ ID NOs: 2562, 2579, 5022, 2559, 2560, 2561, or 2563-2578. In some embodiments, the modulatory polynucleotide comprises a nucleotide sequence comprising at least one, two, three, four, five, six, or seven, but no more than ten modifications relative to the nucleotide sequence of any one of SEQ ID NOs: 2562, 2579, 5022, 2559, 2560, 2561, or 2563-2578.

In some embodiments, the modulatory polynucleotide comprises the nucleotide sequence of SEQ ID NOs: 2562; a nucleotide sequence at least 70%, 75%, 80%, 85%, 90%, 95%, or 99% identical to SEQ ID NO: 2562; a nucleotide sequence comprising at least one, two, three, four, five, six, or seven, but no more than ten different nucleotides relative to the nucleotide sequence of SEQ ID NO: 2562; or a nucleotide sequence comprising at least one, two, three, four, five, six, or seven, but no more than ten modifications relative to the nucleotide sequence of SEQ ID NO: 2562.

In some embodiments, the encoded modulatory polynucleotide comprises the nucleotide sequence of SEQ ID NO: 5013; a nucleotide sequence at least 70%, 75%, 80%, 85%, 90%, 95%, or 99% identical to SEQ ID NO: 5013; a nucleotide sequence comprising at least one, two, three, four, five, six, or seven, but no more than ten different nucleotides relative to the nucleotide sequence of SEQ ID NO: 5013; or a nucleotide sequence comprising at least one, two, three, four, five, six, or seven, but no more than ten modifications relative to the nucleotide sequence of SEQ ID NO: 5013.

It is understood that in some embodiments, any of the sequences in Table 14 herein, can comprise a U nucleotide (uridine) in place of any of or all T nucleotides (thymine) within the sequence (e.g., an RNA molecule). It is understood that in some embodiments, any of the sequences in Table 14 herein, can comprise a T nucleotide (thymine) in place of any of or all U nucleotides (uridine) within the sequence (e.g., a DNA molecule).

TABLE 14
SOD1 Modulatory Polynucleotide Sequence Regions (5′ to 3′)

| Modulatory | | | |
|---|---|---|---|
| Polynucleotide | SEQ | | |
| Construct Name | ID NO | Description | Sequence |
| VOYSOD1miR10 | 2562 | 5′ Flanking to | CTCCCGCAGAACACCATGCGCTCCACGGAAGCAGGTCCTCA |
| 4-788.2 | | 3′ Flanking | CTTTAATGCTGTGGCCACTGAGAAGTATTAAAGTGAGGACC |
| | | (DNA) | TGCTTCTGAGGAGCGCCTTGACAGCAGCCATGGGAGGGCC |
| | 5013 | 5′ Flanking to | CUCCCGCAGAACACCAUGCGCUCCACGGAAGCAGGUCCUCA |
| | | 3′ Flanking | CUUUAAUGCUGUGGCCACUGAGAAGUAUUAAAGUGAGGACC |
| | | (RNA) | UGCUUCUGAGGAGCGCCUUGACAGCAGCCAUGGGAGGGCC |
| | 2547 | 5′ Flanking | CTCCCGCAGAACACCATGCGCTCCACGGAA |
| | 66 | Passenger | GCAGGTCCTCACTTTAATGCT |
| | 2550 | Loop | GTGGCCACTGAGAAG |
| | 1351 | Guide | TATTAAAGTGAGGACCTGCTT |
| | 2554 | 3′Flanking | CTGAGGAGCGCCTTGACAGCAGCCATGGGAGGGCC |
| VOYSOD1miR12 | 2579 | 5′ Flanking to | GAAGCAAAGAAGGGGCAGAGGGAGCCCGTGAGCTGAGTGGG |
| 7-860 | | 3′ Flanking | CCAGGGACTGGGAGAAGGAGTGAGGAGGCAGGGCCGGCATG |
| | | | CCTCTGCTGCTGGCCAGACCCCTTAACTCATTTGTTCCCGT |
| | | | CTGCACCTGTCACTAGTAACAGATGAGTTAAGGGGTTTGGC |
| | | | CGTGTAGTGCTACCCAGCGCTGGCTGCCTCCTCAGCATTGC |
| | | | AATTCCTCTCCCATCTGGGCACCAGTCAGCTACCCTGGTGG |
| | | | GAATCTGGGTAGCC |
| | 4821 | 5′ Flanking to | GAAGCAAAGAAGGGGCAGAGGGAGCCCGUGAGCUGAGUGGG |
| | | 3′ Flanking | CCAGGGACUGGGAGAAGGAGUGAGGAGGCAGGGCCGGCAUG |
| | | | CCUCUGCUGCUGGCCAGACCCCUUAACUCAUUUGUUCCCGU |
| | | | CUGCACCUGUCACUAGUAACAGAUGAGUUAAGGGGUUUGGC |
| | | | CGUGUAGUGCUACCCAGCGCUGGCUGCCUCCUCAGCAUUGC |
| | | | AAUUCCUCUCCCAUCUGGGCACCAGUCAGCUACCCUGGUGG |
| | | | GAAUCUGGGUAGCC |
| | 2548 | 5` Flanking | GAAGCAAAGAAGGGGCAGAGGGAGCCCGTGAGCTGAGTGGG |
| | | | CCAGGGACTGGGAGAAGGAGTGAGGAGGCAGGGCCGGCATG |
| | | | CCTCTGCTGCTGGCCAGA |
| | 1347 | Passenger | CCCCTTAACTCATTTGTTCCC |
| | 2551 | Loop | GTCTGCACCTGTCACTAG |
| | 1368 | Guide | TAACAGATGAGTTAAGGGGTT |
| | 2555 | 3′Flanking | TGGCCGTGTAGTGCTACCCAGCGCTGGCTGCCTCCTCAGCA |
| | | | TTGCAATTCCTCTCCCATCTGGGCACCAGTCAGCTACCCTG |
| | | | GTGGGAATCTGGGTAGCC |
| VOYSOD1miR11 | 5022 | 5′ Flanking to | GTGCTGGGCGGGGGGCGGCGGGCCCTCCCGCAGAACACCAT |
| 4-861 | | 3′ Flanking | GCGCTCTTCGGAACCCTTAACTGATCTGTTAACCTGTGACC |
| | | | TGGTTAACAGATGAGTTAAGGGTTCTGTGGAGCGCCTTGAC |
| | | | AGCAGCCATGGGAGGGCCGCCCCCTACCTCAGTGA |
| | 2549 | 5′ Flanking | GTGCTGGGCGGGGGGCGGCGGGCCCTCCCGCAGAACACCAT |
| | | | GCGCTCTTCGGAA |
| | 5023 | Passenger | CCCTTAACTGATCTGTTAACC |
| | 2552 | Loop | TGTGACCTGG |
| | 5024 | Guide | TTAACAGATGAGTTAAGGGTT |
| | 2558 | 3′Flanking | CTGTGGAGCGCCTTGACAGCAGCCATGGGAGGGCCGCCCCC |
| | | | TACCTCAGTGA |
| VOYSOD1miR10 | 2559 | 5′ Flanking to | GTGCTGGGCGGGGGGCGGCGGGCCCTCCCGCAGAACACCAT |
| 2-788 | | 3′ Flanking | GCGCTCTTCGGAAGCAGGTCCTCACTTTAATGCCTGTGACC |
| | | | TGGGATTAAAGTGAGGACCTGCTTCTGAGGAGCGCCTTGAC |
| | | | AGCAGCCATGGGAGGGCCGCCCCCTACCTCAGTGA |
| | 2549 | 5′ Flanking | GTGCTGGGCGGGGGGCGGCGGGCCCTCCCGCAGAACACCAT |
| | | | GCGCTCTTCGGAA |
| | 1328 | Passenger | GCAGGTCCTCACTTTAATGCC |
| | 2552 | Loop | TGTGACCTGG |
| | 1348 | Guide | GATTAAAGTGAGGACCTGCTT |
| | 2557 | 3′Flanking | CTGAGGAGCGCCTTGACAGCAGCCATGGGAGGGCCGCCCCC |
| | | | TACCTCAGTGA |
| VOYSOD1miR10 | 2560 | 5′ Flanking to | GTGCTGGGCGGGGGGCGGCGGGCCCTCCCGCAGAACACCAT |
| 2-805c | | 3′ Flanking | GCGCTCTTCGGAAGGCAATGTGACTGCTGGCCCCTGTGACC |
| | | | TGGTGCCAGCAGTCACATTGCCTTCTGAGGAGCGCCTTGAC |
| | | | AGCAGCCATGGGAGGGCCGCCCCCTACCTCAGTGA |
| | 2549 | 5′ Flanking | GTGCTGGGCGGGGGGCGGCGGGCCCTCCCGCAGAACACCAT |
| | | | GCGCTCTTCGGAA |
| | 1329 | Passenger | GGCAATGTGACTGCTGGCCCC |
| | 2552 | Loop | TGTGACCTGG |
| | 1349 | Guide | TGCCAGCAGTCACATTGCCTT |
| | 2557 | 3′Flanking | CTGAGGAGCGCCTTGACAGCAGCCATGGGAGGGCCGCCCCC |
| | | | TACCTCAGTGA |
| VOYSOD1miR10 | 2561 | 5′ Flanking to | GTGCTGGGCGGGGGGCGGCGGGCCCTCCCGCAGAACACCAT |
| 4-788 | | 3′ Flanking | GCGCTCTTCGGAAGCAGGTCCTCACTTTAATTCCTGTGACC |
| | | | TGGGATTAAAGTGAGGACCTGCTTCTGAGGAGCGCCTTGAC |
| | | | AGCAGCCATGGGAGGGCCGCCCCCTACCTCAGTGA |
| | 2549 | 5′ Flanking | GTGCTGGGCGGGGGGCGGCGGGCCCTCCCGCAGAACACCAT |
| | | | GCGCTCTTCGGAA |
| | 1330 | Passenger | GCAGGTCCTCACTTTAATTCC |
| | 2552 | Loop | TGTGACCTGG |
| | 1350 | Guide | GATTAAAGTGAGGACCTGCTT |
| | 2557 | 3′Flanking | CTGAGGAGCGCCTTGACAGCAGCCATGGGAGGGCCGCCCCC |
| | | | TACCTCAGTGA |
| VOYSOD1miR10 | 2563 | 5′ Flanking to | CTCCCGCAGAACACCATGCGCTCCACGGAACAGGTCCTCAC |
| 4-789 | | 3′ Flanking | TTTAATCGCTGTGGCCACTGAGAAGTGATTAAAGTGAGGAC |
| | | | CTGTTCTGAGGAGCGCCTTGACAGCAGCCATGGGAGGGCC |
| | 2547 | 5′ Flanking | CTCCCGCAGAACACCATGCGCTCCACGGAA |
| | 1331 | Passenger | CAGGTCCTCACTTTAATCGCT |
| | 2550 | Loop | GTGGCCACTGAGAAG |
| | 1352 | Guide | TGATTAAAGTGAGGACCTGTT |
| | 2554 | 3′Flanking | CTGAGGAGCGCCTTGACAGCAGCCATGGGAGGGCC |
| VOYSOD1miR10 | 2564 | 5′ Flanking to | GTGCTGGGCGGGGGGCGGCGGGCCCTCCCGCAGAACACCAT |
| 4-805c | | 3′ Flanking | GCGCTCTTCGGAAGGCAATGTGACTGCTGGTGCCTGTGACC |
| | | | TGGTGCCAGCAGTCACATTGCCTTCTGAGGAGCGCCTTGAC |
| | | | AGCAGCCATGGGAGGGCCGCCCCCTACCTCAGTGA |
| | 2549 | 5′ Flanking | GTGCTGGGCGGGGGGCGGCGGGCCCTCCCGCAGAACACCAT |
| | | | GCGCTCTTCGGAA |
| | 1332 | Passenger | GGCAATGTGACTGCTGGTGCC |
| | 2552 | Loop | TGTGACCTGG |
| | 1353 | Guide | TGCCAGCAGTCACATTGCCTT |
| | 2557 | 3′Flanking | CTGAGGAGCGCCTTGACAGCAGCCATGGGAGGGCCGCCCCC |
| | | | TACCTCAGTGA |
| VOYSOD1miR10 | 2565 | 5′ Flanking to | CTCCCGCAGAACACCATGCGCTCCACGGAAGTCGTTTGGCT |
| 4-829 | | 3′ Flanking | TGTGGTGGCTGTGGCCACTGAGAAGTCACCACAAGCCAAAC |
| | | | GACTTCTGAGGAGCGCCTTGACAGCAGCCATGGGAGGGCC |
| | 2547 | 5′ Flanking | CTCCCGCAGAACACCATGCGCTCCACGGAA |
| | 1333 | Passenger | GTCGTTTGGCTTGTGGTGGCT |
| | 2550 | Loop | GTGGCCACTGAGAAG |
| | 1354 | Guide | TCACCACAAGCCAAACGACTT |
| | 2554 | 3′Flanking | CTGAGGAGCGCCTTGACAGCAGCCATGGGAGGGCC |
| VOYSOD1miR10 | 2566 | 5′ Flanking to | CTCCCGCAGAACACCATGCGCTCCACGGAATCGTTTGGCTT |
| 4-830 | | 3′ Flanking | GTGGTGTGCTGTGGCCACTGAGAAGTACACCACAAGCCAAA |
| | | | CGATTCTGAGGAGCGCCTTGACAGCAGCCATGGGAGGGCC |
| | 2547 | 5′ Flanking | CTCCCGCAGAACACCATGCGCTCCACGGAA |
| | 1334 | Passenger | TCGTTTGGCTTGTGGTGTGCT |
| | 2550 | Loop | GTGGCCACTGAGAAG |
| | 1355 | Guide | TACACCACAAGCCAAACGATT |
| | 2554 | 3′ Flanking | CTGAGGAGCGCCTTGACAGCAGCCATGGGAGGGCC |
| VOYSOD1miR10 | 2567 | 5` Flanking to | GTGCTGGGCGGGGGGCGGCGGGCCCTCCCGCAGAACACCAT |
| 9-788 | | 3′ Flanking | GCGCTCTTCGGAAGCAGGTCCTCACTTTAATCCCTGTGATT |
| | | | TGGGATTAAAGTGAGGACCTGCTTCTGAGGAGCGCCTTGAC |
| | | | AGCAGCCATGGGAGGGCCGCCCCCTACCTCAGTGA |
| | 2549 | 5′ Flanking | GTGCTGGGCGGGGGGCGGCGGGCCCTCCCGCAGAACACCAT |
| | | | GCGCTCTTCGGAA |
| | 1335 | Passenger | GCAGGTCCTCACTTTAATCCC |
| | 2553 | Loop | TGTGATTTGG |
| | 1356 | Guide | GATTAAAGTGAGGACCTGCTT |
| | 2557 | 3′ Flanking | CTGAGGAGCGCCTTGACAGCAGCCATGGGAGGGCCGCCCCC |
| | | | TACCTCAGTGA |
| VOYSOD1miR10 | 2568 | 5′ Flanking to | GTGCTGGGCGGGGGGCGGCGGGCCCTCCCGCAGAACACCAT |
| 9-805c | | 3′ Flanking | GCGCTCTTCGGAAGGCAATGTGACTGCTGGTACCTGTGATT |
| | | | TGGTGCCAGCAGTCACATTGCCTTCTGAGGAGCGCCTTGAC |
| | | | AGCAGCCATGGGAGGGCCGCCCCCTACCTCAGTGA |
| | 2549 | 5′ Flanking | GTGCTGGGCGGGGGGCGGCGGGCCCTCCCGCAGAACACCAT |
| | | | GCGCTCTTCGGAA |
| | 1336 | Passenger | GGCAATGTGACTGCTGGTACC |
| | 2553 | Loop | TGTGATTTGG |
| | 1357 | Guide | TGCCAGCAGTCACATTGCCTT |
| | 2557 | 3′ Flanking | CTGAGGAGCGCCTTGACAGCAGCCATGGGAGGGCCGCCCCC |
| | | | TACCTCAGTGA |
| VOYSOD1miR11 | 2569 | 5′ Flanking to | GTGCTGGGGGGGGGCGGCGGGCCCTCCCGCAGAACACCAT |
| 4-788 | | 3′ Flanking | GCGCTCTTCGGAAGCAGGTCCTGACTTTAATCCCTGTGACC |
| | | | TGGGATTAAAGTGAGGACCTGCTTCTGTGGAGCGCCTTGAC |
| | | | AGCAGCCATGGGAGGGCCGCCCCCTACCTCAGTGA |
| | 2549 | 5′ Flanking | GTGCTGGGCGGGGGGCGGCGGGCCCTCCCGCAGAACACCAT |
| | | | GCGCTCTTCGGAA |
| | 1337 | Passenger | GCAGGTCCTGACTTTAATCCC |
| | 2552 | Loop | TGTGACCTGG |
| | 1358 | Guide | GATTAAAGTGAGGACCTGCTT |
| | 2558 | 3′Flanking | CTGTGGAGCGCCTTGACAGCAGCCATGGGAGGGCCGCCCCC |
| | | | TACCTCAGTGA |
| VOYSOD1miR11 | 2570 | 5` Flanking to | GTGCTGGGCGGGGGGCGGCGGGCCCTCCCGCAGAACACCAT |
| 4-805c | | 3′ Flanking | GCGCTCTTCGGAAGGCAATGTGTCTGCTGGTACCTGTGACC |
| | | | TGGTGCCAGCAGTCACATTGCCTTCTGTGGAGCGCCTTGAC |
| | | | AGCAGCCATGGGAGGGCCGCCCCCTACCTCAGTGA |
| | 2549 | 5′ Flanking | GTGCTGGGCGGGGGGCGGCGGGCCCTCCCGCAGAACACCAT |
| | | | GCGCTCTTCGGAA |
| | 1338 | Passenger | GGCAATGTGTCTGCTGGTACC |
| | 2552 | Loop | TGTGACCTGG |
| | 1359 | Guide | TGCCAGCAGTCACATTGCCTT |
| | 2558 | 3′Flanking | CTGTGGAGCGCCTTGACAGCAGCCATGGGAGGGCCGCCCCC |
| | | | TACCTCAGTGA |
| VOYSOD1miR11 | 2571 | 5′ Flanking to | GTGCTGGGCGGGGGGCGGCGGGCCCTCCCGCAGAACACCAT |
| 6-788 | | 3′ Flanking | GCGCTCTTCGGAAGCAGGTCCTCACTTTAATCCCTGTGACC |
| | | | TGGGATTAAAGTGAGGACCTGCTTCTGTGGAGCGCCTTGAC |
| | | | AGCAGCCATGGGAGGGCCGCCCCCTACCTCAGTGA |
| | 2549 | 5′ Flanking | GTGCTGGGCGGGGGGCGGCGGGCCCTCCCGCAGAACACCAT |
| | | | GCGCTCTTCGGAA |
| | 1339 | Passenger | GCAGGTCCTCACTTTAATCCC |
| | 2552 | Loop | TGTGACCTGG |
| | 1360 | Guide | GATTAAAGTGAGGACCTGCTT |
| | 2558 | 3′ Flanking | CTGTGGAGCGCCTTGACAGCAGCCATGGGAGGGCCGCCCCC |
| | | | TACCTCAGTGA |
| VOYSOD1miR11 | 2572 | 5′ Flanking to | GTGCTGGGCGGGGGGCGGCGGGCCCTCCCGCAGAACACCAT |
| 6-805c | | 3′ Flanking | GCGCTCTTCGGAAGGCAATGTGACTGCTGGTACCTGTGACC |
| | | | TGGTGCCAGCAGTCACATTGCCTTCTGTGGAGCGCCTTGAC |
| | | | AGCAGCCATGGGAGGGCCGCCCCCTACCTCAGTGA |
| | 2549 | 5′ Flanking | GTGCTGGGCGGGGGGCGGCGGGCCCTCCCGCAGAACACCAT |
| | | | GCGCTCTTCGGAA |
| | 1340 | Passenger | GGCAATGTGACTGCTGGTACC |
| | 2552 | Loop | TGTGACCTGG |
| | 1361 | Guide | TGCCAGCAGTCACATTGCCTT |
| | 2558 | 3′Flanking | CTGTGGAGCGCCTTGACAGCAGCCATGGGAGGGCCGCCCCC |
| | | | TACCTCAGTGA |
| VOYSOD1miR12 | 2573 | 5′ Flanking to | GAAGCAAAGAAGGGGCAGAGGGAGCCCGTGAGCTGAGTGGG |
| 7-788 | | 3′ Flanking | CCAGGGACTGGGAGAAGGAGTGAGGAGGCAGGGCCGGCATG |
| | | | CCTCTGCTGCTGGCCAGAGCAGGTCCTCACTTTAATCCCGT |
| | | | CTGCACCTGTCACTAGGATTAAAGTGAGGACCTGCTTTGGC |
| | | | CGTGTAGTGCTACCCAGCGCTGGCTGCCTCCTCAGCATTGC |
| | | | AATTCCTCTCCCATCTGGGCACCAGTCAGCTACCCTGGTGG |
| | | | GAATCTGGGTAGCC |
| | 2548 | 5′ Flanking | GAAGCAAAGAAGGGGCAGAGGGAGCCCGTGAGCTGAGTGGG |
| | | | CCAGGGACTGGGAGAAGGAGTGAGGAGGCAGGGCCGGCATG |
| | | | CCTCTGCTGCTGGCCAGA |
| | 1341 | Passenger | GCAGGTCCTCACTTTAATCCC |
| | 2551 | Loop | GTCTGCACCTGTCACTAG |
| | 1362 | Guide | GATTAAAGTGAGGACCTGCTT |
| | 2555 | 3′Flanking | TGGCCGTGTAGTGCTACCCAGCGCTGGCTGCCTCCTCAGCA |
| | | | TTGCAATTCCTCTCCCATCTGGGCACCAGTCAGCTACCCTG |
| | | | GTGGGAATCTGGGTAGCC |
| VOYSOD1miR12 | 2574 | 5′ Flanking to | GAAGCAAAGAAGGGGCAGAGGGAGCCCGTGAGCTGAGTGGG |
| 7-788.2 | | 3′ Flanking | CCAGGGACTGGGAGAAGGAGTGAGGAGGCAGGGCCGGCATG |
| | | | CCTCTGCTGCTGGCCAGAGCAGGTCCTCACTTTAATGCCGT |
| | | | CTGCACCTGTCACTAGTATTAAAGTGAGGACCTGCTTTGGC |
| | | | CGTGTAGTGCTACCCAGCGCTGGCTGCCTCCTCAGCATTGC |
| | | | AATTCCTCTCCCATCTGGGCACCAGTCAGCTACCCTGGTGG |
| | | | GAATCTGGGTAGCC |
| | 2548 | 5′ Flanking | GAAGCAAAGAAGGGGCAGAGGGAGCCCGTGAGCTGAGTGGG |
| | | | CCAGGGACTGGGAGAAGGAGTGAGGAGGCAGGGCCGGCATG |
| | | | CCTCTGCTGCTGGCCAGA |
| | 1342 | Passenger | GCAGGTCCTCACTTTAATGCC |
| | 2551 | Loop | GTCTGCACCTGTCACTAG |
| | 1363 | Guide | TATTAAAGTGAGGACCTGCTT |
| | 2555 | 3′ Flanking | TGGCCGTGTAGTGCTACCCAGCGCTGGCTGCCTCCTCAGCA |
| | | | TTGCAATTCCTCTCCCATCTGGGCACCAGTCAGCTACCCTG |
| | | | GTGGGAATCTGGGTAGCC |
| VOYSOD1miR12 | 2575 | 5′ Flanking to | GAAGCAAAGAAGGGGCAGAGGGAGCCCGTGAGCTGAGTGGG |
| 7-789 | | 3′ Flanking | CCAGGGACTGGGAGAAGGAGTGAGGAGGCAGGGCCGGCATG |
| | | | CCTCTGCTGCTGGCCAGACAGGTCCTCACTTTAATCGCCGT |
| | | | CTGCACCTGTCACTAGTGATTAAAGTGAGGACCTGTTTGGC |
| | | | CGTGTAGTGCTACCCAGCGCTGGCTGCCTCCTCAGCATTGC |
| | | | AATTCCTCTCCCATCTGGGCACCAGTCAGCTACCCTGGTGG |
| | | | GAATCTGGGTAGCC |
| | 2548 | 5′ Flanking | GAAGCAAAGAAGGGGCAGAGGGAGCCCGTGAGCTGAGTGGG |
| | | | CCAGGGACTGGGAGAAGGAGTGAGGAGGCAGGGCCGGCATG |
| | | | CCTCTGCTGCTGGCCAGA |
| | 1343 | Passenger | CAGGTCCTCACTTTAATCGCC |
| | 2551 | Loop | GTCTGCACCTGTCACTAG |
| | 1364 | Guide | TGATTAAAGTGAGGACCTGTT |
| | 2555 | 3′ Flanking | TGGCCGTGTAGTGCTACCCAGCGCTGGCTGCCTCCTCAGCA |
| | | | TTGCAATTCCTCTCCCATCTGGGCACCAGTCAGCTACCCTG |
| | | | GTGGGAATCTGGGTAGCC |
| VOYSOD1miR12 | 2576 | 5` Flanking to | GAAGCAAAGAAGGGGCAGAGGGAGCCCGTGAGCTGAGTGGG |
| 7-805c | | 3′ Flanking | CCAGGGACTGGGAGAAGGAGTGAGGAGGCAGGGCCGGCATG |
| | | | CCTCTGCTGCTGGCCAGAGGCAATGTGACTGCTGGTACCGT |
| | | | CTGCACCTGTCACTAGTGCCAGCAGTCACATTGCCTTTGGC |
| | | | CGTGTAGTGCTACCCAGCGCTGGCTGCCTCCTCAGCATTGC |
| | | | AATTCCTCTCCCATCTGGGCACCAGTCAGCTACCCTGGTGG |
| | | | GAATCTGGGTAGCC |
| | 2548 | 5′ Flanking | GAAGCAAAGAAGGGGCAGAGGGAGCCCGTGAGCTGAGTGGG |
| | | | CCAGGGACTGGGAGAAGGAGTGAGGAGGCAGGGCCGGCATG |
| | | | CCTCTGCTGCTGGCCAGA |
| | 1344 | Passenger | GGCAATGTGACTGCTGGTACC |
| | 2551 | Loop | GTCTGCACCTGTCACTAG |
| | 1365 | Guide | TGCCAGCAGTCACATTGCCTT |
| | 2555 | 3′Flanking | TGGCCGTGTAGTGCTACCCAGCGCTGGCTGCCTCCTCAGCA |
| | | | TTGCAATTCCTCTCCCATCTGGGCACCAGTCAGCTACCCTG |
| | | | GTGGGAATCTGGGTAGCC |
| VOYSOD1miR12 | 2577 | 5′ Flanking to | GAAGCAAAGAAGGGGCAGAGGGAGCCCGTGAGCTGAGTGGG |
| 7-829 | | 3′ Flanking | CCAGGGACTGGGAGAAGGAGTGAGGAGGCAGGGCCGGCATG |
| | | | CCTCTGCTGCTGGCCAGAGTCGTTTGGCTTGTGGTGGCCGT |
| | | | CTGCACCTGTCACTAGTCACCACAAGCCAAACGACTTTGGC |
| | | | CGTGTAGTGCTACCCAGCGCTGGCTGCCTCCTCAGCATTGC |
| | | | AATTCCTCTCCCATCTGGGCACCAGTCAGCTACCCTGGTGG |
| | | | GAATCTGGGTAGCC |
| | 2548 | 5′ Flanking | GAAGCAAAGAAGGGGCAGAGGGAGCCCGTGAGCTGAGTGGG |
| | | | CCAGGGACTGGGAGAAGGAGTGAGGAGGCAGGGCCGGCATG |
| | | | CCTCTGCTGCTGGCCAGA |
| | 1345 | Passenger | GTCGTTTGGCTTGTGGTGGCC |
| | 2551 | Loop | GTCTGCACCTGTCACTAG |
| | 1366 | Guide | TCACCACAAGCCAAACGACTTT |
| | 2556 | 3′Flanking | GGCCGTGTAGTGCTACCCAGCGCTGGCTGCCTCCTCAGCAT |
| | | | TGCAATTCCTCTCCCATCTGGGCACCAGTCAGCTACCCTGG |
| | | | TGGGAATCTGGGTAGCC |
| VOYSOD1miR12 | 2578 | 5′ Flanking to | GAAGCAAAGAAGGGGCAGAGGGAGCCCGTGAGCTGAGTGGG |
| 7-830 | | 3′ Flanking | CCAGGGACTGGGAGAAGGAGTGAGGAGGCAGGGCCGGCATG |
| | | | CCTCTGCTGCTGGCCAGATCGTTTGGCTTGTGGTGTGCCGT |
| | | | CTGCACCTGTCACTAGTACACCACAAGCCAAACGATTTGGC |
| | | | CGTGTAGTGCTACCCAGCGCTGGCTGCCTCCTCAGCATTGC |
| | | | AATTCCTCTCCCATCTGGGCACCAGTCAGCTACCCTGGTGG |
| | | | GAATCTGGGTAGCC |
| | 2548 | 5′ Flanking | GAAGCAAAGAAGGGGCAGAGGGAGCCCGTGAGCTGAGTGGG |
| | | | CCAGGGACTGGGAGAAGGAGTGAGGAGGCAGGGCCGGCATG |
| | | | CCTCTGCTGCTGGCCAGA |
| | 1346 | Passenger | TCGTTTGGCTTGTGGTGTGCC |
| | 2551 | Loop | GTCTGCACCTGTCACTAG |
| | 1367 | Guide | TACACCACAAGCCAAACGATT |
| | 2555 | 3′ Flanking | TGGCCGTGTAGTGCTACCCAGCGCTGGCTGCCTCCTCAGCA |
| | | | TTGCAATTCCTCTCCCATCTGGGCACCAGTCAGCTACCCTG |
| | | | GTGGGAATCTGGGTAGCC |

In some embodiments, the molecular scaffold may comprise a 5′ Flanking to 3′ Flanking Region sequence which comprises at least 20 (e.g., at least 21, 22, 23, 24, 25, 26, 27, 28, 29, or 30) contiguous nucleotides differing by no more than 5, 4, 3, 2 or 1 nucleotides from any one of the 5′ Flanking to 3′ Flanking Region sequences in Table 14. These differing nucleotides may be contiguous, or alternatively, they may be non-contiguous throughout the sequence. In some embodiments, the differing nucleotides are located at the ends of the sequences. In some embodiments, the nucleotide sequence encoding the siRNA duplex comprises the nucleotide sequence of any one of SEQ ID NOs: 2562, 2579, 5022, 2559, 2560, 2561, 2563, 2564, 2565, 2566, 2567, 2568, 2569, 2570, 2571, 2572, 2573, 2574, 2575, 2576, 2577, 2578 or a nucleotide sequence at least 70% (e.g., at least 75%, 80%, 85%, 90%, 92%, 95%, 97%, 98%, or 99%) identical thereto.

In some embodiments, the molecular scaffold may comprise a 5′ Flanking Region sequence which comprises at least 15 (e.g., at least 16, 17, 18, 19, 20, 21, 22, or 23) contiguous nucleotides differing by no more than 3, 2 or 1 nucleotides from any one of the 5′ Flanking Region sequences in Table 14. These differing nucleotides may be contiguous, or alternatively, they may be non-contiguous throughout the sequence. In some embodiments, the differing nucleotides are located at the ends of the sequences. In some embodiments, the 5′ Flanking Region includes a nucleotide from any one of the 5′ Flanking Region sequences in Table 14, or a nucleotide sequence substantially identical (e.g., having at least 70%, 75%, 80%, 85%, 90%, 92%, 95%, 97%, 98%, or 99% sequence identity) thereto.

In some embodiments, the molecular scaffold may comprise a passenger sequence which comprises at least 15 (e.g., at least 16, 17, 18, 19, 20, 21, 22, or 23) contiguous nucleotides differing by no more than 3, 2 or 1 nucleotides from any one of the passenger sequences in Table 14. These differing nucleotides may be contiguous, or alternatively, they may be non-contiguous throughout the sequence. In some embodiments, the differing nucleotides are located at the ends of the sequences.

In some embodiments, the passenger sequence includes a nucleotide from any one of the passenger sequences in Table 14, or a nucleotide sequence substantially identical (e.g., having at least 70%, 75%, 80%, 85%, 90%, 92%, 95%, 97%, 98%, or 99% sequence identity) thereto.

In some embodiments, the molecular scaffold may comprise a loop sequence which comprises at least 7 (e.g., at least 8, 9, or 10) contiguous nucleotides differing by no more than 3, 2 or 1 nucleotides from any one of the Loop sequences in Table 14. These differing nucleotides may be contiguous, or alternatively, they may be non-contiguous throughout the sequence. In some embodiments, the differing nucleotides are located at the ends of the sequences. In some embodiments, the loop sequence includes a nucleotide from any one of the loop sequences in Table 14, or a nucleotide sequence substantially identical (e.g., having at least 70%, 75%, 80%, 85%, 90%, 92%, 95%, 97%, 98%, or 99% sequence identity) thereto.

In some embodiments, the molecular scaffold may comprise a guide sequence which comprises at least 15 (e.g., at least 16, 17, 18, 19, 20, 21, 22, or 23) contiguous nucleotides differing by no more than 3, 2 or 1 nucleotides from any one of the guide sequences in Table 14. These differing nucleotides may be contiguous, or alternatively, they may be non-contiguous throughout the sequence. In some embodiments, the differing nucleotides are located at the ends of the sequences. In some embodiments, the guide sequence includes a nucleotide from any one of the guide sequences in Table 14, or a nucleotide sequence substantially identical (e.g., having at least 70%, 75%, 80%, 85%, 90%, 92%, 95%, 97%, 98%, or 99% sequence identity) thereto.

In some embodiments, the molecular scaffold may comprise a 3′ Flanking Region sequence which comprises at least 15 (e.g., at least 16, 17, 18, 19, 20, 21, 22, or 23) contiguous nucleotides differing by no more than 3, 2 or 1 nucleotides from any one of the 3′ Flanking Region sequences in Table 14. These differing nucleotides may be contiguous, or alternatively, they may be non-contiguous throughout the sequence. In some embodiments, the differing nucleotides are located at the ends of the sequences. In some embodiments, the 3′ Flanking Region sequence includes a nucleotide from any one of the 3′ Flanking Region sequences in Table 14, or a nucleotide sequence substantially identical (e.g., having at least 70%, 75%, 80%, 85%, 90%, 92%, 95%, 97%, 98%, or 99% sequence identity) thereto.

In some embodiments, the encoded modulatory polynucleotide comprises the nucleotide sequence of SEQ ID NO: 5013, or a nucleotide sequence at least 80%, 85%, 90%, 92%, 95%, 97%, 98%, or 99% identical thereto. In some embodiments, the nucleotide sequence encoding the modulatory polynucleotide comprises the nucleotide sequence of SEQ ID NO: 2562, or a nucleotide sequence at least 80%, 85%, 90%, 92%, 95%, 97%, 98%, or 99% identical thereto.

In some embodiments, the polynucleotide that targets SOD1 comprises the nucleotide sequence of SEQ ID NO: 2562, or a nucleotide sequence at least 80%, 85%, 90%, 92%, 95%, 97%, 98%, or 99% identical thereto. In some embodiments, the encoded polynucleotide that targets SOD1 comprises the nucleotide sequence of SEQ ID NO: 5013, or a nucleotide sequence at least 80%, 85%, 90%, 92%, 95%, 97%, 98%, or 99% identical thereto.

### AAV Particles Comprising Modulatory Polynucleotides

In certain embodiments, the AAV particle comprises a viral genome with a payload region comprising a modulatory polynucleotide sequence. In such an embodiment, a viral genome encoding more than one polypeptide may be replicated and packaged into a viral particle. A target cell transduced with a viral particle comprising a modulatory polynucleotide may express the encoded sense and/or antisense sequences in a single cell.

In some embodiments, the AAV particles are useful in the field of medicine for the treatment, prophylaxis, palliation or amelioration of neurological diseases and/or disorders.

In certain embodiments, the AAV particles comprising modulatory polynucleotide sequence which comprises a nucleic acid sequence encoding at least one siRNA molecule may be introduced into mammalian cells.

Where the AAV particle payload region comprises a modulatory polynucleotide, the modulatory polynucleotide may comprise sense and/or antisense sequences to knock down a target gene. The AAV viral genomes encoding modulatory polynucleotides described herein may be useful in the fields of human disease, viruses, infections veterinary applications and a variety of in vivo and in vitro settings.

In certain embodiments, the AAV particle viral genome may comprise at least one inverted terminal repeat (ITR) region. In some embodiments, the AAV particle viral genome may comprises one or more inverted terminal repeat (ITR) regions. In some embodiments, the AAV particle includes one ITR region. In some embodiments, the AAV particle includes two ITR sequence regions. As a non-limiting example, the viral genome comprises an ITR that is about 105 nucleotides in length and 141 nucleotides in length. As a non-limiting example, the viral genome comprises an ITR that is about 105 nucleotides in length and 130 nucleotides in length. As a non-limiting example, the viral genome comprises an ITR that is about 130 nucleotides in length and 141 nucleotides in length.

In certain embodiments, the AAV particle viral genome may comprise at least one filler sequence region.

In certain embodiments, the AAV particle viral genome may comprise at least one enhancer sequence region.

In certain embodiments, the AAV particle viral genome may comprise at least one exon sequence region.

In certain embodiments, the AAV particle viral genome may comprise at least one intron sequence region.

In certain embodiments, the AAV particle viral genome may comprise at least one polyadenylation signal sequence region.

In certain embodiments, the AAV particle viral genome comprises more than one polyA signal sequence region.

Additional ITR region nucleotides, filler region nucleotides, enhancer region nucleotides, exon and intron region nucleotides, and poly A signal sequence nucleotides that may be used in the nucleic acids encoding the SOD1 targeting polynucleotide described herein are disclosed in PCT Patent Application Publications WO2016077687, WO2019079240, WO2018204786, WO2019079242, WO 2020010042 and WO2020223296, which are hereby incorporated by reference in their entirety

Non-limiting examples of ITR to ITR sequences of AAV particles comprising a viral genome with a payload region comprising a modulatory polynucleotide sequence are described in Table 15. Table 15 provides ITR to ITR sequence of H1.mir104-788.2 with a lentivirus derived filler. Also provided in Table 15 are the components that are comprised within the ITR to ITR sequence. In some embodiments, the components may be separated from each other by vector backbone sequence. In some embodiments, the AAV viral genome comprises the nucleotide sequence of SEQ ID NO: 109, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 109. In some embodiments, the AAV viral genome comprises the nucleotide sequence of SEQ ID NO: 2562, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 2562.

In some embodiments, the AAV viral genome comprises the nucleotide sequence of SEQ ID NO: 109, or a nucleotide sequence at least 80%, 85%, 90%, 92%, 95%, 97%, 98%, or 99% identical thereto. In some embodiments, the AAV viral genome comprises the nucleotide sequence of SEQ ID NO: 109, or a nucleotide sequence at least 95% identical thereto. In some embodiments, the AAV viral genome comprises the nucleotide sequence of SEQ ID NO: 109, or a nucleotide sequence at least 98% identical thereto. In some embodiments, the AAV viral genome comprises the nucleotide sequence of SEQ ID NO: 109, or a nucleotide sequence at least 99% identical thereto. In some embodiments, the AAV viral genome comprises the nucleotide sequence of SEQ ID NO: 109.

TABLE 15

| | | | |
|---|---|---|---|
| ITR to ITR | | SEQ | |
| Construct Name | Region | ID NO | Sequence |

ITR to ITR Sequences of AAV Particles, H1.mir.104-788.2 (with lentivirus derived
filler) comprising a Modulatory Polynucleotide

| | | | |
|---|---|---|---|
| H1.mir.104- | ITR-ITR | 109 | CTGCGCGCTCGCTCGCTCACTGAGGCCGCCCGGGCAAAGCCCGGGC |
| 788.2 with | sequence | | GTCGGGCGACCTTTGGTCGCCCGGCCTCAGTGAGCGAGCGAGCGCG |
| lentivirus | | | CAGAGAGGGAGTGTAGCCATGCTCTAGGAAGATCAATTCAATTCAC |
| | | | GCGTCCATGGCTTAGAAGGCAAGAATCCTGGCTGTGGAAAGATACC |
| | | | TAAAGGATCAACAGCTCCTGGGGATTTGGGGTTGCTCTGGAAAACT |
| | | | CATTTGCACCACTGCTGTGCCTTGGAATGCTAGTTGGAGTAATAAA |
| | | | TCTCTGGAACAGATTTGGAATCACACGACCTGGATGGAGTGGGACA |
| | | | GAGAAATTAACAATTACACAAGCTTAATACACTCCTTAATTGAAGA |
| | | | ATCGCAAAACCAGCAAGAAAAGAATGAACAAGAATTATTGGAATTA |
| | | | GATAAATGGGCAAGTTTGTGGAATTGGTTTAACATAACAAATTGGC |
| | | | TGTGGTATATAAAATTATTCATAATGATAGTAGGAGGCTTGGTAGG |
| | | | TTTAAGAATAGTTTTTGCTGTACTTTCTATAGTGAATAGAGTTAGG |
| | | | CAGGGATATTCACCATTATCGTTTCAGACCCACCTCCCAACCCCGA |
| | | | GGGGACCCGACAGGCCCGAAGGAATAGAAGAAGAAGGTGGAGAGAG |
| | | | AGACAGAGACAGATCCATTCGATTAGTGAACGGATCTCGACGGTAT |
| | | | CGATCACGAGACTAGCCTCGAGCGGCCGCAATTCGAACGCTGACGT |
| | | | CATCAACCCGCTCCAAGGAATCGCGGGCCCAGTGTCACTAGGCGGG |
| | | | AACACCCAGCGCGCGTGCGCCCTGGCAGGAAGATGGCTGTGAGGGA |
| | | | CAGGGAGTGGCGCCCTGCAATATTTGCATGTCGCTATGTGTTCTGG |
| | | | GAAATCACCATAAACGTGAAATGTCTTTGGATTTGGGAATCTTATA |
| | | | AGTTCTGTATGAGACCACACCGGTACCGAGCTCTCCCGCAGAACAC |
| | | | CATGCGCTCCACGGAAGCAGGTCCTCACTTTAATGCTGTGGCCACT |
| | | | GAGAAGTATTAAAGTGAGGACCTGCTTCTGAGGAGCGCCTTGACAG |
| | | | CAGCCATGGGAGGGCCTCGAGGACGGGGTGAACTACGCCTGAGGAT |
| | | | CCGATCTTTTTCCCTCTGCCAAAAATTATGGGGACATCATGAAGCC |
| | | | CCTTGAGCATCTGACTTCTGGCTAATAAAGGAAATTTATTTTCATT |
| | | | GCAATAGTGTGTTGGAATTTTTTGTGTCTCTCACTCGGCCTAGGTA |
| | | | GATAAGTAGCATGGCGGGTTAATCATTAACTACAAGGAACCCCTAG |
| | | | TGATGGAGTTGGCCACTCCCTCTCTGCGCGCTCGCTCGCTCACTGA |
| | | | GGCCGGGCGACCAAAGGTCGCCCGACGCCCGGGCTTTGCCCGGGCG |
| | | | GCCTCAGTGAGCGAGCGAGCGCGCAG |
| derived filler | Modulatory | 2562 | CTCCCGCAGAACACCATGCGCTCCACGGAAGCAGGTCCTCACTTTA |
| (VOYSOD16) | Polynucleotide | | ATGCTGTGGCCACTGAGAAGTATTAAAGTGAGGACCTGCTTCTGAG |
| | | | GAGCGCCTTGACAGCAGCCATGGGAGGGCC |

ITR-ITR Components of H1.mir104-788.2 (with lentivirus derived filler)

| | | |
|---|---|---|
| 5 ′TTR | 126 | CTGCGCGCTCGCTCGCTCACTGAGGCCGCCCGGGCAAAGCCCGGGC |
| | | GTCGGGCGACCTTTGGTCGCCCGGCCTCAGTGAGCGAGCGAGCGCG |
| | | CAGAGAGGGAGTG |
| Lentivirus derived filler | 82 | CCATGGCTTAGAAGGCAAGAATCCTGGCTGTGGAAAGATACCTAAA |
| | | GGATCAACAGCTCCTGGGGATTTGGGGTTGCTCTGGAAAACTCATT |
| | | TGCACCACTGCTGTGCCTTGGAATGCTAGTTGGAGTAATAAATCTC |
| | | TGGAACAGATTTGGAATCACACGACCTGGATGGAGTGGGACAGAGA |
| | | AATTAACAATTACACAAGCTTAATACACTCCTTAATTGAAGAATCG |
| | | CAAAACCAGCAAGAAAAGAATGAACAAGAATTATTGGAATTAGATA |
| | | AATGGGCAAGTTTGTGGAATTGGTTTAACATAACAAATTGGCTGTG |
| | | GTATATAAAATTATTCATAATGATAGTAGGAGGCTTGGTAGGITTA |
| | | AGAATAGTTTTTGCTGTACTTTCTATAGTGAATAGAGTTAGGCAGG |
| | | GATATTCACCATTATCGTTTCAGACCCACCTCCCAACCCCGAGGGG |
| | | ACCCGACAGGCCCGAAGGAATAGAAGAAGAAGGTGGAGAGAGAGAC |
| | | AGAGACAGATCCATTCGATTAGTGAACGGATCTCGACGGTATCGAT |
| | | CACGAGACTAGCCTCGA |
| H1 promoter | 83 | GAACGCTGACGTCATCAACCCGCTCCAAGGAATCGCGGGCCCAGTG |
| | | TCACTAGGCGGGAACACCCAGCGCGCGTGCGCCCTGGCAGGAAGAT |
| | | GGCTGTGAGGGACAGGGAGTGGCGCCCTGCAATATTTGCATGTCGC |
| | | TATGTGTTCTGGGAAATCACCATAAACGTGAAATGTCTTTGGATTT |
| | | GGGAATCTTATAAGTTCTGTATGAGACCAC |
| Modulatory | 2562 | CTCCCGCAGAACACCATGCGCTCCACGGAAGCAGGTCCTCACTTTA |
| Polynucleotide | | ATGCTGTGGCCACTGAGAAGTATTAAAGTGAGGACCTGCTTCTGAG |
| (VOYSOD1-miR104-788.2) | | GAGCGCCTTGACAGCAGCCATGGGAGGGCC |
| rBGpA | 129 | GATCTTTTTCCCTCTGCCAAAAATTATGGGGACATCATGAAGCCCC |
| | | TTGAGCATCTGACTTCTGGCTAATAAAGGAAATTTATTTTCATTGC |
| | | AATAGTGTGTTGGAATTTTTTGTGTCTCTCACTCG |
| 3′ ITR | 130 | AGGAACCCCTAGTGATGGAGTTGGCCACTCCCTCTCTGCGCGCTCG |
| | | CTCGCTCACTGAGGCCGGGCGACCAAAGGTCGCCCGACGCCCGGGC |
| | | TTTGCCCGGGCGGCCTCAGTGAGCGAGCGAGCGCGCAG |

Table 16 provides ITR to ITR sequence of H1.mir104-788.2 with albumin derived filler. Also provided in Table 16 are the components that comprise the ITR to ITR sequence. In some embodiments, the components may be separated from each other by vector backbone sequence. In some embodiments, the AAV viral genome comprises the nucleotide sequence of SEQ ID NO: 125, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 125. In some embodiments, the AAV viral genome comprises the nucleotide sequence of SEQ ID NO: 2562, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 2562.

TABLE 16

| | | |
|---|---|---|
| | SEQ | |
| Description | ID NO | Sequence |

ITR to ITR of AAV Particles, H1.mir104-788.2 (with albumin derived filler)
comprising Modulatory Polynucleotides and its components

| | | |
|---|---|---|
| ITR to ITR of | 125 | CTGCGCGCTCGCTCGCTCACTGAGGCCGCCCGGGCAAAGCCCGGGCGTCGGGCGACC |
| H1.mir104- | | TTTGGTCGCCCGGCCTCAGTGAGCGAGCGAGCGCGCAGAGAGGGAGTGTAGCCATGC |
| 788.2 with | | TCTAGGAAGATCAATTCAATTCACGCGTATAGTCTTCTGCACAGGGCATTCTTTTTG |
| albumin derived | | CTTCAGGATGTTTACAACATTTGCTGCCCACTTTTCCTAGGTTTCTTGAGACCTCTA |
| filler | | CAAGAGTTGGAGTTGACACTTGGGGTACTTTCTTGGTGTAACGAACTAATAGCCTGA |
| | | AAAAAAGAAGTCATGTGTTTTCAGCAAGGCAAGAAACTGTCTAACATAGTAGATAAA |
| | | ACAGAGAACACTTGGCCGGAATCAACTAAGATGTTGCTATGTTCCATTCATCATATT |
| | | ATCTCCATCTGCAGAGTAGTGGGTTAGTGGAGGGTAGAAAACATTCTCCTGAACAAC |
| | | TAGTTAAACTTGGCTTTGAGTTCCACCTGTACCACTTGCATAATCTTGGGAAAGTGA |
| | | GTTGCCTAATTCAGTGACATTAATAAATTTATTAATTTCTTCTTTCAATAAAACCTG |
| | | GAGAGAGCTTCATATGTATCAGCATATGCTAAACTTGAAAGATACAAGTAGAAAATG |
| | | GAAGGAAATATATCTGACTCAATAGGGATAGTTCAAGGGTTAAATTAAAAGTAGTAA |
| | | AGTATTATAATTAATCTGACATGGTACCCTCTAGCGGCCGCAATTCGAACGCTGACG |
| | | TCATCAACCCGCTCCAAGGAATCGCGGGCCCAGTGTCACTAGGCGGGAACACCCAGC |
| | | GCGCGTGCGCCCTGGCAGGAAGATGGCTGTGAGGGACAGGGAGTGGCGCCCTGCAAT |
| | | ATTTGCATGTCGCTATGTGTTCTGGGAAATCACCATAAACGTGAAATGTCTTTGGAT |
| | | TTGGGAATCTTATAAGTTCTGTATGAGACCACACCGGTACCGAGCTCTCCCGCAGAA |
| | | CACCATGCGCTCCACGGAAGCAGGTCCTCACTTTAATGCTGTGGCCACTGAGAAGTA |
| | | TTAAAGTGAGGACCTGCTTCTGAGGAGCGCCTTGACAGCAGCCATGGGAGGGCCTCG |
| | | AGGACGGGGTGAACTACGCCTGAGGATCCGATCTTTTTCCCTCTGCCAAAAATTATG |
| | | GGGACATCATGAAGCCCCTTGAGCATCTGACTTCTGGCTAATAAAGGAAATTTATTT |
| | | TCATTGCAATAGTGTGTTGGAATTTTTTGTGTCTCTCACTCGGCCTAGGTAGATAAG |
| | | TAGCATGGCGGGTTAATCATTAACTACAAGGAACCCCTAGTGATGGAGTTGGCCACT |
| | | CCCTCTCTGCGCGCTCGCTCGCTCACTGAGGCCGGGCGACCAAAGGTCGCCCGACGC |
| | | CCGGGCTTTGCCCGGGCGGCCTCAGTGAGCGAGCGAGCGCGCAG |

ITR-ITR Components of H1.mir104-788.2 (with albumin derived filler)

| | | |
|---|---|---|
| 5′ITR | 126 | CTGCGCGCTCGCTCGCTCACTGAGGCCGCCCGGGCAAAGCCCGGGCGTCGGGCGACC |
| | | TTTGGTCGCCCGGCCTCAGTGAGCGAGCGAGCGCGCAGAGAGGGAGTG |
| Albumin | 127 | ATAGTCTTCTGCACAGGGCATTCTTTTTGCTTCAGGATGTTTACAACATTTGCTGCC |
| derived filler | | CACTTTTCCTAGGTTTCTTGAGACCTCTACAAGAGTTGGAGTTGACACTTGGGGTAC |
| | | TTTCTTGGTGTAACGAACTAATAGCCTGAAAAAAAGAAGTCATGTGTTTTCAGCAAG |
| | | GCAAGAAACTGTCTAACATAGTAGATAAAACAGAGAACACTTGGCCGGAATCAACTA |
| | | AGATGTTGCTATGTTCCATTCATCATATTATCTCCATCTGCAGAGTAGTGGGTTAGT |
| | | GGAGGGTAGAAAACATTCTCCTGAACAACTAGTTAAACTTGGCTTTGAGTTCCACCT |
| | | GTACCACTTGCATAATCTTGGGAAAGTGAGTTGCCTAATTCAGTGACATTAATAAAT |
| | | TTATTAATTTCTTCTTTCAATAAAACCTGGAGAGAGCTTCATATGTATCAGCATATG |
| | | CTAAACTTGAAAGATACAAGTAGAAAATGGAAGGAAATATATCTGACTCAATAGGGA |
| | | TAGTTCAAGGGTTAAATTAAAAGTAGTAAAGTATTATAATTAATCTGACATGGTACC |
| H1 promoter | 128 | AATTCGAACGCTGACGTCATCAACCCGCTCCAAGGAATCGCGGGCCCAGTGTCACTA |
| | | GGCGGGAACACCCAGCGCGCGTGCGCCCTGGCAGGAAGATGGCTGTGAGGGACAGGG |
| | | AGTGGCGCCCTGCAATATTTGCATGTCGCTATGTGTTCTGGGAAATCACCATAAACG |
| | | TGAAATGTCTTTGGATTTGGGAATCTTATAAGTTCTGTATGAGACCAC |
| Modulatory | 2562 | CTCCCGCAGAACACCATGCGCTCCACGGAAGCAGGTCCTCACTTTAATGCTGTGGCC |
| Polynucleotide | | ACTGAGAAGTATTAAAGTGAGGACCTGCTTCTGAGGAGCGCCTTGACAGCAGCCATG |
| (VOYSOD1- | | GGAGGGCC |
| miR104-788.2) | | |
| rBGpA | 129 | GATCTTTTTCCCTCTGCCAAAAATTATGGGGACATCATGAAGCCCCTTGAGCATCTG |
| | | ACTTCTGGCTAATAAAGGAAATTTATTTTCATTGCAATAGTGTGTTGGAATTTTTTG |
| | | TGTCTCTCACTCG |
| 3′ITR | 130 | AGGAACCCCTAGTGATGGAGTTGGCCACTCCCTCTCTGCGCGCTCGCTCGCTCACTG |
| | | AGGCCGGGCGACCAAAGGTCGCCCGACGCCCGGGCTTTGCCCGGGCGGCCTCAGTGA |
| | | GCGAGCGAGCGCGCAG |

Non-limiting examples of ITR to ITR sequences of AAV particles comprising a viral genome with a payload region comprising a modulatory polynucleotide sequence are described in Table 48. Table 48 provides ITR to ITR sequence of H1.mir127-860 with a lentivirus derived filler. Also provided in Table 48 are the components that are comprised within the ITR to ITR sequence. In some embodiments, the components may be separated from each other by backbone sequence. In some embodiments, the AAV viral genome comprises the nucleotide sequence of SEQ ID NO: 4820, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 4820. In some embodiments, the AAV viral genome comprises the nucleotide sequence of SEQ ID NO: 2579, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 2579.

TABLE 48

| | | | |
|---|---|---|---|
| ITR to ITR | | SEQ | |
| Construct Name | Region | ID NO | Sequence |

ITR to ITR Sequences of AAV Particles, H1.mir.127-860 (with lentivirus derived
filler) comprising a Modulatory Polynucleotide

| | | | |
|---|---|---|---|
| H1.mir.127- | ITR-ITR | 4820 | CTGCGCGCTCGCTCGCTCACTGAGGCCGCCCGGGCAAAGCCCGGGC |
| 860 with | sequence | | GTCGGGCGACCTTTGGTCGCCCGGCCTCAGTGAGCGAGCGAGCGCG |
| lentivirus | | | CAGAGAGGGAGTGTAGCCATGCTCTAGGAAGATCAATTCAATTCAC |
| | | | GCGTCCATGGCTTAGAAGGCAAGAATCCTGGCTGTGGAAAGATACC |
| | | | TAAAGGATCAACAGCTCCTGGGGATTTGGGGTTGCTCTGGAAAACT |
| | | | CATTTGCACCACTGCTGTGCCTTGGAATGCTAGTTGGAGTAATAAA |
| | | | TCTCTGGAACAGATTTGGAATCACACGACCTGGATGGAGTGGGACA |
| | | | GAGAAATTAACAATTACACAAGCTTAATACACTCCTTAATTGAAGA |
| | | | ATCGCAAAACCAGCAAGAAAAGAATGAACAAGAATTATTGGAATTA |
| | | | GATAAATGGGCAAGTTTGTGGAATTGGTTTAACATAACAAATTGGC |
| | | | TGTGGTATATAAAATTATTCATAATGATAGTAGGAGGCTTGGTAGG |
| | | | TTTAAGAATAGTTTTTGCTGTACTTTCTATAGTGAATAGAGTTAGG |
| | | | CAGGGATATTCACCATTATCGTTTCAGACCCACCTCCCAACCCCGA |
| | | | GGGGACCCGACAGGCCCGAAGGAATAGAAGAAGAAGGTGGAGAGAG |
| | | | AGACAGAGACAGATCCATTCGATTAGTGAACGGATCTCGACGGTAT |
| | | | CGATCACGAGACTAGCCTCGAGCGGCCGCAATTCGAACGCTGACGT |
| | | | CATCAACCCGCTCCAAGGAATCGCGGGCCCAGTGTCACTAGGCGGG |
| | | | AACACCCAGCGCGCGTGCGCCCTGGCAGGAAGATGGCTGTGAGGGA |
| | | | CAGGGAGTGGCGCCCTGCAATATTTGCATGTCGCTATGTGTTCTGG |
| | | | GAAATCACCATAAACGTGAAATGTCTTTGGATTTGGGAATCTTATA |
| | | | AGTTCTGTATGAGACCACACCGGTGAAGCAAAGAAGGGGCAGAGGG |
| | | | AGCCCGTGAGCTGAGTGGGCCAGGGACTGGGAGAAGGAGTGAGGAG |
| | | | GCAGGGCCGGCATGCCTCTGCTGCTGGCCAGACCCCTTAACTCATT |
| | | | TGTTCCCGTCTGCACCTGTCACTAGTAACAGATGAGTTAAGGGGTT |
| | | | TGGCCGTGTAGTGCTACCCAGCGCTGGCTGCCTCCTCAGCATTGCA |
| | | | ATTCCTCTCCCATCTGGGCACCAGTCAGCTACCCTGGTGGGAATCT |
| | | | GGGTAGCCCTCGAGGACGGGGTGAACTACGCCTGAGGATCCGATCT |
| | | | TTTTCCCTCTGCCAAAAATTATGGGGACATCATGAAGCCCCTTGAG |
| | | | CATCTGACTTCTGGCTAATAAAGGAAATTTATTTTCATTGCAATAG |
| | | | TGTGTTGGAATTTTTTGTGTCTCTCACTCGGCCTAGGTAGATAAGT |
| | | | AGCATGGCGGGTTAATCATTAACTACAAGGAACCCCTAGTGATGGA |
| | | | GTTGGCCACTCCCTCTCTGCGCGCTCGCTCGCTCACTGAGGCCGGG |
| | | | CGACCAAAGGTCGCCCGACGCCCGGGCTTTGCCCGGGCGGCCTCAG |
| | | | TGAGCGAGCGAGCGCGCAG |
| derived filler | Modulatory | 2579 | GAAGCAAAGAAGGGGCAGAGGGAGCCCGTGAGCTGAGTGGGCCAGG |
| (VOYSOD6) | Polynucleotide | | GACTGGGAGAAGGAGTGAGGAGGCAGGGCCGGCATGCCTCTGCTGC |
| | | | TGGCCAGACCCCTTAACTCATTTGTTCCCGTCTGCACCTGTCACTA |
| | | | GTAACAGATGAGTTAAGGGGTTTGGCCGTGTAGTGCTACCCAGCGC |
| | | | TGGCTGCCTCCTCAGCATTGCAATTCCTCTCCCATCTGGGCACCAG |
| | | | TCAGCTACCCTGGTGGGAATCTGGGTAGCC |

ITR-ITR Components of H1.mir127-860 (with lentivirus derived filler)

| | | |
|---|---|---|
| 5 ′TTR | 126 | CTGCGCGCTCGCTCGCTCACTGAGGCCGCCCGGGCAAAGCCCGGGC |
| | | GTCGGGCGACCTTTGGTCGCCCGGCCTCAGTGAGCGAGCGAGCGCG |
| | | CAGAGAGGGAGTG |
| Lentivirus derived filler | 82 | CCATGGCTTAGAAGGCAAGAATCCTGGCTGTGGAAAGATACCTAAA |
| | | GGATCAACAGCTCCTGGGGATTTGGGGTTGCTCTGGAAAACTCATT |
| | | TGCACCACTGCTGTGCCTTGGAATGCTAGTTGGAGTAATAAATCTC |
| | | TGGAACAGATTTGGAATCACACGACCTGGATGGAGTGGGACAGAGA |
| | | AATTAACAATTACACAAGCTTAATACACTCCTTAATTGAAGAATCG |
| | | CAAAACCAGCAAGAAAAGAATGAACAAGAATTATTGGAATTAGATA |
| | | AATGGGCAAGTTTGTGGAATTGGTTTAACATAACAAATTGGCTGTG |
| | | GTATATAAAATTATTCATAATGATAGTAGGAGGCTTGGTAGGTTTA |
| | | AGAATAGTTTTTGCTGTACTTTCTATAGTGAATAGAGTTAGGCAGG |
| | | GATATTCACCATTATCGTTTCAGACCCACCTCCCAACCCCGAGGGG |
| | | ACCCGACAGGCCCGAAGGAATAGAAGAAGAAGGTGGAGAGAGAGAC |
| | | AGAGACAGATCCATTCGATTAGTGAACGGATCTCGACGGTATCGAT |
| | | CACGAGACTAGCCTCGA |
| H1 promoter | 83 | GAACGCTGACGTCATCAACCCGCTCCAAGGAATCGCGGGCCCAGTG |
| | | TCACTAGGCGGGAACACCCAGCGCGCGTGCGCCCTGGCAGGAAGAT |
| | | GGCTGTGAGGGACAGGGAGTGGCGCCCTGCAATATTTGCATGTCGC |
| | | TATGTGTTCTGGGAAATCACCATAAACGTGAAATGTCTTTGGATTT |
| | | GGGAATCTTATAAGTTCTGTATGAGACCAC |
| Modulatory Polynucleotide | 2579 | GAAGCAAAGAAGGGGCAGAGGGAGCCCGTGAGCTGAGTGGGCCAGG |
| (VOYSOD1-miR127-860) | | GACTGGGAGAAGGAGTGAGGAGGCAGGGCCGGCATGCCTCTGCTGC |
| | | TGGCCAGACCCCTTAACTCATTTGTTCCCGTCTGCACCTGTCACTA |
| | | GTAACAGATGAGTTAAGGGGTTTGGCCGTGTAGTGCTACCCAGCGC |
| | | TGGCTGCCTCCTCAGCATTGCAATTCCTCTCCCATCTGGGCACCAG |
| | | TCAGCTACCCTGGTGGGAATCTGGGTAGCC |
| rBGpA | 129 | GATCTTTTTCCCTCTGCCAAAAATTATGGGGACATCATGAAGCCCC |
| | | TTGAGCATCTGACTTCTGGCTAATAAAGGAAATTTATTTTCATTGC |
| | | AATAGTGTGTTGGAATTTTTTGTGTCTCTCACTCG |
| 3′ ITR | 130 | AGGAACCCCTAGTGATGGAGTTGGCCACTCCCTCTCTGCGCGCTCG |
| | | CTCGCTCACTGAGGCCGGGCGACCAAAGGTCGCCCGACGCCCGGGC |
| | | TTTGCCCGGGCGGCCTCAGTGAGCGAGCGAGCGCGCAG |

Tables 17 and 18 provide further variant ITR to ITR sequences of mir104-788.2 with different promoters. In some embodiments, the components may be separated from each other by vector backbone sequence. In some embodiments, the AAV viral genome comprises a nucleotide sequence provided in Table 17 or 18, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications thereto.

TABLE 17
Additional ITR to ITR sequences of AAV Particles of mir104-788.2 comprising
Modulatory Polynucleotides and their components

| | | SEQ ID |
|---|---|---|
| Description | Sequence | NO |
| ITR to ITR of | CTGCGCGCTCGCTCGCTCACTGAGGCCGCCCGGGCAAAGCCCGGGCGTCGGGCGA | 4028 |
| CBA.mir104-788.2 | CCTTTGGTCGCCCGGCCTCAGTGAGCGAGCGAGCGCGCAGAGAGGGAGTGTAGCC | |
| with hβG | ATGCTCTAGGAAGATCAATTCAATTCACGCGTCGACATTGATTATTGACTAGTTA | |
| exon/intron/exon | TTAATAGTAATCAATTACGGGGTCATTAGTTCATAGCCCATATATGGAGTTCCGC | |
| (construct 1) | GTTACATAACTTACGGTAAATGGCCCGCCTGGCTGACCGCCCAACGACCCCCGCC | |
| | CATTGACGTCAATAATGACGTATGTTCCCATAGTAACGCCAATAGGGACTTTCCA | |
| | TTGACGTCAATGGGTGGAGTATTTACGGTAAACTGCCCACTTGGCAGTACATCAA | |
| | GTGTATCATATGCCAAGTACGCCCCCTATTGACGTCAATGACGGTAAATGGCCCG | |
| | CCTGGCATTATGCCCAGTACATGACCTTATGGGACTTTCCTACTTGGCAGTACAT | |
| | CTACGTATTAGTCATCGCTATTACCATGTCGAGGCCACGTTCTGCTTCACTCTCC | |
| | CCATCTCCCCCCCCTCCCCACCCCCAATTTTGTATTTATTTATTTTTTAATTATT | |
| | TTGTGCAGCGATGGGGGCGGGGGGGGGGGGCGCGCGCCAGGCGGGGCGGGGCGGG | |
| | GCGAGGGGCGGGGCGGGGCGAGGCGGAGAGGTGCGGCGGCAGCCAATCAGAGCGG | |
| | CGCGCTCCGAAAGTTTCCTTTTATGGCGAGGCGGCGGCGGCGGCGGCCCTATAAA | |
| | AAGCGAAGCGCGCGGCGGGCGGGAGCAAGCTTCGTTTAGTGAACCGTCAGATCGC | |
| | CTGGAGACGCCATCCACGCTGTTTTGACCTCCATAGAAGACACCGGGACCGATCC | |
| | AGCCTCCGCGGATTCGAATCCCGGCCGGGAACGGTGCATTGGAACGCGGATTCCC | |
| | CGTGCCAAGAGTGACGTAAGTACCGCCTATAGAGTCTATAGGCCCACAAAAAATG | |
| | CTTTCTTCTTTTAATATACTTTTTTGTTTATCTTATTTCTAATACTTTCCCTAAT | |
| | CTCTTTCTTTCAGGGCAATAATGATACAATGTATCATGCCTCTTTGCACCATTCT | |
| | AAAGAATAACAGTGATAATTTCTGGGTTAAGGCAATAGCAATATTTCTGCATATA | |
| | AATATTTCTGCATATAAATTGTAACTGATGTAAGAGGTTTCATATTGCTAATAGC | |
| | AGCTACAATCCAGCTACCATTCTGCTTTTATTTTATGGTTGGGATAAGGCTGGAT | |
| | TATTCTGAGTCCAAGCTAGGCCCTTTTGCTAATCATGTTCATACCTCTTATCTTC | |
| | CTCCCACAGCTCCTGGGCAACGTGCTGGTCTGTGTGCTGGCCCATCACTTTGGCA | |
| | AAGAATTGGGATTCGAACCGGTACCGAGCTCTCCCGCAGAACACCATGCGCTCCA | |
| | CGGAAGCAGGTCCTCACTTTAATGCTGTGGCCACTGAGAAGTATTAAAGTGAGGA | |
| | CCTGCTTCTGAGGAGCGCCTTGACAGCAGCCATGGGAGGGCCTCGAGGGGTGGCA | |
| | TCCCTGTGACCCCTCCCCAGTGCCTCTCCTGGCCCTGGAAGTTGCCACTCCAGTG | |
| | CCCACCAGCCTTGTCCTAATAAAATTAAGTTGCATCATTTTGTCTGACTAGGTGT | |
| | CCTTCTATAATATTATGGGGTGGAGGGGGGTGGTATGGAGCAAGGGGCAAGTTGG | |
| | GAAGACAACCTGTAGGGCCTGCGGGGTCTATTGGGAACCAAGCTGGAGTGCAGTG | |
| | GCACAATCTTGGCTCACTGCAATCTCCGCCTCCTGGGTTCAAGCGATTCTCCTGC | |
| | CTCAGCCTCCCGAGTTGTTGGGATTCCAGGCATGCATGACCAGGCTCAGCTAATT | |
| | TTTGTTTTTTTGGTAGAGACGGGGTTTCACCATATTGGCCAGGCTGGTCTCCAAC | |
| | TCCTAATCTCAGGTGATCTACCCACCTTGGCCTCCCAAATTGCTGGGATTACAGG | |
| | CGTGAACCACTGCTCCCTTCCCTGTCCTTATCGATGCCTAGGGTTAATCATTAAC | |
| | TACAAGGAACCCCTAGTGATGGAGTTGGCCACTCCCTCTCTGCGCGCTCGCTCGC | |
| | TCACTGAGGCCGGGCGACCAAAGGTCGCCCGACGCCCGGGCTTTGCCCGGGCGGC | |
| | CTCAGTGAGCGAGCGAGCGCGCAG | |
| ITR to ITR of | CTGCGCGCTCGCTCGCTCACTGAGGCCGCCCGGGCAAAGCCCGGGCGTCGGGCGA | 4029 |
| miniCBA.mir104- | CCTTTGGTCGCCCGGCCTCAGTGAGCGAGCGAGCGCGCAGAGAGGGAGTGTAGCC | |
| 788.2 with hBG | ATGCTCTAGGAAGATCAATTCAATTCACGCGTCCACGTTCTGCTTCACTCTCCCC | |
| exon/intron/exon | ATCTCCCCCCCCTCCCCACCCCCAATTTTGTATTTATTTATTTTTTAATTATTTT | |
| (construct 2) | GTGCAGCGATGGGGGCGGGGGGGGGGGGCGCGCGCCAGGCGGGGCGGGGCGGGGC | |
| | GAGGGGCGGGGCGGGGCGAGGCGGAGAGGTGCGGCGGCAGCCAATCAGAGCGGCG | |
| | CGCTCCGAAAGTTTCCTTTTATGGCGAGGCGGCGGCGGCGGCGGCCCTATAAAAA | |
| | GCGAAGCGCGCGGCGGGAAGCTTCGTTTAGTGAACCGTCAGATCGCCTGGAGACG | |
| | CCATCCACGCTGTTTTGACCTCCATAGAAGACACCGGGACCGATCCAGCCTCCGC | |
| | GGATTCGAATCCCGGCCGGGAACGGTGCATTGGAACGCGGATTCCCCGTGCCAAG | |
| | AGTGACGTAAGTACCGCCTATAGAGTCTATAGGCCCACAAAAAATGCTTTCTTCT | |
| | TTTAATATACTTTTTTGTTTATCTTATTTCTAATACTTTCCCTAATCTCTTTCTT | |
| | TCAGGGCAATAATGATACAATGTATCATGCCTCTTTGCACCATTCTAAAGAATAA | |
| | CAGTGATAATTTCTGGGTTAAGGCAATAGCAATATTTCTGCATATAAATATTTCT | |
| | GCATATAAATTGTAACTGATGTAAGAGGTTTCATATTGCTAATAGCAGCTACAAT | |
| | CCAGCTACCATTCTGCTTTTATTTTATGGTTGGGATAAGGCTGGATTATTCTGAG | |
| | TCCAAGCTAGGCCCTTTTGCTAATCATGTTCATACCTCTTATCTTCCTCCCACAG | |
| | CTCCTGGGCAACGTGCTGGTCTGTGTGCTGGCCCATCACTTTGGCAAAGAATTGG | |
| | GATTCGAACCGGTACCGAGCTCTCCCGCAGAACACCATGCGCTCCACGGAAGCAG | |
| | GTCCTCACTTTAATGCTGTGGCCACTGAGAAGTATTAAAGTGAGGACCTGCTTCT | |
| | GAGGAGCGCCTTGACAGCAGCCATGGGAGGGCCTCGAGGGGTGGCATCCCTGTGA | |
| | CCCCTCCCCAGTGCCTCTCCTGGCCCTGGAAGTTGCCACTCCAGTGCCCACCAGC | |
| | CTTGTCCTAATAAAATTAAGTTGCATCATTTTGTCTGACTAGGTGTCCTTCTATA | |
| | ATATTATGGGGTGGAGGGGGGTGGTATGGAGCAAGGGGCAAGTTGGGAAGACAAC | |
| | CTGTAGGGCCTGCGGGGTCTATTGGGAACCAAGCTGGAGTGCAGTGGCACAATCT | |
| | TGGCTCACTGCAATCTCCGCCTCCTGGGTTCAAGCGATTCTCCTGCCTCAGCCTC | |
| | CCGAGTTGTTGGGATTCCAGGCATGCATGACCAGGCTCAGCTAATTTTTGTTTTT | |
| | TTGGTAGAGACGGGGTTTCACCATATTGGCCAGGCTGGTCTCCAACTCCTAATCT | |
| | CAGGTGATCTACCCACCTTGGCCTCCCAAATTGCTGGGATTACAGGCGTGAACCA | |
| | CTGCTCCCTTCCCTGTCCTTATCGATGCCTAGGGTTAATCATTAACTACAAGGAA | |
| | CCCCTAGTGATGGAGTTGGCCACTCCCTCTCTGCGCGCTCGCTCGCTCACTGAGG | |
| | CCGGGCGACCAAAGGTCGCCCGACGCCCGGGCTTTGCCCGGGCGGCCTCAGTGAG | |
| | CGAGCGAGCGCGCAG | |
| ITR to ITR of | CTGCGCGCTCGCTCGCTCACTGAGGCCGCCCGGGCAAAGCCCGGGCGTCGGGCGA | 4030 |
| miniEF1aV2.mir104- | CCTTTGGTCGCCCGGCCTCAGTGAGCGAGCGAGCGCGCAGAGAGGGAGTGTAGCC | |
| 788.2 with hBG | ATGCTCTAGGAAGATCAATTCAATTCACGCGTCGTGAGGCTCCGGTGCCCGTCAG | |
| exon/intron/exon | TGGGCAGAGCGCACATCGCCCACAGTCCCCGAGAAGTTGGGGGGAGGGGTCGGCA | |
| (construct 3) | ATTGAACCGGTGCCTAGAGAAGGTGGCGCGGGGTAAACTGGGAAAGTGATGTCGT | |
| | GTACTGGCTCCGCCTTTTTCCCGAGGGTGGGGGAGAACCGTATATAAGTGCAGTA | |
| | GTCGCCGTGAACGTTCTTTTTCGCAACGGGTTTGCCGCCAGAACACAGAAGCTTC | |
| | GTTTAGTGAACCGTCAGATCGCCTGGAGACGCCATCCACGCTGTTTTGACCTCCA | |
| | TAGAAGACACCGGGACCGATCCAGCCTCCGCGGATTCGAATCCCGGCCGGGAACG | |
| | GTGCATTGGAACGCGGATTCCCCGTGCCAAGAGTGACGTAAGTACCGCCTATAGA | |
| | GTCTATAGGCCCACAAAAAATGCTTTCTTCTTTTAATATACTTTTTTGTTTATCT | |
| | TATTTCTAATACTTTCCCTAATCTCTTTCTTTCAGGGCAATAATGATACAATGTA | |
| | TCATGCCTCTTTGCACCATTCTAAAGAATAACAGTGATAATTTCTGGGTTAAGGC | |
| | AATAGCAATATTTCTGCATATAAATATTTCTGCATATAAATTGTAACTGATGTAA | |
| | GAGGTTTCATATTGCTAATAGCAGCTACAATCCAGCTACCATTCTGCTTTTATTT | |
| | TATGGTTGGGATAAGGCTGGATTATTCTGAGTCCAAGCTAGGCCCTTTTGCTAAT | |
| | CATGTTCATACCTCTTATCTTCCTCCCACAGCTCCTGGGCAACGTGCTGGTCTGT | |
| | GTGCTGGCCCATCACTTTGGCAAAGAATTGGGATTCGAACCGGTACCGAGCTCTC | |
| | CCGCAGAACACCATGCGCTCCACGGAAGCAGGTCCTCACTTTAATGCTGTGGCCA | |
| | CTGAGAAGTATTAAAGTGAGGACCTGCTTCTGAGGAGCGCCTTGACAGCAGCCAT | |
| | GGGAGGGCCTCGAGGGGTGGCATCCCTGTGACCCCTCCCCAGTGCCTCTCCTGGC | |
| | CCTGGAAGTTGCCACTCCAGTGCCCACCAGCCTTGTCCTAATAAAATTAAGTTGC | |
| | ATCATTTTGTCTGACTAGGTGTCCTTCTATAATATTATGGGGTGGAGGGGGGTGG | |
| | TATGGAGCAAGGGGCAAGTTGGGAAGACAACCTGTAGGGCCTGCGGGGTCTATTG | |
| | GGAACCAAGCTGGAGTGCAGTGGCACAATCTTGGCTCACTGCAATCTCCGCCTCC | |
| | TGGGTTCAAGCGATTCTCCTGCCTCAGCCTCCCGAGTTGTTGGGATTCCAGGCAT | |
| | GCATGACCAGGCTCAGCTAATTTTTGTTTTTTTGGTAGAGACGGGGTTTCACCAT | |
| | ATTGGCCAGGCTGGTCTCCAACTCCTAATCTCAGGTGATCTACCCACCTTGGCCT | |
| | CCCAAATTGCTGGGATTACAGGCGTGAACCACTGCTCCCTTCCCTGTCCTTATCG | |
| | ATGCCTAGGGTTAATCATTAACTACAAGGAACCCCTAGTGATGGAGTTGGCCACT | |
| | CCCTCTCTGCGCGCTCGCTCGCTCACTGAGGCCGGGCGACCAAAGGTCGCCCGAC | |
| | GCCCGGGCTTTGCCCGGGCGGCCTCAGTGAGCGAGCGAGCGCGCAG | |
| ITR to ITR of | CTGCGCGCTCGCTCGCTCACTGAGGCCGCCCGGGCAAAGCCCGGGCGTCGGGCGA | 4031 |
| PGK.mir104-788.2 | CCTTTGGTCGCCCGGCCTCAGTGAGCGAGCGAGCGCGCAGAGAGGGAGTGTAGCC | |
| with hBG | ATGCTCTAGGAAGATCAATTCAATTCACGCGTGGGTAGGGGAGGCGCTTTTCCCA | |
| exon/intron/exon | AGGCAGTCTGGAGCATGCGCTTTAGCAGCCCCGCTGGGCACTTGGCGCTACACAA | |
| (construct 4) | GTGGCCTCTGGCCTCGCACACATTCCACATCCACCGGTAGGCGCCAACCGGCTCC | |
| | GTTCTTTGGTGGCCCCTTCGCGCCACCTTCTACTCCTCCCCTAGTCAGGAAGTTC | |
| | CCCCCCGCCCCGCAGCTCGCGTCGTGCAGGACGTGACAAATGGAAGTAGCACGTC | |
| | TCACTAGTCTCGTGCAGATGGACAGCACCGCTGAGCAATGGAAGCGGGTAGGCCT | |
| | TTGGGGCAGCGGCCAATAGCAGCTTTGCTCCTTCGCTTTCTGGGCTCAGAGGCTG | |
| | GGAAGGGGTGGGTCCGGGGGCGGGCTCAGGGGCGGGCTCAGGGGCGGGGCGGGCG | |
| | CCCGAAGGTCCTCCGGAGGCCCGGCATTCTGCACGCTTCAAAAGCGCACGTCTGC | |
| | CGCGCTGTTCTCCTCTTCCTCATCTCCGGGCCTTTCGAAGCTTCGTTTAGTGAAC | |
| | CGTCAGATCGCCTGGAGACGCCATCCACGCTGTTTTGACCTCCATAGAAGACACC | |
| | GGGACCGATCCAGCCTCCGCGGATTCGAATCCCGGCCGGGAACGGTGCATTGGAA | |
| | CGCGGATTCCCCGTGCCAAGAGTGACGTAAGTACCGCCTATAGAGTCTATAGGCC | |
| | CACAAAAAATGCTTTCTTCTTTTAATATACTTTTTTGTTTATCTTATTTCTAATA | |
| | CTTTCCCTAATCTCTTTCTTTCAGGGCAATAATGATACAATGTATCATGCCTCTT | |
| | TGCACCATTCTAAAGAATAACAGTGATAATTTCTGGGTTAAGGCAATAGCAATAT | |
| | TTCTGCATATAAATATTTCTGCATATAAATTGTAACTGATGTAAGAGGTTTCATA | |
| | TTGCTAATAGCAGCTACAATCCAGCTACCATTCTGCTTTTATTTTATGGTTGGGA | |
| | TAAGGCTGGATTATTCTGAGTCCAAGCTAGGCCCTTTTGCTAATCATGTTCATAC | |
| | CTCTTATCTTCCTCCCACAGCTCCTGGGCAACGTGCTGGTCTGTGTGCTGGCCCA | |
| | TCACTTTGGCAAAGAATTGGGATTCGAACCGGTACCGAGCTCTCCCGCAGAACAC | |
| | CATGCGCTCCACGGAAGCAGGTCCTCACTTTAATGCTGTGGCCACTGAGAAGTAT | |
| | TAAAGTGAGGACCTGCTTCTGAGGAGCGCCTTGACAGCAGCCATGGGAGGGCCTC | |
| | GAGGGGTGGCATCCCTGTGACCCCTCCCCAGTGCCTCTCCTGGCCCTGGAAGTTG | |
| | CCACTCCAGTGCCCACCAGCCTTGTCCTAATAAAATTAAGTTGCATCATTTTGTC | |
| | TGACTAGGTGTCCTTCTATAATATTATGGGGTGGAGGGGGGTGGTATGGAGCAAG | |
| | GGGCAAGTTGGGAAGACAACCTGTAGGGCCTGCGGGGTCTATTGGGAACCAAGCT | |
| | GGAGTGCAGTGGCACAATCTTGGCTCACTGCAATCTCCGCCTCCTGGGTTCAAGC | |
| | GATTCTCCTGCCTCAGCCTCCCGAGTTGTTGGGATTCCAGGCATGCATGACCAGG | |
| | CTCAGCTAATTTTTGTTTTTTTGGTAGAGACGGGGTTTCACCATATTGGCCAGGC | |
| | TGGTCTCCAACTCCTAATCTCAGGTGATCTACCCACCTTGGCCTCCCAAATTGCT | |
| | GGGATTACAGGCGTGAACCACTGCTCCCTTCCCTGTCCTTATCGATGCCTAGGGT | |
| | TAATCATTAACTACAAGGAACCCCTAGTGATGGAGTTGGCCACTCCCTCTCTGCG | |
| | CGCTCGCTCGCTCACTGAGGCCGGGCGACCAAAGGTCGCCCGACGCCCGGGCTTT | |
| | GCCCGGGCGGCCTCAGTGAGCGAGCGAGCGCGCAG | |
| ITR to ITR of | CTGCGCGCTCGCTCGCTCACTGAGGCCGCCCGGGCAAAGCCCGGGCGTCGGGCGA | 4032 |
| MP84.mir104- | CCTTTGGTCGCCCGGCCTCAGTGAGCGAGCGAGCGCGCAGAGAGGGAGTGTAGCC | |
| 788.2 with hBG | ATGCTCTAGGAAGATCAATTCAATTCACGCGTCCCTAATGGGCCAGGCGGCAGGG | |
| exon/intron/exon | GTTGAGAGGTAGGGGAGATGGGCTCTGAGACTATAAAGCCAGCGGGGGCCCAGCA | |
| (construct 5) | GCCCTCAAGCTTCGTTTAGTGAACCGTCAGATCGCCTGGAGACGCCATCCACGCT | |
| | GTTTTGACCTCCATAGAAGACACCGGGACCGATCCAGCCTCCGCGGATTCGAATC | |
| | CCGGCCGGGAACGGTGCATTGGAACGCGGATTCCCCGTGCCAAGAGTGACGTAAG | |
| | TACCGCCTATAGAGTCTATAGGCCCACAAAAAATGCTTTCTTCTTTTAATATACT | |
| | TTTTTGTTTATCTTATTTCTAATACTTTCCCTAATCTCTTTCTTTCAGGGCAATA | |
| | ATGATACAATGTATCATGCCTCTTTGCACCATTCTAAAGAATAACAGTGATAATT | |
| | TCTGGGTTAAGGCAATAGCAATATTTCTGCATATAAATATTTCTGCATATAAATT | |
| | GTAACTGATGTAAGAGGTTTCATATTGCTAATAGCAGCTACAATCCAGCTACCAT | |
| | TCTGCTTTTATTTTATGGTTGGGATAAGGCTGGATTATTCTGAGTCCAAGCTAGG | |
| | CCCTTTTGCTAATCATGTTCATACCTCTTATCTTCCTCCCACAGCTCCTGGGCAA | |
| | CGTGCTGGTCTGTGTGCTGGCCCATCACTTTGGCAAAGAATTGGGATTCGAACCG | |
| | GTACCGAGCTCTCCCGCAGAACACCATGCGCTCCACGGAAGCAGGTCCTCACTTT | |
| | AATGCTGTGGCCACTGAGAAGTATTAAAGTGAGGACCTGCTTCTGAGGAGCGCCT | |
| | TGACAGCAGCCATGGGAGGGCCTCGAGGGGTGGCATCCCTGTGACCCCTCCCCAG | |
| | TGCCTCTCCTGGCCCTGGAAGTTGCCACTCCAGTGCCCACCAGCCTTGTCCTAAT | |
| | AAAATTAAGTTGCATCATTTTGTCTGACTAGGTGTCCTTCTATAATATTATGGGG | |
| | TGGAGGGGGGTGGTATGGAGCAAGGGGCAAGTTGGGAAGACAACCTGTAGGGCCT | |
| | GCGGGGTCTATTGGGAACCAAGCTGGAGTGCAGTGGCACAATCTTGGCTCACTGC | |
| | AATCTCCGCCTCCTGGGTTCAAGCGATTCTCCTGCCTCAGCCTCCCGAGTTGTTG | |
| | GGATTCCAGGCATGCATGACCAGGCTCAGCTAATTTTTGTTTTTTTGGTAGAGAC | |
| | GGGGTTTCACCATATTGGCCAGGCTGGTCTCCAACTCCTAATCTCAGGTGATCTA | |
| | CCCACCTTGGCCTCCCAAATTGCTGGGATTACAGGCGTGAACCACTGCTCCCTTC | |
| | CCTGTCCTTATCGATGCCTAGGGTTAATCATTAACTACAAGGAACCCCTAGTGAT | |
| | GGAGTTGGCCACTCCCTCTCTGCGCGCTCGCTCGCTCACTGAGGCCGGGCGACCA | |
| | AAGGTCGCCCGACGCCCGGGCTTTGCCCGGGCGGCCTCAGTGAGCGAGCGAGCGC | |
| | GCAG | |
| ITR to ITR of | CTGCGCGCTCGCTCGCTCACTGAGGCCGCCCGGGCAAAGCCCGGGCGTCGGGCGA | 4033 |
| hSYN(long).mir104- | CCTTTGGTCGCCCGGCCTCAGTGAGCGAGCGAGCGCGCAGAGAGGGAGTGTAGCC | |
| 788.2 with hBG | ATGCTCTAGGAAGATCAATTCAATTCACGCGTCGCGTGGGGTTATTTCTCTACTT | |
| exon/intron/exon | TCGTGTCTCTGAGTGTGCTTCCAGTGCCCCCCTCCCCCCAAAAAATGCCTTCTGA | |
| (construct 6) | GTTGAATATCAACACTACAAACCTAGTATCTGCAGAGGGCCCTGCGTATGAGTGC | |
| | AAGTGGGTTTTAGGACCAGGATGAGGCGGGGTGGGGGTGCCTACCTGACGACCGA | |
| | CCCCGACCCACTGGACAAGCACCCAACCCCCATTCCCCAAATTGCGCATCCCCTA | |
| | TCAGAGAGGGGGAGGGGAAACAGGATGCGGCGAGGCGCGTGCGCACTGCCAGCTT | |
| | CAGCACCGCGGACAGTGCCTTCGCCCCCGCCTGGCGGCGCGCGCCACCGCCGCCT | |
| | CAGCACTGAAGGCGCGCTGACGTCACTCGCCGGTCCCCCGCAAACTCCCCTTCCC | |
| | GGCCACCTTGGTCGCGTCCGCGCCGCCGCCGGCCCAGCCGGACCGCACCACGCGA | |
| | GGCGCGAGATAGGGGGGCACGGGCGCGACCATCTGCGCTGCGGCGCCGGCGACTC | |
| | AGCGCTGCCTCAGTCTGCGGTGGGCAGCGGAGGAGTCGTGTCGTGCCTGAGAGCG | |
| | CAGCTGTGCTCCTGGGCACCGCGCAGTCCGCCCCCGCGGCTCCTGGCCAGACCAC | |
| | CCCTAGGACCCCCTGCCCCAAGTCGCAGCCAAGCTTCGTTTAGTGAACCGTCAGA | |
| | TCGCCTGGAGACGCCATCCACGCTGTTTTGACCTCCATAGAAGACACCGGGACCG | |
| | ATCCAGCCTCCGCGGATTCGAATCCCGGCCGGGAACGGTGCATTGGAACGCGGAT | |
| | TCCCCGTGCCAAGAGTGACGTAAGTACCGCCTATAGAGTCTATAGGCCCACAAAA | |
| | AATGCTTTCTTCTTTTAATATACTTTTTTGTTTATCTTATTTCTAATACTTTCCC | |
| | TAATCTCTTTCTTTCAGGGCAATAATGATACAATGTATCATGCCTCTTTGCACCA | |
| | TTCTAAAGAATAACAGTGATAATTTCTGGGTTAAGGCAATAGCAATATTTCTGCA | |
| | TATAAATATTTCTGCATATAAATTGTAACTGATGTAAGAGGTTTCATATTGCTAA | |
| | TAGCAGCTACAATCCAGCTACCATTCTGCTTTTATTTTATGGTTGGGATAAGGCT | |
| | GGATTATTCTGAGTCCAAGCTAGGCCCTTTTGCTAATCATGTTCATACCTCTTAT | |
| | CTTCCTCCCACAGCTCCTGGGCAACGTGCTGGTCTGTGTGCTGGCCCATCACTTT | |
| | GGCAAAGAATTGGGATTCGAACCGGTACCGAGCTCTCCCGCAGAACACCATGCGC | |
| | TCCACGGAAGCAGGTCCTCACTTTAATGCTGTGGCCACTGAGAAGTATTAAAGTG | |
| | AGGACCTGCTTCTGAGGAGCGCCTTGACAGCAGCCATGGGAGGGCCTCGAGGGGT | |
| | GGCATCCCTGTGACCCCTCCCCAGTGCCTCTCCTGGCCCTGGAAGTTGCCACTCC | |
| | AGTGCCCACCAGCCTTGTCCTAATAAAATTAAGTTGCATCATTTTGTCTGACTAG | |
| | GTGTCCTTCTATAATATTATGGGGTGGAGGGGGGTGGTATGGAGCAAGGGGCAAG | |
| | TTGGGAAGACAACCTGTAGGGCCTGCGGGGTCTATTGGGAACCAAGCTGGAGTGC | |
| | AGTGGCACAATCTTGGCTCACTGCAATCTCCGCCTCCTGGGTTCAAGCGATTCTC | |
| | CTGCCTCAGCCTCCCGAGTTGTTGGGATTCCAGGCATGCATGACCAGGCTCAGCT | |
| | AATTTTTGTTTTTTTGGTAGAGACGGGGTTTCACCATATTGGCCAGGCTGGTCTC | |
| | CAACTCCTAATCTCAGGTGATCTACCCACCTTGGCCTCCCAAATTGCTGGGATTA | |
| | CAGGCGTGAACCACTGCTCCCTTCCCTGTCCTTATCGATGCCTAGGGTTAATCAT | |
| | TAACTACAAGGAACCCCTAGTGATGGAGTTGGCCACTCCCTCTCTGCGCGCTCGC | |
| | TCGCTCACTGAGGCCGGGCGACCAAAGGTCGCCCGACGCCCGGGCTTTGCCCGGG | |
| | CGGCCTCAGTGAGCGAGCGAGCGCGCAG | |
| ITR to ITR of | CTGCGCGCTCGCTCGCTCACTGAGGCCGCCCGGGCAAAGCCCGGGCGTCGGGCGA | 4034 |
| hSYN(short).mir104- | CCTTTGGTCGCCCGGCCTCAGTGAGCGAGCGAGCGCGCAGAGAGGGAGTGTAGCC | |
| 788.2 with hBG | ATGCTCTAGGAAGATCAATTCAATTCACGCGTTAGTATCTGCAGAGGGCCCTGCG | |
| exon/intron/exon | TATGAGTGCAAGTGGGTTTTAGGACCAGGATGAGGCGGGGTGGGGGTGCCTACCT | |
| (construct 7) | GACGACCGACCCCGACCCACTGGACAAGCACCCAACCCCCATTCCCCAAATTGCG | |
| | CATCCCCTATCAGAGAGGGGGAGGGGAAACAGGATGCGGCGAGGCGCGTGCGCAC | |
| | TGCCAGCTTCAGCACCGCGGACAGTGCCTTCGCCCCCGCCTGGCGGCGCGCGCCA | |
| | CCGCCGCCTCAGCACTGAAGGCGCGCTGACGTCACTCGCCGGTCCCCCGCAAACT | |
| | CCCCTTCCCGGCCACCTTGGTCGCGTCCGCGCCGCCGCCGGCCCAGCCGGACCGC | |
| | ACCACGCGAGGCGCGAGATAGGGGGGCACGGGCGCGACCATCTGCGCTGCGGCGC | |
| | CGGCGACTCAGCGCTGCCTCAGTCTGCGGTGGGCAGCGGAGGAGTCGTGTCGTGC | |
| | CTGAGAGCGCAGCTGTGCTCCTGGGCACCGCGCAGTCCGCCCCCGCGGCTCCTGG | |
| | CCAGACCACCCCTAGGACCCCCTGCCCCAAGTCGCAGCCAAGCTTCGTTTAGTGA | |
| | ACCGTCAGATCGCCTGGAGACGCCATCCACGCTGTTTTGACCTCCATAGAAGACA | |
| | CCGGGACCGATCCAGCCTCCGCGGATTCGAATCCCGGCCGGGAACGGTGCATTGG | |
| | AACGCGGATTCCCCGTGCCAAGAGTGACGTAAGTACCGCCTATAGAGTCTATAGG | |
| | CCCACAAAAAATGCTTTCTTCTTTTAATATACTTTTTTGTTTATCTTATTTCTAA | |
| | TACTTTCCCTAATCTCTTTCTTTCAGGGCAATAATGATACAATGTATCATGCCTC | |
| | TTTGCACCATTCTAAAGAATAACAGTGATAATTTCTGGGTTAAGGCAATAGCAAT | |
| | ATTTCTGCATATAAATATTTCTGCATATAAATTGTAACTGATGTAAGAGGTTTCA | |
| | TATTGCTAATAGCAGCTACAATCCAGCTACCATTCTGCTTTTATTTTATGGTTGG | |
| | GATAAGGCTGGATTATTCTGAGTCCAAGCTAGGCCCTTTTGCTAATCATGTTCAT | |
| | ACCTCTTATCTTCCTCCCACAGCTCCTGGGCAACGTGCTGGTCTGTGTGCTGGCC | |
| | CATCACTTTGGCAAAGAATTGGGATTCGAACCGGTACCGAGCTCTCCCGCAGAAC | |
| | ACCATGCGCTCCACGGAAGCAGGTCCTCACTTTAATGCTGTGGCCACTGAGAAGT | |
| | ATTAAAGTGAGGACCTGCTTCTGAGGAGCGCCTTGACAGCAGCCATGGGAGGGCC | |
| | TCGAGGGGTGGCATCCCTGTGACCCCTCCCCAGTGCCTCTCCTGGCCCTGGAAGT | |
| | TGCCACTCCAGTGCCCACCAGCCTTGTCCTAATAAAATTAAGTTGCATCATTTTG | |
| | TCTGACTAGGTGTCCTTCTATAATATTATGGGGTGGAGGGGGGTGGTATGGAGCA | |
| | AGGGGCAAGTTGGGAAGACAACCTGTAGGGCCTGCGGGGTCTATTGGGAACCAAG | |
| | CTGGAGTGCAGTGGCACAATCTTGGCTCACTGCAATCTCCGCCTCCTGGGTTCAA | |
| | GCGATTCTCCTGCCTCAGCCTCCCGAGTTGTTGGGATTCCAGGCATGCATGACCA | |
| | GGCTCAGCTAATTTTTGTTTTTTTGGTAGAGACGGGGTTTCACCATATTGGCCAG | |
| | GCTGGTCTCCAACTCCTAATCTCAGGTGATCTACCCACCTTGGCCTCCCAAATTG | |
| | CTGGGATTACAGGCGTGAACCACTGCTCCCTTCCCTGTCCTTATCGATGCCTAGG | |
| | GTTAATCATTAACTACAAGGAACCCCTAGTGATGGAGTTGGCCACTCCCTCTCTG | |
| | CGCGCTCGCTCGCTCACTGAGGCCGGGCGACCAAAGGTCGCCCGACGCCCGGGCT | |
| | TTGCCCGGGCGGCCTCAGTGAGCGAGCGAGCGCGCAG | |
| ITR to ITR of | CTGCGCGCTCGCTCGCTCACTGAGGCCGCCCGGGCAAAGCCCGGGCGTCGGGCGA | 4035 |
| CBA.mir104-788.2 | CCTTTGGTCGCCCGGCCTCAGTGAGCGAGCGAGCGCGCAGAGAGGGAGTGTAGCC | |
| with CpG deleted | ATGCTCTAGGAAGATCAATTCAATTCACGCGTCGACATTGATTATTGACTAGTTA | |
| hBG | TTAATAGTAATCAATTACGGGGTCATTAGTTCATAGCCCATATATGGAGTTCCGC | |
| exon/intron/exon | GTTACATAACTTACGGTAAATGGCCCGCCTGGCTGACCGCCCAACGACCCCCGCC | |
| (construct 8) | CATTGACGTCAATAATGACGTATGTTCCCATAGTAACGCCAATAGGGACTTTCCA | |
| | TTGACGTCAATGGGTGGAGTATTTACGGTAAACTGCCCACTTGGCAGTACATCAA | |
| | GTGTATCATATGCCAAGTACGCCCCCTATTGACGTCAATGACGGTAAATGGCCCG | |
| | CCTGGCATTATGCCCAGTACATGACCTTATGGGACTTTCCTACTTGGCAGTACAT | |
| | CTACGTATTAGTCATCGCTATTACCATGTCGAGGCCACGTTCTGCTTCACTCTCC | |
| | CCATCTCCCCCCCCTCCCCACCCCCAATTTTGTATTTATTTATTTTTTAATTATT | |
| | TTGTGCAGCGATGGGGGCGGGGGGGGGGGGCGCGCGCCAGGCGGGGCGGGGCGGG | |
| | GCGAGGGGCGGGGCGGGGCGAGGCGGAGAGGTGCGGCGGCAGCCAATCAGAGCGG | |
| | CGCGCTCCGAAAGTTTCCTTTTATGGCGAGGCGGCGGCGGCGGCGGCCCTATAAA | |
| | AAGCGAAGCGCGCGGCGGGCGGGAGCAAGCTTCGTTTAGTGAACCGTCAGATCAC | |
| | CTGGAGACACCATCCACACTGTTTTGACCTCCATAGAAGACACCAGGACCAATCC | |
| | AGCCTCCACAGATTCCAATCCCAGCCAGGAACAGTGCATTGGAACACAGATTCCC | |
| | CTTGCCAAGAGTGACGTAAGTACCGCCTATAGAGTCTATAGGCCCACAAAAAATG | |
| | CTTTCTTCTTTTAATATACTTTTTTGTTTATCTTATTTCTAATACTTTCCCTAAT | |
| | CTCTTTCTTTCAGGGCAATAATGATACAATGTATCATGCCTCTTTGCACCATTCT | |
| | AAAGAATAACAGTGATAATTTCTGGGTTAAGGCAATAGCAATATTTCTGCATATA | |
| | AATATTTCTGCATATAAATTGTAACTGATGTAAGAGGTTTCATATTGCTAATAGC | |
| | AGCTACAATCCAGCTACCATTCTGCTTTTATTTTATGGTTGGGATAAGGCTGGAT | |
| | TATTCTGAGTCCAAGCTAGGCCCTTTTGCTAATCATGTTCATACCTCTTATCTTC | |
| | CTCCCACAGCTCCTGGGCAACCTGCTGGTCTGTGTGCTGGCCCATCACTTTGGCA | |
| | AAGAATTGGGATTCGAACCGGTACCGAGCTCTCCCGCAGAACACCATGCGCTCCA | |
| | CGGAAGCAGGTCCTCACTTTAATGCTGTGGCCACTGAGAAGTATTAAAGTGAGGA | |
| | CCTGCTTCTGAGGAGCGCCTTGACAGCAGCCATGGGAGGGCCTCGAGGGGTGGCA | |
| | TCCCTGTGACCCCTCCCCAGTGCCTCTCCTGGCCCTGGAAGTTGCCACTCCAGTG | |
| | CCCACCAGCCTTGTCCTAATAAAATTAAGTTGCATCATTTTGTCTGACTAGGTGT | |
| | CCTTCTATAATATTATGGGGTGGAGGGGGGTGGTATGGAGCAAGGGGCAAGTTGG | |
| | GAAGACAACCTGTAGGGCCTGCGGGGTCTATTGGGAACCAAGCTGGAGTGCAGTG | |
| | GCACAATCTTGGCTCACTGCAATCTCCGCCTCCTGGGTTCAAGCGATTCTCCTGC | |
| | CTCAGCCTCCCGAGTTGTTGGGATTCCAGGCATGCATGACCAGGCTCAGCTAATT | |
| | TTTGTTTTTTTGGTAGAGACGGGGTTTCACCATATTGGCCAGGCTGGTCTCCAAC | |
| | TCCTAATCTCAGGTGATCTACCCACCTTGGCCTCCCAAATTGCTGGGATTACAGG | |
| | CGTGAACCACTGCTCCCTTCCCTGTCCTTATCGATGCCTAGGGTTAATCATTAAC | |
| | TACAAGGAACCCCTAGTGATGGAGTTGGCCACTCCCTCTCTGCGCGCTCGCTCGC | |
| | TCACTGAGGCCGGGCGACCAAAGGTCGCCCGACGCCCGGGCTTTGCCCGGGCGGC | |
| | CTCAGTGAGCGAGCGAGCGCGCAG | |
| ITR to ITR of | CTGCGCGCTCGCTCGCTCACTGAGGCCGCCCGGGCAAAGCCCGGGCGTCGGGCGA | 4036 |
| miniCBA.mir104- | CCTTTGGTCGCCCGGCCTCAGTGAGCGAGCGAGCGCGCAGAGAGGGAGTGTAGCC | |
| 788.2 with CpG | ATGCTCTAGGAAGATCAATTCAATTCACGCGTCCACGTTCTGCTTCACTCTCCCC | |
| deleted hBG | ATCTCCCCCCCCTCCCCACCCCCAATTTTGTATTTATTTATTTTTTAATTATTTT | |
| exon/intron/exon | GTGCAGCGATGGGGGCGGGGGGGGGGGGCGCGCGCCAGGCGGGGCGGGGCGGGGC | |
| (construct 9) | GAGGGGCGGGGCGGGGCGAGGCGGAGAGGTGCGGCGGCAGCCAATCAGAGCGGCG | |
| | CGCTCCGAAAGTTTCCTTTTATGGCGAGGCGGCGGCGGCGGCGGCCCTATAAAAA | |
| | GCGAAGCGCGCGGCGGGAAGCTTCGTTTAGTGAACCGTCAGATCACCTGGAGACA | |
| | CCATCCACACTGTTTTGACCTCCATAGAAGACACCAGGACCAATCCAGCCTCCAC | |
| | AGATTCCAATCCCAGCCAGGAACAGTGCATTGGAACACAGATTCCCCTTGCCAAG | |
| | AGTGACGTAAGTACCGCCTATAGAGTCTATAGGCCCACAAAAAATGCTTTCTTCT | |
| | TTTAATATACTTTTTTGTTTATCTTATTTCTAATACTTTCCCTAATCTCTTTCTT | |
| | TCAGGGCAATAATGATACAATGTATCATGCCTCTTTGCACCATTCTAAAGAATAA | |
| | CAGTGATAATTTCTGGGTTAAGGCAATAGCAATATTTCTGCATATAAATATTTCT | |
| | GCATATAAATTGTAACTGATGTAAGAGGTTTCATATTGCTAATAGCAGCTACAAT | |
| | CCAGCTACCATTCTGCTTTTATTTTATGGTTGGGATAAGGCTGGATTATTCTGAG | |
| | TCCAAGCTAGGCCCTTTTGCTAATCATGTTCATACCTCTTATCTTCCTCCCACAG | |
| | CTCCTGGGCAACCTGCTGGTCTGTGTGCTGGCCCATCACTTTGGCAAAGAATTGG | |
| | GATTCGAACCGGTACCGAGCTCTCCCGCAGAACACCATGCGCTCCACGGAAGCAG | |
| | GTCCTCACTTTAATGCTGTGGCCACTGAGAAGTATTAAAGTGAGGACCTGCTTCT | |
| | GAGGAGCGCCTTGACAGCAGCCATGGGAGGGCCTCGAGGGGTGGCATCCCTGTGA | |
| | CCCCTCCCCAGTGCCTCTCCTGGCCCTGGAAGTTGCCACTCCAGTGCCCACCAGC | |
| | CTTGTCCTAATAAAATTAAGTTGCATCATTTTGTCTGACTAGGTGTCCTTCTATA | |
| | ATATTATGGGGTGGAGGGGGGTGGTATGGAGCAAGGGGCAAGTTGGGAAGACAAC | |
| | CTGTAGGGCCTGCGGGGTCTATTGGGAACCAAGCTGGAGTGCAGTGGCACAATCT | |
| | TGGCTCACTGCAATCTCCGCCTCCTGGGTTCAAGCGATTCTCCTGCCTCAGCCTC | |
| | CCGAGTTGTTGGGATTCCAGGCATGCATGACCAGGCTCAGCTAATTTTTGTTTTT | |
| | TTGGTAGAGACGGGGTTTCACCATATTGGCCAGGCTGGTCTCCAACTCCTAATCT | |
| | CAGGTGATCTACCCACCTTGGCCTCCCAAATTGCTGGGATTACAGGCGTGAACCA | |
| | CTGCTCCCTTCCCTGTCCTTATCGATGCCTAGGGTTAATCATTAACTACAAGGAA | |
| | CCCCTAGTGATGGAGTTGGCCACTCCCTCTCTGCGCGCTCGCTCGCTCACTGAGG | |
| | CCGGGCGACCAAAGGTCGCCCGACGCCCGGGCTTTGCCCGGGCGGCCTCAGTGAG | |
| | CGAGCGAGCGCGCAG | |
| ITR to ITR of | CTGCGCGCTCGCTCGCTCACTGAGGCCGCCCGGGCAAAGCCCGGGCGTCGGGCGA | 4037 |
| miniEF1aV2.mir104- | CCTTTGGTCGCCCGGCCTCAGTGAGCGAGCGAGCGCGCAGAGAGGGAGTGTAGCC | |
| 788.2 with CpG | ATGCTCTAGGAAGATCAATTCAATTCACGCGTCGTGAGGCTCCGGTGCCCGTCAG | |
| deleted hBG | TGGGCAGAGCGCACATCGCCCACAGTCCCCGAGAAGTTGGGGGGAGGGGTCGGCA | |
| exon/intron/exon | ATTGAACCGGTGCCTAGAGAAGGTGGCGCGGGGTAAACTGGGAAAGTGATGTCGT | |
| (construct 10) | GTACTGGCTCCGCCTTTTTCCCGAGGGTGGGGGAGAACCGTATATAAGTGCAGTA | |
| | GTCGCCGTGAACGTTCTTTTTCGCAACGGGTTTGCCGCCAGAACACAGAAGCTTC | |
| | GTTTAGTGAACCGTCAGATCACCTGGAGACACCATCCACACTGTTTTGACCTCCA | |
| | TAGAAGACACCAGGACCAATCCAGCCTCCACAGATTCCAATCCCAGCCAGGAACA | |
| | GTGCATTGGAACACAGATTCCCCTTGCCAAGAGTGACGTAAGTACCGCCTATAGA | |
| | GTCTATAGGCCCACAAAAAATGCTTTCTTCTTTTAATATACTTTTTTGTTTATCT | |
| | TATTTCTAATACTTTCCCTAATCTCTTTCTTTCAGGGCAATAATGATACAATGTA | |
| | TCATGCCTCTTTGCACCATTCTAAAGAATAACAGTGATAATTTCTGGGTTAAGGC | |
| | AATAGCAATATTTCTGCATATAAATATTTCTGCATATAAATTGTAACTGATGTAA | |
| | GAGGTTTCATATTGCTAATAGCAGCTACAATCCAGCTACCATTCTGCTTTTATTT | |
| | TATGGTTGGGATAAGGCTGGATTATTCTGAGTCCAAGCTAGGCCCTTTTGCTAAT | |
| | CATGTTCATACCTCTTATCTTCCTCCCACAGCTCCTGGGCAACCTGCTGGTCTGT | |
| | GTGCTGGCCCATCACTTTGGCAAAGAATTGGGATTCGAACCGGTACCGAGCTCTC | |
| | CCGCAGAACACCATGCGCTCCACGGAAGCAGGTCCTCACTTTAATGCTGTGGCCA | |
| | CTGAGAAGTATTAAAGTGAGGACCTGCTTCTGAGGAGCGCCTTGACAGCAGCCAT | |
| | GGGAGGGCCTCGAGGGGTGGCATCCCTGTGACCCCTCCCCAGTGCCTCTCCTGGC | |
| | CCTGGAAGTTGCCACTCCAGTGCCCACCAGCCTTGTCCTAATAAAATTAAGTTGC | |
| | ATCATTTTGTCTGACTAGGTGTCCTTCTATAATATTATGGGGTGGAGGGGGGTGG | |
| | TATGGAGCAAGGGGCAAGTTGGGAAGACAACCTGTAGGGCCTGCGGGGTCTATTG | |
| | GGAACCAAGCTGGAGTGCAGTGGCACAATCTTGGCTCACTGCAATCTCCGCCTCC | |
| | TGGGTTCAAGCGATTCTCCTGCCTCAGCCTCCCGAGTTGTTGGGATTCCAGGCAT | |
| | GCATGACCAGGCTCAGCTAATTTTTGTTTTTTTGGTAGAGACGGGGTTTCACCAT | |
| | ATTGGCCAGGCTGGTCTCCAACTCCTAATCTCAGGTGATCTACCCACCTTGGCCT | |
| | CCCAAATTGCTGGGATTACAGGCGTGAACCACTGCTCCCTTCCCTGTCCTTATCG | |
| | ATGCCTAGGGTTAATCATTAACTACAAGGAACCCCTAGTGATGGAGTTGGCCACT | |
| | CCCTCTCTGCGCGCTCGCTCGCTCACTGAGGCCGGGCGACCAAAGGTCGCCCGAC | |
| | GCCCGGGCTTTGCCCGGGCGGCCTCAGTGAGCGAGCGAGCGCGCAG | |
| ITR to ITR of | CTGCGCGCTCGCTCGCTCACTGAGGCCGCCCGGGCAAAGCCCGGGCGTCGGGCGA | 4038 |
| PGK.mir104-788.2 | CCTTTGGTCGCCCGGCCTCAGTGAGCGAGCGAGCGCGCAGAGAGGGAGTGTAGCC | |
| with CpG deleted | ATGCTCTAGGAAGATCAATTCAATTCACGCGTGGGTAGGGGAGGCGCTTTTCCCA | |
| hBG | AGGCAGTCTGGAGCATGCGCTTTAGCAGCCCCGCTGGGCACTTGGCGCTACACAA | |
| exon/intron/exon | GTGGCCTCTGGCCTCGCACACATTCCACATCCACCGGTAGGCGCCAACCGGCTCC | |
| (construct 11) | GTTCTTTGGTGGCCCCTTCGCGCCACCTTCTACTCCTCCCCTAGTCAGGAAGTTC | |
| | CCCCCCGCCCCGCAGCTCGCGTCGTGCAGGACGTGACAAATGGAAGTAGCACGTC | |
| | TCACTAGTCTCGTGCAGATGGACAGCACCGCTGAGCAATGGAAGCGGGTAGGCCT | |
| | TTGGGGCAGCGGCCAATAGCAGCTTTGCTCCTTCGCTTTCTGGGCTCAGAGGCTG | |
| | GGAAGGGGTGGGTCCGGGGGCGGGCTCAGGGGCGGGCTCAGGGGCGGGGCGGGCG | |
| | CCCGAAGGTCCTCCGGAGGCCCGGCATTCTGCACGCTTCAAAAGCGCACGTCTGC | |
| | CGCGCTGTTCTCCTCTTCCTCATCTCCGGGCCTTTCGAAGCTTCGTTTAGTGAAC | |
| | CGTCAGATCACCTGGAGACACCATCCACACTGTTTTGACCTCCATAGAAGACACC | |
| | AGGACCAATCCAGCCTCCACAGATTCCAATCCCAGCCAGGAACAGTGCATTGGAA | |
| | CACAGATTCCCCTTGCCAAGAGTGACGTAAGTACCGCCTATAGAGTCTATAGGCC | |
| | CACAAAAAATGCTTTCTTCTTTTAATATACTTTTTTGTTTATCTTATTTCTAATA | |
| | CTTTCCCTAATCTCTTTCTTTCAGGGCAATAATGATACAATGTATCATGCCTCTT | |
| | TGCACCATTCTAAAGAATAACAGTGATAATTTCTGGGTTAAGGCAATAGCAATAT | |
| | TTCTGCATATAAATATTTCTGCATATAAATTGTAACTGATGTAAGAGGTTTCATA | |
| | TTGCTAATAGCAGCTACAATCCAGCTACCATTCTGCTTTTATTTTATGGTTGGGA | |
| | TAAGGCTGGATTATTCTGAGTCCAAGCTAGGCCCTTTTGCTAATCATGTTCATAC | |
| | CTCTTATCTTCCTCCCACAGCTCCTGGGCAACCTGCTGGTCTGTGTGCTGGCCCA | |
| | TCACTTTGGCAAAGAATTGGGATTCGAACCGGTACCGAGCTCTCCCGCAGAACAC | |
| | CATGCGCTCCACGGAAGCAGGTCCTCACTTTAATGCTGTGGCCACTGAGAAGTAT | |
| | TAAAGTGAGGACCTGCTTCTGAGGAGCGCCTTGACAGCAGCCATGGGAGGGCCTC | |
| | GAGGGGTGGCATCCCTGTGACCCCTCCCCAGTGCCTCTCCTGGCCCTGGAAGTTG | |
| | CCACTCCAGTGCCCACCAGCCTTGTCCTAATAAAATTAAGTTGCATCATTTTGTC | |
| | TGACTAGGTGTCCTTCTATAATATTATGGGGTGGAGGGGGGTGGTATGGAGCAAG | |
| | GGGCAAGTTGGGAAGACAACCTGTAGGGCCTGCGGGGTCTATTGGGAACCAAGCT | |
| | GGAGTGCAGTGGCACAATCTTGGCTCACTGCAATCTCCGCCTCCTGGGTTCAAGC | |
| | GATTCTCCTGCCTCAGCCTCCCGAGTTGTTGGGATTCCAGGCATGCATGACCAGG | |
| | CTCAGCTAATTTTTGTTTTTTTGGTAGAGACGGGGTTTCACCATATTGGCCAGGC | |
| | TGGTCTCCAACTCCTAATCTCAGGTGATCTACCCACCTTGGCCTCCCAAATTGCT | |
| | GGGATTACAGGCGTGAACCACTGCTCCCTTCCCTGTCCTTATCGATGCCTAGGGT | |
| | TAATCATTAACTACAAGGAACCCCTAGTGATGGAGTTGGCCACTCCCTCTCTGCG | |
| | CGCTCGCTCGCTCACTGAGGCCGGGCGACCAAAGGTCGCCCGACGCCCGGGCTTT | |
| | GCCCGGGCGGCCTCAGTGAGCGAGCGAGCGCGCAG | |
| ITR to ITR of | CTGCGCGCTCGCTCGCTCACTGAGGCCGCCCGGGCAAAGCCCGGGCGTCGGGCGA | 4039 |
| MP84.mir104- | CCTTTGGTCGCCCGGCCTCAGTGAGCGAGCGAGCGCGCAGAGAGGGAGTGTAGCC | |
| 788.2 with CpG | ATGCTCTAGGAAGATCAATTCAATTCACGCGTCCCTAATGGGCCAGGCGGCAGGG | |
| deleted hBG | GTTGAGAGGTAGGGGAGATGGGCTCTGAGACTATAAAGCCAGCGGGGGCCCAGCA | |
| exon/intron/exon | GCCCTCAAGCTTCGTTTAGTGAACCGTCAGATCACCTGGAGACACCATCCACACT | |
| (construct 12) | GTTTTGACCTCCATAGAAGACACCAGGACCAATCCAGCCTCCACAGATTCCAATC | |
| | CCAGCCAGGAACAGTGCATTGGAACACAGATTCCCCTTGCCAAGAGTGACGTAAG | |
| | TACCGCCTATAGAGTCTATAGGCCCACAAAAAATGCTTTCTTCTTTTAATATACT | |
| | TTTTTGTTTATCTTATTTCTAATACTTTCCCTAATCTCTTTCTTTCAGGGCAATA | |
| | ATGATACAATGTATCATGCCTCTTTGCACCATTCTAAAGAATAACAGTGATAATT | |
| | TCTGGGTTAAGGCAATAGCAATATTTCTGCATATAAATATTTCTGCATATAAATT | |
| | GTAACTGATGTAAGAGGTTTCATATTGCTAATAGCAGCTACAATCCAGCTACCAT | |
| | TCTGCTTTTATTTTATGGTTGGGATAAGGCTGGATTATTCTGAGTCCAAGCTAGG | |
| | CCCTTTTGCTAATCATGTTCATACCTCTTATCTTCCTCCCACAGCTCCTGGGCAA | |
| | CCTGCTGGTCTGTGTGCTGGCCCATCACTTTGGCAAAGAATTGGGATTCGAACCG | |
| | GTACCGAGCTCTCCCGCAGAACACCATGCGCTCCACGGAAGCAGGTCCTCACTTT | |
| | AATGCTGTGGCCACTGAGAAGTATTAAAGTGAGGACCTGCTTCTGAGGAGCGCCT | |
| | TGACAGCAGCCATGGGAGGGCCTCGAGGGGTGGCATCCCTGTGACCCCTCCCCAG | |
| | TGCCTCTCCTGGCCCTGGAAGTTGCCACTCCAGTGCCCACCAGCCTTGTCCTAAT | |
| | AAAATTAAGTTGCATCATTTTGTCTGACTAGGTGTCCTTCTATAATATTATGGGG | |
| | TGGAGGGGGGTGGTATGGAGCAAGGGGCAAGTTGGGAAGACAACCTGTAGGGCCT | |
| | GCGGGGTCTATTGGGAACCAAGCTGGAGTGCAGTGGCACAATCTTGGCTCACTGC | |
| | AATCTCCGCCTCCTGGGTTCAAGCGATTCTCCTGCCTCAGCCTCCCGAGTTGTTG | |
| | GGATTCCAGGCATGCATGACCAGGCTCAGCTAATTTTTGTTTTTTTGGTAGAGAC | |
| | GGGGTTTCACCATATTGGCCAGGCTGGTCTCCAACTCCTAATCTCAGGTGATCTA | |
| | CCCACCTTGGCCTCCCAAATTGCTGGGATTACAGGCGTGAACCACTGCTCCCTTC | |
| | CCTGTCCTTATCGATGCCTAGGGTTAATCATTAACTACAAGGAACCCCTAGTGAT | |
| | GGAGTTGGCCACTCCCTCTCTGCGCGCTCGCTCGCTCACTGAGGCCGGGCGACCA | |
| | AAGGTCGCCCGACGCCCGGGCTTTGCCCGGGCGGCCTCAGTGAGCGAGCGAGCGC | |
| | GCAG | |
| ITR to ITR of | CTGCGCGCTCGCTCGCTCACTGAGGCCGCCCGGGCAAAGCCCGGGCGTCGGGCGA | 4040 |
| hSYN(long).mir104- | CCTTTGGTCGCCCGGCCTCAGTGAGCGAGCGAGCGCGCAGAGAGGGAGTGTAGCC | |
| 788.2 with CpG | ATGCTCTAGGAAGATCAATTCAATTCACGCGTCGCGTGGGGTTATTTCTCTACTT | |
| deleted hBG | TCGTGTCTCTGAGTGTGCTTCCAGTGCCCCCCTCCCCCCAAAAAATGCCTTCTGA | |
| exon/intron/exon | GTTGAATATCAACACTACAAACCTAGTATCTGCAGAGGGCCCTGCGTATGAGTGC | |
| (construct 13) | AAGTGGGTTTTAGGACCAGGATGAGGCGGGGTGGGGGTGCCTACCTGACGACCGA | |
| | CCCCGACCCACTGGACAAGCACCCAACCCCCATTCCCCAAATTGCGCATCCCCTA | |
| | TCAGAGAGGGGGAGGGGAAACAGGATGCGGCGAGGCGCGTGCGCACTGCCAGCTT | |
| | CAGCACCGCGGACAGTGCCTTCGCCCCCGCCTGGCGGCGCGCGCCACCGCCGCCT | |
| | CAGCACTGAAGGCGCGCTGACGTCACTCGCCGGTCCCCCGCAAACTCCCCTTCCC | |
| | GGCCACCTTGGTCGCGTCCGCGCCGCCGCCGGCCCAGCCGGACCGCACCACGCGA | |
| | GGCGCGAGATAGGGGGGCACGGGCGCGACCATCTGCGCTGCGGCGCCGGCGACTC | |
| | AGCGCTGCCTCAGTCTGCGGTGGGCAGCGGAGGAGTCGTGTCGTGCCTGAGAGCG | |
| | CAGCTGTGCTCCTGGGCACCGCGCAGTCCGCCCCCGCGGCTCCTGGCCAGACCAC | |
| | CCCTAGGACCCCCTGCCCCAAGTCGCAGCCAAGCTTCGTTTAGTGAACCGTCAGA | |
| | TCACCTGGAGACACCATCCACACTGTTTTGACCTCCATAGAAGACACCAGGACCA | |
| | ATCCAGCCTCCACAGATTCCAATCCCAGCCAGGAACAGTGCATTGGAACACAGAT | |
| | TCCCCTTGCCAAGAGTGACGTAAGTACCGCCTATAGAGTCTATAGGCCCACAAAA | |
| | AATGCTTTCTTCTTTTAATATACTTTTTTGTTTATCTTATTTCTAATACTTTCCC | |
| | TAATCTCTTTCTTTCAGGGCAATAATGATACAATGTATCATGCCTCTTTGCACCA | |
| | TTCTAAAGAATAACAGTGATAATTTCTGGGTTAAGGCAATAGCAATATTTCTGCA | |
| | TATAAATATTTCTGCATATAAATTGTAACTGATGTAAGAGGTTTCATATTGCTAA | |
| | TAGCAGCTACAATCCAGCTACCATTCTGCTTTTATTTTATGGTTGGGATAAGGCT | |
| | GGATTATTCTGAGTCCAAGCTAGGCCCTTTTGCTAATCATGTTCATACCTCTTAT | |
| | CTTCCTCCCACAGCTCCTGGGCAACCTGCTGGTCTGTGTGCTGGCCCATCACTTT | |
| | GGCAAAGAATTGGGATTCGAACCGGTACCGAGCTCTCCCGCAGAACACCATGCGC | |
| | TCCACGGAAGCAGGTCCTCACTTTAATGCTGTGGCCACTGAGAAGTATTAAAGTG | |
| | AGGACCTGCTTCTGAGGAGCGCCTTGACAGCAGCCATGGGAGGGCCTCGAGGGGT | |
| | GGCATCCCTGTGACCCCTCCCCAGTGCCTCTCCTGGCCCTGGAAGTTGCCACTCC | |
| | AGTGCCCACCAGCCTTGTCCTAATAAAATTAAGTTGCATCATTTTGTCTGACTAG | |
| | GTGTCCTTCTATAATATTATGGGGTGGAGGGGGGTGGTATGGAGCAAGGGGCAAG | |
| | TTGGGAAGACAACCTGTAGGGCCTGCGGGGTCTATTGGGAACCAAGCTGGAGTGC | |
| | AGTGGCACAATCTTGGCTCACTGCAATCTCCGCCTCCTGGGTTCAAGCGATTCTC | |
| | CTGCCTCAGCCTCCCGAGTTGTTGGGATTCCAGGCATGCATGACCAGGCTCAGCT | |
| | AATTTTTGTTTTTTTGGTAGAGACGGGGTTTCACCATATTGGCCAGGCTGGTCTC | |
| | CAACTCCTAATCTCAGGTGATCTACCCACCTTGGCCTCCCAAATTGCTGGGATTA | |
| | CAGGCGTGAACCACTGCTCCCTTCCCTGTCCTTATCGATGCCTAGGGTTAATCAT | |
| | TAACTACAAGGAACCCCTAGTGATGGAGTTGGCCACTCCCTCTCTGCGCGCTCGC | |
| | TCGCTCACTGAGGCCGGGCGACCAAAGGTCGCCCGACGCCCGGGCTTTGCCCGGG | |
| | CGGCCTCAGTGAGCGAGCGAGCGCGCAG | |
| ITR to ITR of | CTGCGCGCTCGCTCGCTCACTGAGGCCGCCCGGGCAAAGCCCGGGCGTCGGGCGA | 4041 |
| hSYN(short).mir104- | CCTTTGGTCGCCCGGCCTCAGTGAGCGAGCGAGCGCGCAGAGAGGGAGTGTAGCC | |
| 788.2 with CpG | ATGCTCTAGGAAGATCAATTCAATTCACGCGTTAGTATCTGCAGAGGGCCCTGCG | |
| deleted hBG | TATGAGTGCAAGTGGGTTTTAGGACCAGGATGAGGCGGGGTGGGGGTGCCTACCT | |
| exon/intron/exon | GACGACCGACCCCGACCCACTGGACAAGCACCCAACCCCCATTCCCCAAATTGCG | |
| (construct 14) | CATCCCCTATCAGAGAGGGGGAGGGGAAACAGGATGCGGCGAGGCGCGTGCGCAC | |
| | TGCCAGCTTCAGCACCGCGGACAGTGCCTTCGCCCCCGCCTGGCGGCGCGCGCCA | |
| | CCGCCGCCTCAGCACTGAAGGCGCGCTGACGTCACTCGCCGGTCCCCCGCAAACT | |
| | CCCCTTCCCGGCCACCTTGGTCGCGTCCGCGCCGCCGCCGGCCCAGCCGGACCGC | |
| | ACCACGCGAGGCGCGAGATAGGGGGGCACGGGCGCGACCATCTGCGCTGCGGCGC | |
| | CGGCGACTCAGCGCTGCCTCAGTCTGCGGTGGGCAGCGGAGGAGTCGTGTCGTGC | |
| | CTGAGAGCGCAGCTGTGCTCCTGGGCACCGCGCAGTCCGCCCCCGCGGCTCCTGG | |
| | CCAGACCACCCCTAGGACCCCCTGCCCCAAGTCGCAGCCAAGCTTCGTTTAGTGA | |
| | ACCGTCAGATCACCTGGAGACACCATCCACACTGTTTTGACCTCCATAGAAGACA | |
| | CCAGGACCAATCCAGCCTCCACAGATTCCAATCCCAGCCAGGAACAGTGCATTGG | |
| | AACACAGATTCCCCTTGCCAAGAGTGACGTAAGTACCGCCTATAGAGTCTATAGG | |
| | CCCACAAAAAATGCTTTCTTCTTTTAATATACTTTTTTGTTTATCTTATTTCTAA | |
| | TACTTTCCCTAATCTCTTTCTTTCAGGGCAATAATGATACAATGTATCATGCCTC | |
| | TTTGCACCATTCTAAAGAATAACAGTGATAATTTCTGGGTTAAGGCAATAGCAAT | |
| | ATTTCTGCATATAAATATTTCTGCATATAAATTGTAACTGATGTAAGAGGTTTCA | |
| | TATTGCTAATAGCAGCTACAATCCAGCTACCATTCTGCTTTTATTTTATGGTTGG | |
| | GATAAGGCTGGATTATTCTGAGTCCAAGCTAGGCCCTTTTGCTAATCATGTTCAT | |
| | ACCTCTTATCTTCCTCCCACAGCTCCTGGGCAACCTGCTGGTCTGTGTGCTGGCC | |
| | CATCACTTTGGCAAAGAATTGGGATTCGAACCGGTACCGAGCTCTCCCGCAGAAC | |
| | ACCATGCGCTCCACGGAAGCAGGTCCTCACTTTAATGCTGTGGCCACTGAGAAGT | |
| | ATTAAAGTGAGGACCTGCTTCTGAGGAGCGCCTTGACAGCAGCCATGGGAGGGCC | |
| | TCGAGGGGTGGCATCCCTGTGACCCCTCCCCAGTGCCTCTCCTGGCCCTGGAAGT | |
| | TGCCACTCCAGTGCCCACCAGCCTTGTCCTAATAAAATTAAGTTGCATCATTTTG | |
| | TCTGACTAGGTGTCCTTCTATAATATTATGGGGTGGAGGGGGGTGGTATGGAGCA | |
| | AGGGGCAAGTTGGGAAGACAACCTGTAGGGCCTGCGGGGTCTATTGGGAACCAAG | |
| | CTGGAGTGCAGTGGCACAATCTTGGCTCACTGCAATCTCCGCCTCCTGGGTTCAA | |
| | GCGATTCTCCTGCCTCAGCCTCCCGAGTTGTTGGGATTCCAGGCATGCATGACCA | |
| | GGCTCAGCTAATTTTTGTTTTTTTGGTAGAGACGGGGTTTCACCATATTGGCCAG | |
| | GCTGGTCTCCAACTCCTAATCTCAGGTGATCTACCCACCTTGGCCTCCCAAATTG | |
| | CTGGGATTACAGGCGTGAACCACTGCTCCCTTCCCTGTCCTTATCGATGCCTAGG | |
| | GTTAATCATTAACTACAAGGAACCCCTAGTGATGGAGTTGGCCACTCCCTCTCTG | |
| | CGCGCTCGCTCGCTCACTGAGGCCGGGCGACCAAAGGTCGCCCGACGCCCGGGCT | |
| | TTGCCCGGGCGGCCTCAGTGAGCGAGCGAGCGCGCAG | |

TABLE 18
Summary of components of the ITR to ITR sequences of AAV Particles in Table 17

| | | | hβG | | hGH poly | |
|---|---|---|---|---|---|---|
| Construct (from | | | Exon/Intron/ | Modulatory | A signal | |
| Table 17) | 5′ITR | Promoter | Exon | polynucleotide | sequence | 3′ITR |
| 1 | SEQ | SEQ ID: | SEQ ID NO: | SEQ ID NO: | SEQ ID | SEQ ID: |
| (SEQ ID NO: 4028) | ID: 126 | 4021 | 4042 | 2562 | NO: 4027 | 130 |
| 2 | SEQ | SEQ ID: | SEQ ID NO: | SEQ ID NO: | SEQ ID | SEQ ID: |
| (SEQ ID NO: 4029) | ID: 126 | 4022 | 4042 | 2562 | NO: 4027 | 130 |
| 3 | SEQ | SEQ ID: | SEQ ID NO: | SEQ ID NO: | SEQ ID | SEQ ID: |
| (SEQ ID NO: 4030) | ID: 126 | 4014 | 4042 | 2562 | NO: 4027 | 130 |
| 4 | SEQ | SEQ ID: | SEQ ID NO: | SEQ ID NO: | SEQ ID | SEQ ID: |
| (SEQ ID NO: 4031) | ID: 126 | 4023 | 4042 | 2562 | NO: 4027 | 130 |
| 5 | SEQ | SEQ ID: | SEQ ID NO: | SEQ ID NO: | SEQ ID | SEQ ID: |
| (SEQ ID NO: 4032) | ID: 126 | 4024 | 4042 | 2562 | NO: 4027 | 130 |
| 6 | SEQ | SEQ ID: | SEQ ID NO: | SEQ ID NO: | SEQ ID | SEQ ID: |
| (SEQ ID NO: 4033) | ID: 126 | 4025 | 4042 | 2562 | NO: 4027 | 130 |
| 7 | SEQ | SEQ ID: | SEQ ID NO: | SEQ ID NO: | SEQ ID | SEQ ID: |
| (SEQ ID NO: 4034) | ID: 126 | 4026 | 4042 | 2562 | NO: 4027 | 130 |
| 8 | SEQ | SEQ ID: | SEQ ID NO: | SEQ ID NO: | SEQ ID | SEQ ID: |
| (SEQ ID NO: 4035) | ID: 126 | 4021 | 4043 | 2562 | NO: 4027 | 130 |
| 9 | SEQ | SEQ ID: | SEQ ID NO: | SEQ ID NO: | SEQ ID | SEQ ID: |
| (SEQ ID NO: 4036) | ID: 126 | 4022 | 4043 | 2562 | NO: 4027 | 130 |
| 10 | SEQ | SEQ ID: | SEQ ID NO: | SEQ ID NO: | SEQ ID | SEQ ID: |
| (SEQ ID NO: 4037) | ID: 126 | 4014 | 4043 | 2562 | NO: 4027 | 130 |
| 11 | SEQ | SEQ ID: | SEQ ID NO: | SEQ ID NO: | SEQ ID | SEQ ID: |
| (SEQ ID NO: 4038) | ID: 126 | 4023 | 4043 | 2562 | NO: 4027 | 130 |
| 12 | SEQ | SEQ ID: | SEQ ID NO: | SEQ ID NO: | SEQ ID | SEQ ID: |
| (SEQ ID NO: 4039) | ID: 126 | 4024 | 4043 | 2562 | NO: 4027 | 130 |
| 13 | SEQ | SEQ ID: | SEQ ID NO: | SEQ ID NO: | SEQ ID | SEQ ID: |
| (SEQ ID NO: 4040) | ID: 126 | 4025 | 4043 | 2562 | NO: 4027 | 130 |
| 14 | SEQ | SEQ ID: | SEQ ID NO: | SEQ ID NO: | SEQ ID | SEQ ID: |
| (SEQ ID NO: 4041) | ID: 126 | 4026 | 4043 | 2562 | NO: 4027 | 130 |

In certain embodiments, the AAV particle comprises a viral genome which comprises a sequence which has a percent identity to SEQ ID NO: 109. The viral genome may have at least 70%, 75%, 80%, 85%, 90%, 95%, 99% or 100% identity to SEQ ID NO: 9. The viral genome may have 70-80%, 70-90%, 70-99%, 70-100%, 80-85%, 80-90%, 80-95%, 80-99%, 80-100%, 90-95%, 90-99%, or 90-100% identity to SEQ ID NO: 109. As a non-limiting example, the viral genome comprises a sequence which has at least 80% identity to SEQ ID NO: 109. As another non-limiting example, the viral genome comprises a sequence which has at least 85% identity to SEQ ID NO: 109. As another non-limiting example, the viral genome comprises a sequence which has at least 90% identity to SEQ ID NO: 109. As another non-limiting example, the viral genome comprises a sequence which has at least 95% identity to SEQ ID NO: 109. As another non-limiting example, the viral genome comprises a sequence which has at least 99% identity to SEQ ID NO: 109. In some embodiments, the AAV particle comprises the viral genome sequence of SEQ ID NO: 109 differing by no more than 15, 14, 13, 12, 11, 10, 9, 8, 7, 6, 5, 4, 3, 2 or 1 nucleotides from SEQ ID NO: 109. These differing nucleotides may be contiguous, or alternatively, they may be non-contiguous throughout the sequence. In some embodiments, the differing nucleotides are located at the ends of the sequences.

In certain embodiments, the AAV particle comprises a viral genome which comprises a sequence which has a percent identity to SEQ ID NO: 125. The viral genome may have at least 70%, 75%, 80%, 85%, 90%, 95%, 99% or 100% identity to SEQ ID NO: 125. The viral genome may have 70-80%, 70-90%, 70-99%, 70-100%, 80-85%, 80-90%, 80-95%, 80-99%, 80-100%, 90-95%, 90-99%, or 90-100% identity to SEQ ID NO: 125. As a non-limiting example, the viral genome comprises a sequence which has at least 80% identity to SEQ ID NO: 125. As another non-limiting example, the viral genome comprises a sequence which has at least 85% identity to SEQ ID NO: 125. As another non-limiting example, the viral genome comprises a sequence which has at least 90% identity to SEQ ID NO: 125. As another non-limiting example, the viral genome comprises a sequence which has at least 95% identity to SEQ ID NO: 125. As another non-limiting example, the viral genome comprises a sequence which has at least 99% identity to SEQ ID NO: 125. In some embodiments, the AAV particle comprises the viral genome sequence of SEQ ID NO: 125 differing by no more than 15, 14, 13, 12, 11, 10, 9, 8, 7, 6, 5, 4, 3, 2 or 1 nucleotides from SEQ ID NO: 125. These differing nucleotides may be contiguous, or alternatively, they may be non-contiguous throughout the sequence. In some embodiments, the differing nucleotides are located at the ends of the sequences.

In certain embodiments, the AAV particle comprises a viral genome which comprises a nucleotide sequence which has a percent identity to any one of SEQ ID NOs: 4028-4041. The viral genome may have at least 70%, 75%, 80%, 85%, 90%, 95%, 99% or 100% identity to any one of SEQ ID NOs: 4028-4041. The viral genome may have 70-80%, 70-90%, 70-99%, 70-100%, 80-85%, 80-90%, 80-95%, 80-99%, 80-100%, 90-95%, 90-99%, or 90-100% identity to any one of SEQ ID NOs: 4028-4041. As a non-limiting example, the viral genome comprises a nucleotide sequence which has at least 80% identity to any one of SEQ ID NOs: 4028-4041. As another non-limiting example, the viral genome comprises a nucleotide sequence which has at least 85% identity to any one of SEQ ID NOs: 4028-4041. As another non-limiting example, the viral genome comprises a nucleotide sequence which has at least 90% identity to any one of SEQ ID NOs: 4028-4041. As another non-limiting example, the viral genome comprises a nucleotide sequence which has at least 95% identity to any one of SEQ ID NOs: 4028-4041. As another non-limiting example, the viral genome comprises a nucleotide sequence which has at least 99% identity to any one of SEQ ID NOs: 4028-4041. In some embodiments, the AAV particle comprises a viral genome sequence differing by no more than 15, 14, 13, 12, 11, 10, 9, 8, 7, 6, 5, 4, 3, 2 or 1 nucleotides relative to any one of SEQ ID NOs: 4028-4041. These differing nucleotides may be contiguous, or alternatively, they may be non-contiguous throughout the sequence. In some embodiments, the differing nucleotides are located at the ends of the sequences.

In some embodiments, the modulatory polynucleotide sequence of any one of SEQ ID NOs: 4028-4041 is located within (e.g., inserted into) the intron, or replaces a part or all of the intron, of the hBG exon-intron-exon cassette rather than being located 3′ relative to the hBG exon-intron-exon cassette. In some embodiments, one or more of the 5′ ITR, promoter, exon-intron-exon cassette, nucleotide sequence encoding the modulatory polynucleotide, polyA signal sequence, and 3′ITR of the ITR to ITR sequences of any one of SEQ ID NOs: 4028-4041 in Tables 17 and 18 can be replaced with a variant 5′ ITR, promoter, exon-intron-exon cassette, nucleotide sequence encoding the modulatory polynucleotide, polyA signal sequence, and/or 3′ITR sequence, e.g., one or more variant components comprising a nucleotide sequence having at least one, two, or three but no more than four modifications (e.g., substitutions, insertions, and/or deletions) relative to the nucleotide sequence of the non-modified version of the component or components, or a nucleotide sequence with at least 70% (e.g., 80, 85%, 90%, 95%, 96%, 97%, 98%, or 99%) sequence identity to the non-modified version of the component or components.

In some embodiments, the AAV particle comprises a viral genome which comprises, from 5′ to 3′: a 5′ ITR, a promoter (e.g., promoter of CBA, mini-CBA, EF-1, PGK, MP84, SYN, or a variant (e.g., truncated version) of any of the preceding promoters), an exon-intron-exon cassette (e.g., from hBG), a nucleotide sequence encoding a modulatory polynucleotide, wherein the modulatory polynucleotide comprises an siRNA encoding sequence which targets SOD1, a polyA signal sequence (e.g., an rBGpA or hGH poly A signal sequence), and 3′ITR.

In some embodiments, the AAV particle comprises a viral genome which comprises, from 5′ to 3′: a 5′ ITR, a promoter (e.g., promoter of CBA, mini-CBA, EF-1, PGK, MP84, SYN, or a variant (e.g., truncated version) of any of the preceding promoters), an exon-intron-exon cassette (e.g., from hBG), a nucleotide sequence encoding a modulatory polynucleotide comprising an siRNA encoding sequence which targets SOD1 (e.g., hSOD1), wherein the modulatory polynucleotide is inserted into or replaces a portion or all of the intron in the exon-intron-exon cassette, a polyA signal sequence (e.g., an rBGpA or hGH poly A signal sequence), and 3′ITR.

In some embodiments, AAV particle comprises a viral genome which comprises, from 5′ to 3′: a 5′ ITR sequence comprising, e.g., SEQ ID NO: 126, a promoter sequence selected from, e.g., SEQ ID NOs: 4000-4026, an exon-intron-exon cassette selected from e.g., SEQ ID NO: 4042 or 4043, a nucleotide sequence encoding a modulatory polynucleotide comprising an siRNA encoding sequence which targets SOD1 (e.g., hSOD1), a poly A signal sequence selected from, e.g., SEQ ID NO: 129 or 4027, and a 3′ ITR sequence comprising, e.g., SEQ ID NO: 130.

In some embodiments, AAV particle comprises a viral genome which comprises, from 5′ to 3′: a 5′ ITR sequence comprising, e.g., SEQ ID NO: 126, a promoter sequence selected from, e.g., SEQ ID NOs: 4000-4026, an exon-intron-exon cassette selected from e.g., SEQ ID NO: 4042 or 4043, a nucleotide sequence encoding a modulatory polynucleotide comprising an siRNA encoding sequence which targets SOD1 (e.g., hSOD1), wherein the modulatory polynucleotide is inserted into or replaces a portion or all of the intron in the exon-intron-exon cassette, a poly A signal sequence selected from, e.g., SEQ ID NO: 129 or 4027, and a 3′ ITR sequence comprising, e.g., SEQ ID NO: 130.

In some embodiments, the viral genome comprises, from 5′ to 3′: (a) a 5′ ITR, e.g., a 5′ITR comprising the nucleotide sequence of SEQ ID NO: 126 or 130 or variant thereof, (b) a promoter, e.g., a promoter comprising the nucleotide sequence of any one of SEQ ID NOs: 4000-4026 or variant thereof, (c) an exon, e.g., an exon comprising the nucleotide sequence of any one of SEQ ID NOs: 4045-4048 or variant thereof, (d) a nucleotide sequence encoding a modulatory polynucleotide (e.g., a SOD1 targeting RNA agent, e.g., a siRNA duplex), e.g., a modulatory polynucleotide comprising the nucleotide sequence of any one of SEQ ID NOs: 2559-2579 and 5022 or variant thereof, (e) a poly A signal sequence, e.g.,, a poly A signal sequence comprising the nucleotide sequence of SEQ ID NO: 129 or 4027 or variant thereof, and (f) a 3′ITR, e.g., a 3′ITR comprising the nucleotide sequence of SEQ ID NO: 126 or 130 or variant thereof, wherein the variant comprises a sequence differing by no more than 15, 14, 13, 12, 11, 10, 9, 8, 7, 6, 5, 4, 3, 2 or 1 nucleotides, e.g., substitutions, insertions, or deletions, relative to the reference sequence, e.g., a reference sequence, or a sequence with at least 70%, 75%, 80%, 85%, 90%, 95%, 99% or 100% identity, relative to the reference sequence, e.g., wherein the reference sequence is a wild-type sequence.

In some embodiments, the viral genome comprises, from 5′ to 3′: (a) a 5′ ITR, e.g., a 5′ITR comprising the nucleotide sequence of SEQ ID NO: 126 or 130 or variant thereof, (b) a promoter, e.g., a promoter comprising the nucleotide sequence of any one of SEQ ID NOs: 4000-4026 or variant thereof, (c) a first exon, e.g., a first exon comprising the nucleotide sequence of any one of SEQ ID NOs: 4045-4048 or variant thereof, (d) an intron, e.g., an intron comprising the nucleotide sequence of SEQ ID NO: 4044 or variant thereof, (e) a nucleotide sequence encoding a modulatory polynucleotide (e.g., a SOD1 targeting RNA agent, e.g., a siRNA duplex), e.g., a SOD1 targeting polynucleotide comprising the nucleotide sequence of any one of SEQ ID NOs: 2559-2579 and 5022 or variant thereof, (f) a poly A signal sequence, e.g.,, a poly A signal sequence comprising the nucleotide sequence of SEQ ID NO: 129 or 4027 or variant thereof, and (g) a 3′ITR, e.g., a 3′ITR comprising the nucleotide sequence of SEQ ID NO: 126 or 130 or variant thereof, wherein the variant comprises a sequence differing by no more than 15, 14, 13, 12, 11, 10, 9, 8, 7, 6, 5, 4, 3, 2 or 1 nucleotides, e.g., substitutions, insertions, or deletions, relative to the reference sequence, e.g., a reference sequence, or a sequence with at least 70%, 75%, 80%, 85%, 90%, 95%, 99% or 100% identity relative to the reference sequence, e.g., wherein the reference sequence is a wild-type sequence.

In some embodiments, the viral genome comprises, from 5′ to 3′: (a) a 5′ ITR, e.g., a 5′ITR comprising the nucleotide sequence of SEQ ID NO: 126 or 130 or variant thereof, (b) a promoter, e.g., a promoter comprising the nucleotide sequence of any one of SEQ ID NOs: 4000-4026 or variant thereof, (c) a first exon, e.g., a first exon comprising the nucleotide sequence of any one of SEQ ID NOs: 4045-4048 or variant thereof, (d) an intron, e.g., an intron comprising the nucleotide sequence of SEQ ID NO: 4044 or variant thereof, (e) a second exon, e.g., a second exon comprising the nucleotide sequence of any one of SEQ ID NOs: 4045-4048 or variant thereof, optionally wherein the first and second exons are not the same sequence, (f) a nucleotide sequence encoding a modulatory polynucleotide (e.g., a SOD1 targeting RNA agent, e.g., a siRNA duplex), e.g., a SOD1 targeting polynucleotide comprising the nucleotide sequence of any one of SEQ ID NOs: 2559-2579 and 5022 or variant thereof, (g) a poly A signal sequence, e.g.,, a poly A signal sequence comprising the nucleotide sequence of SEQ ID NO: 129 or 4027 or variant thereof, and (h) a 3′ITR, e.g., a 3′ITR comprising the nucleotide sequence of SEQ ID NO: 126 or 130 or variant thereof, wherein the variant comprises a sequence differing by no more than 15, 14, 13, 12, 11, 10, 9, 8, 7, 6, 5, 4, 3, 2 or 1 nucleotides, e.g., substitutions, insertions, or deletions, relative to the reference sequence, e.g., a reference sequence, or a sequence with at least 70%, 75%, 80%, 85%, 90%, 95%, 99% or 100% identity relative to the reference sequence, e.g., wherein the reference sequence is a wild-type sequence.

In some embodiments, the viral genome comprises the nucleotide sequence of any one of SEQ ID NOs: 4028-4041, a nucleotide sequence differing by no more than 15, 14, 13, 12, 11, 10, 9, 8, 7, 6, 5, 4, 3, 2 or 1 nucleotides, e.g., substitutions (e.g., conservative substitutions), insertions, or deletions, relative to any one of SEQ ID NOs: 4028-4041, or a nucleotide sequence with at least 70%, 75%, 80%, 85%, 90%, 95%, 99% or 100% identity to any one of SEQ ID NOs: 4028-4041.

In some embodiments, the AAV particle comprises a 5′ITR sequence which comprises at least 15 (e.g., at least 16, 17, 18, 19, 20, 21, 22, or 23) contiguous nucleotides differing by no more than 3, 2 or 1 nucleotides from the 5′ ITR sequence in Tables 16-18. These differing nucleotides may be contiguous, or alternatively, they may be non-contiguous throughout the sequence. In some embodiments, the differing nucleotides are located at the ends of the sequences. The 5′ITR sequence may have 70-80%, 70-90%, 70-99%, 70-100%, 80-85%, 80-90%, 80-95%, 80-99%, 80-100%, 90-95%, 90-99%, or 90-100% identity to the 5′ ITR sequence in Table 16.

In some embodiments, the AAV particle comprises a Albumin derived filler sequence which comprises at least 15 (e.g., at least 16, 17, 18, 19, 20, 21, 22, or 23) contiguous nucleotides differing by no more than 3, 2 or 1 nucleotides from the Albumin derived filler sequence in Table 16. These differing nucleotides may be contiguous, or alternatively, they may be non-contiguous throughout the sequence. In some embodiments, the differing nucleotides are located at the ends of the sequences. The Albumin derived filler sequence may have 70-80%, 70-90%, 70-99%, 70-100%, 80-85%, 80-90%, 80-95%, 80-99%, 80-100%, 90-95%, 90-99%, or 90-100% identity to the Albumin derived filler sequence in Table 16.

In some embodiments, the AAV particle comprises a lentivirus derived filler sequence which comprises at least 15 (e.g., at least 16, 17, 18, 19, 20, 21, 22, or 23) contiguous nucleotides differing by no more than 3, 2 or 1 nucleotides from SEQ ID NO: 82. These differing nucleotides may be contiguous, or alternatively, they may be non-contiguous throughout the sequence. In some embodiments, the differing nucleotides are located at the ends of the sequences. The lentivirus derived filler sequence may have 70-80%, 70-90%, 70-99%, 70-100%, 80-85%, 80-90%, 80-95%, 80-99%, 80-100%, 90-95%, 90-99%, or 90-100% identity SEQ ID NP: 82.

In some embodiments, the AAV particle comprises a promoter sequence (e.g., an H1 promoter sequence or a promoter sequence in Table 17, 18 or 7, e.g., SEQ ID NO: 128 or 83) which comprises at least 15 (e.g., at least 16, 17, 18, 19, 20, 21, 22, or 23) contiguous nucleotides differing by no more than 7, 6, 5, 4, 3, 2 or 1 nucleotides from the H1 promoter sequence in Table 16 or a promoter sequence in Table 17, 18, or 7. These differing nucleotides may be contiguous, or alternatively, they may be non-contiguous throughout the sequence. In some embodiments, the differing nucleotides are located at the ends of the sequences. The promoter sequence (e.g., an H1 promoter sequence or a promoter sequence in Tables 17, 18 or 6) may have 70-80%, 70-90%, 70-99%, 70-100%, 80-85%, 80-90%, 80-95%, 80-99%, 80-100%, 90-95%, 90-99%, or 90-100% identity to the H1 promoter sequence in Table 16 or a promoter sequence in Tables 17, 18 or 7.

In some embodiments, the AAV particle comprises a first and/or second exon, an intron, or a first exon, intron, and a second exon (“exon-intron-exon cassette”) (e.g., a first and/or second exon, intron, or exon-intron-exon cassette from Tables 17, 18 or 7, e.g., hBG first and/or second exon, intron, or exon-intron-exon cassette) which comprises at least 15 (e.g., at least 16, 17, 18, 19, 20, 21, 22, or 23) contiguous nucleotides differing by no more than 7, 6, 5, 4, 3, 2 or 1 nucleotides from the first and/or second exon, intron, or exon-intron-exon cassette in Tables 17, 18 or 9. These differing nucleotides may be contiguous, or alternatively, they may be non-contiguous throughout the sequence. In some embodiments, the differing nucleotides are located at the ends of the sequences. The first and/or second exon, intron, or exon-intron-exon cassette (e.g., a first and/or second exon, intron, or exon-intron-exon cassette from Tables 17, 18 or 9, e.g., hBG first and/or second exon, intron or exon-intron-exon cassette) may have 70-80%, 70-90%, 70-99%, 70-100%, 80-85%, 80-90%, 80-95%, 80-99%, 80-100%, 90-95%, 90-99%, or 90-100% identity to the first and/or second exon, intron, or exon-intron-exon cassette in Tables 17, 18 or 9.

In some embodiments, the AAV particle comprises a Poly A sequence which comprises at least 15 (e.g., at least 16, 17, 18, 19, 20, 21, 22, or 23) contiguous nucleotides differing by no more than 3, 2 or 1 nucleotides from the polyA sequence in Tables 16-18 or 8. These differing nucleotides may be contiguous, or alternatively, they may be non-contiguous throughout the sequence. In some embodiments, the differing nucleotides are located at the ends of the sequences. The Poly A sequence may have 70-80%, 70-90%, 70-99%, 70-100%, 80-85%, 80-90%, 80-95%, 80-99%, 80-100%, 90-95%, 90-99%, or 90-100% identity to the polyA sequence in Tables 16-18 or 8.

In some embodiments, the AAV particle comprises a 3′ITR sequence which comprises at least 15 (e.g., at least 16, 17, 18, 19, 20, 21, 22, or 23) contiguous nucleotides differing by no more than 3, 2 or 1 nucleotides from the 3′ ITR sequence in Tables 16-18. These differing nucleotides may be contiguous, or alternatively, they may be non-contiguous throughout the sequence. In some embodiments, the differing nucleotides are located at the ends of the sequences. The 3′ITR sequence may have 70-80%, 70-90%, 70-99%, 70-100%, 80-85%, 80-90%, 80-95%, 80-99%, 80-100%, 90-95%, 90-99%, or 90-100% identity to the 3′ITR in Tables 16-18.

AAV particles may be modified to enhance the efficiency of delivery. Such modified AAV particles comprising the nucleic acid sequence encoding the siRNA molecules of the present disclosure can be packaged efficiently and can be used to successfully infect the target cells at high frequency and with minimal toxicity.

In other embodiments, the AAV particle comprising a nucleic acid sequence for encoding siRNA molecules of the present disclosure may be a pseudotyped hybrid or chimeric AAV particle which contains sequences and/or components originating from at least two different AAV serotypes. Pseudotyped AAV particles may be vectors comprising an AAV genome derived from one AAV serotype and a capsid protein derived at least in part from a different AAV serotype. As non-limiting examples, such pseudotyped AAV particles may be vectors comprising an AAV2-derived genome in an AAV1-derived capsid; or vectors comprising an AAV2-derived genome in an AAV6-derived capsid; or vectors comprising an AAV2-derived genome in an AAV4-derived capsid; or an AAV2-derived genome in an AAV9-derived capsid. In like fashion, the present disclosure contemplates any hybrid or chimeric AAV particle.

In other embodiments, AAV particles comprising a nucleic acid sequence encoding the siRNA molecules of the present disclosure may be used to deliver siRNA molecules to the central nervous system (e.g., U.S. Pat. No. 6,180,613; the contents of which is herein incorporated by reference in its entirety).

In some aspects, the AAV particles comprising a nucleic acid sequence encoding the siRNA molecules of the present disclosure may further comprise a modified capsid including peptides from non-viral origin. In other aspects, the AAV particle may contain a CNS specific chimeric capsid to facilitate the delivery of encoded siRNA duplexes into the brain and the spinal cord. For example, an alignment of cap nucleotide sequences from AAV variants exhibiting CNS tropism may be constructed to identify variable region (VR) sequence and structure.

In other embodiments, the siRNA molecules of the present disclosure can be encoded in plasmid vectors, viral vectors (e.g., AAV vectors), genome or other nucleic acid expression vectors for delivery to a cell.

DNA expression plasmids can be used to stably express the siRNA duplexes or dsRNA of the present disclosure in cells and achieve long-term inhibition of target gene.

In one aspect, the sense and antisense strands of a siRNA duplex encoded by a SOD1 targeting polynucleotide are typically linked by a short spacer sequence leading to the expression of a stem-loop structure termed short hairpin RNA (shRNA). The hairpin is recognized and cleaved by Dicer, thus generating mature siRNA molecules.

According to the present disclosure, AAV particles comprising the nucleic acids of the siRNA molecules targeting SOD1 mRNA can further comprise, a capsid protein chosen from VOY101, AAVPHP.B (PHP.B), AAVPHP.N (PHP.N), AAV1, AAV2, AAV5, AAV9, AAV9 K449R, AAVrh10, and variants thereof.

In some embodiments, the siRNA duplexes or dsRNA of the present disclosure when expressed suppress (or degrade) target mRNA (e.g., SOD1). Accordingly, the siRNA duplexes or dsRNA encoded by a SOD1 targeting polynucleotide can be used to substantially inhibit SOD1 gene expression in a cell, for example a motor neuron. In some aspects, the inhibition of SOD1 gene expression refers to an inhibition by at least about 20%, preferably by at least about 30%, 40%, 50%, 60%, 70%, 80%, 85%, 90%, 95% and 100%. Accordingly, the protein product of the targeted gene may be inhibited by at least about 20%, preferably by at least about 30%, 40%, 50%, 60%, 70%, 80%, 85%, 90%, 95% and 100%. The SOD1 gene can be either a wild type gene or a mutated SOD1 gene with at least one mutation. Accordingly, the protein is either wild type protein or a mutated polypeptide with at least one mutation.

### III. Viral Production

### General Viral Production Process

Cells for the production of AAV, e.g., rAAV, particles may comprise, in some embodiments, mammalian cells (such as HEK293 cells) and/or insect cells (such as Sf9 cells).

In various embodiments, AAV production includes processes and methods for producing AAV particles and vectors which can contact a target cell to deliver a payload, e.g. a recombinant viral construct, which includes a nucleotide encoding a payload molecule. In certain embodiments, the viral vectors are adeno-associated viral (AAV) vectors such as recombinant adeno-associated viral (rAAV) vectors. In certain embodiments, the AAV particles are adeno-associated viral (AAV) particles such as recombinant adeno-associated viral (rAAV) particles.

In some embodiments, disclosed herein is a vector comprising a viral genome of the present disclosure. In some embodiments, disclosed herein is a cell comprising a viral genome of the present disclosure. In some embodiments, the cell is a bacterial cell, a mammalian cell (e.g., a HEK293 cell), or an insect cell (e.g., an Sf9 cell).

In some embodiments, disclosed herein is a method of making a viral genome. The method comprising providing a nucleic acid encoding a viral genome described herein and a backbone region suitable for replication of the viral genome in a cell, e.g., a bacterial cell (e.g., wherein the backbone region comprises one or both of a bacterial origin of replication and a selectable marker), and excising the viral genome from the backbone region, e.g., by cleaving the nucleic acid molecule at upstream and downstream of the viral genome. In some embodiments, the viral genome comprising a promoter operably linked to nucleic acid encoding a polynucleotide (e.g., a SOD1 targeting siRNA described herein), will be incorporated into an AAV particle produced in the cell. In some embodiments, the cell is a bacterial cell, a mammalian cell (e.g., a HEK293 cell), or an insect cell (e.g., an Sf9 cell).

In some embodiments, disclosed herein is a method of making a recombinant AAV particle of the present disclosure, the method comprising (i) providing a host cell comprising a viral genome described herein and incubating the host cell under conditions suitable to enclose the viral genome in a capsid protein, e.g., a capsid protein described herein (e.g., a capsid protein listed in Table 2, e.g., a VOY101 capsid protein or functional variant thereof), thereby making the recombinant AAV particle. In some embodiments, the method comprises prior to step (i), introducing a first nucleic acid comprising the viral genome into a cell. In some embodiments, the host cell comprises a second nucleic acid encoding the capsid protein. In some embodiments, the second nucleic acid is introduced into the host cell prior to, concurrently with, or after the first nucleic acid molecule. In some embodiments, the host cell is a bacterial cell, a mammalian cell (e.g., a HEK293 cell), or an insect cell (e.g., an Sf9 cell).

In various embodiments, methods are provided herein of producing AAV particles or vectors by (a) contacting a viral production cell with one or more viral expression constructs encoding at least one AAV capsid protein, and one or more payload constructs encoding a payload molecule, which can be selected from: a transgene, a polynucleotide encoding protein, and a modulatory nucleic acid; (b) culturing the viral production cell under conditions such that at least one AAV particle or vector is produced, and (c) isolating the AAV particle or vector from the production stream.

In these methods, a viral expression construct may encode at least one structural protein and/or at least one non-structural protein. The structural protein may include any of the native or wild type capsid proteins VP1, VP2, and/or VP3, or a chimeric protein thereof. The non-structural protein may include any of the native or wild type Rep78, Rep68, Rep52, and/or Rep40 proteins or a chimeric protein thereof.

In certain embodiments, contacting occurs via transient transfection, viral transduction, and/or electroporation.

In certain embodiments, the viral production cell is selected from a mammalian cell and an insect cell. In certain embodiments, the insect cell includes a Spodoptera frugiperda insect cell. In certain embodiments, the insect cell includes a Sf9 insect cell. In certain embodiments, the insect cell includes a Sf21 insect cell.

The payload construct vector of the present disclosure may include, in various embodiments, at least one inverted terminal repeat (ITR) and may include mammalian DNA.

Also provided are AAV particles and viral vectors produced according to the methods described herein.

In various embodiments, the AAV particles of the present disclosure may be formulated as a pharmaceutical composition with one or more acceptable excipients.

In certain embodiments, an AAV particle or viral vector may be produced by a method described herein.

In certain embodiments, the AAV particles may be produced by contacting a viral production cell (e.g., an insect cell or a mammalian cell) with at least one viral expression construct encoding at least one capsid protein and at least one payload construct vector. The viral production cell may be contacted by transient transfection, viral transduction, and/or electroporation. The payload construct vector may include a payload construct encoding a payload molecule such as, but not limited to, a polynucleotide encoding protein, and a modulatory nucleic acid. The viral production cell can be cultured under conditions such that at least one AAV particle or vector is produced, isolated (e.g., using temperature-induced lysis, mechanical lysis and/or chemical lysis) and/or purified (e.g., using filtration, chromatography, and/or immunoaffinity purification). As a non-limiting example, the payload construct vector may include mammalian DNA.

In certain embodiments, the AAV particles are produced in an insect cell (e.g., Spodoptera frugiperda (Sf9) cell) using a method described herein. As a non-limiting example, the insect cell is contacted using viral transduction which may include baculoviral transduction.

In certain embodiments, the AAV particles are produced in a mammalian cell (e.g., HEK293 cell) using a method described herein. As a non-limiting example, the mammalian cell is contacted using viral transduction which may include multiplasmid transient transfection (such as triple plasmid transient transfection).

In certain embodiments, the AAV particle production method described herein produces greater than 10¹, greater than 10², greater than 10³, greater than 10⁴, or greater than 10⁵ AAV particles in a viral production cell.

In certain embodiments, a process of the present disclosure includes production of viral particles in a viral production cell using a viral production system which includes at least one viral expression construct and at least one payload construct. The at least one viral expression construct and at least one payload construct can be co-transfected (e.g. dual transfection, triple transfection) into a viral production cell. The transfection is completed using standard molecular biology techniques known and routinely performed by a person skilled in the art. The viral production cell provides the cellular machinery necessary for expression of the proteins and other biomaterials necessary for producing the AAV particles, including Rep proteins which replicate the payload construct and Cap proteins which assemble to form a capsid that encloses the replicated payload constructs. The resulting AAV particle is extracted from the viral production cells and processed into a pharmaceutical preparation for administration.

In various embodiments, once administered, an AAV particle disclosed herein may, without being bound by theory, contact a target cell and enter the cell, e.g., in an endosome. The AAV particles, e.g., those released from the endosome, may subsequently contact the nucleus of the target cell to deliver the payload construct. The payload construct, e.g. recombinant viral construct, may be delivered to the nucleus of the target cell wherein the payload molecule encoded by the payload construct may be expressed.

In certain embodiments, the process for production of viral particles utilizes seed cultures of viral production cells that include one or more baculoviruses (e.g., a Baculoviral Expression Vector (BEV) or a baculovirus infected insect cell (BIIC) that has been transfected with a viral expression construct and a payload construct vector). In certain embodiments, the seed cultures are harvested, divided into aliquots and frozen, and may be used at a later time point to initiate an infection of a naïve population of production cells.

In some embodiments, large scale production of AAV particles utilizes a bioreactor. Without being bound by theory, the use of a bioreactor may allow for the precise measurement and/or control of variables that support the growth and activity of viral production cells such as mass, temperature, mixing conditions (impellor RPM or wave oscillation), CO₂ concentration, O₂ concentration, gas sparge rates and volumes, gas overlay rates and volumes, pH, Viable Cell Density (VCD), cell viability, cell diameter, and/or optical density (OD). In certain embodiments, the bioreactor is used for batch production in which the entire culture is harvested at an experimentally determined time point and AAV particles are purified. In some embodiments, the bioreactor is used for continuous production in which a portion of the culture is harvested at an experimentally determined time point for purification of AAV particles, and the remaining culture in the bioreactor is refreshed with additional growth media components.

In various embodiments, AAV viral particles can be extracted from viral production cells in a process which includes cell lysis, clarification, sterilization and purification. Cell lysis includes any process that disrupts the structure of the viral production cell, thereby releasing AAV particles. In certain embodiments, cell lysis may include thermal shock, chemical, or mechanical lysis methods. Clarification can include the gross purification of the mixture of lysed cells, media components, and AAV particles. In certain embodiments, clarification includes centrifugation and/or filtration, including but not limited to depth end, tangential flow, and/or hollow fiber filtration.

In various embodiments, the end result of viral production is a purified collection of AAV particles which include two components: (1) a payload construct (e.g. a recombinant AAV viral genome construct) and (2) a viral capsid.

In certain embodiments, a viral production system or process of the present disclosure includes steps for producing baculovirus infected insect cells (BIICs) using Viral Production Cells (VPC) and plasmid constructs. Viral Production Cells (VPCs) from a Cell Bank (CB) are thawed and expanded to provide a target working volume and VPC concentration. The resulting pool of VPCs is split into a Rep/Cap VPC pool and a Payload VPC pool. One or more Rep/Cap plasmid constructs (viral expression constructs) are processed into Rep/Cap Bacmid polynucleotides and transfected into the Rep/Cap VPC pool. One or more Payload plasmid constructs (payload constructs) are processed into Payload Bacmid polynucleotides and transfected into the Payload VPC pool. The two VPC pools are incubated to produce P1 Rep/Cap Baculoviral Expression Vectors (BEVs) and P1 Payload BEVs. The two BEV pools are expanded into a collection of Plaques, with a single Plaque being selected for Clonal Plaque (CP) Purification (also referred to as Single Plaque Expansion). The process can include a single CP Purification step or can include multiple CP Purification steps either in series or separated by other processing steps. The one-or-more CP Purification steps provide a CP Rep/Cap BEV pool and a CP Payload BEV pool. These two BEV pools can then be stored and used for future production steps, or they can be then transfected into VPCs to produce a Rep/Cap BIIC pool and a Payload BIIC pool.

In certain embodiments, a viral production system or process of the present disclosure includes steps for producing AAV particles using Viral Production Cells (VPC) and baculovirus infected insect cells (BIICs). Viral Production Cells (VPCs) from a Cell Bank (CB) are thawed and expanded to provide a target working volume and VPC concentration. The working volume of Viral Production Cells is seeded into a Production Bioreactor and can be further expanded to a working volume of 200-2000 L with a target VPC concentration for BIIC infection. The working volume of VPCs in the Production Bioreactor is then co-infected with Rep/Cap BIICs and Payload BIICs, with a target VPC: BIIC ratio and a target BIIC: BIIC ratio. VCD infection can also utilize BEVs. The co-infected VPCs are incubated and expanded in the Production Bioreactor to produce a bulk harvest of AAV particles and VPCs.

### Viral Expression Constructs

In various embodiments, the viral production system of the present disclosure includes one or more viral expression constructs that can be transfected/transduced into a viral production cell. In certain embodiments, a viral expression construct or a payload construct of the present disclosure can be a bacmid, also known as a baculovirus plasmid or recombinant baculovirus genome. In certain embodiments, the viral expression includes a protein-coding nucleotide sequence and at least one expression control sequence for expression in a viral production cell. In certain embodiments, the viral expression includes a protein-coding nucleotide sequence operably linked to least one expression control sequence for expression in a viral production cell. In certain embodiments, the viral expression construct contains parvoviral genes under control of one or more promoters. Parvoviral genes can include nucleotide sequences encoding non-structural AAV replication proteins, such as Rep genes which encode Rep52, Rep40, Rep68, or Rep78 proteins. Parvoviral genes can include nucleotide sequences encoding structural AAV proteins, such as Cap genes which encode VP1, VP2, and VP3 proteins.

Viral expression constructs of the present disclosure may include any compound or formulation, biological or chemical, which facilitates transformation, transfection, or transduction of a cell with a nucleic acid. Exemplary biological viral expression constructs include plasmids, linear nucleic acid molecules, and recombinant viruses including baculovirus. Exemplary chemical vectors include lipid complexes. Viral expression constructs are used to incorporate nucleic acid sequences into virus replication cells in accordance with the present disclosure. (O'Reilly, David R., Lois K. Miller, and Verne A. Luckow. Baculovirus expression vectors: a laboratory manual. Oxford University Press, 1994.); Maniatis et al., eds. Molecular Cloning. CSH Laboratory, NY, N.Y. (1982); and, Philiport and Scluber, eds. Liposomes as tools in Basic Research and Industry. CRC Press, Ann Arbor, Mich. (1995), the contents of each of which are herein incorporated by reference in their entirety as related to viral expression constructs and uses thereof.

In certain embodiments, the viral expression construct is an AAV expression construct which includes one or more nucleotide sequences encoding non-structural AAV replication proteins, structural AAV capsid proteins, or a combination thereof.

In certain embodiments, the viral expression construct of the present disclosure may be a plasmid vector. In certain embodiments, the viral expression construct of the present disclosure may be a baculoviral construct.

The present disclosure is not limited by the number of viral expression constructs employed to produce AAV particles or viral vectors. In certain embodiments, one, two, three, four, five, six, or more viral expression constructs can be employed to produce AAV particles in viral production cells in accordance with the present disclosure. In certain embodiments of the present disclosure, a viral expression construct may be used for the production of an AAV particles in insect cells. In certain embodiments, modifications may be made to the wild type AAV sequences of the capsid and/or rep genes, for example to improve attributes of the viral particle, such as increased infectivity or specificity, or to enhance production yields.

In certain embodiments, the viral expression construct may contain a nucleotide sequence which includes start codon region, such as a sequence encoding AAV capsid proteins which include one or more start codon regions. In certain embodiments, the start codon region can be within an expression control sequence. The start codon can be ATG or a non-ATG codon (i.e., a suboptimal start codon where the start codon of the AAV VP1 capsid protein is a non-ATG).

In certain embodiments, the viral expression construct used for AAV production may contain a nucleotide sequence encoding the AAV capsid proteins where the initiation codon of the AAV VP1 capsid protein is a non-ATG, e.g., a suboptimal initiation codon, allowing the expression of a modified ratio of the viral capsid proteins in the production system, to provide improved infectivity of the host cell. In a non-limiting example, a viral construct vector may contain a nucleic acid construct comprising a nucleotide sequence encoding AAV VP1, VP2, and VP3 capsid proteins, wherein the initiation codon for translation of the AAV VP1 capsid protein is CTG, TTG, or GTG, as described in US Patent No. U.S. Pat. No. 8,163,543, the contents of which are herein incorporated by reference in their entirety as related to AAV capsid proteins and the production thereof.

In certain embodiments, the viral expression construct of the present disclosure may be a plasmid vector or a baculoviral construct that encodes the parvoviral rep proteins for expression in insect cells. In certain embodiments, a single coding sequence is used for the Rep78 and Rep52 proteins, wherein start codon for translation of the Rep78 protein is a suboptimal start codon, selected from the group consisting of ACG, TTG, CTG, and GTG, that effects partial exon skipping upon expression in insect cells, as described in U.S. Pat. No. 8,512,981, the contents of which are herein incorporated by reference in their entirety, for example to promote less abundant expression of Rep78 as compared to Rep52, which may promote high vector yields.

In certain embodiments, a VP-coding region encodes one or more AAV capsid proteins of a specific AAV serotype. The AAV serotypes for VP-coding regions can be the same or different. In certain embodiments, a VP-coding region can be codon optimized. In certain embodiments, a VP-coding region or nucleotide sequence can be codon optimized for a mammal cell. In certain embodiments, a VP-coding region or nucleotide sequence can be codon optimized for an insect cell. In certain embodiments, a VP-coding region or nucleotide sequence can be codon optimized for a Spodoptera frugiperda cell. In certain embodiments, a VP-coding region or nucleotide sequence can be codon optimized for Sf9 or Sf21 cell lines.

In certain embodiments, a nucleotide sequence encoding one or more VP capsid proteins can be codon optimized to have a nucleotide homology with the reference nucleotide sequence of less than 100%. In certain embodiments, the nucleotide homology between the codon-optimized VP nucleotide sequence and the reference VP nucleotide sequence is less than 100%, less than 99%, less than 98%, less than 97%, less than 96%, less than 95%, less than 94%, less than 93%, less than 92%, less than 91%, less than 90%, less than 89%, less than 88%, less than 87%, less than 86%, less than 85%, less than 84%, less than 83%, less than 82%, less than 81%, less than 80%, less than 78%, less than 76%, less than 74%, less than 72%, less than 70%, less than 68%, less than 66%, less than 64%, less than 62%, less than 60%, less than 55%, less than 50%, and less than 40%.

In certain embodiments, a viral expression construct or a payload construct of the present disclosure can be a bacmid, also known as a baculovirus plasmid or recombinant baculovirus genome. In certain embodiments, a viral expression construct or a payload construct of the present disclosure (e.g. bacmid) can include a polynucleotide incorporated by homologous recombination (transposon donor/acceptor system) into the bacmid by standard molecular biology techniques known and performed by a person skilled in the art.

In certain embodiments, the polynucleotide incorporated into the bacmid (i.e. polynucleotide insert) can include an expression control sequence operably linked to a protein-coding nucleotide sequence. In certain embodiments, the polynucleotide incorporated into the bacmid can include an expression control sequence which includes a promoter, such as p10 or polh, and which is operably linked to a nucleotide sequence which encodes a structural AAV capsid protein (e.g. VP1, VP2, VP3 or a combination thereof). In certain embodiments, the polynucleotide incorporated into the bacmid can include an expression control sequence which includes a promoter, such as p10 or polh, and which is operably linked to a nucleotide sequence which encodes a non-structural AAV capsid protein (e.g. Rep78, Rep52, or a combination thereof).

The method of the present disclosure is not limited by the use of specific expression control sequences. However, when a certain stoichiometry of VP products are achieved (close to 1:1:10 for VP1, VP2, and VP3, respectively) and also when the levels of Rep52 or Rep40 (also referred to as the p19 Reps) are significantly higher than Rep78 or Rep68 (also referred to as the p5 Reps), improved yields of AAV in production cells (such as insect cells) may be obtained. In certain embodiments, the p5/p19 ratio is below 0.6 more, below 0.4, or below 0.3, but always at least 0.03. These ratios can be measured at the level of the protein or can be implicated from the relative levels of specific mRNAs.

In certain embodiments, AAV particles are produced in viral production cells (such as mammalian or insect cells) wherein all three VP proteins are expressed at a stoichiometry approaching, about or which is: 1:1:10 (VP1:VP2:VP3); 2:2:10 (VP1:VP2:VP3); 2:0:10 (VP1:VP2:VP3); 1-2:0-2:10 (VP1:VP2:VP3); 1-2:1-2:10 (VP1:VP2:VP3); 2-3:0-3:10 (VP1:VP2:VP3); 2-3:2-3:10 (VP1:VP2:VP3); 3:3:10 (VP1:VP2:VP3); 3-5:0-5:10 (VP1:VP2:VP3); or 3-5:3-5:10 (VP1:VP2:VP3).

In certain embodiments, the expression control regions are engineered to produce a VP1:VP2:VP3 ratio selected from the group consisting of: about or exactly 1:0:10; about or exactly 1:1:10; about or exactly 2:1:10; about or exactly 2:1:10; about or exactly 2:2:10; about or exactly 3:0:10; about or exactly 3:1:10; about or exactly 3:2:10; about or exactly 3:3:10; about or exactly 4:0:10; about or exactly 4:1:10; about or exactly 4:2:10; about or exactly 4:3:10; about or exactly 4:4:10; about or exactly 5:5:10; about or exactly 1-2:0-2:10; about or exactly 1-2:1-2:10; about or exactly 1-3:0-3:10; about or exactly 1-3:1-3:10; about or exactly 1-4:0-4:10; about or exactly 1-4:1-4:10; about or exactly 1-5:1-5:10; about or exactly 2-3:0-3:10; about or exactly 2-3:2-3:10; about or exactly 2-4:2-4:10; about or exactly 2-5:2-5:10; about or exactly 3-4:3-4:10; about or exactly 3-5:3-5:10; and about or exactly 4-5:4-5:10.

In certain embodiments of the present disclosure, Rep52 or Rep78 is transcribed from the baculoviral derived polyhedron promoter (polh). Rep52 or Rep78 can also be transcribed from a weaker promoter, for example a deletion mutant of the ie-1 promoter, the Aie-1 promoter, has about 20% of the transcriptional activity of that ie-1 promoter. A promoter substantially homologous to the Aie-1 promoter may be used. In respect to promoters, a homology of at least 50%, 60%, 70%, 80%, 90% or more, is considered to be a substantially homologous promoter.

### Mammalian Cells

Viral production of the present disclosure disclosed herein describes processes and methods for producing AAV particles or viral vector that contacts a target cell to deliver a payload construct, e.g. a recombinant AAV particle or viral construct, which includes a nucleotide encoding a payload molecule. The viral production cell may be selected from any biological organism, including prokaryotic (e.g., bacterial) cells, and eukaryotic cells, including, insect cells, yeast cells and mammalian cells.

In certain embodiments, the AAV particles of the present disclosure may be produced in a viral production cell that includes a mammalian cell. Viral production cells may comprise mammalian cells such as A549, WEH1, 3T3, 10T1/2, BHK, MDCK, COS 1, COS 7, BSC 1, BSC 40, BMT 10, VERO, W138, HeLa, HEK293, HEK293T (293T), Saos, C2C12, L cells, HT1080, Huh7, HepG2, C127, 3T3, CHO, HeLa cells, KB cells, BHK and primary fibroblast, hepatocyte, and myoblast cells derived from mammals. Viral production cells can include cells derived from any mammalian species including, but not limited to, human, monkey, mouse, rat, rabbit, and hamster or cell type, including but not limited to fibroblast, hepatocyte, tumor cell, cell line transformed cell, etc.

AAV viral production cells commonly used for production of recombinant AAV particles include, but is not limited to other mammalian cell lines as described in U.S. Pat. Nos. 6,156,303, 5,387,484, 5,741,683, 5,691,176, 6,428,988 and 5,688,676; U.S. patent application 2002/0081721, and International Patent Publication Nos. WO 00/47757, WO 00/24916, and WO 96/17947, the contents of each of which are herein incorporated by reference in their entireties insofar as they do no conflict with the present disclosure. In certain embodiments, the AAV viral production cells are trans-complementing packaging cell lines that provide functions deleted from a replication-defective helper virus, e.g., HEK293 cells or other Ea trans-complementing cells.

In certain embodiments, the packaging cell line 293-10-3 (ATCC Accession No. PTA-2361) may be used to produce the AAV particles, as described in US Patent No. U.S. Pat. No. 6,281,010, the contents of which are herein incorporated by reference in their entirety as related to the 293-10-3 packaging cell line and uses thereof.

In certain embodiments, of the present disclosure a cell line, such as a HeLA cell line, for trans-complementing E1 deleted adenoviral vectors, which encoding adenovirus Ela and adenovirus E1b under the control of a phosphoglycerate kinase (PGK) promoter can be used for AAV particle production as described in U.S. Pat. No. 6,365,394, the contents of which are incorporated herein by reference in their entirety as related to the HeLa cell line and uses thereof.

In certain embodiments, AAV particles are produced in mammalian cells using a multiplasmid transient transfection method (such as triple plasmid transient transfection). In certain embodiments, the multiplasmid transient transfection method includes transfection of the following three different constructs: (i) a payload construct, (ii) a Rep/Cap construct (parvoviral Rep and parvoviral Cap), and (iii) a helper construct. In certain embodiments, the triple transfection method of the three components of AAV particle production may be utilized to produce small lots of virus for assays including transduction efficiency, target tissue (tropism) evaluation, and stability. In certain embodiments, the triple transfection method of the three components of AAV particle production may be utilized to produce large lots of materials for clinical or commercial applications.

AAV particles to be formulated may be produced by triple transfection or baculovirus mediated virus production, or any other method known in the art. Any suitable permissive or packaging cell known in the art may be employed to produce the vectors. In certain embodiments, trans-complementing packaging cell lines are used that provide functions deleted from a replication-defective helper virus, e.g., 293 cells or other Ela trans-complementing cells.

The gene cassette may contain some or all of the parvovirus (e.g., AAV) cap and rep genes. In certain embodiments, some or all of the cap and rep functions are provided in trans by introducing a packaging vector(s) encoding the capsid and/or Rep proteins into the cell. In certain embodiments, the gene cassette does not encode the capsid or Rep proteins. Alternatively, a packaging cell line is used that is stably transformed to express the cap and/or rep genes.

Recombinant AAV virus particles are, in certain embodiments, produced and purified from culture supernatants according to the procedure as described in US2016/0032254, the contents of which are incorporated by reference in their entirety as related to the production and processing of recombinant AAV virus particles. Production may also involve methods known in the art including those using 293T cells, triple transfection or any suitable production method.

In certain embodiments, mammalian viral production cells (e.g. 293T cells) can be in an adhesion/adherent state (e.g. with calcium phosphate) or a suspension state (e.g. with polyethyleneimine (PEI)). The mammalian viral production cell is transfected with plasmids required for production of AAV, (i.e., AAV rep/cap construct, an adenoviral helper construct, and/or ITR flanked payload construct). In certain embodiments, the transfection process can include optional medium changes (e.g. medium changes for cells in adhesion form, no medium changes for cells in suspension form, medium changes for cells in suspension form if desired). In certain embodiments, the transfection process can include transfection mediums such as DMEM or F17. In certain embodiments, the transfection medium can include serum or can be serum-free (e.g. cells in adhesion state with calcium phosphate and with serum, cells in suspension state with PEI and without serum).

Cells can subsequently be collected by scraping (adherent form) and/or pelleting (suspension form and scraped adherent form) and transferred into a receptacle. Collection steps can be repeated as necessary for full collection of produced cells. Next, cell lysis can be achieved by consecutive freeze-thaw cycles (−80° C. to 37C), chemical lysis (such as adding detergent triton), mechanical lysis, or by allowing the cell culture to degrade after reaching ~0% viability. Cellular debris is removed by centrifugation and/or depth filtration. The samples are quantified for AAV particles by DNase resistant genome titration by DNA qPCR.

AAV particle titers are measured according to genome copy number (genome particles per milliliter). Genome particle concentrations are based on DNA qPCR of the vector DNA as previously reported (Clark et al. (1999) Hum. Gene Ther., 10:1031-1039; Veldwijk et al. (2002) Mol. Ther., 6:272-278, the contents of which are each incorporated by reference in their entireties as related to the measurement of particle concentrations).

### Insect Cells

Viral production of the present disclosure includes processes and methods for producing AAV particles or viral vectors that contact a target cell to deliver a payload construct, e.g., a recombinant viral construct, which includes a nucleotide encoding a payload molecule. In certain embodiments, the AAV particles or viral vectors of the present disclosure may be produced in a viral production cell that includes an insect cell.

Growing conditions for insect cells in culture, and production of heterologous products in insect cells in culture are well-known in the art, see U.S. Pat. No. 6,204,059, the contents of which are herein incorporated by reference in their entirety as related to the growth and use of insect cells in viral production.

Any insect cell which allows for replication of parvovirus and which can be maintained in culture can be used in accordance with the present disclosure. AAV viral production cells commonly used for production of recombinant AAV particles include, but is not limited to, Spodoptera frugiperda, including, but not limited to the Sf9 or Sf21 cell lines, Drosophila cell lines, or mosquito cell lines, such as Aedes albopictus derived cell lines. Use of insect cells for expression of heterologous proteins is well documented, as are methods of introducing nucleic acids, such as vectors, e.g., insect-cell compatible vectors, into such cells and methods of maintaining such cells in culture. See, for example, Methods in Molecular Biology, ed. Richard, Humana Press, NJ (1995); O'Reilly et al., Baculovirus Expression Vectors, A Laboratory Manual, Oxford Univ. Press (1994); Samulski et al., J. Vir.63:3822-8 (1989); Kajigaya et al., Proc. Nat'l. Acad. Sci. USA 88:4646-50 (1991); Ruffing et al., J. Vir. 66:6922-30 (1992); Kimbauer et al., Vir.219:37-44 (1996); Zhao et al., Vir.272:382-93 (2000); and Samulski et al., U.S. Pat. No. 6,204,059, the contents of each of which are herein incorporated by reference in their entirety as related to the use of insect cells in viral production.

In some embodiments, the AAV particles are made using the methods described in WO2015/191508, the contents of which are herein incorporated by reference in their entirety insofar as they do not conflict with the present disclosure.

In certain embodiments, insect host cell systems, in combination with baculoviral systems (e.g., as described by Luckow et al., Bio/Technology 6:47 (1988)) may be used. In certain embodiments, an expression system for preparing chimeric peptide is Trichoplusia ni, Tn 5B1-4 insect cells/baculoviral system, which can be used for high levels of proteins, as described in U.S. Pat. No. 6,660,521, the contents of which are herein incorporated by reference in their entirety as related to the production of viral particles.

Expansion, culturing, transfection, infection and storage of insect cells can be carried out in any cell culture media, cell transfection media or storage media known in the art, including Hyclone™ SFX-Insect™ Cell Culture Media, Expression System ESF AF™ Insect Cell Culture Medium, ThermoFisher Sf-900II™ media, ThermoFisher Sf-900III™ media, or ThermoFisher Grace's Insect Media. Insect cell mixtures of the present disclosure can also include any of the formulation additives or elements described in the present disclosure, including (but not limited to) salts, acids, bases, buffers, surfactants (such as Poloxamer 188/Pluronic F-68), and other known culture media elements. Formulation additives can be incorporated gradually or as “spikes” (incorporation of large volumes in a short time).

### Baculovirus-Production Systems

In certain embodiments, processes of the present disclosure can include production of AAV particles or viral vectors in a baculoviral system using a viral expression construct and a payload construct vector. In certain embodiments, the baculoviral system includes Baculovirus expression vectors (BEVs) and/or baculovirus infected insect cells (BIICs). In certain embodiments, a viral expression construct or a payload construct of the present disclosure can be a bacmid, also known as a baculovirus plasmid or recombinant baculovirus genome. In certain embodiments, a viral expression construct or a payload construct of the present disclosure can be polynucleotide incorporated by homologous recombination (transposon donor/acceptor system) into a bacmid by standard molecular biology techniques known and performed by a person skilled in the art. Transfection of separate viral replication cell populations produces two or more groups (e.g. two, three) of baculoviruses (BEVs), one or more group which can include the viral expression construct (Expression BEV), and one or more group which can include the payload construct (Payload BEV). The baculoviruses may be used to infect a viral production cell for production of AAV particles or viral vector.

In certain embodiments, the process includes transfection of a single viral replication cell population to produce a single baculovirus (BEV) group which includes both the viral expression construct and the payload construct. These baculoviruses may be used to infect a viral production cell for production of AAV particles or viral vector.

In certain embodiments, BEVs are produced using a Bacmid Transfection agent, such as Promega FuGENE® HD, WFI water, or ThermoFisher Cellfectin® II Reagent. In certain embodiments, BEVs are produced and expanded in viral production cells, such as an insect cell.

In certain embodiments, the method utilizes seed cultures of viral production cells that include one or more BEVs, including baculovirus infected insect cells (BIICs). The seed BIICs have been transfected/transduced/infected with an Expression BEV which includes a viral expression construct, and also a Payload BEV which includes a payload construct. In certain embodiments, the seed cultures are harvested, divided into aliquots and frozen, and may be used at a later time to initiate transfection/transduction/infection of a naïve population of production cells. In certain embodiments, a bank of seed BIICs is stored at −80° C. or in LN2 vapor.

Baculoviruses are made of several essential proteins which are essential for the function and replication of the Baculovirus, such as replication proteins, envelope proteins and capsid proteins. The Baculovirus genome thus includes several essential-gene nucleotide sequences encoding the essential proteins. As a non-limiting example, the genome can include an essential-gene region which includes an essential-gene nucleotide sequence encoding an essential protein for the Baculovirus construct. The essential protein can include: GP64 baculovirus envelope protein, VP39 baculovirus capsid protein, or other similar essential proteins for the Baculovirus construct.

Baculovirus expression vectors (BEV) for producing AAV particles in insect cells, including but not limited to Spodoptera frugiperda (Sf9) cells, provide high titers of viral vector product. Recombinant baculovirus encoding the viral expression construct and payload construct initiates a productive infection of viral vector replicating cells. Infectious baculovirus particles released from the primary infection secondarily infect additional cells in the culture, exponentially infecting the entire cell culture population in a number of infection cycles that is a function of the initial multiplicity of infection, see Urabe, M. et al. J Virol. 2006 February;80 (4): 1874-85, the contents of which are herein incorporated by reference in their entirety as related to the production and use of BEVs and viral particles.

Production of AAV particles with baculovirus in an insect cell system may address known baculovirus genetic and physical instability.

In certain embodiments, the production system of the present disclosure addresses baculovirus instability over multiple passages by utilizing a titerless infected-cells preservation and scale-up system. Small scale seed cultures of viral producing cells are transfected with viral expression constructs encoding the structural and/or non-structural components of the AAV particles. Baculovirus-infected viral producing cells are harvested into aliquots that may be cryopreserved in liquid nitrogen; the aliquots retain viability and infectivity for infection of large scale viral producing cell culture. Wasilko D J et al. Protein Expr Purif. 2009 June;65 (2): 122-32, the contents of which are herein incorporated by reference in their entirety as related to the production and use of BEVs and viral particles.

A genetically stable baculovirus may be used to produce a source of the one or more of the components for producing AAV particles in invertebrate cells. In certain embodiments, defective baculovirus expression vectors may be maintained episomally in insect cells. In such embodiments, the corresponding bacmid vector is engineered with replication control elements, including but not limited to promoters, enhancers, and/or cell-cycle regulated replication elements.

In certain embodiments, stable viral producing cells permissive for baculovirus infection are engineered with at least one stable integrated copy of any of the elements necessary for AAV replication and vector production including, but not limited to, the entire AAV genome, Rep and Cap genes, Rep genes, Cap genes, each Rep protein as a separate transcription cassette, each VP protein as a separate transcription cassette, the AAP (assembly activation protein), or at least one of the baculovirus helper genes with native or non-native promoters.

In some embodiments, the AAV particle of the present disclosure may be produced in insect cells (e.g., Sf9 cells).

In some embodiments, the AAV particle of the present disclosure may be produced using triple transfection.

In some embodiments, the AAV particle of the present disclosure may be produced in mammalian cells.

In some embodiments, the AAV particle of the present disclosure may be produced by triple transfection in mammalian cells.

In some embodiments, the AAV particle of the present disclosure may be produced by triple transfection in HEK293 cells.

The AAV viral genomes encoding a SOD1 targeting siRNA described herein may be useful in the fields of human disease, veterinary applications and a variety of in vivo and in vitro settings. The AAV particles of the present disclosure may be useful in the field of medicine for the treatment, prophylaxis, palliation, or amelioration of neurological or neuromuscular diseases and/or disorders. In some embodiments, the AAV particles of the disclosure are used for the prevention and/or treatment of SOD1-related disorders.

Various embodiments of the disclosure herein provide a pharmaceutical composition comprising the AAV particle described herein and a pharmaceutically acceptable excipient.

Various embodiments of the disclosure herein provide a method of treating a subject in need thereof comprising administering to the subject a therapeutically effective amount of the pharmaceutical composition described herein.

Certain embodiments of the method provide that the subject is treated by a route of administration of the pharmaceutical composition selected from the group consisting of: intravenous, intracerebroventricular, intraparenchymal, intrathecal, subpial, and intramuscular, or a combination thereof. Certain embodiments of the method provide that the subject is treated for SOD1-related disorders. In one aspect of the method, a pathological feature of the SOD1-related disorder is alleviated and/or the progression of the SOD1-related disorder is halted, slowed, ameliorated, or reversed.

Various embodiments of the disclosure herein describe a method of increasing the level of SOD1 targeting siRNAs in the central nervous system of a subject in need thereof comprising administering to said subject via infusion, an effective amount of the pharmaceutical composition described herein.

Also described herein are compositions, methods, processes, kits and devices for the design, preparation, manufacture and/or formulation of AAV particles. In some embodiments, payloads, such as but not limited to payloads comprising SOD1 targeting siRNAs, may be encoded by payload constructs or contained within plasmids or vectors or recombinant adeno-associated viruses (AAVs).

The present disclosure also provides administration and/or delivery methods for vectors and viral particles, e.g., AAV particles, for the treatment or amelioration of SOD1-related disorders. Such methods may involve gene replacement or gene activation. Such outcomes are achieved by utilizing the methods and compositions taught herein.

IV. Introduction into Cells

To ensure the chemical and biological stability of siRNA duplexes, it is important to deliver polynucleotides encoding the siRNAs inside the target cells. The polynucleotides of the present disclosure may be introduced into cells using any of a variety of approaches.

In some embodiments, the polynucleotide of the present disclosure is introduced into a cell by contacting the cell with the polynucleotide. In some embodiments, the polynucleotide is introduced into a cell by contacting the cell with a composition comprising the polynucleotide and a lipophilic carrier. In other embodiments, the polynucleotide is introduced into a cell by transfecting or infecting the cell with a vector comprising nucleic acid sequences capable of producing the siRNA duplex when transcribed in the cell.

In some embodiments, the siRNA duplex is introduced into a cell by injecting into the cell a vector comprising nucleic acid sequences capable of producing the siRNA duplex when transcribed in the cell.

In other embodiments, the polynucleotides of the present disclosure may be delivered into cells by electroporation (e.g. U.S. Patent Publication No. 20050014264; the content of which is herein incorporated by reference in its entirety).

In addition, the siRNA molecules inserted into viral vectors (e.g. AAV vectors) may be delivered into cells by viral infection. These viral vectors are engineered and optimized to facilitate the entry of siRNA molecule into cells that are not readily amendable to transfection. Also, some synthetic viral vectors possess an ability to integrate the shRNA into the cell genome, thereby leading to stable siRNA expression and long-term knockdown of a target gene. In this manner, viral vectors are engineered as vehicles for specific delivery while lacking the deleterious replication and/or integration features found in wild-type virus.

In some embodiments, the cells may include, but are not limited to, cells of mammalian origin, cells of human origins, embryonic stem cells, induced pluripotent stem cells, neural stem cells, and neural progenitor cells.

### V. Pharmaceutical Compositions and Formulation

The present disclosure additionally provides a method for treating SOD1-related disorders and disorders related to deficiencies in the function or expression of SOD1 in a mammalian subject, including a human subject, comprising administering to the subject any of the AAV polynucleotides or AAV genomes described herein (i.e., “vector genomes,” “viral genomes,” or “VGs”) or administering to the subject a particle comprising said AAV polynucleotide or AAV genome, or administering to the subject any of the described compositions, including pharmaceutical compositions.

As used herein the term “composition” comprises an AAV polynucleotide or AAV genome or AAV particle and at least one excipient.

As used herein the term “pharmaceutical composition” comprises an AAV polynucleotide or AAV genome or AAV particle and one or more pharmaceutically acceptable excipients.

Although the descriptions of pharmaceutical compositions, e.g., AAV comprising a payload encoding a SOD1 targeting siRNA to be delivered, provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

In some embodiments, compositions are administered to humans, human patients, or subjects.

In some embodiments, the AAV particle formulations described herein may contain a nucleic acid encoding at least one payload. In some embodiments, the formulations may contain a nucleic acid encoding 1, 2, 3, 4, or 5 payloads. In some embodiments, the formulation may contain a nucleic acid encoding a payload construct encoding proteins selected from categories such as, but not limited to, human proteins, veterinary proteins, bacterial proteins, biological proteins, antibodies, immunogenic proteins, therapeutic peptides and proteins, secreted proteins, plasma membrane proteins, cytoplasmic proteins, cytoskeletal proteins, intracellular membrane bound proteins, nuclear proteins, proteins associated with human disease, and/or proteins associated with non-human diseases. In some embodiments, the formulation contains at least three payload constructs encoding proteins. Certain embodiments provide that at least one of the payloads is a SOD1 targeting polynucleotide (e.g., a SOD1 targeting RNAi, e.g., a siRNA duplex for inhibiting expression of SOD1) described herein, or a variant thereof.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

### VI. Formulations

Formulations of the AAV pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered.

For example, the composition may comprise between 0.1% and 99% (w/w) of the active ingredient. By way of example, the composition may comprise between 0.1% and 100%, e.g., between 0.5% and 50%, between 1-30%, between 5-80%, or at least 80% (w/w) active ingredient.

In some embodiments, the formulations described herein may contain at least one SOD1 targeting polynucleotide (e.g., a SOD1 targeting RNAi, e.g., a siRNA duplex for inhibiting expression of SOD1). As a non-limiting example, the formulations may contain 1, 2, 3, 4 or 5 polynucleotide that target the SOD1 gene at different sites.

The AAV particles of the disclosure can be formulated using one or more excipients to: (1) increase stability; (2) increase cell transfection or transduction; (3) permit the sustained or delayed release; (4) alter the biodistribution (e.g., target the viral particle to specific tissues or cell types); (5) increase the translation of encoded protein in vivo; (6) alter the release profile of encoded protein in vivo and/or (7) allow for regulatable expression of the payload.

Formulations of the present disclosure can include, without limitation, saline, lipidoids, liposomes, lipid nanoparticles, polymers, lipoplexes, core-shell nanoparticles, peptides, proteins, cells transfected with viral vectors (e.g., for transplantation into a subject), nanoparticle mimics and combinations thereof. Further, the viral vectors of the present disclosure may be formulated using self-assembled nucleic acid nanoparticles.

In some embodiments, the viral vectors encoding the SOD1 targeting polynucleotides may be formulated to optimize baricity and/or osmolality. In some embodiments, the baricity and/or osmolality of the formulation may be optimized to ensure optimal drug distribution in the central nervous system or a region or component of the central nervous system.

In some embodiments, the AAV particles of the disclosure may be formulated in PBS with 0.001% of pluronic acid (F-68) at a pH of about 7.0.

In some embodiments, the AAV particles of the disclosure may be formulated in PBS, in combination with an ethylene oxide/propylene oxide copolymer (also known as pluronic or poloxamer).

In some embodiments, the AAV particles of the disclosure may be formulated in PBS with 0.001% pluronic acid (F-68) (poloxamer 188) at a pH of about 7.0.

In some embodiments, the AAV particles of the disclosure may be formulated in PBS with 0.001% pluronic acid (F-68) (poloxamer 188) at a pH of about 7.3.

In some embodiments, the AAV particles of the disclosure may be formulated in PBS with 0.001% pluronic acid (F-68) (poloxamer 188) at a pH of about 7.4.

In some embodiments, the AAV particles of the disclosure may be formulated in a solution comprising sodium chloride, sodium phosphate and an ethylene oxide/propylene oxide copolymer.

In some embodiments, the AAV particles of the disclosure may be formulated in a solution comprising sodium chloride, sodium phosphate dibasic, potassium chloride, potassium phosphate monobasic, and poloxamer 188/pluronic acid (F-68).

In some embodiments, the AAV particles of the disclosure may be formulated in a solution comprising 192 mM sodium chloride, 10 mM sodium phosphate (dibasic), 2.7 mM potassium chloride, 2 mM potassium phosphate (monobasic) and 0.001% pluronic F-68 (v/v), at pH 7.4. This formulation is referred to as Formulation 1 in the present disclosure.

In some embodiments, the AAV particles of the disclosure may be formulated in a solution comprising about 192 mM sodium chloride, about 10 mM sodium phosphate dibasic and about 0.001% poloxamer 188, at a pH of about 7.3. The concentration of sodium chloride in the final solution may be 150 mM-200 mM. As non-limiting examples, the concentration of sodium chloride in the final solution may be 150 mM, 160 mM, 170 mM, 180 mM, 190 mM or 200 mM. The concentration of sodium phosphate dibasic in the final solution may be 1 mM-50 mM. As non-limiting examples, the concentration of sodium phosphate dibasic in the final solution may be 1 mM, 2 mM, 3 mM, 4 mM, 5 mM, 6 mM, 7 mM, 8 mM, 9 mM, 10 mM, 15 mM, 20 mM, 25 mM, 30 mM, 40 mM, or 50 mM. The concentration of poloxamer 188 (pluronic acid (F-68)) may be 0.0001%-1%. As non-limiting examples, the concentration of poloxamer 188 (pluronic acid (F-68)) may be 0.0001%, 0.0005%, 0.001%, 0.005%, 0.01%, 0.05%, 0.1%, 0.5%, or 1%. The final solution may have a pH of 6.8-7.7. Non-limiting examples for the pH of the final solution include a pH of 6.8, 6.9, 7.0, 7.1, 7.2, 7.3, 7.4, 7.5, 7.6, or 7.7.

In some embodiments, the AAV particles of the disclosure may be formulated in a solution comprising about 1.05% sodium chloride, about 0.212% sodium phosphate dibasic, heptahydrate, about 0.025% sodium phosphate monobasic, monohydrate, and 0.001% poloxamer 188, at a pH of about 7.4. As a non-limiting example, the concentration of AAV particle in this formulated solution may be about 0.001%. The concentration of sodium chloride in the final solution may be 0.1-2.0%, with non-limiting examples of 0.1%, 0.25%, 0.5%, 0.75%, 0.95%, 0.96%, 0.97%, 0.98%, 0.99%, 1.00%, 1.01%, 1.02%, 1.03%, 1.04%, 1.05%, 1.06%, 1.07%, 1.08%, 1.09%, 1.10%, 1.25%, 1.5%, 1.75%, or 2%. The concentration of sodium phosphate dibasic in the final solution may be 0.100-0.300% with non-limiting examples including 0.100%, 0.125%, 0.150%, 0.175%, 0.200%, 0.210%, 0.211%, 0.212%, 0.213%, 0.214%, 0.215%, 0.225%, 0.250%, 0.275%, 0.300%. The concentration of sodium phosphate monobasic in the final solution may be 0.010-0.050%, with non-limiting examples of 0.010%, 0.015%, 0.020%, 0.021%, 0.022%, 0.023%, 0.024%, 0.025%, 0.026%, 0.027%, 0.028%, 0.029%, 0.030%, 0.035%, 0.040%, 0.045%, or 0.050%. The concentration of poloxamer 188 (pluronic acid (F-68)) may be 0.0001%-1%. As non-limiting examples, the concentration of poloxamer 188 (pluronic acid (F-68)) may be 0.0001%, 0.0005%, 0.001%, 0.005%, 0.01%, 0.05%, 0.1%, 0.5%, or 1%. The final solution may have a pH of 6.8-7.7. Non-limiting examples for the pH of the final solution include a pH of 6.8, 6.9, 7.0, 7.1, 7.2, 7.3, 7.4, 7.5, 7.6, or 7.7.

### Excipients

The formulations of the disclosure can include one or more excipients, each in an amount that together increases the stability of the AAV particle, increases cell transfection or transduction by the viral particle, increases the expression of viral particle encoded protein, and/or alters the release profile of AAV particle encoded proteins. In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Excipients, which, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, MD, 2006; the contents of which are herein incorporated by reference in their entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

### Inactive Ingredients

In some embodiments, AAV formulations may comprise at least one excipient which is an inactive ingredient. As used herein, the term “inactive ingredient” refers to one or more agents that do not contribute to the activity of the pharmaceutical composition included in formulations. In some embodiments, all, none, or some of the inactive ingredients which may be used in the formulations of the present disclosure may be approved by the US Food and Drug Administration (FDA).

Formulations of AAV particles disclosed herein may include cations or anions. In some embodiments, the formulations include metal cations such as, but not limited to, Zn²⁺, Ca²⁺, Cu²⁺, Mg⁺, or combinations thereof. In some embodiments, formulations may include polymers or polynucleotides complexed with a metal cation (see, e.g., U.S. Pat. Nos. 6,265,389 and 6,555,525, the contents of each of which are herein incorporated by reference in their entirety).

### VII. Uses and Applications

Provided in the present disclosure are methods for introducing the SOD1 targeting polynucleotides described herein into cells, the method comprising introducing into said cells any of the polynucleotides in an amount sufficient for degradation of target SOD1 mRNA to occur. In some aspects, the cells may be stem cells, neurons such as motor neurons, muscle cells and glial cells such as astrocytes.

Described here are methods for delivering AAV particles to the spinal cord, for the treatment of disorders associated with the spinal cord, such as, but not limited to motor neuron disease (e.g., ALS). In certain embodiments, these methods result in trans-synaptic transmission.

Disclosed herein are also methods for treating ALS associated with abnormal SOD1 function in a subject in need of treatment. The method optionally comprises administering to the subject a therapeutically effective amount of a composition comprising or encoding at least one siRNA duplex targeting SOD1 gene. Said siRNA duplex will silence SOD1 gene expression and inhibit SOD1 protein production and reduce one or more symptoms of ALS in the subject such that ALS is therapeutically treated.

In some embodiment, administration to a subject will reduce the expression of mutant and/or wild-type SOD1 in the central nervous system (e.g., brain tissue, spinal cord, and/or motor neurons) and/or and the reduction of expression of the mutant and/or wild-type SOD1 will reduce the effects of ALS in a subject.

In some embodiment, the administration is to a subject who is in the early stages of ALS. Early stage symptoms include, but are not limited to, muscles which are weak and soft or stiff, tight and spastic, cramping and twitching (fasciculations) of muscles, loss of muscle bulk (atrophy), fatigue, poor balance, slurred words, weak grip, and/or tripping when walking. The symptoms may be limited to a single body region or a mild symptom may affect more than one region. As a non-limiting example, administration of the AAV viral particle may reduce the severity and/or occurrence of the symptoms of ALS.

In some embodiment, the administration is to a subject who is in the middle stages of ALS. The middle stage of ALS includes, but is not limited to, more widespread muscle symptoms as compared to the early stage, some muscles are paralyzed while others are weakened or unaffected, continued muscle twitchings (fasciculations), unused muscles may cause contractures where the joints become rigid, painful and sometimes deformed, weakness in swallowing muscles may cause choking and greater difficulty eating and managing saliva, weakness in breathing muscles can cause respiratory insufficiency which can be prominent when lying down, and/or a subject may have bouts of uncontrolled and inappropriate laughing or crying (pseudobulbar affect). As a non-limiting example, administration of the AAV particles may reduce the severity and/or occurrence of the symptoms of ALS.

In one embodiment, the administration is to a subject who is in the late stages of ALS. The late stage of ALS includes, but is not limited to, voluntary muscles which are mostly paralyzed, the muscles that help move air in and out of the lungs are severely compromised, mobility is extremely limited, poor respiration may cause fatigue, fuzzy thinking, headaches and susceptibility to infection or diseases (e.g., pneumonia), speech is difficult and eating or drinking by mouth may not be possible.

In some embodiments, the AAV particles may be used to treat a subject with ALS who has a C9orf72 mutation, TDP-43 mutations, and/or FUS mutations.

In some embodiments, the SOD1 targeting polynucleotide of the present disclosure or the composition comprising or encoding the polynucleotides is administered to the central nervous system of the subject. In other embodiments, the siRNA duplex of the present disclosure or the composition comprising it is administered to the muscles of the subject

In particular, the SOD1 targeting polynucleotides may be delivered into specific types of targeted cells, including motor neurons; glial cells including oligodendrocyte, astrocyte and microglia; and/or other cells surrounding neurons such as T cells. Studies in human ALS patients and animal SOD1 ALS model implicated that glial cells play an early role in the dysfunction and death of ALS neurons. Normal SOD1 in the surrounding, protective glial cells can prevent the motor neurons from dying even though mutant SOD1 is present in motor neurons (e.g., reviewed by Philips and Rothstein, Exp. Neurol., 2014 May 22. pii: S0014-4886 (14) 00157-5; the content of which is incorporated herein by reference in its entirety).

In some specific embodiments, at least one siRNA duplex targeting SOD1 gene used as a therapy for ALS is inserted in an AAV viral genome.

In some embodiments, the present composition is administered as a single therapeutic or combination therapeutics for the treatment of ALS.

The viral vectors comprising or encoding siRNA duplexes targeting SOD1 gene may be used in combination with one or more other therapeutic, agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent.

Therapeutic agents that may be used in combination with the SOD1 targeting polynucleotides of the present disclosure can be small molecule compounds which are antioxidants, anti-inflammatory agents, anti-apoptosis agents, calcium regulators, antiglutamatergic agents, structural protein inhibitors, and compounds involved in metal ion regulation.

Compounds used in combination for treating ALS may include, but are not limited to, agents that reduce oxidative stress, such as free-radical scavengers, or Radicava (edaravone), antiglutamatergic agents: Riluzole, Topiramate, Talampanel, Lamotrigine, Dextromethorphan, Gabapentin and AMPA antagonist; Anti-apoptosis agents: Minocycline, Sodium phenylbutyrate and Arimoclomol; Anti-inflammatory agent: ganglioside, Celecoxib, Cyclosporine, Azathioprine, Cyclophosphamide, Plasmaphoresis, Glatiramer acetate and thalidomide; Ceftriaxone (Berry et al., Plos One, 2013, 8 (4)); Beat-lactam antibiotics; Pramipexole (a dopamine agonist) (Wang et al., Amyotrophic Lateral Scler., 2008, 9 (1), 50-58); Nimesulide in U.S. Patent Publication No. 20060074991; Diazoxide disclosed in U.S. Patent Publication No. 20130143873); pyrazolone derivatives disclosed in US Patent Publication No. 20080161378; free radical scavengers that inhibit oxidative stress-induced cell death, such as bromocriptine (US. Patent Publication No. 20110105517); phenyl carbamate compounds discussed in PCT Patent Publication No. 2013100571; neuroprotective compounds disclosed in U.S. Pat. Nos. 6,933,310 and 8,399,514 and US Patent Publication Nos. 20110237907 and 20140038927; and glycopeptides taught in U.S. Patent Publication No. 20070185012; the content of each of which is incorporated herein by reference in their entirety.

Therapeutic agents that may be used in combination therapy with the siRNA duplexes targeting SOD1 gene of the present disclosure may be hormones or variants that can protect neuron loss, such as adrenocorticotropic hormone (ACTH) or fragments thereof (e.g., U.S. Patent Publication No. 20130259875); Estrogen (e.g., U.S. Pat. Nos. 6,334,998 and 6,592,845); the content of each of which is incorporated herein by reference in their entirety.

Neurotrophic factors may be used in combination therapy with the siRNA duplexes targeting SOD1 gene of the present disclosure for treating ALS. Generally, a neurotrophic factor is defined as a substance that promotes survival, growth, differentiation, proliferation and/or maturation of a neuron, or stimulates increased activity of a neuron. In some embodiments, the present methods further comprise delivery of one or more trophic factors into the subject in need of treatment. Trophic factors may include, but are not limited to, IGF-I, GDNF, BDNF, CTNF, VEGF, Colivelin, Xaliproden, Thyrotrophin-releasing hormone and ADNF, and variants thereof.

In one aspect, the AAV particle comprising at least one siRNA duplex targeting SOD1 gene may be co-administered with AAV particle expressing neurotrophic factors such as AAV-IGF-I (Vincent et al., Neuromolecular medicine, 2004, 6, 79-85; the content of which is incorporated herein by reference in its entirety) and AAV-GDNF (Wang et al., J Neurosci., 2002, 22, 6920-6928; the content of which is incorporated herein by reference in its entirety).

### VIII. Dosing and Administration

### Administration

In some aspects, the present disclosure provides administration and/or delivery methods for vectors and viral particles, e.g., AAV particles, a SOD1 targeting polynucleotide or a variant thereof, for the prevention, treatment, or amelioration of diseases or disorders of the CNS. For example, administration of the AAV particles prevents, treats, or ameliorates SOD1-related disorders. Thus, robust widespread SOD1 targeting siRNA distribution throughout the CNS and periphery is desired for maximal efficacy. Particular target tissues for administration or delivery include CNS tissues, more specifically, brain tissue.

In some aspects, the present disclosure relates to a method of delivering an exogenous siRNA duplex for inhibiting expression of SOD1 to a subject, comprising administering an effective amount of the pharmaceutical composition or the AAV particle described herein to a subject, thereby delivering the exogenous siRNA duplex to the subject.

In specific embodiments, compositions including AAV particles comprising at least one SOD1 targeting polynucleotide may be administered in a way which allows them to enter the central nervous system and penetrate into motor neurons.

The AAV particles of the present disclosure may be administered by any route which results in a therapeutically effective outcome.

In some embodiments, AAV particles of the present disclosure are administered so as to be delivered to a target cell or tissue. Delivery to a target cell results in SOD1 targeting siRNA expression. A target cell may be any cell in which it is considered desirable to increase SOD1 targeting siRNA expression levels. A target cell may be a CNS cell. Non-limiting examples of such cells and/or tissues include, dorsal root ganglia and dorsal columns, proprioceptive sensory neurons, Clark's column, gracile and cuneate nuclei, cerebellar dentate nucleus, corticospinal tracts and the cells comprising the same, Betz cells, and cells of the heart.

In some embodiments, compositions may be administered in a way that allows them to cross the blood-brain barrier, vascular barrier, or other epithelial barrier.

In some embodiments, delivery of SOD1 targeting siRNA by adeno-associated virus (AAV) particles to cells of the central nervous system (e.g., parenchyma) comprises infusion into cerebrospinal fluid (CSF). CSF is produced by specialized ependymal cells that comprise the choroid plexus located in the ventricles of the brain. CSF produced within the brain then circulates and surrounds the central nervous system including the brain and spinal cord. CSF continually circulates around the central nervous system, including the ventricles of the brain and subarachnoid space that surrounds both the brain and spinal cord, while maintaining a homeostatic balance of production and reabsorption into the vascular system. The entire volume of CSF is replaced approximately four to six times per day or approximately once every four hours, though values for individuals may vary.

In some embodiments, the AAV particles may be delivered by systemic delivery. In some embodiments, the systemic delivery may be by intravascular administration. In some embodiments, the systemic delivery may be by intravenous (IV) administration.

In some embodiments, the AAV particles may be delivered by intravenous delivery.

In some embodiments, the AAV particle is administered to the subject via focused ultrasound (FUS), e.g., coupled with the intravenous administration of microbubbles (FUS-MB), or MRI-guided FUS coupled with intravenous administration, e.g., as described in Terstappen et al. (Nat Rev Drug Discovery, https://doi.org/10.1038/s41573-021-00139-y (2021)), Burgess et al. (Expert Rev Neurother. 15 (5): 477-491 (2015)), and/or Hsu et al. (PLOS One 8 (2): 1-8), the contents of which are incorporated herein by reference in its entirety.

In some embodiments, the AAV particles may be delivered by injection into the CSF pathway. Non-limiting examples of delivery to the CSF pathway include intrathecal and intracerebroventricular administration.

In some embodiments, the AAV particles may be delivered by thalamic delivery.

In some embodiments, the AAV particles may be delivered by intracerebral delivery.

In some embodiments, the AAV particles may be delivered by intracardiac delivery.

In some embodiments, the AAV particles may be delivered by intracranial delivery. (See,

e.g., U.S. Pat. No. 8,119,611; the content of which is incorporated herein by reference in its entirety).

In some embodiments, the AAV particles may be delivered by intra cisterna magna (ICM) delivery (i.e., the space around and below the cerebellum via the opening between the skull and the top of the spine). In certain embodiments, the pharmaceutical compositions of the present disclosure may be administered to the cisterna magna in a therapeutically effective amount to transduce spinal cord motor neurons and/or astrocytes. In some embodiments, the AAV particles may be delivered via infusion or injection to the cisterna magna. The administration to the cisterna magna may occur via a bolus injection. Depending on the severity of the symptoms and the responsiveness of the subject to the therapy, the bolus injection may be administered once per week, once per month, once every two months, once every three months, once every 6 months or annually. The administration to the cisterna magna may occur via a catheter or canula. In other embodiments, the administration is achieved by use of an infusion pump. The administration may occur continually over a period of at least several days.

In some embodiments, the AAV particles may be delivered by direct (intraparenchymal) injection into an organ (e.g., CNS (brain or spinal cord)). In some embodiments, the intraparenchymal delivery may be to any region of the brain or CNS. In some embodiments, the AAV particles may be administered by intraparenchymal injection to the CNS, the brain and/or the spinal cord. As a non-limiting example, the AAV particles of the present disclosure may be administered to a subject by intraparenchymal injection. In certain embodiments, the intraparenchymal injection may be a spinal intraparenchymal injection, wherein the pharmaceutical compositions may be administered directly to the tissue of the spinal cord. In certain embodiments, the intraparenchymal injection may be a CNS (central nervous system) intraparenchymal injection wherein the pharmaceutical compositions may be administered directly to the tissue of the CNS.

In some embodiments, the AAV particles may be administered by intraparenchymal injection and intrathecal injection. In certain embodiments, the pharmaceutical compositions of the present disclosure may be administered by intraparenchymal injection and intrastriatal injection.

In some embodiments, the AAV particles may be delivered by intrastriatal injection.

In some embodiments, the AAV particles may be delivered into the putamen.

In some embodiments, the AAV particles may be delivered into the spinal cord.

In some embodiments, the AAV particles of the present disclosure may be administered to the ventricles of the brain.

In some embodiments, the AAV particles of the present disclosure may be administered to the ventricles of the brain by intracerebroventricular delivery.

In some embodiments, the AAV particles of the present disclosure may be administered by intramuscular delivery. Rizvanov et al. demonstrated for the first time that siRNA molecules, targeting mutant human SOD1 mRNA, is taken up by the sciatic nerve, retrogradely transported to the perikarya of motor neurons, and inhibits mutant SOD1 mRNA in SOD1^{G93A} transgenic ALS mice (Rizvanov A A et al., Exp. Brain Res., 2009, 195 (1), 1-4; the content of which is incorporated herein by reference in its entirety). Another study also demonstrated that muscle delivery of AAV expressing small hairpin RNAs (shRNAs) against the mutant SOD1 gene, led to significant mutant SOD1 knockdown in the muscle as well as innervating motor neurons (Towne C et al., Mol Ther., 2011; 19 (2): 274-283; the content of which is incorporated herein by reference in its entirety).

In some embodiments, AAV particles that express siRNA duplexes of the present disclosure may be administered to a subject by peripheral injections and/or intranasal delivery. It was disclosed in the art that the peripheral administration of AAV particles for siRNA duplexes can be transported to the central nervous system, for example, to the motor neurons (e.g., U.S. Patent Publication Nos. 20100240739; and 20100130594; the content of each of which is incorporated herein by reference in their entirety).

In some embodiments, the AAV particles of the present disclosure are administered by more than one route described above. As a non-limiting example, the AAV particles may be administered by intravenous delivery and thalamic delivery.

In some embodiments, the AAV particles of the present disclosure are administered by more than one route described above. As a non-limiting example, the AAV particles may be administered by intravenous delivery and intracerebral delivery.

In some embodiments, the AAV particles of the present disclosure are administered by more than one route described above. As a non-limiting example, the AAV particles may be administered by intravenous delivery and intracranial delivery.

In some embodiments, the AAV particles of the present disclosure are administered by more than one route described above. In some embodiments, the AAV particles of the present disclosure may be delivered by intrathecal and intracerebroventricular administration.

In some embodiments, the AAV particles may be delivered to a subject to preserve neurons. The neurons may be primary and/or secondary sensory neurons. In some embodiments, AAV particles are delivered to dorsal root ganglia and/or neurons thereof. In some embodiments, AAV particles are delivered to motor neurons.

In some embodiments, the AAV particles may be delivered to a subject to improve and/or correct mitochondrial dysfunction.

In some embodiments, administration of the AAV particles may preserve and/or correct function in the sensory pathways.

In some embodiments, the AAV particles may be delivered via intravenous (IV), intracerebroventricular (ICV), intraparenchymal, and/or intrathecal (IT) infusion and the therapeutic agent may also be delivered to a subject via intramuscular (IM) limb infusion in order to deliver the therapeutic agent to the skeletal muscle. Delivery of AAVs by intravascular limb infusion is described by Gruntman and Flotte, Human Gene Therapy Clinical Development, 2015, 26 (3), 159-164, the contents of which are herein incorporated by reference in their entirety.

In some embodiments, delivery of viral vector pharmaceutical compositions in accordance with the present disclosure to cells of the central nervous system (e.g., parenchyma) comprises a rate of delivery defined by VG/hour=mL/hour*VG/mL, wherein VG is viral genomes, VG/mL is composition concentration, and mL/hour is rate of infusion.

In some embodiments, delivery of AAV particle pharmaceutical compositions in accordance with the present disclosure to cells of the central nervous system (e.g., parenchyma) comprises infusion of up to 1 mL. In some embodiments, delivery of viral vector pharmaceutical compositions in accordance with the present disclosure to cells of the central nervous system (e.g., parenchyma) may comprise infusions of 0.0001, 0.0002, 0.001, 0.002, 0.003, 0.004, 0.005, 0.008, 0.010, 0.015, 0.020, 0.025, 0.030, 0.040, 0.050, 0.060, 0.070, 0.080, 0.090, 0.1, 0.2, 0.3, 0.4, 0.5, 0.6, 0.7, 0.8, or 0.9 mL.

In certain embodiments, the AAV particle described herein is administered via intraparenchymal (IPa) infusion at any level of the spinal cord, at a single or at multiple sites, at a volume of more than 1 μL. In certain embodiments, a volume of 1 μL-100 μL is administered.

The spinal cord is situated within the spine. The spine consists of a series of vertebral segments. There are 7 cervical (C1-C₇), 12 thoracic (T1-T12), 5 lumbar (L1-L5), and 5 sacral (S1-S5) vertebral segments. Intraparenchymal injection or infusion into the spinal cord of AAV particles described herein may occur at one or multiple of these vertebral segments. For example, intraparenchymal injection or infusion into the spinal cord of AAV particles described herein may occur at 1, 2, 3, 4, 5, or more than 5 sites. The intraparenchymal injection or infusion sites may be at one or more regions independently selected from the cervical spinal cord, the thoracic spinal cord, the lumbar spinal cord, and the sacral spinal cord. In some embodiments, AAV particles described herein are administered via intraparenchymal (IPa) infusion at two sites into the spinal cord.

In some embodiments, the AAV particle described herein may be administered via intraparenchymal (IPa) infusion to one or more sites (e.g., 2, 3, 4 or 5 sites) selected from C1, C2, C3, C4, C5, C6, and C7. In some embodiments, the AAV particle described herein may be administered via intraparenchymal (IPa) infusion to one or more sites (e.g., 2, 3, 4 or 5 sites) selected from T1, T2, T3, T4, T5, T6, T7, T8, T9, T10, T11, and T12. In some embodiments, the AAV particle described herein may be administered via intraparenchymal (IPa) infusion to one or more sites (e.g., 2, 3, 4 or 5 sites) selected from L1, L2, L3, L4, and L5. In some embodiments, the AAV particle described herein may be administered via intraparenchymal (IPa) infusion to one or more sites (e.g., 2, 3, 4 or 5 sites) selected from S1, S2, S3, S4, and S5.

In some embodiments, the AAV particle described herein may be administered via intraparenchymal (IPa) infusion at one or more sites (e.g., 2, 3, 4 or 5 sites) selected from C1, C2, C3, C4, C5, C6, C7, T1, T2, T3, T4, T5, T6, T7, T8, T9, T10, T11, T12, L1, L2, L3, L4, L5, S1, S2, S3, S4, and S5. As a non-limiting example, the administration is at two sites and may include one site from the cervical spinal cord region (e.g., C1-C₇) and one site from the thoracic spinal cord region (e.g., T1-T12). As a non-limiting example, the administration is at two sites and may include one site from the thoracic spinal cord region (e.g., T1-T12) and one site from the lumbar spinal cord region (e.g., L1-L5).

In certain embodiments, the AAV particle described herein encoding siRNA molecules may be administered via intraparenchymal (IPa) infusion at two sites. The AAV particles may be delivered at the same or different volume for both sites. The AAV particles may be delivered at the same or different volumes for both sites. The AAV particles may be delivered at the same or different infusion rates for both sites.

In some embodiments, IPa infusions (e.g., spinal cord) may result in delivery of the pharmaceutical compositions (i.e., AAV particles) along the extent of the rostral-caudal axis of the spinal cord. In some embodiments, IPa infusions (e.g., spinal cord) yield a rostrocaudal gradient of AAV particle transmission. In some embodiments, IPa infusions (e.g., spinal cord) result in delivery of the pharmaceutical compositions to regions distal to the injection site. While not wishing to be bound by theory, AAV particles of the disclosure may travel the length of the rostral caudal axis of the spinal cord subsequent to IPa infusion at a particular site. In other words, the AAV particles may not confined to the immediate vicinity of the injection site. As a non-limiting example, the AAV particles may be transported by a trans-synaptic (across the synapse) mechanism. This trans-synaptic mechanism may follow a tract or channel present along the rostral-caudal axis of the spinal cord. Additional methods relating to IPa administration are known in the art, including for example U.S. Patent Application Publication US20210254103, which is hereby incorporated by reference in its entirety. Details regarding the effect of SOD1 siRNAs in vitro and in vivo, as well as intraparenchymal administration of SOD1 siRNAs packaged in AAVs, are described in PCT Patent Application Publication WO2020010042, which is hereby incorporated by reference in its entirety, see, e.g., pp 154-175.

In some embodiments, delivery of AAV particle pharmaceutical compositions in accordance with the present disclosure to cells of the central nervous system (e.g., parenchyma) comprises infusion of between about 1 mL to about 120 mL. In some embodiments, delivery of viral vector pharmaceutical compositions in accordance with the present disclosure to cells of the central nervous system (e.g., parenchyma) may comprise an infusion of 0.1, 1, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 mL. In some embodiments delivery of AAV particles to cells of the central nervous system (e.g., parenchyma) comprises infusion of at least 3 mL. In some embodiments, delivery of AAV particles to cells of the central nervous system (e.g., parenchyma) consists of infusion of 3 mL. In some embodiments, delivery of AAV particles to cells of the central nervous system (e.g., parenchyma) comprises infusion of at least 10 mL. In some embodiments, delivery of AAV particles to cells of the central nervous system (e.g., parenchyma) consists of infusion of 10 mL.

In some embodiments, the volume of the AAV particle pharmaceutical composition delivered to the cells of the central nervous system (e.g., parenchyma) of a subject is 2 μl, 20 μl, 50 μl, 80 μl, 100 μl, 200 μl, 300 μl, 400 μl, 500 μl, 600 μl, 700 μl, 800 μl, 900 μl, 1000 μl, 1100 μl, 1200 μl, 1300 μl, 1400 μl, 1500 μl, 1600 μl, 1700 μl, 1800 μl, 1900 μl, 2000 μl, or more than 2000 μl.

In some embodiments, the volume of the AAV particle pharmaceutical composition delivered to a region in both hemispheres of a subject brain is 2 μl, 20 μl, 50 μl, 80 μl, 100 μl, 200 μl, 300 μl, 400 μl, 500 μl, 600 μl, 700 μl, 800 μl, 900 μl, 1000 μl, 1100 μl, 1200 μl, 1300 μl, 1400 μl, 1500 μl, 1600 μl, 1700 μl, 1800 μl, 1900 μl, 2000 μl, or more than 2000 μl.

In certain embodiments, AAV particle or viral vector pharmaceutical compositions in accordance with the present disclosure may be administered at about 10 to about 600 μl/site, about 50 to about 500 μl/site, about 100 to about 400 μl/site, about 120 to about 300 μl/site, about 140 to about 200 μl/site, or about 160 μl/site.

In some embodiments, the total volume delivered to a subject may be split between one or more administration sites e.g., 1, 2, 3, 4, 5, or more than 5 sites. In some embodiments, the total volume is split between administration to the left and right hemisphere.

### Delivery of AAV Particles

In some embodiments, the AAV particles or pharmaceutical compositions of the present disclosure may be administered or delivered using the methods for treatment of disease described in U.S. Pat. No. 8,999,948, or International Publication No. WO2014178863, the contents of which are herein incorporated by reference in their entirety.

In some embodiments, the AAV particles or pharmaceutical compositions of the present disclosure may be administered or delivered using the methods for delivering gene therapy in Alzheimer's Disease or other neurodegenerative conditions as described in U.S. application No. 20150126590, the contents of which are herein incorporated by reference in their entirety.

In some embodiments, the AAV particles or pharmaceutical compositions of the present disclosure may be administered or delivered using the methods for delivery of a CNS gene therapy as described in U.S. Pat. Nos. 6,436,708, and 8,946,152, and International Publication No. WO2015168666, the contents of which are herein incorporated by reference in their entirety.

In some embodiments, the AAV particles of the present disclosure may be administered or delivered using the methods for the delivery of AAV virions described in European Patent Application No. EP1857552, the contents of which are herein incorporated by reference in their entirety.

In some embodiments, the AAV particle or pharmaceutical compositions of the present disclosure may be administered or delivered using the methods for delivering proteins using AAV particles described in European Patent Application No. EP2678433, the contents of which are herein incorporated by reference in their entirety.

In some embodiments, the viral vector encoding a SOD1 targeting siRNA may be administered or delivered using the methods for delivering DNA molecules using AAV particles described in US Patent No. U.S. Pat. No. 5,858,351, the contents of which are herein incorporated by reference in their entirety.

In some embodiments, the AAV particle or pharmaceutical compositions of the present disclosure may be administered or delivered using the methods for delivering DNA to the bloodstream described in US Patent No. U.S. Pat. No. 6,211,163, the contents of which are herein incorporated by reference in their entirety.

In some embodiments, the viral vector encoding a SOD1 targeting siRNA may be administered or delivered using the methods for delivering AAV virions described in US Patent No. U.S. Pat. No. 6,325,998, the contents of which are herein incorporated by reference in their entirety.

In some embodiments, the viral vector encoding a SOD1 targeting siRNA may be administered or delivered using the methods for delivering DNA to muscle cells described in US Patent No. U.S. Pat. No. 6,335,011, the contents of which are herein incorporated by reference in their entirety.

In some embodiments, the viral vector encoding a SOD1 targeting siRNA may be administered or delivered using the methods for delivering DNA to muscle cells and tissues described in US Patent No. U.S. Pat. No. 6,610,290, the contents of which are herein incorporated by reference in their entirety.

In some embodiments, the viral vector encoding a SOD1 targeting siRNA may be administered or delivered using the methods for delivering DNA to muscle cells described in US Patent No. U.S. Pat. No. 7,704,492, the contents of which are herein incorporated by reference in their entirety.

In some embodiments, the viral vector encoding a SOD1 targeting siRNA may be administered or delivered using the methods for delivering a payload to skeletal muscles described in US Patent No. U.S. Pat. No. 7,112,321, the contents of which are herein incorporated by reference in their entirety.

In some embodiments, the AAV particle or pharmaceutical compositions of the present disclosure may be administered or delivered using the methods for delivering a payload to the central nervous system described in US Patent No. U.S. Pat. No. 7,588,757, the contents of which are herein incorporated by reference in their entirety.

In some embodiments, the AAV particle or pharmaceutical compositions of the present disclosure may be administered or delivered using the methods for delivering a payload described in US Patent No. U.S. Pat. No. 8,283,151, the contents of which are herein incorporated by reference in their entirety.

In some embodiments, the AAV particle or pharmaceutical compositions of the present disclosure may be administered or delivered using the methods for delivering a payload for the treatment of Alzheimer disease described in US Patent No. U.S. Pat. No. 8,318,687, the contents of which are herein incorporated by reference in their entirety.

In some embodiments, the AAV particle or pharmaceutical compositions of the present disclosure may be administered or delivered using the methods for delivering a payload described in International Patent Publication No. WO2012144446, the contents of which are herein incorporated by reference in their entirety.

In some embodiments, the AAV particle or pharmaceutical compositions of the present disclosure may be administered or delivered using the methods for delivering a payload using a glutamic acid decarboxylase (GAD) delivery vector described in International Patent Publication No. WO2001089583, the contents of which are herein incorporated by reference in their entirety.

In some embodiments, the AAV particle or pharmaceutical compositions of the present disclosure may be administered or delivered using the methods for delivering a payload to neural cells described in International Patent Publication No. WO2012057363, the contents of which are herein incorporated by reference in their entirety.

In some embodiments, the AAV particle or pharmaceutical compositions of the present disclosure may be administered or delivered using the methods for delivering a payload described in International Patent Publication No. WO2001096587, the contents of which are herein incorporated by reference in their entirety.

In some embodiments, the AAV particle or pharmaceutical compositions of the present disclosure may be administered or delivered using the methods for delivering a payload to muscle tissue described in International Patent Publication No. WO2002014487, the contents of which are herein incorporated by reference in their entirety.

In some embodiments, a catheter may be used to administer the AAV particles. In certain embodiments, the catheter or cannula may be located at more than one site in the spine for multi-site delivery. The viral particles encoding may be delivered in a continuous and/or bolus infusion. Each site of delivery may be a different dosing regimen or the same dosing regimen may be used for each site of delivery. In some embodiments, the sites of delivery may be in the cervical and the lumbar region. In some embodiments, the sites of delivery may be in the cervical region. In some embodiments, the sites of delivery may be in the lumbar region.

In some embodiments, a subject may be analyzed for spinal anatomy and pathology prior to delivery of the AAV particles described herein. As a non-limiting example, a subject with scoliosis may have a different dosing regimen and/or catheter location compared to a subject without scoliosis.

In some embodiments, the delivery method and duration is chosen to provide broad transduction in the spinal cord. In some embodiments, intrathecal delivery is used to provide broad transduction along the rostral-caudal length of the spinal cord. In some embodiments, multi-site infusions provide a more uniform transduction along the rostral-caudal length of the spinal cord.

### Delivery to Cells

In some aspects, the present disclosure provides a method of delivering to a cell or tissue any of the above-described AAV particles, comprising contacting the cell or tissue with said AAV particle or contacting the cell or tissue with a formulation comprising said AAV particle, or contacting the cell or tissue with any of the described compositions, including pharmaceutical compositions. The method of delivering the AAV particle to a cell or tissue can be accomplished in vitro, ex vivo, or in vivo.

### Delivery to Subjects

In some aspects, the present disclosure additionally provides a method of delivering to a subject, including a mammalian subject, any of the above-described AAV particles comprising administering to the subject said AAV particle, or administering to the subject a formulation comprising said AAV particle, or administering to the subject any of the described compositions, including pharmaceutical compositions.

In some embodiments, the AAV particles may be delivered to bypass anatomical blockages such as, but not limited to the blood brain barrier.

In some embodiments, the AAV particles may be formulated and delivered to a subject by a route which increases the speed of drug effect as compared to oral delivery.

In some embodiments, the AAV particles may be delivered by a method to provide uniform transduction of the spinal cord and dorsal root ganglion (DRG). In some embodiments, the AAV particles may be delivered using intrathecal infusion.

In some embodiments, a subject may be administered the AAV particles described herein using a bolus infusion. As used herein, a “bolus infusion” means a single and rapid infusion of a substance or composition.

In some embodiments, the AAV particles encoding a SOD1 targeting siRNA may be delivered in a continuous and/or bolus infusion. Each site of delivery may be a different dosing regimen or the same dosing regimen may be used for each site of delivery. As a non-limiting example, the sites of delivery may be in the cervical and the lumbar region. As another non-limiting example, the sites of delivery may be in the cervical region. As another non-limiting example, the sites of delivery may be in the lumbar region.

In some embodiments, the AAV particles may be delivered to a subject via a single route administration.

In some embodiments, the AAV particles may be delivered to a subject via a multi-site route of administration. For example, a subject may be administered the AAV particles at 2, 3, 4, 5, or more than 5 sites.

In some embodiments, a subject may be administered the AAV particles described herein using sustained delivery over a period of minutes, hours or days. The infusion rate may be changed depending on the subject, distribution, formulation or another delivery parameter known to those in the art.

In some embodiments, if continuous delivery (continuous infusion) of the AAV particles is used, the continuous infusion may be for 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or more than 24 hours.

In some embodiments, the intracranial pressure may be evaluated prior to administration. The route, volume, AAV particle concentration, infusion duration and/or vector titer may be optimized based on the intracranial pressure of a subject.

In some embodiments, the AAV particles may be delivered by systemic delivery. In some embodiments, the systemic delivery may be by intravascular administration.

In some embodiments, the AAV particles may be delivered by injection into the CSF pathway. Non-limiting examples of delivery to the CSF pathway include intrathecal and intracerebroventricular administration.

In some embodiments, the AAV particles may be delivered by direct (intraparenchymal) injection into the substance of an organ, e.g., one or more regions of the brain.

In some embodiments, the AAV particles may be delivered by subpial injection into the spinal cord. For example, subjects may be placed into a spinal immobilization apparatus. A dorsal laminectomy may be performed to expose the spinal cord. Guiding tubes and XYZ manipulators may be used to assist catheter placement. Subpial catheters may be placed into the subpial space by advancing the catheter from the guiding tube and AAV particles may be injected through the catheter (Miyanohara et al., Mol Ther Methods Clin Dev. 2016; 3:16046). In some cases, the AAV particles may be injected into the cervical subpial space. In some cases, the AAV particles may be injected into the thoracic subpial space.

In some embodiments, the AAV particles may be delivered by direct injection to the CNS of a subject. In some embodiments, direct injection is intracerebral injection, intraparenchymal injection, intrathecal injection, intra-cisterna magna injection, or any combination thereof. In some embodiments, direct injection to the CNS of a subject comprises convection enhanced delivery (CED). In some embodiments, administration comprises peripheral injection. In some embodiments, peripheral injection is intravenous injection.

In some embodiments, the AAV particles may be delivered to a subject in order to decrease SOD1 activity in the caudate-putamen, thalamus, superior colliculus, cortex, and/or corpus callosum as compared to endogenous levels. In some embodiments, the AAV particles may be delivered to a subject in order to decrease SOD1 activity in the motor and sensory cranial nerves, as well as neurons of the cortex, the hippocampus, and amygdala as compared to endogenous levels. The decrease may be 0.1× to 5×, 0.5× to 5×, 1× to 5×, 2× to 5×, 3× to 5×, 4× to 5×, 0.1× to 4×, 0.5× to 4×, 1× to 4×, 2× to 4×, 3× to 4×, 0.1× to 3×, 0.5× to 3×, 1× to 3×, 2× to 3×, 0.1× to 2×, 0.5× to 2×, 0.1× to 1×, 0.5× to 1×, 0.1× to 0.5×, 1× to 2×, 0.1×, 0.2×, 0.3×, 0.4×, 0.5×, 0.6×, 0.7×, 0.8×, 0.9×, 1.0×, 1.1×, 1.2×, 1.3×, 1.4×, 1.5×, 1.6×, 1.7×, 1.8×, 1.9×, 2.0×, 2.1×, 2.2×, 2.3×, 2.4×, 2.5×, 2.6×, 2.7×, 2.8×, 2.9×, 3.0×, 3.1×, 3.2×, 3.3×, 3.4×, 3.5×, 3.6×, 3.7×, 3.8×, 3.9×, 4.0×, 4.1×, 4.2×, 4.3×, 4.4×, 4.5×, 4.6×, 4.7×, 4.8×, 4.9× or more than 5× as compared to endogenous levels.

In some embodiments, the AAV particles may be delivered to a subject in order to decrease SOD1 activity levels in the caudate, putamen, thalamus, superior colliculus, cortex, and/or corpus callosum by transducing cells in these CNS regions. In some embodiments, the AAV particles may be delivered to a subject in order to transducing cells in the motor and sensory cranial nerves, as well as neurons of the cortex, the hippocampus, and amygdala by transducing cells in these CNS regions. Transduction may also be referred to as the amount of cells that are positive for the SOD1 targeting siRNA. The transduction may be greater than or equal to 1%, 5%, 10%, 15%, 20%, 25%, 30%, 35%, 40%, 45%, 50%, 55%, 60%, 65%, 70%, 75%, 80%, 85%, 90%, 95%, or 99% of cells in these CNS regions.

In some embodiments, delivery of AAV particles comprising a viral genome encoding the SOD1 targeting siRNA described herein to motor neurons (e.g., motor neurons or ventral horn motor neurons); glial cells including oligodendrocyte, astrocyte and microglia; and/or other cells surrounding neurons such as T cells, will lead to a decrease in the activity of SOD1. The decreased activity of SOD1 may lead to improved survival and function of various cell types in these CNS regions and subsequent improvement of SOD1-related disorder symptoms.

In particular embodiments, the AAV particles may be delivered to a subject in order to establish widespread distribution of the SOD1 targeting siRNA throughout the nervous system by administering the AAV particles to the thalamus of the subject.

In some embodiments, the composition of the present disclosure for treating ALS is administered to the subject in need intravenously, intramuscularly, subcutaneously, intraperitoneally, intrathecally, intrathalamically, intraparenchymally (CNS, brain, and/or spinal cord) and/or intraventricularly, allowing the siRNA duplexes or vectors comprising the siRNA duplexes to pass through one or both the blood-brain barrier and the blood spinal cord barrier. In some aspects, the method includes administering (e.g., intraparenchymally administering, intraventricularly administering and/or intrathecally administering) directly to the central nervous system (CNS) of a subject (using, e.g., an infusion pump and/or a delivery scaffold) a therapeutically effective amount of a composition comprising at least one siRNA duplex targeting SOD1 gene or AAV particles comprising at least one siRNA duplex targeting SOD1 gene, silencing/suppressing SOD1 gene expression, and reducing one or more symptoms of ALS in the subject such that ALS is therapeutically treated.

In some embodiments, the composition of the present disclosure for treating ALS is administered to the subject in need intraparenchymally (CNS, brain, and/or spinal cord), allowing the siRNA duplexes or vectors comprising the siRNA duplexes to pass through one or both the blood-brain barrier and the blood spinal cord barrier.

In certain aspects, the symptoms of ALS including motor neuron degeneration, muscle weakness, muscle atrophy, the stiffness of muscle, difficulty in breathing, slurred speech, fasciculation development, frontotemporal dementia and/or premature death are improved in the subject treated. In other aspects, the composition of the present disclosure is applied to one or both of the brain and the spinal cord. In other aspects, one or both of muscle coordination and muscle function are improved. In other aspects, the survival of the subject is prolonged.

### Dosing

In some aspects, the present disclosure provides methods comprising administering viral vectors and their payloads in accordance with the disclosure to a subject in need thereof. Viral vector pharmaceutical, imaging, diagnostic, or prophylactic compositions thereof, may be administered to a subject using any amount and any route of administration effective for preventing, treating, diagnosing, or imaging a disease, disorder, and/or condition (e.g., a disease, disorder, and/or condition associated with SOD1). In some embodiments, the disease, disorder, and/or condition is SOD1-related disorders. The exact amount required will vary from subject to subject, depending on the species, age, and general condition of the subject, the severity of the disease, the particular composition, its mode of administration, its mode of activity, and the like. Compositions in accordance with the disclosure are typically formulated in unit dosage form for ease of administration and uniformity of dosage. It will be understood, however, that the total daily usage of the compositions of the present disclosure may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex, and diet of the patient; the time of administration, route of administration, and rate of excretion of the specific peptide(s) employed; the duration of the treatment; drugs used in combination or coincidental with the specific compound employed; and like factors well known in the medical arts.

In certain embodiments, AAV particle pharmaceutical compositions in accordance with the present disclosure may be administered at dosage levels sufficient to deliver SOD1 targeting siRNAs from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, prophylactic, or imaging effect. It will be understood that the above dosing concentrations may be converted to VG or viral genomes per kg or into total viral genomes administered by one of skill in the art.

In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. As used herein, a “split dose” is the division of single unit dose or total daily dose into two or more doses, e.g., two or more administrations of the single unit dose. As used herein, a “single unit dose” is a dose of any therapeutic composition administered in one dose/at one time/single route/single point of contact, i.e., single administration event. In some embodiments, a single unit dose is provided as a discrete dosage form (e.g., a tablet, capsule, patch, loaded syringe, vial, etc.). As used herein, a “total daily dose” is an amount given or prescribed in 24-hour period. It may be administered as a single unit dose. The viral particles may be formulated in buffer only or in a formulation described herein.

A pharmaceutical composition described herein can be formulated into a dosage form described herein, such as a topical, intranasal, pulmonary, intratracheal, or injectable (e.g., intravenous, intraocular, intravitreal, intramuscular, intracardiac, intraperitoneal, and/or subcutaneous).

In some embodiments, delivery of the AAV particles described herein results in minimal serious adverse events (SAEs) as a result of the delivery of the AAV particles.

In some embodiments, delivery of AAV particle pharmaceutical compositions in accordance with the present disclosure to cells of the central nervous system (e.g., parenchyma) may comprise a total concentration between about 1×10⁶ VG/mL and about 1×10¹⁶ VG/mL. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 1.6×10¹¹, 1.8×10¹¹, 2×10¹¹, 3×10¹¹, 4×10¹¹, 5×10¹¹, 5.5×10¹¹, 6×10¹¹, 7×10¹¹, 8×10¹¹, 9×10¹¹, 0.8×10¹², 0.83×10¹², 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.1×10¹², 2.2×10¹², 2.3×10¹², 2.4×10¹², 2.5×10¹², 2.6×10¹², 2.7×10¹², 2.8×10¹², 2.9×10¹², 3×10¹², 3.1×10¹², 3.2×10¹², 3.3×10¹², 3.4×10¹², 3.5×10¹², 3.6×10¹², 3.7×10¹², 3.8×10¹², 3.9×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 8×10¹², 9×10¹², 1×10¹³, 2×10¹³, 2.3×10¹³, 3×10^{13 4}×10¹³, 5×10¹³, 6×10¹³, 7×10¹³, 8×10¹³, 9×10¹³, 1×10^{14, 1.9}×10^{14, 2}×10^{14, 3}×10^{14, 4}×10^{14, 5}×10^{14, 6}×10^{14, 7}×10^{14, 8}×10^{14, 9}×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/mL.

In some embodiments, delivery of AAV particle pharmaceutical compositions in accordance with the present disclosure to cells of the central nervous system (e.g., parenchyma) may comprise a total concentration per subject between about 1×10⁶ VG and about 1×10¹⁶ VG. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 1.6×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 4×10¹¹, 4.6×10¹¹, 5×10¹¹, 6×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.3×10¹², 3×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 8×10¹², 8.1×10¹², 8.2×10¹², 8.3×10¹², 8.4×10¹², 8.5×10¹², 8.6×10¹², 8.7×10¹², 8.8×10¹², 8.9×10¹², 9×10¹², 1×10¹³, 2×10¹³, 3×10¹³, 4×10¹³, 5×10¹³, 6×10¹³, 7×10¹³, 8×10¹³, 9×10¹³, 1×10^{14, 2}×10¹⁴, 3×10^{14, 4}×10^{14, 5}×10^{14, 6}×10^{14, 7}×10^{14, 8}×10^{14, 9}×10^{14, 1}×10¹⁵, 2×10¹⁵, 3×10¹⁵, 4×10¹⁵, 5×10^{15 6}×10¹⁵, 7×10¹⁵, 8×10¹⁵, 9×10¹⁵, or 1×10¹⁶ VG/subject.

In some embodiments, delivery of AAV particles to cells of the central nervous system (e.g., parenchyma) may comprise a total dose between about 1×10⁶ VG and about 1×10¹⁶ VG. In some embodiments, delivery may comprise a total dose of about 1×10⁶, 2×10⁶, 3×10⁶, 4×10⁶, 5×10⁶, 6×10⁶, 7×10⁶, 8×10⁶, 9×10⁶, 1× 107, 2× 107, 3×10⁷, 4×10⁷, 5×10⁷, 6×10⁷, 7×10⁷, 8× 107, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 1.9×10¹⁰, 2×10¹⁰, 3×10¹⁰, 3.73×10¹⁰, 4×10¹⁰, 5×10¹⁰, 6×10¹⁰, 7×10¹⁰, 8×10¹⁰, 9×10¹⁰, 1×10¹¹, 2×10¹¹, 2.5×10¹¹, 3× 1011, 4×10¹¹, 5×10¹¹, 6×10¹¹, 7×10¹¹, 8×10¹¹, 9×10¹¹, 1×10¹², 2×10¹², 3×10¹², 4×10¹², 5× 1012, 6×10¹², 7×10¹², 8×10¹², 9×10¹², 1×10¹³, 2×10¹³, 3×10¹³, 4×10¹³, 5×10¹³, 6×10¹³, 7× 1013, 8×10¹³, 9×10¹³, 1×10¹⁴, 2×10^{14, 3×10}¹⁴, 4×10^{14, 5×10}¹⁴, 6×10^{14, 7×10}¹⁴, 8×10^{14, 9}× 1014, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG.

### Devices

As used herein, the term “device” refers to any article constructed or modified to suit a particular purpose, such as facilitating the delivery of the pharmaceutical compositions to a subject or the detection of the administered pharmaceutical compositions in a subject.

In some embodiments, the devices may be utilized for intraparenchymal injection of the pharmaceutical compositions. Devices may also be used to administer the pharmaceutical compositions to the spinal cord.

In some embodiments, the device may be a custom floating cannula. In certain embodiments, the custom infusion cannula with a narrow diameter is used for the injections. The cannula may include a 30-gauge beveled needle of fixed length connected to a 30-gauge flexible silastic tubing of variable length. The distal end may be fitted with a Hamilton luer lock, which, in turn, may be attached to a microinjector pump. The proximal silastic tubing may be ensheathed within a 24-gauge rigid outer cannula that is seated on the proximal end of the injection needle flange. The flange seats the outer cannula and may serve as a depth stop for the injection needle

In certain embodiments, the device may be an intraspinal cannula. The intraspinal cannula may include proximal syringe connection and a distal tip. The proximal syringe connection comprises a female luer lock syringe connector which may be connected to a 3-20′ cannula with protective sheathing. The cannula may include a single internal lumen from the distal tip to the syringe. The cannula may include a 4-6″ flexible portion near the distal tip. The distal tip includes a flange/depth stop and a blunt rigid tip. The intraspinal cannula may also include a mechanism for attachment to the subject.

In certain embodiments, the device may be a complex stereotactic frame.

In certain embodiments, the device may be a simplified stereotactic frame.

In certain embodiments, the pharmaceutical compositions may be delivered without a frame.

In certain embodiments, the device may be magnetic resonance imager. Such imagers when used in conjunction with contrast agents such as Gadolinium can detect the administered pharmaceutical compositions in a subject.

In certain embodiments, any of the devices described herein may be combined to deliver and/detect the administered pharmaceutical compositions.

### Combinations

The AAV particles may be used in combination with one or more other therapeutic, prophylactic, diagnostic, or imaging agents. The phrase “in combination with,” is not intended to require that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of pharmaceutical, prophylactic, diagnostic, or imaging compositions in combination with agents that may improve their bioavailability, reduce and/or modify their metabolism, and/or modify their distribution within the body.

The therapeutic agents may be approved by the US Food and Drug Administration or may be in clinical trial or at the preclinical research stage. The therapeutic agents may utilize any therapeutic modality known in the art, with non-limiting examples including gene silencing or interference (i.e., miRNA, siRNA, RNAi, shRNA), gene editing (i.e., TALEN, CRISPR/Cas9 systems, zinc finger nucleases), and gene, protein or enzyme replacement.

### Measurement of Expression

In some embodiments, the administration further comprises evaluating, e.g., measuring, the level of SOD1 expression, e.g., SOD1 gene, SOD1 mRNA, and/or SOD1 protein expression, in the subject, e.g., in a cell, tissue, or fluid, of the subject. The measuring of the level of SOD1 expression may be performed prior to, during, or subsequent to treatment with the AAV particle.

Expression of the SOD1 targeting siRNA from viral genomes may be determined using various methods known in the art such as, but not limited to immunochemistry (e.g., IHC), enzyme-linked immunosorbent assay (ELISA), affinity ELISA, ELISPOT, flow cytometry, immunocytology, surface plasmon resonance analysis, kinetic exclusion assay, liquid chromatography-mass spectrometry (LCMS), high-performance liquid chromatography (HPLC), BCA assay, immunoelectrophoresis, Western blot, SDS-PAGE, protein immunoprecipitation, PCR, and/or in situ hybridization (ISH). In some embodiments, nucleic acids encoding the SOD1 targeting siRNA delivered in different AAV capsids may have different expression levels in different CNS tissues.

In certain embodiments, the SOD1 targeting siRNA is detectable by Western blot.

### IX. Kits and Devices

### Kits

In some aspects, the present disclosure provides a variety of kits for conveniently and/or effectively carrying out methods of the present disclosure. Typically, kits will comprise sufficient amounts and/or numbers of components to allow a user to perform multiple treatments of a subject(s) and/or to perform multiple experiments.

Any of the vectors, constructs, or SOD1 targeting siRNAs of the present disclosure may be comprised in a kit. In some embodiments, kits may further include reagents and/or instructions for creating and/or synthesizing compounds and/or compositions of the present disclosure. In some embodiments, kits may also include one or more buffers. In some embodiments, kits of the disclosure may include components for making protein or nucleic acid arrays or libraries and thus, may include, for example, solid supports.

In some embodiments, kit components may be packaged either in aqueous media or in lyophilized form. The container means of the kits will generally include at least one vial, test tube, flask, bottle, syringe or other container means, into which a component may be placed, and suitably aliquoted. Where there is more than one kit component, (labeling reagent and label may be packaged together), kits may also generally contain second, third or other additional containers into which additional components may be separately placed. In some embodiments, kits may also comprise second container means for containing sterile, pharmaceutically acceptable buffers and/or other diluents. In some embodiments, various combinations of components may be comprised in one or more vial. Kits of the present disclosure may also typically include means for containing compounds and/or compositions of the present disclosure, e.g., proteins, nucleic acids, and any other reagent containers in close confinement for commercial sale. Such containers may include injection or blow-molded plastic containers into which desired vials are retained.

In some embodiments, kit components are provided in one and/or more liquid solutions. In some embodiments, liquid solutions are aqueous solutions, with sterile aqueous solutions being particularly used. In some embodiments, kit components may be provided as dried powder(s). When reagents and/or components are provided as dry powders, such powders may be reconstituted by the addition of suitable volumes of solvent. In some embodiments, it is envisioned that solvents may also be provided in another container means. In some embodiments, labeling dyes are provided as dried powders. In some embodiments, it is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 micrograms or at least or at most those amounts of dried dye are provided in kits of the disclosure. In such embodiments, dye may then be resuspended in any suitable solvent, such as DMSO.

In some embodiments, kits may include instructions for employing kit components as well the use of any other reagent not included in the kit. Instructions may include variations that may be implemented.

### Devices

In some embodiments, compounds and/or compositions of the present disclosure may be combined with, coated onto or embedded in a device. Devices may include, but are not limited to, dental implants, stents, bone replacements, artificial joints, valves, pacemakers and/or other implantable therapeutic device.

The present disclosure provides for devices which may incorporate viral vectors that encode one or more SOD1 targeting polynucleotide. These devices contain in a stable formulation the viral vectors which may be immediately delivered to a subject in need thereof, such as a human patient.

Devices for administration may be employed to deliver the viral vectors encoding the SOD1 targeting polynucleotide of the present disclosure according to single, multi- or split-dosing regimens taught herein.

Method and devices known in the art for multi-administration to cells, organs and tissues arc contemplated for use in conjunction with the methods and compositions disclosed herein as embodiments of the present disclosure.

### X. Definitions

At various places in the present specification, substituents of compounds of the present disclosure are disclosed in groups or in ranges. It is specifically intended that the present disclosure include each and every individual sub-combination of the members of such groups and ranges. The following is a non-limiting list of term definitions.

Articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

Adeno-associated virus: As used herein, the term “adeno-associated virus” or “AAV” refers to members of the dependovirus genus or a variant, e.g., a functional variant, thereof. In some embodiments, the AAV is wildtype, or naturally occurring. In some embodiments, the AAV is recombinant.

AAV Particle: As used herein, an “AAV particle” refers to a particle or a virion comprising an AAV capsid, e.g., an AAV capsid variant, and a polynucleotide, e.g., a viral genome or a vector genome. In some embodiments, the viral genome of the AAV particle comprises at least one payload region and at least one ITR. In some embodiments, an AAV particle of the disclosure is an AAV particle comprising an AAV capsid polypeptide, e.g., a parent capsid sequence with at least one peptide insert. In some embodiments, the AAV particle is capable of delivering a nucleic acid, e.g., a payload region, encoding a payload to cells, typically, mammalian, e.g., human, cells. In some embodiments, an AAV particle of the present disclosure may be produced recombinantly. In some embodiments, an AAV particle may be derived from any serotype, described herein or known in the art, including combinations of serotypes (e.g., “pseudotyped” AAV) or from various genomes (e.g., single stranded or self-complementary). In some embodiments, the AAV particle may be replication defective and/or targeted. In some embodiments, the AAV particle may comprises a peptide present, e.g., inserted into, the capsid to enhance tropism for a desired target tissue. It is to be understood that reference to the AAV particle of the disclosure also includes pharmaceutical compositions thereof, even if not explicitly recited.

Amelioration: As used herein, the term “amelioration” or “ameliorating” refers to a lessening of severity of at least one indicator of a condition or disease. For example, in the context of a neurodegenerative disorder, amelioration includes the reduction or stabilization of neuron loss.

Antisense strand: As used herein, the term “antisense strand” or “the first strand” or “the guide strand” of a siRNA molecule refers to a strand that is substantially complementary to a target sequence. In some embodiments, the antisense strand is substantially complementary to a section of about 10-50 nucleotides, e.g., about 15-30, 16-25, 18-23 or 19-22 nucleotides of the mRNA of the gene targeted for silencing. In some embodiments, the antisense strand is sufficiently complementary to the desired target mRNA sequence to direct target-specific silencing, e.g., complementarity sufficient to trigger the destruction of the desired target mRNA by the RNAi machinery or process.

Approximately: As used herein, the term “approximately” or “about,” as applied to one or more values of interest, refers to a value that is similar to a stated reference value. When referring to a measurable value such as an amount, a temporal duration, and the like, the term is meant to encompass is meant to encompass variations of +20% or in some instances±10%, or in some instances±5%, or in some instances±1%, or in some instances±0.1% from the specified value, as such variations are appropriate to perform the disclosed methods.

Capsid: As used herein, the term “capsid” refers to the exterior, e.g., a protein shell, of a virus particle, e.g., an AAV particle, that is substantially (e.g., >50%, >90%, or 100%) protein. In some embodiments, the capsid is an AAV capsid comprising an AAV capsid protein described herein, e.g., a VP1, VP2, and/or VP3 polypeptide. The AAV capsid protein can be a wild-type AAV capsid protein or a variant, e.g., a structural and/or functional variant from a wild-type or a reference capsid protein, referred to herein as an “AAV capsid variant.” In some embodiments, the AAV capsid variant described herein has the ability to enclose, e.g., encapsulate, a viral genome and/or is capable of entry into a cell, e.g., a mammalian cell. In some embodiments, the AAV capsid variant described herein may have modified tropism compared to that of a wild-type AAV capsid, e.g., the corresponding wild-type capsid.

Encapsulate: As used herein, the term “encapsulate” means to enclose, surround or encase. As an example, a capsid protein, e.g., an AAV capsid variant, often encapsulates a viral genome. In some embodiments, encapsulate within a capsid, e.g., an AAV capsid variant, encompasses 100% coverage by a capsid, as well as less than 100% coverage, e.g., 95% or less. For example, gaps or discontinuities may be present in the capsid so long as the viral genome is retained in the capsid, e.g., prior to entry into a cell.

Complementary and substantially complementary: As used herein, the term “complementary” refers to the ability of polynucleotides to form base pairs with one another. Base pairs are typically formed by hydrogen bonds between nucleotide units in antiparallel polynucleotide strands. Complementary polynucleotide strands can form base pairs in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenine. However, when a U is denoted in the context of the present disclosure, the ability to substitute a T is implied, unless otherwise stated. Perfect complementarity or 100% complementarity refers to the situation in which each nucleotide unit of one polynucleotide strand can form a hydrogen bond with a nucleotide unit of a second polynucleotide strand. Less than perfect complementarity refers to the situation in which some, but not all, nucleotide units of two strands can form hydrogen bond with each other. For example, for two 20-mers, if only two base pairs on each strand can form a hydrogen bond with each other, the polynucleotide strands exhibit 10% complementarity. In the same example, if 18 base pairs on each strand can form hydrogen bonds with each other, the polynucleotide strands exhibit 90% complementarity. The term “complementary” as used herein can encompass fully complementary, partially complementary, or substantially complementary. As used herein, the term “substantially complementary” means that the siRNA has a sequence (e.g., in the antisense strand) which is sufficient to bind the desired target mRNA, and to trigger the RNA silencing of the target mRNA. “Fully complementary”, “perfect complementarity”, or “100% complementarity” refers to the situation in which each nucleotide unit of one polynucleotide or oligonucleotide strand can base-pair with a nucleotide unit of a second polynucleotide or oligonucleotide strand.

Conservative amino acid substitution: As used herein, a “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Conserved: As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In certain embodiments, two or more sequences are said to be “completely conserved” if they are 100% identical to one another. In certain embodiments, two or more sequences are said to be “highly conserved” if they are at least 70% identical, at least 80% identical, at least 90% identical, or at least 95% identical to one another. In certain embodiments, two or more sequences are said to be “highly conserved” if they are about 70% identical, about 80% identical, about 90% identical, about 95%, about 98%, or about 99% identical to one another. In certain embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In certain embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of an polynucleotide or polypeptide or may apply to a portion, region or feature thereof.

Effective amount: As used herein, the term “effective amount” of an agent is that amount sufficient to effect beneficial or desired results, for example, upon single or multiple dose administration to a subject or a cell, in curing, alleviating, relieving or improving one or more symptoms of a disorder and, as such, an “effective amount” depends upon the context in which it is being applied. For example, in the context of administering an agent that treats amyotrophic lateral sclerosis (ALS) and related disorders, an effective amount of an agent is, for example, an amount sufficient to achieve treatment, as defined herein, of a SOD1-related disorder as compared to the response obtained without administration of the agent.

Expression: As used herein, “expression” of a nucleic acid sequence refers to production of an RNA template from a DNA sequence (e.g., by transcription). In some embodiments, expression further comprises one or more of: (1) processing of an RNA transcript (e.g., by splicing, editing, 5′ cap formation, and/or 3′ end processing); (2) translation of an RNA into a polypeptide or protein; and (3) post-translational modification of a polypeptide or protein . . .

Fragment: A “fragment,” as used herein, refers to a portion. For example, an antibody fragment may comprise a CDR, or a heavy chain variable region, or a scFv, etc. In some embodiments, a fragment is a nucleic acid fragment . . .

Homology: As used herein, the term “homology” refers to the overall relatedness between polymeric molecules, e.g. between polynucleotide molecules (e.g. DNA molecules and/or RNA molecules) and/or between polypeptide molecules. In certain embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the present disclosure, two polynucleotide sequences are considered to be homologous if the polypeptides they encode are at least about 50%, 60%, 70%, 80%, 90%, 95%, or even 99% for at least one stretch of at least about 20 amino acids. In certain embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the present disclosure, two protein sequences are considered to be homologous if the proteins are at least about 50%, 60%, 70%, 80%, or 90% identical for at least one stretch of at least about 20 amino acids.

Identity: As used herein, “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are identical at that position. The identity between two sequences is a direct function of the number of matching positions; e.g., if half (e.g., five positions in a polymer ten subunits in length) of the positions in two sequences are identical, the two sequences are 50% identical; if 90% of the positions (e.g., 9 of 10), are matched, the two sequences are 90% identical. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; the contents of each of which are incorporated herein by reference in their entirety. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12 (1), 387 (1984)), BLASTP, BLASTN, and FASTA Altschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product can be an RNA transcribed from the gene (e.g., an mRNA) or a polypeptide translated from an mRNA transcribed from the gene. Typically, a reduction in the level of an mRNA results in a reduction in the level of a polypeptide translated therefrom. The level of expression may be determined using standard techniques for measuring mRNA or protein.

Isolated: As used herein, the term “isolated” refers to a substance or entity that is altered or removed from the natural state, e.g., altered or removed from at least some of component with which it is associated in the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. Such polynucleotides could be part of a vector and/or such polynucleotides or polypeptides could be part of a composition, and still be isolated in that such vector or composition is not part of the environment in which it is found in nature. In some embodiments, an isolated nucleic acid is recombinant, e.g., incorporated into a vector.

miR binding site series: As used herein, a “miR binding site” comprises a nucleic acid sequence (whether RNA or DNA, e.g., differ by “U” of RNA or “T” in DNA) that is capable of binding, or binds, in whole or in part to a microRNA (miR), e.g., through complete or partial hybridization. Typically, such binding occurs between the miR and the miR binding site in the reverse complement orientation. In some embodiments, the miR binding site is transcribed from the AAV viral genome encoding the miR binding site.

In some embodiments, a miR binding site may be encoded or transcribed in series. Such a “miR binding site series” or “miR BSs” may include two or more miR binding sites having the same or different nucleic acid sequence.

Modulatory polynucleotide: As used herein, a “modulatory polynucleotide” is any nucleic acid sequence(s) which functions to modulate (either increase or decrease) the level or amount of a target gene expression, transcript expression and/or protein production.

Variant: The term “variant” refers to a polypeptide or polynucleotide that has an amino acid or a nucleotide sequence that is substantially identical, e.g., having at least 70%, 75%, 80%, 85%, 90%, 95%, or 99% sequence identity to a reference sequence. In some embodiments, the variant is a functional variant.

Functional Variant: The term “functional variant” refers to a polypeptide variant or a polynucleotide variant that has at least one activity of the reference sequence.

Insertional Variant: “Insertional variants” when referring to polypeptides are those with one or more amino acids inserted, e.g., immediately adjacent or subsequent, to a position in an amino acid sequence. “Immediately adjacent” or “immediately subsequent” to an amino acid means connected to either the alpha-carboxy or alpha-amino functional group of the amino acid.

Nucleic acid: As used herein, the terms “nucleic acid,” “nucleic acid molecule,” “polynucleotide,” or “polynucleotide molecule” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. In some embodiments, a “nucleic acid,” “nucleic acid molecule,” “polynucleotide,” or “polynucleotide molecule” comprise a nucleotide/nucleoside derivative or analog. Unless otherwise indicated, a particular nucleic acid sequence also implicitly encompasses conservatively modified variants thereof (for example, degenerate codon substitutions, for example, conservative substitutions), alleles, orthologs, SNPs, and complementary sequences as well as the sequence explicitly indicated. Specifically, degenerate codon substitutions, for example, conservative substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

Payload region: As used herein, a “payload region” is any nucleic acid sequence (e.g., within the viral genome) which encodes one or more “payloads” of the disclosure. As non-limiting examples, a payload region may be a nucleic acid sequence within the viral genome of an AAV particle, which encodes a payload, wherein the payload is an RNAi agent or a polypeptide. Payloads of the present disclosure may be, but are not limited to, peptides, polypeptides, proteins, antibodies, RNAi agents, etc.

Pharmaceutically acceptable: The phrase “pharmaceutically acceptable” is employed herein to refer to those compounds, materials, compositions, and/or dosage forms which are suitable for use in contact with the tissues of human beings and animals.

Polypeptide: As used herein, “polypeptide” means a polymer of amino acid residues (natural or unnatural) linked together most often by peptide bonds. The term, as used herein, refers to proteins, polypeptides, and peptides of any size, structure, or function. In some instances, the polypeptide encoded is smaller than about 50 amino acids and the polypeptide is then termed a peptide. If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides and may be associated or linked. The term polypeptide may also apply to amino acid polymers in which one or more amino acid residues are an artificial chemical analogue of a corresponding naturally occurring amino acid.

Polypeptide variant: The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. In some embodiments, a variant comprises a sequence having at least about 50%, at least about 80%, or at least about 90%, identical (homologous) to a native or a reference sequence.

Peptide: As used herein, “peptide” is less than or equal to 50 amino acids long, e.g., about 5, 10, 15, 20, 25, 30, 35, 40, 45, or 50 amino acids long.

Preventing: As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Region: As used herein, the term “region” refers to a zone or general area. In some embodiments, when referring to a protein or protein module, a region may comprise a linear sequence of amino acids along the protein or protein module or may comprise a three-dimensional area, an epitope and/or a cluster of epitopes. In some embodiments, regions comprise terminal regions. As used herein, the term “terminal region” refers to regions located at the ends or termini of a given agent. When referring to proteins, terminal regions may comprise N- and/or C-termini.

In some embodiments, when referring to a polynucleotide, a region may comprise a linear sequence of nucleic acids along the polynucleotide or may comprise a three-dimensional area, secondary structure, or tertiary structure. In some embodiments, regions comprise terminal regions. As used herein, the term “terminal region” refers to regions located at the ends or termini of a given agent. When referring to polynucleotides, terminal regions may comprise 5′ and/or 3′ termini.

RNA or RNA molecule: As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

RNA agent: As used herein, the term “RNAi agent” refers to an RNA molecule, or its derivative, that can induce inhibition, interfering, or “silencing” of the expression of a target gene and/or its protein product. An RNAi agent may knock-out (virtually eliminate or eliminate) expression, or knock-down (lessen or decrease) expression. The RNAi agent may be, but is not limited to, dsRNA, siRNA, shRNA, pre-miRNA, pri-miRNA, miRNA, stRNA, lncRNA, piRNA, or snoRNA.

RNAi: As used herein, the term “RNA interfering” or “RNAi” refers to a sequence specific regulatory mechanism mediated by RNA molecules which results in the inhibition or interfering or “silencing” of the expression of a corresponding protein-coding gene. RNAi has been observed in many types of organisms, including plants, animals and fungi. RNAi occurs in cells naturally to remove foreign RNAs (e.g., viral RNAs). Natural RNAi proceeds via fragments cleaved from free dsRNA which direct the degradative mechanism to other similar RNA sequences. RNAi is controlled by the RNA-induced silencing complex (RISC) and is initiated by short/small dsRNA molecules in cell cytoplasm, where they interact with the catalytic RISC component argonaute. The dsRNA molecules can be introduced into cells exogenously. Exogenous dsRNA initiates RNAi by activating the ribonuclease protein Dicer, which binds and cleaves dsRNAs to produce double-stranded fragments of 21-25 base pairs with a few unpaired overhang bases on each end. These short double stranded fragments are called small interfering RNAs (siRNAs).

Sample: As used herein, the term “sample” or “biological sample” refers to a subset of its tissues, cells, nucleic acids, or component parts (e.g., body fluids, including but not limited to blood, serum, mucus, lymphatic fluid, synovial fluid, cerebrospinal fluid, saliva, amniotic fluid, amniotic cord blood, urine, vaginal fluid, and semen).

Sense strand: As used herein, the term “the sense strand” or “the second strand” or “the passenger strand” of a siRNA molecule refers to a strand that is complementary to the antisense strand or first strand. The antisense and sense strands of a siRNA molecule are hybridized to form a duplex structure. As used herein, a “siRNA duplex” includes a siRNA strand having sufficient complementarity to a section of about 10-50 nucleotides of the mRNA of the gene targeted for silencing and a siRNA strand having sufficient complementarity to form a duplex with the siRNA strand. According to the present disclosure, recombinant AAV genome may encode a sense and/or antisense strand.

siRNA: As used herein, the terms “short interfering RNA,” “small interfering RNA” or “siRNA” refer to an RNA molecule (or RNA analog) comprising between about 5-60 nucleotides (or nucleotide analogs) which is capable of directing or mediating RNAi. Preferably, a siRNA molecule comprises between about 15-30 nucleotides or nucleotide analogs, such as between about 16-25 nucleotides (or nucleotide analogs), between about 18-23 nucleotides (or nucleotide analogs), between about 19-22 nucleotides (or nucleotide analogs) (e.g., 19, 20, 21 or 22 nucleotides or nucleotide analogs), between about 19-25 nucleotides (or nucleotide analogs), and between about 19-24 nucleotides (or nucleotide analogs). The term “short” siRNA refers to a siRNA comprising 5-23 nucleotides, preferably 21 nucleotides (or nucleotide analogs), for example, 19, 20, 21 or 22 nucleotides. The term “long” siRNA refers to a siRNA comprising 24-60 nucleotides, preferably about 24-25 nucleotides, for example, 23, 24, 25 or 26 nucleotides. Short siRNAs may, in some instances, include fewer than 19 nucleotides, e.g., 16, 17 or 18 nucleotides, or as few as 5 nucleotides, provided that the shorter siRNA retains the ability to mediate RNAi. Likewise, long siRNAs may, in some instances, include more than 26 nucleotides, e.g., 27, 28, 29, 30, 35, 40, 45, 50, 55, or even 60 nucleotides, provided that the longer siRNA retains the ability to mediate RNAi or translational repression absent further processing, e.g., enzymatic processing, to a short siRNA. siRNAs can be single stranded RNA molecules (ss-siRNAs) or double stranded RNA molecules (ds-siRNAs) comprising a sense strand and an antisense strand which hybridized to form a duplex structure called an siRNA duplex.

Spacer: As used here, a “spacer” is generally any selected nucleic acid sequence of, e.g., 1, 2, 3, 4, 5, 6, 7, 8, 9, or 10 nucleotides in length, which is located between two or more consecutive miR binding site sequences. Spacers may also be more than 10 nucleotides in length, e.g., 20, 30, 40, or 50 or more than 50 nucleotides.

Subject: As used herein, the term “subject” or “patient” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants.

Targeted Cells: As used herein, “target cells” or “target tissue” refers to any one or more cells of interest. The cells may be found in vitro, in vivo, in situ or in the tissue or organ of an organism. In some embodiments, the organism may be an animal, e.g. a mammal, e.g., a human, e.g., a human patient.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In some embodiments, a therapeutically effective amount is provided in a single dose.

Treating: As used herein, the term “treating” refers to partially or completely alleviating, ameliorating, improving, relieving, reversing, delaying onset of, inhibiting progression of, reducing severity of, and/or reducing incidence of one or more symptoms or features of a particular infection, disease, disorder, and/or condition. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Vector: As used herein, the term “vector” refers to any molecule or moiety which transports, transduces, or otherwise acts as a carrier of a heterologous molecule. In some embodiments, vectors may be plasmids. Vectors of the present disclosure may be produced recombinantly. The heterologous molecule may be a polynucleotide and/or a polypeptide.

Viral Genome: As used herein, the term “viral genome” refers to the nucleic acid sequence(s) encapsulated in an AAV particle. A viral genome comprises a nucleic acid sequence with at least one payload region encoding a payload and at least one ITR.

The present disclosure is further illustrated by the following non-limiting examples.

### Examples

### Example 1. SOD1 Targeting Polynucleotide Design (siRNA)

siRNA design is carried out to identify siRNAs targeting human SOD1 gene. The design uses the SOD1 transcripts from human (GenBank access No. NM_000454.4 (SEQ ID NO: 110)), cynomolgus (GenBank access No. NM_001285406.1 (SEQ ID NO: 111)), rhesus SOD1 transcript (GenBank access No. NM_001032804.1 (SEQ ID NO: 4822)), and Sus scrofa (GenBank access No. NM_001190422.1 (SEQ ID NO: 112)), respectively (Table 19). The siRNA duplexes are designed with 100% identity to the human SOD1 transcript for positions 2-18 of the antisense strand, and partial or 100% identity to the non-human SOD1 transcript for positions 2-18 of the antisense strand.

In all siRNA duplexes, position 1 of the antisense strand is engineered to a U and position 19 of the sense strand is engineered to a C, in order to unpair the duplex at this position.

TABLE 19
SOD1 gene sequences

| SOD1 | Access | SEQ ID | |
|---|---|---|---|
| transcripts | No. | NO. | Sequence |
| Human (Homo | NM_000454.4 | 110 | GTTTGGGGCCAGAGTGGGCGAGGCGCGGAGGTCTGGCCTATAAAGT |
| sapiens) SOD1 | | | AGTCGCGGAGACGGGGTGCTGGTTTGCGTCGTAGTCTCCTGCAGCG |
| cDNA (981 bp) | | | TCTGGGGTTTCCGTTGCAGTCCTCGGAACCAGGACCTCGGCGTGGC |
| | | | CTAGCGAGTTATGGCGACGAAGGCCGTGTGCGTGCTGAAGGGCGAC |
| | | | GGCCCAGTGCAGGGCATCATCAATTTCGAGCAGAAGGAAAGTAATG |
| | | | GACCAGTGAAGGTGTGGGGAAGCATTAAAGGACTGACTGAAGGCCT |
| | | | GCATGGATTCCATGTTCATGAGTTTGGAGATAATACAGCAGGCTGT |
| | | | ACCAGTGCAGGTCCTCACTTTAATCCTCTATCCAGAAAACACGGTG |
| | | | GGCCAAAGGATGAAGAGAGGCATGTTGGAGACTTGGGCAATGTGAC |
| | | | TGCTGACAAAGATGGTGTGGCCGATGTGTCTATTGAAGATTCTGTG |
| | | | ATCTCACTCTCAGGAGACCATTGCATCATTGGCCGCACACTGGTGG |
| | | | TCCATGAAAAAGCAGATGACTTGGGCAAAGGTGGAAATGAAGAAAG |
| | | | TACAAAGACAGGAAACGCTGGAAGTCGTTTGGCTTGTGGTGTAATT |
| | | | GGGATCGCCCAATAAACATTCCCTTGGATGTAGTCTGAGGCCCCTT |
| | | | AACTCATCTGTTATCCTGCTAGCTGTAGAAATGTATCCTGATAAAC |
| | | | ATTAAACACTGTAATCTTAAAAGTGTAATTGTGTGACTTTTTCAGA |
| | | | GTTGCTTTAAAGTACCTGTAGTGAGAAACTGATTTATGATCACTTG |
| | | | GAAGATTTGTATAGTTTTATAAAACTCAGTTAAAATGTCTGTTTCA |
| | | | ATGACCTGTATTTTGCCAGACTTAAATCACAGATGGGTATTAAACT |
| | | | TGTCAGAATTTCTTTGTCATTCAAGCCTGTGAATAAAAACCCTGTA |
| | | | TGGCACTTATTATGAGGCTATTAAAAGAATCCAAATTCAAACTAAA |
| | | | AAAAAAAAAAAAAAA |
| Cynomolgus | NM_001285406.1 | 111 | ATGGCGATGAAGGCCGTGTGCGTGTTGAAGGGCGACAGCCCAGTGC |
| (Macaca | | | AGGGCACCATCAATTTCGAGCAGAAGGAAAGTAATGGACCAGTGAA |
| fascicularis) | | | GGTGTGGGGAAGCATTACAGGATTGACTGAAGGCCTGCATGGATTC |
| SOD1 cDNA | | | CATGTTCATCAGTTTGGAGATAATACACAAGGCTGTACCAGTGCAG |
| (465 bp) | | | GTCCTCACTTTAATCCTCTATCCAGACAACACGGTGGGCCAAAGGA |
| | | | TGAAGAGAGGCATGTTGGAGACCTGGGCAATGTGACTGCTGGCAAA |
| | | | GATGGTGTGGCCAAGGTGTCTTTCGAAGATTCTGTGATCTCGCTCT |
| | | | CAGGAGACCATTCCATCATTGGCCGCACATTGGTGGTCCATGAAAA |
| | | | AGCAGATGACTTGGGCAAAGGTGGAAATGAAGAAAGTAAAAAGACA |
| | | | GGAAACGCTGGAGGTCGTCTGGCTTGTGGTGTAATTGGGATCGCCC |
| | | | AATAA |
| rhesus (Macaca | NM_001032804.1 | 4822 | ATGGCGATGAAGGCCGTGTGCGTGTTGAAGGGCGACAGCCCAGTGC |
| mulatta) SOD1 | | | AGGGCACCATCAATTTCGAGCAGAAGGAAAGTAATGGACCAGTGAA |
| cDNA (465 bp) | | | GGTGTGGGGAAGCATTACAGGATTGACTGAAGGCCTGCATGGATTC |
| | | | CATGTTCATCAGTTTGGAGATAATACACAAGGCTGTACCAGTGCAG |
| | | | GTCCTCACTTTAATCCTCTATCCAGACAACACGGTGGGCCAAAGGA |
| | | | TGAAGAGAGGCATGTTGGAGACCTGGGCAATGTGACTGCTGGCAAA |
| | | | GATGGTGTGGCCAAGGTGTCTTTCGAAGATTCTGTGATCTCGCTCT |
| | | | CAGGAGACCATTCCATCATTGGCCGCACATTGGTGGTCCATGAAAA |
| | | | AGCAGATGACTTGGGCAAAGGTGGAAATGAAGAAAGTAAAAAGACA |
| | | | GGAAACGCTGGAGGTCGTCTGGCTTGTGGTGTAATTGGGATCGCCC |
| | | | AATAA |
| Pig (Sus scrofa) | NM_001190422.1 | 112 | CGTCGGCGTGTACTGCGGCCTCTCCCGCTGCTTCTGGTACCCTCCC |
| SOD1 cDNA | | | AGCCCGGACCGGAGCGCGCCCCCGCGAGTCATGGCGACGAAGGCCG |
| (658 bp) | | | TGTGTGTGCTGAAGGGCGACGGCCCGGTGCAGGGCACCATCTACTT |
| | | | CGAGCTGAAGGGAGAGAAGACAGTGTTAGTAACGGGAACCATTAAA |
| | | | GGACTGGCTGAAGGTGATCATGGATTCCATGTCCATCAGTTTGGAG |
| | | | ATAATACACAAGGCTGTACCAGTGCAGGTCCTCACTTCAATCCTGA |
| | | | ATCCAAAAAACATGGTGGGCCAAAGGATCAAGAGAGGCACGTTGGA |
| | | | GACCTGGGCAATGTGACTGCTGGCAAAGATGGTGTGGCCACTGTGT |
| | | | ACATCGAAGATTCTGTGATCGCCCTCTCGGGAGACCATTCCATCAT |
| | | | TGGCCGCACAATGGTGGTCCATGAAAAACCAGATGACTTGGGCAGA |
| | | | GGTGGAAATGAAGAAAGTACAAAGACGGGAAATGCTGGAAGTCGTT |
| | | | TGGCCTGTGGTGTAATTGGGATCACCCAGTAAACATTCCCTCATGC |
| | | | CATGGTCTGAATGCCAGTAACTCATCTGTTATCTTGCTAGTTGTAG |
| | | | TTGTAGAAATTTAACTTGATAAACATTAAACACTGTAACCTTAAAA |
| | | | AAAAAAAAAAAAAA |

### Example 2. High-throughput screen of TRACER AAV library in NHP and Mice

A TRACER based method as described in WO2020072683, WO 2021/202651, and WO2021230987, the contents of which are herein incorporated by reference in their entirety, was used to generate the AAV capsid variants described herein. An orthogonal evolution approach was combined with a high throughput screening by NGS. Briefly, the library of AAV capsid variants was generated using a sliding window approach, where 6 amino acid sequences were inserted into 8 different positions across loop IV of AAV9, including immediately subsequent to positions 453, 454, 455, 456, 457, 458, 459, and 460, relative to a reference sequence numbered according to SEQ ID NO: 138. The initial library was passed twice through non-human primates (NHP, 2-4 years of age). After the second passage (e.g., 28 days post injection into two NHPs), RNA was extracted from six brain regions. Following RNA recovery and RT-PCR amplification, a systematic NGS enrichment analysis was performed to calculate fold enrichment relative to an AAV9 wild-type control. Following these two passages, approximately 21195 variants were identified with an average fold change greater than wild-type. Of the 21195 variants, 1558 demonstrated a fold-change of greater than 6 compared to wild-type and were detected across all brain regions investigated. Of these 1558, approximately 1470 variants were selected for constructing a synthetic library and a third passage through two NHPs. Within the 1470 variants selected for further characterization and investigation, there was a relatively even distribution for each insertion position of the sliding window used to generate the initial library.

After creation of the synthetic library with the sub-selected variants, the synthetic library was screened (passage 3) in two NHPs (2-4 years of age) and two strains of mice, BALB/c (n=3, 6-8 weeks of age) and C57B1/6 mice (n=3, 6-8 weeks of age), in a first cross-species evolution screen. The animals were injected intravenously with the synthetic library. After a period in vivo, (e.g., 28-days) RNA was extracted from nervous tissue, e.g., brain, spinal cord, and DRG of the NHPs and the brains of mice. Following RNA recovery and RT-PCR amplification, a systematic NGS enrichment analysis was performed, and the peptides comprised within the variants were identified and the capsid enrichment ratio for each variant compared to the wild-type AAV9 control was calculated (fold enrichment relative to wild-type AAV9) (Table 21). Values above 1 indicate an increase in expression relative to AAV9. All animals were dosed intravenously at 2-3 VG/kg across the screen.

As shown in Table 21, approximately 700 variants demonstrated an increase in expression relative to AAV9, and several variants demonstrated a greater than 10-fold enrichment relative to AAV9 in the brain of NHPs. Further, the variants demonstrating the greatest fold enrichment in the brain also demonstrated the greatest fold enrichment in the spinal cord relative to AAV9 in NHPs.

These variants also demonstrated de-targeting in the DRG (data not shown). For instance, the variant comprising GSGSPHSKAQNQQT (SEQ ID NO: 200) demonstrated a 76.6 fold enrichment in the brain, a 29.4 fold enrichment in the spinal cord, and 0.4 fold enrichment in the DRG of NHPs relative to AAV9; and GHDSPHKSGQNQQT (SEQ ID NO: 201) demonstrated a 62.6 fold enrichment in the brain, a 15.6 fold enrichment in the spinal cord, and 0.0 fold enrichment in the DRG of NHPs relative to AAV9. Also, across the peptides comprised within the AAV capsid variants with the greatest fold-enrichment in the NHP brain relative wild-type AAV9, it was observed that each of these peptides comprised an SPH motif in the same position (e.g., immediately subsequent to position 455, relative to a reference sequence numbered according to the amino acid sequence of SEQ ID NO: 138), regardless of the insertion position within the variant capsid, as well as a positive amino acid (e.g., K or R) in one of the next three residues subsequent to the SPH motif.

Those variants with the greatest fold enrichment in the brains of NHPs also had the greatest fold enrichment in the brains of both mouse species. Also, when comparing the fold enrichment relative to wild-type for each variant between the two species of mice investigated (C57B1/6 and BALB/c mice), they were highly correlated (R2=0.8591).

TABLE 21
NGS fold-enrichment of AAV capsid variants in NHPs and mice

| | | | Fold | Fold | Fold |
|---|---|---|---|---|---|
| | | Fold | enrichment | enrichment | enrichment |
| | | enrichment | over AAV9 | over AAV9 | over AAV9 |
| | | over AAV9 | in the | in the | in the |
| | SEQ | in the | spinal | brain of | brain of |
| Peptide | ID | brain | cord | Mice | Mice |
| Sequence | NO: | of NHPs | of NHPs | (C57Bl/6) | (BALB/c) |
| GSGSPHSKAQNQQT | 200 | 73.615 | 29.402 | 25.293 | 41.304 |
| GHDSPHKSGQNQQT | 201 | 62.612 | 15.641 | 63.993 | 49.760 |
| GSGSPHARMQNQQT | 202 | 56.138 | 22.690 | 7.795 | 4.164 |
| GSGSPHVKSQNQQT | 203 | 37.551 | 13.649 | 8.069 | 15.861 |
| GQDSPHKSGQNQQT | 204 | 24.569 | 3.548 | 57.344 | 42.615 |
| GSGSPHASRQNQQT | 205 | 18.265 | 7.804 | 28.028 | 36.577 |
| GSGSPHASRQNKQT | 206 | 17.520 | 35.029 | 13.096 | 18.114 |
| GSGSPHVKIQNQQT | 207 | 16.854 | 9.068 | 2.173 | 2.227 |
| GSGSPHSKAKNQQT | 208 | 14.458 | 0.049 | 21.494 | 23.556 |
| GSGSPHKKNQNQQT | 209 | 12.991 | 0.379 | 25.958 | 7.415 |
| GSGSPHVRMQNQQT | 210 | 11.574 | 6.764 | 9.121 | 10.076 |
| GSGSPHASRQKQQT | 211 | 11.417 | 0.005 | 7.413 | 12.400 |
| GHSSPHRSGQNQQT | 212 | 10.357 | 1.887 | 23.197 | 25.442 |
| GMRTYHLSGQNQQT | 213 | 9.241 | 1.939 | 2.033 | 1.586 |
| GSGSPHTRGQNQQT | 214 | 7.092 | 3.815 | 10.801 | 6.240 |
| GSGIIPVSSQNQQT | 215 | 6.352 | 0.000 | 0.642 | 0.253 |
| GSEYGHKSGQNQQT | 216 | 6.308 | 2.750 | 5.198 | 5.332 |
| GRGQNVSSVHRQQT | 217 | 5.404 | 0.000 | 1.206 | 0.691 |
| GSSHRFYGGQNQQT | 218 | 4.732 | 0.000 | 0.787 | 0.110 |
| GYFVAAWSGQNQQT | 219 | 4.488 | 0.000 | 0.071 | 0.175 |
| GSVLHSHAAQNQQT | 220 | 4.150 | 6.448 | 0.675 | 0.423 |
| GSGDLVVSTQNQQT | 221 | 3.874 | 1.177 | 0.411 | 0.273 |
| GSYGMAASGQNQQT | 222 | 3.817 | 10.052 | 1.274 | 0.829 |
| GLNHFGASGQNQQT | 223 | 3.802 | 3.188 | 0.774 | 0.579 |
| GSTGSHSAGQNQHT | 224 | 3.717 | 0.285 | 1.190 | 0.850 |
| GLAGHTVSGQNQQT | 225 | 3.632 | 0.229 | 0.972 | 0.202 |
| GIILGASSGQNQQT | 226 | 3.630 | 4.868 | 1.378 | 0.865 |
| GSGVSTYNIQNQQT | 227 | 3.609 | 2.912 | 0.769 | 0.520 |
| GSLVSVQTGQNQQT | 228 | 3.534 | 6.043 | 0.903 | 0.469 |
| GQSSPHRSGQNQQT | 229 | 3.496 | 2.142 | 12.352 | 19.366 |
| GREYGHKSGQNQQT | 230 | 3.453 | 0.000 | 1.422 | 0.959 |
| GHTLTLSSGQNQQT | 231 | 3.405 | 5.648 | 0.648 | 0.606 |
| GSITLIPSGQNQQT | 232 | 3.361 | 3.917 | 0.326 | 0.435 |
| GSNGFTALGQNQQT | 233 | 3.361 | 2.663 | 0.830 | 0.332 |
| GSGHSSHSVQNQQT | 234 | 3.339 | 3.318 | 0.942 | 0.424 |
| GSGIPQRSGKNQQT | 235 | 3.331 | 0.000 | 1.418 | 1.685 |
| GSGDTLHMLQNQQT | 236 | 3.317 | 1.174 | 0.393 | 0.482 |
| GERHTVLSGQNQQT | 237 | 3.289 | 3.008 | 1.027 | 0.607 |
| GSGMPQSHIQNQQT | 238 | 3.289 | 11.609 | 0.514 | 0.334 |
| GSGQLSGIGGNQQT | 239 | 3.266 | 0.287 | 0.993 | 0.626 |
| GSGQNRKPASFAQT | 240 | 3.204 | 0.000 | 0.892 | 1.061 |
| GSGSVSQLGQNQQT | 241 | 3.184 | 2.307 | 0.596 | 0.375 |
| GSDFLGTHGQNQQT | 242 | 3.171 | 0.348 | 1.038 | 0.750 |
| GQIVQNPSGQNQQT | 243 | 3.133 | 0.406 | 1.446 | 0.635 |
| GSGTQIPSQQNQQT | 244 | 3.112 | 1.224 | 0.470 | 0.151 |
| GSGQNQQSAREGLT | 245 | 3.111 | 5.632 | 1.221 | 1.104 |
| GSGLGMSTGQNQQT | 246 | 3.110 | 5.499 | 0.458 | 0.660 |
| GSGLPVLSGQNQQT | 247 | 3.100 | 4.149 | 0.631 | 0.210 |
| GSGHSIRTDQNQQT | 248 | 3.074 | 15.600 | 0.229 | 0.148 |
| GSGQSVQTVVNQQT | 249 | 3.057 | 5.441 | 0.582 | 0.240 |
| GSGQNRAQSRFQQT | 250 | 3.043 | 0.000 | 0.619 | 1.788 |
| GGGDLGRSSQNQQT | 251 | 3.036 | 4.830 | 0.916 | 0.539 |
| GGGTKMDSGQNQQT | 252 | 3.034 | 0.000 | 0.733 | 0.297 |
| GSGSPHPSRQNQQT | 253 | 3.017 | 1.993 | 1.869 | 0.975 |
| GSGQFTNAGMNQQT | 254 | 2.969 | 0.936 | 0.565 | 0.418 |
| GGRNGHTVGQNQQT | 255 | 2.965 | 3.732 | 1.105 | 1.003 |
| GSGFGPQTGQNQQT | 256 | 2.964 | 2.861 | 1.280 | 0.849 |
| GRTDSHTSGQNQQT | 257 | 2.913 | 1.510 | 1.299 | 0.704 |
| GYEVLGSSGQNQQT | 258 | 2.891 | 0.000 | 2.459 | 0.319 |
| GSVHLSVTGQNQQT | 259 | 2.882 | 1.157 | 0.741 | 0.282 |
| GFMSYKGSGQNQQT | 260 | 2.865 | 0.209 | 1.808 | 0.569 |
| GNIAGSVSGQNQQT | 261 | 2.849 | 1.187 | 0.446 | 0.257 |
| GSGSHRDVSQNQQT | 262 | 2.843 | 4.022 | 0.626 | 0.550 |
| GGLGSMSSGQNQQT | 263 | 2.812 | 1.405 | 1.802 | 0.822 |
| GSGHLPQSAQNQQT | 264 | 2.803 | 7.828 | 0.826 | 0.496 |
| GGVLVGGSGQNQQT | 265 | 2.778 | 0.178 | 1.527 | 0.688 |
| GTHPYTSSGQNQQT | 266 | 2.775 | 1.684 | 0.758 | 0.471 |
| GSGQNQQLKENRST | 267 | 2.765 | 0.062 | 1.149 | 1.118 |
| GSGQNQQTSPHNHT | 268 | 2.761 | 3.132 | 1.524 | 0.845 |
| GSGTLYPQSQNQQT | 269 | 2.761 | 5.558 | 0.324 | 0.160 |
| GSGQNQQSNWITKT | 270 | 2.711 | 0.000 | 0.540 | 0.634 |
| GSGYTSLFLQNQQT | 271 | 2.710 | 0.010 | 0.490 | 1.044 |
| GSGVMTHVLQNQQT | 272 | 2.692 | 0.347 | 0.370 | 0.533 |
| GSVSDVRAGQNQQT | 273 | 2.661 | 1.647 | 0.267 | 0.747 |
| GSGQSHMATLNQQT | 274 | 2.657 | 0.724 | 1.173 | 0.504 |
| GSGLSVHLAQNQQT | 275 | 2.657 | 1.234 | 0.806 | 0.508 |
| GSGLSHATQQNQQT | 276 | 2.640 | 7.819 | 1.111 | 0.638 |
| GSGLSVQSGQNQQT | 277 | 2.637 | 2.929 | 1.695 | 1.005 |
| GSGHMTYREKNQQT | 278 | 2.633 | 5.267 | 1.257 | 0.540 |
| GSKGVPTPGQNQQT | 279 | 2.625 | 1.292 | 1.452 | 0.459 |
| GSGLLPLSSQNQQT | 280 | 2.612 | 1.130 | 0.501 | 0.293 |
| GNGLYAVSGQNQQT | 281 | 2.611 | 9.148 | 0.322 | 0.213 |
| GFNGSPSSGQNQQT | 282 | 2.609 | 12.197 | 2.338 | 0.924 |
| GSGQIRHSDQNQQT | 283 | 2.600 | 12.884 | 1.170 | 0.320 |
| GGQVAPSSGQNQQT | 284 | 2.581 | 2.427 | 1.433 | 0.709 |
| GSFSMHTHGQNQQT | 285 | 2.535 | 0.118 | 1.027 | 0.693 |
| GSGQNQQVIQGSNT | 286 | 2.521 | 8.778 | 0.935 | 0.810 |
| GRVLHSHAGQNQQT | 287 | 2.513 | 0.826 | 1.294 | 0.908 |
| GSGQNQQTSLQDQT | 288 | 2.505 | 0.500 | 0.315 | 0.968 |
| GSGLGRAPVQNQQT | 289 | 2.503 | 2.214 | 0.841 | 0.383 |
| GNGFSSASGQNQQT | 290 | 2.493 | 0.772 | 0.240 | 0.182 |
| GSGQMASRESNQQT | 291 | 2.492 | 0.300 | 0.341 | 0.288 |
| GPGLPNHSGQNQQT | 292 | 2.486 | 1.992 | 1.197 | 0.659 |
| GNIQWQGSGQNQQT | 293 | 2.468 | 6.266 | 1.182 | 0.837 |
| GMSAHMSSGQNQQT | 294 | 2.456 | 5.255 | 1.310 | 0.947 |
| GHSFVNRSGQNQQT | 295 | 2.447 | 11.148 | 1.305 | 0.756 |
| GRAVMDHSGQNQQT | 296 | 2.408 | 3.209 | 0.728 | 0.283 |
| GALTVMQSGQNQQT | 297 | 2.381 | 0.430 | 0.246 | 0.199 |
| GSGQRSPVLPNQQT | 298 | 2.369 | 6.230 | 0.434 | 0.526 |
| GSGQNGHLSLKQQT | 299 | 2.362 | 1.896 | 0.718 | 0.270 |
| GSLPRGTSDQNQQT | 300 | 2.362 | 0.000 | 0.453 | 0.495 |
| GVAGSLVSGQNQQT | 301 | 2.358 | 7.670 | 1.321 | 1.160 |
| GRGGIPQSGQNQQT | 302 | 2.352 | 8.683 | 1.639 | 1.181 |
| GSGQYASSIPNQQT | 303 | 2.346 | 3.321 | 1.022 | 0.489 |
| GTDFGRQSSQNQQT | 304 | 2.346 | 3.196 | 1.021 | 0.797 |
| GIFMQTPSGQNQQT | 305 | 2.344 | 6.198 | 0.938 | 0.252 |
| GSGQNQQTRLVDLT | 306 | 2.342 | 9.348 | 1.268 | 0.490 |
| GTREMPLSGQNQQT | 307 | 2.339 | 2.830 | 1.436 | 0.538 |
| GSRLVHVHGQNQQT | 308 | 2.334 | 1.174 | 1.277 | 0.934 |
| GSGRLVPNGPNQQT | 309 | 2.314 | 3.925 | 0.639 | 0.411 |
| GSGYLRESPQNQQT | 310 | 2.311 | 0.878 | 0.331 | 0.677 |
| GARIQNASGQKQQT | 311 | 2.300 | 2.103 | 1.220 | 1.039 |
| GLSNPMPSGQNQQT | 312 | 2.280 | 6.033 | 1.190 | 0.829 |
| GSTVQDTRGQNQQT | 313 | 2.270 | 4.979 | 0.576 | 0.473 |
| GPFGMPSSGQNQQT | 314 | 2.260 | 2.700 | 0.727 | 0.560 |
| GSGQNHGVLSNQQT | 315 | 2.254 | 1.603 | 1.113 | 0.701 |
| GSGYSMSQAQNQQT | 316 | 2.250 | 4.479 | 0.519 | 0.329 |
| GSGMLTHTLQNQQT | 317 | 2.246 | 2.272 | 0.496 | 0.199 |
| GRGSPHASRQNQQT | 318 | 2.241 | 0.000 | 5.050 | 5.856 |
| GLSWPSTSGQNQQT | 319 | 2.238 | 0.000 | 0.910 | 0.610 |
| GNSMERTSGQNQQT | 320 | 2.221 | 4.177 | 1.047 | 0.935 |
| GSGMSPSTLQNQQT | 321 | 2.216 | 3.053 | 0.318 | 0.153 |
| GSGHGQVLSQNQQT | 322 | 2.213 | 12.133 | 1.880 | 0.661 |
| GRGQIYSTGGNQQT | 323 | 2.210 | 11.629 | 1.329 | 0.743 |
| GVVAAHNSGQNQQT | 324 | 2.202 | 1.301 | 1.196 | 1.336 |
| GDSSLRHSGQNQQT | 325 | 2.194 | 0.000 | 0.662 | 0.412 |
| GSLVSQGAGQNQQT | 326 | 2.188 | 4.414 | 1.436 | 1.246 |
| GSLLQAHSGQNQQT | 327 | 2.182 | 1.008 | 0.575 | 0.748 |
| GSGHIYVGIQNQQT | 328 | 2.178 | 6.428 | 0.989 | 0.337 |
| GHHTTVQSGQNQQT | 329 | 2.177 | 6.245 | 0.851 | 0.755 |
| GSRQSKRNELNQQT | 330 | 2.177 | 0.000 | 1.325 | 0.232 |
| GSGQNQQHVSSPRT | 331 | 2.176 | 1.279 | 1.847 | 0.938 |
| GSSKELLWGQNQQT | 332 | 2.163 | 0.000 | 0.506 | 0.883 |
| GSLSTPSSGQNQQT | 333 | 2.159 | 1.279 | 1.094 | 0.669 |
| GSIGYAGQGQNQQT | 334 | 2.157 | 4.951 | 1.604 | 0.712 |
| GSGQNQRVSNSQQT | 335 | 2.146 | 0.492 | 1.086 | 0.985 |
| GSGYASHVQQNQQT | 336 | 2.146 | 3.038 | 1.157 | 0.758 |
| GSGEYSRSGQNQQT | 337 | 2.145 | 0.745 | 0.617 | 0.205 |
| GSVSTHSSGQNQQT | 338 | 2.145 | 3.446 | 1.198 | 0.918 |
| GSGQNQHSLGNYQT | 339 | 2.143 | 1.896 | 1.077 | 0.606 |
| GSGGLDTRGQNQQT | 340 | 2.139 | 6.216 | 0.236 | 0.197 |
| GNILHATSGQNQQT | 341 | 2.136 | 0.125 | 1.159 | 0.424 |
| GSGQSYTMTQNQQT | 342 | 2.136 | 6.755 | 0.297 | 0.231 |
| GSGQNQHSAPNSQT | 343 | 2.134 | 4.143 | 1.187 | 0.731 |
| GSGQNQQTMDHNRT | 344 | 2.130 | 4.944 | 0.642 | 0.440 |
| GSNGGVGTGQNQQT | 345 | 2.130 | 0.788 | 1.191 | 1.087 |
| GAGSIIPSGQNQQT | 346 | 2.129 | 7.164 | 0.595 | 0.249 |
| GSGQTHGGQHNQQT | 347 | 2.125 | 12.251 | 1.448 | 1.098 |
| GSNLSFQSGQNQQT | 348 | 2.122 | 5.853 | 1.087 | 0.719 |
| GATLQVHSGQNQQT | 349 | 2.122 | 2.219 | 0.623 | 0.545 |
| GSGFNQRSEQNQQT | 350 | 2.121 | 4.491 | 1.770 | 0.758 |
| GSGSLRDFDQNQQT | 351 | 2.120 | 6.846 | 0.586 | 0.272 |
| GSGDSITGKQNQQT | 352 | 2.112 | 1.295 | 0.793 | 0.306 |
| GSGQDRNIVQNQQT | 353 | 2.112 | 0.229 | 0.454 | 0.632 |
| GSGLSHSHQQNQQT | 354 | 2.109 | 5.852 | 1.256 | 0.592 |
| GSGQNQQTGMSSVK | 355 | 2.109 | 4.544 | 1.451 | 0.679 |
| GSVTHGISGQNQQT | 356 | 2.105 | 4.542 | 1.135 | 0.789 |
| GVVAHQPSGQNQQT | 357 | 2.103 | 0.152 | 0.910 | 2.267 |
| GSGPILGQLQNQQT | 358 | 2.097 | 2.058 | 0.470 | 0.123 |
| GSGHVPNSGLNQQT | 359 | 2.091 | 0.653 | 1.636 | 1.154 |
| GDAGVRSSGQNQQT | 360 | 2.068 | 3.918 | 1.033 | 1.193 |
| GSGSQLMSLQNQQT | 361 | 2.065 | 3.559 | 0.563 | 0.172 |
| GSGLDYSQRQNQQT | 362 | 2.056 | 0.837 | 0.484 | 0.217 |
| GSGQSSGRLINKQT | 363 | 2.055 | 28.135 | 0.543 | 0.277 |
| GSSVSPSSGQNQQT | 364 | 2.054 | 0.579 | 1.064 | 0.787 |
| GSGQVVGLSGNQQT | 365 | 2.052 | 7.212 | 0.785 | 0.881 |
| GSNMGVPLGQNQQT | 366 | 2.049 | 2.448 | 0.334 | 0.420 |
| GSFYPSSTGQNQQT | 367 | 2.047 | 2.374 | 0.420 | 0.277 |
| GSGQNQQTRLTDLT | 368 | 2.046 | 8.470 | 0.910 | 0.776 |
| GPTNGRSSGQNQQT | 369 | 2.034 | 8.903 | 0.936 | 1.308 |
| GSGLLHGKLQNQQT | 370 | 2.032 | 2.521 | 1.068 | 0.917 |
| GANMGHVSGQNQQT | 371 | 2.020 | 0.810 | 1.302 | 1.138 |
| GSGQNQQSGRGDLT | 372 | 2.019 | 6.919 | 0.524 | 1.152 |
| GSHGHYASGQKQQT | 373 | 2.016 | 0.000 | 0.895 | 0.685 |
| GSGDLRISPQNQQT | 374 | 2.012 | 16.207 | 0.620 | 0.237 |
| GSGMPVILGQNQQT | 375 | 2.005 | 0.150 | 0.840 | 0.287 |
| GRGVITSSGQNHQT | 376 | 2.004 | 0.864 | 1.656 | 0.669 |
| GSGHSVSGPQNQQT | 377 | 1.993 | 6.259 | 1.370 | 0.619 |
| GSRNGHTVGRNQQT | 378 | 1.993 | 0.000 | 1.162 | 0.367 |
| GAGVHMVSGQNQQT | 379 | 1.987 | 6.488 | 1.055 | 0.791 |
| GSGQNHRPSVLQQT | 380 | 1.983 | 5.582 | 0.433 | 0.582 |
| GSGSPRDSIQNQQT | 381 | 1.981 | 4.914 | 0.171 | 0.446 |
| GSGQGIHSSVNQQT | 382 | 1.981 | 4.873 | 0.632 | 0.634 |
| GSGQQLSITPNQQT | 383 | 1.979 | 10.280 | 0.845 | 0.201 |
| GGYHSQTSGQNQQT | 384 | 1.978 | 2.642 | 1.740 | 1.525 |
| GSLHHDNHGQNQQT | 385 | 1.976 | 0.980 | 0.968 | 0.463 |
| GIMARDSSGQNQQT | 386 | 1.972 | 3.486 | 1.320 | 0.904 |
| GVVHITNSGQNQQT | 387 | 1.969 | 0.504 | 0.794 | 0.846 |
| GSGQNQHSAPFNQT | 388 | 1.969 | 0.499 | 0.759 | 0.870 |
| GSGQTSGLKQNQQT | 389 | 1.968 | 3.927 | 0.394 | 0.334 |
| GSGQNQQTSLSNTA | 390 | 1.959 | 1.186 | 1.567 | 1.182 |
| GSGQNQAVHNKSQT | 391 | 1.956 | 3.791 | 1.465 | 1.083 |
| GVHTHLPSGQNQQT | 392 | 1.952 | 1.364 | 1.414 | 0.796 |
| GHLTMHNSGQNHQT | 393 | 1.938 | 1.798 | 1.030 | 0.586 |
| GSGSSSRPYQNQQT | 394 | 1.934 | 3.823 | 0.962 | 0.496 |
| GILLATPSGQNQQT | 395 | 1.931 | 8.205 | 1.341 | 0.288 |
| GSGQNAGSFPNQQT | 396 | 1.928 | 12.575 | 1.091 | 0.286 |
| GSRDGHTVGQNQQT | 397 | 1.928 | 7.089 | 0.495 | 0.661 |
| GSLLISTSGQNQQT | 398 | 1.919 | 5.763 | 1.488 | 0.808 |
| GSGAMPSHGQNQQT | 399 | 1.915 | 0.000 | 1.142 | 0.912 |
| GALVSPISGQNQQT | 400 | 1.912 | 1.051 | 0.640 | 0.347 |
| GSLSSHGVGQNQQT | 401 | 1.911 | 7.498 | 1.218 | 0.804 |
| GSGQNQQASLAMRT | 402 | 1.910 | 3.577 | 2.066 | 1.638 |
| GPGLGSHSGQNQQT | 403 | 1.906 | 14.563 | 0.880 | 1.195 |
| GHDSQHKSGQNQQT | 404 | 1.904 | 6.988 | 1.154 | 0.869 |
| GSGLTLSATQNQQT | 405 | 1.901 | 0.193 | 0.708 | 0.340 |
| GSGQVVAHVGNQQT | 406 | 1.901 | 0.833 | 0.800 | 0.321 |
| GSGLRTMTTQNQQT | 407 | 1.900 | 8.939 | 0.838 | 0.594 |
| GSGQVGRLLQNQQT | 408 | 1.899 | 1.762 | 0.773 | 0.748 |
| GSGQLSHQSVNQQT | 409 | 1.898 | 4.032 | 0.720 | 0.695 |
| GSGDRYQTLQNQQT | 410 | 1.897 | 1.075 | 0.645 | 0.318 |
| GSGQNQQLKSSAQT | 411 | 1.891 | 1.197 | 0.908 | 0.716 |
| GSGQNQYSIPVAQT | 412 | 1.891 | 1.194 | 0.511 | 0.297 |
| GSGERLHLTQNQQT | 413 | 1.885 | 1.456 | 0.387 | 0.245 |
| GSGHNQQVRTAPNT | 414 | 1.885 | 1.022 | 1.006 | 0.580 |
| GGLSHVMSGQNQQT | 415 | 1.875 | 0.403 | 0.885 | 0.378 |
| GSGQSHRDVLNQQT | 416 | 1.872 | 15.082 | 0.138 | 0.280 |
| GSGQNLAGRMDQQT | 417 | 1.864 | 0.085 | 0.362 | 0.295 |
| GSGQNQQTNRGNPM | 418 | 1.860 | 3.402 | 1.349 | 1.098 |
| GSGQSYQRDHNQQT | 419 | 1.859 | 8.013 | 0.779 | 0.323 |
| GSLLSAGMGQNQHT | 420 | 1.856 | 6.168 | 0.589 | 0.342 |
| GSGQNQQTAIYRNI | 421 | 1.854 | 2.207 | 0.818 | 1.437 |
| GSGQNQQTSGTTNC | 422 | 1.854 | 8.161 | 1.040 | 0.806 |
| GMTSHSVSGQNQQT | 423 | 1.850 | 2.732 | 0.220 | 0.154 |
| GSSQSTGYQPNQQT | 424 | 1.847 | 3.388 | 0.522 | 0.577 |
| GSLKPTTLGQNQQT | 425 | 1.840 | 0.476 | 0.175 | 0.220 |
| GRMFSLGSGQNQQT | 426 | 1.836 | 8.429 | 1.630 | 1.174 |
| GSGQNQQTALGVKC | 427 | 1.835 | 1.343 | 1.378 | 1.014 |
| GAMVSHSSGQNQQT | 428 | 1.833 | 8.999 | 0.739 | 0.868 |
| GSGQNQQRNSDSVT | 429 | 1.829 | 0.000 | 1.238 | 0.842 |
| GSGQSMTLHLNQQT | 430 | 1.827 | 0.991 | 0.721 | 0.248 |
| GSGQVHQAEVNQQT | 431 | 1.825 | 0.152 | 0.436 | 0.287 |
| GSGQNQSQNHLQQT | 432 | 1.825 | 0.600 | 1.063 | 0.772 |
| GSLLTTASGQNQQT | 433 | 1.822 | 0.780 | 0.938 | 0.635 |
| GSGLIRTAAQNQQT | 434 | 1.822 | 8.339 | 0.808 | 0.998 |
| GSGQNQQTVSRQST | 435 | 1.820 | 0.472 | 1.330 | 0.796 |
| GSGQYANHGINQQT | 436 | 1.820 | 5.717 | 0.906 | 0.701 |
| GSRSTGPSGQNQQT | 437 | 1.819 | 2.479 | 0.440 | 0.466 |
| GRGVQQKLQQNQQT | 438 | 1.817 | 0.000 | 1.974 | 0.823 |
| GSGQNQQVHLSTGT | 439 | 1.811 | 0.266 | 1.011 | 0.455 |
| GSGQNQQLSAKSST | 440 | 1.809 | 1.567 | 1.224 | 1.115 |
| GSGYKAARPQNQQT | 441 | 1.803 | 0.000 | 1.418 | 0.337 |
| GSAGISPSGQNQQT | 442 | 1.797 | 1.812 | 0.784 | 0.622 |
| GSGQNRAHAFLQQT | 443 | 1.795 | 0.000 | 1.200 | 1.271 |
| GSGLSGITMQNQQT | 444 | 1.792 | 14.796 | 0.862 | 0.496 |
| GPGSAHSSGQNQQT | 445 | 1.785 | 4.392 | 1.099 | 0.872 |
| GSSHTQALGQNQQT | 446 | 1.784 | 0.143 | 0.882 | 0.874 |
| GSGVHGVSSQNQQT | 447 | 1.781 | 4.519 | 1.504 | 0.951 |
| GSSGRDMGGQNQQT | 448 | 1.778 | 2.177 | 1.052 | 0.595 |
| GERAFPTSGQNQQT | 449 | 1.775 | 6.515 | 0.972 | 0.362 |
| GGRIVSLSGQNQQT | 450 | 1.766 | 4.936 | 1.161 | 0.847 |
| GSGQNSYSHTSQQT | 451 | 1.765 | 2.262 | 1.130 | 0.658 |
| GLGYPGSSGQNQQT | 452 | 1.763 | 7.090 | 0.929 | 0.577 |
| GSGPQSHTGQNQQT | 453 | 1.757 | 9.490 | 0.958 | 0.447 |
| GSGQNQQLSRDAST | 454 | 1.754 | 3.716 | 1.877 | 0.611 |
| GSGQILHSVPNQQT | 455 | 1.752 | 1.316 | 0.398 | 0.240 |
| GSGFHTDSRQNQQT | 456 | 1.748 | 4.384 | 7.344 | 0.575 |
| GSGQSHSLATNQQT | 457 | 1.745 | 2.711 | 1.021 | 0.343 |
| GSGQNQQTLSKPWT | 458 | 1.743 | 0.253 | 0.845 | 0.733 |
| GSGHAAISQQNQQT | 459 | 1.742 | 2.373 | 1.211 | 0.520 |
| GSGQNQQQIGGNST | 460 | 1.741 | 6.169 | 0.877 | 0.576 |
| GGGPMAGSGQNQQT | 461 | 1.735 | 2.815 | 1.049 | 0.372 |
| GMRMEYQSGQNQQT | 462 | 1.729 | 3.695 | 0.644 | 0.632 |
| GSGQNQQGTLLHQT | 463 | 1.728 | 2.065 | 1.347 | 1.303 |
| GSGQNQRSSGGVQT | 464 | 1.723 | 2.056 | 1.805 | 1.165 |
| GSGQNQRGALATQT | 465 | 1.722 | 1.117 | 0.899 | 0.891 |
| GSGTVHAATQNQQT | 466 | 1.721 | 1.676 | 0.563 | 0.476 |
| GSRMTQQFGQNQQT | 467 | 1.720 | 25.798 | 1.233 | 0.976 |
| GSSSPGASGQNQQT | 468 | 1.717 | 1.244 | 1.378 | 0.660 |
| GHPSPHVSGQNQQT | 469 | 1.713 | 0.416 | 0.551 | 0.488 |
| GSGSHHASRQNQQT | 470 | 1.712 | 0.451 | 3.073 | 0.584 |
| GAVGHSYSGQNQQT | 471 | 1.706 | 0.808 | 0.306 | 0.536 |
| GSRSQYDIGQNQQT | 472 | 1.706 | 0.112 | 0.528 | 0.193 |
| GSGQGPQERGNQQT | 473 | 1.702 | 1.269 | 0.846 | 0.313 |
| GSIAHVGTGQNQQT | 474 | 1.696 | 1.264 | 0.837 | 1.045 |
| GSGQNQQKQNHGNT | 475 | 1.695 | 5.349 | 1.538 | 1.340 |
| GSGQNQQALGSQRT | 476 | 1.695 | 1.934 | 1.419 | 0.562 |
| GSGAITHMPQNQQT | 477 | 1.695 | 1.681 | 0.647 | 0.411 |
| GSGQRNPLLLNQQT | 478 | 1.693 | 0.144 | 0.662 | 0.740 |
| GSSGIPVSHQNQQT | 479 | 1.690 | 3.384 | 0.820 | 0.333 |
| GVHSLTPSGQNQQT | 480 | 1.687 | 4.104 | 0.475 | 0.215 |
| GVIVLHGSGQNQQT | 481 | 1.682 | 14.166 | 1.074 | 1.098 |
| GGTRVVDSGQNQQT | 482 | 1.676 | 9.735 | 0.676 | 0.370 |
| GSGGVTYQSQNQQT | 483 | 1.673 | 7.283 | 0.649 | 0.181 |
| GSGQNQAGHGPGQT | 484 | 1.670 | 2.861 | 1.040 | 0.887 |
| GSGQLVTSGPNQQT | 485 | 1.669 | 5.271 | 0.964 | 0.433 |
| GSGIAAQRTQNQQT | 486 | 1.665 | 2.691 | 1.062 | 0.754 |
| GSTPAGVGGQNQQT | 487 | 1.663 | 2.733 | 0.593 | 0.477 |
| GSGQNQQTSTGVHS | 488 | 1.660 | 8.271 | 1.039 | 1.075 |
| GSGQIRQLVDNQQT | 489 | 1.657 | 5.529 | 0.314 | 0.272 |
| GSLIGMQSGQNQQT | 490 | 1.656 | 6.783 | 0.797 | 0.392 |
| GSGQIKGKMDNQQT | 491 | 1.654 | 2.601 | 1.065 | 1.012 |
| GSGSDMSSWQNQQT | 492 | 1.651 | 0.175 | 0.281 | 0.303 |
| GRGQNQQHTGLATT | 493 | 1.650 | 6.174 | 1.134 | 0.691 |
| GSGQNQQTLYSSNT | 494 | 1.642 | 1.044 | 0.664 | 0.368 |
| GSGQTQVLKSNQQT | 495 | 1.640 | 3.031 | 1.599 | 0.975 |
| GSRTLSNVGQNQQT | 496 | 1.640 | 3.219 | 0.617 | 0.542 |
| GSGVQHSLPQNQQT | 497 | 1.639 | 0.764 | 0.440 | 0.387 |
| GNYLHQASGQNQQT | 498 | 1.635 | 1.454 | 0.816 | 0.181 |
| GSGGTSVHQQNQQT | 499 | 1.629 | 0.000 | 0.585 | 0.195 |
| GMDHSRPSGQNQQT | 500 | 1.627 | 3.976 | 0.918 | 0.648 |
| GSGQNQQSMGTFTT | 501 | 1.625 | 0.000 | 1.792 | 0.399 |
| GSGQNQQTPLRPPT | 502 | 1.624 | 0.352 | 0.874 | 0.472 |
| GSGQNQHHSVSQQT | 503 | 1.623 | 3.700 | 0.605 | 0.334 |
| GSGQLRSLSTNQQT | 504 | 1.622 | 6.855 | 1.310 | 0.382 |
| GSGSPRQLSQNQQT | 505 | 1.621 | 0.873 | 0.520 | 0.273 |
| GSGQNQQTTASSHT | 506 | 1.618 | 7.404 | 0.745 | 0.678 |
| GRGQVVSTHQNQQT | 507 | 1.607 | 3.318 | 0.931 | 0.561 |
| GSAQVSMVGQNQQT | 508 | 1.601 | 1.332 | 0.500 | 0.285 |
| GSSTLVTIGKNQQT | 509 | 1.592 | 4.316 | 0.917 | 0.819 |
| GFAHQASSGQNQQT | 510 | 1.587 | 1.852 | 1.638 | 1.080 |
| GSGQPVLSISNQQT | 511 | 1.586 | 2.695 | 0.390 | 0.282 |
| GSGQSHRSELNQQT | 512 | 1.585 | 11.974 | 0.668 | 0.256 |
| GSSVGSPIGQNQQT | 513 | 1.584 | 3.574 | 1.059 | 0.706 |
| GSGMPIRNVQNQQT | 514 | 1.584 | 0.138 | 0.684 | 0.631 |
| GSSTRVDSGQNQQT | 515 | 1.584 | 2.774 | 0.704 | 0.660 |
| GSGQNQQTAMRSTT | 516 | 1.581 | 2.588 | 0.656 | 0.665 |
| GSGQNQQHSSSHLT | 517 | 1.581 | 2.782 | 1.091 | 0.859 |
| GSRNGHAVGQNQQT | 518 | 1.574 | 2.688 | 0.434 | 0.939 |
| GLGAYQSSGQNQQT | 519 | 1.574 | 0.696 | 1.407 | 0.688 |
| GPGLSGHSGQNQQT | 520 | 1.571 | 1.603 | 1.154 | 1.297 |
| GSTGIVSSGQNQQT | 521 | 1.570 | 0.927 | 2.141 | 1.046 |
| GSRTTQVIGQNQQT | 522 | 1.570 | 1.838 | 0.773 | 0.564 |
| GSGLLHRAQQNQQT | 523 | 1.569 | 0.724 | 1.583 | 0.646 |
| GSGQNAQQAAAQQT | 524 | 1.568 | 4.239 | 0.937 | 0.604 |
| GSGQNQQSALRTQT | 525 | 1.568 | 1.913 | 1.581 | 1.421 |
| GSGFLSDTRQNQQT | 526 | 1.566 | 45.953 | 0.473 | 0.575 |
| GSGLLYHDQQNQQT | 527 | 1.565 | 2.760 | 0.405 | 0.107 |
| GSGQNQHYSLHKQT | 528 | 1.563 | 3.399 | 1.485 | 1.273 |
| GSGHSPLPQQNQQT | 529 | 1.562 | 0.556 | 0.387 | 0.247 |
| GNGHSMRPNQNQQT | 530 | 1.560 | 2.341 | 0.693 | 0.376 |
| GSGLKWSTLQNQQT | 531 | 1.556 | 0.000 | 1.134 | 2.442 |
| GSGQMGRQAVNQQT | 532 | 1.554 | 1.529 | 0.535 | 0.411 |
| GSGQNQQTSGVLTL | 533 | 1.553 | 0.000 | 1.104 | 0.782 |
| GSGQNQQALHNPHT | 534 | 1.553 | 0.664 | 0.638 | 0.213 |
| GSGQNQQVIPNSKT | 535 | 1.548 | 1.036 | 0.844 | 0.376 |
| GSPLQDRVGQNQQT | 536 | 1.548 | 0.753 | 0.469 | 0.391 |
| GSGQNQYSSTNPQT | 537 | 1.542 | 2.251 | 0.544 | 0.535 |
| GAMTVTISGQNQQT | 538 | 1.542 | 6.249 | 0.443 | 0.257 |
| GSGQNQQLQTLIRT | 539 | 1.538 | 1.425 | 0.813 | 0.514 |
| GSGLRQTSQQNQQT | 540 | 1.537 | 2.067 | 0.978 | 0.705 |
| GSGQNQQTGLRQQT | 541 | 1.533 | 2.120 | 1.217 | 1.103 |
| GSGQTRQMKDNQQT | 542 | 1.530 | 11.079 | 0.841 | 0.214 |
| GSGQNHGLQSGQQT | 543 | 1.530 | 4.960 | 0.938 | 0.779 |
| GSGQSHRQPENQQT | 544 | 1.529 | 2.153 | 0.209 | 0.159 |
| GSGQDRHIVQNQQT | 545 | 1.527 | 11.068 | 0.285 | 0.162 |
| GSGQNQQLPHSNLT | 546 | 1.521 | 1.838 | 0.442 | 0.283 |
| GSGQLSVPYDNQQT | 547 | 1.521 | 0.000 | 0.622 | 0.111 |
| GSGRNPQTQPLQQT | 548 | 1.519 | 0.040 | 0.733 | 0.573 |
| GSGQPYSTGLNQQT | 549 | 1.519 | 1.403 | 0.612 | 0.376 |
| GSGQNQQTHGGLRD | 550 | 1.519 | 6.487 | 1.913 | 1.298 |
| GAYGMVSSGQNQQT | 551 | 1.518 | 3.469 | 0.732 | 0.773 |
| GSGIQSSYSQNQQT | 552 | 1.517 | 15.978 | 1.032 | 0.684 |
| GPRLSDQSGQNQQT | 553 | 1.511 | 0.364 | 0.640 | 0.579 |
| GSGQNQQTHPSPCT | 554 | 1.510 | 1.003 | 1.120 | 0.546 |
| GSGQSFQMHTNQQT | 555 | 1.504 | 9.770 | 0.503 | 0.325 |
| GSGQNQQTGNPKHT | 556 | 1.504 | 5.973 | 1.391 | 1.139 |
| GFSSAVHSGQNQQT | 557 | 1.502 | 1.234 | 0.218 | 0.210 |
| GSGQNQQTSMSNAT | 558 | 1.501 | 6.766 | 1.605 | 0.745 |
| GSGQDMKQHHNQQT | 559 | 1.501 | 1.638 | 0.358 | 0.239 |
| GLRLSTPSGQNQQT | 560 | 1.498 | 4.334 | 0.804 | 0.522 |
| GSGQNQQTSVYMNT | 561 | 1.498 | 0.613 | 0.640 | 0.983 |
| GSGQNQYSQSSMQT | 562 | 1.494 | 4.278 | 0.375 | 0.309 |
| GSGQNQQSMADHTT | 563 | 1.494 | 1.728 | 0.428 | 0.215 |
| GWERSFVSGQNQQT | 564 | 1.492 | 0.943 | 0.490 | 0.538 |
| GLLAGKSSGQNQQT | 565 | 1.491 | 2.981 | 0.999 | 0.946 |
| GKSFVPQSGQNQQT | 566 | 1.489 | 2.502 | 1.798 | 0.430 |
| GSGQMQSAGSNQQT | 567 | 1.482 | 0.116 | 1.034 | 1.128 |
| GSDQNQRLTSSMQT | 568 | 1.479 | 0.164 | 0.875 | 0.670 |
| GESRAVLSGQNQQT | 569 | 1.476 | 0.938 | 0.789 | 0.368 |
| GSVFGVPSGQNQQT | 570 | 1.474 | 1.248 | 0.685 | 0.213 |
| GSGLPDRNLQNQQT | 571 | 1.471 | 7.306 | 1.136 | 0.611 |
| GSGTHNSAIQNQQT | 572 | 1.469 | 0.570 | 0.762 | 0.574 |
| GSGMIIASMQNQQT | 573 | 1.469 | 6.722 | 1.135 | 0.415 |
| GGITWTDSGQNQQT | 574 | 1.462 | 4.535 | 1.472 | 0.468 |
| GSGQNQQASGRQQT | 575 | 1.458 | 3.179 | 0.943 | 0.991 |
| GSGQNQQPHLKSLT | 576 | 1.457 | 5.016 | 1.096 | 0.740 |
| GPPQHMTSGQNQQT | 577 | 1.457 | 1.547 | 0.509 | 0.677 |
| GSGQNQQASLPSRT | 578 | 1.456 | 0.389 | 0.930 | 0.673 |
| GSGQIVSTQTNQQT | 579 | 1.456 | 1.103 | 0.453 | 0.512 |
| GSGKGHSAGQNQQT | 580 | 1.453 | 0.936 | 1.035 | 1.173 |
| GSGQNTRLQLGQQT | 581 | 1.452 | 1.747 | 0.181 | 0.234 |
| GSVGSRPVGQNQQT | 582 | 1.442 | 11.363 | 1.182 | 0.716 |
| GSSHTLALGQNQQT | 583 | 1.441 | 7.071 | 0.851 | 0.406 |
| GMYEYSQSGQNQQT | 584 | 1.438 | 0.000 | 1.410 | 0.448 |
| GNGQNQQHSILHGT | 585 | 1.435 | 0.000 | 0.777 | 0.415 |
| GSGYNQPHLQNQQT | 586 | 1.435 | 4.512 | 0.711 | 0.395 |
| GPLVNASSGQNQQT | 587 | 1.434 | 5.239 | 0.831 | 0.343 |
| GSGQNQQVLTTART | 588 | 1.434 | 4.142 | 1.071 | 0.948 |
| GSGQNQHSVHNDQT | 589 | 1.428 | 0.000 | 0.521 | 0.515 |
| GAGLIMHSGQNQQT | 590 | 1.425 | 1.408 | 0.565 | 0.511 |
| GMGRHSASGQNQQT | 591 | 1.417 | 6.500 | 0.470 | 0.389 |
| GSHSQSGHGQNQQT | 592 | 1.413 | 1.240 | 0.696 | 0.318 |
| GSSTTIVSGQNHQT | 593 | 1.411 | 0.000 | 0.993 | 0.672 |
| GRHLVTASGQNQQT | 594 | 1.411 | 2.885 | 0.648 | 0.404 |
| GSGQNQQHANLNQT | 595 | 1.410 | 0.094 | 0.416 | 0.544 |
| GSGSTHSKAQNQQT | 596 | 1.410 | 0.515 | 0.921 | 0.801 |
| GSGQNKQMLSGNTT | 597 | 1.410 | 2.219 | 1.074 | 0.404 |
| GSGQVHNPTQNQQT | 598 | 1.410 | 2.488 | 1.021 | 0.542 |
| GSGQNQQIPHVHQT | 599 | 1.409 | 0.768 | 0.576 | 0.218 |
| GSLHAGLSGQNQQT | 600 | 1.408 | 1.739 | 1.286 | 0.936 |
| GPAQHGTSGQNQQT | 601 | 1.407 | 0.866 | 1.030 | 0.615 |
| GEKAVTSSGQNQQT | 602 | 1.402 | 0.998 | 0.558 | 0.327 |
| GSGQNQQTMANGQR | 603 | 1.394 | 0.216 | 1.169 | 1.230 |
| GSGSPHSKDQNQQT | 604 | 1.394 | 0.000 | 2.041 | 4.680 |
| GSFSMGYGGQNQQT | 605 | 1.393 | 18.476 | 1.908 | 1.030 |
| GSGTHLVSLQNQQT | 606 | 1.392 | 0.000 | 0.715 | 1.167 |
| GSGQMQPHVQNQQT | 607 | 1.389 | 9.381 | 0.387 | 0.153 |
| GSGQNQQVAGLNNT | 608 | 1.386 | 3.218 | 0.449 | 0.492 |
| GSSQNQQHDMRLRT | 609 | 1.386 | 2.645 | 0.669 | 0.552 |
| GPASLPISGQNQQT | 610 | 1.386 | 9.008 | 0.312 | 0.155 |
| GSGQNQQPPLATRT | 611 | 1.386 | 2.295 | 0.593 | 0.287 |
| GSSRVPVSGQNQQT | 612 | 1.385 | 13.191 | 0.870 | 0.485 |
| GSGQNQQTNLGHTT | 613 | 1.383 | 1.523 | 1.343 | 1.281 |
| GSGQNQQLVSRVQT | 614 | 1.381 | 1.195 | 0.656 | 0.466 |
| GPNSYPVSGQKQQT | 615 | 1.381 | 4.040 | 0.736 | 0.834 |
| GHAHYQASGQNQQT | 616 | 1.377 | 7.299 | 0.803 | 0.745 |
| GSGQALLSTGNQQT | 617 | 1.377 | 0.847 | 0.536 | 0.370 |
| GSGQLPRQMTNQQT | 618 | 1.376 | 3.550 | 0.400 | 0.562 |
| GSGFPKSTEQNQQT | 619 | 1.376 | 2.058 | 0.610 | 0.194 |
| GSRETSLSGQNQQT | 620 | 1.373 | 5.193 | 1.364 | 0.203 |
| GSGQNQQGTGVSHT | 621 | 1.371 | 4.295 | 1.417 | 0.749 |
| GSRTVPVYGQNQQT | 622 | 1.371 | 0.363 | 1.226 | 0.969 |
| GSNAQSAHGQNQQT | 623 | 1.371 | 0.888 | 0.976 | 0.245 |
| GAFHLAASGQNQQT | 624 | 1.369 | 18.165 | 0.994 | 0.775 |
| GSGQYRSSSDNQQT | 625 | 1.369 | 6.209 | 0.681 | 0.409 |
| GSGQVYISTPNQQT | 626 | 1.367 | 0.000 | 0.859 | 0.282 |
| GSGVSTQLLQNQQT | 627 | 1.367 | 2.467 | 0.928 | 0.509 |
| GSGQLGLSVTNQQT | 628 | 1.364 | 6.906 | 1.395 | 0.376 |
| GSGSNMRLSQNQQT | 629 | 1.363 | 0.588 | 0.962 | 0.730 |
| GSGQNLHSGLPQQT | 630 | 1.363 | 1.594 | 1.054 | 0.592 |
| GSSHTLALGQNKQT | 631 | 1.362 | 2.160 | 0.838 | 0.643 |
| GSGQNQHSLPAHRT | 632 | 1.361 | 0.700 | 0.911 | 0.742 |
| GSGQNQGTVYPNQT | 633 | 1.358 | 7.648 | 0.835 | 0.815 |
| GSGQNQQPSLRQST | 634 | 1.356 | 2.905 | 1.315 | 0.554 |
| GSGQNARLKDNQQT | 635 | 1.354 | 2.395 | 0.580 | 0.938 |
| GHAGSTGSGQNQQT | 636 | 1.352 | 2.829 | 1.332 | 1.233 |
| GSGQALSSSGNQQT | 637 | 1.351 | 6.860 | 0.894 | 0.931 |
| GSGASESHRQNQQT | 638 | 1.350 | 0.850 | 0.325 | 0.313 |
| GVGVITSSGQNQQT | 639 | 1.348 | 0.918 | 1.296 | 0.777 |
| GSLYGQSLGQNQQT | 640 | 1.348 | 11.248 | 0.894 | 0.843 |
| GSGQMSDVHGNQQT | 641 | 1.346 | 7.172 | 0.408 | 0.548 |
| GSGQNQQHSSKATT | 642 | 1.345 | 12.248 | 1.350 | 1.401 |
| GSGQNQQTSVSQQT | 643 | 1.342 | 1.614 | 1.030 | 0.913 |
| GSGQKMWKLDNQQT | 644 | 1.341 | 0.000 | 0.990 | 1.418 |
| GSGQNVSMQVNQQT | 645 | 1.341 | 0.000 | 0.357 | 0.251 |
| GSGQNQRATLSNQT | 646 | 1.339 | 1.084 | 0.947 | 0.723 |
| GSGQASSKSANQQT | 647 | 1.339 | 1.138 | 0.500 | 0.175 |
| GSGKNQTPIPKGQT | 648 | 1.339 | 5.077 | 1.306 | 1.154 |
| GSGQNQQTRQEGST | 649 | 1.339 | 0.000 | 0.645 | 0.718 |
| GASSLATSGQNQQT | 650 | 1.337 | 0.703 | 0.423 | 0.217 |
| GSGQRGSLTENQQT | 651 | 1.337 | 2.482 | 0.300 | 0.567 |
| GSEQTRQRGQNQQT | 652 | 1.333 | 2.172 | 0.574 | 0.815 |
| GSGQNQQTLTASKE | 653 | 1.333 | 1.152 | 0.981 | 1.172 |
| GSGTSGKTGKNQQT | 654 | 1.333 | 4.033 | 0.358 | 0.676 |
| GQLVTFTSGQNQQT | 655 | 1.331 | 11.282 | 0.819 | 0.294 |
| GSGQNQQSANKILT | 656 | 1.331 | 3.789 | 0.894 | 1.236 |
| GSGQNQQHHSSHTT | 657 | 1.328 | 2.158 | 0.957 | 0.452 |
| GSGQNQKGMQPNQT | 658 | 1.326 | 3.139 | 0.775 | 1.059 |
| GSGQLVSGLYNQQT | 659 | 1.325 | 0.000 | 0.842 | 0.733 |
| GSSVGVPSGQNQQT | 660 | 1.322 | 4.867 | 0.336 | 1.157 |
| GSGQNQQWDSRRQT | 661 | 1.321 | 0.531 | 1.059 | 0.825 |
| GSEQTRQSGQNQQT | 662 | 1.321 | 0.514 | 0.734 | 0.900 |
| GSGIGSHIPQNQQT | 663 | 1.319 | 0.173 | 0.822 | 0.597 |
| GSGQNQRLHGVDQT | 664 | 1.318 | 4.655 | 0.459 | 0.341 |
| GEVSRVLSGQNQQT | 665 | 1.318 | 0.437 | 1.150 | 0.440 |
| GSGQNQQKVSPLLT | 666 | 1.314 | 1.602 | 0.755 | 0.806 |
| GSGLALERSQNQQT | 667 | 1.311 | 0.486 | 0.618 | 0.096 |
| GPDRIGSSGQNQQT | 668 | 1.308 | 0.426 | 0.654 | 0.342 |
| GSGQNQDHQNKQQT | 669 | 1.308 | 1.470 | 0.510 | 0.761 |
| GSGQNQQTALYNNT | 670 | 1.307 | 0.862 | 0.660 | 0.726 |
| GSGAVHLTAQNQQT | 671 | 1.306 | 1.668 | 0.541 | 0.466 |
| GSLVSTQSGQNQQT | 672 | 1.305 | 1.293 | 1.282 | 0.650 |
| GSGVSARMVQNQQT | 673 | 1.299 | 0.624 | 0.870 | 0.697 |
| GSGQTRMPLANQQT | 674 | 1.296 | 0.790 | 0.447 | 0.273 |
| GSGISSRNMQNQQT | 675 | 1.291 | 6.328 | 1.671 | 0.560 |
| GSGEKVHSGQNQQT | 676 | 1.289 | 0.062 | 0.862 | 0.671 |
| GSGQNQQKLSSMST | 677 | 1.286 | 1.586 | 1.160 | 1.052 |
| GSGQNQQTGQHMRV | 678 | 1.286 | 4.161 | 1.839 | 1.635 |
| GSGMIHTTAQNQQT | 679 | 1.285 | 0.105 | 0.678 | 0.276 |
| GSGQNWPALKGQQT | 680 | 1.284 | 2.031 | 1.101 | 1.222 |
| GASHMSISGQNQQT | 681 | 1.284 | 0.462 | 0.404 | 0.374 |
| GSDQNQQLGYSKQT | 682 | 1.283 | 0.000 | 0.853 | 0.660 |
| GIPSIRESGQNQQT | 683 | 1.282 | 0.166 | 0.484 | 0.254 |
| GSGIPSVKFQNQQT | 684 | 1.281 | 0.061 | 0.364 | 0.561 |
| GSGQNQQTSVSQNV | 685 | 1.281 | 0.750 | 0.788 | 0.715 |
| GSGQNQQIGESRMT | 686 | 1.279 | 0.103 | 0.890 | 0.453 |
| GSGSSSMSFQNQQT | 687 | 1.279 | 0.540 | 0.466 | 0.095 |
| GSGQKQERAVSKQT | 688 | 1.277 | 0.000 | 1.174 | 0.732 |
| GCTTRLNSGQNQQT | 689 | 1.276 | 0.000 | 0.184 | 0.618 |
| GSGQNQQIISTKIT | 690 | 1.275 | 0.000 | 0.951 | 0.710 |
| GSGQNQQKSLNGNT | 691 | 1.275 | 8.573 | 0.586 | 0.851 |
| GSGIPAPRLQNQQT | 692 | 1.273 | 4.162 | 0.583 | 0.396 |
| GSGQIRESMGNQQT | 693 | 1.270 | 1.676 | 0.833 | 0.523 |
| GSGQNSGVHFNQQT | 694 | 1.268 | 0.587 | 0.871 | 0.377 |
| GSGQNIGHSLPQQT | 695 | 1.264 | 6.183 | 0.740 | 0.478 |
| GSGERSISVQNQQT | 696 | 1.264 | 1.619 | 0.598 | 0.173 |
| GSGLKPNVLQNQQT | 697 | 1.263 | 0.975 | 0.701 | 0.268 |
| GSGQVAYAQGNQQT | 698 | 1.259 | 1.309 | 0.734 | 0.313 |
| GSGQSSYGSGNQQT | 699 | 1.257 | 1.686 | 1.161 | 0.456 |
| GSGQNQAMTHGDQT | 700 | 1.257 | 1.878 | 0.357 | 0.259 |
| GSGQNQALVSMGQT | 701 | 1.255 | 1.876 | 0.987 | 0.560 |
| GSGQNPSFMRGQQT | 702 | 1.252 | 1.454 | 1.293 | 1.094 |
| GSGQNQQSHLRTNT | 703 | 1.251 | 4.583 | 1.022 | 0.718 |
| GYTRLETSGQNQQT | 704 | 1.250 | 1.323 | 0.841 | 0.297 |
| GSGQSYDMRGNQQT | 705 | 1.248 | 0.567 | 0.588 | 0.368 |
| GSRTTQDIGQNQQT | 706 | 1.247 | 0.000 | 0.685 | 0.280 |
| GSGHPYKAAQNQQT | 707 | 1.246 | 0.000 | 0.872 | 0.507 |
| GRLSNAHGGQNQQT | 708 | 1.245 | 0.839 | 1.036 | 0.725 |
| GSGQNQRAVLNDQT | 709 | 1.242 | 3.023 | 0.556 | 0.259 |
| GGSHTYGGGQNQQT | 710 | 1.241 | 13.065 | 0.982 | 0.730 |
| GSSVNSMIGQNQQT | 711 | 1.239 | 0.000 | 0.976 | 0.580 |
| GNSSMMGSGQNQQT | 712 | 1.239 | 3.856 | 0.656 | 0.364 |
| GNRDRPSSGQNQQT | 713 | 1.239 | 3.947 | 0.298 | 0.178 |
| GSGNMHASRQNQQT | 714 | 1.238 | 3.878 | 0.782 | 0.687 |
| GFIFPKVSGQNQQT | 715 | 1.237 | 0.000 | 1.764 | 0.692 |
| GSGQNQQLKNSTST | 716 | 1.235 | 1.703 | 1.063 | 0.538 |
| GSGQNQQSQYMPRT | 717 | 1.234 | 0.401 | 0.549 | 0.520 |
| GSGQRMADIGNQQT | 718 | 1.233 | 2.539 | 0.352 | 0.427 |
| GSGQNQSHYPSQQT | 719 | 1.228 | 4.315 | 0.644 | 0.402 |
| GSDGKMHRGQNQQT | 720 | 1.227 | 0.000 | 1.826 | 0.776 |
| GSGSVGFIGQNQQT | 721 | 1.227 | 8.261 | 0.689 | 0.445 |
| GLHGMTLSGQNQQT | 722 | 1.226 | 3.552 | 0.470 | 0.338 |
| GSDQSKRGDSNQQT | 723 | 1.225 | 0.639 | 0.479 | 0.267 |
| GSLFLATGGQNQQT | 724 | 1.220 | 0.000 | 0.775 | 0.485 |
| GSGQNQQPSAFSKT | 725 | 1.220 | 4.906 | 1.309 | 0.754 |
| GSGQLPQSGLNQQT | 726 | 1.218 | 1.504 | 0.641 | 0.318 |
| GSGSKQNALQNQQT | 727 | 1.216 | 2.010 | 0.941 | 0.594 |
| GSGQRRELSQNQQT | 728 | 1.215 | 1.791 | 0.622 | 0.396 |
| GSGQREPKASNQQT | 729 | 1.214 | 2.793 | 0.399 | 0.520 |
| GSGQNQQHPSTQQT | 730 | 1.205 | 1.552 | 1.017 | 0.680 |
| GSQSTLGLGQNQQT | 731 | 1.204 | 3.246 | 0.594 | 0.400 |
| GSGQNQQMPGLSST | 732 | 1.204 | 1.887 | 0.234 | 0.181 |
| GSGQNQQTVGGKNL | 733 | 1.203 | 0.128 | 0.777 | 1.051 |
| GSSREFHSGQNQQT | 734 | 1.203 | 1.591 | 0.688 | 0.474 |
| GSGQNQQTVPSNLV | 735 | 1.201 | 0.791 | 0.434 | 0.281 |
| GSGQNAYSSQAQQT | 736 | 1.201 | 12.096 | 0.629 | 0.216 |
| GSGQNKDHSTRRQT | 737 | 1.197 | 0.000 | 0.384 | 0.477 |
| GQLGSVGSGQDQQT | 738 | 1.196 | 0.000 | 1.020 | 0.437 |
| GSGQHAAPGHNQQT | 739 | 1.195 | 5.999 | 0.600 | 0.199 |
| GSGQNQQTSQSPPT | 740 | 1.194 | 1.208 | 0.851 | 0.478 |
| GSGNYRDHEQNQQT | 741 | 1.193 | 7.389 | 0.287 | 0.222 |
| GSGQHSNQHVNQQT | 742 | 1.192 | 1.453 | 0.955 | 0.558 |
| GSGQTARNGINQQT | 743 | 1.192 | 2.030 | 1.002 | 0.472 |
| GSGQNQQHYGSQGT | 744 | 1.189 | 0.453 | 1.345 | 0.379 |
| GSGSPQASRQNQQT | 745 | 1.189 | 6.782 | 0.923 | 0.542 |
| GSGFSHSMGKNQQT | 746 | 1.188 | 9.809 | 1.381 | 0.611 |
| GSGQSHSLETNQQT | 747 | 1.188 | 1.319 | 0.520 | 0.363 |
| GTEQTRQSGQNQQT | 748 | 1.188 | 0.132 | 0.756 | 0.756 |
| GSGRHLASVQNQQT | 749 | 1.187 | 1.024 | 0.654 | 0.606 |
| GLGSKNHSGQNQQT | 750 | 1.187 | 5.046 | 0.825 | 0.224 |
| GSGQNQQTSHFPSA | 751 | 1.185 | 0.325 | 0.969 | 0.907 |
| GSGQLSGTPQNQQT | 752 | 1.185 | 1.382 | 1.025 | 0.643 |
| GSGQNQQAPHKKET | 753 | 1.180 | 0.598 | 0.994 | 0.689 |
| GSGQNQQTLRGSLE | 754 | 1.179 | 1.812 | 0.853 | 0.354 |
| GSIAMTSHGQNQQT | 755 | 1.178 | 1.435 | 0.551 | 0.438 |
| GSPGVSPSGQNQQT | 756 | 1.178 | 3.006 | 0.853 | 1.160 |
| GSGQNQQTGSSSRV | 757 | 1.176 | 0.580 | 0.995 | 1.128 |
| GSGQHLPLLGNQQT | 758 | 1.175 | 1.739 | 0.519 | 0.347 |
| GSDHSHRGGQNQQT | 759 | 1.174 | 0.504 | 0.818 | 0.331 |
| GSGIVTKLGQNQQT | 760 | 1.174 | 10.571 | 0.599 | 0.242 |
| GSGQDVTKTGNQQT | 761 | 1.173 | 4.523 | 0.531 | 0.035 |
| GSGQNQQSHGRIGT | 762 | 1.173 | 5.117 | 0.607 | 0.455 |
| GSGQNQQINHRSPT | 763 | 1.173 | 0.748 | 0.259 | 0.220 |
| GSGDDSRVGQNQQT | 764 | 1.172 | 0.191 | 0.466 | 0.156 |
| GSGQSTLKRINQQT | 765 | 1.168 | 13.442 | 0.534 | 1.184 |
| GSGSQHSKAQNQQT | 766 | 1.168 | 0.312 | 0.638 | 0.916 |
| GSGQNQQHASSNNT | 767 | 1.166 | 7.155 | 0.789 | 0.896 |
| GSRTYQVSGQNQQT | 768 | 1.164 | 1.853 | 0.638 | 0.641 |
| GSGQNQGLLSSPQT | 769 | 1.164 | 0.000 | 0.707 | 0.417 |
| GSGGGLQHNQNQQT | 770 | 1.163 | 4.098 | 1.137 | 0.778 |
| GSGQNQQTTAATRM | 771 | 1.163 | 3.925 | 0.947 | 1.005 |
| GSGQNQRASILVQT | 772 | 1.162 | 3.632 | 0.531 | 0.569 |
| GSGQNLGLLGAQQT | 773 | 1.161 | 1.458 | 0.524 | 0.226 |
| GSLDLGRSGQNQQT | 774 | 1.160 | 3.283 | 1.002 | 0.505 |
| GNSQVKVSGQNQQT | 775 | 1.158 | 4.930 | 1.422 | 0.728 |
| GSSGSHQYGQNQQT | 776 | 1.155 | 0.000 | 1.129 | 0.794 |
| GSGQNQQQRDGTLT | 777 | 1.152 | 0.387 | 0.760 | 0.730 |
| GRGQHVSVANNQQT | 778 | 1.152 | 1.896 | 1.032 | 0.589 |
| GDSSSRISGQNQQT | 779 | 1.151 | 3.787 | 0.916 | 0.348 |
| GSGQNQQHSLSSQT | 780 | 1.150 | 3.844 | 0.700 | 0.730 |
| GSLMDVHRGQNQQT | 781 | 1.150 | 0.387 | 1.009 | 0.238 |
| GSIQYQSSGQNQQT | 782 | 1.147 | 2.601 | 1.074 | 1.191 |
| GLGSKNPSGQNQQT | 783 | 1.147 | 1.629 | 1.184 | 0.424 |
| GSGQLVLTLQNQQT | 784 | 1.143 | 0.000 | 0.336 | 0.336 |
| GSGQNQQTSQPLPG | 785 | 1.141 | 0.080 | 0.748 | 0.530 |
| GSGQNQQNLGKLNT | 786 | 1.141 | 0.000 | 0.919 | 0.687 |
| GTTAHQPSGQNQQT | 787 | 1.138 | 0.211 | 0.726 | 0.275 |
| GSGQNRAQIGTQQT | 788 | 1.138 | 0.469 | 0.776 | 0.654 |
| GSGQYVHVSSNQQT | 789 | 1.137 | 1.803 | 0.739 | 0.366 |
| GSGQNQQTAHAFNI | 790 | 1.132 | 3.404 | 0.699 | 0.729 |
| GSGQNQRTMVATQT | 791 | 1.130 | 1.122 | 0.649 | 0.554 |
| GSGQNPIRGAMQQT | 792 | 1.126 | 1.327 | 1.296 | 0.427 |
| GSGYVITGSQNQQT | 793 | 1.125 | 6.271 | 0.971 | 0.248 |
| GRGPKQSNIQNQQT | 794 | 1.125 | 0.737 | 0.771 | 2.490 |
| GSGQNQQTMLGKPC | 795 | 1.125 | 0.047 | 1.090 | 0.992 |
| GSGQNQQVGSTVRT | 796 | 1.124 | 2.040 | 0.918 | 0.614 |
| GNVTTQKSGQNQQT | 797 | 1.122 | 2.546 | 1.215 | 0.922 |
| GSGNPVSHLQNQQT | 798 | 1.121 | 1.037 | 0.583 | 0.310 |
| GSLSHMESGQNQQT | 799 | 1.120 | 0.829 | 0.489 | 0.265 |
| GRAPTNLSGQNQQT | 800 | 1.118 | 0.687 | 0.757 | 0.169 |
| GSGQNQQTVMTARA | 801 | 1.117 | 1.535 | 0.995 | 0.843 |
| GSGMPASRLQNQQT | 802 | 1.117 | 1.689 | 0.790 | 0.372 |
| GVVRNHQSGQNQQT | 803 | 1.116 | 5.801 | 0.899 | 0.868 |
| GSGQNQHSVQVRQT | 804 | 1.116 | 1.909 | 0.782 | 0.916 |
| GSGQNTGHLTMQQT | 805 | 1.114 | 0.078 | 1.026 | 0.595 |
| GSGQNQQYAGKILT | 806 | 1.112 | 0.300 | 1.078 | 0.431 |
| GSGNPHVRNQNQQT | 807 | 1.112 | 0.873 | 0.732 | 0.755 |
| GSGQNGGSSNRQQT | 808 | 1.109 | 2.594 | 1.255 | 0.844 |
| GSGQRLSQGVNHQT | 809 | 1.108 | 3.394 | 0.931 | 1.141 |
| GSGQNAHAKEGQQT | 810 | 1.108 | 0.000 | 0.875 | 1.179 |
| GSSPAPNSGQNQQT | 811 | 1.106 | 2.229 | 0.719 | 0.368 |
| GLAHKTSSGQNQQT | 812 | 1.106 | 0.915 | 0.427 | 0.690 |
| GSGQNQQTPGAHKT | 813 | 1.105 | 3.827 | 0.957 | 0.277 |
| GSGQNQQSLSGSFT | 814 | 1.105 | 0.735 | 0.745 | 0.883 |
| GSGQNQQSTGTSRT | 815 | 1.103 | 4.054 | 1.209 | 0.935 |
| GSGQNQQTVQSNLV | 816 | 1.103 | 2.350 | 0.577 | 0.698 |
| GSGQNQQLGSRQCT | 817 | 1.102 | 0.183 | 0.987 | 0.407 |
| GSGQNQYLRLELQT | 818 | 1.101 | 0.000 | 0.416 | 0.839 |
| GSGQNQQTSPRLQT | 819 | 1.100 | 0.795 | 1.156 | 1.091 |
| GSGQNQQTTSSNMT | 820 | 1.099 | 0.569 | 0.638 | 0.698 |
| GTASTYNSGQNQQT | 821 | 1.099 | 2.560 | 0.250 | 0.625 |
| GSGQNQQTMPQHKI | 822 | 1.097 | 2.394 | 0.479 | 0.197 |
| GSGQSHLHTGNQQT | 823 | 1.096 | 2.584 | 0.721 | 0.295 |
| GVKGVGHSGQNQQT | 824 | 1.096 | 2.485 | 0.994 | 0.783 |
| GSGKVTKQSQNQQT | 825 | 1.095 | 0.000 | 0.928 | 1.035 |
| GSGQNQQTALEKSL | 826 | 1.092 | 0.000 | 0.625 | 0.702 |
| GSGYKDTYGQNQQT | 827 | 1.091 | 0.854 | 0.717 | 0.448 |
| GSGQNQQSGTFLST | 828 | 1.090 | 5.673 | 1.021 | 0.742 |
| GSGQNTGQHMMQQT | 829 | 1.090 | 1.058 | 1.147 | 0.917 |
| GSGKNQQRPGLDQT | 830 | 1.089 | 1.557 | 0.583 | 0.385 |
| GSGQSREISLNQQT | 831 | 1.088 | 6.954 | 0.594 | 0.282 |
| GTPTSPSSGQNQQT | 832 | 1.086 | 4.558 | 0.833 | 0.662 |
| GKPAGGLSGQNQQT | 833 | 1.085 | 2.805 | 0.708 | 0.739 |
| GSGQNHRSADMQQT | 834 | 1.084 | 12.001 | 0.417 | 0.212 |
| GSGQNQQTLPSLSL | 835 | 1.084 | 1.758 | 0.527 | 0.175 |
| GSPYMGATGQNQQT | 836 | 1.083 | 5.364 | 0.918 | 0.254 |
| GSGHAKAVGQNQQT | 837 | 1.081 | 4.357 | 0.703 | 0.824 |
| GHMKGVTSGQNQQT | 838 | 1.081 | 2.814 | 0.807 | 0.413 |
| GSGQNQKILTLDQT | 839 | 1.080 | 0.371 | 0.291 | 0.314 |
| GSGQNQQTKVGHSA | 840 | 1.079 | 1.256 | 0.669 | 1.019 |
| GIARTTISGQNQQT | 841 | 1.078 | 1.783 | 0.819 | 0.330 |
| GSGQNQQTSVGFRT | 842 | 1.077 | 3.737 | 0.648 | 0.534 |
| GSGQNQQTMIANIR | 843 | 1.076 | 0.000 | 0.379 | 0.458 |
| GDMTRSSSGQNQQT | 844 | 1.075 | 0.802 | 1.145 | 1.038 |
| GSGHMSDLRQNQQT | 845 | 1.073 | 4.291 | 0.555 | 0.328 |
| GRGAVMASGQNQQT | 846 | 1.072 | 0.923 | 0.783 | 0.605 |
| GSGQNQQLSGKSVT | 847 | 1.070 | 1.524 | 1.276 | 0.930 |
| GSHTLVVSGQNQQT | 848 | 1.069 | 1.535 | 0.671 | 0.748 |
| GSGPWSAGLQNQQT | 849 | 1.067 | 0.947 | 0.700 | 0.539 |
| GSGQHSPHALNQQT | 850 | 1.064 | 1.412 | 0.885 | 0.573 |
| GSGQNQQPNSGSMT | 851 | 1.064 | 0.925 | 0.588 | 0.339 |
| GSGLAHLGGQNQQT | 852 | 1.064 | 2.191 | 0.749 | 0.794 |
| GSSVRYEPKQNQQT | 853 | 1.063 | 1.564 | 0.450 | 0.501 |
| GSGQNQQARPLELT | 854 | 1.061 | 0.059 | 0.389 | 0.252 |
| GSGQPRSTGINQQT | 855 | 1.061 | 0.693 | 0.650 | 0.542 |
| GSGQNQANWVKVQT | 856 | 1.059 | 0.126 | 0.683 | 0.532 |
| GSGHLFQSGQNQQT | 857 | 1.057 | 0.615 | 0.751 | 0.386 |
| GSGQNRGISISQQT | 858 | 1.057 | 2.166 | 0.686 | 0.566 |
| GSGTHYDNRQNQQT | 859 | 1.054 | 0.072 | 0.612 | 0.486 |
| GSGQNQQTSTTPLP | 860 | 1.052 | 2.823 | 0.828 | 0.741 |
| GSGQVHASQVNQKT | 861 | 1.049 | 0.503 | 0.855 | 0.767 |
| GSSGHRESGQNQQT | 862 | 1.048 | 4.398 | 0.641 | 0.691 |
| GLSAEKSSGQNQQT | 863 | 1.047 | 7.203 | 0.629 | 0.303 |
| GSGQEHRSLANQQT | 864 | 1.046 | 0.000 | 0.507 | 0.344 |
| GSGQTVVRIANQQT | 865 | 1.046 | 4.156 | 0.661 | 0.390 |
| GSGQNVSSVHRQQT | 866 | 1.045 | 0.712 | 0.383 | 0.271 |
| GSGASRMSIQNQQT | 867 | 1.045 | 0.111 | 0.801 | 0.417 |
| GVAFIGSSGQNQQT | 868 | 1.043 | 0.000 | 0.744 | 0.648 |
| GSGQNQQTVPTRQT | 869 | 1.040 | 1.207 | 0.629 | 0.138 |
| GSGQAAKSSQNQQT | 870 | 1.036 | 0.681 | 0.778 | 0.737 |
| GSGQNQQVAIRTST | 871 | 1.035 | 2.447 | 0.963 | 0.370 |
| GSVHMQNAGQNQQT | 872 | 1.034 | 3.608 | 1.004 | 0.625 |
| GSGMRQAGVQNQQT | 873 | 1.032 | 0.811 | 0.736 | 0.775 |
| GSGQNQQVGGKTVT | 874 | 1.032 | 6.195 | 1.094 | 0.821 |
| GVHDMRVSGQNQQT | 875 | 1.032 | 8.083 | 1.171 | 0.818 |
| GSGQHVSVANNQQT | 876 | 1.029 | 5.734 | 0.974 | 0.577 |
| GSAAMSVRGQNQQT | 877 | 1.029 | 2.386 | 0.202 | 0.287 |
| GVSRGGPSGQNQQT | 878 | 1.028 | 1.611 | 0.750 | 0.591 |
| GSGQMVHTIGNQQT | 879 | 1.026 | 1.328 | 0.406 | 0.430 |
| GRGGSMAETQNQQT | 880 | 1.024 | 2.853 | 0.799 | 0.669 |
| GSGHTNPTRQNQQT | 881 | 1.021 | 0.688 | 0.726 | 0.807 |
| GSGEAARYEQNQQT | 882 | 1.020 | 0.000 | 0.107 | 0.125 |
| GSGQNERHLVLQQT | 883 | 1.019 | 5.354 | 0.416 | 0.150 |
| GSGQNQQSKQQVLT | 884 | 1.019 | 1.494 | 1.428 | 1.256 |
| GSGQARAHRGNQQT | 885 | 1.017 | 0.000 | 0.254 | 0.386 |
| GSGQNQQPLDTSRT | 886 | 1.015 | 0.775 | 0.491 | 0.376 |
| GSGQNQQLANMVTT | 887 | 1.014 | 1.739 | 1.253 | 0.987 |
| GSGQMKDLHRNQQT | 888 | 1.014 | 1.068 | 0.587 | 0.506 |
| GSGQNQHLSSFVQT | 889 | 1.013 | 0.110 | 1.090 | 0.364 |
| GSGQNQQPSSRVTT | 890 | 1.012 | 2.179 | 0.784 | 0.504 |
| GSGQNQQLAITLGT | 891 | 1.011 | 0.000 | 0.877 | 0.143 |
| GSGQNQQTVGNPAT | 892 | 1.008 | 3.014 | 0.856 | 0.395 |
| GSGQNQGRAHPMQT | 893 | 1.007 | 2.364 | 0.684 | 0.453 |
| GSGQLIASVVNQQT | 894 | 1.005 | 0.086 | 0.197 | 0.359 |
| GSSVRSLVGQNQQT | 895 | 1.004 | 3.840 | 0.412 | 0.608 |
| GGAGSAHSGQNQQT | 896 | 1.003 | 6.108 | 0.474 | 1.092 |
| GSDQNQQTMSSTRT | 897 | 1.003 | 2.428 | 1.306 | 0.835 |
| GSGQNQQMAGAFRT | 898 | 1.003 | 1.784 | 1.307 | 0.762 |
| GSLGNLQRGQNQQT | 899 | 1.003 | 0.895 | 0.947 | 0.385 |
| GSGPSISHGQNQQT | 900 | 1.000 | 0.000 | 0.614 | 0.665 |
| GSGQNQQT | 6406 | 1.000 | 1.000 | 1.000 | 1.000 |
| GSGQNQQSSFNVQT | 901 | 0.998 | 0.000 | 1.307 | 0.675 |
| GSGQNQQTGQATHN | 902 | 0.996 | 2.199 | 0.877 | 0.527 |

A second cross-species evolution screen was performed using an AAV capsid variant library with a modification in loop IV introduced as described above and passaging it once through NHPs (passage 1) and then subsequently injected it into two different strains of mice (passage 2), C57B1/6 and BALB/c. The fold-enrichment for each variant in the brain of each mouse species was calculated by systematic NGS enrichment analysis following RNA recovery and RT-PCR amplification. The fold enrichment values in the second passage in mice were compared to those fold enrichment values from the second pass that was performed in NHPs as described above. As shown in Table 22, when comparing the second pass fold enrichment values in the mice versus NHPs, 12 variants were identified that had a fold-enrichment value greater than 10 in all three animal groups. Further, 10 of these 12 variants comprised the SPH motif and a positive residue in one of the next three subsequent residues (Table 22).

TABLE 22
NGS fold-enrichment of AAV capsid variants
from a second passage (P2) in NHPs or
mice (C57BI/6 or BALB/c) following a
first passage in NHPs

| | SEQ | Fold | Fold | Fold |
|---|---|---|---|---|
| | ID | enrichment | enrichment | enrichment |
| Peptide | | over AAV9 in | over AAV9 in | over AAV9 in |
| Sequence | NO: | NHP P2 | BALB/c P2 | C57BI/6 P2 |
| VSGSPHSKAONOQT | 903 | 99.76 | 92.99 | 34.29 |
| CSGSPHSKAQNQQT | 904 | 85.1 | 66.74 | 22.19 |
| GSGSPHSKAQNOQT | 200 | 56.33 | 44.58 | 14.48 |
| GSGSPHRKAQNQQT | 905 | 46.39 | 42.47 | 14.11 |
| GRGSPHSKAQNOQT | 906 | 43.68 | 59.65 | 28.13 |
| GHDSPHKSGONOQT | 201 | 33.96 | 59.14 | 27.15 |
| GSGSPHSKAKNOQT | 208 | 31.27 | 41.51 | 14 |
| GSGSPHSKAQNKQT | 907 | 29.52 | 44.1 | 13.69 |
| GSGSPHSKAQTOQT | 908 | 24.27 | 41.75 | 18 |
| GODSPHKSGONOQT | 204 | 22.7 | 32.37 | 16.02 |
| GSGSTHASRONOOT | 909 | 11.04 | 23.71 | 10.67 |
| GHDSQHKSGONOQT | 404 | 10.36 | 21.3 | 13.55 |

Following the second passage in mice, a synthetic library was generated using those variants that demonstrated a fold-change in enrichment relative to wild-type AAV9 that was above 10 in the brain of either strain of mice, as measured by systematic NGS enrichment analysis following RNA recovery and RT-PCR amplification. There were approximately 500 variants in this synthetic library. This synthetic library was then injected back into both strains of mice (C57B1/6 and BALB/c; passage 3). RNA was recovered from the mouse brains, RT-PCR amplification was performed, and fold-enrichment relative to wild-type AAV9 was calculated by NGS analysis, which is provided in Table 23. As shown in Table 23, the variants with the greatest fold-enrichment in the brain in each strain, were highly correlated across strains (R2=0.8458).

TABLE 23
NGS fold-enrichment of AAV capsid variants in the brain from a third
passage (P3) inmice (C57BI/6 or BALB/c) following a first and
second passage in mice

| | SEQ | Fold enrichment | Fold enrichment | |
|---|---|---|---|---|
| | ID | over AAV9 in | over AAV9 in | |
| Peptide Sequence | NO: | BALB/c | C57BI/6 | Average |
| GSGSPHKYGQNQQT | 910 | 150.445 | 103.488 | 126.966 |
| GSGSPHKFGQNQQT | 911 | 73.364 | 60.304 | 66.834 |
| GHDSPHKSGQNQQT | 201 | 82.460 | 51.125 | 66.792 |
| GSGSPHSKAQNQQT | 200 | 60.312 | 65.853 | 63.083 |
| VSGSPHKFGQNQQT | 912 | 60.186 | 59.142 | 59.664 |
| GSGSPHSKAQNHQT | 913 | 63.486 | 51.647 | 57.566 |
| VSGSPHSKAQNQQT | 903 | 73.555 | 37.429 | 55.492 |
| GQDSPHKSGQNQQT | 204 | 63.898 | 43.752 | 53.825 |
| GSGSPHSKAQHQQT | 914 | 45.309 | 45.600 | 45.454 |
| GSGSPHKTYQNQQT | 915 | 50.283 | 35.460 | 42.871 |
| GSGSPHSKAQTQQT | 908 | 43.120 | 39.098 | 41.109 |
| VSGSPHASRQNQQT | 916 | 46.572 | 32.480 | 39.526 |
| GSGSPHSKAQNKQT | 907 | 39.848 | 35.596 | 37.722 |
| GSGSPHKFGKNQQT | 917 | 31.948 | 34.899 | 33.423 |
| GSGSPHASRQNQHT | 918 | 28.145 | 30.928 | 29.537 |
| GSHSPHKSGQNQQT | 919 | 22.948 | 35.412 | 29.180 |
| GSGQNQQRRMSPST | 920 | 4.576 | 53.520 | 29.048 |
| GSGSPHASRQNQQT | 205 | 28.866 | 29.139 | 29.003 |
| GSGSPHSKPQNQQT | 921 | 26.958 | 28.599 | 27.779 |
| GSGSPHKFGQKQQT | 922 | 39.597 | 14.927 | 27.262 |
| VSGSPHGARQNQQT | 923 | 30.985 | 22.634 | 26.810 |
| GSGSPHSKAQKQQT | 924 | 25.052 | 27.459 | 26.256 |
| GHSSPHRSGQNQQT | 212 | 16.982 | 35.081 | 26.032 |
| GSGSPHSKAKNQQT | 208 | 21.069 | 25.711 | 23.390 |
| GSHSPHKRGQNQQT | 925 | 24.054 | 20.262 | 22.158 |
| GRGSPHSKAQNQQT | 906 | 20.939 | 22.720 | 21.830 |
| GQSSPHRSGQNQQT | 229 | 9.916 | 26.608 | 18.262 |
| GSGQNRQRLKGLET | 926 | 3.937 | 31.022 | 17.480 |
| GSGSPHKLGQNQQT | 927 | 18.905 | 14.732 | 16.818 |
| GSGSPHKTSKNQQT | 928 | 14.654 | 17.606 | 16.130 |
| GSGSPHKIGQNQQT | 929 | 16.999 | 14.794 | 15.897 |
| GSGSPHKKNQNQQT | 209 | 25.633 | 5.605 | 15.619 |
| GSGSPHASRQNKQT | 206 | 10.738 | 20.347 | 15.542 |
| GSGSPHTRGQNQQT | 214 | 16.899 | 13.869 | 15.384 |
| GSGQDSPHVRNQQT | 930 | 15.340 | 14.646 | 14.993 |
| GSGSPHKTSQNQQT | 931 | 20.428 | 8.818 | 14.623 |
| GSGSPHASRKNQQT | 932 | 13.799 | 12.749 | 13.274 |
| GSGSPHASRQKQQT | 211 | 13.624 | 11.188 | 12.406 |
| GSHSPHKSGQKQQT | 933 | 6.700 | 17.736 | 12.218 |
| GSGSPHKTSQKQQT | 934 | 12.621 | 11.720 | 12.170 |
| GSGSPHVRGQNKQT | 935 | 13.174 | 11.017 | 12.095 |
| GSGSPHKTTQNQQT | 936 | 9.722 | 13.381 | 11.552 |
| CSGSPHSKAQNQQT | 904 | 11.772 | 9.447 | 10.610 |
| GSGPVRALRQNQQT | 937 | 3.369 | 17.431 | 10.400 |
| GSGSPHVRGQKQQT | 938 | 7.573 | 12.498 | 10.036 |
| GSGSPHRKAQNQQT | 905 | 12.308 | 7.349 | 9.828 |
| GRGSPHASRQNQQT | 318 | 11.903 | 6.780 | 9.342 |
| CSGSPHKTSQNQQT | 939 | 11.167 | 6.631 | 8.899 |
| CSHSPHKSGQNQQT | 940 | 11.356 | 6.304 | 8.830 |
| GSGSPHSKDQNQQT | 604 | 3.492 | 10.236 | 6.864 |

Taken together, these results demonstrate that after 3 rounds of screening of this AAV9 variant library with loop IV modifications in NHP and mice, many AAV capsid variants outperformed the wild-type AAV9, for example, in penetration of the blood brain barrier (BBB) and spinal cord expression. These capsid variants were able to cross-species, evidenced by expression and tropism in the NHP brain/spinal cord as well as in the brain of two different mouse species.

### Example 3. Individual Capsid Characterization in Mice

The goal of these experiments was to determine the transduction level, tropism, ability to cross the blood brain barrier, and overall spatial distribution in the central nervous system (CNS) of 2 capsid variants selected from the study described in Example 2 relative to AAV9 following intravenous injection in mice. The 2 capsid variants were TTM-001 (SEQ ID NO: 981 (amino acid) and 983 (DNA), comprising SEQ ID NO: 941) and TTM-002 (SEQ ID NO: 982 (amino acid) and 984 (DNA), comprising SEQ ID NO: 2), as outlined in Table 3 above. The amino acid and DNA sequences of TTM-001 and TTM-002 are provided, e.g., in Tables 4 and 5, respectively.

AAV particles were generated with each of these capsid variants encapsulating a luciferase-EGFP transgene driven by a CMV/chicken beta actin promoter in a single stranded viral genome. Each capsid variant and AAV9 control were tested by intravenously administering by tail vein injection, the AAV particle formulation at 5e11 VG/dose (2.5E13 vg/kg) to three female BALB/c mice. The in-life period was 28 days and then various CNS and peripheral tissues were collected for measuring transgene mRNA, transgene protein, and viral DNA (biodistribution).

At 28 days post-injection of the AAV particles encapsulated in the TTM-001 capsid variant (AAV_TTM-001), mice were injected with luciferin and their brains were harvested for IVIS imaging. Robust luciferase signal was observed in mice injected with AAV particles encapsulated in the TTM-001 capsid variant, and this was greatly increased relative to AAV particles encapsulated in the wild-type AAV9 control capsid.

The brains isolated from mice injected with the AAV particles encapsulated in the TTM-001 capsid variant (AAV_TTM-001) or the TTM-002 capsid variant (AAV_TTM-002) were assayed by qPCR for the presence of transgene RNA as a measure of transgene expression, and the presence of viral DNA as a measure of viral genome levels. Data were provided as fold over AAV9 (Table 24). As shown in Table 24, when compared to the wild-type AAV9 capsid control, TTM-001 and TTM-002 demonstrated a 30-fold and 66-fold increase, respectively, in transgene mRNA levels and expression in the brain, indicative of enhanced payload delivery. This correlated with a 32-fold (TTM-001) and 47-fold (TTM-002) increase, respectively, in viral genome (DNA) concentrations in the brain relative to the AAV9 capsid control, which is indicative of enhanced CNS tropism and transduction (Table 24).

TABLE 24
Transgene mRNA and viral genome levels (DNA) in mice relative to the AAV9 control

| Measure | Tissue | AAV9 | TTM-001 | TTM-002 |
|---|---|---|---|---|
| mRNA (transgene expression) | Brain | 1.0 | 30.4503 | 66.2161 |
| DNA (viral genome quantification) | Brain | 1.0 | 32.0315 | 47.2810 |
| mRNA (transgene expression) | Liver | 1.0 | 1.2356 | 0.2016 |
| DNA (viral genome quantification) | Liver | 1.0 | 0.4802 | 0.0277 |

The brain tissues and spinal cords of the mice were also subjected to anti-GFP immunohistochemistry staining to evaluate overall CNS tropism and biodistribution. Immunohistochemical staining correlated with the qPCR analysis, as TTM-001 and TTM-002 showed significantly stronger staining and payload expression in the brain and spinal cord, as compared to the AAV9 control. More specifically, TTM-001 and TTM-02 demonstrated localization and strong payload expression and transduction in the mid-brain region, with increased staining observed in the hippocampus and thalamus, as well as in the brain stem, compared to AAV9. Less staining was observed in the cortical regions of the brain compared to the midbrain. However, staining in these cortical regions was stronger for TTM-001 and TTM-002 compared to the AAV9 control. It also appeared that the TTM-001 and TTM-002 capsid variants were able to transduce non-neuronal cells, including glial cells and oligodendrocytes. With respect to the spinal cord, staining and payload expression for TTM-01 and TTM-002 were localized to the ventral horns of the grey matter.

Peripheral tissues were also isolated from the mice intravenously injected with the AAV particles encapsulated in the TTM-001 capsid variant or the TTM-002 capsid variant for analysis by qPCR and/or GFP immunohistochemical staining. Transgene mRNA levels and viral genome DNA levels were quantified in the liver by qPCR and the fold over AAV9 was calculated for each capsid variant (Table 24). TTM-001 resulted in similar levels of payload expression (mRNA levels) as compared to wild-type AAV9, but only half as much viral genome DNA was quantified in the liver compared to AAV9. TTM-002 demonstrated greatly reduced mRNA and viral genome DNA levels in the liver compared to AAV9. GFP immunohistochemical staining of the spleen, heart, skeletal muscle, kidneys, and lungs of mice injected with AAV particles encapsulated in the TTM-001 capsid variant or the TTM-002 capsid variant showed similar levels of payload expression as compared to those mice injected with AAV particles encapsulated in the wild-type AAV9 control capsid.

Taken together, these data demonstrate that TTM-001 and TTM-002 are enhanced CNS tropic capsids in mice that can infect non-neuronal cells. Additionally, these capsid variants were able to successfully penetrate the blood brain barrier following intravenous injection.

### Example 4. Maturation of TTM-001 and TTM-002 Capsid in NHPs

This Example describes maturation of the AAV9 capsid variants, TTM-001 (SEQ ID NO: 981 (amino acid) and 983 (DNA), comprising SEQ ID NO: 941) and TTM-002 (SEQ ID NO: 982 (amino acid) and 984 (DNA), comprising SEQ ID NO: 2) in NHPs to further enhance their transduction and biodistribution in the central nervous system as well as other tissues, and evolve the AAV capsid variants to provide further cross-species compatibility. Two approaches were used to mature the TTM-001 and TTM-002 capsid sequences in order to randomize and mutate within and around the peptide insert comprised within loop IV of the capsid variant. As many of the AAV capsid variants that demonstrated the greatest fold-enrichment in the NHP brain relative wild-type AAV9 comprised an SPH motif in the same position (e.g., immediately subsequent to position 455, relative to a reference sequence numbered according to the amino acid sequence of SEQ ID NO: 138) (see Example 2), the SPH motif was not mutated in either approach to mature the TTM-001 and TTM-002 capsid variants. In the first maturation approach, sets of three contiguous amino acids were randomized across the mutagenesis region in the TTM-001 and TTM-002 sequences, which spanned from position 450 to position 466, numbered according to SEQ ID NO: 981 and 982. In the second maturation approach, mutagenic primers were used to introduce point mutations at a low frequency, scattered across the mutagenesis region in the TTM-001 and TTM-002 sequences ranging from position 449 to position 466, numbered according to SEQ ID NO: 981 and 982. AAV capsid variants arising from each maturation approach for TTM-001 and TTM-002 were pooled together, for subsequent testing and characterization in NHPs.

The library of pooled matured AAV capsid variants generated using the first maturation approach and the second maturation approach for the TTM-001 and TTM-002 AAV capsid variants were injected into two NHPs. After a period in life, the brains, heart, liver, muscle, and DRG of the NHPs were isolated and RNA was extracted. Following RNA recovery and RT-PCR amplification, a systematic NGS enrichment analysis was performed to calculate the fold enrichment ratio relative to an AAV9 control, and the peptides comprised within the variants were identified.

Following the RNA recovery and NGS analysis from the second maturation approach, approximately 680,000 capsid variants were identified. The 680,000 matured capsid variants were then filtered based on samples with a raw virus count greater than 10 and a coefficient of variance (CV) of less than 1, which was calculated for each peptide across the brain samples taken from the two NHPs. Those that had a CV value <1 were identified, as these were the peptides that were reliably detected in the majority of samples isolated from the brains of the two NHPs. Using this filtering criteria, this led to approximately 64,000 matured capsid variants.

Table 25 provides the peptide sequences of the matured capsid variants having a raw virus count greater than 10, a CV of less than 1 for the brain samples isolated, and that also demonstrated a 50-fold or greater fold-increase in expression in the brain relative to the AAV9 control in both mice and NHPs. The matured variants in Table 25, were also those variants that had a fold-change in expression that was less than 2 relative to the AAV9 control in the liver and the DRG. Applying these criteria, approximately 350 matured capsid variants were identified that demonstrated high transduction in the brain in NHPs and mice, cross-species compatibility in mice and NHPs, and were de-targeted in the liver and DRG, relative to the AAV9 control. Several variants as shown in Table 25, led to greater than 10⁰-fold increase in expression relative to AAV9 in the NHP and/or mouse brain, with one variant resulting in a greater than 200-fold increase in expression relative to AAV9 in both species.

Fold-change in expression for the TTM-001 and TTM-002 matured variants in Table 25 that showed increased expression in the brain of the NHPs and mice, were also calculated for the DRG, muscle, liver (RNA and DNA), and heart of the NHPs following each maturation approach. As shown in Table 25, many variants were de-targeted in the peripheral tissues with a lower fold-change in expression relative to the AAV9 control, demonstrating CNS-specific tropism and a preferential transduction of the brain and CNS. Some variants demonstrated increased expression to AAV9 in multiple tissues, including the brain and peripheral tissues, demonstrating pan-tropism.

TABLE 25
NGS fold-enrichment of TTM-001 and TTM-002 matured AAV capsid variants in the
brain of NHPs and mice

| | | Fold Enrichment relative to AAV9 | | | | | | |
|---|---|---|---|---|---|---|---|---|
| | SEQ | | | | | Liver | Liver | |
| | ID | Brain | DRG | Heart | Muscle | RNA | DNA | Brain |
| Sequence | NO: | (NHP) | (NHP) | (NHP) | (NHP) | (NHP) | (NHP) | (Mouse) |
| KTIIGSGSPHSKAQNRHT | 3239 | 217.176 | 0.000 | 0.000 | 0.000 | 0.000 | 0.000 | 210.515 |
| KTFPGSGSPHSKVQNQQT | 3240 | 199.720 | 0.000 | 0.000 | 0.000 | 0.000 | 0.967 | 97.703 |
| KTEKMSGSPHSKAQNQQT | 3241 | 169.461 | 0.523 | 0.000 | 0.000 | 0.000 | 0.158 | 109.161 |
| KEINGRGSPHSKAQNQQT | 3527 | 134.390 | 0.239 | 0.000 | 0.000 | 0.000 | 0.232 | 52.311 |
| KTVNRNGSPHSKAQNQQT | 3528 | 133.016 | 0.000 | 0.416 | 0.000 | 0.000 | 0.000 | 85.361 |
| KTVNGSGSPHSKARDQQT | 3242 | 124.789 | 0.123 | 0.039 | 0.312 | 0.569 | 0.454 | 132.137 |
| KTFNGSGSPHSKAPNLQT | 3243 | 121.436 | 0.000 | 0.167 | 0.000 | 0.000 | 0.015 | 168.920 |
| KTEKTSGSPHSKAQNQQT | 3244 | 120.337 | 0.000 | 0.355 | 0.000 | 0.000 | 0.119 | 101.467 |
| KTINGSGSPHSKAHVRQT | 3245 | 119.798 | 0.000 | 0.000 | 0.262 | 0.694 | 1.039 | 165.590 |
| KTVNGSGSPHSKAPNQHT | 3246 | 117.207 | 0.000 | 0.109 | 0.000 | 0.000 | 0.074 | 51.008 |
| KTEKISGSPHSKAQNQQT | 3247 | 116.603 | 0.000 | 0.000 | 0.000 | 0.000 | 0.426 | 102.978 |
| KTINGPGSPHSKAHNQQT | 3529 | 115.742 | 0.146 | 0.000 | 0.235 | 0.000 | 0.513 | 52.508 |
| KTVNGSGSPHSKTQSQQT | 3248 | 115.086 | 0.000 | 0.726 | 0.000 | 0.000 | 0.340 | 63.248 |
| TTINGSGSPHSKAQNQQT | 3249 | 114.856 | 1.340 | 14.856 | 0.827 | 1.281 | 0.957 | 72.058 |
| KSINESGSPHSKAQNQQI | 3250 | 113.833 | 0.000 | 0.000 | 0.000 | 0.000 | 0.000 | 67.649 |
| KTERTSGSPHSKAQNQQT | 3251 | 112.957 | 0.000 | 0.009 | 0.000 | 1.128 | 0.207 | 117.374 |
| KTINGSGSPHSKAQPAKT | 3252 | 111.472 | 0.331 | 0.000 | 1.089 | 0.044 | 1.796 | 215.275 |
| KTEKSSGSPHSKAQNQQT | 3253 | 107.470 | 0.000 | 0.016 | 0.014 | 0.977 | 0.179 | 100.177 |
| KTSYGNGSPHSKAQNQQT | 3530 | 105.937 | 0.000 | 0.000 | 0.000 | 0.000 | 0.114 | 105.894 |
| KTEKGSGSPHSKAQNQQT | 3254 | 105.614 | 0.053 | 0.031 | 0.000 | 0.586 | 0.169 | 84.653 |
| KTINGSGSPHSKSQTQQN | 3255 | 104.474 | 0.000 | 0.131 | 0.000 | 0.084 | 0.038 | 54.021 |
| KTERISGSPHSKAQNQQT | 3256 | 103.692 | 0.000 | 0.000 | 0.000 | 0.062 | 0.370 | 89.637 |
| KTERASGSPHSKAQNQQT | 3257 | 103.669 | 0.000 | 0.000 | 0.000 | 0.127 | 0.070 | 115.550 |
| KELHGSGSPHSKAQNQQT | 3258 | 102.680 | 0.000 | 0.000 | 0.000 | 1.634 | 0.592 | 96.554 |
| KAINGSGSPHSKAQNLAT | 3259 | 101.954 | 0.000 | 10.954 | 8.655 | 0.298 | 0.239 | 116.685 |
| KTVNGSGSPHSKSQNQLT | 3260 | 101.327 | 0.000 | 0.035 | 0.000 | 0.000 | 0.025 | 80.716 |
| KTERNSGSPHSKAQNQQT | 3261 | 99.892 | 0.000 | 0.000 | 0.000 | 0.000 | 0.107 | 87.392 |
| KSVNGNGSPHSKAQNQQT | 3531 | 99.385 | 0.000 | 1.329 | 0.000 | 0.359 | 0.079 | 51.016 |
| KTFNGSGSPHSKAQGQQT | 3262 | 99.253 | 0.000 | 0.208 | 0.000 | 0.128 | 0.099 | 81.459 |
| KTINGSGSPHGWVQNQQT | 3532 | 97.122 | 0.000 | 0.000 | 0.000 | 1.240 | 1.975 | 290.720 |
| KTERVSGSPHSKAQNQQT | 3263 | 96.943 | 0.000 | 0.000 | 0.000 | 0.000 | 0.144 | 135.438 |
| KTINGSGSPHSKALNRQS | 3264 | 96.843 | 0.136 | 0.532 | 0.000 | 0.042 | 0.178 | 55.945 |
| KTERLSGSPHSKAQNQQT | 3265 | 95.857 | 0.000 | 0.004 | 0.005 | 0.126 | 0.260 | 102.372 |
| KTDNGSGSPHSKAHNQQT | 3266 | 95.164 | 0.000 | 0.000 | 0.000 | 0.000 | 0.027 | 55.313 |
| KTFHGSGSPHSKTQNQQT | 3267 | 94.714 | 0.000 | 0.210 | 0.120 | 0.000 | 0.000 | 51.119 |
| KTINGGGSPHSKAQTQQI | 3533 | 92.345 | 0.000 | 0.000 | 0.000 | 0.000 | 0.023 | 54.199 |
| KTSNGSGSPHSKAQNPPT | 3268 | 91.528 | 0.000 | 0.000 | 0.000 | 0.000 | 0.039 | 51.541 |
| ETINGSGSPHSKAQNLQT | 3269 | 90.969 | 0.221 | 1.023 | 0.197 | 0.179 | 0.813 | 107.216 |
| KTVHGNGSPHSKAQNQQT | 3534 | 90.073 | 0.000 | 0.000 | 0.000 | 0.000 | 0.304 | 97.003 |
| NTINGSGSPHSKAQNQQT | 3270 | 90.017 | 1.712 | 1.261 | 1.171 | 0.923 | 0.540 | 55.179 |
| KTINGGGSPHSKAQNQQC | 3535 | 89.301 | 0.219 | 0.000 | 0.000 | 0.287 | 0.319 | 53.840 |
| KTENMSGSPHSKAQNQQT | 3271 | 89.247 | 0.000 | 0.000 | 0.000 | 0.000 | 0.260 | 130.568 |
| KTENVSGSPHSKAQNQQT | 3272 | 88.506 | 0.000 | 0.000 | 0.000 | 0.964 | 0.112 | 108.591 |
| KTSSGSGSPHSKAQYQQT | 3273 | 87.304 | 0.000 | 0.000 | 0.000 | 0.000 | 0.299 | 58.143 |
| KTIDGGGSPHSKAQNKQT | 3536 | 85.019 | 0.000 | 0.000 | 0.000 | 0.000 | 0.477 | 55.517 |
| KTEKVSGSPHSKAQNQQT | 3274 | 84.558 | 0.000 | 0.022 | 0.000 | 0.873 | 0.424 | 112.185 |
| KAINGSGSPHSKAQDQET | 3275 | 84.080 | 0.000 | 0.000 | 0.000 | 0.194 | 0.027 | 87.637 |
| KTCNKSGSPHSKAQNQQT | 3276 | 83.992 | 0.000 | 0.000 | 0.165 | 0.283 | 0.000 | 119.496 |
| KTINGGGSPHSKAQNQLI | 3537 | 83.881 | 0.000 | 0.000 | 0.000 | 0.046 | 0.387 | 78.383 |
| KNINGGGSPHSKAQNQQT | 3538 | 83.083 | 0.000 | 0.042 | 0.000 | 0.000 | 0.000 | 75.913 |
| KTEHLSGSPHSKAQNQQT | 3277 | 83.080 | 0.000 | 0.000 | 0.012 | 0.021 | 0.189 | 69.494 |
| KAEMGSGSPHSKAQNQQT | 3278 | 83.049 | 0.000 | 0.020 | 0.000 | 0.768 | 0.112 | 135.019 |
| KATNGSGSPHSKAQNHQT | 3279 | 82.627 | 0.000 | 0.176 | 0.000 | 0.155 | 0.057 | 66.207 |
| KAIKGSGSPHSKAQDQQT | 3280 | 82.258 | 0.000 | 0.000 | 0.000 | 0.108 | 0.000 | 85.178 |
| KTINGGGSPHSKSQNQLT | 3539 | 82.231 | 0.000 | 0.070 | 0.000 | 0.000 | 0.498 | 126.986 |
| KTVNGNGSPHSKAQNKQT | 3540 | 81.481 | 0.000 | 0.000 | 0.000 | 0.000 | 0.122 | 69.455 |
| KTINGSGSPHSKGHWQQT | 3281 | 81.434 | 0.000 | 0.000 | 0.000 | 0.000 | 1.011 | 65.252 |
| KTDKTSGSPHSKAQNQQT | 3282 | 81.430 | 0.000 | 0.000 | 0.000 | 1.362 | 0.291 | 169.515 |
| KTFKGSGSPHSKAPNQQT | 3283 | 80.890 | 0.000 | 0.000 | 0.000 | 0.000 | 0.017 | 71.144 |
| KTVNGSGSPHSKAQNQLI | 3284 | 80.509 | 0.000 | 0.000 | 0.000 | 0.000 | 0.166 | 71.156 |
| KTINGSGSPHSKRPEQQT | 3285 | 80.418 | 0.000 | 0.013 | 0.000 | 0.149 | 0.361 | 50.319 |
| KTINGSGSPHSKAQRTMT | 3286 | 80.388 | 0.000 | 0.022 | 0.170 | 1.812 | 1.025 | 100.248 |
| KTEKASGSPHSKAQNQQT | 3287 | 80.285 | 0.000 | 0.041 | 0.000 | 0.000 | 0.261 | 90.390 |
| KSDQGSGSPHSKAQNQQT | 3288 | 80.076 | 0.000 | 0.000 | 0.000 | 0.993 | 0.124 | 151.911 |
| KTEITSGSPHSKAQNQQT | 3289 | 79.620 | 0.000 | 0.163 | 0.000 | 0.332 | 0.074 | 76.686 |
| KTDKSSGSPHSKAQNQQT | 3290 | 79.470 | 0.055 | 0.012 | 0.000 | 1.437 | 0.367 | 141.351 |
| KTIDGSGSPHSKAQNQQH | 3291 | 79.090 | 0.000 | 0.000 | 0.000 | 0.136 | 0.049 | 57.914 |
| KTVNGNGSPHSKAQNQHT | 3541 | 78.849 | 0.000 | 0.000 | 0.000 | 0.000 | 0.045 | 54.086 |
| KNINGSGSPHSKAQNQQT | 3292 | 78.445 | 0.000 | 0.000 | 0.000 | 0.571 | 0.177 | 89.719 |
| KTETHSGSPHSKAQNQQT | 3293 | 77.974 | 0.000 | 0.067 | 0.000 | 0.000 | 0.512 | 57.287 |
| KTINGGGSPHSKALNQQN | 3542 | 77.822 | 0.000 | 0.131 | 0.000 | 0.000 | 0.274 | 69.884 |
| KTINGSGSPHSKALHQHT | 3294 | 77.502 | 0.000 | 0.052 | 0.041 | 0.000 | 0.188 | 68.196 |
| KTINGTGSPHSKAQNHQI | 3543 | 77.089 | 0.171 | 0.000 | 0.000 | 0.000 | 0.166 | 54.281 |
| KTINGSGSPHSKAQHRIT | 3295 | 76.849 | 0.105 | 0.499 | 0.170 | 1.424 | 0.214 | 127.000 |
| KTINGSGSPHSKAQYIHT | 3296 | 76.170 | 0.000 | 0.014 | 0.033 | 1.523 | 0.168 | 59.649 |
| KTENISGSPHSKAQNQQT | 3297 | 76.072 | 0.000 | 0.000 | 0.000 | 0.115 | 0.132 | 83.118 |
| KTIIGGGSPHSKAHNQQT | 3544 | 75.872 | 0.000 | 0.050 | 0.000 | 0.000 | 0.235 | 65.492 |
| KTINGSGSPHSKAQKFET | 3298 | 75.788 | 0.000 | 0.000 | 0.028 | 0.108 | 0.093 | 65.588 |
| KTSNESGSPHSKAQNHQT | 3299 | 75.720 | 0.000 | 0.000 | 0.000 | 0.169 | 0.217 | 70.590 |
| KTINGSGSPHSKAQFPST | 3300 | 75.677 | 0.000 | 0.004 | 0.000 | 0.849 | 0.127 | 119.712 |
| KTERPSGSPHSKAQNQQT | 3301 | 75.669 | 0.000 | 0.029 | 0.000 | 0.000 | 0.156 | 73.894 |
| KTINGNGSPHSKAQNPLT | 3545 | 75.269 | 0.000 | 0.000 | 0.000 | 0.366 | 0.000 | 53.583 |
| KSIKGNGSPHSKAQNQQT | 3546 | 75.196 | 0.000 | 0.000 | 0.000 | 0.000 | 0.000 | 90.251 |
| KTERMSGSPHSKAQNQQT | 3302 | 74.910 | 0.000 | 0.000 | 0.000 | 0.100 | 0.151 | 122.812 |
| KTERSSGSPHSKAQNQQT | 3303 | 74.853 | 0.000 | 0.071 | 0.000 | 1.036 | 0.056 | 125.538 |
| KTELHSGSPHSKAQNQQT | 3304 | 74.620 | 0.000 | 0.000 | 0.000 | 0.021 | 0.089 | 53.124 |
| KTELTSGSPHSKAQNQQT | 3305 | 74.548 | 0.000 | 0.000 | 0.000 | 0.537 | 0.421 | 100.311 |
| KTINGSGSPHSKAHNQQR | 3306 | 74.272 | 0.562 | 0.486 | 0.047 | 0.956 | 0.057 | 107.301 |
| KTINGGGSPHSKAQSQQI | 3547 | 74.264 | 0.000 | 0.000 | 0.000 | 0.000 | 0.235 | 67.651 |
| KTINGSGSPHSKAQAIKT | 3307 | 74.261 | 0.255 | 0.000 | 0.000 | 0.186 | 0.132 | 73.560 |
| KTENTSGSPHSKAQNQQT | 3308 | 74.061 | 0.000 | 0.000 | 0.218 | 0.233 | 0.730 | 96.249 |
| KTIDGSGSPHSKGQNRQT | 3309 | 73.930 | 0.000 | 0.000 | 0.000 | 0.106 | 0.091 | 63.626 |
| KNINGSGSPHSKAQSQQT | 3310 | 73.757 | 0.000 | 0.000 | 0.000 | 0.000 | 0.041 | 57.432 |
| KTINGSVSPHGKAQNQLT | 3548 | 73.525 | 0.000 | 0.061 | 0.067 | 0.000 | 0.053 | 51.358 |
| KTSNASGSPHSKAQNQLT | 3311 | 73.501 | 0.000 | 0.000 | 0.297 | 0.000 | 0.313 | 150.401 |
| KTEARSGSPHSKAQNQQT | 3312 | 73.349 | 0.000 | 0.000 | 0.000 | 0.695 | 0.118 | 62.903 |
| KTEKNSGSPHSKAQNQQT | 3313 | 73.347 | 0.000 | 0.000 | 0.044 | 0.159 | 0.021 | 74.393 |
| KTANGSGSPHSKAQYQQT | 3314 | 73.038 | 0.000 | 0.000 | 0.000 | 0.153 | 0.160 | 139.451 |
| KTVNGSGSPHSKAQYQHT | 3315 | 72.847 | 0.000 | 0.000 | 0.000 | 0.000 | 0.130 | 54.158 |
| KTINGSGSPHTKAQNPQS | 3316 | 72.594 | 0.000 | 0.000 | 0.000 | 0.000 | 0.130 | 62.508 |
| KTINGSGSPHSKGQNPPT | 3317 | 72.339 | 0.000 | 0.206 | 0.000 | 0.000 | 0.041 | 134.808 |
| KTIIGSGSPHSKAQHQLT | 3318 | 72.291 | 0.000 | 0.000 | 0.000 | 0.000 | 0.000 | 100.144 |
| KTINGSGSPHSKAQSPPT | 3319 | 71.632 | 0.069 | 0.047 | 0.274 | 0.179 | 0.425 | 97.111 |
| NTIYGSGSPHSKAQNQQT | 3320 | 71.267 | 1.739 | 0.000 | 273.69 | 0.000 | 0.209 | 59.707 |
| KTINGSGSPHSKAQAKLT | 3321 | 71.154 | 0.000 | 0.273 | 0.017 | 1.591 | 0.777 | 130.132 |
| KTDKNSGSPHSKAQNQQT | 3322 | 70.964 | 0.000 | 0.000 | 0.000 | 0.070 | 0.123 | 62.932 |
| KTINGSGSPHSKTKSQQT | 3323 | 70.891 | 0.000 | 0.568 | 0.045 | 0.418 | 0.496 | 83.923 |
| KTINGSGSPHSKAQDRPT | 3324 | 70.831 | 0.132 | 0.006 | 0.000 | 0.039 | 0.379 | 66.800 |
| KTINGIGSPHSKAQNLGT | 3549 | 70.543 | 0.000 | 0.071 | 0.000 | 0.000 | 0.135 | 104.769 |
| KTINGSGSPHSKAQSQQL | 3325 | 70.539 | 0.000 | 0.000 | 0.000 | 0.000 | 0.041 | 51.126 |
| KTENLSGSPHSKAQNQQT | 3326 | 70.303 | 0.070 | 0.000 | 0.000 | 0.395 | 0.470 | 107.385 |
| KTINGSGSPHSKAQAFHT | 3327 | 70.159 | 0.033 | 0.000 | 0.058 | 0.762 | 0.119 | 86.268 |
| KTINGSGSPHSKAQKQQD | 3328 | 70.116 | 0.000 | 0.024 | 0.000 | 0.064 | 0.083 | 110.196 |
| KTFSGSGSPHSKAQNLQT | 3329 | 70.035 | 0.000 | 0.327 | 0.303 | 0.000 | 0.228 | 70.917 |
| KAINGSGSPHSKAQNAQT | 3330 | 69.651 | 0.000 | 0.000 | 0.000 | 0.023 | 0.142 | 72.160 |
| KTESWSGSPHSKAQNQQT | 3331 | 69.144 | 0.000 | 0.000 | 0.000 | 0.000 | 0.019 | 67.699 |
| KTTNGSGSPHSKAHNQLT | 3332 | 69.062 | 0.000 | 0.000 | 0.000 | 0.708 | 0.000 | 65.505 |
| KTVNGNGSPHSKAQNHQT | 3550 | 68.889 | 0.000 | 0.000 | 0.000 | 0.000 | 0.030 | 52.482 |
| KTEDKSGSPHSKAQNQQT | 3333 | 68.813 | 0.000 | 0.000 | 0.000 | 0.000 | 0.000 | 70.071 |
| KTESASGSPHSKAQNQQT | 3334 | 68.651 | 0.000 | 0.000 | 0.000 | 0.274 | 0.084 | 80.500 |
| KTNNGSGSPHSKAQNQQY | 3335 | 68.530 | 0.000 | 0.040 | 0.000 | 0.000 | 0.059 | 82.656 |
| KTSNGGGSPHSKAQNLQT | 3551 | 68.311 | 0.000 | 0.052 | 0.000 | 0.000 | 0.000 | 124.871 |
| KTDKMSGSPHSKAQNQQT | 3336 | 68.167 | 0.000 | 0.000 | 0.000 | 0.017 | 0.205 | 88.234 |
| KEVHGSGSPHSKAQNQQT | 3337 | 67.901 | 0.000 | 0.000 | 0.000 | 0.727 | 0.000 | 100.111 |
| KTINGSGSPHSKAQKLNT | 3338 | 67.782 | 0.073 | 0.092 | 0.000 | 1.232 | 0.201 | 68.637 |
| KTINGGGSPHSKSQNQHT | 3552 | 67.773 | 0.000 | 0.057 | 0.000 | 0.000 | 0.220 | 100.748 |
| KTVNGGGSPHSKAQSQQT | 3553 | 67.634 | 0.000 | 0.055 | 0.000 | 0.000 | 0.210 | 160.711 |
| KTTNGSGSPHSKAQYQHT | 3339 | 67.325 | 0.000 | 0.000 | 0.000 | 1.378 | 0.080 | 83.337 |
| KTISGSGSPHSKAQYQHT | 3340 | 66.739 | 0.000 | 0.000 | 0.000 | 0.000 | 0.191 | 59.822 |
| KTESTSGSPHSKAQNQQT | 3341 | 66.649 | 0.000 | 0.009 | 0.000 | 1.688 | 0.176 | 95.861 |
| KTINGSGSPHSKSQNVQT | 3342 | 66.627 | 0.000 | 0.190 | 0.000 | 0.202 | 0.188 | 56.672 |
| KSINGSGSPHSKAQAQQT | 3343 | 66.464 | 0.000 | 0.711 | 0.000 | 0.148 | 0.111 | 78.451 |
| KTVNGSGSPHSKAQNLQA | 3344 | 66.379 | 0.000 | 0.000 | 0.000 | 0.000 | 0.132 | 50.934 |
| KTVRDSGSPHSKAQNQQT | 3345 | 66.056 | 0.000 | 0.025 | 0.000 | 0.129 | 0.461 | 142.600 |
| KTFNASGSPHSKAPNQQT | 3346 | 65.392 | 0.208 | 0.000 | 0.000 | 0.215 | 0.156 | 66.275 |
| KTDRMSGSPHSKAQNQQT | 3347 | 65.143 | 0.000 | 0.000 | 0.000 | 0.332 | 0.103 | 104.890 |
| KTINGSGSPHSKAQTPPT | 3348 | 64.657 | 0.010 | 0.015 | 0.014 | 0.200 | 0.207 | 54.179 |
| ETIKGSGSPHSKAQNQQT | 3349 | 64.609 | 0.000 | 0.000 | 0.144 | 0.000 | 0.024 | 67.201 |
| KNHIGSGSPHSKAQNQQT | 3350 | 64.535 | 0.000 | 0.000 | 0.000 | 1.253 | 0.187 | 70.356 |
| KTINGSGSPHSKAQYQHA | 3351 | 64.435 | 0.000 | 0.000 | 0.024 | 0.993 | 0.097 | 57.278 |
| KTIPIDGSPHSKAQNQQT | 3554 | 64.421 | 0.000 | 0.047 | 0.000 | 0.234 | 0.936 | 76.826 |
| KTINGSGSPHSKAQGQQA | 3352 | 64.128 | 0.000 | 0.185 | 0.000 | 0.063 | 0.195 | 64.116 |
| KTFNGSGSPHNKAQNHQT | 3353 | 64.060 | 0.000 | 0.000 | 0.035 | 0.094 | 0.317 | 67.757 |
| KESDGSGSPHSKAQNQQT | 3354 | 63.766 | 0.000 | 0.000 | 0.000 | 0.567 | 0.146 | 115.231 |
| KTINGSGSPHSKAQPPAT | 3355 | 63.510 | 0.048 | 0.030 | 0.031 | 0.126 | 0.302 | 117.453 |
| KTINGSGSPHSKAQERPT | 3356 | 63.460 | 0.000 | 0.011 | 0.000 | 0.810 | 0.173 | 57.506 |
| KTIKGSGSPHSKAQDLQT | 3357 | 63.260 | 0.000 | 0.000 | 0.000 | 0.000 | 0.218 | 58.576 |
| KTDLKSGSPHSKAQNQQT | 3358 | 63.152 | 0.000 | 0.000 | 0.012 | 0.285 | 0.377 | 62.687 |
| KTINGGGSPHSKAQNPPT | 3555 | 63.041 | 0.000 | 0.082 | 0.000 | 0.000 | 0.057 | 64.045 |
| KTINGSGSPHSKAQAMHT | 3359 | 62.756 | 0.000 | 0.000 | 0.010 | 0.976 | 0.393 | 84.056 |
| KTVPNSGSPHSKAQNQQT | 3360 | 62.540 | 0.000 | 0.000 | 0.011 | 0.202 | 0.161 | 93.793 |
| KTVIGSGSPHSKALNQQT | 3361 | 62.358 | 0.000 | 0.310 | 0.000 | 0.062 | 0.245 | 60.369 |
| KTINGSGSPHSKAQHPST | 3362 | 62.255 | 0.000 | 0.044 | 0.000 | 1.345 | 0.301 | 101.103 |
| KTINGLGSPHSKSQNQQT | 3556 | 62.170 | 0.000 | 0.157 | 0.000 | 0.146 | 0.107 | 64.139 |
| KTINGTGSPHSKAQNQQM | 3557 | 62.151 | 0.000 | 0.000 | 0.000 | 0.000 | 0.000 | 62.376 |
| KTINGSGSPHSKAPGLQT | 3363 | 62.043 | 0.007 | 0.000 | 0.005 | 0.651 | 0.210 | 144.610 |
| KTINGSGSPHSKAQGIRT | 3364 | 61.952 | 0.041 | 0.000 | 0.012 | 0.897 | 0.502 | 155.013 |
| KTESHSGSPHSKAQNQQT | 3365 | 61.947 | 0.000 | 0.000 | 0.000 | 1.480 | 0.106 | 52.506 |
| KTINGSGSPHSKAQAPAT | 3366 | 61.934 | 0.000 | 0.169 | 0.015 | 0.696 | 0.197 | 127.420 |
| KTINGSGSPHSKSQSQQI | 3367 | 61.870 | 0.000 | 0.000 | 0.000 | 0.200 | 0.175 | 64.027 |
| KAEHGSGSPHSKAQNQQT | 3368 | 61.830 | 0.000 | 0.000 | 0.000 | 0.772 | 0.184 | 116.201 |
| KTEDRSGSPHSKAQNQQT | 3369 | 61.756 | 0.000 | 0.000 | 0.000 | 1.004 | 0.408 | 66.887 |
| KNCLGSGSPHSKAQNQQT | 3370 | 61.442 | 0.000 | 0.036 | 0.000 | 1.849 | 0.026 | 82.488 |
| KTDRGSGSPHSKAQNQQT | 3371 | 61.419 | 0.000 | 0.004 | 0.000 | 0.211 | 0.316 | 74.256 |
| KTINGSGSPHSKAQIPPT | 3372 | 61.258 | 0.000 | 0.000 | 0.000 | 0.758 | 0.115 | 87.661 |
| KTVKGSGSPHSKAQDQQT | 3373 | 61.175 | 0.000 | 0.041 | 0.000 | 0.432 | 0.090 | 58.114 |
| KNADGSGSPHSKAQNQQT | 3374 | 60.944 | 0.000 | 0.000 | 0.000 | 1.239 | 0.085 | 104.503 |
| KTDKVSGSPHSKAQNQQT | 3375 | 60.935 | 0.000 | 0.015 | 0.000 | 0.765 | 0.128 | 146.657 |
| KTITGSGSPHSKAQTQLT | 3376 | 60.846 | 0.160 | 8.992 | 0.000 | 0.000 | 0.000 | 55.640 |
| KTINGSGSPHSKAQAPST | 3377 | 60.696 | 0.200 | 0.005 | 0.000 | 0.751 | 0.263 | 115.528 |
| KNCVGSGSPHSKAQNQQT | 3378 | 60.535 | 0.000 | 0.000 | 0.000 | 0.018 | 0.282 | 96.175 |
| KTIRDAGSPHSKAQNQQT | 3558 | 60.346 | 0.000 | 0.000 | 0.000 | 0.141 | 0.251 | 113.179 |
| KTVKDSGSPHSKAQNQQT | 3379 | 60.216 | 0.000 | 0.019 | 0.000 | 0.443 | 0.251 | 87.334 |
| KNALGSGSPHSKAQNQQT | 3380 | 60.014 | 0.000 | 0.003 | 0.000 | 0.682 | 0.213 | 137.222 |
| KVINGSGSPHSKGQNQQT | 3381 | 60.001 | 0.000 | 0.000 | 0.031 | 0.264 | 0.157 | 68.532 |
| KTVNGGGSPHSKAQNQQS | 3559 | 59.871 | 0.062 | 0.020 | 0.000 | 0.080 | 0.185 | 61.847 |
| KTIQDGGSPHSKAQNQQT | 3560 | 59.865 | 0.000 | 0.000 | 0.116 | 1.435 | 0.789 | 87.522 |
| KTISGGGSPHSKAQNQQN | 3561 | 59.801 | 0.000 | 0.000 | 0.000 | 0.722 | 0.039 | 87.761 |
| KTSNASGSPHSKAHNQQT | 3382 | 59.607 | 0.000 | 0.078 | 0.067 | 0.031 | 0.050 | 67.967 |
| KTINGSGSPHSKAQNTYA | 3383 | 59.603 | 0.000 | 0.000 | 0.000 | 0.425 | 0.346 | 101.715 |
| KTINGSGSPHSKSQNQHI | 3384 | 59.438 | 0.000 | 0.099 | 0.000 | 0.111 | 0.108 | 76.025 |
| KTINGGGSPHSKAQDKQT | 3562 | 59.322 | 0.000 | 0.000 | 0.000 | 0.000 | 0.093 | 50.764 |
| KTEFVSGSPHSKAQNQQT | 3385 | 59.306 | 0.000 | 0.000 | 0.000 | 0.196 | 0.276 | 69.788 |
| KTVNGSGSPHSKAQNHLT | 3386 | 59.239 | 0.133 | 0.034 | 0.000 | 0.000 | 0.156 | 70.786 |
| KTREISGSPHSKAQNQQT | 3387 | 59.027 | 0.000 | 0.042 | 0.224 | 0.356 | 0.269 | 51.696 |
| KTINGSGSPHSKAQIGMT | 3388 | 59.013 | 0.081 | 106.528 | 0.000 | 1.003 | 0.248 | 134.585 |
| KTIDGSGSPHSKALNKQT | 3389 | 58.992 | 0.000 | 0.267 | 0.000 | 0.000 | 0.056 | 74.626 |
| KTIIGGGSPHSKAQNPQT | 3563 | 58.924 | 0.000 | 0.202 | 0.000 | 0.000 | 0.126 | 53.992 |
| KQGEGSGSPHSKAQNQQT | 3390 | 58.752 | 0.000 | 0.000 | 0.000 | 0.000 | 0.151 | 135.300 |
| KTINGTGSPHSKAPNQLT | 3564 | 58.738 | 0.000 | 0.000 | 0.000 | 0.229 | 0.035 | 86.939 |
| KTVNGSGSPHSKAQLQQT | 3391 | 58.681 | 0.315 | 0.465 | 0.045 | 0.529 | 0.333 | 81.201 |
| KTFNGGGSPHSKAQYQQT | 3565 | 58.609 | 0.000 | 0.000 | 0.000 | 0.163 | 0.045 | 72.618 |
| KSINGSGSPHSKTQSQQT | 3392 | 58.608 | 0.000 | 3.017 | 0.000 | 0.155 | 0.017 | 71.397 |
| KTVNGGGSPHSKAQHQQT | 3566 | 58.602 | 0.729 | 0.000 | 0.000 | 0.000 | 0.043 | 138.544 |
| KSEKGSGSPHSKAQNQQT | 3393 | 58.566 | 0.000 | 0.010 | 0.011 | 1.601 | 0.059 | 158.931 |
| KNVNGSGSPHSKAQNQQT | 3394 | 58.481 | 0.000 | 0.000 | 0.000 | 0.917 | 0.166 | 53.379 |
| KGGEGSGSPHSKAQNQQT | 3395 | 58.472 | 0.000 | 0.034 | 0.000 | 0.037 | 0.066 | 91.023 |
| KTINGSGSPHSKAQRMST | 3396 | 58.435 | 0.192 | 0.037 | 0.000 | 1.707 | 0.882 | 53.414 |
| KTINGSGSPHSKAQGILT | 3397 | 58.418 | 0.000 | 0.005 | 0.010 | 0.569 | 0.192 | 102.631 |
| KEFVGSGSPHSKAQNQQT | 3398 | 58.374 | 0.000 | 0.046 | 0.000 | 0.088 | 0.326 | 128.675 |
| KTIIGSGSPHSKAQDRQT | 3399 | 58.258 | 1.393 | 0.230 | 0.219 | 0.000 | 0.045 | 53.981 |
| KSDKGSGSPHSKAQNQQT | 3400 | 58.248 | 0.000 | 0.000 | 0.000 | 0.076 | 0.166 | 146.566 |
| KTEQVSGSPHSKAQNQQT | 3401 | 58.247 | 0.000 | 0.000 | 0.000 | 0.000 | 0.081 | 88.487 |
| KTEHVSGSPHSKAQNQQT | 3402 | 58.228 | 0.000 | 0.024 | 0.000 | 0.433 | 0.141 | 71.410 |
| KTINGSGSPHSKARDWQT | 3403 | 58.216 | 0.000 | 0.005 | 0.000 | 0.800 | 0.259 | 120.704 |
| KTENASGSPHSKAQNQQT | 3404 | 58.187 | 0.000 | 0.038 | 0.000 | 0.371 | 0.129 | 88.439 |
| KEVQGSGSPHSKAQNQQT | 3405 | 58.125 | 0.000 | 0.000 | 0.000 | 0.657 | 0.000 | 168.220 |
| KTINGSGSPHSKAQNTHD | 3406 | 58.108 | 0.000 | 0.027 | 0.000 | 0.410 | 0.126 | 81.189 |
| KTINGSGSPHSKAPNLQI | 3407 | 58.022 | 0.000 | 0.044 | 0.000 | 1.548 | 0.243 | 55.714 |
| KTINGSGSPHSKAQERST | 3408 | 58.021 | 0.000 | 0.011 | 0.005 | 0.829 | 0.409 | 87.656 |
| KTSNGSGSPHSKAQNYQT | 3409 | 57.894 | 0.000 | 0.082 | 0.000 | 0.000 | 0.110 | 63.681 |
| KTEYISGSPHSKAQNQQT | 3410 | 57.891 | 0.000 | 0.000 | 0.000 | 0.076 | 0.075 | 57.620 |
| KTINGSGSPHSKAQRTCT | 3411 | 57.863 | 0.000 | 0.140 | 0.129 | 1.855 | 1.716 | 90.146 |
| KTINGSGSPHSKAQIGHT | 3412 | 57.769 | 0.024 | 0.000 | 0.000 | 0.281 | 0.154 | 99.262 |
| KNCWGSGSPHSKAQNQQT | 3413 | 57.756 | 0.000 | 0.000 | 0.000 | 0.000 | 0.092 | 59.888 |
| KTINGSGSPHSKAQGAIT | 3414 | 57.627 | 0.000 | 0.000 | 0.000 | 0.594 | 0.161 | 95.696 |
| KTDVNSGSPHSKAQNQQT | 3415 | 57.593 | 0.000 | 0.000 | 0.000 | 0.000 | 0.331 | 66.127 |
| KSDIGSGSPHSKAQNQQT | 3416 | 57.592 | 0.000 | 0.000 | 0.000 | 0.844 | 0.128 | 107.342 |
| KTINGSGSPHSKAQVPPT | 3417 | 57.316 | 0.000 | 0.012 | 0.000 | 0.257 | 0.200 | 90.220 |
| KTINGSGSPHSKAQVQQI | 3418 | 57.308 | 0.000 | 1.113 | 0.000 | 0.000 | 0.113 | 61.957 |
| KTINGSGSPHSKALMRQT | 3419 | 57.234 | 0.060 | 0.036 | 0.100 | 1.798 | 0.517 | 81.332 |
| KTINGSGSPHSKAQYSVT | 3420 | 57.130 | 0.000 | 0.093 | 0.000 | 1.235 | 0.302 | 60.023 |
| KNSIGSGSPHSKAQNQQT | 3421 | 57.101 | 0.000 | 0.052 | 0.000 | 0.083 | 0.074 | 97.381 |
| KTINGSGSPHSKVPNLQT | 3422 | 57.046 | 0.000 | 0.029 | 0.000 | 0.459 | 0.082 | 50.474 |
| KAINGSGSPHSKAQSQQI | 3423 | 56.976 | 0.000 | 0.000 | 0.000 | 0.000 | 0.000 | 57.052 |
| KTINGSGSPHSKAQAITT | 3424 | 56.924 | 0.000 | 0.000 | 0.000 | 1.239 | 0.438 | 75.250 |
| KTINGSGSPHSKAQKTLT | 3425 | 56.844 | 0.000 | 0.017 | 0.009 | 1.800 | 1.400 | 66.415 |
| KTVNGSGSPHSKAQNQWT | 3426 | 56.823 | 0.000 | 0.000 | 0.299 | 0.000 | 0.219 | 69.906 |
| KTINGSGSPHSKAQLHHT | 3427 | 56.815 | 0.025 | 0.000 | 0.010 | 0.712 | 0.368 | 58.418 |
| KTEQTSGSPHSKAQNQQT | 3428 | 56.683 | 0.000 | 0.045 | 0.000 | 0.792 | 0.430 | 59.360 |
| KTINGSGSPHSKAQNIII | 3429 | 56.630 | 0.000 | 0.062 | 0.123 | 0.099 | 0.056 | 76.742 |
| KNSLGSGSPHSKAQNQQT | 3430 | 56.621 | 0.000 | 0.028 | 0.000 | 0.308 | 0.162 | 101.942 |
| KTIPMEGSPHSKAQNQQT | 3567 | 56.560 | 0.000 | 0.000 | 0.000 | 1.824 | 0.371 | 89.951 |
| KTINGSGSPHSKAQGHHT | 3431 | 56.559 | 0.000 | 0.000 | 0.000 | 0.632 | 0.117 | 71.050 |
| KTDRTSGSPHSKAQNQQT | 3432 | 56.466 | 0.000 | 0.000 | 0.000 | 0.062 | 0.160 | 148.498 |
| KTINGSGSPHSKAQSKVT | 3433 | 56.373 | 0.000 | 0.050 | 0.014 | 1.021 | 0.390 | 76.115 |
| KEVVGSGSPHSKAQNQQT | 3434 | 56.371 | 0.000 | 0.000 | 0.000 | 0.000 | 0.323 | 116.964 |
| KTINGSGSPHSKAQLPST | 3435 | 56.238 | 0.005 | 4.258 | 0.001 | 1.040 | 0.185 | 84.918 |
| KTINGSGSPHSKAIGKQT | 3436 | 56.158 | 0.000 | 0.000 | 0.000 | 0.887 | 0.088 | 110.132 |
| KTEPTSGSPHSKAQNQQT | 3437 | 56.134 | 0.000 | 0.000 | 0.000 | 0.061 | 0.527 | 143.397 |
| KTVNGGGSPHSKSQNQQT | 3568 | 56.114 | 0.116 | 0.000 | 0.000 | 0.000 | 0.040 | 170.548 |
| KTINGSGSPHSKAQAIHT | 3438 | 56.047 | 0.000 | 0.000 | 212.32 | 0.887 | 0.890 | 81.908 |
| KTINGSGSPHSKAQHGLT | 3439 | 55.999 | 0.000 | 0.000 | 0.101 | 1.913 | 0.244 | 117.191 |
| KSELGSGSPHSKAQNQQT | 3440 | 55.997 | 0.000 | 0.005 | 0.000 | 0.881 | 0.239 | 120.521 |
| KTINGSGSPHSKAQFMCT | 3441 | 55.916 | 0.000 | 0.000 | 0.000 | 0.078 | 0.448 | 81.959 |
| KTINVSGSPHSKAQGQQT | 3442 | 55.870 | 0.000 | 0.191 | 0.000 | 0.592 | 0.040 | 87.211 |
| KTINGGGSPHSKAQNQMT | 3569 | 55.778 | 0.000 | 0.000 | 0.000 | 0.866 | 0.012 | 73.177 |
| KTVNGSGSPHSKAQHLQT | 3443 | 55.739 | 0.091 | 0.036 | 0.000 | 0.062 | 0.409 | 62.743 |
| KTIRENGSPHSKAQNQQT | 3570 | 55.605 | 0.000 | 0.000 | 0.016 | 0.131 | 0.257 | 95.931 |
| KTINGSGSPHSKTQNHQN | 3444 | 55.551 | 0.000 | 0.048 | 0.000 | 0.000 | 0.099 | 64.846 |
| KTINGSGSPHSKAQPART | 3445 | 55.513 | 0.000 | 0.000 | 0.328 | 1.294 | 0.991 | 127.301 |
| KTVNGSGSPHSKAQSLQT | 3446 | 55.497 | 0.000 | 0.060 | 0.000 | 0.000 | 0.143 | 69.033 |
| KTINGSGSPHSKSQSQLT | 3447 | 55.430 | 0.000 | 0.035 | 0.000 | 0.050 | 0.013 | 125.577 |
| KTINGSASPHSKAHSQQT | 3571 | 55.293 | 0.000 | 0.000 | 0.000 | 0.000 | 0.166 | 66.252 |
| KTWQNSGSPHSKAQNQQT | 3448 | 55.245 | 0.000 | 0.000 | 0.000 | 0.111 | 0.265 | 114.258 |
| KTINGSGSPHSKAQDRQS | 3449 | 55.137 | 1.146 | 0.016 | 0.106 | 0.644 | 0.086 | 55.701 |
| KTINGSGSPHSKAQMPST | 3450 | 54.986 | 1.691 | 0.039 | 0.028 | 0.450 | 0.202 | 114.331 |
| KTNNGGGSPHSKAQNLQT | 3572 | 54.963 | 0.000 | 0.000 | 0.000 | 0.000 | 0.089 | 80.506 |
| KTINGSGSPHSKAQGSLT | 3451 | 54.717 | 0.000 | 0.006 | 0.013 | 0.480 | 0.298 | 142.786 |
| KTEVTSGSPHSKAQNQQT | 3452 | 54.663 | 0.000 | 0.000 | 0.000 | 0.323 | 0.185 | 81.482 |
| KSINGGGSPHSKAQYQQT | 3573 | 54.612 | 0.000 | 0.000 | 0.000 | 0.105 | 0.010 | 65.952 |
| KTVIGSGSPHSKSQNQQT | 3453 | 54.603 | 0.000 | 0.000 | 0.000 | 0.000 | 0.106 | 69.121 |
| KAVNVSGSPHSKAQNQQT | 3454 | 54.586 | 0.000 | 0.000 | 0.000 | 0.000 | 0.023 | 57.835 |
| KTVNGNGSPHSKSQNQQT | 3574 | 54.586 | 0.000 | 0.000 | 0.000 | 0.256 | 0.168 | 95.384 |
| KTDRNSGSPHSKAQNQQT | 3455 | 54.495 | 0.000 | 0.000 | 0.000 | 0.823 | 0.241 | 85.823 |
| KTINGSGSPHSKAQVPAT | 3456 | 54.475 | 0.000 | 0.002 | 0.000 | 0.782 | 0.223 | 137.743 |
| KGVLGSGSPHSKAQNQQT | 3457 | 54.472 | 0.000 | 0.007 | 0.027 | 0.359 | 0.189 | 145.740 |
| KTLNGNGSPHSKAQNLQT | 3575 | 54.458 | 0.668 | 0.000 | 0.000 | 0.161 | 0.172 | 159.134 |
| KAINGSGSPHSKAQDKQT | 3458 | 54.452 | 0.000 | 0.000 | 0.057 | 0.044 | 0.223 | 56.004 |
| KTSNGSGSPHSKAHYQQT | 3459 | 54.414 | 0.000 | 0.251 | 0.000 | 0.249 | 0.204 | 54.162 |
| KTINGSGSPHSKAQVPST | 3460 | 54.366 | 0.000 | 1.001 | 0.000 | 0.202 | 0.139 | 117.223 |
| KTINGSGSSHSKAQNQQT | 3576 | 54.292 | 1.709 | 1.870 | 1.287 | 1.075 | 0.458 | 67.731 |
| KTELRSGSPHSKAQNQQT | 3461 | 54.289 | 0.000 | 0.007 | 0.040 | 0.790 | 0.239 | 57.814 |
| KNINGSGSPHSKAQNHQT | 3462 | 54.248 | 0.000 | 0.034 | 0.000 | 0.340 | 0.075 | 74.979 |
| KTVNGGGSPHSKAQNHQT | 3577 | 54.246 | 0.375 | 0.024 | 0.000 | 0.000 | 0.146 | 67.188 |
| KTINGSGSPHSKARGEQT | 3463 | 54.207 | 0.025 | 0.006 | 0.005 | 0.309 | 0.327 | 128.098 |
| KTINGGGSPHSKAQYQHT | 3578 | 54.188 | 0.000 | 0.000 | 0.000 | 0.000 | 0.223 | 82.256 |
| KTEDLSGSPHSKAQNQQT | 3464 | 54.156 | 0.000 | 0.000 | 0.000 | 1.193 | 0.132 | 70.198 |
| KTINGSGSPHSKAPGQQT | 3465 | 54.071 | 0.065 | 0.000 | 0.004 | 0.542 | 0.179 | 73.440 |
| KTIPKNGSPHSKAQNQQT | 3579 | 53.824 | 0.000 | 0.032 | 0.000 | 0.115 | 0.178 | 77.458 |
| KTINGSGSPHSKAQSLQI | 3466 | 53.778 | 0.000 | 0.186 | 0.000 | 0.022 | 0.047 | 51.543 |
| KTINGSGSPHSKRLEQQT | 3467 | 53.512 | 0.000 | 0.118 | 0.003 | 0.161 | 0.292 | 71.704 |
| KTERGSGSPHSKAQNQQT | 3468 | 53.475 | 0.000 | 0.030 | 0.000 | 1.416 | 0.175 | 85.368 |
| KTVNGSGSPHSKAPNQQT | 3469 | 53.444 | 0.833 | 2.206 | 0.006 | 0.156 | 0.178 | 58.080 |
| KTSNGSGSPHSKAQNQST | 3470 | 53.353 | 0.000 | 0.000 | 0.000 | 0.000 | 0.014 | 120.897 |
| KTINGSGSPHSKAQKVIT | 3471 | 53.273 | 0.000 | 0.000 | 0.000 | 0.357 | 0.402 | 95.147 |
| KTEGISGSPHSKAQNQQT | 3472 | 53.270 | 0.000 | 0.000 | 0.000 | 0.000 | 0.010 | 78.303 |
| KTINGSGSPHSKAQNNDQ | 3473 | 53.226 | 0.000 | 0.000 | 0.000 | 0.593 | 0.046 | 59.664 |
| KTINGSGSPHSKAQSVHT | 3474 | 53.226 | 0.000 | 0.004 | 0.000 | 0.446 | 0.217 | 76.110 |
| KTINGSGSPHSKAQPLGT | 3475 | 53.049 | 0.015 | 0.004 | 0.001 | 0.515 | 0.222 | 68.656 |
| KTINKEGSPHSKAQNQQT | 3580 | 53.006 | 0.000 | 0.029 | 0.000 | 0.177 | 0.111 | 64.520 |
| KTCNASGSPHSKAQNQQT | 3476 | 52.998 | 0.000 | 0.011 | 0.000 | 0.897 | 0.141 | 67.934 |
| KAINGSGSPHSKAHNQET | 3477 | 52.973 | 0.000 | 0.030 | 0.000 | 0.035 | 0.058 | 71.809 |
| KTEGLSGSPHSKAQNQQT | 3478 | 52.891 | 0.000 | 0.000 | 0.020 | 0.104 | 0.155 | 104.529 |
| KTRDASGSPHSKAQNQQT | 3479 | 52.861 | 0.000 | 0.000 | 0.010 | 1.062 | 0.402 | 52.089 |
| KTSNGSGSPHSKAQNLQI | 3480 | 52.843 | 0.000 | 0.000 | 1.605 | 0.178 | 0.214 | 74.823 |
| KTGNGSGSPHSKAQIQQT | 3481 | 52.809 | 0.000 | 0.000 | 0.000 | 0.000 | 0.012 | 98.291 |
| KTVNGGGSPHSKAQNLQT | 3581 | 52.788 | 0.000 | 0.031 | 0.000 | 0.000 | 0.165 | 83.215 |
| KTDRSSGSPHSKAQNQQT | 3482 | 52.737 | 0.000 | 0.000 | 0.000 | 0.995 | 0.085 | 123.421 |
| KTINGSGSPHSKAQVRNT | 3483 | 52.735 | 0.000 | 0.101 | 0.011 | 0.230 | 0.423 | 68.893 |
| KTINGSGSPHSKAPSNQT | 3484 | 52.680 | 1.494 | 4.762 | 0.003 | 0.330 | 0.208 | 87.951 |
| KTINGSGSPHSKAQVGHT | 3485 | 52.624 | 0.000 | 0.000 | 0.006 | 0.535 | 0.192 | 106.448 |
| KNAIGSGSPHSKAQNQQT | 3486 | 52.516 | 0.000 | 0.000 | 0.000 | 0.165 | 0.198 | 117.939 |
| KAENGSGSPHSKAQNQQT | 3487 | 52.487 | 0.000 | 0.157 | 0.029 | 0.000 | 0.242 | 120.256 |
| KTINGSGSPHSKAQRDIT | 3488 | 52.415 | 0.098 | 0.000 | 0.008 | 1.784 | 0.605 | 88.122 |
| KTINGSGSPHSKAQMPNT | 3489 | 52.408 | 0.084 | 0.036 | 0.025 | 0.057 | 0.359 | 66.040 |
| KTVNGSGSPHSKSQNQQT | 3490 | 52.395 | 0.033 | 0.077 | 0.013 | 0.105 | 0.175 | 58.000 |
| KTIPAIGSPHSKAQNQQT | 3582 | 52.346 | 0.000 | 0.009 | 0.000 | 0.034 | 0.134 | 51.949 |
| KTINGSGSPHSKARGLQT | 3491 | 52.275 | 0.000 | 0.000 | 0.036 | 1.235 | 1.425 | 169.881 |
| KTELGSGSPHSKAQNQQT | 3492 | 52.232 | 0.000 | 0.007 | 0.006 | 0.532 | 0.088 | 87.314 |
| KAETGSGSPHSKAQNQQT | 3493 | 52.219 | 0.000 | 0.047 | 0.581 | 0.009 | 0.188 | 132.940 |
| KTINGSGSPHSKLQKQQT | 3494 | 52.144 | 0.615 | 0.477 | 1.071 | 1.113 | 0.429 | 61.833 |
| KTINGSGSPHSKAPSLQT | 3495 | 52.137 | 0.041 | 1.614 | 0.002 | 0.902 | 0.222 | 70.363 |
| KTINGSGSPHSKAQRDQT | 3496 | 51.897 | 0.069 | 0.014 | 0.040 | 0.867 | 0.554 | 102.317 |
| KTDVGSGSPHSKAQNQQT | 3497 | 51.849 | 0.000 | 0.007 | 0.000 | 0.385 | 0.560 | 115.774 |
| KTINGSGSPHSKNRDQQT | 3498 | 51.830 | 0.000 | 0.008 | 0.000 | 0.480 | 0.138 | 100.300 |
| KSINGSGSPHSKAPNLQT | 3499 | 51.812 | 0.000 | 0.256 | 0.000 | 0.085 | 0.139 | 59.270 |
| KTINGSGSPHSKAQAKGT | 3500 | 51.727 | 0.048 | 0.016 | 0.000 | 0.271 | 0.525 | 104.917 |
| KTVNGSGSPHSKAQDKQT | 3501 | 51.580 | 0.428 | 0.000 | 0.069 | 0.041 | 0.063 | 69.225 |
| KTINGGGSPHSKAQNPQA | 3583 | 51.574 | 0.000 | 0.000 | 0.000 | 0.192 | 0.000 | 102.792 |
| KTINGSGSPHSKAQSAHT | 3502 | 51.569 | 0.068 | 0.070 | 0.000 | 0.589 | 0.249 | 79.498 |
| KTINGNGSPHSKSQNQHT | 3584 | 51.379 | 0.000 | 0.054 | 0.000 | 0.000 | 0.082 | 56.614 |
| KTVPTSGSPHSKAQNQQT | 3503 | 51.348 | 0.013 | 0.000 | 0.000 | 1.017 | 0.338 | 102.651 |
| KTIDGSGSPHSKSQNHQT | 3504 | 51.307 | 0.000 | 0.000 | 0.000 | 0.000 | 0.269 | 63.174 |
| KTDVKSGSPHSKAQNQQT | 3505 | 51.296 | 0.000 | 0.000 | 0.000 | 0.515 | 0.224 | 53.601 |
| KAINRSGSPHSKAQDQQT | 3506 | 51.262 | 0.000 | 0.000 | 0.000 | 0.000 | 0.036 | 54.631 |
| KTINGSGSPHSKAQSTMT | 3507 | 51.249 | 0.018 | 0.002 | 0.002 | 0.321 | 0.341 | 73.213 |
| KTVNASGSPHSKAQNQLT | 3508 | 51.249 | 0.000 | 0.000 | 0.000 | 0.000 | 0.268 | 99.559 |
| KTINGSGSPHSKAQREMT | 3509 | 51.076 | 0.000 | 24.900 | 143.49 | 1.564 | 0.476 | 70.961 |
| KTVHGSGSPHSKAQSQQT | 3510 | 51.057 | 0.000 | 0.000 | 0.000 | 0.143 | 0.146 | 54.185 |
| KTINGGGSPHSKSQNRQT | 3585 | 51.017 | 0.000 | 0.000 | 0.000 | 0.000 | 0.421 | 149.370 |
| KTINGSGSPHSKAQYRAT | 3511 | 51.008 | 0.000 | 0.158 | 0.000 | 0.690 | 0.120 | 50.650 |
| KTINGGGSPHSKAQRQQT | 3586 | 50.998 | 0.000 | 0.041 | 0.000 | 0.991 | 0.142 | 147.942 |
| KTEPMSGSPHSKAQNQQT | 3512 | 50.960 | 0.203 | 0.000 | 0.000 | 1.816 | 0.415 | 126.322 |
| KTINGSGSPHSKNQWQQT | 3513 | 50.800 | 0.000 | 0.044 | 0.047 | 0.111 | 0.324 | 65.506 |
| KETAGSGSPHSKAQNQQT | 3514 | 50.762 | 0.000 | 0.027 | 0.000 | 1.706 | 0.054 | 212.795 |
| KTINGSGSPHSKAQRMNT | 3515 | 50.686 | 0.000 | 108.747 | 0.019 | 0.943 | 0.264 | 97.975 |
| KNNLGSGSPHSKAQNQQT | 3516 | 50.670 | 0.000 | 0.019 | 0.000 | 0.406 | 0.121 | 102.408 |
| KTINGSGSPHAKAQNHQT | 3517 | 50.667 | 0.211 | 0.140 | 0.051 | 0.101 | 0.090 | 80.603 |
| KTIIKNGSPHSKAQNQQT | 3587 | 50.587 | 0.000 | 0.000 | 0.000 | 0.000 | 0.751 | 75.547 |
| KTINGSGSPHSYHVNQQT | 3588 | 50.486 | 0.000 | 0.056 | 0.059 | 0.528 | 0.275 | 179.489 |
| KTINGSGSPHSKAGDSQT | 3518 | 50.457 | 0.614 | 0.236 | 0.008 | 1.062 | 0.071 | 74.355 |
| KTINGSGSPHSKLKSQQT | 3519 | 50.368 | 0.000 | 0.296 | 0.000 | 1.796 | 1.096 | 95.240 |
| KTINGSGSPHSKAQKIST | 3520 | 50.285 | 0.000 | 0.000 | 0.088 | 0.108 | 0.302 | 51.115 |
| KTEYNSGSPHSKAQNQQT | 3521 | 50.256 | 0.000 | 0.000 | 0.000 | 0.000 | 0.009 | 62.679 |
| KTINGSGSPHSKAPSMQT | 3522 | 50.249 | 0.000 | 0.000 | 0.004 | 0.941 | 0.460 | 75.504 |
| EAINGSGSPHSKAQNQQT | 3523 | 50.243 | 0.629 | 0.094 | 0.000 | 0.057 | 1.519 | 117.305 |
| KTINGSGSPHSKASPRQT | 3524 | 50.227 | 0.088 | 0.005 | 0.068 | 1.761 | 0.530 | 67.241 |
| KTINGSGSPHSKRMEQQT | 3525 | 50.177 | 0.000 | 0.000 | 0.000 | 1.327 | 0.208 | 81.769 |
| KTINGSGSPHSKAQYQNT | 3526 | 50.099 | 0.000 | 0.008 | 0.000 | 0.017 | 0.119 | 71.846 |
| KTERVSGSPHSKAQNQQT | 3589 | 96.943 | 0.000 | 0.000 | 0.000 | 0.000 | 0.144 | 135.438 |
| KAEIGHDSPHKSGQNQQT | 1754 | 63.249 | 0.000 | 0.000 | 0.000 | 0.060 | 0.024 | 27.173 |

Table 26 provides the peptide sequence of 341 matured capsid variants, and the fold enrichment of these matured capsid variants relative to the AAV9 control that demonstrated a 75-fold or greater increase in expression in the brain of NHPs relative to the AAV9 control and had a fold-change in expression that was less than 2 relative to the AAV9 control in the liver and the DRG.

TABLE 26
NGS fold-enrichment of TTM-001 and TTM-002 matured AAV capsid
variants in the brain of NHPs

| | | Fold Enrichment relative to AAV9 | | | | | | |
|---|---|---|---|---|---|---|---|---|
| | SEQ | | | | | Liver | Liver | |
| | ID | Brain | DRG | Heart | Muscle | RNA | DNA | Brain |
| Sequence | NO: | (NHP) | (NHP) | (NHP) | (NHP) | (NHP) | (NHP) | (Mouse) |
| KTFNRSGSPHSKAQNQQI | 3591 | 86.359 | 0.000 | 113.67 | 0.000 | 0.000 | 0.092 | 25.568 |
| KTIIGSGSPHSKAQNRHT | 3239 | 217.176 | 0.000 | 0.000 | 0.000 | 0.000 | 0.000 | 210.515 |
| KTFPGSGSPHSKVONQQT | 3240 | 199.720 | 0.000 | 0.000 | 0.000 | 0.000 | 0.967 | 97.703 |
| KTEKMSGSPHSKAQNOQT | 3241 | 169.461 | 0.523 | 0.000 | 0.000 | 0.000 | 0.158 | 109.161 |
| KAINGHDSPHKSGQIRQT | 3606 | 108.510 | 0.000 | 23.908 | 0.000 | 0.132 | 0.261 | 8.862 |
| KTINGHDSPHKIGONQHA | 3607 | 77.321 | 0.000 | 18.836 | 0.028 | 0.220 | 0.132 | 7.578 |
| KEINGRGSPHSKAQNOOT | 3527 | 134.390 | 0.239 | 0.000 | 0.000 | 0.000 | 0.232 | 52.311 |
| KTVNRNGSPHSKAQNQQT | 3528 | 133.016 | 0.000 | 0.416 | 0.000 | 0.000 | 0.000 | 85.361 |
| KAINGYDSPHKSGQKQQT | 3608 | 83.803 | 0.041 | 9.491 | 0.000 | 0.031 | 0.150 | 13.057 |
| KTVNGSGSPHSKARDQQT | 3242 | 124.789 | 0.123 | 0.039 | 0.312 | 0.569 | 0.454 | 132.137 |
| KTESGHDSPHKSGONOQT | 3609 | 86.513 | 0.000 | 7.414 | 0.000 | 0.000 | 0.038 | 13.163 |
| KTINGHDSPHKSGQSVQT | 3610 | 75.748 | 0.010 | 6.808 | 0.000 | 0.165 | 0.058 | 9.321 |
| KTFNGSGSPHSKAPNLQT | 3243 | 121.436 | 0.000 | 0.167 | 0.000 | 0.000 | 0.015 | 168.920 |
| KTEKTSGSPHSKAQNQQT | 3244 | 120.337 | 0.000 | 0.355 | 0.000 | 0.000 | 0.119 | 101.467 |
| TTINGHDSPHKSGONOQT | 3611 | 108.963 | 1.512 | 3.445 | 0.869 | 0.659 | 1.109 | 14.788 |
| KTINGHESPHKSGRSQQT | 3612 | 97.106 | 0.000 | 3.329 | 0.022 | 0.000 | 0.181 | 9.378 |
| KTINGSGSPHSKAHVRQT | 3245 | 119.798 | 0.000 | 0.000 | 0.262 | 0.694 | 1.039 | 165.590 |
| KTVNGSGSPHSKAPNQHT | 3246 | 117.207 | 0.000 | 0.109 | 0.000 | 0.000 | 0.074 | 51.008 |
| KTEKISGSPHSKAQNQQT | 3247 | 116.603 | 0.000 | 0.000 | 0.000 | 0.000 | 0.426 | 102.978 |
| KTINGPGSPHSKAHNQQT | 3529 | 115.742 | 0.146 | 0.000 | 0.235 | 0.000 | 0.513 | 52.508 |
| KTINGHDSPHKSGONKLE | 3613 | 76.204 | 0.000 | 1.430 | 0.000 | 0.015 | 0.031 | 12.419 |
| KTVNGSGSPHSKTQSQQT | 3248 | 115.086 | 0.000 | 0.726 | 0.000 | 0.000 | 0.340 | 63.248 |
| TTINGSGSPHSKAQNQQT | 3249 | 114.856 | 1.340 | 14.856 | 0.827 | 1.281 | 0.957 | 72.058 |
| KSINESGSPHSKAQNQQI | 3250 | 113.833 | 0.000 | 0.000 | 0.000 | 0.000 | 0.000 | 67.649 |
| KTINGHDSPHKTGONQQK | 3614 | 77.562 | 0.000 | 1.056 | 0.000 | 0.000 | 0.000 | 6.379 |
| KTERTSGSPHSKAQNQQT | 3251 | 112.957 | 0.000 | 0.009 | 0.000 | 1.128 | 0.207 | 117.374 |
| KTINGSGSPHSKAQPAKT | 3252 | 111.472 | 0.331 | 0.000 | 1.089 | 0.044 | 1.796 | 215.275 |
| KTINGRGSPHKRGONOQT | 3837 | 120.889 | 0.100 | 0.814 | 0.434 | 0.458 | 0.614 | 13.988 |
| KTINGSGSPHTKAQNPPT | 3592 | 147.061 | 0.000 | 0.727 | 0.000 | 0.000 | 0.000 | 34.425 |
| KTEKSSGSPHSKAQNQQT | 3253 | 107.470 | 0.000 | 0.016 | 0.014 | 0.977 | 0.179 | 100.177 |
| KAINGHDNPHKSGONOQT | 3615 | 88.906 | 0.297 | 0.721 | 0.482 | 0.222 | 0.130 | 9.702 |
| KTSYGNGSPHSKAQNOOT | 3530 | 105.937 | 0.000 | 0.000 | 0.000 | 0.000 | 0.114 | 105.894 |
| KTINGQDSPHKSGQHQQA | 3616 | 85.657 | 1.127 | 0.579 | 0.000 | 0.193 | 0.557 | 5.582 |
| KTEKGSGSPHSKAQNOQT | 3254 | 105.614 | 0.053 | 0.031 | 0.000 | 0.586 | 0.169 | 84.653 |
| KTINGSGSPHSKSQTQQN | 3255 | 104.474 | 0.000 | 0.131 | 0.000 | 0.084 | 0.038 | 54.021 |
| KTERISGSPHSKAQNOQT | 3256 | 103.692 | 0.000 | 0.000 | 0.000 | 0.062 | 0.370 | 89.637 |
| KTERASGSPHSKAQNQQT | 3257 | 103.669 | 0.000 | 0.000 | 0.000 | 0.127 | 0.070 | 115.550 |
| KSINGHDSPHKSGOIQHT | 3617 | 87.598 | 0.000 | 0.480 | 0.000 | 0.714 | 0.347 | 13.872 |
| KELHGSGSPHSKAQNOQT | 3258 | 102.680 | 0.000 | 0.000 | 0.000 | 1.634 | 0.592 | 96.554 |
| KAINGSGSPHSKAONLAT | 3259 | 101.954 | 0.000 | 10.954 | 8.655 | 0.298 | 0.239 | 116.685 |
| KTVNGSGSPHSKSQNQLT | 3260 | 101.327 | 0.000 | 0.035 | 0.000 | 0.000 | 0.025 | 80.716 |
| KAINGHDSPHKSGPROQT | 3618 | 145.142 | 0.000 | 0.408 | 0.000 | 0.000 | 0.000 | 8.259 |
| KTVNGHDSPHKSGHTOQT | 3619 | 82.246 | 0.000 | 0.378 | 1.142 | 0.000 | 0.123 | 6.160 |
| KSINGHDSPHKSGORQHT | 3620 | 80.132 | 0.000 | 0.357 | 0.000 | 0.000 | 0.000 | 9.851 |
| KTERNSGSPHSKAQNOQT | 3261 | 99.892 | 0.000 | 0.000 | 0.000 | 0.000 | 0.107 | 87.392 |
| KSLNGSGSPHTKAQNOQT | 3593 | 81.515 | 0.197 | 0.333 | 0.000 | 0.000 | 0.085 | 45.140 |
| KSVNGNGSPHSKAQNQQT | 3531 | 99.385 | 0.000 | 1.329 | 0.000 | 0.359 | 0.079 | 51.016 |
| KAINGHDSPHKSAQSQQT | 3621 | 95.204 | 0.146 | 0.310 | 0.000 | 0.699 | 0.058 | 14.595 |
| KSIYGHESPHKSGONQQS | 3622 | 90.947 | 0.817 | 0.310 | 0.000 | 0.000 | 0.243 | 8.064 |
| KTFNGSGSPHSKAQGQQT | 3262 | 99.253 | 0.000 | 0.208 | 0.000 | 0.128 | 0.099 | 81.459 |
| KTVNGHDSPHKSLONQQT | 67 | 112.925 | 0.000 | 0.301 | 0.059 | 0.000 | 0.322 | 16.726 |
| KTINGSGSPHGWVONQQT | 3532 | 97.122 | 0.000 | 0.000 | 0.000 | 1.240 | 1.975 | 290.720 |
| KTINGHGSPHSKAQNPQT | 3838 | 83.478 | 0.000 | 0.288 | 0.219 | 0.000 | 0.260 | 11.001 |
| KTSNGYDSPHKSGOKQQT | 3624 | 77.001 | 0.032 | 0.286 | 0.000 | 0.000 | 0.016 | 8.813 |
| KTVNGHDSPHKSGRNQET | 3625 | 102.695 | 0.000 | 0.286 | 0.000 | 0.000 | 0.027 | 11.958 |
| KTTNGHDSPHKSGQTOLT | 3626 | 115.637 | 0.000 | 0.283 | 0.000 | 0.052 | 0.321 | 17.885 |
| KAINGHDSPHKSEKNOQT | 3627 | 77.103 | 0.000 | 0.274 | 0.000 | 0.000 | 0.000 | 26.868 |
| KTERVSGSPHSKAQNQQT | 3263 | 96.943 | 0.000 | 0.000 | 0.000 | 0.000 | 0.144 | 135.438 |
| KTINGSGSPHSKALNRQS | 3264 | 96.843 | 0.136 | 0.532 | 0.000 | 0.042 | 0.178 | 55.945 |
| KTERLSGSPHSKAQNOQT | 3265 | 95.857 | 0.000 | 0.004 | 0.005 | 0.126 | 0.260 | 102.372 |
| KIINGRDSPHKSGODQQT | 3628 | 78.773 | 0.000 | 0.254 | 0.000 | 0.000 | 0.156 | 16.132 |
| KTDNGSGSPHSKAHNQQT | 3266 | 95.164 | 0.000 | 0.000 | 0.000 | 0.000 | 0.027 | 55.313 |
| KTFHGSGSPHSKTONQQT | 3267 | 94.714 | 0.000 | 0.210 | 0.120 | 0.000 | 0.000 | 51.119 |
| KTISGHDSPHKTGHNQQT | 3629 | 92.490 | 0.000 | 0.233 | 0.057 | 0.730 | 0.000 | 8.823 |
| KTVNAHDSPHKSGONOLT | 3630 | 79.137 | 0.000 | 0.233 | 0.178 | 0.753 | 0.045 | 29.254 |
| KTINGGGSPHSKAQTQQI | 3533 | 92.345 | 0.000 | 0.000 | 0.000 | 0.000 | 0.023 | 54.199 |
| KSINGYDSPHKSGOTOQT | 3631 | 79.227 | 1.817 | 0.226 | 0.000 | 0.000 | 1.148 | 4.497 |
| KTINGHESPHKSGOTQQI | 3632 | 86.089 | 0.000 | 0.222 | 0.000 | 0.000 | 0.024 | 3.989 |
| KTINGHDSPHKSGQSKQA | 3633 | 101.460 | 0.000 | 0.222 | 0.000 | 0.185 | 0.114 | 7.510 |
| KTSNGSGSPHSKAQNPPT | 3268 | 91.528 | 0.000 | 0.000 | 0.000 | 0.000 | 0.039 | 51.541 |
| ETINGSGSPHSKAQNLQT | 3269 | 90.969 | 0.221 | 1.023 | 0.197 | 0.179 | 0.813 | 107.216 |
| KTVHGNGSPHSKAQNQQT | 3534 | 90.073 | 0.000 | 0.000 | 0.000 | 0.000 | 0.304 | 97.003 |
| NTINGSGSPHSKAQNQQT | 3270 | 90.017 | 1.712 | 1.261 | 1.171 | 0.923 | 0.540 | 55.179 |
| KTINGGGSPHSKAQNQQC | 3535 | 89.301 | 0.219 | 0.000 | 0.000 | 0.287 | 0.319 | 53.840 |
| KTENMSGSPHSKAQNQQT | 3271 | 89.247 | 0.000 | 0.000 | 0.000 | 0.000 | 0.260 | 130.568 |
| KTENVSGSPHSKAQNQQT | 3272 | 88.506 | 0.000 | 0.000 | 0.000 | 0.964 | 0.112 | 108.591 |
| KTSSGSGSPHSKAQYQQT | 3273 | 87.304 | 0.000 | 0.000 | 0.000 | 0.000 | 0.299 | 58.143 |
| KTIDGGGSPHSKAQNKQT | 3536 | 85.019 | 0.000 | 0.000 | 0.000 | 0.000 | 0.477 | 55.517 |
| KTEKVSGSPHSKAQNOQT | 3274 | 84.558 | 0.000 | 0.022 | 0.000 | 0.873 | 0.424 | 112.185 |
| KAINGSGSPHSKAQDQET | 3275 | 84.080 | 0.000 | 0.000 | 0.000 | 0.194 | 0.027 | 87.637 |
| KTCNKSGSPHSKAQNQQT | 3276 | 83.992 | 0.000 | 0.000 | 0.165 | 0.283 | 0.000 | 119.496 |
| KTINGGGSPHSKAQNQLI | 3537 | 83.881 | 0.000 | 0.000 | 0.000 | 0.046 | 0.387 | 78.383 |
| KNINGGGSPHSKAQNQQT | 3538 | 83.083 | 0.000 | 0.042 | 0.000 | 0.000 | 0.000 | 75.913 |
| KTEHLSGSPHSKAQNOQT | 3277 | 83.080 | 0.000 | 0.000 | 0.012 | 0.021 | 0.189 | 69.494 |
| KAIIGHESPHKSGONOQT | 3634 | 88.563 | 0.000 | 0.150 | 0.000 | 0.062 | 0.145 | 8.530 |
| KTINGHDSPHKTGQNQPP | 3635 | 77.357 | 0.000 | 0.149 | 0.000 | 0.000 | 0.096 | 8.865 |
| KAINGHDSPHKSGOSPQT | 68 | 75.734 | 0.095 | 0.148 | 0.000 | 0.000 | 0.238 | 14.195 |
| KAEMGSGSPHSKAQNQQT | 3278 | 83.049 | 0.000 | 0.020 | 0.000 | 0.768 | 0.112 | 135.019 |
| KATNGSGSPHSKAQNHQT | 3279 | 82.627 | 0.000 | 0.176 | 0.000 | 0.155 | 0.057 | 66.207 |
| KTIKGNDSPHKSVONQQT | 3637 | 85.986 | 0.000 | 0.135 | 0.000 | 0.263 | 0.000 | 8.603 |
| KAIKGSGSPHSKAQDOQT | 3280 | 82.258 | 0.000 | 0.000 | 0.000 | 0.108 | 0.000 | 85.178 |
| KTINGGGSPHSKSQNQLT | 3539 | 82.231 | 0.000 | 0.070 | 0.000 | 0.000 | 0.498 | 126.986 |
| KTEFGHDSPHKSGONOQT | 3638 | 77.245 | 0.000 | 0.124 | 0.000 | 0.561 | 0.063 | 16.337 |
| KTINGHDSPHKSAQNYQT | 3639 | 130.375 | 0.000 | 0.124 | 0.000 | 0.097 | 0.123 | 19.443 |
| KTFNGSASPHSKALNOQT | 3839 | 84.258 | 0.000 | 0.122 | 0.000 | 0.104 | 0.037 | 31.855 |
| KTINGCGSPHASGONOQT | 3840 | 132.540 | 0.000 | 0.121 | 0.042 | 0.000 | 0.059 | 1.857 |
| KTINAHDSPHKIGONHOT | 3640 | 106.832 | 0.000 | 0.121 | 0.000 | 0.000 | 0.231 | 5.074 |
| KTVNGNGSPHSKAQNKQT | 3540 | 81.481 | 0.000 | 0.000 | 0.000 | 0.000 | 0.122 | 69.455 |
| KTINGHESPHKSAQNRQT | 3641 | 95.531 | 0.000 | 0.113 | 0.000 | 0.130 | 0.082 | 4.815 |
| KTINGSGSPHSKGHWOQT | 3281 | 81.434 | 0.000 | 0.000 | 0.000 | 0.000 | 1.011 | 65.252 |
| KTTNGHDSPHKSGONOOG | 3642 | 85.113 | 0.000 | 0.107 | 0.000 | 0.000 | 0.017 | 10.555 |
| KTIKGQDSPHKIGQNQQT | 3643 | 110.357 | 0.000 | 0.103 | 0.058 | 0.166 | 0.135 | 11.829 |
| KTDKTSGSPHSKAQNOQT | 3282 | 81.430 | 0.000 | 0.000 | 0.000 | 1.362 | 0.291 | 169.515 |
| KTVNGHDSPHKSGQNHLT | 3644 | 81.516 | 0.000 | 0.100 | 0.017 | 0.000 | 0.028 | 16.096 |
| KTFKGSGSPHSKAPNOQT | 3283 | 80.890 | 0.000 | 0.000 | 0.000 | 0.000 | 0.017 | 71.144 |
| KSINGHDSPHKSGQYQHT | 3645 | 88.195 | 0.000 | 0.099 | 0.000 | 0.000 | 0.149 | 14.485 |
| KTINGNDSPHKSVONHOT | 3646 | 120.002 | 0.000 | 0.099 | 0.788 | 0.000 | 0.000 | 7.920 |
| KTVNGSGSPHSKAQNQLI | 3284 | 80.509 | 0.000 | 0.000 | 0.000 | 0.000 | 0.166 | 71.156 |
| KTINGSGSPHSKRPEQQT | 3285 | 80.418 | 0.000 | 0.013 | 0.000 | 0.149 | 0.361 | 50.319 |
| KTINGSGSPHSKAQRTMT | 3286 | 80.388 | 0.000 | 0.022 | 0.170 | 1.812 | 1.025 | 100.248 |
| KTITGHDSPHKSGONQWT | 3647 | 81.658 | 0.000 | 0.090 | 0.000 | 0.936 | 0.000 | 7.744 |
| KTNNGHDSPHKSVONQHT | 3648 | 115.172 | 0.000 | 0.083 | 0.000 | 0.000 | 0.062 | 7.934 |
| KTEKASGSPHSKAQNQQT | 3287 | 80.285 | 0.000 | 0.041 | 0.000 | 0.000 | 0.261 | 90.390 |
| KTIDGHDSPHKSGONQHA | 3649 | 91.058 | 0.000 | 0.082 | 0.000 | 0.000 | 0.000 | 10.781 |
| KSDQGSGSPHSKAQNOQT | 3288 | 80.076 | 0.000 | 0.000 | 0.000 | 0.993 | 0.124 | 151.911 |
| KTVNGHDSPHKSGOTROT | 3650 | 133.276 | 0.251 | 0.080 | 0.093 | 0.034 | 0.129 | 7.174 |
| KTVNGHDSPHKSGQNLHT | 3651 | 88.080 | 0.000 | 0.080 | 0.000 | 0.000 | 0.039 | 11.363 |
| KAISGHDSPHKSGLNQQT | 3652 | 78.846 | 0.000 | 0.079 | 0.000 | 0.000 | 0.015 | 11.045 |
| KTEITSGSPHSKAQNOQT | 3289 | 79.620 | 0.000 | 0.163 | 0.000 | 0.332 | 0.074 | 76.686 |
| KTDKSSGSPHSKAQNOQT | 3290 | 79.470 | 0.055 | 0.012 | 0.000 | 1.437 | 0.367 | 141.351 |
| KAINGHDSPHKSAQNQET | 3653 | 90.402 | 0.000 | 0.073 | 0.000 | 0.746 | 0.000 | 10.674 |
| KTITGHDSPHKSGQHLQT | 3654 | 137.945 | 0.000 | 0.072 | 0.000 | 0.000 | 0.000 | 4.187 |
| KTIDGSGSPHSKAQNQQH | 3291 | 79.090 | 0.000 | 0.000 | 0.000 | 0.136 | 0.049 | 57.914 |
| KTVNGNGSPHSKAQNQHT | 3541 | 78.849 | 0.000 | 0.000 | 0.000 | 0.000 | 0.045 | 54.086 |
| KNINGSGSPHSKAQNOQT | 3292 | 78.445 | 0.000 | 0.000 | 0.000 | 0.571 | 0.177 | 89.719 |
| KTINGHDSPHKSRLNQPT | 3655 | 92.883 | 0.000 | 0.070 | 0.050 | 0.904 | 1.075 | 5.598 |
| KTETHSGSPHSKAQNQQT | 3293 | 77.974 | 0.000 | 0.067 | 0.000 | 0.000 | 0.512 | 57.287 |
| KTVDGHDSPHKSGQKOOT | 3656 | 78.802 | 0.000 | 0.069 | 0.000 | 0.157 | 0.342 | 7.200 |
| KTINGQDSPHKSGQNQDT | 3657 | 82.075 | 0.000 | 0.067 | 0.000 | 0.225 | 0.144 | 9.626 |
| KTINGGGSPHSKALNQQN | 3542 | 77.822 | 0.000 | 0.131 | 0.000 | 0.000 | 0.274 | 69.884 |
| KTIEGHDSPHKSGRNOQT | 3658 | 75.838 | 0.000 | 0.065 | 0.017 | 0.000 | 0.079 | 7.818 |
| KTTNGHDSPHKSGONLLT | 3659 | 77.738 | 0.130 | 0.064 | 0.185 | 0.424 | 0.326 | 15.192 |
| KTINGHDSPHKSGOLVIT | 3660 | 76.781 | 0.089 | 0.064 | 0.000 | 0.338 | 0.475 | 11.323 |
| KTVNGHDSPHKSROSOQT | 3661 | 76.458 | 0.000 | 0.063 | 0.000 | 0.000 | 0.021 | 8.136 |
| KTINGSGSPHSKALHQHT | 3294 | 77.502 | 0.000 | 0.052 | 0.041 | 0.000 | 0.188 | 68.196 |
| KTINGHDSPHKSGRTQET | 3662 | 81.599 | 0.000 | 0.062 | 0.000 | 0.000 | 0.137 | 7.270 |
| KTINGHDSPHKSVQTHQT | 3663 | 77.309 | 0.237 | 0.062 | 0.000 | 0.000 | 0.116 | 7.519 |
| KTINGTGSPHSKAQNHQI | 3543 | 77.089 | 0.171 | 0.000 | 0.000 | 0.000 | 0.166 | 54.281 |
| KTINGSGSPHSKAQHRIT | 3295 | 76.849 | 0.105 | 0.499 | 0.170 | 1.424 | 0.214 | 127.000 |
| KTINGSGSPHSKAQYIHT | 3296 | 76.170 | 0.000 | 0.014 | 0.033 | 1.523 | 0.168 | 59.649 |
| KTSNGHDSPHKSGONOPA | 3664 | 75.834 | 0.000 | 0.056 | 0.000 | 0.000 | 0.000 | 8.501 |
| KTEGKHDSPHKSGONOQT | 3665 | 98.384 | 0.000 | 0.056 | 0.000 | 0.000 | 0.000 | 10.345 |
| KTENISGSPHSKAQNQQT | 3297 | 76.072 | 0.000 | 0.000 | 0.000 | 0.115 | 0.132 | 83.118 |
| KVINGHDSPHKSGOTOQT | 3666 | 91.665 | 0.000 | 0.055 | 1.526 | 0.311 | 0.000 | 7.391 |
| KTIIGGGSPHSKAHNOQT | 3544 | 75.872 | 0.000 | 0.050 | 0.000 | 0.000 | 0.235 | 65.492 |
| KTINGPDSPHKIGONQQS | 3667 | 85.726 | 0.000 | 0.055 | 0.171 | 0.000 | 0.063 | 10.055 |
| KTINGSGSPHSKAQKFET | 3298 | 75.788 | 0.000 | 0.000 | 0.028 | 0.108 | 0.093 | 65.588 |
| KTSNESGSPHSKAQNHQT | 3299 | 75.720 | 0.000 | 0.000 | 0.000 | 0.169 | 0.217 | 70.590 |
| KTINGSGSPHSKAQFPST | 3300 | 75.677 | 0.000 | 0.004 | 0.000 | 0.849 | 0.127 | 119.712 |
| KTERPSGSPHSKAQNOQT | 3301 | 75.669 | 0.000 | 0.029 | 0.000 | 0.000 | 0.156 | 73.894 |
| KAVNGHDSPHKSVONQQT | 3668 | 81.051 | 0.448 | 0.051 | 0.000 | 0.665 | 0.091 | 11.288 |
| KTINGNGSPHSKAQNPLT | 3545 | 75.269 | 0.000 | 0.000 | 0.000 | 0.366 | 0.000 | 53.583 |
| KSIKGNGSPHSKAQNQQT | 3546 | 75.196 | 0.000 | 0.000 | 0.000 | 0.000 | 0.000 | 90.251 |
| KTINGHDSPHKSRQDQHT | 3669 | 75.595 | 0.000 | 0.049 | 0.118 | 0.030 | 0.045 | 8.540 |
| KAINGPDSPHKSGQKOQT | 3670 | 78.213 | 0.464 | 0.047 | 0.000 | 0.323 | 0.162 | 10.395 |
| KTINGHDSPHKSRQSQHT | 3671 | 88.544 | 0.499 | 0.046 | 0.000 | 0.059 | 0.032 | 8.324 |
| KTIYGHDSPHKSVONQLT | 3672 | 92.381 | 0.000 | 0.043 | 0.000 | 0.103 | 0.016 | 12.323 |
| KTVNGHDSPHKSGONLLT | 3673 | 83.969 | 0.114 | 0.040 | 0.023 | 0.000 | 0.035 | 18.894 |
| KTESAHDSPHKSGONOQT | 3674 | 80.810 | 0.000 | 0.039 | 0.000 | 0.000 | 0.000 | 13.338 |
| KTENKSGSPHSKAQNOQT | 3594 | 103.854 | 0.000 | 0.037 | 0.000 | 0.000 | 0.119 | 31.182 |
| KTTNGQDSPHKSGONQQS | 3675 | 92.419 | 0.000 | 0.037 | 0.043 | 0.000 | 0.079 | 7.592 |
| KTDKGSGSPHSKAQNQQT | 3595 | 94.572 | 0.000 | 0.037 | 0.000 | 0.951 | 0.367 | 47.888 |
| KTIDGHDSPHKSGRNQQI | 3676 | 80.240 | 0.000 | 0.037 | 0.000 | 0.040 | 0.144 | 10.363 |
| KTINGYDSPHKSGQYQHT | 3677 | 81.534 | 0.000 | 0.036 | 0.000 | 0.000 | 0.000 | 10.524 |
| KTDNGHDSPHKSRONOQT | 3678 | 105.312 | 0.000 | 0.033 | 0.000 | 0.000 | 0.018 | 7.931 |
| KTINGHDSPHKSWVROQT | 3679 | 125.537 | 0.000 | 0.033 | 0.000 | 0.291 | 0.174 | 11.687 |
| KTINGHESPHKSGONQHS | 3680 | 92.248 | 0.000 | 0.032 | 0.012 | 0.090 | 0.088 | 9.720 |
| KTVNGHDSPHKIGHNQQT | 3681 | 120.985 | 0.000 | 0.029 | 0.000 | 0.000 | 0.009 | 10.167 |
| KTCNGHDSPHKSGRNOQT | 3682 | 94.616 | 0.000 | 0.025 | 0.000 | 0.000 | 0.128 | 12.496 |
| KTINGNGSPHSKAQNHQA | 3841 | 88.274 | 0.000 | 0.024 | 0.000 | 0.000 | 0.041 | 36.754 |
| KNVVGHDSPHKSGONOQT | 3683 | 75.330 | 0.000 | 0.024 | 0.000 | 0.063 | 0.049 | 8.077 |
| KTELWHDSPHKSGONOQT | 3684 | 85.323 | 0.057 | 0.020 | 0.000 | 0.000 | 0.243 | 9.915 |
| KTELRHDSPHKSGONOQT | 3685 | 98.098 | 0.000 | 0.019 | 0.000 | 0.000 | 0.007 | 6.588 |
| KTINGHDSPHKSNAWOQT | 3686 | 84.825 | 0.000 | 0.016 | 0.000 | 0.000 | 0.132 | 15.788 |
| KTDAGHDSPHKSGONOQT | 3687 | 88.924 | 0.000 | 0.013 | 0.000 | 1.076 | 0.070 | 18.107 |
| KTEVGHDSPHKSGONOQT | 3688 | 112.457 | 0.000 | 0.011 | 0.000 | 0.000 | 0.138 | 13.125 |
| KTESRHDSPHKSGONOQT | 3689 | 81.766 | 0.000 | 0.011 | 0.000 | 0.052 | 0.036 | 6.975 |
| KSELGHDSPHKSGONOQT | 3690 | 107.059 | 0.000 | 0.005 | 0.000 | 0.000 | 0.055 | 13.285 |
| KTINGHDSPHKSGQSVPT | 3691 | 77.840 | 0.000 | 0.003 | 0.000 | 0.136 | 0.061 | 6.768 |
| KTINGHESPHKSGONIQP | 3692 | 253.840 | 0.000 | 0.000 | 0.000 | 0.000 | 0.000 | 14.042 |
| KTEMKHDSPHKSGONQQT | 3693 | 240.075 | 0.000 | 0.000 | 0.000 | 0.000 | 0.000 | 3.183 |
| KTINGHDSPHKSVONHLN | 3694 | 196.758 | 0.000 | 0.000 | 0.000 | 0.000 | 0.000 | 14.557 |
| KTINGHDSPHKIGLDOQT | 3695 | 165.627 | 0.000 | 0.000 | 0.000 | 1.942 | 0.000 | 5.469 |
| KTSNASGSPHSKAQHQQT | 3596 | 165.206 | 0.000 | 0.000 | 0.000 | 0.000 | 0.082 | 40.558 |
| KTINGHDSPHKRGPDQQS | 3696 | 160.084 | 0.000 | 0.000 | 0.000 | 0.000 | 0.000 | 2.923 |
| KTINGMGSPHSKTQNOQT | 3842 | 158.728 | 0.000 | 0.000 | 0.000 | 0.000 | 0.638 | 47.809 |
| KTIKGHDSPHKSGESQQT | 3697 | 142.264 | 0.000 | 0.000 | 0.000 | 0.000 | 0.218 | 4.176 |
| KTEGWHDSPHKSGONOQT | 3698 | 142.064 | 0.000 | 0.000 | 0.000 | 0.000 | 0.264 | 11.785 |
| KTINGHDSPHKHGQNHQT | 3699 | 141.405 | 0.191 | 0.000 | 0.000 | 0.000 | 0.000 | 10.214 |
| KTEQLHDSPHKSGONOQT | 3700 | 138.345 | 0.000 | 0.000 | 0.000 | 0.000 | 0.000 | 12.606 |
| KTVNGTGSPHSKAQNQLT | 3843 | 137.639 | 0.000 | 0.000 | 0.000 | 0.000 | 0.277 | 48.950 |
| KTIIGHDSPHKSGQYQHT | 3701 | 131.825 | 0.000 | 0.000 | 0.000 | 0.000 | 0.211 | 5.762 |
| KTSNGHDSPHKSVONKQT | 3702 | 130.640 | 0.000 | 0.000 | 0.000 | 0.172 | 0.039 | 11.850 |
| KIVNGQVSPHKSGONOQT | 3703 | 129.649 | 0.000 | 0.000 | 0.000 | 0.000 | 0.031 | 16.942 |
| KTVNGHDSPHKSGORQLT | 3704 | 129.641 | 0.000 | 0.000 | 0.000 | 0.000 | 0.487 | 20.145 |
| KTVNGHDSPHKIGQNQLT | 3705 | 128.582 | 0.000 | 0.000 | 0.499 | 0.027 | 0.199 | 20.957 |
| KTINGHDSPHKSGQIIVT | 3706 | 125.245 | 0.000 | 0.000 | 0.151 | 0.000 | 0.379 | 6.808 |
| KTEKIHDSPHKSGONOQT | 3707 | 125.178 | 0.000 | 0.000 | 0.000 | 0.000 | 0.000 | 17.604 |
| KTENAHDSPHKSGONOQT | 3708 | 124.477 | 0.000 | 0.000 | 0.000 | 0.000 | 0.062 | 15.805 |
| KIGNGHESPHKSGONOQT | 3709 | 123.324 | 0.000 | 0.000 | 0.000 | 0.000 | 0.000 | 11.198 |
| KEVMGHDSPHKSGONOQT | 3710 | 121.107 | 0.000 | 0.000 | 0.000 | 0.000 | 0.000 | 17.191 |
| KTEVKHDSPHKSGONOQT | 3711 | 119.733 | 0.000 | 0.000 | 0.000 | 0.000 | 0.000 | 5.550 |
| KTINGYDSPHKSGQKOST | 3712 | 119.615 | 0.000 | 0.000 | 0.000 | 0.000 | 0.000 | 7.970 |
| KTIHGNGSPHSKAQNQET | 3844 | 117.388 | 0.000 | 0.000 | 0.000 | 0.000 | 0.000 | 38.874 |
| KYQVGHDSPHKSGONOQT | 3713 | 112.797 | 0.000 | 0.000 | 0.000 | 0.000 | 0.542 | 9.335 |
| KTEAMHDSPHKSGONOQT | 3714 | 111.765 | 0.000 | 0.000 | 0.000 | 0.000 | 0.000 | 16.142 |
| KTIKGDDSPHKSVONQQT | 3715 | 109.397 | 0.000 | 0.000 | 0.000 | 0.000 | 0.000 | 19.125 |
| KTINGHDSPHKSVQSHOT | 3716 | 109.375 | 0.107 | 0.000 | 0.319 | 0.000 | 0.547 | 12.617 |
| KTINGHDSPHKSGQFVVT | 3717 | 108.725 | 0.000 | 0.000 | 0.000 | 0.124 | 0.406 | 10.179 |
| KTVNGHDSPHKSRQNLQT | 3718 | 107.496 | 0.205 | 0.000 | 0.000 | 1.934 | 0.062 | 8.616 |
| KATNGHNSPHKSGONQET | 3719 | 106.806 | 0.000 | 0.000 | 0.000 | 0.000 | 0.000 | 10.566 |
| KAINGHDSPHKSAQNQQI | 3720 | 106.539 | 0.000 | 0.000 | 0.000 | 0.000 | 0.113 | 21.786 |
| KTEHGHDSPHKSGONOQT | 3721 | 106.486 | 0.000 | 0.000 | 0.000 | 0.000 | 0.006 | 14.956 |
| KTVENHDSPHKSGONQQT | 3722 | 106.468 | 0.000 | 0.000 | 0.000 | 0.000 | 0.156 | 9.246 |
| KTIYGHDSPHKSGQSQPT | 3723 | 106.431 | 0.000 | 0.000 | 0.000 | 0.155 | 0.137 | 6.562 |
| KTISGHESPHKSGONEQT | 3724 | 105.740 | 0.000 | 0.000 | 0.000 | 0.378 | 1.384 | 9.156 |
| KAIIGHDSPHKSAQNOOT | 3725 | 105.292 | 0.000 | 0.000 | 0.000 | 0.000 | 0.553 | 16.793 |
| KAIDGHDSPHKSGQNQLT | 3726 | 104.701 | 0.331 | 0.000 | 0.000 | 0.201 | 0.638 | 16.109 |
| KTIMGHDSPHKSVONQQT | 3727 | 104.683 | 0.000 | 0.000 | 0.000 | 0.000 | 0.000 | 8.029 |
| KEVGGHDSPHKSGONOQT | 3728 | 103.896 | 0.000 | 0.000 | 0.000 | 0.000 | 0.000 | 16.899 |
| KTINGHDSPHKSAQNLLT | 3729 | 103.332 | 0.000 | 0.000 | 0.256 | 0.194 | 0.000 | 15.722 |
| KTEFTHDSPHKSGONOQT | 3730 | 102.052 | 0.062 | 0.000 | 0.000 | 0.439 | 0.047 | 12.527 |
| KTINASGSPHSKAINQQT | 3597 | 101.122 | 0.233 | 0.000 | 0.000 | 0.000 | 0.145 | 47.196 |
| KAINGNGSPHKRGONOQT | 3845 | 100.925 | 0.000 | 0.000 | 0.000 | 0.000 | 0.159 | 10.011 |
| KSEMGHDSPHKSGONOQT | 3731 | 100.539 | 0.000 | 0.000 | 0.000 | 0.000 | 0.000 | 18.356 |
| KAQQGHDSPHKSGONOQT | 3732 | 100.395 | 0.000 | 0.000 | 0.000 | 0.000 | 0.057 | 3.954 |
| KTEVMHDSPHKSGQNQQT | 3733 | 99.473 | 0.000 | 0.000 | 0.000 | 0.000 | 0.000 | 12.400 |
| KAINGHDSPHKSGQSLOT | 3734 | 99.310 | 0.058 | 0.000 | 1.439 | 0.254 | 0.056 | 17.323 |
| KTINGSGSPHSKAPNQQH | 3598 | 99.300 | 0.252 | 0.000 | 0.000 | 0.000 | 0.038 | 39.297 |
| KCGEGHDSPHKSGONOQT | 3735 | 99.298 | 0.000 | 0.000 | 0.000 | 0.000 | 0.000 | 13.147 |
| KTVNGHDSPHKSAQNHQT | 3736 | 99.257 | 0.000 | 0.000 | 0.078 | 0.000 | 0.027 | 17.639 |
| KTVNGHDSPHKSGOTQLT | 3737 | 98.524 | 0.000 | 0.000 | 0.313 | 0.183 | 0.172 | 14.883 |
| KTNNGHDSPHKSGRNRQT | 3738 | 98.307 | 0.000 | 0.000 | 0.124 | 0.000 | 0.037 | 5.840 |
| KTCNEHDSPHKSGONOQT | 3739 | 97.092 | 0.000 | 0.000 | 0.000 | 0.000 | 0.000 | 8.364 |
| KTINGHDSPHKYGONEQT | 3740 | 96.960 | 0.000 | 0.000 | 0.000 | 0.000 | 0.000 | 4.613 |
| KASNRHDSPHKSGHNOQT | 3741 | 96.283 | 0.000 | 0.000 | 0.000 | 0.000 | 0.675 | 8.045 |
| KTINGNGSPHSKAPNLQT | 3846 | 95.963 | 0.000 | 0.000 | 0.000 | 0.000 | 0.247 | 36.341 |
| KTETKHDSPHKSGONOQT | 3742 | 95.121 | 0.000 | 0.000 | 0.000 | 0.000 | 0.083 | 4.831 |
| KSINGHDSPHKSQQNOQT | 3743 | 94.479 | 0.000 | 0.000 | 0.000 | 0.000 | 1.696 | 9.633 |
| KTIGGHDSPHKSGONQQI | 3744 | 94.420 | 0.000 | 0.000 | 0.000 | 0.000 | 0.333 | 19.324 |
| KTDPQHDSPHKSGONOQT | 3745 | 93.931 | 0.000 | 0.000 | 0.000 | 0.906 | 0.019 | 11.749 |
| KTINRHDSPHKIVONQQT | 3746 | 93.409 | 0.000 | 0.000 | 0.000 | 0.000 | 0.000 | 3.064 |
| KTEQYHDSPHKSGONOQT | 3747 | 93.065 | 0.000 | 0.000 | 0.000 | 0.000 | 0.047 | 15.190 |
| KTINGHDSPHKSVQSKOT | 3748 | 92.445 | 0.000 | 0.000 | 0.078 | 0.000 | 0.047 | 4.263 |
| KELVGHDSPHKSGONOQT | 3749 | 92.262 | 0.000 | 0.000 | 0.000 | 0.594 | 0.000 | 14.890 |
| KTENRHDSPHKSGONOQT | 3750 | 91.675 | 0.000 | 0.000 | 0.000 | 0.000 | 0.000 | 13.282 |
| KELMGHDSPHKSGONOQT | 3751 | 91.191 | 0.000 | 0.000 | 0.000 | 0.000 | 0.000 | 14.032 |
| KTINGNDSPHKIGHNOQT | 3752 | 91.183 | 0.000 | 0.000 | 0.117 | 0.000 | 0.270 | 11.095 |
| KTIKGGGSPHSKAQDQQT | 3847 | 91.172 | 0.000 | 0.000 | 0.000 | 0.064 | 0.085 | 49.580 |
| KTEGHHDSPHKSGONOQT | 3753 | 89.922 | 0.000 | 0.000 | 0.000 | 0.000 | 0.000 | 23.929 |
| KTEGYHDSPHKSGONOQT | 3754 | 89.891 | 0.000 | 0.000 | 0.000 | 0.000 | 0.000 | 15.116 |
| KTVNGHDSPHKSGOTQQI | 3755 | 89.801 | 0.000 | 0.000 | 0.657 | 0.000 | 0.542 | 11.543 |
| KTINGQDSPHKSGONPLT | 3756 | 89.726 | 0.000 | 0.000 | 0.000 | 0.363 | 0.000 | 15.561 |
| KTVNASGSPHSKAQNHQT | 3599 | 89.467 | 0.091 | 0.000 | 0.000 | 0.045 | 0.252 | 39.024 |
| KTINGHDSPHKSGRDQKT | 3757 | 88.871 | 0.000 | 0.000 | 0.000 | 0.350 | 0.181 | 12.117 |
| KTINGHDSPHKSVHNQQN | 3758 | 88.715 | 0.089 | 0.000 | 0.081 | 0.000 | 0.143 | 10.787 |
| KTINGHDSPHKSGQWKRT | 3759 | 88.633 | 0.000 | 0.000 | 0.000 | 0.202 | 0.094 | 5.186 |
| KTIDGSGSPHSKAENRQT | 3600 | 87.993 | 0.092 | 0.000 | 0.000 | 0.139 | 0.054 | 40.629 |
| KNEIGHDSPHKSGONOQT | 3760 | 87.758 | 0.000 | 0.000 | 0.000 | 0.000 | 0.055 | 14.110 |
| KAINGHDSPHKSGOSQQI | 3761 | 87.585 | 0.000 | 0.000 | 5.310 | 0.000 | 0.000 | 12.864 |
| KIINGHDSPHKSRQAQQT | 3762 | 86.966 | 0.000 | 0.000 | 0.000 | 0.000 | 0.000 | 9.193 |
| KTPNGHDSPHKSGONQQI | 3763 | 86.683 | 0.000 | 0.000 | 0.000 | 0.000 | 0.109 | 21.278 |
| KITNGHDSPHKSGOTOQT | 3764 | 86.443 | 0.000 | 0.000 | 0.000 | 0.192 | 0.190 | 17.479 |
| KTINGHDSPHKSVONHQI | 3765 | 86.395 | 0.000 | 0.000 | 0.000 | 0.000 | 0.000 | 9.148 |
| KTINGHDSPHKSKONQQA | 3766 | 86.265 | 0.000 | 0.000 | 0.000 | 0.123 | 0.041 | 5.768 |
| KTINGHDSPHKSAQNQLN | 3767 | 86.153 | 0.000 | 0.000 | 0.000 | 0.050 | 0.019 | 15.587 |
| KTDITHDSPHKSGONOQT | 3768 | 85.876 | 0.000 | 0.000 | 0.000 | 0.000 | 0.013 | 9.076 |
| KTVNGHDSPHKSGOTQPT | 3769 | 85.680 | 0.000 | 0.000 | 1.301 | 1.064 | 0.000 | 8.067 |
| KTEKFHDSPHKSGONOQT | 3770 | 85.358 | 0.000 | 0.000 | 0.000 | 0.000 | 0.026 | 7.229 |
| KTDQGHDSPHKSGONOQT | 3771 | 85.267 | 0.000 | 0.000 | 0.000 | 0.000 | 0.000 | 16.042 |
| KTINGHDSPHKLWINQQT | 3772 | 85.132 | 0.000 | 0.000 | 1.154 | 0.000 | 0.017 | 12.704 |
| KGINGPDSPHKSGONQQT | 3773 | 85.080 | 0.000 | 0.000 | 0.084 | 0.000 | 0.054 | 13.750 |
| KSEIGHDSPHKSGQNQQT | 3774 | 84.789 | 0.000 | 0.000 | 0.000 | 0.000 | 0.013 | 15.955 |
| KTINGHDSPHKSVQKQLT | 3775 | 84.351 | 0.000 | 0.000 | 0.000 | 0.038 | 0.103 | 11.890 |
| KTINGHPSPHWKGONOQT | 3848 | 84.153 | 0.000 | 0.000 | 0.000 | 0.000 | 0.058 | 3.280 |
| KTVNGHDSPHKSGRNQLA | 3776 | 83.858 | 0.000 | 0.000 | 0.000 | 0.000 | 0.132 | 21.252 |
| KTNNVHDSPHKSGONQQS | 3777 | 83.697 | 0.000 | 0.000 | 0.000 | 0.176 | 0.000 | 7.117 |
| KTIKGSGSPHSKVODOQT | 3601 | 83.077 | 0.000 | 0.000 | 0.034 | 0.000 | 0.107 | 21.001 |
| KSEKGHDSPHKSGQNQQT | 3778 | 82.982 | 0.000 | 0.000 | 0.000 | 0.000 | 0.105 | 16.662 |
| KWSAGHDSPHKSGONOQT | 3779 | 82.949 | 0.000 | 0.000 | 0.000 | 0.000 | 0.211 | 12.499 |
| KELAGHDSPHKSGONOQT | 3780 | 82.876 | 0.000 | 0.000 | 0.000 | 0.000 | 0.093 | 18.063 |
| KTINGHDSPHKMGRNQQS | 3781 | 82.787 | 0.000 | 0.000 | 0.000 | 0.000 | 0.000 | 6.467 |
| KTDQAHDSPHKSGONOQT | 3782 | 82.402 | 0.000 | 0.000 | 0.141 | 0.000 | 0.000 | 13.397 |
| KTETQHDSPHKSGONOQT | 3783 | 82.316 | 0.000 | 0.000 | 0.000 | 0.000 | 0.198 | 10.823 |
| KTEMTHDSPHKSGONOQT | 3784 | 82.221 | 0.000 | 0.000 | 0.000 | 0.000 | 0.000 | 8.431 |
| KTINGHDSPHKSGISIQT | 3785 | 82.019 | 0.000 | 0.000 | 0.000 | 0.191 | 0.044 | 7.310 |
| KTDAVHDSPHKSGONOQT | 3786 | 81.968 | 0.000 | 0.000 | 0.000 | 0.297 | 0.107 | 13.596 |
| KTSNGHDSPHKSVQNLOT | 3787 | 81.921 | 0.000 | 0.000 | 0.072 | 0.000 | 0.330 | 11.544 |
| KTEKYHDSPHKSGONOQT | 3788 | 81.637 | 0.000 | 0.000 | 0.000 | 0.000 | 0.013 | 7.580 |
| KOTQGHDSPHKSGONOQT | 3789 | 81.581 | 0.000 | 0.000 | 0.000 | 0.000 | 0.133 | 15.225 |
| KTINGHDSPHKMAHNQQT | 3790 | 81.329 | 0.000 | 0.000 | 0.000 | 0.000 | 0.094 | 15.949 |
| KAINGSGSPHSKAQTQQA | 3602 | 81.207 | 0.000 | 0.000 | 0.000 | 0.000 | 0.016 | 40.435 |
| KTINGHDSPHKHGONQQN | 3791 | 81.065 | 0.000 | 0.000 | 0.000 | 0.000 | 0.000 | 4.110 |
| KGADGHDSPHKSGONOQT | 3792 | 80.981 | 0.000 | 0.000 | 0.000 | 0.000 | 0.074 | 11.423 |
| KVGEGHDSPHKSGONOQT | 3793 | 80.775 | 0.084 | 0.000 | 0.000 | 0.000 | 0.019 | 16.378 |
| KANEGHDSPHKSGONQQT | 3794 | 80.470 | 0.000 | 0.000 | 0.000 | 0.000 | 0.000 | 12.818 |
| KTDTMHDSPHKSGONOQT | 3795 | 80.364 | 0.000 | 0.000 | 0.000 | 0.000 | 0.000 | 13.166 |
| KTEAKSGSPHSKAQNQQT | 3603 | 80.088 | 0.192 | 0.000 | 0.000 | 0.000 | 0.613 | 47.130 |
| KTINGHDSPHKSVOSQQS | 3796 | 80.000 | 0.000 | 0.000 | 0.000 | 1.055 | 0.082 | 17.620 |
| KTIPGSGSPHSKAQNLQT | 3604 | 79.973 | 0.871 | 0.000 | 0.000 | 0.000 | 0.000 | 32.693 |
| KTCIAHDSPHKSGONOQT | 3797 | 79.857 | 0.000 | 0.000 | 0.066 | 0.000 | 0.093 | 1.930 |
| KTINGHDSPHKSGQTVCT | 3798 | 79.730 | 0.000 | 0.000 | 0.000 | 0.050 | 0.030 | 7.873 |
| KELRGHDSPHKSGONOQT | 3799 | 79.596 | 0.000 | 0.000 | 0.000 | 0.000 | 0.006 | 22.001 |
| KCQIGHDSPHKSGONOQT | 3800 | 79.359 | 0.000 | 0.000 | 0.000 | 0.000 | 0.000 | 2.614 |
| KGVMGHDSPHKSGONOQT | 3801 | 79.170 | 0.000 | 0.000 | 0.000 | 0.138 | 0.086 | 17.287 |
| KACDGHDSPHKSGONOQT | 3802 | 78.648 | 0.000 | 0.000 | 0.000 | 0.000 | 0.128 | 17.767 |
| KTINGQDSPHKSGOYQQI | 3803 | 78.585 | 0.000 | 0.000 | 0.000 | 0.286 | 0.672 | 5.664 |
| KTINGHDSPHKSGQQIMT | 3804 | 78.534 | 0.000 | 0.000 | 0.000 | 0.000 | 0.058 | 7.067 |
| KTINGHDSPHKSRONEQS | 3805 | 78.534 | 0.000 | 0.000 | 0.000 | 0.112 | 0.188 | 13.388 |
| KASNGHDSPHKSGLNHQT | 3806 | 78.451 | 0.000 | 0.000 | 0.000 | 0.000 | 0.000 | 17.975 |
| KTVNGHDSPHKSGOSQPT | 3807 | 78.309 | 0.000 | 0.000 | 0.000 | 0.000 | 0.231 | 10.627 |
| KNELGHDSPHKSGONOQT | 3808 | 78.135 | 0.000 | 0.000 | 0.000 | 0.000 | 0.182 | 17.457 |
| KTETFHDSPHKSGONOQT | 3809 | 78.070 | 0.000 | 0.000 | 0.000 | 0.782 | 0.007 | 4.693 |
| KAAEGHDSPHKSGONOQT | 3810 | 77.793 | 0.000 | 0.000 | 0.000 | 0.000 | 0.060 | 13.552 |
| KGQNGHDSPHKSGONOQT | 3811 | 77.770 | 0.000 | 0.000 | 0.000 | 0.107 | 0.056 | 13.618 |
| KNEFGHDSPHKSGONOQT | 3812 | 77.740 | 0.000 | 0.000 | 0.000 | 0.000 | 0.029 | 16.318 |
| KTSIGYDSPHKSGONOQT | 3813 | 77.730 | 0.000 | 0.000 | 0.000 | 0.057 | 0.178 | 4.831 |
| KTDNGHDSPHKSGONLQT | 3814 | 77.565 | 0.504 | 0.000 | 0.000 | 0.000 | 0.000 | 16.184 |
| KTEGQHDSPHKSGONOQT | 3815 | 77.423 | 0.000 | 0.000 | 0.000 | 0.000 | 0.748 | 20.310 |
| KTITGHDSPHKSRODOQT | 3816 | 77.127 | 0.000 | 0.000 | 0.000 | 0.000 | 0.000 | 6.250 |
| KAEHGHDSPHKSGONOQT | 3817 | 77.026 | 0.000 | 0.000 | 0.000 | 0.000 | 0.017 | 20.937 |
| KTINGDDSPHKSGQKOLT | 3818 | 76.968 | 0.000 | 0.000 | 0.000 | 0.163 | 0.014 | 15.820 |
| KCDQGHDSPHKSGONQQT | 3819 | 76.887 | 0.000 | 0.000 | 0.000 | 0.193 | 0.013 | 27.317 |
| KEILGHDSPHKSGONOQT | 3820 | 76.770 | 0.000 | 0.000 | 0.000 | 0.804 | 0.009 | 10.771 |
| KTIHGSGSPHSKAQNQAT | 3605 | 76.765 | 0.000 | 0.000 | 0.000 | 0.000 | 0.215 | 43.969 |
| KTERNHDSPHKSGONOQT | 3821 | 76.751 | 0.000 | 0.000 | 0.000 | 0.000 | 0.000 | 14.979 |
| KAINGDDSPHKSGHNQQT | 3822 | 76.578 | 0.000 | 0.000 | 0.000 | 0.032 | 0.059 | 17.755 |
| KTSNGHNSPHKSGONQET | 3823 | 76.515 | 0.000 | 0.000 | 0.000 | 0.000 | 0.000 | 4.764 |
| KTINGHDSPHKSGQMIHT | 3824 | 76.364 | 0.000 | 0.000 | 0.000 | 0.000 | 0.000 | 9.486 |
| KNAIGHDSPHKSGONOQT | 3825 | 76.289 | 0.000 | 0.000 | 0.000 | 0.009 | 0.072 | 15.178 |
| KTDKFHDSPHKSGONOQT | 3826 | 76.204 | 0.000 | 0.000 | 0.000 | 0.000 | 0.000 | 7.096 |
| KTEGFHDSPHKSGONOQT | 3827 | 76.191 | 0.000 | 0.000 | 0.000 | 0.000 | 0.080 | 13.163 |
| KVINGHDSPHKSGRNHQS | 3828 | 75.961 | 0.000 | 0.000 | 0.000 | 0.000 | 0.000 | 13.568 |
| KTITGHDSPHKSVONRQT | 3829 | 75.940 | 0.000 | 0.000 | 0.000 | 0.621 | 0.000 | 4.310 |
| KTPDMHDSPHKSGONOQT | 3830 | 75.871 | 0.659 | 0.000 | 0.000 | 0.000 | 0.048 | 11.277 |
| KTINGHDSPHKSGQKMNT | 3831 | 75.820 | 0.000 | 0.000 | 0.000 | 0.000 | 0.167 | 6.373 |
| KTELQHDSPHKSGONOQT | 3832 | 75.814 | 0.000 | 0.000 | 0.000 | 0.105 | 0.000 | 11.798 |
| KTIHGHDSPHKSGOSQQN | 3833 | 75.777 | 0.000 | 0.000 | 0.059 | 0.000 | 0.166 | 7.426 |
| KTEIGHDSPHKSGONOQT | 3834 | 75.525 | 0.000 | 0.000 | 0.016 | 0.012 | 0.000 | 9.593 |
| KTINGHDSPHKSGOYQHA | 3835 | 75.308 | 0.000 | 0.000 | 0.000 | 0.000 | 0.017 | 17.081 |
| KTELYHDSPHKSGONOQT | 3836 | 75.235 | 0.000 | 0.000 | 0.000 | 0.000 | 0.042 | 10.354 |

Additional variants were identified following generation and screening in NHPs that had the following properties. TTM-001 and TTM-002 capsid variants comprising the amino acid sequence of SEQ ID NOs: 4253, 4281, 4290-4295, 4304, 4305, 4320, 4328-4335, 4337-4340, 4353, 4355, 4369, 4387, 4421, 4424, 4425, 4426, 4427, 4428, 4430, 4432, 4433, 4435, 4436, 4437, 4438, 4439-4449, 4452, 4455, 4476, 4483, or 4484 had a raw virus count 10 or greater, a CV of less than 1 for the brain samples isolated from the NHPs, demonstrated a 50-fold or greater increase in expression in the brain of mice and NHPs relative to AAV9, and demonstrated 2-fold or less expression in the liver and DRG of NHPs relative to AAV9. TTM-001 and TTM-002 capsid variants comprising the amino acid sequence of SEQ ID NOs: 4098-4105, 4254-4280, 4282-4289, 4296-4303, 4306-4327, 4336, 4341-4352, 4354, 4356-4361, 4362-4420, 4422, 4423, 4425, 4429, 4431, 4434, 4444, 4450, 4451, 4453, 4454, 4456-4471, 4472, 4473, 4474, 4475, 4477, 4478, 4479, 4480, 4481, 4482, or 4485 had a CV of less than 1 in across the brain samples isolated from the NHPs and demonstrated a 100-fold or greater increase in expression in the brain of NHPs relative to AAV9. TTM-001 and TTM-002 capsid variants comprising the amino acid sequence of SEQ ID NOs: 4102 and 4106-4252 had normalized virus counts of greater than or equal to 0.01, a CV of less than 1 across the liver RNA samples isolated from the NHPs, and demonstrated a 20-fold or greater increase in expression in the liver of NHPs relative to AAV9. TM-001 and TTM-002 capsid variants comprising the amino acid sequence of SEQ ID NO: 4105 had a raw virus count 9.9 or greater, a CV of less than 1 across the muscle samples isolated from the NHPs, and 5-fold or greater increase in expression in the muscle of the NHPs relative to AAV9. TM-001 and TTM-002 capsid variants comprising the amino acid sequence of SEQ ID NO: 4105 also had a raw virus count 9.9 or greater, a CV of less than 1 across the samples isolated from the heart of the NHPs, and 5-fold or greater increase in expression in the heart of the NHPs relative to AAV9.

These additional matured variants were then incorporated into a synthetic library that was passaged through another NHP or mouse along with the TTM-002 capsid variant, the TTM-001 capsid variant, and an AAV9 capsid as controls. Following this passage, CNS and peripheral tissues were collected and RNA was extracted. Following RNA recovery and RT-PCR amplification, a systematic NGS enrichment analysis was performed to calculate the fold enrichment ratio relative to the AAV9 wild-type control or the TTM-002 capsid variant control. The matured capsid variants were filtered first by capsid fitness (virus counts/DNA counts), selecting those samples that had a fitness threshold greater than or equal to 0.75. Following this initial selection, brain tropic and heart tropic matured capsid variants were selected. For brain tropic capsid variants with little to no expression in the liver (liver de-targeted) or heart, the matured capsid variants were further filtered based on a CV of less than 0.5 across the brain RNA samples isolated from the NHPs, a fold-change in DNA expression in the liver of the NHPs of less than 0.2 relative to the AAV9 wild-type control, a ratio of brain RNA to liver DNA in NHPs of at least 100, at least 2.5 or 3 fold-enrichment or greater relative to the TTM-002 control in the brain of the NHPs, at least 0.8 fold-enrichment or greater in the brain of mice relative to the TTM-002 control, and an average fold-change in expression of RNA in the heart relative to the AAV9 control of less than 1.5. The resulting brain tropic matured capsid variants that lead to increased expression in the brain of mice and NHPs comprised the amino acid sequences of SEQ ID NOs: 3272, 3274, 3421, 3487, 3589, and 4478, many of which comprised an E at position 451 numbered according to SEQ ID NOs: 69, 70, 71, 4, 5, 6, 7, 8, 36-59, 138, 981, or 982 (or the third position numbered according to SEQ ID NOs: 3272, 3274, 3421, 3487, 3589, and 4478), and either an N or a positively charged residue (e.g., K or R) at position 452 numbered according to SEQ ID NO: 69, 70, 71, 4, 5, 6, 7, 8, 36-59, 138, 981, or 982 (or the fourth position numbered according to SEQ ID NOs: 3272, 3274, 3421, 3487, 3589, and 4478). For heart tropic capsids, the capsid variants were further filtered based on a CV of less than 0.5 across the heart RNA samples isolated from the NHPs, a fold-change in DNA expression in the liver of the NHPs of less than 0.5 relative to the AAV9 wild-type control, and a fold-change in RNA expression of less than 1.5 in the brain of NHPs relative to the TTM-002 control. The resulting heart tropic matured capsid variants comprised the amino sequence of SEQ ID NOs: 3382, 4081, and 4406. The data and sequences for the selected brain and heart tropic capsid variants that met the filtering criteria are provided in Table 30. These brain and heart tropic matured capsid variants also demonstrated decreased tropism in the DRG.

TABLE 30
NGS fold-enrichment of TTM-001 and TTM-002 matured AAV capsid variants with
increased expression in the brain or heart of NHPs and mice

| | | Average | | | | | | |
|---|---|---|---|---|---|---|---|---|
| | | Fold | | | | | | |
| | | Enrichment | | | | | | |
| | | relative to | | | | | | |
| | | TTM-002 | Average Fold Enrichment relative to AAV9 | | | | | |
| | | Brain RNA | Brain | Spinal | Liver | Heart | Muscle | Brain |
| | SEQ | (NHP) | RNA | Cord | DNA | RNA | DNA | RNA |
| Sequence | ID NO | | (NHP) | (NHP) | (NHP) | (NHP) | (NHP) | (Mouse) |
| KTENVSGSPHSKAQNQQT | 3272 | 7.333 | 35.140 | 12.982 | 0.088 | 1.242 | 2.077 | 8.619 |
| KTEKVSGSPHSKAQNQQT | 3274 | 6.203 | 29.725 | 3.103 | 0.138 | 1.165 | 0.855 | 6.073 |
| KTERVSGSPHSKAQNQQT | 3589 | 4.147 | 19.870 | 4.171 | 0.123 | 0.872 | 5.141 | 6.080 |
| KNSIGSGSPHSKAQNQQT | 3421 | 3.582 | 17.164 | 3.080 | 0.137 | 0.798 | 0.772 | 7.657 |
| KTVNGHDSPHKSGQRPST | 4478 | 2.890 | 13.848 | 5.763 | 0.110 | 0.551 | 4.544 | 6.833 |
| KAENGSGSPHSKAQNQQT | 3487 | 2.627 | 12.588 | 3.183 | 0.079 | 0.526 | 1.945 | 7.571 |
| KTSNASGSPHSKAHNQQT | 3382 | 1.419 | 6.799 | 8.066 | 0.352 | 6.641 | 7.517 | 8.203 |
| KTINGSGSPHKSGQNQQP | 4081 | 0.784 | 3.759 | 1.946 | 0.206 | 4.799 | 21.127 | 5.882 |
| KTINGSGSPHKSGQNKTS | 4406 | 0.550 | 2.637 | 0.655 | 0.317 | 7.899 | 12.738 | 1.932 |
| KTINGSGSPHSKAQNQQT | 5660 | 1.297 | 6.216 | 5.338 | 0.456 | 3.062 | 4.732 | 5.964 |
| KTINGHDSPHKSGQNQQT | 5828 | 1.000 | 4.792 | 2.504 | 0.181 | 2.456 | 4.591 | 7.103 |

These data demonstrate that following two maturation approaches, matured TTM-001 and TTM-002 capsid variants (AAV9 capsid variants) with loop IV modifications were generated with significantly enhanced CNS tropism over wild-type AAV9 controls in both NHPs and mice, while also exhibiting de-targeting in peripheral tissues (e.g., the liver and DRG). These resulting matured variants therefore demonstrated cross-species CNS tropism in both NHPs and mice.

### Example 5. Evaluation of TTM-001 and TTM-002 AAV capsid variants in Diverse Primate Species

This Example evaluates the tropism and cross-species compatibility of the TTM-001 (SEQ ID NO: 981 (amino acid) and 983 (DNA), comprising SEQ ID NO: 941) and TTM-002 (SEQ ID NO: 982 (amino acid) and 984 (DNA), comprising SEQ ID NO: 2) capsid variants in two diverse primate species, marmosets (Callithrix jacchus) and African green monkeys (Chlorocebus sabaeus), as compared to their tropism in cynomolgus macaques (Macaca fascicularis) provided in Example 2. The cross-species compatibility and tropism of an AAV9 capsid variant comprising the amino acid sequence of SPHKYG (SEQ ID NO: 966) was also investigated in this example. The amino acid and DNA sequences of TTM-001 and TTM-002 are provided, e.g., in Tables 4 and 5, respectively.

To investigate tropism in African green monkeys, AAV particles comprising the TTM-001 capsid variant, the TTM-002 capsid variant, an AAV9 capsid variant comprising SEQ ID NO: 966, or an AAV9 control under the control of a synapsin promoter, were intravenously injected into NHPs (n=2, 3-12 years of age) at a dose of 2E13 vg/kg. After 14-days in life, the brains and tissues (liver, DRG, quadriceps, and heart) of the NHPs were collected and RNA was extracted. Following RNA recovery and RT-PCR amplification, a systematic NGS enrichment analysis was performed to calculate the fold enrichment ratio relative to the AAV9 wild-type control.

To investigate tropism in marmoset monkeys, AAV particles comprising the TTM-001 capsid variant, the TTM-002 capsid variant, an AAV9 capsid variant comprising SEQ ID NO: 966, or an AAV9 control, were intravenously injected into NHPs (n=2, >10 months of age) at a dose of 2E13 vg/kg (8.75E12 vg/mL). After 28-days in life, the brains and tissues (liver quadriceps, and heart) of the NHPs were collected and RNA was extracted. Following RNA recovery and RT-PCR amplification, a systematic NGS enrichment analysis was performed to calculate the fold enrichment ratio relative to the AAV9 wild-type control.

As provided in Table 63 (African green monkeys) and Table 64 (marmosets), both the TTM-001 and TTM-002 capsid variants demonstrated increased CNS tropism in diverse primate species. The TTM-001 capsid variant demonstrated a 73.6-fold increase in expression relative to AAV9 in the brain of cynomolgus macaques (Table 21, Example 2), a 43.5-fold increase in expression relative to AAV9 in the brain of African green monkeys, and a 703.3-fold increase in expression relative to AAV9 in the brain of marmosets. The TTM-002 capsid variant demonstrated a 62.6-fold increase in expression relative to AAV9 in the brain of cynomolgus macaques (Table 21), a 13.8-fold increase in expression relative to AAV9 in the brain of African green monkeys, and a 366.6-fold increase in expression relative to AAV9 in the brain of marmosets. Both TTM-001 and TTM-002 led to a significant increase in expression relative to AAV9 in the heart of both African green monkeys and marmosets (Table 63 and Table 64). The AAV9 capsid variant comprising SEQ ID NO: 963 also demonstrated in increase in expression relative to AAV9 in the brain and heart of both African green monkeys and marmosets. Furthermore, TTM-001, TTM-002, and the AAV9 capsid variant comprising SEQ ID NO: 966, also all led to increased expression in the brain of both BALB/c and C57B1/6 mice (Table 23, Example 2), demonstrating an average fold change in expression relative to AAV9 across both species of mice of 63.1, 66.8, and 126.97, respectively.

TABLE 63
NGS-Fold Enrichment of TTM-001 (comprises SEQ ID NO: 941),
TTM-002 (comprises SEQ ID NO: 2), and an AAV9 capsid
variant comprising SEQ ID NO: 966 in
African green monkeys

| | SEQ ID | Fold Enrichment relative to AAV9 | | | | | |
|---|---|---|---|---|---|---|---|
| Sequence | NO: | Brain | DRG | Heart | Liver DNA | Liver RNA | Muscle |
| SPHSKA | 941 | 43.525 | 1.010 | 184.789 | 0.242 | 1.547 | 1.715 |
| HDSPHK | 2 | 13.779 | 0.678 | 35.991 | 0.084 | 0.087 | 0.144 |
| SPHKYG | 966 | 9.805 | 0.071 | 44.865 | 0.085 | 0.136 | 0.234 |

TABLE 64
NGS-Fold Enrichment of TTM-001 (comprises SEQ ID NO: 941), TTM-002
(comprises SEQ ID NO: 2), and an AAV9 capsid variant comprising
SEQ ID NO: 966 in Marmosets

| | SEQ ID | Fold Enrichment relative to AAV9 | | | | |
|---|---|---|---|---|---|---|
| Sequence | NO: | Brain | Heart | Liver DNA | Liver RNA | Muscle |
| SPHSKA | 941 | 703.610 | 48.979 | 0.268 | 0.779 | 0.425 |
| HDSPHK | 2 | 366.625 | 18.572 | 0.075 | 0.276 | 0.229 |
| SPHKYG | 966 | 150.209 | 17.232 | 0.045 | 0.014 | 0.146 |

Taken together, these data demonstrate that the AAV9 capsid variants of TTM-001 and TTM-002 demonstrated increased CNS tropism relative to the AAV9 control in the CNS across three diverse primate species and two species of mice, providing evidence of strong cross-species capacity. The AAV9 capsid variant comprising the amino acid sequence of SEQ ID NO: 966 also demonstrated strong CNS expression relative to the AAV9 control in two species of NHPs and two species of mice, also showing strong cross-species capacity.

### Example 6. Advanced Maturation of TTM-002 Capsid Variant in Mice

This Example describes additional maturation of the TTM-002 (SEQ ID NO: 982 (amino acid) and 984 (DNA), comprising SEQ ID NO: 2) capsid variant in mice. In order to mature the TTM-002 capsid variant, sets of three contiguous amino acids were randomized across the mutagenesis region in TTM-002 sequence, which spanned from position 450 to position 466, numbered according to SEQ ID NO: 982. Unlike the maturation performed in in Example 4, where the SPH motif that was observed in the AAV capsid variants that demonstrated the greatest fold-enrichment in the NHP brain relative wild-type AAV9 was not disrupted, in the maturation approach used in this Example, the SPH motif was not held constant to further explore the role of this motif in the capsid variant. The matured TTM-002 capsid variants that resulted from the maturation approach were pooled together for subsequent testing and characterization in mice.

The library of matured AAV capsid variants generated from the TTM-002 matured AAV capsid variant were intravenously injected into the tail vein of three CD-1 Outbred mice (Charles River; 6-8 weeks of age) at a dose of 1.0×10¹² VG/dose. After about 28 days in life, the brains of the mice were isolated, and RNA was extracted. Following RNA recovery and RT-PCR amplification, a systematic NGS enrichment analysis was performed to calculate the fold enrichment ratio relative to the corresponding TTM-002 non-matured control, and the peptides comprised within the variants were identified. Variants were filtered by those with a raw virus count in the sample above 10 and a coefficient of variance (CV) that was greater than 1 (identifies the peptides/variants reliably detected in the majority of the samples isolated from the three mice).

Following the advanced maturation screen and filtering of the variants, 1302 variants demonstrated an increase in expression relative to the non-matured TTM-002 capsid variant in the brain of the outbred mice. Of the 1302 variants with improved tropism relative to the non-matured TTM-002, 1283 comprised the SPH motif in the same position as the non-matured TTM-002 capsid variant (e.g., immediately subsequent to position 455, relative to a reference sequence numbered according to the amino acid sequence of SEQ ID NO: 138 or 982). Mutations in the region of the SPH motif present in the non-matured TTM-002 capsid variant only consistently appear in those variants with a fold change of 0.2 or 0.1 or lower relative to the non-matured TTM-002 control in the brain of the mice. This indicates that the SPH motif may be important to the increased brain tropism that observed for the TTM-002 capsid variant. In instances when the SPH motif was disrupted, the fold change of the matured variants of TTM-002 decreased considerably in relation to the non-matured TTM-002 variant which comprised the SPH motif.

### Example 7. Individual Capsid Characterization of TTM-001, TTM-002, TTM-003, TTM-006, and TTM-027 in NHPs

This example describes the transduction level, tropism, ability to cross the blood brain barrier, and overall spatial distribution in the central nervous system (CNS) and peripheral tissues of the AAV capsid variants TTM-002 (SEQ ID NO: 982 (amino acid) and SEQ ID NO: 984 (DNA), comprising SEQ ID NO: 2), TTM-001 (SEQ ID NO: 981 (amino acid) and SEQ ID NO: 983 (DNA), comprising SEQ ID NO: 941), TTM-003 (SEQ ID NO: 36 (amino acid) and SEQ ID NO: 72 (DNA), comprising SEQ ID NO: 3589), TTM-006 (SEQ ID NO: 39 (amino acid) and SEQ ID NO: 75 (DNA), comprising SEQ ID NO: 3241), and/or TTM-027 (SEQ ID NO: 69 (amino acid) and SEQ ID NO: 9 (DNA), comprising SEQ ID NO: 3272), relative to AAV9 following intravenous administration in African green monkeys (Chlorocebus sabaeus), marmosets (Callithrix jacchus), and/or cynomolgus macaques (Macaca fascicularis).

A. Evaluation of TTM-002 in African Green Monkeys (Chlorocebus sabaeus)

AAV particles were generated with the TTM-002 capsid variant or the AAV9 capsid control which comprised a self-complementary viral genome encoding an histone H2b protein with an HA tag driven by a ubiquitous CBA promoter. The AAV particles comprising the TTM-002 capsid variant or the AAV9 capsid control were administered to the African green monkeys (Chlorocebus sabaeus) (n=2) intravenously at a dose of 1e12 VG/kg or 1e13 VG/kg. The in-life period was 28 days and then various CNS and peripheral tissues were collected for measuring transgene mRNA (expression) by RT-qPCR and viral DNA (biodistribution) by ddPCR.

As shown in Table 27, the TTM-002 capsid variant resulted in increased brain biodistribution in all brain regions investigated as compared to AAV9 at both doses tested. The TTM-002 capsid variant also led to increased transgene expression in the brain relative to AAV9 at both doses tested (Table 28). In the spinal cord, the TTM-002 capsid variant distributed to the cervical spinal cord and the spinal cord ventral horn at a higher level relative to AAV9 (Table 27) and it mediated higher transgene expression than AAV9 in both the full spinal cord and the ventral horn (Table 28). When administered at a dose of 1e13 VG/kg, TTM-002 delivered 1-2 viral genomes (VGs) per cell on average across multiple brain areas, outperforming AAV9 by 4- to 24-fold, and was capable of expressing 16- to 186-fold more transgene RNA (Table 27 and Table 28). The TTM-002 capsid variant exhibited lower biodistribution (Table 27) and transgene expression (Table 28) in the DRG relative to AAV9, indicating that TTM-002 capsid variant was detargeted in the DRG relative to AAV9. Similar expression and distribution were observed by immunohistochemistry performed on these CNS tissues. High-throughput analysis of immunohistochemistry stainings indicated that TTM-002 was capable of targeting upwards of 50% of cells in the brain (FIG. 1A), including both astrocytes and neurons (FIG. 1B). In contrast with the tropism in mice as provided in Example 12, TTM-002 demonstrated a bias towards Sox9 (+) astrocytes over neurons, labeled with either NeuN or SMI311 (FIG. 1B).

Distribution and transgene expression was also measured in the peripheral tissues of the liver, heart, and quadriceps. In the liver, TTM-002 capsid variant exhibited lower biodistribution (Table 27) and transgene expression (Table 28) relative to AAV9, indicating that TTM-002 capsid variant was detargeted in the liver relative to AAV9. In the heart, the TTM-002 capsid variant exhibited comparable levels of biodistribution relative to AAV9 (Table 27), but increased transgene expression relative to AAV9 (Table 28). In the quadriceps, TTM-002 capsid variant exhibited lower biodistribution (Table 27) and lower transgene expression (Table 28), relative to AAV9. Similar expression and distribution were observed by immunohistochemistry performed on these peripheral tissues.

TABLE 27
Quantification of viral genome copies per diploid genome (biodistribution) by ddPCR
following intravenous administration of AAV particles comprising a TTM-002 capsid

| | 1e12 VG/kg | | | 1e13 VG/kg | | |
|---|---|---|---|---|---|---|
| | AAV9 | TTM-002 | | AAV9 | TTM-002 | |
| | (VG copies/ | (VG copies/ | TTM-002 | (VG copies/ | (VG copies/ | TTM-002 |
| | diploid | diploid | relative to | diploid | diploid | relative to |
| Tissue | genome) | genome) | AAV9 | genome) | genome) | AAV9 |
| Putamen | 0.03 | 0.37 | 12.3 | 0.26 | 2.4 | 9.2 |
| Caudate | 0.02 | 0.58 | 29 | 0.14 | 2.1 | 14.7 |
| Thalamus | 0.06 | 0.21 | 3.5 | 0.25 | 1.0 | 4 |
| Hippocampus | 0.03 | 0.29 | 9.7 | 0.16 | 1.56 | 9.8 |
| Substantia | 0.05 | 0.34 | 6.8 | 0.37 | 1.38 | 3.7 |
| Nigra | | | | | | |
| Motor Cortex | 0.03 | 0.56 | 19 | 0.27 | 2.4 | 8.9 |
| Frontal Cortex | 0.04 | 0.67 | 17 | 0.20 | 3.6 | 18 |
| Temporal | 0.03 | 0.31 | 10 | 0.11 | 2.67 | 24 |
| Cortex | | | | | | |
| Cerebral | 0.008 | 0.08 | 10 | 0.03 | 0.16 | 5.3 |
| Cortex | | | | | | |
| Dentate | 0.06 | 0.10 | 1.7 | 0.32 | 3.21 | 10 |
| Nucleus | | | | | | |
| Cervical | 0.03 | 0.12 | 4 | 0.19 | 0.91 | 4.8 |
| Spinal Cord | | | | | | |
| Thoracic | 0.04 | 0.03 | 0.75 | 0.36 | 0.38 | 1.1 |
| Spinal Cord | | | | | | |
| Lumbar Spinal | 0.04 | 0.03 | 0.75 | 0.29 | 0.37 | 1.3 |
| Cord | | | | | | |
| C5 Ventral | 0.04 | 0.25 | 6.3 | 0.29 | 2.2 | 7.6 |
| Horn | | | | | | |
| L5 Ventral | 0.06 | 0.28 | 4.7 | 0.31 | 1.9 | 6.1 |
| Horn | | | | | | |
| Cervical DRG | 0.07 | 0.01 | −7 | 0.81 | 0.36 | −2.3 |
| Thoracic DRG | 0.06 | 0.01 | −6 | 1.31 | 0.43 | −3 |
| Lumbar DRG | 0.07 | 0.01 | −7 | 1.31 | 0.57 | −2.3 |
| Liver | 9.5 | 1.2 | −7.9 | 127 | 7.7 | −16.5 |
| Heart | 0.6 | 0.7 | 1.2 | 5.4 | 5.4 | 1 |
| Quadriceps | 0.2 | 0.06 | −3.3 | 1.7 | 0.6 | −2.8 |

TABLE 28
Quantification of transgene mRNA by RT-qPCR following intravenous
administration of AAV particles comprising a TTM-002 capsid

| | 1e12 VG/kg | | | 1e13 VG/kg | | |
|---|---|---|---|---|---|---|
| | AAV9 | TTM-002 | | AAV9 | TTM-002 | |
| | (transgene | (transgene | | (transgene | (transgene | |
| | mRNA fold | mRNA fold | | mRNA fold | mRNA fold | |
| | over | over | TTM-002 | over | over | TTM-002 |
| | housekeeping | housekeeping | relative | housekeeping | housekeeping | relative |
| Tissue | gene) (2^{-dCT}) | gene) (2^{-dCT}) | to AAV9 | gene) (2^{-dCT}) | gene) (2^{-dCT}) | to AAV9 |
| Putamen | 0.02 | 0.3 | 15 | 0.09 | 4.22 | 47 |
| Caudate | 0.02 | 0.8 | 40 | 0.11 | 4.29 | 39 |
| Thalamus | 0.04 | 0.4 | 10 | 0.4 | 5.8 | 14.5 |
| Hippocampus | 0.02 | 0.4 | 20 | 0.1 | 4.3 | 43 |
| Substantia | 0.1 | 1.2 | 12 | 0.3 | 11.6 | 39 |
| Nigra | | | | | | |
| Motor Cortex | 0.08 | 5.00 | 63 | 0.36 | 21.8 | 61 |
| Frontal | 0.04 | 3.1 | 78 | 0.3 | 27.7 | 92 |
| Cortex | | | | | | |
| Temporal | 0.02 | 0.8 | 40 | 0.1 | 26.9 | 27 |
| Cortex | | | | | | |
| Cerebral | 0.04 | 1.1 | 28 | 0.2 | 17.4 | 87 |
| Cortex | | | | | | |
| Dentate | 0.3 | 0.9 | 3 | 1.8 | 42.0 | 23 |
| Nucleus | | | | | | |
| Cervical | 0.2 | 2.0 | 10 | 0.8 | 20.2 | 25 |
| Spinal Cord | | | | | | |
| Thoracic | 0.13 | 0.25 | 1.9 | 0.7 | 4.8 | 6.9 |
| Spinal Cord | | | | | | |
| Lumbar | 0.4 | 0.5 | 1.3 | 2.2 | 9.2 | 4.2 |
| Spinal Cord | | | | | | |
| C5 Ventral | 0.2 | 1.4 | 7 | 1.7 | 33 | 19 |
| Horn | | | | | | |
| L5 Ventral | 1.1 | 3.4 | 3.1 | 12.4 | 102 | 8.2 |
| Horn | | | | | | |
| Cervical | 3.6 | 1.2 | −3 | 63.1 | 15.9 | −4 |
| DRG | | | | | | |
| Thoracic | 1.8 | 1.3 | −1.4 | 43.9 | 15.7 | −2.8 |
| DRG | | | | | | |
| Lumbar DRG | 1.9 | 1.0 | −1.9 | 34.9 | 27.6 | −1.3 |
| Liver | 0.88 | 0.25 | −3.5 | 2.2 | 0.97 | −2.3 |
| Heart | 8.7 | 42 | 4.8 | 110 | 363 | 3.3 |
| Quadriceps | 9.7 | 1.1 | −8.3 | 59 | 21 | −2.8 |

Taken together, these data demonstrate that TTM-002 is an enhanced CNS tropic capsid in NHPs (African green monkeys) that can infect non-neuronal cells. TTM-002 was also detargeted in the DRG and liver relative to AAV9, but showed increased transgene expression in the heart relative to AAV9. Additionally, the TTM-002 capsid variant was able to successfully penetrate the blood brain barrier following intravenous injection.

B. Evaluation of TTM-001 and TTM-002 in Marmosets (Callithrix jacchus)

AAV particles were generated with the TTM-002 capsid variant, the TTM-001 capsid variant, or the AAV9 capsid control which comprised a self-complementary viral genome encoding a histone H2b protein with an MYC tag (TTM-002 capsid variant), His tag (TTM-001 capsid variant), or HA tag (AAV9 control capsid) driven by a ubiquitous CAG promoter. The AAV particles comprising the TTM-002 capsid variant, the TTM-001 capsid variant, or the AAV9 capsid control were administered to the marmosets (Callithrix jacchus) (n=3) intravenously in a single solution, at the doses indicated in Table 29. The in-life period was 28 days and then various CNS and peripheral tissues were collected for measuring transgene mRNA (expression) by RT-qPCR, protein expression by IHC, and viral DNA (biodistribution) by ddPCR. Data were then normalized to the dose of each viral vector in the dosing solution.

TABLE 29
Titer of the AAV particles comprising the various capsids
in solution dosed in marmosets

| | | Ratio of Capsid |
|---|---|---|
| Capsid Variant | Actual Titer Dosed | Variant to AAV9 |
| TTM-001 | 1.44 × 10¹¹ vg/mL | 0.34 |
| AAV9 | 4.00 × 10¹¹ vg/mL | 1.0 |
| TTM-002 | 4.17 × 10¹¹ vg/mL | 1.0 |

As shown in Table 31, both the TTM-001 and TTM-002 capsid variants demonstrated increased biodistribution in the caudate and motor cortex in the brain of the marmosets relative to the AAV9 control. The TTM-001 and TTM-002 capsid variants also led to increased transgene expression (Table 32) in the caudate and motor cortex in the brain of the marmosets. In fact, biodistribution and transgene expression were increased over 100-400 fold for both TTM-001 and TTM-002 in the brain relative to AAV9. TTM-002 delivered upwards of 280-fold more viral genomes and expressed 500-fold higher transgene RNA levels than AAV9 (Tables 31 and 32). Similar expression and distribution was observed by immunohistochemistry. More specifically, staining for TTM-001 and TTM-002 was detected in the mid-brain, caudate, putamen, thalamus, and cerebellum, and this staining was increased for both capsid variants in each of these brain tissues relative to AAV9. Staining for TTM-001 and TTM-002 was also observed in the molecular and granule layer of the cerebellum.

Biodistribution and transgene expression were also measured in the peripheral tissues of the liver, heart, and quadriceps. In the liver, the TTM-002 capsid variant exhibited lower biodistribution (Table 31) and transgene expression (Table 32) relative to AAV9, indicating that the TTM-002 capsid variant was detargeted in the liver relative to AAV9 in marmosets. The TTM-001 capsid variant demonstrated comparable biodistribution and transgene expression in the liver (Table 31 and Table 32) as well as comparable transgene expression in the heart and muscle (Table 32) relative to AAV9. Both TTM-001 and TTM-002 led to decreased biodistribution (Table 31) relative to AAV9 in the heart and muscle, and TTM-002 also resulted in lower transgene expression in the heart and muscle relative to AAV9 (Table 32).

TABLE 31
Quantification of viral genome copies per diploid genome (biodistribution) by
ddPCR following intravenous administration of AAV particles comprising a TTM-
001 capsid or a TTM-002 capsid normalized to the actual titer of the viral vector
in the dosing solution (vg/dg = viral genome copies/diploid genome)

| | Tissue | | | | | | | | | |
|---|---|---|---|---|---|---|---|---|---|---|
| | Caudate | | Motor Cortex | | Heart | | Muscle | | Liver | |
| | | vg/dg | | vg/dg | | vg/dg | | vg/dg | | vg/dg |
| | | relative | | relative | | relative | | relative | | relative |
| Capsid | vg/dg | to AAV9 | vg/dg | to AAV9 | vg/dg | to AAV9 | vg/dg | to AAV9 | vg/dg | to AAV9 |
| TTM-001 | 1.67 | 142.70 | 2.69 | 124.06 | 0.28 | 0.53 | 0.08 | 0.39 | 14.86 | 0.99 |
| TTM-002 | 3.55 | 294.36 | 5.80 | 264.86 | 0.33 | 0.69 | 0.08 | 0.32 | 6.92 | 0.49 |
| AAV9 | 0.01 | 1.00 | 0.02 | 1.00 | 0.48 | 1.00 | 0.23 | 1.00 | 13.79 | 1.00 |

TABLE 32
Quantification of transgene mRNA by RT-qPCR following intravenous administration of AAV
particles comprising a TTM-001 capsid or a TTM-002 capsid normalized to the actual titer
of the viral vector in the dosing solution (mRNA = transgene mRNA fold over housekeeping
gene; rel. to AAV9 = transgene mRNA fold over housekeeping gene relative to AAV9)

| | Tissue | | | | | | | | | |
|---|---|---|---|---|---|---|---|---|---|---|
| | Caudate | | Motor Cortex | | Heart | | Muscle | | Liver | |
| | | rel. to | | rel. to | | rel. to | | rel. to | | rel. to |
| Capsid | mRNA | AAV9 | mRNA | AAV9 | mRNA | AAV9 | mRNA | AAV9 | mRNA | AAV9 |
| TTM-001 | 17.56 | 594.71 | 27.80 | 586.23 | 16.05 | 1.40 | 0.26 | 1.19 | 3.23 | 1.93 |
| TTM-002 | 14.21 | 479.39 | 19.73 | 410.40 | 2.78 | 0.27 | 0.06 | 0.46 | 0.62 | 0.36 |
| AAV9 | 0.03 | 1.00 | 0.05 | 1.00 | 12.67 | 1.00 | 0.15 | 1.00 | 1.85 | 1.00 |

These data in marmosets for TTM-002 were similar to those observed in African green monkeys, further demonstrating cross-species compatibility of the TTM-002 capsid variant. Taken together, these data demonstrate that TTM-001 and TTM-002 are enhanced CNS tropic capsids in marmosets. TTM-002 was also detargeted in the liver, heart, and muscle relative to AAV9 in marmosets, where TTM-001 demonstrated comparable biodistribution and/or transgene expression in the liver, heart, and muscle compared to AAV9. Additionally, the TTM-001 and TTM-002 capsid variants were able to successfully penetrate the blood brain barrier following intravenous injection.

C. Evaluation of TTM-001, TTM-002, TTM-003, TTM-006, and TTM-027 in Cynomolgus Macaques (Macaca fascicularis)

AAV particles were generated with the TTM-002 capsid variant, the TTM-001 capsid variant, the TTM-003 capsid variant, the TTM-006 capsid variant, the TTM-027 capsid variant, or the AAV9 capsid control which comprised a self-complementary viral genome encoding a histone H2b protein driven by a ubiquitous CAG promoter. The AAV particles comprising the TTM-002 capsid variant, the TTM-001 capsid variant, the TTM-027 capsid variant or the AAV9 capsid control were administered to a first group of male cynomolgus macaques (Macaca fascicularis; 4-6 kg body weight; over 2 years old) intravenously in a single solution, at a total dose per group of 2e13 VG/kg or a dose per capsid of 4e12 VG/kg. The AAV particles comprising the TTM-003 capsid variant or the TTM-006 capsid variant were administered to a second group of male cynomolgus macaques (Macaca fascicularis; 4-6 kg body weight; over 2 years old) intravenously in a single solution, at a total dose per group of 2e13 VG/kg or a dose per capsid of 4e12 VG/kg. The in-life period was 28 days for both groups, and then various CNS and peripheral tissues were collected for measuring transgene mRNA (expression) by RT-qPCR; protein expression by IHC/chromogenic staining (e.g., DAB staining for percent of DAB+ cells indicating the percent of cells transduced); percent positive cells (e.g., neurons, motor neurons, and astrocytes) in brain and spinal cord regions by immunofluorescence microscopy; and viral DNA (biodistribution) by ddPCR.

As shown in Table 34, TTM-001, TTM-002, TTM-003, TTM-006, and TTM-027 demonstrated increased CNS transduction and/or biodistribution in several regions of the brain (greater than 30% of cells transduced observed in several regions for multiple capsid variants) and spinal cord of the cynomolgus macaques after intravenous administration at a relatively low dose of 4e12 vg/kg. More specifically, TTM-003 was capable of transducing up to 40% of cells in the caudate, putamen, and cortex; TTM-027 was capable of transducing up to 30% of cells in the caudate, putamen, and cortex; and both showed improved delivery to the spinal cord relative to AAV9 and TTM-002, at a dose of 4e12 vg/kg.

Cell-typing was also performed in the putamen, substantia nigra, and temporal cortex of the brain to measure the percent of neurons (NeuN+ cells) and astrocytes (Sox9+ cells) that were transduced by the AAV particles comprising the TTM-003 and TTM-027 capsid variants or the AAV9 controls (Table 35). TTM-003 was capable of transducing up to 47.8% of neurons and 79.5% of astrocytes in the putamen; 25.3% of neurons and 87.5% of astrocytes in the temporal cortex; and 33.7% of neurons and 18.6% of astrocytes in the substantia nigra (Table 35). TTM-027 was capable of transducing up to 27% of neurons and 41.8% of astrocytes in the putamen; 12.3% of neurons and 51.4% of astrocytes in the temporal cortex; and 21.1% of neurons and 12.2% of astrocytes in the substantia nigra (Table 35). Co-localization of TTM-027 and TTM-003 with motor neurons (ChAT+ cells) was also observed in the spinal cord by immunofluorescence microscopy (Table 35). Across the lumbar, cervical, and thoracic spinal cord, TTM-003 was capable of transducing 78.5% of motor neurons and TTM-027 was capable of transducing 53.5% of motor neurons (Table 35).

In the peripheral tissues, all of the TTM-001, TTM-002, TTM-003, TTM-006, and TTM-027 capsid variants tested exhibited robust liver de-targeting relative to AAV9 (Table 36).

TABLE 34
Quantification of viral genome copies per diploid genome (vg/dg) (biodistribution) by
ddPCR, transgene mRNA by RT-qPCR (mRNA = transgene mRNA fold over housekeeping
gene), and percent of DAB+ cells in tissues of the CNS of cynomolgus macaques

| | Putamen | | |
|---|---|---|---|
| Capsid | vg/dg | mRNA | % DAB+ Cells |
| AAV9 | 0.1 | 0.03 | 2 |
| TTM-001 | 0.2 | 0.05 | 5 |
| TTM-002 | 0.4 | 0.2 | 13 |
| TTM-027 | 1.2 | 0.83 | 29 |
| TTM-006 | 0.7 | 0.19 | 15 |
| TTM-003 | 1.1 | 1.69 | 39 |
| | Caudate | | |
| Capsid | vg/dg | mRNA | % DAB+ Cells |
| AAV9 | 0.04 | 0.02 | 2 |
| TTM-001 | 0.16 | 0.03 | 5 |
| TTM-002 | 0.42 | 0.15 | 14 |
| TTM-027 | 1.09 | 0.67 | 23 |
| TTM-006 | 0.74 | 0.64 | 27 |
| TTM-003 | 1.09 | 2.99 | 41 |
| | Motor Cortex | | |
| Capsid | vg/dg | mRNA | % DAB+ Cells |
| AAV9 | 0.1 | 0.02 | 1 |
| TTM-001 | 0.3 | 0.02 | 4 |
| TTM-002 | 0.7 | 0.07 | 9 |
| TTM-027 | 1.5 | 1.38 | 22 |
| TTM-006 | 0.8 | 1.36 | 14 |
| TTM-003 | 1.2 | 7.43 | 40 |
| | Cervical (C3) Spinal Cord | | |
| | vg/dg | mRNA | % DAB+ Cells |
| Capsid | (ventral horn) | (ventral horn) | (grey matter) |
| AAV9 | 0.1 | 0.4 | 2 |
| TTM-001 | 0.1 | 0.1 | 2 |
| TTM-002 | 0.3 | 0.5 | 8 |
| TTM-027 | 0.6 | 1.9 | 8 |
| TTM-006 | 0.9 | 3.1 | 15 |
| TTM-003 | 1.0 | 10.7 | 16 |
| | Cervical Dorsal Root Ganglion | | |
| Capsid | vg/dg | mRNA | % DAB+ Cells |
| AAV9 | 0.10 | 3.9 | 1 |
| TTM-001 | 0.04 | 0.3 | 2 |
| TTM-002 | 0.04 | 1.0 | 2 |
| TTM-027 | 0.05 | 1.6 | 1 |
| TTM-006 | 0.18 | 7.4 | 2 |
| TTM-003 | 0.12 | 21.2 | 1 |

TABLE 35
Quantification of neurons (% NeuN positive cells),
motor neurons (% chAT positive cell) and/or
astrocytes (% Sox9 cells) transduced with the
TTM-003 and TTM-027 capsid variants in the putamen,
temporal cortex, substantia nigra, and spinal cord

| | Putamen | |
|---|---|---|
| Capsid | % NeuN Positive Cells | % Sox9 Positive Cells |
| AAV9 (control 1) | 0.2 | 2.7 |
| AAV9 (control 2) | 0.5 | 6.5 |
| TTM-003 | 47.8 | 79.5 |
| TTM-027 | 27.0 | 41.8 |
| | Temporal Cortex | |
| Capsid | % NeuN Positive Cells | % Sox9 Positive Cells |
| AAV9 (control 1) | 0.1 | 2.7 |
| AAV9 (control 2) | 0.1 | 4.9 |
| TTM-003 | 25.3 | 87.5 |
| TTM-027 | 12.3 | 51.4 |
| | Substantia Nigra | |
| Capsid | % NeuN Positive Cells | % Sox9 Positive Cells |
| AAV9 (control 1) | 2.7 | 2.8 |
| AAV9 (control 2) | 1.6 | 7.0 |
| TTM-003 | 33.7 | 18.6 |
| TTM-027 | 21.1 | 12.2 |

| | Lumbar, Cervical, and Thoracic Spinal Cord |
|---|---|
| Capsid | % ChAT Positive Cells |
| AAV9 (control 1) | 14.9 |
| AAV9 (control 2) | 23.8 |
| TTM-003 | 78.5 |
| TTM-027 | 53.5 |

TABLE 36
Quantification of viral genome copies per diploid genome (vg/dg) (biodistribution) by
ddPCR, transgene mRNA by RT-qPCR (mRNA = transgene mRNA fold over housekeeping
gene), and percent of DAB+ cells in peripheral tissues of cynomolgus macaques

| | Liver | | |
|---|---|---|---|
| Capsid | vg/dg | mRNA | % DAB+ Cells |
| AAV9 | 147 | 8.9 | 93 |
| TTM-001 | 15 | 0.2 | 20 |
| TTM-002 | 13 | 0.4 | 32 |
| TTM-027 | 2 | 0.2 | 7 |
| TTM-006 | 9 | 0.4 | 24 |
| TTM-003 | 5 | 0.4 | 24 |
| | Heart | | |
| Capsid | vg/dg | mRNA | % DAB+ Cells |
| AAV9 | 2.2 | 25.8 | 27 |
| TTM-001 | 0.4 | 3.5 | 10 |
| TTM-002 | 1.0 | 8.0 | 20 |
| TTM-027 | 0.6 | 3.4 | 9 |
| TTM-006 | 0.7 | 9.0 | 20 |
| TTM-003 | 0.9 | 23.6 | 16 |

| | Muscle (Vastus Lateralis) | |
|---|---|---|
| Capsid | vg/dg | mRNA |
| AAV9 | 0.58 | 1.38 |
| TTM-001 | 0.09 | 0.04 |
| TTM-002 | 0.24 | 0.08 |
| TTM-027 | 0.21 | 0.05 |
| TTM-006 | 0.86 | 9.84 |
| TTM-003 | 1.01 | 10.85 |
| | Muscle (Gastrocnemius) | |
| Capsid | vg/dg | mRNA |
| AAV9 | 0.56 | 2.13 |
| TTM-001 | 0.08 | 0.05 |
| TTM-002 | 0.22 | 0.13 |
| TTM-027 | 0.18 | 0.05 |
| TTM-006 | 0.51 | 1.04 |
| TTM-003 | 0.58 | 1.57 |

Taken together, these data demonstrate that TTM-001, TTM-002, TTM-003, TTM-006, and TTM-027 are enhanced CNS tropic capsids in cynomolgus macaques that were capable of crossing the blood brain barrier following intravenous injection, even at a low dose of 4e12 vg/kg. TTM-003 and TTM-027 were also capable of transducing both neurons and astrocytes in several brain tissues as well as motor neurons in the spinal cord. All capsids variants also demonstrated robust liver de-targeting relative to AAV9.

D. Individual Evaluation of TTM-003 in Cynomolgus Macaques (Macaca fascicularis)

AAV particles were generated with the TTM-003 capsid variant which comprised a self-complementary viral genome encoding a histone H2b protein with an HA tag driven by a ubiquitous CAG promoter. The AAV particles comprising the TTM-003 capsid variant were administered to cynomolgus macaques (Macaca fascicularis) (n=3 male monkeys; 4-12 years of age) intravenously at a dose of 3e13 VG/kg. The in-life period was 28 days and then various CNS and peripheral tissues were collected for measuring transgene mRNA (expression) by RT-ddPCR, viral DNA (biodistribution) by ddPCR, and immunohistochemistry (IHC)/chromogenic quantification of percent positive cells in various tissues (cellular tropism).

As shown in Table 40, substantial and widespread transduction of TTM-003 was observed in the brain and spinal cord in NHPs following intravenous administration of the AAV particles comprising the TTM-003 capsid variant. When administered at a dose of 3e13 VG/kg, TTM-003 was capable of transducing multiple cell types in the brain and spinal cord, including neurons and astrocytes, as quantified in Table 40. Distribution and transgene expression was also measured in the peripheral tissues of liver, heart, and muscle as provided in Table 41.

Taken together, these data demonstrate that TTM-003 is an CNS tropic capsid in NHPs (cynomolgus macaques) that can infect both neuronal and non-neuronal cells. Additionally, the TTM-003 capsid variant was able to successfully penetrate the blood brain barrier following intravenous injection.

TABLE 40
Quantification of viral genome copies per diploid genome (vg/dg) (biodistribution) by
ddPCR; transgene mRNA expression by RT-ddPCR (mRNA = transgene mRNA expression
relative to a housekeeping gene (mTBP)); and the percentage of cells transduced with the TTM-
003 capsid variant measured by co-localized staining of HA (payload tag) and DAB, NeuN
(neurons), or Sox9 (astrocytes) in the brain and spinal cord of NHPs (each value represents the
average from 3 NHPs)

| | | | % HA and | | |
|---|---|---|---|---|---|
| | | | DAB Positive | % HA and NeuN | % HA and Sox9 |
| Tissue | vg/dg | mRNA | Cells | Positive Neurons | Positive Astrocytes |
| Frontal Cortex | 7.5 | 1.4 | 40 | 8.3 | 86.2 |
| Motor Cortex | 6.5 | 7.4 | 60 | 13.7 | 88.4 |
| Temporal Cortex | 4.5 | 0.9 | 47 | 13.4 | 95.1 |
| Entorhinal Cortex | — | — | 34 | 5.9 | 87.8 |
| Hippocampus | 3.3 | 1.5 | 34 | 11.2 | 79.2 |
| Thalamus | 7.6 | 3.1 | 46 | 40.8 | 91.8 |
| Caudate | 7 | 2.5 | 55 | — | — |
| Putamen | 5.3 | 1.8 | 52 | 30.8 | 88 |
| Substantia Nigra | 3.1 | 1.5 | 19 | 13.1 | 45.5 |
| Lateral Geniculate | — | — | 74 | — | — |
| Nucleus (LGN) | | | | | |
| Dentate Nucleus | 2.1 | 1.9 | 38 | — | — |
| Cerebellar Cortex | 1.4 | 2.9 | — | — | — |
| Spinal Cord-Cervical | 4.3 | 8.2 | 20 | — | 78.4 |
| Spinal Cord-Thoracic | — | — | 28 | — | 85.9 |
| Spinal Cord-Lumbar | — | — | 24 | — | 83.8 |
| DRG-Cervical | 2.1 | 5.1 | 62 | — | — |
| DRG-Thoracic | 6.2 | 0.9 | 71 | — | — |
| DRG-Lumbar | 2.6 | 4.8 | 65 | — | — |

TABLE 41
Quantification of viral genome copies per diploid genome (vg/dg) (biodistribution) by
ddPCR; transgene mRNA expression by RT-ddPCR (mRNA = transgene mRNA relative to a
housekeeping gene (mTBP)); and percentage of cells transduced with the TTM-003 capsid
variant measured by co-localized HA (payload tag) and DAB staining in the peripheral tissues
of NHPs (each value represents the average of three individual NHPs)

| Tissue | vg/dg | mRNA | % HA and DAB Cells |
|---|---|---|---|
| Liver | 52.2 | 0.4 | 48 |
| Heart | 5.3 | 67 | 40 |
| Vastus Lateralis | 1.4 | 0.7 | 43 |
| Gastrocnemius | 1.6 | 10.6 | 39 |

E. Evaluation of TTM-027 in Cynomolgus Macaques (Macaca fascicularis)

AAV particles were generated with the TTM-027 capsid variant or the AAV9 capsid control which comprised a self-complementary viral genome encoding a histone H2b protein driven by a ubiquitous CAG promoter. The AAV particles comprising the TTM-027 capsid variant or the AAV9 capsid control were administered to a group of male cynomolgus macaques (Macaca fascicularis; 8-9 kg body weight; 4-10 years old; n=3) intravenously in a single solution, at a total dose per group of 2e13 VG/kg or a dose per capsid of 4e12 VG/kg. The in-life period was 28 days, and then various CNS and peripheral tissues were collected for measuring transgene mRNA (expression) by RT-ddPCR; protein expression by IHC/chromogenic staining and quantification of percent positive cells; and viral DNA (biodistribution) by ddPCR.

As shown in Table 42, TTM-027 demonstrated increased CNS transduction in several brain regions and the spinal cord of the cynomolgus macaques after intravenous administration at a relatively low dose of 4e12 vg/kg.

TABLE 42
Quantification of percent DAB+ cells; viral genome copies per diploid genome (vg/dg)
(biodistribution) by ddPCR; and transgene mRNA expression by RT-ddPCR (mRNA =
transgene mRNA expression relative to a housekeeping gene (mTBP)) in tissues of the CNS of
cynomolgus macaques

| | % DAB Positive Cells | | |
|---|---|---|---|
| | | | % DAB positive cells |
| Tissue | AAV9 | TTM-027 | relative to AAV9 |
| Caudate | 2 | 46 | 23 |
| Putamen | 2 | 45 | 22.5 |
| Thalamus | 2 | 36 | 18 |
| Substantia Nigra | 2 | 15 | 7.5 |
| Hippocampus | 1 | 32 | 32 |
| Entorhinal Cortex | 2 | 30 | 15 |
| Temporal Cortex | 1 | 41 | 41 |
| Primary Motor Cortex | 4 | 47 | 11.75 |
| Dentate Nucleus | 8 | 37 | 4.63 |
| Lateral Geniculate Nucleus (LGN) | 8 | 55 | 6.88 |
| Cervical Spinal Cord | 10 | 22 | 2.2 |
| Thoracic Spinal Cord | 14 | 20 | 1.43 |
| Lumbar Spinal Cord | 13 | 21 | 1.62 |
| Cervical DRG | 57 | 59 | 1.04 |
| Thoracic DRG | 50 | 49 | 0.98 |
| Lumbar DRG | 51 | 58 | 1.14 |
| | vg/dg | | |
| Tissue | AAV9 | TTM-027 | vg/dg relative to AAV9 |
| Caudate | 0.1 | 2.9 | 29.00 |
| Putamen | 0.1 | 2.3 | 23.00 |
| Primary Motor Cortex | 0.2 | 2.4 | 12.00 |
| Cervical DRG | 1.4 | 0.1 | 0.07 |
| | mRNA | | |
| Tissue | AAV9 | TTM-027 | mRNA relative to AAV9 |
| Caudate | 0.046 | 2.5 | 54.35 |
| Putamen | 0.1 | 1.0 | 10.00 |
| Cervical DRG | 3.5 | 0.3 | 0.09 |
| Cervical Spinal Cord | 0.4 | 3.0 | 7.50 |

The percentage of DAB positive cells, biodistribution, and mRNA expression were also quantified in the peripheral tissues of the liver, heart, and muscle, which is provided in Table 43. The TTM-027 capsid variant exhibited robust liver de-targeting relative to AAV9 (Table 43).

TABLE 43
Quantification of percent DAB+ cells; viral genome copies per diploid genome (vg/dg)
(biodistribution) by ddPCR; and transgene mRNA expression by RT-ddPCR (mRNA =
transgene mRNA expression relative to a housekeeping gene (mTBP)) in the peripheral tissues
of cynomolgus macaques

| Tissue | % DAB Positive Cells | | |
|---|---|---|---|
| | AAV9 | TTM-027 | % DAB positive cells relative to AAV9 |
| Liver | 65 | 11 | 0.17 |
| Heart | 19 | 15 | 0.79 |
| Gastrocnemius Muscle | 9 | 16 | 1.78 |
| Vastus Lateralis | 21 | 13 | 0.62 |
| Tissue | vg/dg | | |
| | AAV9 | TTM-027 | vg/dg relative to AAV9 |
| Liver | 113.9 | 1.0 | 0.01 |
| Heart | 2.2 | 0.1 | 0.05 |
| Gastrocnemius Muscle | 1.07 | 0.04 | 0.04 |
| Vastus Lateralis | 1.7 | 0.1 | 0.06 |
| Tissue | mRNA | | |
| | AAV9 | TTM-027 | mRNA relative to AAV9 |
| Liver | 19.6 | 2.1 | 0.11 |
| Heart | 33.0 | 1.1 | 0.03 |
| Gastrocnemius Muscle | 6.29 | 0.51 | 0.08 |
| Vastus Lateralis | 0.51 | 0.02 | 0.04 |

Taken together, these data demonstrate that TTM-027 is an enhanced CNS tropic capsid in cynomolgus macaques that was capable of crossing the blood brain barrier following intravenous injection, even at a low dose of 4e12 vg/kg, which is consistent with what was observed in Example 7C above.

F. Individual Evaluation of TTM-027 in Cynomolgus Macaques (Macaca fascicularis)

AAV particles were generated with the TTM-027 capsid variant which each comprised a self-complementary viral genome encoding a histone H2b protein with an HA tag driven by a ubiquitous CAG promoter. The AAV particles comprising the TTM-027 capsid variant were administered to cynomolgus macaques (Macaca fascicularis) (n=3 male monkeys; 7.4-11 years of age) intravenously at a dose of 3e13 VG/kg. The in-life period was 28 days and then various CNS and peripheral tissues were collected for measuring transgene mRNA (expression) by RT-qPCR, viral DNA (biodistribution) by ddPCR, and immunohistochemistry (IHC)/chromogenic and immunofluorescent quantification of percent positive cells in various tissues (cellular tropism).

As shown in Table 44, substantial and widespread transduction of TTM-027 was observed in the brain and spinal cord of NHPs following intravenous administration of the AAV particles comprising the TTM-027 capsid variant. More specifically, TTM-027 demonstrated superior viral genome biodistribution in a variety of CNS tissues and regions, broader expression in the CNS as shown by both transgene mRNA expression and IHC (Table 44 and Table 45), as well as a highly neurotropic and astrocytic tropism in the brain and the spinal cord (Table 45 and Table 46). TTM-027 when administered intravenously at a dose of 3e13 vg/kg was capable of transducing up to 21-65% of neurons (HA and NeuN positive cells) and 87-97% of astrocytes (HA and Sox9 positive cells) in multiple brain regions; up to 96% of Purkinje Neurons in the cerebellum; up to 84-94% of motor neurons (HA and ChAT positive cells) in the spinal cord; and up to 93-97% of astrocytes (HA and Sox9 positive cells) in the spinal cord (Table 45). TTM-027 was also able to transduce 97.9% of the dopaminergic neurons in the substantia nigra, as indicated by cells that were positive for both tyrosine hydroxylase (TH) and HA (payload tag). The TTM-027 capsid variant was well tolerated in the NHPs.

TABLE 44
Quantification of viral genome copies per diploid genome (vg/dg) (biodistribution) by
ddPCR; transgene mRNA expression by RT-qPCR (mRNA = transgene mRNA expression
relative to a housekeeping gene (mTBP)); and the percentage of cells transduced with the TTM-
027 capsid variant measured by co-localized staining of HA (payload tag) and DAB in the brain
and spinal cord of NHPs (each value represents the average from 3 NHPs; for regions of the
DRG, sensory neuron data are shown)

| Tissue | vg/dg | mRNA | % HA and DAB Positive Cells |
|---|---|---|---|
| Frontal Cortex | 6.1 | 6.1 | 53.8 |
| Motor Cortex | 7.3 | 3.1 | 68.1 |
| Temporal Cortex | 1.7 | 3.8 | 51.8 |
| Hippocampus | 5.4 | 1.8 | 34.7 |
| Thalamus | 8.7 | 5.1 | 50.3 |
| Caudate | 7.6 | 5.7 | 57.5 |
| Putamen | 6.0 | 1.8 | 59.1 |
| Substantia Nigra | 2.6 | 5.4 | 37.9 |
| Dentate Nucleus | 2.7 | 17.7 | 55.2 |
| Cervical Spinal Cord | 5.3 | 5.9 | 25.0 |
| Lumbar Spinal Cord | 4.4 | 11.4 | 21.3 |
| Cervical DRG | 1.8 | 4.1 | 45.5 |
| Thoracic DRG | 6.9 | 3.1 | 41.0 |
| Lumbar DRG | 2.5 | 3.7 | 41.6 |

| Tissue | % DAB Positive Cells |
|---|---|
| Entorhinal Cortex | 41.0 |
| Lateral geniculate nucleus (LGN) | 74.5 |
| Thoracic Spinal Cord | 23.6 |
| Purkinje Neurons | 95.7 |
| Pontine Nuclei | 52.7 |
| Inferior Olivary Nuclei | 42.6 |

TABLE 45
Quantification of the percentage of cells transduced with the TTM-027 capsid variant
measured by co-localized staining of HA (payload tag) and either NeuN (neurons) or Sox9
(astrocytes) in the brain of NHPs (each value represents the average from 3 NHPs)

| | % HA and NeuN Positive | % HA and Sox9 Positive |
|---|---|---|
| Tissue | Neurons | Astrocytes |
| Caudate | 58 | 99 |
| Entorhinal Cortex | 25 | 97 |
| Frontal Cortex | 42 | 99 |
| Hippocampus | 21 | 87 |
| Motor Cortex | 43 | 98 |
| Putamen | 58 | 95 |
| Substantia Nigra | 51 | 89 |
| Temporal Cortex | 31 | 97 |
| Thalamus | 66 | 97 |

TABLE 46
Quantification of the percentage of cells transduced with the TTM-027 capsid variant
measured by co-localized staining of HA (payload tag) and either ChAT (motor neurons) or
Sox9 (astrocytes) in the gray matter of the spinal cord of NHPs (each value represents the
average from 3 NHPs)

| | % HA and ChAT Positive | % HA and Sox9 Positive |
|---|---|---|
| Tissue | Neurons | Astrocytes |
| Cervical Spinal Cord (C2) | 84 | 96 |
| Thoracic Spinal Cord (T8) | 86 | 97 |
| Lumbar Spinal Cord (L2) | 94 | 92 |

The biodistribution and mRNA expression following intravenous administration of AAV particle comprising the TTM-027 capsid variant at a dose of 3e13 vg/kg was also measured in the peripheral tissues of the liver, heart, and muscle (vastus lateralis and gastrocnemius) as provided in Table 47). TTM-027 showed very low biodistribution and mRNA expression in the liver in NHPs (Table 47), and demonstrated substantially reduced liver tropism.

TABLE 47
Quantification of viral genome copies per diploid genome
(vg/dg) (biodistribution) by ddPCR, transgene
mRNA expression relative to a housekeeping gene (mTBP)
by RT-ddPCR in the peripheral tissues, and percent DAB+
cells of NHPs (each value represents the average from 3 NHPs)

| | | Transgene mRNA | % DAB |
|---|---|---|---|
| | | expression relative to a | Positive |
| Tissue | vg/dg | housekeeping gene (mTBP) | Cells |
| Liver | 13.9 | 0.5 | 56.1 |
| Heart | 2.7 | 8.0 | 38.2 |
| Vastus Lateralis | 1.5 | 2.2 | 18.3 |
| Gastrocnemius | 2.2 | 9.6 | 38.9 |

Taken together, the individual characterization of the TTM-027 capsid variant further demonstrates and confirms that the TTM-027 is an enhanced CNS tropic capsid in cynomolgus macaques, capable of crossing the blood brain barrier following intravenous injection, consistent with what was observed in Examples 7C and 7E above.

### Example 8: SOD1 mRNA Knockdown by Intravenous Administration of AAV

In this Example, primary endpoints (body weight, neurological scoring, survival, functional endpoints, and immunohistochemistry) following SOD1 mRNA knockdown by intravenous delivery of AAV were assessed in a SOD1^{G93A} transgenic mouse ALS model. This mouse model expresses a large amount of mutant SOD1 and exhibits adult-onset neurodegeneration of spinal motor neurons and progressive motor deficits leading to paralysis.

Briefly, B6.Cg-Tg (SOD1^{G93A}) 1 Gur mice and wild-type non-carrier mice aged about 52 days were balanced for sex, age, copy number, and body weight. Mice were intravenously administered an SOD1 miRNA-encoding AAV particle with a VOY101 capsid and a viral genome (SEQ ID NO: 109, Table 15) encoding the SOD1 targeting modulatory polynucleotide miR104-788.2 (SEQ ID NO: 2562, Table 14) under the control of an H1 promoter (AAV_VOY101.SOD1). To assess vector distribution and pharmacodynamic effects of SOD1 miRNA, upper and lower regions of the cervical, thoracic, and lumbar spinal cord were harvested at necropsy, frozen in liquid nitrogen, and stored at −80° C. until use. The lower region of each segment was homogenized, aliquoted, and both whole cell DNA and total RNA were independently isolated from the same homogenate. The experimental design is summarized in Table 28.

TABLE 28
Experimental design.

| | | | Dose | Volume | | |
|---|---|---|---|---|---|---|
| Group | Genotype | Test article | (VG/kg) | (μl) | N | Readout |
| WT-V | WT-B6SJL mice (B6.Cg- | Vehicle | 0 | 100 | 10 M/10 F | Body |
| | Tg(SOD1^{G93A})1Gur non- | | | | | weight, |
| | carriers) | | | | | survival, |
| V | B6.Cg-Tg(SOD1^{G93A})1Gur | Vehicle | 0 | 100 | 12 M/12 F | motor |
| C | B6.Cg-Tg(SOD1^{G93A})1Gur | AAV_VOY101.SOD1 | 2E13 | 100 | 12 M/12 F | endpoints, |
| A | B6.Cg-Tg(SOD1^{G93A})1Gur | AAV_VOY101.SOD1 | 6.3E12 | 100 | 12 M/12 F | IHC |
| B | B6.Cg-Tg(SOD1^{G93A})1Gur | AAV_VOY101.SOD1 | 2E12 | 100 | 12 M/12 F | |

### Spinal Cord Vector Genome Distribution

Distribution of the SOD1 miRNA AAV particle after intravenous administration in SOD1^{G93A} transgenic mice was assessed using a multiplex ddPCR assay against the transgene and host targets. As shown in FIGS. 2A-2C, nearly identical vector distribution was observed for each treatment group in all spinal cord segments assessed (lower cervical, lower thoracic, and lower lumbar spinal cord). There was a 2-fold difference in mean vector distribution between the 2E13 and 6.3E12 vg/kg groups (Groups C and A) in all spinal cord segments, and a significant difference between the 2E13 and 2E12 vg/kg Groups (Groups C and B; 1-way ANOVA/Tukey's multiple comparisons). The difference in mean vector distribution between the 6.3E12 and 2E12 vg/kg groups (Groups A and B) was at least 6-fold.

SOD1 mRNA Reduction in Spinal Cord

FIGS. 3A-3C show the amount of human SOD1 mRNA reduction in the spinal cord of SOD1^{G93A} transgenic mice after intravenous administration of the SOD1 miRNA AAV particle. A dose-dependent hSOD1 mRNA reduction was observed for the range of dosages tested (2E12 to 2E13 vg/kg). There was a significant decrease in hSOD1 knockdown between the 2E13 and 6.3E12 vg/kg groups (Groups C and A, respectively) and the control group (V) in all spinal cord regions tested, and between the 2E13 and 2E12 vg/kg groups (Groups C and B, respectively; 1-way ANOVA and Tukey's multiple comparisons). The amount of knockdown was about 70% in the 2E13 vg/kg group (Group C), about 50% in the 6.3E12 vg/kg group (Group A), and about 20% in the 2E12 vg/kg group (Group B), with a similar amount of reduction in hSOD1 mRNA for each group across spinal cord regions.

Efficacy of SOD1 miRNA in Mouse ALS Model

Effects of the SOD1 miRNA AAV particle delivered intravenously on various features of the SOD 1^{G93A} transgenic mouse model were tested.

As shown in FIGS. 4A-4C, there was a clear correlation between SOD1 mRNA level and vector distribution over 2 logs of administered vector for all three spinal cord regions assessed. About 75% knockdown was observed in the cervical and thoracic spinal cord (slightly higher than in the lumbar spinal cord), with an IC50 (~62.5% KD) of about 2 vg/dg based on a 4-parameter nonlinear curve fit (IC50 lower cervical: 2.01 vg/dg, range: 1.58-2.61 vg/dg; IC50 lower thoracic: 1.86 vg/dg, range: 1.38-2.55 vg/dg); IC50 lower lumbar: 3.21 vg/dg, range: 2.54-4.42 vg/dg).

Functional outcomes were also assessed in SOD1^{G93A} mice intravenously administered the SOD1 miRNA AAV particle.

Neuroscore composite rating assessments were performed on female and male SOD1G93A mice. This protocol encompassing a simple neurological scoring system designed to assess motor performance (including circling, clasping, motility, general condition, righting reflex, paw placement, contralateral reflex, visual forepaw reaching, and contralateral rotation), with a scale ranging from 0-4, with 0=no deficit, 1=first symptoms, 2=onset of paresis, 3=paralysis, and 4=human endpoint. A dose-dependent improvement was observed in Neuroscore composite rating in both males and females, although the dose-dependency was more pronounced in female mice (FIG. 5). Male mice may have exhibited a more severe phenotype and progressed pathology at the time of AAV particle administration, resulting in heightened sensitivity to SOD1 miRNA expression and a bell-shaped efficacy curve.

Similarly, improvement in survival was observed in female, male, and all SOD1^{G93A} mice intravenously administered the SOD1 miRNA AAV particle (FIGS. 6A-6C). In female mice, median survival was 140 days for the vehicle-treated group and 139 days for the 2E12 vg/kg group. Median survival for the 6.3E12 vg/kg and 2E13 vg/kg groups could not be calculated at age 300 days due to >50% of the cohort remaining at this time point. There were surviving female mice for all doses tested at age day 300, with the 6.3E12 vg/kg group and 2E13 vg/kg group showing a similar higher probability of survival than the 2E12 vg/kg group (FIG. 6A). In male mice, median survival was 133 days for the vehicle-treated group, 124 (n.s.) and 145 (n.s.) days for the 2E13 vg/kg and 2E12 vg/kg groups, respectively, and 263 (** p=0.003) days for the 6.3E12 vg/kg group. In males, the 6.3E12 vg/kg group showed the greatest number of surviving mice at age 300 days, followed by the 2E13 vg/kg group, whereas all 2E12 vg/kg mice had died by about age 280 days (FIG. 6B). Statistical analysis was performed using the log-rank (Mantel-Cox) test. When combining the data for both males and females, while vehicle-administered SOD1^{G93A} mice died before age 160 days, surviving mice were observed for all SOD1 miRNA AAV particle groups, with the largest number of mice surviving at age 280 days in the 6.3E12 vg/kg group. The least number of surviving mice was observed with the 2E12 vg/kg group at age 280 days (FIG. 6C).

Grip strength was also assessed (FIGS. 7A, 7B, 8A, 8B). Grip strength provides a measure of forepaw and hindlimb muscle strength by measuring the forced required to pull the mouse off a narrow bar or grid. The apparatus used to test grip strength consists of a grasping bar connected to a force transducer. Each animal underwent five trials, and the mean value was calculated. Mice that are no longer able to grab the bar or grid due to disease progression were removed from the experiment, as was the case in the 2E13 vg/kg treatment group in both males and females. In both female and male SOD1^{G93A} mice, the intermediate dose of 6.3E12 vg/kg had the strongest effect of maintaining near wild-type levels of grip strength for both forelimb and all limbs combined at day 130 of age. In female mice, the 2E12 vg/kg dose showed no improvement in grip strength, with a trend similar to vehicle administered mice, while in male mice, a clear improvement in grip strength was observed at this dose. These data are consistent with other behavioral findings, suggesting female mice are more responsive to higher doses of AAV particle, while male mice are more susceptible to overexpression of SOD1 miRNA and do not tolerate the highest dose (2E13 vg/kg).

Overall, these studies demonstrated robust SOD1 knockdown throughout the rostral-caudal extent of the spinal cord that correlated with vector genome levels and significant improvements in motor performance and survival extension. These results suggest that the combination of potent and tolerable AAV miRNA mediated knockdown with intravenous dosing of a BBB-penetrant capsid demonstrates substantial phenotypic rescue in an SOD1-ALS mouse model.

### Example 9: SOD1 mRNA Knockdown by SOD1 miRNA Expressed Under Different Promoters

In this example, SOD1 mRNA knockdown was assessed by measuring the remaining SOD1 mRNA in HEK293T and SH-SY5Y cell lines. Briefly, HEK293T and SH-SY5Y cells were plated at 15,000 cell density in 96-well plate. Next day, cells were treated with SOD1 miRNA-encoding AAV particle with a AAV2 capsid and a viral genome (Table 16 and 17) encoding the SOD1 targeting modulatory polynucleotide miR104-788.2 (SEQ ID NO: 2562, Table 14) under the control of an H1 promoter (AAV_VOY101.SOD1) (Table 16) or other Pol II promoters (CBA, miniCBA, miniEF1AV2, PGK, MP84, hSYN, CBA_CpG depleted exon, miniCBA_CpG depleted cxon, miniEF1AV2_CpGdepleted exon, PGK_CpG depleted exon, MP84_CpGdepleted exon and hSYN_CpG depleted exon, the sequences are included in Table 17). Two days after AAV particle treatment, the cells were harvested and total RNA were extracted to measure the SOD1 mRNA levels by qRT-PCR. We observed a dose dependent SOD1 mRNA knockdown (KD) in both HEK293T and SH-SY5Y cells. SOD1 KD was the highest for miRNAs that were expressed under H1 (Pol III) promoter with almost 95% KD at both MOIs tested (FIG. 9). For the Pol II promoters that we tested, the KD levels were the following: CBA=CBA_CpG depleted exon>miniEF1AV2=miniEF1AV2_CpGdepleted exon>PGK=PGK_CpG depleted exon>miniCBA=miniCBA_CpGdepleted exon>hSYN=hSYN_CpG depleted exon>MP84=MP84_CpG depleted (FIG. 9). A significant difference was not observed between the transgenes that has full CpG amount and decreased CpG amount (for the transgenes that have depleted CpG in exonic regions of the transgenes) (FIG. 9).

### Example 10 SOD1 mRNA Knockdown by Intravenous Administration of AAV

In this example, primary endpoints (vector genome tissue distribution, vector genome cellular tropism, and SOD1 mRNA reduction in various tissues) will be assessed following intravenous administration of the AAV capsid variants described herein. Briefly, cynomolgus macaques (Macaca fascicularis) aged 2-5 years and weighing 2-4 kg will be used for the study. Approximately three weeks before the dosing date, 1 ml of blood will be collected for serum preparation from a sufficient number of animals with normal physical examinations, clinical chemistries, and complete blood counts (CBCs) for a neutralizing antibody (Nab) screening using standard protocols. The serologically qualified animals will be assigned to the study. Animals will be randomized across low (1×10¹² vg/kg), mid (3×10¹² vg/kg), and high (1×10¹³ vg/kg) dose groups for each capsid variant, as well as a vehicle control. Each subject will receive a single intravenous infusion via saphenous or cephalic vein using an infusion pump on Day 0, with a target infusion rate of 5 ml/kg/hour. A selected group of tissues will be harvested on day 28 post AAV administration, with special attention being taken for the processing of the brain and spinal cord. To assess vector distribution and pharmacodynamic effects of SOD1 miRNA, cervical, thoracic, and lumbar spinal cord sections will be harvested. To assess cellular tropism of the miRNA, the even numbered cervical, thoracic, and lumbar spinal cord sections will be harvested. Additional CNS regions/tissues will be assessed (e.g., motor cortex and brainstem), along with an extensive list of peripheral tissues. Distribution of the SOD1 miRNA AAV particle will be assessed using a multiplex ddPCR assay against the transgene and host targets. In situ hybridization will be utilized to detect cellular distribution of the miRNA and resulting SOD1 reduction.

### Example 11. Tropism of TTM-002 AAV Capsid Variant

This Example further investigates the tropism and CNS cells transduced by the TTM-002 capsid variant (SEQ ID NO: 982 (amino acid) and 984 (DNA), comprising SEQ ID NO: 2), as outlined in Table 3 above. The amino acid and DNA sequences of TTM-002 are provided, e.g., in Tables 4 and 5, respectively.

AAV particles were generated with the TTM-002 capsid variant encapsulating a GFP transgene (AAV_TTM-002.GFP) or a payload driven by a heterologous CBA constitutive promoter (AAV_TTM-002.Payload).

Two tandem single cell RNA sequencing runs (scRNA-Seq) of mouse cells derived from the midbrain area were performed. In the first run, cells were pooled from two mice at day 28 post treatment with AAV_TTM-002.Payload particles. In the second run we treated with AAV_TTM-002.GFP particles, in the same manner but without xenografts. Orthotopic xenografts of MDA-MB-361-Luc #1 high passage cells grown as tumorspheres (in tumorsphere media; Sigma #C-28070) were injected (250,000 cells/2 μL/mouse) intracranially into 2-month old female SCID CB17 (Mutation: Icr-Prkdcscid/IcrIcoCrl) congenic immunodeficient mice (Charles River Laboratories). The injections were 2.5 mm (lateral),−1 mm (posterior) with respect to bregma, lowered-3 mm ventral and raised+0.5 mm dorsal to a final-2.5 mm ventral position. Two days later, dilutions of the AAV_TTM-002. Payload particles (run 1), or in the case without xenografts, dilutions of AAV_TTM-002.GFP particles (run 2) were prepared. IV injections of 100 μL (2.5e11 VG/animal) of the AAV_TTM-002.payload particles or AAV_TTM-002.GFP particles were administered through the tail veins of mice (n=5 mice per groups). At 7 days post-injection, mice from run 1 were imaged in an AmiHTX (Spectral Imager) for bioluminescence of the human tumor cells due to expression of luciferase in response to intraperitoneal luciferin injections.

At 28 days post-injection with the AAV_TTM-002.payload particles or AAV_TTM-002.GFP particles, two mice from each run were necropsied, brain samples were isolated, and the midbrain was dissected and isolated. The midbrain samples were then exposed to a cold protease inhibitor (Creative Biomart #NATE-0633) and were dissociated at 6 degrees centigrade. For the samples collected from the mice of run 1 (AAV_TTM-002.Payload particles), myelin depletion was performed (Miltenyi, #130-096-731), cells were filtered through a 40 μM mesh to filter out neurons) and loaded on a 10X chromium G chip. scRNA-Seq was performed (10X Genomics) and samples were sequenced on a NextGen500 Sequencing machine (Illumina). For the samples collected from run 2 (AAV_TTM-002.GFP particles and no xenografts), the cells were not myelin depleted or filtered through 40 μM mesh to include neurons. The cells isolated after run 2 were FACS sorted for GFP+/7AAD-(live GFP+ cells). The resultant cells were loaded on a 10X chromium G chip and the scRNA-Seq was run and processed (10X Genomics).

For run 1, the scRNA-Seq data was filtered to include cells with only greater than 10⁰⁰ genes per cell and less than 5000, and less than 20 percent mitochondrial gene expression. For run 2, the scRNA-Seq data was filtered to include cells with only greater than 200 genes per cell and less than 5000, and less than 20 percent mitochondrial gene expression. The data were normalized, scaled, and integrated into one combined dataset. Clusters were generated with a resolution of 0.3 and each cluster identity was determined using a panel of cell type specific genes (e.g., as described in Brown et al., 2021. “Deep Parallel Characterization of AAV Tropism and AAV-Mediated Transcriptional Changes via Single-Cell RNA Sequencing”. Front. Immunol. 12:730825; the contents of which are hereby incorporated by reference in its entirety). The percentage of GFP sorted cells per cluster was calculated as was the percentage of payload expressing genes per cluster as parallel measures of TTM-002 transduction.

For payload expressing cells, endothelial cells had the highest proportion of payload positive cells, followed by astrocytes (Table 33). For GFP+sorted cells, endothelial cells had the highest proportion of GFP positive cells, and astrocytes were the third highest cell type when sorting by proportion of cells expressing GFP (Table 33). These data indicate TTM-002 transduction exhibits an endothelial and astrocytic tropism. Furthermore, the astrocytic cluster had the second highest level of expression of Olig2 (oligodendrocytes demonstrated the greatest Olig2 expression). IHC staining was performed on brain samples isolated from AAV_TTM-002.GFP infected mice and demonstrated that GFP co-localized with some but not all Olig2+ cells. No co-staining was observed with mylein basic protein (MBP), a marker of oligodendrocytes. Co-staining with GFP was also not observed in NeuN positive cells (neurons), GFAP positive cells (astrocytes), and Iba1 positive cells (microglia). GFP staining was observed throughout the sagittal section of the mouse brain, which was demonstrative of increased staining in the midbrain. The GFP expressing cells observed did not have a bipolar morphology like oligodendrocyte progenitor (OPC) cells and therefore, together with the scRNA-Seq data, these results indicated that at day 28 post AAV treatment, Olig2+astrocytes in the midbrain are being transduced by AAV particles comprising a TTM-002 capsid, in a cell type specific tropism.

TABLE 33
Quantification of payload positive cells and GFP positive cells

| | % Payload | | % GFP |
|---|---|---|---|
| | Cells/ | | Cells/ |
| Cluster Identity | Cluster | Cluster Identity | Cluster |
| Endothelial-2 | 6.58 | Endothelial-2 | 6.58 |
| Astrocyte | 4.50 | Endothelial-1 | 3.45 |
| Pericytes | 4.23 | Vascular and | 2.38 |
| | | leptomeningeal | |
| | | Cells (VLM) | |
| Mature Oligos | 3.85 | Astrocyte | 2.37 |
| Endothelial-1 | 3.09 | Vascular smooth | 1.03 |
| | | muscle cells (VSC) | |
| Committed Oligos | 1.90 | Pericytes | 0.77 |
| Vascular smooth | 1.72 | Microglia | 0.00 |
| muscle cells (VSC) | | | |
| Microglia | 0.40 | Committed Oligos | 0.00 |
| Macrophages | 0.00 | Macrophages | 0.00 |
| Vascular and | 0.00 | Oligodendrocytes | 0.00 |
| leptomeningeal | | | |
| cells (VLM) | | | |
| Oligodendrocytes | 0.00 | Committed Oligos-2 | 0.00 |
| Committed Oligos-2 | 0.00 | Mature Oligos | 0.00 |

### Example 12. Dose Response Evaluation of the TTM-002 and TTM-027 AAV Capsid Variants

This Example investigates transduction of the TTM-002 (SEQ ID NO: 982 (amino acid) and SEQ ID NO: 984 (DNA), comprising SEQ ID NO: 2) and TTM-027 (SEQ ID NO: 69 (amino acid) and SEQ ID NO: 9 (DNA), comprising SEQ ID NO: 3272) capsid variants following intravenous administration at increasing doses in mice.

AAV particles were generated with the TTM-002 capsid variant or the TTM-027 capsid variant which comprised a single stranded viral genome encoding a histone protein with an HA tag (H3F3-HA) and a woodchuck hepatitis virus posttranscriptional regulatory element (WPRE) driven by a ubiquitous CBA promoter. The AAV particles comprising the TTM-002 capsid variant or the TTM-027 AAV capsid variant were administered to mice (n=3 mice per dosing group; Balb/c; 6-8 weeks of age) via tail vein injection at increasing doses of 1e12 vg/kg, 3.2e12 vg/kg, 1e13 vg/kg, 3.2e13 vg/kg, or 1e14 vg/kg. The dose of 3.2e12 vg/kg was approximately equivalent to the dose used per capsid in the cynomolgus macaques (Macaca fascicularis) in Example 7C above. The in-life period was 28 days and CNS tissues were collected for measuring transgene mRNA (expression) by qPCR and the percent of HA positive cells in the brain.

As shown in Table 37 and FIG. 10A, a dose dependent increase in transduction of both the TTM-002 and TTM-027 capsid variants was observed in the mouse cortex following intravenous administration of the AAV particles at the increasing doses The percent of cells transduced in the mouse cortex with TTM-027 was higher at all doses compared to TTM-002. At the highest dose tested, 1e14 vg/kg, TTM-027 transduced 65% of cells in the cortex, whereas TTM-002 transduced 38% of cells, with TTM-002 demonstrating an even distribution between neurons and astrocytes, identified by NeuN and Sox9 markers, respectively (Table 38). It was also observed that an increase in dose from 3.2e12 to 3.2e13 vg/kg resulted in a greater than 3 fold increase in percent positive cells. Consistent with the percentage of TTM-002 or TTM-027 positive cells, a dose dependent increase in transgene mRNA expression was also observed in the mouse brain following intravenous administration of the TTM-002 and TTM-027 capsid variants at the increasing doses (FIG. 10B and Table 37).

Taken together, these data demonstrate a dose-response across a 2-log dose range in mouse (1e12 to 1e14 VG/kg), without reaching saturation at the maximal dose (Table 37 and Table 38).

TABLE 37
Quantification of cells positive for the TTM-002 or TTM-027 capsid variant per mm²
(HA positive cells/mm²) or percentage of cells transduced with the TTM-002 or TTM-027 capsid
variants (% HA positive cells) in the mouse cortex measured by co-localization of chromogenic
HA staining and hematoxylin, as well as quantification by qPCR of transgene mRNA expression
relative to a housekeeping gene (mTBP) in the mouse brain following intravenous
administration (each value is the average of three individual measurements within each mouse
cortex isolated from three mice (n = 3))

| | | | | Transgene mRNA |
|---|---|---|---|---|
| Capsid | | HA Positive | % HA Positive | expression relative to |
| Variant | Dosage (vg/kg) | Cells/mm2 | Cells | housekeeping gene |
| TTM-002 | 1e14 | 679.147 | 37.668 | 9.3349 |
| | 3.2e13 | 414.043 | 26.279 | 3.7168 |
| | 1e13 | 277.497 | 19.078 | 2.8445 |
| | 3.2e12 | 151.841 | 8.450 | 0.9074 |
| | 1e12 | 61.018 | 3.616 | 0.1681 |
| Vehicle | 0 | 0.185 | 0.011 | 0.0003 |
| TTM-027 | 1e14 | 1019.777 | 65.451 | 19.9655 |
| | 3.2e13 | 835.525 | 46.076 | 14.0024 |
| | 1e13 | 585.873 | 31.717 | 6.7856 |
| | 3.2e 12 | 301.636 | 14.169 | 0.9269 |
| | 1e12 | 78.350 | 4.427 | 0.6379 |

TABLE 38
Quantification of total cells (HA positive cells/mm²), neurons (NeuN positive cells
that are also HA positive/mm²), and astrocytes (Sox9 positive cells that are also HA positive/mm²)
positive for the TTM-002 capsid variant per mm² or percentage of total cells (% HA
positive cells), neurons (% NeuN positive cells that are also HA positive), and astrocytes
(% Sox9 positive cells that are also HA positive) transduced with the TTM-002 capsid
variant in the mouse cortex by fluorescence microscopy and co-staining with HA and
cell-type specific markers (each value is the average of three individual measurements
within each mouse cortex isolated from three mice (n = 3))

| | | | | | % NeuN | | |
|---|---|---|---|---|---|---|---|
| | | | | NeuN | Positive | Sox9 | |
| | | | | Positive | Cells that | Positive | % Sox9 |
| | | HA | % HA | Cells that | are also | Cells that | Positive Cells |
| Capsid | Dosage | Positive | Positive | are also HA | HA | are also HA | that are also |
| Variant | (vg/kg) | Cells/mm² | Cells | positive/mm² | positive | positive/mm² | HA positive |
| TTM- | 1e14 | 495.9 | 28.6 | 154.7 | 13.5 | 26.2 | 12.8 |
| 002 | 3.2e13 | 344.0 | 18.8 | 126.8 | 9.2 | 19.8 | 14.6 |
| | 1e13 | 270.7 | 15.6 | 73.2 | 5.5 | 12.0 | 8.0 |
| | 3.2e12 | 176.9 | 8.2 | 59.2 | 3.6 | 8.5 | 4.4 |
| | 1e12 | 57.9 | 3.5 | 7.3 | 0.6 | 4.4 | 2.3 |
| Vehicle | 0 | 0 | 0 | 0 | 0 | 0 | 0 |

### Example 13: Individual Characterization of TTM-002 and TTM-001 Capsid Variants in Mice

The goal of these experiments was to determine the transduction level, tropism, ability to cross the blood brain barrier, and overall spatial distribution in the brain, heart, and liver of the TTM-001 and TTM-002 capsids and variants thereof relative to AAV9 following intravenous injection in mice. TTM-001 (SEQ ID NO: 981), TTM-002 (SEQ ID NO: 982), and variants of the TTM-002 and TTM-001 capsids comprising local modifications in loop IV were investigated including TTM-003 (SEQ ID NO: 36), TTM-006 (SEQ ID NO: 39), TTM-018 (SEQ ID NO: 51), and TTM-019 (SEQ ID NO: 52). The amino acid sequences for these capsid variants are provided, e.g., in Table 4.

AAV particles were generated with each of these capsid variants encapsulating a self-complementary genome encoding a fluorescent reporter construct, ZsGreen-HA, driven by a CAG promoter. Each capsid variant and AAV9 control were tested by intravenously administering by tail vein injection, the AAV particle formulation at 1e13 VG/kg to three BALB/c mice. The in-life period was 28 days and then various CNS and peripheral tissues were collected for measuring biodistribution and transgene mRNA expression.

The brains isolated from mice injected with the AAV particles encapsulated in the TTM-002 capsid, the TTM-001 capsid, or variants of the TTM-001 or TTM-002 capsid comprising local modifications were assayed to calculate ZsGreen expression and/or transgene DNA. Data were provided as fold over the AAV9 control (Table 39). All of the variants of the TTM-001 and TTM-002 capsids as well as TTM-001 and TTM-002 demonstrated increased CNS tropism and expression in the brain relative to AAV9 (Table 39; FIGS. 11A-11B). More specifically, TTM-001 and TTM-002 showed a broad distribution throughout the entire brain and spinal cord, outperforming the AAV9 control by approximately 30- and 40-fold, respectively, in terms of viral DNA biodistribution and transgene RNA expression (FIGS. 11A-11B; Table 39). The variants of the TTM-001 and TTM-002 capsids as well as TTM-001 and TTM-002 also demonstrated reduced mRNA and DNA expression in the liver by qPCR relative to the AAV9 control, with TTM-002 showing 14-fold lower gene expression than the AAV9 control (Table 39; FIGS. 11C-11D). Similar results were observed by immunohistochemistry (IHC) staining of the brain (including the cortex, thalamus, and cerebellum), spinal cord (grey matter), and liver post-transduction with AAV particles comprising the AAV capsid variants investigated. Transduction of the heart, skeletal muscle, and kidney did not show major differences between AAV9 and TTM-002.

TABLE 39
ZsGreen Expression and Transgene DNA and/or RNA expression for variants of the
TTM-001 and TTM-002 capsids relative to AAV9 in mice

| | Brain | | Liver | |
|---|---|---|---|---|
| | ZsGreen Expression | Transgene DNA | Transgene RNA | Transgene DNA fold |
| Capsid | fold change relative | fold change | fold change | change relative to |
| Variant | to AAV9 | relative to AAV9 | relative to AAV9 | AAV9 |
| AAV9 | 1.0 | 1.0 | 1.000 | 1.000 |
| TTM-001 | 32.3 | 31.9 | 0.337 | 0.348 |
| TTM-002 | 40.9 | 37.0 | 0.072 | 0.034 |
| TTM-006 | 16.1 | 36.9 | 0.036 | 0.013 |
| TTM-018 | 22.8 | 27.0 | 0.231 | 0.223 |
| TTM-019 | 14.6 | 14.4 | 0.117 | 0.112 |
| TTM-003 | 17.3 | 14.6 | 0.015 | 0.026 |

### Example 14: Evaluation of Vectorized siRNAs Targeting SOD1 In Vivo in NHPs

This Example investigates the ability of the modulatory polynucleotides VOYSOD1miR104-788.2 (SEQ ID NO: 2562 (DNA) or SEQ ID NO: 5013 (RNA)) or VOYSOD1miR127-860 (SEQ ID NO: 2579 (DNA) and SEQ ID NO: 4821 (RNA)) comprising a siRNA targeting SOD1 to reduce expression of SOD1 mRNA and/or protein in vivo in cynomolgus macaques (Macaca fascicularis).

AAV particles were generated that each comprised the TTM-002 capsid variant (SEQ ID NO: 982 (amino acid) and SEQ ID NO: 984 (DNA), comprising SEQ ID NO: 2), the TTM-003 capsid variant (SEQ ID NO: 36 (amino acid) and SEQ ID NO: 72 (DNA), comprising SEQ ID NO: 3589), or the TTM-027 capsid variant (SEQ ID NO: 69 (amino acid) and SEQ ID NO: 9 (DNA), comprising SEQ ID NO: 3272). These AAV particles each encapsulated a self-complementary viral genome encoding the VOYSOD1miR104-788.2 (SEQ ID NO: 5013 (RNA)) modulatory polynucleotide or the VOYSOD1miR127-860 (SEQ ID NO: 4821 (RNA)) modulatory polynucleotide under the control of the H1 promoter (SEQ ID NO: 83). The viral genome encoding the VOYSOD1miR104-788.2 modulatory polynucleotide comprised the nucleotide sequence of SEQ ID NO: 109; and the viral genome encoding the VOYSOD1miR127-860 modulatory polynucleotide comprised the nucleotide sequence of SEQ ID NO: 4820. These AAV particles were administered intravenously to male and female cynomolgus macaques (Macaca fascicularis; ~ 4 kg body weight; 2-5 years old; n=3) at a dose of 3e13 vg/kg, and three cynomolgus macaques (Macaca fascicularis; ~ 4 kg body weight; 2-5 years old; n=3) received a vehicle control. The in-life period was 56 days, and then CNS and peripheral tissues were collected for measuring SOD1 mRNA levels remaining post-treatment by RT-qPCR (cynomolgus SOD1 mRNA was normalized to the housekeeping gene of TBP, log transformed, and calibrated to the mean of the vehicle group); SOD1 protein levels; and viral DNA levels (biodistribution). The ventral horn tissues were acquired from punches taken from the C3 and L3 spinal cord.

As shown in Table 49, viral biodistribution of 2.5-7 viral genomes/diploid genome (vg/dg) was observed in both the cervical and lumbar ventral horn of the spinal cord at day 56 post-intravenous administration of all AAV particles comprising the different capsid and payload combinations, with the greatest biodistribution observed with AAV particles comprising either the TTM-027 capsid variant or the TTM-003 capsid variant. The strongest knockdown of SOD1 mRNA in the cervical and lumbar ventral horn of the spinal cord was observed following intravenous administration of AAV particles comprising the TTM-027 capsid variant or the TTM-003 capsid variant, encoding the VOYSOD1miR104-788.2 modulatory polynucleotide and comprising the viral genome sequence of SEQ ID NO: 109. More specifically, AAV particles comprising the TTM-027 capsid and comprising the viral genome of SEQ ID NO: 109 (encoding VOYSOD1miR104-788.2) resulted in a 55% knockdown of SOD1 mRNA in the lumbar ventral horn and a 41% knockdown of SOD1 mRNA in the cervical ventral horn in NHPs (Table 49). Little to no knockdown of SOD1 mRNA was observed in the spinal cord following administration of AAV particles encoding VOYSOD1miR127-860 and comprising the viral genome of SEQ ID NO: 4820, regardless of which capsid variant was used.

In the dorsal root ganglia (DRG), viral distribution of 1.6-8.8 vg/kg was observed at day 56 post-intravenous administration of AAV particles comprising the TTM-027 or TTM-002 capsid variant comprising the viral genome of SEQ ID NO: 109 (encoding the VOYSOD1miR104-788.2 modulatory polynucleotide) or the viral genome of SEQ ID NO: 4820 (encoding the VOYSOD1miR127-860 modulatory polynucleotide). Reduction of SOD1 mRNA was observed following administration with all AAV particles comprising the viral genome of SEQ ID NO: 109 (encoding the VOYSOD1miR104-788.2 modulatory polynucleotide) regardless of the capsid variant used, but the AAV particles comprising the TTM-027 capsid variant and the viral genome of SEQ ID NO: 109 demonstrated the strongest reduction in SOD1 mRNA levels. More specifically, the AAV particles comprising the TTM-027 capsid variant and the viral genome of SEQ ID NO: 109 (encoding the VOYSOD1miR104-788.2 modulatory polynucleotide) led to a 48% SOD1 mRNA reduction in the lumbar DRG, a 44% SOD1 mRNA reduction in the cervical DRG, and a 69% knockdown of SOD1 mRNA in the thoracic DRG. The AAV particles comprising the TTM-002 capsid variant and the viral genome of SEQ ID NO: 109 (encoding the VOYSOD1miR104-788.2 modulatory polynucleotide) yielded only 26-52% reduction in SOD1 mRNA in all levels of the DRG.

In the motor cortex, viral biodistribution of 4-10 vg/dg was observed at day 56 post-intravenous administration of all AAV particles comprising the different capsid and payload combinations, with the greatest biodistribution observed with AAV particles comprising the TTM-027 capsid variant. (Table 49). AAV particles comprising the TTM-027 capsid variant or the TTM-003 capsid variant both demonstrated increased biodistribution relative to TTM-002. Similar to the spinal cord, the strongest knockdown of SOD1 mRNA (Table 49) and decrease in SOD1 protein (FIG. 12A) in the motor cortex was also observed following intravenous administration of AAV particles comprising the TTM-027 capsid variant or the TTM-003 capsid variant, encoding the VOYSOD1miR104-788.2 modulatory polynucleotide and comprising the viral genome sequence of SEQ ID NO: 109. AAV particles comprising the TTM-027 capsid and comprising the viral genome of SEQ ID NO: 109 (encoding the VOYSOD1miR104-788.2) resulted in a 37% knockdown of SOD1 mRNA in the motor cortex. Additionally, biodistribution of viral mRNA from AAV particles comprising the TTM-027 capsid variant encapsulating the viral genome of SEQ ID NO: 109 (encodes the VOYSOD1miR104-788.2 modulatory polynucleotide) was detected in both the hypoglossal nucleus of the brain stem and in Betz cells in layer V of the motor cortex. However, little to no knockdown of SDO1 mRNA (Table 49) or decrease in SOD1 protein (FIG. 12B) was observed in the motor cortex following administration of AAV particles encoding VOYSOD1miR127-860 and comprising the viral genome of SEQ ID NO: 4820, regardless of which capsid variant was used.

Biodistribution and SOD1 mRNA knockdown was also measured in the peripheral tissues of the heart and the liver (Table 49). In the liver, viral biodistribution of 21-92 vg/dg was observed at day 56 post-intravenous administration of all AAV particles comprising the different capsid and payload combinations. AAV particles encoding the VOYSOD1miR104-788.2 modulatory polynucleotide and comprising the viral genome sequence of SEQ ID NO: 109, all resulted in comparable knockdown in the liver across all capsid variants tested; whereas little to no knockdown of SOD1 mRNA was observed in the liver with AAV particles encoding VOYSOD1miR127-860 and comprising the viral genome of SEQ ID NO: 4820. In the heart, viral biodistribution of 0.3-3.1 vg/dg was observed at day 56 post-intravenous administration of all AAV particles comprising the different capsid and payload combinations. Additionally, in the heart, AAV particles comprising the TTM-002 capsid variant and comprising the viral genome of SEQ ID NO: 109 (encoding VOYSOD1miR104-788.2) resulted in a 60% knockdown of SOD1 mRNA; AAV particles comprising the TTM-027 capsid variant and comprising the viral genome of SEQ ID NO: 109 (encoding VOYSOD1miR104-788.2) resulted in a 37% knockdown of SOD1 mRNA; and AAV particles comprising the TTM-003 capsid variant and comprising the viral genome of SEQ ID NO: 109 (encoding VOYSOD1miR104-788.2) resulted in a 40% knockdown of SOD1 mRNA. Similar to the liver, little to no knockdown of SOD1 mRNA was observed in the heart with AAV particles encoding VOYSOD1miR127-860 and comprising the viral genome of SEQ ID NO: 4820.

TABLE 49
Quantification of biodistribution (viral genome per diploid genomes (vg/dg)) and SOD1
mRNA levels remaining relative to the vehicle control at day 56 post-treatment with the
indicated AAV particles or vehicle control in the CNS and peripheral tissues of NHPs

| | | | | | SOD mRNA | |
|---|---|---|---|---|---|---|
| | | | | | Remaining | |
| | | | | | (normalized | |
| | | | Viral | | to TBP and | % SOD1 |
| | | Modulatory | Genome | | relative to | mRNA |
| Tissue | Capsid | Polynucleotide | Sequence | vg/dg | vehicle) | Knockdown |
| Cervical | Vehicle (1) | Vehicle (1) | Vehicle (1) | 0.0033 | 1.0 | N/A |
| Ventral | Vehicle (2) | Vehicle (2) | Vehicle (2) | 0.1 | 1.0 | N/A |
| Horn | TTM-002 | VOYSOD1miR104-788.2 | SEQ ID | 3.3 | 0.99 | 1 |
| | TTM-027 | (SEQ ID NO: 2562) | NO: 109 | 6.8 | 0.59 | 41 |
| | TTM-003 | | | 2.5 | 0.42 | 58 |
| | TTM-002 | VOYSOD1miR127-860 | SEQ ID | 4.1 | 1.2 | −20 |
| | TTM-027 | (SEQ ID NO: 2579) | NO: 4820 | 4.6 | 1.14 | −14 |
| | TTM-003 | | | 5.5 | 0.78 | 22 |
| Lumbar | Vehicle (1) | Vehicle (1) | Vehicle (1) | 0.0067 | 1.0 | N/A |
| Ventral | Vehicle (2) | Vehicle (2) | Vehicle (2) | 0.07 | 1.0 | N/A |
| Horn | TTM-002 | VOYSOD1miR104-788.2 | SEQ ID | 3.2 | 0.73 | 27 |
| | TTM-027 | (SEQ ID NO: 2562) | NO: 109 | 6.1 | 0.45 | 55 |
| | TTM-003 | | | 4.1 | 0.37 | 63 |
| | TTM-002 | VOYSOD1miR127-860 | SEQ ID | 5.0 | 1.03 | −3 |
| | TTM-027 | (SEQ ID NO: 2579) | NO: 4820 | 4.0 | 0.93 | 7 |
| | TTM-003 | | | 5.9 | 0.77 | 23 |
| Motor | Vehicle (1) | Vehicle (1) | Vehicle (1) | 0 | 1.0 | N/A |
| Cortex | Vehicle (2) | Vehicle (2) | Vehicle (2) | 0.04 | 1.0 | N/A |
| | TTM-002 | VOYSOD1miR104-788.2 | SEQ ID | 4.0 | 1.04 | −4 |
| | TTM-027 | (SEQ ID NO: 2562) | NO: 109 | 10.4 | 0.63 | 37 |
| | TTM-003 | | | 5.1 | 0.56 | 44 |
| | TTM-002 | VOYSOD1miR127-860 | SEQ ID | 5.3 | 0.99 | 1 |
| | TTM-027 | (SEQ ID NO: 2579) | NO: 4820 | 8.3 | 1.15 | −15 |
| | TTM-003 | | | 6.9 | 0.82 | 18 |
| Liver | Vehicle (1) | Vehicle (1) | Vehicle (1) | 0 | 1.0 | N/A |
| | Vehicle (2) | Vehicle (2) | Vehicle (2) | 0.3 | 1.0 | N/A |
| | TTM-002 | VOYSOD1miR104-788.2 | SEQ ID | 46.7 | 0.16 | 84 |
| | TTM-027 | (SEQ ID NO: 2562) | NO: 109 | 45.2 | 0.17 | 83 |
| | TTM-003 | | | 29.3 | 0.22 | 78 |
| | TTM-002 | VOYSOD1miR127-860 | SEQ ID | 91.8 | 0.87 | 13 |
| | TTM-027 | (SEQ ID NO: 2579) | NO: 4820 | 21.2 | 0.93 | 7 |
| | TTM-003 | | | 31.0 | 0.86 | 14 |
| Heart | Vehicle (1) | Vehicle (1) | Vehicle (1) | 0.01 | 1.0 | N/A |
| | Vehicle (2) | Vehicle (2) | Vehicle (2) | 0.08 | 1.0 | N/A |
| | TTM-002 | VOYSOD1miR104-788.2 | SEQ ID | 0.84 | 0.4 | 60 |
| | TTM-027 | (SEQ ID NO: 2562) | NO: 109 | 0.29 | 0.63 | 37 |
| | TTM-003 | | | 2.48 | 0.60 | 40 |
| | TTM-002 | VOYSOD1miR127-860 | SEQ ID | 0.46 | 1.02 | −2 |
| | TTM-027 | (SEQ ID NO: 2579) | NO: 4820 | 0.79 | 1.13 | −13 |
| | TTM-003 | | | 3.10 | 0.99 | 1 |
| Cervical | Vehicle (1) | Vehicle (1) | Vehicle (1) | 0.0033 | 1.00 | N/A |
| DRG | TTM-002 | VOYSOD1miR104-788.2 | SEQ ID | 1.68 | 0.73 | 27 |
| | TTM-027 | (SEQ ID NO: 2562) | NO: 109 | 2.56 | 0.56 | 44 |
| | TTM-002 | VOYSOD1miR127-860 | SEQ ID | 1.57 | 1.08 | −8 |
| | TTM-027 | (SEQ ID NO: 2579) | NO: 4820 | 1.89 | 1.29 | −29 |
| Thoracic | Vehicle (1) | Vehicle (1) | Vehicle (1) | 1.18 | 1.00 | N/A |
| DRG | TTM-002 | VOYSOD1miR104-788.2 | SEQ ID | 4.10 | 0.48 | 52 |
| | TTM-027 | (SEQ ID NO: 2562) | NO: 109 | 4.59 | 0.31 | 69 |
| | TTM-002 | VOYSOD1miR127-860 | SEQ ID | 8.83 | 0.81 | 19 |
| | TTM-027 | (SEQ ID NO: 2579) | NO: 4820 | 3.46 | 0.88 | 12 |
| Lumbar | Vehicle (1) | Vehicle (1) | Vehicle (1) | 0.0033 | 1.00 | N/A |
| DRG | TTM-002 | VOYSOD1miR104-788.2 | SEQ ID | 1.96 | 0.74 | 26 |
| | TTM-027 | (SEQ ID NO: 2562) | NO: 109 | 2.76 | 0.52 | 48 |
| | TTM-002 | VOYSOD1miR127-860 | SEQ ID | 1.88 | 1.14 | −14 |
| | TTM-027 | (SEQ ID NO: 2579) | NO: 4820 | 2.05 | 1.41 | −41 |

As provided in FIG. 13, the levels of SOD1 protein in the cerebral spinal fluid (CSF) was measured by ELISA as percent of total protein pre-dose on days 0, 29, 43, and 56 post-intravenous administration of the vehicles or AAV particles comprising the TTM-027 or TTM-003 capsid variant encapsulating the viral genome of SEQ ID NO: 109 (encodes the VOYSOD1miR104-788.2 modulatory polynucleotide). The AAV particles comprising the TTM-027 or TTM-003 capsid variant encapsulating the viral genome of SEQ ID NO: 109 (encodes the VOYSOD1miR104-788.2 modulatory polynucleotide) showed a trend in CSF SOD1 protein reduction. The protein levels shown in FIG. 13 are in line with the half-life of SOD1 protein (25.0+7.4 days) and presumed SOD1 pri-miRNA expression in a 56-day study.

Laser capture was used to collect motor neurons from the ventral horn to further measure SOD mRNA levels and knockdown, as laser capture microdissection allows for enrichment of motor neuron pools. The amount of SOD1 mRNA remaining normalized to a housekeeping gene (TBP) relative to the vehicle control and the percent SOD1 knockdown in these motor neurons post-treatment with the vehicle controls or AAV particles comprising either the TTM-027 or TTM-003 capsid variant encapsulating the viral genome of SEQ ID NO: 109 (encodes the VOYSOD1miR104-788.2 modulatory polynucleotide) is provided in Table 50. AAV particles comprising either the TTM-027 or TTM-003 capsid variant encapsulating the viral genome of SEQ ID NO: 109 (encodes the VOYSOD1miR104-788.2 modulatory polynucleotide) led to over 70-80% knockdown of SOD1 mRNA in motor neurons in the spinal cord.

TABLE 50
Quantification of SOD1 mRNA levels remaining relative to the vehicle control and
percent SOD1 knockdown in motor neurons isolated by laser capture at day 56 post-treatment
with the indicated AAV particles or vehicle control in the cervical or lumbar ventral horn of the
spinal cord of NHPs

| | | | | SOD mRNA | |
|---|---|---|---|---|---|
| | | | | Remaining | |
| | | | | (normalized to | |
| | | | Viral | TBP and | % SOD1 |
| | | Modulatory | Genome | relative to | mRNA |
| Tissue | Capsid | Polynucleotide | Sequence | vehicle) | Knockdown |
| Cervical | Vehicle (1) | Vehicle (1) | Vehicle (1) | 1.0 | N/A |
| Ventral | Vehicle (2) | Vehicle (2) | Vehicle (2) | 1.0 | N/A |
| Horn | TTM-027 | VOYSOD1miR104-788.2 | SEQ ID | 0.27 | 73 |
| | TTM-003 | (SEQ ID NO: 2562) | NO: 109 | 0.15 | 85 |
| Lumbar | Vehicle (1) | Vehicle (1) | Vehicle (1) | 1.0 | N/A |
| Ventral | Vehicle (2) | Vehicle (2) | Vehicle (2) | 1.0 | N/A |
| Horn | TTM-027 | VOYSOD1miR104-788.2 | SEQ ID | 0.18 | 82 |
| | TTM-003 | (SEQ ID NO: 2562) | NO: 109 | 0.17 | 83 |

The cellular tropism of the AAV particles comprising the TTM-002 capsid variant or the TTM-027 capsid variant encapsulating the viral genome of SEQ ID NO: 109 (encodes the VOYSOD1miR104-788.2 modulatory polynucleotide) was also quantified by measuring the copies of SOD1 mRNA copies/cell normalized to vehicle in the cervical, thoracic and lumbar regions of the spinal cord. Reduction of SOD1 mRNA copies in the cell relative to the vehicle was indicative of payload activity. Table 51 summarizes the cellular and tissue tropism of the AAV particles comprising the TTM-002 capsid variant or the TTM-027 capsid variant encapsulating the viral genome of SEQ ID NO: 109 (encodes the VOYSOD1miR104-788.2 modulatory polynucleotide as well as their activities. The AAV particles comprising the TTM-027 capsid variant encapsulating the viral genome of SEQ ID NO: 109 (encodes the VOYSOD1miR104-788.2 modulatory polynucleotide) demonstrated strong SOD1 knockdown in the spinal cord and a tropism profile that is favorable for treating a disease such as Amyotrophic Lateral Sclerosis (ALS).

TABLE 51
Summary of cellular and tissue tropism in the spinal cord and brain on day
56 post-intravenous administration of the AAV particles comprising the indicated
capsid + viral genome combination) or the vehicle control in NHPs

| | | | | | | SOD | |
|---|---|---|---|---|---|---|---|
| | | | | | Brainstem | mRNA | |
| | Viral | | Motor | | and Motor | copies/cell | SOD1 |
| | Genome | | Neron | Astrocyte | Cortex | (normalized | mRNA % |
| Capsid | Sequence | Tissue | Tropism | Tropism | Delivery | to vehicle) | reduction |
| Vehicle | Vehicle | Cervical | N/A | N/A | N/A | 100 | 0 |
| (1) | (1) | Thoracic | N/A | N/A | N/A | 100 | 0 |
| | | Lumbar | N/A | N/A | N/A | 100 | 0 |
| TTM- | SEQ ID | Cervical | Yes | Yes | + | 75.5 ± 28.0 | 24.5 ± 28.0 |
| 002 | NO: 109 | Thoracic | Yes | Yes | + | 58.1 ± 19.0 | 41.9 ± 19.0 |
| | | Lumbar | Yes | Yes | + | 65.5 ± 25.2 | 34.5 ± 25.2 |
| TTM- | SEQ ID | Cervical | Yes | Yes | ++ | 36.0 ± 7.5 | 64.0 ± 6.1 |
| 027 | NO: 109 | Thoracic | Yes | Yes | ++ | 66.7 ± 29.7 | 33.3 ± 24.2 |
| | | Lumbar | Yes | Yes | ++ | 36.9 ± 5.3 | 63.1 ± 4.3 |

Small RNA processing analysis via high throughput small RNA deep sequencing to measure guide to passenger ratio, abundance of guide and passenger strands relative to the total endogenous pool of miRNAs (saturation level of the endogenous miRNA pathway), and precision of processing at the 5′-end of the guide strand was also performed on RNA isolated from the cervical spinal cords of the NHPs on day 56 post-intravenous treatment with the AAV particles comprising the TTM-002 capsid variant or the TTM-027 capsid variant encoding the VOYSOD1miR104-788.2 modulatory polynucleotide or the VOYSOD1miR127-860 modulatory polynucleotide. Table 52 shows the level of mature exogenous SOD1 targeting polynucleotides (guide or antisense strand level) relative to the total endogenous pool of miRNAs (guide: endogenous miRNA ratio %) to ensure a lack of overharnessing of the endogenous miRNA biogenesis pathway, where a lower percentage is more favorable to prevent any effects related to endogenous miRNA pathway saturation. The guide to passenger strand level is also provided in Table 52. A higher guide to passenger strand ratio is more favorable for potency and selectivity and to minimize any passenger strand effects. Table 52 also shows the 5′ end precision processing of the modulatory polynucleotides evaluated. The 5′ end processing of the modulatory polynucleotides indicates that the constructs are being processed correctly at the 5′ end and a higher value is more favorable. As the seed region is used for recognition of the target mRNA and starts at a fixed position relative to the 5′ end of the guide strand, accurate processing of the 5′ end of the guide strand is needed. Both the viral genome of SEQ ID NO: 4820 encoding the VOYSOD1miR127-860 modulatory polynucleotide and the viral genome of SEQ ID NO: 109 encoding the VOYSOD1miR104-788.2 modulatory polynucleotide exhibited low artificial miRNA expression in the desired range (Table 52) when encapsulated in AAV particles comprising either the TTM-002 capsid variant or the TTM-027 capsid variant. The guide to passenger strand ratio and the 5′ precision processing were also favorable for the viral genome of SEQ ID NO: 109 encoding the VOYSOD1miR104-788.2 modulatory polynucleotide when encapsulated in AAV particles comprising the TTM-027 capsid variant. The levels of the endogenous microRNA, mir-122a was measured in NHPs that received the vehicle control or AAV particles comprising the TTM-027 capsid variant, encapsulating the viral genome of SEQ ID NO: 109 encoding the VOYSOD1miR104-788.2 modulatory polynucleotide, and the levels were equivalent, indicating that endogenous miRNA expression did not appear to be disrupted.

TABLE 52
Guide to passenger ratio, abundance of guide strand relative to the total endogenous
pool of miRNAs guide: endog. miRNA ratio), and precision of processing at the 5′-end of the
guide strand in the cervical spinal cord for the VOYSOD1miR104-788.2 modulatory
polynucleotide or the VOYSOD1miR127-860 modulatory polynucleotide delivered by AAV
particles comprising the TTM-002 or TTM-027 capsid variant at day 56 post-intravenous
administration (each value is an average of samples taken from three NHPs)

| | Viral | | | Guide: | Guide/ | |
|---|---|---|---|---|---|---|
| | Genome | Modulatory | | Endogenous | Passenger | 5′ Precision |
| Capsid | Sequence | Polynucleotide | Tissue | miRNA ratio (% ) | Ratio | Processing |
| TTM-002 | SEQ ID | VOYSOD1miR | Cervical | 0.01 | 24.46 | 83.04 |
| | NO: 4820 | 127-860 | Spinal | | | |
| | SEQ ID | VOYSOD1miR | Cord | 0.59 | 202.34 | 95.80 |
| | NO: 109 | 104-788.2 | (ventral | | | |
| TTM-027 | SEQ ID | VOYSOD1miR | horn) | 0.02 | 51.48 | 74.58 |
| | NO: 4820 | 127-860 | | | | |
| | SEQ ID | VOYSOD1miR | | 5.26 | 103.48 | 92.93 |
| | NO: 109 | 104-788.2 | | | | |

Taken together, these data indicate that the VOYSOD1miR104-788.2 modulatory polynucleotide was capable of reducing SOD1 mRNA expression in vivo and administration of this modulatory polynucleotides by AAV particles comprising the capsid variants described herein (e.g., TTM-027, TTM-002, and TTM-003) resulted in increased and suitable delivery to the brain and spinal cord of NHPs, with neuronal and astrocytic tropism observed.

### XI. Equivalents and Scope

The disclosures of each and every patent, patent application, and publication cited herein are hereby incorporated herein by reference in their entirety. While this invention has been disclosed with reference to certain embodiments, it is apparent that further embodiments and variations of this invention may be devised by others skilled in the art without departing from the true spirit and scope of the invention. The appended claims are intended to be construed to include all such embodiments and equivalent variations.

We claim:

1. An AAV particle comprising an AAV capsid variant and a nucleic acid encoding a polynucleotide that targets human SOD1, wherein the AAV capsid variant comprises an amino acid sequence having the following formula: [N1]-[N2]-[N3], wherein:
(i) [N1] comprises X1, X2, and X3, wherein at least one of X1, X2, or X3 is G;
(ii) [N2] comprises the amino acid sequence of SPH; and
(iii) [N3] comprises X4, X5, and X6, wherein at least one of X4, X5, or X6 is a basic amino acid, e.g., a K or R;
wherein [N1]-[N2]-[N3] is present in hypervariable loop IV; and
wherein the AAV capsid variant comprises an amino acid sequence at least 95% identical to the amino acid sequence of positions 203-736 of SEQ ID NO: 138.

2. The AAV particle of claim 1, wherein [N3] comprises SKA, KSG, ARM, VKS, ASR, VKI, KKN, VRM, RKA, KTS, KFG, KIG, KLG, KTT, KTY, KYG, SKD, SKP, TRG, VRG, KRG, GAR, KSA, KSR, SKL, SRA, SKR, SLR, SRG, SSR, FLR, SKW, SKS, WKA, VRR, SKV, SKT, SKG, GKA, TKA, NKA, SKL, SKN, AKA, KTG, KSL, KSE, KSV, KSW, KSN, KHG, KSQ, KSK, KLW, WKG, KMG, KMA, or RSG.

3. The AAV particle of claim 1 or 2, wherein [N2]-[N3] comprises SPHSKA (SEQ ID NO: 941), SPHKSG (SEQ ID NO: 946), SPHARM (SEQ ID NO: 947), SPHVKS (SEQ ID NO: 948), SPHASR (SEQ ID NO: 949), SPHVKI (SEQ ID NO: 950), SPHKKN (SEQ ID NO: 954), SPHVRM (SEQ ID NO: 955), SPHRKA (SEQ ID NO: 956), SPHKFG (SEQ ID NO: 957), SPHKIG (SEQ ID NO: 958), SPHKLG (SEQ ID NO: 959), SPHKTS (SEQ ID NO: 963), SPHKTT (SEQ ID NO: 964), SPHKTY (SEQ ID NO: 965), SPHKYG (SEQ ID NO: 966), SPHSKD (SEQ ID NO: 967), SPHSKP (SEQ ID NO: 968, SPHTRG (SEQ ID NO: 972), SPHVRG (SEQ ID NO: 973), SPHKRG (SEQ ID NO: 974), SPHGAR (SEQ ID NO: 975), SPHKSA (SEQ ID NO: 977), SPHKSR (SEQ ID NO: 951), SPHSKL (SEQ ID NO: 960), SPHSRA (SEQ ID NO: 969), SPHSKR (SEQ ID NO: 978), SPHSLR (SEQ ID NO: 952, SPHSRG (SEQ ID NO: 961), SPHSSR (SEQ ID NO: 970), SPHFLR (SEQ ID NO: 979), SPHSKW (SEQ ID NO: 953), SPHSKS (SEQ ID NO: 1402), SPHWKA (SEQ ID NO: 971), SPHVRR (SEQ ID NO: 980), SPHSKT (SEQ ID NO: 1403), SPHSKG (SEQ ID NO: 1404), SPHGKA (SEQ ID NO: 1405), SPHNKA (SEQ ID NO: 1406), SPHSKN (SEQ ID NO: 1407), SPHAKA (SEQ ID NO: 1408), SPHSKV (SEQ ID NO: 1409), SPHKTG (SEQ ID NO: 1410), SPHTKA (SEQ ID NO: 1411), SPHKSL (SEQ ID NO: 1412), SPHKSE (SEQ ID NO: 1413), SPHKSV (SEQ ID NO: 1414), SPHKSW (SEQ ID NO: 1415), SPHKSN (SEQ ID NO: 1416), SPHKHG (SEQ ID NO: 1417), SPHKSQ (SEQ ID NO: 1418), SPHKSK (SEQ ID NO: 1419), SPHKLW (SEQ ID NO: 1420), SPHWKG (SEQ ID NO: 1421), SPHKMG (SEQ ID NO: 1422), SPHKMA (SEQ ID NO: 1423), or SPHRSG (SEQ ID NO: 976).

4. The AAV particle of any one of claims 1-3, wherein [N1] comprises GSG, GHD, VSG, GQD, CSG, GRG, CSH, GQS, GSH, RVG, GSC, GLL, GDD, GHE, GNY, MSG, RNG, TSG, ISG, GPG, ESG, SSG, GNG, ASG, NSG, LSG, GGG, KSG, HSG, GTG, PSG, GSV, RSG, GIG, WSG, DSG, IDG, GLG, DAG, DGG, MEG, ENG, GSA, KNG, KEG, AIG, GYD, GHG, GRD, GND, GPD, GMG, GQV, GHN, GHP, or GHS.

5. The AAV particle of any one of claims 1-4, wherein [N1]-[N2]-[N3] comprises:
(i)
(SEQ ID NO: 1369)
GSGSPHSKA,
(SEQ ID NO: 1370)
GHDSPHKSG,
(SEQ ID NO: 1586)
VSGSPHSKA,
(SEQ ID NO: 1579)
GSGSPHARM,
(SEQ ID NO: 1580)
GSGSPHVKS,
(SEQ ID NO: 1581)
GQDSPHKSG,
(SEQ ID NO: 1582)
GSGSPHASR,
(SEQ ID NO: 1583)
GSGSPHVKI,
(SEQ ID NO: 1584)
GSGSPHKKN,
(SEQ ID NO: 1585)
GSGSPHVRM,
(SEQ ID NO: 1587)
CSGSPHSKA,
(SEQ ID NO: 1588)
GSGSPHRKA,
(SEQ ID NO: 1589)
CSGSPHKTS,
(SEQ ID NO: 1590)
CSHSPHKSG,
(SEQ ID NO: 1591)
GQSSPHRSG,
(SEQ ID NO: 1592)
GRGSPHASR,
(SEQ ID NO: 1593)
GRGSPHSKA,
(SEQ ID NO: 1594)
GSGSPHKFG,
(SEQ ID NO: 1595)
GSGSPHKIG,
(SEQ ID NO: 1596)
GSGSPHKLG,
(SEQ ID NO: 1597)
GSGSPHKTS,
(SEQ ID NO: 1598)
GSGSPHKTT,
(SEQ ID NO: 1599)
GSGSPHKTY,
(SEQ ID NO: 1600)
GSGSPHKYG,
(SEQ ID NO: 1601)
GSGSPHSKD,
(SEQ ID NO: 1602)
GSGSPHSKP,
(SEQ ID NO: 1603)
GSGSPHTRG,
(SEQ ID NO: 1604)
GSGSPHVRG,
(SEQ ID NO: 1605)
GSHSPHKRG,
(SEQ ID NO: 1606)
GSHSPHKSG,
(SEQ ID NO: 1607)
VSGSPHASR,
(SEQ ID NO: 1608)
VSGSPHGAR,
(SEQ ID NO: 1609)
VSGSPHKFG,
(SEQ ID NO: 1610)
GHDSPHKRG,
(SEQ ID NO: 1611)
GDDSPHKSG,
(SEQ ID NO: 1612)
GHESPHKSA,
(SEQ ID NO: 1613)
GHDSPHKSA,
(SEQ ID NO: 1614)
GNYSPHKIG,
(SEQ ID NO: 1615)
GHDSPHKSR,
(SEQ ID NO: 1616)
GSGSPHSKL,
(SEQ ID NO: 1617)
GSGSPHSRA,
(SEQ ID NO: 1618)
GSGSPHSKR,
(SEQ ID NO: 1619)
GSGSPHSLR,
(SEQ ID NO: 1620)
GSGSPHSRG,
(SEQ ID NO: 1621)
GSGSPHSSR,
(SEQ ID NO: 1622)
RVGSPHSKA,
(SEQ ID NO: 1623)
GSCSPHRKA,
(SEQ ID NO: 1624)
GSGSPHFLR,
(SEQ ID NO: 1625)
GSGSPHSKW,
(SEQ ID NO: 1626)
GSGSPHSKS,
(SEQ ID NO: 1627)
GLLSPHWKA,
(SEQ ID NO: 1628)
GSGSPHVRR,
(SEQ ID NO: 1629)
GSGSPHSKV,
(SEQ ID NO: 1630)
MSGSPHSKA,
(SEQ ID NO: 1631)
RNGSPHSKA,
(SEQ ID NO: 1632)
TSGSPHSKA,
(SEQ ID NO: 1633)
ISGSPHSKA,
(SEQ ID NO: 1634)
GPGSPHSKA,
(SEQ ID NO: 1635)
GSGSPHSKT,
(SEQ ID NO: 1636)
ESGSPHSKA,
(SEQ ID NO: 1637)
SSGSPHSKA,
(SEQ ID NO: 1638)
GNGSPHSKA,
(SEQ ID NO: 1639)
ASGSPHSKA,
(SEQ ID NO: 1640)
NSGSPHSKA,
(SEQ ID NO: 1641)
LSGSPHSKA,
(SEQ ID NO: 1642)
GGGSPHSKA,
(SEQ ID NO: 1643)
KSGSPHSKA,
(SEQ ID NO: 1644)
GGGSPHSKS,
(SEQ ID NO: 1645)
GSGSPHSKG,
(SEQ ID NO: 1646)
HSGSPHSKA,
(SEQ ID NO: 1647)
GTGSPHSKA,
(SEQ ID NO: 1648)
PSGSPHSKA,
(SEQ ID NO: 1649)
GSVSPHGKA,
(SEQ ID NO: 1650)
RSGSPHSKA,
(SEQ ID NO: 1651)
GSGSPHTKA,
(SEQ ID NO: 1652)
GIGSPHSKA,
(SEQ ID NO: 1653)
WSGSPHSKA,
(SEQ ID NO: 1654)
DSGSPHSKA,
(SEQ ID NO: 1655)
IDGSPHSKA,
(SEQ ID NO: 1656)
GSGSPHNKA,
(SEQ ID NO: 1657)
GLGSPHSKS,
(SEQ ID NO: 1658)
DAGSPHSKA,
(SEQ ID NO: 1659)
DGGSPHSKA,
(SEQ ID NO: 1660)
MEGSPHSKA,
(SEQ ID NO: 1661)
ENGSPHSKA,
(SEQ ID NO: 1662)
GSASPHSKA,
(SEQ ID NO: 1663)
GNGSPHSKS,
(SEQ ID NO: 1664)
KNGSPHSKA,
(SEQ ID NO: 1665)
KEGSPHSKA,
(SEQ ID NO: 1666)
AIGSPHSKA,
(SEQ ID NO: 1667)
GSGSPHSKN,
(SEQ ID NO: 1668)
GSGSPHAKA,
(SEQ ID NO: 1669)
GHDSPHKIG,
(SEQ ID NO: 1670)
GYDSPHKSG,
(SEQ ID NO: 1671)
GHESPHKSG,
(SEQ ID NO: 1672)
GHDSPHKTG,
(SEQ ID NO: 1673)
GRGSPHKRG,
(SEQ ID NO: 1581)
GQDSPHKSG,
(SEQ ID NO: 1674)
GHDSPHKSL,
(SEQ ID NO: 1675)
GHGSPHSKA,
(SEQ ID NO: 1676)
GHDSPHKSE,
(SEQ ID NO: 1586)
VSGSPHSKA,
(SEQ ID NO: 1677)
GRDSPHKSG,
(SEQ ID NO: 1678)
GNDSPHKSV,
(SEQ ID NO: 1679)
GQDSPHKIG,
(SEQ ID NO: 1680)
GHDSPHKSV,
(SEQ ID NO: 1681)
GPDSPHKIG,
(SEQ ID NO: 1682)
GPDSPHKSG,
(SEQ ID NO: 1683)
GHDSPHKSW,
(SEQ ID NO: 1684)
GHDSPHKSN,
(SEQ ID NO: 1685)
GMGSPHSKT,
(SEQ ID NO: 1686)
GHDSPHKHG,
(SEQ ID NO: 1687)
GQVSPHKSG,
(SEQ ID NO: 1688)
GDDSPHKSV,
(SEQ ID NO: 1689)
GHNSPHKSG,
(SEQ ID NO: 1690)
GNGSPHKRG,
(SEQ ID NO: 1691)
GHDSPHKYG,
(SEQ ID NO: 1692)
GHDSPHKSQ,
(SEQ ID NO: 1693)
GNDSPHKIG,
(SEQ ID NO: 1694)
GHDSPHKSK,
(SEQ ID NO: 1695)
GHDSPHKLW,
(SEQ ID NO: 1696)
GHPSPHWKG,
(SEQ ID NO: 1697)
GHDSPHKMG,
(SEQ ID NO: 1698)
GHDSPHKMA,
or
(SEQ ID NO: 1699)
GHSSPHRSG;
or
(ii)
(SEQ ID NO: 1369)
GSGSPHSKA,
(SEQ ID NO: 1370)
GHDSPHKSG,
or
(SEQ ID NO: 1586)
VSGSPHSKA.

6. The AAV particle of any one of claims 1-5, which further comprises:
(i) [N0], wherein [N0] comprises TIN, TEN, TER, SMN, TIM, YLS, GLS, MPE, MEG, MEY, AEW, CEW, ANN, IPE, ADM, IEY, ADY, IET, MEW, CEY, RIN, MEI, LEY, ADW, IEI, DIM, FEQ, MEF, CDQ, LPE, IEN, MES, AEI, VEY, IIN, TSN, IEV, MEM, AEV, MDA, VEW, AEQ, LEW, MEL, MET, MEA, IES, MEV, CEI, ATN, MDG, QEV, ADQ, NMN, IEM, ISN, TGN, QQQ, HDW, IEG, TII, TFP, TEK, EIN, TVN, TFN, SIN, TSY, ELH, AlN, SVN, TDN, TFH, TVH, TSS, TID, TCN, NIN, TEH, AEM, AIK, TDK, TFK, SDQ, TEI, NTN, TET, SIK, TEL, TEA, TAN, TIY, TFS, TES, TTN, TED, TNN, EVH, TIS, TVR, TDR, TIK, NHI, TIP, ESD, TDL, TVP, TVI, AEH, NCL, TVK, NAD, TIT, NCV, TIR, NAL, VIN, TIQ, TEF, TRE, QGE, SEK, NVN, GGE, EFV, SDK, TEQ, EVQ, TEY, NCW, TDV, SDI, NSI, NSL, EVV, TEP, SEL, TWQ, TEV, AVN, GVL, TLN, TEG, TRD, NAI, AEN, AET, ETA, or NNL; and/or
(ii) [N4], wherein [N4] comprises QNQQ (SEQ ID NO: 1701), WNQQ (SEQ ID NO: 1702), QYYV (SEQ ID NO: 1703), RRQQ (SEQ ID NO: 1704), GCGQ (SEQ ID NO: 1705), LRQQ (SEQ ID NO: 1706), RNQQ (SEQ ID NO: 1707), VNQQ (SEQ ID NO: 1708), FRLQ (SEQ ID NO: 1709), FNQQ (SEQ ID NO: 1710), LLQQ (SEQ ID NO: 1711), SNQQ (SEQ ID NO: 1712), RLQQ (SEQ ID NO: 1713), LNQQ (SEQ ID NO: 1714), QRKL (SEQ ID NO: 1715), LRRQ (SEQ ID NO: 1716), QRLR (SEQ ID NO: 1717), QRRL (SEQ ID NO: 1718), RRLQ (SEQ ID NO: 1719), RLRQ (SEQ ID NO: 1720), SKRQ (SEQ ID NO: 1721), QLYR (SEQ ID NO: 1722), QLTV (SEQ ID NO: 1723), QNKQ (SEQ ID NO: 1724), KNQQ (SEQ ID NO: 1725), QKQQ (SEQ ID NO: 1726), QTQQ (SEQ ID NO: 1727), QNHQ (SEQ ID NO: 1728), QHQQ (SEQ ID NO: 1729), QNQH (SEQ ID NO: 1730), QHRQ (SEQ ID NO: 1731), LTQQ (SEQ ID NO: 1732), QNQW (SEQ ID NO: 1733), QNTH (SEQ ID NO: 1734), RRRQ (SEQ ID NO: 1735), QYQQ (SEQ ID NO: 1736), QNDQ (SEQ ID NO: 1737), QNRH (SEQ ID NO: 1738), RDQQ (SEQ ID NO: 1739), PNLQ (SEQ ID NO: 1740), HVRQ (SEQ ID NO: 1741), PNQH (SEQ ID NO: 1742), HNQQ (SEQ ID NO: 1743), QSQQ (SEQ ID NO: 1744), QPAK (SEQ ID NO: 1745), QNLA (SEQ ID NO: 1746), QNQL (SEQ ID NO: 1747), QGQQ (SEQ ID NO: 1748), LNRQ (SEQ ID NO: 1749), QNPP (SEQ ID NO: 1750), QNLQ (SEQ ID NO: 1751), QDQE (SEQ ID NO: 1752), QDQQ (SEQ ID NO: 1753), HWQQ (SEQ ID NO: 1755), PNQQ (SEQ ID NO: 1756), PEQQ (SEQ ID NO: 1757), QRTM (SEQ ID NO: 1758), LHQH (SEQ ID NO: 1759), QHRI (SEQ ID NO: 1760), QYIH (SEQ ID NO: 1761), QKFE (SEQ ID NO: 1762), QFPS (SEQ ID NO: 1763), QNPL (SEQ ID NO: 1764), QAIK (SEQ ID NO: 1765), QNRQ (SEQ ID NO: 1766), QYQH (SEQ ID NO: 1767), QNPQ (SEQ ID NO: 1768), QHQL (SEQ ID NO: 1769), QSPP (SEQ ID NO: 1770), QAKL (SEQ ID NO: 1771), KSQQ (SEQ ID NO: 1772), QDRP (SEQ ID NO: 1773), QNLG (SEQ ID NO: 1774), QAFH (SEQ ID NO: 1775), QNAQ (SEQ ID NO: 1776), HNQL (SEQ ID NO: 1777), QKLN (SEQ ID NO: 1778), QNVQ (SEQ ID NO: 1779), QAQQ (SEQ ID NO: 1780), QTPP (SEQ ID NO: 1781), QPPA (SEQ ID NO: 1782), QERP (SEQ ID NO: 1783), QDLQ (SEQ ID NO: 1784), QAMH (SEQ ID NO: 1785), QHPS (SEQ ID NO: 1786), PGLQ (SEQ ID NO: 1787), QGIR (SEQ ID NO: 1788), QAPA (SEQ ID NO: 1789), QIPP (SEQ ID NO: 1790), QTQL (SEQ ID NO: 1791), QAPS (SEQ ID NO: 1792), QNTY (SEQ ID NO: 1793), QDKQ (SEQ ID NO: 1794), QNHL (SEQ ID NO: 1795), QIGM (SEQ ID NO: 1796), LNKQ (SEQ ID NO: 1797), PNQL (SEQ ID NO: 1798), QLQQ (SEQ ID NO: 1799), QRMS (SEQ ID NO: 2897), QGIL (SEQ ID NO: 2907), QDRQ (SEQ ID NO: 2917), RDWQ (SEQ ID NO: 3081), QERS (SEQ ID NO: 3238), QNYQ (SEQ ID NO: 3590), QRTC (SEQ ID NO: 3849), QIGH (SEQ ID NO: 3850), QGAI (SEQ ID NO: 3851), QVPP (SEQ ID NO: 3852), QVQQ (SEQ ID NO: 3853), LMRQ (SEQ ID NO: 3854), QYSV (SEQ ID NO: 3855), QAIT (SEQ ID NO: 3856), QKTL (SEQ ID NO: 3857), QLHH (SEQ ID NO: 3858), QNII (SEQ ID NO: 3859), QGHH (SEQ ID NO: 3860), QSKV (SEQ ID NO: 3861), QLPS (SEQ ID NO: 3862), IGKQ (SEQ ID NO: 3863), QAIH (SEQ ID NO: 3864), QHGL (SEQ ID NO: 3865), QFMC (SEQ ID NO: 3866), QNQM (SEQ ID NO: 3867), QHLQ (SEQ ID NO: 3868), QPAR (SEQ ID NO: 3869), QSLQ (SEQ ID NO: 3870), QSQL (SEQ ID NO: 3871), HSQQ (SEQ ID NO: 3872), QMPS (SEQ ID NO: 3873), QGSL (SEQ ID NO: 3874), QVPA (SEQ ID NO: 3875), HYQQ (SEQ ID NO: 3876), QVPS (SEQ ID NO: 3877), RGEQ (SEQ ID NO: 3878), PGQQ (SEQ ID NO: 3879), LEQQ (SEQ ID NO: 3880), QNQS (SEQ ID NO: 3881), QKVI (SEQ ID NO: 3882), QNND (SEQ ID NO: 3883), QSVH (SEQ ID NO: 3884), QPLG (SEQ ID NO: 3885), HNQE (SEQ ID NO: 3886), QIQQ (SEQ ID NO: 3887), QVRN (SEQ ID NO: 3888), PSNQ (SEQ ID NO: 3889), QVGH (SEQ ID NO: 3890), QRDI (SEQ ID NO: 3891), QMPN (SEQ ID NO: 3892), RGLQ (SEQ ID NO: 3893), PSLQ (SEQ ID NO: 3894), QRDQ (SEQ ID NO: 3895), QAKG (SEQ ID NO: 3896), QSAH (SEQ ID NO: 3897), QSTM (SEQ ID NO: 3898), QREM (SEQ ID NO: 3899), QYRA (SEQ ID NO: 3900), QRQQ (SEQ ID NO: 3901), QWQQ (SEQ ID NO: 3902), QRMN (SEQ ID NO: 3903), GDSQ (SEQ ID NO: 3904), QKIS (SEQ ID NO: 3905), PSMQ (SEQ ID NO: 3906), SPRQ (SEQ ID NO: 3907), MEQQ (SEQ ID NO: 3908), QYQN (SEQ ID NO: 3909), QIRQ (SEQ ID NO: 3910), QSVQ (SEQ ID NO: 3911), RSQQ (SEQ ID NO: 3912), QNKL (SEQ ID NO: 3913), QIQH (SEQ ID NO: 3914), PRQQ (SEQ ID NO: 3915), HTQQ (SEQ ID NO: 3916), QRQH (SEQ ID NO: 3917), RNQE (SEQ ID NO: 3918), QSKQ (SEQ ID NO: 3919), QNQP (SEQ ID NO: 3920), QSPQ (SEQ ID NO: 3921), QTRQ (SEQ ID NO: 3922), QNLH (SEQ ID NO: 3923), QNQE (SEQ ID NO: 3924), LNQP (SEQ ID NO: 3925), QNQD (SEQ ID NO: 3926), QNLL (SEQ ID NO: 3927), QLVI (SEQ ID NO: 3928), RTQE (SEQ ID NO: 3929), QTHQ (SEQ ID NO: 3930), QDQH (SEQ ID NO: 3931), QSQH (SEQ ID NO: 3932), VRQQ (SEQ ID NO: 3933), AWQQ (SEQ ID NO: 3934), QSVP (SEQ ID NO: 3935), QNIQ (SEQ ID NO: 3936), LDQQ (SEQ ID NO: 3937), PDQQ (SEQ ID NO: 3938), ESQQ (SEQ ID NO: 3939), QRQL (SEQ ID NO: 3940), QIIV (SEQ ID NO: 3941), QKQS (SEQ ID NO: 3942), QSHQ (SEQ ID NO: 3943), QFVV (SEQ ID NO: 3944), QSQP (SEQ ID NO: 3945), QNEQ (SEQ ID NO: 3946), INQQ (SEQ ID NO: 3947), RNRQ (SEQ ID NO: 3948), RDQK (SEQ ID NO: 3949), QWKR (SEQ ID NO: 3950), ENRQ (SEQ ID NO: 3951), QTQP (SEQ ID NO: 3952), QKQL (SEQ ID NO: 3953), RNQL (SEQ ID NO: 3954), ISIQ (SEQ ID NO: 3955), QTVC (SEQ ID NO: 3956), QQIM (SEQ ID NO: 3957), LNHQ (SEQ ID NO: 3958), QNQA (SEQ ID NO: 3959), QMIH (SEQ ID NO: 3960), RNHQ (SEQ ID NO: 3961), or QKMN (SEQ ID NO: 3962).

7. The AAV particle of claim 6, wherein [N0]-[N1]-[N2]-[N3]-[N4] comprises:
(i) the amino acid sequence of any one of SEQ ID NOs: 2242-2886; or
(ii) TENVSGSPHSKAQNQQ (SEQ ID NO: 2283), TERVSGSPHSKAQNQQ (SEQ ID NO: 2272), TINGSGSPHSKAQNQQ (SEQ ID NO: 2242), or TINGHDSPHKSGQNQQ (SEQ ID NO: 2243).

8. The AAV particle of claim 6 or 7, wherein:
(i) [N0] is present at amino acids 450-452, numbered according to SEQ ID NO: 981, 982, or 138;
(ii) [N1] is present at amino acids 453-455, numbered according to SEQ ID NO: 981, 982, or 138;
(iii) [N2] is present at amino acids 456-458, numbered according to SEQ ID NO: 981 or 982;
(iv) [N3] is present at amino acids 459-461, numbered according to SEQ ID NO: 981 or 982;
(v) [N4] is present at amino acids 462-465, numbered according to SEQ ID NO: 981 or 982; and/or
(vi) [N0]-[N1]-[N2]-[N3]-[N4] is present at amino acids 450-465, numbered according to SEQ ID NO: 981 or 982.

9. An AAV particle comprising an AAV capsid variant and a nucleic acid encoding a polynucleotide that targets human SOD1, wherein the AAV capsid variant comprises:
(i) the amino acid sequence of SPHSKA (SEQ ID NO: 941) in hypervariable loop IV; and
(ii) an amino acid sequence at least 95% identical to the amino acid sequence of positions 203-736 of SEQ ID NO: 138.

10. The AAV particle of claim 9, wherein the amino acid sequence of SPHSKA (SEQ ID NO: 941) is present immediately subsequent to amino acid 455, numbered according to SEQ ID NO: 981.

11. The AAV particle of claim 9 or 10, wherein the AAV capsid variant further comprises the amino acid E at position 451, numbered according to SEQ ID NO: 981.

12. The AAV particle of any one of claims 9-11, wherein the AAV capsid variant comprises the amino acid V at position 453, numbered according to SEQ ID NO: 981.

13. The AAV particle of any one of claims 9-12, wherein the AAV capsid variant comprises the amino acid R at position 452, numbered according to SEQ ID NO: 981.

14. The AAV particle of any one of claims 1-13, wherein the AAV capsid variant comprises:
(i) an amino acid sequence at least 95% or at least 98% identical to amino acids 203-742 of SEQ ID NO: 69, 36, or 981;
(ii) an amino acid sequence at least 95% or at least 98% identical to amino acids 138-742 of SEQ ID NO: 69, 36, or 981; and/or
(iii) an amino acid sequence at least 95% or at least 98% identical to the amino acid sequence of SEQ ID NO: 69, 36, or 981.

15. The AAV particle of any one of claims 1-14, wherein the AAV capsid variant comprises:
(i) the amino acid sequence of amino acids 203-742 of SEQ ID NO: 69, 36, or 981;
(ii) the amino acid sequence of amino acids 138-742 of SEQ ID NO: 69, 36, or 981; and/or
(iii) the amino acid sequence of SEQ ID NO: 69, 36, or 981.

16. An AAV particle comprising an AAV capsid variant and a nucleic acid encoding a polynucleotide that targets human SOD1, wherein the AAV capsid variant comprises:
(i) the amino acid sequence of HDSPHK (SEQ ID NO: 2) in hypervariable loop IV; and
(ii) an amino acid sequence at least 95% identical to the amino acid sequence of positions 203-736 of SEQ ID NO: 138.

17. The AAV particle of claim 16, wherein the amino acid sequence of HDSPHK (SEQ ID NO: 2) is present immediately subsequent to amino acid 453, numbered according to SEQ ID NO: 982.

18. The AAV particle of any one of claim 1-9, 16, or 17, wherein the AAV capsid variant comprises:
(i) an amino acid sequence at least 95% or at least 98% identical to amino acids 203-742 of SEQ ID NO: 982, 37, or 6;
(ii) an amino acid sequence at least 95% or at least 98% identical to amino acids 138-742 of SEQ ID NO: 982, 37, or 6; and/or
(iii) an amino acid sequence at least 95% or at least 98% identical to the amino acid sequence of SEQ ID NO: 982, 37, or 6.

19. The AAV particle of any one of claim 1-9 or 16-18, wherein the AAV capsid variant comprises:
(i) the amino acid sequence of amino acids 203-742 of SEQ ID NO: 982, 37, or 6;
(ii) the amino acid sequence of amino acids 138-742 of SEQ ID NO: 982, 37, or 6; and/or
(iii) the amino acid sequence of SEQ ID NO: 982, 37, or 6.

20. The AAV particle of any one of claims 1-19, wherein hypervariable loop IV comprises amino acids 449-460, numbered according to SEQ ID NO: 138.

21. The AAV particle of any one of claims 1-20, wherein the AAV capsid variant:
(i) has an increased tropism for a CNS cell or tissue, e.g., a brain cell, brain tissue, spinal cord cell, or spinal cord tissue, relative to the tropism of a reference sequence comprising the amino acid sequence of SEQ ID NO: 138;
(ii) transduces a brain region (e.g., a putamen, caudate, entorhinal cortex, hippocampus, thalamus, substantia nigra, motor cortex, frontal cortex, temporal cortex, cerebral cortex, dentate nucleus, and/or Lateral Geniculate Nucleus (LGN)), optionally wherein the level of transduction is at least 5, 10, 15, 20, 25, 30, 35, 40, 45, 50, 55, 60, or 65-fold greater as compared to a reference sequence of SEQ ID NO: 138, e.g., when measured by an assay, e.g., an immunohistochemistry assay or a qPCR assay, e.g., as described in Example 3 or 7;
(iii) is enriched at least about 3, 4, 5, 6, 7, 8, 9, 10, 20, 30, 40, 50, 60, 70, 80, 90, 100, 110, 120, 130, 140, 150, 160, 170, 180, 190, 200, or 210-fold, in the brain compared to a reference sequence of SEQ ID NO: 138, e.g., when measured by an assay as described in Example 1 or 4;
(iv) has increased tropism for a CNS cell or tissue, e.g., a brain cell, brain tissue, spinal cord cell, or spinal cord tissue, of at least two to three species, e.g., a non-human primate and rodent (e.g., Macaca fascicularis, Chlorocebus sabaeus, Callithrix jacchus, and/or mouse (e.g., BALB/c mice, C57B1/6 mice, and/or CD-1 outbred mice), relative to the tropism of a reference sequence comprising the amino acid sequence of SEQ ID NO: 138;
(v) is enriched at least about 10, 15, 20, 25, 30, 35, 40, 45, 50, 55, 60, 65, 70, 75, 80, 85, 90, 95, or 100-fold, in the brain of at least two to three species, e.g., a non-human primate and rodent (e.g., Macaca fascicularis, Chlorocebus sabaeus, Callithrix jacchus, and/or mouse (e.g., BALB/c mice, C57B1/6 mice, and/or CD-1 outbred mice), compared to a reference sequence of SEQ ID NO:
138, e.g., when measured by an assay as described in Example 2, 5, or 8;
(vi) delivers an increased level of a payload to a brain region, optionally wherein the level of the payload is increased by at least 5, 10, 15, 17, 18, 19, 20, 25, 30, 35, 40, 45, 50, 55, 60, 65, or 70-fold, as compared to a reference sequence of SEQ ID NO: 138, e.g., when measured by an assay, e.g., a qRT-PCR or a qPCR assay (e.g., as described in Example 2 or 8), optionally wherein the brain region is a midbrain region (e.g., the hippocampus or thalamus), frontal cortex, temporal cortex, motor cortex, cerebral cortex, caudate, putamen, dentate nucleus, substantia nigra, or the brainstem;
(vii) delivers an increased level of viral genomes to a brain region, optionally wherein the level of viral genomes is increased by at least 5, 10, 15, 17, 18, 19, 20, 25, 30, 35, 40, 45, or 50-fold, as compared to a reference sequence of SEQ ID NO: 138, e.g., when measured by an assay, e.g., a qRT-PCR or a qPCR assay (e.g., as described in Example 2 or 8), optionally wherein the brain region is a putamen, caudate, hippocampus, thalamus, substantia nigra, motor cortex, frontal cortex, temporal cortex, cerebral cortex, dentate nucleus, and/or Lateral Geniculate Nucleus (LGN));
(viii) is enriched at least about 5, 10, 15, 20, 25, 30, or 35-fold, in the spinal cord compared to a reference sequence of SEQ ID NO: 138, e.g., when measured by an assay as described in Example 1 or 8, optionally wherein the region of the spinal cord is a thoracic spinal cord region, cervical spinal cord region, C5 ventral horn region, lumbar spinal cord region, or L5 ventral horn region;
(ix) shows preferential transduction in a brain region relative to the transduction in the dorsal root ganglia (DRG) and/or the liver;
(x) is capable of transducing neuronal cells and/or non-neuronal cells (e.g., astrocytes);
(xi) is capable of transducing at least 20%, 25%, 30%, 40%, 50%, 55%, 60%, 65%, 70%, 75%, 80%, 85, 90%, or 95% of cells in a brain region (e.g., a putamen, caudate, entorhinal cortex, hippocampus, thalamus, substantia nigra, motor cortex, frontal cortex, temporal cortex, cerebral cortex, cerebellar cortex, cerebellum, dentate nucleus, and/or Lateral Geniculate Nucleus (LGN)), e.g., when measured by an assay as described in Example 7;
(xii) is capable of transducing at least 45%, 50%, 55%, 60%, 65%, 70%, 75%, 80%, 85%, 90%, 95%, 97%, or 99% of astrocytes (e.g., Sox9+astrocytes) in a brain region (e.g., a putamen, caudate, entorhinal cortex, hippocampus, thalamus, substantia nigra, motor cortex, frontal cortex, or temporal cortex), e.g., when measured by an assay as described in Example 7;
(xiii) is capable of transducing at least 25%, 30%, 35%, 40%, 45% 50%, 55%, 60%, 65%, or 70% of neurons (e.g., NeuN+neurons) in a brain region (e.g., a putamen, caudate, entorhinal cortex, hippocampus, thalamus, substantia nigra, motor cortex, frontal cortex, or temporal cortex), e.g., when measured by an assay as described in Example 7; and/or
(xiv) is capable of transducing at least 70%, 75%, 80%, 85%, 90%, 91%, 92%, 93%, 94%, or 95%, 96%, or 97% of astrocytes (e.g., Sox9+astrocytes) in the spinal cord (e.g., the cervical spinal cord, the thoracic spinal cord, or the lumbar spinal cord), e.g., when measured by an assay as described in Example 7.

22. The AAV particle of any one of claims 1-21, wherein the encoded polynucleotide comprises a siRNA comprising a sense strand sequence and an antisense strand sequence, wherein the antisense strand sequence is fully or partially complementary to human SOD1.

23. The AAV particle of claim 22, wherein:
(i) the encoded sense strand nucleotide sequence comprises at least 15, 16, 17, 18, 19, 20, or 21 contiguous nucleotides differing by no more than 3, 2, 1, or 0 nucleotides from a sense strand nucleotide sequence comprising any one of the sense strand sequences of Table 10 or 14; and/or
(ii) the encoded antisense strand sequence comprises at least 15, 16, 17, 18, 19, 20, 21, or contiguous nucleotides differing by no more than 3, 2, 1, or 0 nucleotides from any one of the antisense strand sequences of Table 10 or 14;
wherein the encoded sense strand sequence and the encoded antisense strand sequence comprise a region of complementarity of at least 15 nucleotides.

24. The AAV particle of claim 22 or 23, wherein:
(i) the encoded sense strand sequence comprises at least 18, 19, 20, or 21 contiguous nucleotides differing by no more than 3, 2, 1 or 0 nucleotides from SEQ ID NO: 2525 or 2381;
(ii) the encoded antisense strand sequence comprises at least 18, 19, 20, or 21 contiguous nucleotides differing by no more than 3, 2, 1 or 0 nucleotides from SEQ ID NO: 2507 or 2363;
(iii) the encoded sense strand sequence comprises at least 18, 19, 20, or 21 contiguous nucleotides differing by no more than 3, 2, 1 or 0 nucleotides from SEQ ID NO: 2525; and the encoded antisense strand sequence comprises at least 18, 19, 20, or 21 contiguous nucleotides differing by no more than 3, 2, 1 or 0 nucleotides SEQ ID NO: 2507; or
(iv) the encoded sense strand sequence comprises at least 18, 19, 20, or 21 contiguous nucleotides differing by no more than 3, 2, 1 or 0 nucleotides SEQ ID NO: 2381, and the encoded antisense strand sequence comprises at least 18, 19, 20, or 21 contiguous nucleotides differing by no more than 3, 2, 1 or 0 nucleotides SEQ ID NO: 2363.

25. The AAV particle of any one of claims 22-24, wherein:
(i) the encoded sense strand sequence comprises SEQ ID NO: 2525, and the encoded antisense strand sequence comprises SEQ ID NO: 2507; or
(ii) the encoded sense strand sequence comprises SEQ ID NO: 2381, and the encoded antisense strand sequence comprises SEQ ID NO: 2363.

26. The AAV particle of any one of claims 22-25, wherein:
(i) the nucleotide sequence encoding the antisense strand sequence comprises the nucleotide sequence of SEQ ID NO: 1351 and the nucleotide sequence encoding the sense strand sequence comprises the nucleotide sequence of SEQ ID NO: 66; or
(ii) the nucleotide sequence encoding the antisense strand sequence comprises the nucleotide sequence of SEQ ID NO: 1368 and the nucleotide sequence encoding the sense strand sequence comprises the nucleotide sequence of SEQ ID NO: 1347.

27. An AAV particle comprising:
(i) an AAV capsid variant comprising the amino acid sequence of SEQ ID NO: 69; and
(ii) a nucleic acid encoding a polynucleotide targeting human SOD1, wherein the encoded polynucleotide comprises a siRNA comprising a sense strand sequence and an antisense strand sequence, wherein the encoded sense strand sequence comprises SEQ ID NO: 2525, and the encoded antisense strand sequence comprises SEQ ID NO: 2507.

28. An AAV particle comprising:
(i) an AAV capsid variant comprising the amino acid sequence of SEQ ID NO: 36; and
(ii) a nucleic acid encoding a polynucleotide targeting human SOD1, wherein the encoded polynucleotide comprises a siRNA comprising a sense strand sequence and an antisense strand sequence, wherein the encoded sense strand sequence comprises SEQ ID NO: 2525, and the encoded antisense strand sequence comprises SEQ ID NO: 2507.

29. An AAV particle comprising:
(i) an AAV capsid variant comprising the amino acid sequence of SEQ ID NO: 982; and
(ii) a nucleic acid encoding a polynucleotide targeting human SOD1, wherein the encoded polynucleotide comprises a siRNA comprising a sense strand sequence and an antisense strand sequence, wherein the encoded sense strand sequence comprises SEQ ID NO: 2525, and the encoded antisense strand sequence comprises SEQ ID NO: 2507.

30. The AAV particle of any one of claims 22-29, wherein:
(i) at least one of the sense strand sequence and the antisense strand sequence comprise a 3′ overhang of at least 1, or 2 nucleotides; and/or
(ii) the sense strand sequence, the antisense strand sequence or both, each independently comprise 15 to 30 nucleotides in length, 15 to 25 nucleotides in length, 17 to 22 nucleotides, or 17-20 nucleotides in length.

31. The AAV particle of any one of claims 22-30, wherein the encoded polynucleotide further comprises a modulatory polynucleotide comprising the siRNA, wherein the encoded modulatory polynucleotide comprises:
(i) a 5′ flanking region;
(ii) a loop region; and/or
(iii) a 3′ flanking region.

32. The AAV particle of claim 31, wherein:
(A) (i) the encoded 5′ flanking region comprises the nucleotide sequence of any one of SEQ ID NOs: 5000-5003; a nucleotide sequence at least 90%, 92%, 95%, 97%, 98%, or 99% identical to the nucleotide sequence of any one of SEQ ID NOs: 5000-5003; or a nucleotide sequence having at least one, two, three, four, five, six, or seven modifications, but no more than ten modifications relative to the nucleotide sequence any one of SEQ ID NOs: 5000-5003;
(ii) the encoded loop region comprises the nucleotide sequence of any one of SEQ ID NOs: 5004-5007; a nucleotide sequence at least 90%, 92%, 95%, 97%, 98%, or 99% identical to the nucleotide sequence any one of SEQ ID NOs: 5004-5007; or a nucleotide sequence having at least one, two, three, or four, modifications, but no more than six modifications relative to the nucleotide sequence of any one of SEQ ID NOs: 5004-5007; and/or
(iii) the encoded 3′ flanking region comprises the nucleotide sequence of any one of SEQ ID NOs: 5008-5012; a nucleotide sequence at least 90%, 92%, 95%, 97%, 98%, or 99% identical to the nucleotide sequence of any one of SEQ ID NOs: 5008-5012; or a nucleotide sequence having at least one, two, three, four, five, six, or seven modifications, but no more than ten modifications relative to the nucleotide sequence of any one of SEQ ID NOs: 5008-5012; and/or
(B) (i) the nucleotide sequence encoding the 5′ flanking region comprises the nucleotide sequence of any one of SEQ ID NOs: 2547-2549 or 5014; a nucleotide sequence at least 90%, 92%, 95%, 97%, 98%, or 99% identical to the nucleotide sequence of any one of SEQ ID NOs: 2547-2549 or 5014; or
a nucleotide sequence having at least one, two, three, four, five, six, or seven modifications, but no more than ten modifications relative to the nucleotide sequence any one of SEQ ID NOs: 2547-2549 or 5014;
(ii) the nucleotide sequence encoding the loop region comprises the nucleotide sequence of any one of SEQ ID NOs: 2550-2553; a nucleotide sequence at least 90%, 92%, 95%, 97%, 98%, or 99% identical to the nucleotide sequence any one of SEQ ID NOs: 2550-2553; or a nucleotide sequence having at least one, two, three, or four, modifications, but no more than six modifications relative to the nucleotide sequence of any one of SEQ ID NOs: 2550-2553; and/or
(iii) the nucleotide sequence encoding the 3′ flanking region comprises the nucleotide sequence of any one of SEQ ID NOs: 2554-2558; a nucleotide sequence at least 90%, 92%, 95%, 97%, 98%, or 99% identical to the nucleotide sequence of any one of SEQ ID NOs: 2554-2558; or a nucleotide sequence having at least one, two, three, four, five, six, or seven modifications, but no more than ten modifications relative to the nucleotide sequence of any one of SEQ ID NOs: 2554-2558.

33. The AAV particle of claim 31 or 32, wherein:
(A) (i) the encoded 5′ flanking region comprises the nucleotide sequence of SEQ ID NO:
5000, a nucleotide sequence at least 90%, 92%, 95%, 96%, 97%, 98%, or 99% identical to SEQ ID NO: 5000, a nucleotide sequence comprising one, two, three, four, five, six, or seven, but no more than ten different nucleotides relative to the nucleotide sequence of SEQ ID NO: 5000; or a nucleotide sequence comprising one, two, three, four, five, six, or seven, but no more than ten different nucleotides relative to the nucleotide sequence of SEQ ID NO: 5000;
(ii) the encoded loop region comprises the nucleotide sequence of SEQ ID NO: 5004, a nucleotide sequence at least 90%, 92%, 95%, 96%, 97%, 98%, or 99% identical to SEQ ID NO:
5004, a nucleotide sequence comprising one, two, three, or four, but no more than five different nucleotides relative to the nucleotide sequence of SEQ ID NO: 5004; or a nucleotide sequence comprising one, two, three, or four, but no more than five different nucleotides relative to the nucleotide sequence of SEQ ID NO: 5004; and
(iii) the encoded 3′ flanking region comprises the nucleotide of SEQ ID NO: 5008, a nucleotide sequence at least 90%, 92%, 95%, 96%, 97%, 98%, or 99% identical to SEQ ID NO: 5008, a nucleotide sequence comprising one, two, three, four, five, six, or seven, but no more than ten different nucleotides relative to the nucleotide sequence of SEQ ID NO: 5008, a nucleotide sequence comprising one, two, three, four, five, six, or seven, but no more than ten modifications relative to the nucleotide sequence of SEQ ID NO: 5008; and/or
(B) (i) the nucleotide sequence encoding the 5′ flanking region comprises the nucleotide sequence of SEQ ID NO: 2547, a nucleotide sequence at least 90%, 92%, 95%, 96%, 97%, 98%, or 99% identical to SEQ ID NO: 2547, a nucleotide sequence comprising one, two, three, four, five, six, or seven, but no more than ten different nucleotides relative to the nucleotide sequence of SEQ ID NO: 2547; or a nucleotide sequence comprising one, two, three, four, five, six, or seven, but no more than ten different nucleotides relative to the nucleotide sequence of SEQ ID NO: 2547;
(ii) the nucleotide sequence encoding the loop region comprises the nucleotide sequence of SEQ ID NO: 2550, a nucleotide sequence at least 90%, 92%, 95%, 96%, 97%, 98%, or 99% identical to SEQ ID NO: 2550, a nucleotide sequence comprising one, two, three, or four, but no more than five different nucleotides relative to the nucleotide sequence of SEQ ID NO: 2550; or a nucleotide sequence comprising one, two, three, or four, but no more than five different nucleotides relative to the nucleotide sequence of SEQ ID NO: 2550; and
(iii) the nucleotide sequence encoding the 3′ flanking region comprises the nucleotide sequence of SEQ ID NO: 2554, a nucleotide sequence at least 90%, 92%, 95%, 96%, 97%, 98%, or 99% identical to SEQ ID NO: 2554, a nucleotide sequence comprising one, two, three, four, five, six, or seven, but no more than ten different nucleotides relative to the nucleotide sequence of SEQ ID NO: 2554, a nucleotide sequence comprising one, two, three, four, five, six, or seven, but no more than ten modifications relative to the nucleotide sequence of SEQ ID NO: 2554.

34. The AAV particle of claim 31 or 32, wherein:
(A) (i) the encoded 5′ flanking region comprises the nucleotide sequence of SEQ ID NO:
5001, a nucleotide sequence at least 90%, 92%, 95%, 96%, 97%, 98%, or 99% identical to SEQ ID NO: 5001, a nucleotide sequence comprising one, two, three, four, five, six, or seven, but no more than ten different nucleotides relative to the nucleotide sequence of SEQ ID NO: 5001; or a nucleotide sequence comprising one, two, three, four, five, six, or seven, but no more than ten different nucleotides relative to the nucleotide sequence of SEQ ID NO: 5001;
(ii) the encoded loop region comprises the nucleotide sequence of SEQ ID NO: 5005, a nucleotide sequence at least 90%, 92%, 95%, 96%, 97%, 98%, or 99% identical to SEQ ID NO:
5005, a nucleotide sequence comprising one, two, three, or four, but no more than five different nucleotides relative to the nucleotide sequence of SEQ ID NO: 5005; or a nucleotide sequence comprising one, two, three, or four, but no more than five different nucleotides relative to the nucleotide sequence of SEQ ID NO: 5005; and
(iii) the encoded 3′ flanking region comprises the nucleotide of SEQ ID NO: 5009, a nucleotide sequence at least 90%, 92%, 95%, 96%, 97%, 98%, or 99% identical to SEQ ID NO:
5009, a nucleotide sequence comprising one, two, three, four, five, six, or seven, but no more than ten different nucleotides relative to the nucleotide sequence of SEQ ID NO: 5009, a nucleotide sequence comprising one, two, three, four, five, six, or seven, but no more than ten modifications relative to the nucleotide sequence of SEQ ID NO: 5009; and/or
(B) (i) the nucleotide sequence encoding the 5′ flanking region comprises the nucleotide sequence of SEQ ID NO: 2548, a nucleotide sequence at least 90%, 92%, 95%, 96%, 97%, 98%, or 99% identical to SEQ ID NO: 2548, a nucleotide sequence comprising one, two, three, four, five, six, or seven, but no more than ten different nucleotides relative to the nucleotide sequence of SEQ ID NO: 2548; or a nucleotide sequence comprising one, two, three, four, five, six, or seven, but no more than ten different nucleotides relative to the nucleotide sequence of SEQ ID NO: 2548;
(ii) the nucleotide sequence encoding the loop region comprises the nucleotide sequence of SEQ ID NO: 2551, a nucleotide sequence at least 90%, 92%, 95%, 96%, 97%, 98%, or 99% identical to SEQ ID NO: 2551, a nucleotide sequence comprising one, two, three, or four, but no more than five different nucleotides relative to the nucleotide sequence of SEQ ID NO: 2551; or a nucleotide sequence comprising one, two, three, or four, but no more than five different nucleotides relative to the nucleotide sequence of SEQ ID NO: 2551; and
(iii) the nucleotide sequence encoding the 3′ flanking region comprises the nucleotide sequence of SEQ ID NO: 2555, a nucleotide sequence at least 90%, 92%, 95%, 96%, 97%, 98%, or 99% identical to SEQ ID NO: 2555, a nucleotide sequence comprising one, two, three, four, five, six, or seven, but no more than ten different nucleotides relative to the nucleotide sequence of SEQ ID NO: 2555, a nucleotide sequence comprising one, two, three, four, five, six, or seven, but no more than ten modifications relative to the nucleotide sequence of SEQ ID NO: 2555.

35. The AAV particle of any one of claims 31-34, wherein:
(i) the nucleotide sequence encoding the modulatory polynucleotide comprises the nucleotide sequence of any one of SEQ ID NOs: 2562, 2579, 5022, 2559, 2560, 2561, 2563, 2564, 2565, 2566, 2567, 2568, 2569, 2570, 2571, 2572, 2573, 2574, 2575, 2576, 2577, 2578 or a nucleotide sequence at least 90%, 92%, 95%, 97%, 98%, or 99% identical thereto;
(ii) the nucleotide sequence encoding the modulatory polynucleotide comprises the nucleotide sequence of SEQ ID NO: 2562, or a nucleotide sequence at least 90%, 92%, 95%, 97%, 98%, or 99% identical thereto;
(iii) the nucleotide sequence encoding the modulatory polynucleotide comprises the nucleotide sequence of SEQ ID NO: 2579, or a nucleotide sequence at least 90%, 92%, 95%, 97%, 98%, or 99% identical thereto;
(iv) the encoded modulatory polynucleotide comprises the nucleotide sequence of SEQ ID NO: 5013, or a nucleotide sequence at least 90%, 92%, 95%, 97%, 98%, or 99% identical thereto; or
(v) the encoded modulatory polynucleotide comprises the nucleotide sequence of SEQ ID NO: 4821, or a nucleotide sequence at least 90%, 92%, 95%, 97%, 98%, or 99% identical thereto.

36. An AAV particle comprising:
(i) an AAV capsid variant comprising the amino acid sequence of SEQ ID NO: 69; and
(ii) a nucleic acid encoding a polynucleotide targeting human SOD1, wherein the encoded polynucleotide comprises the nucleotide sequence of SEQ ID NO: 5013.

37. An AAV particle comprising:
(i) an AAV capsid variant comprising the amino acid sequence of SEQ ID NO: 36; and
(ii) a nucleic acid encoding a polynucleotide targeting human SOD1, wherein the encoded polynucleotide comprises the nucleotide sequence of SEQ ID NO: 5013.

38. An AAV particle comprising:
(i) an AAV capsid variant comprising the amino acid sequence of SEQ ID NO: 982; and
(ii) a nucleic acid encoding a polynucleotide targeting human SOD1, wherein the encoded polynucleotide comprises the nucleotide sequence of SEQ ID NO: 5013.

39. The AAV particle of any one of claims 1-38, which comprises a viral genome comprising a promoter operably linked to the nucleic acid encoding the polynucleotide.

40. The AAV particle of claim 39, the promoter comprises:
(i) a chicken β-actin (CBA) promoter and/or its derivative CAG, an EF-1a promoter, a CMV immediate-early enhancer and/or promoter, a β glucuronidase (GUSB) promoter, a ubiquitin C (UBC) promoter, a neuron-specific enolase (NSE), a platelet-derived growth factor (PDGF) promoter, a platelet-derived growth factor B-chain (PDGF-B) promoter, an intercellular adhesion molecule 2 (ICAM-2) promoter, a synapsin (Syn) promoter, a methyl-CpG binding protein 2 (MeCP2) promoter, a Ca²⁺/calmodulin-dependent protein kinase II (CaMKII) promoter, a metabotropic glutamate receptor 2 (mGluR2) promoter, a neurofilament light (NFL) or heavy (NFH) promoter, a β-globin minigene nβ2 promoter, a preproenkephalin (PPE) promoter, an enkephalin (Enk) and excitatory amino acid transporter 2 (EAAT2), a glial fibrillary acidic protein (GFAP) promoter, a myelin basic protein (MBP) promoter, a cardiovascular promoter (e.g., αMHC, cTnT, and CMV-MLC2k), a liver promoter (e.g., hAAT, TBG), a skeletal muscle promoter (e.g., desmin, MCK, C512) or a fragment, e.g., a truncation, or a functional variant thereof; or
(iv) an H1 promoter comprising the nucleotide sequence of SEQ ID NO: 83, or a nucleotide sequence at least 90%, 95%, 96%, 97%, 98%, or 99% identical to SEQ ID NO: 83.

41. The AAV particle of claim 39 or 40, wherein the viral genome further comprises one, two, three, four, five, six, seven, or all of:
(i) an inverted terminal repeat (ITR), optionally wherein the viral genome comprises an ITR positioned 5′ relative to the encoded polynucleotide, and/or an ITR positioned 3′ relative to the encoded polynucleotide;
(ii) an enhancer;
(iii) a miR binding site;
(iv) a polyadenylation (polyA) signal region;
(v) an intron region;
(vi) a filler sequence;
(vii) an exon region; and
(viii) a Kozak sequence.

42. The AAV particle of any one of claims 39-41, which comprises:
(i) an ITR comprising the nucleotide sequence comprises the nucleotide sequence of SEQ ID NO: 126 or 130, or a nucleotide sequence at least 90%, 95%, 96%, 97%, 98%, or 99% identical thereto;
(ii) a polyA signal region comprising the nucleotide sequence of SEQ ID NO: 129, or a nucleotide sequence at least 90%, 95%, 96%, 97%, 98%, or 99% identical thereto; and/or
(iii) a filler sequence comprising the nucleotide sequence of SEQ ID NO: 82, or a nucleotide sequence at least 90%, 95%, 96%, 97%, 98%, or 99% identical thereto.

43. The AAV particle of any one of claims 39-42, wherein the viral genome comprises, from 5′ to 3′:
(a) an ITR;
(b) a filler sequence;
(b) the promoter;
(d) the nucleic acid encoding the polynucleotide targeting human SOD1;
(c) a polyA signal region; and
(f) an ITR.

44. The AAV particle of any one of claims 39-43, wherein the viral genome comprises, from 5′ to 3′:
(a) an ITR comprising the nucleotide sequence of SEQ ID NO: 126, or a nucleotide sequence at least 90%, 95%, 96%, 97%, 98%, or 99% identical thereto;
(b) a filler sequence comprising the nucleotide sequence of SEQ ID NO: 82, or a nucleotide sequence at least 90%, 95%, 96%, 97%, 98%, or 99% identical thereto;
(b) the promoter comprising the nucleotide sequence of SEQ ID NO: 83, or a nucleotide sequence at least 90%, 95%, 96%, 97%, 98%, or 99% identical thereto;
(d) the nucleic acid encoding the polynucleotide targeting human SOD1 comprising the nucleotide sequence of SEQ ID NO: 2562, or a nucleotide sequence at least 90%, 95%, 96%, 97%, 98%, or 99% identical thereto;
(e) a polyA signal region comprising the nucleotide sequence of SEQ ID NO: 129, or a nucleotide sequence at least 90%, 95%, 96%, 97%, 98%, or 99% identical thereto; and
(f) an ITR comprising the nucleotide sequence of SEQ ID NO: 130, or a nucleotide sequence at least 90%, 95%, 96%, 97%, 98%, or 99% identical thereto.

45. The AAV particle of any one of claims 39-44, wherein the viral genome comprises:
(i) the nucleotide sequence of any one of SEQ ID NOs: 109, 125, 4820, or 4028-4041; a nucleotide sequence comprising at least 1, 2, 3, 4, or 5 but no more than 30, 20, or 10 different nucleotides, relative to the nucleotide sequence of any one of SEQ ID NOs: 109, 125, or 4028-4041; or a nucleotide sequence at least 90%, 95%, 99% or 100% identical to any one of SEQ ID NOs: 109, 125, 4820, or 4028-4041;
(ii) the nucleotide sequence of SEQ ID NO: 109; a nucleotide sequence comprising at least 1, 2, 3, 4, or 5, but no more than 30, 20, or 10 different nucleotides, relative to SEQ ID NO: 109; or a nucleotide sequence at least 90%, 95%, 99% or 100% identical to SEQ ID NO: 109;
(iii) the nucleotide sequence of SEQ ID NO: 125; a nucleotide sequence comprising at least 1, 2, 3, 4, or 5 but no more than 30, 20, or 10 different nucleotides, relative to SEQ ID NO: 125; or a nucleotide sequence at least 90%, 95%, 99% or 100% identical to SEQ ID NO: 125; or
(iv) the nucleotide sequence of SEQ ID NO: 4820; a nucleotide sequence comprising at least 1, 2, 3, 4, or 5 but no more than 30, 20, or 10 different nucleotides, relative to SEQ ID NO: 4820; or a nucleotide sequence at least 90%, 95%, 99% or 100% identical to SEQ ID NO: 4820.

46. An AAV particle comprising:
(i) an AAV capsid variant comprising the amino acid sequence of SEQ ID NO: 69; and
(ii) a viral genome comprising the nucleotide sequence of SEQ ID NO: 109.

47. An AAV particle comprising:
(i) an AAV capsid variant comprising the amino acid sequence of SEQ ID NO: 36; and
(ii) a viral genome comprising the nucleotide sequence of SEQ ID NO: 109.

48. An AAV particle comprising:
(i) an AAV capsid variant comprising the amino acid sequence of SEQ ID NO: 982; and
(ii) a viral genome comprising the nucleotide sequence of SEQ ID NO: 109.

49. The AAV particle of any one of claims 39-48, wherein the viral genome is self-complementary or single-stranded.

50. A cell, e.g., a host cell, comprising the AAV particle of any one of claims 1-49, optionally wherein the cell is:
(i) a cell of the brain or the spinal cord (e.g., a cell of a putamen, caudate, hippocampus, thalamus, substantia nigra, motor cortex, frontal cortex, temporal cortex, cerebral cortex, dentate nucleus, Lateral Geniculate Nucleus (LGN), cervical spinal cord, lumbar spinal cord, and/or thoracic spinal cord);
(ii) a neuron (e.g., a motor neuron); and/or
(iii) an astrocyte.

51. A method of making the AAV particle of any one of claims 1-49, comprising:
(i) providing a host cell comprising a viral genome; and
(ii) incubating the host cell under conditions suitable to enclose the viral genome in the AAV capsid variant;
thereby making the AAV particle.

52. A pharmaceutical composition comprising the AAV particle of any one of claims 1-49, and a pharmaceutically acceptable excipient.

53. A method of delivering a polynucleotide targeting human SOD1 to a subject, comprising administering an effective amount of the pharmaceutical composition of claim 52, or the AAV particle of any one of claims 1-49 to the subject, thereby delivering polynucleotide targeting human SOD1 to the subject.

54. A method of treating a subject having or diagnosed with having a neurological disorder or a neurodegenerative disorder related to expression of a SOD1 gene, mRNA, or protein (e.g., aberrant expression of SOD1 gene, mRNA, or protein), comprising administering to the subject an effective amount of the pharmaceutical composition of claim 52, or the AAV of any one claims 1-49, thereby treating the subject.

55. The method of claim 54, wherein the disease or disorder is amyotrophic lateral sclerosis (ALS).

56. The method of claim 53, wherein the subject has ALS.

57. The method of claim 55 or 56, wherein the ALS is:
(i) familial ALS;
(ii) sporadic ALS;
(iii) early stage ALS;
(iv) middle stage ALS; and/or
(v) late stage ALS.

58. The method of any one of claims 55-57 wherein treatment comprises amelioration of a symptom of ALS in the subject, optionally, wherein the symptom comprises motor neuron degeneration, muscle weakness, stiffness of muscles, muscle atrophy, muscle stiffness, fasciculation development, frontotemporal dementia, slurred speech, difficulty breathing, or a combination thereof.

59. The method of any one of claims 53-58, wherein the AAV particle or the pharmaceutical composition is administered to the subject intravenously, intracerebrally, via intrathalamic (ITH) administration, intramuscularly, intrathecally, intracerebroventricularly, via intraparenchymal administration, via focused ultrasound (FUS), e.g., coupled with the intravenous administration of microbubbles (FUS-MB), or MRI-guided FUS coupled with intravenous administration, via intra-cisterna magna injection (ICM), or via dual ITH and ICM administration.

60. The method of any one of claims 53-59, wherein administration of the AAV particle or pharmaceutical composition results in at least a 30-60% reduction of SOD1 mRNA expression, e.g., in the spinal cord of the subject, relative to a subject that has not received the AAV particle or pharmaceutical composition.

61. Use of the pharmaceutical composition of claim 52, or the AAV particle of claims 1-49, in the manufacture of a medicament for treating a neurological disorder, a neurodegenerative disorder, a disease associated with expression of a SOD1 gene, mRNA, or protein (e.g., aberrant expression of SOD1 gene, mRNA, or protein) (e.g., ALS).

62. The pharmaceutical composition of claim 52, or the AAV particle of claims 1-49, for use in a method of treating a neurological disorder, a neurodegenerative disorder, a disease associated with expression of a SOD1 gene, mRNA, or protein (e.g., aberrant expression of SOD1 gene, mRNA, or protein) (e.g., ALS).
